(19) United States
(12) Patent Application Publication
(10) Pub. No.: US 2025/0248975 A1
(43) Pub. Date: Aug. 7, 2025

(54) MACROCYCLIC COMPOUNDS, COMPOSITIONS, AND METHODS OF USING THEREOF

(71) Applicants: Sionna Therapeutics Inc., Waltham, MA (US); Genzyme Corporation, Cambridge, MA (US)

(72) Inventors: Junkai Liao, Andover, MA (US); John E. Macor, Washington Crossing, PA (US); George Topalov, Pittsburgh, PA (US); Mark Munson, Acton, MA (US); Sukanthini Thurairatnam, Dover, MA (US); Bradford Hirth, Littleton, MA (US); Zhongli Gao, Waltham, MA (US); Gregory Donald Hurlbut, Boxborough, MA (US); Andrew Good, Bideford (GB); Roy Vaz, Southborough, MA (US); Jinyu Liu, Union City, CA (US); Yi Li, Weston, MA (US); Anatoly Ruvinsky, Lexington, MA (US); Michael Kothe, Medway, MA (US); David Borcherding, Bangor, PA (US); Patrick Bernardelli, Paris (FR); Arielle Genevois Borella, Paris (FR); Franck Caussanel, Paris (FR); Ingrid Devillers, Paris (FR); Eric Nicolai, Paris (FR); Franck Slowinski, Paris (FR); Fabienne Thompson, Paris (FR); Lothar Schwink, Frankfurt am Main (DE); Heiner Glombik, Frankfurt am Main (DE); Stefan Guessregen, Frankfurt am Main (DE); Michael Podeschwa, Frankfurt am Main (DE); Nils Rackelmann, Frankfurt am Main (DE); Sven Ruf, Frankfurt am Main (DE)

(21) Appl. No.: 19/071,578

(22) Filed: Mar. 5, 2025

Related U.S. Application Data
(63) Continuation of application No. PCT/US2023/073558, filed on Sep. 6, 2023.
(60) Provisional application No. 63/404,439, filed on Sep. 7, 2022.

(51) Int. Cl. A61K 31/4192; A61K 31/439; C07D 498/22; C07D 513/22; C07D 515/22

(52) U.S. Cl. CPC A61K 31/4192; A61K 31/439; C07D 498/22; C07D 513/22; C07D 515/22

(57) ABSTRACT

The present disclosure includes, among other things, CFTR modulators, pharmaceutical compositions, and methods of making and using the same.

This application is a continuation of International Application No. PCT/US2023/073558, filed Sep. 6, 2023, which claims the benefit of and priority to U.S. Provisional Patent Application No. 63/404,439, filed Sep. 7, 2022, each of which are hereby incorporated by reference in their entireties for all purposes.

### BACKGROUND

Cystic fibrosis (CF), an autosomal recessive disorder, is caused by functional deficiency of the cAMP-activated plasma membrane chloride channel, cystic fibrosis transmembrane conductance regulator (CFTR), which results in pulmonary and other complications. The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). CFTR, a member of the ATP binding cassette (ABC) superfamily is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate, and thiocyanate into and out of the cell. CFTR may have a regulatory role over other electrolyte channels, including the epithelial sodium channel ENaC.

In cystic fibrosis patients, the absence or dysfunction of CFTR leads to exocrine gland dysfunction and a multisystem disease, characterized by pancreatic insufficiency and malabsorption, as well as abnormal mucociliary clearance in the lung, mucostasis, chronic lung infection and inflammation, decreased lung function and ultimately respiratory failure.

While more than 1,900 mutations have been identified in the CFTR gene, a detailed understanding of how each CFTR mutation may impact channel function is known for only a few. (Derichs, European Respiratory Review, 22:127, 58-65 (2013)). The most frequent CFTR mutation is the in-frame deletion of phenylalanine at residue 508 (ΔF508) in the first nucleotide binding domain (NBD1). Over 70% of cystic fibrosis patients have a deletion at residue 508 in at least one CFTR allele. The loss of this key phenylalanine renders NBD1 conformationally unstable at physiological temperature and compromises the integrity of the interdomain interface between NDB1 and CFTR's second transmembrane domain (ICL4). The ΔF508 mutation causes production of misfolded CFTR protein which, rather than traffic to the plasma membrane, is instead retained in the endoplasmic reticulum and targeted for degradation by the ubiquitin-proteasome system.

The loss of a functional CFTR channel at the plasma membrane disrupts ionic homeostasis and airway surface hydration leading to reduced lung function. Reduced periciliary liquid volume and increased mucus viscosity impede mucociliary clearance resulting in chronic infection and inflammation. In the lung, the loss of CFTR-function leads to numerous physiological effects downstream of altered anion conductance that result in the dysfunction of additional organs such as the pancreas, intestine and gall bladder.

By studying the mechanistic aspects of CFTR misfolding and corrections, small molecules have been identified as CF modulators, that can act as stabilizers.

Despite the identification of compounds that modulate CFTR, there is no cure for this fatal disease and identification of new compounds and new methods of therapy are needed as well as new methods for treating or lessening the severity of cystic fibrosis and other CFTR mediated conditions and diseases in a patient.

### SUMMARY

The present disclosure includes a compound of formula A:

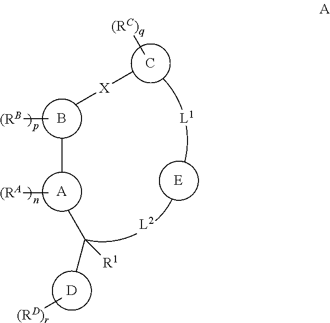

or a pharmaceutically acceptable salt thereof. Additionally, the present disclosure includes, among other things, pharmaceutical compositions, methods of using and methods of making a compound of formula A.

### DETAILED DESCRIPTION

In some embodiments, the present disclosure includes a compound of Formula A:

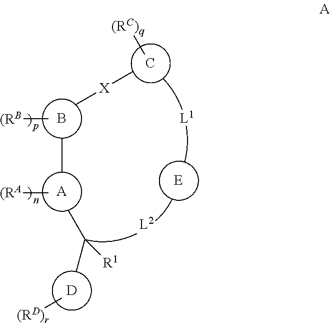

or a pharmaceutically acceptable salt thereof,
   wherein
L₁ is an optionally substituted C₁₋₆ alkylene chain wherein 1-3 of the methylene units is optionally and independently replaced by —O—, —N(R²)—, —C(O)—, —S—, —S(O)—, an optionally substituted 3-6 membered carbocyclyl,

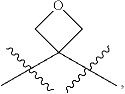 [SMILES: *C1(*)COC1]

optionally substituted C₂ alkenylene, or optionally substituted 5-6-membered heteroaryl;
L² is an optionally substituted C₁₋₆ alkylene chain wherein 1-3 of the methylene units is optionally and independently replaced by —C(CD₃)₂-, —O—, —N(R²)—, —C(O)—, —S—, —S(O)—, — an optionally substituted 3-6 membered carbocyclyl, optionally substituted C₂ alkenylene, or optionally substituted 5-6-membered heteroaryl;
Ring A is optionally substituted 5-membered heteroaryl comprising 1-3 heteroatoms selected from the group consisting of N, O or S;
Ring B is optionally substituted phenyl or optionally substituted 6-membered heteroaryl;
Ring C is optionally substituted phenyl or optionally substituted 5-10-membered heteroaryl
Ring D is optionally substituted phenyl or optionally substituted 5-6-membered heteroaryl;
Ring E is optionally substituted 5-6-membered heteroaryl comprising 1-4 heteroatoms selected from the group consisting of N, O or S;
X is selected from the group consisting of —O—, —S—, —CH₂—, —C(OH)H—, —SO—, —CO—, —SO₂—, —CFH—, —CF₂—, and —N(R²)—;
each R^{A} is independently selected from the group consisting of halogen, cyano, optionally substituted C₁-C₆ aliphatic, optionally substituted C₁-C₆ alkoxy, and —CD₃;
each R^{B} is independently selected from the group consisting of halogen, cyano, —C(O)N(R²)₂, C(O)OR², —OR², —N(R²)₂, optionally substituted C₁-C₆ aliphatic and optionally substituted C₁-C₆ alkoxy;
each R^{C} is independently selected from the group consisting of halogen, cyano, optionally substituted C₁-C₆ aliphatic or optionally substituted C₁-C₆ alkoxy;
each R^{D} is independently selected from the group consisting of halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S, wherein each R^{D} is optionally substituted with 1-6 instances of R^{d};
each R^{d} is independently selected from the group consisting of hydrogen, —OH, —CD₃, —C(O)N(R²)₂, C(O)OR², —OR², —N(R²)₂, optionally substituted C₁-C₆ aliphatic, optionally substituted 5-6-membered heteroaryl, and optionally substituted 3-6-membered heterocyclyl comprising 1-3 heteroatoms selected from the group consisting of N, O or S
R¹ is selected from the group consisting of hydrogen, cyano, —OR², —(CH₂)₀₋₃N(R²)₂, optionally substituted C₁-C₃ aliphatic, and —CD₃;
each R² is independently selected from hydrogen, optionally substituted C₁-C₆ aliphatic, —OH, C₁-C₆ alkoxy, —S(O)₂ (optionally substituted C₁-C₆ aliphatic);
n is 0, 1, 2 or 3;
p is 0, 1, 2, 3 or 4;
q is 0, 1 or 2; and
r is 0, 1, 2, 3, 4 or 5.

In some embodiments, the present disclosure includes a compound of Formula I:

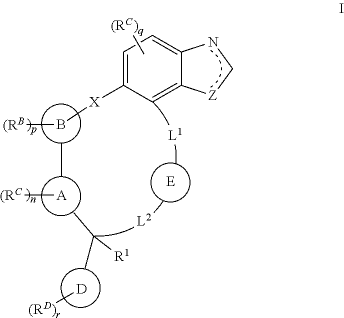

or a pharmaceutically acceptable salt thereof,
   wherein
L¹ is an optionally substituted C₁₋₆ alkylene chain wherein 1-3 of the methylene units is optionally and independently replaced by —O—, —N(R²)—, —C(O)—, —S—, —S(O)—, an optionally substituted 3-6 membered carbocyclyl,

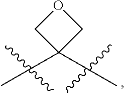 [SMILES: *C1(*)COC1]

optionally substituted C₂ alkenylene, or optionally substituted 5-6-membered heteroaryl;
L² is an optionally substituted C₁₋₆ alkylene chain wherein 1-3 of the methylene units is optionally and independently replaced by —C(CD₃)₂-, —O—, —N(R²)—, —C(O)—, —S—, —S(O)—, an optionally substituted 3-6 membered carbocyclyl, optionally substituted C₂ alkenylene, or optionally substituted 5-6-membered heteroaryl;
Ring A is a optionally substituted 5-membered heteroaryl comprising 1-4 heteroatoms selected from the group consisting of N, O or S;
Ring B is optionally substituted phenyl or optionally substituted 6-membered heteroaryl;
Ring D is optionally substituted phenyl or optionally substituted 5-6-membered heteroaryl;
Ring E is a optionally substituted 5-6-membered heteroaryl comprising 1-4 heteroatoms selected from the group consisting of N, O or S;
X is selected from the group consisting of —O—, —S—, —CH₂—, —C(OH)H—, —SO—, —CO—, —SO₂—, —CFH—, —CF₂—, and —N(R²)—;
each R^{A} is independently selected from the group consisting of halogen, cyano, optionally substituted C₁-C₆ aliphatic, optionally substituted C₁-C₆ alkoxy, and —CD₃;
each R^{B} is independently selected from the group consisting of halogen, cyano, —C(O)N(R²)₂, C(O)OR², —OR², —N(R²)₂, optionally substituted C₁-C₆ aliphatic and optionally substituted C₁-C₆ alkoxy;
each R^{C} is independently selected from the group consisting of hydrogen, halogen, cyano, optionally substituted C₁-C₆ aliphatic or optionally substituted C₁-C₆ alkoxy;
each R^{D} is independently selected from the group consisting of halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S, wherein each R^{D} is optionally substituted with 1-6 instances of R^{d};
   wherein two instances of R^{D} may be taken together to form an optionally substituted 5-7 membered carbocyclic ring, optionally substituted 5-6-membered heteroaryl, and optionally substituted 3-6-membered heterocyclyl comprising 1-3 heteroatoms selected from the group consisting of N, O or S;
each R^{d} is independently selected from the group consisting of hydrogen, —OH, —CD₃, —C(O)N(R²)₂, C(O)OR², —OR², —N(R²)₂, —S(O)₂R² optionally substituted C₁-C₆ aliphatic, optionally substituted 5-6-membered heteroaryl, and optionally substituted 3-6-membered heterocyclyl comprising 1-3 heteroatoms selected from the group consisting of N, O or S;
R¹ is selected from the group consisting of hydrogen, cyano, —OR², —(CH₂)₀₋₃N(R²)₂, optionally substituted C₁-C₃ aliphatic, 3-6-membered heterocyclyl comprising 1-3 heteroatoms selected from the group consisting of N, O or S, and —CD₃;
each R² is independently selected from hydrogen, optionally substituted C₁-C₆ aliphatic, —OH, C₁-C₆ alkoxy, —S(O)₂ (optionally substituted C₁-C₆ aliphatic);
Z is —CH═, —N═ or —NH—;
n is 0, 1, 2 or 3;
p is 0, 1, 2, 3 or 4;
q is 1 or 2; and
r is 0, 1, 2, 3, 4 or 5.

In some embodiments, the present disclosure includes a compound of formula I-a, I-b, I-c, or I-d:

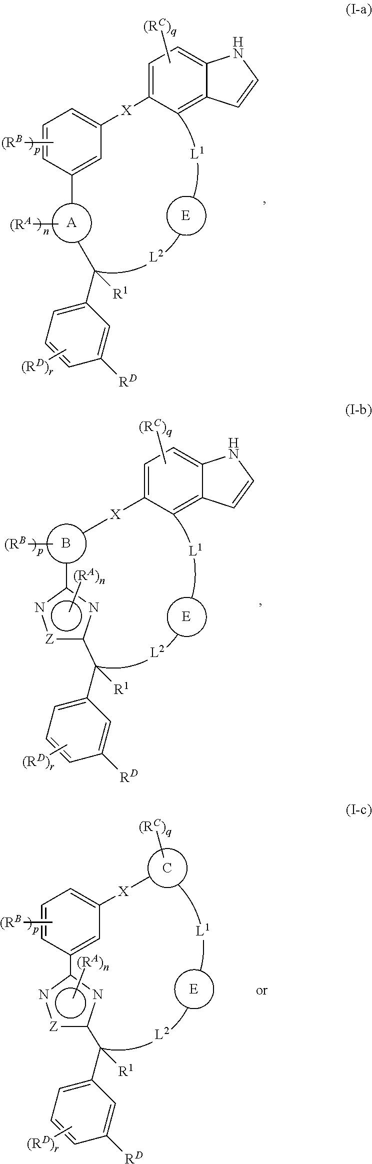

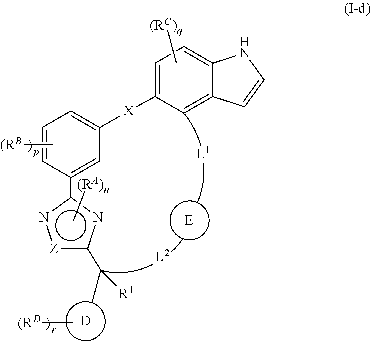

or a pharmaceutically acceptable salt thereof,
wherein Ring A, Ring B, Ring C, Ring D, Ring E, L¹, L², X, Z, R¹, R^{A}, R^{B}, R^{C}, R^{D}, n, p, q, and r are defined herein.

In some embodiments, the present disclosure includes a compound of formula (I-a1), (I-a2), (I-a3), (I-a4), or (I-a5):

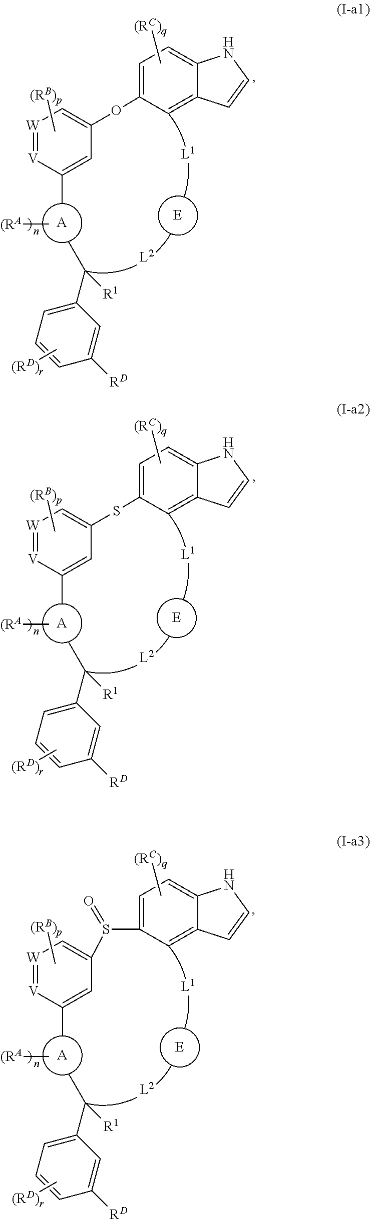

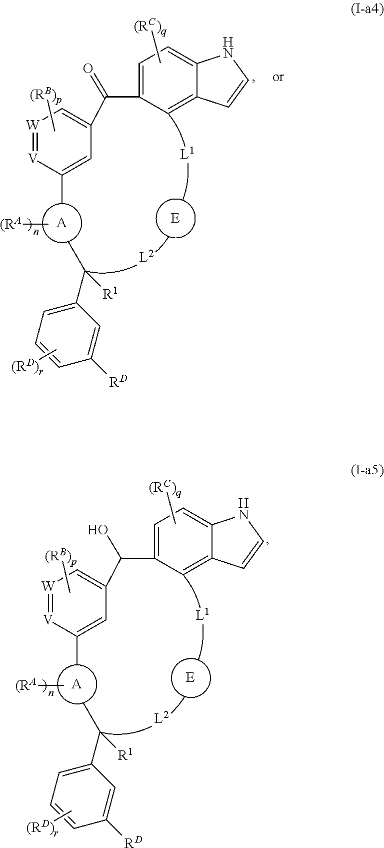

or a pharmaceutically acceptable salt thereof.
wherein Ring A, Ring E, L¹, L², W, V, R¹, R^{A}, R^{B}, R^{C}, R^{D}, n, p, q, and r are defined herein.

In some embodiments, the present disclosure includes a compound of formula (I-d1), (I-d2), (I-d3), (I-d4), or (I-d5)

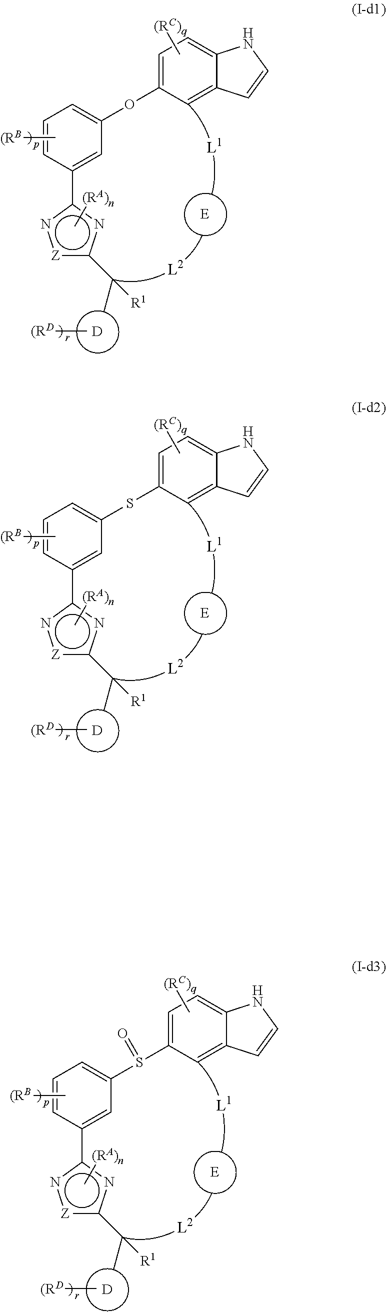

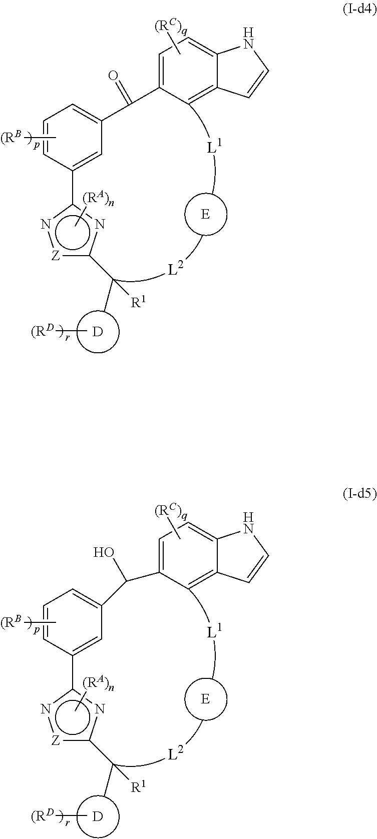

or a pharmaceutically acceptable salt thereof,
   wherein Ring D, Ring E, L¹, L², Z, R¹, R^{A}, R^{B}, R^{C}, R^{D}, n, p, q, and r are defined herein.

In some embodiments, the present disclosure includes compound of formula (I-e):

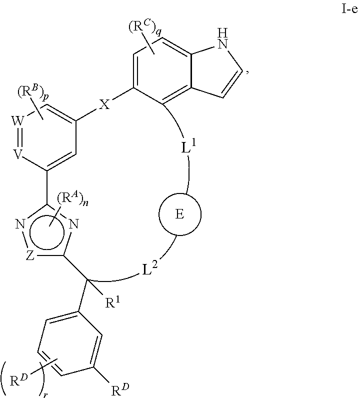

or a pharmaceutically acceptable salt thereof,
   wherein Ring E, L¹, L², V, W, X, Z, R¹, R^{A}, R^{B}, R^{C}, R^{D}, m, n, p, q, and r are defined herein.

In some embodiments, the present disclosure includes a compound of formula I-f or (I-f′):

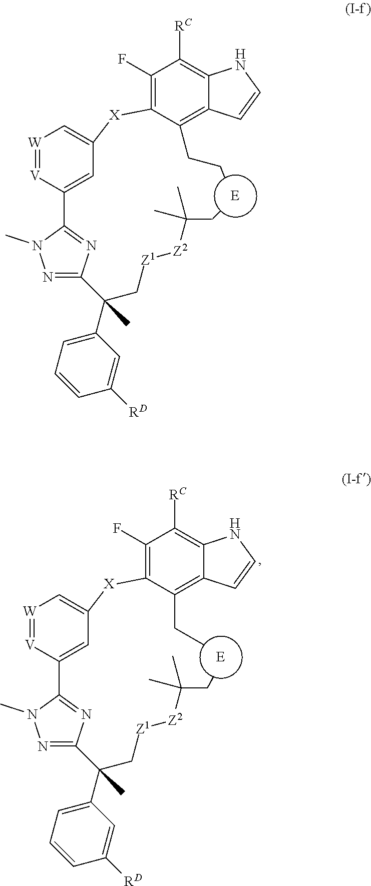

or a pharmaceutically acceptable salt thereof,
   wherein Ring E, V, W, X, Z¹, Z², R^{C}, and R^{D} are defined herein.

In some embodiments, the present disclosure includes a compound of formula I-g or (I-h):

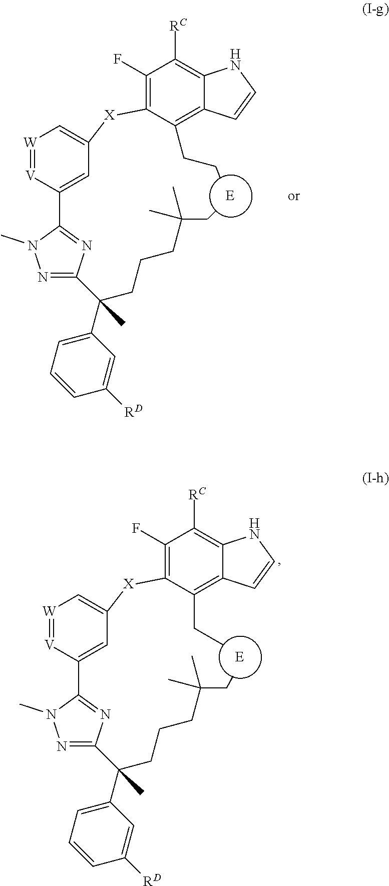

or a pharmaceutically acceptable salt thereof,
   wherein Ring E, V, W, X, R^{C}, and R^{D} are defined herein.

In some embodiments, the present disclosure includes a compound of formula (I-g1), (I-g2), (I-h1), or (I-h2):

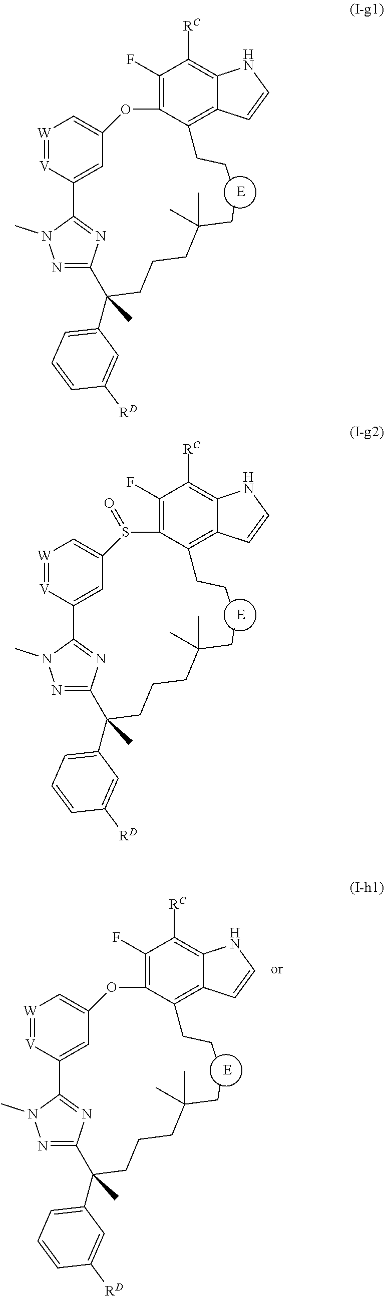

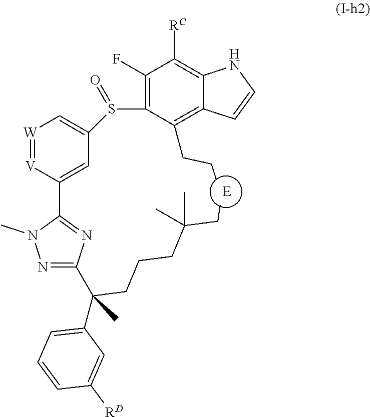

or a pharmaceutically acceptable salt thereof, wherein Ring E, V, W, R^{C}, and R^{D} are defined herein.

In some embodiments, the present disclosure includes a compound of formula I-i or I-j:

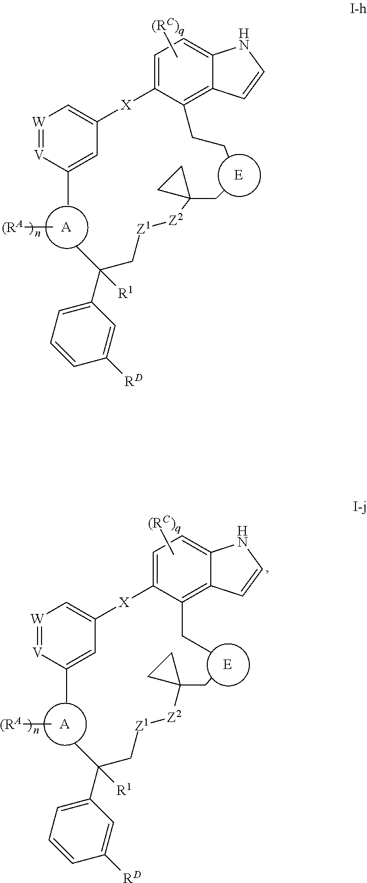

or a pharmaceutically acceptable salt thereof,
   wherein Ring A, Ring E, W, V, X, Z¹, Z², R¹, R^{A}, R^{C}, R^{D}, n, and q, are defined herein.

In some embodiments, the present disclosure includes a compound of formula I-k or I-l:

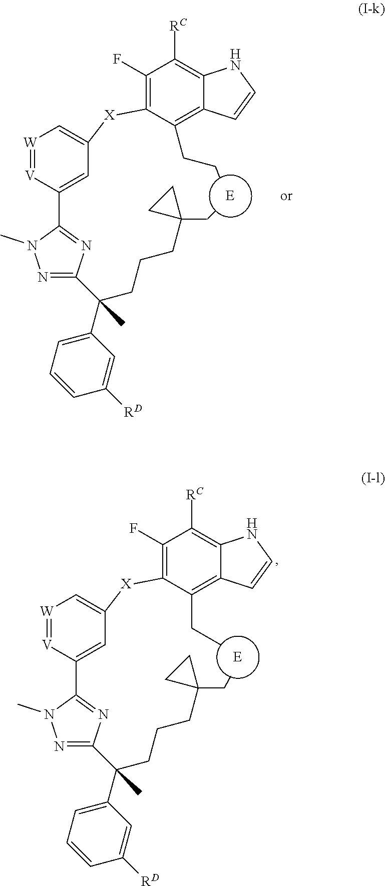

or a pharmaceutically acceptable salt thereof,
   wherein Ring E, V, W, X, R¹, R^{C}, and R^{D} are defined herein.

### Ring A

In some embodiments, Ring A is an optionally substituted 5-membered heteroaryl comprising 1-4 heteroatoms selected from the group consisting of N, O or S. In some embodiments, Ring A is an optionally substituted 5-membered heteroaryl comprising 1-3 heteroatoms selected from the group consisting of N and O. In some embodiments, Ring A is an optionally substituted 5-membered heteroaryl comprising 1 nitrogen atom. In some embodiments, Ring A is an optionally substituted 5-membered heteroaryl comprising 2 nitrogen atoms. In some embodiments, Ring A is an optionally substituted 5-membered heteroaryl comprising 3 nitrogen atoms.

In some embodiments, Ring A is selected from the group consisting of furan, pyrrole, thiophene, pyrazole, oxazole, thiazole, imidazole, triazole, tetrazole, oxadiazole, and thiadiazole. In some embodiments, Ring A is selected from the group consisting of imidazole, pyrazole, and triazole. In some embodiments, Ring A is selected from the group consisting of imidazole and triazole.

In some embodiments, Ring A is

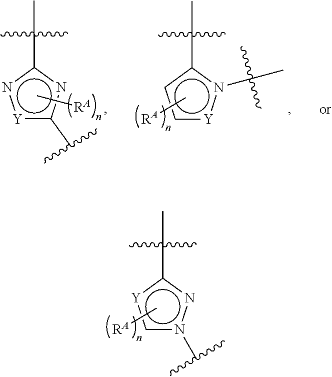

wherein Y is C or N.

In some embodiments, Ring A is selected from the group consisting of

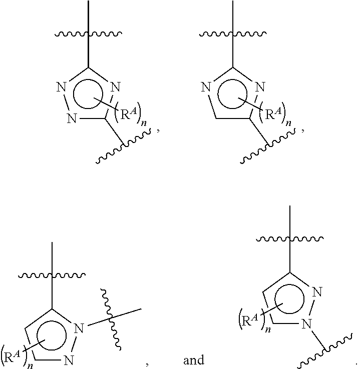

In some embodiments, Ring A is selected from the group consisting of

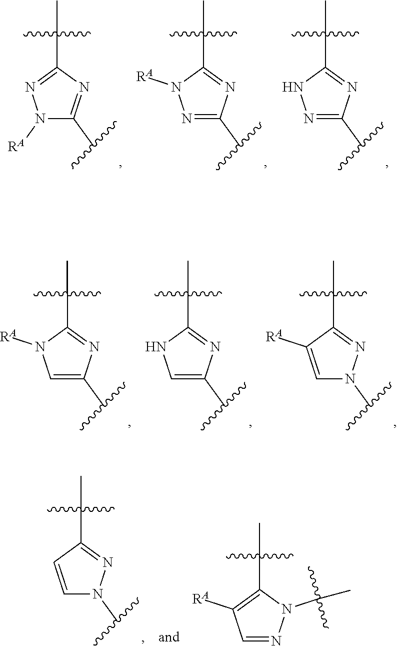

### Ring B

In some embodiments, Ring B is optionally substituted phenyl or optionally substituted 6-membered heteroaryl. In some embodiments, Ring B is optionally substituted phenyl, optionally substituted pyridine, or optionally substituted pyridone. In some embodiments, Ring B is optionally substituted phenyl. In some embodiments, Ring B is optionally substituted pyridyl. In some embodiments, Ring B is optionally substituted pyridone.

In some embodiments, Ring B is

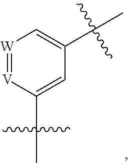

wherein
W is —CΠ=, —C(R^{B})═ or —N═; and
V is —CH═, —C(R^{B})═ or —N═.

In some embodiments, Ring B is selected from the group consisting of

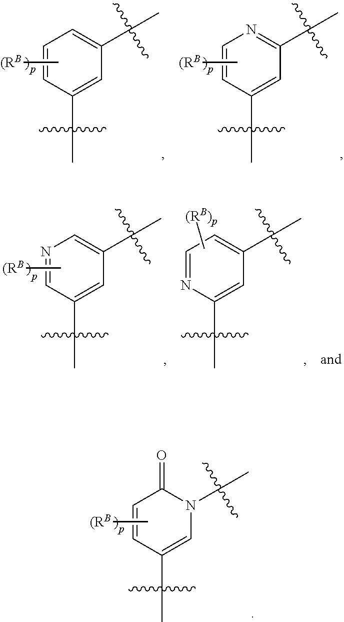

In some embodiments, Ring B is selected from the group consisting of

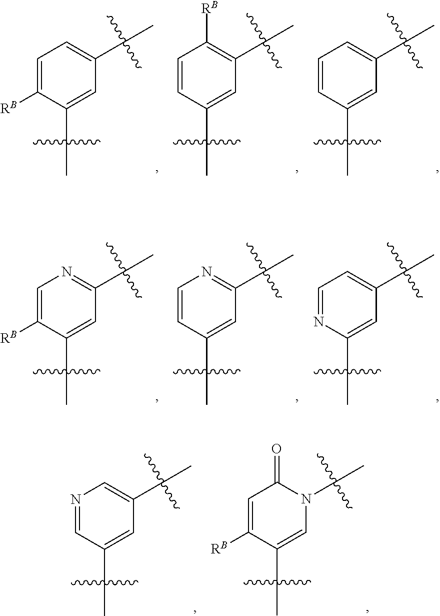

and

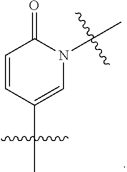 [SMILES: *c1ccc(=O)n(*)c1]

In some embodiments, Ring B is

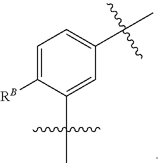

In some embodiments, Ring B is

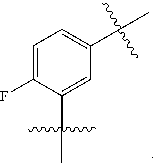 [SMILES: *c1ccc(F)c(*)c1]

### Ring C

In some embodiments, Ring C is optionally substituted 5-10-membered heteroaryl comprising 1-3 heteroatoms selected from the group consisting of O, S, and N. In some embodiments, Ring C is optionally substituted 9-10-membered heteroaryl comprising 1-3 heteroatoms selected from the group consisting of O, S, and N. In some embodiments, Ring C is selected from the group consisting of optionally substituted indole, optionally substituted indazole, optionally substituted benzimidazole, optionally substituted 6-azaindole, and optionally substituted 7-azaindole. In some embodiments, Ring C is optionally substituted indole.

In some embodiments, Ring C is

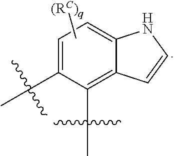

In some embodiments, Ring C is

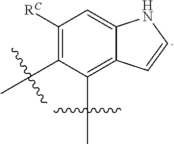

In some embodiments, Ring C is

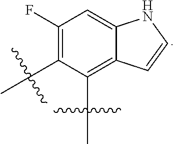 [SMILES: *c1c(F)cc2[nH]ccc2c1*]

In some embodiments, Ring C is

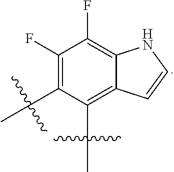 [SMILES: *c1c(F)c(F)c2[nH]ccc2c1*]

In some embodiments, Ring C is

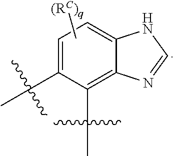

In some embodiments, Ring C is

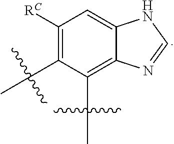

In some embodiments, Ring C is

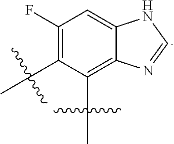 [SMILES: *c1c(F)cc2[nH]cnc2c1*]

### Ring D

In some embodiments, Ring D is optionally substituted phenyl or optionally substituted 5-6-membered heteroaryl comprising 1-3 heteroatoms selected from the group consisting of O, S, and N. In some embodiments, Ring D is optionally substituted phenyl. In some embodiments, Ring D is optionally substituted 5-6-membered heteroaryl comprising 1-3 heteroatoms selected from the group consisting of O, S, and N. In some embodiments, Ring D is optionally substituted 5-membered heteroaryl comprising 1-3 heteroatoms selected from the group consisting of O, S, and N. In some embodiments, Ring D is 6-membered heteroaryl comprising 1-3 heteroatoms selected from the group consisting of O, S, and N. In some embodiments Ring D is optionally substituted pyridine.

In some embodiments, Ring D is

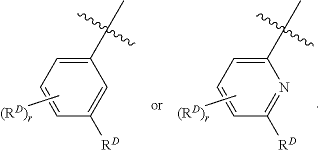

In some embodiments, Ring D is

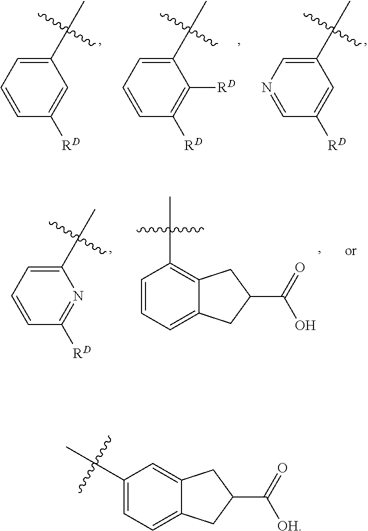

In some embodiments, Ring D is

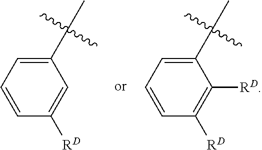

In some embodiments, Ring D is

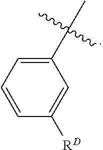

In some embodiments, Ring D is

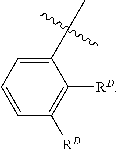

In some embodiments, Ring D is

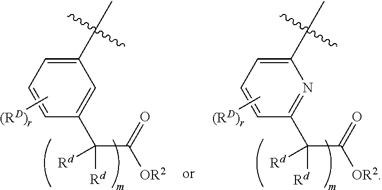

In some embodiments, Ring D is

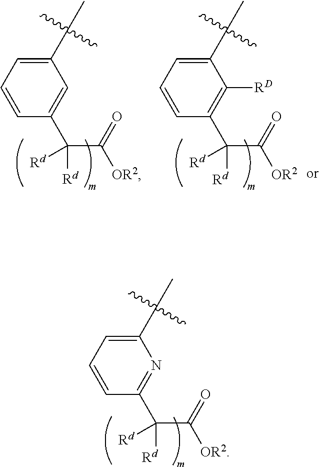

In some embodiments, Ring D is

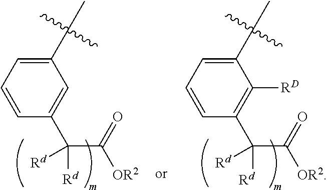

In some embodiments, Ring D is

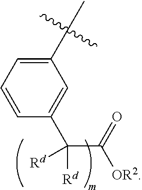

In some embodiments, Ring D is

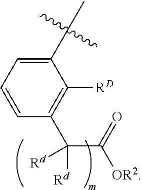

### Ring E

In some embodiments, Ring E is an optionally substituted 5, 6-membered heteroaryl comprising 1-4 heteroatoms selected from the group consisting of N, O or S. In some embodiments, Ring E is an optionally substituted 5, 6-membered heteroaryl comprising 1-3 heteroatoms selected from the group consisting of N and O. In some embodiments, Ring E is an optionally substituted 5, 6-membered heteroaryl comprising 1 nitrogen atom. In some embodiments, Ring E is an optionally substituted 5, 6-membered heteroaryl comprising 2 nitrogen atoms. In some embodiments, Ring E is an optionally substituted 5-membered heteroaryl comprising 3 nitrogen atoms.

In some embodiments, Ring E is selected from the group consisting of furan, pyrrole, thiophene, pyrazole, oxazole, thiazole, imidazole, triazole, tetrazole, oxadiazole, and thiadiazole. In some embodiments, Ring E is selected from the group consisting of pyrazole, oxazole, thiazole, imidazole, triazole, and tetrazole. In some embodiments, Ring E is selected from the group consisting of oxazole, pyrazole, and triazole. In some embodiments, Ring E is triazole.

L¹ and L²

In some embodiments, L¹ is an optionally substituted C₁₋₆ alkylene chain wherein 1-3 of the methylene units is optionally and independently replaced by —O—, —N(R²)—, —C(O)—, —S—, —S(O)—, an optionally substituted 3-6 membered carbocyclyl,

In some embodiments, Ring D is

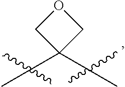 [SMILES: *C1(*)COC1]

optionally substituted C₂ alkenylene, or optionally substituted 5-6-membered heteroaryl. In some embodiments, L² is an optionally substituted C₁₋₆ alkylene chain wherein 1-3 of the methylene units is optionally and independently replaced by —C(CD₃)₂-, —O—, —N(R²), —C(O)—, —S—, —S(O)—, an optionally substituted 3-6 membered carbocyclyl, optionally substituted C₂ alkenylene, or optionally substituted 5-6-membered heteroaryl. In some embodiments, L¹ is an optionally substituted C₁₋₆ alkylene chain wherein 1-3 of the methylene units is optionally and independently replaced by —O—, —N(R²)—, —C(O)—,

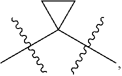 [SMILES: *C1(*)CC1]

or optionally substituted 5-6-membered heteroaryl, and L² is an optionally substituted C₁₋₆ alkylene chain wherein 1-3 of the methylene units is optionally and independently replaced by —C(CD₃)₂-, —O—, —N(R²)—, —C(O)—,

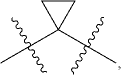 [SMILES: *C1(*)CC1]

or optionally substituted 5-6-membered heteroaryl. In some embodiments, L¹ is an optionally substituted C₁₋₆ alkylene chain and L² is an optionally substituted C₁₋₆ alkylene chain, wherein one of the methylene units of L² is optionally replaced with —O—. In some embodiments, L¹ is a C₁₋₆ alkylene chain substituted with 1-3 instances of methyl, and L² is C₁₋₆ alkylene chain, wherein one of the methylene units of L² is optionally replaced with —O— and wherein L² is optionally substituted with 1-3 instances of methyl. In some embodiments, L¹ is an unsubstituted C₂ alkylene chain. In some embodiments, L² is a C₅ alkylene chain, wherein one of the methylene units of L² is optionally replaced with —O— and wherein L² is optionally substituted with 1-3 instances of methyl. In some embodiments, L² is a C₅ alkylene chain, wherein L² is optionally substituted with 1-3 instances of methyl. In some embodiments, L² is optionally substituted with 1-3 instances of methyl.

In some embodiments, L¹ is

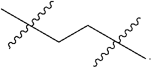 [SMILES: *CC*]

In some embodiments, L² is

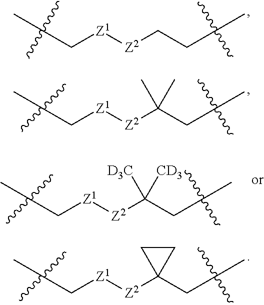

wherein Z¹ is —CH₂—, —CF₂—, —C(O)—, or —O—; and
Z² is —CH₂—, —CF₂—, —C(O)—, or —O—.

In some embodiments, L² is

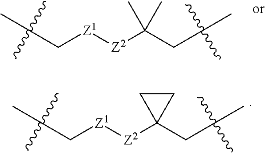

wherein Z¹ is —CH₂— or —O—; and
Z² is —CH₂— or —O—.

In some embodiments, L² is

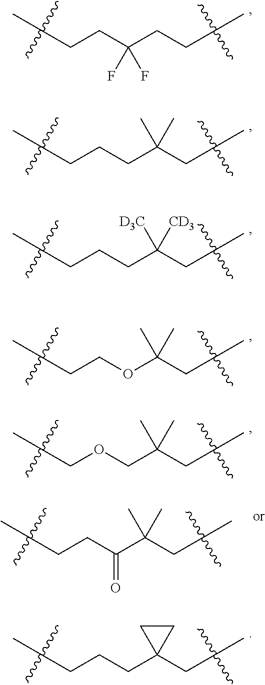

In some embodiments, L² is

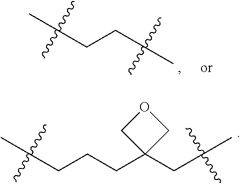

In some embodiments, Z¹ is —CH₂—, and Z² is —O—. In some embodiments, Z¹ is —O—, and Z¹ is —CH₂—.

R^{A}

In some embodiments, each R^{A} is independently selected from the group consisting of halogen, cyano, optionally substituted C₁-C₆ aliphatic, optionally substituted C₁-C₆ alkoxy, and —CD₃. In some embodiments, each R^{A} is independently selected from cyano and optionally substituted C₁-C₆ aliphatic. In some embodiments, each R^{A} is independently selected from cyano and optionally substituted C₁-C₃ aliphatic. In some embodiments, each R^{A} is independently optionally substituted C₁-C₃ aliphatic. In some embodiments, R^{A} is methyl.

### R^{B}

In some embodiments, each R^{B} is independently selected from the group consisting of hydrogen, halogen, cyano, —C(O)N(R²)₂, C(O)OR², —OR², —N(R²)₂, optionally substituted C₁-C₆ aliphatic and optionally substituted C₁-C₆ alkoxy. In some embodiments, each R^{B} is independently selected from halogen and cyano. In some embodiments, each R^{B} is independently selected from the group consisting of halogen and optionally substituted C₁-C₃ alkyl. In some embodiments, each R^{B} is independently selected from halogen. In some embodiments, R^{B} is fluoro.

### R^{C}

In some embodiments, each R^{C} is independently selected from the group consisting of hydrogen, halogen, cyano, optionally substituted C₁-C₆ aliphatic or optionally substituted C₁-C₆ alkoxy. In some embodiments, each R^{c} is independently selected from halogen, cyano, and optionally substituted C₁-C₆ alkyl. In some embodiments, each R^{C} is independently selected from halogen. In some embodiments, R^{C} is fluoro.

### R^{D}

In some embodiments, each R^{D} is independently selected from the group consisting of hydrogen, halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S, wherein each R^{D} is optionally substituted with 1-6 instances of R^{d};
wherein two instances of R^{D} may be taken together to form an optionally substituted 5-7 membered carbocyclic ring, optionally substituted 5-6-membered heteroaryl, and optionally substituted 3-6-membered heterocyclyl comprising 1-3 heteroatoms selected from the group consisting of N, O or S;

In some embodiments, each R^{D} is independently selected from the group consisting of hydrogen, halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S, wherein each R^{D} is optionally substituted with 1-6 instances of R^{d}.

In some embodiments, each R^{D} is independently selected from the group consisting of hydrogen, halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S.

In some embodiments, each R^{D} is independently selected from the group consisting of hydrogen, halogen, OR², and optionally substituted C₁-C₆ aliphatic. In some embodiments, each R^{D} is independently selected from the group consisting of halogen, OR², and optionally substituted C₁-C₆ aliphatic. In some embodiments, each R^{D} is independently selected from the group consisting of halogen, OR², optionally substituted C₁-C₃ alkyl, and optionally substituted C₂-C₃ alkenyl. In some embodiments, each R^{D} is independently selected from the group consisting of OR², optionally substituted C₁-C₃ alkyl, and optionally substituted C₂-C₃ alkenyl.

In some embodiments, each R^{D} is independently selected from hydrogen, halogen, —C(R^{d})₂OR²,

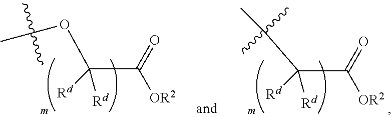

wherein
each R^{d} is independently hydrogen, optionally substituted methyl, —OH, —OMe, or —CD₃, wherein, two instances R^{d} may, with the atoms on which they are attached, form a cyclopropyl ring; and m is 0, 1, 2, or 3.

In some embodiments, r is 1 and R^{D} is —C(R^{d})₂OR² or

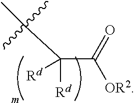

In some embodiments, r is 1 and R^{D} is —C(R^{d})₂OH or

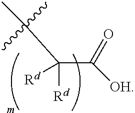

In some embodiments, R^{D} is selected from the group consisting of

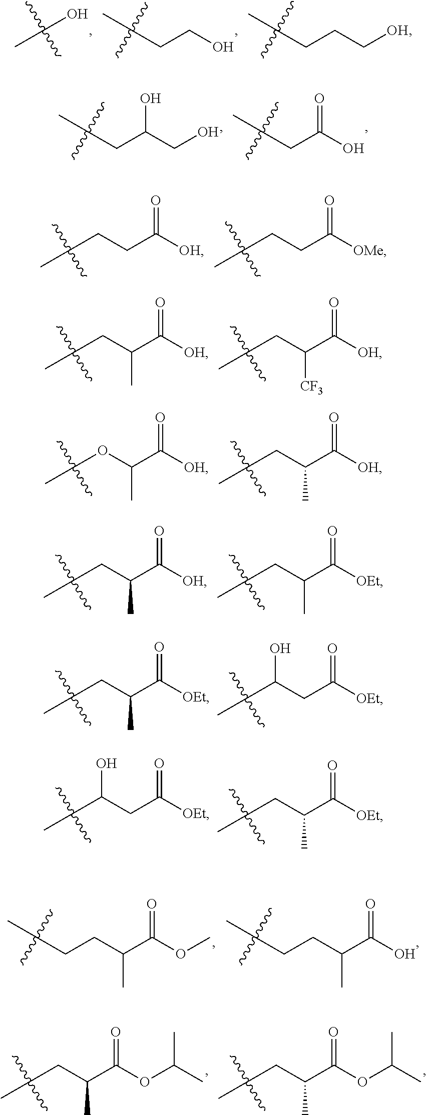

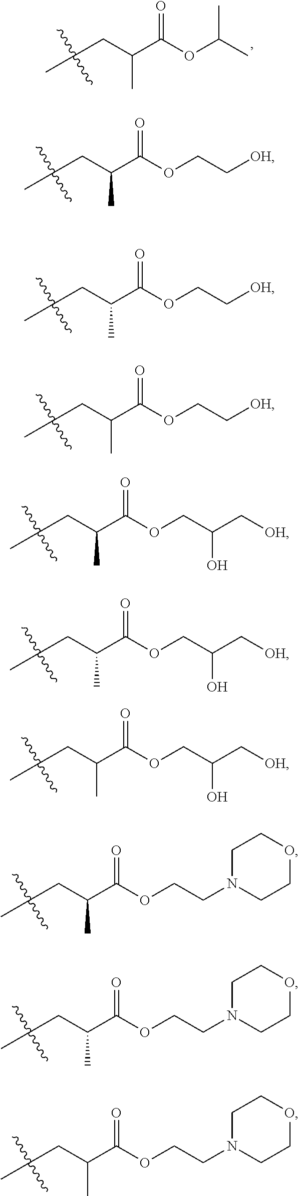

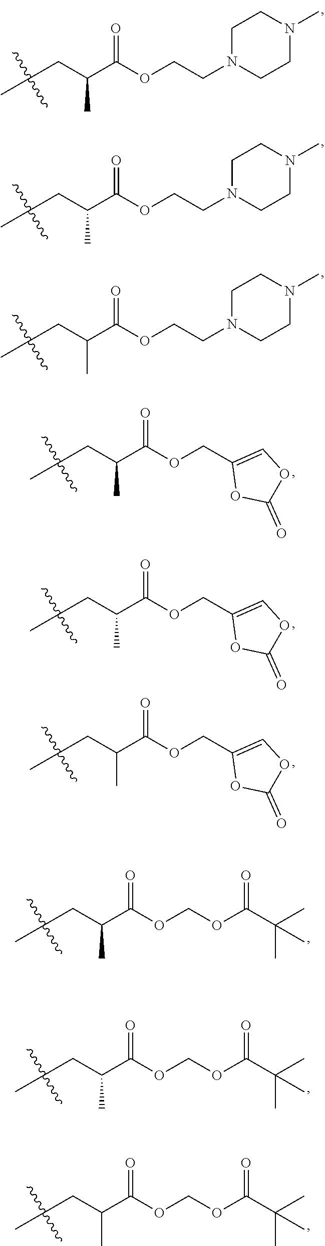

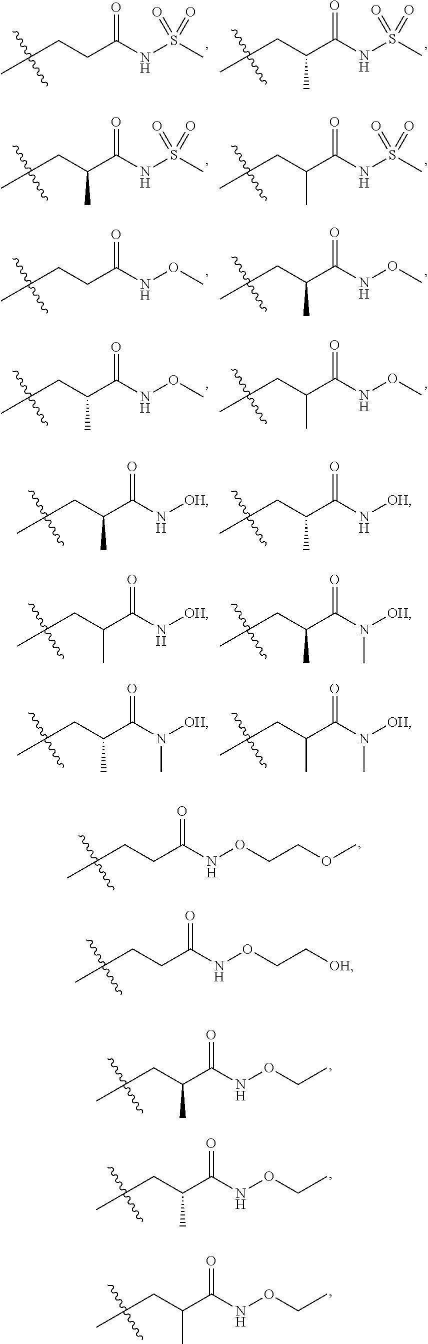

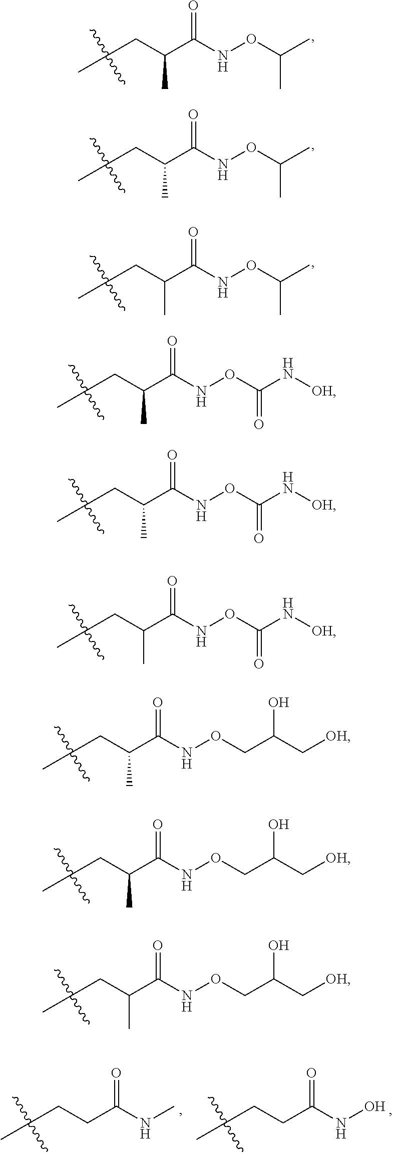

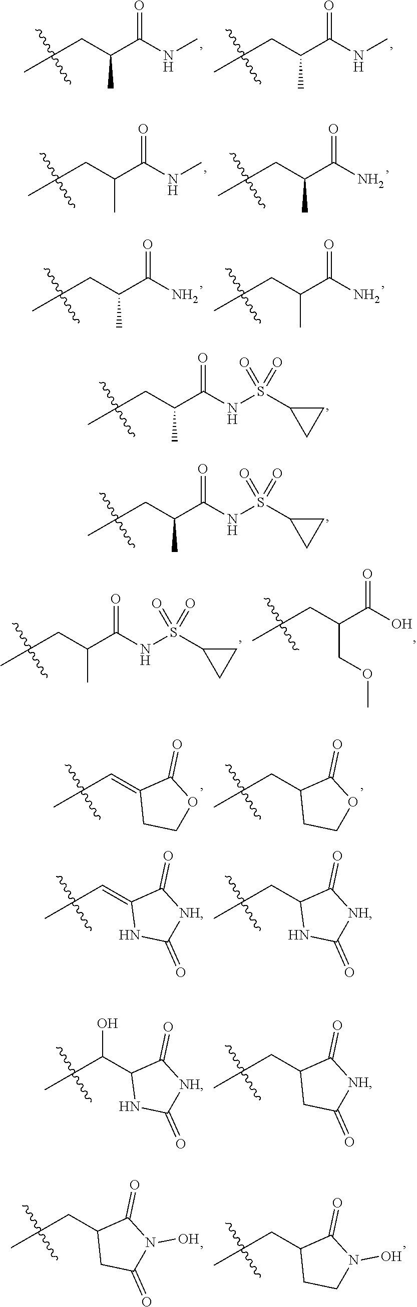

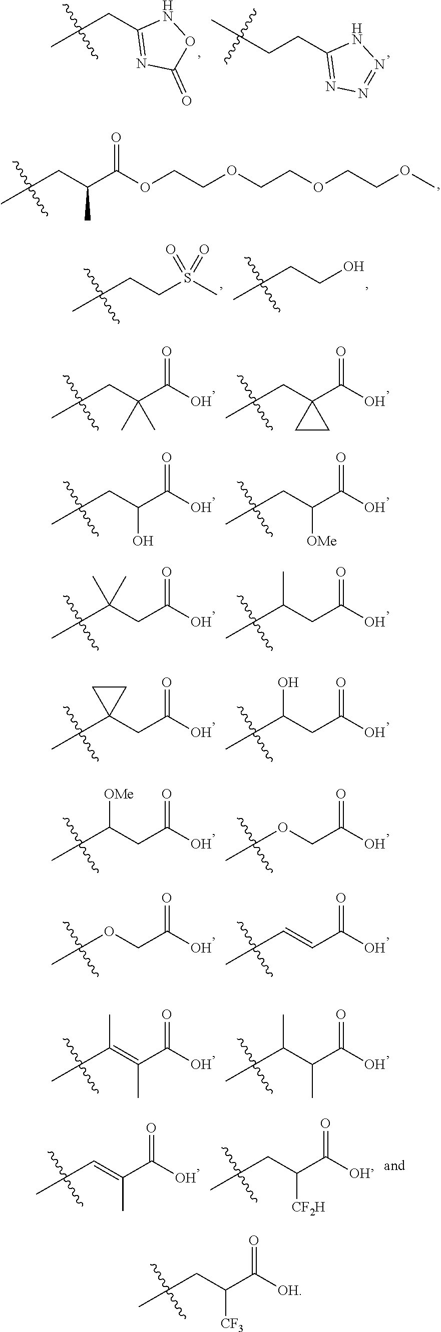

In some embodiments, R^{D} is selected from the group consisting of

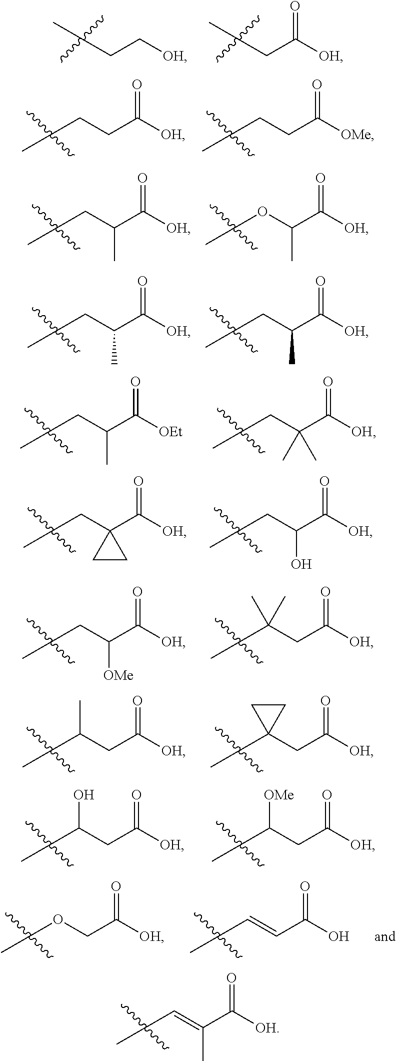

In some embodiments, R^{D} is selected from the group consisting of

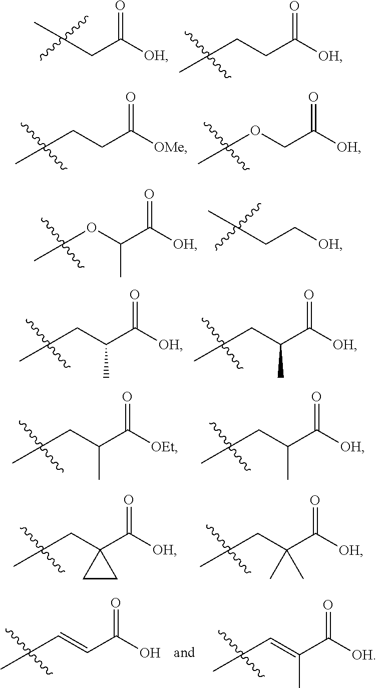

In some embodiments, R^{D} is selected from the group consisting of

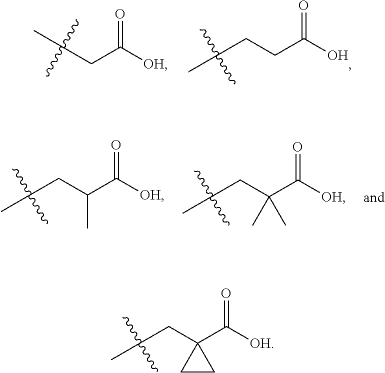

In some embodiments, R^{D} is

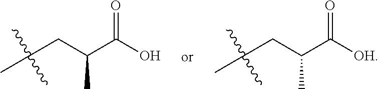

### R¹

In some embodiments, R¹ is selected from the group consisting of hydrogen, cyano, —OR², —(CH₂)₀₋₃N(R²)₂, optionally substituted C₁-C₃ aliphatic, 3-6-membered heterocyclyl comprising 1-3 heteroatoms selected from the group consisting of N, O or S, and —CD₃, In some embodiments, R¹ is selected from the group consisting of hydrogen, cyano, —OR², —(CH₂)₀₋₃N(R²)₂, optionally substituted C₁-C₃ aliphatic, and —CD₃. In some embodiments, R¹ is selected from the group consisting of hydrogen, cyano, and optionally substituted C₁-C₃ aliphatic. In some embodiments, R¹ is selected from the group consisting of hydrogen, cyano, optionally substituted methyl, and —CD₃. In some embodiments, R¹ is optionally substituted methyl. In some embodiments, R¹ is —CH₃. In some embodiments, R¹ is hydrogen. In some embodiments, R¹ is cyano. In some embodiments, R¹ is —CD₃. In some embodiments, R¹ is —CH₂NHCH₂CF₃. In some embodiments, R¹ is CH₂NH₂.

### R²

In some embodiments, each R² is independently selected from hydrogen, optionally substituted C₁-C₆ aliphatic, —OH, C₁-C₆ alkoxy, —S(O)₂ (optionally substituted C₁-C₆ aliphatic). In some embodiments, each R² is independently hydrogen or optionally substituted C₁-C₆ aliphatic. In some embodiments, each R² is independently hydrogen or optionally substituted C₁-C₃ aliphatic. In some embodiments, each R² is independently hydrogen or optionally substituted methyl. In some embodiments, R² is optionally substituted C₁-C₆ aliphatic. In some embodiments, R² is hydrogen. In some embodiments, each R² is independently optionally substituted methyl or optionally substituted ethyl. In some embodiments, each R² is independently optionally substituted methyl.

### R^{d}

In some embodiments, each R^{d} is independently selected from the group consisting of hydrogen, —OH, —CD₃, —C(O)N(R²)₂, C(O)OR², —OR², —N(R²)₂, optionally substituted C₁-C₆ aliphatic, optionally substituted 5-6-membered heteroaryl, and optionally substituted 3-6-membered heterocyclyl comprising 1-3 heteroatoms selected from the group consisting of N, O or S. In some embodiments, each R^{d} is independently selected from the group consisting of hydrogen, optionally substituted C₁₋₃ alkyl, —OH, —OMe, or —CD₃, wherein, two instances R^{d} may, with the atoms on which they are attached, form a cyclopropyl ring. In some embodiments, each R^{d} is independently selected from the group consisting of hydrogen, methyl, —CF₃, —CF₂H, or —CFH₂. In some embodiments, each R^{d} is independently selected from hydrogen and methyl. In some embodiments, R^{d} is hydrogen.

### X

In some embodiments, X is selected from the group consisting of —O—, —S—, —CH₂—, —C(OH)H—, —SO—, —CO—, —SO₂—, —CFH—, —CF₂—, and —N(R²)—. In some embodiments, X is selected from the group consisting of —O—, —S—, —CH₂—, —SO—, —CO—, —C(OH)H—, and —SO₂—. In some embodiments, X is —O—. In some embodiments, X is —S—. In some embodiments, X is —CH₂—. In some embodiments, X is —SO—. In some embodiments, X is —CO—. In some embodiments, X is —C(OH)H—. In some embodiments, X is —SO₂—. In some embodiments, X is

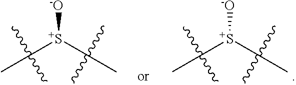

In some embodiments, X is

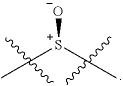

In some embodiments, X is

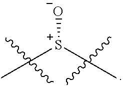

m, n, p, q, and r

In some embodiments, m is 0, 1, 2, or 3. In some embodiments, m is 1, 2, or 3. In some embodiments, m is 0. In some embodiments, m is 1. In some embodiments, m is 2. In some embodiments, m is 3.

In some embodiments, n is 0, 1, 2, or 3. In some embodiments, n is 1, 2, or 3. In some embodiments, n is 0. In some embodiments, n is 1. In some embodiments, n is 2. In some embodiments, n is 3.

In some embodiments, p is 0, 1, 2, 3, or 4. In some embodiments, p is 1, 2, 3, or 4. In some embodiments, p is 0. In some embodiments, p is 1. In some embodiments, p is 2. In some embodiments, p is 3. In some embodiments, p is 4.

In some embodiments, q is 0, 1, or 2. In some embodiments, q is 1 or 2. In some embodiments, q is 0. In some embodiments, q is 1. In some embodiments, q is 2.

In some embodiments, r is 0, 1, 2, 3, 4, or 5. In some embodiments, r is 1, 2, 3, or 4. In some embodiments, r is 0. In some embodiments, r is 1. In some embodiments, r is 2. In some embodiments, r is 3. In some embodiments, r is 4. In some embodiments, r is 5.

In some embodiments, the present disclosure includes compounds listed in Table 1.

TABLE 1

| Ex- | |
|---|---|
| am- | |
| ple | |
| No. | Structure |
| 1 | 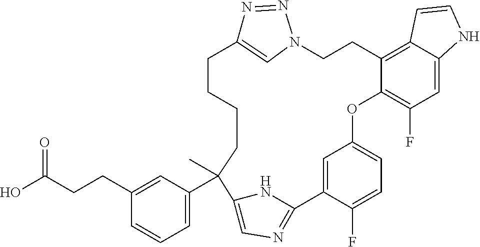 [SMILES: CC1(c2cccc(CCC(=O)O)c2)CCCCc2cn(nn2)CCc2c(c(F)cc3[nH]ccc23)Oc2ccc(F)c(c2)-c2ncc1[nH]2] |
| 2 | 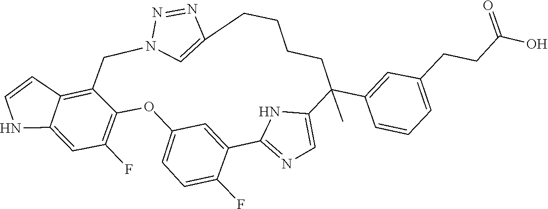 [SMILES: CC1(c2cccc(CCC(=O)O)c2)CCCCc2cn(nn2)Cc2c(c(F)cc3[nH]ccc23)Oc2ccc(F)c(c2)-c2ncc1[nH]2] |
| 3 | 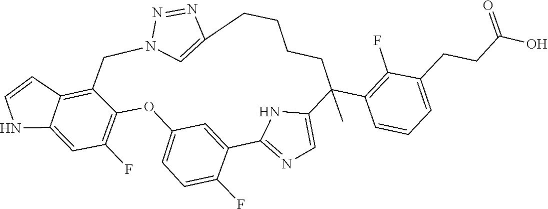 [SMILES: CC1(c2cccc(CCC(=O)O)c2F)CCCCc2cn(nn2)Cc2c(c(F)cc3[nH]ccc23)Oc2ccc(F)c(c2)-c2ncc1[nH]2] |
| 4 | 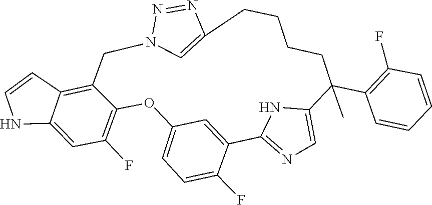 [SMILES: CC1(c2ccccc2F)CCCCc2cn(nn2)Cc2c(c(F)cc3[nH]ccc23)Oc2ccc(F)c(c2)-c2ncc1[nH]2] |
| 5 | 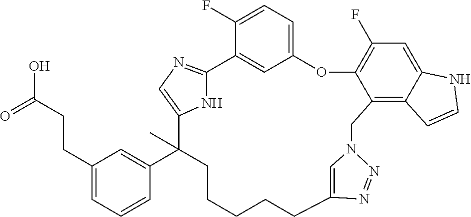 [SMILES: CC1(c2cccc(CCC(=O)O)c2)CCCCCc2cn(nn2)Cc2c(c(F)cc3[nH]ccc23)Oc2ccc(F)c(c2)-c2ncc1[nH]2] |
| 6 | 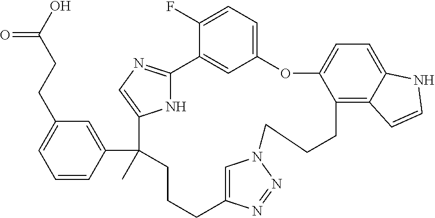 [SMILES: CC1(c2cccc(CCC(=O)O)c2)CCCc2cn(nn2)CCCc2c(ccc3[nH]ccc23)Oc2ccc(F)c(c2)-c2ncc1[nH]2] |
| 7 | 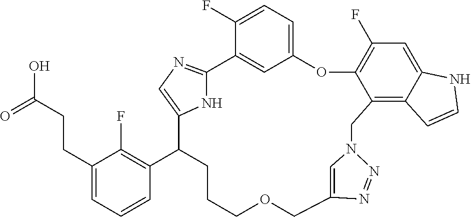 [SMILES: O=C(O)CCc1cccc(C2CCCOCc3cn(nn3)Cc3c(c(F)cc4[nH]ccc34)Oc3ccc(F)c(c3)-c3ncc2[nH]3)c1F] |
| 8A | 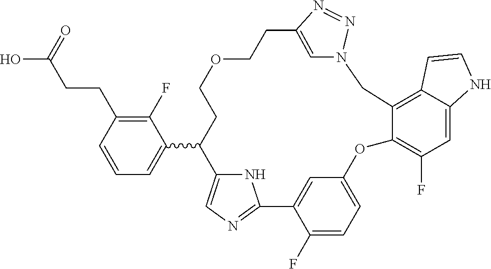 [SMILES: O=C(O)CCc1cccc(C2CCOCCc3cn(nn3)Cc3c(c(F)cc4[nH]ccc34)Oc3ccc(F)c(c3)-c3ncc2[nH]3)c1F] |
| 8B | 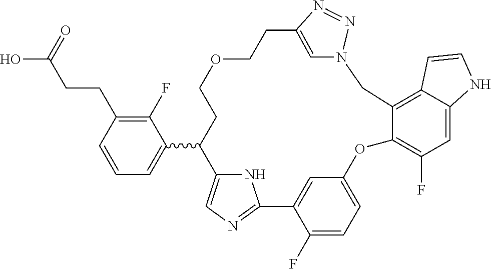 [SMILES: O=C(O)CCc1cccc(C2CCOCCc3cn(nn3)Cc3c(c(F)cc4[nH]ccc34)Oc3ccc(F)c(c3)-c3ncc2[nH]3)c1F] |
| 9 | 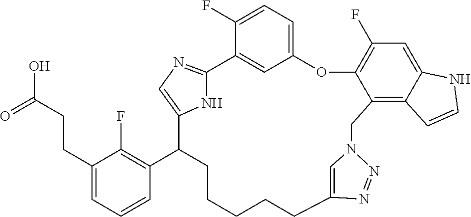 [SMILES: O=C(O)CCc1cccc(C2CCCCCc3cn(nn3)Cc3c(c(F)cc4[nH]ccc34)Oc3ccc(F)c(c3)-c3ncc2[nH]3)c1F] |
| 10 | 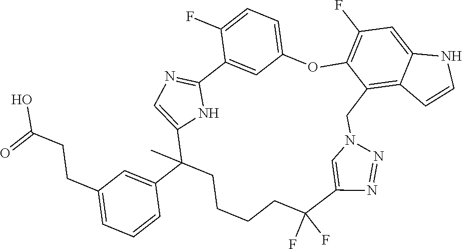 [SMILES: CC1(c2cccc(CCC(=O)O)c2)CCCCC(F)(F)c2cn(nn2)Cc2c(c(F)cc3[nH]ccc23)Oc2ccc(F)c(c2)-c2ncc1[nH]2] |
| 11 | 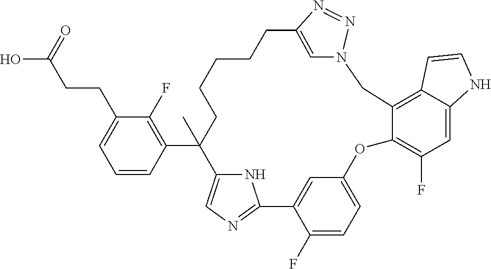 [SMILES: CC1(c2cccc(CCC(=O)O)c2F)CCCCCc2cn(nn2)Cc2c(c(F)cc3[nH]ccc23)Oc2ccc(F)c(c2)-c2ncc1[nH]2] |
| 12 | 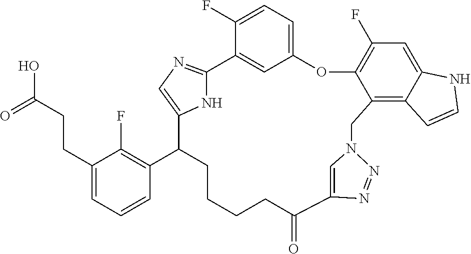 [SMILES: O=C(O)CCc1cccc(C2CCCCC(=O)c3cn(nn3)Cc3c(c(F)cc4[nH]ccc34)Oc3ccc(F)c(c3)-c3ncc2[nH]3)c1F] |
| 13 | 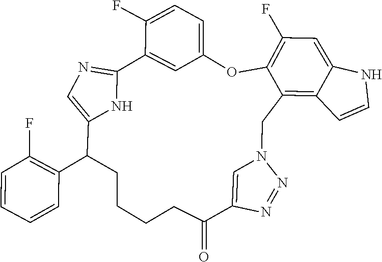 [SMILES: O=C1CCCCC(c2ccccc2F)c2cnc([nH]2)-c2cc(ccc2F)Oc2c(F)cc3[nH]ccc3c2Cn2cc1nn2] |
| 14 | 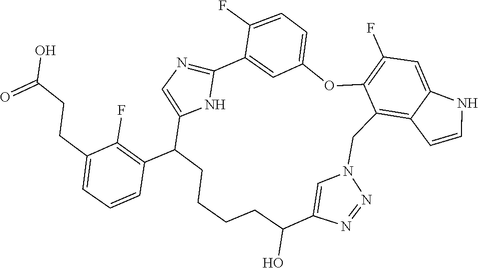 [SMILES: O=C(O)CCc1cccc(C2CCCCC(O)c3cn(nn3)Cc3c(c(F)cc4[nH]ccc34)Oc3ccc(F)c(c3)-c3ncc2[nH]3)c1F] |
| 15 | 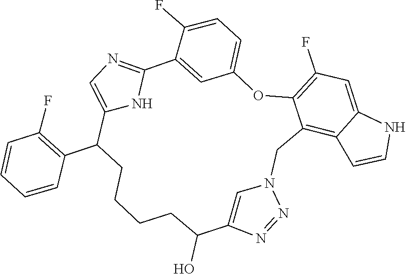 [SMILES: OC1CCCCC(c2ccccc2F)c2cnc([nH]2)-c2cc(ccc2F)Oc2c(F)cc3[nH]ccc3c2Cn2cc1nn2] |
| 16 | 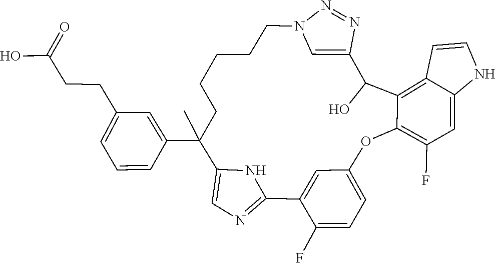 [SMILES: CC1(c2cccc(CCC(=O)O)c2)CCCCCn2cc(nn2)C(O)c2c(c(F)cc3[nH]ccc23)Oc2ccc(F)c(c2)-c2ncc1[nH]2] |
| 17 | 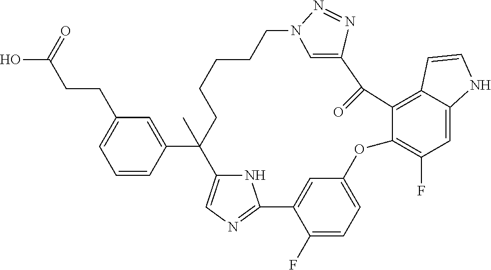 [SMILES: CC1(c2cccc(CCC(=O)O)c2)CCCCCn2cc(nn2)C(=O)c2c(c(F)cc3[nH]ccc23)Oc2ccc(F)c(c2)-c2ncc1[nH]2] |
| 18 | 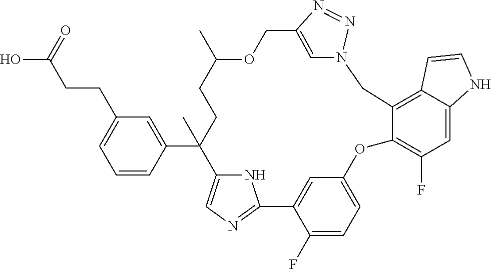 [SMILES: CC1CCC(C)(c2cccc(CCC(=O)O)c2)c2cnc([nH]2)-c2cc(ccc2F)Oc2c(F)cc3[nH]ccc3c2Cn2cc(nn2)CO1] |
| 19A | 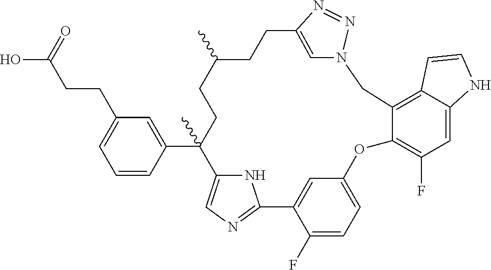 |
| 19B | 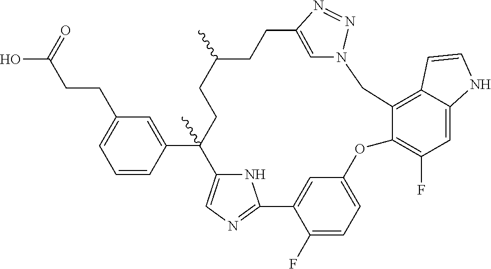 |
| 20A | 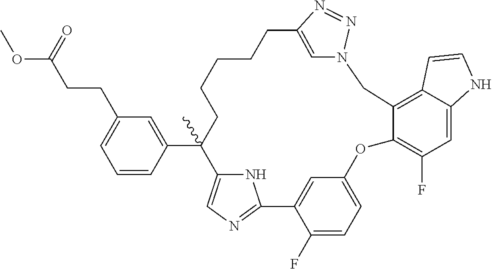 [SMILES: COC(=O)CCc1cccc(C2(C)CCCCCc3cn(nn3)Cc3c(c(F)cc4[nH]ccc34)Oc3ccc(F)c(c3)-c3ncc2[nH]3)c1] |
| 20B | 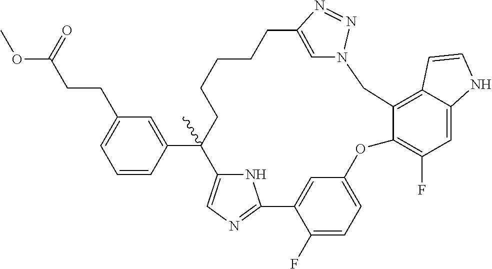 [SMILES: COC(=O)CCc1cccc(C2(C)CCCCCc3cn(nn3)Cc3c(c(F)cc4[nH]ccc34)Oc3ccc(F)c(c3)-c3ncc2[nH]3)c1] |
| 21 | 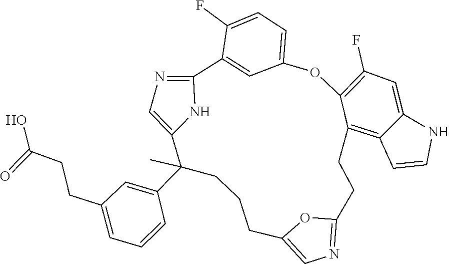 [SMILES: CC1(c2cccc(CCC(=O)O)c2)CCCC2=CN=C(CCc3c(c(F)cc4[nH]ccc34)Oc3ccc(F)c(c3)-c3ncc1[nH]3)O2] |
| 22 | 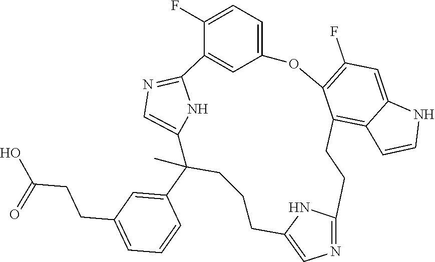 [SMILES: CC1(c2cccc(CCC(=O)O)c2)CCCc2cnc([nH]2)CCc2c(c(F)cc3[nH]ccc23)Oc2ccc(F)c(c2)-c2ncc1[nH]2] |
| 23 | 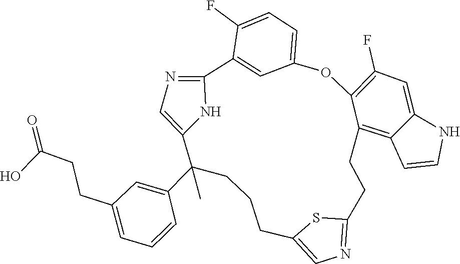 [SMILES: CC1(c2cccc(CCC(=O)O)c2)CCCC2=CN=C(CCc3c(c(F)cc4[nH]ccc34)Oc3ccc(F)c(c3)-c3ncc1[nH]3)S2] |
| 24 | 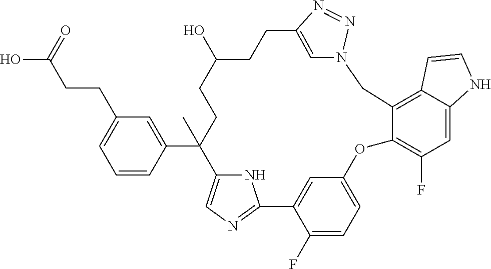 [SMILES: CC1(c2cccc(CCC(=O)O)c2)CCC(O)CCc2cn(nn2)Cc2c(c(F)cc3[nH]ccc23)Oc2ccc(F)c(c2)-c2ncc1[nH]2] |
| 25 | 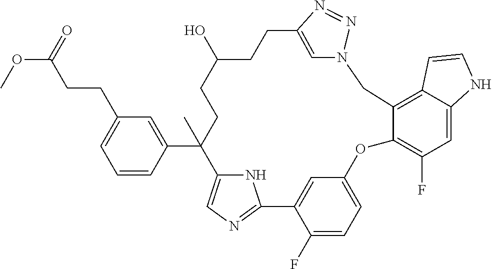 [SMILES: COC(=O)CCc1cccc(C2(C)CCC(O)CCc3cn(nn3)Cc3c(c(F)cc4[nH]ccc34)Oc3ccc(F)c(c3)-c3ncc2[nH]3)c1] |
| 26 | 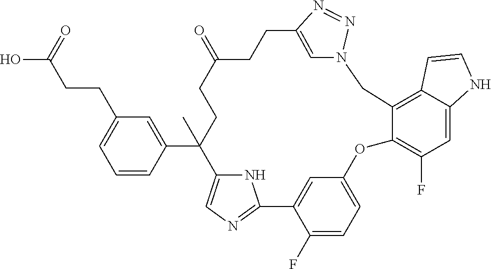 [SMILES: CC1(c2cccc(CCC(=O)O)c2)CCC(=O)CCc2cn(nn2)Cc2c(c(F)cc3[nH]ccc23)Oc2ccc(F)c(c2)-c2ncc1[nH]2] |
| 27A | 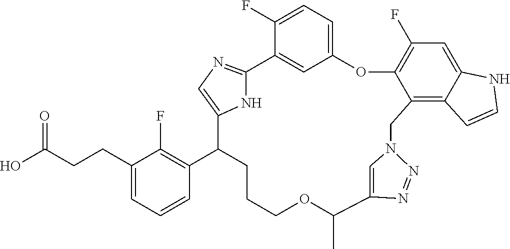 [SMILES: CC1OCCCC(c2cccc(CCC(=O)O)c2F)c2cnc([nH]2)-c2cc(ccc2F)Oc2c(F)cc3[nH]ccc3c2Cn2cc1nn2] |
| 27B | 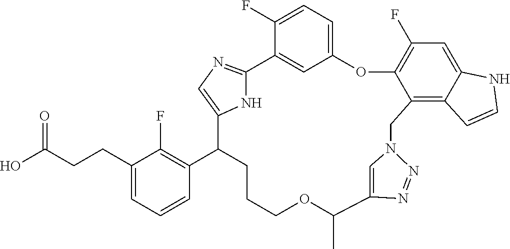 [SMILES: CC1OCCCC(c2cccc(CCC(=O)O)c2F)c2cnc([nH]2)-c2cc(ccc2F)Oc2c(F)cc3[nH]ccc3c2Cn2cc1nn2] |
| 27C | 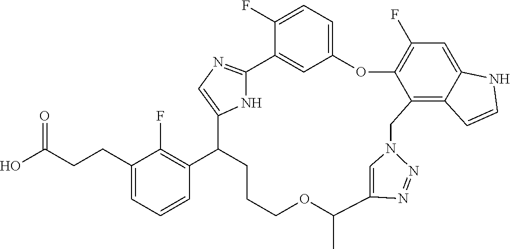 [SMILES: CC1OCCCC(c2cccc(CCC(=O)O)c2F)c2cnc([nH]2)-c2cc(ccc2F)Oc2c(F)cc3[nH]ccc3c2Cn2cc1nn2] |
| 28 | 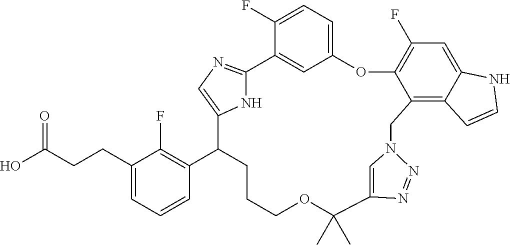 [SMILES: CC1(C)OCCCC(c2cccc(CCC(=O)O)c2F)c2cnc([nH]2)-c2cc(ccc2F)Oc2c(F)cc3[nH]ccc3c2Cn2cc1nn2] |
| 29A | 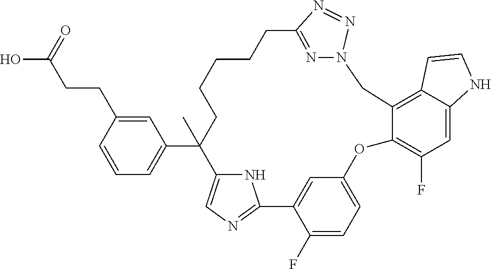 [SMILES: CC1(c2cccc(CCC(=O)O)c2)CCCCCc2nnn(n2)Cc2c(c(F)cc3[nH]ccc23)Oc2ccc(F)c(c2)-c2ncc1[nH]2] |
| 29B | 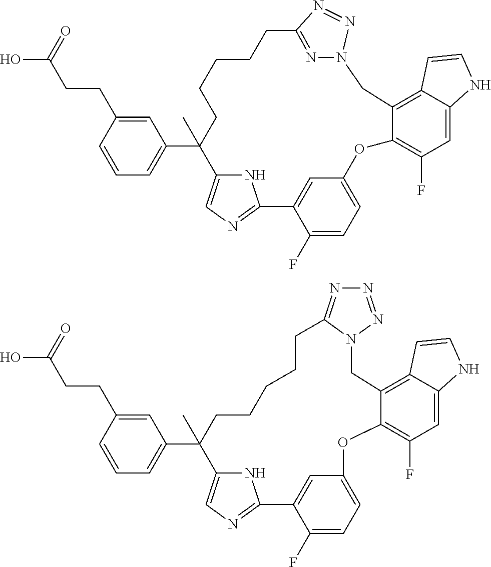 [SMILES: CC1(c2cccc(CCC(=O)O)c2)CCCCCc2nnn(n2)Cc2c(c(F)cc3[nH]ccc23)Oc2ccc(F)c(c2)-c2ncc1[nH]2 ; CC1(c2cccc(CCC(=O)O)c2)CCCCCc2nnnn2Cc2c(c(F)cc3[nH]ccc23)Oc2ccc(F)c(c2)-c2ncc1[nH]2] |
| 30A | 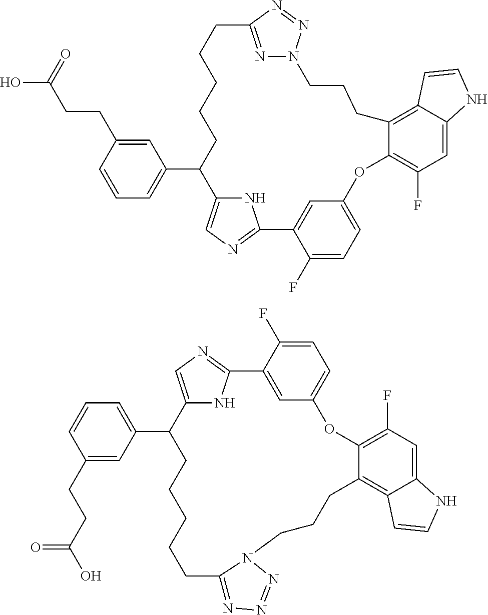 |
| 30B | 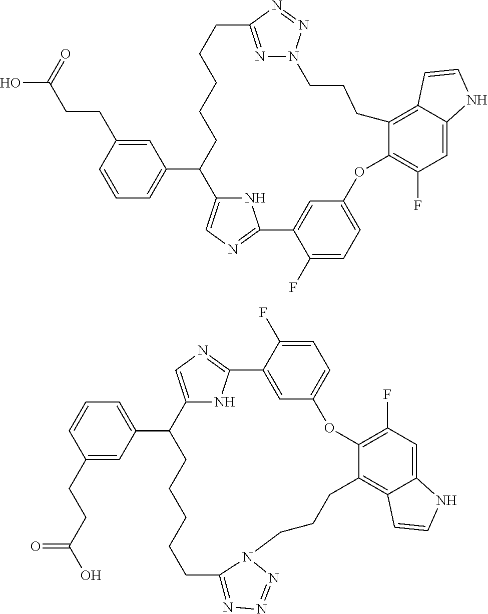 |
| 31 | 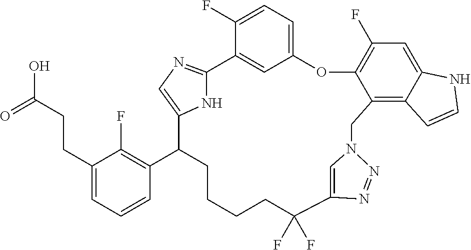 [SMILES: O=C(O)CCc1cccc(C2CCCCC(F)(F)c3cn(nn3)Cc3c(c(F)cc4[nH]ccc34)Oc3ccc(F)c(c3)-c3ncc2[nH]3)c1F] |
| 32 | 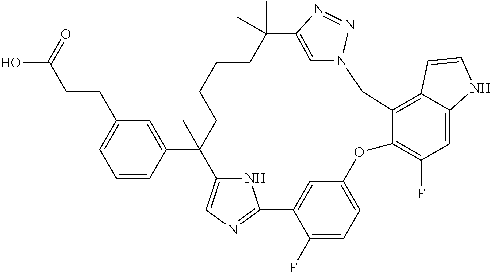 [SMILES: CC1(C)CCCCC(C)(c2cccc(CCC(=O)O)c2)c2cnc([nH]2)-c2cc(ccc2F)Oc2c(F)cc3[nH]ccc3c2Cn2cc1nn2] |
| 33 | 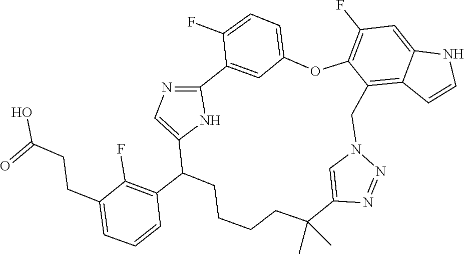 [SMILES: CC1(C)CCCCC(c2cccc(CCC(=O)O)c2F)c2cnc([nH]2)-c2cc(ccc2F)Oc2c(F)cc3[nH]ccc3c2Cn2cc1nn2] |
| 34 | 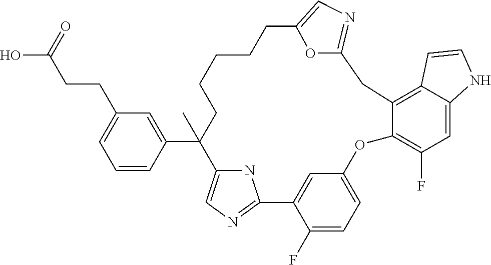 [SMILES: CC1(c2cccc(CCC(=O)O)c2)CCCCCC2=CN=C(Cc3c(c(F)cc4[nH]ccc34)Oc3ccc(F)c(c3)-c3ncc1[nH]3)O2] |
| 35 | 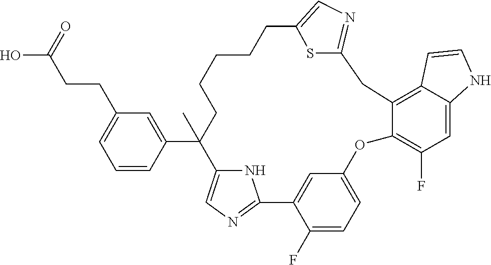 [SMILES: CC1(c2cccc(CCC(=O)O)c2)CCCCCC2=CN=C(Cc3c(c(F)cc4[nH]ccc34)Oc3ccc(F)c(c3)-c3ncc1[nH]3)S2] |
| 36 | 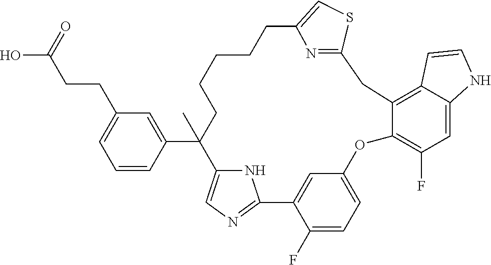 [SMILES: CC1(c2cccc(CCC(=O)O)c2)CCCCCc2csc(n2)Cc2c(c(F)cc3[nH]ccc23)Oc2ccc(F)c(c2)-c2ncc1[nH]2] |
| 37 | 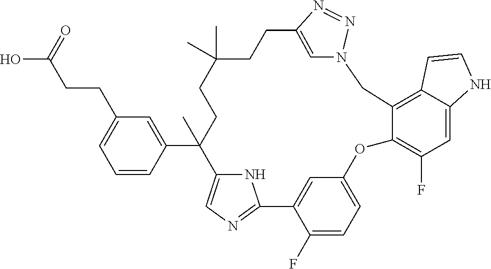 [SMILES: CC1(C)CCc2cn(nn2)Cc2c(c(F)cc3[nH]ccc23)Oc2ccc(F)c(c2)-c2ncc([nH]2)C(C)(c2cccc(CCC(=O)O)c2)CC1] |
| 38 | 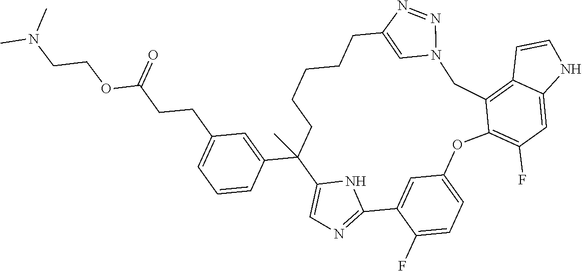 [SMILES: CN(C)CCOC(=O)CCc1cccc(C2(C)CCCCCc3cn(nn3)Cc3c(c(F)cc4[nH]ccc34)Oc3ccc(F)c(c3)-c3ncc2[nH]3)c1] |
| 39A | 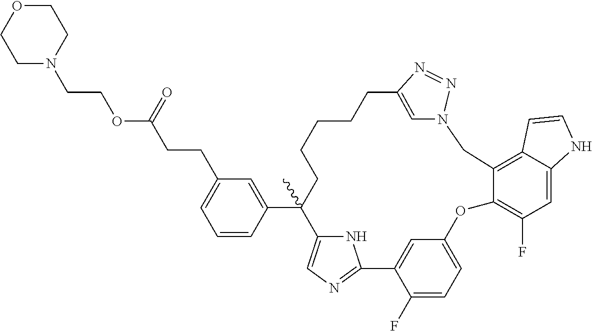 [SMILES: CC1(c2cccc(CCC(=O)OCCN3CCOCC3)c2)CCCCCc2cn(nn2)Cc2c(c(F)cc3[nH]ccc23)Oc2ccc(F)c(c2)-c2ncc1[nH]2] |
| 39B | 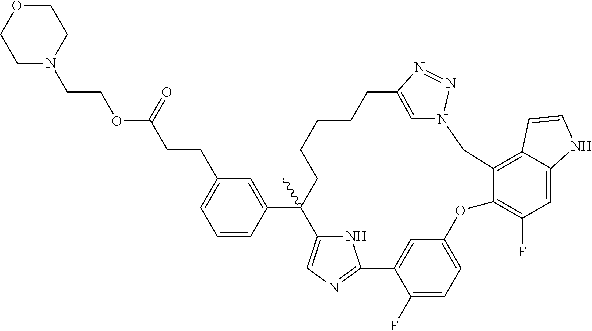 [SMILES: CC1(c2cccc(CCC(=O)OCCN3CCOCC3)c2)CCCCCc2cn(nn2)Cc2c(c(F)cc3[nH]ccc23)Oc2ccc(F)c(c2)-c2ncc1[nH]2] |
| 40 | 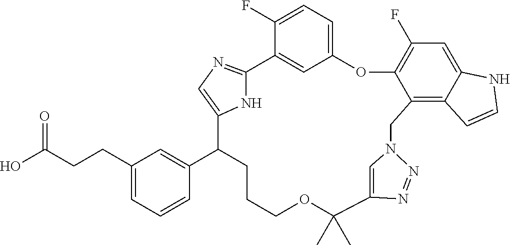 [SMILES: CC1(C)OCCCC(c2cccc(CCC(=O)O)c2)c2cnc([nH]2)-c2cc(ccc2F)Oc2c(F)cc3[nH]ccc3c2Cn2cc1nn2] |
| 41 | 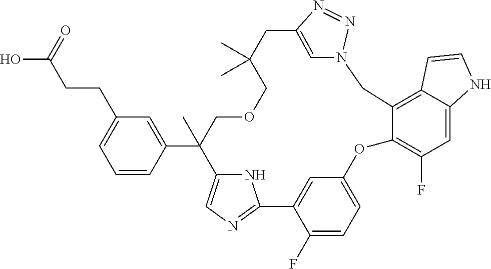 [SMILES: CC1(C)COCC(C)(c2cccc(CCC(=O)O)c2)c2cnc([nH]2)-c2cc(ccc2F)Oc2c(F)cc3[nH]ccc3c2Cn2cc(nn2)C1] |
| 42 | 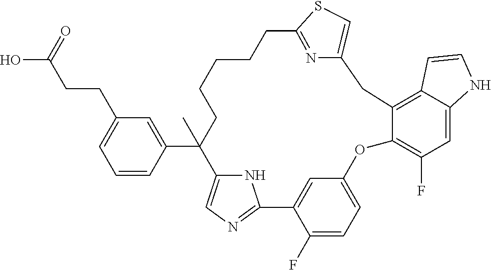 [SMILES: CC1(c2cccc(CCC(=O)O)c2)CCCCCc2nc(cs2)Cc2c(c(F)cc3[nH]ccc23)Oc2ccc(F)c(c2)-c2ncc1[nH]2] |
| 43 | 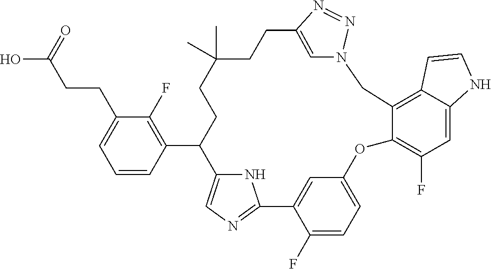 [SMILES: CC1(C)CCc2cn(nn2)Cc2c(c(F)cc3[nH]ccc23)Oc2ccc(F)c(c2)-c2ncc([nH]2)C(c2cccc(CCC(=O)O)c2F)CC1] |
| 44 | 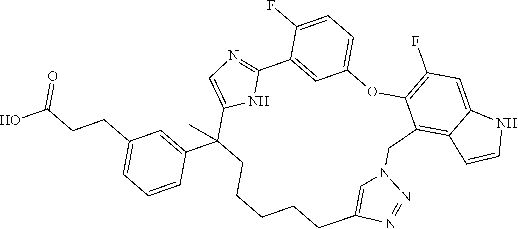 [SMILES: CC1(c2cccc(CCC(=O)O)c2)CCCCCc2cn(nn2)Cc2c(c(F)cc3[nH]ccc23)Oc2ccc(F)c(c2)-c2ncc1[nH]2] |
| 45A | 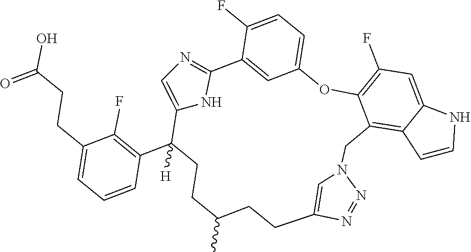 [SMILES: CC1CCc2cn(nn2)Cc2c(c(F)cc3[nH]ccc23)Oc2ccc(F)c(c2)-c2ncc([nH]2)C(c2cccc(CCC(=O)O)c2F)CC1] |
| 45B | 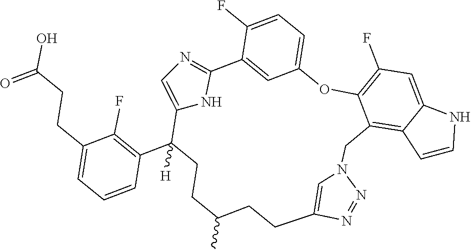 [SMILES: CC1CCc2cn(nn2)Cc2c(c(F)cc3[nH]ccc23)Oc2ccc(F)c(c2)-c2ncc([nH]2)C(c2cccc(CCC(=O)O)c2F)CC1] |
| 46 | 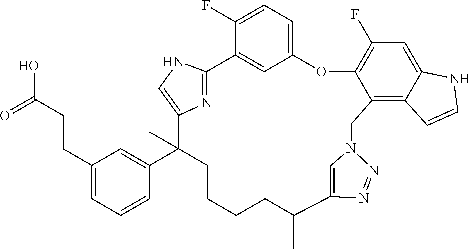 [SMILES: CC1CCCCC(C)(c2cccc(CCC(=O)O)c2)c2c[nH]c(n2)-c2cc(ccc2F)Oc2c(F)cc3[nH]ccc3c2Cn2cc1nn2] |
| 47 | 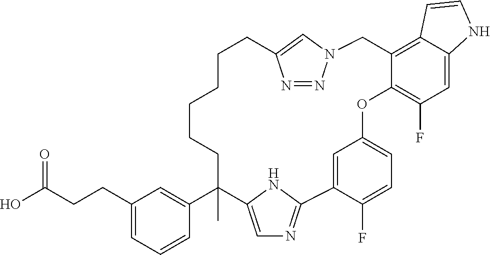 [SMILES: CC1(c2cccc(CCC(=O)O)c2)CCCCCCc2cn(nn2)Cc2c(c(F)cc3[nH]ccc23)Oc2ccc(F)c(c2)-c2ncc1[nH]2] |
| 48 | 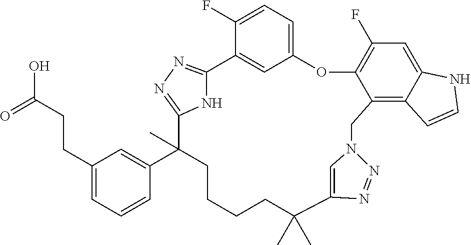 [SMILES: CC1(C)CCCCC(C)(c2cccc(CCC(=O)O)c2)c2nnc([nH]2)-c2cc(ccc2F)Oc2c(F)cc3[nH]ccc3c2Cn2cc1nn2] |
| 49 | 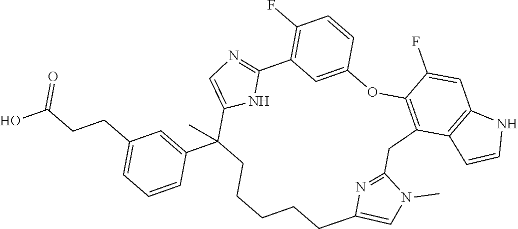 [SMILES: Cn1cc2nc1Cc1c(c(F)cc3[nH]ccc13)Oc1ccc(F)c(c1)-c1ncc([nH]1)C(C)(c1cccc(CCC(=O)O)c1)CCCCC2] |
| 50 | 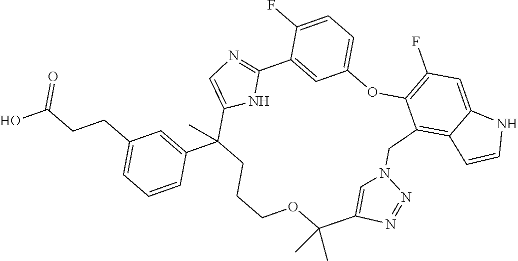 [SMILES: CC1(C)OCCCC(C)(c2cccc(CCC(=O)O)c2)c2cnc([nH]2)-c2cc(ccc2F)Oc2c(F)cc3[nH]ccc3c2Cn2cc1nn2] |
| 51 | 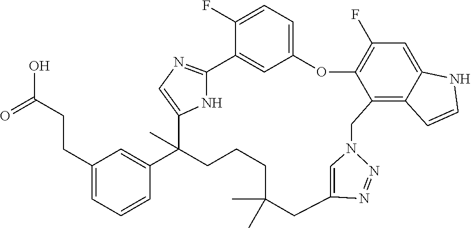 [SMILES: CC1(C)CCCC(C)(c2cccc(CCC(=O)O)c2)c2cnc([nH]2)-c2cc(ccc2F)Oc2c(F)cc3[nH]ccc3c2Cn2cc(nn2)C1] |
| 52 | 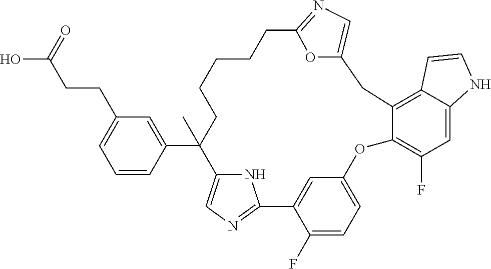 [SMILES: CC1(c2cccc(CCC(=O)O)c2)CCCCCC2=NC=C(Cc3c(c(F)cc4[nH]ccc34)Oc3ccc(F)c(c3)-c3ncc1[nH]3)O2] |
| 53 | 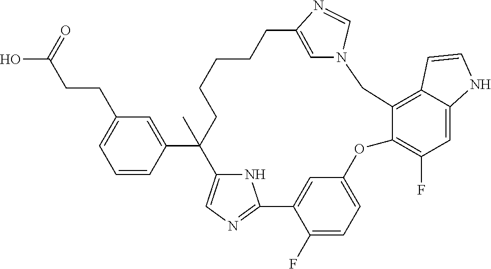 [SMILES: CC1(c2cccc(CCC(=O)O)c2)CCCCCc2cn(cn2)Cc2c(c(F)cc3[nH]ccc23)Oc2ccc(F)c(c2)-c2ncc1[nH]2] |
| 54 | 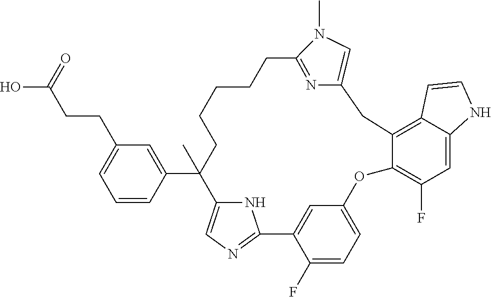 [SMILES: Cn1cc2nc1CCCCCC(C)(c1cccc(CCC(=O)O)c1)c1cnc([nH]1)-c1cc(ccc1F)Oc1c(F)cc3[nH]ccc3c1C2] |
| 55 | 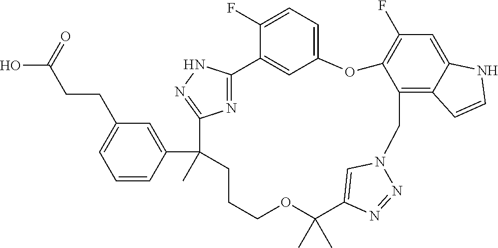 [SMILES: CC1(C)OCCCC(C)(c2cccc(CCC(=O)O)c2)c2n[nH]c(n2)-c2cc(ccc2F)Oc2c(F)cc3[nH]ccc3c2Cn2cc1nn2] |
| 56A | 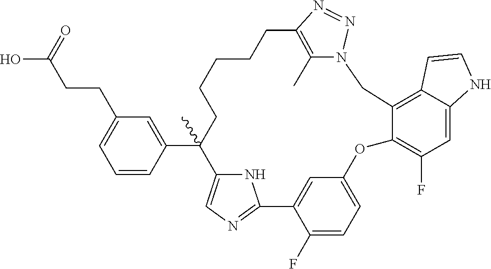 [SMILES: Cc1c2nnn1Cc1c(c(F)cc3[nH]ccc13)Oc1ccc(F)c(c1)-c1ncc([nH]1)C(C)(c1cccc(CCC(=O)O)c1)CCCCC2] |
| 56B | 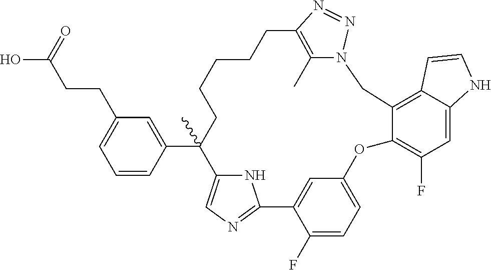 [SMILES: Cc1c2nnn1Cc1c(c(F)cc3[nH]ccc13)Oc1ccc(F)c(c1)-c1ncc([nH]1)C(C)(c1cccc(CCC(=O)O)c1)CCCCC2] |
| 57 | 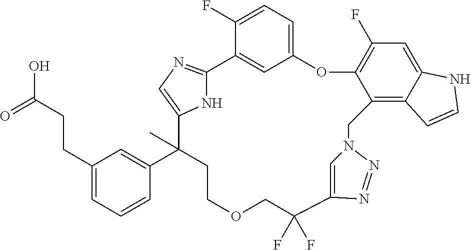 [SMILES: CC1(c2cccc(CCC(=O)O)c2)CCOCC(F)(F)c2cn(nn2)Cc2c(c(F)cc3[nH]ccc23)Oc2ccc(F)c(c2)-c2ncc1[nH]2] |
| 58A | 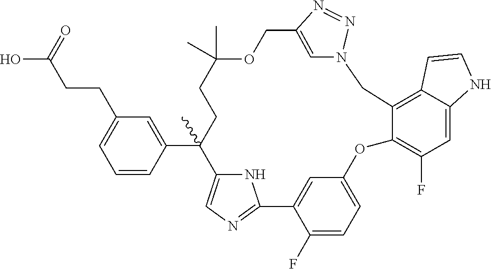 [SMILES: CC1(C)CCC(C)(c2cccc(CCC(=O)O)c2)c2cnc([nH]2)-c2cc(ccc2F)Oc2c(F)cc3[nH]ccc3c2Cn2cc(nn2)CO1] |
| 58B | 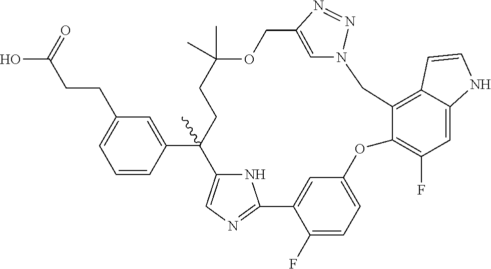 [SMILES: CC1(C)CCC(C)(c2cccc(CCC(=O)O)c2)c2cnc([nH]2)-c2cc(ccc2F)Oc2c(F)cc3[nH]ccc3c2Cn2cc(nn2)CO1] |
| 59 | 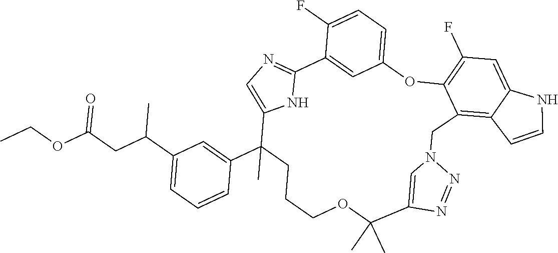 [SMILES: CCOC(=O)CC(C)c1cccc(C2(C)CCCOC(C)(C)c3cn(nn3)Cc3c(c(F)cc4[nH]ccc34)Oc3ccc(F)c(c3)-c3ncc2[nH]3)c1] |
| 60 | 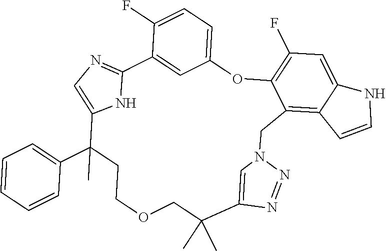 [SMILES: CC1(C)COCCC(C)(c2ccccc2)c2cnc([nH]2)-c2cc(ccc2F)Oc2c(F)cc3[nH]ccc3c2Cn2cc1nn2] |
| 61 | 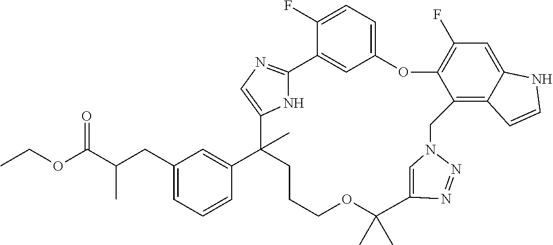 [SMILES: CCOC(=O)C(C)Cc1cccc(C2(C)CCCOC(C)(C)c3cn(nn3)Cc3c(c(F)cc4[nH]ccc34)Oc3ccc(F)c(c3)-c3ncc2[nH]3)c1] |
| 62A | 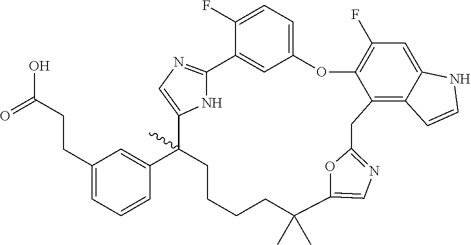 [SMILES: CC1(C)CCCCC(C)(c2cccc(CCC(=O)O)c2)c2cnc([nH]2)-c2cc(ccc2F)Oc2c(F)cc3[nH]ccc3c2CC2=NC=C1O2] |
| 62B | 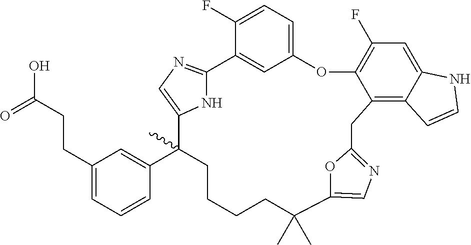 [SMILES: CC1(C)CCCCC(C)(c2cccc(CCC(=O)O)c2)c2cnc([nH]2)-c2cc(ccc2F)Oc2c(F)cc3[nH]ccc3c2CC2=NC=C1O2] |
| 63 | 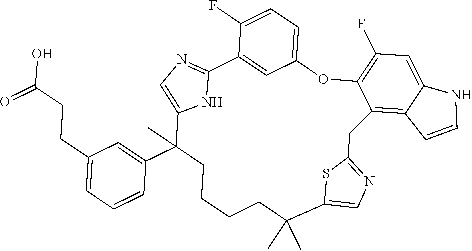 [SMILES: CC1(C)CCCCC(C)(c2cccc(CCC(=O)O)c2)c2cnc([nH]2)-c2cc(ccc2F)Oc2c(F)cc3[nH]ccc3c2CC2=NC=C1S2] |
| 64 | 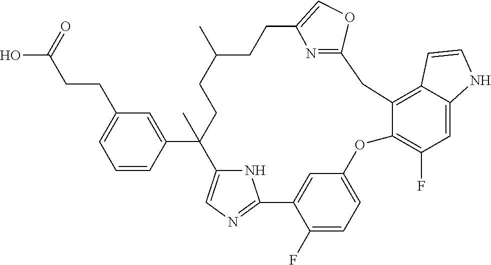 [SMILES: CC1CCc2coc(n2)Cc2c(c(F)cc3[nH]ccc23)Oc2ccc(F)c(c2)-c2ncc([nH]2)C(C)(c2cccc(CCC(=O)O)c2)CC1] |
| 65 | 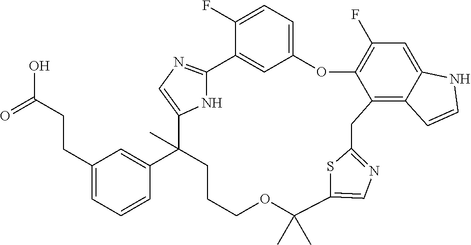 [SMILES: CC1(C)OCCCC(C)(c2cccc(CCC(=O)O)c2)c2cnc([nH]2)-c2cc(ccc2F)Oc2c(F)cc3[nH]ccc3c2CC2=NC=C1S2] |
| 66 | 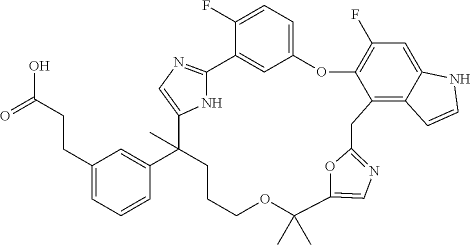 [SMILES: CC1(C)OCCCC(C)(c2cccc(CCC(=O)O)c2)c2cnc([nH]2)-c2cc(ccc2F)Oc2c(F)cc3[nH]ccc3c2CC2=NC=C1O2] |
| 67A | 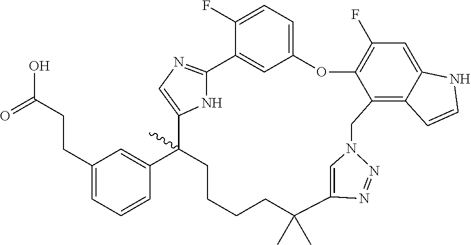 [SMILES: CC1(C)CCCCC(C)(c2cccc(CCC(=O)O)c2)c2cnc([nH]2)-c2cc(ccc2F)Oc2c(F)cc3[nH]ccc3c2Cn2cc1nn2] |
| 67B | 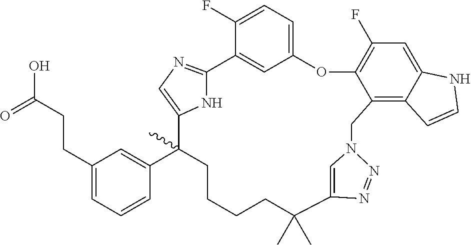 [SMILES: CC1(C)CCCCC(C)(c2cccc(CCC(=O)O)c2)c2cnc([nH]2)-c2cc(ccc2F)Oc2c(F)cc3[nH]ccc3c2Cn2cc1nn2] |
| 68 | 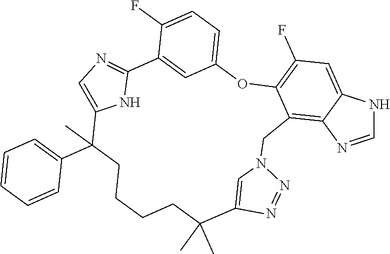 [SMILES: CC1(C)CCCCC(C)(c2ccccc2)c2cnc([nH]2)-c2cc(ccc2F)Oc2c(F)cc3[nH]cnc3c2Cn2cc1nn2] |
| 69 | 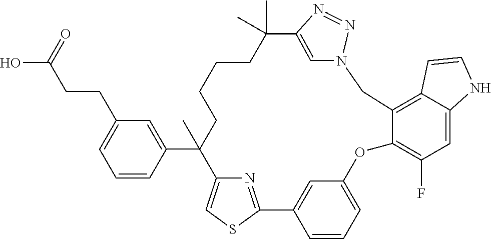 [SMILES: CC1(C)CCCCC(C)(c2cccc(CCC(=O)O)c2)c2csc(n2)-c2cccc(c2)Oc2c(F)cc3[nH]ccc3c2Cn2cc1nn2] |
| 70 | 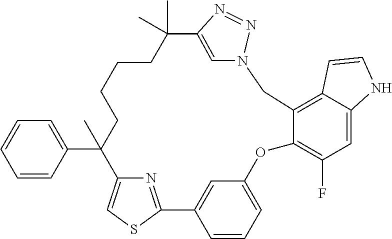 [SMILES: CC1(C)CCCCC(C)(c2ccccc2)c2csc(n2)-c2cccc(c2)Oc2c(F)cc3[nH]ccc3c2Cn2cc1nn2] |
| 71 | 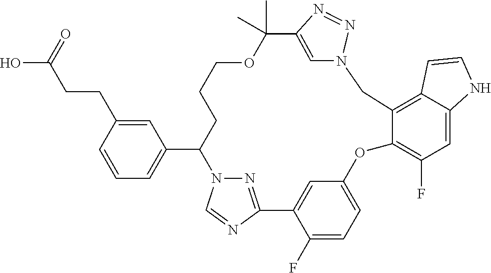 [SMILES: CC1(C)OCCCC(c2cccc(CCC(=O)O)c2)n2cnc(n2)-c2cc(ccc2F)Oc2c(F)cc3[nH]ccc3c2Cn2cc1nn2] |
| 72 | 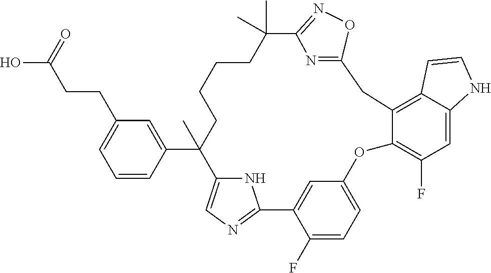 [SMILES: CC1(C)CCCCC(C)(c2cccc(CCC(=O)O)c2)c2cnc([nH]2)-c2cc(ccc2F)Oc2c(F)cc3[nH]ccc3c2Cc2nc1no2] |
| 73A | 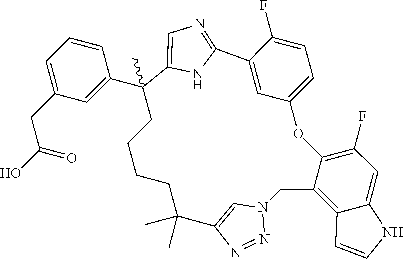 [SMILES: CC1(C)CCCCC(C)(c2cccc(CC(=O)O)c2)c2cnc([nH]2)-c2cc(ccc2F)Oc2c(F)cc3[nH]ccc3c2Cn2cc1nn2] |
| 73B | 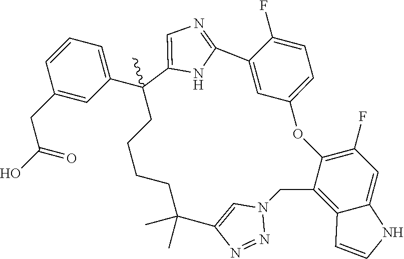 [SMILES: CC1(C)CCCCC(C)(c2cccc(CC(=O)O)c2)c2cnc([nH]2)-c2cc(ccc2F)Oc2c(F)cc3[nH]ccc3c2Cn2cc1nn2] |
| 74 | 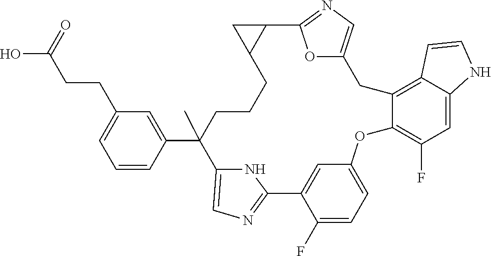 [SMILES: CC1(c2cccc(CCC(=O)O)c2)CCCC2CC2c2ncc(o2)Cc2c(c(F)cc3[nH]ccc23)Oc2ccc(F)c(c2)-c2ncc1[nH]2] |
| 75 | 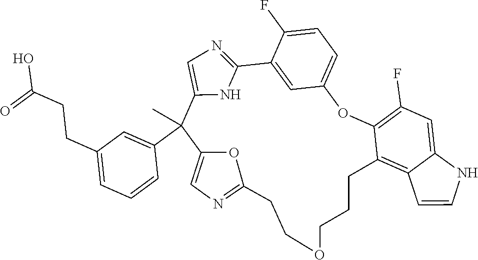 [SMILES: CC1(c2cccc(CCC(=O)O)c2)C2=CN=C(CCOCCCc3c(c(F)cc4[nH]ccc34)Oc3ccc(F)c(c3)-c3ncc1[nH]3)O2] |
| 76 | 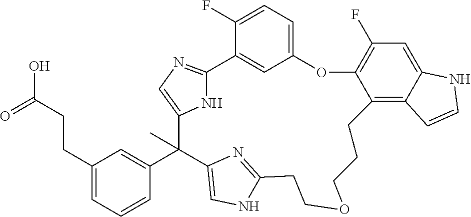 [SMILES: CC1(c2cccc(CCC(=O)O)c2)c2c[nH]c(n2)CCOCCCc2c(c(F)cc3[nH]ccc23)Oc2ccc(F)c(c2)-c2ncc1[nH]2] |
| 77 | 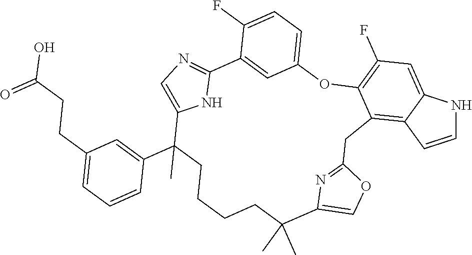 [SMILES: CC1(C)CCCCC(C)(c2cccc(CCC(=O)O)c2)c2cnc([nH]2)-c2cc(ccc2F)Oc2c(F)cc3[nH]ccc3c2Cc2nc1co2] |
| 78A | 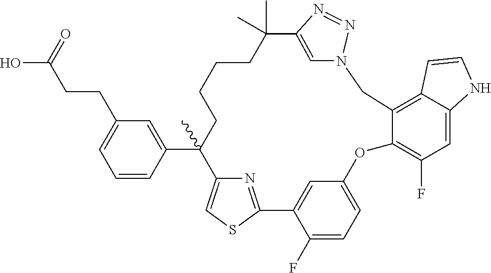 [SMILES: CC1(C)CCCCC(C)(c2cccc(CCC(=O)O)c2)c2csc(n2)-c2cc(ccc2F)Oc2c(F)cc3[nH]ccc3c2Cn2cc1nn2] |
| 78B | 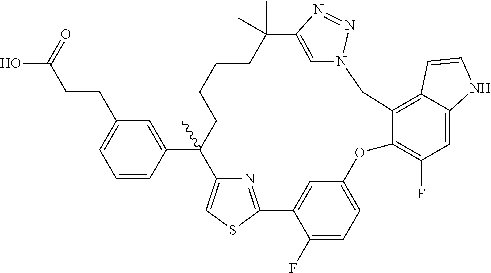 [SMILES: CC1(C)CCCCC(C)(c2cccc(CCC(=O)O)c2)c2csc(n2)-c2cc(ccc2F)Oc2c(F)cc3[nH]ccc3c2Cn2cc1nn2] |
| 79 | 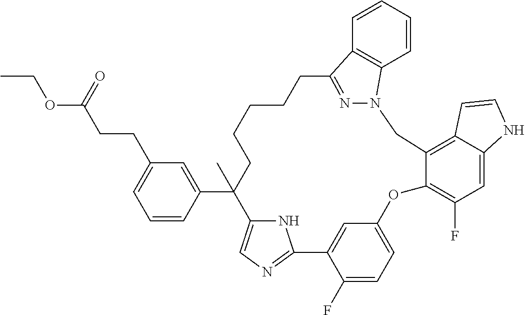 [SMILES: CCOC(=O)CCc1cccc(C2(C)CCCCCc3nn(c4ccccc34)Cc3c(c(F)cc4[nH]ccc34)Oc3ccc(F)c(c3)-c3ncc2[nH]3)c1] |
| 80 | 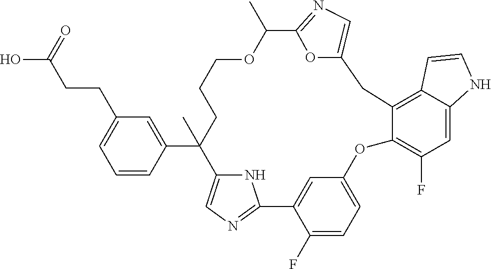 [SMILES: CC1OCCCC(C)(c2cccc(CCC(=O)O)c2)c2cnc([nH]2)-c2cc(ccc2F)Oc2c(F)cc3[nH]ccc3c2CC2=CN=C1O2] |
| 81 | 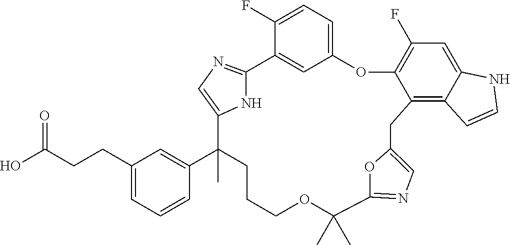 [SMILES: CC1(C)OCCCC(C)(c2cccc(CCC(=O)O)c2)c2cnc([nH]2)-c2cc(ccc2F)Oc2c(F)cc3[nH]ccc3c2CC2=CN=C1O2] |
| 82 | 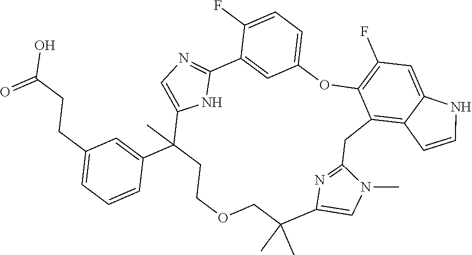 [SMILES: Cn1cc2nc1Cc1c(c(F)cc3[nH]ccc13)Oc1ccc(F)c(c1)-c1ncc([nH]1)C(C)(c1cccc(CCC(=O)O)c1)CCOCC2(C)C] |
| 83A | 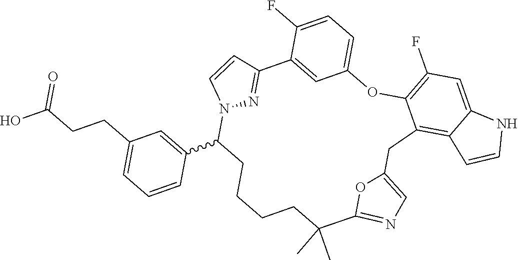 |
| 83B | 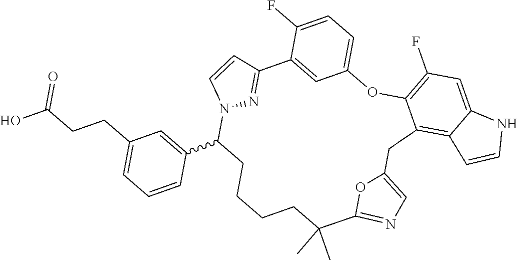 |
| 84 | 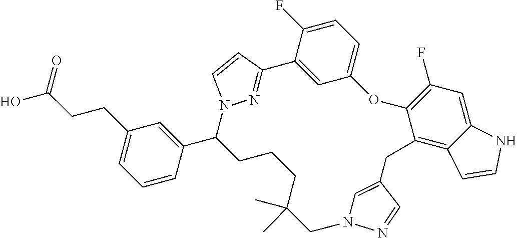 [SMILES: CC1(C)CCCC(c2cccc(CCC(=O)O)c2)n2ccc(n2)-c2cc(ccc2F)Oc2c(F)cc3[nH]ccc3c2Cc2cnn(c2)C1] |
| 85 | 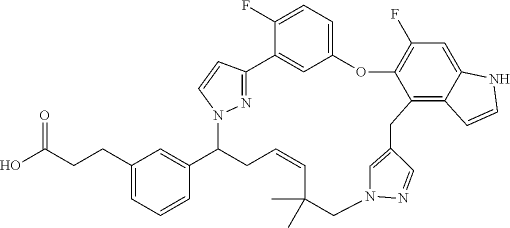 [SMILES: CC1(C)/C=C\CC(c2cccc(CCC(=O)O)c2)n2ccc(n2)-c2cc(ccc2F)Oc2c(F)cc3[nH]ccc3c2Cc2cnn(c2)C1] |
| 86 | 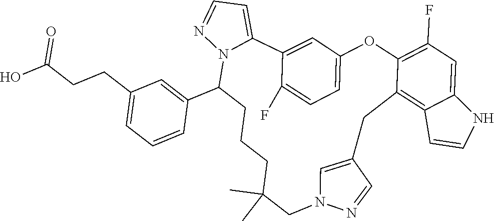 [SMILES: CC1(C)CCCC(c2cccc(CCC(=O)O)c2)n2nccc2-c2cc(ccc2F)Oc2c(F)cc3[nH]ccc3c2Cc2cnn(c2)C1] |
| 87 | 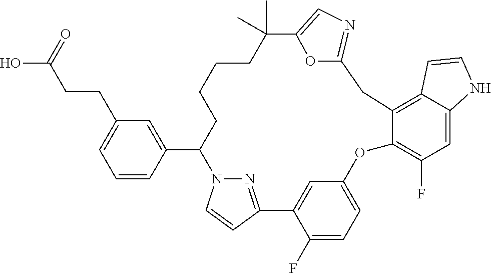 [SMILES: CC1(C)CCCCC(c2cccc(CCC(=O)O)c2)n2ccc(n2)-c2cc(ccc2F)Oc2c(F)cc3[nH]ccc3c2CC2=NC=C1O2] |
| 88A | 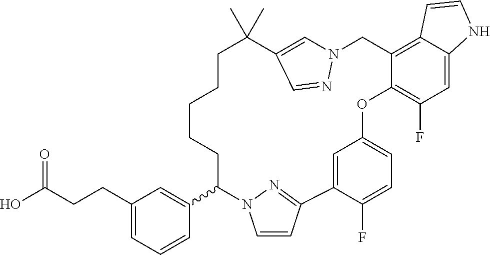 [SMILES: CC1(C)CCCCCC(c2cccc(CCC(=O)O)c2)n2ccc(n2)-c2cc(ccc2F)Oc2c(F)cc3[nH]ccc3c2Cn2cc1cn2] |
| 88B | 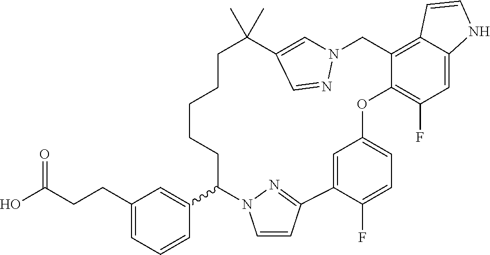 [SMILES: CC1(C)CCCCCC(c2cccc(CCC(=O)O)c2)n2ccc(n2)-c2cc(ccc2F)Oc2c(F)cc3[nH]ccc3c2Cn2cc1cn2] |
| 89A | 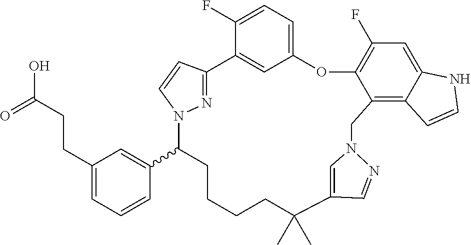 [SMILES: CC1(C)CCCCC(c2cccc(CCC(=O)O)c2)n2ccc(n2)-c2cc(ccc2F)Oc2c(F)cc3[nH]ccc3c2Cn2cc1cn2] |
| 89B | 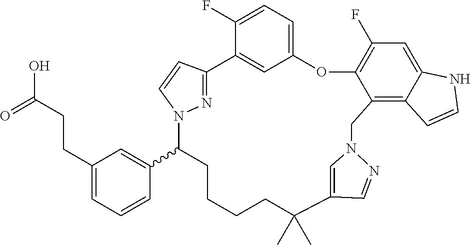 [SMILES: CC1(C)CCCCC(c2cccc(CCC(=O)O)c2)n2ccc(n2)-c2cc(ccc2F)Oc2c(F)cc3[nH]ccc3c2Cn2cc1cn2] |
| 89C | 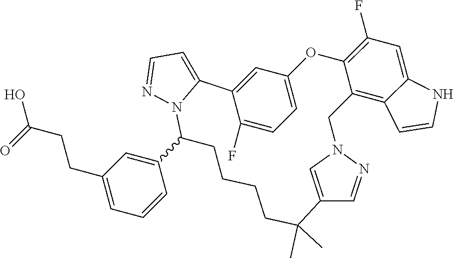 [SMILES: CC1(C)CCCCC(c2cccc(CCC(=O)O)c2)n2nccc2-c2cc(ccc2F)Oc2c(F)cc3[nH]ccc3c2Cn2cc1cn2] |
| 89D | 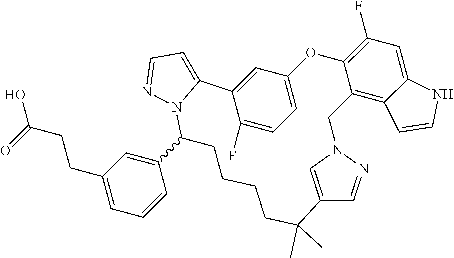 [SMILES: CC1(C)CCCCC(c2cccc(CCC(=O)O)c2)n2nccc2-c2cc(ccc2F)Oc2c(F)cc3[nH]ccc3c2Cn2cc1cn2] |
| 90A | 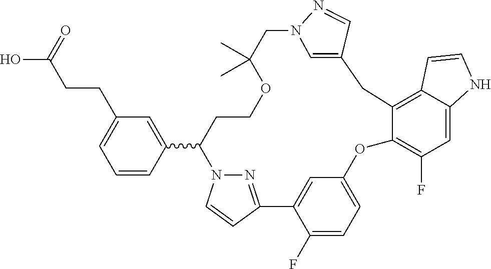 [SMILES: CC1(C)Cn2cc(cn2)Cc2c(c(F)cc3[nH]ccc23)Oc2ccc(F)c(c2)-c2ccn(n2)C(c2cccc(CCC(=O)O)c2)CCO1] |
| 90B | 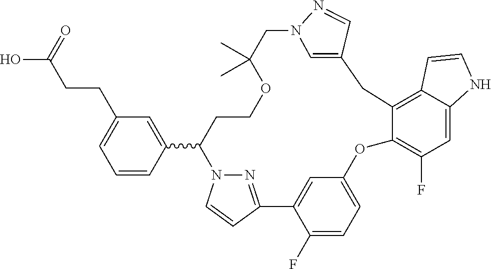 [SMILES: CC1(C)Cn2cc(cn2)Cc2c(c(F)cc3[nH]ccc23)Oc2ccc(F)c(c2)-c2ccn(n2)C(c2cccc(CCC(=O)O)c2)CCO1] |
| 91 | 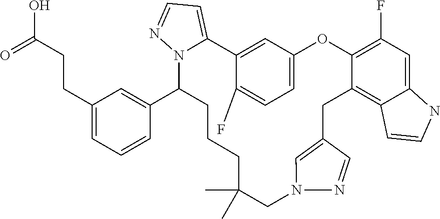 [SMILES: CC1(C)CCCC(c2cccc(CCC(=O)O)c2)n2nccc2-c2cc(ccc2F)Oc2c(F)cc3[nH]ccc3c2Cc2cnn(c2)C1] |
| 92A | 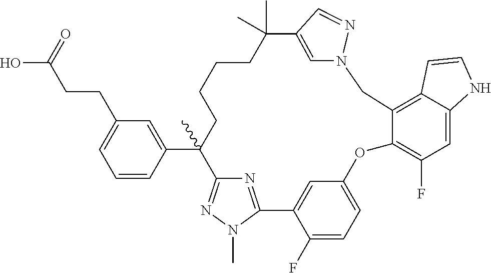 [SMILES: Cn1nc2nc1-c1cc(ccc1F)Oc1c(F)cc3[nH]ccc3c1Cn1cc(cn1)C(C)(C)CCCCC2(C)c1cccc(CCC(=O)O)c1] |
| 92B | 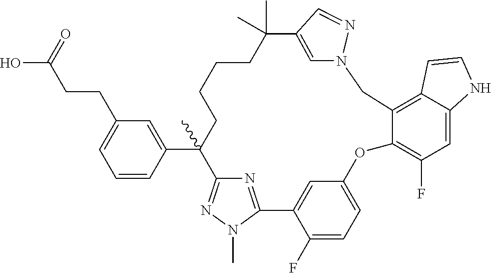 [SMILES: Cn1nc2nc1-c1cc(ccc1F)Oc1c(F)cc3[nH]ccc3c1Cn1cc(cn1)C(C)(C)CCCCC2(C)c1cccc(CCC(=O)O)c1] |
| 93A | 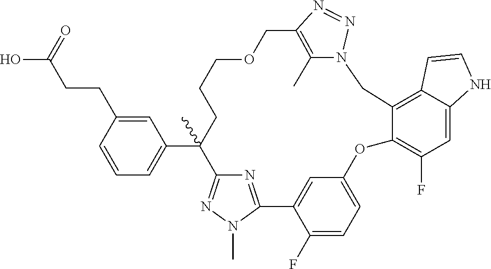 [SMILES: Cc1c2nnn1Cc1c(c(F)cc3[nH]ccc13)Oc1ccc(F)c(c1)-c1nc(nn1C)C(C)(c1cccc(CCC(=O)O)c1)CCCOC2] |
| 93B | 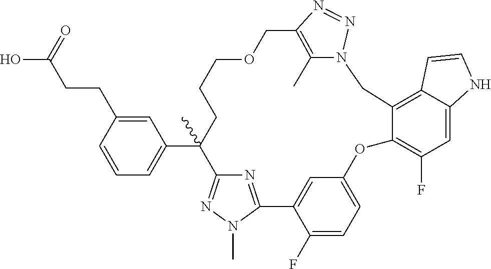 [SMILES: Cc1c2nnn1Cc1c(c(F)cc3[nH]ccc13)Oc1ccc(F)c(c1)-c1nc(nn1C)C(C)(c1cccc(CCC(=O)O)c1)CCCOC2] |
| 94A | 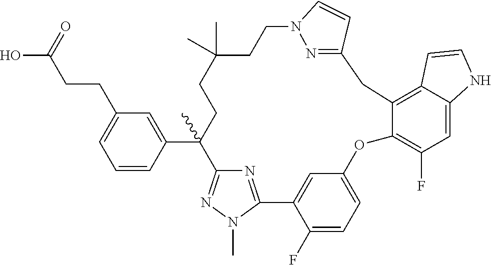 [SMILES: Cn1nc2nc1-c1cc(ccc1F)Oc1c(F)cc3[nH]ccc3c1Cc1ccn(n1)CCC(C)(C)CCC2(C)c1cccc(CCC(=O)O)c1] |
| 94B | 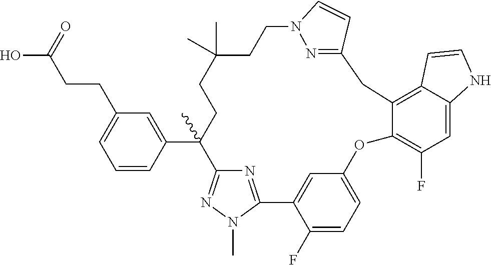 [SMILES: Cn1nc2nc1-c1cc(ccc1F)Oc1c(F)cc3[nH]ccc3c1Cc1ccn(n1)CCC(C)(C)CCC2(C)c1cccc(CCC(=O)O)c1] |
| 95 | 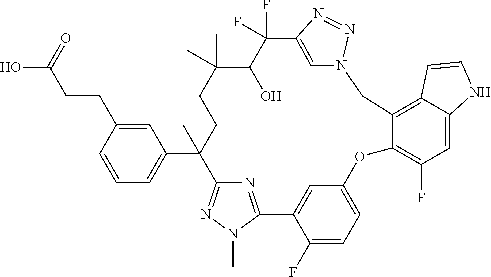 [SMILES: Cn1nc2nc1-c1cc(ccc1F)Oc1c(F)cc3[nH]ccc3c1Cn1cc(nn1)C(F)(F)C(O)C(C)(C)CCC2(C)c1cccc(CCC(=O)O)c1] |
| 96A | 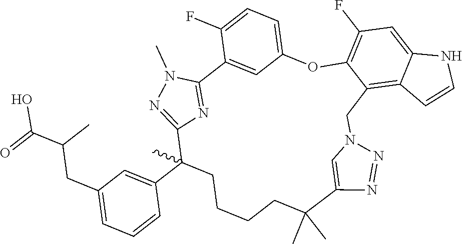 [SMILES: CC(Cc1cccc(C2(C)CCCCC(C)(C)c3cn(nn3)Cc3c(c(F)cc4[nH]ccc34)Oc3ccc(F)c(c3)-c3nc2nn3C)c1)C(=O)O] |
| 96B | 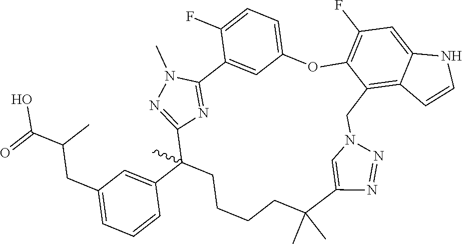 [SMILES: CC(Cc1cccc(C2(C)CCCCC(C)(C)c3cn(nn3)Cc3c(c(F)cc4[nH]ccc34)Oc3ccc(F)c(c3)-c3nc2nn3C)c1)C(=O)O] |
| 97A | 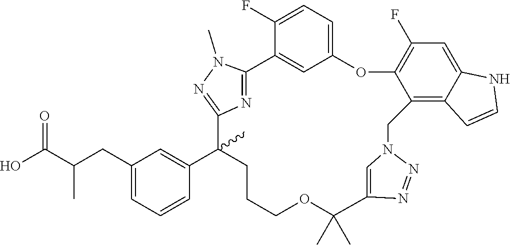 [SMILES: CC(Cc1cccc(C2(C)CCCOC(C)(C)c3cn(nn3)Cc3c(c(F)cc4[nH]ccc34)Oc3ccc(F)c(c3)-c3nc2nn3C)c1)C(=O)O] |
| 97B | 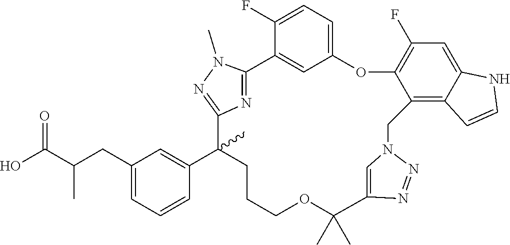 [SMILES: CC(Cc1cccc(C2(C)CCCOC(C)(C)c3cn(nn3)Cc3c(c(F)cc4[nH]ccc34)Oc3ccc(F)c(c3)-c3nc2nn3C)c1)C(=O)O] |
| 98A | 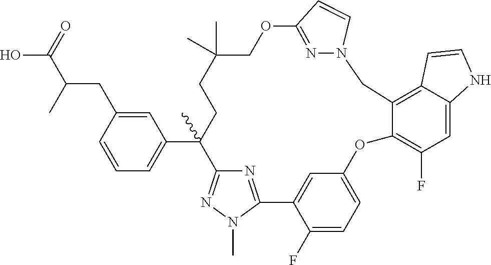 [SMILES: CC(Cc1cccc(C2(C)CCC(C)(C)COc3ccn(n3)Cc3c(c(F)cc4[nH]ccc34)Oc3ccc(F)c(c3)-c3nc2nn3C)c1)C(=O)O] |
| 98B | 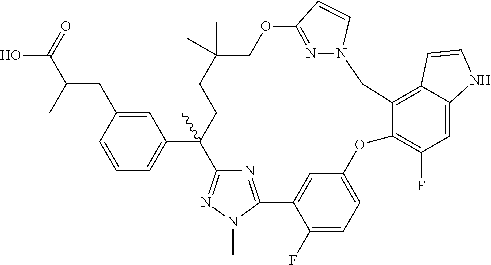 [SMILES: CC(Cc1cccc(C2(C)CCC(C)(C)COc3ccn(n3)Cc3c(c(F)cc4[nH]ccc34)Oc3ccc(F)c(c3)-c3nc2nn3C)c1)C(=O)O] |
| 99A | 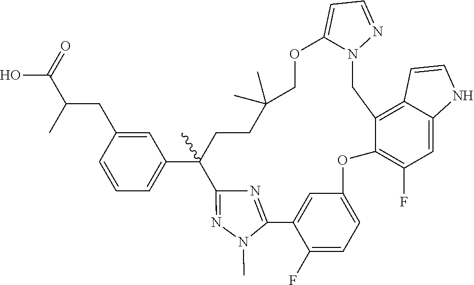 [SMILES: CC(Cc1cccc(C2(C)CCC(C)(C)COc3ccnn3Cc3c(c(F)cc4[nH]ccc34)Oc3ccc(F)c(c3)-c3nc2nn3C)c1)C(=O)O] |
| 99B | 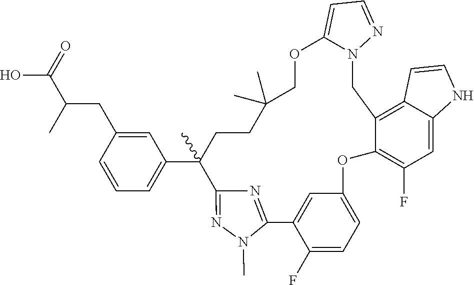 [SMILES: CC(Cc1cccc(C2(C)CCC(C)(C)COc3ccnn3Cc3c(c(F)cc4[nH]ccc34)Oc3ccc(F)c(c3)-c3nc2nn3C)c1)C(=O)O] |
| 100A | 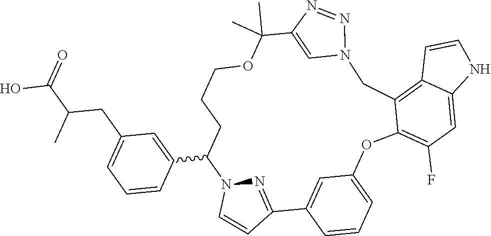 |
| 100B | 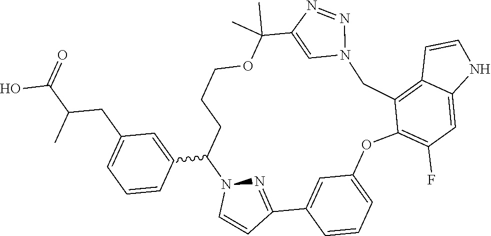 |
| 101A | 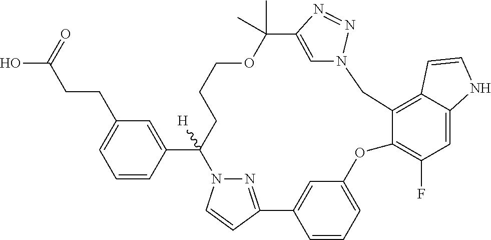 [SMILES: CC1(C)OCCCC(c2cccc(CCC(=O)O)c2)n2ccc(n2)-c2cccc(c2)Oc2c(F)cc3[nH]ccc3c2Cn2cc1nn2] |
| 101B | 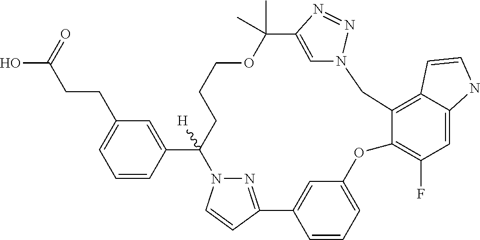 [SMILES: CC1(C)OCCCC(c2cccc(CCC(=O)O)c2)n2ccc(n2)-c2cccc(c2)Oc2c(F)cc3[nH]ccc3c2Cn2cc1nn2] |
| 102 | 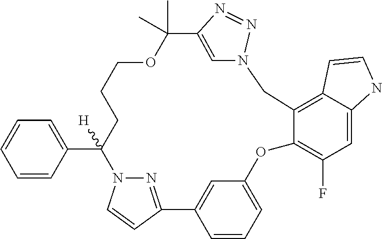 [SMILES: CC1(C)OCCCC(c2ccccc2)n2ccc(n2)-c2cccc(c2)Oc2c(F)cc3[nH]ccc3c2Cn2cc1nn2] |
| 103A | 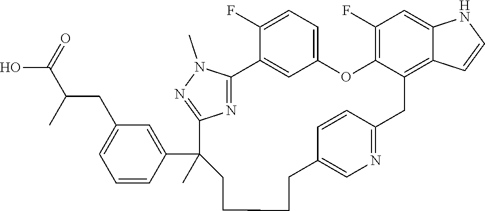 [SMILES: CC(Cc1cccc(C2(C)CCCCc3ccc(nc3)Cc3c(c(F)cc4[nH]ccc34)Oc3ccc(F)c(c3)-c3nc2nn3C)c1)C(=O)O] |
| 103B | 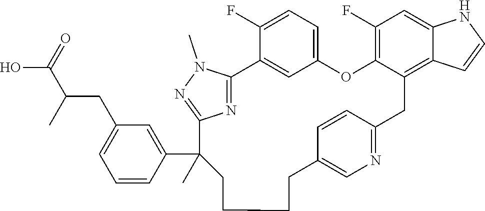 [SMILES: CC(Cc1cccc(C2(C)CCCCc3ccc(nc3)Cc3c(c(F)cc4[nH]ccc34)Oc3ccc(F)c(c3)-c3nc2nn3C)c1)C(=O)O] |
| 104A | 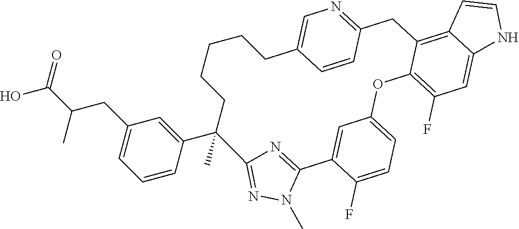 [SMILES: CC(Cc1cccc([C@@]2(C)CCCCCc3ccc(nc3)Cc3c(c(F)cc4[nH]ccc34)Oc3ccc(F)c(c3)-c3nc2nn3C)c1)C(=O)O] |
| 104B | 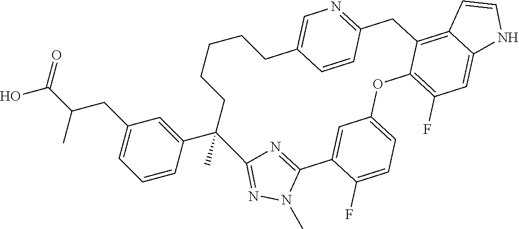 [SMILES: CC(Cc1cccc([C@@]2(C)CCCCCc3ccc(nc3)Cc3c(c(F)cc4[nH]ccc34)Oc3ccc(F)c(c3)-c3nc2nn3C)c1)C(=O)O] |
| 105A | 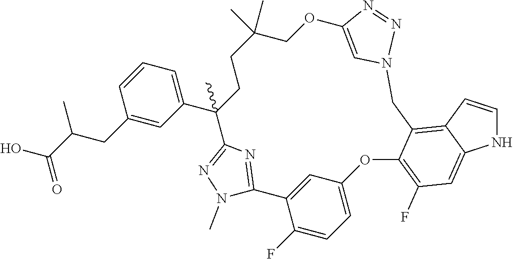 [SMILES: CC(Cc1cccc(C2(C)CCC(C)(C)COc3cn(nn3)Cc3c(c(F)cc4[nH]ccc34)Oc3ccc(F)c(c3)-c3nc2nn3C)c1)C(=O)O] |
| 105B | 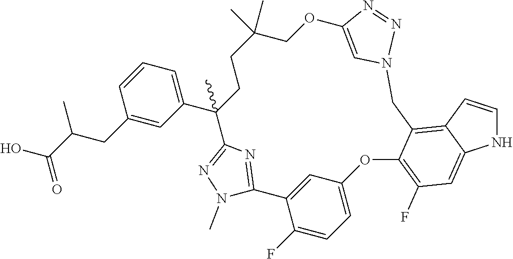 [SMILES: CC(Cc1cccc(C2(C)CCC(C)(C)COc3cn(nn3)Cc3c(c(F)cc4[nH]ccc34)Oc3ccc(F)c(c3)-c3nc2nn3C)c1)C(=O)O] |
| 106 | 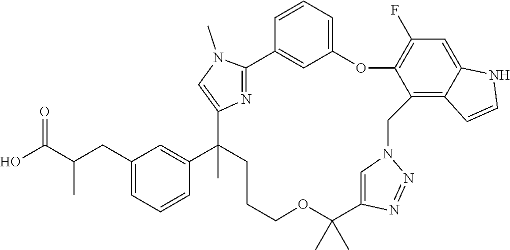 [SMILES: CC(Cc1cccc(C2(C)CCCOC(C)(C)c3cn(nn3)Cc3c(c(F)cc4[nH]ccc34)Oc3cccc(c3)-c3nc2cn3C)c1)C(=O)O] |
| 107 | 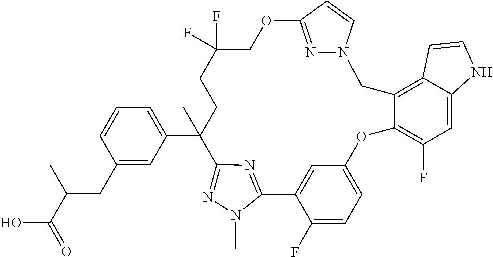 [SMILES: CC(Cc1cccc(C2(C)CCC(F)(F)COc3ccn(n3)Cc3c(c(F)cc4[nH]ccc34)Oc3ccc(F)c(c3)-c3nc2nn3C)c1)C(=O)O] |
| 108 | 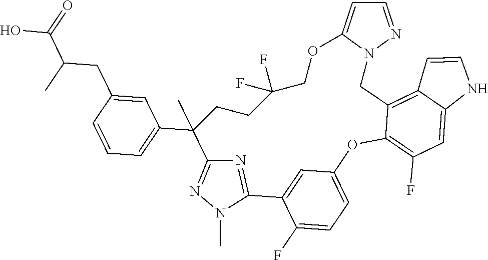 [SMILES: CC(Cc1cccc(C2(C)CCC(F)(F)COc3ccnn3Cc3c(c(F)cc4[nH]ccc34)Oc3ccc(F)c(c3)-c3nc2nn3C)c1)C(=O)O] |
| 109A | 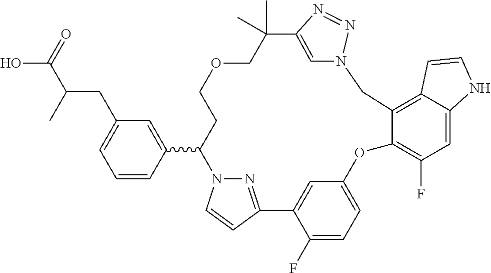 [SMILES: CC(Cc1cccc(C2CCOCC(C)(C)c3cn(nn3)Cc3c(c(F)cc4[nH]ccc34)Oc3ccc(F)c(c3)-c3ccn2n3)c1)C(=O)O] |
| 109B | 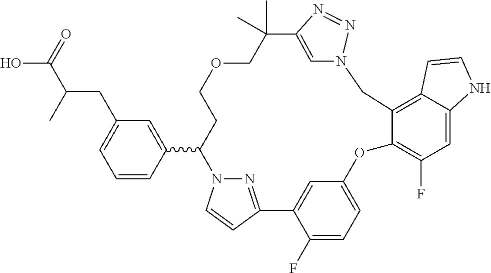 [SMILES: CC(Cc1cccc(C2CCOCC(C)(C)c3cn(nn3)Cc3c(c(F)cc4[nH]ccc34)Oc3ccc(F)c(c3)-c3ccn2n3)c1)C(=O)O] |
| 110 | 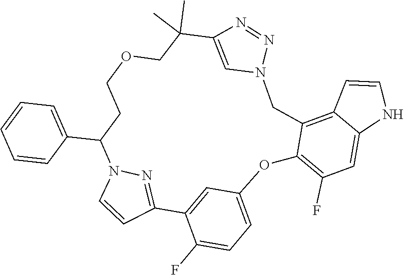 [SMILES: CC1(C)COCCC(c2ccccc2)n2ccc(n2)-c2cc(ccc2F)Oc2c(F)cc3[nH]ccc3c2Cn2cc1nn2] |
| 111A | 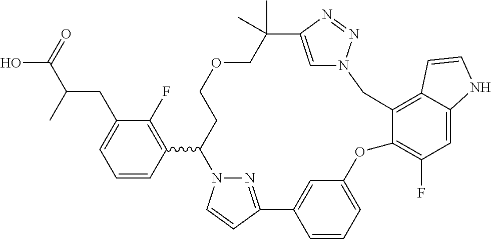 [SMILES: CC(Cc1cccc(C2CCOCC(C)(C)c3cn(nn3)Cc3c(c(F)cc4[nH]ccc34)Oc3cccc(c3)-c3ccn2n3)c1F)C(=O)O] |
| 111B | 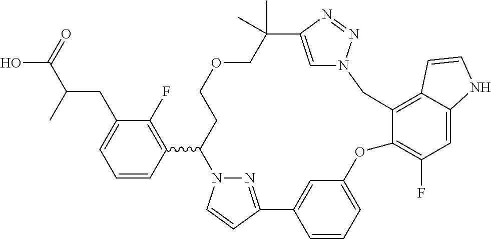 [SMILES: CC(Cc1cccc(C2CCOCC(C)(C)c3cn(nn3)Cc3c(c(F)cc4[nH]ccc34)Oc3cccc(c3)-c3ccn2n3)c1F)C(=O)O] |
| 112A | 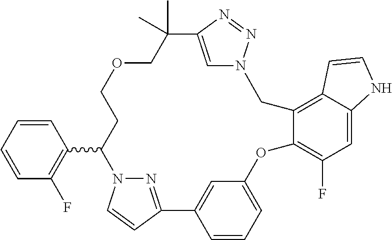 [SMILES: CC1(C)COCCC(c2ccccc2F)n2ccc(n2)-c2cccc(c2)Oc2c(F)cc3[nH]ccc3c2Cn2cc1nn2] |
| 112B | 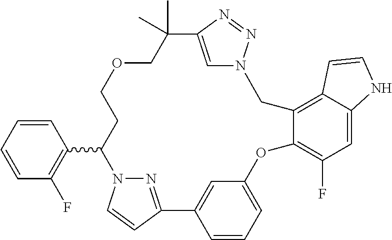 [SMILES: CC1(C)COCCC(c2ccccc2F)n2ccc(n2)-c2cccc(c2)Oc2c(F)cc3[nH]ccc3c2Cn2cc1nn2] |
| 113A | 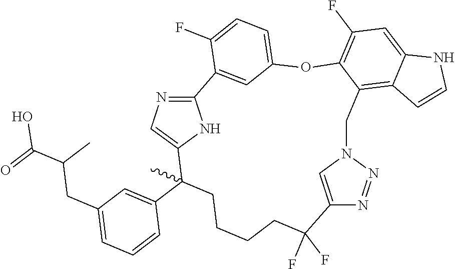 [SMILES: CC(Cc1cccc(C2(C)CCCCC(F)(F)c3cn(nn3)Cc3c(c(F)cc4[nH]ccc34)Oc3ccc(F)c(c3)-c3ncc2[nH]3)c1)C(=O)O] |
| 113B | 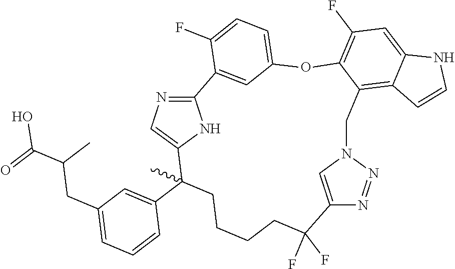 [SMILES: CC(Cc1cccc(C2(C)CCCCC(F)(F)c3cn(nn3)Cc3c(c(F)cc4[nH]ccc34)Oc3ccc(F)c(c3)-c3ncc2[nH]3)c1)C(=O)O] |
| 114A | 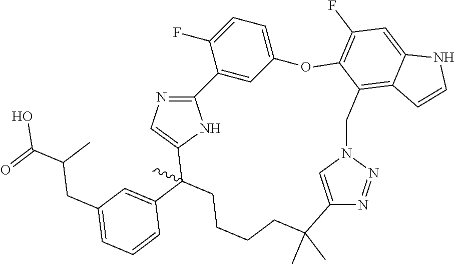 [SMILES: CC(Cc1cccc(C2(C)CCCCC(C)(C)c3cn(nn3)Cc3c(c(F)cc4[nH]ccc34)Oc3ccc(F)c(c3)-c3ncc2[nH]3)c1)C(=O)O] |
| 114B | 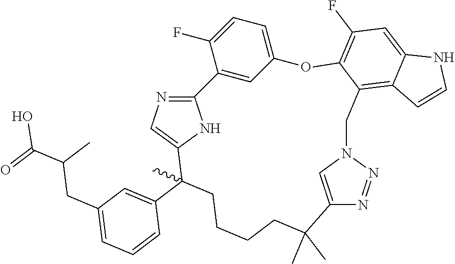 [SMILES: CC(Cc1cccc(C2(C)CCCCC(C)(C)c3cn(nn3)Cc3c(c(F)cc4[nH]ccc34)Oc3ccc(F)c(c3)-c3ncc2[nH]3)c1)C(=O)O] |
| 115A | 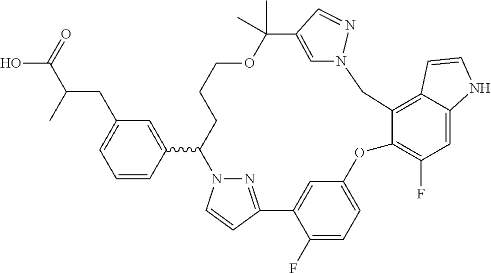 [SMILES: CC(Cc1cccc(C2CCCOC(C)(C)c3cnn(c3)Cc3c(c(F)cc4[nH]ccc34)Oc3ccc(F)c(c3)-c3ccn2n3)c1)C(=O)O] |
| 115B | 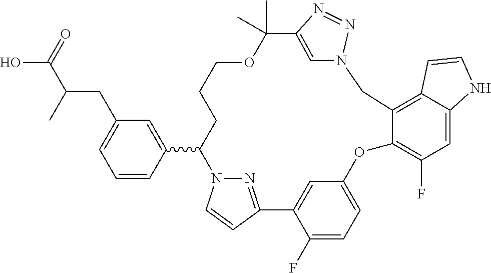 [SMILES: CC(Cc1cccc(C2CCCOC(C)(C)c3cn(nn3)Cc3c(c(F)cc4[nH]ccc34)Oc3ccc(F)c(c3)-c3ccn2n3)c1)C(=O)O] |
| 116A | 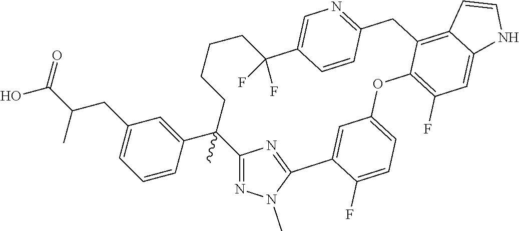 [SMILES: CC(Cc1cccc(C2(C)CCCCC(F)(F)c3ccc(nc3)Cc3c(c(F)cc4[nH]ccc34)Oc3ccc(F)c(c3)-c3nc2nn3C)c1)C(=O)O] |
| 116B | 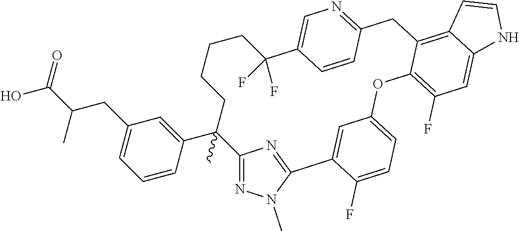 [SMILES: CC(Cc1cccc(C2(C)CCCCC(F)(F)c3ccc(nc3)Cc3c(c(F)cc4[nH]ccc34)Oc3ccc(F)c(c3)-c3nc2nn3C)c1)C(=O)O] |
| 117A | 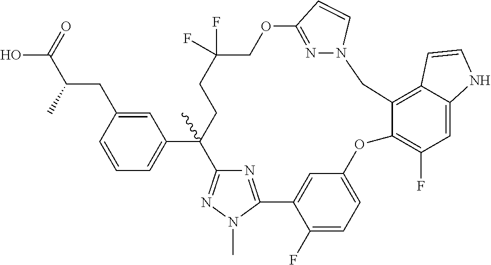 [SMILES: C[C@@H](Cc1cccc(C2(C)CCC(F)(F)COc3ccn(n3)Cc3c(c(F)cc4[nH]ccc34)Oc3ccc(F)c(c3)-c3nc2nn3C)c1)C(=O)O] |
| 117B | 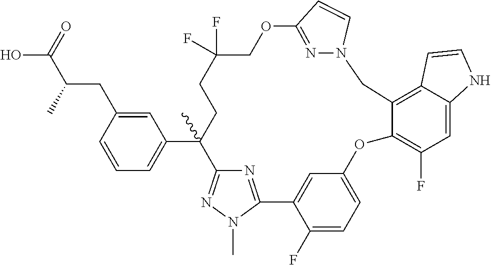 [SMILES: C[C@@H](Cc1cccc(C2(C)CCC(F)(F)COc3ccn(n3)Cc3c(c(F)cc4[nH]ccc34)Oc3ccc(F)c(c3)-c3nc2nn3C)c1)C(=O)O] |
| 118A | 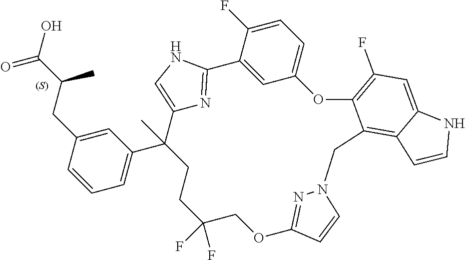 |
| 118B | 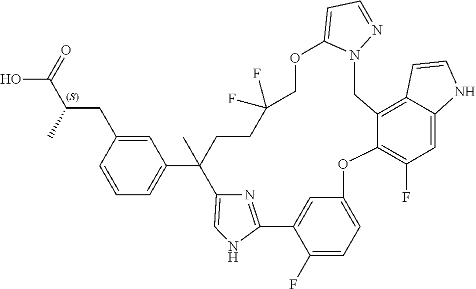 |
| 119 | 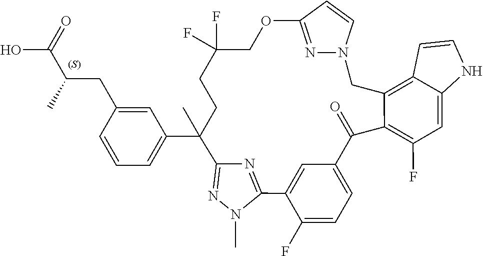 |
| 120A | 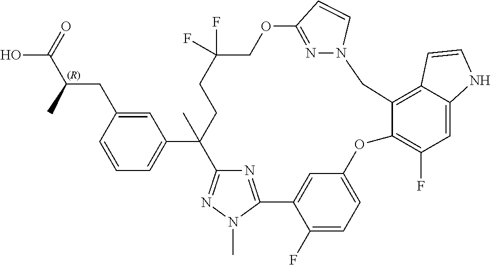 |
| 120B | 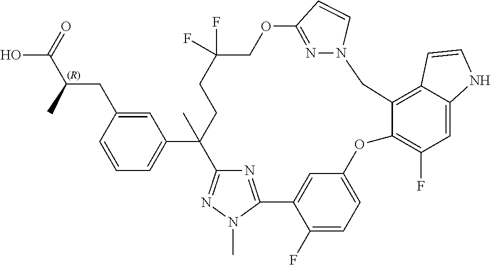 |
| 121A | 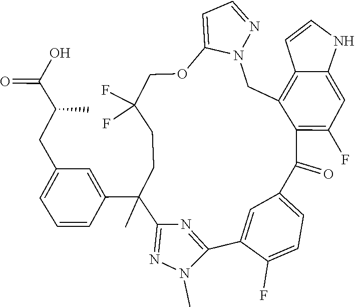 [SMILES: C[C@H](Cc1cccc(C2(C)CCC(F)(F)COc3ccnn3Cc3c(c(F)cc4[nH]ccc34)C(=O)c3ccc(F)c(c3)-c3nc2nn3C)c1)C(=O)O] |
| 121B | 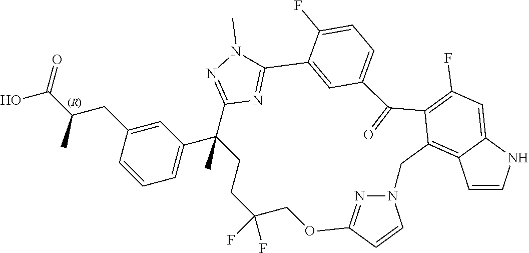 |
| 121C | 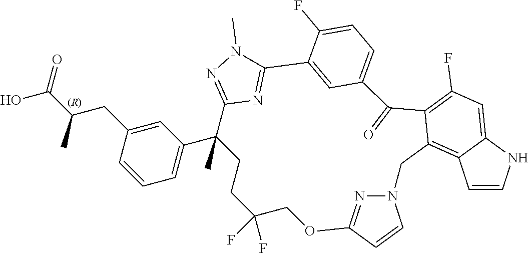 |
| 122 | 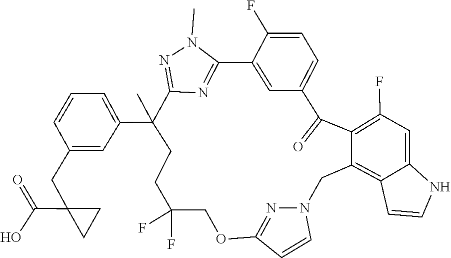 [SMILES: Cn1nc2nc1-c1cc(ccc1F)C(=O)c1c(F)cc3[nH]ccc3c1Cn1ccc(n1)OCC(F)(F)CCC2(C)c1cccc(CC2(C(=O)O)CC2)c1] |
| 123 | 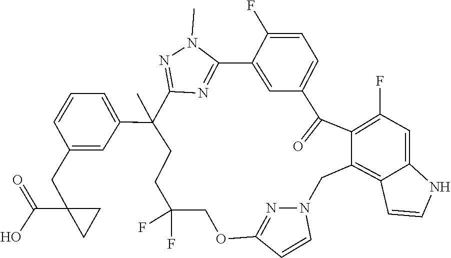 [SMILES: Cn1nc2nc1-c1cc(ccc1F)C(=O)c1c(F)cc3[nH]ccc3c1Cn1ccc(n1)OCC(F)(F)CCC2(C)c1cccc(CC2(C(=O)O)CC2)c1] |
| 124A | 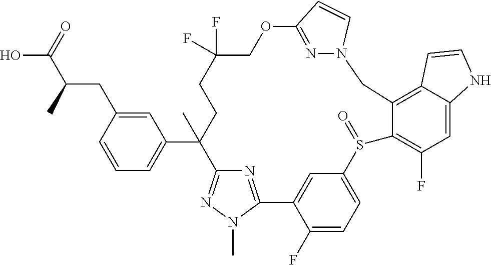 [SMILES: C[C@H](Cc1cccc(C2(C)CCC(F)(F)COc3ccn(n3)Cc3c(c(F)cc4[nH]ccc34)S(=O)c3ccc(F)c(c3)-c3nc2nn3C)c1)C(=O)O] |
| 124B | 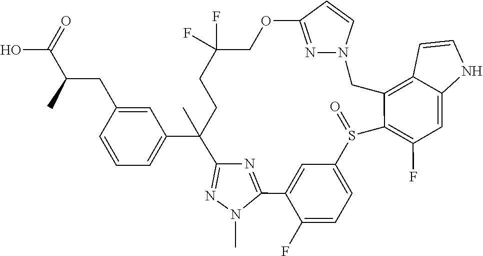 [SMILES: C[C@H](Cc1cccc(C2(C)CCC(F)(F)COc3ccn(n3)Cc3c(c(F)cc4[nH]ccc34)S(=O)c3ccc(F)c(c3)-c3nc2nn3C)c1)C(=O)O] |
| 125A | 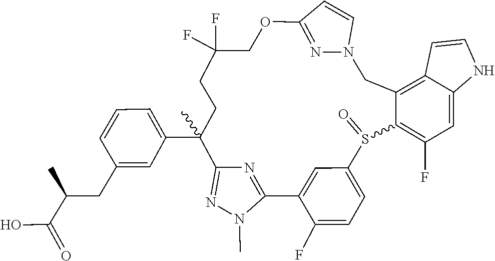 [SMILES: C[C@@H](Cc1cccc(C2(C)CCC(F)(F)COc3ccn(n3)Cc3c(c(F)cc4[nH]ccc34)S(=O)c3ccc(F)c(c3)-c3nc2nn3C)c1)C(=O)O] |
| 125B | 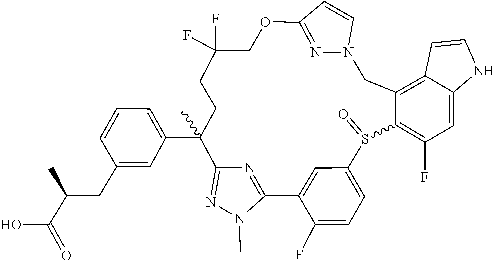 [SMILES: C[C@@H](Cc1cccc(C2(C)CCC(F)(F)COc3ccn(n3)Cc3c(c(F)cc4[nH]ccc34)S(=O)c3ccc(F)c(c3)-c3nc2nn3C)c1)C(=O)O] |
| 125C | 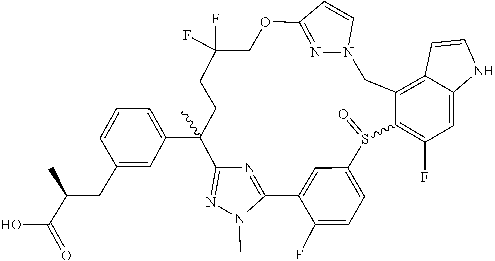 [SMILES: C[C@@H](Cc1cccc(C2(C)CCC(F)(F)COc3ccn(n3)Cc3c(c(F)cc4[nH]ccc34)S(=O)c3ccc(F)c(c3)-c3nc2nn3C)c1)C(=O)O] |
| 125D | 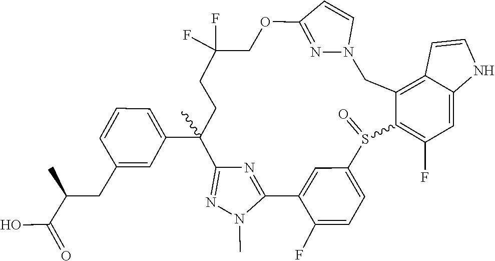 [SMILES: C[C@@H](Cc1cccc(C2(C)CCC(F)(F)COc3ccn(n3)Cc3c(c(F)cc4[nH]ccc34)S(=O)c3ccc(F)c(c3)-c3nc2nn3C)c1)C(=O)O] |
| 126A | 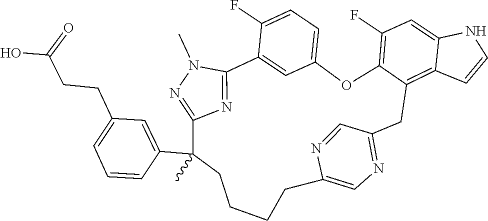 [SMILES: Cn1nc2nc1-c1cc(ccc1F)Oc1c(F)cc3[nH]ccc3c1Cc1cnc(cn1)CCCCC2(C)c1cccc(CCC(=O)O)c1] |
| 126B | 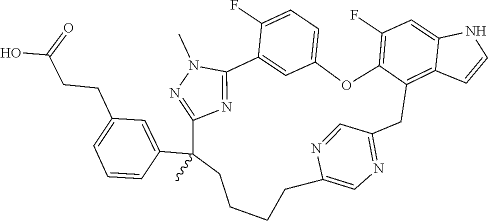 [SMILES: Cn1nc2nc1-c1cc(ccc1F)Oc1c(F)cc3[nH]ccc3c1Cc1cnc(cn1)CCCCC2(C)c1cccc(CCC(=O)O)c1] |
| 127A | 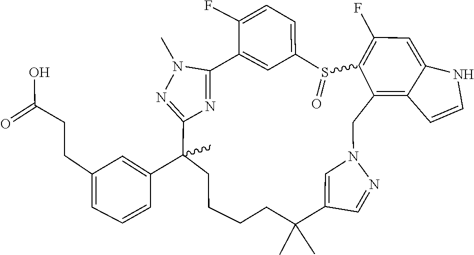 [SMILES: Cn1nc2nc1-c1cc(ccc1F)S(=O)c1c(F)cc3[nH]ccc3c1Cn1cc(cn1)C(C)(C)CCCCC2(C)c1cccc(CCC(=O)O)c1] |
| 127B | 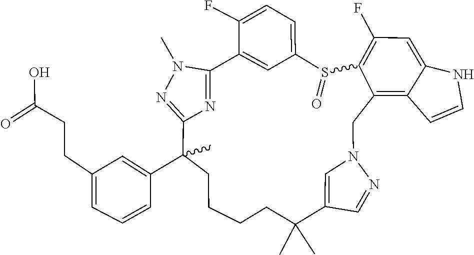 [SMILES: Cn1nc2nc1-c1cc(ccc1F)S(=O)c1c(F)cc3[nH]ccc3c1Cn1cc(cn1)C(C)(C)CCCCC2(C)c1cccc(CCC(=O)O)c1] |
| 127C | 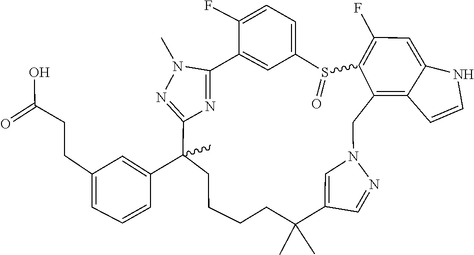 [SMILES: Cn1nc2nc1-c1cc(ccc1F)S(=O)c1c(F)cc3[nH]ccc3c1Cn1cc(cn1)C(C)(C)CCCCC2(C)c1cccc(CCC(=O)O)c1] |
| 127D | 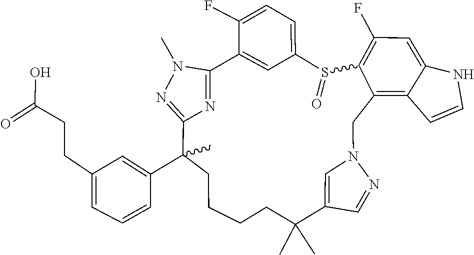 [SMILES: Cn1nc2nc1-c1cc(ccc1F)S(=O)c1c(F)cc3[nH]ccc3c1Cn1cc(cn1)C(C)(C)CCCCC2(C)c1cccc(CCC(=O)O)c1] |
| 128 | 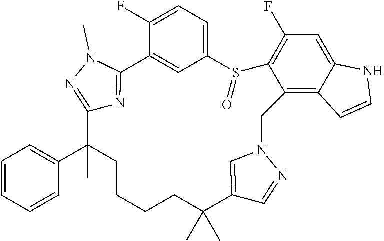 [SMILES: Cn1nc2nc1-c1cc(ccc1F)S(=O)c1c(F)cc3[nH]ccc3c1Cn1cc(cn1)C(C)(C)CCCCC2(C)c1ccccc1] |
| 129A | 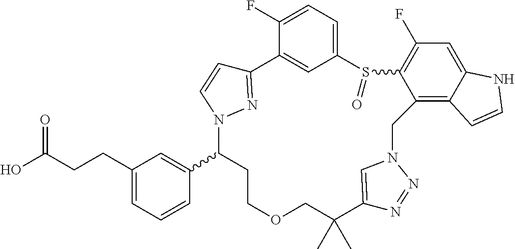 [SMILES: CC1(C)COCCC(c2cccc(CCC(=O)O)c2)n2ccc(n2)-c2cc(ccc2F)S(=O)c2c(F)cc3[nH]ccc3c2Cn2cc1nn2] |
| 129B | 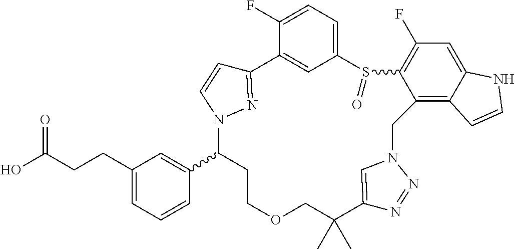 [SMILES: CC1(C)COCCC(c2cccc(CCC(=O)O)c2)n2ccc(n2)-c2cc(ccc2F)S(=O)c2c(F)cc3[nH]ccc3c2Cn2cc1nn2] |
| 130A | 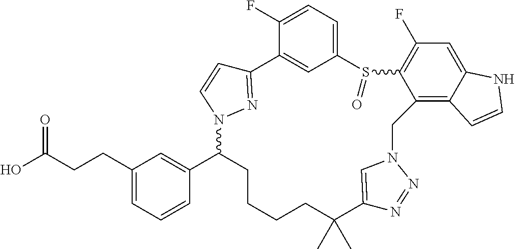 [SMILES: CC1(C)CCCCC(c2cccc(CCC(=O)O)c2)n2ccc(n2)-c2cc(ccc2F)S(=O)c2c(F)cc3[nH]ccc3c2Cn2cc1nn2] |
| 130B | 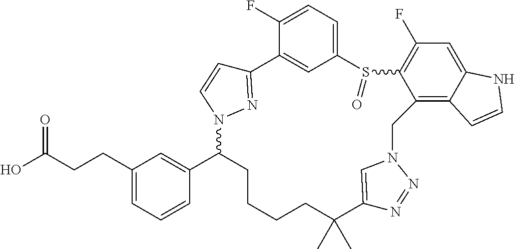 [SMILES: CC1(C)CCCCC(c2cccc(CCC(=O)O)c2)n2ccc(n2)-c2cc(ccc2F)S(=O)c2c(F)cc3[nH]ccc3c2Cn2cc1nn2] |
| 131A | 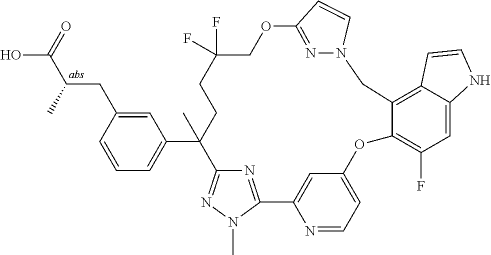 [SMILES: C[C@@H](Cc1cccc(C2(C)CCC(F)(F)COc3ccn(n3)Cc3c(c(F)cc4[nH]ccc34)Oc3ccnc(c3)-c3nc2nn3C)c1)C(=O)O] |
| 131B | 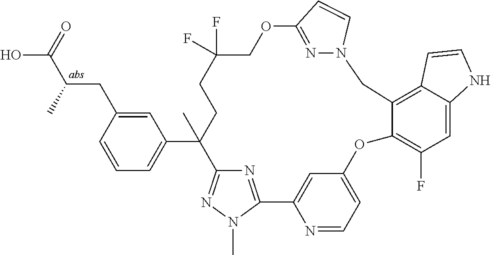 [SMILES: C[C@@H](Cc1cccc(C2(C)CCC(F)(F)COc3ccn(n3)Cc3c(c(F)cc4[nH]ccc34)Oc3ccnc(c3)-c3nc2nn3C)c1)C(=O)O] |
| 132A | 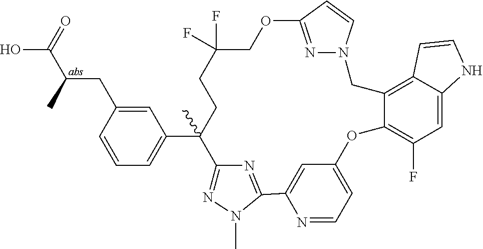 [SMILES: C[C@H](Cc1cccc(C2(C)CCC(F)(F)COc3ccn(n3)Cc3c(c(F)cc4[nH]ccc34)Oc3ccnc(c3)-c3nc2nn3C)c1)C(=O)O] |
| 132B | 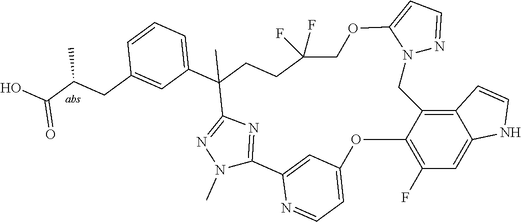 [SMILES: C[C@H](Cc1cccc(C2(C)CCC(F)(F)COc3ccnn3Cc3c(c(F)cc4[nH]ccc34)Oc3ccnc(c3)-c3nc2nn3C)c1)C(=O)O] |
| 133 | 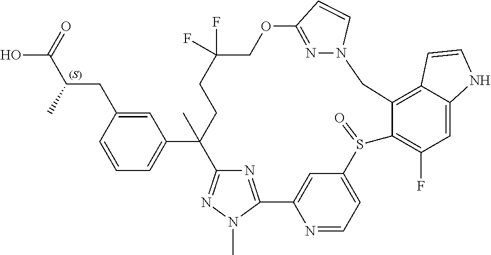 |
| 134A | 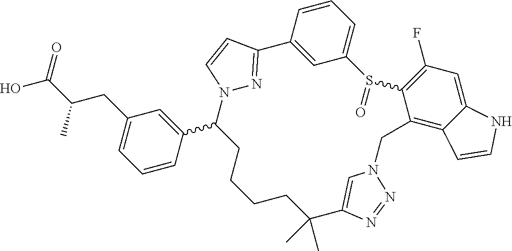 [SMILES: C[C@@H](Cc1cccc(C2CCCCC(C)(C)c3cn(nn3)Cc3c(c(F)cc4[nH]ccc34)S(=O)c3cccc(c3)-c3ccn2n3)c1)C(=O)O] |
| 134B | 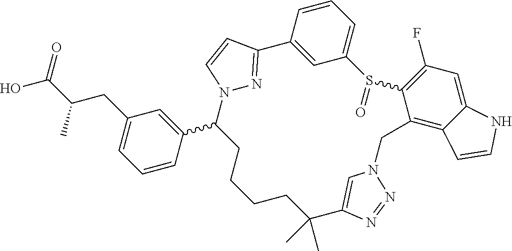 [SMILES: C[C@@H](Cc1cccc(C2CCCCC(C)(C)c3cn(nn3)Cc3c(c(F)cc4[nH]ccc34)S(=O)c3cccc(c3)-c3ccn2n3)c1)C(=O)O] |
| 135A | 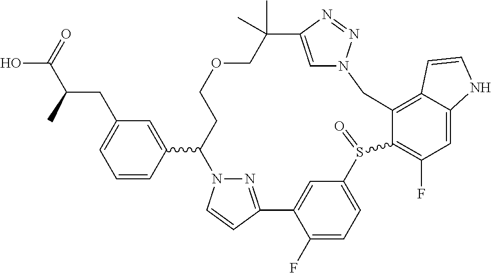 [SMILES: C[C@H](Cc1cccc(C2CCOCC(C)(C)c3cn(nn3)Cc3c(c(F)cc4[nH]ccc34)S(=O)c3ccc(F)c(c3)-c3ccn2n3)c1)C(=O)O] |
| 135B | 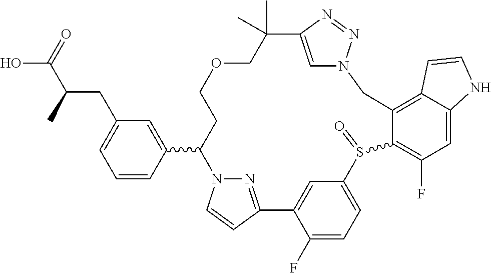 [SMILES: C[C@H](Cc1cccc(C2CCOCC(C)(C)c3cn(nn3)Cc3c(c(F)cc4[nH]ccc34)S(=O)c3ccc(F)c(c3)-c3ccn2n3)c1)C(=O)O] |
| 136 | 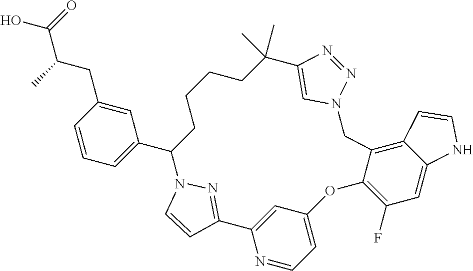 [SMILES: C[C@@H](Cc1cccc(C2CCCCC(C)(C)c3cn(nn3)Cc3c(c(F)cc4[nH]ccc34)Oc3ccnc(c3)-c3ccn2n3)c1)C(=O)O] |
| 137 | 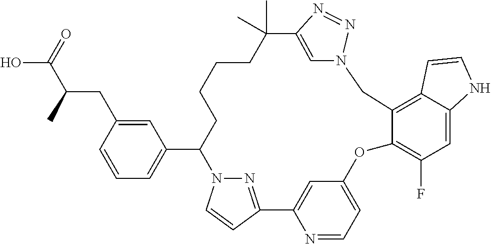 [SMILES: C[C@H](Cc1cccc(C2CCCCC(C)(C)c3cn(nn3)Cc3c(c(F)cc4[nH]ccc34)Oc3ccnc(c3)-c3ccn2n3)c1)C(=O)O] |
| 138 | 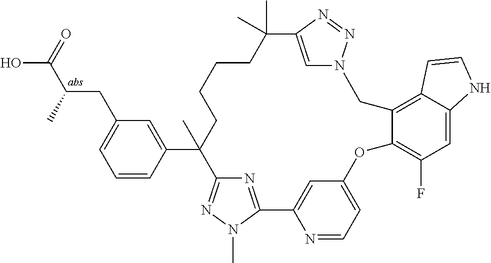 [SMILES: C[C@@H](Cc1cccc(C2(C)CCCCC(C)(C)c3cn(nn3)Cc3c(c(F)cc4[nH]ccc34)Oc3ccnc(c3)-c3nc2nn3C)c1)C(=O)O] |
| 139 | 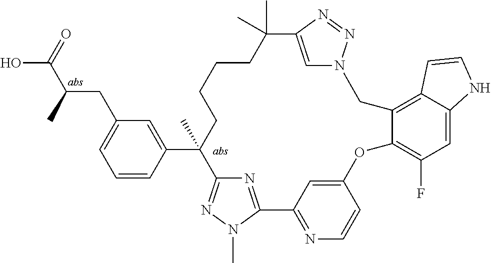 [SMILES: C[C@H](Cc1cccc([C@@]2(C)CCCCC(C)(C)c3cn(nn3)Cc3c(c(F)cc4[nH]ccc34)Oc3ccnc(c3)-c3nc2nn3C)c1)C(=O)O] |
| 140 | 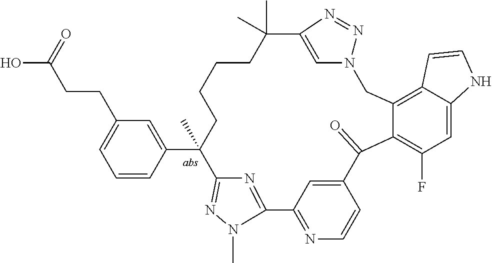 [SMILES: Cn1nc2nc1-c1cc(ccn1)C(=O)c1c(F)cc3[nH]ccc3c1Cn1cc(nn1)C(C)(C)CCCC[C@]2(C)c1cccc(CCC(=O)O)c1] |
| 141 | 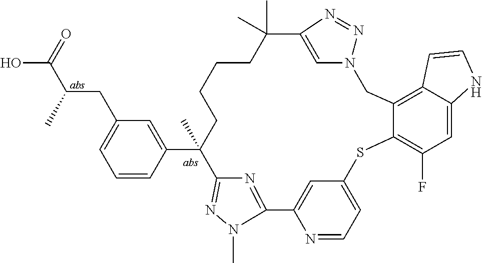 [SMILES: C[C@@H](Cc1cccc([C@@]2(C)CCCCC(C)(C)c3cn(nn3)Cc3c(c(F)cc4[nH]ccc34)Sc3ccnc(c3)-c3nc2nn3C)c1)C(=O)O] |
| 142 | 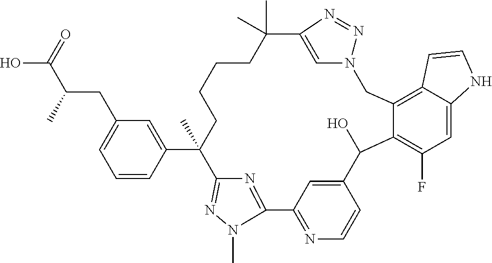 [SMILES: C[C@@H](Cc1cccc([C@@]2(C)CCCCC(C)(C)c3cn(nn3)Cc3c(c(F)cc4[nH]ccc34)C(O)c3ccnc(c3)-c3nc2nn3C)c1)C(=O)O] |
| 143 | 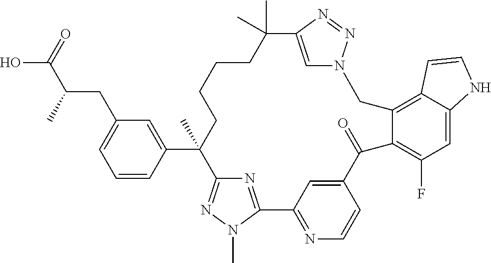 [SMILES: C[C@@H](Cc1cccc([C@@]2(C)CCCCC(C)(C)c3cn(nn3)Cc3c(c(F)cc4[nH]ccc34)C(=O)c3ccnc(c3)-c3nc2nn3C)c1)C(=O)O] |
| 144 | 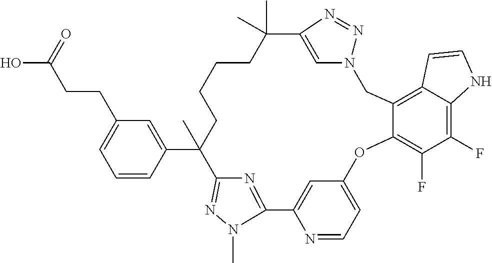 [SMILES: Cn1nc2nc1-c1cc(ccn1)Oc1c(F)c(F)c3[nH]ccc3c1Cn1cc(nn1)C(C)(C)CCCCC2(C)c1cccc(CCC(=O)O)c1] |
| 145 | 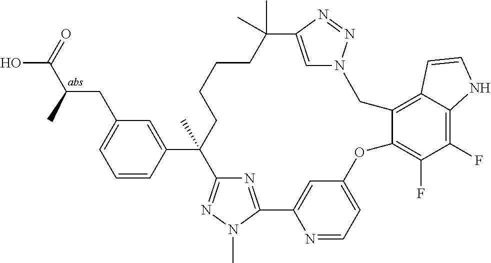 [SMILES: C[C@H](Cc1cccc([C@@]2(C)CCCCC(C)(C)c3cn(nn3)Cc3c(c(F)c(F)c4[nH]ccc34)Oc3ccnc(c3)-c3nc2nn3C)c1)C(=O)O] |
| 146 | 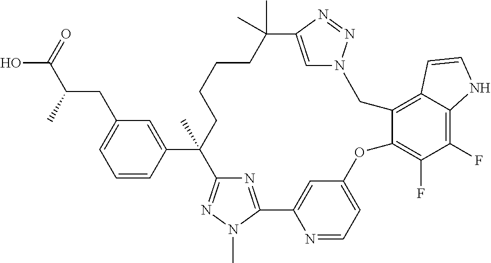 [SMILES: C[C@@H](Cc1cccc([C@@]2(C)CCCCC(C)(C)c3cn(nn3)Cc3c(c(F)c(F)c4[nH]ccc34)Oc3ccnc(c3)-c3nc2nn3C)c1)C(=O)O] |
| 147 | 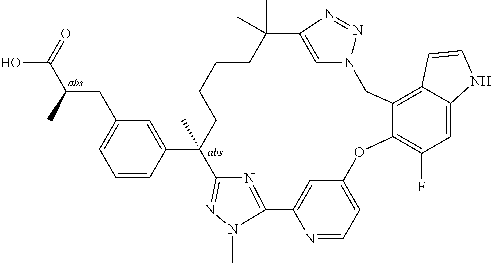 [SMILES: C[C@H](Cc1cccc([C@@]2(C)CCCCC(C)(C)c3cn(nn3)Cc3c(c(F)cc4[nH]ccc34)Oc3ccnc(c3)-c3nc2nn3C)c1)C(=O)O] |
| 148 | 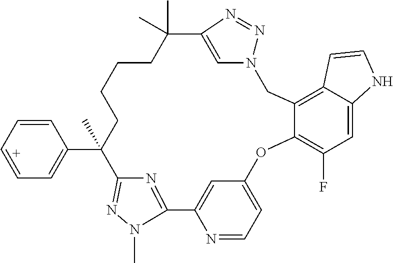 [SMILES: Cn1nc2nc1-c1cc(ccn1)Oc1c(F)cc3[nH]ccc3c1Cn1cc(nn1)C(C)(C)CCCC[C@]2(C)C1=CC=[C+]C=C1] |
| 149 | 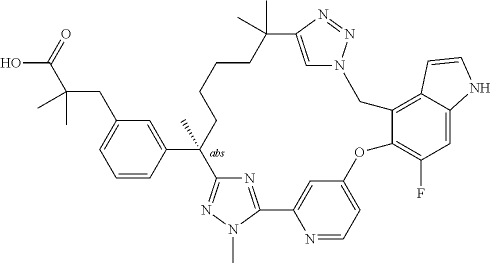 [SMILES: Cn1nc2nc1-c1cc(ccn1)Oc1c(F)cc3[nH]ccc3c1Cn1cc(nn1)C(C)(C)CCCC[C@]2(C)c1cccc(CC(C)(C)C(=O)O)c1] |
| 150 | 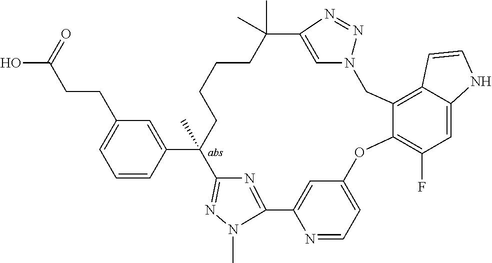 [SMILES: Cn1nc2nc1-c1cc(ccn1)Oc1c(F)cc3[nH]ccc3c1Cn1cc(nn1)C(C)(C)CCCC[C@]2(C)c1cccc(CCC(=O)O)c1] |
| 151 | 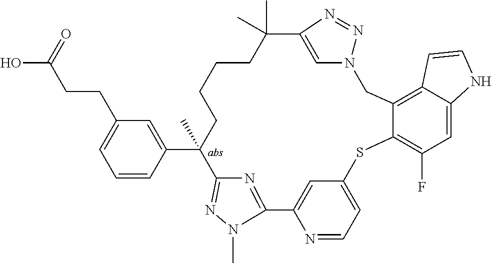 [SMILES: Cn1nc2nc1-c1cc(ccn1)Sc1c(F)cc3[nH]ccc3c1Cn1cc(nn1)C(C)(C)CCCC[C@]2(C)c1cccc(CCC(=O)O)c1] |
| 152 | 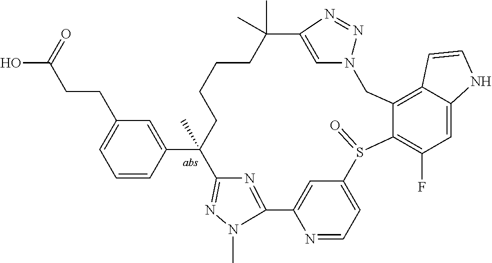 [SMILES: Cn1nc2nc1-c1cc(ccn1)S(=O)c1c(F)cc3[nH]ccc3c1Cn1cc(nn1)C(C)(C)CCCC[C@]2(C)c1cccc(CCC(=O)O)c1] |

or a pharmaceutically acceptable salt thereof.

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “haloalkyl” refers to a straight or branched alkyl group that is substituted with one or more halogen atoms.

The term “alkyl” as used herein is a branched or unbranched saturated hydrocarbon group having a specified number of carbon atoms. In some embodiments, alkyl refers to a branched or unbranched saturated hydrocarbon group having three carbon atoms (C₃). In some embodiments, alkyl refers to a branched or unbranched saturated hydrocarbon group having six carbon atoms (C₆). In some embodiments, the term “alkyl” includes, but is not limited to, methyl, ethyl, n-propyl, isopropyl, n-butyl, isobutyl, s-butyl, t-butyl, n-pentyl, isopentyl, s-pentyl, neopentyl, and hexyl.

As used herein, the term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group. The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring.

Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)_{0- 4}N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)o₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘}include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH2, —NHR^{∘}, —NR^{●}₂, or —NO₂, wherein each R^{●} is unsubstituted or where preceded by “halo” is substituted only with one or more halogens, and is independently C₁4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat and/or diagnose the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an CFTR-associated disease or disorder.

The terms “treat”, “treatment” or “treating” mean to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. Without being bound by any theory, in some embodiments, treating includes augmenting deficient CFTR activity. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein, “CFTR” means cystic fibrosis transmembrane conductance regulator. Defects in the function of the CFTR ion channel result from loss of function mutations of CFTR. Such mutations lead to exocrine gland dysfunction, abnormal mucociliary clearance, and cause cystic fibrosis. The most common CFTR mutation in Cystic Fibrosis (CF) patients leads to the specific deletion of three nucleotides of the codon for phenylalanine at position 508. This mutation, which is found in ˜70% of CF patients worldwide, is referred to as “ΔF508”. The ΔF508 mutation decreases the stability of the CFTR NBD1 domain and limits CFTR interdomain assembly. Since CF is an autosomal recessive disease, a CF patient harboring the ΔF508 CFTR mutation must also carry a second defective copy of CFTR. Approximately 2000 different CF-causing CFTR mutations have been identified in CF patients. CF patients harboring the ΔF508 CFTR mutation can be homozygous for that mutation (ΔF508/ΔF508). CF patients can also be ΔF508 heterozygous, if the second CFTR allele such patients carry instead contains a different CFTR loss of function mutation. Such CFTR mutations include, but are not limited to, G542X, G551D, N1303K, W1282X, R553X, R117H, R1162X, R347P, G85E, R560T, A455E, ΔI507, G178R, S549N, S549R, G551S, G970R, G1244E, S1251N, S1255P, and G1349D.

As used herein, the term “CFTR modulator” refers to a compound that increases the activity of CFTR. In certain aspects, a CFTR modulator is a CFTR corrector or a CFTR potentiator or a dual-acting compound having activities of a corrector and a potentiator.

As used herein, the term “CFTR corrector” refers to a compound that increases the amount of functional CFTR protein to the cell surface and thus enhances CFTR channel function. The CFTR correctors partially “rescue” misfolding of CFTR, thereby enabling the maturation and functional expression of CFTR protein harboring a CF causing mutation on the cell surface. Examples of correctors include, but are not limited to, VX-809, VX-661, VX-152, VX-440, VX-983, and GLPG2222. Such compounds may interact directly with CFTR protein, modifying its folding and conformational maturation during synthesis.

As used herein, the term “CFTR potentiator” refers to a compound that increases the ion channel activity of CFTR protein located at the cell surface, resulting in enhanced ion transport. CFTR potentiators repair the defective channel functions caused by mutations. Examples of potentiators include, but are not limited to, ivacaftor (VX770), deuterated ivacaftor (CPT 656), genistein and GLPG1837.

As used herein, the term “CFTR pharmacological chaperone” (PC) refers to compounds that stabilize the CFTR protein in its native state by binding directly to the protein.

As used herein, the term “CFTR proteostasis regulator” (PR) refers to compounds that enhance the protein folding efficiency within the cell. PRs can alter the activity of transcriptional, folding and/or membrane trafficking machinery, as well as impeding the degradation of partially folded, but functional, conformers at the endoplasmic reticulum (ER) or plasma membrane.

As used herein, “CFTR disease or condition” refers to a disease or condition associated with deficient CFTR activity, for example, cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, dry eye disease, protein C deficiency, A-beta.-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (A) and a pharmaceutically acceptable carrier, adjuvant, or vehicle. In some embodiments, the amount of compound in compositions contemplated herein is such that is effective to measurably modulate CFTR, or a mutant thereof, in a biological sample or in a patient.

In certain embodiments, the amount of compound in compositions of this disclosure is such that is effective to measurably modulate CFTR, or a mutant thereof, in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, the amount of compound in compositions contemplated herein is such that is effective to measurably modulate a protein, particularly at CFTR, or a mutant thereof, in a biological sample or in a patient. In certain embodiments, the amount of compound in compositions of this disclosure is such that is effective to measurably modulate CFTR, or a mutant thereof, in a biological sample or in a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (A) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (A) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (A) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (A) may also be administered topically, especially when the target of treatment includes areas or organs readily accessible by topical application, including diseases of the eye, the skin, or the lower intestinal tract. Suitable topical formulations are readily prepared for each of these areas or organs.

In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (A) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

As discussed above, CFTR is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate and thiocyanate into and out of the cell. The most frequent CFTR mutation is the in-frame deletion of phenylalanine at residue 508 (ΔF508) in the first nucleotide binding domain (NBD1). The mutation has several deleterious effects on the production of CFTR in the ER, its correct folding, its movement to the plasma membrane and its normal function as an ion channel for the cell.

One such negative effect is that the NBD1 domain is partially or mis-folded which is recognized within the cell as an aberrant protein and tagged for disposal by ER-associated degradation (ERAD) via the ubiquitin-proteasome system (UPS). Should a partially or mis-folded CFTR protein emerge from the ER, the protein must travel to the plasma membrane through complex glycosylation in the Golgi compartment and be functionally inserted. In wild-type CFTR, only 20-40% of CFTR reaches the plasma membrane, indicating that CFTR has energetic instability of individual NBDs, a slow domain assembly, and relatively fast ERAD kinetics which all contribute to inefficient folding and sensitize CFTR to structural perturbations by mutations.

In wild-type CFTR, the NBD1 domain folds co-translationally while other domains fold post-translationally. Mutated ΔF508 CFTR has impaired NBD1 folding but its backbone structure and thermodynamic stability are similar to wild-type CFTR. With delayed folding kinetics, mutated ΔF508 CFTR NBD1 has an increased folding activation energy. Lack of proper folding results in hydrophobic residues being exposed to the surface of NBD1 which causes aggregation with other CFTR proteins. Thus, the aggregation temperature of mutated CFTR drops from 41° C. to 33° C. This level of instability creates a greater percentage of mis-folded mutant CFTR at physiological temperature (37° C. in humans). Mutant CFTR suffers from both kinetic and thermodynamic folding defects. CFTR stabilizers can address these folding defects, but complete energetic correction of mutant NBD1 folding has been shown to not result in the CFTR biosynthetic processing, underscoring the need for interface stability as well.

The disclosed CFTR correctors can interact with the NBD domain to stabilize the correct folded position R, such that CFTR is not labeled for elimination from the cell. The preservation of correct folding enables CFTR to function as a chloride ion channel at wild-type levels. In some embodiments, disclosed CFTR correctors can enhance the performance of wild-type CFTR.

CFTR stabilizers can function in combination with other therapeutic agents such as CFTR correctors that promote Δ508 CFTR exit from the ER and accumulation in the plasma membrane. Increasing the amount of CFTR cell surface expression can result in improved chloride conductance following channel activation by both potentiators and a cAMP agonist. Thus, disclosed herein are combinations of CFTR stabilizers with CFTR correctors and potentiators, optionally with cAMP agonists or another therapeutic agent as described below.

Disclosed herein are methods of treating deficient CFTR activity in a cell, comprising contacting the cell with a compound of Formula (A), or a pharmaceutically acceptable salt thereof. In certain embodiments, contacting the cell occurs in a subject in need thereof, thereby treating a disease or disorder mediated by deficient CFTR activity.

Also, disclosed herein are methods of treating a disease or a disorder mediated by deficient CFTR activity comprising administering a compound of Formula (A) or a pharmaceutically acceptable salt thereof. In some embodiments, the subject is a mammal, preferably a human. In some embodiments, the disease is associated with the regulation of fluid volumes across epithelial membranes, particularly an obstructive airway disease such as CF or COPD.

Such diseases and conditions include, but are not limited to, cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear plasy, Pick's disease, several polyglutamine neurological disorders, Huntington's, spinocerebullar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

Such diseases and conditions include, but are not limited to, cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome. In some embodiments, the disease is cystic fibrosis.

Provided herein are methods of treating cystic fibrosis, comprising administering to a subject in need thereof, a compound as disclosed herein or a pharmaceutically acceptable salt thereof. Also provided herein are methods of lessening the severity of cystic fibrosis, comprising administering to a subject in need thereof, a compound as disclosed herein or a pharmaceutically acceptable salt thereof. In some embodiments, the subject is a human. In some embodiments, the subject is at risk of developing cystic fibrosis, and administration is carried out prior to the onset of symptoms of cystic fibrosis in the subject.

Provided herein are compounds as disclosed herein for use in treating a disease or condition mediated by deficient CFTR activity. Also provided herein are uses of a compound as disclosed herein for the manufacture of a medicament for treating a disease or condition mediated by deficient CFTR activity.

Provided herein are kits for use in measuring the activity of CFTR or a fragment thereof in a biological sample in vitro or in vivo. The kit can contain: (i) a compound as disclosed herein, or a pharmaceutical composition comprising the disclosed compound, and (ii) instructions for: a) contacting the compound or composition with the biological sample; and b) measuring activity of said CFTR or a fragment thereof. In some embodiments, the biological sample is biopsied material obtained from a mammal or extracts thereof; blood, saliva, urine, feces, semen, tears, other body fluids, or extracts thereof. In some embodiments, the mammal is a human.

### Combination Treatments

As used herein, the term “combination therapy” means administering to a subject (e.g., human) two or more CFTR modulators, or a CFTR modulator and an agent such as antibiotics, ENaC inhibitors, GSNO (S-nitrosothiol, s-nitroglutathione) reductase inhibitors, and a CRISPR Cas correction therapy or system (as described in US 2007/0022507 and the like). In some embodiments, combination therapy includes administration of a compound described herein with a compound that modulates CFTR protein or ABC protein activities (e.g., as described in WO2018167690A1 and the like)

In certain embodiments, the method of treating a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

In certain embodiments, the method of preventing a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

Additional therapeutic agents include, for example, ENaC inhibitors, mucolytic agents, modulators of mucus rheology, bronchodilators, antibiotics, anti-infective agents, anti-inflammatory agents, ion channel modulating agents, therapeutic agents used in gene or mRNA therapy, agents that reduce airway surface liquid and/or reduce airway surface PH, CFTR correctors, and CFTR potentiators, or other agents that modulate CFTR activity. Other therapeutics include liposomal composition components such as those described in WO2012/170889, hybrid oligonucleotides that facilitate RNA cleavage such as those described in WO2016/130943, and single stranded oligonucleotides that modulate gene expression as described in WO2016/130929.

In some embodiments, at least one additional therapeutic agent is selected from one or more CFTR modulators, one or more CFTR correctors and one or more CFTR potentiators.

Non-limiting examples of additional therapeutics include VX-770 (Ivacaftor), VX-809 (Lumacaftor, 3-(6-(I-(2,2-5 difluorobenzo[d][1,3]dioxol-5-yl)cyclopropanecarboxamido)-3-methylpyridin-2-yl) benzoic acid, VX-661 (Tezacaftor, I-(2,2-difluoro-1, 3-benzodioxol-5-yl)-N—[I-[(2R)-2,3-dihydroxypropyl]-6-fluoro-2-(2-hydroxy-1, I-dimethylethyl)-1H-indol-5-yl]-cyclopropanecarboxamide), VX-983, VX-152, VX-440, VX-445, VX-659, VX-371, Orkambi, Ataluren (PTC 124) (3-[5-(2-fluorophenyl)-1, 2,4-oxadiazol-3-yl]benzoic acid), PTI-130 (Proteostasis), PTI-801, PTI-808, PTI-428, N91115.74 (cavosonstat), QBW251 (Novartis) compounds described in WO2011113894, compounds N30 Pharmaceuticals (e.g., WO 2014/186704), deuterated ivacaftor (e.g., CTP-656 or VX-561), GLPG 2222, GLPG2451, GLPG3067, GLPG2851, GLPG2737, GLPG 1837 (N-(3-carbamoyl-5,5,7,7-tetramethyl-5,7-dihydro-4H-thieno[2,3-c]pyran-2-yl)-1H-pyrazole-5-carboxamide), GLPG 2665 (Galapagos), FDL 169 (Flatley Discovery lab), FDL 176, FDL438, FDL304, FD2052160, FD1881042, FD2027304, FD2035659, FD2033129, FD1860293, CFFT-Pot01, CFFT-Pot-02, P-1037, glycerol, phenylbutyrate, and the like.

Non-limiting examples of additional therapeutics include compounds disclosed in US Patent Application Nos. 62/944,141, 62/944,158 and 62/944,188, each of which is incorporated by reference in its entirety.

Non-limiting examples of anti-inflammatory agents are N6022 (3-(5-(4-(1H-imidazol-I-yl)10 phenyl)-I-(4-carbamoyl-2-methylphenyl)-′H-pyrrol-2-yl) propanoic acid), Ibuprofen, Lenabasum (anabasum), Acebilustat (CTX-4430), LAU-7b, POL6014, docosahexaenoic acid, alpha-1 anti-trypsin, sildenafil. Additional therapeutic agents also include, but are not limited to a mucolytic agent, a modifier of mucus rheology (such as hypertonic saline, mannitol, and oligosaccharide based therapy), a bronchodilator, an anti-infective (such as tazobactam, piperacillin, rifampin, meropenum, ceftazidime, aztreonam, tobramycin, fosfomycin, azithromycin, amitriptyline, vancomycin, gallium and colistin), an anti-infective agent, an anti-inflammatory agent, a CFTR modulator other than a compound of the present disclosure, and a nutritional agent. Additional therapeutic agents can include treatments for comorbid conditions of cystic fibrosis, such as exocrine pancreatic insufficiency which can be treated with Pancrelipase or Liprotamase.

Examples of CFTR potentiators include, but are not limited to, Ivacaftor (VX-770), CTP-656, NVS-QBW251, FD1860293, GLPG2451, GLPG1837, and N-(3-carbamoyl-5,5,7,7-tetramethyl-5,7-dihydro-4H-thieno[2,3-c]pyran-2-yl)-1H-pyrazole-5-carboxamide. Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, WO2013038390, WO2014180562, WO2015018823, and U.S. patent application Ser. Nos. 14/271,080, 14/451,619 and 15/164,317.

Non-limiting examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanec arboxamide (VX-661), VX-983, GLPG2222, GLPG2665, GLPG2737, VX-152, VX-440, FDL169, FDL304, FD2052160, and FD2035659. Examples of correctors are also disclosed in US20160095858A1, and U.S. application Ser. Nos. 14/925,649 and 14/926,727.

In certain embodiments, the additional therapeutic agent is a CFTR amplifier. CFTR amplifiers enhance the effect of known CFTR modulators, such as potentiators and correctors. Examples of CFTR amplifier include PTI130 and PTI-428. Examples of amplifiers are also disclosed in publications: WO2015138909 and WO2015138934.

In certain embodiments, the additional therapeutic agent is an agent that reduces the activity of the epithelial sodium channel blocker (ENaC) either directly by blocking the channel or indirectly by modulation of proteases that lead to an increase in ENaC activity (e.g., serine proteases, channel-activating proteases). Exemplary of such agents include camostat (a trypsin-like protease inhibitor), QAU145, 552-02, GS-9411, INO-4995, Aerolytic, amiloride, AZD5634, and VX-371. Additional agents that reduce the activity of the epithelial sodium channel blocker (ENaC) can be found, for example, in PCT Publication No. WO2009074575 and WO2013043720; and U.S. Pat. No. 8,999,976.

In one embodiment, the ENaC inhibitor is VX-371.

In one embodiment, the ENaC inhibitor is SPX-101 (S18).

In certain embodiments, the combination of a compound of Formula (A), with a second therapeutic agent may have a synergistic effect in the treatment of cancer and other diseases or disorders mediated by adenosine. In other embodiments, the combination may have an additive effect.

### EXEMPLIFICATION

### Abbreviations

Boc: tert-butyloxycarbonyl
DEA: diethyl amine
DBU: 1,8-Diazabicyclo[5.4.0]undec-7-ene
DMSO: dimethyl sulfoxide
dppf: 1,1′-Bis(diphenylphosphino)ferrocene
DTT: dithiothreitol
ESI: electron spray ionization
HATU: 1-[Bis(dimethylamino)methylene]-1H-1,2,3-triazolo[4,5-b]pyridinium 3-oxide hexafluorophosphate
HPLC: high performance liquid chromatography
LC-MS: liquid chromatography-mass spectrometry
NIS: N-iodosuccinimide
Pd/C: Palladium on carbon
SFC: supercritical fluid chromatography
TBS: tert-Butyldimethylsilyl
TIPS: Triisopropylsilyl
THF: tetrahydrofuran
THP: tetrahydropyran
Ts: tosyl

### General Procedures

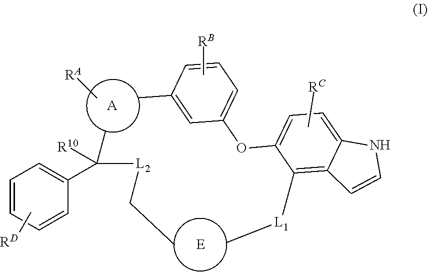

The compounds of the present disclosure can be better understood in connection with the following synthetic schemes and methods which illustrate means by which the compounds of the Formula (I) can be prepared. The compounds of this disclosure can be prepared by a variety of synthetic procedures illustrated in Schemes I to VII.

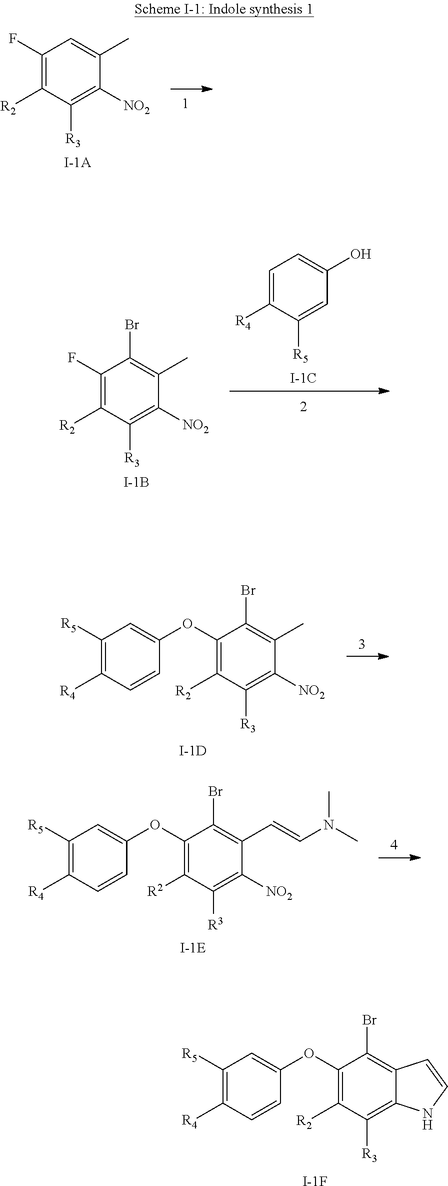

The intermediate I-1F may be prepared as illustrated in Scheme I-i. Properly substituted methyl nitrobenzene (I-1A) is brominated (step 1) to give bromide I-1B. Compound I-1B is condensed with phenol I-1C (step 2) to give the ether I-1D. Treatment of I-1D with N,N-dimethylformamide dimethyl acetal (step 3) to form 1-1E. Reductive-cyclization (step 4) of 1-1E results in the intermediate I-1F.

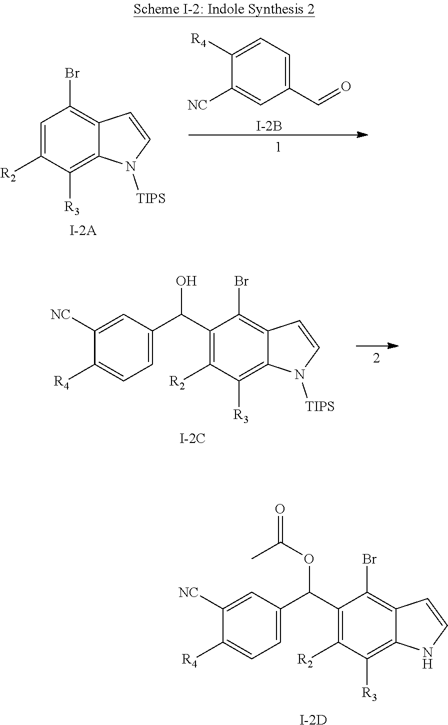

The bromo indole intermediate I-2D may be synthesized according to Scheme I-2. Triisopropylsilyl protected indole I-2A is deprotonated with a strong base like lithium diisopropylamide or lithium bis(trimethylsilyl)amide, then treated with aldehyde I-2B (step 1) to form I-2C. Acetyl protection results in I-2D.

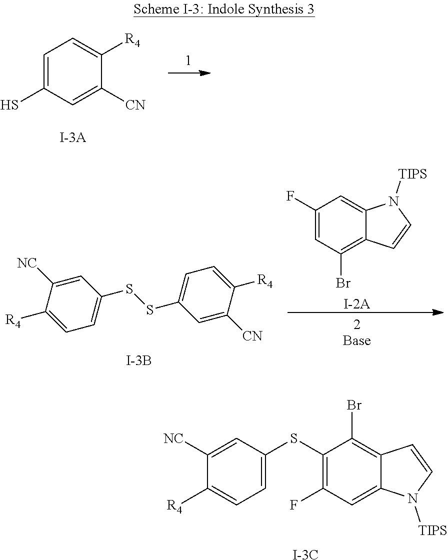

A similar method is used to prepare intermediate I-3C. Thiol I-3A is oxidized (step 1) to dithioether I-3B, which is then reacted with the regio-specific deprotonated indole I-2A (step 2) to afford the Intermediate I-3C.

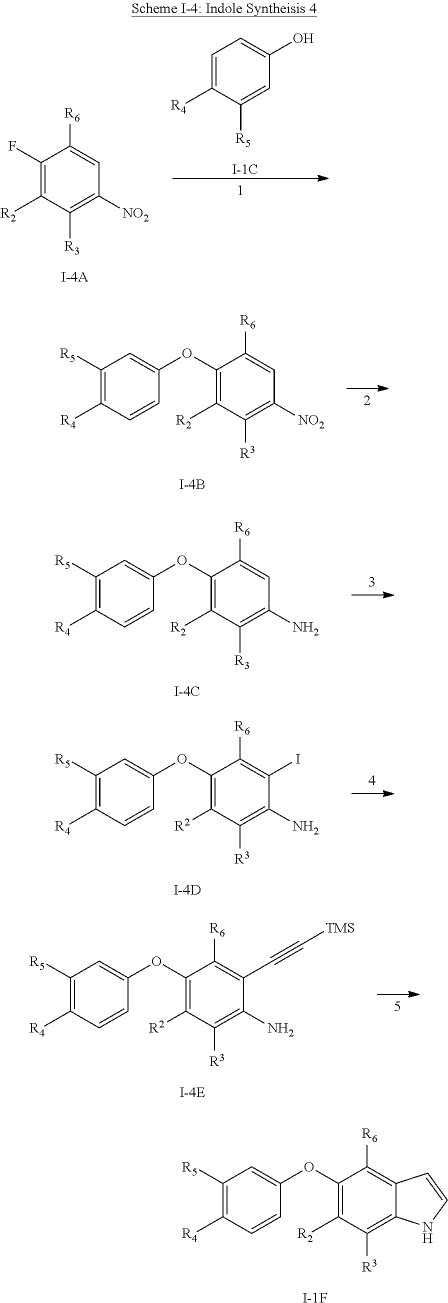

Scheme I-4 describes an alternative method to prepare intermediate I-1F. Intermediate I-4A is condensed with phenol I-1C (step 1) to yield intermediate I-4B. Reduction of the nitro group into amine (step 2) affords I-4C, to which iodine is introduced (step 3) to give intermediate I-4D. This intermediate is then coupled with protected acetylene to yield intermediate I-4E (step 4). Cyclization of I-4E (step 5) yields the key intermediate I-1F.

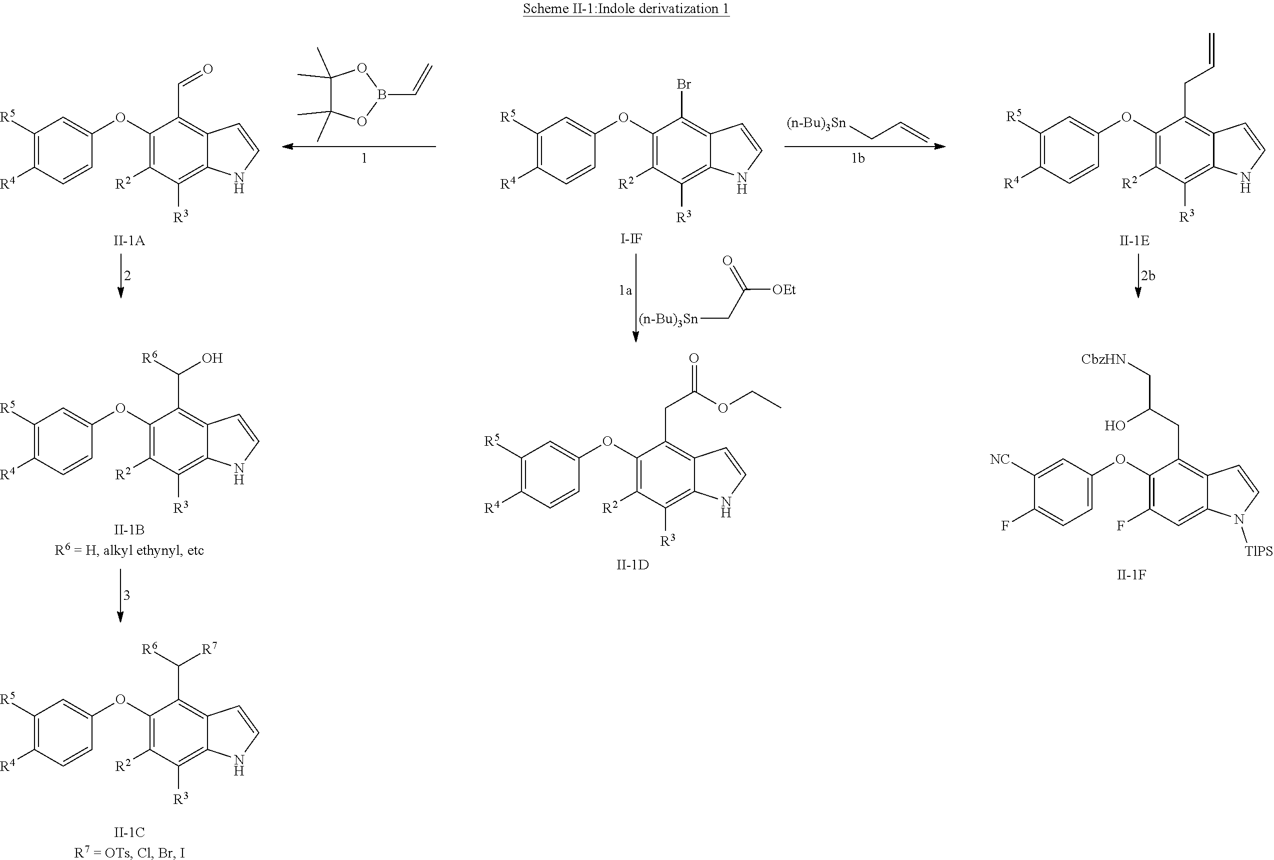

The indole I-1F can be further derivatized as shown in Scheme 11-1. The intermediate I-1F is coupled with vinyl boronic ester to derive an alkene which undergoes oxidative cleavage to yield aldehyde II-1A (step 1). Reduction or alkyl lithium or alkyl Grignard addition to the aldehyde gives an alcohol II-1B (Step 2). The resultant hydroxyl can be further derivatized as a leaving group, such as halogen or tosylate, and becomes ready for coupling (described later). Certain side chains at C4 of the indole can also be installed via a Stille coupling. For example, in Scheme H1-1, bromide I-1F is coupled (Step 1a) with Stille reagent to obtain an ester I-1D. Further to this call, the intermediate I-1F can also be coupled with organic tin reagent. For example, I-1F is coupled with allyl(tributyl)stannane catalyzed by lithium chloride and bis-(triphenylphosphine)palladium(II) chloride (Step 1b) to give three carbon chain with an alkene functional group (I-E) which is further derivatized (step 2b) into a proper coupling partner, such as II-1F.

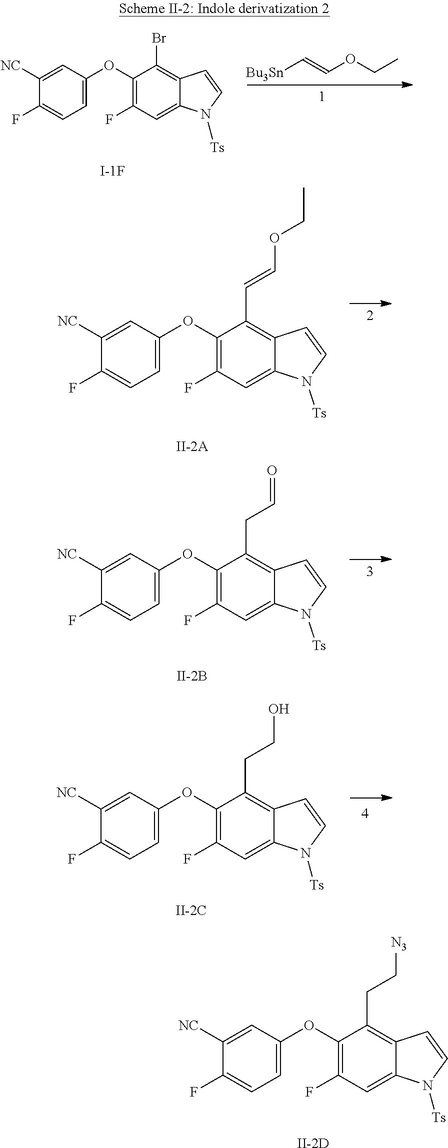

Following this direction further, I-1F is coupled with an organo-tin agent (Step 1) to give alkyl derivative II-2A which is converted into an aldehyde II-2B (Step 2). After reduction (Step 3) and activation using the proper agent, such as tosyl, the alcohol is converted into azide (II-2D) (Step 4) which can be used for the coupling reaction (see later).

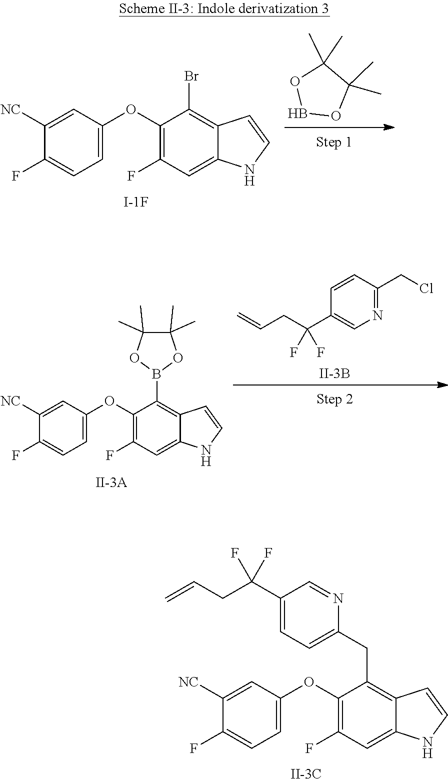

Bromide I-1F can also be converted into a Suzuki coupling agent. As shown in the example in Scheme 11-3, I-1F is coupled with boronic ester (Step 1) to give the Suzuki agent (II-3A). This agent is extremely versatile to couple with different partners. For example, II-3A can be coupled with chloride II-3B to give II-3C.

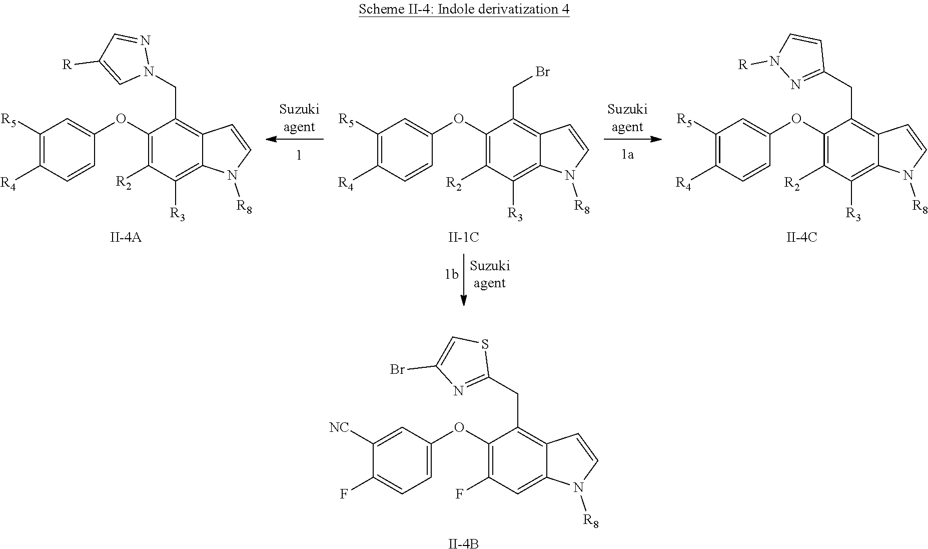

The derivative II-1C (R6═H, R7=Br) is further derivatized into different agents for the next step coupling reactions. As shown in Scheme II-4, the bromide II-1C is coupled with corresponding Suzuki agents (Step 1, 1a and 1b) to give heteroaryl derivatives, II-4A, II-4B, II-4C.

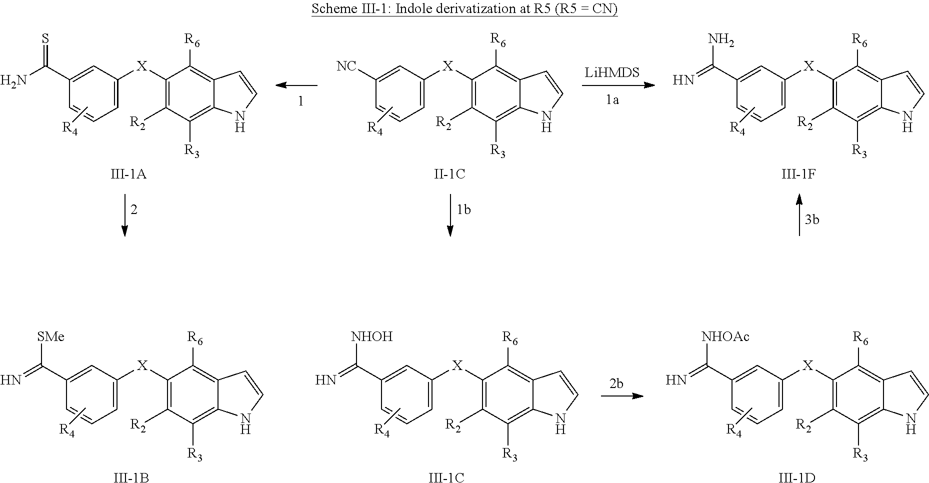

Methyl benzimidothioate (III-1B) may be derived from a 2-step sequence as shown in Scheme III-1. Nitrile derivative (II-1C) is converted to thioamide (III-1A) (step 1). The resultant thioamide is treated with an active methyl source such as iodomethane to obtain methyl benzimidothioate (III-1B) (step 2). Similarly, the intermediate amidine (III-1F) can be prepared from corresponding nitrile II-1C in one step when treated with lithium bis(trimethylsilyl)amide (step 1a). Alternatively, amidine III-1F may be prepared from a three-step sequence. Addition of hydroxylamine to nitrile II-1C results in hydoxyamidine (III-1C) (step 1b), acetylation (step 2b), followed by hydrogenation (Step 3b) to afford amidine III-1F.

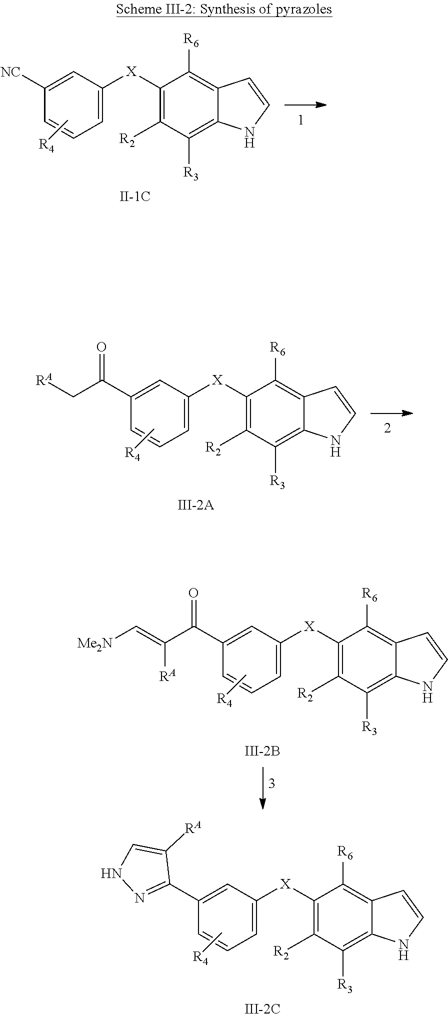

Intermediate III-2C may be synthesized through a three-step sequence (Scheme III-2). The nitrile II-1C or another related precursor is converted into the ketone III-2A (Step 1). This ketone is condensed with dimethylformamide dimethyl acetal yields intermediate III-2B (Step 2). The resulting imine is cyclized with hydrazine to form pyrazole III-2C (step 3).

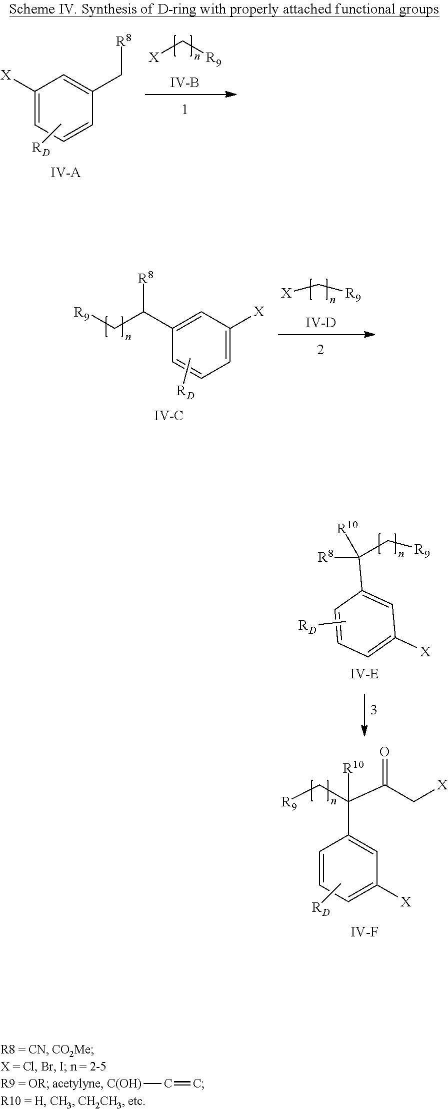

Scheme IV describes the synthesis of the key intermediate IV-F from readily available starting material IV-A. In Step 1, alkylation is catalyzed by a strong base, such as LDA, to form Intermediate IV-C. The process is repeated with another alkylating agent IV-D (Step 2) to yield intermediate IV-E. This intermediate is transformed into a halo-alkyl ketone through known chemistry and depends on the nature of R⁸ (Step 3) to derive the key intermediate IV-F.

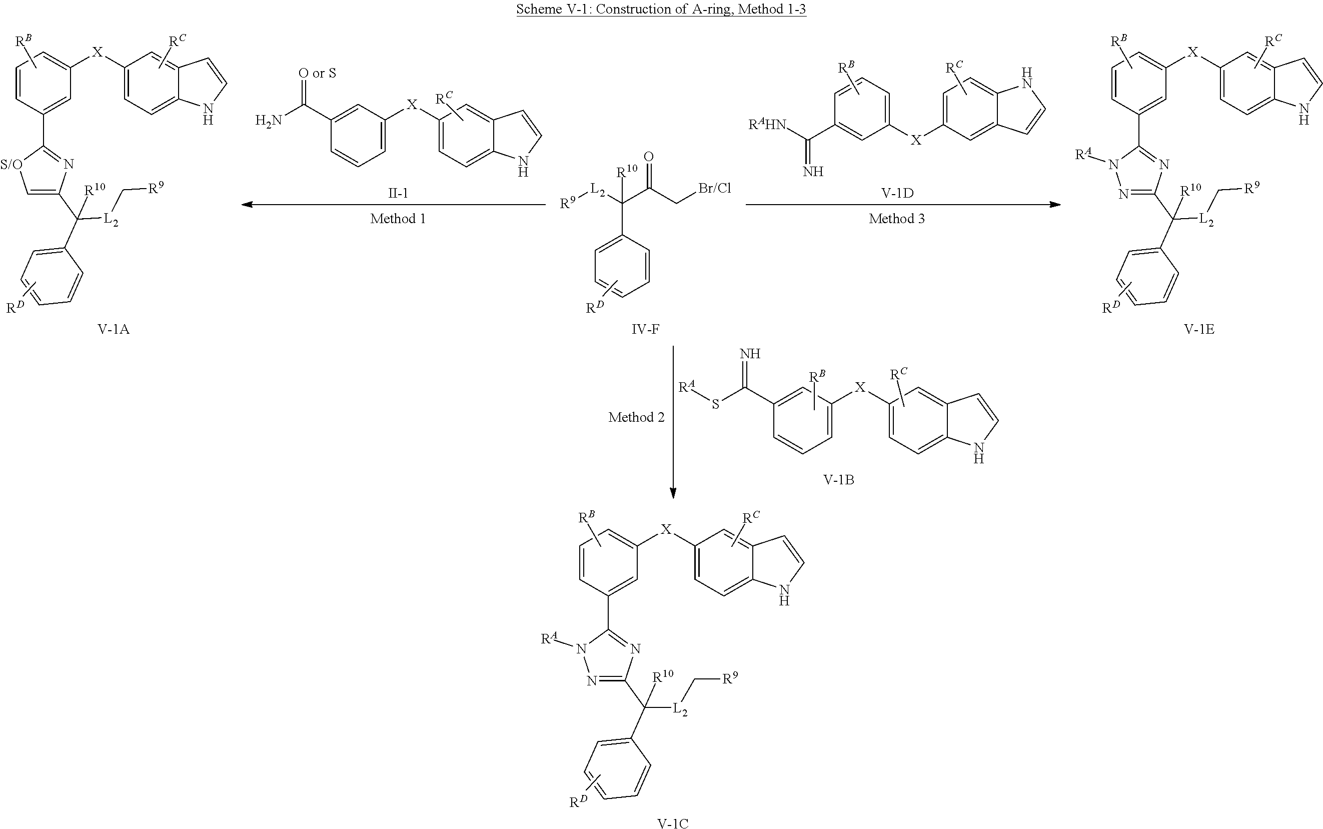

The substituted or unsubstituted hetero-aryl intermediate V-1 (A, C, E) is prepared through Method 1-3 as shown in Scheme V-1. In Method 1, halo-alkyl ketone (IV-F) is condensed with indole intermediate II-1 (A, B, C), selected based on the requirement of the reaction and the final compound, to give V-1A. Similarly, halo-alkyl ketone IV-F is condensed with alkylthio-imidine (V-1B) to give the intermediate V-1C (Method 2). Method 3 highlights the condensation of halo-alkyl ketone IV-F with imidine V-1D to give intermediate V-1E.

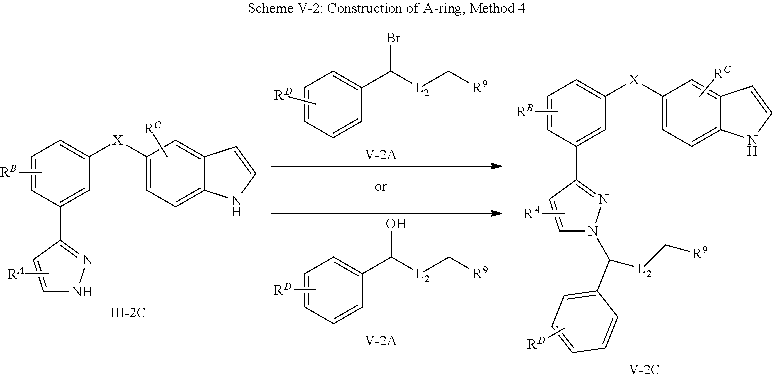

A-ring intermediate with the proper attached functional groups V-2C is synthesized via alkylation of intermediate III-2C with the proper alkylating agent V-2A or V-2B at ambient or elevated temperature and catalyzed by a base.

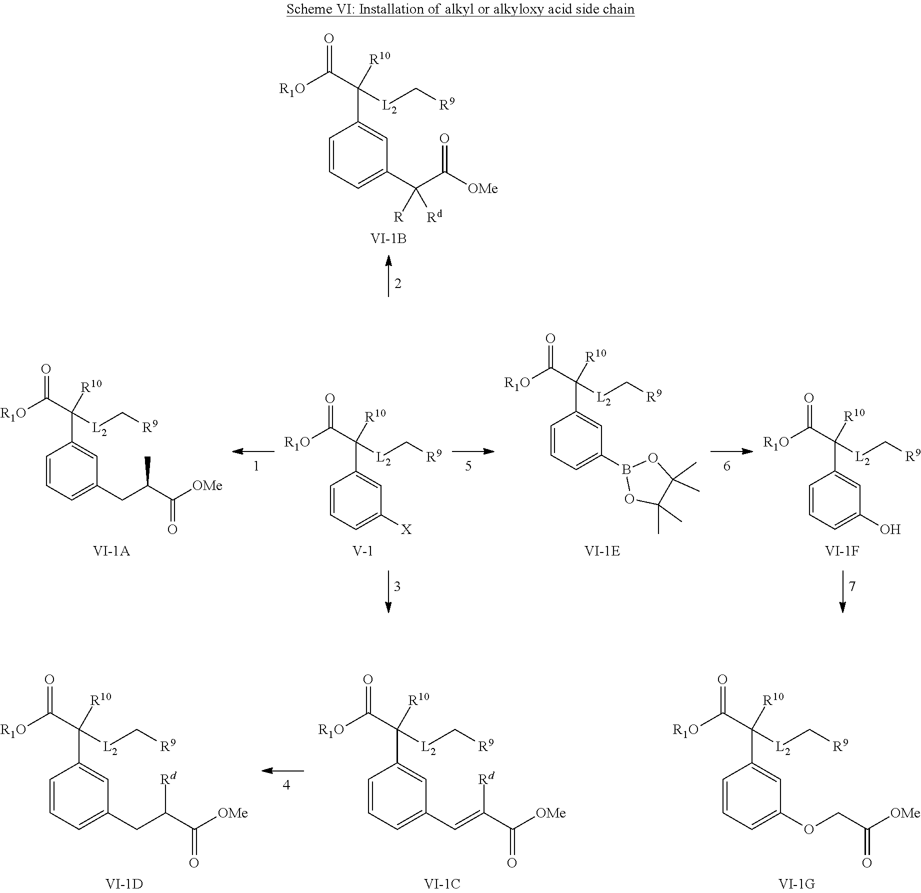

The installation of alkyl acid side chain is illustrated in Scheme VI. This sequence may start from the halide intermediates, such as IV (C, D, F) or V-1 (A, C, E). For example, Negishi coupling or other related coupling reactions of V-1 with a proper zinc agent gives VI-1A (step 1 or 2) and VI-1B. Intermediate V-1 is condensed with vinyl analog under Heck reaction conditions (step 3) followed by hydrogenation (step 4) affords intermediate VI-1D. Alkyloxy analogue VI-1G is prepared by a three-step sequence. First, the halide intermediate V-1 is converted into boronic ester (VI-1E) (step 5), followed by oxidation to a phenol analog (VI-1F) (step 6). Finally, the phenol analog is coupled with a proper halo-methyl ester (step 7).

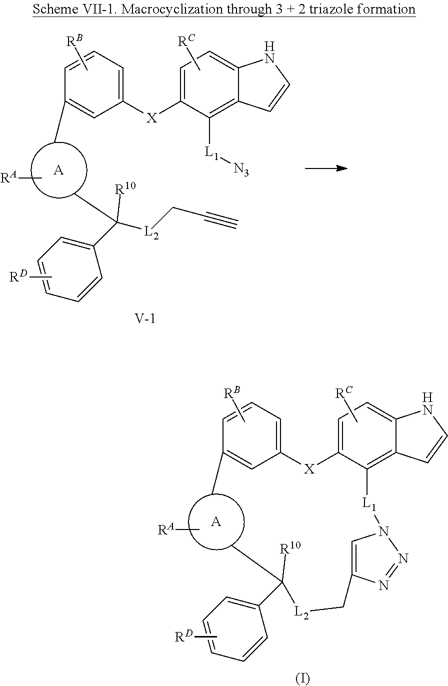

Macrocyclization may be achieved through 3+2 triazole formation. As illustrated in Scheme VII-1, the acetylene analog V-1 is heated in an inert solvent under relatively diluted conditions to yield triazole as the final designed compound of formula (I).

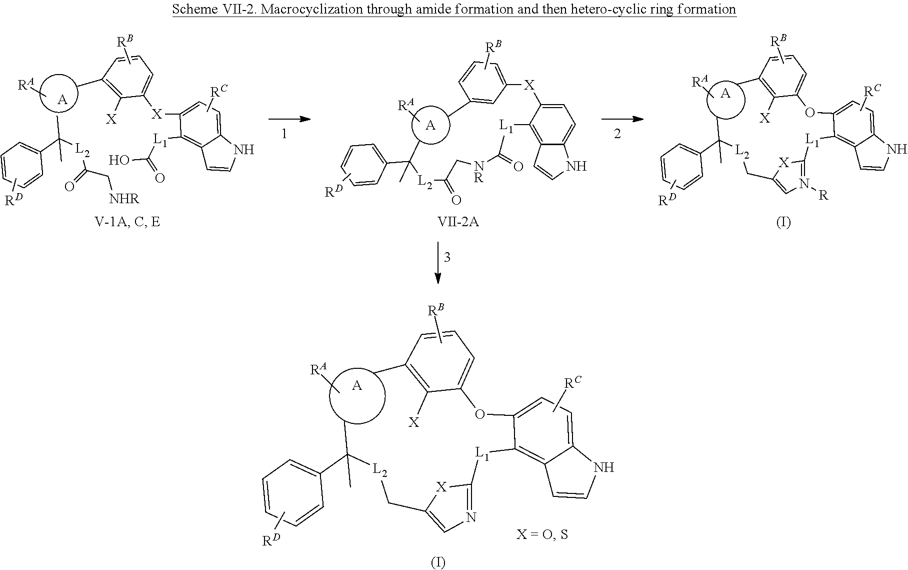

Macrocyclization may be achieved from precursor V-1 (A, C, E) via a macro-amidation reaction to obtain intermediate VII-2A (step 1), followed by cyclization (step 2, or 3) to form a hetero-aryl final compound of the formula (I). If Lawesson's reagent is applied to precursor VII-2A in the cyclization, thiazole is produced (I, X=S).

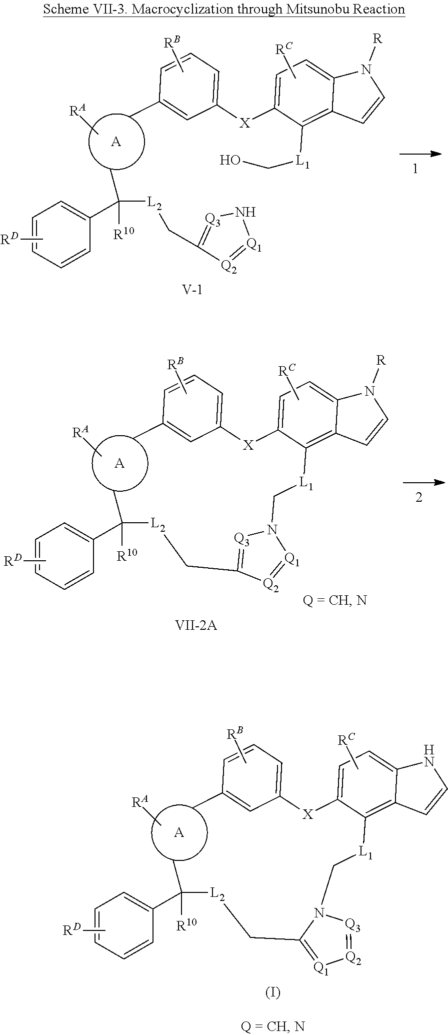

Scheme VII-3 illustrates the macrocycle formation via an alkylation reaction, such as the Mitsunobu reaction (step 1), of the precursor V-1 to obtain, after removal of the protecting group (step 2) the desired final compound of the formula (I).

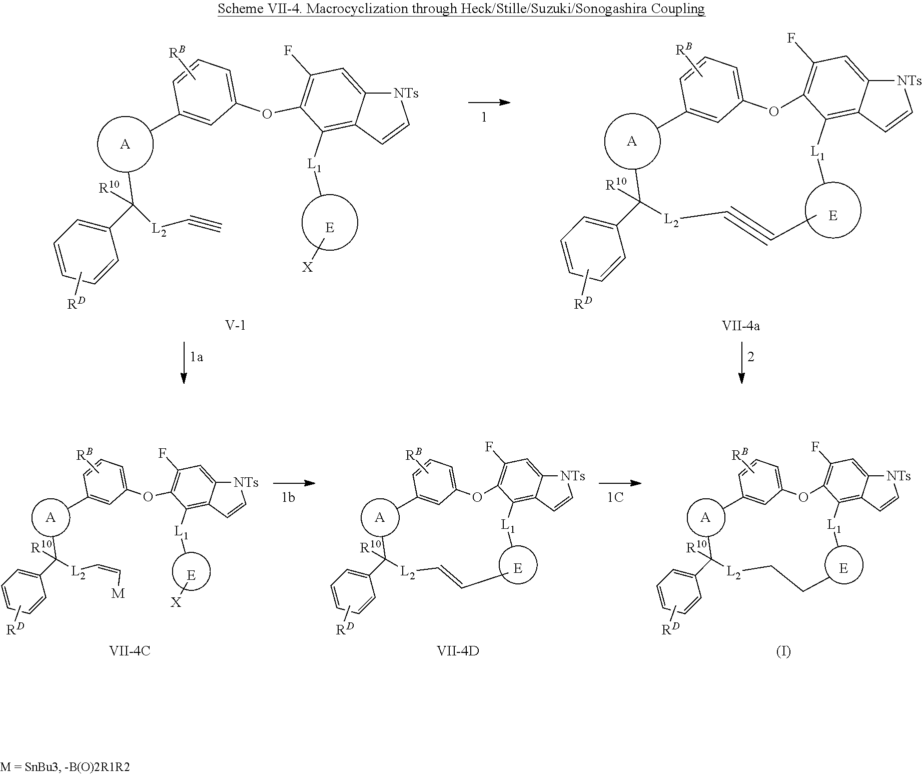

Heck/Stille/Suzuki/Sonogashira coupling is also successfully applied in the macrocyclization reaction as illustrated in Scheme VII-4. In step 1, acetylene functional group in V-1 is coupled with halo functional group in ring E to yield Intermediate VII-4A. After hydrogenation and removal of the protecting group, the final compound of formula (I) is obtained. In step 1a, V-1 is converted into a metallic intermediate VII-4C. This metallic intermediate is coupled with a proper functional group, such as a halo group, in ring-E to form Intermediate VII-4D. After hydrogenation and removal of the protection group, the designed final compound of formula (I) is realized.

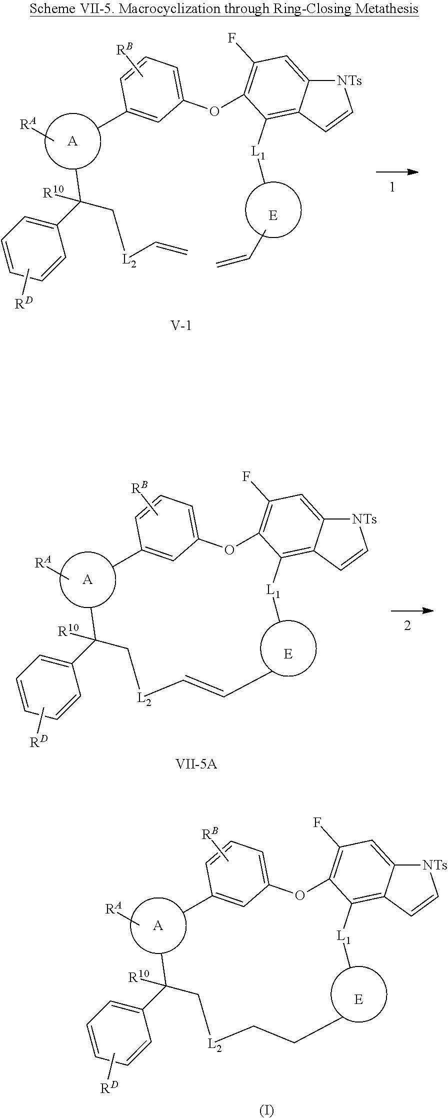

As illustrated Scheme VII-5, the proper starting material with required alkenes is subjected to Hoveyda-Grubb's catalytic conditions (Step 1) to form an alkene. After hydrogenation, the macrocycle (I) is obtained.

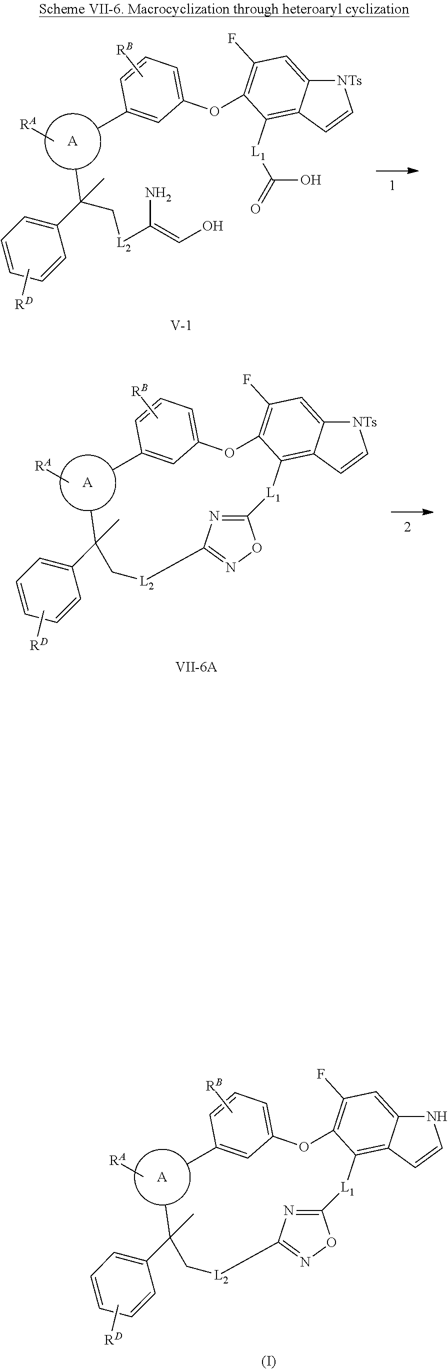

Macrocyclization is also achieved through direct heteroaryl cyclization as shown in Scheme VII-6. In step 1, an example of macrocyclization via an oxadiazole formation is illustrated. After removal of the protecting group (step 2), the designated compound of formula (I) is obtained.

### Analytical Methods:

Analytical Procedures ¹H NMR spectra were recorded with Bruker AC 400 MHz apparatus. Chemical shifts (δ) are quoted in parts per million (ppm) and coupling constants (J) in hertz (Hz).

The following liquid chromatography-Mass Spectrum (LC-MS) methods were used.

### LC-MS Method 1:

Spectra were obtained with UPLC Acquity device of Waters for liquid chromatography part, coupling with mass spectrometer ZMD of Waters. This system was piloted by MassLynx v4.1 software. Detection was made in UV at 220 nm. Operational conditions for liquid chromatography part are the following: Column: Assentis Express C18 50×2.1 mm, 2.7p Supelco Eluent: Way A: Water+0.02% trifluoroacetic acid; Way B: acetonitrile+0.014% trifluoroacetic acid; Gradient: T=0 minutes: 2% B; T=1 minutes: 98% B; T=1.3 minutes: 98% B, T=1.33 minutes: 2% B, T=1.5 minutes following injection; Flow: 1 mL/minutes; Temperature: 55° C. SQD: ESI+ 30V UV: 220 nm Injection: 0.2 μl.

### LC-MS Method 2:

Mobile Phase: A: water (0.01% trifluoroacetic acid), B: Acetonitrile (0.01% trifluoroacetic acid); Gradient: 5% B for 0.2 minutes, increased to 95% B within 1.3 minutes, 95% B for 1.5 minutes, back to 5% within 0.01 minutes; Flow Rate: 2 mL/minute; Column: Sunfire, 50×4.6 mm, 3.5 μm; Column Temperature: 50° C.

### LC-MS Method 3:

Mobile Phase: A: water (0.01% trifluoroacetic acid), B: Acetonitrile (0.01% trifluoroacetic acid); Column: Sunfire C18 4.6×50 mm, 3.5 μm; Elution program: Gradient from 5 to 95% of B in 1.4 minutes at 2.0 mL/minute; Temperature: 50° C.

### LC-MS Method 4:

Mobile Phase: A: water (0.01% trifluoroacetic acid) B: Acetonitrile (0.01% trifluoroacetic acid); Gradient: 5% B for 0.2 minutes, increase to 95% B within 1.3 minutes, 95% B for 1.5 minutes, back to 5% B within 0.01 minutes; Flow Rate: 1.8 mL/minutes; Column: Sunfire, 50*4.6 mm, 3.5 μm; Column Temperature: 50° C.

### LC-MS Method 5:

LC-Mass Method: Mobile Phase: A: water (0.1% formic acid) B: Acetonitrile (0.1% formic acid); Gradient: 5% B increase to 95% B within 1.3 minutes, 95% B for 1.5 minutes, back to 5% B within 0.01 min. Flow Rate: 2 mL/minute; Column: Sunfire C18, 4.6*50 mm, 3.5 μm.

### LC-MS Method 6:

Mobile Phase: A: water (2.5 mM trifluoroacetic acid); B: Acetonitrile (2.5 mM trifluoroacetic acid); Gradient: B=10%-95% in 1.0 min; Flow Rate: 1.5 mL/minute; Column: Xbridge-Cis, 4.6*30 mm, 2.5 μm.

### LC-MS Method 7:

A: water (10 mM ammonium bicarbonate) B: acetonitrile; Gradient: 5% B increase to 95% B within 1.5 minutes, 95% B for 1.5 minutes, back to 5% B within 0.01 minutes. Flow Rate: 1.8 mL/minute; Column: XBridge, 3.5 μm, 50*4.6 mm Oven Temperature: 50° C.

### LC-Mass Method 8:

Mobile phase: water (10 mM ammonium bicarbonate) (A)/acetonitrile (B); Gradient: B=5% B increase to 95% B within 1.4 minutes, 95% B for 1.6 minutes, back to 5% B within 0.01 minute; Flow rate: 1.8 mL/minute; Column: Xbridge-C18, 50×4.6 mm, 3.5 μm. Column Temperature: 50° C.

### LC-Mass Method 9:

Mobile Phase: A: water (0.01% trifluoroacetic acid); B: acetonitrile (0.01% trifluoroacetic acid) Gradient: 5% B for 0.2 minutes, increase to 95% B within 1.5 minutes, 95% B for 1.5 minutes, back to 5% B within 0.01 minutes; Flow Rate: 2 mL/minute; Column: Sunfire, 50*4.6 mm, 3.5 μm; Column Temperature: 50° C.

### LC-Mass Method 10:

Mobile Phase: A: water (0.01% trifluoroacetic acid); B: acetonitrile (0.01% trifluoroacetic acid) Gradient: 5% B for 0.2 minutes, increase to 95% B within 1.5 minutes, 95% B for 3.0 minutes, back to 5% B within 0.01 minutes; Flow Rate: 2 mL/minute; Column: Sunfire, 50*4.6 mm, 3.5 μm; Column Temperature: 50° C.

### LC-Mass Method 11:

Mobile Phase: A: water (0.01% trifluoroacetic acid) B: acetonitrile (0.01% trifluoroacetic acid); Gradient: 5% increase to 95% B within 2.5 minutes, 95% B for 2.5 minutes. Flow Rate: 2.0 mL/minutes; Column: Sunfire C18, 4.6*50 mm, 3.5 μm; Column Temperature: 45° C.; Detection: UV (214 nm, 4 nm) and MS (ESI, Positive mode, 110 to 1500 amu).

### LC-Mass Method 12:

Mobile Phase: A: water (10 mM ammonium bicarbonate) B: acetonitrile; Gradient: 5% B increase to 95% B within 1.2 minutes, 95% B for 1.5 minutes, back to 5% B within 0.01 minutes. Flow Rate: 1.8 mL/minute; Column: XBridge, 3.5 μm, 50*4.6 mm; Column Temperature: 50° C.

### LC-Mass Method 13:

Mobile Phase: A: water (10 mM ammonium bicarbonate) B: acetonitrile; Gradient: 10% B increase to 95% B within 1.5 minutes. Flow Rate: 1.8 mL/minute; Column: XBridge C18, 3.5 μm, 50*4.6 mm; Column Temperature: 50° C. Detection: UV (214, 4 nm) and MS (ESI, Pos mode, 132 to 1500 amu).

### LC-Mass Method 14:

Mobile Phase: A: water (10 mM ammonium bicarbonate) B: acetonitrile; Gradient: 5% B for 0.2 minutes, increase to 95% B within 1.3 minutes, 95% B for 1.5 minutes, back to 5% B within 0.01 minutes. Flow Rate: 2 mL/minute; Column: Sunfire 3.5 μm, 50*4.6 mm; Column Temperature: 50° C.

### LC-Mass Method 15:

Mobile Phase: A: water (0.01% trifluoroacetic acid) B: acetonitrile (0.01% trifluoroacetic acid). Gradient: 5%-95% B in 1.5 minutes, Flow Rate: 1.5 mL/minute; Column: KINETEX C18 5 μm, 3.0*30 mm

### LC-Mass Method 16:

Column: SunFire C18 (4.6×50 mm, 3.5 μm); Mobile phase: A: water (0.01% trifluoroacetic acid) B: acetonitrile (0.01% trifluoroacetic acid); Elution program: Gradient from 5 to 95% of B in 1.2 minutes at 2.0 mL/minute; Column Temperature: 50° C.; Detection: UV (214, 4 nm) and MS (ESI, Pos mode, 132 to 1500 amu).

### LC-Mass Method 17:

Mobile phase: A: water (0.01% trifluoroacetic acid) B: acetonitrile (0.01% trifluoroacetic acid) Gradient: 5% B for 0.2 minutes; Increase to 95% B within 3 minutes, 95% B for 2 minutes; Back to 5% B within 0.01 minutes; Flow Rate: 1.8 mL/minutes; Column: Sunfire, 50*4.6 mm, 3.5 μm; Column Temperature: 50° C.

### LC-Mass Method 18:

Mobile phase: A=10 mM trifluoroacetic acid/water, B=acetonitrile; Gradient: B=5%-95% in 1.5 minutes; Flow rate: 2.0 mL/minute; Column: Xbridge-C18, 50×4.6 mm, 3.5 μm.

### LC-Mass Method 19:

Mobile phase: A: water (0.01% trifluoroacetic acid) B: acetonitrile (0.01% trifluoroacetic acid); Elution program: Gradient from 5 to 95% of B in 2.5 minutes at 2 mL/minute Temperature: 50° C.

### LC-Mass Method 20:

Mobile Phase: A: water (0.01% trifluoroacetic acid) B: acetonitrile (0.01% trifluoroacetic acid); Gradient: 5%-95% B in 1.5 minutes. Flow Rate: 2 mL/min; Column: SunShell C18, 2.6 μm, 4.6*30 mm; Oven Temperature: 50° C.

### LC-Mass Method 21:

Mobile Phase: A: water (0.01% trifluoroacetic acid) B: acetonitrile (0.01% trifluoroacetic acid); Gradient: 5% B increase to 95% B within 1.3 minutes, 95% B for 0.7 minutes; Flow Rate: 2.5 mL/minute; Column: SunShell C18, 30*4.6 mm, 2.6 μm; Column Temperature: 40° C.

### LC-Mass Method 22:

Mobile Phase: A: water (0.1% formic acid) B: acetonitrile (0.1% formic acid); Gradient: 10% B increase to 90% B within 1.3 minutes, 90% B for 1.5 minutes, back to 5% within 0.01 minutes; Flow Rate: 1.8 mL/minute; Column: Sunfire C18, 50*4.6 mm, 3.5 μm; Column Temperature: 50° C.

### LC-Mass Method 23:

Mobile Phase: A: water (10 mM ammonium bicarbonate) B: acetonitrile; Gradient: 5% B to 95% B within 1.3 minutes; Flow Rate: 1.8 mL/minute; Column: XBridge C18 (4.6×50 mm, 3.5 μm)

### LC-Mass Method 24:

Mobile Phase: A: water (0.01% trifluoroacetic acid) B: acetonitrile (0.01% trifluoroacetic acid); Gradient: 5% B increase to 95% B within 1.2 minutes, 95% B for 0.8 minutes; Flow Rate: 1.8 mL/minute; Column: Zorbox SB—C18, 30*4.6 mm, 1.8 μm; Column Temperature: 40° C.

### LC-Mass Method 25:

Mobile Phase: A: water (10 mM ammonium bicarbonate) B: acetonitrile; Gradient: 5% B to 95% B within 1.5 minutes; Flow Rate: 2.0 mL/minute; Column: XBridge C18 (4.6×50 mm, 3.5 μm)

### LC-Mass Method 26:

Mobile Phase: A: water (0.01% trifluoroacetic acid), B: acetonitrile (0.01% trifluoroacetic acid); Gradient: 5% B increased to 95% B within 1.3 minutes, 95% B for 1.7 minutes, back to 5% B within 0.01 minutes; Flow Rate: 2 mL/minutes; Column: Sunfire, 50×4.6 mm, 3.5 μm; Column Temperature: 50° C.; Detection: UV (214.4 nm) and MS (ESI, Positive mode, 110 to 1000 amu).

### LC-Mass Method 27:

Mobile Phase: A: water (0.1% formic acid) B: Acetonitrile (0.1% formic acid) Gradient: 5% B increase to 95% B within 1.3 minutes, 95% B for 1.5 minutes, back to 5% B within 0.01 minutes; Flow Rate: 1.8 mL/minute; Column: XBridge C18, 4.6*50 mm, 3.5 μm.

### LC-Mass Method 28:

Mobile Phase: A: water (0.01% trifluoroacetic acid) B: Acetonitrile (0.01% trifluoroacetic acid); Gradient: 5% increase to 95% B within 1.5 minutes, 95% B for 1.7 minutes; Flow Rate: 2.0 mL/minute; Column: Sunfire C18, 4.6*50 mm, 3.5 μm.

### LC-Mass Method 29:

Mobile Phase: A: water (0.01% trifluoroacetic acid) B: Acetonitrile (0.01% trifluoroacetic acid); Gradient: 5% increase to 95% B within 1.4 minutes, 95% B for 1.4 minutes; back to 5% B within 0.1 minutes; Flow Rate: 2.0 mL/minute; Column: Sunfire C18, 4.6*50 mm, 3.5 μm; Column Temperature: 50° C.

### LC-Mass Method 30:

Mobile Phase: A: water (0.01% trifluoroacetic acid) B: Acetonitrile (0.01% trifluoroacetic acid); Gradient: 5% increase to 95% B within 1.2 minutes, 95% B for 1.8 minutes; back to 5% B within 0.01 minutes; Flow Rate: 2.0 mL/minute; Column: Sunfire C18, 4.6*50 mm, 3.5 μm; Column Temperature: 40° C.

### LC-Mass Method 31:

Mobile Phase: A: water (0.01% trifluoroacetic acid) B: acetonitrile (0.01% trifluoroacetic acid); Gradient: 5% B increase to 95% B within 1.3 minutes, 95% B for 0.7 minutes; Flow Rate: 1.8 mL/minute; Column: Chromolith Fast gradient RP-18e 50 mm*3 mm; Column Temperature: 40° C.; Detection: UV(214 nm, 4 nm) and MS (ESI, POS Mode, 110-1300 amu).

### LC-Mass Method 32:

Mobile phase: A: water (0.01% trifluoroacetic acid) B: acetonitrile (0.01% trifluoroacetic acid). Gradient: 5% B increase to 95% B within 1.3 minutes; 95% B for 1.2 minutes. Flow Rate: 2.2 mL/minutes; Column: Chromolith Fast Gradient RP-18e 3*50 mm. Column Temperature: 40° C.

### LC-Mass Method 33:

Mobile Phase: A: water (0.01% trifluoroacetic acid) B: Acetonitrile (0.01% trifluoroacetic acid); Gradient: 5% increase to 95% B within 1.3 minutes, 95% B for 1.7 minutes; Flow Rate: 1.6 mL/minute; Column: Agilent Poroshell, 30*3.0 mm, 2.7 μm; Column Temperature: 50° C. Detection: UV(214, 4 nm) and MS (ESI, Positive mode, 110 to 1000 amu).

### LC-Mass Method 34:

Mobile Phase: A: water (0.0% trifluoroacetic acid) B: Acetonitrile (0.01% trifluoroacetic acid); Gradient: 5% increase to 95% B within 1.5 minutes, 95% B for 1.7 minutes; Flow Rate: 2.0 mL/minute; Column: Sunfire C18, 4.6*50 mm, 3.5 μm; Column Temperature: 45° C.; Detection: UV(214 nm, 4 nm) and MS (ESI, Positive mode, 110 to 1000 amu).

### LC-Mass Method 35:

Mobile Phase: A: water (0.01% trifluoroacetic acid) B: Acetonitrile (0.01% trifluoroacetic acid) Gradient: 5% B increase to 95% B within 1.0 minute, 95% B for 1 minute; Flow Rate: 1.6 mL/minute; Column: Agilent Proshell 2.7 μm, 3.0 mm*30 mm Column Temperature: 50° C.; Detection: UV (214 nm, 4 nm) and MS (ESI, POS Mode, 110-1300 amu)

### LC-Mass Method 36:

Mobile Phase: A: water (0.01% trifluoroacetic acid) B: Acetonitrile (0.01% trifluoroacetic acid); Gradient: 5% increase to 95% B within 1.4 minutes, 95% B to 2.95 min, back to 5% B within 0.05 minutes (A total 3-minute method); Flow Rate: 2.0 mL/minute; Column: SunFire C18 3.5 μm 4.6*50 mm; Column Temperature: 40° C.; Detection: UV (214 nm, 4 nm), MS RANGE 115 to 1300

### LC-Mass Method 37:

Mobile Phase: A: water (0.01% trifluoroacetic acid) B: Acetonitrile (0.01% trifluoroacetic acid); Gradient: 5% B increase to 95% B within 2.5 minutes, 95% B for 2.5 minutes; Flow Rate: 1.8 mL/minute; Column: Agilent Poroshell 2.7 μm, 3.0 mm*30 mm; Column Temperature: 50° C.; Detection: UV (214 nm, 4 nm) and MS (ESI, Positive Mode, 110-1300 amu).

### LC-Mass Method 38:

Mobile Phase: A: water (0.01% trifluoroacetic acid) B: Acetonitrile (0.01% trifluoroacetic acid); Gradient: 20% B increase to 95% B within 2 minutes, 95% B for 3 minutes, back to 5% B within 0.01 minutes; Flow Rate: 2 mL/minute; Column: Agilent Poroshell, 30*3.0 mm, 2.7 μm; Column Temperature: 50° C. Detection: UV (214 nm, 4 nm) and MS (ESI, Positive Mode, 110-1300 amu).

### Preparation of Intermediates

### Intermediates 1: 2-(3-Bromophenyl)-2-Methyloct-7-Ynoic Acid

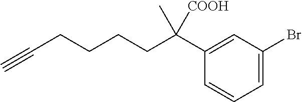 [SMILES: C#CCCCCC(C)(C(=O)O)c1cccc(Br)c1]

To a stirred solution of 2-(3-bromophenyl)-2-methyloct-7-ynenitrile (Intermediate 1B, 2.4 g, 7.24 mmol) in ethanol (80 mL) was added potassium hydroxide (12.2 g, 217.2 mmol) in water (40 mL). The mixture was heated for 24 hours at 80° C. and concentrated. The residue was acidified with 4 M hydrochloric acid (60 mL) and extracted with ethyl acetate (2×100 mL). The combined organic phases were washed with water, brine, dried over sodium sulfate, and concentrated. The residue was purified by silica gel column chromatography (petroleum ether/ethyl acetate=1:4) to give the title compound (1.7 g, 76% 2 steps) as a yellow oil. MS: 309, 311 m/z [M+H]⁺.

The following intermediates were prepared utilizing the procedure described for Intermediate 1 and/or for Intermediate 1A to 1B.

| Inter. | | | |
|---|---|---|---|
| No. | Structure | Name | MS m/z [M + H]⁺ |
| 1-1 | 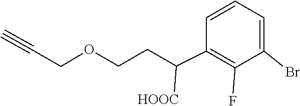 [SMILES: C#CCOCCC(C(=O)O)c1cccc(Br)c1F] | 2-(3-bromo-2- fluorophenyl)- 4-(prop-2- yn-1-yloxy) butanoic acid | 315, 317 |
| 1-2 | 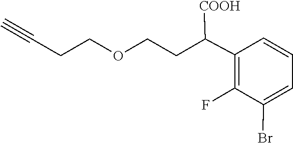 [SMILES: C#CCCOCCC(C(=O)O)c1cccc(Br)c1F] | 2-(3-bromo-2- fluorophenyl)- 4-(but-3- yn-1-yloxy) butanoic acid | 351, 353 [M + Na]⁺ |
| 1-3 | 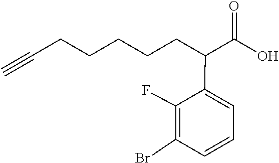 [SMILES: C#CCCCCCC(C(=O)O)c1cccc(Br)c1F] | 2-(3-bromo-2- fluorophenyl)non-8- ynoic acid | 327, 329 [M + H]⁺ |
| 1-4 | 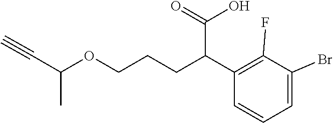 [SMILES: C#CC(C)OCCCC(C(=O)O)c1cccc(Br)c1F] | 2-(3-bromo-2- fluorophenyl)- 5-(but-3- yn-2-yloxy) pentanoic acid | MS: 343, 345[M + H]+; ¹H NMR (400 MHz, CDCl₃) δ 7.51-7.43 (m, 1H), 7.34- 7.27 (m, 1H), 7.04-6.99 (m, 1H), 4.17-4.06 (m, 1H), 4.00 (t, J = 7.6 Hz, 1H), 3.77-3.67 (m, 1H), 3.42-3.31 (m, 1H), 2.45-2.36 (m, 1H), 2.25-2.12 (m, 1H), 1.94-1.81 (m, 1H), 1.67-1.48 (m, 2H), 1.42 (d, J = 6.4 Hz, 3H) ppm. |
| 1-5 | 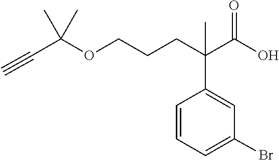 [SMILES: C#CC(C)(C)OCCCC(C)(C(=O)O)c1cccc(Br)c1] | 2-(3-bromophenyl)- 2-methyl-5-((2- methylbut- 3-yn-2-yl)oxy) pentanoic acid | 269, 271 [M − C5H8O]⁺ |

### Intermediates 1A: 2-(3-Bromophenyl)oct-7-ynenitrile

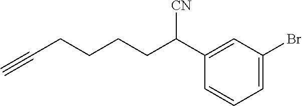 [SMILES: C#CCCCCC(C#N)c1cccc(Br)c1]

To a stirred and cooled (0° C.) solution of 2-(3-bromophenyl)acetonitrile (4.7 g, 24.02 mmol) in dimethylformamide (40 mL) was added sodium hydride (961 mg, 24.02 mmol, 60% dispersion in mineral oil). The mixture was stirred for 5 minutes, treated with 6-chlorohex-1-yne (1.4 g, 12.01 mmol) at 0° C., then warmed to room temperature and stirred for additional 2 hours. The solution was partitioned between water (50 mL) and ethyl acetate (50 mL). The separated organic phase, combined with one additional ethyl acetate extract (50 mL), was washed with water, brine, dried over sodium sulfate, and concentrated. The residue was purified by silica gel column chromatography (petroleum ether/ethyl acetate=20:1) to give the title compound (2.0 g, 61%) as a yellow oil. MS: 276, 278 m/z [M+H]⁺.

### Intermediates 1B: 2-(3-Bromophenyl)-2-methyloct-7-ynenitrile

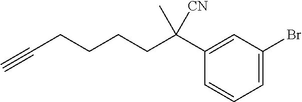 [SMILES: C#CCCCCC(C)(C#N)c1cccc(Br)c1]

To a stirred and cooled (0° C.) solution of 2-(3-bromophenyl)oct-7-ynenitrile (Intermediate 1A, 2.0 g, 7.24 mmol) in dimethylformamide (20 mL) was added sodium hydride (435 mg, 10.86 mmol). The mixture was stirred for 10 minutes, then treated with iodomethane (1.5 g, 10.86 mmol) at 0° C. The solution was warmed to room temperature and stirred for additional 2 hours and partitioned between water (50 mL) and ethyl acetate (100 mL). The separated organic layer was washed with water, brine, dried over sodium sulfate, and concentrated to give the title compound (2.4 g, crude) as yellow oil. MS: 290, 292 m/z [M+H]⁺.

### Intermediates 2: 1-Bromo-3-(3-bromophenyl)-3-methylnon-8-yn-2-one

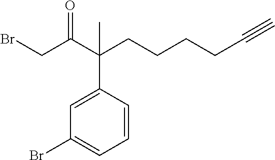 [SMILES: C#CCCCCC(C)(C(=O)CBr)c1cccc(Br)c1]

To a stirred and cooled (0° C.) solution of 2-(3-bromophenyl)-2-methyloct-7-ynoic acid (Intermediate 1, 800 mg, 2.59 mmol) in dichloromethane (20 mL) was added oxalyl chloride (987 mg, 7.77 mmol). The mixture was stirred at 0° C. for 1 hour and concentrated. The residue was dissolved in acetonitrile (10 mL) and treated with trimethylsilyl diazomethane (10.4 mL, 10.36 mmol) at 0° C. The solution was stirred for 18 hours at room temperature, then cooled to 0° C., treated with hydrogen bromide in acetic acid (45% in acetic acid, 1.9 g, 10.36 mmol) and stirred for an additional 1 hour. The mixture was diluted with ethyl acetate (2×40 mL), washed with water, saturated sodium bicarbonate solution, dried over sodium sulfate, and concentrated to give the title compound (1.0 g, 100%) as yellow oil. MS: 387 m/z [M+H]⁺.

The following intermediates were prepared utilizing the procedure described for Intermediate 2.

| Inter.No. | Structure | Name | MS m/z [M + H]⁺/¹H NMR |
|---|---|---|---|
| 2-1 | 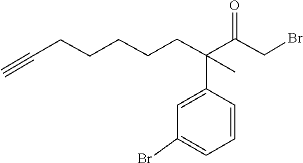 [SMILES: C#CCCCCCC(C)(C(=O)CBr)c1cccc(Br)c1] | 1-bromo-3-(3-bromophenyl)-3- methyldec-9-yn-2-one | 401 |
| 2-2 | 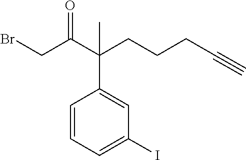 [SMILES: C#CCCCC(C)(C(=O)CBr)c1cccc(I)c1] | 1-bromo-3-(3-iodophenyl)-3- methyloct-7-yn-2-one | 419, 421 |
| 2-3 | 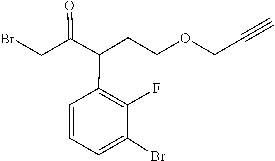 [SMILES: C#CCOCCC(C(=O)CBr)c1cccc(Br)c1F] | 1-bromo-3-(3-bromo-2- fluorophenyl)-5-(prop-2-yn-1- yloxy)pentan-2-one | 393 |
| 2-4 | 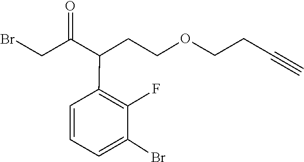 [SMILES: C#CCCOCCC(C(=O)CBr)c1cccc(Br)c1F] | 1-bromo-3-(3-bromo-2- fluorophenyl)-5-(but-3-yn-1- yloxy)pentan-2-one | 429 [M + Na]⁺ |
| 2-5 | 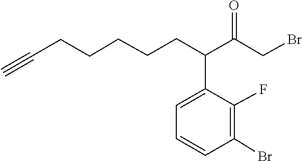 [SMILES: C#CCCCCCC(C(=O)CBr)c1cccc(Br)c1F] | 1-bromo-3-(3-bromo-2- fluorophenyl)dec-9-yn-2-one | 405 |
| 2-6 | 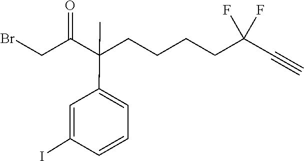 [SMILES: C#CC(F)(F)CCCCC(C)(C(=O)CBr)c1cccc(I)c1] | 1-bromo-8,8-difluoro-3-(3- iodophenyl)-3-methyldec-9-yn-2- one | 505, 507 [M + Na]⁺ |
| 2-7 | 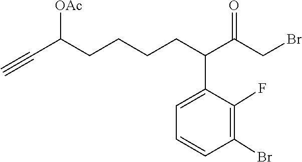 [SMILES: C#CC(CCCCC(C(=O)CBr)c1cccc(Br)c1F)OC(C)=O] | 10-bromo-8-(3-bromo-2- fluorophenyl)-9-oxodec-1-yn-3-yl acetate | 485 [M + Na]⁺ |
| 2-8 | 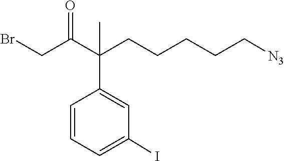 [SMILES: CC(CCCCCN=[N+]=[N-])(C(=O)CBr)c1cccc(I)c1] | 8-azido-1-bromo-3-(3-iodophenyl)- 3-methyloctan-2-one | MS m/z 486, 488 [M + Na]⁺. ¹H NMR (400 MHz, CDCl₃) δ 7.67-7.65 (m, 1H), 7.59-7.58 (m, 1H), 7.18-7.10 (m, 2H), 3.88- 3.76 (m, 2H), 3.26-3.22 (m, 2H), 1.97-1.89 (m, 2H), 1.59-1.53 (m, 5H), 1.41-1.36 (m, 2H), 1.15- 1.05 (m, 2H) ppm. |
| 2-9 | 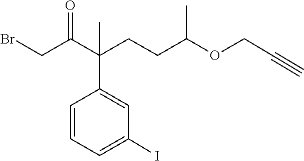 [SMILES: C#CCOC(C)CCC(C)(C(=O)CBr)c1cccc(I)c1] | 1-bromo-3-(3-iodophenyl)-3- methyl-6-(prop-2-yn-1- yloxy)heptan-2-one | 485, 487 [M + Na]⁺ |
| 2-10 | 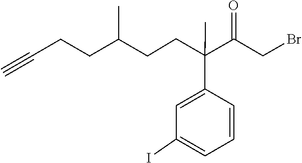 [SMILES: C#CCCC(C)CCC(C)(C(=O)CBr)c1cccc(I)c1] | 1-bromo-3-(3-iodophenyl)-3,6- dimethyldec-9-yn-2-one | 461, 463 |
| 2-11 | 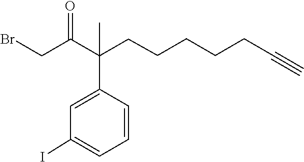 [SMILES: C#CCCCCCC(C)(C(=O)CBr)c1cccc(I)c1] | 1-bromo-3-(3-iodophenyl)-3- methyldec-9-yn-2-one | 447, 449 |
| 2-12 | 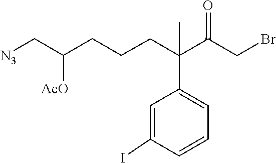 [SMILES: CC(=O)OC(CCCC(C)(C(=O)CBr)c1cccc(I)c1)CN=[N+]=[N-]] | 1-azido-8-bromo-6-(3-iodophenyl)- 6-methyl-7-oxooctan-2-yl acetate | 522, 524 |
| 2-13 | 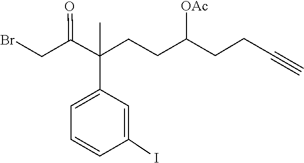 [SMILES: C#CCCC(CCC(C)(C(=O)CBr)c1cccc(I)c1)OC(C)=O] | 10-bromo-8-(3-iodophenyl)-8- methyl-9-oxodec-1-yn-5-yl acetate | 527, 529 [M + Na]⁺ |
| 2-14 | 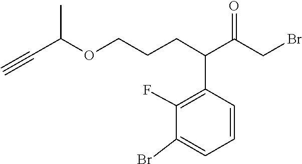 [SMILES: C#CC(C)OCCCC(C(=O)CBr)c1cccc(Br)c1F] | 1-bromo-3-(3-bromo-2- fluorophenyl)-6-(but-3-yn-2- yloxy)hexan-2-one | 421 |
| 2-15 | 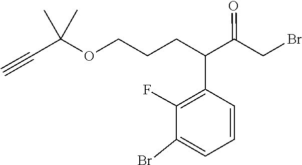 [SMILES: C#CC(C)(C)OCCCC(C(=O)CBr)c1cccc(Br)c1F] | 1-bromo-3-(3-bromo-2- fluorophenyl)-6-((2-methylbut-3-yn- 2-yl)oxy)hexan-2-one | 457 [M + Na]⁺ |
| 2-16 | 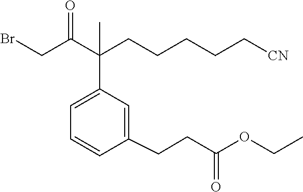 [SMILES: CCOC(=O)CCc1cccc(C(C)(CCCCCC#N)C(=O)CBr)c1] | ethyl 3-(3-(1-bromo-8-cyano-3- methyl-2-oxooctan-3- yl)phenyl)propanoate | 422, 424 |
| 2-17 | 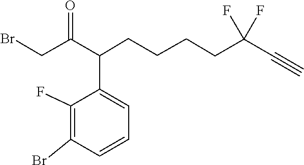 [SMILES: C#CC(F)(F)CCCCC(C(=O)CBr)c1cccc(Br)c1F] | 1-bromo-3-(3-bromo-2- fluorophenyl)-8,8-difluorodec-9-yn- 2-one | 463 [M + Na]⁺ |
| 2-18 | 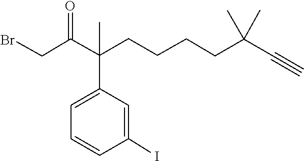 [SMILES: C#CC(C)(C)CCCCC(C)(C(=O)CBr)c1cccc(I)c1] | 1-bromo-3-(3-iodophenyl)-3,8,8- trimethyldec-9-yn-2-one | MS: 497, 499 m/z[M + Na]⁺; ¹H NMR (400 MHz, CDCl₃) δ 7.67-7.62 (m, 1H), 7.58 (t, J = 1.6 Hz, 1H), 7.15-7.10 (m, 2H), 3.85-3.78 (m, 2H), 2.05-1.91 (m, 3H), 1.52 (s, 3H), 1.48-1.41 (m, 2H), 1.39-1.32 (m, 2H), 1.17- 1.12 (m, 8H) ppm. |
| 2-19 | 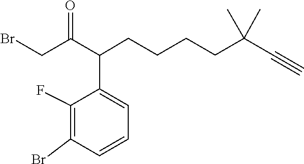 [SMILES: C#CC(C)(C)CCCCC(C(=O)CBr)c1cccc(Br)c1F] | 1-bromo-3-(3-bromo-2- fluorophenyl)-8,8-dimethyldec-9- yn-2-one | 455 [M + Na]⁺ |
| 2-20 | 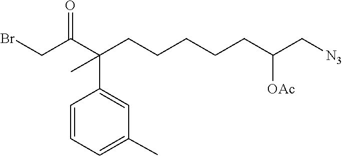 [SMILES: CC(=O)OC(CCCCCC(C)(C(=O)CBr)c1cccc(C)c1)CN=[N+]=[N-]] | 1-azido-10-bromo-8-(3-iodophenyl)- 8-methyl-9-oxodecan-2-yl acetate | 550, 552 |
| 2-21 | 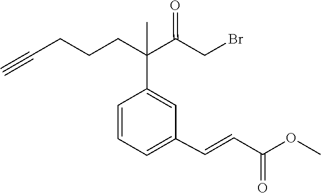 [SMILES: C#CCCCC(C)(C(=O)CBr)c1cccc(/C=C(\C)C(=O)OC)c1] | methyl (E)-3-(3-(1-bromo-3-methyl- 2-oxooct-7-yn-3-yl)phenyl)acrylate | 377, 379 |
| 2-22 | 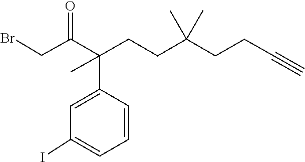 [SMILES: C#CCCC(C)(C)CCC(C)(C(=O)CBr)c1cccc(I)c1] | 1-bromo-3-(3-iodophenyl)-3,6,6- trimethyldec-9-yn-2-one | 497, 499 [M + Na]⁺ |
| 2-23 | 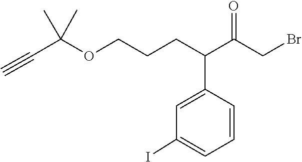 [SMILES: C#CC(C)(C)OCCCC(C(=O)CBr)c1cccc(I)c1] | 1-bromo-3-(3-iodophenyl)-6-((2- methylbut-3-yn-2-yl)oxy)hexan-2- one | 485, 487 [M + Na]⁺ |
| 2-24 | 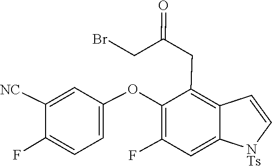 [SMILES: Cc1ccc(S(=O)(=O)n2ccc3c(CC(=O)CBr)c(Oc4ccc(F)c(C#N)c4)c(F)cc32)cc1] | 5-((4-(3-bromo-2-oxopropyl)-6- fluoro-1-tosyl-1H-indol-5-yl)oxy)-2- fluorobenzonitrile | 559, 561 |
| 2-25 | 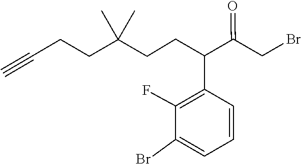 [SMILES: C#CCCC(C)(C)CCC(C(=O)CBr)c1cccc(Br)c1F] | 1-bromo-3-(3-bromo-2- fluorophenyl)-6,6-dimethyldec-9- yn-2-one | 455 [M + Na]⁺ |
| 2-26 | 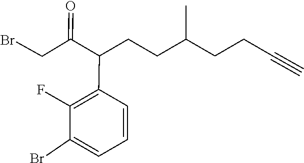 [SMILES: C#CCCC(C)CCC(C(=O)CBr)c1cccc(Br)c1F] | 1-bromo-3-(3-bromo-2- fluorophenyl)-6-methyldec-9-yn-2- one | 419 |
| 2-27 | 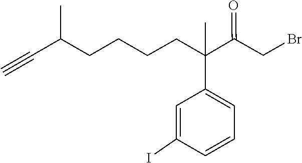 [SMILES: C#CC(C)CCCCC(C)(C(=O)CBr)c1cccc(I)c1] | 1-bromo-3-(3-iodophenyl)-3,8- dimethyldec-9-yn-2-one | 483, 485 [M + Na]⁺ |
| 2-28 | 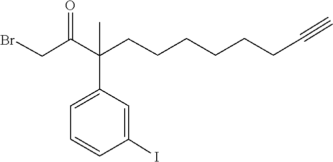 [SMILES: C#CCCCCCCC(C)(C(=O)CBr)c1cccc(I)c1] | 1-bromo-3-(3-iodophenyl)-3- methylundec-10-yn-2-one | 483, 485 [M + Na]⁺ |
| 2-29* | 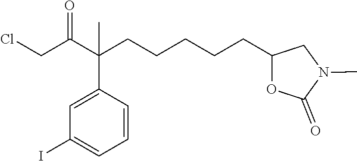 [SMILES: CN1CC(CCCCCC(C)(C(=O)CCl)c2cccc(I)c2)OC1=O] | 5-(8-chloro-6-(3-iodophenyl)-6- methyl-7-oxooctyl)-3- methyloxazolidin-2-one | 478 |
| 2-30 | 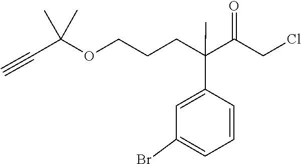 [SMILES: C#CC(C)(C)OCCCC(C)(C(=O)CCl)c1cccc(Br)c1] | 3-(3-bromophenyl)-1-chloro-3- methyl-6-((2-methylbut-3-yn-2- yl)oxy)hexan-2-one | 385, 387 |
| 2-31 | 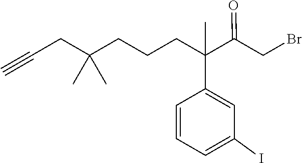 [SMILES: C#CCC(C)(C)CCCC(C)(C(=O)CBr)c1cccc(I)c1] | 1-bromo-3-(3-iodophenyl)-3,7,7- trimethyldec-9-yn-2-one | 497, 499 [M + Na]⁺ |
| 2-32 | 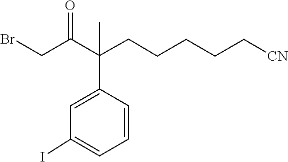 [SMILES: CC(CCCCCC#N)(C(=O)CBr)c1cccc(I)c1] | 9-bromo-7-(3-iodophenyl)-7- methyl-8-oxononanenitrile | 448, 450 |
| 2-33 | 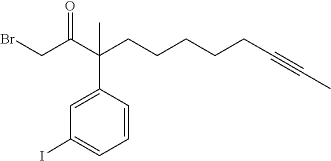 [SMILES: CC#CCCCCCC(C)(C(=O)CBr)c1cccc(I)c1] | 1-bromo-3-(3-iodophenyl)-3- methylundec-9-yn-2-one | 461, 463 |
| 2-34 | 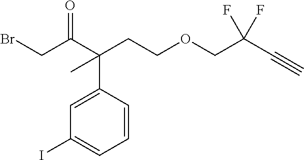 [SMILES: C#CC(F)(F)COCCC(C)(C(=O)CBr)c1cccc(I)c1] | 1-bromo-5-((2,2-difluorobut-3-yn-1- yl)oxy)-3-(3-iodophenyl)-3- methylpentan-2-one | 507, 509 [M + Na]⁺ |
| 2-35 | 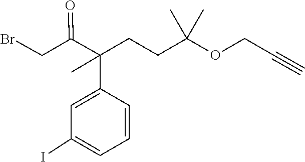 [SMILES: C#CCOC(C)(C)CCC(C)(C(=O)CBr)c1cccc(I)c1] | 1-bromo-3-(3-iodophenyl)-3,6- dimethyl-6-(prop-2-yn-1- yloxy)heptan-2-one | ¹H NMR (400 MHz, CDCl₃) δ 7.68-7.55 (m, 2H), 7.20-7.00 (m, 2H), 4.04-3.92 (m, 2H), 3.85 (t, J = 8.4 Hz, 2H), 2.41-2.31 (m, 1H), 2.14-1.90 (m, 2H), 1.54- 1.50 (m, 3H), 1.42-1.21 (m, 2H), 1.20 (s, 3H), 1.18 (s, 3H) ppm. |
| 2-36 | 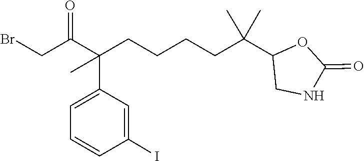 [SMILES: CC(CCCCC(C)(C)C1CNC(=O)O1)(C(=O)CBr)c1cccc(I)c1] | 5-(9-bromo-7-(3-iodophenyl)-2,7- dimethyl-8-oxononan-2- yl)oxazolidin-2-one | 536, 538 |
| 2-37 | 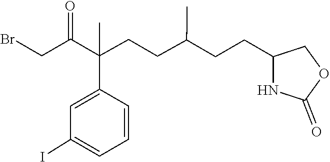 [SMILES: CC(CCC1COC(=O)N1)CCC(C)(C(=O)CBr)c1cccc(I)c1] | 4-(8-bromo-6-(3-iodophenyl)-3,6- dimethyl-7-oxooctyl)oxazolidin-2- one | 522, 524 |
| 2-38 | 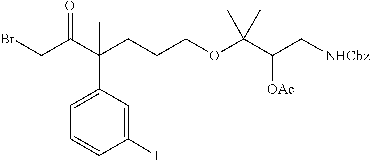 [SMILES: CC(=O)OC(CNC(=O)OCc1ccccc1)C(C)(C)OCCCC(C)(C(=O)CBr)c1cccc(I)c1] | 1-(((benzyloxy)carbonyl)amino)-3- ((6-bromo-4-(3-iodophenyl)-4- methyl-5-oxohexyl)oxy)-3- methylbutan-2-yl acetate | 710, 712 [M + Na]⁺ |
| 2-39 | 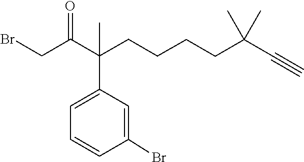 [SMILES: C#CC(C)(C)CCCCC(C)(C(=O)CBr)c1cccc(Br)c1] | 1-bromo-3-(3-bromophenyl)-3,8,8- trimethyldec-9-yn-2-one | ¹H NMR (400 MHz, CDCl₃) δ 7.46-7.42 (m, 1H), 7.41-7.39 (m, 1H), 7.25-7.20 (m, 1H), 7.14- 7.10 (m, 1H), 3.84-3.80 (m, 2H), 2.06-1.88 (m, 3H), 1.54 (s, 3H), 1.48-1.41 (m, 2H), 1.39-1.32 (m, 2H), 1.28-1.24 (m, 2H), 1.18 (s, 6H) ppm. |
| 2-40 | 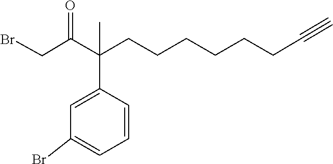 [SMILES: C#CCCCCCCC(C)(C(=O)CBr)c1cccc(Br)c1] | 1-bromo-3-(3-bromophenyl)-3- methylundec-10-yn-2-one | ¹H NMR (400 MHz, CDCl₃) δ 7.48-7.37 (m, 2H), 7.23 (d, J = 7.9 Hz, 1H), 7.14-7.10 (m, 1H), 3.91-3.70 (m, 2H), 2.18-2.14 (m, 2H), 1.93-1.88 (m, 2H), 1.55- 1.44 (m, 5H), 1.43-1.22 (m, 5H), 1.19-1.00 (m, 2H) ppm. |
| 2-41 | 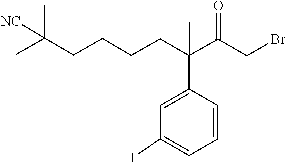 [SMILES: CC(C)(C#N)CCCCC(C)(C(=O)CBr)c1cccc(I)c1] | 9-bromo-7-(3-iodophenyl)-2,2,7- trimethyl-8-oxononanenitrile | 498, 500 [M + Na]⁺ |
| 2-42 | 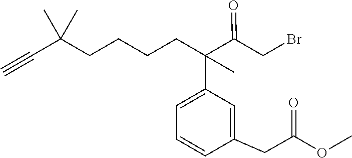 [SMILES: C#CC(C)(C)CCCCC(C)(C(=O)CBr)c1cccc(CC(=O)OC)c1] | methyl 2-(3-(1-bromo-3,8,8- trimethyl-2-oxodec-9-yn-3- yl)phenyl)acetate | 443, 445 [M + Na]⁺ |
| 2-43 | 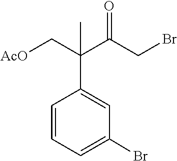 [SMILES: CC(=O)OCC(C)(C(=O)CBr)c1cccc(Br)c1] | 4-bromo-2-(3-bromophenyl)-2- methyl-3-oxobutyl acetate | ¹H NMR (400 MHz, CDCl₃) δ 7.52-7.46 (m, 1H), 7.41 (t, J = 1.7 Hz, 1H), 7.31-7.24 (m, 1H), 7.15 (d, J = 7.9 Hz, 1H), 4.56 (d, J = 11.3 Hz, 1H), 4.35 (d, J = 11.3 Hz, 1H), 3.93-3.90 (m, 2H), 2.02 (s, 3H), 1.66 (s, 3H) ppm. |
| 2-44 | 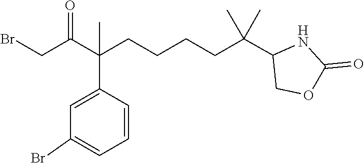 [SMILES: CC(CCCCC(C)(C)C1COC(=O)N1)(C(=O)CBr)c1cccc(Br)c1] | 4-(9-bromo-7-(3-bromophenyl)-2,7- dimethyl-8-oxononan-2- yl)oxazolidin-2-one | 490 |
| 2-45 | 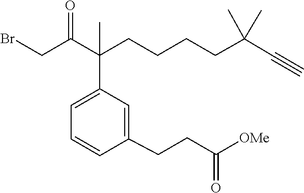 [SMILES: C#CC(C)(C)CCCCC(C)(C(=O)CBr)c1cccc(CCC(=O)OC)c1] | methyl 3-(3-(1-bromo-3,8,8- trimethyl-2-oxodec-9-yn-3- yl)phenyl)propanoate | MS: 435, 437 [M + H]⁺; ¹H NMR (400 MHz, CDCl₃) δ 7.29 (t, J = 7.6 Hz, 1H), 7.13 (d, J = 7.6 Hz, 1H), 7.06 (d, J = 7.6 Hz, 1H), 7.02 (s, 1H), 3.86-3.72 (m, 2H), 3.67 (s, 3H), 2.95 (t, J = 7.6 Hz, 2H), 2.62 (t, J = 8.0 Hz, 2H), 2.07-2.03 (m, 1H), 1.99-1.94 (m, 2H), 1.53 (s, 3H), 1.48-1.41 (m, 2H), 1.36-1.32 (m, 2H), 1.17 (s, 6H), 1.15-1.06 (m, 2H) ppm. |
| 2-46 | 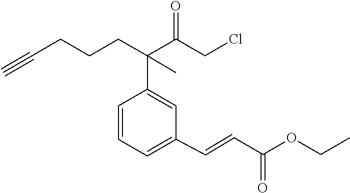 [SMILES: C#CCCCC(C)(C(=O)CCl)c1cccc(/C=C/C(=O)OCC)c1] | ethyl (E)-3-(3-(1-chloro-3-methyl-2- oxooct-7-yn-3-yl)phenyl)acrylate | 347 |
| 2-47 | 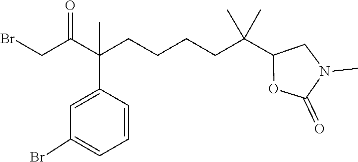 [SMILES: CN1CC(C(C)(C)CCCCC(C)(C(=O)CBr)c2cccc(Br)c2)OC1=O] | 5-(9-bromo-7-(3-bromophenyl)-2,7- dimethyl-8-oxononan-2-yl)-3- methyloxazolidin-2-one | 504 |
| 2-48 | 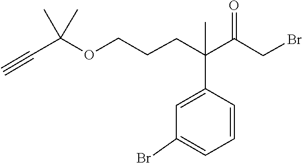 [SMILES: C#CC(C)(C)OCCCC(C)(C(=O)CBr)c1cccc(Br)c1] | 1-bromo-3-(3-bromophenyl)-3- methyl-6-((2-methylbut-3-yn-2- yl)oxy)hexan-2-one | 453 [M + Na]⁺ |
| 2-49 | 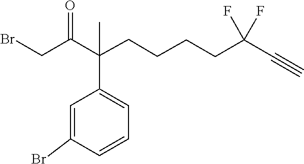 [SMILES: C#CC(F)(F)CCCCC(C)(C(=O)CBr)c1cccc(Br)c1] | 1-bromo-3-(3-bromophenyl)-8,8- difluoro-3-methyldec-9-yn-2-one | 459 [M + Na]⁺ |
| 2-50 | 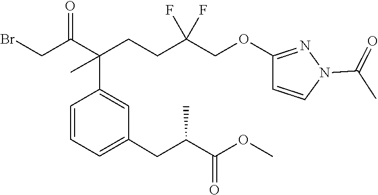 [SMILES: COC(=O)[C@@H](C)Cc1cccc(C(C)(CCC(F)(F)COc2ccn(C(C)=O)n2)C(=O)CBr)c1] | methyl (2S)-3-(3-(7-((1-acetyl-1H- pyrazol-3-yl)oxy)-1-bromo-6,6- difluoro-3-methyl-2-oxoheptan-3- yl)phenyl)-2-methylpropanoate | 543, 545 |
| 2-51 | 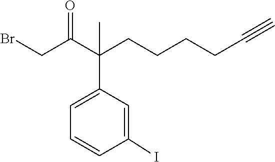 [SMILES: C#CCCCCC(C)(C(=O)CBr)c1cccc(I)c1] | 1-bromo-3-(3-iodophenyl)-3- methylnon-8-yn-2-one | 433, 435 |
| 2-52 | 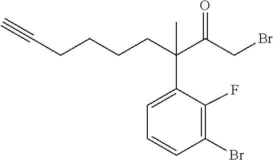 [SMILES: C#CCCCCC(C)(C(=O)CBr)c1cccc(Br)c1F] | 1-bromo-3-(3-bromo-2- fluorophenyl)-3-methylnon-8-yn-2- one | 405 |
| 2-53 | 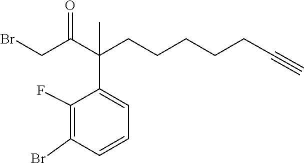 [SMILES: C#CCCCCCC(C)(C(=O)CBr)c1cccc(Br)c1F] | 1-bromo-3-(3-bromo-2- fluorophenyl)-3-methyldec-9-yn-2- one | 419 |
| 2-54 | 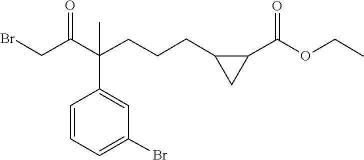 [SMILES: CCOC(=O)C1CC1CCCC(C)(C(=O)CBr)c1cccc(Br)c1] | ethyl 2-(6-bromo-4-(3- bromophenyl)-4-methyl-5- oxohexyl)cyclopropane-1- carboxylate | 483 [M + Na]⁺ |

*Replacing hydrogen bromide in acetic acid with hydrogen chloride in 1,4-dioxane to quench diazoketone will afford a chloro-ketone.

### Intermediate 3: 5-((4-Bromo-6-fluoro-1H-indol-5-yl)oxy)-2-fluorobenzonitrile

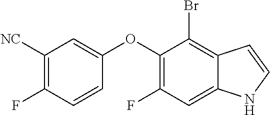 [SMILES: N#Cc1cc(Oc2c(F)cc3[nH]ccc3c2Br)ccc1F]

Step One: To a stirred solution of Intermediate 3B (124 g, 336 mmol) in N,N-dimethylformamide (1 L) was added N,N-dimethylformamide dimethyl acetal (178 mL, 159 g, 1.34 mol). Five identical reactions were executed in parallel. The six mixtures were each heated at 100° C. for six hours and then cooled to room temperature, combined, and poured into stirred ice water (20 L). After warming to near room temperature, the suspension was extracted with ethyl acetate (2×8 L). The combined organic layers were washed with water (1×10 L) and brine (1×10 L), dried over sodium sulfate and concentrated. The crude N,N-dimethyl enamine intermediate, which was used without purification in the second step, was afforded as a black oil (786 g, 92%). ¹H NMR (400 MHz CDCl₃) δ 7.38 (d, J=8.8 Hz, 1H), 7.19 (s, 1H), 7.10-7.12 (m, 2H), 6.44 (d, J=13.6 Hz, 1H), 4.96 (d, J=13.6 Hz, 1H), 2.80 (s, 6H) ppm.

Step Two: To a stirred solution of the crude enamine (100 g, 236 mmol) in a mixture of acetic acid (800 mL) and toluene (800 mL) was added silica gel (42.5 g). The suspension was warmed to 50° C. and treated with iron powder (132 g, 2.36 mol), portion-wise over 15 minutes. Following this addition, the mixture was heated at 100° C. for 12 hours and then cooled to room temperature and suction filtered through a bed of Celite. The filtering agent was rinsed with ethyl acetate (total, 5 L) and the combined filtrate was partitioned between water (10 L) and ethyl acetate (5 L). The organic layer was combined with a second extract (ethyl acetate, 1×5 L), washed with water (1×10 L) and brine (1×10 L), dried over sodium sulfate, and concentrated under reduced pressure to give a dark brown oil. The resulting dark brown oil was purified by automated flash chromatography (1 kg silica gel column, 1-20% ethyl acetate in petroleum ether) to afford the title compound as a white solid (66.7 g, 74% overall, two steps). ¹H NMR (400 MHz CDCl₃) δ 8.41 (s, 1H), 7.25-7.26 (m, 1H), 7.15-7.18 (m, 3H), 6.95-7.09 (m, 1H), 6.55 (t, J=2.8 Hz, 1H) ppm.

The following intermediates were prepared utilizing the procedure described for Intermediate 3 and/or for Intermediate 3A to 3B.

| Inter.No. | Structure | Name | MS m/z [M + H]⁺/¹H NMR |
|---|---|---|---|
| 3-1 | 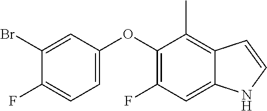 [SMILES: Cc1c(Oc2ccc(F)c(Br)c2)c(F)cc2[nH]ccc12] | 5-(3-bromo-4-fluorophenoxy)- 6-fluoro-4-methyl-1H-indole | (400 MHz, CDCl₃) δ 8.29 (s, 1H), 7.30- 7.23 (m, 1H), 7.08 (d, J = 10.0 Hz, 1H), 7.04-6.99 (m, 2H), 6.84-6.77 (m, 1H), 6.57 (s, 1H), 2.39 (s, 3H) ppm. |
| 3-2 | 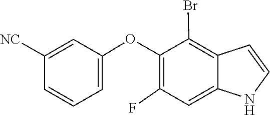 [SMILES: N#Cc1cccc(Oc2c(F)cc3[nH]ccc3c2Br)c1] | 3-((4-bromo-6-fluoro-1H- indol-5-yl)oxy)benzonitrile | 331, 333 |
| 3-3 | 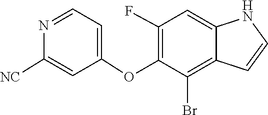 [SMILES: N#Cc1cc(Oc2c(F)cc3[nH]ccc3c2Br)ccn1] | 4-((4-bromo-6-fluoro-1H- indol-5-yl)oxy)picolinonitrile | 332, 334 |
| 3-4 | 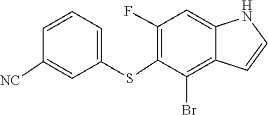 [SMILES: N#Cc1cccc(Sc2c(F)cc3[nH]ccc3c2Br)c1] | 3-((4-bromo-6-fluoro-1H- indol-5-yl)thio)benzonitrile | 347, 349 |
| 3-5 | 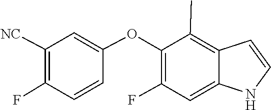 [SMILES: Cc1c(Oc2ccc(F)c(C#N)c2)c(F)cc2[nH]ccc12] | 2-fluoro-5-((6-fluoro-4- methyl-1H-indol-5- yl)oxy)benzonitrile | 285 m/z [M + H]⁺; (400 MHz, CDCl₃) δ 8.28 (s, 1H), 7.27 (s, 1H), 7.20-7.07 (m, 3H), 6.98 (dd, J = 4.8, 2.8 Hz, 1H), 6.58 (s, 1H), 2.39 (s, 3H) ppm. |

### Intermediate 3A: 3-Bromo-1,2-difluoro-4-methyl-5-nitrobenzene

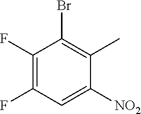

To a stirred solution of 1,2-difluoro-4-methyl-5-nitrobenzene (150 g, 866 mmol) in trifluoroacetic acid (800 mL) was added 1,3-dibromo-5,5-dimethylimidazolidine-2,4-dione (136 g, 476 mmol) and concentrated sulfuric acid (200 mL; over 3-4 minutes). Two additional bromination reactions, utilizing the same quantities of reactants and solvent, were run in parallel. After 10 hours at room temperature, the three reactions were combined and then slowly poured into a stirred slurry of crushed ice and water (5 L). When the ice had fully melted, the mixture was extracted with petroleum ether (2×4 L). The combined organic layers were washed with brine (1×5 L), dried over sodium sulfate and concentrated. The resulting oil was purified by automated flash chromatography (3 kg silica gel column, 100% petroleum ether) to afford the title compound as a yellow oil (417 g, 64%). ¹H NMR (400 MHz CDCl₃) δ 7.70-7.64 (m, 1H), 2.55 (s, 3H) ppm.

### Intermediate 3B: 5-(2-Bromo-6-fluoro-3-methyl-4-nitrophenoxy)-2-fluorobenzonitrile

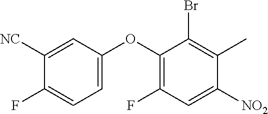 [SMILES: Cc1c([N+](=O)[O-])cc(F)c(Oc2ccc(F)c(C#N)c2)c1Br]

To a stirred solution of Intermediate 3A (207 g, 820 mmol) in N,N-dimethylformamide (1 L) was added 2-fluoro-5-hydroxybenzonitrile (118 g, 861 mmol) and potassium carbonate (227 g, 1.64 mol). A second identical reaction was run in parallel. Both mixtures were heated at 100° C. for one hour and then cooled to room temperature, combined, and poured into stirred ice water (7 L). After warming to room temperature, the resulting suspension was extracted with ethyl acetate (2×3 L). The combined organic layers were washed with water (1×5 L) and brine (1×3 L), dried over sodium sulfate and concentrated. The crude title compound, which was used without purification, was afforded as a yellow solid (585 g, 97%). ¹H NMR (400 MHz CDCl₃) δ 7.68 (d, J=9.2 Hz, 1H), 7.10-7.14 (m, 2H), 7.01-7.02 (m, 1H), 2.58 (s, 3H).

The following intermediates were prepared utilizing the procedures described for Intermediate 3B.

| Inter | | | MS |
|---|---|---|---|
| No. | Structure | Name | [M + H]⁺ |
| 3B-1 | 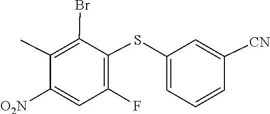 [SMILES: Cc1c([N+](=O)[O-])cc(F)c(Sc2cccc(C#N)c2)c1Br] | 3-((2-bromo-6-fluoro-3-methyl-4- nitrophenyl)thio)benzonitrile | 367, 369 |

### Intermediate 4: 5-((4-Bromo-6-fluoro-1-tosyl-1H-indol-5-yl)oxy)-2-fluorobenzonitrile

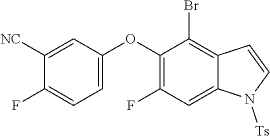 [SMILES: Cc1ccc(S(=O)(=O)n2ccc3c(Br)c(Oc4ccc(F)c(C#N)c4)c(F)cc32)cc1]

To a solution of 5-((4-bromo-6-fluoro-1H-indol-5-yl)oxy)-2-fluorobenzonitrile (Intermediate 3, 72 g, 264 mmol) in N,N-dimethylformamide (500 mL) was carefully added sodium hydride (12.7 g, 317 mmol, 60% purity) at room temperature. The reaction mixture was stirred for 30 minutes, 4-methylbenzenesulfonyl chloride (47.2 g, 247 mmol) was then added portion wise and continued stirring for 9.5 hours. The reaction was quenched with water (3 L) and extracted with ethyl acetate (2 L×2). The combined organic extracts were washed with brine (2 L), dried over sodium sulfate, filtered, and concentrated to afford the title compound as a white solid (144 g). ¹H NMR: (400 MHz CDCl₃) δ 7.88-7.84 (m, 1H), 7.79 (d, J=8.0 Hz, 2H), 7.67 (d, J=2.8 Hz, 1H), 7.31 (d, J=8.0 Hz, 2H), 7.16-7.12 (m, 2H), 6.98 (s, 1H), 6.72 (d, J=2.8 Hz, 1H), 2.39 (s, 3H) ppm.

The following intermediates were prepared utilizing the procedures described for Intermediate 4.

| Inter | | | |
|---|---|---|---|
| No. | Structure | Name | ¹H NMR |
| 4-1 | 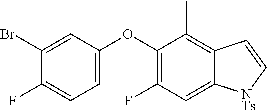 [SMILES: Cc1ccc(S(=O)(=O)n2ccc3c(C)c(Oc4ccc(F)c(Br)c4)c(F)cc32)cc1] | 5-(3-bromo-4- fluorophenoxy)- 6-fluoro-4- methyl-1-tosyl-1H- indole | (400 MHz, CDCl₃) 7.80-7.77 (m, 2H), 7.70-7.67 (m, 1H), 7.61-7.58 (m, 1H), 7.31- 7.27 (m, 2H), 6.96-6.03 (m, 2H), 6.73-6.33 (m, 1H), 6.68-6.64 (m, 1H), 2.39 (s, 3H), 2.30 (s, 3H) ppm. |
| 4-2 | 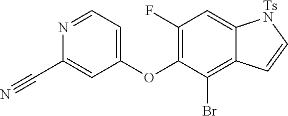 [SMILES: Cc1ccc(S(=O)(=O)n2ccc3c(Br)c(Oc4ccnc(C#N)c4)c(F)cc32)cc1] | 4-((4-bromo-6- fluoro-1-tosyl- 1H-indol-5- yl)oxy)picolinonitrile | 486, 488 |

### Intermediate 5: 2-Fluoro-5-((6-fluoro-4-(2-hydroxyethyl)-1-tosyl-1H-indol-5-yl)oxy)benzonitrile

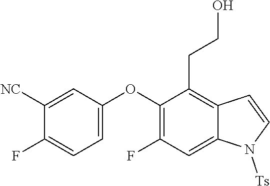 [SMILES: Cc1ccc(S(=O)(=O)n2ccc3c(CCO)c(Oc4ccc(F)c(C#N)c4)c(F)cc32)cc1]

To a stirred and cooled (0° C.) solution of 2-fluoro-5-((6-fluoro-4-(2-oxoethyl)-1-tosyl-1H-indol-5-yl)oxy)benzonitrile (Intermediate 5B, 3.2 g, 6.88 mmol in methanol (40 mL) was added sodium borohydride (781 mg, 20.64 mmol). The mixture was stirred at room temperature for 1 hour, quenched with water (20 mL) and concentrated to remove methanol. The aqueous residue was extracted with ethyl acetate (2×50 mL). The combined organic phases were washed with brine, dried over sodium sulfate, and concentrated. The residue was purified by silica gel column chromatography (petroleum ether/ethyl acetate=1:1) to give the title compound (3.0 g, 97%) as a white solid. MS: 469 m/z [M+H]⁺.

### Intermediate 5A: (E)-5-((4-(2-Ethoxyvinyl)-6-fluoro-1-tosyl-1H-indol-5-yl)oxy)-2-fluorobenzonitrile

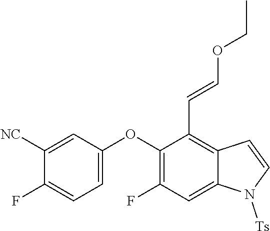 [SMILES: CCO/C=C/c1c(Oc2ccc(F)c(C#N)c2)c(F)cc2c1ccn2S(=O)(=O)c1ccc(C)cc1]

To a stirred and degassed solution of 5-((4-bromo-6-fluoro-1-tosyl-1H-indol-5-yl)oxy)-2-fluorobenzonitrile (Intermediate 4, 5.0 g, 19.93 mmol)) in dimethylformamide (50 mL) was added tributyl(2-ethoxyvinyl)stannane (4.3 g, 11.92 mmol), bis(triphenylphosphine) palladium (II) dichloride (695 mg, 0.99 mmol) and lithium chloride (1.3 g, 29.79 mmol) in a sealed tube. The mixture was heated at 100° C. for 4 hours. The reaction was cooled to room temperature, quenched with saturated potassium fluoride (50 mL), and extracted with ethyl acetate (3×50 mL). The combined organic phases were washed with water, brine, dried over sodium sulfate, and concentrated. The residue was purified by silica gel column chromatography (petroleum ether/ethyl acetate=4:1) to give the title compound (3.4 g, 66%) as yellow solid. MS: 495 m/z [M+H]⁺.

### Intermediate 5B: 2-Fluoro-5-((6-fluoro-4-(2-oxoethyl)-1-tosyl-1H-indol-5-yl)oxy)benzonitrile

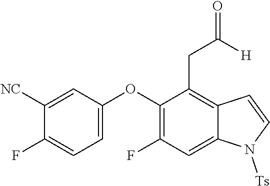 [SMILES: Cc1ccc(S(=O)(=O)n2ccc3c(CC=O)c(Oc4ccc(F)c(C#N)c4)c(F)cc32)cc1]

To a stirred solution of (E)-5-((4-(2-ethoxyvinyl)-6-fluoro-1-tosyl-1H-indol-5-yl)oxy)-2-fluorobenzonitrile (Intermediate 5A, 3.3 g, 6.67 mmol) in tetrahydrofuran (100 mL) was added 1 M hydrochloric acid (100 mL). The mixture was stirred at room temperature for 16 hours, then extracted with ethyl acetate (3×100 mL). The combined organic phases were washed with brine, dried over sodium sulfate, and concentrated to give the title compound (3.2 g, 100%) as a yellow oil. MS: 467 m/z [M+H]⁺.

### Intermediate 6: 5-((4-(2-Azidoethyl)-6-fluoro-1H-indol-5-yl)oxy)-2-fluorobenzimidamide

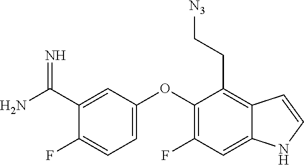 [SMILES: [N-]=[N+]=NCCc1c(Oc2ccc(F)c(C(=N)N)c2)c(F)cc2[nH]ccc12]

To a stirred and cooled (0° C.) solution of 5-((4-(2-azidoethyl)-6-fluoro-1-tosyl-1H-indol-5-yl)oxy)-2-fluorobenzonitrile (Intermediate 6B, 2.0 g, 4.05 mmol) in tetrahydrofuran (10 mL) was added lithium bis(trimethylsilyl)amide (40.5 mL, 40.5 mmol, 1 M in tetrahydrofuran). The mixture was stirred at room temperature for 16 hours, quenched with water, and extracted with ethyl acetate (3×50 mL). The combined organic phases were washed with water, brine, dried over sodium sulfate, and concentrated to give the title compound (2.0 g, crude) as a yellow solid. MS: 357 m/z [M+H]⁺.

The following intermediates were prepared utilizing the procedure described for Intermediate 6 and/or for Intermediate 6A.

| Inter. | | | MS m/z |
|---|---|---|---|
| No. | Structure | Name | [M + H]⁺ |
| 6-1 | 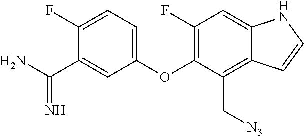 [SMILES: [N-]=[N+]=NCc1c(Oc2ccc(F)c(C(=N)N)c2)c(F)cc2[nH]ccc12] | 5-((4-(azidomethyl)-6-fluoro- 1H-indol-5-yl)oxy)-2- fluorobenzimidamide | 343 |
| 6-2 | 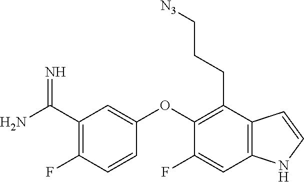 [SMILES: [N-]=[N+]=NCCCc1c(Oc2ccc(F)c(C(=N)N)c2)c(F)cc2[nH]ccc12] | 5-((4-(3-azidopropyl)-6-fluoro- 1H-indol-5-yl)oxy)-2- fluorobenzimidamide | 371 |
| 6-3 | 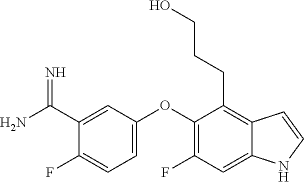 [SMILES: N=C(N)c1cc(Oc2c(F)cc3[nH]ccc3c2CCCO)ccc1F] | 2-fluoro-5-((6-fluoro-4-(3- hydroxypropyl)-1H-indol-5- yl)oxy)benzimidamide | 346 |
| 6-4 | 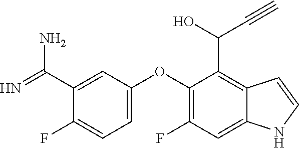 [SMILES: C#CC(O)c1c(Oc2ccc(F)c(C(=N)N)c2)c(F)cc2[nH]ccc12] | 2-fluoro-5-((6-fluoro-4-(1- hydroxyprop-2-yn-1-yl)-1H- indol-5-yl)oxy)benzimidamide | 364 [M + Na]⁺ |
| 6-5 | 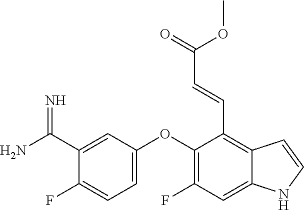 [SMILES: COC(=O)/C=C/c1c(Oc2ccc(F)c(C(=N)N)c2)c(F)cc2[nH]ccc12] | methyl (E)-3-(5-(3-carbamimidoyl- 4-fluorophenoxy)-6- fluoro-1H-indol-4-yl)acrylate | 372 |
| 6-6 | 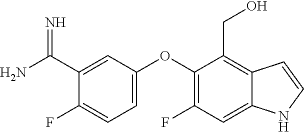 [SMILES: N=C(N)c1cc(Oc2c(F)cc3[nH]ccc3c2CO)ccc1F] | 2-fluoro-5-((6-fluoro-4- (hydroxymethyl)-1H-indol-5- yl)oxy)benzimidamide | 318 |
| 6-7 | 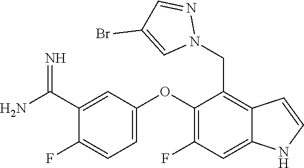 [SMILES: N=C(N)c1cc(Oc2c(F)cc3[nH]ccc3c2Cn2cc(Br)cn2)ccc1F] | 5-((4-((4-bromo-1H-pyrazol- 1-yl)methyl)-6-fluoro-1H- indol-5-yl)oxy)-2-fluorobenzimidamide | 446, 448 |
| 6-8 | 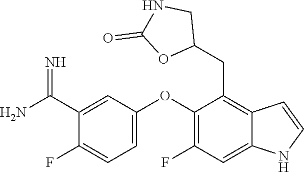 [SMILES: N=C(N)c1cc(Oc2c(F)cc3[nH]ccc3c2CC2CNC(=O)O2)ccc1F] | 2-fluoro-5-((6-fluoro-4-((2- oxooxazolidin-5-yl)methyl)- 1H-indol-5-yl)oxy)benzimidamide | 387 |
| 6-9 | 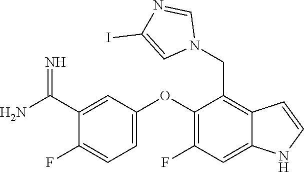 [SMILES: N=C(N)c1cc(Oc2c(F)cc3[nH]ccc3c2Cn2cnc(I)c2)ccc1F] | 2-fluoro-5-((6-fluoro-4- ((4-iodo-1H-imidazol-1- yl)methyl)-1H-indol-5-yl)oxy) benzimidamide | 494 |
| 6-10 | 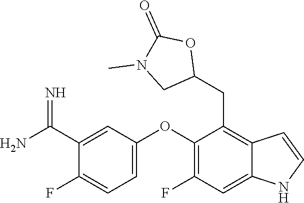 [SMILES: CN1CC(Cc2c(Oc3ccc(F)c(C(=N)N)c3)c(F)cc3[nH]ccc23)OC1=O] | 2-fluoro-5-((6-fluoro-4-((3- methyl-2-oxooxazolidin-5- yl)methyl)-1H-indol-5-yl)oxy) benzimidamide | 401 |
| 6-11 | 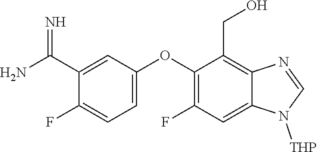 | 2-fluoro-5-((6-fluoro-4- (hydroxymethyl)-1-(tetrahydro-2H- pyran-2-yl)-1H-benzo[d]imidazol-5- yl)oxy)benzimidamide | 403 |
| 6-12 | 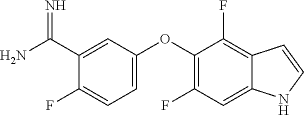 [SMILES: N=C(N)c1cc(Oc2c(F)cc3[nH]ccc3c2F)ccc1F] | 5-((4,6-difluoro-1H-indol-5-yl)oxy)-2- fluorobenzimidamide | 306 |
| 6-13 | 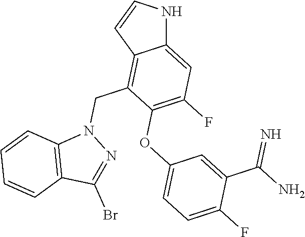 [SMILES: N=C(N)c1cc(Oc2c(F)cc3[nH]ccc3c2Cn2nc(Br)c3ccccc32)ccc1F] | 5-((4-((3-bromo-1H-indazol-1- yl)methyl)-6-fluoro-1H- indol-5-yl)oxy)-2-fluorobenzimidamide | 496, 498 |
| 6-14 | 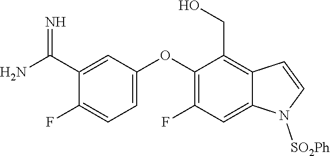 | 2-fluoro-5-((6-fluoro-4- (hydroxymethyl)-1- (phenylsulfonyl)-1H-indol-5-yl) oxy)benzimidamide | 458 |

### Intermediate 6A: 2-(5-(3-Cyano-4-fluorophenoxy)-6-fluoro-1-tosyl-1H-indol-4-yl)ethyl methanesulfonate

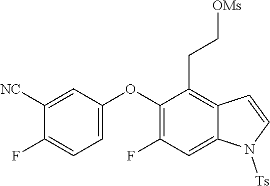 [SMILES: Cc1ccc(S(=O)(=O)n2ccc3c(CCOS(C)(=O)=O)c(Oc4ccc(F)c(C#N)c4)c(F)cc32)cc1]

To a stirred and cooled (0° C.) solution of 2-fluoro-5-((6-fluoro-4-(2-hydroxyethyl)-l-tosyl-l H-indol-5-yl)oxy)benzonitrile (Intermediate 5, 3.0 g, 6.40 mmol) and triethylamine (1.9 g, 19.20 mmol) in tetrahydrofuran (100 mL) was added methanesulfonyl chloride (1.5 g, 12.80 mmol). The mixture was stirred at room temperature for 1 hour, diluted with water and extracted with ethyl acetate (3×50 mL). The combined organic phases were washed with brine, dried over sodium sulfate, and concentrated to give the title compound (3.6 g, 100%) as yellow solid. MS: 547 m/z [M+H]⁺.

### Intermediate 6B: 5-((4-(2-Azidoethyl)-6-fluoro-1-tosyl-1H-indol-5-yl)oxy)-2-fluorobenzonitrile

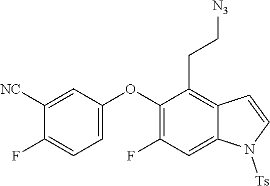 [SMILES: Cc1ccc(S(=O)(=O)n2ccc3c(CCN=[N+]=[N-])c(Oc4ccc(F)c(C#N)c4)c(F)cc32)cc1]

To a stirred solution of 2-(5-(3-cyano-4-fluorophenoxy)-6-fluoro-1-tosyl-1H-indol-4-yl)ethyl methanesulfonate (Intermediate 6A, 3.6 g, 6.59 mmol) in dimethylformamide (40 mL) was added sodium azide (1.3 g, 19.77 mmol). The mixture was heated at 70° C. for 2 hours, then cooled to room temperature. The mixture was partitioned between water (50 mL) and ethyl acetate (50 mL). The separated organic phase, combined with two additional ethyl acetate extracts (2×50 mL), was washed with water, brine, dried over sodium sulfate, and concentrated. The residue was purified by silica gel column chromatography (petroleum ether/ethyl acetate=2:1) to give the title compound (2.9 g, 89%) as a white solid. MS: 516 m/z [M+Na]⁺.

### Intermediate 7: 2-(3-Bromophenyl)-2-methylnon-8-ynoic acid

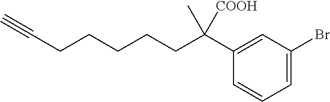 [SMILES: C#CCCCCCC(C)(C(=O)O)c1cccc(Br)c1]

To a suspension of methyl 2-(3-bromophenyl)-2-methylnon-8-ynoate (Intermediate 7A, 150 mg, 0.44 mmol) in 2 mL of methanol and 1 mL water was added lithium hydroxide monohydrate (356 mg, 8.9 mmol). The mixture was stirred at room temperature for 18 hours and concentrated. The residue was acidified to pH ˜4 with 1 N hydrochloric acid and extracted with ethyl acetate (2×20 mL). The combined organic extracts were washed with brine, dried over sodium sulfate, and concentrated to give the crude title product (140 mg, 90%) as a white solid, which was used for next step without further purification. MS (ESI): 323, 325 m/z [M+H]⁺.

The following intermediates were prepared utilizing the procedure described for Intermediate 7 and/or for Intermediate 7A.

| Inter. | | | MS m/z |
|---|---|---|---|
| No. | Structure | Name | [M + H]⁺ |
| 7-1 | 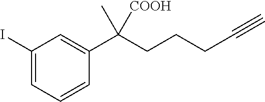 [SMILES: C#CCCCC(C)(C(=O)O)c1cccc(I)c1] | 2-(3-iodophenyl)-2- methylhept-6-ynoic acid | 343 |
| 7-2 | 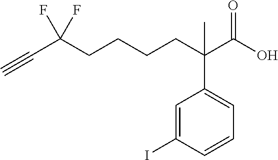 [SMILES: C#CC(F)(F)CCCCC(C)(C(=O)O)c1cccc(I)c1] | 7,7-difluoro-2-(3-iodophenyl)- 2-methylnon-8-ynoic acid | 429 [M + Na]⁺ |
| 7-3 | 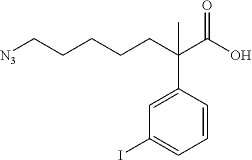 [SMILES: CC(CCCCCN=[N+]=[N-])(C(=O)O)c1cccc(I)c1] | 7-azido-2-(3-iodophenyl)-2- methylheptanoic acid | 410 [M + Na]⁺ |
| 7-4 | 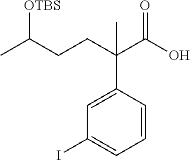 [SMILES: CC(CCC(C)(C(=O)O)c1cccc(I)c1)O[Si](C)(C)C(C)(C)C] | 5-((tert- butyldimethylsilyl)oxy)-2-(3- iodophenyl)-2-methylhexanoic acid | 485 [M + Na]⁺ |
| 7-5 | 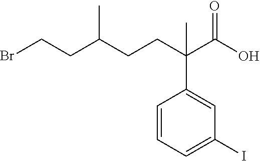 [SMILES: CC(CCBr)CCC(C)(C(=O)O)c1cccc(I)c1] | 7-bromo-2-(3-iodophenyl)- 2,5-dimethylheptanoic acid | 439, 441 |
| 7-6 | 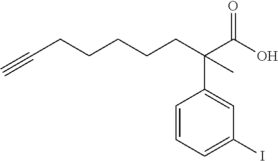 [SMILES: C#CCCCCCC(C)(C(=O)O)c1cccc(I)c1] | 2-(3-iodophenyl)-2- methylnon-8-ynoic acid | 371 |
| 7-7 | 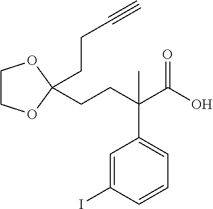 [SMILES: C#CCCC1(CCC(C)(C(=O)O)c2cccc(I)c2)OCCO1] | 4-(2-(but-3-yn-1-yl)-1,3- dioxolan-2-yl)-2-(3- iodophenyl)-2-methylbutanoic acid | 429 |
| 7-8 | 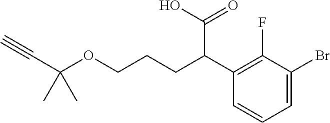 [SMILES: C#CC(C)(C)OCCCC(C(=O)O)c1cccc(Br)c1F] | 2-(3-bromo-2-fluorophenyl)- 5-((2-methylbut-3-yn-2- yl)oxy)pentanoic acid | 379, 381 [M + Na]⁺ |
| 7-9 | 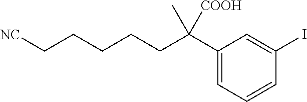 [SMILES: CC(CCCCCC#N)(C(=O)O)c1cccc(I)c1] | 7-cyano-2-(3-iodophenyl)-2- methylheptanoic acid | 394 [M + Na]⁺ |
| 7-10 | 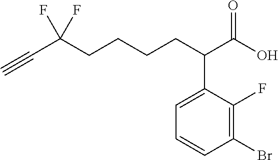 [SMILES: C#CC(F)(F)CCCCC(C(=O)O)c1cccc(Br)c1F] | 2-(3-bromo-2-fluorophenyl)- 7,7-difluoronon-8-ynoic acid | 385, 387 [M + Na]⁺ |
| 7-11 | 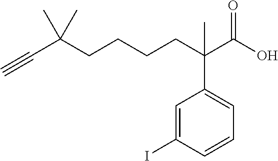 [SMILES: C#CC(C)(C)CCCCC(C)(C(=O)O)c1cccc(I)c1] | 2-(3-iodophenyl)-2,7,7- trimethylnon-8-ynoic acid | MS: 421 m/z [M + Na]⁺; ¹H NMR (400 MHz, CDCl₃) δ 7.72 (s, 1H), 7.62 (d, J = 7.8 Hz, 1H), 7.37 (d, J = 8.0 Hz, 1H), 7.09 (t, J = 7.9 Hz, 1H), 2.07-1.88 (m, 2H), 1.95-1.90 (m, 1H), 1.57 (s, 3H), 1.52- 1.43 (m, 2H), 1.43-1.35 (m, 2H), 1.31- 1.16 (m, 8H) ppm. |
| 7-12 | 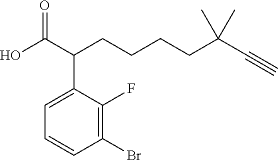 [SMILES: C#CC(C)(C)CCCCC(C(=O)O)c1cccc(Br)c1F] | 2-(3-bromo-2-fluorophenyl)- 7,7-dimethylnon-8-ynoic acid | 355, 357 |
| 7-13 | 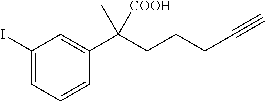 [SMILES: C#CCCCC(C)(C(=O)O)c1cccc(I)c1] | 2-(3-iodophenyl)-2- methylheptyl-6-ynoic acid | 343 |
| 7-14 | 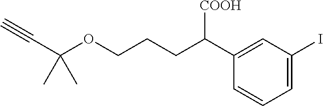 [SMILES: C#CC(C)(C)OCCCC(C(=O)O)c1cccc(I)c1] | 2-(3-iodophenyl)-5-((2- methylbut-3-yn-2- yl)oxy)pentanoic acid | MS: 409 [M + Na]⁺; ¹H NMR (400 MHz, CDCl₃) δ 7.67 (t, J = 1.6 Hz, 1H), 7.64- 7.56 (m, 1H), 7.32-7.27 (m, 1H), 7.06 (t, J = 8.0 Hz, 1H), 3.63-3.42 (m, 3H), 2.39 (s, 1H), 2.23-2.07 (m, 1H), 1.91-1.80 (m, 1H), 1.63-1.49 (m, 2H), 1.44 (s, 6H) ppm. |
| 7-15 | 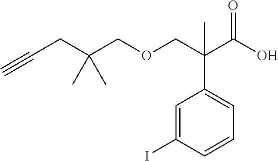 [SMILES: C#CCC(C)(C)COCC(C)(C(=O)O)c1cccc(I)c1] | 3-((2,2-dimethylpent-4-yn-1- yl)oxy)-2-(3-iodophenyl)-2- methylpropanoic acid | 423 [M + Na]⁺ |
| 7-16 | 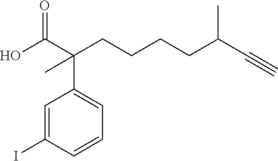 [SMILES: C#CC(C)CCCCC(C)(C(=O)O)c1cccc(I)c1] | 2-(3-iodophenyl)-2,7- dimethylnon-8-ynoic acid | 385 |
| 7-17 | 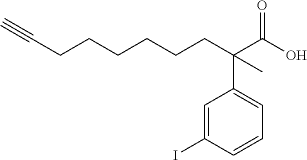 [SMILES: C#CCCCCCCC(C)(C(=O)O)c1cccc(I)c1] | 2-(3-iodophenyl)-2- methyldec-9-ynoic acid | 385 |
| 7-18 | 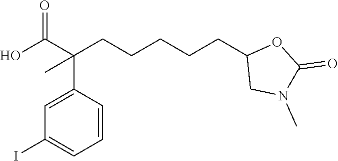 [SMILES: CN1CC(CCCCCC(C)(C(=O)O)c2cccc(I)c2)OC1=O] | 2-(3-iodophenyl)-2-methyl-7- (3-methyl-2-oxooxazolidin-5- yl)heptanoic acid | 446 |
| 7-19 | 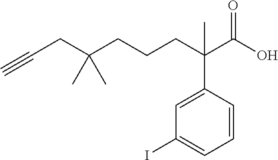 [SMILES: C#CCC(C)(C)CCCC(C)(C(=O)O)c1cccc(I)c1] | 2-(3-iodophenyl)-2,6,6- trimethylnon-8-ynoic acid | 399 |
| 7-20 | 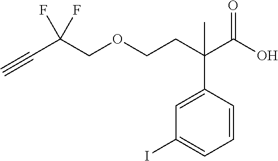 [SMILES: C#CC(F)(F)COCCC(C)(C(=O)O)c1cccc(I)c1] | 4-((2,2-difluorobut-3-yn-1- yl)oxy)-2-(3-iodophenyl)-2- methylbutanoic acid | 431 |
| 7-20A | 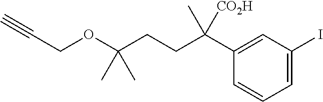 [SMILES: C#CCOC(C)(C)CCC(C)(C(=O)O)c1cccc(I)c1] | 2-(3-iodophenyl)-2,5- dimethyl-5-(prop-2-yn-1- yloxy)hexanoic acid | 423 [M + Na]⁺ |
| 7-21 | 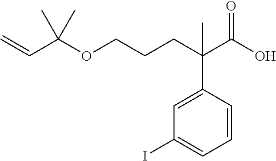 [SMILES: C=CC(C)(C)OCCCC(C)(C(=O)O)c1cccc(I)c1] | 2-(3-iodophenyl)-2-methyl-5- ((2-methylbut-3-en-2- yl)oxy)pentanoic acid | 425 [M + Na]⁺ |
| 7-22 | 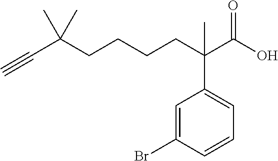 [SMILES: C#CC(C)(C)CCCCC(C)(C(=O)O)c1cccc(Br)c1] | 2-(3-bromophenyl)-2,7,7- trimethylnon-8-ynoic acid | 373, 375 [M + Na]⁺ |
| 7-23 | 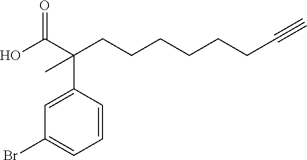 [SMILES: C#CCCCCCCC(C)(C(=O)O)c1cccc(Br)c1] | 2-(3-bromophenyl)-2- methyldec-9-ynoic acid | 337, 339 |
| 7-24 | 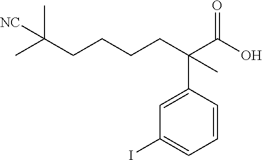 [SMILES: CC(C)(C#N)CCCCC(C)(C(=O)O)c1cccc(I)c1] | 7-cyano-2-(3-iodophenyl)-2,7- dimethyloctanoic acid | 354 [M − CO2]⁺ |
| 7-25 | 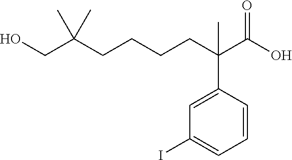 [SMILES: CC(C)(CO)CCCCC(C)(C(=O)O)c1cccc(I)c1] | 8-hydroxy-2-(3-iodophenyl)- 2,7,7-trimethyloctanoic acid | 427 [M + Na]⁺ |
| 7-26 | 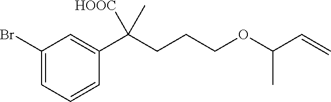 [SMILES: C=CC(C)OCCCC(C)(C(=O)O)c1cccc(Br)c1] | 2-(3-bromophenyl)-5-(but-3- en-2-yloxy)-2- methylpentanoic acid | 363, 365 [M + Na]⁺ |
| 7-27 | 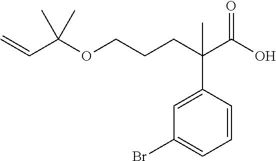 [SMILES: C=CC(C)(C)OCCCC(C)(C(=O)O)c1cccc(Br)c1] | 2-(3-bromophenyl)-2-methyl- 5-((2-methylbut-3-en-2- yl)oxy)pentanoic acid | 377, 379 [M + Na]⁺ |
| 7-28 | 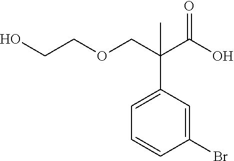 [SMILES: CC(COCCO)(C(=O)O)c1cccc(Br)c1] | 2-(3-bromophenyl)-3-(2- hydroxyethoxy)-2- methylpropanoic acid | 303, 305 |
| 7-29 | 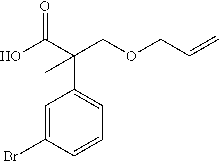 [SMILES: C=CCOCC(C)(C(=O)O)c1cccc(Br)c1] | 3-(allyloxy)-2-(3- bromophenyl)-2- methylpropanoic acid | 321, 323 [M + Na]⁺ |
| 7-30 | 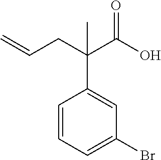 | 2-(3-bromophenyl)-2- methylpent-4-enoic acid | 269, 271 |
| 7-31 | 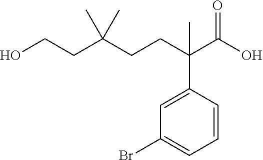 [SMILES: CC(C)(CCO)CCC(C)(C(=O)O)c1cccc(Br)c1] | 2-(3-bromophenyl)-7- hydroxy-2,5,5- trimethylheptanoic acid | 343, 345 |
| 7-32 | 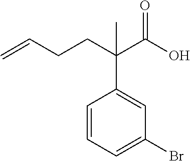 [SMILES: C=CCCC(C)(C(=O)O)c1cccc(Br)c1] | 2-(3-bromophenyl)-2- methylhex-5-enoic acid | 283, 285 |
| 7-33 | 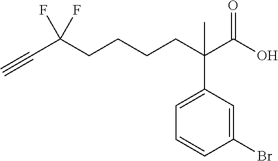 [SMILES: C#CC(F)(F)CCCCC(C)(C(=O)O)c1cccc(Br)c1] | 2-(3-bromophenyl)-7,7- difluoro-2-methylnon-8-ynoic acid | 381, 383 [M + Na]⁺ |
| 7-34 | 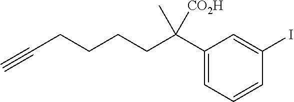 [SMILES: C#CCCCCC(C)(C(=O)O)c1cccc(I)c1] | 2-(3-iodophenyl)-2-methyloct- 7-ynoic acid | 357 |
| 7-35 | 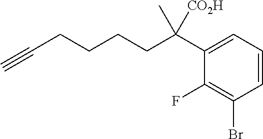 [SMILES: C#CCCCCC(C)(C(=O)O)c1cccc(Br)c1F] | 2-(3-bromo-2-fluorophenyl)- 2-methyloct-7-ynoic acid | 327, 329 |
| 7-36 | 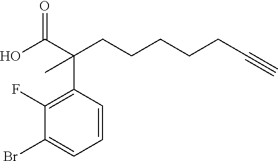 [SMILES: C#CCCCCCC(C)(C(=O)O)c1cccc(Br)c1F] | 2-(3-bromo-2-fluorophenyl)- 2-methylnon-8-ynoic acid | 341, 343 |
| 7-37 | 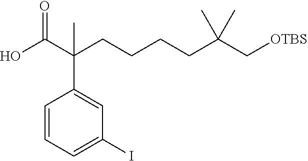 [SMILES: CC(C)(CCCCC(C)(C(=O)O)c1cccc(I)c1)CO[Si](C)(C)C(C)(C)C] | 8-((tert- butyldimethylsilyl)oxy)-2-(3- iodophenyl)-2,7,7- trimethyloctanoic acid | 519 |
| 7-38 | 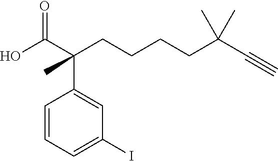 [SMILES: C#CC(C)(C)CCCC[C@@](C)(C(=O)O)c1cccc(I)c1] | (R)-2-(3-iodophenyl)-2,7,7- trimethylnon-8-ynoic acid | 399 |

### Intermediate 7A: Methyl 2-(3-bromophenyl)-2-methylnon-8-ynoate

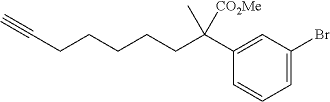 [SMILES: C#CCCCCCC(C)(C(=O)OC)c1cccc(Br)c1]

To a stirred and cooled (−78° C.) solution of methyl 2-(3-bromophenyl)propanoate (222 mg, 1 mmol) in 10 mL of anhydrous tetrahydrofuran was added lithium diisopropylamide (0.75 mL, 1.5 mmol) dropwise. The mixture was stirred for 30 minutes, then treated with 7-iodohept-1-yne (290 mg, 1.2 mmol) dropwise at this temperature. The mixture was slowly allowed to warm to room temperature and stirred for another 1 hour, quenched with saturated ammonium chloride solution and extracted with ethyl acetate (2×50 mL). The combined organic layers were washed with brine, dried over sodium sulfate, and concentrated. The residue was purified by silica gel column (eluting with 5% ethyl acetate/petroleum ether to provide the title compound (150 mg, 40%) as light oil. MS: 337, 339 m/z [M+H]⁺.

The following intermediates were prepared utilizing the procedure described for Intermediate 7A.

| Inter. | | | MS m/z |
|---|---|---|---|
| No. | Structure | Name | [M + H]⁺ |
| 7A-1 | 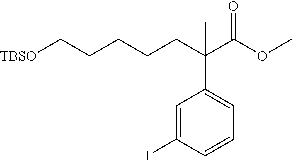 [SMILES: COC(=O)C(C)(CCCCCO[Si](C)(C)C(C)(C)C)c1cccc(I)c1] | methyl 7-((tert- butyldimethylsilyl)oxy)- 2-(3-iodophenyl)-2- methylheptanoate | 491 |
| 7A-2 | 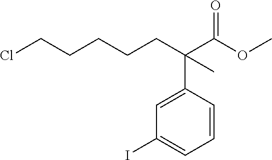 [SMILES: COC(=O)C(C)(CCCCCCl)c1cccc(I)c1] | methyl 7-chloro-2- (3-iodophenyl)-2- methylheptanoate | MS: 417 m/z [M + H]⁺; ¹H NMR (400 MHz, CDCl₃) δ 7.64-7.58 (m, 2H), 7.28-7.26 (m, 1H), 7.09-7.05 (m, 1H), 3.68 (s, 3H), 3.53-3.50 (m, 2H), 2.07-1.74 (m, 4H), 1.53 (s, 3H), 1.50-1.42 (m, 2H), 1.27-1.16 (m, 2H) ppm. |
| 7A-3 | 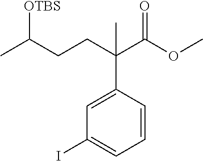 [SMILES: COC(=O)C(C)(CCC(C)O[Si](C)(C)C(C)(C)C)c1cccc(I)c1] | methyl 5-((tert- butyldimethylsilyl)oxy)- 2-(3-iodophenyl)-2- methylhexanoate | 499 |
| 7A-4 | 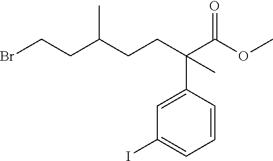 [SMILES: COC(=O)C(C)(CCC(C)CCBr)c1cccc(I)c1] | methyl 7-bromo-2- (3-iodophenyl)-2,5- dimethylheptanoate | 475, 477 [M + Na]⁺ |
| 7A-5 | 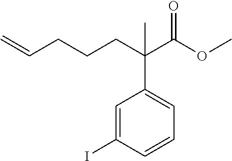 [SMILES: C=CCCCC(C)(C(=O)OC)c1cccc(I)c1] | methyl 2-(3-iodophenyl)-2- methylhept-6-enoate | 359 |
| 7A-6 | 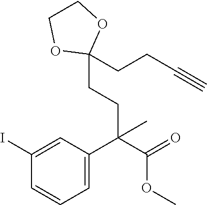 [SMILES: C#CCCC1(CCC(C)(C(=O)OC)c2cccc(I)c2)OCCO1] | methyl 4-(2-(but-3-yn-1-yl)- 1,3-dioxolan-2-yl)-2-(3- iodophenyl)-2- methylbutanoate | MS: 443 [M + H]+. ¹H NMR (400 MHz, CDCl₃) δ 7.63 (t, J = 2.0 Hz, 1H), 7.59-7.56 (m, 1H), 7.27-7.25 (m, 1H), 7.05 (t, J = 8.0 Hz, 1H), 3.93-3.86 (m, 4H), 3.66 (s, 3H), 2.25-2.20 (m, 2H), 2.06-1.98 (m, 2H), 1.92 (t, J = 2.8 Hz, 1H), 1.90-1.87 (m, 2H), 1.51 (s, 3H), 1.49-1.43 (m, 2H) ppm |
| 7A-7 | 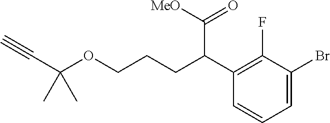 [SMILES: C#CC(C)(C)OCCCC(C(=O)OC)c1cccc(Br)c1F] | methyl 2-(3-bromo- 2-fluorophenyl)-5- ((2-methylbut-3-yn-2- yl)oxy)pentanoate | 393, 395 [M + Na]⁺ |
| 7A-8 | 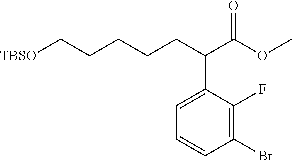 [SMILES: COC(=O)C(CCCCCO[Si](C)(C)C(C)(C)C)c1cccc(Br)c1F] | methyl 2-(3-bromo- 2-fluorophenyl)-7- ((tert-butyldimethylsilyl) oxy)heptanoate | 447, 449 |
| 7A-9 | 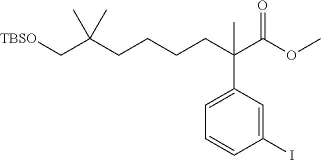 [SMILES: COC(=O)C(C)(CCCCC(C)(C)CO[Si](C)(C)C(C)(C)C)c1cccc(I)c1] | methyl 8-((tert- butyldimethylsilyl)oxy)-2-(3- iodophenyl)-2,7,7- trimethyloctanoate | 1H NMR (400 MHz, CDCl₃) δ 7.62 (t, J = 1.7 Hz, 1H), 7.57- 7.54 (m, 1H), 7.27-7.22 (m, 1H), 7.03 (t, J = 7.9 Hz, 1H), 3.64 (s, 3H), 3.19 (s, 2H), 2.02- 1.77 (m, 2H), 1.24-1.08 (m, 6H), 0.87 (s, 9H), 0.78 (s, 6H), 0.001 (s, 6H) ppm. |
| 7A-10 | 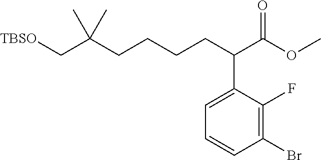 [SMILES: COC(=O)C(CCCCC(C)(C)CO[Si](C)(C)C(C)(C)C)c1cccc(Br)c1F] | methyl 2-(3-bromo-2- fluorophenyl)-8- ((tert-butyldimethylsilyl) oxy)-7,7- dimethyloctanoate | ¹H NMR (400 MHz, CDCl₃) δ 7.46-7.42 (m, 1H), 7.35-7.22 (m, 1H), 7.02-6.98 m, 1H), 3.92 (t, J = 7.7 Hz, 1H), 3.67 (s, 3H), 3.19 (s, 2H), 2.10-1.56 (m, 2H), 1.30-1.08 (m, 6H), 0.87 (s, 9H), 0.78 (s, 6H), 0.00 (s, 6H) ppm. |
| 7A-11 | 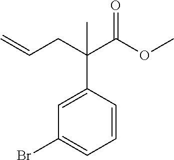 [SMILES: C=CCC(C)(C(=O)OC)c1cccc(Br)c1] | methyl 2-(3- bromophenyl)-2- methylpent-4-enoate | 283, 285 |
| 7A-12 | 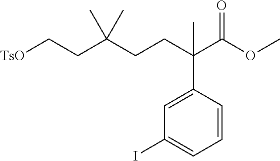 [SMILES: COC(=O)C(C)(CCC(C)(C)CCOS(=O)(=O)c1ccc(C)cc1)c1cccc(I)c1] | methyl 2-(3- iodophenyl)-2,5,5- trimethyl-7-(tosyloxy) heptanoate | ¹H NMR (400 MHz, CDCl₃) δ 7.78 (d, J = 8.3 Hz, 2H), 7.59 (d, J = 1.3 Hz, 2H), 7.34 (d, J = 8.0 Hz, 2H), 7.25-7.20 (m, 1H), 7.08-7.03 (m, 1H), 4.04 (t, J = 7.4 Hz, 2H), 3.65 (s, 3H), 2.44 (s, 3H), 1.95-1.86 (m, 1H), 1.79-1.70 (m, 1H), 1.64-1.57 (m, 2H), 1.46 (s, 3H), 1.02-0'97 (m, 2H), 0.83 (s, 6H) ppm. |
| 7A-13 | 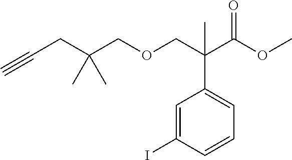 [SMILES: C#CCC(C)(C)COCC(C)(C(=O)OC)c1cccc(I)c1] | methyl 3-((2,2- dimethylpent- 4-yn-1-yl)oxy)- 2-(3-iodophenyl)-2- methylpropanoate | 437 [M + Na]⁺ |
| 7A-14 | 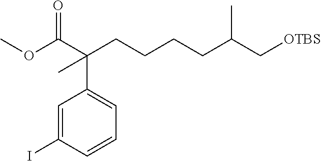 [SMILES: COC(=O)C(C)(CCCCC(C)CO[Si](C)(C)C(C)(C)C)c1cccc(I)c1] | methyl 8-((tert- butyldimethylsilyl)oxy)- 2-(3-iodophenyl)-2,7- dimethyloctanoate | 519 |
| 7A-15 | 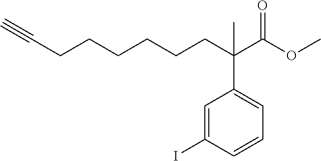 [SMILES: C#CCCCCCCC(C)(C(=O)OC)c1cccc(I)c1] | methyl 2-(3-iodophenyl)- 2-methyldec- 9-ynoate | 1H NMR (400 MHz, CDCl₃) δ 7.63-7.56 (m, 2H), 7.25-7.23 (m, 1H), 7.05 (t, J = 7.9 Hz, 1H), 3.66 (s, 3H), 2.16-2.14 (m, 2H), 2.06-1.81 (m, 3H), 1.55- 1.45 (m, 5H), 1.40-1.29 (m, 4H), 1.21-1.14 (m, 2H) ppm. |
| 7A-16 | 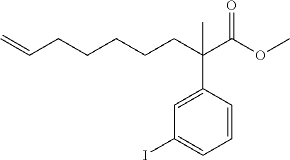 [SMILES: C=CCCCCCC(C)(C(=O)OC)c1cccc(I)c1] | methyl 2-(3-iodophenyl)-2- methylnon-8-enoate | 409 [M + Na]⁺ |
| 7A-17 | 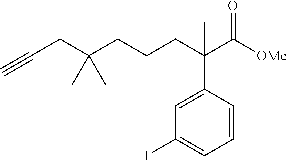 [SMILES: C#CCC(C)(C)CCCC(C)(C(=O)OC)c1cccc(I)c1] | methyl 2-(3-iodophenyl)- 2,6,6- trimethylnon-8-ynoate | MS: 435 [M + Na]⁺; ¹H NMR (400 MHz, CHCl₃) δ 7.63 (s, 1H), 7.57 (d, J = 8.0 Hz, 1H), 7.26-7.25 (m, 1H), 7.05 (t, J = 8.0 Hz, 1H), 3.66 (s, 3H), 2.03- 1.95 (m, 4H), 1.85-1.78 (m, 1H), 1.53 (s, 3H), 1.32-1.07 (m, 4H), 0.92 (s, 6H) ppm. |
| 7A-18 | 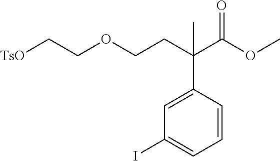 [SMILES: COC(=O)C(C)(CCOCCOS(=O)(=O)c1ccc(C)cc1)c1cccc(I)c1] | methyl 2-(3-iodophenyl)- 2-methyl-4- (2-(tosyloxy)ethoxy) butanoate | 555 [M + Na]⁺ |
| 7A-19 | 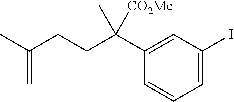 [SMILES: C=C(C)CCC(C)(C(=O)OC)c1cccc(I)c1] | methyl 2-(3-iodophenyl)- 2,5- dimethylhex-5-enoate | ¹H NMR (400 MHz, CDCl₃) δ 7.64 (t, J = 1.8 Hz, 1H), 7.60- 7.57 (m, 1H), 7.29-7.27 (m, 1H), 7.06 (t, J = 7.9 Hz, 1H), 4.73-4.67 (m, 2H), 3.69-3.65 (m, 3H), 2.23-2.10 (m, 1H), 2.06-1.93 (m, 1H), 1.91-1.81 |
| | | | (m, 2H), 1.71 (s, 3H), 1.55-1.53 |
| | | | (m, 3H) ppm. |
| 7A-20 | 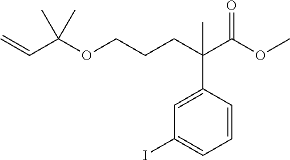 [SMILES: C=CC(C)(C)OCCCC(C)(C(=O)OC)c1cccc(I)c1] | methyl 2-(3-iodophenyl)- 2-methyl-5- ((2-methylbut-3-en-2- yl)oxy)pentanoate | 439 [M + Na]⁺ |
| 7A-21 | 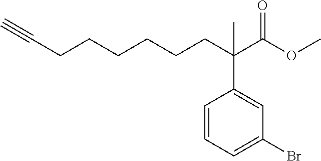 [SMILES: C#CCCCCCCC(C)(C(=O)OC)c1cccc(Br)c1] | methyl 2-(3- bromophenyl)-2- methyldec-9-ynoate | 373, 375 [M + Na]⁺ |
| 7A-22 | 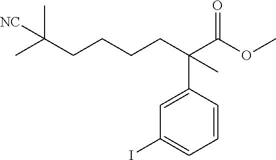 [SMILES: COC(=O)C(C)(CCCCC(C)(C)C#N)c1cccc(I)c1] | methyl 7-cyano-2-(3- iodophenyl)-2,7- dimethyloctanoate | 436 [M + Na]⁺ |
| 7A-23 | 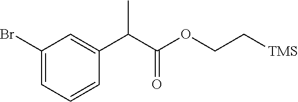 [SMILES: CC(C(=O)OCC[Si](C)(C)C)c1cccc(Br)c1] | 2-(trimethylsilyl)ethyl 2-(3- bromophenyl)propanoate | 367, 369 [M + K]⁺ |
| 7A-24 | 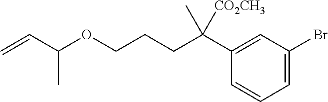 [SMILES: C=CC(C)OCCCC(C)(C(=O)OC)c1cccc(Br)c1] | methyl 2-(3-bromophenyl)- 5-(but-3-en-2-yloxy)- 2-methylpentanoate | 337, 379 [M + Na]⁺ |
| 7A-25 | 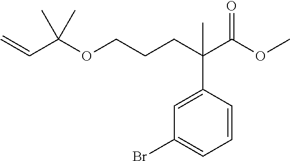 [SMILES: C=CC(C)(C)OCCCC(C)(C(=O)OC)c1cccc(Br)c1] | methyl 2-(3-bromophenyl)- 2-methyl- 5-((2-methylbut-3-en-2- yl)oxy)pentanoate | 391, 393 [M + Na]⁺ |
| 7A-26 | 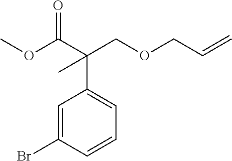 [SMILES: C=CCOCC(C)(C(=O)OC)c1cccc(Br)c1] | methyl 3-(allyloxy)-2-(3- bromophenyl)-2- methylpropanoate | MS: 313, 315 [M + H]+; ¹H NMR (400 MHz, CDCl₃) δ 7.46 (s, 1H), 7.39 (d, J = 7.6 Hz, 1H), 7.25 (d, J = 8.4 Hz, 1H), 7.18 (d, J = 7.6 Hz, 1H), 5.90- 5.82 (m, 1H), 5.35-5.04 (m, 2H), 4.04-3.97 (m, 2H), 3.96- 3.92 (m, 1H), 3.69 (s, 3H), 3.67- 3.63 (m, 1H), 1.63 (s, 3H) ppm. |
| 7A-27 | 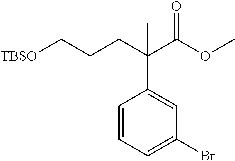 [SMILES: COC(=O)C(C)(CCCO[Si](C)(C)C(C)(C)C)c1cccc(Br)c1] | methyl 2-(3-bromophenyl)- 5-((tert- butyldimethylsilyl)oxy)-2- methylpentanoate | 415, 417 |
| 7A-28 | 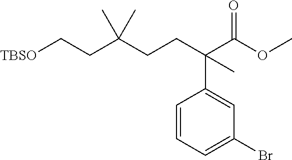 [SMILES: COC(=O)C(C)(CCC(C)(C)CCO[Si](C)(C)C(C)(C)C)c1cccc(Br)c1] | methyl 2-(3-bromophenyl)- 7-((tert-butyl- dimethylsilyl)oxy)-2,5,5- trimethylheptanoate | 471, 473 |
| 7A-29 | 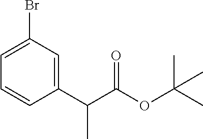 [SMILES: CC(C(=O)OC(C)(C)C)c1cccc(Br)c1] | tert-butyl 2-(3- bromophenyl)propanoate | 307, 309 [M + Na]⁺ |
| 7A-30 | 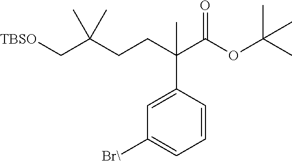 | tert-butyl 2-(3- bromophenyl)-6-((tert- butyldimethylsilyl)oxy)- 2,5,5- trimethylhexanoate | 521, 523 [M + Na]⁺ |
| 7A-31 | 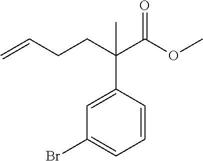 [SMILES: C=CCCC(C)(C(=O)OC)c1cccc(Br)c1] | methyl 2-(3-bromophenyl)- 2-methylhex-5-enoate | 297, 299 |
| 7A-32 | 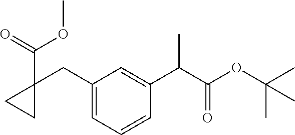 [SMILES: COC(=O)C1(Cc2cccc(C(C)C(=O)OC(C)(C)C)c2)CC1] | methyl 1-(3-(1- (tert-butoxy)-1- oxopropan-2-yl)benzyl) cyclopropane- 1-carboxylate | 341 [M + Na]⁺ |
| 7A-33 | 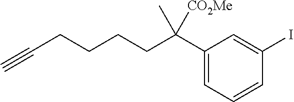 [SMILES: C#CCCCCC(C)(C(=O)OC)c1cccc(I)c1] | methyl 2-(3-iodophenyl)- 2-methyloct- 7-ynoate | 371 |
| 7A-34 | 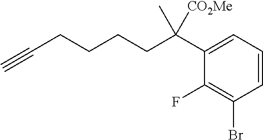 [SMILES: C#CCCCCC(C)(C(=O)OC)c1cccc(Br)c1F] | methyl 2-(3-bromo-2- fluorophenyl)-2- methyloct-7-ynoate | 341, 343 |
| 7A-35 | 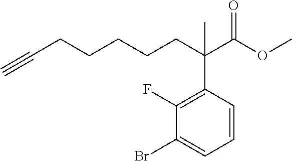 [SMILES: C#CCCCCCC(C)(C(=O)OC)c1cccc(Br)c1F] | methyl 2-(3-bromo-2- fluorophenyl)-2- methylnon-8-ynoate | 355, 357 |
| 7A-36 | 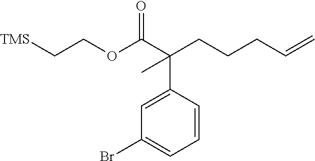 [SMILES: C=CCCCC(C)(C(=O)OCC[Si](C)(C)C)c1cccc(Br)c1] | 2-(trimethylsilyl)ethyl 2-(3-bromophenyl)- 2-methylhept- 6-enoate | ¹H NMR (400 MHz, CDCl₃) δ 7.51-7.45 (m, 1H), 7.42-7.34 (m, 1H), 7.27-7.16 (m, 2H), 5.81- 5.73 (m, 1H), 5.08-4.92 (m, 2H), 4.23-4.10 (m, 2H), 2.14-1.77 (m, 4H), 1.54 (s, 3H), 1.34-1.22 (m, 2H), 0.98-0.88 (m, 2H), 0.22 (s, 9H). |
| 7A-37 | 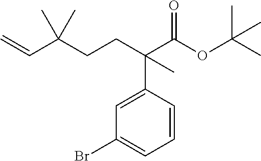 [SMILES: C=CC(C)(C)CCC(C)(C(=O)OC(C)(C)C)c1cccc(Br)c1] | tert-butyl 2-(3- bromophenyl)-2,5,5- trimethylhept-6-enoate | 403, 405 [M + Na]⁺ |

### Intermediate 8: 5-((4-(Bromomethyl)-6-fluoro-1-(phenylsulfonyl)-1H-indol-5-yl)oxy)-2-fluorobenzonitrile

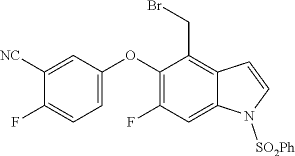

To a solution of 2-fluoro-5-((6-fluoro-4-methyl-1-(phenylsulfonyl)-1H-indol-5-yl)oxy)benzonitrile (Intermediate 42B-4, 8 g, 18.8 mmol) in dry carbon tetrachloride (650 ml) were added N-bromosuccinimide (3.7 g, 20.7 mmol) and azobisisobutyronitrile (0.92 g, 5.6 mmol) at room temperature. The reaction mixture was stirred at 80° C. for five hours, quenched with saturated potassium carbonate solution (200 ml) and extracted with ethyl acetate (100 ml×3). The combined organic extracts were washed with brine, dried over sodium sulfate, and concentrated. The residue was triturated with diethyl ether (50 mL), then purified by automated flash chromatography (80 g silica gel column, eluting with 0-50% ethyl acetate in petroleum ether) to give the title compound as a brown solid (6.4 g, 68%). MS (ESI): 503, 505 m/z [M+H]⁺, retention time: 1.87 minutes, purity: 95% (254 nm) (LC-MS method 4). ¹H NMR (400 MHz, CDCl₃) δ 7.94 (d, J=7.6 Hz, 2H), 7.86 (d, J=10.4 Hz, 1H), 7.72 (d, J=3.6 Hz, 1H), 7.64 (m, 1H), 7.55 (m, 2H), 7.19-7.16 (m, 1H), 7.13 (m, 1H), 7.08-7.04 (m, 1H), 6.82 (d, J=3.6 Hz, 1H), 4.64 (s, 2H) ppm.

The following intermediate was prepared based on the procedures described for Intermediate 8.

| Inter. | | | MS m/z |
|---|---|---|---|
| No. | Structure | Name | [M + H]⁺ |
| 8-1 | 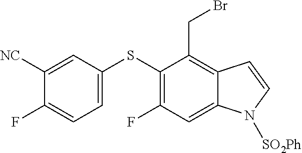 | 5-((4-(bromomethyl)-6-fluoro- 1-(phenylsulfonyl)-1H- indol-5-yl)thio)-2-fluorobenzonitrile | 519, 521 |

### Intermediate 9: 5-((4-(Azidomethyl)-6-fluoro-1H-indol-5-yl)oxy)-2-fluorobenzonitrile

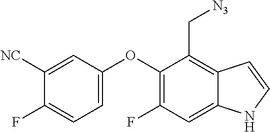 [SMILES: N#Cc1cc(Oc2c(F)cc3[nH]ccc3c2CN=[N+]=[N-])ccc1F]

To a stirred solution of 5-((4-(azidomethyl)-6-fluoro-1-(phenylsulfonyl)-1H-indol-5-yl)oxy)-2-fluorobenzonitrile (Intermediate 9A, 5.5 g, 11.8 mmol) in methanol (100 mL) was added potassium carbonate (4.9 g, 35.5 mmol) at room temperature. The reaction was stirred at 60° C. for one hour, then concentrated to remove methanol. The mixture was partitioned between water (100 mL) and ethyl acetate (200 mL). The separated organic layer was washed with brine, dried over anhydrous sodium sulfate, and concentrated. The residue was purified by automated flash chromatography (80 g silica gel column, eluting with 0-50% ethyl acetate in petroleum ether) to give the title compound (1.5 g, 40%) as a solid. MS (ESI): 326 m/z [M+H]⁺, retention time: 2.02 minutes, purity: 95% (254 nm) (LC-MS method 5).

### Intermediate 9A: 5-((4-(Azidomethyl)-6-fluoro-1-(phenylsulfonyl)-1H-indol-5-yl)oxy)-2-fluorobenzonitrile

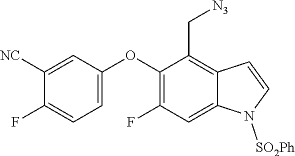

To a stirred solution of 5-((4-(bromomethyl)-6-fluoro-1-(phenylsulfonyl)-1H-indol-5-yl)oxy)-2-fluorobenzonitrile (Intermediate 8, 6.4 g, 12.7 mmol) in N,N-dimethylformamide (100 mL) was added sodium azide (1.7 g, 25.4 mmol) at room temperature. The reaction was stirred at room temperature for 16 hours, then diluted with ethyl acetate (300 mL). The solution was washed with brine, dried over anhydrous sodium sulfate, and concentrated. The residue was purified by automated flash chromatography (80 g silica gel column, eluting with 0-50% ethyl acetate in petroleum ether) to give the title compound (5.5 g, 92%) as an oil. MS (ESI): 466 m/z [M+H]⁺, retention time: 1.92 minutes, purity: 95% (254 nm) (LC-MS method 4).

### Intermediate 10: 5-((4-Allyl-6-fluoro-1H-indol-5-yl)oxy)-2-fluorobenzonitrile

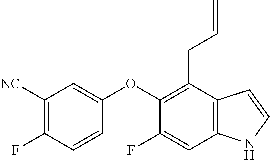 [SMILES: C=CCc1c(Oc2ccc(F)c(C#N)c2)c(F)cc2[nH]ccc12]

To a stirred and degassed solution of 5-[(4-bromo-6-fluoro-1H-indol-5-yl)oxy]-2-fluoro-benzonitrile (Intermediate 3, 10 g, 0.029 mol) in N,N′-dimethylformamide (100 mL) was added allyl(tributyl)stannane (11.4 g, 0.034 mol), lithium chloride (3.6 g, 0.086 mol), and bis-(triphenylphosphine)palladium(II) chloride (1.21 g, 1.72 mmol). The mixture was stirred at 90° C. under nitrogen overnight, then cooled to room temperature and treated with saturated potassium fluoride (300 mL). The solution was stirred for 10 minutes and filtered to remove the solid. The filtrate was extracted with ethyl acetate (3×200 mL). The combined organic extracts were washed with saturated lithium chloride solution, brine, dried over sodium sulfate, and concentrated. The residue was purified by flash column chromatography (ethyl acetate: petroleum ether=1:3) to give the title compound (9.5 g, 65%) as a light pink solid. MS (ESI): 311 m/z [M+H]⁺,

### Intermediate 11: 2-Fluoro-5-((6-fluoro-4-(3-hydroxypropyl)-1H-indol-5-yl)oxy)benzonitrile

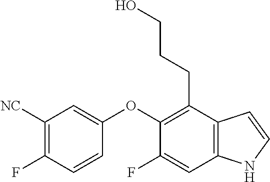 [SMILES: N#Cc1cc(Oc2c(F)cc3[nH]ccc3c2CCCO)ccc1F]

To a stirred and cooled (0° C.) solution of 5-((4-allyl-6-fluoro-1H-indol-5-yl)oxy)-2-fluoro-benzonitrile (Intermediate 10, 3.7 g, 12 mmol) in dry tetrahydrofuran (60 ml) was added borane-dimethyl sulfide complex (2 M in tetrahydrofuran, 12 mL, 24 mmol). The mixture was stirred at the same temperature for 2 hours, then treated with sodium acetate (6 N in water, 12 mL) followed by 30% of hydrogen peroxide (5 mL) and stirred for another 2 hours after the addition. The mixture was diluted with ethyl acetate (200 ml). The solution was washed with brine, dried over sodium sulfate, and concentrated. The residue was purified by automated flash chromatography (40 g silica gel column, eluting with 0-50% ethyl acetate in petroleum ether) to give the title compound (2.04 g, 52%) as a solid. MS (ESI): 329 m/z [M+H]⁺, retention time: 1.86 minutes, purity: 93% (214 nm) (LC-MS method 2). ¹H NMR (400 MHz, CDCl₃) δ 8.36 (s, 1H), 7.30-7.26 (m, 1H), 7.22-7.08 (m, 3H), 7.00 (dd, J=4.8, 3.1 Hz, 1H), 6.62 (dd, J=3.8, 1.5 Hz, 1H), 3.63 (d, J=3.4 Hz, 2H), 2.96-2.85 (m, 2H), 1.95-1.83 (m, 2H), 1.47 (s, 1H) ppm.

### Intermediate 12: Methyl 7,7-difluoro-2-(3-iodophenyl)-2-methylnon-8-ynoate

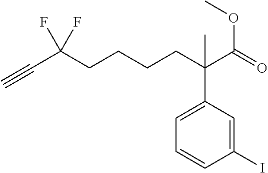 [SMILES: C#CC(F)(F)CCCCC(C)(C(=O)OC)c1cccc(I)c1]

To a stirred solution of methyl 2-(3-iodophenyl)-2-methyl-7-oxonon-8-ynoate (Intermediate 12E, 2.0 g, 4.5 mmol) in [C8 mim][PF6] (10 mL) was added diethylaminosulfur trifluoride (3.2 mL, 22.9 mmol). The mixture was stirred at 50° C. for four hours, then diluted with ethyl acetate (100 mL). The solution was washed with water, brine, dried over sodium sulfate, and concentrated. The residue was purified by automated flash chromatography (40 g silica gel column, eluting with 0-30% ethyl acetate in petroleum) to give the title compound (840 mg, 41.1%) as colorless oil. MS (ESI): 443 m/z [M+Na]⁺, retention time: 2.24 minutes, purity: 90% (254 nm) (LC-MS method 5).

The following intermediates were prepared utilizing the procedures described for Intermediate 12 and/or for Intermediate 12A to 12D.

| Inter. | | | MS m/z |
|---|---|---|---|
| No. | Structure | Name | [M + H]⁺ |
| 12-1 | 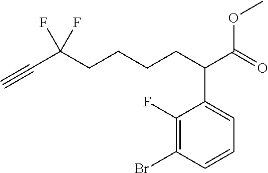 [SMILES: C#CC(F)(F)CCCCC(C(=O)OC)c1cccc(Br)c1F] | methyl 2-(3-bromo-2- fluorophenyl)-7,7-difluoronon-8- ynoate | 399, 401 [M + Na]⁺ |
| 12-2 | 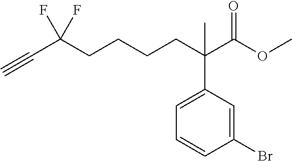 [SMILES: C#CC(F)(F)CCCCC(C)(C(=O)OC)c1cccc(Br)c1] | methyl 2-(3-bromophenyl)- 7,7-difluoro-2-methylnon-8- ynoate | 395, 397 [M + Na]⁺ |

### Intermediate 12A: Methyl 7-hydroxy-2-(3-iodophenyl)-2-methylheptanoate

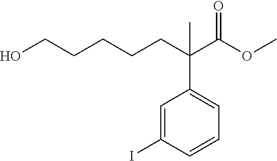 [SMILES: COC(=O)C(C)(CCCCCO)c1cccc(I)c1]

To a stirred solution of methyl 7-((tert-butyldimethylsilyl)oxy)-2-(3-iodophenyl)-2-methylheptanoate (Intermediate 7A-1, 11.8 g, 21.7 mmol) in tetrahydrofuran (25 mL) was added tetrabutylammonium fluoride (21.7 mL, 21.7 mmol, 1 M in tetrahydrofuran). The reaction was stirred at room temperature for 2 hours, then diluted with ethyl acetate (100 mL). The mixture was washed with water, dried over sodium sulfate, and concentrated. The residue was purified by automated flash chromatography (40 g silica gel column, eluting with 0-20% ethyl acetate in petroleum ether) to give the title compound (7 g, 81.6%) as white oil. MS (ESI): 377 m/z [M+H]⁺, retention time: 2.09 minutes, purity: 95% (254 nm) (LC-MS method 5).

The following intermediate was prepared utilizing the procedures described for Intermediate 12A.

| Inter. | | | MS m/z |
|---|---|---|---|
| No. | Structure | Name | [M + H]⁺ |
| 12A-1 | 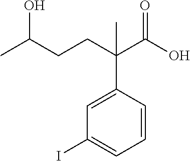 [SMILES: CC(O)CCC(C)(C(=O)O)c1cccc(I)c1] | 5-hydroxy-2-(3-iodophenyl)- 2-methylhexanoic acid | 371 [M + Na]⁺ |
| 12A-2 | 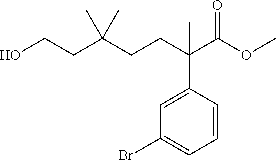 [SMILES: COC(=O)C(C)(CCC(C)(C)CCO)c1cccc(Br)c1] | methyl 2-(3-bromophenyl)- 7-hydroxy-2,5,5- trimethylheptanoate | 357, 359 |
| 12A-3 | 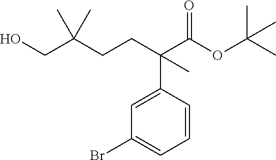 [SMILES: CC(C)(CO)CCC(C)(C(=O)OC(C)(C)C)c1cccc(Br)c1] | tert-butyl 2-(3-bromophenyl)- 6-hydroxy-2,5,5- trimethylhexanoate | 407, 409 [M + Na]⁺ |
| 12A-4 | 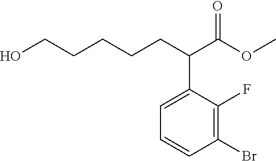 [SMILES: COC(=O)C(CCCCCO)c1cccc(Br)c1F] | methy 2-(3-bromo-2-fluorophenyl)- 7-hydroxyheptanoate | 333, 335 |

### Intermediate 12B: Methyl 2-(3-iodophenyl)-2-methyl-7-oxoheptanoate

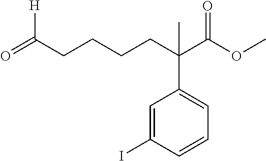 [SMILES: COC(=O)C(C)(CCCCC=O)c1cccc(I)c1]

To a stirred solution of methyl 7-hydroxy-2-(3-iodophenyl)-2-methylheptanoate (Intermediate 12A, 7 g, 17.7 mmol) in dichloromethane (100 mL) was added Dess-Martin periodinane (9 g, 21.2 mmol). The mixture was stirred at room temperature for 2 hours, quenched with water (100 mL), and extracted with dichloromethane (2×100 mL). The combined organic extracts were washed with saturated sodium bicarbonate, dried with sodium sulfate, and concentrated. The residue was purified by automated flash chromatography (40 g silica gel column, eluting with 0-20% ethyl acetate in petroleum ether) to give the title compound (4.5 g, 64.6%) as white oil. MS (ESI): 375 m/z [M+H]⁺, retention time: 2.14 minutes, purity: 95% (254 nm) (LC-MS method 5).

The following intermediate was prepared utilizing the procedures described for Intermediate 12B.

| Inter. | | | MS m/z |
|---|---|---|---|
| No. | Structure | Name | [M + H]⁺ |
| 12B-1 | 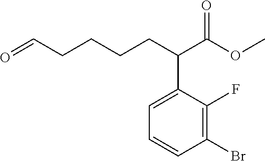 [SMILES: COC(=O)C(CCCCC=O)c1cccc(Br)c1F] | methyl 2-(3-bromo-2- fluorophenyl)-7-oxoheptanoate | 353, 55 [M + Na]⁺ |

### Intermediate 12C: Methyl 7-hydroxy-2-(3-iodophenyl)-2-methyl-9-(trimethylsilyl)non-8-ynoate

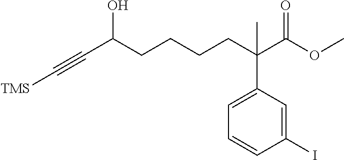 [SMILES: COC(=O)C(C)(CCCCC(O)C#C[Si](C)(C)C)c1cccc(I)c1]

To a stirred and cooled (−78° C.) solution of trimethylsilylacetylene (1.31 g, 13.4 mmol) in tetrahydrofuran (25 mL) was added n-butyl lithium (5.56 mL, 13.9 mmol, 2.5M in hexanes). The mixture was stirred at this temperature for one hour, then treated with a solution of methyl 2-(3-iodophenyl)-2-methyl-7-oxo-heptanoate (Intermediate 12B, 4 g, 10.7 mmol) in tetrahydrofuran (25 mL). After stirring for another hour at −78° C., the mixture was quenched with water, and extracted with ethyl acetate (3×50 mL). The combined organic layers were washed with brine, dried over sodium sulfate, and concentrated. The residue was purified by automated flash chromatography (10 g silica gel column, eluting with 0-20% ethyl acetate in petroleum) to give the title compound (3.9 g, 70%) as white oil. MS (ESI): 495 m/z [M+Na]⁺, retention time: 2.33 minutes, purity: 90% (254 nm) (LC-MS method 5).

The following intermediate was prepared utilizing the procedures described for Intermediate 12C.

| Inter. | | | MS m/z |
|---|---|---|---|
| No. | Structure | Name | [M + H]⁺ |
| 12C-1 | 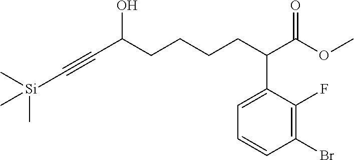 | methyl 2-(3-bromo-2- fluorophenyl)-7-hydroxy-9- (trimethylsilyl)non-8-ynoate | 451, 453 [M + Na]⁺ |

### Intermediate 12D: Methyl 7-hydroxy-2-(3-iodophenyl)-2-methylnon-8-ynoate

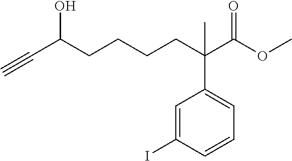 [SMILES: C#CC(O)CCCCC(C)(C(=O)OC)c1cccc(I)c1]

To a stirred solution of methyl 7-hydroxy-2-(3-iodophenyl)-2-methyl-9-(trimethylsilyl)non-8-ynoate (Intermediate 12C, 9.1 g, 19.2 mmol) in methanol (100 mL) and water (1 mL) was added potassium carbonate (3.99 g, 28.8 mmol). The mixture was stirred at room temperature for one hour and concentrated to remove methanol. The aqueous reside was diluted with ethyl acetate (150 mL), washed with water, dried over sodium sulfate, and concentrated. The crude product was purified by automated flash chromatography (120 g silica gel column, eluting with 0-30% ethyl acetate in petroleum) to give methyl 7-hydroxy-2-(3-iodophenyl)-2-methylnon-8-ynoate (6.4 g, 84%) as white oil. MS (ESI): 423 m/z [M+Na]⁺, retention time: 2.07 minutes, purity: 98% (254 nm) (LC-MS method 5).

### Intermediate 12E: Methyl 2-(3-iodophenyl)-2-methyl-7-oxonon-8-ynoate

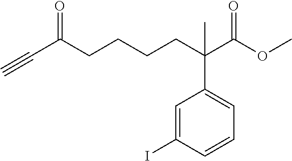 [SMILES: C#CC(=O)CCCCC(C)(C(=O)OC)c1cccc(I)c1]

To a stirred solution of methyl 7-hydroxy-2-(3-iodophenyl)-2-methylnon-8-ynoate (Intermediate 12D, 6.4 g, 16.0 mmol) in dichloromethane (100 mL) was added Dess-Martin periodinane (8.1 g, 19.2 mmol). The mixture was stirred at room temperature for 2 hours, quenched with saturated aqueous sodium bicarbonate (2×100 mL) and extracted with dichloromethane (2×100 mL). The combined dichloromethane layers were washed with brine, dried over sodium sulfate, and concentrated. The residue was purified by automated flash chromatography (80 g silica gel column, eluting with 0-20% ethyl acetate in petroleum ether) to give the title compound (4.1 g, 65%) as white oil. MS (ESI): 421 m/z [M+Na]⁺, retention time: 2.17 minutes, purity: 97% (254 nm) (LC-MS method 5).

### Intermediate 13: 7-Acetoxy-2-(3-bromo-2-fluorophenyl)non-8-ynoic acid

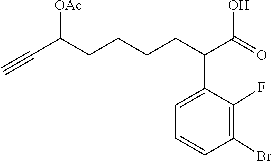 [SMILES: C#CC(CCCCC(C(=O)O)c1cccc(Br)c1F)OC(C)=O]

To a stirred solution of 2-(3-bromo-2-fluorophenyl)-7-hydroxynon-8-ynoic acid (Intermediate 13A, 2.5 g, 7.2 mmol) in acetic anhydride (10 mL) was added p-toluenesulfonic acid (137 mg, 0.72 mmol). The solution was stirred at room temperature for 1 hour, then treated with tetrahydrofuran (10 mL) and water (10 mL) and stirred overnight. The mixture was partitioned between brine (100 mL) and ethyl acetate (100 mL). The separated organic phase, combined with one additional ethyl acetate extract, was dried over magnesium sulfate, and concentrated. The residue was purified by flash chromatography (40 g silica gel column, eluting with 0-30% ethyl acetate in petroleum ether) to give the title compound (2.1 g, 75%) as oil. MS (ESI): 407, 409 m/z [M+Na]⁺, retention time: 1.98 minutes, purity: 90% (254 nm) (LC-MS method 5).

### Intermediate 13A: 2-(3-Bromo-2-fluorophenyl)-7-hydroxynon-8-ynoic acid

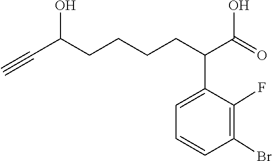 [SMILES: C#CC(O)CCCCC(C(=O)O)c1cccc(Br)c1F]

To a stirred solution of methyl 2-(3-bromo-2-fluorophenyl)-7-hydroxy-9-(trimethylsilyl)non-8-ynoate (Intermediate 12C-1, 3.5 g, 8.1 mmol) in tetrahydrofuran (30 mL) and water (8 mL) was added lithium hydroxide (1.6 g, 40.7 mmol). The reaction was stirred at 60° C. for 16 hours, cooled to 0° C. and acidified with 1 M hydrochloric acid to pH ˜4. The mixture was extracted with ethyl acetate (2×100 mL). The combined organic extracts were washed with brine, dried over sodium sulfate, and concentrated. The residue was purified by flash chromatography (12 g silica gel column, eluting with 5% methanol in dichloromethane) to give the title compound (2.5 g, 90%) as oil. MS (ESI): 365, 367 m/z [M+Na]⁺, retention time: 1.82 minutes, purity: 90% (254 nm) (LC-MS method 5).

### Intermediate 14: Methyl 7-azido-2-(3-iodophenyl)-2-methylheptanoate

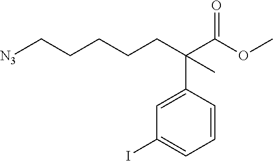 [SMILES: COC(=O)C(C)(CCCCCN=[N+]=[N-])c1cccc(I)c1]

To a stirred solution of methyl 7-chloro-2-(3-iodophenyl)-2-methylheptanoate (Intermediate 7A-2, 3.3 g, 8.37 mmol) in dimethylformamide (50 mL) was added sodium azide (3.26 g, 50.2 mmol) and potassium iodide (2.78 g, 16.7 mmol). The reaction was heated at 80° C. overnight and concentrated. The residue was dissolved in ethyl acetate, washed with water, brine, dried over sodium sulfate, and concentrated. The crude product was purified by automated silica gel column chromatography (eluting with petroleum ether/ethyl acetate=0-30%) to give the title compound (2.8 g, 85%) as an oil. MS (ESI): 424 m/z [M+Na]⁺, retention time: 2.30 minutes, purity: 92% (254 nm) (LC-MS method 5). ¹H NMR (400 MHz, CDCl₃) δ7.62-7.56 (m, 2H), 7.26-7.24 (m, 1H), 7.07-7.03 (m, 1H), 3.66 (s, 3H), 3.25-3.21 (m, 2H), 2.04-1.85 (m, 2H), 1.61-1.54 (m, 2H), 1.517 (s, 3H), 1.41-1.35 (m, 2H), 1.25-1.16 (m, 2H) ppm.

### Intermediate 15: 2-Fluoro-5-((6-fluoro-1-tosyl-4-vinyl-1H-indol-5-yl)oxy)benzonitrile

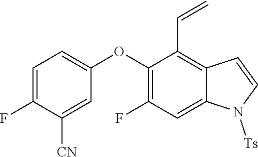 [SMILES: C=Cc1c(Oc2ccc(F)c(C#N)c2)c(F)cc2c1ccn2S(=O)(=O)c1ccc(C)cc1]

To a suspension of 5-((4-bromo-6-fluoro-1-tosyl-1H-indol-5-yl)oxy)-2-fluorobenzonitrile (Intermediate 4, 5.25 g, 10.5 mmol) in dioxane (120 mL) and water (40 mL) were added 4,4,5,5-tetramethyl-2-vinyl-1,3,2-dioxaborolane (3.2 g, 21 mmol), Pd(dppf)Cl₂ (384 mg, 0.5 mmol) and cesium carbonate (6.8 g, 21 mmol). The reaction mixture was stirred at 100° C. overnight, cooled to room temperature and extracted with ethyl acetate(150 mL×3). The combined organic extracts were washed with brine, dried over magnesium sulfate, and evaporated to dryness. The resulting residue was purified by flash chromatography over silica (petroleum ether/dichloromethane, v/v, 2/1) to afford the title compound as a yellow solid (4 g, 85%). MS: 473 m/z [M+Na]⁺.

The following intermediate was prepared utilizing the procedures described for Intermediate 15.

| Inter. | | | MS m/z |
|---|---|---|---|
| No. | Structure | Name | [M + H]⁺ |
| 15-1 | 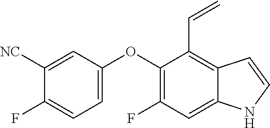 [SMILES: C=Cc1c(Oc2ccc(F)c(C#N)c2)c(F)cc2[nH]ccc12] | 2-fluoro-5-((6-fluoro- 4-vinyl-1H-indol-5- yl)oxy)benzonitrile | 297 |
| 15-2 | 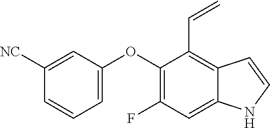 [SMILES: C=Cc1c(Oc2cccc(C#N)c2)c(F)cc2[nH]ccc12] | 3-((6-fluoro-4-vinyl-1H- indol-5-yl)oxy)benzonitrile | 279 |
| 15-3 | 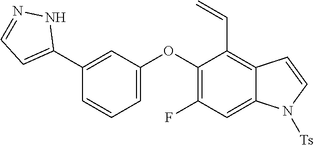 [SMILES: C=Cc1c(Oc2cccc(-c3ccn[nH]3)c2)c(F)cc2c1ccn2S(=O)(=O)c1ccc(C)cc1] | 5-(3-(1H-pyrazol-5-yl)phenoxy)- 6-fluoro-1-tosyl-4- vinyl-1H-indole | 474 |
| 15-4 | 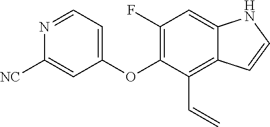 [SMILES: C=Cc1c(Oc2ccnc(C#N)c2)c(F)cc2[nH]ccc12] | 4-((6-fluoro-4-vinyl-1H-indol- 5-yl)oxy)picolinonitrile | 280 |
| 15-5 | 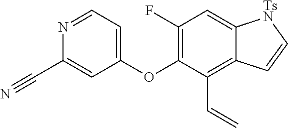 [SMILES: C=Cc1c(Oc2ccnc(C#N)c2)c(F)cc2c1ccn2S(=O)(=O)c1ccc(C)cc1] | 4-((6-fluoro-1-tosyl-4-vinyl-1H-indol-5- yl)oxy)picolinonitrile | 434 |
| 15-6 | 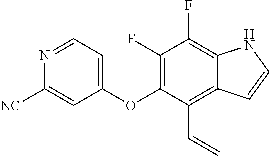 [SMILES: C=Cc1c(Oc2ccnc(C#N)c2)c(F)c(F)c2[nH]ccc12] | 4-((6,7-difluoro-4-vinyl-1H-indol-5- yl)oxy)picolinonitrile | 298 |

### Intermediate 16: 2-Fluoro-5-((6-fluoro-4-formyl-1-tosyl-1H-indol-5-yl)oxy)benzonitrile

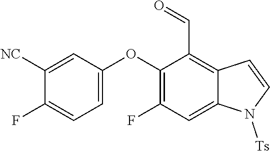 [SMILES: Cc1ccc(S(=O)(=O)n2ccc3c(C=O)c(Oc4ccc(F)c(C#N)c4)c(F)cc32)cc1]

To a solution of 2-fluoro-5-((6-fluoro-1l-tosyl-4-vinyl-1H-indol-5-yl)oxy)benzonitrile (Intermediate 15, 4.6 g, 10.2 mmol) in tetrahydrofuran (90 mL) were added 2,6-lutidine (1.1 g, 10.2 mmol) and osmium tetroxide (2 mL saturated in water) at 0° C. The reaction mixture was stirred for three minutes and a solution of sodium periodate (8.8 g, 4.0 mmol) in water (30 mL) was added. The reaction mixture was stirred at room temperature overnight, acidified with 2 M hydrochloric acid (100 mL) and extracted with ethyl acetate (200 ml×3). The combined organic extracts were washed with water, brine, dried over magnesium sulfate and evaporated to dryness. The resulting residue was purified by flash chromatography over silica (petroleum ether/ethyl acetate, v/v, 10/1) to afford the title compound as a yellow solid (3.5 g, 75%). MS: 453 m/z [M+H]⁺.

The following intermediate was prepared utilizing the procedures described for Intermediate 16.

| Inter. | | | MS m/z |
|---|---|---|---|
| No. | Structure | Name | [M + H]⁺ |
| 16-1 | 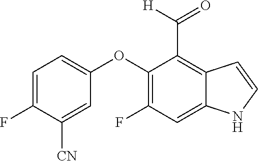 [SMILES: N#Cc1cc(Oc2c(F)cc3[nH]ccc3c2C=O)ccc1F] | 2-fluoro-5-((6-fluoro-4-formyl-1H-indol-5- yl)oxy)benzonitrile | 299 |
| 16-2 | 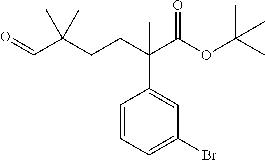 [SMILES: CC(C)(C=O)CCC(C)(C(=O)OC(C)(C)C)c1cccc(Br)c1] | tert-butyl 2-(3-bromophenyl)-2,5,5-trimethyl-6- oxohexanoate | 405, 407 [M + Na]⁺ |
| 16-3 | 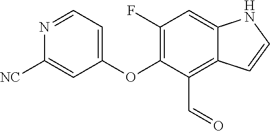 [SMILES: N#Cc1cc(Oc2c(F)cc3[nH]ccc3c2C=O)ccn1] | 4-((6-fluoro-4-formyl-1H-indol-5- yl)oxy)picolinonitrile | 282 |
| 16-4 | 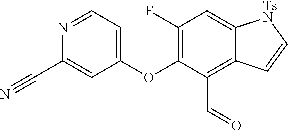 [SMILES: Cc1ccc(S(=O)(=O)n2ccc3c(C=O)c(Oc4ccnc(C#N)c4)c(F)cc32)cc1] | 4-((6-fluoro-4-formyl-1-tosyl-1H-indol-5- yl)oxy)picolinonitrile | 436 |
| 16-5 | 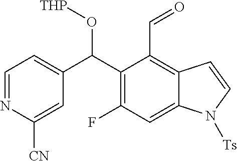 | 4-((6-fluoro-4-formyl-1-tosyl-1H-indol-5- yl)((tetrahydro-2H-pyran-2- yl)oxy)methyl)picolinonitrile | 534 m/z [M + H]⁺; RT: 2.19 + 2.23 minutes (LC- MS method 4) |
| 16-6 | 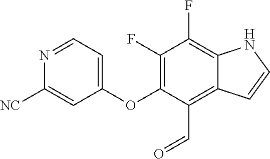 [SMILES: N#Cc1cc(Oc2c(F)c(F)c3[nH]ccc3c2C=O)ccn1] | 4-((6,7-difluoro-4-formyl-1H-indol-5- yl)oxy)picolinonitrile | 300 m/z [M + H]⁺; RT: 1.55 minutes (LC-MS method 2) |

### Intermediate 17: 2-Fluoro-5-((6-fluoro-4-(1-hydroxyprop-2-yn-1-yl)-1H-indol-5-yl)oxy)-benzonitrile

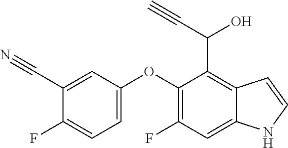 [SMILES: C#CC(O)c1c(Oc2ccc(F)c(C#N)c2)c(F)cc2[nH]ccc12]

To a stirred solution of the mixture of Intermediate 17A (4.5 g, 8.2 mmol) in methanol (90 mL) was added potassium carbonate (3.38 g, 24.6 mmol). The reaction was heated at 60° C. for two hours, cooled to room temperature and partitioned between water (100 mL) and ethyl acetate (100 mL). The separated organic layer, combined with two additional ethyl acetate extracts (2×50 mL), was washed with brine, dried over sodium sulfate, and concentrated. The residue was purified by flash chromatography (40 g silica gel column, eluting with 0-40% ethyl acetate in petroleum ether) to give the title compound (1.8 g, 70%) as a solid. MS (ESI): 347 m/z [M+Na]⁺, retention time: 1.84 minutes, purity: 91% (214 nm) (LC-MS method 2).

Intermediate 17A: Mixture of 2-fluoro-5-((6-fluoro-4-(1-hydroxy-3-(trimethylsilyl)prop-2-yn-1-yl)-1-tosyl-1H-indol-5-yl)oxy)benzonitrile and 2-fluoro-5-((6-fluoro-4-(1-hydroxyprop-2-yn-1-yl)-1-tosyl-1H-indol-5-yl)oxy)benzonitrile

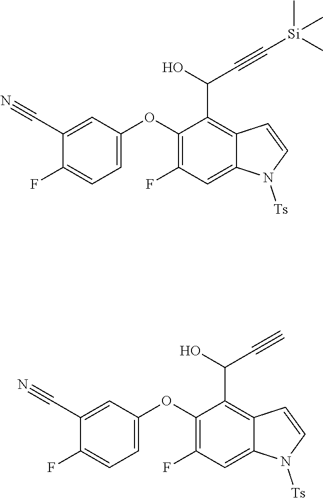 [SMILES: Cc1ccc(S(=O)(=O)n2ccc3c(C(O)C#C[Si](C)(C)C)c(Oc4ccc(F)c(C#N)c4)c(F)cc32)cc1 ; C#CC(O)c1c(Oc2ccc(F)c(C#N)c2)c(F)cc2c1ccn2S(=O)(=O)c1ccc(C)cc1]

To a stirred and cooled (−78° C.) solution of trimethylsilylacetylene (10.9 g, 111 mmol) in tetrahydrofuran (300 mL) was added n-butyl lithium (44 mL, 111 mmol, 2.5 M in hexanes) slowly. The reaction was stirred at −78° C. for 15 minutes, then treated with hexamethylphosphoramide (19.4 mL, 111 mmol) was added at this temperature. After the addition, the reaction mixture was stirred at 0° C. for 45 minutes and re-cooled to −78° C. and added a solution of 2-fluoro-5-((6-fluoro-4-formyl-1-tosyl-1H-indol-5-yl)oxy)benzonitrile (Intermediate 16, 5 g, 11.1 mmol) in tetrahydrofuran (100 mL). The mixture was stirred at −78° C. for 2 hours, quenched with water (100 mL). After warming to room temperature, the mixture was extracted with ethyl acetate (3×150 mL). The combined organic phases were washed with brine, dried over sodium sulfate, and concentrated. The residue was purified by silica gel column chromatography (eluting with petroleum ether/ethyl acetate=0-40%) to give the mixture of title compounds (4.5 g, 75%). For TMS protected product: MS (ESI): 573 m/z [M+H]⁺, retention time: 2.25 minutes, purity: 51% (214 nm); For TMS deprotected product: MS (ESI): 501 m/z [M+H]⁺, retention time: 2.09 minutes, purity: 29% (214 nm) (LC-MS method 5).

Intermediate 18: 2-(3-Iodophenyl)-2-methyl-5-(prop-2-yn-1-yloxy)hexanoic acid

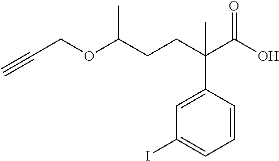 [SMILES: C#CCOC(C)CCC(C)(C(=O)O)c1cccc(I)c1]

To a stirred and cooled (−78° C.) solution of 5-hydroxy-2-(3-iodophenyl)-2-methylhexanoic acid (Intermediate 12A-1, 4 g, 11.5 mmol) in tetrahydrofuran (40 mL) was added sodium bis(trimethylsilyl)amide (2 M in THF, 14.4 mL, 28.7 mmol). The reaction was stirred at this temperature for 0.5 hours, then treated with 3-bromoprop-1-yne (2.05 g, 17.2 mmol). The mixture was allowed to warm to room temperature and stirred for another 16 hours, quenched with saturated ammonium chloride solution (100 mL). The mixture was extracted with ethyl acetate (3×50 mL). The combined organic extracts were washed with brine (50 mL), dried over sodium sulfate, and concentrated. The residue was purified by column chromatography on silica gel using ethyl acetate/petroleum ether (20:80) to afford the title compound (1.5 g, 34%) as an oil. MS (ESI): 409 m/z [M+Na]⁺, retention time: 1.31 minutes, purity: 67% (254 nm) (LC-MS method 8).

Intermediate 19: 2-(3-Iodophenyl)-2,5-dimethylnon-8-ynoic acid

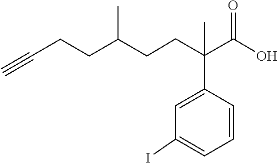 [SMILES: C#CCCC(C)CCC(C)(C(=O)O)c1cccc(I)c1]

To a stirred solution of 2-(3-iodophenyl)-2,5-dimethyl-9-(trimethylsilyl)non-8-ynoic acid (Intermediate 19A, 4.5 g, crude) in 200 mL of methanol was added potassium carbonate (5.52 g, 40 mmol). The reaction was stirred for two hours and concentrated. The residue was acidified with 6 M hydrochloric acid (20 mL) and water (30 mL), then extracted with ethyl acetate (3×100 mL). The combined organic phases were washed with brine, dried over sodium sulfate, and concentrated. The crude product was purified by silica gel chromatography to afford the title compound (3.23 g, 84% for two step). MS (ESI): 407 m/z [M+Na]⁺, retention time: 2.55 minutes, purity: 64% (254 nm) (LC-MS method 5).

The following intermediate was prepared utilizing the procedures described for Intermediate 19 and/or for Intermediate 19A.

| Inter. | | | MS m/z |
|---|---|---|---|
| No. | Structure | Name | [M + H]⁺ |
| 19-1 | 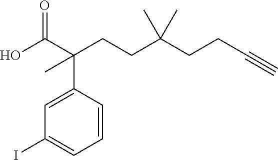 [SMILES: C#CCCC(C)(C)CCC(C)(C(=O)O)c1cccc(I)c1] | 2-(3-iodophenyl)-2,5,5-trimethylnon-8-ylnoic acid | 399 |
| 19-2 | 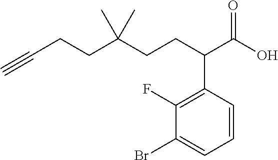 [SMILES: C#CCCC(C)(C)CCC(C(=O)O)c1cccc(Br)c1F] | 2-(3-bromo-2-fluorophenyl)-5,5-dimethylnon-8- ynoic acid | 377, 379 [M + Na]⁺ |
| 19-3 | 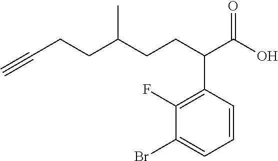 [SMILES: C#CCCC(C)CCC(C(=O)O)c1cccc(Br)c1F] | 2-(3-bromo-2-fluorophenyl)-5-methylnon-8-ynoic acid | 363, 365 [M + Na]⁺ |

Intermediate 19A: 2-(3-Iodophenyl)-2,5-dimethyl-9-(trimethylsilyl)non-8-ynoic acid

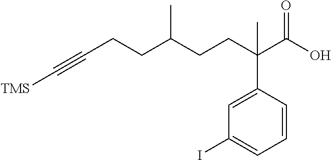 [SMILES: CC(CCC#C[Si](C)(C)C)CCC(C)(C(=O)O)c1cccc(I)c1]

To a stirred and cooled (−78° C.) solution of trimethylsilylacetylene (3.92 g, 40 mmol) in tetrahydrofuran (50 mL) was added n-butyl lithium (2.5 M in hexane, 16 mL, 40 mmol) dropwise. The reaction was stirred at this temperature for 0.5 hours, then treated with hexamethylphosphoramide (7.16 g, 40 mmol) and stirred for another 0.5 hours, followed by addition of 7-bromo-2-(3-iodophenyl)-2,5-dimethylheptanoic acid (Intermediate 7-5, 4.39 g, 10 mmol). The mixture was allowed to warm to room temperature and stirred overnight, quenched with saturated ammonium chloride (100 mL), and extracted with ethyl acetate (3×100 mL). The combined organic layers were washed with brine, dried over sodium sulfate, and concentrated to give the crude title compound (4.5 g). This crude compound was used for next step without further purification. MS (ESI): 479 m/z [M+Na]⁺, retention time: 2.55 minutes, purity: 64% (254 nm) (LC-MS method 5).

Intermediate 20: 6-Acetoxy-7-azido-2-(3-iodophenyl)-2-methylheptanoic acid

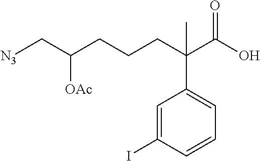 [SMILES: CC(=O)OC(CCCC(C)(C(=O)O)c1cccc(I)c1)CN=[N+]=[N-]]

To the stirred solution of 7-azido-6-hydroxy-2-(3-iodophenyl)-2-methylheptanoic acid (Intermediate 20D, 1 g, 2.6 mmol) in acetic anhydride (5 mL) was added p-toluenesulfonic acid (30 mg, 0.2 mmol). The reaction was stirred for 4 hours treated with water/tetrahydrofuran (100 mL, 10/1) and stirred for another 1 hour. The mixture was concentrated. The residue was partitioned between water (50 mL) and ethyl acetate (100 mL). The separated organic layer was washed with brine, dried over magnesium sulfate and concentrated. The crude product was purified by automated flash chromatography (eluting with 30% ethyl acetate/petroleum ether) to give the title compound (900 mg, 70%) as a white solid. MS (ESI): 468 m/z [M+Na]⁺, retention time: 1.56 minutes, purity: 95% (254 nm) (LC-MS method 2).

The following intermediate was prepared utilizing the procedures described for Intermediate 16.

| Inter. | | | MS m/z |
|---|---|---|---|
| No. | Structure | Name | [M + H]⁺ |
| 20-1 | 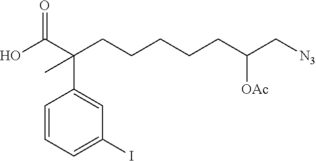 [SMILES: CC(=O)OC(CCCCCC(C)(C(=O)O)c1cccc(I)c1)CN=[N+]=[N-]] | 8-acetoxy-9-azido-2-(3-iodophenyl)-2- methylnonanoic acid | 474 |

Intermediate 20A: Methyl 6,7-dihydroxy-2-(3-iodophenyl)-2-methylheptanoate

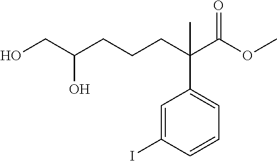 [SMILES: COC(=O)C(C)(CCCC(O)CO)c1cccc(I)c1]

To the stirred solution of methyl 2-(3-iodophenyl)-2-methylhept-6-enoate (Intermediate 7A-5, 10 g, 28 mmol) and N-methylmorpholine N-oxide (4.9 g, 42 mmol) in 100 mL of tetrahydrofuran was added osmium tetroxide (100 mg, 4% in water) at room temperature and stirred for 24 hours and concentrated. The residue was partitioned between water (150 mL) and ethyl acetate (150 mL). The separated organic layer, combined with two additional ethyl acetate (2×150 mL) extracts, was washed with brine, dried over magnesium sulfate, and concentrated. The crude product was purified by automated flash chromatography (80 g silica gel column, eluting with 30% ethyl acetate/petroleum ether) to give the title compound (9 g, 85%) as a white solid. MS (ESI): 393 m/z [M+H]⁺, retention time: 1.56 minutes, purity: 95% (214 nm) (LC-MS method 2).

Intermediate 20B: Ethyl 6-hydroxy-2-(3-iodophenyl)-2-methyl-7-((methylsulfonyl)oxy)-heptanoate

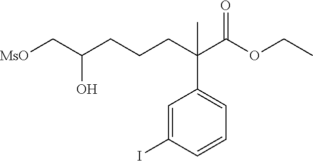 [SMILES: CCOC(=O)C(C)(CCCC(O)COS(C)(=O)=O)c1cccc(I)c1]

To a stirred solution of ethyl 6,7-dihydroxy-2-(3-iodophenyl)-2-methylheptanoate (3.5 g, 8.62 mmol) and triethylamine (1.74 g, 17.2 mmol) in 200 mL of dichloromethane was added methanesulfonyl chloride (879 mg, 8.62 mmol) dropwise over 5 minutes at room temperature.

The mixture was stirred for 16 hours, then quenched with ice-water (100 mL). The separated organic layer was washed with saturated potassium carbonate, brine, dried over sodium sulfate, and concentrated to give the crude title compound (3.9 g, 98%) as a yellow oil. The crude product was used for the next step without further purification. MS (ESI): 485 m/z [M+H]⁺, retention time: 1.91 minutes, purity: 78% (254 nm) (LC-MS method 2).

Intermediate 20C: Ethyl 7-azido-6-hydroxy-2-(3-iodophenyl)-2-methylheptanoate

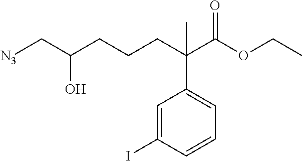 [SMILES: CCOC(=O)C(C)(CCCC(O)CN=[N+]=[N-])c1cccc(I)c1]

To a stirred solution of ethyl 6-hydroxy-2-(3-iodophenyl)-2-methyl-7-((methylsulfonyl)oxy)-heptanoate (Intermediate 20B, 500 mg, 1.05 mmol) in 5 mL of dimethylformamide was added sodium azide (137 mg, 2.10 mmol). The mixture was stirred at 80° C. for 8 hours, quenched with ice-water (20 mL), and diluted with ethyl acetate (50 mL). The separated organic layer was washed with brine, dried over sodium sulfate, and concentrated. The residue was purified by automated flash chromatography (eluting with petroleum ether/ethyl acetate=1/1) to give the title compound (0.4 g, 75%) as a yellow oil. MS (ESI): 432 m/z [M+H]⁺, retention time: 1.94 minutes, purity: 78% (254 nm) (LC-MS method 2).

Intermediate 20D: 7-Azido-6-hydroxy-2-(3-iodophenyl)-2-methylheptanoic acid

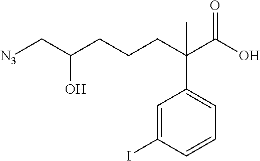 [SMILES: CC(CCCC(O)CN=[N+]=[N-])(C(=O)O)c1cccc(I)c1]

To a stirred solution of ethyl 7-azido-6-hydroxy-2-(3-iodophenyl)-2-methylheptanoate (Intermediate 20C, 1 g, 2.35 mmol) in tetrahydrofuran (10 mL) was added lithium hydroxide (1.88 g, 47.1 mmol). The reaction was stirred for 18 hours, then acidified to pH ˜4 with 1 N hydrochloric acid. The mixture was extracted with ethyl acetate (2×50 mL). The combined organic phases were dried over magnesium sulfate and concentrated to provide the title compound (0.8 g, 90%) as brown oil. MS (ESI): 404 m/z [M+H]⁺, retention time: 1.78 minutes, purity: 96% (254 nm) (LC-MS method 2).

Intermediate 21: Methyl (E)-3-(5-(3-cyano-4-fluorophenoxy)-6-fluoro-1H-indol-4-yl)-acrylate

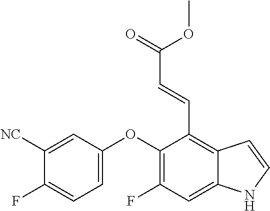 [SMILES: COC(=O)/C=C/c1c(Oc2ccc(F)c(C#N)c2)c(F)cc2[nH]ccc12]

To a stirred and degassed solution of 5-((4-bromo-6-fluoro-1H-indol-5-yl)oxy)-2-fluoro-benzonitrile (Intermediate 3, 15 g, 4.31 mmol) in 100 mL of N,N′-dimethylformamide was added methyl acrylate (4.08 g, 4.74 mmol), tri-o-tolyl phosphine (2.62 g, 0.86 mmol), triethylamine (13.05 g, 12.9 mmol) and palladium (II) acetate (1 g, 0.04 mmol). The reaction was stirred at 120° C. for 18 hours under nitrogen, cooled to room temperature and partitioned between water (200 mL) and ethyl acetate (200 mL). The separated organic layer, combined with two additional ethyl acetate extracts (2×200 mL), was washed with 1 N hydrochloric acid, brine, dried over magnesium sulfate, and concentrated. The residue was purified by automated flash chromatography (80 g silica gel column, eluting with 90% ethyl acetate/hexane) to provide the title compound (9 g, 60%) as a yellow oil. MS (ESI): 355 m/z [M+H]⁺, retention time: 1.82 minutes, purity: 94% (214 nm) (LC-MS method 2).

Intermediate 22: 2-(But-3-yn-1-yl)-2-(2-iodoethyl)-1,3-dioxolane

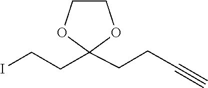

To a stirred and cooled (0° C.) solution of imidazole (8.3 g, 122.35 mmol) and triphenylphosphine (32.1 g, 122.35 mmol) in dichloromethane (50 mL) was added iodine (31.1 g, 122.35 mmol). The solution was stirred for 5 minutes, then slowly treated with a solution of 2-(2-(but-3-yn-1-yl)-1,3-dioxolan-2-yl)ethan-1-ol (16.0 g, 94.1 mmol) in dichloromethane (50 mL). The reaction mixture was stirred for 4 hours, quenched with aqueous saturated sodium thiosulfate (50 mL) and extracted with ethyl acetate (3×150 mL). The combined organic layers were washed with brine, dried over sodium sulfate, and concentrated. The residue was purified by flash column chromatography (petroleum ether/ethyl acetate=20:1) to yield the title compound (21.0 g, 80%) as colorless oil. ¹H NMR (400 MHz, CDCl₃) δ 3.95 (s, 4H), 3.16-3.12 (m, 2H), 2.30-2.24 (m, 4H), 1.95 (t, J=2.8 Hz, 1H), 1.89-1.85 (m, 2H) ppm.

Intermediate 23: 5-Acetoxy-2-(3-iodophenyl)-2-methylnon-8-ynoic acid

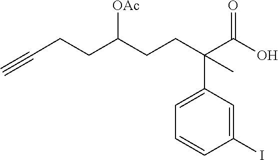 [SMILES: C#CCCC(CCC(C)(C(=O)O)c1cccc(I)c1)OC(C)=O]

A solution of 5-hydroxy-2-(3-iodophenyl)-2-methylnon-8-ynoic acid (Intermediate 23B, 6.3 g, 16.32 mmol) and p-toluene sulphonic acid (0.28 g, 1.63 mmol) in acetic anhydride (20 mL) was stirred at 40° C. for 16 hours, then treated with a mixture of 30 mL of tetrahydrofuran and 15 mL of water and stirred at 40° C. for additional 16 hours. The mixture was concentrated. The residue was partitioned between water (100 mL) and ethyl acetate (100 mL). The separated organic layer, combined with two additional ethyl acetate extracts (2×100 mL), was dried over sodium sulfate, and concentrated. The residue was purified by silica gel column chromatography (eluting with petroleum ether/ethyl acetate=3/1) to give the title compound (3.0 g, 43%) as a yellow oil. MS (ESI): 451 m/z [M+Na]⁺, retention time: 2.05 minutes, purity: 67% (214 nm) (LC-MS Method 9).

Intermediate 23A: 2-(3-Iodophenyl)-2-methyl-5-oxonon-8-ynoic acid

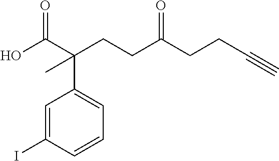 [SMILES: C#CCCC(=O)CCC(C)(C(=O)O)c1cccc(I)c1]

A solution of 4-(2-(but-3-yn-1-yl)-1,3-dioxolan-2-yl)-2-(3-iodophenyl)-2-methylbutanoic acid (Intermediate 7-7, 7.5 g, 17.52 mmol) and hydrochloric acid (3 M in water, 17.52 mL, 52.57 mmol) in tetrahydrofuran (20 mL) was stirred at 40° C. for 16 hours and concentrated. The residue was partitioned between water (100 mL) and ethyl acetate (150 mL). The separated organic layer, combined with two additional ethyl acetate extracts (2×150 mL), was dried over sodium sulfate, and concentrated to give the title compound (6.6 g, 98%) as a yellow oil. MS (ESI): 407 m/z [M+Na]⁺, retention time: 1.95 minutes, purity: 96% (214 nm) (LC-MS method 9).

Intermediate 23B: 5-Hydroxy-2-(3-iodophenyl)-2-methylnon-8-ynoic acid

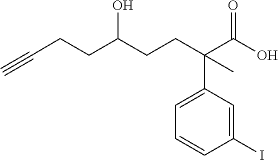 [SMILES: C#CCCC(O)CCC(C)(C(=O)O)c1cccc(I)c1]

To a stirred and cooled (0° C.) solution of 2-(3-iodophenyl)-2-methyl-5-oxonon-8-ynoic acid (Intermediate 23A, 6.6 g, 17.19 mmol) in methanol (50 mL) was added sodium borohydride (1.27 g, 34.38 mmol) portion wise. After the addition, the reaction was stirred for 30 minutes, then quenched with brine (30 mL). The aqueous phase was acidified with 1 N hydrochloric acid to pH ˜5 and then extracted with ethyl acetate (3×30 mL). The combined organic phase was dried over sodium sulfate and concentrated to give the title compound (6.3 g, 95%) as a yellow oil. MS (ESI): 409 m/z [M+Na]⁺, retention time: 1.88 minutes, purity: 69% (214 nm) (LC-MS Method 9).

Intermediate 24: 3-(3-Iodopropoxy)-3-methylbut-1-yne

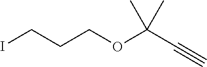

To a stirred solution and cooled (0° C.) of imidazole (3.26 g, 48 mmol), triphenylphosphine (12.6 g, 48 mmol) and iodine (10.2 g, 48 mmol) in dichloromethane (100 mL) was added 3-(1,1-dimethylprop-2-ynoxy)propan-1-ol (Intermediate 24B, 5.68 g, 40 mmol). The reaction was stirred for 3 hours at room temperature, then quenched with water (40 mL). The mixture was extracted with dichloromethane (3×60 mL). The combined organic phase was washed with brine (80 mL), dried over sodium sulfate, and concentrated. The residue was purified by automated flash chromatography (40 g silica gel column, eluting with petroleum ether/ethyl acetate=20/1) to give the title compound (7.2 g, 71%) as colorless oil. ¹H NMR (400 MHz, CDCl₃) δ 3.60 (t, J=5.6 Hz, 2H), 3.28 (t, J=6.8 Hz, 2H), 2.43 (s, 1H), 2.12-2.02 (m, 2H), 1.46 (s, 6H) ppm.

The following intermediate was prepared utilizing the procedures described for Intermediate 24.

| Inter. | | | MS m/z |
|---|---|---|---|
| No. | Structure | Name | [M + H]⁺ |
| 24-1 | 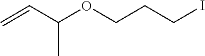 | 3-(3- iodopropoxy)but-1- ene | ¹H NMR (400 MHz, CDCl₃) δ 5.78-5.68 (m, 1H), 5.24-5.08 (m, 2H), 3.84-3.80 (m, 1H), 3.59-3.44 (m, 2H), 3.41-3.33 (m, 1H), 3.32-3.23 (m, 1H), 2.10- 1.98 (m, 2H), 1.23 (d, J = 6.4 Hz, 3H) ppm. |

Intermediate 24A: Ethyl 3-((2-meth lbut-3-yn-2-yl)oxy)propanoate

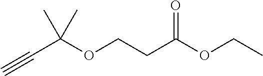

To a stirred and cooled (0° C.) solution of ethyl acrylate (20 g, 200 mmol) and 2-methylbut-3-yn-2-ol (33.6 g, 400 mmol) in tetrahydrofuran (200 mL) was added sodium hydride (6.0 g, 20 mmol) with caution. The mixture was stirred overnight at room temperature, then quenched with water (80 mL) and extracted with ethyl acetate (3×100 mL). The combined organic phases were washed with brine (80 mL), dried over sodium sulfate, and concentrated. The residue was purified by flash chromatography (80 g silica gel column, eluting with 0-60% ethyl acetate in petroleum ether) to give the title compound (16 g, 43%) as colorless oil. ¹H NMR (400 MHz, CDCl₃) δ 4.15 (q, J=7.2 Hz, 2H), 3.83 (t, J=6.8 Hz, 2H), 2.57 (t, J=6.8 Hz, 2H), 2.42 (s, 1H), 1.46 (s, 6H), 1.26 (t, J=7.2 Hz, 3H) ppm.

The following intermediates were prepared utilizing the procedures described for Intermediate 24A.

| Inter. | | | MS m/z |
|---|---|---|---|
| No. | Structure | Name | [M + H]⁺ |
| 24A-1 | 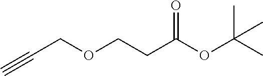 | tert-butyl 3-(prop-2-yn- 1-yloxy)propanoate | 1H NMR (400 MHz, CDCl₃) δ 4.16 (d, J = 2.3 Hz, 2H), 3.76 (t, J = 6.4 Hz, 2H), 2.52 (t, J = 6.4 Hz, 2H), 2.44 (t, J = 2.3 Hz, 1H), 1.46 (s, 9H) ppm. |
| 24A-2 | 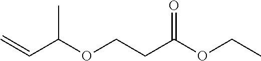 | ethyl 3-(but-3-en-2- yloxy)propanoate | ¹H NMR (400 MHz, CDCl₃) δ 5.97-5.63 (m, 1H), 5.42-5.04 (m, 2H), 4.22-4.07 (m, 2H), 3.91-3.43 (m, 3H), 2.61-2.54 (m, 2H), 1.36-1.11 (m, 6H) ppm. |
| 24A-3 | 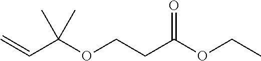 | ethyl 3-((2-methylbut- 3-en-2- yl)oxy)propanoate | ¹HNMR (400 MHz, CDCl₃): 5.86-5.79 (m, 1H), 5.15-5.10 (m, 2H), 4.14 (q, J = 7.2 Hz, 2H), 3.57 (t, J = 6.4 Hz, 2H), 3.57 (t, J = 6.8 Hz, 2H), 1.28-1.24 (m, 9H) ppm. |

Intermediate 24B: 3-((2-Methylbut-3-yn-2-yl)oxy)propan-1-ol

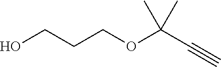

To a stirred and cooled (0° C.) solution of lithium aluminum hydride (2.0 g, 52 mmol) in tetrahydrofuran (120 mL) was added a solution of ethyl 3-(1,1-dimethylprop-2-ynoxy)-propanoate (Intermediate 24A, 8 g, 43.5 mmol) in tetrahydrofuran (50 mL) dropwise. The mixture was stirred at room temperature overnight, then carefully quenched with sodium sulfate decahydrate (6.5 g). The mixture was filtered, and the filtrate was concentrated. The residue was purified by flash chromatography (80 g silica gel column, eluting petroleum ether/ethyl acetate =10/1) to give the title compound (5.68 g, 92%) as colorless oil. ¹H NMR (400 MHz, CDCl₃) δ 3.81-3.73 (m, 2H), 2.53 (brs, 1H), 2.45 (s, 2H), 1.89-1.78 (m, 2H), 1.48 (s, 6H) ppm.

The following intermediate was prepared utilizing the procedures described for Intermediate 24B.

| Inter. | | | |
|---|---|---|---|
| No. | Structure | Name | MS m/z [M + H]⁺ |
| 24B-1 | 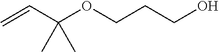 | 3-((2-methylbut-3-en- 2-yl)oxy)propan-1-ol | ¹HNMR (400 MHz, CDCl₃): 5.86-5.78 (m, 1H), 5.16-5.12 (m, 2H), 3.77-3.72 (m, 2H), 3.51 (t, J = 6.0 Hz, 2H), 1.81-1.78 (m, 2H), 1.28 (s, 6H) ppm. |

Intermediate 25: 2-Fluoro-5-((6-fluoro-4-(hydroxymethyl)-1H-indol-5-yl)oxy)benzonitrile

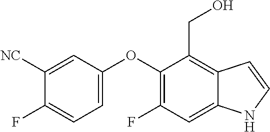 [SMILES: N#Cc1cc(Oc2c(F)cc3[nH]ccc3c2CO)ccc1F]

To a stirred solution of 2-fluoro-5-((6-fluoro-4-formyl-1H-indol-5-yl)oxy)benzonitrile (Intermediate 16-1, 8 g, 26.82 mmol) in ethanol (80 mL) was added sodium borohydride (1.52 g, 40.23 mmol) portion wise. The mixture was stirred at room temperature for 1 hours, quenched with water (100 mL) and extracted with ethyl acetate (3×50 mL). The combined organic extracts were washed with brine, dried over sodium sulfate, and concentrated. The residue was purified by silica gel column (80 g column, eluting with 0-80% ethyl acetate in petroleum ether) to give the title compound (3.8 g, 47%). MS (ESI): 323 m/z [M+Na]⁺, retention time: 1.83 minutes, purity: 98% (254 nm) (LC-MS method 2).

The following intermediate was prepared utilizing the procedures described for Intermediate 25.

| Inter. | | | |
|---|---|---|---|
| No. | Structure | Name | MS m/z [M + H]⁺ |
| 25-1 | 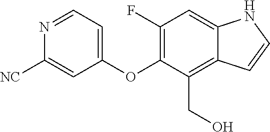 [SMILES: N#Cc1cc(Oc2c(F)cc3[nH]ccc3c2CO)ccn1] | 4-((6-fluoro-4-(hydroxymethyl)- 1H-indol-5-yl)oxy)picolinonitrile | 284 |
| 25-2 | 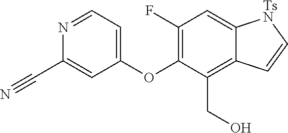 [SMILES: Cc1ccc(S(=O)(=O)n2ccc3c(CO)c(Oc4ccnc(C#N)c4)c(F)cc32)cc1] | 4-((6-fluoro-4-(hydroxymethyl)-1- tosyl-1H-indol-5- yl)oxy)picolinonitrile | 438 |
| 25-3 | 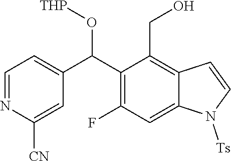 | 4-((6-fluoro-4-(hydroxymethyl)-1- tosyl-1H-indol-5-yl)((tetrahydro- 2H-pyran-2- yl)oxy)methyl)picolinonitrile | 536 m/z [M + H]⁺; RT: 1.24 + 1.26 minutes (LC-MS method 35) |
| 25-4 | 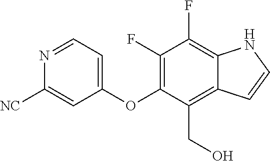 [SMILES: N#Cc1cc(Oc2c(F)c(F)c3[nH]ccc3c2CO)ccn1] | 4-((6,7-difluoro-4- (hydroxymethyl)-1H-indol-5- yl)oxy)picolinonitrile | 302 m/z [M + H]⁺; RT: 1.22 minutes (LC-MS method 2) |

Intermediate 26: 7-Cyano-2-(3-(3-ethoxy-3-oxopropyl)phenyl)-2-methylheptanoic acid

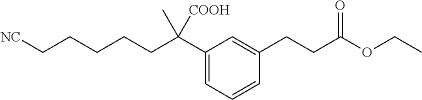 [SMILES: CCOC(=O)CCc1cccc(C(C)(CCCCCC#N)C(=O)O)c1]

To a stirred solution of (E)-7-cyano-2-(3-(3-ethoxy-3-oxoprop-1-en-1-yl)phenyl)-2-methylheptanoic acid (5.5 g, 16.03 mmol) in ethyl acetate (50 mL) was added palladium on carbon (10%, 50% wet, 1 g). The reaction was stirred at room temperature under a hydrogen balloon over the weekend. The mixture was filtered through a pad of Celite. The filtrate was concentrated to give the title compound (4.9 g, 88%) as an oil. MS (ESI): 346 m/z [M+H]⁺, retention time: 1.96 minutes, purity: 92% (254 nm) (LC-MS method 2).

The following intermediates were prepared based on the procedures described for Intermediate 26 and/or Intermediate 26A.

| Inter. | | | MS m/z |
|---|---|---|---|
| No. | Structure | Name | [M + H]⁺ |
| 26-1 | 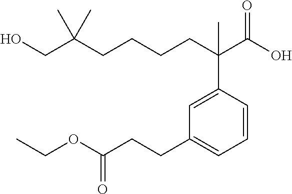 [SMILES: CCOC(=O)CCc1cccc(C(C)(CCCCC(C)(C)CO)C(=O)O)c1] | 2-(3-(3-ethoxy-3-oxopropyl)phenyl)-8-hydroxy- 2,7,7-trimethyloctanoic acid | 379 |
| 26-2 | 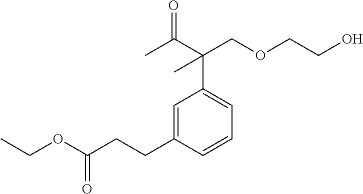 [SMILES: CCOC(=O)CCc1cccc(C(C)(COCCO)C(C)=O)c1] | ethyl 3-(3-(1-(2-hydroxyethoxy)-2-methyl-3- oxobutan-2-yl)phenyl)propanoate | 323 |
| 26-3 | 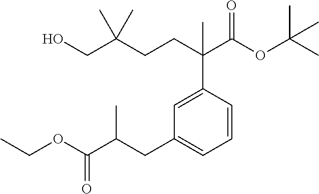 [SMILES: CCOC(=O)C(C)Cc1cccc(C(C)(CCC(C)(C)CO)C(=O)OC(C)(C)C)c1] | tert-butyl 2-(3-(3-ethoxy-2-methyl-3- oxopropyl)phenyl)-6-hydroxy-2,5,5- trimethylhexanoate | 443 [M + Na]⁺ |

Intermediate 26A: (E)-7-Cyano-2-(3-(3-ethoxy-3-oxoprop-1-en-1-yl)phenyl)-2-methylheptanoic acid

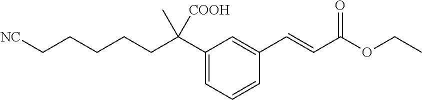 [SMILES: CCOC(=O)/C=C/c1cccc(C(C)(CCCCCC#N)C(=O)O)c1]

To a stirred and degassed solution of 7-cyano-2-(3-iodophenyl)-2-methylheptanoic acid (Intermediate 7-9, 5 g, 13.47 mmol) in dimethylformamide (50 mL) was added triethylamine (9.32 mL, 67.35 mmol), ethyl acrylate (2.69 g, 26.95 mmol), tri(o-tolyl)phosphine (819 mg, 2.694 mmol), palladium(II) acetate (0.302 g, 1.34 mmol). The reaction was stirred at 110° C. overnight, then cooled to room temperature and diluted with ethyl acetate (200 mL). The solution was washed with 1 N hydrochloric acid, brine, dried with sodium sulfate, and concentrated. The residue was purified by silica gel column chromatography (eluting with 0-30% ethyl acetate in petroleum ether) to give the title compound (4.3 g, 93%) as a yellow oil. MS (ESI): 344 m/z [M+H]⁺, retention time: 1.97 minutes, purity: 98% (254 nm) (LC-MS method 2).

The following intermediate was prepared based on the procedures described for Intermediate 26A.

| Inter. | | | MS m/z |
|---|---|---|---|
| No. | Structure | Name | [M + H]⁺ |
| 26A-1 | 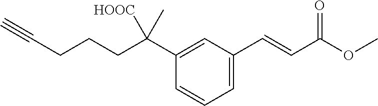 [SMILES: C#CCCCC(C)(C(=O)O)c1cccc(/C=C/C(=O)OC)c1] | (E)-2-(3-(3-methoxy-3- oxoprop-1-en-1- yl)phenyl)-2- methylhept-6-ynoic acid | 301 |

Intermediate 27: Tert-Butyl((6-iodo-2,2-dimethylhexyl)oxy)dimethylsilane

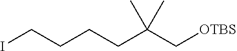 [SMILES: CC(C)(CCCCI)CO[Si](C)(C)C(C)(C)C]

To a stirred solution of tert-butyl((6-iodo-2,2-dimethylhexyl)oxy)dimethylsilane (Intermediate 29B, 27.00 g, 96.8 mmol) in acetone (300 mL) was added sodium iodide (43.50 g, 290 mmol) at room temperature. The mixture was stirred at 60° C. for 16 hours. The solvent was evaporated. The residue was dissolved in ethyl acetate (500 mL), washed with water (2×300 mL), dried over anhydrous sodium sulfate and concentrated. The crude residue was purified by flash chromatography (330 g silica gel column, petroleum ether) to give the title compound as a colorless oil (32.00 g, 89.3%). ¹H NMR (400 MHz, CDCl₃) 3.25-3.13 (m, 4H), 1.80-1.68 (m, 2H), 1.39-1.27 (m, 2H), 1.23-1.16 (m, 2H), 0.87 (s, 9H), 0.80 (s, 6H), −0.01 (s, 6H) ppm.

Intermediate 27A: Methyl 6-chloro-2,2-dimethyl-hexanoate

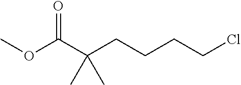

To a stirred and cooled (−78° C.) solution of methyl 2-methylpropanoate (10.30 g, 101 mmol) in tetrahydrofuran (200 mL) was added lithium diisopropylamide (50.4 mL, 101 mmol) dropwise. After stirring at −78° C. for 1 hour, 1-chloro-4-iodo-butane (20.00 g, 91.5 mmol) was added. The mixture was stirred at −78° C. for 1 hour and then allowed to warm to room temperature and stirred for an additional 16 hours. The reaction was quenched with 1M hydrochloric acid (200 mL) and extracted with ethyl acetate (2×200 mL). The combined organic phases were washed with water, brine, dried over sodium sulfate and concentrated. The crude residue was purified by flash silica gel chromatography (50% ethyl acetate in petroleum ether) to give the title compound as a light-yellow oil (16.00 g, 83%). MS (ESI): 193 m/z [M+H]⁺.

The following intermediate was prepared based on the procedures described for Intermediate 27A.

| Inter. | | | |
|---|---|---|---|
| No. | Structure | Name | MS m/z [M + H]⁺ |
| 27A-1 | 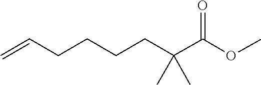 | methyl 2,2- dimethyloct-7- enoate | ¹H NMR (400 MHz, CDCl₃): δ 5.84-5.74 (m, 1H), 5.00 (dd, J = 17.2, 2.0 Hz, 1H), 4.93 (dd, J = 10.4, 1.2 Hz, 1H), 3.65 (s, 3H), 2.07-1.99 (m, 2H), 1.55-1.47 (m, 2H), 1.38-1.34 (m, 2H), 1.28-1.19 (m, 2H), 1.16 (s, 6H) ppm. |

Intermediate 27B: 6-chloro-2,2-dimethylhexan-1-ol

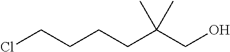

To a stirred and cooled (−78° C.) solution of methyl 6-chloro-2,2-dimethyl-hexanoate (Intermediate 27A, 24.00 g, 125 mmol) in tetrahydrofuran (200 mL) was added lithium aluminum hydride (7.09 g, 187 mmol) dropwise over 30 minutes. The mixture was stirred for 3 hours at −78° C., then quenched with 7 mL of water, warmed to 0° C., followed by 7 mL of 15% sodium hydroxide, and finally 21 mL of water. The mixture was stirred at room temperature for 15 minutes and then filtered. The filtrate was concentrated to give the title compound as an oil (18.00 g, 88%). ¹H NMR (400 MHz, CDCl₃) δ 3.55 (t, J=6.7 Hz, 2H), 3.53 (s, 2H), 1.80-1.73 (m, 2H), 1.4-1.39 (m, 2H), 1.28-1.26 (m, 2H), 0.86 (s, 6H) ppm.

The following intermediate was prepared based on the procedures described for Intermediate 27B.

| Inter. | | | |
|---|---|---|---|
| No. | Structure | Name | MS m/z [M + H]⁺ |
| 27B-1 | 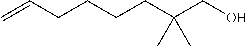 | 2,2-dimethyloct- 7-en-1-ol | ¹H NMR (400 MHz, CDCl₃) δ 5.84- 5.77 (m, 1H), 5.00 (dd, J = 17.2, 1.2 Hz, 1H), 4.94 (d, J = 10.0 Hz, 1H), 3.31 (s, 2H), 2.09-2.03 (m, 2H), |
| | | | 1.39-1.16 (m, 6H), 0.86 (s, 6H) ppm |

Intermediate 27C: Tert-Butyl-(6-chloro-2,2-dimethyl-hexoxy)-dimethylsilane

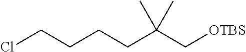 [SMILES: CC(C)(CCCCCl)CO[Si](C)(C)C(C)(C)C]

To a stirred solution of 6-chloro-2,2-dimethyl-hexan-1-ol (Intermediate 27B, 18.00 g, 109 mmol) in dichloromethane (300 mL) was added tert-butyldimethylsilyl chloride (19.80 g, 131 mmol) and imidazole (14.9 g, 219 mmol). The mixture was stirred at room temperature for 2 hours, then diluted with dichloromethane (200 mL). The solution was washed with water (2×100 mL), brine, dried over anhydrous sodium sulfate and concentrated. The crude residue was purified by flash chromatography (120 g silica gel column, petroleum ether) to give the title compound as an oil (27.00 g, 89%). ¹H NMR (400 MHz, CDCl₃) δ 3.52 (t, J=6.7 Hz, 2H), 3.21 (s, 2H), 1.76-1.68 (m, 2H), 1.35 (dt, J=10.7, 7.9 Hz, 2H), 1.23-1.18 (m, 2H), 0.87 (s, 9H), 0.80 (s, 6H), −0.01 (s, 6H) ppm.

The following intermediate was prepared based on the procedures described for Intermediate 27C.

| Inter. | | | |
|---|---|---|---|
| No. | Structure | Name | MS m/z [M + H]⁺ |
| 27C-1 | 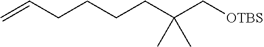 [SMILES: C=CCCCCC(C)(C)CO[Si](C)(C)C(C)(C)C] | tert-butyl((2,2- dimethyloct-7-en-1- yl)oxy)dimethylsilane | ¹H NMR (400 MHz, CDCl₃) δ 5.83-5.75 (m, 1H), 4.98 (dd, J = 17.2, 1.2 Hz, 1H), 4.92 (d, J = 10.4 Hz, 1H), 3.20 (s, 2H), 2.03 (q, J = 7.2 Hz, 2H), 1.55-1.16 (m, |
| | | | 6H), 0.90 (s, 9H), 0.84 (s, 6H), 0.03 (s, |
| | | | 6H) ppm |

Intermediate 28: Methyl 2-(3-iodophenyl)-2,7,7-trimethylnon-8-ynoate

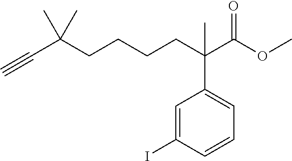 [SMILES: C#CC(C)(C)CCCCC(C)(C(=O)OC)c1cccc(I)c1]

To a stirred solution of methyl 2-(3-iodophenyl)-2,7,7-trimethyl-8-oxooctanoate (Intermediate 28B, 5 g, 12 mmol) in methanol (40 mL) was added potassium carbonate (4.98 g, 36 mmol), and 1-diazo-1-dimethoxyphosphoryl-propan-2-one (3.23 g, 16.8 mmol). The mixture was stirred at room temperature for 2 hours under nitrogen, quenched with water (120 mL) and extracted with dichloromethane (3×50 mL). The combined organic extracts were dried over sodium sulfate and concentrated. The residue was purified by silica gel column chromatography (eluting with petroleum ether/ethyl acetate=10/1) to give the title compound (3.71 g, 75%) as an oil. MS (ESI): 435 m/z [M+Na]⁺, retention time: 2.40 minutes, purity: 97% (214 nm) (LC-MS method 2).

The following intermediates were prepared based on the procedures described for Intermediate 28.

| Inter. | | | |
|---|---|---|---|
| No. | Structure | Name | MS m/z [M + H]⁺ |
| 28-1 | 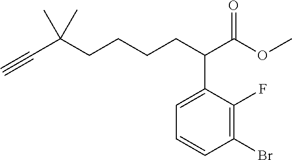 [SMILES: C#CC(C)(C)CCCCC(C(=O)OC)c1cccc(Br)c1F] | methyl 2-(3-bromo-2- fluorophenyl)-7,7- dimethylnon-8-ynoate | MS: 391, 393 m/z [M + Na]⁺; ¹H NMR (400 MHz, CDCl₃) δ 7.47-7.44 (m, 1H), 7.30-7.27 (m, 1H), 7.01 (td, J = 7.9, 0.9 Hz, 1H), 3.94 (t, J = 7.6 Hz, 1H), 3.68 (s, 3H), 2.11-2.08 (m, 2H), 1.86-1.71 (m, 1H), 1.49-1.46 (m, 2H), 1.38-1.23 (m, 4H), 1.18 (s, 6H) ppm. |
| 28-2 | 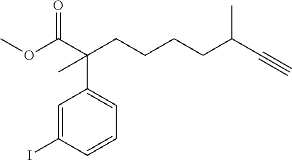 [SMILES: C#CC(C)CCCCC(C)(C(=O)OC)c1cccc(I)c1] | methyl 2-(3- iodophenyl)-2,7- dimethylnon-8-ynoate | 421[M + Na]⁺ |
| 28-3 | 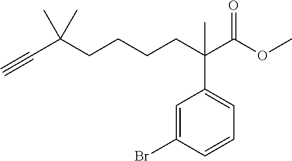 [SMILES: C#CC(C)(C)CCCCC(C)(C(=O)OC)c1cccc(Br)c1] | methyl 2-(3- bromophenyl)-2,7,7- trimethylnon-8- ynoate | 387, 389[M + Na]⁺ |

Intermediate 28A: Methyl 8-hydroxy-2-(3-iodophenyl)-2,7,7-trimethyloctanoate

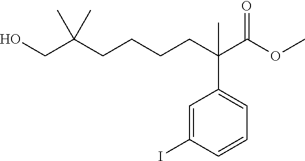 [SMILES: COC(=O)C(C)(CCCCC(C)(C)CO)c1cccc(I)c1]

To a stirred solutions of methyl 8-[tert-butyl(dimethyl)silyl]oxy-2-(3-iodophenyl)-2,7,7-trimethyl-octanoate (Intermediate 7A-9, 10.8 g, 20.3 mmol) in tetrahydrofuran (15 mL) was added tetra butyl ammonium fluoride (1M in tetrahydrofuran, 61 mL, 61 mmol). The mixture was stirred at room temperature for 16 hours and concentrated. The residue was treated with brine (30 mL), acidified to pH ˜7 with 2 M hydrochloric acid, then extracted with ethyl acetate (3×80 mL). The combined organic phases were dried over anhydrous sodium sulfate and concentrated. The residue was purified by silica gel column chromatography (eluting with petroleum ether/ethyl acetate=4/1) to give the title compound (6.2 g, 70%) as an oil. MS (ESI): 441 m/z [M+Na]⁺, retention time: 2.22 minutes, purity: 87% (214 nm) (LC-MS method 2).

Intermediate 28B: Methyl 2-(3-iodophenyl)-2,7,7-trimethyl-8-oxooctanoate

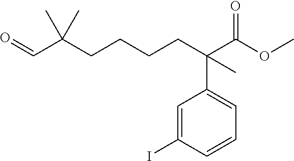 [SMILES: COC(=O)C(C)(CCCCC(C)(C)C=O)c1cccc(I)c1]

To a stirred and cooled (0° C.) solution of methyl 8-hydroxy-2-(3-iodophenyl)-2,7,7-trimethyl-octanoate (Intermediate 28A, 6 g, 14.3 mmol) in dichloromethane (40 mL) was added pyridinium chlorochromate (6.18 g, 28.7 mmol) and silica (6.18 g). The reaction was stirred at room temperature for two hours, quenched with water (40 mL) and extracted with dichloromethane (3×60 mL). The combined organic extracts were dried over sodium sulfate and concentrated. The residue was purified by silica gel column chromatography (eluting with petroleum ether/ethyl acetate=4/1) to give the title compound (5 g, 83%) as an oil. MS (ESI): 417 m/z [M+H]⁺.

The following intermediate was prepared based on the procedures described for Intermediate 28B.

| Inter. | | | MS m/z |
|---|---|---|---|
| No. | Structure | Name | [M + H]⁺ |
| 28B-1 | 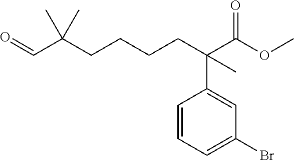 [SMILES: COC(=O)C(C)(CCCCC(C)(C)C=O)c1cccc(Br)c1] | methyl 2-(3-bromophenyl)- 2,7,7-trimethyl-8- oxooctanoate | 369, 371 |

Intermediate 29: Ethyl 2-(5-(3-carbamimidoyl-4-fluorophenoxy)-6-fluoro-1H-indol-4-yl)acetate

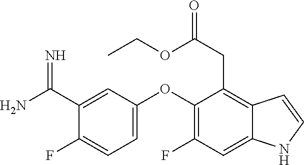 [SMILES: CCOC(=O)Cc1c(Oc2ccc(F)c(C(=N)N)c2)c(F)cc2[nH]ccc12]

To a stirred solution of ethyl 2-(5-(3-(N-acetoxycarbamimidoyl)-4-fluorophenoxy)-6-fluoro-1H-indol-4-yl)acetate (Intermediate 29C, 2.066 g, 4.8 mmol) in acetic acid (11 mL) was added palladium on carbon (10%, 50% wet, 214 mg). The reaction was stirred at room temperature overnight under hydrogen balloon. The pH was adjusted to ˜8 with saturated sodium bicarbonate. The mixture was diluted with ethyl acetate (300 mL), washed with water, brine, dried over sodium sulfate, filtered, and concentrated. The residue was purified by automated silica gel column chromatography (40 g column, eluting with 0-10% methanol in dichloromethane) to give the title compound (1.37 g, 76%) as a green-yellow solid. MS (ESI): 374 m/z [M+H]⁺, retention time: 1.57 minutes, purity: 78% (254 nm) (LC-MS method 2).

The following intermediate was prepared based on the procedures described for Intermediate 29.

| Inter. | | | |
|---|---|---|---|
| No. | Structure | Name | MS m/z [M + H]⁺ |
| 29-1 | 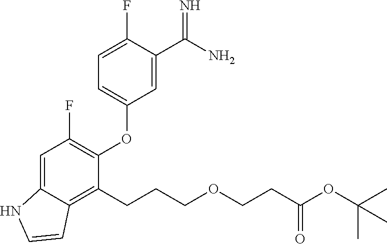 [SMILES: CC(C)(C)OC(=O)CCOCCCc1c(Oc2ccc(F)c(C(=N)N)c2)c(F)cc2[nH]ccc12] | tert-butyl 3-(3-(5-(3- carbamimidoyl-4- fluorophenoxy)-6- fluoro-1H-indol-4- yl)propoxy)propanoate | MS: 474 m/z [M + H]⁺. ¹H NMR (400 MHz, CD₃OD) δ 7.30 (d, J = 3.2 Hz, 1H), 7.24- 7.17 (m, 3H), 7.03-6.99 (m, 1H), 6.58 (d, J = 3.2 Hz, 1H), 3.62 (t, J = 6.1 Hz, 2H), 3.44 (t, J = 6.2 Hz, 2H), 2.96-2.83 (m, 2H), 2.45 (t, J = 6.1 Hz, 2H), 1.92-1.81 (m, 2H), 1.45 (s, 9H) ppm. |

Intermediate 29A: Ethyl 2-(5-(3-cyano-4-fluorophenoxy)-6-fluoro-1-tosyl-1H-indol-4-yl)acetate

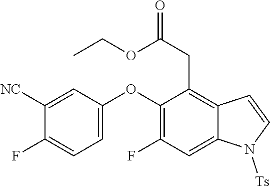 [SMILES: CCOC(=O)Cc1c(Oc2ccc(F)c(C#N)c2)c(F)cc2c1ccn2S(=O)(=O)c1ccc(C)cc1]

In a glovebox, to a stirred solution of 5-((4-bromo-6-fluoro-1-tosyl-1H-indol-5-yl)oxy)-2-fluorobenzonitrile (Intermediate 4, 8 g, 16 mmol) in N,N-dimethylformamide (65 mL) was added ethyl 2-tributylstannylacetate (15.1 g, 40 mmol), dichlorobis(tri-o-tolylphosphine)palladium(II) (1.261 g, 1.6 mmol) and zinc bromide (7.2 g, 32 mmol). The reaction was stirred at 100° C. under nitrogen overnight, cooled to room temperature and diluted with ethyl acetate (800 mL). The mixture was washed with water, brine, dried over sodium sulfate, and concentrated. The residue was purified by automated silica gel column chromatography (80 g column, eluting with 0-50% ethyl acetate in petroleum ether) to give the title compound (4.6 g, 57%) as a white solid. MS (ESI): 533 m/z [M+Na]⁺, retention time: 2.20 minutes, purity: 95% (214 nm) (LC-MS method 2).

Intermediate 29B: Ethyl 2-(5-(3-cyano-4-fluorophenoxy)-6-fluoro-1H-indol-4-yl)acetate

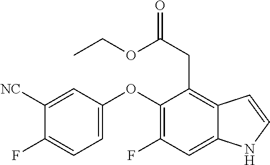 [SMILES: CCOC(=O)Cc1c(Oc2ccc(F)c(C#N)c2)c(F)cc2[nH]ccc12]

To a stirred solution of ethyl 2-(5-(3-cyano-4-fluorophenoxy)-6-fluoro-1-tosyl-1H-indol-4-yl)acetate (Intermediate 29A, 1.93 g, 3.8 mmol) in tetrahydrofuran (15.9 mL) was added tetrabutyl-ammonium fluoride (22.7 mL, 22.7 mmol). The reaction was stirred at 75° C. overnight, cooled to room temperature, and diluted with ethyl acetate (500 mL). The mixture was washed with water, brine, dried over sodium sulfate, and concentrated. The residue was purified by automated silica gel column chromatography (40 g silica gel column, eluting with 0-100% ethyl acetate in petroleum ether) to give the title compound (1.027 g, 76%) as a yellow solid. MS (ESI): 357 m/z [M+H]⁺, retention time: 2.09 minutes, purity: 87% (214 nm) (LC-MS method 5).

Intermediate 29C: Ethyl 2-(5-(3-(N-acetoxycarbamimidoyl)-4-fluorophenoxy)-6-fluoro-1H-indol-4-yl)acetate

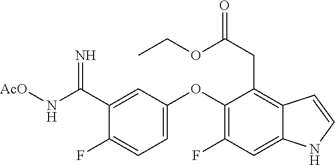 [SMILES: CCOC(=O)Cc1c(Oc2ccc(F)c(C(=N)NOC(C)=O)c2)c(F)cc2[nH]ccc12]

To a stirred solution of ethyl 2-(5-(3-cyano-4-fluorophenoxy)-6-fluoro-1H-indol-4-yl)acetate (Intermediate 29B, 2.063 g, 5.8 mmol) in dry methanol (22 mL) was added hydroxylamine hydrochloride (1.2 g, 17.4 mmol) and triethylamine (2.4 mL, 17.4 mmol). The reaction was stirred at room temperature overnight, then diluted with ethyl acetate (500 mL). The mixture was washed with water, brine, dried over sodium sulfate, and concentrated. The crude product, ethyl 2-(6-fluoro-5-(4-fluoro-3-(N-hydroxycarbamimidoyl)phenoxy)-1H-indol-4-yl)acetate, was used for the next step without further purification. MS (ESI): 390 m/z [M+H]⁺, retention time: 1.63 minutes, purity: 92% (254 nm) (LC-MS method 5).

To a stirred and cooled (0° C.) solution of the above crude product (2.159 g, 5.55 mmol) in acetic acid (21 mL) was added acetic anhydride (0.79 mL, 8.33 mmol). The reaction was stirred at room temperature overnight, then diluted with ethyl acetate (300 mL). The mixture was washed with brine, dried over sodium sulfate, and concentrated. The residue was purified by silica gel column chromatography (40 g column, eluting with 0-100% ethyl acetate in petroleum ether) to give the title compound (2.066 g, 86% two steps) as a yellow solid. MS (ESI): 432 m/z [M+H]⁺, retention time: 1.83 minutes, purity: 97% (254 nm) (LC-MS method 2).

Intermediate 30: 5-((4-((4-Bromothiazol-2-yl)methyl)-6-fluoro-1H-indol-5-yl)oxy)-2-fluorobenzimidamide

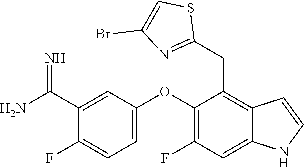 [SMILES: N=C(N)c1cc(Oc2c(F)cc3[nH]ccc3c2Cc2nc(Br)cs2)ccc1F]

To ethyl 5-((4-((4-bromothiazol-2-yl)methyl)-6-fluoro-1H-indol-5-yl)oxy)-2-fluorobenzimidate (Intermediate 30C, 630 mg, 1.28 mmol) in a sealed tube was added ammonia in ethanol (7 M, 18 mL). The resulting mixture was then stirred at 60° C. overnight and concentrated. The residue was partitioned between water (50 mL) and ethyl acetate (50 mL). The separated organic layer, combined with two additional ethyl acetate extracts, was washed with brine, dried over sodium sulfate, and concentrated to give the title compound (500 mg, 84%), which was used for the next step without further purification. MS (ESI): 463, 465 m/z [M+H]⁺, retention time: 1.68 minutes, purity: 85% (214 nm) (LC-MS method 2).

The following intermediate was prepared based on the procedures described for Intermediate 30 and/or for Intermediate 30A to 30C.

| Inter. | | | MS m/z |
|---|---|---|---|
| No. | Structure | Name | [M + H]⁺ |
| 30-1 | 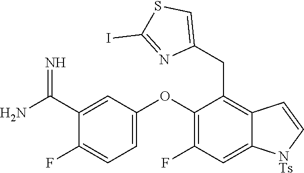 [SMILES: Cc1ccc(S(=O)(=O)n2ccc3c(Cc4csc(I)n4)c(Oc4ccc(F)c(C(=N)N)c4)c(F)cc32)cc1] | 2-fluoro-5-((6-fluoro-4-((2- iodothiazol-4-yl)methyl)-1- tosyl-1H-indol-5-yl)oxy) benzimidamide | 665 |

Intermediate 30A: 5-((4-((4-Bromothiazol-2-yl)methyl)-6-fluoro-1-(phenylsulfonyl)-1H-indol-5-yl)oxy)-2-fluorobenzonitrile

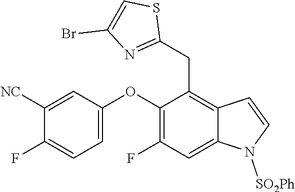

To a stirred and cooled (−78° C.) solution of 2,4-dibromothiazole (1.25 g, 5.2 mmol) in tetrahydrofuran (30 mL) was added n-butyllithium (2.5 M in hexanes, 2.07 mL, 5.2 mmol). The reaction was stirred at −78° C. for 0.5 hours. This solution was added drop wise to a stirred solution of 5-[1-(benzenesulfonyl)-4-(bromomethyl)-6-fluoro-indol-5-yl]oxy-2-fluoro-benzonitrile (Intermediate 8, 2.6 g, 5.2 mmol) in tetrahydrofuran (20 mL) at −78° C. The mixture was stirred at this temperature for another 0.5 hours, then quenched with saturated ammonium chloride (50 mL). The solution was warmed to room temperature, extracted with ethyl acetate (3×50 mL). The combined organic phases were washed with brine (50 mL), dried over sodium sulfate, and concentrated. The residue was purified by column chromatography on silica gel, eluting with dichloromethane/methanol (98:2) to afford the title compound (0.4 g, 13%) as an oil and 1 g of the starting material was recovery. MS (ESI): No mass peak shown, retention time: 2.19 minutes, purity: 68% (214 nm) (LC-MS method 2).

Intermediate 30B: 5-((4-((4-Bromothiazol-2-yl)methyl)-6-fluoro-1H-indol-5-yl)oxy)-2-fluorobenzonitrile

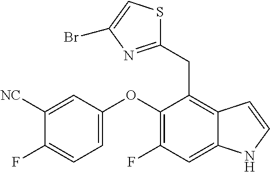 [SMILES: N#Cc1cc(Oc2c(F)cc3[nH]ccc3c2Cc2nc(Br)cs2)ccc1F]

To a solution of 5-((4-((4-bromothiazol-2-yl)methyl)-6-fluoro-1-(phenylsulfonyl)-1H-indol-5-yl)oxy)-2-fluorobenzonitrile (Intermediate 30A, 1.1 g, 1.9 mmol) in methanol (20 mL) was added potassium carbonate (778 mg, 5.6 mmol), the resulting mixture stirred for 3 hours at 60° C., quenched with water (50 mL), and extracted with ethyl acetate (3×50 mL). The combined organic phases were washed with water, brine, dried over sodium sulfate, and concentrated. The residue was purified by column chromatography on silica gel, eluting with ethyl acetate/petroleum ether (40:60) to afford the title compound (700 mg, 84%) as an oil. MS (ESI): 446,448 m/z [M+H]⁺, retention time: 2.05 minutes, purity: >99% (254 nm) (LC-MS method 2).

Intermediate 30C: Ethyl 5-((4-((4-bromothiazol-2-yl)methyl)-6-fluoro-1H-indol-5-yl)oxy)-2-fluorobenzimidate

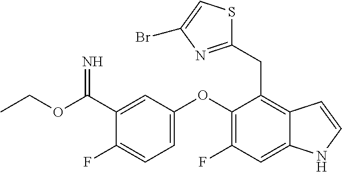 [SMILES: CCOC(=N)c1cc(Oc2c(F)cc3[nH]ccc3c2Cc2nc(Br)cs2)ccc1F]

A mixture of 5-((4-((4-bromothiazol-2-yl)methyl)-6-fluoro-1H-indol-5-yl)oxy)-2-fluoro-benzonitrile (600 mg, 1.34 mmol) and hydrogen chloride in ethanol (33%, 10 mL) in a sealed tube was stirred at room temperature overnight and concentrated to give the crude title compound (630 mg, 100%), which was used for the next step without further purification. MS (ESI): 492, 494 m/z [M+H]⁺, retention time: 1.75 minutes, purity: 80% (254 nm) (LC-MS method 2).

Intermediate 31: 7-Bromo-2-(3-iodophenyl)-2,5,5-trimethylheptanoic acid

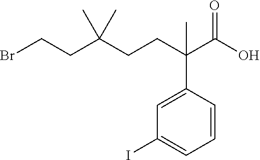 [SMILES: CC(C)(CCBr)CCC(C)(C(=O)O)c1cccc(I)c1]

To a solution of methyl 7-bromo-2-(3-iodophenyl)-2,5,5-trimethylheptanoate (Intermediate 30A, 9.0 g, 19.3 mmol) in 90 mL of methanol was added sodium hydroxide (3.8 g, 96.6 mmol) in water (20 ml). The reaction was stirred at 60° C. for 2 hours and concentrated. The residue was re-dissolved into water (100 mL) and acidified with hydrochloric acid (conc.) to pH ˜3. The mixture was extracted with ethyl acetate (2×200 mL). The combined organic layers were washed with brine, dried over sodium sulfate, and concentrated. The crude product was purified by silica gel flash column (eluting with petroleum ether/ethyl acetate=1/1) to give the title compound (8.0 g, 87%) as a solid. MS (ESI): 453, 455 m/z [M+H]⁺.

Intermediate 31A: Methyl 7-bromo-2-(3-iodophenyl)-2,5,5-trimethylheptanoate

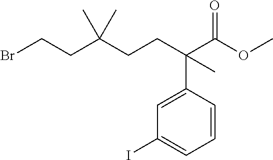 [SMILES: COC(=O)C(C)(CCC(C)(C)CCBr)c1cccc(I)c1]

To a solution of methyl 2-(3-iodophenyl)-2,5,5-trimethyl-7-(tosyloxy)heptanoate (Intermediate 7A-12, 15.0 g, 26.9 mmol) in 200 mL of acetone was added lithium bromide (5.70 g, 67 mmol) under ice bath conditions. The mixture was stirred at reflux temperature overnight and concentrated. The residue was purified by chromatographed over 10% ethyl acetate in petroleum ether to give the title compound (10.0 g, 80%) as a colorless oil. ¹H NMR (400 MHz, CDCl₃) δ 7.62 (t, J=1.7 Hz, 1H), 7.60-7.54 (m, 1H), 7.29-2.23 (m, 1H), 7.06 (t, J=7.9 Hz, 1H), 3.66 (s, 3H), 3.36-3.21 (m, 2H), 1.90-1.80 (m, 1H), 1.87-1.78 (m, 3H), 1.50 (s, 3H), 1.00-0.96 (m, 2H), 0.88-0.84 (m, 6H) ppm.

Intermediate 32: 5-(Chloromethoxy)-4,4-dimethylpent-1-yne

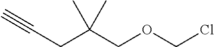

To a stirred and cooled (0° C.) solution of 2,2-dimethylpent-4-yn-1-ol (8.9 g, 79.46 mmol) in dichloromethane (20 mL) was added paraformaldehyde (3.58 g, 119.20 mmol). The mixture was stirred at 0° C. for 3 hours, with hydrogen chloride gas bubbled through the mixture persistently. The mixture was filtered to remove any solid. The filtrate was dried over calcium chloride and concentrated to give the crude title compound (11 g, 87%) as a white liquid. The crude compound was used for the next step without further purification. ¹H NMR (400 MHz, CDCl₃) δ 5.53-5.49 (m, 2H), 3.51 (s, 2H), 3.35 (s, 2H), 1.06-1.04 (m, 6H) ppm.

Intermediate 33: 1-Bromo-4-((2,2-dimethylpent-4-yn-1-yl)oxy)-3-(3-iodophenyl)-3-methylbutan-2-one

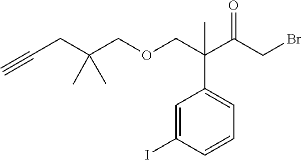 [SMILES: C#CCC(C)(C)COCC(C)(C(=O)CBr)c1cccc(I)c1]

To a stirred solution of 4-((2,2-dimethylpent-4-yn-1-yl)oxy)-3-(3-iodophenyl)-3-methylbutan-2-one (Intermediate 33B, 2.7 g, 6.78 mmol) in tetrahydrofuran (30 mL) was added pyridine hydrobromide perbromide (2.4 g, 7.46 mmol). The reaction was stirred at room temperature for 2 hours, then quenched with 60 mL of water, and extracted with ethyl acetate (3×60 mL). The organic layer was dried over sodium sulfate and concentrated. The residue was purified by silica gel column chromatography (eluting with petroleum ether/ethyl acetate=1/1) to give the title compound (2.2 g, 69%) as a light-yellow liquid. MS (ESI): 477, 479 m/z [M+H]⁺, retention time: 2.06 minutes, purity: 92% (214 nm) (LC-MS method 7).

The following intermediates were prepared based on the procedures described for Intermediate 33.

| Inter. | | | MS m/z |
|---|---|---|---|
| No. | Structure | Name | [M + H]⁺ |
| 33-1 | 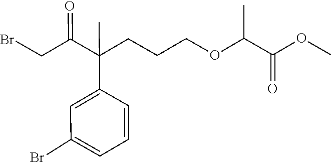 [SMILES: COC(=O)C(C)OCCCC(C)(C(=O)CBr)c1cccc(Br)c1] | methyl 2-((6-bromo-4-(3- bromophenyl)-4-methyl-5- oxohexyl)oxy)propanoate | 473 [M + Na]⁺ |
| 33-2 | 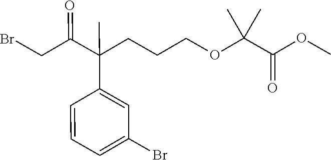 [SMILES: COC(=O)C(C)(C)OCCCC(C)(C(=O)CBr)c1cccc(Br)c1] | methyl 2-((6-bromo-4-(3- bromophenyl)-4-methyl-5- oxohexyl)oxy)-2-methylpropanoate | 465 |
| 33-3 | 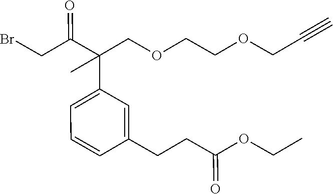 [SMILES: C#CCOCCOCC(C)(C(=O)CBr)c1cccc(CCC(=O)OCC)c1] | ethyl 3-(3-(4-bromo-2-methyl- 3-oxo-1-(2-(prop-2-yn-1- yloxy)ethoxy)butan-2-yl) phenyl)propanoate | 439, 441 |
| 33-4 | 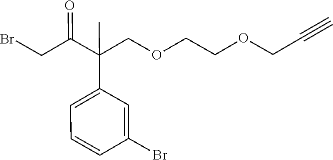 [SMILES: C#CCOCCOCC(C)(C(=O)CBr)c1cccc(Br)c1] | 1-bromo-3-(3-bromophenyl)- 3-methyl-4-(2-(prop-2-yn-1- yloxy)ethoxy)butan-2-one | 419 |

Intermediate 33A: 3-((2,2-Dimethylpent-4-yn-1-yl)oxy)-2-(3-iodophenyl)-N-methoxy-N,2-dimethylpropanamide

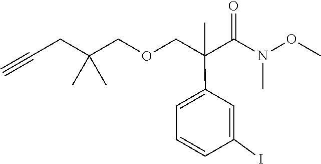 [SMILES: C#CCC(C)(C)COCC(C)(C(=O)N(C)OC)c1cccc(I)c1]

To a stirred solution of 3-((2,2-dimethylpent-4-yn-1-yl)oxy)-2-(3-iodophenyl)-2-methylpropanoic acid (Intermediate 7-15, 5 g, 12.5 mmol) in dimethylformamide (30 mL) was added N,O-dimethylhydroxylamine (1.83 g, 18.75 mmol), 1-[bis(dimethylamino) methylene]-1H-1,2,3-triazolo[4,5-b]pyridinium 3-oxide hexafluorophosphate (HATU, 5.7 g, 15 mmol) and triethylamine (3.8 g, 37.5 mmol). The mixture was stirred at room temperature for 2 hours, quenched with 80 mL of water, and extracted with ethyl acetate (3×100 mL). The combined organic layers were dried over sodium sulfate and concentrated. The residue was purified by silica gel column chromatography (eluting with petroleum ether/ethyl acetate=4/1) to give the title compound (3.9 g, 71%) as a light-yellow liquid. MS (ESI): 444 m/z [M+H]⁺, retention time: 1.99 minutes, purity: 98% (214 nm) (LC-MS method 7).

The following intermediates were prepared based on the procedures described for Intermediate 33A.

| Inter. | | | |
|---|---|---|---|
| No. | Structure | Name | MS m/z [M + H]⁺ |
| 33A-1 | 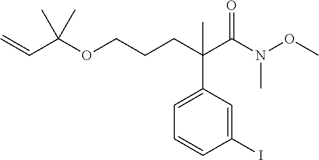 [SMILES: C=CC(C)(C)OCCCC(C)(C(=O)N(C)OC)c1cccc(I)c1] | 2-(3-iodophenyl)- N-methoxy-N,2- dimethyl-5-((2- methylbut-3-en-2- yl)oxy)pentanamide | 468 [M + Na]⁺ |
| 33A-2 | 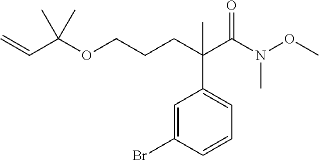 [SMILES: C=CC(C)(C)OCCCC(C)(C(=O)N(C)OC)c1cccc(Br)c1] | 2-(3-bromophenyl)-N- methoxy-N,2-dimethyl- 5-((2-methylbut-3- en-2-yl)oxy) pentanamide | 420, 422 [M + Na]⁺ |
| 33A-3 | 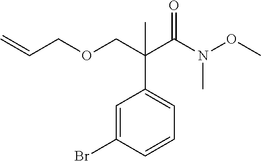 [SMILES: C=CCOCC(C)(C(=O)N(C)OC)c1cccc(Br)c1] | 3-(allyloxy)-2-(3- bromophenyl)-N- methoxy-N,2- dimethylpropanamide | 342, 344 |
| 33A-4 | 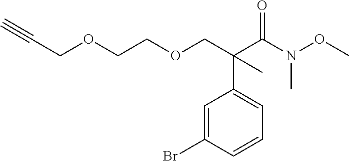 [SMILES: C#CCOCCOCC(C)(C(=O)N(C)OC)c1cccc(Br)c1] | 2-(3-bromophenyl)-N- methoxy-N,2-dimethyl- 3-(2-(prop-2-yn-1- yloxy)ethoxy) propanamide | MS: 384, 386 m/z [M + H]⁺; ¹H NMR (400 MHz, CDCl₃) δ 7.42 (s, 1H), 7.39-7.32 (m, 1H), 7.22-7.19 (m, 1H), 7.19 (s, 1H), 4.09 (d, J = 2.0 Hz, 2H), 3.92-3.85 (m, 2H), 3.64- 3.48 (m, 4H), 3.11 (s, 3H), 2.80 (s, 3H), 2.40 (t, J = 2.4 Hz, 1H), 1.65 (s, 3H) ppm. |

Intermediate 33B: 4-((2,2-Dimethylpent-4-yn-1-yl)oxy)-3-(3-iodophenyl)-3-methylbutan-2-one

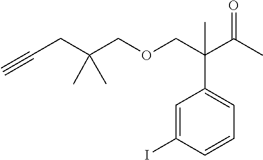 [SMILES: C#CCC(C)(C)COCC(C)(C(C)=O)c1cccc(I)c1]

To a stirred solution of 3-((2,2-dimethylpent-4-yn-1-yl)oxy)-2-(3-iodophenyl)-N-methoxy-N,2-dimethylpropanamide (3.9 g, 8.80 mmol) in tetrahydrofuran (50 mL) was added methyl magnesium bromide (3 M in ether, 44.02 mmol, 15 mL). The reaction was stirred at room temperature overnight, cooled to 0° C., and quenched with 100 mL of water. The mixture was extracted with ethyl acetate (3×120 mL). The combined organic layers were dried over sodium sulfate and concentrated. The residue was purified by silica gel column chromatography (eluting with petroleum ether/ethyl acetate=3/1) to give the title compound (2.7 g, 77%) as a light-yellow liquid. MS (ESI): 421 m/z [M+Na]⁺, retention time: 2.32 minutes, purity: 95% (214 nm) (LC-MS method 2).

The following intermediates were prepared based on the procedures described for Intermediate 33B.

| Inter. | | | |
|---|---|---|---|
| No. | Structure | Name | MS m/z [M + H]⁺ |
| 33B-1 | 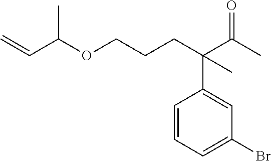 [SMILES: C=CC(C)OCCCC(C)(C(C)=O)c1cccc(Br)c1] | 3-(3-bromophenyl)-6- (but-3-en-2-yloxy)-3- methylhexan-2-one | MS: 339, 341 m/z[M + H]⁺; ¹H NMR (400 MHz, CDCl₃) δ 7.41-7.36 (m, 2H), 7.21 (t, J = 8.0 Hz, 1H), 7.13 (d, J = 7.6 Hz, 1H), 5.73-5.67 (m, 1H), 5.21-5.04 (m, 2H), 3.80-3.73 (m, 1H), 3.45-3.36 (m, 1H), 3.30- 3.23 (m, 1H), 1.99-1.89 (m, 5H), 1.46 (s, 3H), 1.43- 1.24 (m, 2H), 1.21 (d, J = 6.4 Hz, 3H) ppm. |
| 33B-2 | 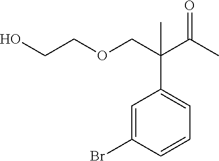 [SMILES: CC(=O)C(C)(COCCO)c1cccc(Br)c1] | 3-(3-bromophenyl)-4- (2-hydroxyethoxy)-3- methylbutan-2-one | 301, 303 |

Intermediate 34: 2-(5-(3-Cyano-4-fluorophenoxy)-6-fluoro-1-tosyl-1H-indol-4-yl)acetic acid

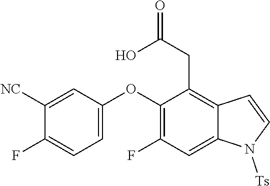 [SMILES: Cc1ccc(S(=O)(=O)n2ccc3c(CC(=O)O)c(Oc4ccc(F)c(C#N)c4)c(F)cc32)cc1]

To a stirred solution of ethyl 2-(5-(3-cyano-4-fluorophenoxy)-6-fluoro-l-tosyl-1H-indol-4-yl)acetate (Intermediate 29A, 4.2 g, 8.24 mmol) in tetrahydrofuran (20 mL) and water (20 mL) was added lithium hydroxide monohydrate (3.45 g, 82.4 mmol). The mixture was stirred overnight and concentrated. The residue was diluted with 20 mL of water, acidified with 1 M hydrochloric acid to pH ˜2. The formed precipitate was collected by filtration to give the title compound (3.9 g, 98%) as a yellow solid. MS (ESI): 483 m/z [M+H]⁺, retention time: 2.06 minutes, purity: 90% (254 nm) (LC-MS method 6).

Intermediate 35: 2-Fluoro-5-((6-fluoro-4-((2-iodothiazol-4-yl)methyl)-1-tosyl-1H-indol-5-yl)oxy)benzonitrile

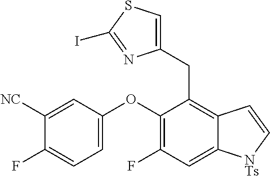 [SMILES: Cc1ccc(S(=O)(=O)n2ccc3c(Cc4csc(I)n4)c(Oc4ccc(F)c(C#N)c4)c(F)cc32)cc1]

To a solution of 5-((4-((2-aminothiazol-4-yl)methyl)-6-fluoro-1-tosyl-1H-indol-5-yl)oxy)-2-fluorobenzonitrile (Intermediate 35A, 0.700 g, 0.00130 mol) in acetonitrile (25 mL) was added copper (I) iodide (0.200 g, 0.00183 mol) and tert-butyl nitrite (0.188 g, 0.00183 mol). The reaction was stirred at 60° C. for 3 hours and concentrated. The residue was purified by automated silica gel column chromatography (12 g column, petroleum ether ethyl acetate=4:1 to 1:1) to give the title compound (0.448 g, 53.0%) as a white solid. MS (ESI): 648 m/z [M+H]⁺, retention time: 2.31 minutes, purity: 95% (254 nm) (LC-MS method 6).

Intermediate 35A: 5-((4-((2-Aminothiazol-4-yl)methyl)-6-fluoro-1-tosyl-1H-indol-5-yl)oxy)-2-fluorobenzonitrile

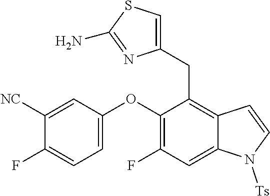 [SMILES: Cc1ccc(S(=O)(=O)n2ccc3c(Cc4csc(N)n4)c(Oc4ccc(F)c(C#N)c4)c(F)cc32)cc1]

To a stirred solution of 5-((4-(3-bromo-2-oxopropyl)-6-fluoro-1-tosyl-1H-indol-5-yl)oxy)-2-fluorobenzonitrile (Intermediate 2-24, 2.7 g, 4.83 mmol) in ethanol (20 mL) was added thiourea (1.10 g, 14.5 mmol). The mixture was refluxed for 3 hours and concentrated, and the residue was partitioned between water (50 mL) and dichloromethane (50 mL). The separated organic layer, combined with two additional dichloromethane extracts, was dried over sodium sulfate, and concentrated to give the crude title compound (2.3 g, 80%) as yellow solid. The crude product was used for the next step without further purification. MS (ESI): 537 m/z [M+H]⁺, retention time: 2.00 minutes, purity: 90% (254 nm) (LC-MS method 6). ¹HNMR (400 MHz, CD₃OD) δ 7.78-7.71 (m, 3H), 7.34 (d, J=3.6 Hz, 1H), 7.28 (d, J=8.0 Hz, 2H), 7.09 (t, J=8.8 Hz, 1H), 7.01-6.92 (m, 2H), 6.73-6.72 (m, 1H), 6.71 (s, 1H), 3.89 (s, 2H), 2.28 (s, 3H) ppm.

Intermediate 36: 7-Bromo-2-(3-bromo-2-fluorophenyl)-5,5-dimethylheptanoic acid

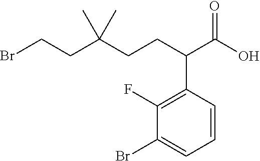 [SMILES: CC(C)(CCBr)CCC(C(=O)O)c1cccc(Br)c1F]

To a stirred solution of 2-(3-bromo-2-fluorophenyl)-5,5-dimethyl-7-(tosyloxy)heptanoic acid (Intermediate 36A, 3.6 g, 7.18 mmol) in 50 mL of acetone was added lithium bromide (2.5 g, 28.7 mmol). The mixture was stirred at 75° C. for four hours and concentrated. The residue was partitioned between water (40 mL) and ethyl acetate (50 mL). The separated organic layer, combined with two additional ethyl acetate extracts (2×30 mL), was washed with water, brine (10 mL), dried over sodium sulfate, and concentrated to give the title compound (1.71 g, 29% for 2 steps). MS (ESI): 411 m/z [M+H]⁺, retention time: 2.29 minutes, purity: 57% (214 nm) (LC-MS method 2).

Intermediate 36A: 2-(3-Bromo-2-fluorophenyl)-5,5-dimethyl-7-(tosyloxy)heptanoic acid

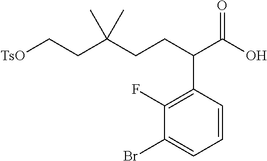 [SMILES: Cc1ccc(S(=O)(=O)OCCC(C)(C)CCC(C(=O)O)c2cccc(Br)c2F)cc1]

To a stirred and cooled (−78° C.) solution of diisopropylamine (2.777 g, 27.5 mmol) in 40 mL of tetrahydrofuran was added n-butyl lithium (2.5 M in tetrahydrofuran, 11 mL, 27.5 mmol). The reaction was stirred at this temperature for 30 minutes, treated with a solution of 2-(3-bromo-2-fluorophenyl)acetic acid (2.912 g, 12.5 mmol) in 10 mL tetrahydrofuran, and stirred at the same temperature for 30 minutes. To this solution was added hexamethylphosphoramide (27.5 mmol, 4.928 g) and stirred for another 30 minutes, followed by addition of 3,3-dimethylpentane-1,5-diyl bis(4-methylbenzenesulfonate) (11 g, 25 mmol) in one portion. The mixture was warmed to room temperature and stirred for 16 hours, quenched with a mixture of 1 M hydrochloric acid (10 mL) and saturated ammonium chloride (10 mL), and extracted with ethyl acetate (3×50 mL). The combined organic phases were washed with brine, dried over sodium sulfate, and concentrated. The residue was purified by automated flash chromatography (eluting with 10-30% ethyl acetate in petroleum ether) to give the title compound (3.7 g, 60%). MS (ESI): 518, 520 m/z [M+NH₄]⁺, retention time: 1.47 minutes, purity: 19% (214 nm) (LC-MS method 7).

The following intermediate was prepared based on the procedures described for Intermediate 36A.

| Inter. | | | MS m/z |
|---|---|---|---|
| No. | Structure | Name | [M + H]⁺ |
| 36A-1 | 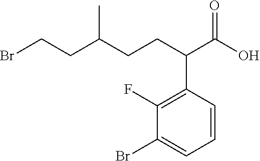 [SMILES: CC(CCBr)CCC(C(=O)O)c1cccc(Br)c1F] | 7-bromo-2-(3-bromo- 2-fluorophenyl)-5- methylheptanoic acid | 419 [M + Na]⁺ |

Intermediate 37: 5-((4-((4-Bromo-1H-pyrazol-1-yl)methyl)-6-fluoro-1H-indol-5-yl)oxy)-2-fluorobenzonitrile

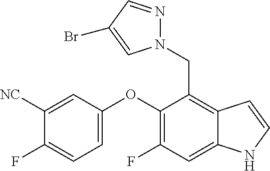 [SMILES: N#Cc1cc(Oc2c(F)cc3[nH]ccc3c2Cn2cc(Br)cn2)ccc1F]

To a stirred solution of 5-((4-((4-bromo-1H-pyrazol-1-yl)methyl)-6-fluoro-1-(phenylsulfonyl)-1H-indol-5-yl)oxy)-2-fluorobenzonitrile (Intermediate 37A, 1.2 g, 2.11 mmol) in methanol (10 mL) was added potassium carbonate (873 mg, 6.32 mmol). The reaction was heated to 60° C. for 8 hours and concentrated. The residue was partitioned between water (50 mL) and ethyl acetate (50 mL). The separated organic layer, combined with two additional ethyl acetate extracts (2×50 mL), was washed with brine, dried over magnesium sulfate, and concentrated. The residue was purified by preparative thin layer chromatography to give the title compound (0.7 g, 77%) as a white solid. MS (ESI): 529, 531 m/z [M+H]⁺, retention time: 2.02 minutes, purity: 90% (214 nm) (LC-MS method 2).

Intermediate 37A: 5-((4-((4-Bromo-1H-pyrazol-1-yl)methyl)-6-fluoro-1-(phenylsulfonyl)-1H-indol-5-yl)oxy)-2-fluorobenzonitrile

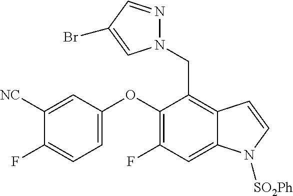

To a stirred solution of 5-((4-(bromomethyl)-6-fluoro-1-(phenylsulfonyl)-1H-indol-5-yl)oxy)-2-fluorobenzonitrile (Intermediate 8, 1.5 g, 2.98 mmol) in 5 mL of dimethylformamide was added 4-bromo-1H-pyrazole (438 mg, 2.98 mmol) and potassium carbonate (0.61 g, 4.47 mmol). The reaction was stirred at 80° C. for 18 hours, cooled to room temperature and diluted with water (50 mL). The mixture was extracted with ethyl acetate (3×50 mL). The organic phases were dried over magnesium sulfate and concentrated. The residue was purified by automated flash chromatography (50 g silica gel column, eluting with 20% ethyl acetate/petroleum ether) to provide the title compound (1.2 g, 70%) as yellow oil. MS (ESI): 569, 571 m/z [M+H]⁺, retention time: 1.91 minutes, purity: 92% (254 nm) (LC-MS method 7).

Intermediate 38: Tert-Butyl((6-iodo-2-methylhexyl)oxy)dimethylsilane

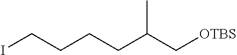 [SMILES: CC(CCCCI)CO[Si](C)(C)C(C)(C)C]

To a stirred solution of ((6-Bromo-2-methylhexyl)oxy)(tert-butyl)dimethylsilane (Intermediate 38A, 4.7 g, 15.2 mmol) in acetone (120 mL) was added sodium iodide (6.83 g, 45.6 mmol). The reaction was stirred at 60° C. for 6 hours and concentrated. The residue was partitioned between water (100 mL) and dichloromethane (100 mL). The separated organic layer, combined with two additional dichloromethane extracts (2×50 mL), was washed with brine, dried over sodium sulfate, and concentrated. The residue was purified by automated flash chromatography (80 g silica gel column, eluting with 0-30% ethyl acetate in petroleum ether) to give the title compound (4.8 g, 88%). ¹H NMR (400 MHz, CDCl₃) δ 3.41-3.31 (m, 2H), 3.16 (t, J=7.0 Hz, 2H), 1.83-1.73 (m, 2H), 1.59-1.52 (m, 1H), 1.45-1.29 (m, 3H), 1.07-0.99 (m, 1H), 0.86 (s, 9H), 0.83 (d, J=6.7 Hz, 3H), 0.00 (s, 6H) ppm.

Intermediate 38A: ((6-Bromo-2-methylhexyl)oxy)(tert-butyl)dimethylsilane

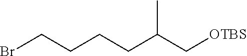 [SMILES: CC(CCCCBr)CO[Si](C)(C)C(C)(C)C]

To a stirred solution of 6-bromo-2-methylhexan-1-ol (3.4 g, 17.4 mmol) in dichloromethane (30 mL) was added imidazole (1.54 g, 22.7 mmol) and tert-butylchlorodimethylsilane (2.76 g, 18.3 mmol). The reaction was stirred at room temperature overnight, quenched with water (100 mL) and extracted with dichloromethane (3×50 mL). The combined organic phases were washed with brine, dried over sodium sulfate, and concentrated. The residue was purified by automated silica gel column chromatography (80 g column, eluting with 0˜25% ethyl acetate in petroleum ether) to give the title compound (4.7 g, 87%) as a oil. ¹H NMR (400 MHz, CDCl₃) δ 3.42-3.31 (m, 4H), 1.86-1.76 (m, 2H), 1.59-1.54 (m, 1H), 1.48-1.34 (m, 3H), 1.09-1.00 (m, 1H), 0.86 (s, 9H), 0.83 (d, J=6.7 Hz, 3H), 0.00 (s, 6H) ppm.

Intermediate 39: 2-(3-Iodophenyl)-2,7,7-trimethylnon-8-ynehydrazide

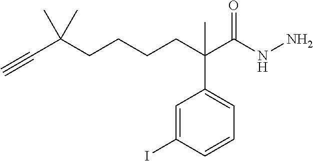 [SMILES: C#CC(C)(C)CCCCC(C)(C(=O)NN)c1cccc(I)c1]

A solution of 2-(3-iodophenyl)-2,7,7-trimethylnon-8-ynoic acid (Intermediate 7-11, 5.6 g, 14.1 mmol) in thionyl chloride (30 mL) was refluxed for one hour and concentrated. The residue was dissolved in acetonitrile (50 mL), added to a stirred and cooled (0° C.) solution of hydrazine hydrate (7.03 g, 141 mmol) in acetonitrile (100 mL) dropwise. The reaction was stirred for one additional hour at this temperature, quenched with water (100 mL), and extracted with ethyl acetate (3×30 mL). The combined organic extracts were dried over magnesium sulfate and concentrated. The crude product was purified by automated flash chromatography (80 g silica gel column, eluting with 0-10% methanol in dichloromethane) to give the title compound (5.17 g, 89%) as oil. MS (ESI): 413 m/z [M+H]⁺, retention time: 2.07 minutes, purity: >99% (214 nm) (LC-MS method 2).

Intermediate 40: Methyl 2-(3-iodophenyl)-2-methyl-7-(3-methyl-2-oxooxazolidin-5-yl)heptanoate

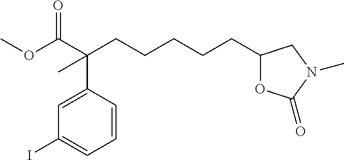

To a stirred and cooled (0° C.) solution of methyl 2-(3-iodophenyl)-2-methyl-7-(2-oxooxazolidin-5-yl)heptanoate (Intermediate 40D, 4.6 g, 10.3 mmol) in dichloromethane (93 mL) was added sodium hydride (828 mg, 20.6 mmol). The reaction was stirred for 1 hour, then treated with iodomethane (1.92 mL, 30.9 mmol). The mixture was stirred at room temperature overnight, diluted with ethyl acetate (600 mL). The solution was washed with water, brine, dried over sodium sulfate, and concentrated and the residue purified by silica gel column chromatography (80 g silica gel column, eluting with 0-100% ethyl acetate in petroleum ether) to give the title compound (4.39 g, 93%) as a colorless oil. MS (ESI): 460 m/z [M+H]⁺, retention time: 2.20 minutes, purity: 84% (254 nm) (LC-MS method 5).

Intermediate 40A: Methyl 9-bromo-8-hydroxy-2-(3-iodophenyl)-2-methylnonanoate

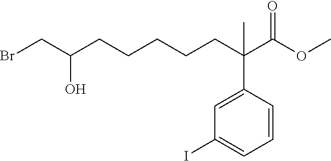 [SMILES: COC(=O)C(C)(CCCCCC(O)CBr)c1cccc(I)c1]

To a stirred and cooled (0° C.) solution of methyl 2-(3-iodophenyl)-2-methylnon-8-enoate (Intermediate 7A-16, 20.38 g, 52.8 mol) in dimethyl sulfoxide (57 mL) was added N-bromosuccinimide (10.34 g, 58.1 mmol) and water (1.9 mL, 106 mmol) slowly. The reaction was stirred for 2 hours, then diluted with ethyl acetate. The solution was washed with water, brine, dried with sodium sulfate, and concentrated. The residue was purified by automated silica gel column chromatography (330 g silica gel column, eluting with 0-30% ethyl acetate in petroleum ether) to give the title compound (15.7 g, 62%) as a colorless oil. MS (ESI): 483, 485 m/z [M+H]⁺

Intermediate 40B: Methyl 9-azido-8-hydroxy-2-(3-iodophenyl)-2-methylnonanoate

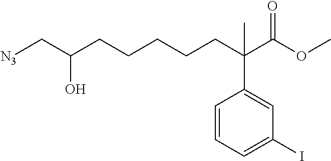 [SMILES: COC(=O)C(C)(CCCCCC(O)CN=[N+]=[N-])c1cccc(I)c1]

To a solution of methyl 9-bromo-8-hydroxy-2-(3-iodophenyl)-2-methylnonanoate (Intermediate 40A, 15.7 g, 32.7 mmol) in dry dimethylformamide (97 mL) was added sodium azide (4.2 g, 65.3 mmol), the mixture was stirred at 90° C. overnight, cooled to room temperature and diluted with ethyl acetate (200 mL). The solution was washed with water, brine, dried over sodium sulfate, and concentrated. The residue was purified by silica gel column chromatography (80 g silica gel column, eluting with 0-30% ethyl acetate in petroleum ether) to give the title compound (11.3 g, 78%) as a colorless oil. MS (ESI): 468 m/z [M+Na]⁺, retention time: 2.16 minutes, purity: 83% (214 nm) (LC-MS method 2).

Intermediate 40C: Methyl 9-amino-8-hydroxy-2-(3-iodophenyl)-2-methylnonanoate

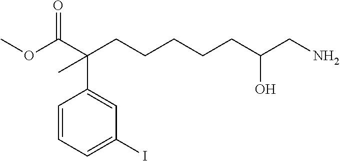 [SMILES: COC(=O)C(C)(CCCCCC(O)CN)c1cccc(I)c1]

To a solution of methyl 9-azido-8-hydroxy-2-(3-iodophenyl)-2-methylnonanoate (Intermediate 40B, 6.2 g, 13.9 mmol) in tetrahydrofuran-water (5:1, 62.7 mL) was added PPh₃ (4.0 g, 15.3 mmol). The solution was stirred at 50° C. overnight, then diluted with ethyl acetate (500 mL). The mixture was washed with water, brine, dried over sodium sulfate, and concentrated. The residue was purified by silica gel column chromatography (80 g silica gel column, eluting with 0-10% methanol in dichloromethane) to give the title compound (4.64 g, 80%) as a colorless oil. MS (ESI): 420 m/z [M+H]⁺, retention time: 1.47 minutes, purity: 83% (214 nm) (LC-MS method 5).

Intermediate 40D: Methyl 2-(3-iodophenyl)-2-methyl-7-(2-oxooxazolidin-5-yl)heptanoate

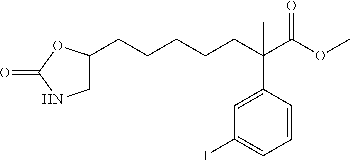 [SMILES: COC(=O)C(C)(CCCCCC1CNC(=O)O1)c1cccc(I)c1]

To a stirred solution of methyl 9-amino-8-hydroxy-2-(3-iodophenyl)-2-methylnonanoate (Intermediate 40C, 4.64 g, 11.1 mmol) in dichloromethane (124 mL) was added 1,1′-carbonyldiimidazole (1.88 g, 11.6 mmol) and imidazole (377 mg, 5.55 mmol). The reaction was stirred at room temperature overnight, diluted with ethyl acetate (500 mL). The solution was washed with water, brine, dried over sodium sulfate, and concentrated. The residue was purified by silica gel column chromatography (40 g silica gel column, eluting with 0-10% methanol in dichloromethane) to give the title compound (4.6 g, 93%) as a colorless oil. MS (ESI): 446 m/z [M+H]⁺, retention time: 2.11 minutes, purity: 84% (214 nm) (LC-MS method 5).

Intermediate 41: 7-Iodo-4,4-dimethylhept-1-yne

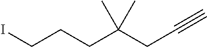

To a stirred and cooled (0° C.) solution of triphenyl phosphine (14.8 g, 56.6 mmol) and imidazole (2.96 g, 56.6 mmol) in dichloromethane (40 mL) was added iodine (14.4 g, 56.6 mmol). The mixture was stirred for 10 minutes at this temperature, then treated with 4,4-dimethylhept-6-yn-1-ol (6.1 g, 43.5 mmol), and stirred at room temperature for another 1 hour. The reaction mixture was concentrated. The residue was purified by column chromatography (petroleum ether/ethyl acetate=10/1) to give the title compound (7.3 g, 67%) as a yellow oil. ¹H NMR (400 MHz, CDCl₃) δ 3.17 (t, J=6.8 Hz, 2H), 2.07 (t, J=2.8 Hz, 2H), 1.99 (t, J=2.8 Hz, 1H), 1.83-1.77 (m, 2H), 1.42-1.38 (m, 2H), 0.97 (s, 6H) ppm.

Intermediate 41A: Ethyl (E)-4,4-dimethylhept-2-en-6-ynoate

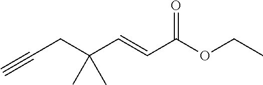

A mixture of 30% aqueous sodium hydroxide (7.77 g sodium hydroxide, 194 mmol), sodium iodide (1.46 g, 9.71 mmol), tetrabutylammonium iodide (1.79 g, 4.85 mmol) in dichloromethane (50 mL) and dimethyl sulfoxide (5 mL) was heated at 50° C. To this vigorously stirred mixture was added dropwise (over ca. 3 h) a solution of 3-bromoprop-1-yne (17.3 g, 146 mmol) and 2-methylpropanal (7.0 g, 97.1 mmol) in dichloromethane (30 mL). After stirring for 24 hours, the reaction mixture was cooled to room temperature, diluted with water (20 mL), and extracted with dichloromethane (3×20 mL). The combined organic layers were dried over magnesium sulfate and filtered. The filtrate was used as ‘is’ for the next step to avoid loss of low boiling point product.

To a stirred and cooled (0° C.) solution of ethyl 2-diethoxyphosphorylacetate (21.8 g, 97.1 mmol) in tetrahydrofuran (30 mL) was added sodium hydride (3.49 g, 87.4 mmol). The reaction was stirred at room temperature for 30 minutes, cooled to 0° C., then added the above filtrate. The mixture was stirred at room temperature for 4 hours, then quenched with brine (30 mL). The solution was extracted with dichloromethane (3×50 mL). The combined organic phases were dried over sodium sulfate and concentrated. The residue was purified by silica gel column chromatography (eluting with petroleum ether/ethyl acetate=10/1) to give the title compound (9.0 g, yield 51.4%) as yellow oil. MS (ESI): 181 m/z [M+H]⁺, retention time: 2.03 minutes, purity: >99% (214 nm) (LC-MS Method 9). ¹H NMR (400 MHz, CDCl₃) δ 6.97 (d, J=16.0 Hz, 1H), 5.81 (d, J=16.0 Hz, 1H), 4.20 (q, J=7.2 Hz, 2H), 2.24 (d, J=2.8 Hz, 2H), 2.03 (t, J=2.8 Hz, 1H), 1.30 (t, J=7.0 Hz, 3H), 1.17 (s, 6H) ppm.

Intermediate 41B: Methyl 4,4-dimethylhept-6-ynoate

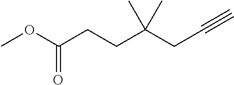

To a stirred and cooled (0° C.) solution of ethyl (E)-4,4-dimethylhept-2-en-6-ynoate (Intermediate 41A, 9.0 g, 49.9 mmol) in dry methanol (50 mL) was slowly and carefully added magnesium powder (3.64 g, 150 mmol). The mixture was stirred at room temperature overnight, then neutralized to pH ˜7 with 2 M hydrochloric acid and concentrated. The residue was partitioned between water (20 mL) dichloromethane (30 mL). The separated organic layer, combined with three additional dichloromethane extracts (3×30 mL), was dried over magnesium sulfate, and concentrated. The crude title compound (7.5 g, 89%, colorless oil) was used for the next step without further purification. ¹H NMR (400 MHz, CDCl₃) δ 3.68 (s, 3H), 2.32-2.28 (m, 2H), 2.08 (d, J=2.4 Hz, 2H), 2.00 (t, J=2.8 Hz, 1H), 1.71-1.67 (m, 2H), 0.97 (s, 6H) ppm

Intermediate 41C: 4,4-Dimethylhept-6-yn-1-ol

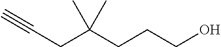

To a stirred and cooled (0° C.) suspension of lithium aluminum hydride (1.69 g, 44.6 mmol) in tetrahydrofuran (30 mL) was added methyl 4,4-dimethylhept-6-ynoate (Intermediate 41B, 7.50 g, 44.6 mmol) in tetrahydrofuran (10 mL). The mixture was stirred at 0° C. for one hour. The mixture was carefully poured into ice-water (200 g). The mixture was filtered. The filtrate was extracted with dichloromethane (3×30 mL), dried over sodium sulfate, and concentrated to give the title compound (6.4 g, 98%) as colorless oil. ¹H NMR (400 MHz, CDCl₃) δ 3.67-3.2 (m, 2H), 2.09 (d, J=2.8 Hz, 2H), 1.98 (t, J=2.8 Hz, 1H), 1.64 (s, 1H), 1.56-1.52 (m, 2H), 1.38-1.34 (m, 2H), 0.98 (s, 6H) ppm.

Intermediate 42: 2-Fluoro-5-((6-fluoro-4-((2-oxooxazolidin-5-yl)methyl)-1H-indol-5-yl)oxy)benzonitrile

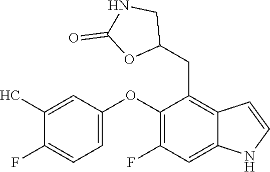 [SMILES: Cc1cc(Oc2c(F)cc3[nH]ccc3c2CC2CNC(=O)O2)ccc1F]

To a stirred solution of 2-fluoro-5-((6-fluoro-4-((2-oxooxazolidin-5-yl)methyl)-1-tosyl-1H-indol-5-yl)oxy)benzonitrile (Intermediate 42G, 0.559 g, 1.069 mmol) in methanol (6 ml) was added potassium carbonate (0.443 g, 3.207 mol). The white suspension was stirred for 4 hours at 50° C. and concentrated. The red-brownish residue was partitioned between saturated sodium bicarbonate (20 ml) and ethyl acetate (50 mL). The separated organic layer, combined with two additional ethyl acetate extracts (2×20 mL), was dried over sodium sulfate, and concentrated to afford the title compound (0.271 g, 68.7%) as a pale-yellow solid. MS (ESI): 370 m/z [M+H]⁺, retention time: 1.83 minutes, purity: >99% (254 nm) (LC-MS method 9).

Intermediate 42A: 5-((4-Allyl-6-fluoro-1H-indol-5-yl)oxy)-2-fluorobenzonitrile

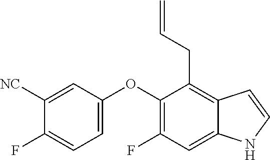 [SMILES: C=CCc1c(Oc2ccc(F)c(C#N)c2)c(F)cc2[nH]ccc12]

To a stirred and degassed solution of 5-((4-bromo-6-fluoro-1H-indol-5-yl)oxy)-2-fluorobenzonitrile (Intermediate 3, 11 g, 31.51 mmol) in N,N-dimethylformamide (110 mL) was added allyltributylstannane (12.5 g, 37.81 mmol), lithium chloride (4.0 g, 94.53 mmol) and bis(triphenylphosphine)palladium (II) dichloride (1.3 g, 1.89 mmol). The reaction was stirred at 90° C. for 5 hours, cooled to room temperature and quenched with saturated potassium fluoride (100 mL). The mixture was stirred for 10 minutes, diluted with ethyl acetate (200 mL), and filtered. The filtrate was extracted with ethyl acetate (3×200 mL). The combined organic extracts were washed with saturated lithium chloride, brine, dried over sodium sulfate and concentrated. The residue was purified by silica gel column chromatography (petroleum ether/ethyl acetate=3:1) to give the title compound (8.1 g, 83%) as a yellow solid. MS (ESI): 311 m/z [M+H]⁺, retention time: 2.10 minutes, purity: 95% (214 nm) (LC-MS method 2).

Intermediate 42B: 5-((4-Allyl-6-fluoro-1-tosyl-1H-indol-5-yl)oxy)-2-fluorobenzonitrile

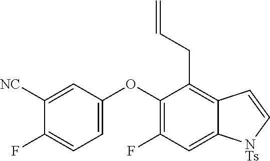 [SMILES: C=CCc1c(Oc2ccc(F)c(C#N)c2)c(F)cc2c1ccn2S(=O)(=O)c1ccc(C)cc1]

To a stirred and cooled (0° C.) solution of 5-((4-allyl-6-fluoro-1H-indol-5-yl)oxy)-2-fluorobenzonitrile (Intermediate 42A, 2 g, 0.00646 mol) in tetrahydrofuran (20 mL) was added sodium hydride (60% in mineral oil, 0.336 g, 0.00419 mol) portion wise. The reaction was stirred at room temperature for 30 minutes, then treated with a solution of tosyl chloride (1.476 g, 0.00387 mol) in tetrahydrofuran (10 mL). The mixture was warmed to room temperature and stirred for 2 additional hours, quenched with water (20 mL), and extracted with ethyl acetate (3×50 mL). The combined organic extracts were washed with brine, dried over sodium sulfate, and concentrated. The yellow solid residue was triturated with ethyl acetate/petroleum ether=1/10 (20 mL) to give the title compound (3.0 g, 100%) as a yellow solid.

The following intermediate was prepared based on the procedures described for Intermediate 42B

| Inter. | | | MS m/z |
|---|---|---|---|
| No. | Structure | Name | [M + H]⁺ |
| 42B-1 | 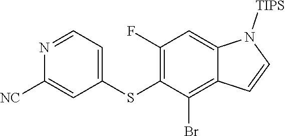 | 4-((4-bromo-6-fluoro-1-tosyl-1H-indol-5- yl)thio)picolinonitrile | 502, 504 |
| 42B-2 | 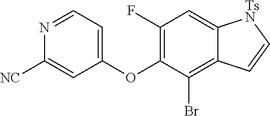 [SMILES: Cc1ccc(S(=O)(=O)n2ccc3c(Br)c(Oc4ccnc(C#N)c4)c(F)cc32)cc1] | 4-((4-bromo-6-fluoro-1-tosyl-1H-indol-5- yl)oxy)picolinonitrile | 486, 488 |
| 42B-3 | 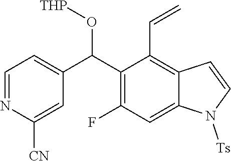 | 4-((6-fluoro-1-tosyl-4-vinyl-1H-indol-5- yl)((tetrahydro-2H-pyran-2- yl)oxy)methyl)picolinonitrile | 532 m/z [M + H]⁺; RT: 2.10 + 2.14 minutes (LC-MS method 26) |
| 42B-4 | 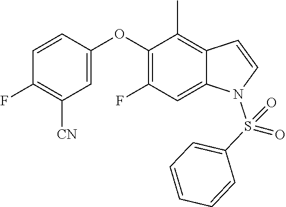 [SMILES: Cc1c(Oc2ccc(F)c(C#N)c2)c(F)cc2c1ccn2S(=O)(=O)c1ccccc1] | 2-fluoro-5-((6-fluoro-4-methyl-1- (phenylsulfonyl)-1H-indol-5-yl)oxy)benzonitrile | 425 |

Intermediate 42C: 5-((4-(2,3-Dihydroxypropyl)-6-fluoro-1-tosyl-1H-indol-5-yl)oxy)-2-fluorobenzonitrile

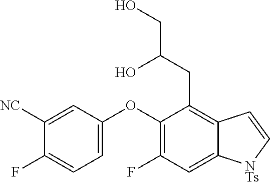 [SMILES: Cc1ccc(S(=O)(=O)n2ccc3c(CC(O)CO)c(Oc4ccc(F)c(C#N)c4)c(F)cc32)cc1]

To a stirred solution of 5-((4-allyl-6-fluoro-1-tosyl-1H-indol-5-yl)oxy)-2-fluorobenzonitrile (Intermediate 42B, 5 g, 10.78 mmol) in tetrahydrofuran (50 mL) was added N-methylmorpholine N-oxide (2.53 g, 21.55 mmol) osmium tetroxide (4% in water, 2 drops). The mixture was stirred at room temperature overnight, quenched with aqueous sodium dithionite (20 mL), and then extracted with ethyl acetate (3×50 mL). The combined organic layers were washed with aqueous sodium dithionite (20 mL), dried over sodium sulfate and concentrated. The yellow oily residue was purified by automated flash chromatography (40 g silica gel column, eluting with 0-80% ethyl acetate in petroleum ether) to give the title compound as white solid (4.783 g, 89%). MS (ESI): 499 m/z [M+H]⁺, retention time: 1.73 minutes, purity: >99% (214 nm) (LC-MS method 2).

Intermediate 42D: 3-(5-(3-Cyano-4-fluorophenoxy)-6-fluoro-1-tosyl-1H-indol-4-y)-2-hydroxypropyl methanesulfonate

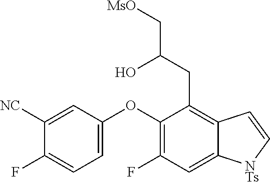 [SMILES: Cc1ccc(S(=O)(=O)n2ccc3c(CC(O)COS(C)(=O)=O)c(Oc4ccc(F)c(C#N)c4)c(F)cc32)cc1]

To a stirred and cooled (0° C.) solution of 5-[4-(2,3-dihydroxypropyl)-6-fluoro-l-(p-tolylsulfonyl)-indol-5-yl]oxy-2-fluoro-benzonitrile (Intermediate 42C, 3 g, 6.024 mmol) and triethylamine (0.914 g, 9.036 mmol) in dichloromethane (30 mL) was added methanesulfonyl chloride (0.76 g, 6.6 mmol) dropwise. The pale-yellow suspension mixture was stirred for 30 minutes at room temperature and diluted with 50 mL of dichloromethane. The red-brownish solution was washed with water, saturated sodium bicarbonate (20 mL), dried over sodium sulfate, and concentrated to afford the crude title compound (4.6 g, crude) as a brown oil. MS (ESI): 577 m/z [M+H]⁺, retention time: 1.79 minutes (LC-MS method 2).

Intermediate 42E: 5-((4-(3-Azido-2-hydroxypropyl)-6-fluoro-1-tosyl-1H-indol-5-yl)oxy)-2-fluorobenzonitrile

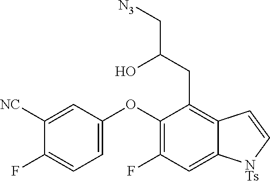 [SMILES: Cc1ccc(S(=O)(=O)n2ccc3c(CC(O)CN=[N+]=[N-])c(Oc4ccc(F)c(C#N)c4)c(F)cc32)cc1]

To a stirred solution of 3-(5-(3-cyano-4-fluorophenoxy)-6-fluoro-1-tosyl-1H-indol-4-yl)-2-hydroxypropyl methanesulfonate (Intermediate 42D, 4.63 g, 8.03 mmol) in N,N-dimethylformamide (46 mL) was added sodium azide (2.61 g, 40.16 mmol). The yellow mixture was stirred for two hours at 80° C., cooled to room temperature and quenched with water (60 mL). The mixture was extracted with ethyl acetate (3×50 mL). The combined organic phases were washed with saturated lithium chloride, brine, dried over sodium sulfate, and concentrated. The residue was purified by automated flash chromatography (80 g silica gel column, eluting with 30-60% ethyl acetate in petroleum ether) to afford the desired product (1.564 g, 53% for two steps) as yellow solid. MS (ESI): 524 m/z [M+H]⁺, retention time: 1.87 minutes (LC-MS method 7).

Intermediate 42F: 5-((4-(3-Amino-2-hydroxypropyl)-6-fluoro-1-tosyl-1H-indol-5-yl)oxy)-2-fluorobenzonitrile

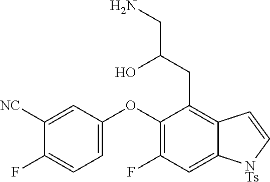 [SMILES: Cc1ccc(S(=O)(=O)n2ccc3c(CC(O)CN)c(Oc4ccc(F)c(C#N)c4)c(F)cc32)cc1]

To a stirred solution of 5-((4-(3-azido-2-hydroxypropyl)-6-fluoro-1-tosyl-1H-indol-5-yl)oxy)-2-fluorobenzonitrile (Intermediate 42E, 3 g, 5.74 mmol) in tetrahydrofuran (30 mL) was added palladium on carbon (10%, 50% wet, 600 mg). The mixture was stirred under hydrogen balloon overnight, then filtered through a pad of Celite and washed the filter cake with ethyl acetate (2×20 mL). The filtrate was concentrated to give the title compound (2.9 g, 100%) as a brown oil. MS (ESI): 498 m/z [M+H]⁺, retention time: 1.85 minutes (LC-MS method 7).

Intermediate 42G: 2-Fluoro-5-((6-fluoro-4-((2-oxooxazolidin-5-yl)methyl)-1-tosyl-1H-indol-5-yl)oxy)benzonitrile

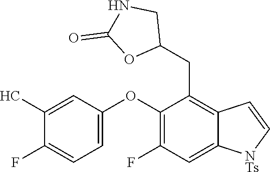 [SMILES: Cc1ccc(S(=O)(=O)n2ccc3c(CC4CNC(=O)O4)c(Oc4ccc(F)c(C)c4)c(F)cc32)cc1]

To a stirred solution of 5-[4-(3-amino-2-hydroxy-propyl)-6-fluoro-l-(p-tolylsulfonyl)indol-5-yl]oxy-2-fluoro-benzonitrile (Intermediate 42F, 3 g, 6.036 mmol) in dichloromethane (60 ml) was added 1,1′-carbonyldiimidazole (1.028 g, 6.338 mmol) and imidazole (0.205 g, 3.018 mmol). The dark solution was stirred at room temperature overnight and quenched with water (50 mL). The mixture was extracted with ethyl acetate (3×50 mL). The combined organic phases were washed with brine (20 mL), dried over sodium sulfate, filtered and concentrated. The residue was purified by automated flash chromatography (80 g silica gel column, eluting with 60-80% ethyl acetate in petroleum ether) to give the title compound (2.03 g, 57%) as a yellow oil. MS (ESI): 524 m/z [M+H]⁺, retention time: 2.06 minutes (LC-MS method 7).

Intermediate 43: 2-Fluoro-5-((6-fluoro-4-((4-iodo-1H-imidazol-1-yl)methyl)-1H-indol-5-yl)oxy)benzonitrile

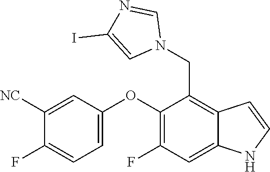 [SMILES: N#Cc1cc(Oc2c(F)cc3[nH]ccc3c2Cn2cnc(I)c2)ccc1F]

To a solution of 2-fluoro-5-((6-fluoro-4-((4-iodo-1H-imidazol-1-yl)methyl)-1-(phenylsulfonyl)-1H-indol-5-yl)oxy)benzonitrile (Intermediate 43A, 3.6 g, 5.84 mmol) in dioxane (60 mL) was added sodium hydroxide (0.7 g, 17.5 mmol) in water (6 mL). The reaction was stirred at room temperature overnight, then diluted with water (80 mL). The mixture was extracted with ethyl acetate (3×80 mL). The combined organic extracts were washed with water, brine, dried over sodium sulfate and concentrated. The residue was purified by column chromatography (40 g; eluting with ethyl acetate in petroleum ether from 0 to 35%) to give the title compound (2.0 g; 62%) as solid. MS (ESI): 477 m/z [M+H]⁺, retention time: 1.89 minutes; purity: 86% (254 nm) (LC-MS method 2).

Intermediate 43A: 2-Fluoro-5-((6-fluoro-4-((4-iodo-1H-imidazol-1-yl)methyl)-1-(phenylsulfonyl)-1H-indol-5-yl)oxy)benzonitrile

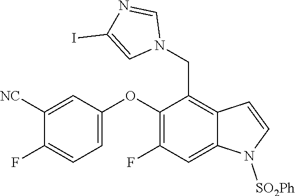

To a stirred solution of 4-iodo-1H-imidazole (1.30 g, 6.7 mmol) and 5-((4-(bromomethyl)-6-fluoro-1-(phenylsulfonyl)-1H-indol-5-yl)oxy)-2-fluorobenzonitrile (Intermediate 8, 3.37 g, 6.7 mmol) in N,N-dimethylformamide (60 mL) was added potassium carbonate (1.85 g, 13.4 mmol). The mixture was stirred at room temperature for 16 hours, then diluted with water (100 mL). The solution was extracted with ethyl acetate (3×80 mL). The combined organic extracts were washed with water, brine, dried over sodium sulfate and concentrated. The residue was purified by automated column chromatography (40 g; eluting with ethyl acetate in petroleum ether from 0 to 75% to give the title compound (3.7 g; 90%) as solid. MS (ESI): 617 m/z [M+H]⁺, retention time: 2.06 minutes; purity: 79% (254 nm) (LC-MS method 7).

Intermediate 44: 2-Fluoro-5-((6-fluoro-4-((3-methyl-2-oxooxazolidin-5-yl)methyl)-1H-indol-5-yl)oxy)benzonitrile

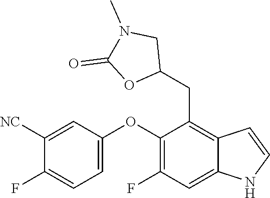 [SMILES: CN1CC(Cc2c(Oc3ccc(F)c(C#N)c3)c(F)cc3[nH]ccc23)OC1=O]

To a stirred solution of 2-fluoro-5-((6-fluoro-4-((3-methyl-2-oxooxazolidin-5-yl)methyl)-1-(triisopropylsilyl)-1H-indol-5-yl)oxy)benzonitrile (Intermediate 44D, 1.62 g, 3 mmol) in methanol (6 mL) and tetrahydrofuran (18 mL) was added lithium hydroxide monohydrate (0.38 g; 9 mmol) in water (6 mL). The reaction was stirred at 35° C. for 16 hours, diluted with ethyl acetate (100 mL). The solution was washed with water, brine, dried over sodium sulfate, and concentrated. The residue was purified by automated silica gel column (40 g column, eluting with 0-100% ethyl acetate in petroleum ether) to give the title compound (0.75 g, 65%) as a white solid. MS (ESI): 384 m/z [M+H]⁺, retention time: 1.58 minutes; purity: >99% (254 nm) (LC-MS method 2).

Intermediate 44A: 5-((4-Allyl-6-fluoro-1-(triisopropylsilyl)-1H-indol-5-yl)oxy)-2-fluorobenzonitrile

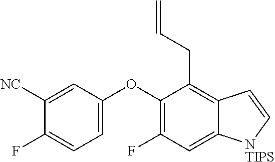

To a stirred and cooled (0° C.) solution of 5-((4-allyl-6-fluoro-1H-indol-5-yl)oxy)-2-fluorobenzonitrile (Intermediate 42A, 5.58 g, 18 mmol) in tetrahydrofuran (150 mL) was added sodium hydride (60% in mineral oil, 0.86 g, 21.6 mmol). The reaction was stirred at room temperature for 0.5 hours, then treated with triisopropylsilyl chloride (4.16 g, 21.6 mmol). The mixture was warmed to room temperature and stirred for four hours, quenched with saturated ammonium chloride. The solution was extracted with ethyl acetate (2×150 mL). The combined organic extracts were washed with brine, dried over sodium sulfate, and concentrated. The residue was purified by flash chromatography (80 g silica gel column, eluting with 0-20% ethyl acetate in petroleum ether) to give the title compound (7.05 g, 83%) as a solid. MS (ESI): 467 m/z [M+H]⁺, retention time: 2.69 minutes; purity: >99% (254 nm) (LC-MS method 2).

Intermediate 44B: Benzyl (3-(5-(3-cyano-4-fluorophenoxy)-6-fluoro-1-(triisopropylsilyl)-1H-indol-4-yl)-2-hydroxypropyl)carbamate

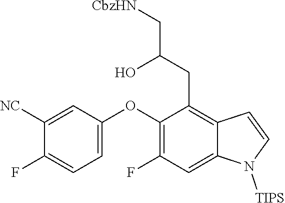

To a stirred solution of benzyl ((4-chlorobenzoyl)oxy)carbamate (1.03 g, 3.4 mmol) in acetonitrile (24 mL) was added osmium tetroxide (24 mg, 0.16 mmol). The mixture was stirred at room temperature for 10 minutes and then treated with a solution of 5-((4-allyl-6-fluoro-1-(triisopropylsilyl)-1H-indol-5-yl)oxy)-2-fluorobenzonitrile (Intermediate 44A, 1.12 g, 2.4 mmol) in acetonitrile (24 mL), followed by the addition of water (5 mL). The reaction was stirred at room temperature for 16 hours, quenched with saturated sodium thiosulfate (50 mL). The mixture was stirred for another 5 minutes, then partitioned between water (150 mL) and ethyl acetate (100 mL). The separated organic layer, combined with two additional ethyl acetate extracts (2×50 mL), was washed with saturated sodium bicarbonate, brine, dried over sodium sulfate, and concentrated. The residue was purified by automated flash chromatography (40 g silica gel column; eluting with 0% to 60% ethyl acetate/petroleum ether) to give the title compound (1.11 g, 72%) as a solid. MS (ESI): 634 m/z [M+H]⁺, retention time: 2.41 minutes; purity: >99% (254 nm) (LC-MS method 2).

Intermediate 44C: 2-Fluoro-5-((6-fluoro-4-((2-oxooxazolidin-5-yl)methyl)-1-(triisopropylsilyl)-1H-indol-5-yl)oxy)benzonitrile

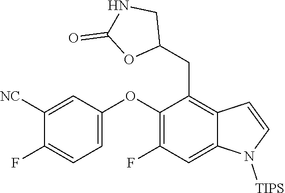

To a stirred and cooled (0° C.) solution of benzyl (3-(5-(3-cyano-4-fluorophenoxy)-6-fluoro-1-(triisopropylsilyl)-1H-indol-4-yl)-2-hydroxypropyl)carbamate (Intermediate 44B, 0.51 g, 0.8 mmol) in tetrahydrofuran (15 mL) was added sodium hydride (60% in mineral oil, 0.1 g, 2.4 mmol). The mixture was stirred at room temperature overnight, quenched with saturated ammonium chloride (30 mL). The solution was extracted with ethyl acetate (3×50 mL). The combined organic extracts were washed with brine, dried over sodium sulfate, and concentrated. The residue was purified by automated flash chromatography (20 g silica gel column, eluting with 0-70% ethyl acetate in petroleum ether) to give the title compound (0.17 g, 42%) as a solid. MS (ESI): 526 m/z [M+H]⁺, retention time: 2.44 minutes; purity: >99% (254 nm) (LC-MS method 2).

Intermediate 44D: 2-Fluoro-5-((6-fluoro-4-((3-methyl-2-oxooxazolidin-5-yl)methyl)-1-(triisopropylsilyl)-1H-indol-5-yl)oxy)benzonitrile

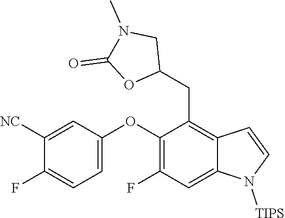

To a stirred and cooled (0° C.) solution of 2-fluoro-5-((6-fluoro-4-((2-oxooxazolidin-5-yl)methyl)-1-(triisopropylsilyl)-1H-indol-5-yl)oxy)benzonitrile (Intermediate 44C, 0.17 g, 0.33 mmol) in tetrahydrofuran (10 mL) was added sodium hydride (60% in mineral oil, 16 mg, 0.4 mmol). The mixture was stirred at room temperature for 0.5 hours, then treated with iodomethane (52 mg, 0.36 mmol). The mixture was stirred for 3 hours and quenched with saturated ammonium chloride (30 mL). The solution was extracted with ethyl acetate (3×30 mL). The combined extracts were washed with brine, dried over sodium sulfate, and concentrated. The residue was purified by automated flash chromatography (20 g silica gel column, eluting with 0-60% ethyl acetate in petroleum ether) to give the title compound (0.13 g, 75%) as a solid. MS (ESI): 540 m/z [M+H]⁺, retention time: 2.35 minutes; purity: >99% (254 nm) (LC-MS method 2).

Intermediate 45: 2-(3-Bromophenyl)-2-methyl-5-((2-methylbut-3-yn-2-yl)oxy)pentanehydrazide

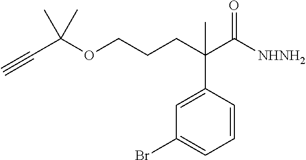 [SMILES: C#CC(C)(C)OCCCC(C)(C(=O)NN)c1cccc(Br)c1]

To a stirred solution of 2-(3-bromophenyl)-2-methyl-5-((2-methylbut-3-yn-2-yl)oxy)pentanoic acid (Intermediate 1-5, 1.5 g, 3.78 mmol) in N,N-dimethylformamide (50 mL) was added 1-hydroxybenzotriazole (0.613 g, 4.54 mmol), 1-ethyl-3-(3-dimethylaminopropyl)carbodiimide hydrochloride (0.869 g, 4.54 mmol). The mixture was stirred at room temperature for 2 hours, cooled to 0° C., and treated with hydrazine hydrate (0.167 mL, 7.56 mmol) dropwise. The mixture was stirred at room temperature for 1 hour and concentrated. The residue was diluted with ethyl acetate (100 mL), washed with water, brine, dried over sodium sulfate, and concentrated. The crude product was purified by automated flash chromatography (20 g silica gel column, eluting with 0-40% methanol in dichloromethane) to give the title compound (1.4 g, 89%) as an oil. MS (ESI): 367, 369 m/z [M+H]⁺, retention time: 1.91 minutes; purity: >99% (254 nm) (LC-MS method 9).

Intermediate 46: 2-(3-Iodophenyl-2-methyldec-8-ynoic acid

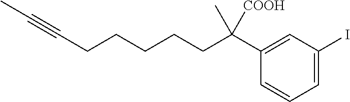 [SMILES: CC#CCCCCCC(C)(C(=O)O)c1cccc(I)c1]

To a stirred and cooled (−78° C.) solution of 2-(3-iodophenyl)-2-methylnon-8-ynoic acid (Intermediate 7-6, 4 g, 10.8 mmol) in 40 mL of tetrahydrofuran was added lithium diisopropylamide (27 mL, 54 mmol). The mixture was stirred at this temperature for 20 minutes, warmed to room temperature and stirred for another 20 minutes and then re-cooled to −78° C. The solution was treated with iodomethane (7.7 g, 54 mmol) dropwise. The mixture was then slowly allowed to warm to room temperature and stirred overnight, quenched with ammonium chloride (200 mL). The mixture was extracted with ethyl acetate (3×100 mL). The combined organic extracts were dried over anhydrous sodium sulfate and concentrated. The residue was chromatographed over 0-20% ethyl acetate in petroleum ether to give the title compound (2 g, 47%) as a yellow oil. MS (ESI): 385 m/z [M+H]⁺, retention time: 1.67 minutes; purity: >99% (254 nm) (LC-MS method 9).

Intermediate 47: Methyl 4-((2,2-difluorobut-3-yn-1-yl)oxy)-2-(3-iodophenyl)-2-methylbutanoate

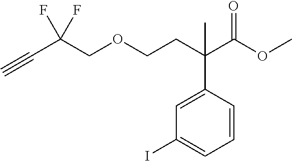 [SMILES: C#CC(F)(F)COCCC(C)(C(=O)OC)c1cccc(I)c1]

To a stirred solution of methyl 2-(3-iodophenyl)-2-methyl-4-((2-oxobut-3-yn-1-yl)oxy)butanoate (Intermediate 47F, 825 mg, 1.34 mmol) in [C8 mim][PF6] (20 mL) was added diethylaminosulfur trifluoride (0.88 mL, 6.7 mmol). The mixture was stirred at 50° C. for four hours, cooled to room temperature and diluted with ethyl acetate (50 mL). The solution was washed with water, brine, dried over sodium sulfate, and concentrated. The residue was purified by automated flash chromatography (12 g silica gel column, eluting with 0-30% ethyl acetate in petroleum ether) to give the title compound (480 mg, 68%) as colorless oil. MS (ESI): 445 m/z [M+Na]⁺, retention time: 2.14 minutes; purity: 80% (254 nm) (LC-MS method 5).

Intermediate 47A: Methyl 4-(2-acetoxyethoxy)-2-(3-iodophenyl)-2-methylbutanoate

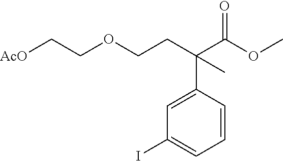 [SMILES: COC(=O)C(C)(CCOCCOC(C)=O)c1cccc(I)c1]

To a stirred solution of methyl 2-(3-iodophenyl)-2-methyl-4-(2-(tosyloxy)ethoxy)butanoate (Intermediate 7A-18, 8.2 g, 13.2 mmol) in N,N-dimethylformamide (50 mL) was added potassium acetate (3.9 g, 39.7 mmol) and 18-Crown-6 (3.5 g, 13.2 mmol). The mixture was stirred at 100° C. for 16 hours, cooled to room temperature, and diluted with ethyl acetate (100 mL). The solution was washed with water, brine, dried over sodium sulfate, and concentrated. The residue was purified by automated flash chromatography (40 g silica gel column, eluting with 0-30% ethyl acetate in petroleum ether) to give the title compound (5.2 g, 85.0%) as colorless oil. MS (ESI): 443 m/z [M+Na]⁺, retention time: 2.12 minutes; purity: >99% (254 nm) (LC-MS method 5).

Intermediate 47B: Methyl 4-(2-hydroxyethoxy)-2-(3-iodophenyl)-2-methylbutanoate

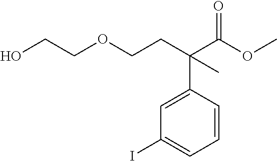 [SMILES: COC(=O)C(C)(CCOCCO)c1cccc(I)c1]

To a solution of methyl 4-(2-acetoxyethoxy)-2-(3-iodophenyl)-2-methylbutanoate (Intermediate 47A, 5.2 g, 11.3 mmol) in methanol (50 mL) was added potassium carbonate (3.11 g, 22.5 mmol). The mixture was stirred at room temperature for one hour and concentrated. The residue was partitioned between ethyl acetate (100 mL) and water (50 mL). The separated organic layer, combined with one additional ethyl acetate extract (50 mL) was dried over sodium sulfate, and concentrated. The residue was purified by automated flash chromatography (40 g silica gel column, eluting with 0-30% ethyl acetate in petroleum) to give the title compound (4.6 g, 98%) as colorless oil. MS (ESI): 401 m/z [M+Na]⁺, retention time: 1.95 minutes; purity: 91% (254 nm) (LC-MS method 5).

Intermediate 47C: Methyl 2-(3-iodophenyl)-2-methyl-4-(2-oxoethoxy)butanoate

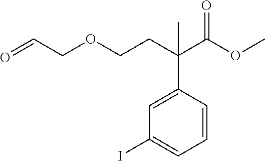 [SMILES: COC(=O)C(C)(CCOCC=O)c1cccc(I)c1]

To a stirred solution of methyl 4-(2-hydroxyethoxy)-2-(3-iodophenyl)-2-methylbutanoate (Intermediate 47B, 4.6 g, 11.1 mmol) in dichloromethane (50 mL) was added Dess-Martin Periodinane (5.63 g, 13.3 mmol). The mixture was stirred at room temperature for two hours, quenched with saturated sodium bicarbonate (100 mL). The solution was extracted with dichloromethane (2×100 mL). The combined organic phases were washed with brine, dried over sodium sulfate, and concentrated. The residue was purified by automated flash chromatography (40 g silica gel column, eluting with 0-20% ethyl acetate in petroleum) to give the title compound (2.9 g, 57%) as colorless oil. MS (ESI): 377 m/z [M+H]⁺, retention time: 1.96 minutes; purity: 82% (254 nm) (LC-MS method 5).

Intermediate 47D: Mixture of methyl 4-((2-hydroxybut-3-yn-1-yl)oxy)-2-(3-iodophenyl)-2-methylbutanoate and methyl 4-((2-hydroxy-4-(trimethylsilyl)but-3-1-yn-1-yl)oxy)-2-(3-iodophenyl)-2-methylbutanoate

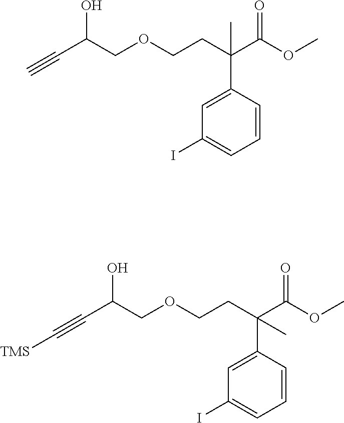 [SMILES: C#CC(O)COCCC(C)(C(=O)OC)c1cccc(I)c1 ; COC(=O)C(C)(CCOCC(O)C#C[Si](C)(C)C)c1cccc(I)c1]

To a stirred and cooled (−78° C.) solution of trimethylsilylacetylene (776 mg, 7.9 mmol) in tetrahydrofuran (25 mL) was added n-butyl lithium (3.3 mL, 8.22 mmol, 2.5 M in hexanes). The mixture was stirred at this temperature for one hour, then treated with a solution of methyl 2-(3-iodophenyl)-2-methyl-4-(2-oxoethoxy)butanoate (Intermediate 47C, 2.9 g, 6.32 mmol) in tetrahydrofuran (25 mL) dropwise and stirred for an additional one hour at the same temperature. The reaction was quenched with water and extracted with ethyl acetate (3×50 mL). The combined organic phases were washed with brine, dried over sodium sulfate, and concentrated. The residue was purified by automated flash chromatography (40 g silica gel column, eluting with 0-20% ethyl acetate in petroleum) to give the title compound (2.5 g, 58%) as colorless oil. MS (ESI): 497 m/z [M+Na]⁺, retention time: 2.22 minutes; purity: 70% (254 nm) (LC-MS method 5) (TMS protected product).

Intermediate 47E: Methyl 4-((2-hydroxybut-3-yn-1-yl)oxy)-2-(3-iodophenyl)-2-methylbutanoate

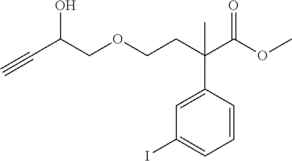 [SMILES: C#CC(O)COCCC(C)(C(=O)OC)c1cccc(I)c1]

To a stirred solution of the mixture of methyl 4-((2-hydroxybut-3-yn-1-yl)oxy)-2-(3-iodophenyl)-2-methylbutanoate and methyl 4-((2-hydroxy-4-(trimethylsilyl)but-3-yn-1-yl)oxy)-2-(3-iodophenyl)-2-methylbutanoate (Intermediate 47D, 2.5 g, 3.69 mmol) in methanol (50 mL) was added potassium carbonate (765 mg, 5.53 mmol). The reaction was stirred at room temperature for one hour, diluted with ethyl acetate (50 mL). The solution was washed with water, brine, dried over sodium sulfate, and concentrated. The residue was purified by automated flash chromatography (40 g silica gel column, eluting with 0-30% ethyl acetate in petroleum) to give the title compound (1 g, 54%) as colorless oil. MS (ESI): 425 m/z [M+Na]⁺, retention time: 1.93 minutes; purity: 80% (254 nm) (LC-MS method 5).

Intermediate 47F: Methyl 2-(3-iodophenyl)-2-methyl-4-((2-oxobut-3-yn-1-yl)oxy)butanoate

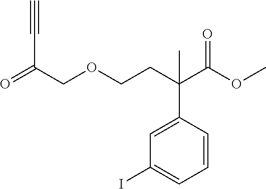 [SMILES: C#CC(=O)COCCC(C)(C(=O)OC)c1cccc(I)c1]

To a stirred solution of methyl 4-(2-hydroxybut-3-ynoxy)-2-(3-iodophenyl)-2-methylbutanoate (Intermediate 47E, 1 g, 2.49 mmol) in dichloromethane (20 mL) was added Dess-Martin Periodinane (1.27 mg, 2.98 mmol). The mixture was stirred at room temperature for one hour, quenched with saturated sodium bicarbonate (50 mL). The solution was extracted with dichloromethane (2×50 mL). The combined organic phases were washed with brine, dried over sodium sulfate, and concentrated. The residue was purified by automated flash chromatography (20 g silica gel column, eluting with 0-20% ethyl acetate in petroleum) to give the title compound (825 mg, 54%) as colorless oil. MS (ESI): 423 m/z [M+Na]⁺, retention time: 2.03 minutes; purity: 65% (254 nm) (LC-MS method 5).

Intermediate 48A: Methyl 2-(3-iodophenyl)-2,5-dimethyl-5-(prop-2-yn-1-yloxy)hexanoate

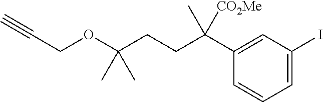 [SMILES: C#CCOC(C)(C)CCC(C)(C(=O)OC)c1cccc(I)c1]

To a stirred solution of methyl 2-(3-iodophenyl)-2,5-dimethylhex-5-enoate (Intermediate 7A-19, 5.1 g, 14.2 mmol) in dichloromethane (30 mL) was added prop-2-yn-1-ol (2.39 g, 42.7 mmol) and Amberlyst 15 (5 g). The mixture was stirred for 16 hours, then partitioned between water (50 mL) and ethyl acetate (50 mL). The separated organic phase, combined with two additional ethyl acetate extracts, was washed with brine, dried over sodium sulfate, and concentrated. The residue was purified by column chromatography on silica gel using ethyl acetate/petroleum ether (10:90) to afford the title compound as oil (4.6 g, 78%). ¹H NMR (400 MHz, CDCl₃) δ 7.64 (t, J=1.7 Hz, 1H), 7.60-7.56 (m, 1H), 7.28-7.25 (m, 1H), 7.06 (t, J=7.9 Hz, 1H), 4.03-3.91 (m, 2H), 3.67 (s, 3H), 2.41-2.33 (m, 1H), 2.18-1.88 (m, 2H), 1.54-1.52 (m, 3H), 1.39-1.29 (m, 2H), 1.20-1.18 (m, 6H) ppm.

Intermediate 49: 2-(3-Iodophenyl)-2,7-dimethyl-7-(2-oxooxazolidin-5-yl)octanoic acid

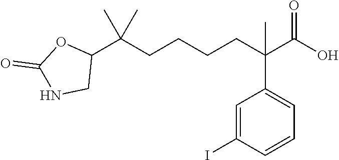 [SMILES: CC(CCCCC(C)(C)C1CNC(=O)O1)(C(=O)O)c1cccc(I)c1]

To a solution of methyl 9-(((benzyloxy)carbonyl)amino)-8-hydroxy-2-(3-iodophenyl)-2,7,7-trimethylnonanoate (Intermediate 49B, 11 g, 18.9 mmol) in tetrahydrofuran (100 mL), methanol (50 mL) and water (10 mL) was added lithium hydroxide (15.9 g, 378 mmol). The resulting mixture was stirred at room temperature for 3 days, and concentrated. The residue was acidified by 1 N hydrochloric acid to pH ˜3 and extracted with ethyl acetate (3×200 mL). The combined organic layers were dried over sodium sulfate and concentrated. The residue was purified by automated flash chromatography eluting with methanol/dichloromethane=1/10 to afford the title compound (5.3 g, 61%) as colorless oil. MS (ESI): 460 m/z [M+H]⁺, retention time: 1.99 minutes; purity: 98% (254 nm) (LC-MS method 6).

Intermediate 49A: Methyl 2-(3-iodophenyl)-2,7,7-trimethylnon-8-enoate

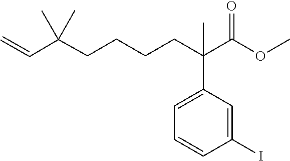 [SMILES: C=CC(C)(C)CCCCC(C)(C(=O)OC)c1cccc(I)c1]

To a stirred and cooled (0° C.) solution of methyltriphenylphosphonium bromide (52.4 g, 130 mmol) in tetrahydrofuran (200 mL) was added sodium hydride (3.46 g, 86.5 mmol). The mixture was stirred at 0° C. for 30 minutes, then treated with methyl 2-(3-iodophenyl)-2,7,7-trimethyl-8-oxooctanoate (Intermediate 28B, 18 g, 43.2 mmol). The reaction was stirred at room temperature for 2 hours, quenched with water. The mixture was extracted with ethyl acetate (3×200 mL). The combined organic layers were dried over sodium sulfate and concentrated. The residue was purified with automated flash chromatography (eluting with ethyl acetate/petroleum ether=1/20 to afford the title compound (12.5 g, 70%) as a colorless oil. MS (ESI): 437 m/z [M+Na]⁺, retention time: 2.61 minutes; purity: 85% (254 nm) (LC-MS method 6).

The following intermediate was prepared based on the procedures described for Intermediate 49A

| Inter. | | | MS m/z |
|---|---|---|---|
| No. | Structure | Name | [M + H]⁺ |
| 49A-1 | 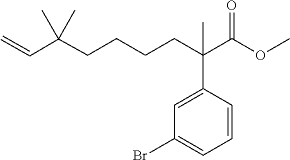 [SMILES: C=CC(C)(C)CCCCC(C)(C(=O)OC)c1cccc(Br)c1] | methyl 2-(3-bromophenyl)-2,7,7-trimethylnon-8-enoate | 367, 369 |

Intermediate 49B: Methyl 9-(((benzyloxy)carbonyl)amino)-8-hydroxy-2-(3-iodophenyl)-2,7,7-trimethylnonanoate

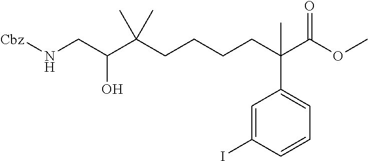 [SMILES: COC(=O)C(C)(CCCCC(C)(C)C(O)CNC(=O)OCc1ccccc1)c1cccc(I)c1]

To a stirred solution of benzyl ((4-chlorobenzoyl)oxy)carbamate (12.9 g, 42.1 mmol) in acetonitrile (150 mL) was added osmium tetroxide (308 mg, 1.2 mmol). The mixture and stirred at room temperature for 10 minutes, then treated with a solution of methyl 2-(3-iodophenyl)-2,7,7-trimethylnon-8-enoate (12.4 g, 30 mmol) in acetonitrile (150 mL), followed by water (30 mL). The reaction was stirred at room temperature for 18 hours, quenched with potassium dithionite solution and stirred for a further 5 minutes. The solution was partitioned between water (100 mL) and ethyl acetate (200 mL). The separated organic layer, combined with two additional ethyl acetate extracts (2×100 mL), was dried over sodium sulfate, and concentrated. The residue was purified by automated flash chromatography eluting with ethyl acetate/petroleum ether=1/2 to afford the title compound (11 g, 63%) as colorless oil. MS (ESI): 582 m/z [M+H]⁺, retention time: 2.43 minutes; purity: 90% (254 nm) (LC-MS method 6).

The following intermediate was prepared based on the procedures described for Intermediate 49B

| Inter. | | | MS m/z |
|---|---|---|---|
| No. | Structure | Name | [M + H]⁺ |
| 49B-1 | 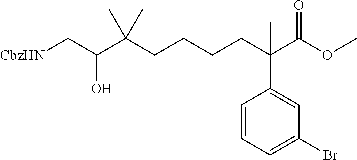 [SMILES: COC(=O)C(C)(CCCCC(C)(C)C(O)CNC(=O)OCc1ccccc1)c1cccc(Br)c1] | methyl 9-(((benzyloxy)carbonyl)amino)-2-(3-bromophenyl)- 8-hydroxy-2,7,7-trimethylnonanoate | 534, 536 |

Intermediate 50: 2-(3-Iodophenyl)-2,5-dimethyl-7-(2-oxooxazolidin-4-yl)heptanoic acid

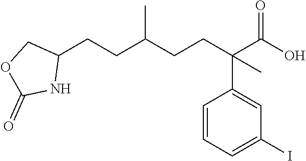 [SMILES: CC(CCC1COC(=O)N1)CCC(C)(C(=O)O)c1cccc(I)c1]

To a stirred solution of 8-((tert-butoxycarbonyl)amino)-9-hydroxy-2-(3-iodophenyl)-2,5-dimethylnonanoic acid (Intermediate 50G, 2.0 g, 0.00385 mol) in tetrahydrofuran (30 mL) was added sodium hydride (0.77 g, 0.0193 mol). The reaction was stirred at room temperature for 2 hours, quenched with 0.5 M hydrochloric acid (100 mL). The solution was extracted with ethyl acetate (3×50 mL). The combined organic layers were dried over sodium sulfate, and concentrated. The residue was purified by silica gel column chromatography (dichloromethane/methanol=15/1) to give the title compound (1.7 g, 99%) as a yellow oil. MS (ESI): 446 m/z [M+H]⁺, retention time: 1.97 minutes; purity: 74% (214 nm) (LC-MS method 6).

Intermediate 50A: Methyl 7-acetoxy-2-(3-iodophenyl)-2,5-dimethylheptanoate

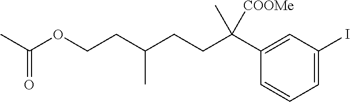 [SMILES: COC(=O)C(C)(CCC(C)CCOC(C)=O)c1cccc(I)c1]

To a stirred solution of methyl 7-bromo-2-(3-iodophenyl)-2,5-dimethylheptanoate (Intermediate 7A-4, 7.8 g, 0.0172 mol) in dry dimethylformamide (50.0 mL) was added anhydrous potassium acetate (5.06 g, 0.0516 mol), 18-crown-6 (4.55 g, 0.0172 mol). The reaction was stirred at 80° C. for 16 hours, cooled to room temperature. The mixture was partitioned between water (80 mL) and ethyl acetate(80 mL). The separated organic layer, combined with two additional ethyl acetate extracts, was washed with brine, dried over magnesium sulfate, and concentrated. The crude title compound (7.4 g, 99%) was used for the next step without further purification. MS (ESI): 455 m/z [M+Na]⁺, retention time: 2.38 minutes; purity: 75% (214 nm) (LC-MS method 6).

Intermediate 50B: Methyl 7-hydroxy-2-(3-iodophenyl)-2,5-dimethylheptanoate

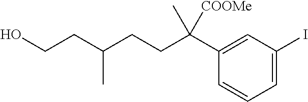 [SMILES: COC(=O)C(C)(CCC(C)CCO)c1cccc(I)c1]

To a stirred solution of methyl 7-acetoxy-2-(3-iodophenyl)-2,5-dimethylheptanoate (Intermediate 50A, 7.4 g, 0.0171 mol) in methanol (100.0 mL) was added potassium carbonate (4.73 g, 0.0342 mol). The reaction was stirred for 16 hours and concentrated. The residue was partitioned between water (50 mL) and ethyl acetate (100 mL). The separated organic layer, combined with two additional ethyl acetate extracts (2×50 mL), was washed with brine, dried over magnesium sulfate, and concentrated. The residue was purified by silica gel column chromatography (eluting with petroleum ether/ethyl acetate=2/1) to give the title compound (6.6 g, 99%) as a colorless oil. MS (ESI): 413 m/z [M+Na]⁺, retention time: 2.17 minutes; purity: 94% (214 nm) (LC-MS method 6).

Intermediate 50C: Methyl 2-(3-iodophenyl)-2,5-dimethyl-7-oxoheptanoate

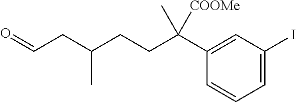 [SMILES: COC(=O)C(C)(CCC(C)CC=O)c1cccc(I)c1]

To a stirred solution of methyl 7-hydroxy-2-(3-iodophenyl)-2,5-dimethylheptanoate (Intermediate 50B, 6.6 g, 0.0169 mol) in dichloromethane (100 mL) was added silica gel (10 g), and pyridinium chlorochromate (5.47 g, 0.0254 mol). The reaction was stirred for 3 hours, then diluted with dichloromethane (50 mL). The solution was passed through a short column of silica gel column to afford the crude title compound (6.4 g, 98%) as a yellow oil, which was used for next step without further purification. MS (ESI): 389 m/z [M+H]⁺, retention time: 2.26 minutes; purity: 78% (214 nm) (LC-MS method 6).

Intermediate SOD: Dimethyl (Z)-2-((tert-butoxycarbonyl)amino)-8-(3-iodophenyl)-5,8-dimethylnon-2-enedioate

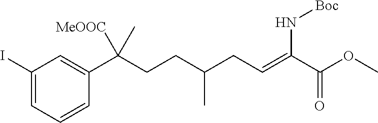 [SMILES: COC(=O)/C(=C/CC(C)CCC(C)(C(=O)OC)c1cccc(I)c1)NC(=O)OC(C)(C)C]

To a stirred solution of methyl 2-(3-iodophenyl)-2,5-dimethyl-7-oxoheptanoate (Intermediate 50C, 6.4 g, 0.0165 mol) in dichloromethane (50 mL) was added 1,8-diazabicyclo[5.4.0]undec-7-ene (3.01 g, 0.0198 mol) and methyl 2-(tert-butoxycarbonylamino)-2-dimethoxyphosphoryl-acetate (5.39 g, 0.0181 mol). The mixture was stirred for 16 hours and concentrated. The residue was purified by silica gel column chromatography (eluting with petroleum ether/ethyl acetate=4:1) to the title compound (7.7 g, 0.0138 mol) as a colorless oil. MS (ESI): 582 m/z [M+Na]⁺, retention time: 2.28 minutes; purity: 95% (214 nm) (LC-MS method 6).

Intermediate 50E: Dimethyl 8-((tert-butoxycarbonyl)amino)-2-(3-iodophenyl)-2,5-dimethylnonanedioate

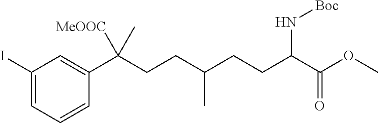 [SMILES: COC(=O)C(CCC(C)CCC(C)(C(=O)OC)c1cccc(I)c1)NC(=O)OC(C)(C)C]

To a stirred solution of dimethyl (Z)-2-((tert-butoxycarbonyl)amino)-8-(3-iodophenyl)-5,8-dimethylnon-2-enedioate (Intermediate 50D, 7.7 g, 0.0138 mol) in dichloromethane (40 mL) was added trifluoracetic acid (10 mL) and triethylsilane (4.8 g, 0.0413 mol). The mixture was stirred for 16 hours and concentrated. The residue was neutralized with saturated sodium bicarbonate solution (50 mL) and diluted with tetrahydrofuran (50 mL). To this mixture was added di-tert-butyl dicarbonate (4.51 g, 0.0206 mol). After stirring for another 16 hours, the mixture was extracted with ethyl acetate (3×50 mL). The combined organic extracts were washed with brine, dried over sodium sulfate, and concentrated. The residue was purified by silica gel column chromatography (petroleum ether/ethyl acetate=4:1) to afford the title compound (7.0 g, 91%) as a yellow oil. MS (ESI): 584 m/z [M+Na]⁺, retention time: 2.32 minutes; purity: 95% (214 nm) (LC-MS method 6).

Intermediate 50F: Methyl 8-((tert-butoxycarbonyl)amino)-9-hydroxy-2-(3-iodophenyl)-2,5-dimethylnonanoate

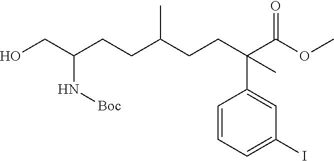 [SMILES: COC(=O)C(C)(CCC(C)CCC(CO)NC(=O)OC(C)(C)C)c1cccc(I)c1]

To a stirred and cooled (−78° C.) solution of dimethyl 8-((tert-butoxycarbonyl)amino)-2-(3-iodophenyl)-2,5-dimethylnonanedioate (Intermediate 50E, 7.0 g, 0.0125 mol) in tetrahydrofuran (100 mL) was added lithium aluminum hydride (0.984 g, 0.0249 mol). The reaction was allowed to warm to room temperature and stirred for 16 hours, then quenched carefully with sodium sulfate decahydrate (5 g) and stirred for 15 minutes. The mixture was filtered. The filtrate was concentrated. The residue was purified by silica gel column chromatography (petroleum ether/ethyl acetate=1:1) to give the title compound (2.6 g, 39%) as a yellow oil. MS (ESI): 556 m/z [M+Na]⁺, retention time: 2.02 minutes; purity: 77% (214 nm) (LC-MS method 6).

Intermediate 50G: 8-((Tert-Butoxycarbonyl)amino)-9-hydroxy-2-(3-iodophenyl)-2,5-dimethylnonanoic acid

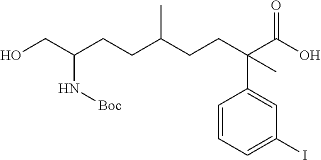 [SMILES: CC(CCC(CO)NC(=O)OC(C)(C)C)CCC(C)(C(=O)O)c1cccc(I)c1]

To a stirred solution of methyl 8-((tert-butoxycarbonyl)amino)-9-hydroxy-2-(3-iodophenyl)-2,5-dimethylnonanoate (Intermediate 50F, 2.6 g, 0.00487 mol) in tetrahydrofuran/water/methanol (20 mL/10 mL/10 mL) was added lithium hydroxide monohydrate (2.05 g, 0.0487 mol). The reaction was stirred for 16 hours and concentrated. The residue was neutralized with 1 M hydrochloric acid to pH 2-3 and extracted with ethyl acetate (4×50 mL). The combined organic layers were dried over sodium sulfate and concentrated to give the title compound (2.2 g, 87%) as a yellow oil. MS (ESI): 542 m/z [M+Na]⁺, retention time: 2.11 minutes; purity: 64% (214 nm) (LC-MS method 6).

Intermediate 51: 3-((2-Methylbut-3-en-2-yl)oxy)propyl 4-methylbenzenesulfonate

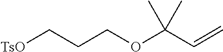 [SMILES: C=CC(C)(C)OCCCOS(=O)(=O)c1ccc(C)cc1]

To a stirred and cooled (0° C.) solution of 3-((2-methylbut-3-en-2-yl)oxy)propan-1-ol (Intermediate 51B, 10.5 g, 0.073 mol) in dichloromethane (100 mL) was added N,N-dimethylpyridin-4-amine (0.89 g, 0.0073 mol), triethylamine (22 mL, 0.22 mol) and a solution of tosyl chloride (16.7 g, 0.087 mol) in dichloromethane (80 mL). The reaction was stirred at room temperature overnight. The mixture was washed with water (2×50 mL), dried over sodium sulfate, and concentrated. The residue was purified by flash chromatography (330 g silica gel column, eluting with 0-25% ethyl acetate in petroleum ether) to give the title compound (20 g, 92%) as an oil. 1H NMR (400 MHz, CDCl₃) δ 7.79 (d, J=7.4 Hz, 2H), 7.35 (d, J=7.9 Hz, 2H), 5.70 (dd, J=17.8, 10.6 Hz, 1H), 5.14-5.01 (m, 2H), 4.13 (t, J=6.2 Hz, 2H), 3.30 (t, J=5.9 Hz, 2H), 2.45 (s, 3H), 1.91-1.79 (m, 2H), 1.18 (s, 6H) ppm.

The following intermediate was prepared based on the procedures described for Intermediate 51

| Inter. | | | MS m/z |
|---|---|---|---|
| No. | Structure | Name | [M + H]⁺ |
| 51-1 | 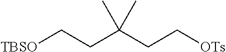 [SMILES: Cc1ccc(S(=O)(=O)OCCC(C)(C)CCO[Si](C)(C)C(C)(C)C)cc1] | 5-((tert-butyldimethylsilyl)oxy)-3,3-dimethylpentyl 4- methylbenzenesulfonate | 401 |

Intermediate 51A: ethyl 3-((2-methylbut-3-en-2-yl)oxy)propanoate

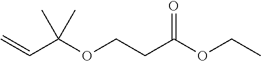

To a stirred and cooled (0° C.) solution of ethyl acrylate (25 g, 0.25 mol) and 2 2-methylbut-3-en-2-ol (43 g, 0.5 mmol) in tetrahydrofuran (200 mL) was added sodium hydride (60% in mineral oil, 1 g, 0.025 mol). The reaction was stirred at 25° C. overnight, quenched with water. The mixture was extracted with ethyl acetate (3×150 mL). The combined organic extracts were washed with brine (60 mL), dried over sodium sulfate, and concentrated. The residue was purified by automated flash chromatography (330 g silica gel column, eluting with 0-10% ethyl acetate in petroleum ether) to give the title compound (13.7 g, 29%) as an oil. ¹H NMR (400 MHz, CDCl₃) δ 5.82 (dd, J=17.6, 10.8 Hz, 1H), 5.16-5.09 (m, 2H), 4.15 (q, J=7.1 Hz, 2H), 3.57 (t, J=6.6 Hz, 2H), 2.51 (t, J=6.6 Hz, 2H), 1.28-1.22 (m, 9H) ppm.

Intermediate 51B: 3-((2-Methylbut-3-en-2-yl)oxy)propan-1-ol

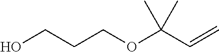

To a stirred and cooled (0° C.) solution of ethyl 3-((2-methylbut-3-en-2-yl)oxy)propanoate (Intermediate 51A, 21.8 g, 0.117 mol) in tetrahydrofuran (200 mL) was added lithium aluminum hydride (1 M in tetrahydrofuran, 140 mL, 0.014 mol). The reaction was stirred at room temperature for 1 hour, then cooled to room temperature and carefully quenched with water (6 mL), followed by 6 mL of 15% sodium hydroxide, and 18 mL of water. The mixture was stirred at room temperature for 15 minutes and filtered. The filter cake was washed by tetrahydrofuran (80 mL). The filtrate was dried over sodium sulfate and concentrated. The residue was purified by flash chromatography (330 g silica gel column, eluting with 0-50% ethyl acetate in petroleum ether) to give the title compound (10.5 g, 62%) as an oil. ¹H NMR (400 MHz, CDCl₃) δ 5.82 (dd, J=17.6, 10.9 Hz, 1H), 5.16-5.11 (m, 2H), 3.75 (t, J=5.6 Hz, 2H), 3.50 (t, J=5.8 Hz, 2H), 3.01 (brs, 1H), 1.79-1.72 (m, 2H), 1.26 (s, 6H) ppm.

Intermediate 52: 1-(((Benzyloxy)carbonyl)amino)-3-((4-(3-iodophenyl)-4-methyl-5-oxohexyl)oxy)-3-methylbutan-2-yl acetate

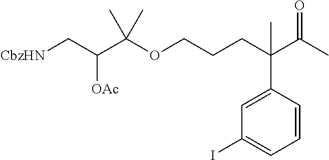 [SMILES: CC(=O)OC(CNC(=O)OCc1ccccc1)C(C)(C)OCCCC(C)(C(C)=O)c1cccc(I)c1]

To a stirred solution of benzyl (2-hydroxy-3-((4-(3-iodophenyl)-4-methyl-5-oxohexyl)oxy)-3-methylbutyl)carbamate (Intermediate 52D, 7.91 g, 13.9 mmol) in dichloromethane (100 mL) was added acetic anhydride (1.98 mL, 20.9 mmol), N,N-dimethylpyridin-4-amine (170 mg, 1.39 mol) and triethylamine (7.77 mL, 55.8 mol). The reaction was stirred at room temperature for 2 hours. The mixture was washed with water, brine, dried over sodium sulfate, and concentrated. The residue was purified by automated flash chromatography (120 g silica gel column, eluting with 0-40% ethyl acetate in petroleum ether) to give the title compound (7.8 g, 91%) as an oil. MS (ESI): 632 m/z [M+Na]⁺, retention time: 2.41 minutes; purity: 95% (214 nm) (LC-MS method 2).

Intermediate 52A: 2-(3-Iodophenyl)-2-methyl-5-((2-methylbut-3-en-2-yl)oxy)pentanal

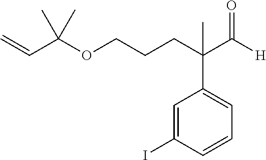 [SMILES: C=CC(C)(C)OCCCC(C)(C=O)c1cccc(I)c1]

To a stirred and cooled (−78° C.) solution of 2-(3-iodophenyl)-N-methoxy-N,2-dimethyl-5-((2-methylbut-3-en-2-yl)oxy)pentanamide (Intermediate 33A-1, 9.2 g, 20.7 mmol) in tetrahydrofuran (150 mL) was added diisobutylaluminum hydride (1 M in hexane, 62 mL, 62 mmol). The reaction was stirred at −78° C. for 1 hour, then warmed to room temperature and stirred for an additional 3 hours. The mixture was quenched with saturated potassium sodium tartrate (150 mL), stirred until the solution turned clear, and extracted with ethyl acetate (3×150 mL). The combined organic extracts were washed with 1 M hydrochloric acid, and brine, dried over sodium sulfate, and concentrated to give the title compound (7.4 g, 92%) as an oil. MS (ESI): 409 m/z [M+Na]⁺, retention time: 2.55 minutes; purity: 49% (214 nm) (LC-MS method 2).

The following intermediate was prepared based on the procedures described for Intermediate 52A

| Inter. | | | MS m/z |
|---|---|---|---|
| No. | Structure | Name | [M + H]⁺ |
| 52A-1 | 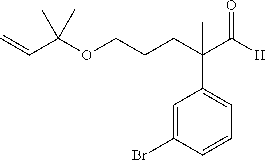 [SMILES: C=CC(C)(C)OCCCC(C)(C=O)c1cccc(Br)c1] | 2-(3-bromophenyl)-2-methyl-5-((2-methylbut-3-en-2- yl)oxy)pentanal | 361, 363 [M + Na]⁺ |

Intermediate 52B: 3-(3-Iodophenyl)-3-methyl-6-((2-methylbut-3-en-2-yl)oxy)hexan-2-ol

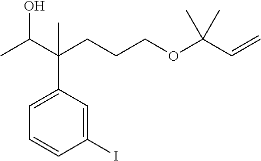 [SMILES: C=CC(C)(C)OCCCC(C)(c1cccc(I)c1)C(C)O]

To a stirred and cooled (0° C.) solution of 2-(3-iodophenyl)-2-methyl-5-((2-methylbut-3-en-2-yl)oxy)pentanal (Intermediate 52A, 7.4 g,19.2 mmol) in tetrahydrofuran (100 mL) was added methyl magnesium bromide (3 M in 2-methyl-tetrahydrofuran) (9.58 mL, 28.7 mmol). The mixture was stirred at 0° C. for one hour, quenched with saturated ammonium chloride (30 mL), and extracted with ethyl acetate (3×100 mL). The combined organic extracts were washed with brine, dried over sodium sulfate, and concentrated. The residue was purified by flash chromatography (80 g silica gel column, eluting with 0-40% ethyl acetate in petroleum ether) to give the title compound (6 g, 77%) as an oil. MS (ESI): 425 m/z [M+Na]⁺, retention time: 2.49 minutes; purity: 78% (214 nm) (LC-MS method 2).

Intermediate 52C: 3-(3-Iodophenyl)-3-methyl-6-((2-methylbut-3-en-2-yl)oxy)hexan-2-one

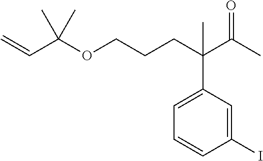 [SMILES: C=CC(C)(C)OCCCC(C)(C(C)=O)c1cccc(I)c1]

To a stirred solution of 3-(3-iodophenyl)-3-methyl-6-((2-methylbut-3-en-2-yl)oxy)hexan-2-ol (Intermediate 52B, 6 g, 14.9 mmol) in dimethyl sulfoxide (150 mL) was added stabilized 2-iodoxybenzoic acid (61717-82-6, 46%) (12.7 g, 20.9 mol). The reaction was stirred at 40° C. for two hours, then diluted with ethyl acetate (400 mL). The solution was washed with 1 M sodium hydroxide, brine, dried over sodium sulfate, and concentrated. The residue was purified by automated flash chromatography (40 g silica gel column, eluting with 0-40% ethyl acetate in petroleum) to give the title compound (5 g, 84%) as an oil. MS (ESI): 423 m/z [M+Na]⁺, retention time: 2.56 minutes; purity: 93% (214 nm) (LC-MS method 2).

The following intermediate was prepared based on the procedures described for Intermediate 52C.

| Inter. | | | MS m/z |
|---|---|---|---|
| No. | Structure | Name | [M + H]⁺ |
| 52C-1 | 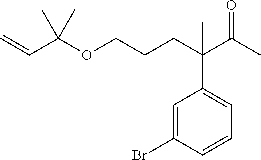 [SMILES: C=CC(C)(C)OCCCC(C)(C(C)=O)c1cccc(Br)c1] | 3-(3-bromophenyl)-3-methyl-6-((2-methylbut-3-en-2- yl)oxy)hexan-2-one | 375, 377 [M + Na]⁺ |

Intermediate 52D: Benzyl (2-hydroxy-3-((4-(3-iodophenyl)-4-methyl-5-oxohexyl)oxy)-3-methylbutyl)carbamate

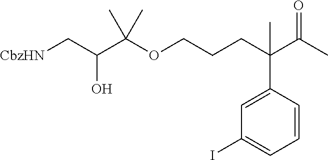 [SMILES: CC(=O)C(C)(CCCOC(C)(C)C(O)CNC(=O)OCc1ccccc1)c1cccc(I)c1]

To a stirred solution of benzyl ((4-chlorobenzoyl)oxy)carbamate (5.73 g, 18.7 mmol) in acetonitrile (80 mL) was added osmium tetroxide (127 mg, 0.5 mmol). The reaction was stirred at room temperature for 10 minutes, then treated with a solution of 3-(3-iodophenyl)-3-methyl-6-((2-methylbut-3-en-2-yl)oxy)hexan-2-one (Intermediate 52C, 5 g, 12.5 mmol) in acetonitrile (80 mL), followed by the addition of water (20 mL). The mixture was stirred at room temperature for 16 hours, quenched with saturated sodium thiosulfate (55 mL), and stirred for a further 5 minutes. The solution was partitioned between water (50 mL) and ethyl acetate (100 mL). The separated organic layer, combined with two additional ethyl acetate extracts (2×50 mL), was washed with saturated sodium bicarbonate (60 mL), brine, dried over magnesium sulfate, and concentrated. The residue was purified by automated flash chromatography (120 g silica gel column; eluting with ethyl acetate/petroleum ether from 0% to 50%) to give the title compound (6.01 g, 85%) as an oil. MS (ESI): 590 m/z [M+Na]⁺, retention time: 2.34 minutes; purity: 82% (214 nm) (LC-MS method 2).

Intermediate 53: 5-((4,6-Difluoro-1H-benzo[d]imidazol-5-yl)oxy)-2-fluorobenzonitrile

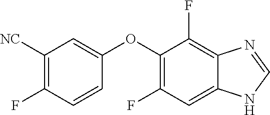 [SMILES: N#Cc1cc(Oc2c(F)cc3[nH]cnc3c2F)ccc1F]

To a solution of 5-(3-amino-2,6-difluoro-4-nitrophenoxy)-2-fluorobenzonitrile (Intermediate 53B, 2.4 g, 7.8 mmol), ammonium chloride (4.2 g, 7.8 mmol) in formic acid (20 mL) and isopropanol (20 mL) was added iron powder (4.30 g, 77.6 mmol). The mixture was stirred at room temperature for four hours, diluted with water (100 mL) and extracted with ethyl acetate (100 mL×2). The combined extracts were washed with water (100 mL) and brine (80 mL). The solution was then dried over sodium sulfate and concentrated. The residue was purified by column chromatography on silica gel to give the title compound as yellow solid (1.8 g, 80%). MS: 290 m/z [M+H]⁺.

Intermediate 53A: 2-Fluoro-5-(2,3,6-trifluoro-4-nitrophenoxy)benzonitrile

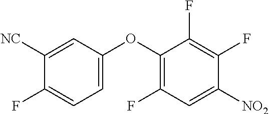 [SMILES: N#Cc1cc(Oc2c(F)cc([N+](=O)[O-])c(F)c2F)ccc1F]

To a solution of 1,2,3,4-tetrafluoro-5-nitrobenzene (2 g, 10.2 mmol) and 2-fluoro-5-hydroxybenzonitrile (1.4 g, 10.2 mmol) in DMF (20 mL) was added potassium carbonate (3 g, 21.6 mmol) and stirred at room temperature for one hour. The mixture was diluted with water (40 mL) and extracted with ethyl acetate (40 mL×2). The combined extracts were washed with water (40 mL) and brine (20 mL), filtered and concentrated. The residue was purified by column chromatography on silica gel to give the title compound as a white solid (2.2 g, 70%). ¹H NMR (400 MHz, CDCl₃) δ 7.83-7.92 (m, 1H), 7.26-7.33 (m, 2H), 7.24 (d, J=3.6 Hz, 1H) ppm. MS: 313 m/z [M+H]⁺.

Intermediate 53B: 5-(3-Amino-2,6-difluoro-4-nitrophenoxy)-2-fluorobenzonitrile

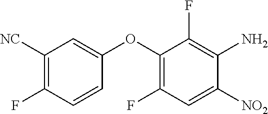 [SMILES: N#Cc1cc(Oc2c(F)cc([N+](=O)[O-])c(N)c2F)ccc1F]

To a stirred solution of 2-fluoro-5-(2,3,6-trifluoro-4-nitrophenoxy)benzonitrile (3.00 g, 9.61 mmol) and triethylamine (4.1 mL, 29 mmol) in DMF (30 mL) was added ammonium carbonate (1.10 g, 11.4 mmol). After four hours at room temperature, the mixture was diluted with water (1×100 mL) and extracted with ethyl acetate (2×90 mL). The combined extracts were washed with water (1×100 mL) and brine (1×80 mL), dried over sodium sulfate and concentrated. Crude product was obtained as a yellow solid (2.40 g, 81%). MS: 310 m/z [M+H]⁺.

Intermediate 54: 5-((4-Bromo-6-fluoro-1-(tetrahydro-2H-pyran-2-yl)-1H-benzo[d]imidazol-5-yl)oxy)-2-fluorobenzonitrile

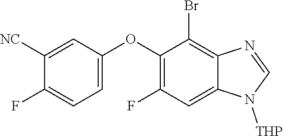

To a stirred solution of 5-((4-bromo-6-fluoro-2,3-dihydro-1H-benzo[d]imidazol-5-yl)oxy)-2-fluorobenzonitrile (Intermediate 54C 20 g, 55.2 mmol) in tetrahydrofuran (280 mL) was added 3,4-dihydro-2H-pyran (18.6 g, 221 mmol) followed by p-toluenesulfonic acid (476 mg, 2.76 mmol). The mixture was refluxed overnight, then concentrated. The residue was recrystallized in ethyl acetate/petroleum ether to afford the title compound (21 g, yield 88%) as a light-yellow solid. MS (ESI): 434, 436 m/z [M+H]⁺, retention time: 1.77 minutes, purity: >99% (214 nm) (LC-MS method 011).

Intermediate 54A: 2-bromo-3,4-difluoro-6-nitroaniline

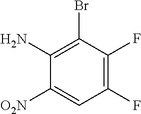 [SMILES: Nc1c([N+](=O)[O-])cc(F)c(F)c1Br]

To a stirred solution of 4,5-difluoro-2-nitro-aniline (17.4 g, 0.1 mol) in acetic acid (100 mL) was added bromine (32 g, 0.2 mol) dropwise. The mixture was stirred at 55° C. for 3 hours, then cooled to room temperature, diluted with water (500 mL), and filtered. The collected solid was dried to give the title compound (22 g, 87%) as a yellow solid. ¹H NMR (500 MHz, DMSO-d) δ 8.26 (dd, J=11.0, 8.5 Hz, 1H), 7.42 (br, 2H) ppm.

Intermediate 54B: 5-(3-Amino-2-bromo-6-fluoro-4-nitrophenoxy)-2-fluorobenzonitrile

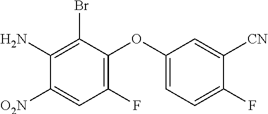 [SMILES: N#Cc1cc(Oc2c(F)cc([N+](=O)[O-])c(N)c2Br)ccc1F]

To a stirred solution of 2-bromo-3,4-difluoro-6-nitroaniline (Intermediate 85A, 25.3 g, 0.1 mol) in N,N-dimethylformamide (150 mL) was added 2-fluoro-5-hydroxy-benzonitrile (14.4 g, 0.105 mol) and potassium carbonate (27.6 g, 0.2 mol). The mixture was stirred at room temperature overnight, quenched with water (500 mL), and extracted with ethyl acetate (3×300 mL). The combined organic layers were washed with brine (3×100 mL), dried over sodium sulfate, and concentrated. The residue was crystallized with the mixture of ethyl acetate/petroleum ether (1/1) and the collected solid was dried to give the title compound (33.5 g, 88%) as a yellow solid. MS (ESI): 370, 372 m/z [M+H]⁺, retention time: 1.77 minutes, purity: 97% (214 nm) (LC-MS method 011).

Intermediate 54C: 5-((4-bromo-6-fluoro-2,3-dihydro-1H-benzo[d]imidazol-5-yl)oxy)-2-fluorobenzonitrile

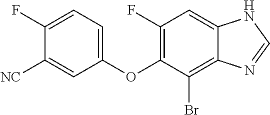 [SMILES: N#Cc1cc(Oc2c(F)cc3[nH]cnc3c2Br)ccc1F]

To a stirred solution of 5-(3-amino-2-bromo-6-fluoro-4-nitrophenoxy)-2-fluorobenzonitrile (Intermediate 85B, 15 g, 40.5 mmol) in iso-propyl alcohol (120 mL) was added iron (22.6 g, 405 mmol) followed by ammonium chloride (21.7 g, 405 mmol) and formic acid (60 mL). The mixture was stirred at 85° C. for 4 hours and concentrated. The residue partitioned between 100 mL of water and ethyl acetate (100 mL). The organic layer was combined with two additional ethyl acetate extracts (2×100 mL) and was washed with brine, dried over sodium sulfate, and concentrated. The residue was crystallized from the mixture of ethyl acetate/petroleum ether (1/1) to give the title compound (11.3 g, 77% yield) as a yellow solid. MS (ESI): 350, 352 m/z [M+H]⁺, retention time: 1.54 minutes. (LC-MS method 011)

Intermediate 55: 2-Fluoro-5-((6-fluoro-4-(hydroxymethyl)-1-(tetrahydro-2H-pyran-2-yl)-1H-benzo[d]imidazol-5-yl)oxy)benzonitrile

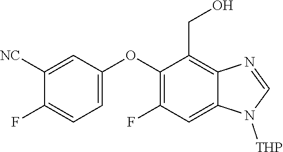

To a stirred solution of 2-fluoro-5-((6-fluoro-4-formyl-1-(tetrahydro-2H-pyran-2-yl)-1H-benzo[d]imidazol-5-yl)oxy)benzonitrile (Intermediate 55B, 4.4 g, 11.5 mmol) in tetrahydrofuran/ethanol (100 mL/10 mL) was added sodium borohydride (651 mg, 17.2 mmol). The mixture was stirred at room temperature for two hours, quenched with water (100 mL), and extracted with ethyl acetate (3×50 mL). The combined organic extracts were washed with brine (2×30 mL), dried over sodium sulfate, and concentrated. The residue was purified by flash chromatography (80 g silica gel column, eluting with 0-10% methanol in dichloromethane) to give the title compound (3.5 g, 79%) as a solid. MS (ESI): 386 m/z [M+H]⁺, retention time: 1.93 minutes, purity: >99% (254 nm) (LC-MS method 2). ¹H NMR (400 MHz, CDCl₃) δ 8.09 (s, 1H), 7.35 (d, J=9.8 Hz, 1H), 7.23-7.17 (m, 1H), 7.17-7.10 (m, 1H), 7.05-7.01 (m, 1H), 5.50-5.45 (m, 1H), 5.05 (d, J=6.3 Hz, 2H), 4.20.4.13 (m, 2H), 3.84-3.67 (m, 1H), 2.27-1.66 (m, 6H) ppm.

The following intermediates were prepared based on the procedures described for Intermediate 55 and/or for Intermediates 55A and 55B.

| Inter. | | | MS m/z |
|---|---|---|---|
| No. | Structure | Name | [M + H]⁺ |
| 55-1 | 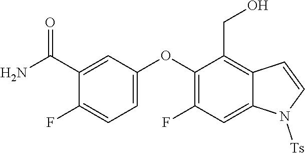 [SMILES: Cc1ccc(S(=O)(=O)n2ccc3c(CO)c(Oc4ccc(F)c(C(N)=O)c4)c(F)cc32)cc1] | 2-fluoro-5-((6-fluoro-4-(hydroxymethyl)-1-tosyl-1H-indol- 5-yl)oxy)benzamide | 455 [M − H2O + H^{]+} |
| 55-2 | 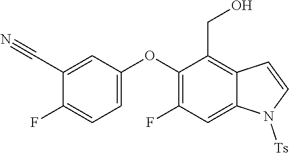 [SMILES: Cc1ccc(S(=O)(=O)n2ccc3c(CO)c(Oc4ccc(F)c(C#N)c4)c(F)cc32)cc1] | 2-fluoro-5-((6-fluoro-4-(hydroxymethyl)-1-tosyl-1H-indol- 5-yl)oxy)benzonitrile | 455 |
| 55-3 | 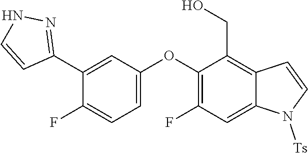 [SMILES: Cc1ccc(S(=O)(=O)n2ccc3c(CO)c(Oc4ccc(F)c(-c5cc[nH]n5)c4)c(F)cc32)cc1] | (6-fluoro-5-(4-fluoro-3-(1H-pyrazol-3-yl)phenoxy)-1-tosyl- 1H-indol-4-yl)methanol | 496 |
| 55-4 | 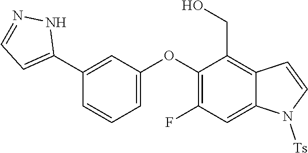 [SMILES: Cc1ccc(S(=O)(=O)n2ccc3c(CO)c(Oc4cccc(-c5ccn[nH]5)c4)c(F)cc32)cc1] | (5-(3-(1H-pyrazol-5-yl)phenoxy)-6-fluoro-1-tosyl-1H- indol-4-yl)methanol | 460 [M − H2O + H^{]+} |
| 55-5 | 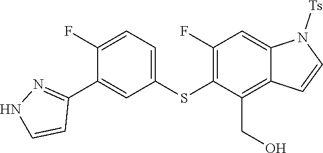 [SMILES: Cc1ccc(S(=O)(=O)n2ccc3c(CO)c(Sc4ccc(F)c(-c5cc[nH]n5)c4)c(F)cc32)cc1] | (6-fluoro-5-((4-fluoro-3-(1H-pyrazol-3-yl)phenyl)thio)-1- tosyl-1H-indol-4-yl)methanol | 512 |
| 55-6 | 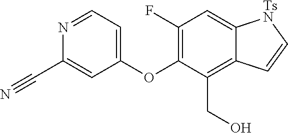 [SMILES: Cc1ccc(S(=O)(=O)n2ccc3c(CO)c(Oc4ccnc(C#N)c4)c(F)cc32)cc1] | methyl 4-((6-fluoro-4-(hydroxymethyl)-1-tosyl-1H-indol- 5-yl)oxy)pyridine-2-carbimidothioate | 438 |
| 55-7 | 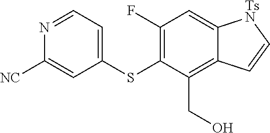 [SMILES: Cc1ccc(S(=O)(=O)n2ccc3c(CO)c(Sc4ccnc(C#N)c4)c(F)cc32)cc1] | 4-((6-fluoro-4-(hydroxymethyl)-1-tosyl-1H-indol-5- yl)thio)picolinonitrile | 454 |

Intermediate 55A: 2-Fluoro-5-((6-fluoro-1-(tetrahydro-2H-pyran-2-yl)-4-vinyl-1H-benzo[d]imidazol-5-yl)oxy)benzonitrile

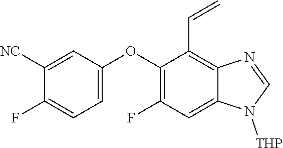

To a stirred and degassed solution of 5-((4-bromo-6-fluoro-1-(tetrahydro-2H-pyran-2-yl)-1H-benzo[d]imidazol-5-yl)oxy)-2-fluorobenzonitrile (Intermediate 54, 6.6 g, 15.3 mmol) and 4,4,5,5-tetra-methyl-2-vinyl-1,3,2-dioxaborolane (4.72 g, 30.7 mmol) in dioxane (120 mL) and water (30 mL) was added cesium carbonate (9.9 g, 30.7 mmol) and 1,1′-bis(diphenylphosphino)ferrocene-palladium (II) dichloride dichloromethane complex (1:1, 1.25 g, 1.53 mmol). The mixture was stirred at 100° C. overnight, cooled to room temperature and diluted with ethyl acetate (300 mL). The solution was washed with brine, dried over sodium sulfate, and concentrated. The residue was purified by automated flash chromatography (120 g silica gel column, eluting with 0-60% ethyl acetate in petroleum ether) to give the title compound (5 g, 86%) as a solid. MS (ESI): 382 m/z [M+H]⁺, retention time: 2.20 minutes, purity: 88% (214 nm) (LC-MS method 011).

The following intermediate was prepared based on the procedures described for Intermediate 55A

| Inter. | | | MS m/z |
|---|---|---|---|
| No. | Structure | Name | [M + H]⁺ |
| 55A-1 | 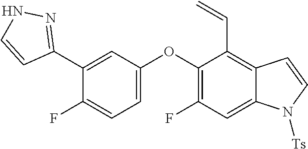 [SMILES: C=Cc1c(Oc2ccc(F)c(-c3cc[nH]n3)c2)c(F)cc2c1ccn2S(=O)(=O)c1ccc(C)cc1] | 6-fluoro-5-(4-fluoro-3-(1H-pyrazol-3-yl)phenoxy)-1- tosyl-4-vinyl-1H-indole | 492 |
| 55A-2 | 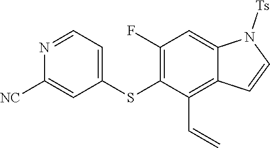 [SMILES: C=Cc1c(Sc2ccnc(C#N)c2)c(F)cc2c1ccn2S(=O)(=O)c1ccc(C)cc1] | 4-((6-fluoro-1-tosyl-4-vinyl-1H-indol-5- yl)thio)picolinonitrile | 450 |

Intermediate 55B: 2-Fluoro-5-((6-fluoro-4-formyl-1-(tetrahydro-2H-pyran-2-yl)-1H-benzo[d]imidazol-5-yl)oxy)benzonitrile

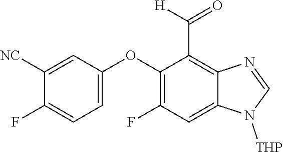

To a solution of 2-fluoro-5-((6-fluoro-1-(tetrahydro-2H-pyran-2-yl)-4-vinyl-1H-benzo[d]imidazol-5-yl)oxy)benzonitrile (Intermediate 55A, 4.4 g, 11.5 mmol) in tetrahydrofuran (200 mL was added osmium tetroxide (0.1% in water) (0.44 ML followed by a solution of sodium periodate (7.4 g, 34.6 mmol) in water (100 ml. The mixture was stirred at room temperature over the weekend. The mixture was diluted with water (500 mL), extracted with ethyl acetate (3×200 mL). The combined organic extracts were washed with brine, dried over sodium sulfate, and concentrated to give the crude title compound (4.4 g, 99%) as a solid, which was used in the next step without further purification. MS (ESI): 384 m/z [M+H]⁺, retention time: 2.03 minutes, purity: 51% (214 nm) (LC-MS method 2).

The following intermediate was prepared based on the procedures described for Intermediate 55B

| Inter. | | | MS m/z |
|---|---|---|---|
| No. | Structure | Name | [M + H]⁺ |
| 55B-1 | 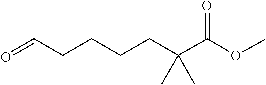 | methyl 2,2-dimethyl- 7-oxoheptanoate | ¹H NMR (400 MHz, CDCl₃) δ 9.76 (t, J = 1.6 Hz, 1H), 3.66 (s, 3H), 2.42 (td, J = 7.6, 1.6 Hz, 2H), 1.65-1.57 (m, 2H), 1.55-1.51 (m, 2H), 1.29-1.23 (m, 2H), 1.16 (s, 6H) ppm. |
| 55B-1 | 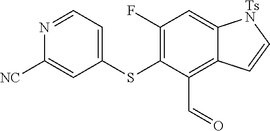 [SMILES: Cc1ccc(S(=O)(=O)n2ccc3c(C=O)c(Sc4ccnc(C#N)c4)c(F)cc32)cc1] | 4-((6-fluoro-4-formyl- 1-tosyl-1H-indol-5- yl)thio)picolinonitrile | 452 |

Intermediate 56: 3-((6-Fluoro-4-vinyl-1H-indol-5-yl)oxy)benzothioamide

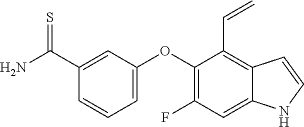 [SMILES: C=Cc1c(Oc2cccc(C(N)=S)c2)c(F)cc2[nH]ccc12]

To a stirred solution of 3-((6-fluoro-4-vinyl-1H-indol-5-yl)oxy)benzonitrile (Intermediate 15-2, 1.6 g, 5.75 mmol) in dimethylformamide (20 mL) was added sodium hydrosulfide (1.93 g, 36.5 mmol), magnesium chloride (1.64 g 17.2 mmol) and water(2.49 g, 138 mmol). The reaction was stirred at room temperature for 1 hour. The mixture was diluted with water (50 mL) and extracted with ethyl acetate (3×50 mL). The combined extracts were washed with water, brine, dried over sodium sulfate, and concentrated. The residue was purified by flash chromatography (80 g silica gel column, eluting with 0-60% ethyl acetate in petroleum ether) to give the title compound (1.5 g, 79%) as a solid. MS (ESI): 313 m/z [M+H]⁺, retention time: 1.29 minutes, purity: 95% (254 nm) (LC-MS method 2).

The following intermediate was prepared based on the procedures described for Intermediate 56.

| Inter. | | | MS m/z |
|---|---|---|---|
| No. | Structure | Name | [M + H]⁺ |
| 56-1 | 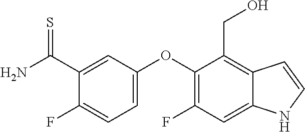 [SMILES: NC(=S)c1cc(Oc2c(F)cc3[nH]ccc3c2CO)ccc1F] | 2-fluoro-5-((6-fluoro-4-(hydroxymethyl)-1H- indol-5-yl)oxy)benzothioamide | 317 [M − H2O + H]⁺ |
| 56-2 | 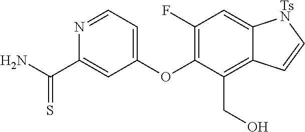 [SMILES: Cc1ccc(S(=O)(=O)n2ccc3c(CO)c(Oc4ccnc(C(N)=S)c4)c(F)cc32)cc1] | 4-((6-fluoro-4-(hydroxymethyl)-1-tosyl-1H- indol-5-yl)oxy)pyridine-2-carbothioamide | 472 |
| 56-3 | 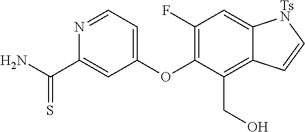 [SMILES: Cc1ccc(S(=O)(=O)n2ccc3c(CO)c(Oc4ccnc(C(N)=S)c4)c(F)cc32)cc1] | 4-((6-fluoro-4-(hydroxymethyl)-1-tosyl-1H- indol-5-yl)thio)pyridine-2-carbothioamide | 488 |
| 56-4 | 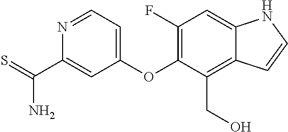 [SMILES: NC(=S)c1cc(Oc2c(F)cc3[nH]ccc3c2CO)ccn1] | 4-((6-fluoro-4-(hydroxymethyl)-1H-indol-5- yl)oxy)pyridine-2-carbothioamide | 318 m/z [M + H]⁺; RT: 1.76 minutes (LC-MS method 2) |
| 56-5 | 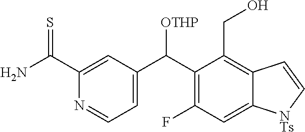 | 4-((6-fluoro-4-(hydroxymethyl)-1-tosyl-1H- indol-5-yl)((tetrahydro-2H-pyran-2- yl)oxy)methyl)pyridine-2-carbothioamide | 570 m/z [M + H]⁺; RT: 2.09 + 2.11 minutes (LC-MS method 2) |
| 56-6 | 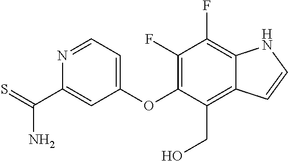 [SMILES: NC(=S)c1cc(Oc2c(F)c(F)c3[nH]ccc3c2CO)ccn1] | 4-((6,7-difluoro-4-(hydroxymethyl)-1H-indol-5- yl)oxy)pyridine-2-carbothioamide | 336 m/z [M + H]⁺; RT: 1.22 minutes (LC-MS method 2) |

Intermediate 57: 2-Fluoro-5-((6-fluoro-1-tosyl-4-vinyl-1H-indol-5-yl)oxy)benzamide

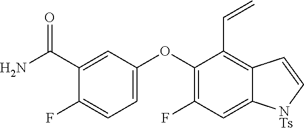 [SMILES: C=Cc1c(Oc2ccc(F)c(C(N)=O)c2)c(F)cc2c1ccn2S(=O)(=O)c1ccc(C)cc1]

To a stirred and cooled (0° C.) solution of 2-fluoro-5-((6-fluoro-4-vinyl-1H-indol-5-yl)oxy)benzamide (Intermediate 57A, 2.7 g, 8.59 mmol) in tetrahydrofuran (96.4 mL) was added sodium hydride (395 mg, 10.3 mmol). The mixture was stirred for 30 minutes, treated with 4-methylbenzenesulfonyl chloride (2.46 mg, 12.9 mmol). The reaction was stirred at room temperature for 16 hours, quenched with water (80 mL), and extracted with ethyl acetate (3×100 mL). The combined organic layers were dried over sodium sulfate, and concentrated. The residue was purified by silica gel column (petroleum ether: ethyl acetate=10:1) to give the title compound (3.3 g, 75%) as a yellow solid. MS (ESI): 469 m/z [M+H]⁺, retention time: 2.22 minutes, purity: 92% (214 nm) (LC-MS method 2).

Intermediate 57A: 2-Fluoro-5-((6-fluoro-4-vinyl-1H-indol-5-yl)oxy)benzamide

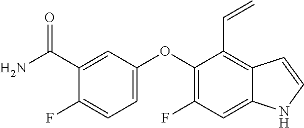 [SMILES: C=Cc1c(Oc2ccc(F)c(C(N)=O)c2)c(F)cc2[nH]ccc12]

To a stirred solution of 2-fluoro-5-((6-fluoro-4-vinyl-1H-indol-5-yl)oxy)benzonitrile (Intermediate 15-1 3.1 g, 10.5 mmol) in ethanol (103 mL) and water (31 mL) was added hydrogen peroxide (8.3 g, 7.32 mmol) and sodium hydroxide (1.26 g, 31.4 mmol). The mixture was stirred at room temperature for 16 hours, then diluted with brine (100 mL), and acidified with 1 M hydrochloric acid (20 mL). The mixture was extracted with ethyl acetate (3×100 mL). The combined organic layers were washed with saturated sodium bicarbonate, brine, dried over sodium sulfate and concentrated. The obtained crude title compound (2.7 g, 75%, yellow solid) was used for the next step without further purification. MS (ESI): 315 m/z [M+H]⁺, retention time: 1.98 minutes, purity: 91% (214 nm) (LC-MS method 2).

Intermediate 58: (6-Fluoro-5-(4-fluoro-3-(1H-1,2,4-triazol-3-yl)phenoxy)-1-tosyl-1H-indol-4-yl)methanol

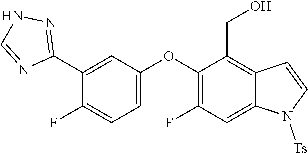 [SMILES: Cc1ccc(S(=O)(=O)n2ccc3c(CO)c(Oc4ccc(F)c(-c5nc[nH]n5)c4)c(F)cc32)cc1]

A mixture of 2-fluoro-5-((6-fluoro-4-(hydroxymethyl)-1-tosyl-1H-indol-5-yl)oxy)benzamide (Intermediate 55-1, 2.3 g, 4.87 mmol) in N,N-dimethylformamide dimethyl acetal (43.4 mL) was stirred at 80° C. for 5 hours. The solvent was removed. The residue was dissolved in acetic acid (43.4 mL), treated with hydrazine (172 mg, 5.35 mmol). The reaction mixture was stirred at 80° C. for 2 hours. The solvent was removed. The residue was partitioned between 50 mL of water and 80 mL of ethyl acetate. The separated organic layer, combined with two additional ethyl acetate extracts (2×80 mL), was washed with saturated sodium bicarbonate, brine, dried over sodium sulfate, and concentrated. The residue was purified by silica gel chromatography (petroleum ether: ethyl acetate=1:1) to give the title compound (1.2 g, 46%) as a white solid. MS (ESI): 497 m/z [M+H]⁺, retention time: 1.28 minutes, purity: 93% (214 nm) (LC-MS method 2).

Intermediate 59: 1-bromo-3-(1-bromo-4-((2-methylbut-3-yn-2-yl)oxy)butyl)benzene

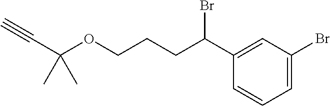 [SMILES: C#CC(C)(C)OCCCC(Br)c1cccc(Br)c1]

To a stirred and cooled (0° C.) solution of N-bromosuccinimide (3.89 g, 21.8 mmol) in dichloromethane (50 mL) was added triphenyl phosphine (5.73 g, 1.93 mmol) in dichloromethane (50 mL). The mixture was stirred for 0.5 hours, then treated with a solution of 1-(3-bromophenyl)-4-((2-methylbut-3-yn-2-yl)oxy)butan-1-ol (Intermediate 59D, 3.4 g, 10.9 mmol) in dichloromethane (20 mL) and stirred for another 4 hours and quenched with saturated ammonium chloride (100 mL) was added and the mixture was extracted with ethyl acetate (3×100 mL). The combined organic phases were washed with brine, dried over sodium sulfate, and concentrated. The residue was purified by column chromatography on silica gel using ethyl acetate/petroleum ether (10:90) to afford the title compound as an oil (2.3 g, 56%). ¹H NMR (400 MHz, CDCl₃) δ 7.55 (t, J=1.8 Hz, 1H), 7.43-7.39 (m, 1H), 7.34-7.30 (m, 1H), 7.23-7.17 (m, 1H), 4.97-4.91 (m, 1H), 3.58-3.54 (m, 2H), 2.41 (s, 1H), 2.29-2.23 (m, 2H), 1.76-1.71 (m, 1H), 1.63-1.57 (m, 1H), 1.45 (s, 6H) ppm.

The following intermediates were prepared based on the procedures described for Intermediate 59 and/or for Intermediate 59A to 59D.

| Inter. | | | MS m/z |
|---|---|---|---|
| No. | Structure | Name | [M + H]⁺ |
| 59-1 | 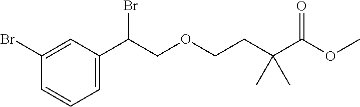 [SMILES: COC(=O)C(C)(C)CCOCC(Br)c1cccc(Br)c1] | methyl 7-bromo-7-(3- bromophenyl)-2,2- dimethylheptanoate | 407 |
| 59-2 | 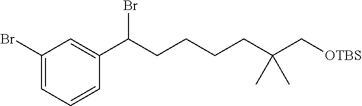 [SMILES: CC(C)(CCCCC(Br)c1cccc(Br)c1)CO[Si](C)(C)C(C)(C)C] | ((7-bromo-7-(3- bromophenyl)-2,2- dimethylheptyl)oxy)(tert- butyl)dimethylsilane | ¹H NMR (400 Mhz, CDCl₃) δ 7.53 (s, 1H), 7.42 (d, J = 7.2 Hz, 1H), 7.28 (s, 1H), 7.25-7.21 (m, 1H), 4.66 (t, J = 7.6 Hz, 1H), 3.22 (s, 2H), 1.80-1.67 (m, 2H), 1.29-1.17 (m, 6H), 0.90 (s, 9H), 0.81 (s, 6H), 0.03 (s, 6H) ppm |
| 59-3 | 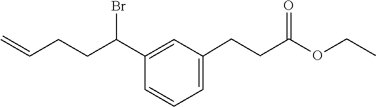 [SMILES: C=CCCC(Br)c1cccc(CCC(=O)OCC)c1] | ethyl 3-(3-(1-bromopent-4- en-1-yl)phenyl)propanoate | ¹H NMR (400 Mhz, CDCl₃) δ 7.26-7.17 (m, 3H), 7.15-7.10 (m, 1H), 5.84-5.71 (m, 1H), 5.09-5.00 (m, 2H), 4.96-4.91 (m, 1H), 4.13 (q, J = 7.2 Hz, 2H), 2.95 (t, J = 7.6 Hz, 2H), 2.62 (t, J = 7.6 Hz, 2H), 2.42-2.14 (m, 4H), 1.23 (t, J = 7.2 Hz, 3H) ppm |

Intermediate 59A: 3-((2-Methylbut-3-yn-2-y)oxy)propanal

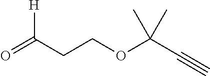

To a stirred solution of 3-((2-methylbut-3-yn-2-yl)oxy)propan-1-ol (Intermediate 24B, 14 g, 98 mmol) in dichloromethane (200 mL) was added pyridinium chlorochromate (42.4 g, 197 mmol) and silica gel (100-200 m, 42 g). The resulting mixture was stirred for 3 hours, diluted with hexanes (200 mL) and filtered. The filtrated was concentrated to give the crude title compound (11 g, 80%), which was used for next step without further purification. ¹H NMR (400 MHz, CDCl₃) δ 9.79 (t, J=1.9 Hz, 1H), 3.92 (t, J=6.1 Hz, 2H), 2.65 (td, J=6.1, 1.9 Hz, 2H), 2.43 (s, 1H), 1.47 (s, 6H) ppm.

The following intermediate was prepared based on the procedures described for Intermediate 59A.

| Inter. | | | MS m/z |
|---|---|---|---|
| No. | Structure | Name | [M + H]⁺ |
| 59A-1 | 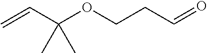 | 3-((2-methylbut-3-en- 2-yl)oxy)propanal | ¹H NMR (400 Mhz, CDCl₃) δ 9.77 (t, J = 2.0 Hz, 1H), 5.85-5.77 (m, 1H), 5.16-5.12 (m, 2H), 3.65 (t, J = 6.4 Hz, 2H), 2.59 (td, J = 6.4, 2.0 Hz, 2H), 1.27 (s, 6H) ppm |

Intermediate 59B: (E)-1-Methoxy-4-((2-methylbut-3-yn-2-yl)oxy)but-1-ene

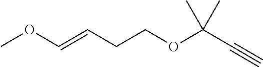

To a stirred and cooled (0° C.) solution of (methoxymethyl)(triphenyl)phosphonium bromide (56.1 g, 145 mmol) in tetrahydrofuran (200 mL) was added sodium bis(trimethylsilyl)amide (2 M in tetrahydrofuran, 78.5 mL, 157 mmol) dropwise. The mixture was stirred at 0° C. for 0.5 hours, then treated with 3-((2-methylbut-3-yn-2-yl)oxy)propanal (Intermediate 57A, 11 g, 78.5 mmol) in tetrahydrofuran (40 mL) dropwise. The mixture was stirred at room temperature for 4 hours, quenched with saturated ammonium chloride (200 mL), and extracted with ethyl acetate (2×200 mL). The combined organic phases were washed with water, brine, dried over sodium sulfate, and concentrated. The residue was purified by column chromatography on silica gel, eluted with ethyl acetate/petroleum ether (5:95) to afford the title compound as oil (3.3 g, 25%). ¹H NMR (400 MHz, CDCl₃) δ 6.25 (d, J=12.7 Hz, 1H), 4.62 (dt, J=12.7, 7.4 Hz, 1H), 3.43-3.35 (m, 5H), 2.28 (s, 1H), 2.11-2.00 (m, 2H), 1.35 (s, 6H) ppm.

Intermediate 59C: 4-((2-Methylbut-3-yn-2-yl)oxy)butanal

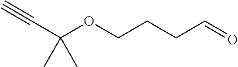

To a stirred solution of (E)-1-methoxy-4-((2-methylbut-3-yn-2-yl)oxy)but-1-ene (Intermediate 59B, 3 g, 17.8 mmol) in tetrahydrofuran (30 mL) was added concentrate hydrochloric acid (3 mL). The mixture was stirred at 0° C. for 20 minutes, then neutralized with saturated sodium bicarbonate (50 mL), and extracted with ethyl acetate (3×50 mL). The combined organic phases were washed with water, brine (50 mL), dried over sodium sulfate, and concentrated to give the crude title compound (2.8 g, 99%). ¹H NMR (400 MHz, CDCl₃) δ 9.78 (s, 1H), 3.64-3.55 (m, 2H), 2.59-2.49 (m, 2H), 2.40 (s, 1H), 1.90 (dt, J=12.0, 6.0 Hz, 2H), 1.45 (s, 6H) ppm.

Intermediate 59D: 1-(3-Bromophenyl)-4-((2-methylbut-3-yn-2-yl)oxy)butan-1-ol

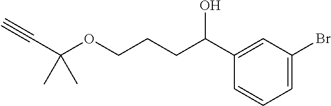 [SMILES: C#CC(C)(C)OCCCC(O)c1cccc(Br)c1]

To a stirred and cooled (−78° C.) solution of 1,3-dibromobenzene (11.5 g, 48.6 mmol) in tetrahydrofuran (50 mL) was added n-butyllithium (2.5 M in hexane, 19.5 mL, 48.6 mmol), the mixture was stirred at this temperature for 2 hours, then treated with 4-((2-methylbut-3-yn-2-yl)oxy)butanal (Intermediate 59C, 2.5 g, 16.2 mmol) in tetrahydrofuran (10 mL). The mixture was stirred for another 1 hour and quenched with saturated ammonium chloride (100 mL). After warming to room temperature, the solution was extracted with ethyl acetate (3×100 mL). The combined organic phases were washed with brine, dried over sodium sulfate, and concentrated. The residue was purified by column chromatography on silica gel column using ethyl acetate/petroleum ether (10:90) to afford the desired product as an oil (2 g, 40%). ¹H NMR (400 MHz, CDCl₃) δ 7.53 (s, 1H), 7.38 (d, J=7.8 Hz, 1H), 7.28-7.24 (m, 1H), 7.20 (t, J=7.8 Hz, 1H), 4.73-4.68 (m, 1H), 3.67-3.53 (m, 2H), 3.15 (d, J=3.5 Hz, 1H), 2.42 (s, 1H), 1.96-1.78 (m, 2H), 1.74-1.66 (m, 2H), 1.49 (s, 6H) ppm.

The following intermediate was prepared based on the procedures described for Intermediate 59D.

| Inter. | | | MS m/z |
|---|---|---|---|
| No. | Structure | Name | [M + H]⁺ |
| 59D-1 | 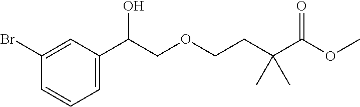 [SMILES: COC(=O)C(C)(C)CCOCC(O)c1cccc(Br)c1] | methyl 7-(3-bromophenyl)-7-hydroxy-2,2- dimethylheptanoate | 343, 345 |

Intermediate 60: 5-((4,6-Difluoro-1H-indol-5-yl)oxy)-2-fluorobenzonitrile

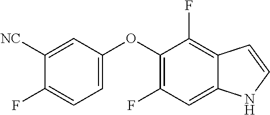 [SMILES: N#Cc1cc(Oc2c(F)cc3[nH]ccc3c2F)ccc1F]

A mixture of 5-(4-amino-2,6-difluoro-3-((trimethylsilyl)ethynyl)phenoxy)-2-fluorobenzonitrile (Intermediate 60D, 900 mg, 2.50 mmol) and copper (I) iodide (950 mmol, 5.0 mmol) in dimethylformamide (5 mL) was heated at 100° C. in a glove box overnight. The insoluble material was removed by suction filtration and the filtrate was diluted with ethyl acetate (100 mL). The organic phase was washed with brine, dried over sodium sulfate, filtered and concentrated. The resulting residue was purified by silica gel column chromatography (20% ethyl acetate in petroleum ether) to afford the title compound as a yellow solid (440 mg, 59%). ¹H NMR (400 MHz, DMSO-d₆) δ 11.64 (s, 1H), 7.63-7.61 (m, 1H), 7.51-7.47 (m, 2H), 7.39-7.35 (m, 1H), 7.32 (d, J=10.4 Hz, 1H), 6.58-6.57 (m, 1H) ppm. MS m/z: 287 [M−1]⁻.

The following intermediates were prepared utilizing the procedures described for Intermediate 60 and/or Intermediates 60A to 60D.

| Inter. | | | |
|---|---|---|---|
| No. | Structure | Name | LCMS-(m/z) |
| 60-1 | 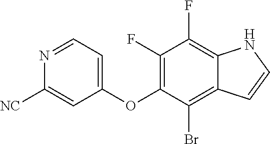 [SMILES: N#Cc1cc(Oc2c(F)c(F)c3[nH]ccc3c2Br)ccn1] | 4-((4-bromo-6,7-difluoro-1H-indol-5-yl)oxy)picolinonitrile | 350, 352 [M + H]⁺; |

Intermediate 60A: 5-(2,6-Difluoro-4-nitrophenoxy)-2-fluorobenzonitrile

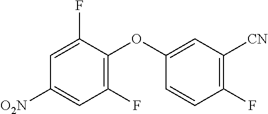 [SMILES: N#Cc1cc(Oc2c(F)cc([N+](=O)[O-])cc2F)ccc1F]

To a stirred solution of 1,2,3-trifluoro-5-nitrobenzene (1.10 g, 6.21 mmol) in DMF (5 mL) were added potassium carbonate (1.71 g, 12.4 mmol) and 2-fluoro-5-hydroxybenzonitrile (936 mg, 6.83 mmol). The resulting mixture was heated at 100° C. for two hours, cooled to room temperature and quenched with water (20 mL). The yellow precipitate was collected by filtration, washed with water and dried to afford the title compound (1.70 g, 94%). ¹H NMR (400 MHz, CDCl₃) δ 8.06-8.01 (m, 2H), 7.30-7.20 (m, 3H) ppm.

Intermediate 60B: 5-(4-Amino-2,6-difluorophenoxy)-2-fluorobenzonitrile

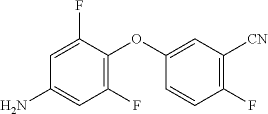 [SMILES: N#Cc1cc(Oc2c(F)cc(N)cc2F)ccc1F]

To a suspension of 5-(2,6-difluoro-4-nitrophenoxy)-2-fluorobenzonitrile (Intermediate 60A, 1.70 g, 5.78 mmol) in ethanol (30 mL) were added a solution of ammonium chloride (2.45 g, 46.2 mmol) in water (10 mL) and iron power (1.48 g, 23.1 mmol). The reaction mixture was heated at reflux for four hours, the insoluble material was removed by filtration and the filtrate was concentrated. The resulting residue was dissolved in ethyl acetate (100 mL), washed with water (30 mL×3) and brine (30 mL), dried over sodium sulfate, filtered, and concentrated to afford the title compound as a yellow solid (1.60 g, 98%). MS m/z: 263 [M−1]⁻.

Intermediate 60C: 5-(4-Amino-2,6-difluoro-3-iodophenoxy)-2-fluorobenzonitrile

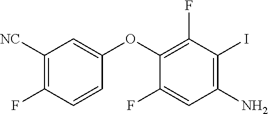 [SMILES: N#Cc1cc(Oc2c(F)cc(N)c(I)c2F)ccc1F]

A solution of 5-(4-amino-2,6-difluorophenoxy)-2-fluorobenzonitrile (Intermediate 60B, 1.60 g, 6.06 mmol) and NIS (1.36 g, 6.06 mmol) in acetic acid was stirred at 30° C. for one hour and then concentrated under reduced pressure. The resulting residue was purified by silica gel column chromatography (20% ethyl acetate in petroleum ether) to afford the title compound (2.08 g, 87%) as a yellow solid. MS m/z: 389 [M−1]⁻.

Intermediate 60D: 5-(4-Amino-2,6-difluoro-3-((trimethylsilyl)ethynyl)phenoxy)-2-fluorobenzonitrile

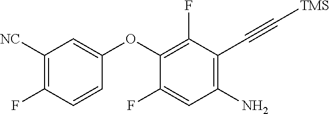 [SMILES: C[Si](C)(C)C#Cc1c(N)cc(F)c(Oc2ccc(F)c(C#N)c2)c1F]

To a stirred solution of 5-(4-amino-2,6-difluoro-3-iodophenoxy)-2-fluorobenzonitrile (Intermediate 60C, 1.0 g, 2.56 mmol) in DMF (15 mL) were added Pd(dppf)Cl₂ (182 mg, 0.26 mmol), CuI (48 mg, 0.26 mmol), triethylamine (0.54 mL, 3.84 mmol) and ethynyltrimethylsilane (0.47 mL, 3.33 mmol). The reaction mixture was stirred at 30° C. for three hours under nitrogen atmosphere, quenched with water (30 mL) and extracted with ethyl acetate (20 mL×3). The combined organic extracts were washed with brine (10 mL), dried over sodium sulfate, filtered and concentrated. The resulting residue was purified by silica gel column chromatography (20% ethyl acetate in petroleum ether) to afford the title compound as a yellow solid (900 mg, 97%). MS m/z: 359 [M−1]⁻.

Intermediate 61: 2-(3-(2-ethoxy-2-oxoethyl)phenyl)-8-hydroxy-2,7,7-trimethyloctanoic acid

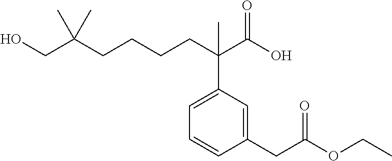 [SMILES: CCOC(=O)Cc1cccc(C(C)(CCCCC(C)(C)CO)C(=O)O)c1]

To a stirred solution of 2-(trimethylsilyl)ethyl 8-((tert-butyldimethylsilyl)oxy)-2-(3-(2-ethoxy-2-oxoethyl)phenyl)-2,7,7-trimethyloctanoate (Intermediate 61B, 4 g, 6.91 mmol) in tetrahydrofuran (100 mL) was added tetrabutylammonium fluoride (34.5 mL, 1 N in tetrahydrofuran, 34.5 mmol). The mixture was stirred at room temperature for 12 hours, then acidified to pH ˜3 with 1 N hydrochloric acid. The solution was extracted with ethyl acetate (3×50 mL). The combined organic layers were washed with brine (30 mL), dried over sodium sulfate, and concentrated. The residue was purified by silica gel column (eluting with petroleum ether/ethyl acetate=5:1) to give the title compound (2.1 g, 83%) as yellow oil. MS (ESI): 365 m/z [M+H]⁺, retention time: 2.01 minutes, purity: 92% (214 nm) (LC-MS method 2).

Intermediate 61A: 2-(Trimethylsilyl)ethyl 2-(3-bromophenyl)-8-((tert-butyldimethylsilyl)oxy)-2,7,7-trimethyloctanoate

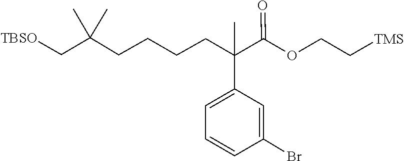 [SMILES: CC(C)(CCCCC(C)(C(=O)OCC[Si](C)(C)C)c1cccc(Br)c1)CO[Si](C)(C)C(C)(C)C]

To a stirred and cooled (−78° C.) solution of 2-(trimethylsilyl)ethyl 2-(3-bromophenyl)propanoate (Intermediate 7A-23, 10 g, 30.4 mmol) in tetrahydrofuran (200 mL) was added lithium diisopropylamide (16.7 mL, 2 N in tetrahydrofuran, 33.4 mmol). The mixture was stirred at −78° C. for one hour and then treated with tert-butyl((6-iodo-2,2-dimethylhexyl)oxy)dimethylsilane (Intermediate 27, 12.4 g, 33.4 mmol) dropwise. The mixture was stirred at 15˜20° C. for 12 hours, then quenched with ammonium chloride (100 mL). The solution was extracted with ethyl acetate (3×300 mL). The combined organic layers were washed with brine, dried over sodium sulfate, and concentrated. The residue was purified by silica gel column (eluting with petroleum ether/ethyl acetate=10:1) to give the title compound (14 g, 80%) as yellow oil. MS (ESI): No molecular weight peak detected, retention time: 2.58 minutes, purity: 45% (214 nm) (LC-MS method 5).

Intermediate 61B: 2-(Trimethylsilyl)ethyl 8-((tert-butyldimethylsilyl)oxy)-2-(3-(2-ethoxy-2-oxoethyl)phenyl)-2,7,7-trimethyloctanoate

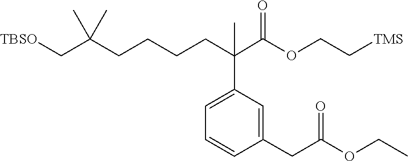 [SMILES: CCOC(=O)Cc1cccc(C(C)(CCCCC(C)(C)CO[Si](C)(C)C(C)(C)C)C(=O)OCC[Si](C)(C)C)c1]

To a stirred and degassed solution of 2-(trimethylsilyl)ethyl 2-(3-bromophenyl)-8-((tert-butyldimethylsilyl)oxy)-2,7,7-trimethyloctanoate (Intermediate 61A, 10 g, 17.5 mmol) in dimethylformamide (30 mL) was added zinc bromide (7.88 g, 35 mmol), ethyl 2-tributylstannylacetate (9.89 g, 26.2 mmol) and dichlorobis(tri-o-tolylphosphine)palladium (II) (1.37 g, 1.75 mmol). The mixture was stirred at 100° C. for 12 hours. The mixture was partitioned between water (150 mL) and ethyl acetate (200 mL). The separated organic layer, combined with two additional ethyl acetate extracts (2×200 mL), was washed with brine, dried over sodium sulfate, and concentrated. The residue was purified by silica gel column (eluting with petroleum ether/ethyl acetate=1:1) to give the title compound (4 g, 40%) as yellow oil. MS (ESI): No molecular weight peak shown, retention time: 1.87 minutes, purity: 80% (254 nm) (LC-MS method 2).

Intermediate 62: 3-Acetoxy-2-(3-bromophenyl)-2-methylpropanoic acid

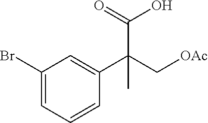 [SMILES: CC(=O)OCC(C)(C(=O)O)c1cccc(Br)c1]

To a stirred solution of methyl 2-(3-bromophenyl)-3-hydroxy-2-methylpropanoate (Intermediate 62A, 3.9 g, 14.3 mmol) in tetrahydrofuran (120 mL) and methanol (60 mL) was added lithium hydroxide in water (1 M) (60 mL). The mixture was stirred at 40° C. for four hours and concentrated. The residue was acidified with 1 N hydrochloric acid to pH ˜4. The solution was extracted with ethyl acetate (3×60 mL). The combined organic extracts were dried over sodium sulfate and concentrated. The residue was dissolved in dichloromethane (30 mL), treated with acetic anhydride (2.19 g, 21.4 mmol) and triethylamine (4 mL, 28.6 mmol). The mixture was stirred at room temperature for four hours, then diluted with dichloromethane (30 mL), washed with 1N hydrochloric acid, brine, dried over sodium sulfate, and concentrated. The residue was purified by automated flash chromatography (40 g silica gel column, eluting with 0-50% ethyl acetate in petroleum ether) to give the title compound (3.7 g, 86%) as an oil. ¹H NMR (400 MHz, CDCl₃) δ 7.53 (t, J=1.8 Hz, 1H), 7.47-7.43 (m, 1H), 7.35-7.31 (m, 1H), 7.24 (t, J=7.9 Hz, 1H), 4.56 (d, J=10.9 Hz, 1H), 4.35 (d, J=10.9 Hz, 1H), 2.05 (s, 3H), 1.66 (s, 3H).

Intermediate 62A: Methyl 2-(3-bromophenyl)-3-hydroxy-2-methylpropanoate

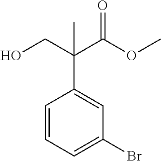

To a stirred and cooled (−78° C.) solution of methyl 2-(3-bromophenyl)propanoate (10.0 g, 41.1 mmol) in tetrahydrofuran (100 mL) was added lithium diisopropylamide (2 M in tetrahydrofuran, 24.7 mL, 49.4 mmol). The mixture was stirred at −78° C. for one hour, then treated with para-formaldehyde (2.47 g, 82.2 mmol). The reaction was stirred at room temperature for four hours, quenched with water (200 mL), and extracted with ethyl acetate (2×100 mL). The combined organic extracts were washed with brine, dried over sodium sulfate, and concentrated. The residue was purified by automated flash chromatography (80 g silica gel column, eluting with 0-50% ethyl acetate in petroleum ether) to give the title compound (6.3 g, 56%) as colorless oil. ¹H NMR (400 MHz, CDCl₃) δ 7.48-7.27 (m, 4H), 4.07 (d, J=11 Hz, 1H), 3.77 (d, J=11 Hz, 1H), 3.72 (s, 3H), 1.60 (s, 3H).

Intermediate 63: 2-Fluoro-5-((6-fluoro-4-iodo-1H-indol-5-yl)oxy)benzonitrile (Mixture)

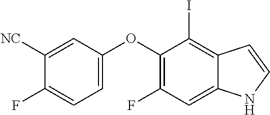 [SMILES: N#Cc1cc(Oc2c(F)cc3[nH]ccc3c2I)ccc1F]

In a glove box, to an Acid Digestion Bomb (100 mL) was added 5-((4-bromo-6-fluoro-1H-indol-5-yl)oxy)-2-fluorobenzonitrile (Intermediate 3, 20 g, 57.3 mmol), sodium iodide (17.2 g, 115 mmol), copper (I) iodide (1.09 g, 5.73 mmol), 1,4-dioxane (100 mL) and N,N′-dimethylethylenediamine (1.23 mL, 11.5 mmol). The reaction was heated to 120° C. for 3 days, then diluted with ethyl acetate (500 mL). The solution was washed with water, brine, dried over sodium sulfate, and concentrated. The residue was purified by automated flash chromatography (120 g silica gel column, eluting with 0-30% ethyl acetate in petroleum ether) to give the title compound (20.6 g) as a solid. ¹H NMR (400 MHz, CDCl₃) δ 8.63-8.55 (m, 1H), 7.35-7.31 (m, 1H), 7.28-7.11 (m, 3H), 7.02 (dd, J=7.7, 4.5 Hz, 1H), 6.63 (t, J=2.3 Hz, 0.54H), 6.52 (t, J=2.3 Hz, 0.45H) ppm. (Mixture of bromo (starting material) and Iodo product, almost 1:1)

Intermediate 64: Tert-Butyl 3-((3-(5-(3-cyano-4-fluorophenoxy)-6-fluoro-1H-indol-4-yl)prop-2-yn-1-yl)oxy)propanoate

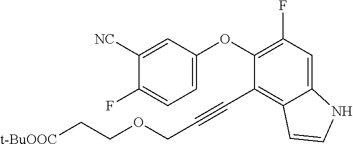

To a stirred and degassed solution of 2-fluoro-5-((6-fluoro-4-iodo-1H-indol-5-yl)oxy)benzonitrile (Intermediate 63, contained 54% of 5-((4-bromo-6-fluoro-1H-indol-5-yl)oxy)-2-fluorobenzonitrile, 20.6 g, 25.5 mmol) in dimethylformamide (200 mL) was added tert-butyl 3-prop-2-ynoxypropanoate (Intermediate 24A-1, 9.39 g, 51 mmol), bis(triphenylphosphine)palladium(II) dichloride (3.72 g, 5.1 mmol), copper (I) iodide(1.94 g, 10.2 mmol) and triethylamine (71 mL, 510 mmol). The reaction was stirred at room temperature overnight, then diluted with water (500 mL). The solution was extracted with ethyl acetate (4×150 mL). The combined extracts were washed with brine, dried over sodium sulfate, and concentrated. The residue was purified twice by automated flash chromatography (330 g silica gel column, eluting with 0-35% ethyl acetate in petroleum ether) to give the title compound (8.7 g, 33.6% based on 20 g of 5-((4-bromo-6-fluoro-1H-indol-5-yl)oxy)-2-fluorobenzonitrile as a solid. MS (ESI): 475 m/z [M+Na]⁺, retention time: 2.19 minutes, purity: 95% (254 nm) (LC-MS method 2).

Intermediate 65: 2-(3-Bromophenyl)-2,7-dimethyl-7-(2-oxooxazolidin-4-yl)octanoic acid

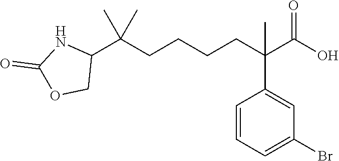 [SMILES: CC(CCCCC(C)(C)C1COC(=O)N1)(C(=O)O)c1cccc(Br)c1]

To a stirred solution of 2-(3-bromophenyl)-8-((tert-butoxycarbonyl)amino)-9-hydroxy-2,7,7-trimethylnonanoic acid (Intermediate 65F, 1.4 g, 2.88 mmol) in tetrahydrofuran (10 mL) was added sodium hydride (60% in mineral oil, 345 mg, 8.63 mmol). The mixture was stirred at room temperature for 16 hours, quenched with 1 N hydrochloric acid to pH ˜5, and extracted with ethyl acetate (3×50 mL). The combined organic extracts were dried over sodium sulfate and concentrated. The residue was purified by silica gel column chromatography (eluting with petroleum ether/ethyl acetate=2/1) to give the title compound (1.1 g, 93%) as a yellow oil. MS (ESI): 412,414 m/z [M+H]⁺, retention time: 2.00 minutes, purity: 60% (214 nm) (LC-MS method 9).

Intermediate 65A: Methyl 8-amino-2-(3-bromophenyl)-8-cyano-2,7,7-trimethyloctanoate

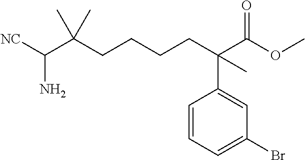 [SMILES: COC(=O)C(C)(CCCCC(C)(C)C(N)C#N)c1cccc(Br)c1]

To a solution of methyl 2-(3-bromophenyl)-2,7,7-trimethyl-8-oxooctanoate (Intermediate 28B-1, 12.0 g, 0.0325 mol) in methanol (30 mL) and water (15 mL) was added ammonia (28% in water, 2.28 g, 65 mmol), potassium cyanide (2.22 g, 34.1 mol) and ammonium chloride (1.91 g, 35.7 mmol). The mixture was stirred at 70° C. for 16 hours, cooled to room temperature, and extracted with ethyl acetate (3×100 mL). The combined organic phases were dried over sodium sulfate and concentrated. The residue was purified by automated silica gel column chromatography (eluting with petroleum ether: ethyl acetate=8:1 to 2:1) to give the title compound (7.0 g, 55%) as colorless oil. MS (ESI): 395, 397 m/z [M+H]⁺, retention time: 1.94 minutes, purity: 88% (214 nm) (LC-MS method 9).

Intermediate 65B: 8-Amino-2-(3-bromophenyl)-2,7,7-trimethylnonanedioic acid

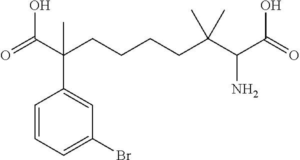 [SMILES: CC(CCCCC(C)(C)C(N)C(=O)O)(C(=O)O)c1cccc(Br)c1]

A mixture of methyl 8-amino-2-(3-bromophenyl)-8-cyano-2,7,7-trimethyloctanoate (Intermediate 65A, 7.0 g, 17.7 mmol) and 12 M hydrochloric acid (50 mL) in acetic acid (10 mL) was stirred at 125° C. for 24 hours and concentrated. The crude title compound (7.0 g, 99%, yellow oil) was used for the next step without further purification. MS (ESI): 400, 402 m/z [M+H]⁺, retention time: 1.63 minutes, purity: 57% (214 nm) (LC-MS method 9).

Intermediate 65C: 2-Amino-8-(3-bromophenyl)-9-methoxy-3,3,8-trimethyl-9-oxononanoic acid

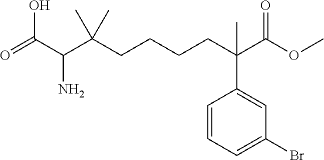 [SMILES: COC(=O)C(C)(CCCCC(C)(C)C(N)C(=O)O)c1cccc(Br)c1]

A mixture of 8-amino-2-(3-bromophenyl)-2,7,7-trimethylnonanedioic acid (Intermediate 65B, 7.0 g, 17.5 mmol), sulfuric acid (concentrated, 1 mL) in methanol (30 mL) was heated at 70° C. for 16 hours. The mixture was concentrated to afford the title compound (7.0 g, 97%, a yellow oil), which was used for the next step without further purification. MS (ESI): 414, 416 m/z [M+H]⁺, retention time: 2.19 minutes, purity: 57% (214 nm) (LC-MS method 4).

Intermediate 65D: 8-(3-Bromophenyl)-2-((tert-butoxycarbonyl)amino)-9-methoxy-3,3,8-trimethyl-9-oxononanoic acid

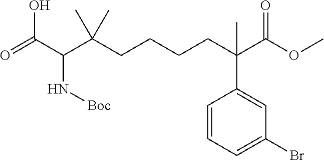 [SMILES: COC(=O)C(C)(CCCCC(C)(C)C(NC(=O)OC(C)(C)C)C(=O)O)c1cccc(Br)c1]

To a stirred solution of 2-amino-8-(3-bromophenyl)-9-methoxy-3,3,8-trimethyl-9-oxononanoic acid (Intermediate 65C, 5.0 g, 13.4 mmol) in tetrahydrofuran (20 mL) and water (10 mL) was added di-tert-butyl dicarbonate (7.0 g, 16.9 mmol) and potassium carbonate (7.0 g, 50.7 mmol). The mixture was stirred at room temperature for 2 hours, acidified to pH ˜4, and extracted with ethyl acetate (3×100 mL). The combined organic phases were dried and concentrated to provide the crude title compound (4.2 g, 48%) as a yellow oil. MS (ESI): 536, 538 m/z [M+Na]⁺, retention time: 2.19 minutes, purity: 57% (214 nm) (LC-MS method 9).

Intermediate 65E: Methyl 2-(3-bromophenyl)-8-((tert-butoxycarbonyl)amino)-9-hydroxy-2,7,7-trimethylnonanoate

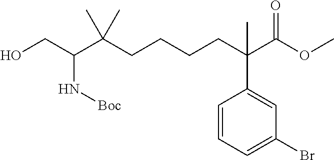 [SMILES: COC(=O)C(C)(CCCCC(C)(C)C(CO)NC(=O)OC(C)(C)C)c1cccc(Br)c1]

To a stirred and cooled (at 0° C.) solution of 8-(3-bromophenyl)-2-((tert-butoxycarbonyl)amino)-9-methoxy-3,3,8-trimethyl-9-oxononanoic acid (Intermediate 65D, 3.3 g, 6.41 mmol) in tetrahydrofuran (10 mL) was added borane dimethyl sulfide complex (2 M in tetrahydrofuran, 9.65 mL, 19.2 mmol). The mixture was stirred at room temperature for 16 hours, then quenched with methanol (10 mL), and concentrated to give the crude title compound (2.5 g, 97.3%) as yellow oil. MS (ESI): 500, 502 m/z [M+H]⁺, retention time: 1.67 minutes, purity: 44% (214 nm) (LC-MS method 9).

Intermediate 65F: 2-(3-Bromophenyl)-8-((tert-butoxycarbonyl)amino)-9-hydroxy-2,7,7-trimethylnonanoic acid

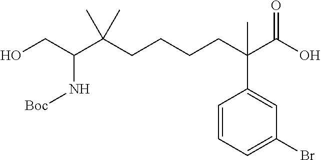 [SMILES: CC(C)(C)OC(=O)NC(CO)C(C)(C)CCCCC(C)(C(=O)O)c1cccc(Br)c1]

To a solution of methyl 2-(3-bromophenyl)-8-((tert-butoxycarbonyl)amino)-9-hydroxy-2,7,7-trimethylnonanoate (Intermediate 65E, 3.3 g, 8.24 mmol) in tetrahydrofuran(10 mL), methanol (10 mL) and water 10 mL) was added lithium hydroxide monohydrate (3.46 g, 82.4 mmol). The mixture was stirred at 50° C. for 16 hours and concentrated. The aqueous residue was acidified with 3 N hydrochloric acid to pH ˜4 and concentrated to dryness. The residue (3.0 g, 7.77 mmol) was dissolved in tetrahydrofuran (20 mL) and water (10 mL) and treated with di-tert-butyl bicarbonate (1.69 g, 7.77 mmol) and potassium carbonate (4.29 g, 1.79 mmol). The mixture was stirred at room temperature for 3 hours, then adjusted pH to ˜6 using 1N hydrochloric acid. The solution was extracted with ethyl acetate (3×50 mL). The combined organic phase was dried over sodium sulfate and concentrated. The crude product was purified by column chromatography (petroleum ether/ethyl acetate=2/1) to give the title compound (1.4 g, 37%) as a yellow oil. MS (ESI): 508, 510 m/z [M+Na]⁺, retention time: 2.13 minutes, purity: 60% (214 nm) (LC-MS method 9).

Intermediate 66: 2-(3-(2-Methoxy-2-oxoethyl)phenyl)-2,7,7-trimethylnon-8-ynoic acid

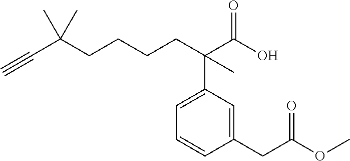 [SMILES: C#CC(C)(C)CCCCC(C)(C(=O)O)c1cccc(CC(=O)OC)c1]

To a stirred and cooled (0° C.) solution of 4-methoxybenzyl 2-(3-(2-methoxy-2-oxoethyl)phenyl)-2,7,7-trimethylnon-8-ynoate (Intermediate 66C, 0.5 g, 1.08 mmol) in dichloromethane (15 mL) was added trifluoroacetic acid (3 mL) dropwise. The mixture was stirred at room temperature for four hours, then partitioned between water (30 mL) and dichloromethane (30 mL). The separated organic layer, combined two additional dichloromethane extracts (2×30 mL), was washed with brine, dried over sodium sulfate and concentrated. The residue was purified by silica gel column (eluting with petroleum ether/ethyl acetate=20:1) to give the title compound (0.2 g, 52%) as yellow oil. MS (ESI): 367 m/z [M+Na]⁺, retention time: 2.14 minutes, purity: 82% (254 nm) (LC-MS method 2).

The following intermediate was prepared based on the procedures described for Intermediate 66.

| Inter. | | | MS m/z |
|---|---|---|---|
| No. | Structure | Name | [M + H]⁺ |
| 66-1 | 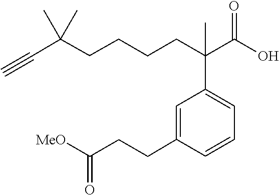 [SMILES: C#CC(C)(C)CCCCC(C)(C(=O)O)c1cccc(CCC(=O)OC)c1] | 2-(3-(3-methoxy-3- oxopropyl)phenyl)-2,7,7- trimethylnon-8-ynoic acid | ¹H NMR (400 Mhz, CDCl₃) δ 7.28-7.19 (m, 3H), 7.09 (d, J = 7.2 Hz, 1H), 3.66 (s, 3H), 2.95 (t, J = 7.6 Hz, 2H), 2.62 (t, J = 8.4 Hz, 2H), 2.09-1.89 (m, 3H), 1.55 (s, 3H), 1.48- 1.33 (m, 4H), 1.24-1.19 (m, 2H), 1.17 (s, 6H) ppm. |

Intermediate 66A: 4-methoxybenzyl 2-(3-(2-ethoxy-2-oxoethyl)phenyl)-8-hydroxy-2,7,7-trimethyloctanoate

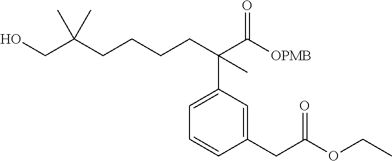

To a stirred solution of 2-(3-(2-ethoxy-2-oxoethyl)phenyl)-8-hydroxy-2,7,7-trimethyloctanoic acid (1 g, 2.74 mmol) and potassium carbonate (Intermediate 61, 0.76 g, 5.48 mmol) in acetone (30 ml) was added 1-(chloromethyl)-4-methoxybenzene (0.59 g, 4.12 mmol). The mixture was stirred at 80° C. for 12 hours and concentrated. The residue was partitioned between water (30 mL) and ethyl acetate (30 mL). The separated organic layer, combined with two additional ethyl acetate extracts (2×30 mL), was washed with brine, dried over sodium sulfate and concentrated. The residue was purified by silica gel column (eluting with petroleum ether/ethyl acetate=5:1) to give the title compound (1 g, 75%) as yellow oil. MS (ESI): 507 m/z [M+Na]⁺, retention time: 2.21 minutes, purity: >99% (254 nm) (LC-MS method 2).

Intermediate 66B: 4-Methoxybenzyl 2-(3-(2-ethoxy-2-oxoethyl)phenyl)-2,7,7-trimethyl-8-oxooctanoate

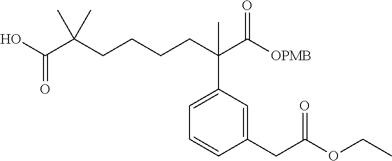

To a stirred mixture of 4-methoxybenzyl 2-(3-(2-ethoxy-2-oxoethyl)phenyl)-8-hydroxy-2,7,7-trimethyloctanoate (Intermediate 66A, 1 g, 2.06 mmol) in dichloromethane (50 mL) was added pyridinium chlorochromate (0.89 g, 4.13 mmol). The mixture was stirred at room temperature for two hours. The mixture was filtered through a pad of Celite. The filtrate was concentrated. The residue was purified by silica gel column (eluting with petroleum ether/ethyl acetate=1:1) to give the title compound (0.8 g, 80%) as yellow oil. MS (ESI): 505 m/z [M+Na]⁺, retention time: 2.34 minutes (LC-MS method 2).

Intermediate 66C: 4-Methoxybenzyl 2-(3-(2-methoxy-2-oxoethyl)phenyl)-2,7,7-trimethylnon-8-ynoate

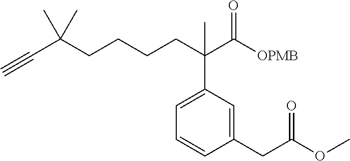

To a stirred solution of 4-methoxybenzyl 2-(3-(2-ethoxy-2-oxoethyl)phenyl)-2,7,7-trimethyl-8-oxooctanoate (Intermediate 66B 0.8 g, 1.66 mmol) in methanol (20 mL) was added 1-diazo-1-dimethoxyphosphoryl-propan-2-one (0.446 g, 2.32 mmol). The mixture was stirred at room temperature for 12 hours and concentrated. The residue was partitioned between water (30 mL) and ethyl acetate (30 mL). The separated organic layer, combined with two additional ethyl acetate extracts (2×30 mL), was washed with brine (30 mL), dried over sodium sulfate, and concentrated. The crude product was purified by silica gel column (eluting with petroleum ether/ethyl acetate=10:1) to give the title compound (0.5 g, 67%) as yellow oil. MS (ESI): 487 m/z [M+Na]⁺, retention time: 2.38 minutes, purity: >99% (254 nm) (LC-MS method 2).

Intermediate 67: 2-Fluoro-5-((6-fluoro-4-(hydroxymethyl)-1H-indol-5-yl)oxy)benzonitrile

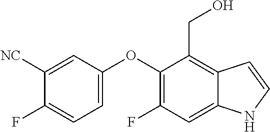 [SMILES: N#Cc1cc(Oc2c(F)cc3[nH]ccc3c2CO)ccc1F]

To a stirred solution of 2-fluoro-5-((6-fluoro-4-(hydroxymethyl)-1-tosyl-1H-indol-5-yl)oxy)-benzonitrile (Intermediate 55-2, 2.00 g, 4.4 mmol) in 30 mL of methanol was added potassium carbonate (1.84 g, 13.2 mmol). The reaction was stirred at 80° C. for 2 hours and concentrated. The residue was partitioned between water (50 mL) and ethyl acetate (50 ml). The separated organic layer, combined with two additional ethyl acetate extracts (2×50 mL), was washed with brine, dried over sodium sulfate and concentrated. The crude product was purified by column chromatography (eluting with petroleum ether/ethyl acetate=2/1) to give the title compound (1.04 g, 79%) as green solid. ¹H NMR (400 MHz, CDCl₃) δ 8.34 (brs, 1H), 7.24 (t, J=2.0 Hz, 1H), 7.16-6.11 (m, 2H), 7.06 (t, J=8.0 Hz, 1H), 6.98-6.94 (m, 1H), 6.68 (t, J=2.4 Hz, 1H), 4.85 (s, 2H) ppm.

Intermediate 68: 5-((4-((3-bromo-1H-indazol-1-yl)methyl)-6-fluoro-1H-indol-5-yl)oxy)-2-fluorobenzonitrile

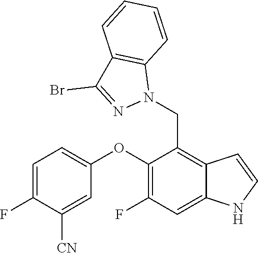 [SMILES: N#Cc1cc(Oc2c(F)cc3[nH]ccc3c2Cn2nc(Br)c3ccccc32)ccc1F]

To a solution of 5-((4-((3-Bromo-1H-indazol-1-yl)methyl)-6-fluoro-1-(phenylsulfonyl)-1H-indol-5-yl)oxy)-2-fluorobenzonitrile (Intermediate 68A, 825 mg, 1.2 mmol) in 5.0 mL of methanol was added potassium carbonate (513 mg, 3.7 mmol). The resulting mixture was stirred at 60° C. overnight and concentrated. The residue was dissolved in ethyl acetate (50 mL), washed with water, brine, dried over sodium sulfate, and concentrated. The crude product was purified by automated flash chromatography (20 g silica gel column, eluting with petroleum ether/ethyl acetate=4/1) to give the title compound (560 mg, 83%) as a white solid. MS (ESI): 479, 481 m/z [M+H]⁺, retention time: 2.18 minutes, purity: 88% (214 nm) (LC-MS method 2).

Intermediate 68A: 5-((4-((3-Bromo-1H-indazol-1-yl)methyl)-6-fluoro-1-(phenylsulfonyl)-1H-indol-5-yl)oxy)-2-fluorobenzonitrile

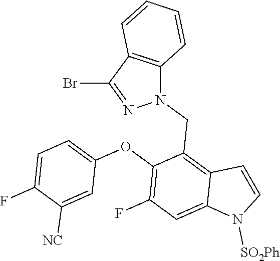

To a stirring solution of compound 5-((4-(bromomethyl)-6-fluoro-1-(phenylsulfonyl)-1H-indol-5-yl)oxy)-2-fluorobenzonitrile (Intermediate 8, 500 mg, 1.0 mmol) in dimethylformamide (10.0 mL) was added 3-bromo-1H-indazole (235 mg, 1.2 mmol) and potassium carbonate (274 mg, 2.0 mmol). The mixture was stirred at 50° C. overnight and concentrated. The residue was re-dissolved in ethyl acetate (50 mL) and washed with water, brine, dried over sodium sulfate, and concentrated. The crude product was purified by automated flash chromatography (20 g silica gel column, eluting with petroleum ether/ethyl acetate=4/1) to give the title compound (340 mg, 49%) as a white solid. MS (ESI): 619,621 m/z [M+H]⁺, retention time: 2.19 minutes, purity: 87% (254 nm) (LC-MS method 2).

Intermediate 69A: 3-(3-Bromophenyl)-6-((1-hydroxypropan-2-yl)oxy)-3-methylhexan-2-ol

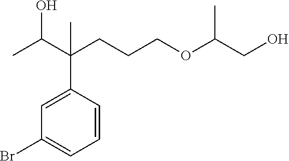 [SMILES: CC(CO)OCCCC(C)(c1cccc(Br)c1)C(C)O]

To a stirred and cooled (−78° C.) solution of 3-(3-bromophenyl)-6-(but-3-en-2-yloxy)-3-methylhexan-2-one (Intermediate 33B-1, 2.0 g, 5.92 mmol) in dichloromethane (30 mL) was bubbled ozone for 0.5 hours until a light blue color was observed. The reaction was stirred at −78° C. for 10 minutes, bubbled with nitrogen for 5 minutes, and then added methanol (3 mL). The mixture was treated with sodium borohydride (0.44 g, 11.84 mmol), warmed to room temperature, and stirred overnight. The reaction was quenched with saturated ammonium chloride (10 mL). The solution was partitioned between (30 mL) and dichloromethane (50 mL). The separated organic phase, combined with two additional dichloromethane extracts (2×50 mL), was washed with brine (2×50 mL), dried over sodium sulfate, and concentrated. The residue was purified by automated flash chromatography (40 g silica gel column, eluting with 0-60% ethyl acetate in petroleum ether) to give the title compound (2.0 g, crude) as an oil. MS (ESI): 367, 369 m/z [M+Na]⁺, retention time: 1.23 minutes, purity: 49% (214 nm) (LC-MS method 2).

The following intermediates were prepared based on the procedures described for Intermediate 69A.

| Inter. | | | |
|---|---|---|---|
| No. | Structure | Name | MS m/z [M + H]⁺ |
| 69A-1 | 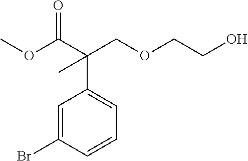 [SMILES: COC(=O)C(C)(COCCO)c1cccc(Br)c1] | methyl 2-(3- bromophenyl)-3-(2- hydroxyethoxy)-2- methylpropanoate | MS: 317, 319 m/z [M + H]+; ¹H NMR (400 MHz, CDCl₃) δ 7.46 (s, 1H), 7.41 (d, J = 7.4 Hz, 1H), 7.25-7.17 (m, 2H), 4.00 (d, J = 8.8 Hz, 1H), 3.72-3.68 (m, 4H), 3.65 (d, J = 8.8 Hz, 1H), 3.58 (dd, J = 8.8 Hz & 4.8 Hz, 2H), 1.71 (s, 1H), 1.65 (s, 3H) ppm. |
| 69A-2 | 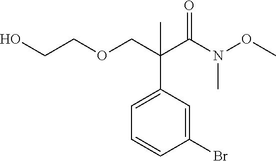 [SMILES: CON(C)C(=O)C(C)(COCCO)c1cccc(Br)c1] | 2-(3-bromophenyl)-3- (2-hydroxyethoxy)-N- methoxy-N,2- dimethylpropanamide | 346, 348 |

Intermediate 69B: 2-((4-(3-bromophenyl)-5-hydroxy-4-methylhexyl)oxy)propanoic acid

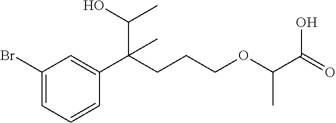 [SMILES: CC(OCCCC(C)(c1cccc(Br)c1)C(C)O)C(=O)O]

To a stirred solution of 3-(3-bromophenyl)-6-((1-hydroxypropan-2-yl)oxy)-3-methylhexan-2-ol (Intermediate 69A, 2.0 g, 5.81 mmol) and (2,2,6,6-Tetramethylpiperidin-1-yl)oxyl (TEMPO, 453 mg, 2.91 mmol) in t-butanol (10 mL) and buffer (pH=4.3 mL) was added sodium chlorite (2.88 g, 32.0 mmol) and a solution of sodium hypochlorite (2.15 mL, 2.91 mmol). The reaction was stirred at room temperature overnight. The mixture was partitioned between water (50 mL) and dichloromethane (50 mL). The separated organic layer, combined with two additional dichloromethane extracts (2×50 mL), was washed with saturated sodium sulfite, saturated sodium bicarbonate, dried over sodium sulfate, and concentrated. The residue was purified by flash chromatography (40 g silica gel column, eluting with 0-60% ethyl acetate in petroleum ether) to give the title compound (1.0 g, 47%) as a white solid. MS (ESI): 381, 383 m/z [M+Na]⁺, retention time: 2.01 minutes, purity: 87% (214 nm) (LC-MS method 2).

Intermediate 69C: Methyl 2-((4-(3-bromophenyl)-5-hydroxy-4-methylhexyl)oxy)propanoate

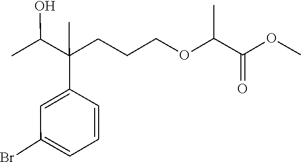 [SMILES: COC(=O)C(C)OCCCC(C)(c1cccc(Br)c1)C(C)O]

To a stirring solution of 2-((4-(3-bromophenyl)-5-hydroxy-4-methylhexyl)oxy) propanoic acid (Intermediate 69B, 1.0 g, 2.79 mmol) in methanol (20 mL) was added concentrated sulfuric acid (0.5 mL). The mixture was stirred at 65° C. overnight and concentrated. The residue was partitioned between ice-water (20 ml) and ethyl acetate (20 mL). The separated organic layer, combined with two additional ethyl acetate extracts (2×20 mL), was washed with brine, dried over sodium sulfate, and concentrated to afford the crude title compound (1.0 g, crude), which was used for the next step without further purification. MS (ESI): 373, 375 m/z [M+H]⁺, retention time: 1.77 minutes, purity: 49% (214 nm) (LC-MS method 2).

The following intermediate was prepared based on the procedures described for Intermediate 69C.

| Inter. | | | MS m/z |
|---|---|---|---|
| No. | Structure | Name | [M + H]⁺ |
| 69C-1 | 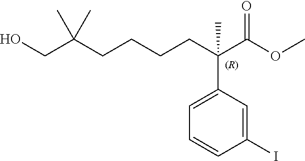 | methyl (R)-8-hydroxy-2-(3-iodophenyl)-2,7,7- trimethyloctanoate | 441 |

Intermediate 69D: Methyl 2-((4-(3-bromophenyl)-4-methyl-5-oxohexyl)oxy)propanoate

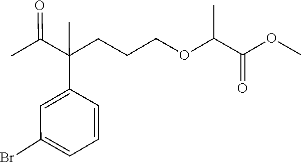 [SMILES: COC(=O)C(C)OCCCC(C)(C(C)=O)c1cccc(Br)c1]

To a stirred solution of methyl 2-((4-(3-bromophenyl)-5-hydroxy-4-methylhexyl)oxy)propanoate (Intermediate 69C, 1.3 g, 2.69 mmol) in dimethyl sulfoxide (5 mL) was added 2-iodoxybenzoic acid (33%, 1.74 g, 3.07 mmol). The reaction was stirred at room temperature overnight, then diluted with water, and extracted with ethyl acetate (3×50 mL). The combined organic extracts were dried over sodium sulfate and concentrated. The residue was purified by flash chromatography (20 g silica gel column, eluting with 0-30% ethyl acetate in petroleum ether) to afford the title compound (1.05 g, 78%). MS (ESI): 393, 395 m/z [M+Na]⁺, retention time: 1.77 minutes, purity: 49% (214 nm) (LC-MS method 2).

Intermediate 70: Methyl 2-((4-(3-bromophenyl)-4-methyl-5-oxohexyl)oxy)-2-methylpropanoate

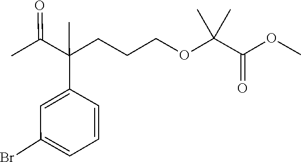 [SMILES: COC(=O)C(C)(C)OCCCC(C)(C(C)=O)c1cccc(Br)c1]

To a stirred and cooled (0° C.) solution of 2-((4-(3-bromophenyl)-4-methyl-5-oxohexyl)oxy)-2-methylpropanoic acid (Intermediate 70B, 3 g, 0.00808 mol) in dichloromethane (50 mL) was added a few drops of dimethylformamide and oxalyl chloride (3.08 g, 0.0242 mol). The reaction was stirred for 2 hours at room temperature, then treated with methanol (50 mL) and stirred for 1 hour. The mixture was concentrated. The residue was purified by silica gel column chromatography (eluting with petroleum ether/ethyl acetate=4:1) to give the title compound (2.9 g, 93%) as a yellow oil. MS (ESI): 407, 409 m/z [M+Na]⁺, retention time: 1.55 minutes, purity: >99% (214 nm) (LC-MS method 9).

Intermediate 70A: 2-((4-(3-Bromophenyl)-4-methyl-5-oxohexyl)oxy)-2-methylpropanal

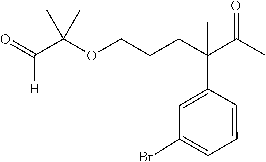 [SMILES: CC(=O)C(C)(CCCOC(C)(C)C=O)c1cccc(Br)c1]

To a stirred and cooled (−78° C.) solution of 3-(3-bromophenyl)-3-methyl-6-((2-methylbut-3-en-2-yl)oxy)hexan-2-one (Intermediate 33B-1, 5.0 g, 0.0142 mol) in dichloromethane (150 mL) was bubbled ozone through for 10 minutes, then treated with dimethyl sulfide (4.40 g, 0.0708 mol). The mixture was allowed to warm to room temperature and stirred for 16 hours and concentrated to give the title compound (5 g, crude) as a yellow oil. MS (ESI): 377, 379 m/z [M+Na]⁺, retention time: 2.17 minutes, purity: 31% (214 nm) (LC-MS method 9).

Intermediate 70B: 2-((4-(3-Bromophenyl)-4-methyl-5-oxohexyl)oxy)-2-methylpropanoic acid

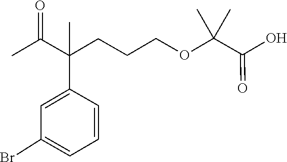 [SMILES: CC(=O)C(C)(CCCOC(C)(C)C(=O)O)c1cccc(Br)c1]

To a stirred solution of 2-((4-(3-bromophenyl)-4-methyl-5-oxohexyl)oxy)-2-methylpropanal (Intermediate 70A, 5.0 g, 0.0141 mol) in t-butanol¶tetrahydrofuran¶water (25 mL/25 mL/25 mL) was added sodium dihydrogen phosphate (5.07 g, 0.0422 mol), sodium chlorite (3.82 g, 0.0422 mol). The mixture was stirred at room temperature for two hours, then partitioned between water (50 mL) and ethyl acetate (50 mL). The separated organic layer, combined with two additional ethyl acetate extracts (2×50 mL), was dried over sodium sulfate and concentrated. The crude was purified by Prep-HPLC (0-90% acetonitrile in water with 0.1 M ammonium bicarbonate modifier) to give the title compound (3 g, 57%) as a yellow oil. MS (ESI): 393, 395 m/z [M+Na]⁺, retention time: 1.81 minutes, purity: >99% (214 nm) (LC-MS method 9).

Intermediate 71: Ethyl 3-(3-(2-methyl-3-oxo-1-(2-(prop-2-yn-1-yloxy)ethoxy)butan-2-yl)phenyl)propanoate

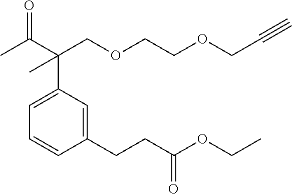 [SMILES: C#CCOCCOCC(C)(C(C)=O)c1cccc(CCC(=O)OCC)c1]

To a stirred and cooled (0° C.) solution of ethyl 3-(3-(1-(2-hydroxyethoxy)-2-methyl-3-oxobutan-2-yl)phenyl)propanoate (Intermediate 26-2, 800 mg, 2.48 mmol) in tetrahydrofuran (20 mL) was added sodium hydride (148 mg, 3.73 mmol). The mixture was stirred at 0° C. for 30 minutes, then treated with 3-bromoprop-1-yne (440 mg, 3.73 mmol) dropwise at this temperature. The reaction was stirred at 0° C. for 1 hours, then warmed to room temperature and stirred overnight. The reaction mixture was quenched with saturated ammonium chloride (50 mL) and extracted with ethyl acetate (3×50 mL). The combined organic layers were washed with brine, dried over sodium sulfate, and concentrated. The residue was purified by silica gel chromatography (eluting with petroleum ether/ethyl acetate=4/1-3/1) to afford the title compound (340 mg, 38%). MS (ESI): 361 m/z [M+H]⁺, retention time: 2.13 minutes, purity: >99% (254 nm) (LC-MS method 2).

Intermediate 72: 2-(3-Bromophenyl)-2,7-dimethyl-7-(3-methyl-2-oxooxazolidin-5-yl)octanoic acid

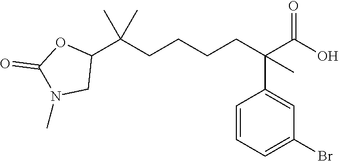 [SMILES: CN1CC(C(C)(C)CCCCC(C)(C(=O)O)c2cccc(Br)c2)OC1=O]

To a stirred and cooled (0° C.) solution of methyl 9-(((benzyloxy)carbonyl)amino)-2-(3-bromophenyl)-8-hydroxy-2,7,7-trimethylnonanoate (Intermediate 49B-1, 6.0 g, 10.7 mmol) in 80 mL of tetrahydrofuran was added sodium hydride (2.13 g, 53.3 mmol). The mixture was stirred at this temperature for 1 hour, then treated with a solution of iodomethane (2.27 g, 16.0 mmol) in 10 mL of dimethylformamide dropwise. The mixture was then slowly allowed to warm to room temperature and stirred overnight, quenched with saturated ammonium chloride, and extracted with ethyl acetate. The combined organic layers were dried over anhydrous sodium sulfate and concentrated. The residue was chromatographed over 0-20% ethyl acetate in petroleum ether to give the title compound (6.2 g, 73%) as a colorless oil. MS (ESI): 426,428 m/z [M+H]⁺, retention time: 2.08 minutes, purity: 80% (254 nm) (LC-MS method 2).

Intermediate 73: 4-Allyl-6-fluoro-5-(4-fluoro-3-(1H-pyrazol-3-yl)phenoxy)-1-tosyl-1H-indole

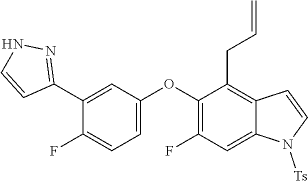 [SMILES: C=CCc1c(Oc2ccc(F)c(-c3cc[nH]n3)c2)c(F)cc2c1ccn2S(=O)(=O)c1ccc(C)cc1]

To a stirred solution of (E)-1-(5-((4-allyl-6-fluoro-1-tosyl-1H-indol-5-yl)oxy)-2-fluorophenyl)-3-(dimethylamino)prop-2-en-1-one (Intermediate 73C, 4.0 g, crude) in ethanol (100 mL) was added hydrazine hydrate (2.2 g, 35 mmol). The reaction was stirred at 50° C. for 2 hours and concentrated. The residue was purified by silica gel column chromatography (petroleum ether: ethyl acetate=1:2) to give the title compound (2.3 g, 66%) as a yellow oil. MS (ESI): 506 m/z [M+H]⁺, retention time: 2.27 minutes, purity: 93% (254 nm) (LC-MS method 5).

The following intermediate was prepared based on the procedures described for Intermediate 73.

| Inter. | | | MS m/z |
|---|---|---|---|
| No. | Structure | Name | [M + H]⁺ |
| 73-1 | 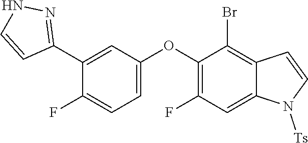 [SMILES: Cc1ccc(S(=O)(=O)n2ccc3c(Br)c(Oc4ccc(F)c(-c5cc[nH]n5)c4)c(F)cc32)cc1] | 4-bromo-6-fluoro-5-(4-fluoro-3-(1H-pyrazol-3- yl)phenoxy)-1-tosyl-1H-indole | 544, 546 |
| 73-2 | 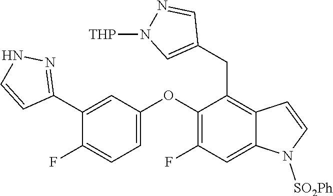 | 6-fluoro-5-(4-fluoro-3-(1H-pyrazol-3-yl)phenoxy)-1- (phenylsulfonyl)-4-((1-(tetrahydro-2H-pyran-2-yl)-1H- pyrazol-4-yl)methyl)-1H-indole | 616 |
| 73-3 | 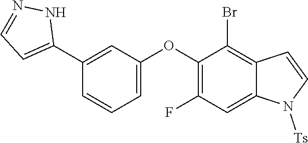 [SMILES: Cc1ccc(S(=O)(=O)n2ccc3c(Br)c(Oc4cccc(-c5ccn[nH]5)c4)c(F)cc32)cc1] | 5-(3-(1H-pyrazol-5-yl)phenoxy)-4-bromo-6-fluoro-1- tosyl-1H-indole | 526, 528 |
| 73-4 | 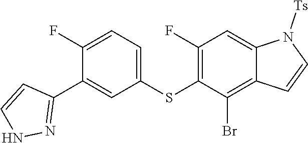 [SMILES: Cc1ccc(S(=O)(=O)n2ccc3c(Br)c(Sc4ccc(F)c(-c5cc[nH]n5)c4)c(F)cc32)cc1] | 4-bromo-6-fluoro-5-((4-fluoro-3-(1H-pyrazol-3- yl)phenyl)thio)-1-tosyl-1H-indole | 560, 562 |
| 73-5 | 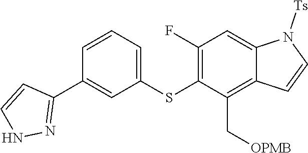 | 5-((3-(1H-pyrazol-3-yl)phenyl)thio)-6-fluoro-4-(((4- methoxybenzyl)oxy)methyl)-1-tosyl-1H-indole | 614 |
| 73-6 | 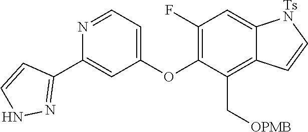 | 5-((2-(1H-pyrazol-3-yl)pyridin-4-yl)oxy)-6-fluoro-4- (((4-methoxybenzyl)oxy)methyl)-1-tosyl-1H-indole | 599 |

Intermediate 73A: 1-(5-((4-Allyl-6-fluoro-1H-indol-5-yl)oxy)-2-fluorophenyl)ethan-1-one

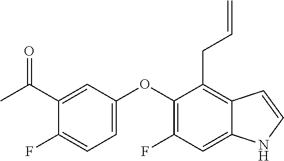 [SMILES: C=CCc1c(Oc2ccc(F)c(C(C)=O)c2)c(F)cc2[nH]ccc12]

To a stirred and degassed solution of 5-((4-allyl-6-fluoro-1H-indol-5-yl)oxy)-2-fluorobenzonitrile (5 g, 16 mmol) in toluene (420 mL) and tetrahydrofuran (80 mL) was added methyl-magnesium bromide solution (3 M in tetrahydrofuran, 16 mL, 48 mmol) at room temperature. The reaction was stirred for 30 minutes, then heated to reflux for 16 hours, and concentrated. The jelly like residue was redissolved in ethyl acetate (100 mL), washed with water, brine, dried over sodium sulfate, and concentrated. The residue was purified by silica gel column chromatography (petroleum ether: ethyl acetate=3:1) to give the title compound (3.8 g, 72%) as brown oil. MS (ESI): 328 m/z [M+H]⁺, retention time: 2.08 minutes, purity: 80% (254 nm) (LC-MS method 5).

The following intermediate was prepared based on the procedures described for Intermediate 73A.

| Inter. | | | MS m/z |
|---|---|---|---|
| No. | Structure | Name | [M + H]⁺ |
| 73A-1 | 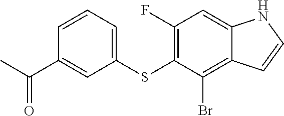 [SMILES: CC(=O)c1cccc(Sc2c(F)cc3[nH]ccc3c2Br)c1] | 1-(3-((4-bromo-6-fluoro-1H-indol-5- yl)thio)phenyl)ethan-1-one | 364, 366 |
| 73A-2 | 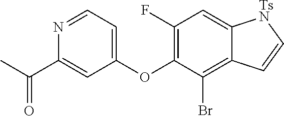 [SMILES: CC(=O)c1cc(Oc2c(F)cc3c(ccn3S(=O)(=O)c3ccc(C)cc3)c2Br)ccn1] | 1-(4-((4-bromo-6-fluoro-1-tosyl-1H-indol-5- yl)oxy)pyridin-2-yl)ethan-1-one | 503, 505 |

Intermediate 73B: 1-(5-((4-Allyl-6-fluoro-1-tosyl-1H-indol-5-yl)oxy)-2-fluorophenyl)ethan-1-one

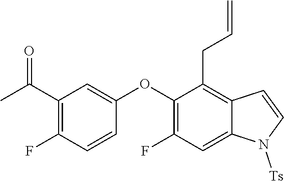 [SMILES: C=CCc1c(Oc2ccc(F)c(C(C)=O)c2)c(F)cc2c1ccn2S(=O)(=O)c1ccc(C)cc1]

To a stirred and cooled (0° C.) solution of 1-(5-((4-allyl-6-fluoro-1H-indol-5-yl)oxy)-2-fluorophenyl)ethan-1-one (Intermediate 73A, 3.8 g, 11.6 mmol) in tetrahydrofuran (150 mL) was added sodium hydride (0.7 g, 17.5 mmol, 60% in mineral oil). The mixture was stirred for 30 minutes at this temperature, then treated with tosyl chloride (4 g, 21 mmol). The reaction was allowed to warm to room temperature and stirred for 2 hours. The solution was partitioned between ethyl acetate (100 mL) and water (80 mL). The separated organic layer was washed with brine, dried over sodium sulfate, and concentrated. The residue was purified by silica gel column chromatography (petroleum ether: ethyl acetate=3:1) to give the title compound (3.3 g, 60%) as a white solid. MS (ESI): 504 m/z [M+Na]⁺, retention time: 2.82 minutes, purity: 99% (254 nm) (LC-MS method 5).

The following intermediate was prepared based on the procedures described for Intermediate 73B.

| Inter. | | | |
|---|---|---|---|
| No. | Structure | Name | MS m/z [M + H]⁺ |
| 73B-1 | 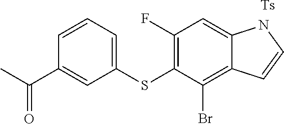 [SMILES: CC(=O)c1cccc(Sc2c(F)cc3[nH]ccc3c2Br)c1 ; "Ts"] | 1-(3-((4-bromo-6-fluoro-1-tosyl-1H-indol-5- yl)thio)phenyl)ethan-1-one | 540, 542 [M + Na]⁺ |

Intermediate 73C: (E)-1-(5-((4-Allyl-6-fluoro-1-tosyl-1H-indol-5-yl)oxy)-2-fluorophenyl)-3-(dimethylamino)prop-2-en-1-one

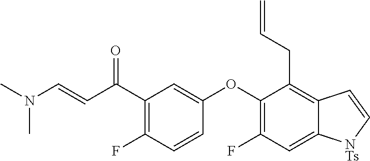 [SMILES: C=CCc1c(Oc2ccc(F)c(C(=O)/C=C/N(C)C)c2)c(F)cc2c1ccn2S(=O)(=O)c1ccc(C)cc1]

To a stirred solution of 1-(5-((4-allyl-6-fluoro-1-tosyl-1H-indol-5-yl)oxy)-2-fluorophenyl)ethan-1-one (Intermediate 73B, 3.3 g, 6.86 mmol) in dimethylformamide (50 mL) was added dimethylformamide dimethylacetal (DMF-DMA, 1 g, 8.2 mmol). The mixture was stirred at 90° C. for 16 hours. The solution was partitioned between water (50 mL) and ethyl acetate (150 mL). The separated organic layer was washed with brine, dried over sodium sulfate and concentrated to give the crude title compound (4.0 g, crude) as a brown oil. MS (ESI): 537 m/z [M+H]⁺, retention time: 2.27 minutes, purity: 95% (254 nm) (LC-MS method 5).

The following intermediate was prepared based on the procedures described for Intermediate 73C.

| Inter. | | | MS m/z |
|---|---|---|---|
| No. | Structure | Name | [M + H]⁺ |
| 73C-1 | 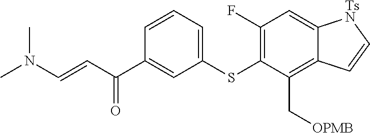 | (E)-3-(dimethylamino)-1-(3-((6-fluoro-4-(((4- methoxybenzyl)oxy)methyl)-1-tosyl-1H-indol-5- yl)thio)phenyl)prop-2-en-1-one | 645 |
| 73C-2 | 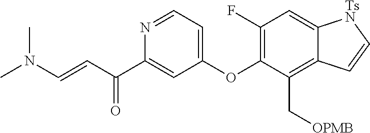 | (E)-3-(dimethylamino)-1-(4-((6-fluoro-4-(((4- methoxybenzyl)oxy)methyl)-1-tosyl-1H-indol-5- yl)oxy)pyridin-2-yl)prop-2-en-1-one | 630 |

Intermediate 74: 1-(2,2-Dimethylbut-3-en-1-yl)-4-(4,4,5,5-tetramethyl-1,3,2-dioxaborolan-2-yl)-1H-pyrazole

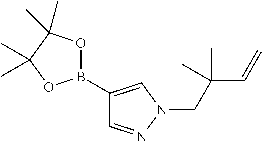 [SMILES: C=CC(C)(C)Cn1cc(B2OC(C)(C)C(C)(C)O2)cn1]

To a stirred and degassed solution of 4-bromo-1-(2,2-dimethylbut-3-en-1-yl)-1H-pyrazole (Intermediate 74D, 1.4 g, 6.11 mmol) in tetrahydrofuran (20 mL) and toluene (20 mL) was added triisopropyl borate (1.38 g, 7.33 mmol). The mixture was cooled to −78° C., then treated with n-butyl lithium (2.5 M in hexanes, 3 mL, 7.33 mmol) over 10 minutes. The mixture was stirred at −78° C. for 15 minutes, added pinacol (1.06 g, 9.2 mmol), and warmed to 25° C. over 40 minutes, and continued stirring for another 80 minutes. The solution was quenched with water (54 mL) over 10 minutes and stirred for an additional 2.5 hours. The formed solid was collected by filtration and dried to afford the title compound (1 g, 59%). MS (ESI): 277 m/z [M+H]⁺, retention time: 1.39 minutes, purity: 86% (214 nm) (LC-MS method 19).

Intermediate 74A: Methyl 3-(4-bromo-1H-pyrazol-1-yl)-2,2-dimethylpropanoate

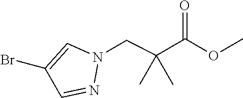

To a stirred solution of methyl 3-bromo-2,2-dimethylpropanoate (12 g, 60 mmol) in dimethylformamide (100 mL) was added cesium carbonate (40 g, 123 mmol) and 4-bromo-1H-pyrazole (9 g, 61.5 mmol). The mixture was stirred at 80° C. for 16 hours, cooled to room temperature and partitioned between water (100 mL) and ethyl acetate (150 mL). The separated organic layer, in combination with two additional ethyl acetate extracts (2×100 mL), was dried over sodium sulfate and concentrated. The residue was purified by flash chromatography (eluting with 0-50% of ethyl acetate in hexane) to give the compound methyl 3-(4-bromo-1H-pyrazol-1-yl)-2,2-dimethylpropanoate (15.6 g, 97%) as a yellow oil. MS (ESI): 261, 263 m/z [M+H]⁺, retention time: 1.20 minutes, purity: 86% (214 nm) (LC-MS method 19).

Intermediate 74B: 3-(4-Bromo-1H-pyrazol-1-yl)-2,2-dimethylpropan-1-ol

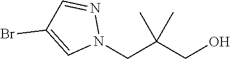

To a stirred and cooled (−78° C.) solution of methyl 3-(4-bromo-1H-pyrazol-1-yl)-2,2-dimethylpropanoate (Intermediate 74A, 15.6 g, 59.7 mmol) in tetrahydrofuran (100 mL) was added lithium aluminum hydride (3.4 g, 89.6 mmol). The reaction was stirred at −78° C. for one hour, then quenched by pouring into a large amount of ice-water and filtered. The mixture was extracted with ethyl acetate (3×150 mL), the combined organic phases were dried over sodium sulfate, and concentrated. The residue was purified by silica gel column chromatography (eluting with petroleum ether/ethyl acetate=8/1) to give the title compound (8.8 g, 63%) as a colorless oil. MS (ESI): 233, 235 m/z [M+H]⁺, retention time: 1.76 minutes, purity: 86% (214 nm) (LC-MS method 19).

Intermediate 74C: 3-(4-Bromo-1H-pyrazol-1-yl)-2,2-dimethylpropanal

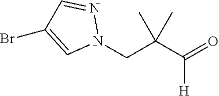

To a stirred and cooled (0° C.) solution of 3-(4-bromo-1H-pyrazol-1-yl)-2,2-dimethylpropan-1-ol (Intermediate 74B, 8.8 g, 37.8 mmol) in dichloromethane (300 mL) was added pyridinium chlorochromate (16.3 g, 75.5 mmol) and 4A molecular sieve (15 g). The reaction was stirred at room temperature for 2 hours, then diluted with petroleum ether (300 mL). The mixture was filtered. The filtrate was concentrated. The residue was purified by silica gel column chromatography (eluting with petroleum ether/ethyl acetate=4/1) to give the title compound (4.7 g, 54%) as a yellow oil.

Intermediate 74D: 4-Bromo-1-(2,2-dimethylbut-3-en-1-yl)-1H-pyrazole

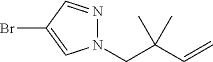

To a stirred solution of methyltriphenylphosphonium bromide (21.8 g, 61 mmol) in tetrahydrofuran (200 mL) was added sodium bis(trimethylsilyl)amide (2 M in tetrahydrofuran, 30.5 mL, 61 mmol). The mixture was stirred for 30 minutes, then treated with 3-(4-bromo-1H-pyrazol-1-yl)-2,2-dimethylpropanal (Intermediate 74C, 4.7 g, 20.3 mmol) dropwise. The reaction was stirred at room temperature for 16 hours, quenched with brine (200 mL), and acidified with 1 N hydrochloric acid to pH ˜5. The mixture was then extracted with ethyl acetate (3×80 mL). The combined organic phase was dried over sodium sulfate and concentrated. The residue was purified by silica gel column chromatography (eluting with petroleum ether/ethyl acetate=8/1) to give the title compound (3.7 g, 79%) as a yellow oil. MS (ESI): 229,231 m/z [M+H]⁺, retention time: 1.34 minutes, purity: 86% (214 nm) (LC-MS method 19).

Intermediate 75: Ethyl 3-(3-(1-hydroxybut-3-en-1-yl)phenyl)propanoate

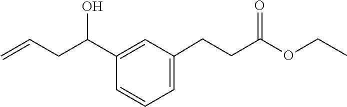 [SMILES: C=CCC(O)c1cccc(CCC(=O)OCC)c1]

To a stirred and cooled (0° C.) suspension of zinc (11 g, 168 mmol) in tetrahydrofuran (130 mL) was added bromine (10 drops). The mixture was stirred for 5 minutes, then treated with allyl bromide (16.2 g, 134 mmol). After 1 hours, ethyl 3-(3-formylphenyl)propanoate (13.8 g, 67.1 mmol) was added dropwise. The mixture was stirred at room temperature for 1 hour, then quenched with brine (100 mL). The solution was acidified with 1 N hydrochloric acid to pH ˜5 and extracted with ethyl acetate (3×80 mL). The combined organic extracts were dried over sodium sulfate and concentrated. The residue was purified by silica gel column chromatography (eluting with petroleum ether/ethyl acetate=8/1) to give the title compound (12.7 g, 76%) as a yellow oil. MS (ESI): 249 m/z [M+H]⁺, retention time: 2.02 minutes, purity: 86% (214 nm) (LC-MS method 19).

Intermediate 76: 4-(((Tert-Butyldiphenylsilyl)oxy)methyl)-6-fluoro-5-(4-fluoro-3-(1H-pyrazol-3-yl)phenoxy)-1-tosyl-1H-indole

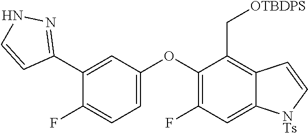

To a stirred and cooled (0° C.) solution of (6-fluoro-5-(4-fluoro-3-(1H-pyrazol-3-yl)phenoxy)-1-tosyl-1H-indol-4-yl)methanol (Intermediate 55-3, 2.6 g, 5.25 mmol) in dimethylformamide (30 mL) was added tert-butyldiphenylsilyl chloride (2.88 g, 10.5 mmol) and imidazole (714 mg, 10.5 mol). The mixture was stirred at room temperature for 60 minutes and quenched with water. The solution was extracted with ethyl acetate (2×50 mL). The combined organic layers were dried over sodium sulfate and concentrated. The residue was purified by silica gel column chromatography (eluting with petroleum ether/ethyl acetate=10/1) to give the title compound (3.7 g, 95%) as a yellow oil. MS (ESI): 734 m/z [M+H]⁺, retention time: 2.93 minutes, purity: 86% (214 nm) (LC-MS method 19).

Intermediate 77: 1-(3-Bromophenyl)-7-((tert-butyldimethylsilyl)oxy)-6,6-dimethylheptan-1-ol

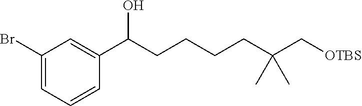 [SMILES: CC(C)(CCCCC(O)c1cccc(Br)c1)CO[Si](C)(C)C(C)(C)C]

To a stirred and cooled (−78° C.) solution of 1-bromo-3-iodo-benzene (14 g, 49.5 mmol) in tetrahydrofuran (150 mL) was added isopropyl magnesium chloride (1.3 M in heptane, 36.7 mL) dropwise. The mixture was stirred for 1 hour, then treated with 7-((tert-butyldimethylsilyl)oxy)-6,6-dimethylheptanal (13.5 g, 49.5 mmol) in 20 mL of tetrahydrofuran dropwise. The mixture was stirred at −78° C. for 3 hours, quenched with 100 mL of aqueous ammonium chloride, and extracted with ethyl acetate (2×150 mL). The combined organic phases were dried over sodium sulfate and concentrated. The residue was purified by chromatography (petroleum ether: ethyl acetate=20:1) to give the title compound (16.4 g, 77%) as yellow oil. ¹H NMR (400 MHz, CDCl₃) δ 7.53 (s, 1H), 7.42 (d, J=7.2 Hz, 1H), 7.28 (s, 1H), 7.25-7.21 (m, 1H), 4.66 (t, J=7.6 Hz, 1H), 3.22 (s, 2H), 1.80-1.67 (m, 2H), 1.29-1.17 (m, 6H), 0.90 (s, 9H), 0.81 (s, 6H), 0.03 (s, 6H) ppm.

The following intermediate was prepared based on the procedures described for Intermediate 77.

| Inter. | | | |
|---|---|---|---|
| No. | Structure | Name | MS m/z [M + H]⁺/¹H NMR |
| 77-1 | 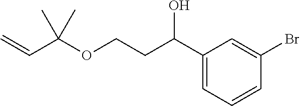 [SMILES: C=CC(C)(C)OCCC(O)c1cccc(Br)c1] | 1-(3-bromophenyl)- 3-((2-methylbut-3- en-2- yl)oxy)propan-1-ol | ¹H NMR (400 MHz, CDCl₃) δ 7.54 (s, 1H), 7.37 (d, J = 8.0 Hz, 1H), 7.28 (d, J = 8 Hz, 1H), 7.19 (t, J = 8.0 Hz,1H), 5.86-5.78 (m, 1H), 5.17-5.12 (m, 2H), 4.89-4.86 (m, 1H), 4.24-4.22 (m, 1H), 3.55-3.50 (m, 2H), 1.94-1.90 (m, 2H), 1.31 (s, 3H), 1.30 (s, 3H) ppm. |

Intermediate 77A: Tert-Butyl((2,2-dimethyloct-7-en-1-yl)oxy)dimethylsilane

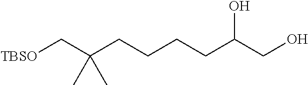 [SMILES: CC(C)(CCCCC(O)CO)CO[Si](C)(C)C(C)(C)C]

To a stirred solution of tert-butyl((2,2-dimethyloct-7-en-1-yl)oxy)dimethylsilane (Intermediate 27C-1, 65 g, 0.24 mol, 1.0 eq.) in acetone (300 mL) and water (60 mL) was added N-methylmorpholine N-oxide (36.6 g, 0.312 mol) and osmium tetroxide (0.61 g, 2.4 mmol). The mixture was stirred at room temperature overnight, then partitioned between water (100 mL) and ethyl acetate (250 mL). The separated organic phase, combined with two additional ethyl acetate extracts, were dried over sodium sulfate and concentrated. The residue was purified by chromatography (eluting with dichloromethane: methanol=20:1) to afford the title compound (63 g, 86%) as yellow oil. ¹H NMR (400 MHz, CDCl₃) δ 3.73-3.69 (m, 2H), 3.48-3.44 (m, 1H), 3.23 (s, 2H), 1.89-1.95 (m, 1H), 1.50-1.40 (m, 2H), 1.38-1.06 (m, 5H), 0.873 (s, 9H), 0.79 (s, 6H), 0.06 (s, 6H) ppm.

Intermediate 77B: 7-((Tert-Butyldimethylsilyl)oxy)-6,6-dimethylheptanal

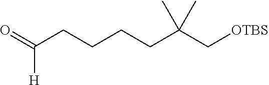 [SMILES: CC(C)(CCCCC=O)CO[Si](C)(C)C(C)(C)C]

To a stirred and cooled (0° C.) solution of tert-butyl((2,2-dimethyloct-7-en-1-yl)oxy)dimethylsilane (Intermediate 77A, 16 g, 52.5 mmol) in acetone (300 mL) and water (100 mL) was added sodium periodate (22.5 g, 0.105 mol) portion wise. The mixture was stirred for two hours, quenched with 50 mL of water and extracted with ethyl acetate (150 mL*2). The combined organic phases were dried over sodium sulfate and concentrated to give the crude title compound (13.5 g, 94%) as yellow oil. It was used for the next step without further purification. ¹H NMR (400 MHz, CDCl₃) δ 9.78 (s, 1H), 3.23 (s, 2H), 2.45 (t, J=7.2 Hz, 2H), 1.64-1.60 (m, 2H), 1.30-1.22 (m, 4H), 0.91 (s, 9H), 0.83 (s, 6H), 0.03 (s, 6H) ppm.

Intermediate 78: Ethyl 2-(6-fluoro-5-(4-fluoro-3-(1H-pyrazol-3-yl)phenoxy)-1-tosyl-1H-indol-4-yl)acetate

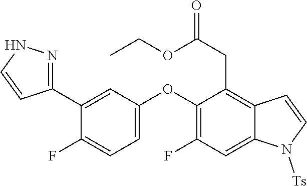 [SMILES: CCOC(=O)Cc1c(Oc2ccc(F)c(-c3cc[nH]n3)c2)c(F)cc2c1ccn2S(=O)(=O)c1ccc(C)cc1]

To a stirred solution of 2-(6-fluoro-5-(4-fluoro-3-(1-(tetrahydro-2H-pyran-2-yl)-1H-pyrazol-3-yl)phenoxy)-1-tosyl-1H-indol-4-yl)acetonitrile (Intermediate 78B, 4.0 g, 6.80 mmol) in ethanol (50 mL) and dichloromethane (30 mL) was added trimethylsilyl chloride (22 g, 204 mmol). The mixture was heated at 60° C. for 2 days then concentrated. The residue was dissolved in 100 mL of ethyl acetate, washed with saturated sodium bicarbonate (30 mL), dried over sodium sulfate and concentrated. The crude title compound (4.0 g, 99%) was obtained as a white solid. MS (ESI): 552 m/z [M+H]⁺, retention time: 1.58 minutes, purity: 87% (214 nm) (LC-MS method 9).

Intermediate 78A: 2-(6-Fluoro-5-(4-fluoro-3-(1-(tetrahydro-2H-pyran-2-y)-1H-pyrazol-3-yl)phenoxy)-1H-indol-4-yl)acetonitrile

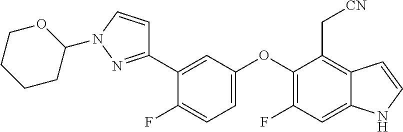 [SMILES: N#CCc1c(Oc2ccc(F)c(-c3ccn(C4CCCCO4)n3)c2)c(F)cc2[nH]ccc12]

To a stirred and degassed solution of 4-bromo-6-fluoro-5-(4-fluoro-3-(1-(tetrahydro-2H-pyran-2-yl)-1H-pyrazol-3-yl)phenoxy)-1H-indole (8.0 g, 16.90 mmol) in dimethyl sulfoxide (30 mL) was added 4-(4,4,5,5-tetramethyl-1,3,2-dioxaborolan-2-yl)isoxazole (4.9 g, 25.34 mmol) and [1,1′-bis(diphenylphosphino)ferrocene]dichloropalladium(II) (Pd(dppf)Cl₂, 1.4 g, 1.69 mmol) and potassium fluoride (16.9 mL, 3 M, 50.7 mmol). The reaction was stirred at 130° C. for 16 hours, cooled to room temperature and quenched with water (50 mL). The mixture was extracted with ethyl acetate (3×50 mL), dried over sodium sulfate. The combined organic phases were washed with brine, dried over sodium sulfate, and concentrated. The residue was purified by silica gel column chromatography (eluting with petroleum ether/ethyl acetate/dichloromethane=2/1/1) to afford the title compound (4.0 g, 54%) as a yellow solid. MS (ESI): 435 m/z [M+H]⁺, retention time: 1.60 minutes, purity: 88% (214 nm) (LC-MS method 9).

Intermediate 78B: 2-(6-Fluoro-5-(4-fluoro-3-(1-(tetrahydro-2H-pyran-2-y)-1H-pyrazol-3-yl)phenoxy)-1-tosyl-1H-indol-4-yl)acetonitrile

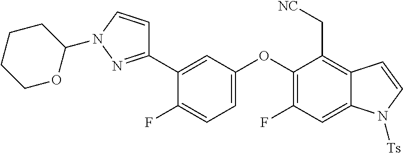 [SMILES: Cc1ccc(S(=O)(=O)n2ccc3c(CC#N)c(Oc4ccc(F)c(-c5ccn(C6CCCCO6)n5)c4)c(F)cc32)cc1]

To a stirred and cooled (0° C.) solution of 2-(6-Fluoro-5-(4-fluoro-3-(1-(tetrahydro-2H-pyran-2-yl)-1H-pyrazol-3-yl)phenoxy)-1H-indol-4-yl)acetonitrile (Intermediate 78A, 4.0 g, 9.22 mmol) in acetonitrile (20 mL) was added 1-(p-tolylsulfonyl)imidazole (4.1 g, 18.44 mmol) and 1,8-diazabicyclo[5.4.0]undec-7-ene (DBU, 0.98 g, 6.45 mmol). The mixture was stirred at room temperature for 16 hours and partitioned between water (50 mL) and ethyl acetate (50 mL). The separated organic layer, combined with two additional ethyl acetate extracts (2×50 mL), was washed with 1 N hydrochloric acid (20 mL), brine, dried over sodium sulfate and concentrated. The residue was purified by column chromatography (petroleum ether/ethyl acetate=3/1) to give the title compound (4.2 g, 77%) as a yellow oil. MS (ESI): 589 m/z [M+H]⁺, retention time: 1.61 minutes, purity: 85% (214 nm) (LC-MS method 9).

Intermediate 79: 4-(2-Methylpent-4-en-2-yl)-1H-pyrazole

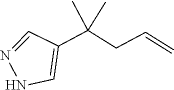

To a stirred solution of 4-(2-methylpent-4-en-2-yl)-1-((2-(trimethylsilyl)ethoxy)methyl)-1H-pyrazole (Intermediate 79F, 2 g, 7.2 mmol) in dichloromethane (10 mL) was added trifluoroacetic acid (10 mL). The mixture was stirred at 40° C. for 16 hours and concentrated. The residue was purified by flash chromatography (24 g silica gel column, eluting with 0-100% ethyl acetate in petroleum ether) to afford the title compound (800 mg, 75%) as oil. MS (ESI): 151 m/z [M+H]⁺, retention time: 1.54 minutes, purity: 90% (214 nm) (LC-MS Method 8).

Intermediate 79A: 2-(1-((2-(Trimethylsilyl)ethoxy)methyl)-1H-pyrazol-4-yl)propan-2-ol

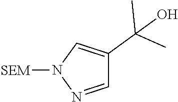 [SMILES: CC(C)(O)c1cnn(COCC[Si](C)(C)C)c1]

To a stirred and cooled (0° C.) solution of ethyl 1-((2-(trimethylsilyl)ethoxy)methyl)-1H-pyrazole-4-carboxylate (30 g, 0.111 mol) in tetrahydrofuran (300 mL) at room temperature was added methylmagnesium bromide (3.0 M in 2-methyltetrahydrofuran, 111 mL, 0.333 mol). The mixture was stirred for 6 hours at room temperature, then quenched with saturated ammonium chloride solution (250 mL) and extracted with ethyl acetate (3×200 mL). The combined organic extracts were washed with saturated lithium chloride, dried over sodium sulfate, and concentrated. The residue was purified by flash chromatography to afford the title compound (26.3 g, 84%). MS (ESI): 257 m/z [M+H]⁺, retention time: 1.88 minutes, purity: 91% (214 nm). (LC-MS Method 02).

Intermediate 79B: 2-Methyl-2-(1-((2-(trimethylsilyl)ethoxy)methyl)-1H-pyrazol-4-yl)propanenitrile

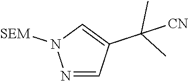 [SMILES: CC(C)(C#N)c1cnn(COCC[Si](C)(C)C)c1]

To a stirred and cooled (0° C.) solution of tris(2,3,4,5,6-pentafluorophenyl)borane (1.27 g, 2.48 mmol) and trimethylsilanecarbonitrile (6.66 mL, 53.2 mmol) in acetonitrile (100 mL) was very slowly added a solution of 2-(1-((2-(trimethylsilyl)ethoxy)methyl)-1H-pyrazol-4-yl)propan-2-ol (Intermediate 79A, 10 g, 35.5 mmol) in acetonitrile (25 mL). The reaction was stirred at room temperature for 1 hour. The mixture was concentrated. The residue was purified by silica gel column chromatography (120 g silica gel column, eluting with 7%-15% ethyl acetate in petroleum ether) to give the title compound (4.3 g, 41%) as a yellow oil. ¹H NMR (400 MHz, CDCl₃) δ 7.59 (s, 1H), 7.55 (s, 1H), 5.42 (s, 2H), 3.59 (t, J=8.0 Hz, 2H), 1.72 (s, 6H), 0.93 (t, J=8.0 Hz, 2H), 0.00 (s, 9H) ppm.

Intermediate 79C: 2-Methyl-2-(1-((2-(trimethylsilyl)ethoxy)methyl)-1H-pyrazol-4-yl)propanal

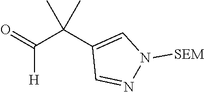 [SMILES: CC(C)(C=O)c1cnn(COCC[Si](C)(C)C)c1]

To a stirred and cooled (−78° C.) solution of 2-methyl-2-(1-((2-(trimethylsilyl)ethoxy)methyl)-1H-pyrazol-4-yl)propanenitrile (Intermediate 79B, 9 g, 34 mmol) in dichloromethane (100 mL) was added diisobutylaluminium hydride (1M in toluene, 51 mL, 51 mmol). The mixture was stirred at −78° C. for 0.5 hours, then allowed to warm to 0° C. over 2 hours and quenched with saturated ammonium chloride (100 mL). The mixture was extracted with dichloromethane (3×100 mL). The combined organic phases were washed with brine, dried over sodium sulfate, and concentrated to give the title compound (7.8 g, 86%). MS (ESI): 269 m/z [M+H]⁺, retention time: 2.15 minutes, purity: 73% (214 nm) (LC-MS Method 02).

Intermediate 79D: (E)-4-(4-Methoxy-2-methylbut-3-en-2-yl)-1-((2-(trimethylsilyl)ethoxy)-methyl)-1H-pyrazole

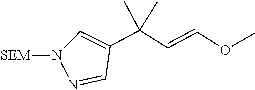

To a stirred and cooled (0° C.) suspension of (methoxymethyl)(triphenyl)phosphonium chloride (28.7 g, 84 mmol) in tetrahydrofuran (200 mL) was added sodium bis(trimethylsilyl)amide (2N in tetrahydrofuran, 42 mL, 84 mmol). The mixture was stirred at 0° C. for 0.5 hours, then treated with a solution of 2-methyl-2-(1-((2-(trimethylsilyl)ethoxy)methyl)-1H-pyrazol-4-yl)propanal (Intermediate 79C, 7.5 g, 28 mmol) in tetrahydrofuran (20 mL). The mixture was then stirred at room temperature for 16 hours, quenched with water (200 mL), and extracted with ethyl acetate (3×100 mL). The combined organic phases were washed with brine, dried over sodium sulfate, and concentrated. The crude material was purified by flash chromatography (120 g silica gel column, eluting with 0-20% ethyl acetate in petroleum ether) to afford the title compound (3.6 g, 44%) as a solid. MS (ESI): 297 m/z [M+H]⁺, retention time: 2.15 minutes, purity: 77% (214 nm) (LC-MS Method 2).

Intermediate 79E: 3-Methyl-3-(1-((2-(trimethylsilyl)ethoxy)methyl)-1H-pyrazol-4-yl)butanal

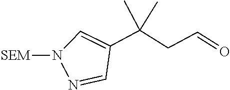 [SMILES: CC(C)(CC=O)c1cnn(COCC[Si](C)(C)C)c1]

To a stirred solution of (E)-4-(4-methoxy-2-methylbut-3-en-2-yl)-1-((2-(trimethylsilyl)ethoxy)-methyl)-1H-pyrazole (Intermediate 79D, 3.6 g, 12.1 mmol) in dichloromethane (60 mL) was added hydrochloric acid in dioxane (4 M, 30 mL). The mixture was stirred at room temperature for four hours, quenched with saturated sodium bicarbonate carefully, and extracted with ethyl acetate (3×100 mL). The combined organic phases were washed with brine, dried over sodium sulfate, and concentrated to give the crude title compound (3 g, 87%), which was used for next step without further purification. MS (ESI): 283 m/z [M+H]⁺, retention time: 1.88 minutes, purity: 80% (214 nm) (LC-MS Method 4).

Intermediate 79F: 4-(2-Methylpent-4-en-2-yl)-1-((2-(trimethylsilyl)ethoxy)methyl)-1H-pyrazole

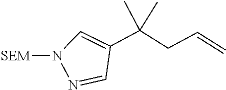

To a stirred and cooled (0° C.) suspension of methyltriphenylphosphonium bromide (11.4 g, 3.2 mmol) in tetrahydrofuran (100 mL) was added sodium bis(trimethylsilyl)amide (2 N in tetrahydrofuran, 16 mL, 32 mmol). The mixture was stirred at 0° C. for 0.5 hours, then treated with a solution of 3-methyl-3-(1-((2-(trimethylsilyl)ethoxy)methyl)-1H-pyrazol-4-yl)butanal (Intermediate 79E, 3 g, 10.6 mmol) in tetrahydrofuran (10 mL). The mixture was stirred at room temperature for 16 hours, quenched with water (100 mL), and extracted with ethyl acetate (3×100 mL). The combined organic phases were washed with brine, dried over sodium sulfate, and concentrated. The residue was purified by flash chromatography (120 g silica gel column, eluting with 0-20% ethyl acetate in petroleum ether) to afford the title compound (2.1 g, 70%) as a solid. MS (ESI): 281 m/z [M+H]⁺, retention time: 2.04 minutes, purity: 95% (214 nm) (LC-MS Method 8).

Intermediate 80: Ethyl 3-(3-(1-hydroxypent-4-en-1-yl)phenyl)propanoate

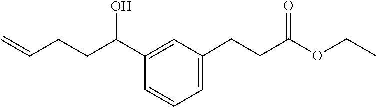 [SMILES: C=CCCC(O)c1cccc(CCC(=O)OCC)c1]

To a stirred and cooled (0° C.) solution of ethyl 3-(3-formylphenyl)propanoate (6 g, 29.1 mmol) in tetrahydrofuran (50 mL) was added a solution of but-3-en-1-ylmagnesium bromide (0.5 M in tetrahydrofuran, 87.3 mL, 43.6 mmol) dropwise over 15 minutes. The reaction mixture stirred for one hour, poured into a saturated ammonium chloride solution (50 mL), and extracted with ethyl acetate (3×60 mL). The combined organic layers were washed with brine, dried over sodium sulfate, and concentrated. The residue was purified by flash column chromatography (eluting with 10% ethyl acetate in petroleum ether) to afford the title compound (4 g, 52%). ¹H NMR (400 MHz, CDCl₃) δ 7.31-7.25 (m, 1H), 7.21-7.15 (m, 2H), 7.10 (d, J=7.6 Hz, 1H), 5.88-5.79 (m, 1H), 5.13-5.03 (m, 1H), 5.01-4.95 (m, 1H), 4.70-4.62 (m, 1H), 4.12 (q, J =7.2 Hz, 2H), 2.98-2.92 (m, 2H), 2.65-2.58 (m, 2H), 2.20-2.05 (m, 2H), 1.89-1.72 (m, 2H), 1.23 (t, J=7.2 Hz, 3H) ppm.

Intermediate 81: 4-(((Tert-Butyldiphenylsilyl)oxy)methyl)-6-fluoro-5-(4-fluoro-3-(1H-pyrazol-3-yl)phenoxy)-1-tosyl-1H-indole

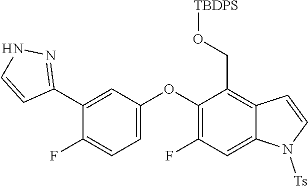

To a stirred and cooled (0° C.) solution of (6-fluoro-5-(4-fluoro-3-(1H-pyrazol-3-yl)phenoxy)-1-tosyl-1H-indol-4-yl)methanol (Intermediate 55-3, 2 g, 4.04 mmol) in dimethylformamide (20 mL) were added tert-butyldiphenylsilyl chloride (2.22 g, 8.07 mmol) and imidazole (550 mg, 8.07 mol). The mixture was stirred at room temperature for 60 minutes. The solution was partitioned between water (50 mL) and ethyl acetate (100 mL). The separated organic layer, combined with two additional ethyl acetate extracts (2×50 mL), was dried over sodium sulfate, and concentrated. The residue was purified by silica gel column chromatography (eluting with 15% ethyl acetate in petroleum ether) to give the title compound (2.5 g, 84%). MS (ESI): 734 m/z [M+H]⁺, retention time: 1.90 minutes, purity: >99% (214 nm) (LC-MS Method 18).

Intermediate 81-1: 2-Fluoro-5-((6-fluoro-1-(phenylsulfonyl)-4-((1-(tetrahydro-2H-pyran-2-yl)-1H-pyrazol-4-yl)methyl)-1H-indol-5-yl)oxy)benzonitrile

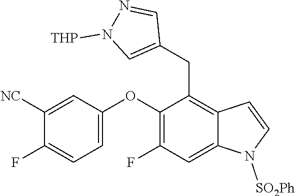

To a stirred and degassed solution of 5-((4-(bromomethyl)-6-fluoro-1-(phenylsulfonyl)-1H-indol-5-yl)oxy)-2-fluorobenzonitrile (Intermediate 8, 29 g, 57.6 mmol) in toluene (420 mL)/ethanol (168 mL) were added 1-(2-methylallyl)-4-(4,4,5,5-tetramethyl-1,3,2-dioxaborolan-2-yl)pyrazole (27.2 g, 98 mmol), sodium bicarbonate (2N, 168 mL, 0.35 mol), palladium tetra(triphenylphosphine) (6.66 g, 5.76 mmol) was stirred at 80° C. for 1 hour. The organic layer was separated, and the aqueous layer was extracted with ethyl acetate (2×200 mL). The combined organic layer was dried over sodium sulfate and concentrated. The residue was purified by flash chromatography (eluting with 0-50% ethyl acetate in petroleum ether) to give the title compound (15 g, 45%) as a solid. MS (ESI): 597 m/z [M+Na]⁺, retention time: 2.18 minutes, purity: 96% (214 nm) (LC-MS Method 2).

Intermediate 82: Methyl 2-fluoro-5-((6-fluoro-4-((4-(2-methylpent-4-en-2-yl)-1H-pyrazol-1-yl)methyl)-1-(phenylsulfonyl)-1H-indol-5-yl)oxy)benzimidothioate hydroiodide

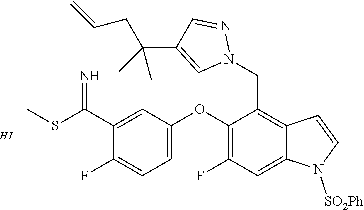

To a stirred solution of 2-fluoro-5-((6-fluoro-4-((4-(2-methylpent-4-en-2-yl)-1H-pyrazol-1-yl)methyl)-1-(phenylsulfonyl)-1H-indol-5-yl)oxy)benzothioamide (Intermediate 82A 2 g, 3.3 mmol) in acetone (10 mL) was added iodomethane (2.34 g, 16.5 mmol). The mixture was stirred at 40° C. for two hours and concentrated. The crude title compound (2 g, 98%) was used for next step without further purification. MS (ESI): 621 m/z [M+H]⁺, retention time: 1.49 minutes, purity: 82% (214 nm) (LC-MS Method 24).

The following intermediates were prepared based on the procedures described for Intermediate 82 and/or for intermediate 82A.

| Inter. | | | MS m/z |
|---|---|---|---|
| No. | Structure | Name | [M + H]⁺ |
| 82-1 | 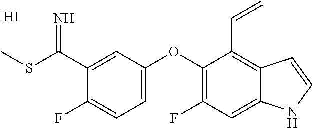 | methyl 2-fluoro-5-((6-fluoro-4-vinyl-1H-indol-5- yl)oxy)benzimidothioate hydroiodide | 345 |
| 82-2 | 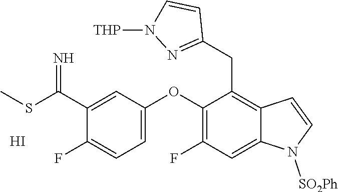 | methyl 2-fluoro-5-((6-fluoro-1-(phenylsulfonyl)-4-((1- (tetrahydro-2H-pyran-2-yl)-1H-pyrazol-3-yl)methyl)-1H- indol-5-yl)oxy)benzimidothioate hydroiodide | 623 |
| 82-3 | 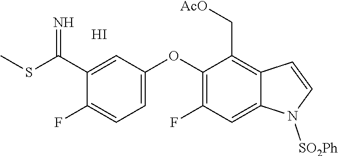 | (6-fluoro-5-(4-fluoro-3- (imino(methylthio)methyl)phenoxy)-1-(phenylsulfonyl)-1H- indol-4-yl)methyl acetate hydroiodide | 531 |
| 82-4 | 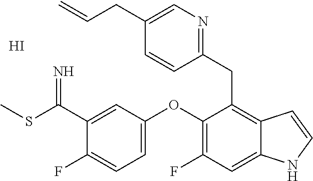 | methyl 5-((4-((5-allylpyridin-2-yl)methyl)-6-fluoro-1H-indol- 5-yl)oxy)-2-fluorobenzimidothioate hydroiodide | 450 |
| 82-5 | 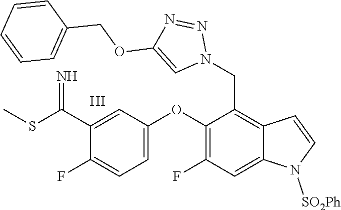 | methyl 5-((4-((4-(benzyloxy)-1H-1,2,3-triazol-1-yl)methyl)- 6-fluoro-1-(phenylsulfonyl)-1H-indol-5-yl)oxy)-2- fluorobenzimidothioate hydroiodide | 646 |
| 82-6 | 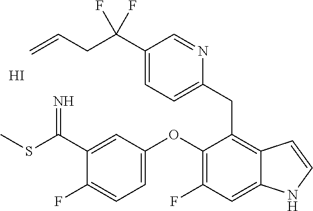 | methyl 5-((4-((5-(1,1-difluorobut-3-en-1-yl)pyridin-2- yl)methyl)-6-fluoro-1H-indol-5-yl)oxy)-2- fluorobenzimidothioate hydroiodide | 500 |
| 82-7 | 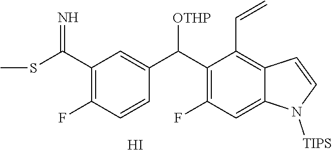 | methyl 2-fluoro-5-((6-fluoro-1-(triisopropylsilyl)-4-vinyl-1H- indol-5-yl)((tetrahydro-2H-pyran-2- yl)oxy)methyl)benzimidothioate hydroiodide | 599 |
| 82-8 | 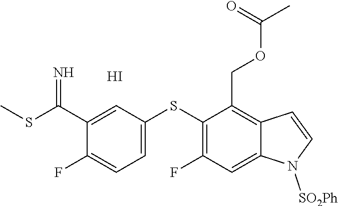 | (6-fluoro-5-((4-fluoro-3- (imino(methylthio)methyl)phenyl)thio)-1-(phenylsulfonyl)- 1H-indol-4-yl)methyl acetate hydroiodide | 547 |
| 82-9 | 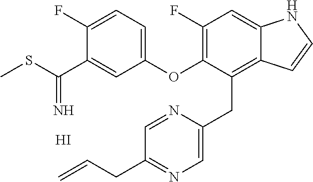 | methyl 5-((4-((5-allylpyrazin-2-yl)methyl)-6-fluoro-1H-indol- 5-yl)oxy)-2-fluorobenzimidothioate hydroiodide | 451 |
| 82-10 | 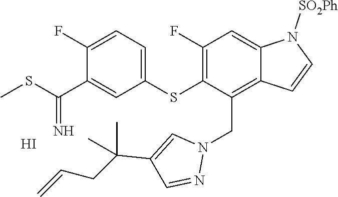 | methyl 2-fluoro-5-((6-fluoro-4-((4-(2-methylpent-4-en-2- yl)-1H-pyrazol-1-yl)methyl)-1-(phenylsulfonyl)-1H-indol-5- yl)thio)benzimidothioate hydroiodide | 637 |
| 82-11 | 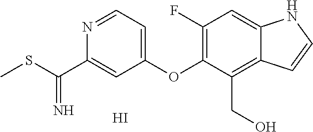 | methyl 4-((6-fluoro-4-(hydroxymethyl)-1H-indol-5- yl)oxy)pyridine-2-carbimidothioate hydroiodide | 332 |
| 82-12 | 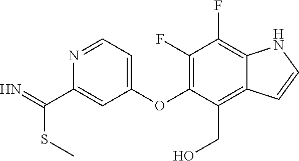 [SMILES: CSC(=N)c1cc(Oc2c(F)c(F)c3[nH]ccc3c2CO)ccn1] | methyl 4-((6,7-difluoro-4-(hydroxymethyl)-1H-indol-5- yl)oxy)pyridine-2-carbimidothioate | 350 [M + H]⁺; RT: 1.13 min. (LC-MS method 2). |

Intermediate 82A: 2-Fluoro-5-((6-fluoro-4-((4-(2-methylpent-4-en-2-yl)-1H-pyrazol-1-yl)methyl)-1-(phenylsulfonyl)-1H-indol-5-yl)oxy)benzothioamide

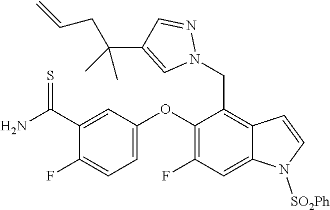

To a stirred solution of 2-fluoro-5-((6-fluoro-4-((4-(2-methylpent-4-en-2-yl)-1H-pyrazol-1-yl)methyl)-1-(phenylsulfonyl)-1H-indol-5-yl)oxy)benzonitrile (Intermediate 85, 2 g, 3.5 mmol) in dimethylformamide (20 mL) was added sodium hydrosulfide (1.17 g, 21 mmol), magnesium chloride (1 g, 10 mmol) and water 0.8 g. The mixture was stirred at room temperature for four hours. The solution was partitioned between water (100 mL) and ethyl acetate (50 mL). The separated organic layer, combined with two additional ethyl acetate extracts (2×50 mL), was washed with brine, dried over sodium sulfate, and concentrated. The residue was purified by flash chromatography to afford the title compound (2 g, 94%). MS (ESI): 607 m/z [M+H]⁺, retention time: 1.58 minutes, purity: 88% (214 nm) (LC-MS Method 24).

Intermediate 83: 2-(3-Bromophenyl)-N′,2-dimethylpent-4-enehydrazide

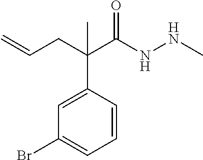 [SMILES: C=CCC(C)(C(=O)NNC)c1cccc(Br)c1]

To a stirred solution of tert-butyl 2-(2-(3-bromophenyl)-2-methylpent-4-enoyl)-1-methylhydrazine-1-carboxylate (13 g, 32.7 mmol) in dichloromethane (40 mL) was added trifluoracetic acid (20 mL). The mixture was stirred at room temperature for four hours and concentrated. The residue was neutralized with saturated sodium bicarbonate (200 mL) and extracted with ethyl acetate (3×100 mL). The combined organic phases were washed with brine (100 mL), dried over sodium sulfate, and concentrated. The residue was purified by automated flash chromatography (120 g silica gel column, eluting with 0-100% ethyl acetate in petroleum ether) to afford the title compound (8 g, 82%) as solid. MS (ESI): 297, 299 m/z [M+H]⁺, retention time: 1.14 minutes, purity: 98% (214 nm) (LC-MS Method 24).

The following intermediates were prepared based on the procedures described for Intermediate 83 and/or for Intermediate 83A.

| Inter. | | | MS m/z |
|---|---|---|---|
| No. | Structure | Name | [M + H]⁺ |
| 83-1 | 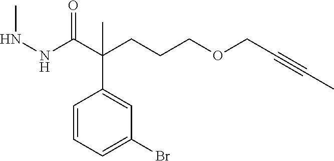 [SMILES: CC#CCOCCCC(C)(C(=O)NNC)c1cccc(Br)c1] | 2-(3-bromophenyl)-5-(but-2-yn-1-yloxy)-N′,2- dimethylpentanehydrazide | 367, 369 |
| 83-2 | 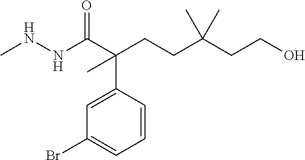 [SMILES: CNNC(=O)C(C)(CCC(C)(C)CCO)c1cccc(Br)c1] | 2-(3-bromophenyl)-7-hydroxy-N′,2,5,5- tetramethylheptanehydrazide | 371, 373 |
| 83-3 | 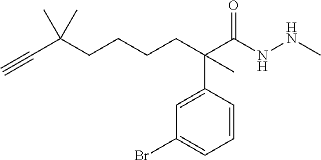 [SMILES: C#CC(C)(C)CCCCC(C)(C(=O)NNC)c1cccc(Br)c1] | 2-(3-bromophenyl)-N′,2,7,7-tetramethylnon-8- ynehydrazide | 379, 381 |
| 83-4 | 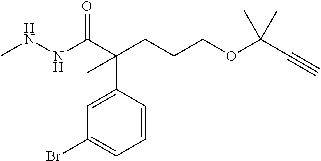 [SMILES: C#CC(C)(C)OCCCC(C)(C(=O)NNC)c1cccc(Br)c1] | 2-(3-bromophenyl)-N′,2-dimethyl-5-((2-methylbut-3-yn- 2-yl)oxy)pentanehydrazide | 381, 383 |
| 83-5 | 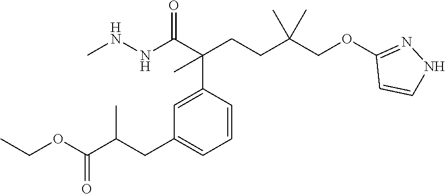 [SMILES: CCOC(=O)C(C)Cc1cccc(C(C)(CCC(C)(C)COc2cc[nH]n2)C(=O)NNC)c1] | ethyl 3-(3-(6-((1H-pyrazol-3-yl)oxy)-2,5,5-trimethyl-1-(2- methylhydrazineyl)-1-oxohexan-2-yl)phenyl)-2- methylpropanoate | 459 |
| 83-6 | 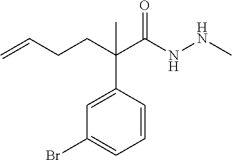 [SMILES: C=CCCC(C)(C(=O)NNC)c1cccc(Br)c1] | 2-(3-bromophenyl)-N′,2-dimethylhex-5-enehydrazide | 311, 313 |
| 83-7 | 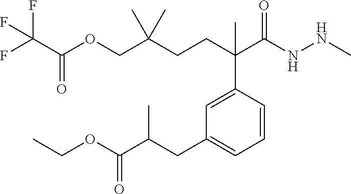 [SMILES: CCOC(=O)C(C)Cc1cccc(C(C)(CCC(C)(C)COC(=O)C(F)(F)F)C(=O)NNC)c1] | ethyl 2-methyl-3-(3-(2,5,5-trimethyl-1-(2- methylhydrazineyl)-1-oxo-6-(2,2,2- trifluoroacetoxy)hexan-2-yl)phenyl)propanoate | 489 |
| 83-8 | 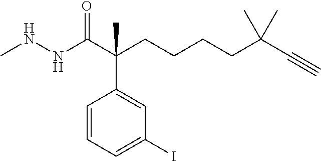 [SMILES: C#CC(C)(C)CCCC[C@@](C)(C(=O)NNC)c1cccc(I)c1] | (R)-2-(3-iodophenyl)-N′,2,7,7-tetramethylnon-8- ynehydrazide | 427 |

Intermediate 83A: Tert-Butyl 2-(2-(3-bromophenyl)-2-methylpent-4-enoyl)-1-methylhydrazine-1-carboxylate

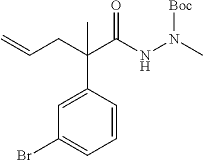 [SMILES: C=CCC(C)(C(=O)NN(C)C(=O)OC(C)(C)C)c1cccc(Br)c1]

To a stirred solution of 2-(3-bromophenyl)-2-methylpent-4-enoic acid (Intermediate 7-30, 11 g, 41 mmol) in acetonitrile (150 mL) were added tert-butyl 1-methylhydrazine-1-carboxylate (6 g, 41 mmol), 1-methylimidazole (11.7 g, 14.3 mmol) and chloro-N,N,N′,N′-tetramethylformamidinium hexafluorophosphate (11.5 g, 41 mmol). The mixture was stirred at room temperature for 2 hours, diluted with 200 mL of ethyl acetate. The solution was washed with 1 N hydrochloric acid, water, brine, dried over sodium sulfate, and concentrated. The residue was purified by silica gel (petroleum ether: ethyl acetate=5:1 to 3:1) to give the title compound (13 g, 80%) as a yellow oil. MS (ESI): 419, 421 m/z [M+Na]⁺, retention time: 1.50 minutes, purity: 92% (214 nm) (LC-MS Method 24).

The following intermediate was prepared based on the procedures described for Intermediate 83A.

| Inter. | | | MS m/z |
|---|---|---|---|
| No. | Structure | Name | [M + H]⁺ |
| 83A-1 | 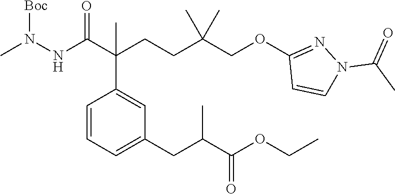 [SMILES: CCOC(=O)C(C)Cc1cccc(C(C)(CCC(C)(C)COc2ccn(C(C)=O)n2)C(=O)NN(C)C(=O)OC(C)(C)C)c1] | tert-butyl 2-(6-((1-acetyl-1H-pyrazol-3-yl)oxy)-2-(3-(3- ethoxy-2-methyl-3-oxopropyl)phenyl)-2,5,5- trimethylhexanoyl)-1-methylhydrazine-1-carboxylate | 623 |

Intermediate 84: 2-(3-Bromophenyl)-5-(but-2-yn-1-yloxy)-2-methylpentanoic acid

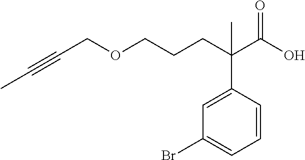 [SMILES: CC#CCOCCCC(C)(C(=O)O)c1cccc(Br)c1]

To a stirred and cooled (−78° C.) solution of 2-(3-bromophenyl)-5-hydroxy-2-methylpentanoic acid (20 g, 69.7 mmol) and 1-bromobut-2-yne (13.9 g, 104 mmol) in tetrahydrofuran (200 mL) was added sodium bis(trimethylsilyl)amide (2 M in tetrahydrofuran, 87 mL, 174 mmol). the mixture was stirred at room temperature for 4 hours. Quenched with saturated ammonium chloride (400 mL) and acidified to pH ˜5 with 1 N hydrochloric acid. The solution was extracted with ethyl acetate (2×200 mL). The combined organic phases were washed with brine, dried over sodium sulfate, and concentrated. The residue was purified by automated silica gel column chromatography (330 g silica gel column, eluting with 0-50% ethyl acetate in petroleum ether) to give the title compound (18 g, 76.2%) as an oil. MS (ESI): 321, 323 m/z [M−H₂O+H]⁺, retention time: 1.86 minutes, purity: 90% (214 nm) (LC-MS Method 5).

The following intermediate was prepared based on the procedures described for Intermediate 83A.

| Inter. | | | MS m/z |
|---|---|---|---|
| No. | Structure | Name | [M + H]⁺ |
| 84-1 | 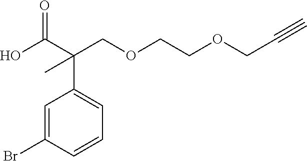 [SMILES: C#CCOCCOCC(C)(C(=O)O)c1cccc(Br)c1] | 2-(3-bromophenyl)-2-methyl-3-(2-(prop-2-yn-1- yloxy)ethoxy)propanoic acid | 363, 365 [M + Na]⁺ |

Intermediate 84A: 2-(3-Bromophenyl)-5-hydroxy-2-methylpentanoic acid

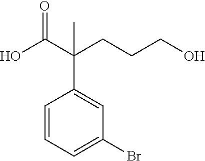 [SMILES: CC(CCCO)(C(=O)O)c1cccc(Br)c1]

To a stirred solution of methyl 2-(3-bromophenyl)-5-((tert-butyldimethylsilyl)oxy)-2-methylpentanoate (Intermediate 7A-26, 37 g, 89.1 mmol) in tetrahydrofuran (100 mL) was added tetrabutylammonium fluoride (1 M in tetrahydrofuran, 223 mL, 223 mmol). The mixture was stirred at 40° C. overnight, quenched with water (400 mL), and extracted with ethyl acetate (2×30 mL). The combined organic phases were washed with brine, dried over sodium sulfate, and concentrated. The residue was redissolved in tetrahydrofuran (200 mL) and water (80 mL), treated with lithium hydroxide monohydrate (7.47 g, 178 mmol). The solution was stirred at 55° C. overnight, diluted with water (200 mL), acidified to pH ˜5 with ethyl acetate (2×200 mL). The combined organic phase was washed with brine, dried over sodium sulfate, and concentrated. The residue was purified by automated silica gel column chromatography (330 g silica gel column, eluting with 0-40% ethyl acetate in petroleum ether) to give the title compound (20 g, yield: 78.2%) as an oil. MS (ESI): 269, 271 m/z [M−H₂O+H]⁺, retention time: 1.54 minutes, purity: 85% (214 nm) (LC-MS Method 5).

Intermediate 85: 2-Fluoro-5-((6-fluoro-4-((4-(2-methylpent-4-en-2-yl)-1H-pyrazol-1-yl)methyl)-1-(phenylsulfonyl)-1H-indol-5-yl)oxy)benzonitrile

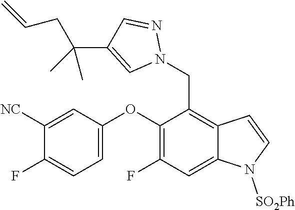

To a stirred solution of 5-((4-(bromomethyl)-6-fluoro-1-(phenylsulfonyl)-1H-indol-5-yl)oxy)-2-fluorobenzonitrile (Intermediate 8, 2.68 g, 4.6 mmol) in dimethylformamide (20 mL) were added 4-(2-methylpent-4-en-2-yl)-1H-pyrazole (Intermediate 79, 0.691 g, 4.6 mmol) and cesium carbonate (3 g, 9.2 mmol). The mixture was stirred at room temperature for 6 hours, then partitioned between water (50 mL) and ethyl acetate (50 mL). The separated organic layer, combined with two additional ethyl acetate extracts (2×50 mL), was washed with 5% lithium chloride solution, dried over sodium sulfate, and concentrated. The residue was purified by automated flash chromatography (80 g silica gel column, eluting with 0-40% ethyl acetate in petroleum ether) to give the title compound (2 g, 76%) as an oil. MS (ESI): 573 m/z [M+H]⁺, retention time: 1.66 minutes, purity: 95% (214 nm) (LC-MS Method 24).

Intermediate 86: 2-Fluoro-5-((6-fluoro-1-(phenylsulfonyl)-4-((1-(tetrahydro-2H-pyran-2-yl)-1H-pyrazol-3-yl)methyl)-1H-indol-5-yl)oxy)benzonitrile

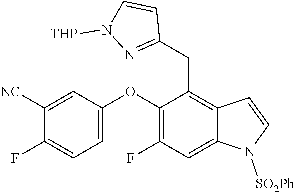

To a stirred and degassed solution of 5-((4-(bromomethyl)-6-fluoro-1-(phenylsulfonyl)-1H-indol-5-yl)oxy)-2-fluorobenzonitrile (Intermediate 8, 7 g, 14 mmol) in 100 mL of toluene/ethanol (5:2) were added 1-(tetrahydro-2H-pyran-2-yl)-5-(4,4,5,5-tetramethyl-1,3,2-dioxaborolan-2-yl)-1H-pyrazole (4.7 g, 17 mmol), tetrakis(triphenylphosphine)palladium(0) (1.6 g, 1.4 mmol), sodium carbonate solution (4.4 g, 42 mmol, 20% in water). The mixture was stirred at 80° C. for 2 hours, cooled to room temperature and quenched with 500 mL of water. The separated organic layer, combined with three additional ethyl acetate extracts (3×100 mL), was washed with saturated lithium chloride, brine, dried over sodium sulfate, and concentrated. The residue was purified by flash chromatography to afford the title compound (5.4 g, 67%). MS (ESI): 597 m/z [M+Na]⁺, retention time: 2.26 minutes, purity: 95% (214 nm) (LC-MS Method 5).

Intermediate 87: (5-(3-Cyano-4-fluorophenoxy)-6-fluoro-1-(phenylsulfonyl)-1H-indol-4-yl)methyl acetate

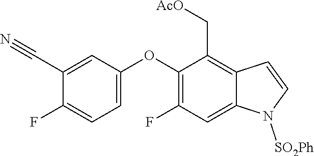

To a stirred solution of 5-((4-(bromomethyl)-6-fluoro-1-(phenylsulfonyl)-1H-indol-5-yl)oxy)-2-fluorobenzonitrile (Intermediate 8, 13 g, 25.8 mmol) in 1,4-dioxane (200 mL) was added potassium acetate (12.7 g, 125 mmol). The mixture was heated to reflux for 16 hours, cooled to room temperature and quenched with saturated sodium bicarbonate (200 mL). The mixture was extracted with ethyl acetate (3×100 mL). The combined organic phases were washed with brine (100 mL), dried over sodium sulfate, and concentrated to give the crude title compound (12.5 g, crude), which was used for the next step without further purification. MS (ESI): 505 m/z [M+Na]⁺, retention time: 1.97 minutes, purity: 90% (214 nm) (LC-MS Method 5).

The following intermediate was prepared based on the procedures described for Intermediate 87.

| Inter. | | | MS m/z |
|---|---|---|---|
| No. | Structure | Name | [M + H]⁺ |
| 87-1 | 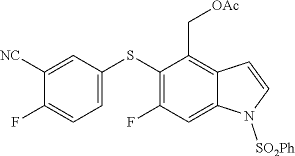 | (5-((3-cyano-4-fluorophenyl)thio)-6-fluoro-1- (phenylsulfonyl)-1H-indol-4-yl)methyl acetate | 499 |

Intermediate 88: 4-((Tert-Butyldimethylsilyl)oxy)-3,3-dimethylbutyl 4-methylbenzenesulfonate

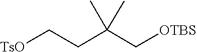 [SMILES: Cc1ccc(S(=O)(=O)OCCC(C)(C)CO[Si](C)(C)C(C)(C)C)cc1]

To a stirred solution of 4-((tert-butyldimethylsilyl)oxy)-3,3-dimethylbutan-1-ol (10.3 g, 44.3 mmol) in pyridine (100 mL) was added toluene-4-sulfonyl chloride (16.9 g, 88.6 mmol). The mixture was stirred at room temperature for four hours, quenched with water (200 mL), and extracted with petroleum ether (2×150 mL). The combined organic phases were washed with brine, dried over sodium sulfate, and concentrated. The residue was purified by silica gel column chromatography (120 g silica gel column, eluting with 0-10% ethyl acetate in petroleum ether) to give the title compound (15 g, 88%) as an oil. MS (ESI): 409 m/z [M+Na]⁺, retention time: 2.42 minutes, purity: 90% (214 nm) (LC-MS Method 2).

Intermediate 88A: (4-(Benzyloxy)-2,2-dimethylbutoxy)(tert-butyl)dimethylsilane

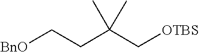 [SMILES: CC(C)(CCOCc1ccccc1)CO[Si](C)(C)C(C)(C)C]

To a stirred solution of 4-(benzyloxy)-2,2-dimethylbutan-1-ol (33 g, 158 mmol) in N,N-dimethylformamide (300 mL) was added imidazole (21.6 g, 317 mmol) and tert-butyldimethylsilyl chloride (25.1 g, 166 mmol). The mixture was stirred at room temperature overnight, quenched with water (200 mL), and extracted with petroleum ether (3×200 mL). The combined organic phases were washed with water, brine, dried over sodium sulfate, and concentrated. The residue was purified by silica gel column chromatography (330 g silica gel column, eluting with 0-10% ethyl acetate in petroleum ether) to give the title compound (48 g, yield: 94%) as an oil. MS (ESI): 323 m/z [M+H]⁺, retention time: 2.89 minutes, purity: 95% (214 nm) (LC-MS Method 2).

Intermediate 88B: 4-((Tert-Butyldimethylsilyl)oxy)-3,3-dimethylbutan-1-ol

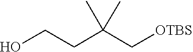

To a stirred solution of (4-(benzyloxy)-2,2-dimethylbutoxy)(tert-butyl)dimethylsilane (Intermediate 88A, 16 g, 49.6 mmol) in ethanol (200 mL) was added palladium hydroxide on active carbon (20%, 3 g). The mixture was stirred at room temperature overnight, then filtered through a pad of Celite. The filtrate was concentrated to give the title compound (11.3 g, 98%) as an oil. MS (ESI): 233 m/z [M+H]⁺, retention time: 2.15 minutes, purity: no UV absorption at 214 nm (LC-MS Method 5).

Intermediate 89: 6-((1-Acetyl-1H-pyrazol-3-yl)oxy)-2-(3-(3-ethoxy-2-methyl-3-oxopropyl)phenyl)-2,5,5-trimethylhexanoic acid

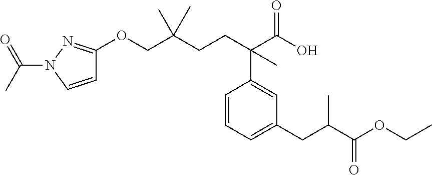 [SMILES: CCOC(=O)C(C)Cc1cccc(C(C)(CCC(C)(C)COc2ccn(C(C)=O)n2)C(=O)O)c1]

To a stirred solution of tert-butyl 6-((1-acetyl-1H-pyrazol-3-yl)oxy)-2-(3-(3-ethoxy-2-methyl-3-oxopropyl)phenyl)-2,5,5-trimethylhexanoate (Intermediate 89A, 5.9 g, 11.2 mmol) in dichloromethane (40 mL) was added trifluoroacetic acid (10 mL). The mixture was stirred at room temperature for four hours and concentrated. The residue was purified by silica gel column chromatography (80 g silica gel column, eluting with 0-50% ethyl acetate in petroleum ether) to give the title compound (5 g, 95%) as an oil. MS (ESI): 473 m/z [M+H]⁺, retention time: 1.98 minutes, purity: 90% (214 nm) (LC-MS Method 2).

Intermediate 89A: Tert-Butyl 6-((1-acetyl-1H-pyrazol-3-yl)oxy)-2-(3-(3-ethoxy-2-methyl-3-oxopropyl)phenyl)-2,5,5-trimethylhexanoate

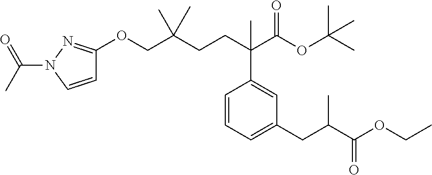 [SMILES: CCOC(=O)C(C)Cc1cccc(C(C)(CCC(C)(C)COc2ccn(C(C)=O)n2)C(=O)OC(C)(C)C)c1]

To a solution of tert-butyl 2-(3-(3-ethoxy-2-methyl-3-oxopropyl)phenyl)-6-hydroxy-2,5,5-trimethylhexanoate (Intermediate 26-3, 6 g, 14.3 mmol) in toluene (50 mL) were added 2-acetyl-1H-pyrazol-5-one (5.4 g, 42.8 mmol), triphenyl phosphine (11.2 g, 42.8 mmol), and diisopropyl azodicarboxylate (8.65 g, 42.8 mmol). The mixture was stirred at 110° C. for 5 hours and concentrated. The residue was purified by silica gel column chromatography (120 g silica gel column, eluting with 0-20% ethyl acetate in petroleum ether) to give the title compound (5.9 g, 78%) as an oil. MS (ESI): 551 m/z [M+Na]⁺, retention time: 2.33 minutes, purity: 95% (214 nm) (LC-MS Method 2).

Intermediate 90: Tert-butyl 2-(6-((1H-pyrazol-3-yl)oxy)-2-(3-(3-ethoxy-2-methyl-3-oxopropyl)phenyl)-2,5,5-trimethylhexanoyl)-1-methylhydrazine-1-carboxylate

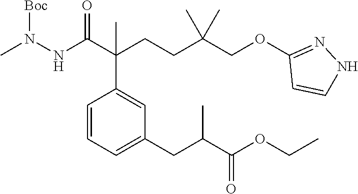 [SMILES: CCOC(=O)C(C)Cc1cccc(C(C)(CCC(C)(C)COc2cc[nH]n2)C(=O)NN(C)C(=O)OC(C)(C)C)c1]

To a stirred solution of tert-butyl 2-(6-((1-acetyl-1H-pyrazol-3-yl)oxy)-2-(3-(3-ethoxy-2-methyl-3-oxopropyl)phenyl)-2,5,5-trimethylhexanoyl)-1-methylhydrazine-1-carboxylate (Intermediate 83A-1, 5 g, 8.32 mmol) in ethanol (50 mL) was added potassium carbonate (2.3 g, 16.6 mmol). The mixture was stirred at room temperature for two hours, then quenched with water (200 mL), and extracted with ethyl acetate (2×150 mL). The combined organic phases were washed with brine, dried over sodium sulfate, and concentrated to give the title compound (4.2 g, 90%) as an oil. MS (ESI): 559 m/z [M+Na]⁺, retention time: 1.91 minutes, purity: 95% (214 nm) (LC-MS Method 2).

Intermediate 91: 2-Fluoro-5-((6-fluoro-4-(4,4,5,5-tetramethyl-1,3,2-dioxaborolan-2-yl)-1H-indol-5-yl)oxy)benzonitrile

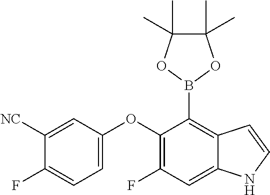 [SMILES: CC1(C)OB(c2c(Oc3ccc(F)c(C#N)c3)c(F)cc3[nH]ccc23)OC1(C)C]

To a stirred solution of 5-((4-bromo-6-fluoro-1H-indol-5-yl)oxy)-2-fluorobenzonitrile (Intermediate 3, 6 g, 0.0172 mol) in dimethyl sulfoxide (30 mL) was added 4,4,5,5-tetramethyl-2-(4,4,5,5-tetramethyl-1,3,2-dioxaborolan-2-yl)-1,3,2-dioxaborolane (6.55 g, 0.0258 mol), 1,1′-bis(diphenylphosphino)ferrocene-palladium(ii)dichloride dichloromethane complex (702 mg, 0.859 mmol), potassium acetate (3.37 g, 0.0344 mol) under argon. The mixture was stirred at 130° C. for 6 hours, cooled to room temperature, and filtered over a pad of silica gel. The filter cake was washed with tetrahydrofuran (60 mL). The filtrate was diluted with ethyl acetate (100 mL), washed with brine, dried over sodium sulfate, and concentrated. The crude was purified by silica gel column chromatography (eluting with petroleum ether: dichloromethane=1:1) to give the title compound (1.68 g, 25%) as a yellow solid. MS (ESI): 397 m/z [M+H]⁺, retention time: 1.94 minutes, purity: 73% (214 nm) (LC-MS Method 26).

Intermediate 92: 5-((4-((5-Allylpyridin-2-yl)methyl)-6-fluoro-1H-indol-5-yl)oxy)-2-fluorobenzonitrile

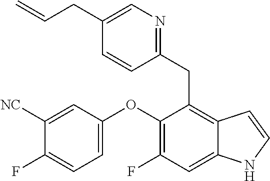 [SMILES: C=CCc1ccc(Cc2c(Oc3ccc(F)c(C#N)c3)c(F)cc3[nH]ccc23)nc1]

To a stirred and degassed solution of 5-((4-((5-chloropyridin-2-yl)methyl)-6-fluoro-1H-indol-5-yl)oxy)-2-fluorobenzonitrile (Intermediate 92A, 1.70 g, 4.30 mmol) in 1,4-dioxane (30 mL) was added allyl(tributyl)stannane (7.11 g, 0.0215 mol), tris(dibenzylideneacetone)dipalladium(0) (123 mg, 0.215 mmol), tri-tert-butylphosphonium tetrafluoroborate (249 mg, 0.859 mmol) and cesium fluoride (1.44 g, 9.45 mmol) under argon. The mixture was stirred at 120° C. overnight, cooled to room temperature, and diluted with 50 mL of water. The solution was extracted with ethyl acetate (3×50 mL). The combined organic phases were washed with brine, dried over sodium sulfate and concentrated. The residue was purified by silica gel column chromatography (eluting with petroleum ether: ethyl acetate=2:1) to give the title compound (1.05 g, 61%) as a yellow solid. MS (ESI): 402 m/z [M+H]⁺, retention time: 1.60 minutes, purity: 96% (214 nm) (LC-MS Method 26).

Intermediate 92A: 5-((4-((5-Chloropyridin-2-yl)methyl)-6-fluoro-1H-indol-5-yl)oxy)-2-fluorobenzonitrile

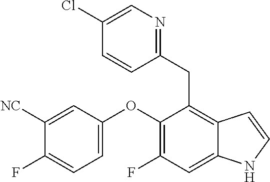 [SMILES: N#Cc1cc(Oc2c(F)cc3[nH]ccc3c2Cc2ccc(Cl)cn2)ccc1F]

To a stirred and degassed solution of 2-fluoro-5-((6-fluoro-4-(4,4,5,5-tetramethyl-1,3,2-dioxaborolan-2-yl)-1H-indol-5-yl)oxy)benzonitrile (Intermediate 91, 2.80 g, 7.07 mmol) in 1,4-dioxane (40 mL) and water (8 mL) was added 5-chloro-2-(chloromethyl)pyridine (1.72 g, 0.0106 mol), 1,1′-bis(diphenylphosphino)ferrocene-palladium(II)dichloride dichloromethane complex (577 mg, 0.707 mmol) and potassium carbonate (1.95 g, 0.0141 mol). The mixture was stirred at 100° C. for 1 hour, cooled to room temperature and quenched with 100 mL of water. The solution was extracted with ethyl acetate (3×100 mL). The combined organic phases were washed with brine, dried over sodium sulfate, and concentrated. The residue was purified by silica gel column chromatography (eluting with petroleum ether: ethyl acetate=3:1) to give the title compound (1.70 g, 61%) as a yellow solid. MS (ESI): 396 m/z [M+H]⁺, retention time: 1.89 minutes, purity: 73% (214 nm) (LC-MS Method 26).

The following intermediate was prepared based on the procedures described for Intermediate 92A.

| Inter. | | | MS m/z |
|---|---|---|---|
| No. | Structure | Name | [M + H]⁺ |
| 92A-1 | 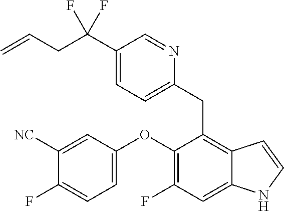 [SMILES: C=CCC(F)(F)c1ccc(Cc2c(Oc3ccc(F)c(C#N)c3)c(F)cc3[nH]ccc23)nc1] | 5-((4-((5-(1,1-difluorobut-3-en-1-yl)pyridin-2-yl)methyl)-6-fluoro- 1H-indol-5-yl)oxy)-2-fluorobenzonitrile | 452 |

Intermediate 93: 5-((4-((4-(Benzyloxy)-1H-1,2,3-triazol-1-yl)methyl)-6-fluoro-1-(phenylsulfonyl)-1H-indol-5-yl)oxy)-2-fluorobenzonitrile

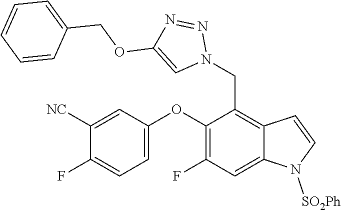

To a stirred and degassed solution of 5-((4-(azidomethyl)-6-fluoro-1-(phenylsulfonyl)-1H-indol-5-yl)oxy)-2-fluorobenzonitrile (Intermediate 9A, 4 g, 8.59 mmol) in dichloromethane (50 mL) was added ethynoxymethylbenzene (2.6 g, crude), tetrakis(acetonitrile)copper(I) tetrafluoroborate (0.54 g, 1.72 mmol) and tris((1-benzyl-1H-1,2,3-triazol-4-yl)methyl)amine (0.912 g, 1.72 mmol). The mixture was stirred at 45° C. overnight. The solvent was removed under vacuum. The residue was purified by silica gel column chromatography (80 g silica gel column, eluting with 0-40% ethyl acetate in petroleum ether) to give the title compound (3.8 g, 74%) as a solid. MS (ESI): 598 m/z [M+H]⁺, retention time: 1.97 minutes, purity: 95% (214 nm) (LC-MS Method 2).

Intermediate 93A: (Z)-(((1,2-Dichlorovinyl)oxy)methyl)benzene

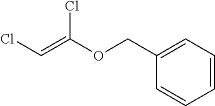 [SMILES: Cl/C=C(\Cl)OCc1ccccc1]

To a suspension of potassium hydride (7.72 g of 30% in mineral oil, theoretically 2.23 g, 55.5 mmol, (mineral oil was removed by triturating with pentane (3×50 mL)) in tetrahydrofuran (30 mL) was added dropwise a solution of phenyl methanol (3 g, 27.7 mmol) in tetrahydrofuran (10 mL) at room temperature. The mixture was stirred at room temperature for 30 minutes, then cooled to −78° C., and treated with a solution of 1,1,2-trichloroethylene (4.01 g, 30.5 mmol) in tetrahydrofuran (10 mL) dropwise over 10 minutes. The reaction mixture was allowed to warm to room temperature and stirred for one additional hour and quenched with water (100 mL). The mixture was extracted with petroleum ether (2×80 mL). The organic phase was washed with brine, dried over sodium sulfate, and concentrated. The residue was purified by automated silica gel column chromatography (80 g silica gel column, eluting with 0-10% ethyl acetate in Petroleum ether) to give the title compound (3.5 g, 62%) as an oil. ¹H NMR (400 MHz, CDCl₃) δ 7.47-7.34 (m, 5H), 5.52 (s, 1H), 5.05 (s, 2H) ppm.

Intermediate 93B: ((Ethynyloxy)methyl)benzene

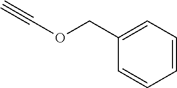

To a stirred and cooled (−78° C.) solution of (Z)-(((1,2-dichlorovinyl)oxy)methyl)benzene (3.6 g, 19.2 mmol) and N,N,N′,N′-tetramethyl-ethylenediamine (4.46 g, 38.4 mmol) in tetrahydrofuran (40 mL) was added n-butyllithium (2.5 M in hexanes, 15.4 mL, 38.4 mmol) dropwise over 10 minutes. The reaction mixture was stirred at −78° C. for 30 minutes and at −40° C. for 30 minutes, then cooled to −78° C. The mixture was treated with 20 mL of 10% ethanol-pentane at −78° C. After 5 minutes, the cold mixture was diluted with pentane (100 mL), washed with water, brine, dried over sodium sulfate, and concentrated to give the crude title compound (2.54 g, crude) as an oil, which was used for next step without further purification.

Intermediate 94: 2-(3-(3-Ethoxy-2-methyl-3-oxopropyl)phenyl)-2,5,5-trimethyl-6-(2,2,2-trifluoroacetoxy)hexanoic acid

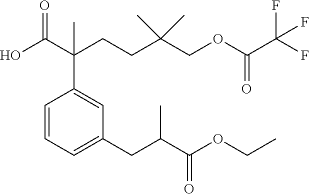 [SMILES: CCOC(=O)C(C)Cc1cccc(C(C)(CCC(C)(C)COC(=O)C(F)(F)F)C(=O)O)c1]

To a stirred solution of tert-butyl 2-(3-(3-ethoxy-2-methyl-3-oxopropyl)phenyl)-6-hydroxy-2,5,5-trimethylhexanoate (Intermediate 26-3, 3.7 g, 8.8 mmol) in dichloromethane (20 mL) was added trifluoroacetic acid (5 mL). The mixture was stirred at room temperature for four hours and concentrated. The residue was purified by silica gel column chromatography (80 g silica gel column, eluting with 0-50% ethyl acetate in petroleum ether) to give the title compound (3.7 g, 91%) as an oil. MS (ESI): 483 m/z [M+Na]⁺, retention time: 2.00 minutes, purity: 90% (214 nm) (LC-MS Method 2).

Intermediate 95: Ethyl 3-(3-(6-hydroxy-2,5,5-trimethyl-1-(2-methylhydrazineyl)-1-oxohexan-2-yl)phenyl)-2-methylpropanoate

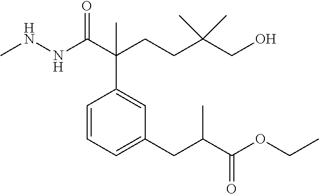 [SMILES: CCOC(=O)C(C)Cc1cccc(C(C)(CCC(C)(C)CO)C(=O)NNC)c1]

To a stirred solution of ethyl 2-methyl-3-[3-[1,4,4-trimethyl-1-(methylaminocarbamoyl)-5-(2,2,2-trifluoroacetyl)oxy-pentyl]phenyl]propanoate (Intermediate 83-7, 3.2 g, 6.55 mmol) in ethanol (30 mL) was added potassium carbonate (1.81 g, 13.1 mmol). The mixture was stirred at room temperature for four hours, quenched with water (100 mL), and extracted with ethyl acetate (2×80 mL). The combined organic phases were washed with brine, dried over sodium sulfate, filtered and concentrated. The residue was purified by silica gel column chromatography (80 g silica gel column, eluting with 0-70% ethyl acetate in petroleum ether) to give the title compound (2.2 g, 85%) as an oil. MS (ESI): 393 m/z [M+H]⁺, retention time: 1.51 minutes, purity: 90% (214 nm) (LC-MS Method 2).

Intermediate 96: 3-((6-Fluoro-4-vinyl-1H-indol-5-yl)oxy)-N-methylbenzimidamide

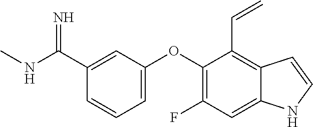 [SMILES: C=Cc1c(Oc2cccc(C(=N)NC)c2)c(F)cc2[nH]ccc12]

To a stirred solution of 3-((6-fluoro-4-vinyl-1H-indol-5-yl)oxy)benzonitrile (4.0 g, 13 mmol) in 250 mL of toluene was added methylamine hydrochloride (19.2 g, 285 mmol) and trimethylaluminum (1 M in hexane, 259 mL). The mixture was stirred at 100° C. for 16 hours, cooled to room temperature, quenched with water (300 mL) and tetrahydrofuran (300 mL). The mixture was filtered. The filtrate was extracted with ethyl acetate (3×100 mL). The combined organic phases were washed with brine, dried over sodium sulfate, and concentrated. The residue was purified by flash chromatography to afford the title compound (3 g, 75%). MS (ESI): 310 m/z [M+H]⁺.

Intermediate 97: Ethyl 3-(3-(6-((1H-pyrazol-3-yl)oxy)-5,5-difluoro-2-methyl-1-(2-methylhydrazineyl)-1-oxohexan-2-yl)phenyl)-2-methylpropanoate

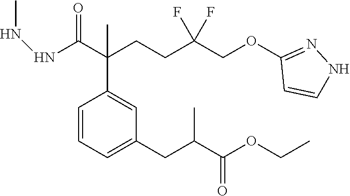 [SMILES: CCOC(=O)C(C)Cc1cccc(C(C)(CCC(F)(F)COc2cc[nH]n2)C(=O)NNC)c1]

To a stirred solution of ethyl 3-(3-(6-((1-acetyl-1H-pyrazol-3-yl)oxy)-5,5-difluoro-2-methyl-1-(2-methylhydrazineyl)-1-oxohexan-2-yl)phenyl)-2-methylpropanoate (Intermediate 97H, 800 mg, 1.57 mmol) in ethanol (20 mL) was added potassium carbonate (435 mg, 3.15 mmol). The reaction was stirred at room temperature for two hours. The mixture was diluted with ethyl acetate (50 mL), washed with water, dried over magnesium sulfate, and concentrated. The residue was purified by flash chromatography (20 g silica gel column, eluting with 0-80% ethyl acetate in petroleum) to give the title compound (320 mg, 44%) as oil. MS (ESI): 467 m/z [M+H]⁺, retention time: 1.53 minutes, purity: 85% (254 nm) (LC-MS Method 5).

The following intermediates were prepared based on the procedures described for Intermediate 97 and/or for Intermediate 97A to 97H.

| Inter. | | | MS m/z |
|---|---|---|---|
| No. | Structure | Name | [M + H]⁺ |
| 97-1 | 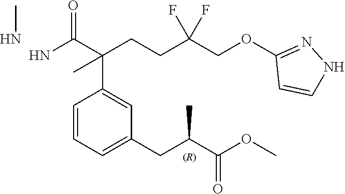 | methyl (2R)-3-(3-(6-((1H-pyrazol-3-yl)oxy)-5,5-difluoro-2- methyl-1-(2-methylhydrazineyl)-1-oxohexan-2-yl)phenyl)-2- methylpropanoate | 453 |
| 97-2 | 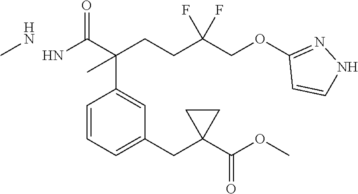 | methyl 1-(3-(6-((1H-pyrazol-3-yl)oxy)-5,5-difluoro-2-methyl-1- (2-methylhydrazineyl)-1-oxohexan-2-yl)benzyl)cyclopropane- 1-carboxylate | 465 |

Intermediate 97A: Methyl 6-(benzyloxy)-2-(3-bromophenyl)-2-methyl-5-oxohexanoate

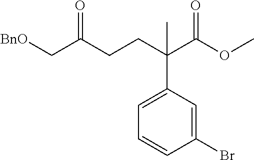 [SMILES: COC(=O)C(C)(CCC(=O)COCc1ccccc1)c1cccc(Br)c1]

To a stirred and cooled (−78° C.) solution of methyl 2-(3-bromophenyl)propanoate (13.8 g, 56.7 mmol) in tetrahydrofuran (250 mL) was added lithium diisopropylamide (2.0 M, 28.4 mL, 56.7 mmol) over 15 minutes. The mixture was stirred for 1 h at −78° C. and treated dropwise with a solution of 1-(benzyloxy)but-3-en-2-one (5 g, 28.4 mmol) in tetrahydrofuran (50 mL). The reaction was warmed to room temperature and stirred for 16 hours, then quenched with water (300 mL), and extracted with ethyl acetate (2×200 mL). The combined organic extracts were washed with brine, dried over anhydrous sodium sulfate, and concentrated. The residue was purified by flash chromatography (120 g silica gel column, eluting with 0-15% ethyl acetate in petroleum ether) to give the title compound (7.2 g, 61%) as oil. MS (ESI): 441, 443 m/z [M+Na]⁺, retention time: 1.97 minutes, purity: 90% (214 nm) (LC-MS Method 2).

The following intermediates were prepared based on the procedures described for Intermediate 97A.

| Inter. | | | MS m/z |
|---|---|---|---|
| No. | Structure | Name | [M + H]⁺ |
| 97A-1 | 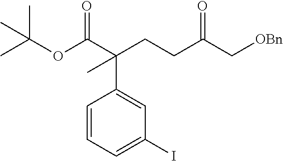 [SMILES: CC(C)(C)OC(=O)C(C)(CCC(=O)COCc1ccccc1)c1cccc(I)c1] | tert-butyl 6-(benzyloxy)-2-(3-iodophenyl)-2-methyl-5- oxohexanoate | 531 [M + H]⁺ |
| 97A-2 | 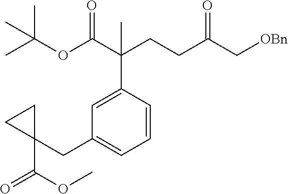 [SMILES: COC(=O)C1(Cc2cccc(C(C)(CCC(=O)COCc3ccccc3)C(=O)OC(C)(C)C)c2)CC1] | methyl 1-(3-(6-(benzyloxy)-1-(tert-butoxy)-2-methyl-1,5- dioxohexan-2-yl)benzyl)cyclopropane-1-carboxylate | 517 [M + H]⁺ |

Intermediate 97B: Methyl 6-(benzyloxy)-2-(3-bromophenyl)-5,5-difluoro-2-methylhexanoate

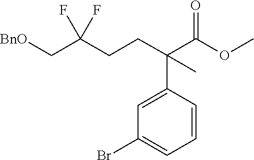 [SMILES: COC(=O)C(C)(CCC(F)(F)COCc1ccccc1)c1cccc(Br)c1]

To a stirred solution of methyl 6-(benzyloxy)-2-(3-bromophenyl)-2-methyl-5-oxohexanoate (Intermediate 97A, 7.2 g, 17.2 mmol) in 1-octyl-3-methylimidazolium hexafluorophosphate (40 mL) was added diethylaminosulfur trifluoride (11.3 mL, 85.9 mmol) at room temperature. The mixture was stirred at 50° C. for four hours, cooled to room temperature and quenched with saturated sodium bicarbonate (200 mL). The solution was extracted with ethyl acetate (200 mL), washed with brine, dried over sodium sulfate, and concentrated. The residue was purified by flash chromatography (80 g silica gel column, eluting with 0-10% ethyl acetate in petroleum ether) to give the title compound (5.1 g, 67.3%) as an oil. MS (ESI): 441, 443 m/z [M+H]⁺, retention time: 2.08 minutes, purity: 90% (254 nm) (LC-MS Method 2).

The following intermediates were prepared based on the procedures described for Intermediate 97B.

| Inter. | | | MS m/z |
|---|---|---|---|
| No. | Structure | Name | [M + H]⁺ |
| 97B-1 | 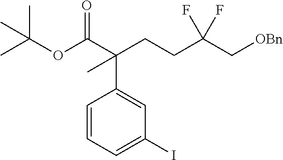 [SMILES: CC(C)(C)OC(=O)C(C)(CCC(F)(F)COCc1ccccc1)c1cccc(I)c1] | tert-butyl 6-(benzyloxy)-5,5-difluoro-2-(3-iodophenyl)-2- methylhexanoate | 553 [M + Na]⁺ |
| 97B-2 | 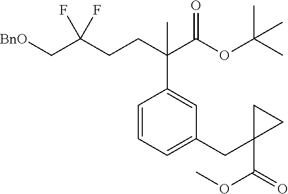 [SMILES: COC(=O)C1(Cc2cccc(C(C)(CCC(F)(F)COCc3ccccc3)C(=O)OC(C)(C)C)c2)CC1] | methyl 1-(3-(6-(benzyloxy)-1-(tert-butoxy)-5,5-difluoro-2- methyl-1-oxohexan-2-yl)benzyl)cyclopropane-1-carboxylate | 539 [M + Na]⁺ |

Intermediate 97C: 6-(Benzyloxy)-2-(3-bromophenyl)-5,5-difluoro-2-methylhexanoic acid

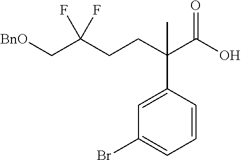 [SMILES: CC(CCC(F)(F)COCc1ccccc1)(C(=O)O)c1cccc(Br)c1]

To a stirred solution of methyl 6-(benzyloxy)-2-(3-bromophenyl)-5,5-difluoro-2-methylhexanoate (Intermediate 97B, 5.1 g, 11.6 mmol) in tetrahydrofuran (120 mL) and methanol (40 mL) was added lithium hydroxide (34.7 mL, 1M in water). The reaction was stirred at 50° C. for 16 hours, cooled to room temperature and acidified with 1 M hydrochloric acid (100 mL). The mixture was extracted with ethyl acetate (100 mL). The combined organic extracts were washed with brine, dried over sodium sulfate, and concentrated to give the crude title compound (4.7 g, 95%) as oil. MS (ESI): 449, 451 m/z [M+Na]⁺, retention time: 1.93 minutes, purity: 90% (254 nm) (LC-MS Method 5).

Intermediate 97D: (E)-6-(Benzyloxy)-2-(3-(3-ethoxy-2-methyl-3-oxoprop-1-en-1-yl)phenyl)-5,5-difluoro-2-methylhexanoic acid

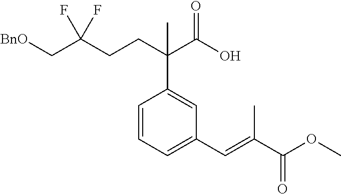 [SMILES: COC(=O)/C(C)=C/c1cccc(C(C)(CCC(F)(F)COCc2ccccc2)C(=O)O)c1]

To a stirred and degassed solution of 6-(benzyloxy)-2-(3-bromophenyl)-5,5-difluoro-2-methylhexanoic acid (Intermediate 97C, 4.7 g, 11.0 mmol) in N,N-dimethylformamide (50 mL) was added ethyl methacrylate (4.11 mL, 33.0 mmol), triethylamine (7.67 mL, 55.0 mmol), tri(o-tolyl)phosphine (1 g, 3.3 mmol), and palladium (II) acetate (247 mg, 1.1 mmol). The reaction was stirred at 120° C. for 16 hours, cooled to room temperature, and acidified with 1 M hydrochloric acid to pH ˜4. The solution was diluted with ethyl acetate (200 mL), washed with water, brine, dried over sodium sulfate, and concentrated. The residue was purified by flash chromatography (80 g silica gel column, eluting with 0-40% ethyl acetate in petroleum ether) to give the title compound (3.1 g, 61%) as an oil. MS (ESI): 483 m/z [M+Na]⁺, retention time: 1.95 minutes, purity: 85% (254 nm) (LC-MS Method 5).

Intermediate 97E: Tert-butyl (E)-2-(6-(benzyloxy)-2-(3-(3-ethoxy-2-methyl-3-oxoprop-1-en-1-yl)phenyl)-5,5-difluoro-2-methylhexanoyl)-1-methylhydrazine-1-carboxylate

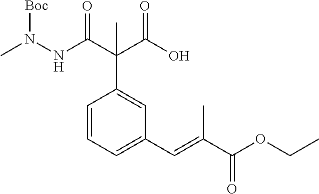 [SMILES: CCOC(=O)/C(C)=C/c1cccc(C(C)(C(=O)O)C(=O)NN(C)C(=O)OC(C)(C)C)c1]

To a stirred solution of (E)-6-(benzyloxy)-2-(3-(3-ethoxy-2-methyl-3-oxoprop-1-en-1-yl)phenyl)-5,5-difluoro-2-methylhexanoic acid (Intermediate 97D, 3 g, 6.51 mmol) in acetonitrile (60 mL) was added tert-butyl N-amino-N-methyl-carbamate (1.05 g, 7.17 mmol), 1-methylimidazole (1.87 g, 22.8 mmol) and N,N,N′,N′-tetramethylchloroformamidinium-hexafluorophosphate (1.83 g, 6.51 mmol) at room temperature. The reaction was stirred at room temperature for one hour and diluted with ethyl acetate (100 mL). The solution was washed with water, brine, dried over magnesium sulfate, and concentrated. The residue was purified by automated silica gel column chromatography (80 g silica gel column, eluting with 0-40% ethyl acetate in petroleum ether) to give the title compound (3.4 g, 89%) as an oil. MS (ESI): 611 m/z [M+Na]⁺, retention time: 2.05 minutes, purity: 95% (254 nm) (LC-MS Method 5).

Intermediate 97F: Tert-Butyl 2-(2-(3-(3-ethoxy-2-methyl-3-oxopropyl)phenyl)-5,5-difluoro-6-hydroxy-2-methylhexanoyl)-1-methylhydrazine-1-carboxylate

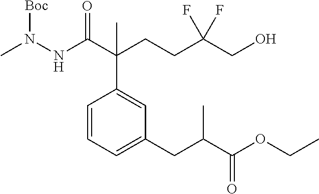 [SMILES: CCOC(=O)C(C)Cc1cccc(C(C)(CCC(F)(F)CO)C(=O)NN(C)C(=O)OC(C)(C)C)c1]

To a stirred solution of tert-butyl (E)-2-(6-(benzyloxy)-2-(3-(3-ethoxy-2-methyl-3-oxoprop-1-en-1-yl)phenyl)-5,5-difluoro-2-methylhexanoyl)-1-methylhydrazine-1-carboxylate (Intermediate 97E, 3.4 g, 5.78 mmol) in ethanol (30 mL) was added palladium on carbon (50% wet, 10%, 500 mg). The reaction mixture was stirred under hydrogen for 2 hours at 50° C., cooled to room temperature and filtered through a pad of Celite. The filter cake was washed with ethanol (50 mL). The filtrate was concentrated to give the crude title compound (2.6 g, 90%) as oil. MS (ESI): 523 m/z [M+Na]⁺, retention time: 1.80 minutes, purity: 80% (254 nm) (LC-MS Method 5).

Intermediate 97G: Tert-butyl 2-(6-((1-acetyl-1H-pyrazol-3-yl)oxy)-2-(3-(3-ethoxy-2-methyl-3-oxopropyl)phenyl)-5,5-difluoro-2-methylhexanoyl)-1-methylhydrazine-1-carboxylate

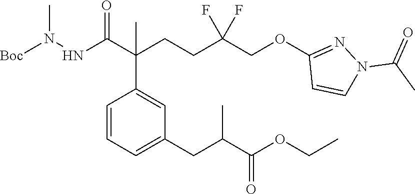 [SMILES: CCOC(=O)C(C)Cc1cccc(C(C)(CCC(F)(F)COc2ccn(C(C)=O)n2)C(=O)NN(C)C(=O)OC(C)(C)C)c1]

To a stirred solution of tert-butyl 2-(2-(3-(3-ethoxy-2-methyl-3-oxopropyl)phenyl)-5,5-difluoro-6-hydroxy-2-methylhexanoyl)-1-methylhydrazine-1-carboxylate (Intermediate 97F, 2.6 g, 5.19 mmol) in toluene (100 mL) was added 2-acetyl-1H-pyrazol-5-one (1.97 g, 15.6 mmol), triphenyl phosphine (4.09 g, 15.6 mmol) and diisopropyl azodicarboxylate (3.07 mL, 15.6 mmol). The reaction was stirred at 110° C. for 5 hours and concentrated. The residue was purified by automated flash chromatography (80 g silica gel column, eluting with 0-60% ethyl acetate in petroleum) to give the title compound (1.5 g, 47%) as oil. MS (ESI): 631 m/z [M+Na]⁺, retention time: 1.95 minutes, purity: 85% (254 nm) (LC-MS Method 5).

Intermediate 97H: Ethyl 3-(3-(6-((1-acetyl-1H-pyrazol-3-yl)oxy)-5,5-difluoro-2-methyl-1-(2-methylhydrazineyl)-1-oxohexan-2-yl)phenyl)-2-methylpropanoate

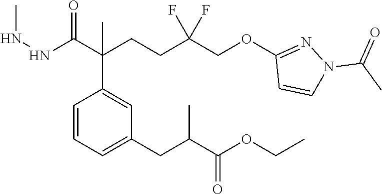 [SMILES: CCOC(=O)C(C)Cc1cccc(C(C)(CCC(F)(F)COc2ccn(C(C)=O)n2)C(=O)NNC)c1]

To a solution of tert-butyl 2-(6-((1-acetyl-1H-pyrazol-3-yl)oxy)-2-(3-(3-ethoxy-2-methyl-3-oxopropyl)phenyl)-5,5-difluoro-2-methylhexanoyl)-1-methylhydrazine-1-carboxylate (Intermediate 97G, 1.5 g, 2.46 mmol) in dichloromethane (10 mL) was added trifluoroacetic acid (1.83 mL, 24.6 mmol). The reaction was stirred at room temperature for four hours. The solvent was evaporated, and the residue was diluted with ethyl acetate (50 mL), washed with water, brine, dried over magnesium sulfate, and concentrated. The residue was purified by automated flash chromatography (20 g silica gel column, eluting with 0-50% ethyl acetate in petroleum) to give the title compound (0.8 g, 57%) as oil. MS (ESI): 531 m/z [M+Na]⁺, retention time: 1.66 minutes, purity: 85% (254 nm) (LC-MS Method 5).

The following intermediates were prepared based on the procedures described for Intermediate 97H.

| Inter. | | | MS m/z |
|---|---|---|---|
| No. | Structure | Name | [M + H]⁺ |
| 97H-1 | 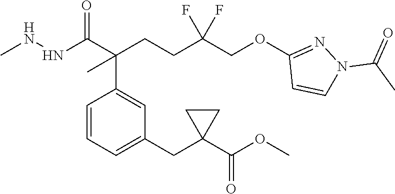 [SMILES: CNNC(=O)C(C)(CCC(F)(F)COc1ccn(C(C)=O)n1)c1cccc(CC2(C(=O)OC)CC2)c1] | methyl 1-(3-(6-((1-acetyl-1H-pyrazol-3-yl)oxy)-5,5- difluoro-2-methyl-1-(2-methylhydrazineyl)-1-oxohexan-2- yl)benzyl)cyclopropane-1-carboxylate | 507 |
| 97H-2 | 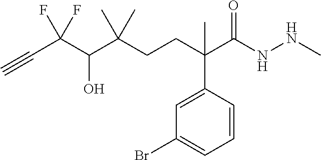 [SMILES: C#CC(F)(F)C(O)C(C)(C)CCC(C)(C(=O)NNC)c1cccc(Br)c1] | 2-(3-bromophenyl)-7,7-difluoro-6-hydroxy-N', 2,5,5- tetramethylnon-8-ynehydrazide | 431, 433 |

Intermediate 98: 1-Bromo-3-(1-bromo-3-((2,2-dimethylbut-3-yn-1-yl)oxy)propyl)benzene

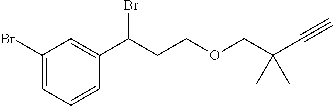 [SMILES: C#CC(C)(C)COCCC(Br)c1cccc(Br)c1]

To a stirred solution of 3-(3-bromo-3-(3-bromophenyl)propoxy)-2,2-dimethylpropanal (Intermediate 98E, 6.5 g, crude) in 100 mL of methanol was added potassium carbonate (7.3 g, 51 mmol) and dimethyl (1-diazo-2-oxopropyl)phosphonate (4.46 g, 23 mmol). The mixture was stirred at room temperature for 3 hours, then quenched with saturated ammonium chloride (100 mL), and extracted with ethyl acetate (2×50 mL). The combined organic phases were washed with saturated sodium bicarbonate, brine, dried over sodium sulfate and concentrated. The residue was purified by flash chromatography to afford the title compound (5 g, 75% for 2 steps). MS (ESI): 375 m/z [M+H]⁺, retention time: 2.51 minutes, purity: 95% (254 nm) (LC-MS Method 27).

The following intermediate was prepared based on the procedures described for Intermediate 98 and/or for Intermediates 98A to 98E.

| Inter. | | | MS m/z |
|---|---|---|---|
| No. | Structure | Name | [M + H]⁺ |
| 98-1 | 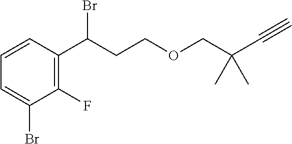 [SMILES: C#CC(C)(C)COCCC(Br)c1cccc(Br)c1F] | 1-bromo-3-(1-bromo-3-((2,2-dimethylbut-3-yn-1- yl)oxy)propyl)-2-fluorobenzene | 393 |

Intermediate 98A: 1-Bromo-3-(3-(chloromethoxy)propyl)benzene

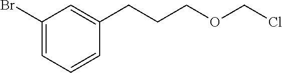 [SMILES: ClCOCCCc1cccc(Br)c1]

To a stirred solution of 3-(3-bromophenyl)propan-1-ol (25 g, 94.7 mmol) in dichloromethane (30 mL) was added paraformaldehyde (3.6 g, 120 mmol) and chlorotrimethylsilane (19.2 g, 180 mmol). The mixture was stirred at room temperature for 16 hours, then partitioned between water (100 mL) dichloromethane (50 mL). The separated organic phase, combined with two additional dichloromethane extracts (2×50 mL), was washed with brine (30 mL), dried over sodium sulfate, and concentrated. The crude title compound (23 g, crude) was used for the next step without further purification.

Intermediate 98B: Methyl 3-(3-(3-bromophenyl)propoxy)-2,2-dimethylpropanoate

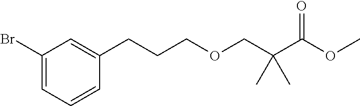 [SMILES: COC(=O)C(C)(C)COCCCc1cccc(Br)c1]

To a stirred and cooled (−78° C.) solution of methyl isobutyrate (10.2 g, 100 mmol) in tetrahydrofuran (200 mL) was added lithium diisopropylamide (2.5 M in tetrahydrofuran, 40 mL). The mixture was stirred for 15 minutes, treated with 1-bromo-3-(3-(chloromethoxy)propyl)-benzene (Intermediate 98A, 23 g, crude). The reaction was warmed to room temperature and stirred for 16 hours and quenched with saturated ammonium chloride (200 mL). The solution was extracted with ethyl acetate (2×100 mL). The combined organic phases were washed with brine (50 mL), dried over sodium sulfate, and concentrated. The residue was purified by chromatography to afford the title compound (20 g, 64% for 2 steps). MS (ESI): 329, 331 m/z [M+H]⁺, retention time: 2.45 minutes, purity: 90% (254 nm) (LC-MS Method 27).

Intermediate 98C: 3-(3-(3-Bromophenyl)propoxy)-2,2-dimethylpropan-1-ol

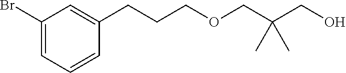 [SMILES: CC(C)(CO)COCCCc1cccc(Br)c1]

To a stirred and cooled (−40° C.) solution of methyl 3-(3-(3-bromophenyl)propoxy)-2,2-dimethylpropanoate (Intermediate 98B, 10 g, 30.5 mmol) in tetrahydrofuran (200 mL) was added lithium aluminum hydride (2.22 g, 61 mmol). The mixture was stirred at −20° C. for one hour, then quenched with saturated potassium sodium tartrate (100 mL) and stirred overnight. The solution was extracted with ethyl acetate (2×100 mL). The combined organic phases were washed with brine (50 mL), dried over sodium sulfate, and concentrated. The crude product was purified by chromatography to afford the title compound (9 g, 90%). MS (ESI): 301, 303 m/z [M+H]⁺, retention time: 2.24 minutes, purity: 90% (254 nm) (LC-MS Method 27).

Intermediate 98D: 3-(3-Bromo-3-(3-bromophenyl)propoxy)-2,2-dimethylpropan-1-ol

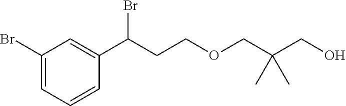 [SMILES: CC(C)(CO)COCCC(Br)c1cccc(Br)c1]

To a solution of 3-(3-(3-bromophenyl)propoxy)-2,2-dimethylpropan-1-ol (Intermediate 98C, 9 g, 29 mmol) in carbon tetrachloride (100 mL) was added 2,2′-azobis(2-methylpropionitrile) (450 mg, 2.9 mmol) and N-bromosuccinimide (6.16 g, 35 mmol). The mixture was stirred at 80° C. for one hour, cooled to room temperature and quenched with saturated sodium bicarbonate (200 mL). The solution was extracted with dichloromethane (2×50 mL). The combined organic phases were washed with brine (50 mL), dried over sodium sulfate, and concentrated. The residue was purified by chromatography to afford the title compound (6.5 g, 57%). MS (ESI): 381 m/z [M+H]⁺, retention time: 2.28 minutes, purity: 90% (254 nm) (LC-MS Method 27).

Intermediate 98E: 3-(3-Bromo-3-(3-bromophenyl)propoxy)-2,2-dimethylpropanal

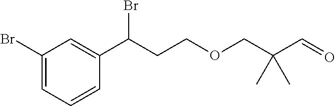 [SMILES: CC(C)(C=O)COCCC(Br)c1cccc(Br)c1]

To a stirred solution of 3-(3-bromo-3-(3-bromophenyl)propoxy)-2,2-dimethylpropan-1-ol (Intermediate 98D, 6.5 g, 17 mmol) in dichloromethane (50 mL) was added pyridinium chlorochromate (7.8 g, 34 mmol) and silica gel (8 g). The mixture was stirred at room temperature for 3 hours, diluted with petroleum ether (200 mL) and filtered. The filtrate was concentrated to afford the crude title compound (6.5 g, crude), which was used for the next step without further purification.

Intermediate 99: 2-(Chloromethyl)-5-(1,1-difluorobut-3-en-1-yl)pyridine

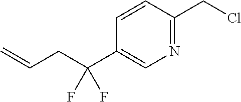

To a stirred solution of (5-(1,1-difluorobut-3-en-1-yl)pyridin-2-yl)methanol (2.5 g, 12.5 mmol) in dichloromethane (50 mL) was added sulfonyl chloride (3 g, 25.1 mmol). The mixture was stirred at room temperature for one hour and concentrated. The residue was diluted with ethyl acetate (100 mL), washed with saturated sodium bi-carbonate, brine, dried over sodium sulfate, and concentrated. The residue was purified by flash chromatography (40 g silica gel column, eluting with 0-15% ethyl acetate in petroleum ether) to give the title compound (2.5 g, 92%) as an oil. MS (ESI): 218 m/z [M+H]⁺, retention time: 1.79 minutes, purity: 95% (214 nm) (LC-MS Method 28).

Intermediate 99A: (5-(1,1-Difluorobut-3-en-1-ylpyridin-2-yl)methyl acetate

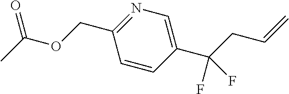 [SMILES: C=CCC(F)(F)c1ccc(COC(C)=O)nc1]

To a stirred solution of (5-(trifluoromethyl)pyridin-2-yl)methyl acetate (14 g, 63.87 mmol) in 1,2-dimethoxyethane (100 mL) was added 18-crown-6 (5.1 g, 19.16 mmol), cesium fluoride (0.97 g, 6.39 mmol), and allyltrimethylsilane (14.6 g, 127.7 mmol). The mixture was stirred at 80° C. overnight, cooled to room temperature, and quenched with water (300 mL). The solution was extracted with ethyl acetate (2×200 mL). The combined organic phases were washed with brine, dried over sodium sulfate, and concentrated. The residue was purified by automated flash chromatography (120 g silica gel column, eluting with 0-15% ethyl acetate in petroleum ether) to give the title compound (3 g, 19%) as an oil. MS (ESI): 242 m/z [M+H]⁺, retention time: 1.71 minutes, purity: 95% (254 nm) (LC-MS Method 28).

Intermediate 99B: (5-(1,1-Difluorobut-3-en-1-yl)pyridin-2-yl)methanol

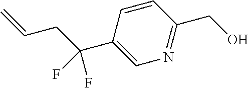

To a stirred solution of (5-(1,1-difluorobut-3-en-1-yl)pyridin-2-yl)methyl acetate (Intermediate 99A, 3.4 g, 14.1 mmol) in tetrahydrofuran (40 mL) and water (20 mL) was added lithium hydroxide monohydrate (1.18 g, 28.2 mmol). The mixture was stirred at room temperature for two hours, diluted with water (100 mL), and extracted with ethyl acetate (2×80 mL). The combined organic phases were washed with brine, dried over sodium sulfate, and concentrated to give the title compound (2.5 g, 89%) as an oil. MS (ESI): 200 m/z [M+H]⁺, retention time: 1.46 minutes, purity: 95% (214 nm) (LC-MS Method 28).

Intermediate 100: 6-((1-Acetyl-1H-pyrazol-3-yl)oxy)-5,5-difluoro-2-(3-((S)-3-methoxy-2-methyl-3-oxopropyl)phenyl)-2-methylhexanoic acid

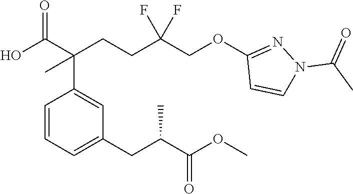 [SMILES: COC(=O)[C@@H](C)Cc1cccc(C(C)(CCC(F)(F)COc2ccn(C(C)=O)n2)C(=O)O)c1]

To a stirred solution of tert-butyl 6-((1-acetyl-1H-pyrazol-3-yl)oxy)-5,5-difluoro-2-(3-((S)-3-methoxy-2-methyl-3-oxopropyl)phenyl)-2-methylhexanoate (Intermediate 100C, 8.5 g, 16.2 mmol) in dichloromethane (60 mL) was added trifluoroacetic acid (18.5 g, 162 mmol) at room temperature. The reaction mixture was stirred at room temperature for two hours and concentrated. The residue was purified with automated flash chromatography (80 g silica gel column, eluting with 0-40% ethyl acetate in petroleum) to give the title compound (6.9 g, 91%). MS (ESI): 467 m/z [M+H]⁺, retention time: 1.36 minutes, purity: 95% (254 nm) (LC-MS Method 5).

The following intermediates were prepared based on the procedures described for Intermediate 100 and/or for Intermediates 100A to 100C.

| Inter. | | | MS m/z |
|---|---|---|---|
| No. | Structure | Name | [M + H]⁺ |
| 100-1 | 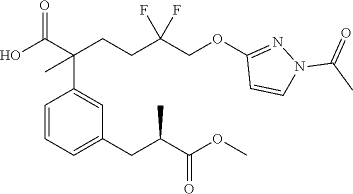 [SMILES: COC(=O)[C@H](C)Cc1cccc(C(C)(CCC(F)(F)COc2ccn(C(C)=O)n2)C(=O)O)c1] | 6-((1-acetyl-1H-pyrazol-3-yl)oxy)-5,5-difluoro-2-(3-((R)-3- methoxy-2-methyl-3-oxopropyl)phenyl)-2-methylhexanoic acid | 467 |
| 100-2 | 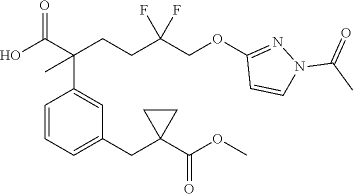 [SMILES: COC(=O)C1(Cc2cccc(C(C)(CCC(F)(F)COc3ccn(C(C)=O)n3)C(=O)O)c2)CC1] | 6-((1-acetyl-1H-pyrazol-3-yl)oxy)-5,5-difluoro-2-(3-((1- (methoxycarbonyl)cyclopropyl)methyl)phenyl)-2- methylhexanoic acid | 479 |
| 100-3 | 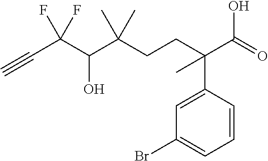 [SMILES: C#CC(F)(F)C(O)C(C)(C)CCC(C)(C(=O)O)c1cccc(Br)c1] | 2-(3-bromophenyl)-7,7-difluoro-6-hydroxy-2,5,5- trimethylnon-8-ynoic acid | 425, 427 [M + Na]⁺ |

Intermediate 100A: Tert-Butyl 6-(benzyloxy)-5,5-difluoro-2-(3-((S)-3-methoxy-2-methyl-3-oxopropyl)phenyl)-2-methylhexanoate

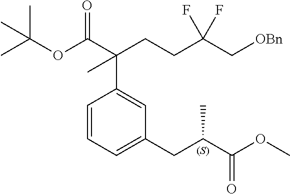

In a glove box, to a suspension of zinc (11 g, 169.8 mmol) in N,N-dimethylformamide (200 mL) was added iodine (718 mg, 2.83 mmol). The mixture was stirred at room temperature for 40 minutes, then treated with methyl (2R)-3-iodo-2-methyl-propanoate (9.7 g, 42.5 mmol), and stirred at room temperature for 40 minutes. To this mixture was added 2-dicyclohexylphosphino-2′,6′-dimethoxy-1,1′-biphenyl (1.16 g, 2.83 mmol) and tris(dibenzylideneacetone)dipalladium (1.3 g, 1.42 mmol), followed by tert-butyl 6-(benzyloxy)-5,5-difluoro-2-(3-iodophenyl)-2-methylhexanoate (Intermediate 97B-1, 15 g, 28.3 mmol). The reaction mixture was stirred at room temperature for 16 hours, then filtered. The filter cake was washed with ethyl acetate (200 mL). The combined filtrate was diluted with water (300 mL), extracted with ethyl acetate (2×200 mL). The combined extracts were washed with brine (2×300 mL), dried over sodium sulfate, and concentrated. The residue was purified by flash chromatography (330 g silica gel column, eluting with 0-10% ethyl acetate in petroleum ether) to give the title compound (12 g, 84%) as an oil. MS (ESI): 527 m/z [M+Na]⁺, retention time: 2.21 minutes, purity: 90% (214 nm) (LC-MS Method 5).

The following intermediate was prepared based on the procedures described for Intermediate 100A.

| Inter. | | | MS m/z |
|---|---|---|---|
| No. | Structure | Name | [M + H]⁺ |
| 100A-1 | 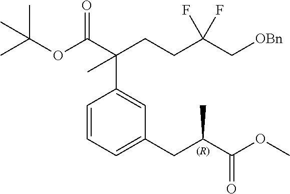 | tert-butyl 6-(benzyloxy)-5,5-difluoro-2-(3-((R)-3-methoxy-2- methyl-3-oxopropyl)phenyl)-2-methylhexanoate | 505 |

Intermediate 100B: Tert-Butyl 5,5-difluoro-6-hydroxy-2-(3-((S)-3-methoxy-2-methyl-3-oxopropyl)phenyl)-2-methylhexanoate

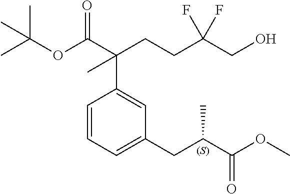

To a stirred solution of tert-butyl 6-(benzyloxy)-5,5-difluoro-2-(3-((S)-3-methoxy-2-methyl-3-oxopropyl)phenyl)-2-methylhexanoate (Intermediate 100A, 12 g, 23.8 mmol) in methanol (100 mL) was added palladium on carbon (50% wet, 10%, 2 g). The reaction mixture was stirred under hydrogen balloon for 2 hours at room temperature, then filtered through a pad of Celite. The filter cake was washed with methanol (100 mL). The combined filtrate was concentrated in vacuo to give the title compound (9 g, 91%) as an oil. MS (ESI): 437 m/z [M+Na]⁺, retention time: 1.89 minutes, purity: 85% (214 nm) (LC-MS Method 5).

The following intermediate was prepared based on the procedures described for Intermediate 100B.

| Inter. | | | MS m/z |
|---|---|---|---|
| No. | Structure | Name | [M + H]⁺ |
| 100B-1 | 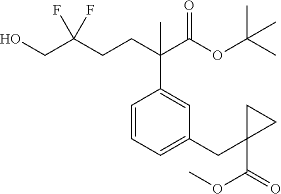 [SMILES: COC(=O)C1(Cc2cccc(C(C)(CCC(F)(F)CO)C(=O)OC(C)(C)C)c2)CC1] | methyl 1-(3-(1-(tert-butoxy)-5,5-difluoro-6-hydroxy-2-methyl- 1-oxohexan-2-yl)benzyl)cyclopropane-1-carboxylate | 449 [M + Na]⁺ |
| 100B-2 | 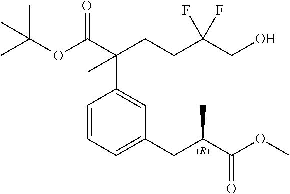 | tert-butyl 5,5-difluoro-6-hydroxy-2-(3-((R)-3-methoxy-2- methyl-3-oxopropyl)phenyl)-2-methylhexanoate | 415 [M + Na]⁺ |

Intermediate 100C: Tert-Butyl 6-((1-acetyl-1H-pyrazol-3-yl)oxy)-5,5-difluoro-2-(3-((S)-3-methoxy-2-methyl-3-oxopropyl)phenyl)-2-methylhexanoate

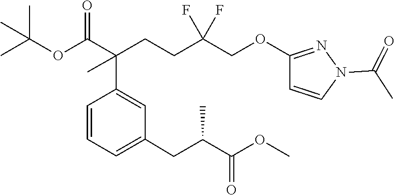 [SMILES: COC(=O)[C@@H](C)Cc1cccc(C(C)(CCC(F)(F)COc2ccn(C(C)=O)n2)C(=O)OC(C)(C)C)c1]

To a stirred solution of tert-butyl 5,5-difluoro-6-hydroxy-2-(3-((S)-3-methoxy-2-methyl-3-oxopropyl)phenyl)-2-methylhexanoate (Intermediate 100B, 9 g, 21.7 mmol) in toluene (150 mL) was added 1-acetyl-1,2-dihydro-3H-pyrazol-3-one (8.2 g, 65.1 mmol), triphenylphosphine (17 g, 65.1 mmol) and diisopropyl azodicarboxylate (13 g, 65.1 mmol). The reaction mixture was stirred at 110° C. for 5 hours and concentrated. The residue was purified by automated flash chromatography (330 g silica gel column, eluting with 0-30% ethyl acetate in petroleum) to give the title compound (8.5 g, 75%) as an oil. MS (ESI): 545 m/z [M+Na]⁺, retention time: 1.56 minutes, purity: 93% (254 nm) (LC-MS Method 5).

The following intermediate was prepared based on the procedures described for Intermediate 100C.

| Inter. | | | MS m/z |
|---|---|---|---|
| No. | Structure | Name | [M + H]⁺ |
| 100C-1 | 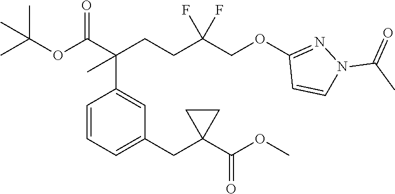 [SMILES: COC(=O)C1(Cc2cccc(C(C)(CCC(F)(F)COc3ccn(C(C)=O)n3)C(=O)OC(C)(C)C)c2)CC1] | methyl 1-(3-(6-((1-acetyl-1H- pyrazol-3-yl)oxy)-1-(tert- butoxy)-5,5-difluoro-2-methyl-1-oxohexan-2- yl)benzyl)cyclopropane-1-carboxylate | 557 [M + Na]⁺ |
| 100C-2 | 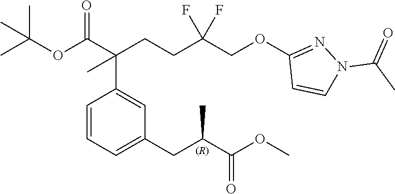 | tert-butyl 6-((1-acetyl-1H-pyrazol- 3-yl)oxy)-5,5-difluoro- 2-(3-((R)-3-methoxy-2-methyl- 3-oxopropyl)phenyl)-2- methylhexanoate | 523 |

Intermediate 101: Methyl (2S)-3-(3-(6-((1H-pyrazol-3-yl)oxy)-5,5-difluoro-2-methyl-1-(2-methylhydrazineyl)-1-oxohexan-2-yl)phenyl)-2-methylpropanoate

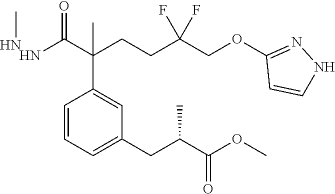

To a stirred solution of benzyl 2-(6-((1H-pyrazol-3-yl)oxy)-5,5-difluoro-2-(3-((S)-3-methoxy-2-methyl-3-oxopropyl)phenyl)-2-methylhexanoyl)-1-methylhydrazine-1-carboxylate (Intermediate 101B, 5.3 g, 9 mmol) in methanol (50 mL) was added palladium on carbon (50% wet, 10%, 1 g). The reaction mixture was stirred under hydrogen for 2 hours at 50° C., cooled to room temperature and filtered through a pad of Celite. The filtrate was concentrated to give the title compound (3.9 g, 95%) as oil. MS (ESI): 453 m/z [M+H]⁺, retention time: 1.61 minutes, purity: 92% (254 nm) (LC-MS Method 5).

The following intermediates were prepared based on the procedures described for Intermediate 101.

| Inter. | | | MS m/z |
|---|---|---|---|
| No. | Structure | Name | [M + H]⁺ |
| 101-1 | 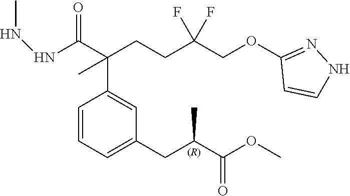 | methyl (2R)-3-(3-(6-((1H-pyrazol-3-yl)oxy)-5,5-difluoro-2- methyl-1-(2-methylhydrazineyl)-1-oxohexan-2-yl)phenyl)-2- methylpropanoate | 453 |

Intermediate 101A: Benzyl 2-(6-((1-acetyl-1H-pyrazol-3-yl)oxy)-5,5-difluoro-2-(3-((S)-3-methoxy-2-methyl-3-oxopropyl)phenyl)-2-methylhexanoyl)-1-methylhydrazine-1-carboxylate

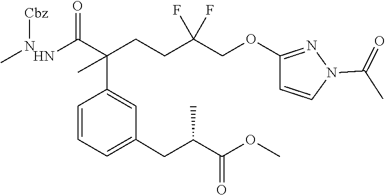

To a stirred solution of 6-((1-acetyl-1H-pyrazol-3-yl)oxy)-5,5-difluoro-2-(3-((S)-3-methoxy-2-methyl-3-oxopropyl)phenyl)-2-methylhexanoic acid (Intermediate 100, 4.6 g, 9.9 mmol) in acetonitrile (50 mL) was added benzyl 1-methylhydrazine-1-carboxylate (1.9 g, 10.8 mmol), 1-methylimidazole (2.8 g, 34.6 mmol) and N,N,N′,N′-tetramethylchloroformamidinium-hexafluorophosphate (2.8 g, 9.9 mmol) at room temperature. The reaction mixture was stirred at room temperature for one hour, then diluted with ethyl acetate (150 mL) and washed with water, brine, dried over magnesium sulfate, and concentrated. The residue was purified by silica gel column chromatography (80 g silica gel column, eluting with 0-50% ethyl acetate in petroleum ether) to give the title compound (6 g, 96%) as an oil. MS (ESI): 629 m/z [M+H]⁺, retention time: 1.93 minutes, purity: 95% (254 nm) (LC-MS Method 5).

The following intermediate was prepared based on the procedures described for Intermediate 101A.

| Inter. | | | |
|---|---|---|---|
| No. | Structure | Name | MS m/z [M + H]⁺ |
| 101A-1 | 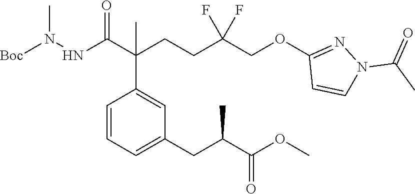 [SMILES: COC(=O)[C@H](C)Cc1cccc(C(C)(CCC(F)(F)COc2ccn(C(C)=O)n2)C(=O)NN(C)C(=O)OC(C)(C)C)c1] | tert-butyl 2-(6-((1-acetyl-1H-pyrazol-3- yl)oxy)-5,5-difluoro-2-(3-((R)-3- methoxy-2-methyl-3-oxopropyl)phenyl)- 2-methylhexanoyl)-1-methylhydrazine- 1-carboxylate | 617 [M + Na]⁺ |
| 101A-2 | 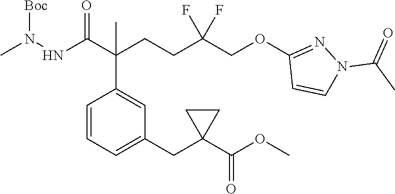 [SMILES: COC(=O)C1(Cc2cccc(C(C)(CCC(F)(F)COc3ccn(C(C)=O)n3)C(=O)NN(C)C(=O)OC(C)(C)C)c2)CC1] | tert-butyl 2-(6-((1-acetyl-1H-pyrazol-3- yl)oxy)-5,5-difluoro-2-(3-((1- (methoxycarbonyl)cyclopropyl)methyl)p henyl)-2-methylhexanoyl)-1- methylhydrazine-1-carboxylate | 629 [M + Na]⁺ |

Intermediate 101B: Benzyl 2-(6-((1H-pyrazol-3-yl)oxy)-5,5-difluoro-2-(3-((S)-3-methoxy-2-methyl-3-oxopropyl)phenyl)-2-methylhexanoyl)-1-methylhydrazine-1-carboxylate

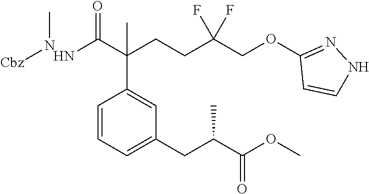

To a stirred solution of benzyl 2-(6-((1-acetyl-1H-pyrazol-3-yl)oxy)-5,5-difluoro-2-(3-((S)-3-methoxy-2-methyl-3-oxopropyl)phenyl)-2-methylhexanoyl)-1-methylhydrazine-1-carboxylate (Intermediate 101A, 6 g, 9.5 mmol) in methanol (50 mL) was added potassium carbonate (2.6 g, 19 mmol) at room temperature. The reaction mixture was stirred at room temperature for two hours, then diluted with ethyl acetate (150 mL). The solution was washed with water, brine, dried over magnesium sulfate, and concentrated to give the title compound (5.3 g, 95%) as oil. MS (ESI): 587 m/z [M+H]⁺, retention time: 1.81 minutes, purity: 92% (254 nm) (LC-MS Method 5).

The following intermediate was prepared based on the procedures described for Intermediate 101B.

| | | | MS m/z |
|---|---|---|---|
| Inter. No. | Structure | Name | [M + H]⁺ |
| 101B-1 | 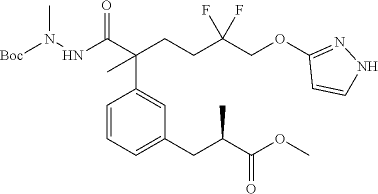 [SMILES: COC(=O)[C@H](C)Cc1cccc(C(C)(CCC(F)(F)COc2cc[nH]n2)C(=O)NN(C)C(=O)OC(C)(C)C)c1] | tert-butyl 2-(6-((1H-pyrazol-3-yl) oxy)-5,5-difluoro-2-(3-((R)- 3-methoxy-2-methyl-3-oxopropyl)phenyl)-2- methylhexanoyl)-1-methylhydrazine-1-carboxylate | 553 [M + Na]⁺ |

Intermediate 102: 2-Fluoro-5-((6-fluoro-4-(hydroxymethyl)-1-(phenylsulfonyl)-1H-indol-5-yl)oxy)benzonitrile

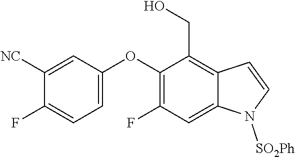

To a stirred solution of (5-(3-cyano-4-fluorophenoxy)-6-fluoro-1-(phenylsulfonyl)-1H-indol-4-yl)methyl acetate (Intermediate 87, 3 g, 6.2 mmol) in methanol (30 mL) was added potassium carbonate (1.7 g, 12.4 mmol). The reaction was stirred at room temperature for two hours, then diluted with ethyl acetate (100 mL). The mixture was washed with brine, dried over sodium sulfate, and concentrated. The residue was purified by automated flash chromatography 40 g silica gel column, eluting with 0-50% ethyl acetate in petroleum ether) to give the title compound (2 g, 73%) as oil. MS (ESI): 423 m/z [M−H₂O+H]⁺, retention time: 1.88 minutes, purity: 85% (254 nm) (LC-MS Method 5).

Intermediate 103: 2-Fluoro-5-((6-fluoro-1-(triisopropylsilyl)-4-vinyl-1H-indol-5-yl)((tetrahydro-2H-pyran-2-yl)oxy)methyl)benzonitrile

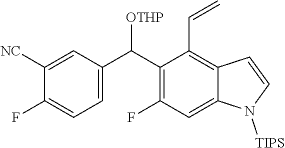

To a stirred and degassed solution of 5-((4-bromo-6-fluoro-1-(triisopropylsilyl)-1H-indol-5-yl)((tetrahydro-2H-pyran-2-yl)oxy)methyl)-2-fluorobenzonitrile (Intermediate 103C, 18 g, 29.8 mmol) and 4,4,5,5-tetramethyl-2-vinyl-1,3,2-dioxaborolane (6.06 mL, 35.8 mmol) in dioxane/water(5:1) (150 mL) was added tris(dibenzylideneacetone)dipalladium(0) (1.71 g, 0.16 mmol), potassium carbonate (13.3 g, 89.5 mmol) and tri-tert-butylphosphine tetrafluoroborate (1.73 g, 5.96 mmol). The suspension was stirred at 50° C. for 1 hour. The reaction was diluted with water and extracted with ethyl acetate (100 mL). The combined organic layers were washed with brine, dried over sodium sulphate, and concentrated. The residue was purified by automated flash chromatography (120 g silica gel column, eluting with 0-20% ethyl acetate in Petroleum ether) to give the title compound (13.28 g, 84%) as a solid. MS (ESI): 573 m/z [M+Na]⁺, retention time: 2.85 and 2.97 minutes (two diastereomers), purity: 47%+48% (two diastereomers) (254 nm) (LC-MS Method 26).

Intermediate 103A: 4-Bromo-6-fluoro-1-(triisopropylsilyl)-1H-indole

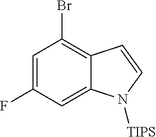

To a stirred and cooled (0° C.) solution of 4-bromo-6-fluoro-1H-indole (29 g, 0.1 mol) in tetrahydrofuran (300 mL) was slowly added sodium hydride (8.67 g, 130 mol). The mixture was stirred for 2 hours, then treated with chloro(triisopropyl)silane (25.5 mL, 0.119 mol) at 0° C. The reaction was stirred for another 2 hours at 0° C., quenched with 500 mL of water and extracted with ethyl acetate (3×200 mL). The combined organic phase was washed with brine, dried over sodium sulfate, and concentrated. The residue was purified by automated flash chromatography (330 g silica gel column, 0-10% ethyl acetate in petroleum ether, UV 254 nm) to give the title compound (34 g, 80%) as a colorless oil. MS (ESI): 370, 372 m/z [M+H]⁺.

Intermediate 103B:5-((4-Bromo-6-fluoro-1-(triisopropylsilyl)-1H-indol-5-yl)(hydroxy)methyl)-2-fluorobenzonitrile

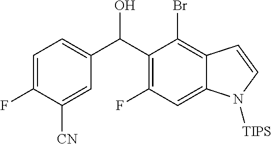

To a stirred and cooled (−78° C.) solution of 4-bromo-6-fluoro-1-(triisopropylsilyl)-1H-indole (Intermediate 103A, 11.1 g, 30 mmol) in 150 mL of tetrahydrofuran was added lithium diisopropylamide (18 mL, 36 mmol, 2N in tetrahydrofuran) dropwise. The reaction was stirred at −78° C. for 1 hour, then treated with 2-fluoro-5-formyl-benzonitrile (4.47 g, 30 mmol). The mixture was slowly warmed to room temperature and stirred for 10 hours, quenched with 300 mL of water, and extracted with ethyl acetate (3×150 mL). The combined organic phase was washed with brine, dried over sodium sulfate, and concentrated. The residue was purified by silica gel column chromatography (petroleum ether:ethyl acetate=5:1) to give the title compound (9.8 g, 63%) as a yellow oil. MS (ESI): 519, 521 m/z [M+H]⁺.

Intermediate 103C: 5-((4-Bromo-6-fluoro-1-(triisopropylsilyl)-1H-indol-5-yl)((tetrahydro-2H-pyran-2-yl)oxy)methyl)-2-fluorobenzonitrile

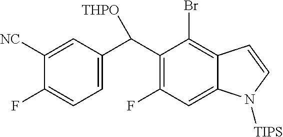

To a stirred solution of 5-((4-bromo-6-fluoro-1-(triisopropylsilyl)-1H-indol-5-yl)(hydroxy)methyl)-2-fluorobenzonitrile (Intermediate 103B, 18 g, 34.6 mmol) in dichloromethane(150 mL) was added 3,4-dihydro-2H-pyran (12.6 mL, 0.128 mol) and pyridinium 4-methylbenzenesulfonate (1.74 g, 6.92 mmol). The reaction was stirred at room temperature overnight and diluted with 100 mL of dichloromethane. The solution was washed with brine, dried over magnesium sulfate, and concentrated. The residue was purified by automated flash chromatography (120 g silica gel column, eluting with 0-30% ethyl acetate in petroleum) to give the title compound (18 g, 86%) as solid. MS (ESI): 625, 627 m/z [M+Na]⁺, retention time: 2.94 and 3.01 minutes (two diastereomers), purity: 51%+46% (two diastereomers) (254 nm) (LC-MS Method 26).

Intermediate 104: Methyl 1-(3-(2-(tert-butoxy)-2-oxoethyl)benzyl)cyclopropane-1-carboxylate

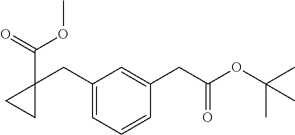 [SMILES: COC(=O)C1(Cc2cccc(CC(=O)OC(C)(C)C)c2)CC1]

In a glove box, to a suspension of zinc (17 g, 260.4 mmol) in tetrahydrofuran (200 mL) was added iodine (1.1 g, 4.34 mmol). The mixture was stirred at room temperature for 5 minutes, treated with tert-butyl 2-bromoacetate (25.4 g, 130.2 mmol). The mixture was stirred at room temperature for two hours, then transferred to a degassed solution of methyl 1-(3-iodobenzyl)cyclopropane-1-carboxylate (Intermediate 104B, 14 g, 43.4 mmol), bis(tri-t-butylphosphine)palladium(0) (1.1 g, 2.17 mmol) under Ar. The reaction was stirred at 75° C. for 16 hours, cooled to room temperature and filtered through a pad of Celite. The filter cake was washed with ethyl acetate (300 mL). The combined filtrate was concentrated. The residue was purified by automated flash chromatography (120 g silica gel column, eluting with 0-10% ethyl acetate in petroleum ether for 30 min) to give the title compound (11.6 g, 86%) as an oil. MS (ESI): 327 m/z [M+Na]⁺, retention time: 2.00 minutes, purity: 90% (254 nm) (LC-MS Method 5).

Intermediate 104A: Tert-Butyl 1-(3-iodobenzyl)cyclopropane-1-carboxylate

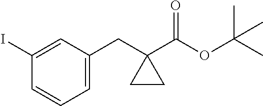 [SMILES: CC(C)(C)OC(=O)C1(Cc2cccc(I)c2)CC1]

To a stirred and cooled (−78° C.) solution of tert-butyl cyclopropanecarboxylate (20 g, 140.6 mmol) in tetrahydrofuran (200 mL) was added lithium diisopropylamide (2.0 M, 85 mL, 170 mmol) dropwise over 15 minutes. The mixture was stirred for 1 h at −78° cand treated dropwise with a solution of 1-(bromomethyl)-3-iodobenzene (45.9 g, 154.7 mmol) in tetrahydrofuran (50 mL). The reaction was warmed up to room temperature and stirred for 16 hours, then quenched with water (500 mL). The solution was extracted with ethyl acetate (2×200 mL). The combined organic extracts were washed with brine, dried over sodium sulfate, and concentrated. The residue was purified by flash chromatography (330 g silica gel column, eluting with 0-10% ethyl acetate in petroleum ether) to give the title compound (38 g, 75%) as an oil. MS (ESI): 381 m/z [M+Na]⁺, retention time: 2.21 minutes, purity: 90% (254 nm) (LC-MS Method 5).

Intermediate 104B: Methyl 1-(3-iodobenzyl)cyclopropane-1-carboxylate

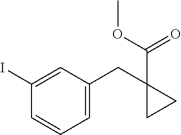

To a solution of tert-butyl 1-(3-iodobenzyl)cyclopropane-1-carboxylate (Intermediate 104A, 38 g, 106.0 mmol) in hydrogen chloride/methanol (265 mL) was stirred at 50° C. for two hours and concentrated. The residue was purified with automated flash chromatography (330 g silica gel column, eluting with 0-10% ethyl acetate in petroleum) to give methyl 1-(3-iodobenzyl)-cyclopropane-1-carboxylate (27 g, 80%). MS (ESI): 317 m/z [M+H]⁺, retention time: 2.01 minutes, purity: 90% (254 nm) (LC-MS Method 5).

Intermediate 105: 5,5′-Disulfanediylbis(2-fluorobenzonitrile)

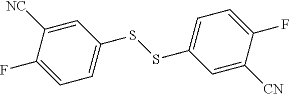 [SMILES: N#Cc1cc(SSc2ccc(F)c(C#N)c2)ccc1F]

To a stirred solution of 2-fluoro-5-((4-methoxybenzyl)thio)benzonitrile (Intermediate 105A, 25 g, 91.5 mmol) in anisole (100 mL) was added trifluoroacetic acid (100 mL). The reaction was stirred at 50° C. overnight and concentrated. To the oily residue was added dimethyl sulfoxide (13 mL) and iodine (1.16 g, 4.57 mmol). After stirring at room temperature overnight, the mixture was diluted with ethyl acetate (150 mL). The solution was washed with brine, dried over sodium sulfate, and concentrated. The residue was purified by automated flash chromatography (330 g silica gel column, eluting with 0-20% ethyl acetate in petroleum ether) to give the title compound (8.8 g, 63%) as a solid. MS (ESI): 327 m/z [M+Na]⁺, retention time: 1.93 minutes, purity: 82% (254 nm) (LC-MS Method 2).

Intermediate 105A: 2-fluoro-5-((4-methoxybenzyl)thio)benzonitrile

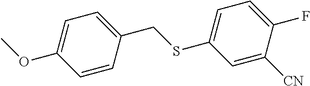 [SMILES: COc1ccc(CSc2ccc(F)c(C#N)c2)cc1]

To a stirred and degassed solution of 5-bromo-2-fluoro-benzonitrile (27 g, 135 mmol) and (4-methoxyphenyl)methanethiol (25 g, 162 mmol) in dioxane (300 mL) was added tris(dibenzylideneacetone)dipalladium(0) (6.18 g, 6.75 mmol), Xantphos (7.81 g, 13.5 mmol) and N,N-diisopropylethylamine (46.2 mL, 270 mmol). The reaction was stirred at 100° C. overnight and concentrated. The residue was purified by flash chromatography (2×330 g silica gel column, eluting with 0-10% ethyl acetate in petroleum ether) to give the title compound (25 g, 68%) as solid. MS (ESI): 274 m/z [M+H]⁺, retention time: 1.86 minutes, purity: 99% (254 nm) (LC-MS Method 2).

Intermediate 106: 2-Fluoro-5-((6-fluoro-4-methyl-1-(phenylsulfonyl)-1H-indol-5-yl)thio)benzonitrile

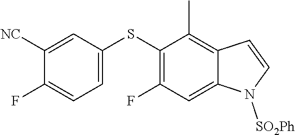

To a stirred and cooled (0° C.) solution of 2-fluoro-5-((6-fluoro-4-methyl-1H-indol-5-yl)thio)benzonitrile (Intermediate 106C, 2.3 g, 7.6 mmol) in N,N-dimethylformamide (20 mL) was added sodium hydride (60%, 0.37 g, 9.2 mmol) and benzenesulfonyl chloride (1.48 g, 8.4 mmol). The reaction was stirred at 0° C. for two hours, then quenched with water (50 mL). The mixture was extracted with ethyl acetate (3×50 mL). The combined organic extracts were washed with brine, dried over sodium sulfate, and concentrated. The residue was purified by flash chromatography (330 g silica gel column, eluting with 0-20% ethyl acetate in petroleum ether) to give the title compound (2.8 g, 83%) as a solid. MS (ESI): 441 m/z [M+H]⁺, retention time: 1.59 minutes, purity: 81% (214 nm) (LC-MS Method 24).

Intermediate 106A: 5-((4-Bromo-6-fluoro-1-(triisopropylsilyl)-1H-indol-5-yl)thio)-2-fluorobenzonitrile

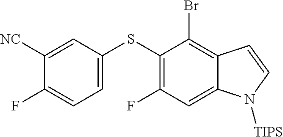

To a stirred and cooled (−78° C.) solution of 4-bromo-6-fluoro-1-(triisopropylsilyl)-1H-indole (103A, 4.8 g, 13 mmol) in tetrahydrofuran (65 mL) was added lithium diisopropylamide (2 M in tetrahydrofuran, 13 mL, 26 mmol). The mixture was stirred at −78° C. for 2 hours, then treated with 5,5′-disulfanediylbis(2-fluorobenzonitrile) (Intermediate 105, 5.92 g, 19.4 mmol). The reaction was stirred at −78° for 2 hours, warmed to room temperature for one hour, and quenched with saturated ammonium chloride (150 mL). The mixture was extracted with ethyl acetate (3×80 mL). The combined organic extracts were washed with brine, dried over sodium sulfate, and concentrated. The residue was purified by automated flash chromatography (120 g silica gel column, eluting with 0-20% ethyl acetate in petroleum ether) to give the title compound (4.3 g, 64%) as a solid. MS (ESI): 521, 523 m/z [M+H]⁺, retention time: 1.80 minutes, purity: 93% (214 nm) (LC-MS Method 24).

Intermediate 106B: 2-Fluoro-5-((6-fluoro-4-methyl-1-(triisopropylsilyl)-1H-indol-5-yl)thio)benzonitrile

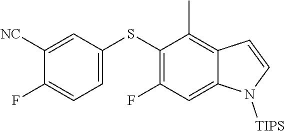

To a stirred and degassed solution of 5-((4-bromo-6-fluoro-1-(triisopropylsilyl)-1H-indol-5-yl)thio)-2-fluorobenzonitrile (Intermediate 106A, 5.5 g, 10.5 mmol) in dioxane/water (10:1) (50 mL) was added [1,1′-bis(diphenylphosphino)ferrocene]dichloropalladium (II) (773 mg, 1.05 mmol), sodium bicarbonate (2.65 g, 31.5 mmol) and methylboronic acid (3.15 g, 52.5 mmol). The mixture was stirred at 90° C. for 16 hours, cooled to room temperature, and diluted with ethyl acetate (150 mL). The solution was washed with brine, dried over sodium sulfate, and concentrated. The residue was purified by flash chromatography (330 g silica gel column, eluting with 0-20% ethyl acetate in petroleum ether) to give the title compound (3.4 g, 70%) as solid. MS (ESI): 457 m/z [M+H]⁺, retention time: 1.81 minutes, purity: 93% (214 nm) (LC-MS Method 2).

Intermediate 106C: 2-Fluoro-5-((6-fluoro-4-methyl-1H-indol-5-yl)thio)benzonitrile

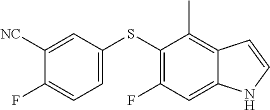 [SMILES: Cc1c(Sc2ccc(F)c(C#N)c2)c(F)cc2[nH]ccc12]

To a solution of 2-fluoro-5-(6-fluoro-4-methyl-1-(triisopropylsilyl)-1H-indol-5-yl)thio)-benzonitrile (Intermediate 106B, 4 g, 8.77 mmol) in tetrahydrofuran (30 mL) was added tetra-n-butylammonium fluoride (48.3 mL, 43.8 mmol). The mixture was stirred at 40° C. for four hours, cooled to room temperature, and diluted with ethyl acetate (150 mL). The solution was washed with brine (3×50 mL), dried over sodium sulfate, and concentrated. The residue was purified by flash chromatography (330 g silica gel column, eluting with 0-20% ethyl acetate in petroleum ether) to give the title compound (2.3 g, 90%) as solid. MS (ESI): 301 m/z [M+H]⁺, retention time: 1.65 minutes, purity: 86% (214 nm) (LC-MS Method 2).

Intermediate 107: 2-(3-bromophenyl)-5-(2-(ethoxycarbonyl)cyclopropyl)-2-methylpentanoic acid

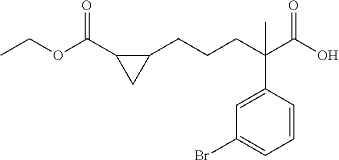 [SMILES: CCOC(=O)C1CC1CCCC(C)(C(=O)O)c1cccc(Br)c1]

A mixture of ethyl 2-(4-(3-bromophenyl)-4-methyl-5-oxo-5-(2-(trimethylsilyl)ethoxy)pentyl)-cyclopropane-1-carboxylate (Intermediate 107B, 3.8 g, 4.72 mmol) in tetra-n-butyl ammonium fluoride (2 M in tetrahydrofuran, 15 mL, 30 mmol) was stirred at room temperature for 2 hours, then quenched with water (200 ml), extracted with ethyl acetate (3×100 mL). The combined organic extracts were washed with brine, dried over sodium sulfate, and concentrated. The residue was purified by silica gel column chromatography (eluting with 20% methanol in dichloromethane) to afford the title compound (1.6 g, 49% two steps). 1H NMR (400 MHz, CDCl₃) δ 7.50 (t, J=1.7 Hz, 1H), 7.40 (d, J=7.8 Hz, 1H), 7.30 (d, J=8.2 Hz, 1H), 7.21 (t, J=7.9 Hz, 1H), 4.10 (q, J=7.1 Hz, 2H), 2.11-1.81 (m, 2H), 1.63-1.49 (m, 3H), 1.37-1.19 (m, 9H), 1.14 (d, J=3.4 Hz, 1H), 0.67-0.64 (m, 1H).

Intermediate 107A: 1-Ethyl 8-(2-(trimethylsilyl)ethyl) (Z)-7-(3-bromophenyl)-7-methyloct-2-enedioate

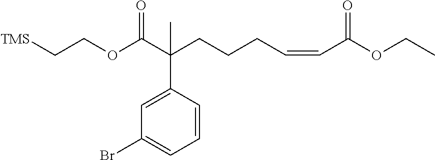 [SMILES: CCOC(=O)/C=C\CCCC(C)(C(=O)OCC[Si](C)(C)C)c1cccc(Br)c1]

To a stirred solution of 2-(trimethylsilyl)ethyl 2-(3-bromophenyl)-2-methylhept-6-enoate (Intermediate 7A-36, 10 g, 25.2 mmol) in 150 ml of 1,2-dichloroethane was added ethyl acrylate (15.1 g, 151 mmol), Grubbs catalyst 2nd generation (3.2 g, 3.77 mmol). The reaction was refluxed for 24 hours and concentrated. The residue was purified by silica gel column chromatography (eluting with 40% dichloromethane in petroleum ether) to afford the title compound (10 g, 76%). ¹H NMR (400 MHz, CDCl₃) δ 7.45 (d, J=1.8 Hz, 1H), 7.39 (dt, J=7.0, 1.8 Hz, 1H), 7.27-7.15 (m, 2H), 6.99-6.82 (m, 1H), 5.81 (d, J=15.6 Hz, 1H), 4.26-4.06 (m, 4H), 2.29-2.14 (m, 2H), 2.08-1.78 (m, 2H), 1.53 (d, J=8.8 Hz, 3H), 1.44-1.32 (m, 3H), 0.98-0.79 (m, 4H), 0.00 (s, 9H).

Intermediate 107B: Ethyl 2-(4-(3-bromophenyl)-4-methyl-5-oxo-5-(2-(trimethylsilyl)ethoxy)pentyl)cyclopropane-1-carboxylate

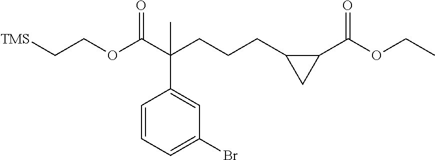 [SMILES: CCOC(=O)C1CC1CCCC(C)(C(=O)OCC[Si](C)(C)C)c1cccc(Br)c1]

To a suspension of potassium tert-butoxide (1.58 g, 14.1 mmol) in dimethyl sulfoxide (40 mL) was added trimethylsulfoxonium iodide (3.38 g, 15.3 mmol). The mixture was stirred for 2 hours, then treated with a solution of 1-ethyl 8-(2-(trimethylsilyl)ethyl) (Z)-7-(3-bromophenyl)-7-methyloct-2-enedioate (Intermediate 107A, 4 g, 8.52 mmol) in dimethyl sulfoxide (5 mL) dropwise. The reaction mixture was stirred overnight, quenched with water (200 mL), and extracted with ethyl acetate (3×200 mL). The combined organic extracts were washed with brine, dried over sodium sulfate, and concentrated. The residue was used for the next step without further purification. (3.8 g, crude). MS (ESI): 505, 507 m/z [M+Na]⁺, retention time: 2.66 minutes, purity: 50% (214 nm) (LC-MS Method 18).

Intermediate 108: Tert-Butyl 2-(3-bromophenyl)-7,7-difluoro-6-hydroxy-2,5,5-trimethylnon-8-ynoate

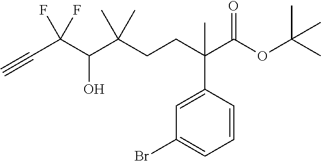 [SMILES: C#CC(F)(F)C(O)C(C)(C)CCC(C)(C(=O)OC(C)(C)C)c1cccc(Br)c1]

To a stirred solution of tert-butyl 2-(3-bromophenyl)-7,7-difluoro-6-hydroxy-2,5,5-trimethyl-9-(triisopropylsilyl)non-8-ynoate (Intermediate 108A, 6.5 g, 10.6 mmol) in dichloromethane (100 mL) was added a solution of tetrabutylammonium fluoride (1 M in tetrahydrofuran, 15.8 mL, 15.8 mmol). The mixture was stirred at room temperature for 1 hour and concentrated. The residue was diluted with water (100 mL), extracted with ethyl acetate (3×100 mL). The combined organic phases were washed with brine (20 mL), dried over sodium sulfate, and concentrated. The residue was purified by automated flash chromatography (80 g silica gel column, eluting with 0-30% ethyl acetate in petroleum ether) to afford the title compound (2.4 g, 49%). MS (ESI): 459, 461 m/z [M+Na]⁺, retention time: 1.73+1.77 minutes (two diastereomers), purity: 35%+47% (214 nm) (LC-MS Method 32).

Intermediate 108A: Tert-butyl 2-(3-Bromophenyl)-7,7-difluoro-6-hydroxy-2,5,5-trimethyl-9-(triisopropylsilyl)non-8-ynoate

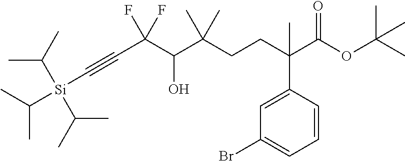 [SMILES: CC(C)[Si](C#CC(F)(F)C(O)C(C)(C)CCC(C)(C(=O)OC(C)(C)C)c1cccc(Br)c1)(C(C)C)C(C)C]

To a stirred solution of tert-butyl 2-(3-bromophenyl)-2,5,5-trimethyl-6-oxohexanoate (8 g, 20.9 mmol) and (3-bromo-3,3-difluoro-prop-1-ynyl)-triisopropyl-silane (13 g, 41.8 mmol) in tetrahydrofuran (200 mL) was added zinc dust (5.84 g, 104 mmol). The mixture was treated with 0.5 mL of trimethyl chlorosilane dropwise and stirred at room temperature for 16 hours, then filtered. The filtrate was diluted with water (200 mL) and extracted with ethyl acetate (3×100 mL). The combined organic phases were washed with brine, dried over sodium sulfate, and concentrated. The residue was purified by automated flash chromatography (120 g silica gel column, eluting with 0-30% ethyl acetate in petroleum ether) to afford the title compound as an oil (6.5 g, 51%). MS (ESI): 637, 639 m/z [M+Na]⁺, retention time: 2.49 minutes, purity: 57% (214 nm) (LC-MS Method 31).

Intermediate 109: 5-((4-((5-Allylpyrazin-2-yl)methyl)-6-fluoro-1H-indol-5-yl)oxy)-2-fluorobenzonitrile

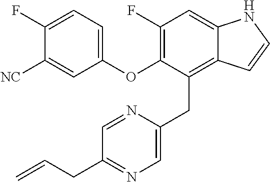 [SMILES: C=CCc1cnc(Cc2c(Oc3ccc(F)c(C#N)c3)c(F)cc3[nH]ccc23)cn1]

To a stirred solution of 5-((4-((5-Chloropyrazin-2-yl)methyl)-6-fluoro-1H-indol-5-yl)oxy)-2-fluorobenzonitrile (Intermediate 109A, 2.7 g, 6.80 mmol) and bis(tri-t-butylphosphine) palladium(0) (348 mg, 0.680 mmol) in 1,4-dioxane (60 mL) was added allyltri-n-butyltin (11 mL, 34.0 mmol) at room temperature. The mixture was stirred at 120° C. overnight, then concentrated. The residue was purified by silica gel column chromatograph to afford the title compound (1.4 g, 3.10 mmol, 51%) as a white solid. MS (ESI): 403 m/z [M+H]⁺, retention time: 1.80 minutes, purity: 89% (254 nm) (LC-MS Method 28).

Intermediate 109A: 5-((4-((5-Chloropyrazin-2-yl)methyl)-6-fluoro-1H-indol-5-yl)oxy)-2-fluorobenzonitrile

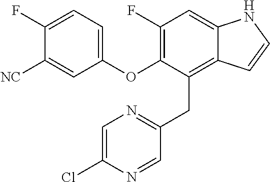 [SMILES: N#Cc1cc(Oc2c(F)cc3[nH]ccc3c2Cc2cnc(Cl)cn2)ccc1F]

To a stirred solution of 2-(bromomethyl)-5-chloropyrazine (6.50 g, 18.8 mmol) in water (20 mL) and 1,4-dioxane (100 mL) was added 2-fluoro-5-((6-fluoro-4-(4,4,5,5-tetramethyl-1,3,2-dioxaborolan-2-yl)-1H-indol-5-yl)oxy)benzonitrile (Intermediate 91, 5.96 g, 15.0 mmol), potassium carbonate (5.2 g, 37.6 mmol) and [1,1′-Bis(diphenylphosphino)ferrocene]dichloropalladium (688 mg, 0.940 mmol). The mixture was stirred at 110° C. for 16 hours, then diluted with water (100 mL) and extracted with ethyl acetate (3×100 mL). The combined organic extracts were washed with brine, dried over sodium sulfate, filtered, and concentrated. The residue was purified by automated flash chromatography (120 g silica gel column, eluting with 0-20% ethyl acetate in petroleum ether) to afford the title compound (2.7 g, 6.80 mmol, 43%) as a yellow solid. MS (ESI): 397 m/z [M+H]⁺, retention time: 1.83 minutes, purity: >99% (254 nm) (LC-MS Method 28).

Intermediate 110. 2-Fluoro-5-((6-fluoro-4-((4-(2-methylpent-4-en-2-yl)-1H-pyrazol-1-yl)methyl)-1-(phenylsulfonyl)-1H-indol-5-yl)thio)benzonitrile

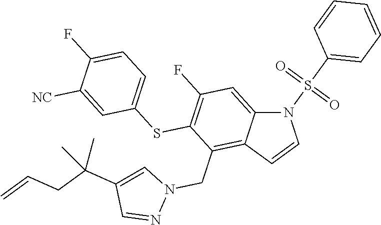 [SMILES: C=CCC(C)(C)c1cnn(Cc2c(Sc3ccc(F)c(C#N)c3)c(F)cc3c2ccn3S(=O)(=O)c2ccccc2)c1]

To a stirred solution of 5-((4-(bromomethyl)-6-fluoro-1-(phenylsulfonyl)-1H-indol-5-yl)thio)-2-fluorobenzonitrile (Intermediate 8-1, 3.3 g, 6.35 mmol) and 4-(1,1-dimethylbut-3-enyl)-1H-pyrazole (Intermediate 79, 954 mg, 1.92 mmol) in N,N-dimethylformamide (40 mL) was added cesium carbonate (4.1 g, 12.7 mmol). The resulting mixture was stirred at room temperature for 6 hours, then diluted with water and extracted with ethyl acetate (100 mL). The combined organic phases were washed with water, dried over magnesium sulfate, filtered and concentrated. The crude material was purified by flash chromatography (40 g silica gel column, eluting with 0-30% ethyl acetate in petroleum ether) to give the title compound (2.1 g, 56%) as an oil. LC-MS: MS (ESI): 589 m/z [M+H]⁺, retention time: 2.15 minutes, purity: 85% (254 nm) (LC-MS Method 5).

Intermediate 111. (R)-8-((Tert-butyldimethylsilyl)oxy)-2-(3-iodophenyl)-2,7,7-trimethyloctanoic acid

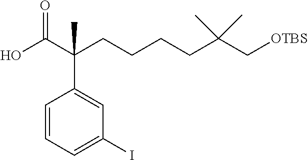 [SMILES: CC(C)(CCCC[C@@](C)(C(=O)O)c1cccc(I)c1)CO[Si](C)(C)C(C)(C)C]

To a suspension of (S)-5-Amino-5-carboxypentan-1-aminium 8-((tert-butyldimethylsilyl)oxy)-2-(3-iodophenyl)-2,7,7-trimethyloctanoate (12.4 g, 18.7 mmol) in ethyl acetate (60 mL) was added 1N hydrochloric acid (37.4 mmol, 37.4 mL). The mixture was stirred at room temperature for 2.0 hours, then diluted with water (100 mL), and extracted with ethyl acetate (3×100 mL). The combined organic layer was washed with brine, dried over sodium sulfate and concentrated. The residue was purified by flash chromatography (120 g silica gel column, eluting with 0-35% ethyl acetate in petroleum) to give the title compound (9 g, 17.5 mmol, 94%) as a light-yellow oil. LC-MS: MS (ESI): 519 m/z [M+H]⁺, retention time: 1.89 minutes, purity: 81% (254 nm) (LC-MS Method 24).

Intermediate 111A. (S)-5-Amino-5-carboxypentan-1-aminium 8-((tert-butyldimethylsilyl)oxy)-2-(3-iodophenyl)-2,7,7-trimethyloctanoate

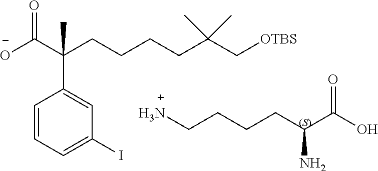

To a stirred solution of (2S)-2,6-diaminohexanoic acid (9.47 g, 64.9 mmol) in ethanol/water (v/v=9/1, 320 mL) was add a solution of 8-((tert-butyldimethylsilyl)oxy)-2-(3-iodophenyl)-2,7,7-trimethyloctanoic acid (32.0 g, 61.8 mmol) in ethanol/water (v/v=9/1, 320 mL). The mixture was stirred at 60° C. for 1 hour, cooled to room temperature (Ca. 20° C.) over 3 hours, and stirred at room temperature overnight. The precipitate was collected by filtration, rinsed with ethanol/water (v/v=9/1, 32 mL), and dried at 40° C. under vacuum for 2 hours to give the title compound (12.4 g, 18.7 mmol, 30.0%) as a white solid. LC-MS: MS (ESI): 519 m/z [M+H]⁺, retention time: 2.15 minutes, purity: 85% (254 nm) (LC-MS Method 2).

Intermediate 112. 1-Bromo-3-(1-bromo-6,6-dimethyloct-7-yn-1-yl)benzene

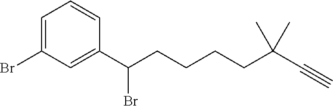 [SMILES: C#CC(C)(C)CCCCC(Br)c1cccc(Br)c1]

To a stirred solution of oxalyl chloride (14.76 g, 116.36 mmol) in dichloromethane (300 mL) was added dimethyl sulfoxide (22.72 g, 290.9 mmol) in 50 mL of dichloromethane at −78° C. The mixture was stirred at −78° C. for 3 minutes, then treated with a solution of 7-bromo-7-(3-bromophenyl)-2,2-dimethylheptan-1-ol (Intermediate 112C, 22 g, 58.18 mmol) in 75 mL of dichloromethane. After stirring for 20 minutes at this temperature, triethylamine (58.87 g, 581.8 mmol) was added. The reaction mixture was stirred for 10 minutes, quenched with water, and extracted with dichloromethane. The combined organic phase was washed with brine, dried over sodium sulfate, filtered, and concentrated to afford 7-bromo-7-(3-bromophenyl)-2,2-dimethylheptanal (8.4 g), which was used for the next step without further purification.

To a stirred solution of 1-diazo-1-dimethoxyphosphoryl-propan-2-one (6.64 g, 34.6 mmol) in methanol (250 mL) was added potassium carbonate (9.55 g, 69.1 mmol). The mixture was stirred at 0° C. for 5 minutes, then treated with 7-bromo-7-(3-bromophenyl)-2,2-dimethylheptanal (10.00 g, 26.6 mmol). The reaction was stirred at 0° C. for 2 hours, quenched with saturated ammonium chloride (100 mL), and extracted with ethyl acetate (2×100 mL). The combined organic phase was washed with saturated sodium bicarbonate, brine, dried over sodium sulfate, filtered, and concentrated. The residue was purified by silica gel column chromatography (eluted with 100% petroleum ether) to afford the title compound (6.5 g, 66%) as a pale yellow oil. LC-MS: MS (ESI): 373 m/z [M+H]⁺, retention time: 2.15 minutes, purity: 85% (254 nm) (LC-MS Method 2).

Intermediate 112A. Methyl 7-(3-bromophenyl)-2,2-dimethylheptanoate

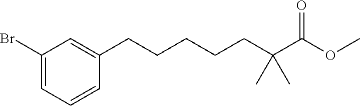 [SMILES: COC(=O)C(C)(C)CCCCCc1cccc(Br)c1]

To a stirred solution of methyl isobutyrate (7.65 g, 75.0 mmol) in tetrahydrofuran (153 mL) was added lithium diisopropylamide (2M in tetrahydrofuran, 75 mmol, 37.5 mL) at −78° C. The mixture was stirred for 30 minutes, then treated with 1-bromo-3-(5-bromopentyl)benzene (15.3 g, 50.0 mmol). The mixture was slowly warmed to room temperature and stirred for 16 hours, quenched with saturated ammonium chloride (150 mL), and extracted with ethyl acetate (3×100 mL). The combined organic phase was washed with brine, dried over sodium sulfate, filtered, and concentrated. The residue was purified by silica gel flash column chromatography (eluted with 1-2% ethyl acetate in petroleum) to afford the title compound (11.52 g, 70%). LC-MS: MS (ESI): 327, 329 m/z [M+H]⁺, retention time: 2.27 minutes, purity: 93% (214 nm) (LC-MS Method 9).

Intermediate 112B. 7-(3-Bromophenyl)-2,2-dimethylheptan-1-ol

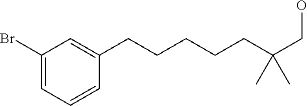 [SMILES: CC(C)(CO)CCCCCc1cccc(Br)c1]

To a stirred solution of Intermediate 112A (58.24 g, 177.96 mmol) in tetrahydrofuran (600 mL) was added lithium aluminum hydride (2.5M in tetrahydrofuran, 221 mmol, 88.3 mL) at −40° C. The solution was stirred at −20° C. for 1 hour, then quenched with ammonium chloride (300 mL), and extracted with ethyl acetate (3×150 mL). The combined organic phase was washed with brine, dried over sodium sulfate, filtered, and concentrated. The residue was purified by column chromatography (eluted with 20% to 50% dichloromethane in petroleum ether) to afford the title compound (9.34 g, 93%) as a colorless oil. LC-MS: MS (ESI): 299, 301 m/z [M+H]⁺, retention time: 2.10 minutes, purity: 89% (214 nm) (LC-MS Method 9).

Intermediate 112C. 7-Bromo-7-(3-bromophenyl)-2,2-dimethylheptan-1-ol

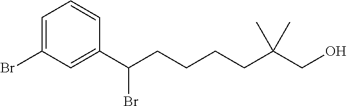 [SMILES: CC(C)(CO)CCCCC(Br)c1cccc(Br)c1]

To a stirred solution of Intermediate 112B (44 g, 147.03 mmol) in carbon tetrachloride (650 mL) was added azodiisobutyronitrile (2.41 g, 14.7 mmol) and N-bromosuccinimide (31.4 g, 176 mmol). The mixture was stirred at 80° C. for 1 hour, cooled to room temperature, quenched with saturated sodium bicarbonate (600 mL), and extracted with dichloromethane (3×200 mL). The combined organic phase was washed with brine, dried over sodium sulfate, filtered, and concentrated. The residue was purified by silica gel column chromatography (eluted with 7-12% ethyl acetate in petroleum ether) to afford the title compound (53.41 g, 96%). LC-MS: MS (ESI): 379 m/z [M+H]⁺, retention time: 2.11 minutes, purity: 84% (214 nm) (LC-MS Method 26).

Intermediate 113. Methyl 4-((6-fluoro-4-(hydroxymethyl)-1-tosyl-1H-indol-5-yl)oxy)pyridine-2-carbimidothioate

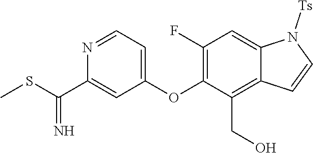 [SMILES: CSC(=N)c1cc(Oc2c(F)cc3c(ccn3S(=O)(=O)c3ccc(C)cc3)c2CO)ccn1]

To a stirred solution of 4-((6-fluoro-4-(hydroxymethyl)-1-tosyl-1H-indol-5-yl)oxy)pyridine-2-carbothioamide (Intermediate 56-2, 1.00 g, 2.12 mmol) in acetone (15 mL) were added methyl iodide (0.64 mL, 10.3 mmol) and sodium bicarbonate (863 mg, 10.3 mmol).

The reaction mixture was stirred at room temperature overnight, then quenched with water (50 mL), and extracted with ethyl acetate (3×50 mL). The combined organic layers were washed with brine, dried over sodium sulfate and concentrated to give the title compound (970 mg, 1.58 mmol, 77.00%) as a solid. LC-MS: MS (ESI): 486 m/z [M+H]⁺, retention time: 1.63 minutes, purity: 83% (214 nm) (LC-MS Method 26).

The following intermediate was prepared based on the procedures described for Intermediate 113.

| Inter. | | | MS m/z |
|---|---|---|---|
| No. | Structure | Name | [M + H]⁺ |
| 113-1 | 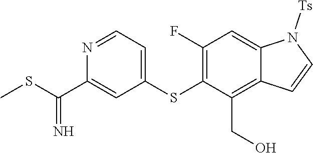 [SMILES: CSC(=N)c1cc(Sc2c(F)cc3c(ccn3S(=O)(=O)c3ccc(C)cc3)c2CO)ccn1] | methyl 4-((6-fluoro-4-(hydroxymethyl)-1-tosyl-1H- indol-5-yl)thio)pyridine-2-carbimidothioate | 502 |

Intermediate 114. 4-((4-Bromo-6-fluoro-1H-indol-5-yl)thio)picolinonitrile

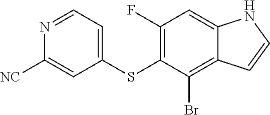 [SMILES: N#Cc1cc(Sc2c(F)cc3[nH]ccc3c2Br)ccn1]

To a stirred solution of 4-(2-bromo-6-fluoro-3-methyl-4-nitro-phenyl)sulfanylpyridine-2-carbonitrile (Intermediate 114B, 70.00 g, 190 mmol) in dimethylformamide (350 mL) was added N,N-dimethylformamide dimethyl acetal (76 mL, 570 mmol). The mixture was stirred at 100° C. for 4 hours and concentrated. The reside, a brown solid (75 g, (E)-4-((2-bromo-3-(2-(dimethylamino)vinyl)-6-fluoro-4-nitrophenyl)thio)picolinonitrile), was used for next step without further purification. LC-MS: MS (ESI): 396 m/z [M+H]⁺, retention time: 1.76 minutes, purity: 37% (254 nm) (LC-MS Method 26).

To a stirred solution of the above brown reside (75.0 g, 177 mmol) in methanol (200 mL) and tetrahydrofuran (200 mL) was added stannous chloride dihydrate (79.97 g, 354 mmol). The mixture was stirred at room temperature for 2 hours, diluted with 1000 mL of water and 1000 mL of ethyl acetate, alkalified with 1N sodium hydroxide solution to pH ˜8. The mixture was filtered, the filter cake was washed with ethyl acetate, and the filtrate was extracted with ethyl acetate (3×1000 mL). The combined organic phase was washed with brine, dried over sodium sulfate, and concentrated to give 4-(4-bromo-6-fluoro-1-hydroxy-indol-5-yl)sulfanylpyridine-2-carbonitrile (61.00 g, 167 mmol, 94.53% yield) as a brown solid, which was used for next step without further purification. LC-MS: MS (ESI): 364 m/z [M+H]⁺, retention time: 1.78 minutes, purity: 58% (254 nm) (LC-MS Method 3).

To a stirred solution of 2-bromo-1-phenyl-ethanone (33.3 g, 167 mmol) in tetrahydrofuran (150 mL) and methanol (150 mL) was added 4-(4-bromo-6-fluoro-1-hydroxy-indol-5-yl)sulfanylpyridine-2-carbonitrile (the above brown solid, 61.00 g, 167 mmol) and triethylamine (47 mL, 335 mmol). The mixture was stirred at room temperature for 3 hours, diluted with 1000 mL of water, extracted with ethyl acetate (3×1000 mL). The combined organic phase was washed with brine, dried over sodium sulfate and concentrated. The residue was purified by silica gel column chromatography (eluting with 0-50% of ethyl acetate in petroleum ether) to give 4-[(4-bromo-6-fluoro-1H-indol-5-yl)sulfanyl]pyridine-2-carbonitrile (25.00 g, 71.8 mmol, 43% 3 steps) as a yellow solid. ¹H NMR (400 MHz, CDCl₃) δ 8.43 (dd, J=4.8, 0.8 Hz, 1H), 7.3-7.37 (m, 1H), 7.29 (dd, J=8.4, 0.8 Hz, 1H), 7.16-7.14 (m, 2H), 6.71-6.69 (m, 1H).

The following intermediate was prepared based on the procedures described for Intermediate 114.

| Inter. No. | Structure | Name | MS m/z [M + H]⁺ |
|---|---|---|---|
| 114-1 | 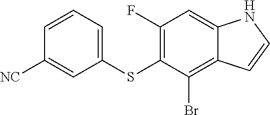 [SMILES: N#Cc1cccc(Sc2c(F)cc3[nH]ccc3c2Br)c1] | 3-((4-bromo-6-fluoro-1H-indol-5-yl)thio)benzonitrile | 347, 349 |

Intermediate 114A. 2-Cyanopyridin-4-yl carbamimidothioate hydrochloride

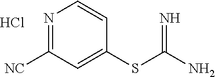 [SMILES: Cl ; N#Cc1cc(SC(=N)N)ccn1]

To a suspension of 4-chloropyridine-2-carbonitrile (100 g, 722 mmol) in ethanol (300 mL) was added thiourea (57.69 g, 758 mmol). The mixture was refluxed for 2 and half hours, cooled to room temperature. The formed precipitate was collected by filtration and rinsed with ethanol (2×200 mL), dried to give the title compound (134 g, 593 mmol, 82%) as a green solid. LC-MS: MS (ESI): 179 m/z [M+H]⁺, retention time: 0.35 minutes, purity: >90% (214 nm) (LC-MS Method 3).

Intermediate 114B. 4-((2-Bromo-6-fluoro-3-methyl-4-nitrophenyl)thio)picolinonitrile

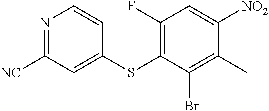 [SMILES: Cc1c([N+](=O)[O-])cc(F)c(Sc2ccnc(C#N)c2)c1Br]

To a stirred solution of 2-(2-cyano-4-pyridyl)isothiourea hydrochloride (123 g, 573 mmol) in N,N-dimethylformamide (720 mL) was added sodium hydroxide (45.84 g, 1146 mmol) and water (574 g, 32 mol), the reaction mixture was stirred at room temperature for 15 minutes, then treated with 3-bromo-1,2-difluoro-4-methyl-5-nitro-benzene (151.61 g, 602 mmol), The mixture was stirred at room temperature for 2 hours, then filtered. The filter cake was rinsed with water (3×1 L), then dissolved in ethyl acetate (1 L). This solution was washed with water, brine, dried over sodium sulfate, filtered, and concentrated. The residue was then slurred with petroleum ether (1 L) for 1 hour. The solid was collected by filtration to afford the title compound (200 g, 489 mmol, 85%). LC-MS: MS (ESI): 368, 370 m/z [M+H]⁺, retention time: 1.76 minutes, purity: 91% (254 nm) (LC-MS Method 33).

Intermediate 115. 1-(3-((6-Fluoro-4-(((4-methoxybenzyl)oxy)methyl)-1-tosyl-1H-indol-5-yl)thio)phenyl)ethan-1-one

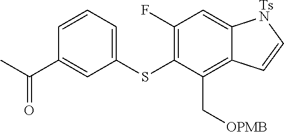

To a stirred solution of 1-(3-((4-bromo-6-fluoro-1-tosyl-1H-indol-5-yl)thio)phenyl)ethan-1-one (Intermediate 73B-1, 620 mg, 1.20 mmol) in 1,4-dioxane (12 mL) and water (2.5 mL) were added potassium trifluoro-[methoxy-(4-methoxyphenyl) methyl]boranide (463 mg, 1.79 mmol), cesium carbonate (974 mg, 2.99 mmol) and cataCXium-Pd-G3 (87 mg, 0.120 mmol). The mixture was stirred at 80° C. for 3 hours, cooled to room temperature, diluted with water (15 mL), and extracted with ethyl acetate (3×15 mL). The combined organic layer was washed with brine (20 mL), dried over sodium sulfate, filtered, and concentrated. The residue was purified by automated silica gel flash chromatography (40 g silica gel column, eluting with 20-100% dichloromethane in petroleum ether) to give the title compound (450 mg, 0.725 mmol, 61%) as a brown oil. LC-MS: MS (ESI): 612 m/z [M+Na]⁺, retention time: 2.18 minutes, purity: 95% (254 nm) (LC-MS Method 32).

The following intermediate was prepared based on the procedures described for Intermediate 115.

| Inter. | | | MS m/z |
|---|---|---|---|
| No. | Structure | Name | [M + H]⁺ |
| 115-1 | 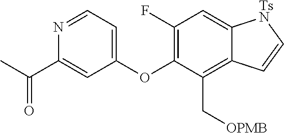 | 1-(4-((6-fluoro-4-(((4-methoxybenzyl)oxy)methyl)-1-tosyl-1H- indol-5-yl)oxy)pyridin-2-yl)ethan-1-one | 575 |

Intermediate 116. (5-((3-(1H-Pyrazol-3-yl)phenyl)thio)-6-fluoro-1-tosyl-1H-indol-4-yl)methanol

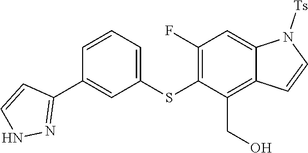 [SMILES: Cc1ccc(S(=O)(=O)n2ccc3c(CO)c(Sc4cccc(-c5cc[nH]n5)c4)c(F)cc32)cc1]

To a stirred solution of 5-((3-(1H-pyrazol-3-yl)phenyl)thio)-6-fluoro-4-(((4-methoxybenzyl)oxy)methyl)-1-tosyl-1H-indole (Intermediate 73-5, 1.30 g, 2.05 mmol) in dichloromethane (12 mL) was added trifluoroacetic acid (9.0 mL). The mixture was stirred at room temperature for 1 hour and concentrated. The residue was purified by automated flash chromatography (80 g silica gel column, eluting with 0-5% methanol in dichloromethane) to give the title compound (1.3 g, 1.16 mmol, 56.40%) as a yellow oil. LC-MS: MS (ESI): 494 m/z [M+H]⁺, retention time: 2.00 minutes, purity: 44% (254 nm) (LC-MS Method 26).

The following intermediate was prepared based on the procedures described for Intermediate 116.

| Inter. | | | MS m/z |
|---|---|---|---|
| No. | Structure | Name | [M + H]⁺ |
| 116-1 | 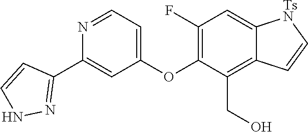 [SMILES: Cc1ccc(S(=O)(=O)n2ccc3c(CO)c(Oc4ccnc(-c5cc[nH]n5)c4)c(F)cc32)cc1] | (5-((2-(1H-pyrazol-3-yl)pyridin-4-yl)oxy)-6-fluoro-1-tosyl-1H- indol-4-yl)methanol | 479 |

Intermediate 117. Methyl 4-((6-fluoro-4-(hydroxymethyl)-1H-indol-5-yl)oxy)pyridine-2-carbimidothioate

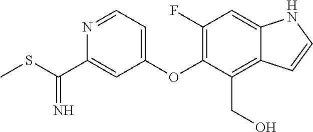 [SMILES: CSC(=N)c1cc(Oc2c(F)cc3[nH]ccc3c2CO)ccn1]

To a stirred solution of 4-((6-fluoro-4-(hydroxymethyl)-1H-indol-5-yl)oxy)pyridine-2-carbothioamide (Intermediate 56-4, 8.8 g, 27.7 mmol) in acetone (20 mL) were added methyl iodide (9.0 mL, 139 mmol) and sodium bicarbonate (11.6 g, 138.5 mmol). The mixture was stirred at room temperature overnight, quenched with water (50 mL), and extracted with ethyl acetate (3×50 mL). The combined organic extracts were washed with brine (50 mL), dried over anhydrous sodium sulfate, filtered, and concentrated. The residue was purified by automated flash chromatography (40 g silica gel column, eluting with 0-70% ethyl acetate in petroleum ether) to afford the title product (8.6 g, 25.9 mmol, 93%) as a white solid. LC-MS: MS (ESI): 332 m/z [M+H]⁺, retention time: 1.83 minutes, purity: 80% (214 nm) (LC-MS Method 2).

The following intermediate was prepared based on the procedures described for Intermediate 117.

| Inter. | | | |
|---|---|---|---|
| No. | Structure | Name | MS m/z [M + H]⁺ |
| 117-1 | 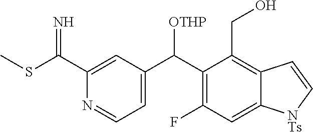 | methyl 4-((6-fluoro-4-(hydroxymethyl)-1-tosyl- 1H-indol-5-yl)((tetrahydro-2H-pyran-2- yl)oxy)methyl)pyridine-2-carbimidothioate | 584 m/z [M + H]⁺; RT: 1.09 + 1.10 minute (LC-MS method 35) |

Intermediate 118A. 4-((4-Bromo-6-fluoro-1-(triisopropylsilyl)-1H-indol-5-yl)(hydroxy)methyl)picolinonitrile

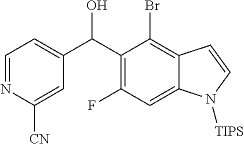

To a stirred solution of 4-bromo-6-fluoro-1-(triisopropylsilyl)-1H-indole (35.00 g, 94.5 mmol) in tetrahydrofuran (700 mL) at −78° C. was added lithium diisopropylamide (57 mL, 113 mmol). The mixture was stirred at −78° C. for 1 hour, then treated with 4-formylpyridine-2-carbonitrile (13.73 g, 104 mmol), and stirred at −78° C. for another 2 hours. The mixture was quenched with saturated ammonium chloride (200 mL) and extracted with ethyl acetate (3×150 mL). The combined organic extracts were washed with brine, dried over sodium sulfate, filtered, and concentrated. The residue was purified by automated flash chromatography (330 g silica gel column, eluting with 0-40% ethyl acetate in petroleum ether) to give the title compound (33.00 g, 65.7 mmol, 69.50%) as a solid. LC-MS: MS (ESI): 502, 504 m/z [M+H]⁺, retention time: 1.49 minutes, purity: >99% (214 nm) (LC-MS Method 35).

Intermediate 118B. 4-((4-Bromo-6-fluoro-1-(triisopropylsilyl)-1H-indol-5-yl)((tetrahydro-2H-pyran-2-yl)oxy)methyl)picolinonitrile

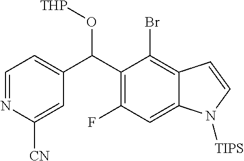

To a stirred solution of 4-((4-bromo-6-fluoro-1-(triisopropylsilyl)-1H-indol-5-yl)(hydroxy)methyl)picolinonitrile (Intermediate 118A, 5.00 g, 9.95 mmol) in dichloromethane (50 mL) was added 3,4-dihydro-2H-pyran (1.8 mL, 19.9 mmol) and pyridinium p-toluenesulfonate (250 mg, 0.995 mmol). The mixture was stirred at room temperature overnight.

The mixture was washed with brine (50 mL), dried over magnesium sulfate, filtered, and concentrated. The residue was purified by automated flash chromatography (120 g silica gel column, eluting with 0-40% ethyl acetate in petroleum) to give the title compound (3.90 g, 67%) as a solid. LC-MS: MS (ESI): 586, 588 m/z [M+H]⁺, retention time: 2.70+2.80 minutes, purity: 48+42% (214 nm) (LC-MS Method 26).

Intermediate 118C. 4-((6-Fluoro-1-(triisopropylsilyl)-4-vinyl-1H-indol-5-yl)((tetrahydro-2H-pyran-2-yl)oxy)methyl)picolinonitrile

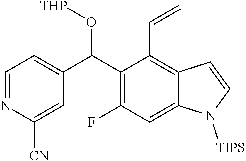

To a stirred solution of 4-((4-bromo-6-fluoro-1-(triisopropylsilyl)-1H-indol-5-yl)((tetrahydro-2H-pyran-2-yl)oxy)methyl)picolinonitrile (Intermediate 118B, 3.90 g, 6.65 mmol) and 4,4,5,5-tetramethyl-2-vinyl-1,3,2-dioxaborolane (1.23 g, 7.98 mmol) in dioxane/water(10:1) (55 mL) was added tris(dibenzylideneacetone)dipalladium(0) (0.609 g, 0.66 mmol), potassium carbonate (2.76 g, 19.9 mmol) and tri-tert-butylphosphine tetrafluoroborate (387 mg, 1.33 mmol). The suspension was degassed and purged with N₂ for three times and stirred at 50° C. for 1 hour under nitrogen. The reaction mixture was cooled to room temperature, diluted with water, and extracted with ethyl acetate (2×100 mL). The combined organic layers were washed with brine, dried over sodium sulphate, filtered, and concentrated under reduced pressure. The residue was purified by automated flash chromatography (120 g silica gel column, eluting with 0-20% ethyl acetate in petroleum ether) to give the title compound (3.55 g, 100%) as a solid. LC-MS: MS (ESI): 534 m/z [M+H]⁺, retention time: 2.64+2.75 minutes, purity: 42+32% (214 nm) (LC-MS Method 26).

Intermediate 118D. 4-((6-Fluoro-4-vinyl-1H-indol-5-yl)((tetrahydro-2H-pyran-2-yl)oxy)methyl)picolinonitrile

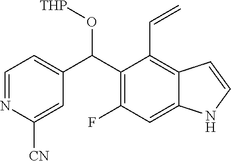

To a stirred solution of 4-((6-fluoro-1-(triisopropylsilyl)-4-vinyl-1H-indol-5-yl)((tetrahydro-2H-pyran-2-yl)oxy)methyl)picolinonitrile (Intermediate 118C, 3.55 g, 6.65 mmol) in tetrahydrofuran (40 mL) was added tera-n-butylammonium fluoride (3.69 g, 13.3 mmol). The reaction mixture was stirred at room temperature for 1 hour, then quenched with water (50 mL), and extracted with ethyl acetate (2×20 mL). The combined organic extracts were washed with brine, dried over sodium sulfate, filtered, and concentrated. The residue was purified by automated flash chromatography (40 g silica gel column, eluting with 0-50% ethyl acetate in petroleum ether) to give the title compound (1.88 g, 75%) as a solid. LC-MS: MS (ESI): 378 m/z [M+H]⁺, retention time: 1.89+1.93 minutes, purity: 46+42% (214 nm) (LC-MS Method 26).

### Preparation of Exemplified Compounds

Example 1. Compound 1. 3-[3-(24,30-difluoro-6-methyl-26-oxa-3,12,13,14,21,33-hexazahexacyclo[25.3.1.12,5.111,14.017,25.018,22]tritriaconta-1(31),2,4,11(32),12,17,19,22,24,27,29-undecaen-6-yl)phenyl]propanoic acid

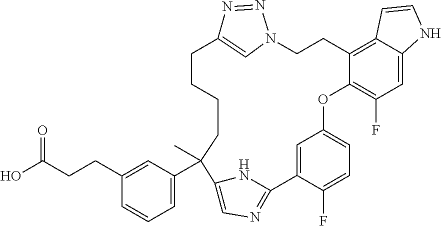 [SMILES: CC1(c2cccc(CCC(=O)O)c2)CCCCc2cn(nn2)CCc2c(c(F)cc3[nH]ccc23)Oc2ccc(F)c(c2)-c2ncc1[nH]2]

4-(2-Azidoethyl)-5-(3-(5-(2-(3-bromophenyl)oct-7-yn-2-yl)-1H-imidazol-2-yl)-4-fluorophenoxy)-6-fluoro-1H-indole

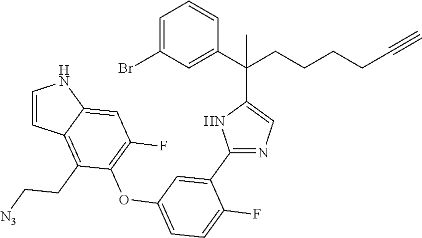 [SMILES: C#CCCCCC(C)(c1cccc(Br)c1)c1cnc(-c2cc(Oc3c(F)cc4[nH]ccc4c3CCN=[N+]=[N-])ccc2F)[nH]1]

Step A: To a stirred solution of 5-((4-(2-azidoethyl)-6-fluoro-1H-indol-5-yl)oxy)-2-fluorobenzimidamide (Intermediate 6, 2.0 g, 4.05 mmol) in dimethylformamide (40 mL) was added 1-bromo-3-(3-bromophenyl)-3-methylnon-8-yn-2-one (Intermediate 2, 1.6 g, 4.05 mmol) and potassium carbonate (1.7 g, 20.25 mmol). The mixture was heated at 70° C. for 1 hour, then cooled to room temperature and partitioned between water (50 mL) and ethyl acetate (50 mL). The separated organic phase, combined with two additional ethyl acetate extracts (2×50 mL), was washed with water, brine, dried over sodium sulfate, and concentrated. The residue was purified by silica gel column chromatography (petroleum ether/ethyl acetate=2:1) to give the title compound (480 mg, 18%) as a yellow solid. MS: 643, 645 m/z [M+H]⁺.

6-(3-Bromophenyl)-24,30-difluoro-6-methyl-26-oxa-3,12,13,14,21,33-hexazahexacyclo-[25.3.1.12,5.111,14.017,25.018,22]tritriaconta-1(31),2,4,11(32),12,17,19,22,24,27,29-undecaene

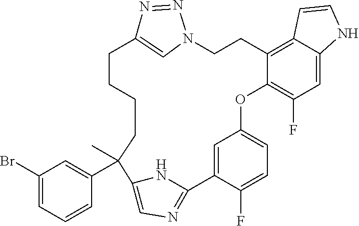 [SMILES: CC1(c2cccc(Br)c2)CCCCc2cn(nn2)CCc2c(c(F)cc3[nH]ccc23)Oc2ccc(F)c(c2)-c2ncc1[nH]2]

Step B: To a stirred solution of Step A product (190 mg, 0.30 mmol) in t-butyl alcohol (30 mL) and H₂O (30 mL) was added copper (II) sulfate (48 mg, 0.3 mmol) and sodium L-ascorbate (119 mg, 0.6 mmol). The mixture was stirred at room temperature for 16 hours and diluted with ethyl acetate (50 mL). The solution was washed with water, brine, dried over sodium sulfate, and concentrated. The residue was purified by silica gel column chromatography (petroleum ether/ethyl acetate=1:1) to give the title compound (20 mg, 8%) as a yellow solid. MS: 643, 645 m/z [M+H]⁺.

Ethyl (E)-3-[3-(24,30-difluoro-6-methyl-26-oxa-3,12,13,14,21,33-hexazahexacyclo-[25.3.1.12,5.111,14.017,25.018,22]tritriaconta-1(31),2,4,11(32),12,17,19,22,24,27,29-undecaen-6-yl)phenyl]prop-2-enoate

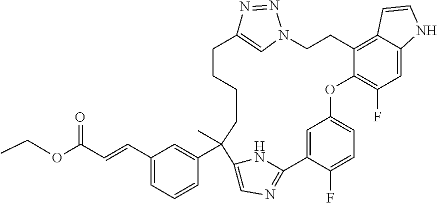 [SMILES: CCOC(=O)/C=C/c1cccc(C2(C)CCCCc3cn(nn3)CCc3c(c(F)cc4[nH]ccc34)Oc3ccc(F)c(c3)-c3ncc2[nH]3)c1]

Step C: In a glove box, to a reaction tube was added Step B product (20 mg, 29 μmol), ethyl acrylate (20 mL, 183 μmol), tri(o-tolyl)phosphine (3 mg, 10 μmol), palladium(II) acetate (1 mg, 4.7 μmol), dimethylformamide (2 mL) and triethylamine (22 μL, 155 μmol). The reaction was stirred at 125° C. overnight. The mixture was cooled to room temperature and diluted with ethyl acetate (100 mL). The solution was washed with water, brine, dried with sodium sulfate, and concentrated. The residue was purified by prep-thin layer chromatography (petroleum ether/ethyl acetate=1:1) to give the title compound (12 mg, 63%) as a yellow solid. MS (ESI): 663 m/z [M+H]⁺, retention time: 1.79 minutes, purity: 92% (254 nm) (LC-MS method 2).

Ethyl 3-[3-(24,30-difluoro-6-methyl-26-oxa-3,12,13,14,21,33-hexazahexacyclo-[25.3.1.12,5.111,14.017,25.018,22]tritriaconta-1(31),2,4,11(32),12,17,19,22,24,27,29-undecaen-6-yl)phenyl]propanoate

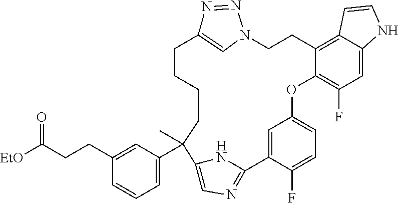 [SMILES: CCOC(=O)CCc1cccc(C2(C)CCCCc3cn(nn3)CCc3c(c(F)cc4[nH]ccc34)Oc3ccc(F)c(c3)-c3ncc2[nH]3)c1]

Step D: To a stirred solution of Step C product (40 mg, 44 μmol) in tetrahydrofuran (10 mL) was added 10% palladium on carbon (12 mg). The mixture was stirred at room temperature under a hydrogen balloon overnight, then filtered through a pad of Celite and rinsed with tetrahydrofuran (20 mL). The filtrate was concentrated. The residue was purified by prep-TLC (petroleum ether/ethyl acetate=1:1) to give the title compound (8 mg, 66%) as a yellow oil. MS (ESI): 665 m/z [M+H]⁺, retention time: 1.75 minutes, purity: 94% (254 nm) (LC-MS method 2).

Compound 1: 3-[3-(24,30-Difluoro-6-methyl-26-oxa-3,12,13,14,21,33-hexazahexacyclo-[25.3.1.12,5.111,14.017,25.018,22]tritriaconta-1(31),2,4,11(32),12,17,19,22,24,27,29-undecaen-6-yl)phenyl]propanoic acid

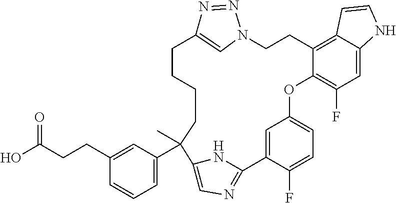 [SMILES: CC1(c2cccc(CCC(=O)O)c2)CCCCc2cn(nn2)CCc2c(c(F)cc3[nH]ccc23)Oc2ccc(F)c(c2)-c2ncc1[nH]2]

Step E: To a stirred solution of Step D product (8 mg, 12 μmol) in methanol (2 mL) and tetrahydrofuran (6 mL) was added lithium hydroxide monohydrate (1 M in water, 2 mL). The mixture was stirred at room temperature for 4 hours, then acidified with 1 N hydrochloric acid to pH ˜4 and diluted with ethyl acetate (50 mL). The solution was washed with brine, dried over sodium sulfate, and concentrated. The residue was purified by prep-thin layer chromatography (dichloromethane: methanol=10:1) to give the title compound (3.9 mg, 50%) as a solid. MS (ESI): 637 m/z [M+H]⁺, retention time: 1.63 minutes, purity: >99% (214 nm) (LC-MS method 3). ¹H NMR (400 MHz, CD₃OD) δ 7.49 (dd, J=5.9, 3.1 Hz, 1H), 7.20-7.10 (m, 2H), 7.08-6.98 (m, 4H), 6.95-6.88 (m, 3H), 6.85 (s, 1H), 5.77 (d, J=3.0 Hz, 1H), 4.47-4.34 (m, 1H), 3.89-3.75 (m, 1H), 3.33-3.23 (m, 2H), 2.75 (t, J=7.7 Hz, 2H), 2.47-2.38 (m, 4H), 2.27-2.18 (m, 1H), 1.99-1.88 (m, 1H), 1.76-1.67 (m, 1H), 1.59-1.40 (m, 4H), 1.37-1.23 (m, 2H) ppm.

Example 2. Compound 2. 3-[3-(23,29-difluoro-6-methyl-25-oxa-3,12,13,14,20,32-hexazahexacyclo[24.3.1.12,5.111,14.016,24.017,21]dotriaconta-1(30),2,4,11(31),12,16,18,21,23,26,28-undecaen-6-yl)phenyl]propanoic acid

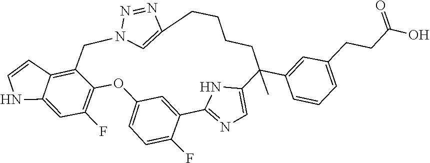 [SMILES: CC1(c2cccc(CCC(=O)O)c2)CCCCc2cn(nn2)Cc2c(c(F)cc3[nH]ccc23)Oc2ccc(F)c(c2)-c2ncc1[nH]2]

4-(Azidomethyl)-6-fluoro-5-(4-fluoro-3-(5-(2-(3-iodophenyl)oct-7-yn-2-yl)-1H-imidazol-2-yl)phenoxy)-1H-indole

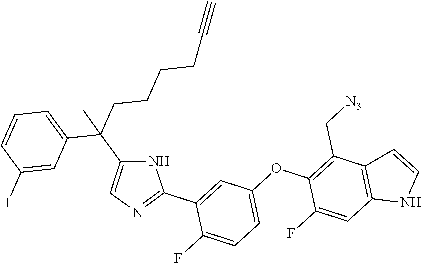 [SMILES: C#CCCCCC(C)(c1cccc(I)c1)c1cnc(-c2cc(Oc3c(F)cc4[nH]ccc4c3CN=[N+]=[N-])ccc2F)[nH]1]

Step A: To a stirred solution of 5-((4-(azidomethyl)-6-fluoro-1H-indol-5-yl)oxy)-2-fluorobenzimidamide (Intermediate 6-1, 870 mg, 2.544 mmol) in dimethylformamide (10 mL) was added 1-bromo-3-(3-iodophenyl)-3-methylnon-8-yn-2-one (Intermediate 2-51, 1.0 g, 2.314 mmol) and sodium bicarbonate (641 mg, 7.632 mmol). The reaction mixture was stirred at 60° C. for 18 hours, cooled to room temperature, and quenched with water (30 mL). The solution was extracted with ethyl acetate (3×30 mL). The combined organic layers were dried over sodium sulfate and concentrated. The residue was purified by silica gel column chromatography (petroleum ether ethyl acetate=10:0 to 2:1 to afford the title compound (670 mg, 39%). MS (ESI): 677 m/z [M+H]⁺.

23,29-difluoro-6-(3-iodophenyl)-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaene

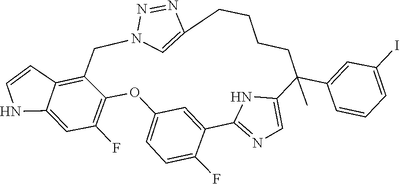 [SMILES: CC1(c2cccc(I)c2)CCCCc2cn(nn2)Cc2c(c(F)cc3[nH]ccc23)Oc2ccc(F)c(c2)-c2ncc1[nH]2]

Step B: To a solution of Step A product (670 mg, 0.991 mmol) in dichloromethane (30 mL) was added tris[(1-benzyl-1H-1,2,3-triazol-4-yl)methyl]amine (TBTA, 30 mg, 0.099 mmol) and tetrakis(acetonitrile)copper(I) tetrafluoroborate (31 mg, 0.0991 mmol). The reaction mixture was stirred at 45° C. for 18 hours under nitrogen, then cooled to room temperature, and diluted with water (10 mL). The formed solid was collected by filtration and dried to afford the title compound (300 mg, 45%). MS (ESI): 677 m/z [M+H]⁺.

Ethyl (E)-3-[3-(23,29-difluoro-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaen-6-yl)phenyl]prop-2-enoate

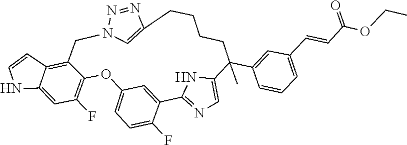 [SMILES: CCOC(=O)/C=C/c1cccc(C2(C)CCCCc3cn(nn3)Cc3c(c(F)cc4[nH]ccc34)Oc3ccc(F)c(c3)-c3ncc2[nH]3)c1]

Step C: To a stirred solution of Step B product (300 mg, 0.444 mmol) in dimethylformamide (5 mL) was added tri-(ortho-tolyl)phosphine ((40 mg, 0.133 mmol), ethyl acrylate (222 mg, 2.22 mmol), palladium acetate (10 mg, 0.0444 mmol). The reaction mixture was heated at 100° C. for 1.5 hours in a microwave reactor. The reaction mixture was diluted with water (20 mL) and extracted with ethyl acetate (3×30 mL). The combined organic layers were dried over sodium sulfate, and concentrated. The residue was purified by silica gel column chromatography (petroleum ether: ethyl acetate=10:0 to 1:1 to afford the title compound (110 mg, 38%). MS (ESI): 649 m/z [M+H]⁺.

Ethyl 3-[3-(23,29-difluoro-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaen-6-yl)phenyl]propanoate

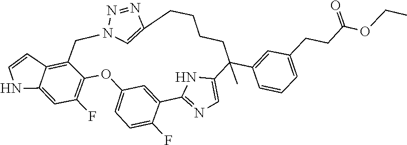 [SMILES: CCOC(=O)CCc1cccc(C2(C)CCCCc3cn(nn3)Cc3c(c(F)cc4[nH]ccc34)Oc3ccc(F)c(c3)-c3ncc2[nH]3)c1]

Step D: To a stirred solution of Step C product (110 mg, 0.225 mmol) in ethyl acetate (8 mL) was added 10% Pd/C (50% wet, 30 mg). The reaction mixture was stirred at room temperature for 18 hours under hydrogen balloon, then filtered through a pad of Celite. The filtrate was concentrated. The residue was purified by reverse phase column chromatography (water (0.1% trifluoroacetic acid): acetonitrile (0.1% trifluoroacetic acid)=60%: 40%) to afford the title compound (61 mg, 55%) as a solid. %). MS (ESI): 651 m/z [M+H]⁺.

Compound 2: 3-[3-(23,29-difluoro-6-methyl-25-oxa-3,12,13,14,20,32-hexazahexacyclo[24.3.1.12,5.111,14.016,24.017,21]dotriaconta-1(30),2,4,11(31),12,16,18,21,23,26,28-undecaen-6-yl)phenyl]propanoic acid

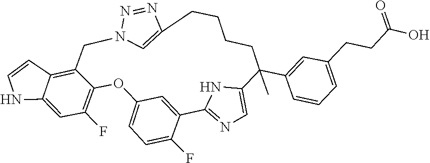 [SMILES: CC1(c2cccc(CCC(=O)O)c2)CCCCc2cn(nn2)Cc2c(c(F)cc3[nH]ccc23)Oc2ccc(F)c(c2)-c2ncc1[nH]2]

Step E: To a solution of Step D product (61 mg, 0.094 mmol) in tetrahydrofuran/methanol (4/1) (10 mL) was added 1.0 M lithium hydroxide (5 mL). The reaction mixture was stirred at room temperature for 18 hours, then adjusted pH to 6 with 1.0 M hydrochloric acid and extracted with ethyl acetate (3×20 mL). The combined organic layers were dried over sodium sulfate and concentrated. The residue was purified by reverse phase column water (0.1% trifluoroacetic acid): acetonitrile (0.1% trifluoroacetic acid)=40%: 60%) to afford the title compound (25.8 mg, 44%). MS m/z: 623.1 (M+H⁺). ¹H NMR (400 MHz, CD₃OD) δ 7.56-6.97 (m, 10H), 6.63-6.53 (m, 2H), 5.99-5.80 (m, 2H), 2.86-2.50 (m, 6H), 2.18-1.90 (m, 2H), 1.68-1.55 (m, 4H), 1.37-1.17 (m, 2H), 0.90-0.80 (m, 1H) ppm.

Example 3. Compound 3. 3-[3-(23,29-difluoro-6-methyl-25-oxa-3,12,13,14,20,32-hexazahexacyclo[24.3.1.12,5.111,14.016,24.017,21]dotriaconta-1(30),2,4,11(31),12,16,18,21,23,26,28-undecaen-6-yl)-2-fluoro-phenyl]propanoic acid

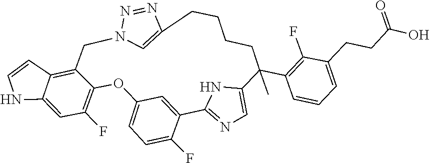 [SMILES: CC1(c2cccc(CCC(=O)O)c2F)CCCCc2cn(nn2)Cc2c(c(F)cc3[nH]ccc23)Oc2ccc(F)c(c2)-c2ncc1[nH]2]

Example 4. Compound 4 23,29-difluoro-6-(2-fluorophenyl)-6-methyl-25-oxa-3,12,13,14,20,32-hexazahexacyclo[24.3.1.12,5.111,14.016,24.017,21]dotriaconta-1(30),2,4,11(31),12,16,18,21,23,26,28-undecaene

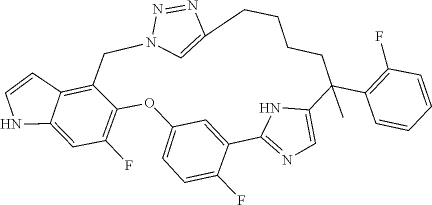 [SMILES: CC1(c2ccccc2F)CCCCc2cn(nn2)Cc2c(c(F)cc3[nH]ccc23)Oc2ccc(F)c(c2)-c2ncc1[nH]2]

Exchanging 1-bromo-3-(3-iodophenyl)-3-methylnon-8-yn-2-one (Intermediate 2-51) with 1-bromo-3-(3-bromo-2-fluorophenyl)-3-methylnon-8-yn-2-one (Intermediate 2-52, 1.22 g, 3.56 mmol), the reaction procedure sequence (Steps A to E) described for Example 2 was used to prepare the title compounds. The Heck coupling product and the debromination product, both originated from corresponding Step C, was not separated until corresponding Step E, after hydrolysis, using reverse phase column. The first eluent is Compound 3(12.7 mg) and the second eluent is Compound 4 (10.8 mg).

Compound 3: MS (ESI): 641 m/z [M+H]⁺. ¹H NMR (400 MHz, CD₃OD) δ 7.48 (s, 1H), 7.35-7.26 (m, 4H), 7.07 (t, J=7.2 Hz, 1H), 6.91-6.77 (m, 4H), 6.31 (d, J=2.8 Hz, 1H), 5.95-5.78 (m, 2H), 2.88 (t, J=7.2 Hz, 2H), 2.72-2.49 (m, 4H), 2.25-1.91 (m, 2H), 1.61-1.51 (m, 4H), 1.38-1.14 (m, 2H), 0.90-0.80 (m, 1H) ppm.

Compound 4: MS (ESI): 569 m/z [M+H]. ¹H NMR (400 MHz, CD₃OD) δ 7.58 (s, 1H), 7.40-7.35 (m, 4H), 7.21-6.85 (m, 6H), 6.30-6.25 (m, 1H), 5.99-5.83 (m, 2H), 2.72-2.57 (m, 1H), 2.30-1.86 (m, 1H), 1.73-1.60 (m, 4H), 1.48-1.21 (m, 4H), 1.01-0.98 (m, 1H) ppm.

Example 5. Compound 5. 3-[3-(24,30-Difluoro-6-methyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

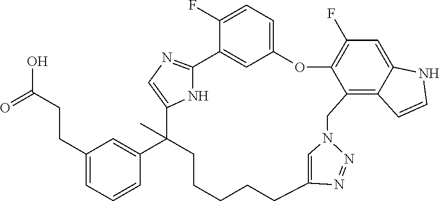 [SMILES: CC1(c2cccc(CCC(=O)O)c2)CCCCCc2cn(nn2)Cc2c(c(F)cc3[nH]ccc23)Oc2ccc(F)c(c2)-c2ncc1[nH]2]

Exchanging 1-bromo-3-(3-iodophenyl)-3-methylnon-8-yn-2-one (Intermediate 2-51) with 1-bromo-3-(3-iodophenyl)-3-methyldec-9-yn-2-one (Intermediate 2-1, 468 mg, 1.17 mmol), the reaction procedure sequence (Steps A to E) described for Example 2 was used to prepare the title compound (60 mg, 60%) as a white solid. MS (ESI): 637 m/z [M+H]⁺, 100% (Purity) (LC-MS Method 2). ¹H NMR (400 MHz, CD₃OD) δ 7.78 (s, 1H), 7.32-7.29 (m, 2H), 7.26 (s, 1H), 7.19-7.16 (m, 2H), 7.14-6.96 (m, 6H), 6.50-6.47 (m, 1H), 5.83-5.77 (m, 2H), 2.83 (t, J=7.8 Hz, 2H), 2.52 (t, J=7.8 Hz, 2H), 2.41-2.22 (m, 2H), 2.11 (t, 1H), 2.02-1.93 (m, 1H), 1.50 (s, 3H), 1.47-1.24 (m, 4H), 1.15-1.08 (m, 1H), 0.93-0.82 (m, 1H) ppm.

Example 6. Compound 6. 3-[3-(24,30-Difluoro-6-methyl-26-oxa-3,11,12,13,21,33-hexazahexacyclo[25.3.1.12,5.110,13.017,25.018,22]tritriaconta-1(31),2,4,10(32),11,17,19,22,24,27,29-undecaen-6-yl)phenyl]propanoic acid

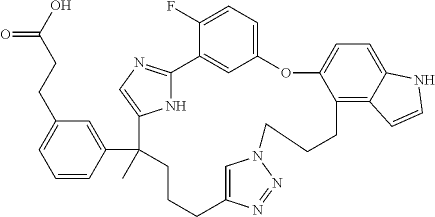 [SMILES: CC1(c2cccc(CCC(=O)O)c2)CCCc2cn(nn2)CCCc2c(ccc3[nH]ccc23)Oc2ccc(F)c(c2)-c2ncc1[nH]2]

Exchanging 5-((4-(azidomethyl)-6-fluoro-1H-indol-5-yl)oxy)-2-fluorobenzimidamide (Intermediate 6-1) with 5-((4-(3-azidopropyl)-6-fluoro-1H-indol-5-yl)oxy)-2-fluorobenzimidamide (Intermediate 6-2, 0.663 g, 1.79 mmol) and 1-bromo-3-(3-iodophenyl)-3-methylnon-8-yn-2-one (Intermediate 2-51) with 1-bromo-3-(3-iodophenyl)-3-methyloct-7-yn-2-one (Intermediate 2-2, 0.75 g, 1.79 mmol), the reaction procedure sequence (Steps A to E) described for Example 2 was used to prepare the title compound (70 mg) as a white solid. MS (ESI): 637 m/z [M+H]⁺. ¹H NMR (400 MHz, CD₃OD) δ 7.63 (s, 1H), 7.61 (s, 1H), 7.46 (s, 1H), 7.33-7.31 (m, 2H), 7.25-7.16 (m, 3H), 7.10-7.06 (m, 3H), 6.62-6.59 (m, 1H), 4.45-4.37 (m, 2H), 3.11-3.00 (m, 2H), 2.92-2.87 (m, 3H), 3.79-2.71 (m, 1H), 2.61-2.57 (m, 2H), 2.42-2.22 (m, 5H), 1.57-1.50 (m, 1H), 1.77 (s, 3H) ppm.

Example 7. Compound 7. 3-[3-(23,29-Difluoro-9,25-dioxa-3,12,13,14,20,32-hexazahexacyclo[24.3.1.12,5.111,14.016,24.017,21]dotriaconta-1(30),2,4,11(31),12,16,18,21,23,26,28-undecaen-6-yl)-2-fluoro-phenyl]propanoic acid

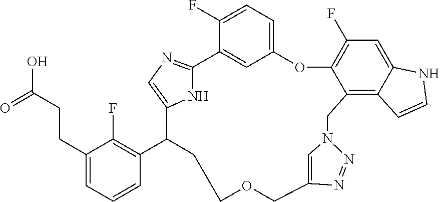 [SMILES: O=C(O)CCc1cccc(C2CCOCc3cn(nn3)Cc3c(c(F)cc4[nH]ccc34)Oc3ccc(F)c(c3)-c3ncc2[nH]3)c1F]

Exchanging 1-bromo-3-(3-iodophenyl)-3-methylnon-8-yn-2-one (Intermediate 2-51) with 1-bromo-3-(3-bromo-2-fluorophenyl)-5-(prop-2-yn-1-yloxy)pentan-2-one (Intermediate 2-3, 1.6 g, 4.1 mmol), the reaction procedure sequence (Steps A to E) described for Example 2 was used to prepare the title compound (400 mg) as a white solid. MS (ESI): 629 m/z [M+H]⁺, retention time: 1.60 minutes, purity: 95% (254 nm) (LC-MS method 6). ¹H NMR (400 MHz, CD₃OD) δ 7.72 (s, 1H), 7.53-7.48 (m, 2H), 7.45-7.36 (m, 2H), 7.29-7.25 (m, 1H), 7.21-7.17 (m, 1H), 7.10-7.05 (m, 2H), 6.83-6.79 (m, 1H), 6.28 (s, 1H), 5.99-5.91 (m, 2H), 4.47-4.41 (m, 1H), 4.36-4.29 (m, 2H), 3.72-3.58 (m, 2H), 2.94-2.90 (m, 2H), 2.58-2.54 (m, 2H), 2.49-2.37 (m, 1H), 2.20-2.10 (m, 1H) ppm.

Example 8. Compounds 8A and 8B. Enantiomers 1 and 2 of 3-[3-(24,30-difluoro-9,26-dioxa-3,13,14,15,21,33-hexazahexacyclo-[25.3.1.12,5.112,15.017,25.018,22]-tritriaconta-1(31),2,4,12(32),13,17,19,22,24,27,29-undecaen-6-yl)-2-fluoro-phenyl]propanoic acid

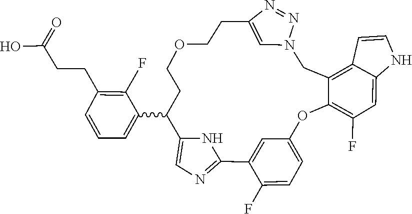 [SMILES: O=C(O)CCc1cccc(C2CCOCCc3cn(nn3)Cc3c(c(F)cc4[nH]ccc34)Oc3ccc(F)c(c3)-c3ncc2[nH]3)c1F]

Exchanging 1-bromo-3-(3-iodophenyl)-3-methylnon-8-yn-2-one (Intermediate 2-51) with 1-bromo-3-(3-bromo-2-fluorophenyl)-5-(but-3-yn-1-yloxy)pentan-2-one (Intermediate 2-4, 0.556 g, 0.00163 mol), the reaction procedure sequence (Steps A to E) described for Example 2 was used to prepare the racemic title compound (140 mg) as a white solid. This racemic acid was subject to chiral SFC separation using SFC-80 (Thar, Waters) under the following condition: Column: OZ 20*250 mm, 10 μm; Column temperature: 35° C.; Mobile phase: carbon dioxide/ethanol (1% 7 N ammonia in methanol as additive)=50/50; Flow rate: 80 g/minute; Back pressure: 100 bar; Detection wavelength: 214 nm; Cycle time: 4 minutes; Sample solution: 140 mg dissolved in 30 mL methanol; Injection volume: 1.8 mL. The first eluent, enantiomer 1, was designated as Compound 8A (59 mg, 100% ee); The second eluent, enantiomer 2, was designated as Compound 8B (54 mg, 100% ee).

Compound 8A: MS (ESI): 643 m/z [M+H]⁺, retention time: 1.63 minutes, purity: >99% (214 nm) (LC-MS method 6). ¹H NMR (400 MHz, CD₃OD) δ 7.47 (s, 1H), 7.39-7.37 (m, 1H), 7.37-7.35 (d, 1H), 7.28-7.26 (m, 2H), 7.16-7.08 (m, 3H), 6.99-6.95 (m, 2H), 6.39-6.37 (m, 1H), 5.93-5.88 (m, 2H), 4.40-4.37 (m, 1H), 3.54-3.44 (m, 4H), 2.94-2.90 (m, 2H), 2.73-2.68 (m, 2H), 2.58-2.54 (m, 2H), 2.34-2.25 (m, 1H), 2.06-1.98 (m, 1H) ppm.

Compound 8B: MS (ESI): 643 m/z [M+H]⁺, retention time: 1.63 minutes, purity: >99% (214 nm) (LC-MS method 6). ¹H NMR (400 MHz, CD₃OD) δ 7.47 (s, 1H), 7.39-7.37 (m, 1H), 7.37-7.35 (m, 1H), 7.28-7.26 (m, 2H), 7.16-7.08 (m, 3H), 6.99-6.94 (m, 2H), 6.39-6.36 (m, 1H), 5.93-5.88 (m, 2H), 4.40-4.36 (m, 1H), 3.55-3.44 (m, 4H), 2.94-2.90 (m, 2H), 2.73-2.68 (m, 2H), 2.58-2.54 (m, 2H), 2.34-2.25 (m, 1H), 2.06-1.98 (m, 1H) ppm.

Example 9. Compound 9. 3-[3-(24,30-Difluoro-26-oxa-3,13,14,15,21,33-hexazahexacyclo-tritriaconta-1(31),2,4,12(32),13,17,19,22,24,27,29-undecaen-6-yl)-2-fluoro-phenyl]propanoic acid

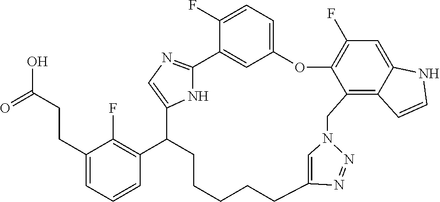 [SMILES: O=C(O)CCc1cccc(C2CCCCCc3cn(nn3)Cc3c(c(F)cc4[nH]ccc34)Oc3ccc(F)c(c3)-c3ncc2[nH]3)c1F]

Exchanging 1-bromo-3-(3-iodophenyl)-3-methylnon-8-yn-2-one (Intermediate 2-51) with 1-bromo-3-(3-bromo-2-fluorophenyl)dec-9-yn-2-one (Intermediate 2-5, 900 mg, 2.24 mmol), the reaction procedure sequence (Steps A to E) described for Example 2 was used to prepare the racemic title compound (80 mg) as a white solid. MS (ESI): 641 m/z [M+H]⁺, retention time: 1.36 minutes, purity: 97% (214 nm) (LC-MS method 7). ¹H NMR (400 MHz, CD₃OD) δ 7.40-7.35 (m, 3H), 7.25-7.23 (m, 2H), 6.91-6.77 (m, 5H), 6.51 (d, J=3.2 Hz, 1H), 5.95-5.78 (m, 2H), 4.24-4.20 (m, 1H), 2.91 (t, J=8.0 Hz, 2H), 2.55 (t, J=8.0 Hz, 2H), 2.40-2.33 (m, 2H), 2.13-1.99 (m, 1H), 1.79-1.67 (m, 1H), 1.51 (t, J=7.2 Hz, 2H), 1.35-1.12 (m, 4H) ppm.

Example 10. Compound 10. 3-[3-(11,11,24,30-Tetrafluoro-6-methyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

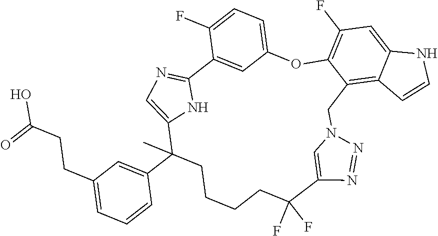 [SMILES: CC1(c2cccc(CCC(=O)O)c2)CCCCC(F)(F)c2cn(nn2)Cc2c(c(F)cc3[nH]ccc23)Oc2ccc(F)c(c2)-c2ncc1[nH]2]

Exchanging 1-bromo-3-(3-iodophenyl)-3-methylnon-8-yn-2-one (Intermediate 2-51) with 1-bromo-8,8-difluoro-3-(3-iodophenyl)-3-methyldec-9-yn-2-one (Intermediate 2-6, 1.24 g, 2.46 mmol), the reaction procedure sequence (Steps A to E) described for Example 2 was used to prepare the racemic title compound (64 mg) as a white solid. MS (ESI): 673 m/z [M+H]⁺, retention time: 1.42 minutes, purity: >99% (254 nm) (LC-MS method 5). 1H NMR (400 MHz, CD₃OD) δ 7.83 (s, 1H), 7.44-7.22 (m, 4H), 7.16-6.97 (m, 6H), 6.69 (d, J=2.7 Hz, 1H), 5.94-5.74 (m, 2H), 2.83 (t, J=7.8 Hz, 2H), 2.51 (t, J=7.8 Hz, 2H), 2.04 (d, J=7.7 Hz, 3H), 1.85 (d, J=4.9 Hz, 1H), 1.50-1.25 (m, 5H), 0.96 (s, 2H) ppm.

Example 11. Compound 11. 3-[3-(24,30-Difluoro-6-methyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)-2-fluoro-phenyl]propanoic acid

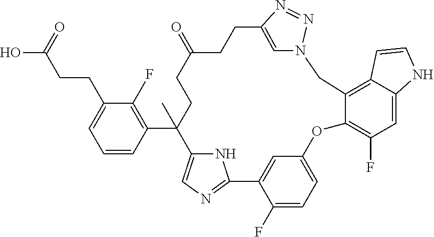 [SMILES: CC1(c2cccc(CCC(=O)O)c2F)CCC(=O)CCc2cn(nn2)Cc2c(c(F)cc3[nH]ccc23)Oc2ccc(F)c(c2)-c2ncc1[nH]2]

Exchanging 1-bromo-3-(3-iodophenyl)-3-methylnon-8-yn-2-one (Intermediate 2-51) with 1-bromo-3-(3-bromo-2-fluorophenyl)-3-methyldec-9-yn-2-one (Intermediate 2-53, 1.24 g, 2.46 mmol), the reaction procedure sequence (Steps A to E) described for Example 2 was used to prepare the racemic title compound (12 mg) as a white solid. MS (ESI): 655 m/z [M+H]⁺, ¹H NMR (400 MHz, CD₃OD) δ 7.40-7.28 (m, 3H), 7.25-7.17 (m, 2H), 7.13-6.99 (m, 3H), 6.95-6.87 (m, 2H), 6.55 (d, J=3.2 Hz, 1H), 5.88 (d, J=14.4 Hz, 1H), 5.77 (d, J=14.4 Hz, 1H), 2.87 (t, J==7.6 Hz, 2H), 2.50 (t, J=7.6 Hz, 2H), 2.36-2.00 (m, 4H), 1.60 (s, 3H), 1.50-1.20 (m, 4H), 1.15-1.02 (m, 1H), 0.95-0.82 (m, 1H) ppm.

Example 12. Compound 12 3-[3-(24,30-Difluoro-11-oxo-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)-2-fluoro-phenyl]propanoic acid

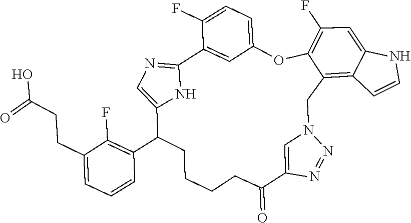 [SMILES: O=C(O)CCc1cccc(C2CCCCC(=O)c3cn(nn3)Cc3c(c(F)cc4[nH]ccc34)Oc3ccc(F)c(c3)-c3ncc2[nH]3)c1F]

and

Example 13. Compound 13. 24,30-Difluoro-6-(2-fluorophenyl)-26-oxa-3,13,14,15,21,33-hexazahexacyclo[25.3.1.12,5.112,15.017,25.018,22]tritriaconta-1(31),2,4,12(32),13,17,19,22,24,27,29-undecaen-11-one

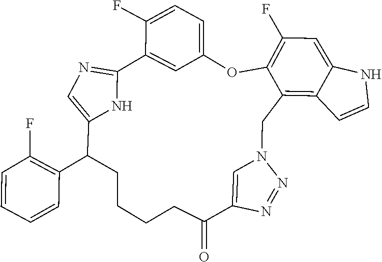 [SMILES: O=C1CCCCC(c2ccccc2F)c2cnc([nH]2)-c2cc(ccc2F)Oc2c(F)cc3[nH]ccc3c2Cn2cc1nn2]

[6-(3-Bromo-2-fluoro-phenyl)-24,30-difluoro-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-11-yl] acetate

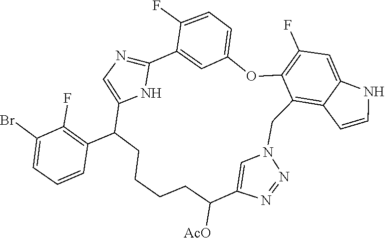 [SMILES: CC(=O)OC1CCCCC(c2cccc(Br)c2F)c2cnc([nH]2)-c2cc(ccc2F)Oc2c(F)cc3[nH]ccc3c2Cn2cc1nn2]

Step A: Exchanging 1-bromo-3-(3-iodophenyl)-3-methylnon-8-yn-2-one (Intermediate 2-51) with 10-bromo-8-(3-bromo-2-fluorophenyl)-9-oxodec-1-yn-3-yl acetate (Intermediate 2-7, 320 mg, 0.69 mmol), the reaction procedure sequence (Steps A to B) described for Example 2 was used to prepare the title compound (258 mg, 53% for two steps) as a yellow solid. MS (ESI): 705, 707 m/z [M+H]⁺, retention time: 1.83 minutes, purity: 80% (254 nm) (LC-MS method 5).

6-(3-Bromo-2-fluoro-phenyl)-24,30-difluoro-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-11-ol

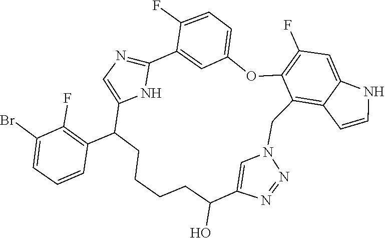 [SMILES: OC1CCCCC(c2cccc(Br)c2F)c2cnc([nH]2)-c2cc(ccc2F)Oc2c(F)cc3[nH]ccc3c2Cn2cc1nn2]

Step B: To a stirred solution of Step A product (258 mg, 0.36 mmol) in tetrahydrofuran (15 mL) was added lithium hydroxide (73 mg, 1.82 mmol). The reaction was stirred at room temperature for 2 hours, cooled to 0° C. and then acidified with 1 M hydrochloric acid to pH ˜4. The mixture was extracted with ethyl acetate (2×30 mL). The combined organic extracts were washed with brine, dried over sodium sulfate, and concentrated. The residue was purified by automated flash chromatography (10 g silica gel column, eluting with 0-50% ethyl acetate in petroleum ether) to give the title compound (65 mg, 27%) as a white oil. MS (ESI): 663, 665 m/z [M+H]⁺, retention time: 1.73 minutes, purity: 90% (254 nm) (LC-MS method 5).

Ethyl 3-[3-(24,30-difluoro-11-hydroxy-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-6-yl)-2-fluoro-phenyl]propanoate

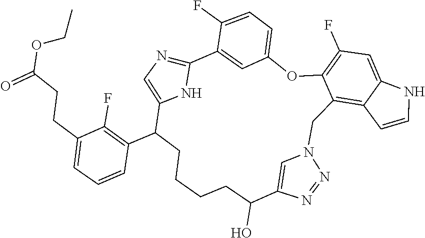 [SMILES: CCOC(=O)CCc1cccc(C2CCCCC(O)c3cn(nn3)Cc3c(c(F)cc4[nH]ccc34)Oc3ccc(F)c(c3)-c3ncc2[nH]3)c1F]

24,30-Difluoro-6-(2-fluorophenyl)-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-11-ol

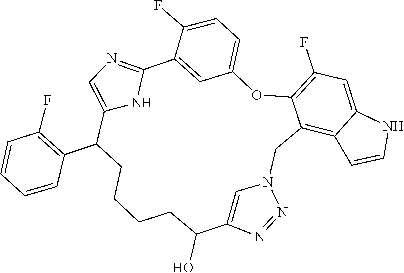 [SMILES: OC1CCCCC(c2ccccc2F)c2cnc([nH]2)-c2cc(ccc2F)Oc2c(F)cc3[nH]ccc3c2Cn2cc1nn2]

Step C: Exchanging 23,29-difluoro-6-(3-iodophenyl)-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaene (Step B product of Example 2) with 6-(3-Bromo-2-fluoro-phenyl)-24,30-difluoro-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-11-ol (Step B product of this example, 400 mg, 0.6 mmol) the reaction procedure sequence (Steps C to D) described for Example 2 was used to prepare the title compounds (320 mg, 78% for two steps) as an inseparable mixture. MS (ESI): 685 m/z [M+H]⁺, retention time: 1.72 minutes, purity: 93% (254 nm) (LC-MS method 5) (for Heck reaction product).

Ethyl 3-[3-(24,30-difluoro-11-oxo-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-6-yl)-2-fluoro-phenyl]propanoate

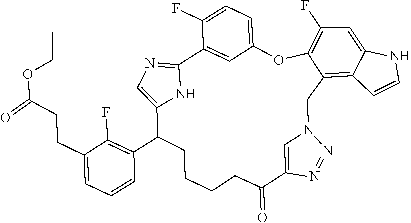 [SMILES: CCOC(=O)CCc1cccc(C2CCCCC(=O)c3cn(nn3)Cc3c(c(F)cc4[nH]ccc34)Oc3ccc(F)c(c3)-c3ncc2[nH]3)c1F]

24,30-difluoro-6-(2-fluorophenyl)-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-11-one

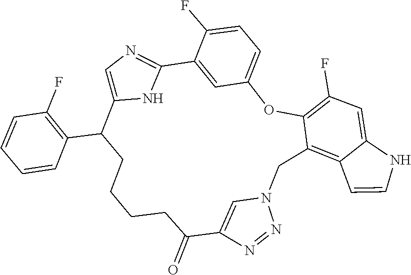 [SMILES: O=C1CCCCC(c2ccccc2F)c2cnc([nH]2)-c2cc(ccc2F)Oc2c(F)cc3[nH]ccc3c2Cn2cc1nn2]

Step D: To a stirred solution of the inseparable mixture of Step C product (220 mg, 0.32 mmol) in dimethyl sulfoxide (15 mL) was added 2-iodoxybenzoic acid (270 mg, 0.96 mmol). The reaction was stirred at room temperature for two hours, then diluted with ethyl acetate (50 mL). The mixture was washed with brine, dried over sodium sulfate, and concentrated. The residue was purified by automated flash chromatography (12 g silica gel column, eluting with 5% methanol in dichloromethane) to give the title mixture (150 mg, 68%) as white oil. MS (ESI): 683 m/z [M+H]⁺, retention time: 1.78 minutes, purity: 93% (254 nm) (LC-MS method 5) (for Heck reaction product).

Compound 12: 3-[3-(24,30-Difluoro-11-oxo-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)-2-fluoro-phenyl]propanoic acid

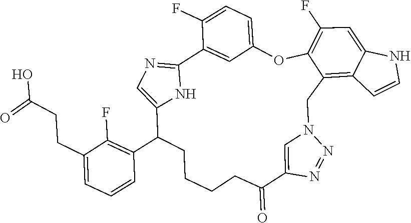 [SMILES: O=C(O)CCc1cccc(C2CCCCC(=O)c3cn(nn3)Cc3c(c(F)cc4[nH]ccc34)Oc3ccc(F)c(c3)-c3ncc2[nH]3)c1F]

Compound 13: 24,30-Difluoro-6-(2-fluorophenyl)-26-oxa-3,13,14,15,21,33-hexazahexacyclo[25.3.1.12,5.112,15.017,25.018,22]tritriaconta-1(31),2,4,12(32),13,17,19,22,24,27,29-undecaen-11-one

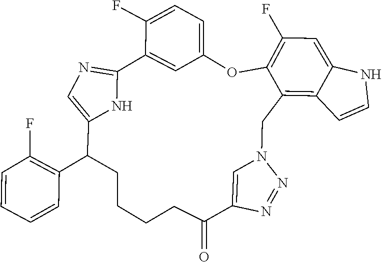 [SMILES: O=C1CCCCC(c2ccccc2F)c2cnc([nH]2)-c2cc(ccc2F)Oc2c(F)cc3[nH]ccc3c2Cn2cc1nn2]

Step E: To a stirred solution of inseparable mixture of Step D product (150 mg, 0.22 mmol) in tetrahydrofuran (15 mL) was added lithium hydroxide (44 mg, 1.10 mmol). The reaction was stirred at room temperature for two hours, cooled to 0° C. and acidified with 1 M hydrochloric acid to pH ˜4. The mixture was extracted with ethyl acetate (2×30 mL). The combined organic extracts were washed with brine, dried over sodium sulfate, and concentrated. The residue was purified by Prep-HPLC. The first eluent is Compound 12(21.4 mg, 15%, white solid). The second eluent is Compound 13 (35.0 mg, 24%, white solid).

Compound 12: MS (ESI): 655 m/z [M+H]⁺, retention time: 1.32 minutes, purity: >99% (254 nm) (LC-MS method 5). ¹H NMR (400 MHz, CD₃OD) δ 8.25 (s, 1H), 7.49 (d, J=3.2 Hz, 1H), 7.40 (d, J=10.7 Hz, 1H), 7.31-7.14 (m, 3H), 7.09 (t, J=7.4 Hz, 1H), 6.96 (t, J=7.7 Hz, 1H), 6.92-6.76 (m, 3H), 6.10-5.90 (m, 2H), 4.15 (dd, J=11.2, 3.5 Hz, 1H), 2.91 (t, J=7.6 Hz, 2H), 2.78-2.67 (m, 1H), 2.55 (t, J=7.7 Hz, 2H), 2.09-1.98 (m, 1H), 1.79-1.76 (m, 2H), 1.56-1.35 (m, 2H), 1.22-1.17 (m, 2H) ppm.

Compound 13: MS (ESI): 583 m/z [M+H]⁺, retention time: 1.71 minutes, purity: >99% (254 nm) (LC-MS method 5). ¹H NMR (400 MHz, DMSO-d₆) δ 11.80 (s, 1H), 11.54 (s, 1H), 8.40 (s, 1H), 7.60-7.57 (m, 1H), 7.44 (d, J=10.9 Hz, 1H), 7.34-7.26 (m, 2H), 7.20-6.92 (m, 5H), 6.76 (d, J=1.8 Hz, 1H), 6.70 (dd, J=5.9, 3.2 Hz, 1H), 6.06-5.92 (m, 2H), 3.99 (dd, J=10.6, 3.9 Hz, 1H), 3.00 (dd, J=10.6, 5.0 Hz, 1H), 2.77-2.64 (m, 1H), 2.03-1.99 (m, 1H), 1.70-1.65 (m, 1H), 1.54-1.40 (m, 2H), 1.14-1.10 (m, 2H) ppm.

Example 14. Compound 14 3-[3-(24,30-Difluoro-11-hydroxy-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)-2-fluoro-phenyl]propanoic acid

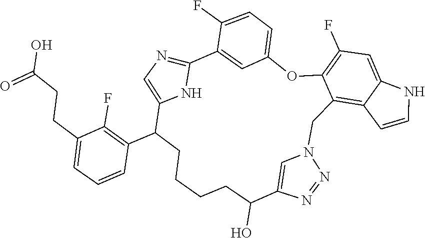 [SMILES: O=C(O)CCc1cccc(C2CCCCC(O)c3cn(nn3)Cc3c(c(F)cc4[nH]ccc34)Oc3ccc(F)c(c3)-c3ncc2[nH]3)c1F]

and

Example 15. Compound 15 24,30-Difluoro-6-(2-fluorophenyl)-26-oxa-3,13,14,15,21,33-hexazahexacyclo[25.3.1.12,5.112,15.017,25.018,22]tritriaconta-1(31),2,4,12(32),13,17,19,22,24,27,29-undecaen-11-ol

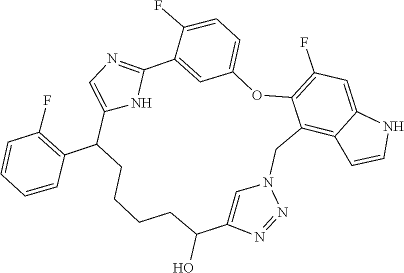 [SMILES: OC1CCCCC(c2ccccc2F)c2cnc([nH]2)-c2cc(ccc2F)Oc2c(F)cc3[nH]ccc3c2Cn2cc1nn2]

To a stirred solution of Step C product of Example 12 (100 mg, 0.14 mmol) in tetrahydrofuran (15 mL) was added lithium hydroxide (29 mg, 0.73 mmol). The reaction was stirred at room temperature for two hours, then acidified with 1 M hydrochloric acid to pH ˜4. The mixture was extracted with ethyl acetate (2×30 mL). The combined organic extracts were washed with brine (2×30 mL), dried over sodium sulfate, and concentrated. The residue was purified by Prep-HPLC to afford the two title compounds. The first eluent is Compound 14 (18.3 mg, 18.0%) and the second eluent is Compound 15 (27.8 mg, 27.0%), both as a white solid.

Compound 14: MS (ESI): 657 m/z [M+H]⁺, retention time: 1.61 minutes, purity: >99% (254 nm) (LC-MS method 5). ¹H NMR (400 MHz, CD₃OD) δ 7.59-7.54 (m, 1H), 7.45-7.36 (m, 2H), 7.30-7.22 (m, 2H), 7.19-7.05 (m, 2H), 7.00-6.90 (m, 3H), 6.72 (d, J=3.0 Hz, 1H), 6.04-5.83 (m, 2H), 4.47 (d, J=7.2 Hz, 1H), 4.30-4.16 (m, 1H), 2.91 (t, J=7.9 Hz, 2H), 2.55 (t, J=7.7 Hz, 2H), 2.10-2.00 (m, 1H), 1.71-1.60 (m, 3H), 1.36-1.28 (m, 4H) ppm.

Compound 15: MS (ESI): 585 m/z [M+H]⁺, retention time: 1.67 minutes, purity: 93% (254 nm) (LC-MS method 5). ¹H NMR (400 MHz, DMSO-d₆) δ 11.92 (d, J=11.4 Hz, 1H), 11.48 (s, 1H), 7.52-6.91 (m, 11H), 6.73 (d, J=10.1 Hz, 1H), 5.93-5.78 (m, 2H), 5.01-4.89 (m, 1H), 4.47 (s, 1H), 4.25 (d, J=7.8 Hz, 1H), 2.04-1.94 (m, 1H), 1.62-1.47 (m, 3H), 1.30-1.18 (m, 4H) ppm.

Example 16. Compound 16. 3-[3-(24,30-Difluoro-16-hydroxy-6-methyl-26-oxa-3,12,13,14,21,33-hexazahexacyclo[25.3.1.12,5.112,15.017,25.018,22]tritriaconta-1(31),2,4,13,15(32),17,19,22,24,27,29-undecaen-6-yl)phenyl]propanoic acid

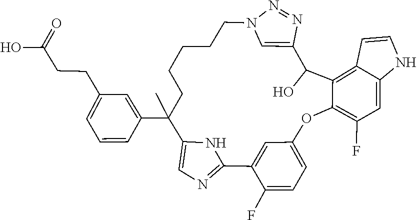 [SMILES: CC1(c2cccc(CCC(=O)O)c2)CCCCCn2cc(nn2)C(O)c2c(c(F)cc3[nH]ccc23)Oc2ccc(F)c(c2)-c2ncc1[nH]2]

Ethyl 3-[3-(24,30-difluoro-16-hydroxy-6-methyl-26-oxa-3,12,13,14,21,33-hexazahexacyclo[25.3.1.12,5.112,15.017,25.018,22]tritriaconta-1(31),2,4,13,15(32),17,19,22,24,27,29-undecaen-6-yl)phenyl]propanoate

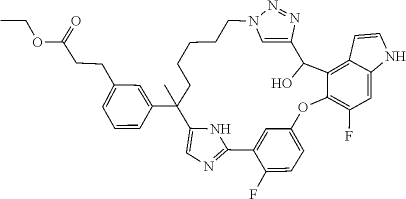 [SMILES: CCOC(=O)CCc1cccc(C2(C)CCCCCn3cc(nn3)C(O)c3c(c(F)cc4[nH]ccc34)Oc3ccc(F)c(c3)-c3ncc2[nH]3)c1]

Step A: Exchanging 5-((4-(azidomethyl)-6-fluoro-1H-indol-5-yl)oxy)-2-fluorobenzimidamide (Intermediate 6-1) with 2-fluoro-5-((6-fluoro-4-(1-hydroxyprop-2-yn-1-yl)-1H-indol-5-yl)oxy)benzimidamide (Intermediate 6-4, 1.42 g, 4.16 mmol) and 1-bromo-3-(3-iodophenyl)-3-methylnon-8-yn-2-one (Intermediate 2-51) with 8-azido-1-bromo-3-(3-iodophenyl)-3-methyloctan-2-one (Intermediate 2-8, 869 mg, 1.87 mmol), the reaction procedure sequence (Steps A to D) described for Example 2 was used to prepare the racemic title compound (12 mg) as a white solid. MS (ESI): 681 m/z [M+H]⁺, retention time: 1.69 minutes, purity: >99% (214 nm) (LC-MS method 2).

Compound 16: 3-[3-(24,30-Difluoro-16-hydroxy-6-methyl-26-oxa-3,12,13,14,21,33-hexazahexacyclo[25.3.1.12,5.112,15.017,25.018,22]tritriaconta-1(31),2,4,13,15(32),17,19,22,24,27,29-undecaen-6-yl)phenyl]propanoic acid

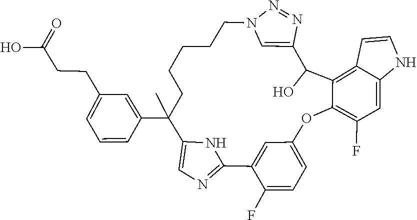 [SMILES: CC1(c2cccc(CCC(=O)O)c2)CCCCCn2cc(nn2)C(O)c2c(c(F)cc3[nH]ccc23)Oc2ccc(F)c(c2)-c2ncc1[nH]2]

Step B: To a stirred solution of Step A product (12 mg, 0.017 mmol) in tetrahydrofuran/water/methanol (3 mL/1 mL/1 mL) was added lithium hydroxide monohydrate (3.6 mg, 0.09 mmol). The reaction was stirred at room temperature for 1.5 hours, then acidified with 1 N hydrochloric acid to pH ˜4 and diluted with ethyl acetate (30 mL). The solution was washed with brine, dried over sodium sulfate, and concentrated to give the title compound (9.4 mg, 82%) as a solid. MS (ESI): 653 m/z [M+H]⁺, retention time: 1.58 minutes, purity: >99% (214 nm) (LC-MS method 2). 1H NMR (400 MHz, CD₃OD) δ 7.72-7.61 (m, 1H), 7.38-6.77 (m, 10H), 6.74-6.65 (m, 1H), 6.53-6.44 (m, 1H), 4.23-4.09 (m, 2H), 2.89-2.82 (m, 2H), 2.59-5.51 (m, 2H), 2.27-1.90 (m, 2H), 1.67-1.28 (m, 7H), 0.96-0.84 (m, 2H) ppm.

Example 17. Compound 17. 3-[3-(24,30-Difluoro-6-methyl-16-oxo-26-oxa-3,12,13,14,21,33-hexazahexacyclo[25.3.1.12,5.112,15.017,25.018,22]tritriaconta-1(31),2,4,13,15(32),17,19,22,24,27,29-undecaen-6-yl)phenyl]propanoic acid

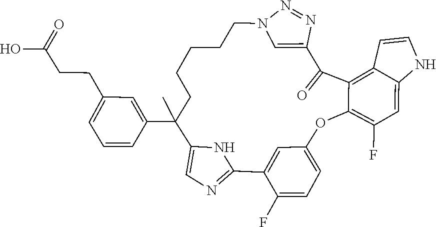 [SMILES: CC1(c2cccc(CCC(=O)O)c2)CCCCCn2cc(nn2)C(=O)c2c(c(F)cc3[nH]ccc23)Oc2ccc(F)c(c2)-c2ncc1[nH]2]

Ethyl 3-[3-(24,30-difluoro-6-methyl-16-oxo-26-oxa-3,12,13,14,21,33-hexazahexacyclo-[25.3.1.12,5.112,15.017,25.018,22]tritriaconta-1(31),2,4,13,15(32),17,19,22,24,27,29-undecaen-6-yl)phenyl]propanoate

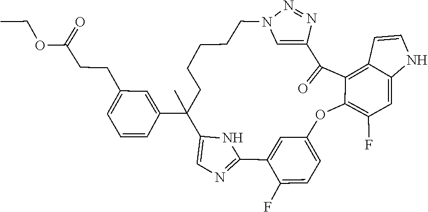 [SMILES: CCOC(=O)CCc1cccc(C2(C)CCCCCn3cc(nn3)C(=O)c3c(c(F)cc4[nH]ccc34)Oc3ccc(F)c(c3)-c3ncc2[nH]3)c1]

Step A: To a stirred solution of Step A product of Example 16 (65 mg, 0.095 mmol) in tetrahydrofuran (6 mL) was added manganese dioxide (83 mg, 0.95 mmol). The mixture was stirred at room temperature overnight, then filtered through a pad of Celite. The filtrate was concentrated. The residue was purified by automated flash chromatography (4 g silica gel column, eluting with 0-30% ethyl acetate in petroleum ether) to give the title compound (45 mg, 69%) as a white solid. MS (ESI): 679 m/z [M+H]⁺, retention time: 1.77 minutes, purity: 59% (214 nm) (LC-MS method 2).

Compound 17: 3-[3-(24,30-Difluoro-6-methyl-16-oxo-26-oxa-3,12,13,14,21,33-hexazahexacyclo[25.3.1.12,5.112,15.017,25.018,22]tritriaconta-1(31),2,4,13,15(32),17,19,22,24,27,29-undecaen-6-yl)phenyl]propanoic acid

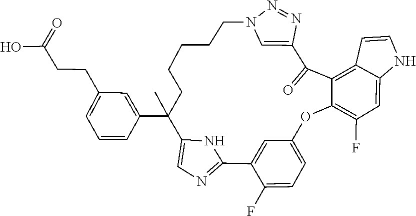 [SMILES: CC1(c2cccc(CCC(=O)O)c2)CCCCCn2cc(nn2)C(=O)c2c(c(F)cc3[nH]ccc23)Oc2ccc(F)c(c2)-c2ncc1[nH]2]

To a solution of Step A product (45 mg, 0.066 mmol) in tetrahydrofuran/water/methanol (3 mL/1 mL/1 mL) was added lithium hydroxide monohydrate (13.6 mg, 0.33 mmol). The reaction was stirred at room temperature for 1.5 hours, then acidified with 1 M hydrochloric acid to pH ˜4. The mixture was diluted with ethyl acetate (50 mL), washed with brine, dried over sodium sulfate, and concentrated. The residue was purified by prep-HPLC to give the title compound (16.7 mg, 38.8%) as a white solid. MS (ESI): 651 m/z [M+H]⁺, retention time: 1.65 minutes, purity: 98% (214 nm) (LC-MS method 2). ¹H NMR (400 MHz, CD₃OD) δ 8.67 (s, 1H), 7.50 (d, J=10.3 Hz, 1H), 7.39 (d, J=3.2 Hz, 1H), 7.33-7.30 (m, 1H), 7.21-6.91 (m, 6H), 6.74-6.71 (m, 1H), 6.42 (d, J=2.5 Hz, 1H), 4.29-4.13 (m, 2H), 2.86 (t, J=7.7 Hz, 2H), 2.53 (t, J=7.8 Hz, 2H), 2.35 (d, J=11.4 Hz, 1H), 2.15-2.11 (m, 1H), 1.94-1.90 (m, 1H), 1.68-1.28 (m, 7H), 0.88 (d, J=9.6 Hz, 1H) ppm.

Example 18. Compound 18. 3-[3-(24,30-Difluoro-6,9-dimethyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

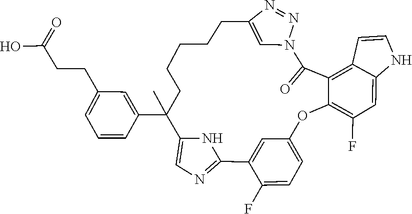 [SMILES: CC1(c2cccc(CCC(=O)O)c2)CCCCCc2cn(nn2)C(=O)c2c(c(F)cc3[nH]ccc23)Oc2ccc(F)c(c2)-c2ncc1[nH]2]

Exchanging 1-bromo-3-(3-iodophenyl)-3-methylnon-8-yn-2-one (Intermediate 2-51) with 1-bromo-3-(3-iodophenyl)-3-methyl-6-(prop-2-yn-1-yloxy)heptan-2-one (Intermediate 2-9, 1.2 g, 2.59 mmol), the reaction procedure sequence (Steps A to E) described for Example 2 was used to prepare the racemic title compound (220 mg) as a white solid. MS (ESI): 653 m/z [M+H]⁺, retention time: 1.67 minutes, purity: >99% (214 nm) (LC-MS method 2). (mixture of 4 diastereomers)

Example 19. Compound 19A and 19B. Diastereomers 1 and 2 of 3-[3-(24,30-difluoro-6,9-dimethyl-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-6-yl)phenyl]propanoic acid

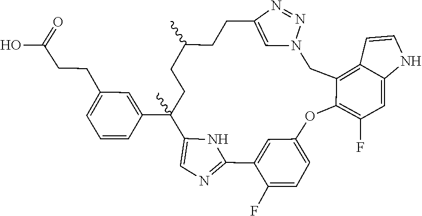

Exchanging 1-bromo-3-(3-iodophenyl)-3-methylnon-8-yn-2-one (Intermediate 2-51) with 1-bromo-3-(3-iodophenyl)-3,6-dimethyldec-9-yn-2-one (Intermediate 2-10, 900 mg (85% purity), 2.36 mmol), the reaction procedure sequence (Steps A to E) described for Example 2 was used to prepare the title compounds. Due to the existence of two chiral center, two potential cis/trans isomers (each has two enantiomers) were separated by silica gel column chromatography, after Heck coupling, corresponding to Step C of Example 2. The first eluent, the first diastereomer (121 mg), was subjected to Steps D and E conditions of Example 2, to give Compound 19A (39.9 mg, white solid). The second eluent, the second diastereomer (161 mg), was subject to same reaction sequence (Steps D and E) to give Compound 19B (35.2 mg) as a white solid.

Compound 19A: MS (ESI): 651 m/z [M+H]⁺, retention time: 1.56 minutes, purity: >99% (254 nm) (LC-MS method 5). ¹H NMR (400 MHz, DMSO-d₆) δ 11.86 (s, 1H), 11.50 (s, 1H), 7.49-7.37 (m, 2H), 7.34-7.24 (m, 1H), 7.24-6.81 (m, 8H), 6.46 (s, 1H), 5.85-5.70 (m, 2H), 2.68 (t, J=7.7 Hz, 2H), 2.40 (t, J=7.8 Hz, 2H), 2.32-2.20 (m, 2H), 2.15-2.03 (m, 1H), 1.60 (d, J=5.8 Hz, 1H), 1.40-1.32 (m, 4H), 1.25-0.94 (m, 4H), 0.75 (t, J=9.5 Hz, 3H) ppm.

Compound 19B: MS (ESI): 651 m/z [M+H]⁺, retention time: 1.56 minutes, purity: 95% (254 nm) (LC-MS method 5). ¹H NMR (400 MHz, DMSO-d₆) δ 11.84 (s, 1H), 11.53 (s, 1H), 7.54-7.42 (m, 2H), 7.35-6.88 (m, 9H), 6.64 (s, 1H), 5.85-5.70 (m, 2H), 2.69 (t, J=7.5 Hz, 2H), 2.42 (t, J=7.6 Hz, 2H), 2.32-2.06 (m, 3H), 1.69-1.66 (m, 1H), 1.40-1.32 (m, 4H), 1.25-0.95 (m, 4H), 0.79 (d, J=6.2 Hz, 3H) ppm.

Example 20. Compound 20A and 20B. Enantiomers 1 and 2 of methyl 3-[3-(24,30-difluoro-6-methyl-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-6-yl)phenyl]propanoate

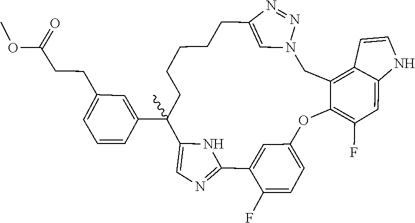 [SMILES: COC(=O)CCc1cccc(C2(C)CCCCCc3cn(nn3)Cc3c(c(F)cc4[nH]ccc34)Oc3ccc(F)c(c3)-c3ncc2[nH]3)c1]

Exchanging 1-bromo-3-(3-iodophenyl)-3-methylnon-8-yn-2-one (Intermediate 2-51) with 1-bromo-3-(3-iodophenyl)-3-methyldec-9-yn-2-one (Intermediate 2-11, 2.25 g, 5.1 mmol), the reaction procedure sequence (Steps A to D) described for Example 2 was used to prepare the title compounds. The racemic methyl ester (1 g), obtained in corresponding Step D of Example 2, was subject to chiral SFC separation under the following condition: Instrument: SFC-80 (Thar, Waters); Column: OJ 20*250 mm, 10 μm; Column temperature: 35° C. Mobile phase: carbon dioxide/ethanol (1% (7 M ammonia in methanol) as additive)=70/30; Flow rate: 80 g/minutes; Back pressure: 100 bar; Detection wavelength: 214 nm; Cycle time: 4.5 minutes; Sample solution: 160 mg dissolved in 15 mL methanol. Injection volume: 0.6 mL. The first eluent, Enantiomer 1 (9.4 mg), was designated as Compound 20A; The second eluent, Enantiomer 2 (6.8 mg), was designated as Compound 20B.

Compound 20A: MS (ESI): 651 m/z [M+H]⁺, retention time: 1.74 minutes, purity: >99% (254 nm) (LC-MS method 2). ¹H-NMR (400 MHz, CD₃OD) δ: 7.37-7.03 (m, 10H), 6.95 (d, J=6.8 Hz, 1H), 6.53 (s, 1H), 5.87 (d, J=14.5 Hz, 1H), 5.75 (d, J=15.1 Hz, 1H), 3.58 (s, 3H), 2.82 (t, J=7.2 Hz, 2H), 2.54 (t, J=7.2 Hz, 2H), 2.20-2.40 (m, 3H), 1.90-1.93 (m, 1H), 1.50-1.38 (m, 4H), 1.31-1.13 (m, 4H), 0.83-0.90 (m, 1H) ppm.

Compound 20B: MS (ESI): 651 m/z [M+H]⁺, retention time: 1.74 minutes, purity: >99% (254 nm) (LC-MS method 2). ¹H-NMR (400 MHz, CD₃OD) δ: 7.37-7.07 (m, 10H), 6.92 (d, J=6.8 Hz, 1H), 6.51 (s, 1H), 5.89 (d, J=14.5 Hz, 1H), 5.75 (d, J=14.5 Hz, 1H), 3.58 (s, 3H), 2.82 (t, J=7.2 Hz, 2H), 2.54 (t, J=7.2 Hz, 2H), 2.20-2.40 (m, 3H), 1.90-1.93 (m, 1H), 1.50-1.38 (m, 4H), 1.31-1.13 (m, 4H), 0.83-0.90 (m, 1H) ppm.

Example 21. Compound 21 3-[3-(23,29-Difluoro-6-methyl-25,31-dioxa-3,12,20,32-tetrazahexacyclo[24.3.1.12,5.110,13.016,24.017,21]dotriaconta-1(30),2,4,10,12,16,18,21,23,26,28-undecaen-6-yl)phenyl]propanoic acid

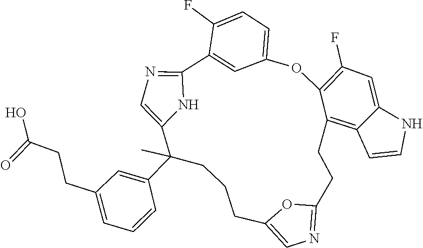 [SMILES: CC1(c2cccc(CCC(=O)O)c2)CCCC2=CN=C(CCc3c(c(F)cc4[nH]ccc34)Oc3ccc(F)c(c3)-c3ncc1[nH]3)O2]

Methyl (E)-3-(5-(3-(5-(6-acetoxy-7-azido-2-(3-iodophenyl)heptan-2-yl)-1H-imidazol-2-yl)-4-fluorophenoxy)-6-fluoro-1H-indol-4-yl)acrylate

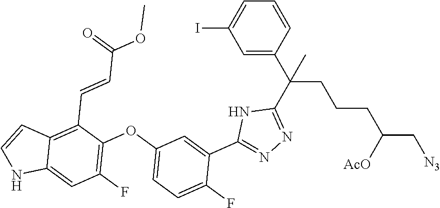 [SMILES: COC(=O)/C=C/c1c(Oc2ccc(F)c(-c3nnc(C(C)(CCCC(CN=[N+]=[N-])OC(C)=O)c4cccc(I)c4)[nH]3)c2)c(F)cc2[nH]ccc12]

Step A: To a stirred solution of 1-azido-8-bromo-6-(3-iodophenyl)-6-methyl-7-oxooctan-2-yl acetate (Intermediate 2-12, 1 g, 1.91 mmol) in dimethylformamide (10 mL) was added methyl (E)-3-(5-(3-carbamimidoyl-4-fluorophenoxy)-6-fluoro-1H-indol-4-yl)acrylate (Intermediate 6-5, 854 mg, 2.30 mmol), sodium bicarbonate (791 mg, 5.73 mmol). The reaction was stirred at 80° C. for 2 hours and concentrated. The residue was partitioned between water (50 mL) and ethyl acetate (100 mL). The separated organic phase, combined with two additional ethyl acetate extracts, was dried over magnesium sulfate, and concentrated. The crude product was purified by prep-TLC eluting with 40% ethyl acetate/petroleum ether to provide the title compound (1 g, 46%) as light red oil. MS (ESI): 795 m/z [M+H]⁺, retention time: 1.92 minutes, purity: 93% (214 nm) (LC-MS method 2).

(E)-3-(5-(3-(5-(7-Azido-6-hydroxy-2-(3-iodophenyl)heptan-2-yl)-1H-imidazol-2-yl)-4-fluorophenoxy)-6-fluoro-1H-indol-4-yl)acrylic acid

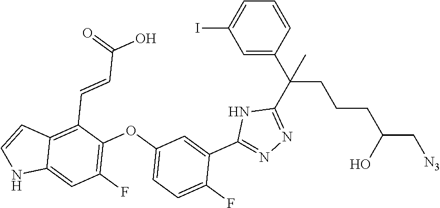 [SMILES: CC(CCCC(O)CN=[N+]=[N-])(c1cccc(I)c1)c1nnc(-c2cc(Oc3c(F)cc4[nH]ccc4c3/C=C/C(=O)O)ccc2F)[nH]1]

Step B: To a stirred solution of Step A product (800 mg, 1 mmol) in 5 mL of methanol, 5 mL of tetrahydrofuran and 5 mL of water was added lithium hydroxide monohydrate (460 mg, 20 mmol). The mixture was stirred at room temperature for 18 hours and concentrated. The residue was acidified with 1 N hydrochloric acid to pH ˜2 and partitioned between water (20 mL) and ethyl acetate (20 mL). The separated organic layer, combined with one additional ethyl acetate extract (1×30 mL), was washed with brine, dried over magnesium sulfate, and concentrated. The crude product was purified by preparative thin layer chromatography to give the title compound (720 mg, 90%) as a white solid. MS (ESI): 739 m/z [M+H]⁺, retention time: 1.32 minutes, purity: 97% (214 nm) (LC-MS method 2).

(E)-3-(5-(3-(5-(7-Amino-6-hydroxy-2-(3-iodophenyl)heptan-2-yl)-1H-imidazol-2-yl)-4-fluorophenoxy)-6-fluoro-1H-indol-4-yl)acrylic acid

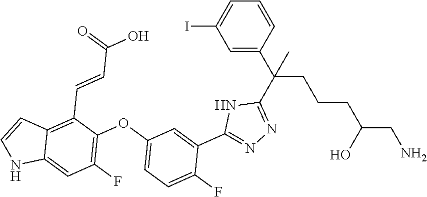 [SMILES: CC(CCCC(O)CN)(c1cccc(I)c1)c1nnc(-c2cc(Oc3c(F)cc4[nH]ccc4c3/C=C/C(=O)O)ccc2F)[nH]1]

Step C: To a stirred solution of Step B product (1 g, 1.35 mmol) in 50 mL of tetrahydrofuran and 5 mL water was added triphenylphosphine (426 mg, 1.62 mmol). The mixture was stirred at 70° C. for 18 hours and concentrated. The residue was triturated with dichloromethane to give the title compound (800 mg, 80%) as a white solid. MS (ESI): 713 m/z [M+H]⁺, retention time: 1.32 minutes, purity: 88% (214 nm) (LC-MS method 2).

(14E)-23,29-Difluoro-10-hydroxy-6-(3-iodophenyl)-6-methyl-25-oxa-3,12,20,31-tetrazapentacyclo[24.3.1.12,5.016,24.017,21]hentriaconta-1(30),2,4,14,16,18,21,23,26,28-decaen-13-one

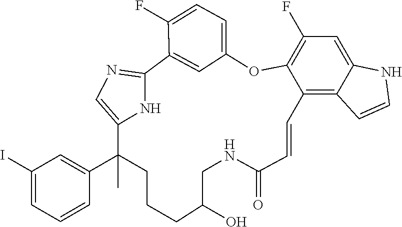 [SMILES: CC1(c2cccc(I)c2)CCCC(O)CNC(=O)/C=C/c2c(c(F)cc3[nH]ccc23)Oc2ccc(F)c(c2)-c2ncc1[nH]2]

Step D: To a solution of Step C product (800 mg, 1.12 mmol) in 10 mL of tetrahydrofuran and 1 mL of dimethylformamide was added 1-ethyl-3-(3-dimethylaminopropyl)carbodiimide hydrochloride (429 mg, 2.24 mmol) and hydroxybenzotriazole (302 mg, 2.24 mmol). The reaction was stirred at room temperature for 18 hours under nitrogen, then partitioned between ethyl acetate (30 mL) and water (30 mL). The separated organic layer, combined with two additional ethyl acetate extracts (2×20 mL), was dried over magnesium sulfate, and concentrated. The residue was purified by automated chromatography (12 g silica gel column, eluted with 90% ethyl acetate/hexanes) to provide the title compound (300 mg, 40%) as a yellow oil. MS (ESI): 695 m/z [M+H]⁺, retention time: 1.58 minutes, purity: 90% (214 nm) (LC-MS method 2).

Methyl (E)-3-[3-[(14E)-23,29-difluoro-10-hydroxy-6-methyl-13-oxo-25-oxa-3,12,20,31-tetrazapentacyclo[24.3.1.12,5.016,24.017,21]hentriaconta-1(30),2,4,14,16,18,21,23,26,28-decaen-6-yl]phenyl]prop-2-enoate

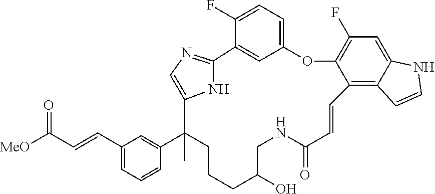 [SMILES: COC(=O)/C=C/c1cccc(C2(C)CCCC(O)CNC(=O)/C=C/c3c(c(F)cc4[nH]ccc34)Oc3ccc(F)c(c3)-c3ncc2[nH]3)c1]

Step E: To a stirred and degassed solution of Step D product (60 mg, 0.09 mmol) in 5 mL of dimethylformamide were added methyl acrylate (810 mg, 9.57 mmol), tri-o-tolyl phosphine (290 mg, 0.95 mmol), triethylamine (966 mg, 9.57 mmol) and palladium (II) acetate (140 mg, 0.2 mmol). The mixture was stirred at 100° C. for 18 hours under nitrogen, cooled to room temperature, partitioned between water (30 mL) and ethyl acetate (50 mL). The separated organic layer, combined with two additional ethyl acetate extracts (2×20 mL), was washed with 1 N hydrochloric acid, brine, dried over magnesium sulfate, and concentrated. The residue was purified by automated flash chromatography (12 g silica gel column, 0-90% ethyl acetate/hexanes) to provide the title compound (30 mg, 50%) as yellow oil. MS (ESI): 653 m/z [M+H]⁺, retention time: 1.20 minutes, purity: 90% (214 nm) (LC-MS method 2).

Methyl 3-[3-(23,29-difluoro-10-hydroxy-6-methyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]-propanoate

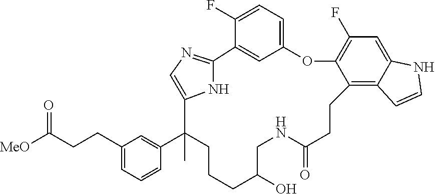 [SMILES: COC(=O)CCc1cccc(C2(C)CCCC(O)CNC(=O)CCc3c(c(F)cc4[nH]ccc34)Oc3ccc(F)c(c3)-c3ncc2[nH]3)c1]

Step F: To a stirred solution of Step E product (0.7 g, 0.11 mmol) in 100 mL of methanol was added palladium on carbon (10%, ˜50% wet, 0.7 g). The mixture was stirred for 2 hours at room temperature under hydrogen (balloon), then filtered through a pad of Celite. The filtrate was concentrated to give the title compound (60 mg, 85%) as a yellow oil. MS (ESI): 657 m/z [M+H]⁺, retention time: 1.96 minutes, purity: 95% (214 nm) (LC-MS method 2).

Methyl 3-[3-(23,29-difluoro-6-methyl-10,13-dioxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoate

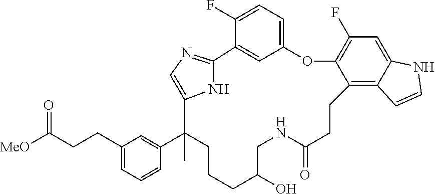 [SMILES: COC(=O)CCc1cccc(C2(C)CCCC(O)CNC(=O)CCc3c(c(F)cc4[nH]ccc34)Oc3ccc(F)c(c3)-c3ncc2[nH]3)c1]

Step G: To a stirred solution of Step F product (60 mg, 0.09 mmol) in 5 mL of dimethyl sulfoxide was added 2-iodoxybenzoic acid (77 mg, 9.57 mmol). The mixture was stirred at 45° C. for 18 hours. The mixture was diluted with water (10 mL) and extracted with ethyl acetate (3×10 mL). The combined organic phases were washed with brine, dried over magnesium sulfate, and concentrated. The residue was purified by automated flash chromatography (4 g silica gel column, 0-100% ethyl acetate/hexanes) to provide the title compound (30 mg, 50%) as yellow oil. MS (ESI): 655 m/z [M+H]⁺, retention time: 1.20 minutes, purity: 90% (214 nm) (LC-MS method 2).

Methyl 3-[3-(23,29-Difluoro-6-methyl-25,31-dioxa-3,12,20,32-tetrazahexacyclo-[24.3.1.12,5.110,13.016,24.017,21]dotriaconta-1(30),2,4,10,12,16,18,21,23,26,28-undecaen-6-yl)phenyl]propanoate

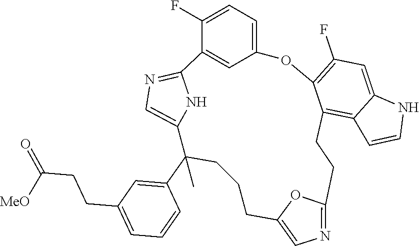 [SMILES: COC(=O)CCc1cccc(C2(C)CCCC3=CN=C(CCc4c(c(F)cc5[nH]ccc45)Oc4ccc(F)c(c4)-c4ncc2[nH]4)O3)c1]

Step H: To a stirred solution of Step G product (60 mg, 0.09 mmol) in 5 mL of toluene was added propane phosphonic acid anhydride (286 mg, 0.9 mmol). The reaction was stirred at 120° C. for 4 hours, cooled to room temperature and partitioned between water (15 mL) and ethyl acetate (20 mL). The separated organic layer, combined with two additional ethyl acetate (2×15 mL) extracts, was dried over magnesium sulfate, and concentrated. The residue was purified by automated flash chromatography (4 g silica gel column, 0-100% ethyl acetate/hexanes) to provide the title compound (30 mg, 50%) as a yellow oil. MS (ESI): 637 m/z [M+H]⁺, retention time: 1.85 minutes, purity: 90% (214 nm) (LC-MS method 2).

Compound 21: 3-[3-(23,29-Difluoro-6-methyl-25,31-dioxa-3,12,20,32-tetrazahexacyclo[24.3.1.12,5.110,13.016,24.017,21]dotriaconta-1(30),2,4,10,12,16,18,21,23,26,28-undecaen-6-yl)phenyl]propanoic acid

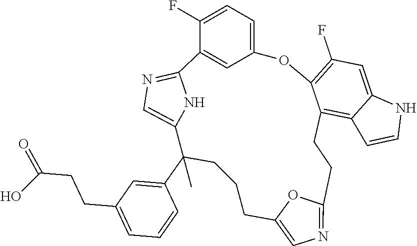 [SMILES: CC1(c2cccc(CCC(=O)O)c2)CCCC2=CN=C(CCc3c(c(F)cc4[nH]ccc34)Oc3ccc(F)c(c3)-c3ncc1[nH]3)O2]

Step I. To a stirred solution of Step H product (20 mg, 0.03 mmol) in 5 mL of methanol and 2 mL water was added lithium hydroxide monohydrate (12 mg, 0.6 mmol). The mixture was stirred at room temperature for 2 hours and concentrated. The residue was diluted with water, acidified with 2 N hydrochloric acid to pH ˜3. The mixture was extracted with ethyl acetate for (3×10 mL). The combined organic layers were dried over sodium sulfate and concentrated. The obtained solid was triturated with ethyl acetate to afford the title compound (16 mg, 80%) as a white solid. MS (ESI): 623 m/z [M+H]⁺, retention time: 1.60 minutes, purity: 95% (214 nm) (LC-MS method 6). ¹H NMR (400 MHz, CD₃OD) δ 7.44-7.50 (m, 3H), 7.19-7.25 (m, 4H), 7.15-7.17 (m, 2H), 7.10-7.12 (m, 1H), 6.63 (s, 1H), 6.19-6.16 (m, 1H), 3.32-3.33 (m, 2H), 2.88-2.92 (m, 2H), 2.84-2.86 (m, 2H), 2.52-2.56 (m, 2H), 2.35-2.37 (m, 2H), 2.09 (m, 1H), 1.92 (m, 1H), 1.62 (s, 3H), 1.28-1.39 (m, 2H) ppm.

Example 22. Compound 22. 3-[3-(23,29-Difluoro-6-methyl-25-oxa-3,12,20,31,32-pentazahexacyclo[24.3.1.12,5.110,13.016,24.017,21]dotriaconta-1(30),2,4,10,12,16,18,21,23,26,28-undecaen-6-yl)phenyl]propanoic acid

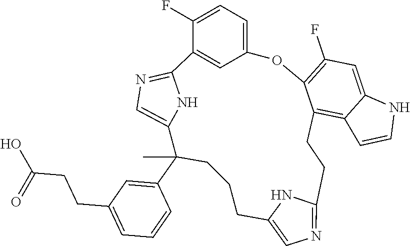 [SMILES: CC1(c2cccc(CCC(=O)O)c2)CCCc2cnc([nH]2)CCc2c(c(F)cc3[nH]ccc23)Oc2ccc(F)c(c2)-c2ncc1[nH]2]

Methyl 3-[3-(23,29-difluoro-6-methyl-25-oxa-3,12,20,31,32-pentazahexacyclo-[24.3.1.12,5.110,13.016,24.017,21]dotriaconta-1(30),2,4,10,12,16,18,21,23,26,28-undecaen-6-yl)phenyl]propanoate

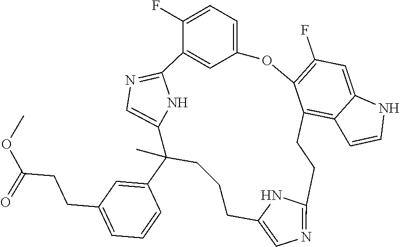 [SMILES: COC(=O)CCc1cccc(C2(C)CCCc3cnc([nH]3)CCc3c(c(F)cc4[nH]ccc34)Oc3ccc(F)c(c3)-c3ncc2[nH]3)c1]

Step A: To a stirred solution of methyl 3-[3-(23,29-difluoro-6-methyl-10,13-dioxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoate (Step G product of Example 21, 60 mg, 0.09 mmol) in 5 mL of N-methyl morpholine and acetic acid (1 ml) was added ammonium acetate (110 mg, 0.9 mmol). The reaction was stirred at 140° C. for 4 hours in a microwave reactor. The mixture was partitioned between water (20 mL) and ethyl acetate (20 mL). The separated organic layer, combined with additional two ethyl acetate extracts (2×15 mL), was dried over magnesium sulfate, and concentrated. The residue was purified by automated flash chromatography (4 g silica gel column, eluting with 0-10% methanol in dichloromethane) to provide the title compound (30 mg, 50%) as yellow oil. MS (ESI): 636 m/z [M+H]⁺, retention time: 1.85 minutes, purity: 92% (214 nm) (LC-MS method 2).

Compound 22: 3-[3-(23,29-difluoro-6-methyl-25-oxa-3,12,20,31,32-pentazahexacyclo[24.3.1.12,5.110,13.016,24.017,21]dotriaconta-1(30),2,4,10,12,16,18,21,23,26,28-undecaen-6-yl)phenyl]propanoic acid

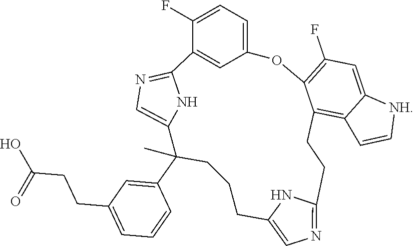 [SMILES: CC1(c2cccc(CCC(=O)O)c2)CCCc2cnc([nH]2)CCc2c(c(F)cc3[nH]ccc23)Oc2ccc(F)c(c2)-c2ncc1[nH]2]

Step B: The identical conditions described in Step I of Example 21 was used to prepare the title compound (6.2 mg, 21%) as a white solid. MS (ESI): 622 m/z [M+H]⁺, retention time: 1.33 minutes, purity: 95% (214 nm) (LC-MS method 6). ¹H NMR (400 MHz, CD₃OD) δ 7.76-7.70 (m, 2H), 7.46-7.42 (m, 1H), 7.36-7.72 (m, 2H), 7.27-7.28 (m, 3H), 7.26 (m, 2H), 7.14-7.12 (m, 1H), 6.69-6.64 (m, 1H), 3.32-3.33 (m, 2H), 2.88-2.92 (m, 4H), 2.58-2.60 (m, 2H), 1.95-2.1 (m, 2H), 1.71 (s, 3H), 1.28-1.41 (m, 4H) ppm.

Example 23. Compound 23. 3-[3-(23,29-Difluoro-6-methyl-25-oxa-31-thia-3,12,20,32-tetrazahexacyclo[24.3.1.12,5.110,13.016,24.017,21]dotriaconta-1(30),2,4,10,12,16,18,21,23,26,28-undecaen-6-yl)phenyl]propanoic acid

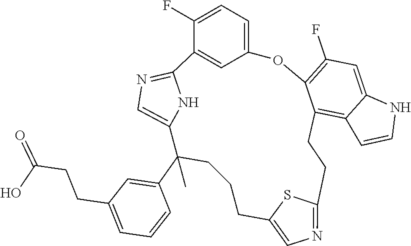 [SMILES: CC1(c2cccc(CCC(=O)O)c2)CCCC2=CN=C(CCc3c(c(F)cc4[nH]ccc34)Oc3ccc(F)c(c3)-c3ncc1[nH]3)S2]

Methyl 3-[3-(23,29-Difluoro-6-methyl-25-oxa-31-thia-3,12,20,32-tetrazahexacyclo-[24.3.1.12,5.110,13.016,24.017,21]dotriaconta-1(30),2,4,10,12,16,18,21,23,26,28-undecaen-6-yl)phenyl]propanoate

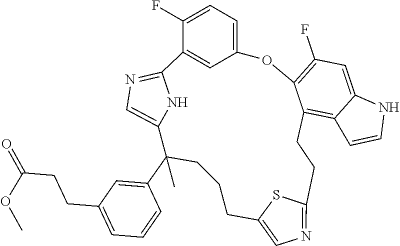 [SMILES: COC(=O)CCc1cccc(C2(C)CCCC3=CN=C(CCc4c(c(F)cc5[nH]ccc45)Oc4ccc(F)c(c4)-c4ncc2[nH]4)S3)c1]

Step A: To a stirred solution of methyl 3-[3-(23,29-difluoro-6-methyl-10,13-dioxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoate (Step G product of Example 21, 35 mg, 0.05 mmol) in 5 mL of tetrahydrofuran (5 ml) was added Lawson's reagent (30 mg, 0.1 mmol). The reaction was stirred at 70° C. for 4 hour under nitrogen. The mixture was partitioned between water (20 mL) and ethyl acetate (20 mL). The separated organic phase, combined with two additional ethyl acetate extracts, was dried over magnesium sulfate, and concentrated. The residue was purified by preparative thin layer chromatography to provide the title compound (17.5 mg, 50%) as yellow oil. MS (ESI): 653 m/z [M+H]⁺, retention time: 1.82 minutes, purity: 92% (214 nm) (LC-MS method 2).

Compound 23: 3-[3-(23,29-Difluoro-6-methyl-25-oxa-31-thia-3,12,20,32-tetrazahexacyclo[24.3.1.12,5.110,13.016,24.017,21]dotriaconta-1(30),2,4,10,12,16,18,21,23,26,28-undecaen-6-yl)phenyl]propanoic acid

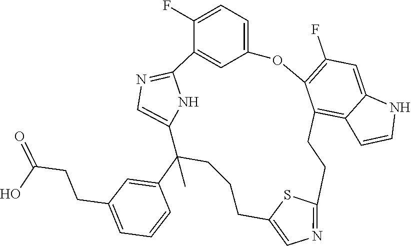 [SMILES: CC1(c2cccc(CCC(=O)O)c2)CCCC2=CN=C(CCc3c(c(F)cc4[nH]ccc34)Oc3ccc(F)c(c3)-c3ncc1[nH]3)S2]

Step B: The identical conditions described in Step I of Example 21 was used to prepare the title compound (12.2 mg, 70%) as a white solid. MS (ESI): 639 m/z [M+H]⁺, retention time: 1.73 minutes, purity: 95% (214 nm) (LC-MS method 2). ¹H NMR (400 MHz, CD₃OD) δ 7.77 (s, 1H), 7.55 (s, 1H), 7.36-7.38 (m, 2H), 7.29-7.33 (m, 1H), 7.26-7.28 (m, 2H), 7.10-7.24 (m, 4H), 6.69-6.65 (m, 1H), 3.32-3.33 (m, 1H), 2.79-2.98 (m, 3H), 2.57-2.89 (m, 2H), 12.52-2.58 (m, 2H), 2.07-2.05 (m, 2H), 1.63 (s, 3H), 1.28-1.41 (m, 4H) ppm.

Example 24. Compound 24. 3-[3-(24,30-Difluoro-9-hydroxy-6-methyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

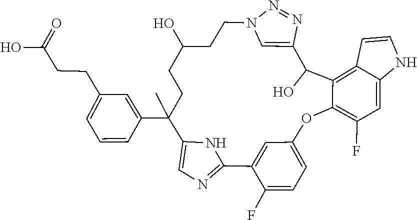 [SMILES: CC1(c2cccc(CCC(=O)O)c2)CCC(O)CCn2cc(nn2)C(O)c2c(c(F)cc3[nH]ccc23)Oc2ccc(F)c(c2)-c2ncc1[nH]2]

Exchanging 1-bromo-3-(3-iodophenyl)-3-methylnon-8-yn-2-one (Intermediate 2-51) with 10-bromo-8-(3-iodophenyl)-8-methyl-9-oxodec-1-yn-5-yl acetate (Intermediate 2-13, 2.5 g, 4.96 mmol), the reaction procedure sequence (Steps A to E) described for Example 2 was used to prepare the title compound (500 mg) as a white solid. MS (ESI): 653 m/z [M+H]⁺, retention time: 1.89 minutes, purity: >99% (214 nm) (LC-MS method 11) (mixture of 4 diastereomers).

Example 25. Compound 24-2. Methyl 3-[3-(24,30-Difluoro-9-hydroxy-6-methyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoate

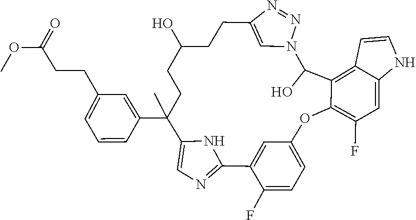 [SMILES: COC(=O)CCc1cccc(C2(C)CCC(O)CCc3cn(nn3)C(O)c3c(c(F)cc4[nH]ccc34)Oc3ccc(F)c(c3)-c3ncc2[nH]3)c1]

To a stirred solution of Example 24 (100 mg, 0.153 mmol) in methanol (6 mL) was added two drops of concentrated sulfuric acid. The mixture was stirred at room temperature for 16 hours and concentrated. The residue was treated with saturated sodium bicarbonate and the pH was adjusted to ˜7. The formed precipitate was collected by filtration to afford the title compound (92.0 mg, 90.1%) as white solid. MS (ESI): 667 m/z [M+H]⁺, retention time: 2.06 minutes, purity: >99% (214 nm) (LC-MS method 2). ¹H NMR (400 MHz, CD₃OD) d 7.54-7.34 (m, 6H), 7.22 (t, J=8.0 Hz, 1H), 7.11-7.04 (m, 3H), 6.78 (s, 1H), 6.58-6.53 (m, 1H), 5.97-5.79 (m, 2H), 4.88-4.87 (m, 1H), 3.67 (s, 3H), 2.86 (t, J=7.2 Hz, 2H), 2.63-2.29 (m, 5H), 2.02-1.28 (m, 8H) ppm

Example 26. Compound 26. 3-[3-(24,30-Difluoro-6-methyl-9-oxo-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

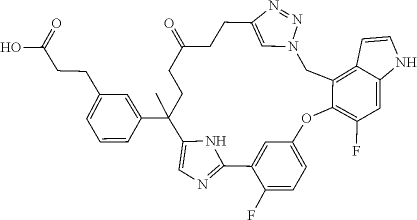 [SMILES: CC1(c2cccc(CCC(=O)O)c2)CCC(=O)CCc2cn(nn2)Cc2c(c(F)cc3[nH]ccc23)Oc2ccc(F)c(c2)-c2ncc1[nH]2]

To a stirred solution of 3-[3-(24,30-Difluoro-9-hydroxy-6-methyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid (Example 24, 115 mg, 0.18 mmol) in dimethyl sulfoxide (6 mL) was added 2-iodoxybenzoic acid (148 mg, 0.53 mmol). The reaction was stirred at room temperature for 16 hours, then partitioned between brine (25 mL) and ethyl acetate (50 mL). The separated organic phase was dried over sodium sulfate, and concentrated. The residue was purified by prep-HPLC to give the title compound (27.7 mg, 24%) as a yellow oil. MS (ESI): 651 m/z [M+H]⁺, retention time: 1.65 minutes, purity: >99% (214 nm) (LC-MS Method 9). ¹H NMR (400 MHz, CD₃OD) δ 7.45 (s, 1H), 7.39 (d, J=3.2 Hz, 1H), 7.34 (d, J=10.4 Hz, 1H), 7.22-7.02 (m, 8H), 6.61 (d, J=3.2 Hz, 1H), 5.88-5.80 (m, 2H), 2.85 (t, J=7.6 Hz, 2H), 2.69-2.41 (m, 8H), 2.28-2.17 (m, 2H), 1.52 (s, 3H) ppm.

Example 27. Compound 27A, 27B, and 27C. Diastereomers 1,2 and 3 of 3-[3-(24,30-difluoro-11-methyl-10,26-dioxa-3,13,14,15,21,33-hexazahexacyclo-[25.3.1.12,5.112,15.017,25.018,22]tritriaconta-1(31),2,4,12(32),13,17,19,22,24,27,29-undecaen-6-yl)-2-fluoro-phenyl]propanoic acid

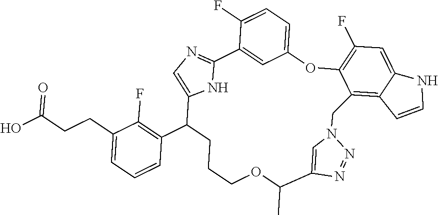 [SMILES: CC1OCCCC(c2cccc(CCC(=O)O)c2F)c2cnc([nH]2)-c2cc(ccc2F)Oc2c(F)cc3[nH]ccc3c2Cn2cc1nn2]

Exchanging 1-bromo-3-(3-iodophenyl)-3-methylnon-8-yn-2-one (Intermediate 2-51) with 1-bromo-3-(3-bromo-2-fluorophenyl)-6-(but-3-yn-2-yloxy)hexan-2-one (Intermediate 2-14, 1.74 g, 4.16 mmol), the reaction procedure sequence (Steps A to E) described for Example 2 was used to prepare the racemic title compounds (450 mg, a mixture of 4 diastereomers originated from 2 chiral centers) as a white solid. The two pairs of the diastereomers were separated by prep-HPLC to give Peak 1 (first peak, 200 mg, 44%) and Peak 2 (second peak, 194 mg, 43%). For Peak 2, the two enantiomers were not separated and designated as Compound 27C (diastereomer 3). Peak 1 was subject to chiral HPLC separation under the following condition: Instrument: Gilson-281; Column: OZ 20*250 mm, 10 μm; Mobile Phase: n-Hexane (0.1% diethylamine): ethanol (0.1% diethylamine)=65:35; Run time per injection: 15 minutes; Injection: 0.8 mL; Sample solution: 130 mg in 5 mL methanol. The first eluent, Diastereomer 1, was designated as Compound 27A (53 mg, 27%) and the second eluent, Diastereomer 2, was designated as Compound 27B (58 mg, 29%).

Compound 27C: MS (ESI): 657 m/z [M+H]⁺, retention time: 1.67 minutes, purity: 95% (214 nm) (LC-MS method 2). ¹H NMR (400 MHz, CD₃OD) δ 7.67 (s, 1H), 7.45-7.05 (m, 7H), 6.97 (t, J=7.6 Hz, 1H), 6.78 (s, 1H), 6.17 (d, J=3.2 Hz, 1H), 6.00 (d, J=15.2 Hz, 1H) 5.92 (d, J=15.2 Hz, 1H), 4.48 (q, J=6.4 Hz, 1H), 4.26-4.21 (m, 1H), 3.60-3.49 (m, 1H), 3.18-3.10 (m, 1H), 2.92 (t, J=7.6 Hz, 2H), 2.55 (t, J=7.6 Hz, 2H), 2.33-2.25 (m, 1H), 1.85-1.75 (m, 1H), 1.65-1.30 (m, 5H) ppm.

Compound 27A: MS (ESI): 657 m/z [M+H]⁺, retention time: 1.67 minutes, purity: 98% (214 nm) (LC-MS method 2). ¹H NMR (400 MHz, CD₃OD) δ 7.61 (s, 1H), 7.42-6.80 (m, 9H), 6.35 (s, 1H), 6.03-5.88 (m, 2H), 4.37 (q, J=6.4 Hz, 1H), 4.24-4.17 (m, 1H), 3.35-3.30 (m, 2H), 2.91 (t, J=7.6 Hz, 2H), 2.54 (t, J=7.6 Hz, 2H), 2.35-2.25 (m, 1H), 1.77-1.67 (m, 1H), 1.64-1.47 (m, 2H), 1.39 (d, J=6.4 Hz, 3H) ppm.

Compound 27B: MS (ESI): 657 m/z [M+H]⁺, retention time: 1.67 minutes, purity: 98% (214 nm) (LC-MS method 2). ¹H NMR (400 MHz, CD₃OD) δ 7.61 (s, 1H), 7.42-6.80 (m, 7H), 6.93 (t, J=7.6 Hz, 1H), 6.86 (s, 1H), 6.35 (s, 1H), 6.03-5.88 (m, 2H), 4.37 (q, J=6.4 Hz, 1H), 4.24-4.17 (m, 1H), 3.35-3.30 (m, 2H), 2.91 (t, J=7.6 Hz, 2H), 2.54 (t, J=7.6 Hz, 2H), 2.35-2.25 (m, 1H), 1.77-1.67 (m, 1H), 1.64-1.30 (m, 5H) ppm

Example 28. Compound 28. 3-[3-(24,30-Difluoro-11,11-dimethyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)-2-fluoro-phenyl]propanoic acid

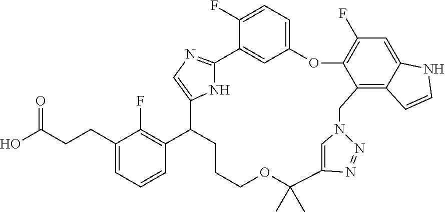 [SMILES: CC1(C)OCCCC(c2cccc(CCC(=O)O)c2F)c2cnc([nH]2)-c2cc(ccc2F)Oc2c(F)cc3[nH]ccc3c2Cn2cc1nn2]

Exchanging 1-bromo-3-(3-iodophenyl)-3-methylnon-8-yn-2-one (Intermediate 2-51) with 1-bromo-3-(3-bromo-2-fluorophenyl)-6-((2-methylbut-3-yn-2-yl)oxy)hexan-2-one (Intermediate 2-15, 1.0 g, 2.2 mmol), the reaction procedure sequence (Steps A to E) described for Example 2 was used to prepare the racemic title compound (15.4 mg) as a white solid. MS (ESI): 671 m/z [M+H]⁺, retention time: 1.68 minutes, purity: >99% (214 nm) (LC-MS method 2). ¹H NMR (400 MHz, CD₃OD) δ 7.60 (s, 1H), 7.39-7.25 (m, 5H), 7.16 (t, J=7.6 Hz, 1H), 7.08 (t, J=7.2 Hz, 1H), 6.95 (t, J=7.6 Hz, 1H), 6.84 (s, 1H), 6.22 (s, 1H), 6.05-5.90 (m, 2H), 4.22-4.18 (m, 1H), 3.22-3.17 (m, 1H), 3.11-2.98 (m, 1H), 2.92 (t, J=7.6 Hz, 2H), 2.56 (t, J=7.6 Hz, 2H), 2.25-2.18 (m, 1H), 1.78-1.70 (m, 1H), 1.48-1.24 (m, 8H) ppm.

Example 29. Compound 29A and Compound 29B. 3-[3-(24,30-Difluoro-6-methyl-26-oxa-3,13,14,15,21,32,33-heptazahexacyclo-[25.3.1.12,5.112,15.017,25.018,22]tritriaconta-1(31),2,4,12(32),13,17,19,22,24,27,29-undecaen-6-yl)phenyl]propanoic acid and 3-[3-(25,31-Difluoro-6-methyl-27-oxa-3,13,14,15,16,22,33-heptazahexacyclo-[26.3.1.12,5.012,16.018,26.019,23]tritriaconta-1(32),2,4,12,14,18,20,23,25,28,30-undecaen-6-yl)phenyl]propanoic acid

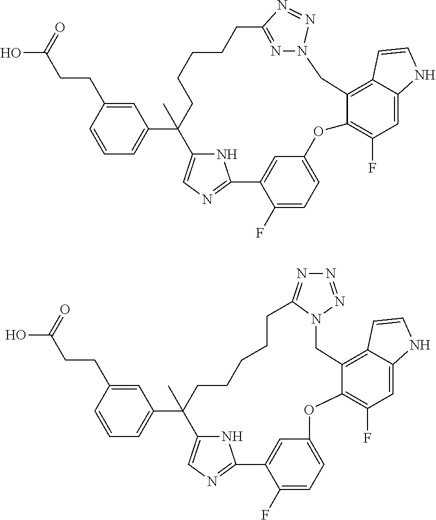 [SMILES: CC1(c2cccc(CCC(=O)O)c2)CCCCCc2nnn(n2)Cc2c(c(F)cc3[nH]ccc23)Oc2ccc(F)c(c2)-c2ncc1[nH]2 ; CC1(c2cccc(CCC(=O)O)c2)CCCCCc2nnnn2Cc2c(c(F)cc3[nH]ccc23)Oc2ccc(F)c(c2)-c2ncc1[nH]2]

Ethyl 3-(3-(7-cyano-2-(2-(2-fluoro-5-((6-fluoro-4-(hydroxymethyl)-1H-indol-5-yl)oxy)phenyl)-1H-imidazol-5-yl)heptan-2-yl)phenyl)propanoate

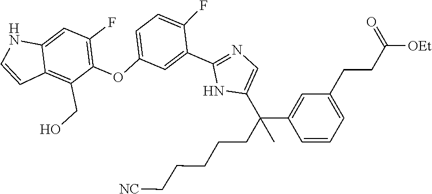 [SMILES: CCOC(=O)CCc1cccc(C(C)(CCCCCC#N)c2cnc(-c3cc(Oc4c(F)cc5[nH]ccc5c4CO)ccc3F)[nH]2)c1]

Step A: To a stirred solution of 2-fluoro-5-((6-fluoro-4-(hydroxymethyl)-1H-indol-5-yl)oxy)-benzimidamide (Intermediate 6-6, 1.57 g, 4.95 mmol) in dimethylformamide (50 mL) was added ethyl 3-(3-(1-bromo-8-cyano-3-methyl-2-oxooctan-3-yl)phenyl)propanoate (Intermediate 2-16, 1.9 g, 4.5 mmol) and sodium bicarbonate (760 mg, 9.05 mmol). The reaction was stirred at room temperature for 1 hour, then the temperature raised to 90° C. and stirred for another 3 hours. The mixture was cooled to room temperature, quenched with water (150 mL), and extracted with ethyl acetate (3×50 mL). The combined organic extracts were washed with brine (50 mL), dried over sodium sulfate, and concentrated. The residue was purified by automated flash chromatography (40 g silica gel column, eluting with 0-100% ethyl acetate in petroleum ether) to give the title compound (1.8 g, yield 62%) as a yellow solid. MS (ESI): 641 m/z [M+H]⁺, retention time: 1.60 minutes, purity: 90% (254 nm) (LC-MS method 2). 24,30-Difluoro-6-methyl-6-[5-(2H-tetrazol-5-yl)pentyl]-15,26-dioxa-3,21,33-triazahexacyclo-[25.3.1.12,5.17,11.017,25.018,22]tritriaconta-1(31),2,4,7(32),8,10,17,19,22,24,27,29-dodecaen-14-one

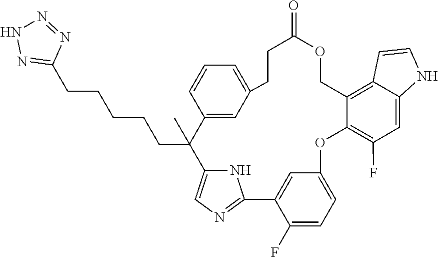 [SMILES: CC1(CCCCCc2nn[nH]n2)c2cccc(c2)CCC(=O)OCc2c(c(F)cc3[nH]ccc23)Oc2ccc(F)c(c2)-c2ncc1[nH]2]

Step B: To a stirred solution of Step A product (0.28 g, 0.437 mmol) in toluene (15 mL) was added azido(tributyl)stannane (0.58 g, 1.75 mmol). The mixture was refluxed for 3 days. The solvent was evaporated and the residue was purified by automated flash chromatography (12 g silica gel column, eluting with 0-10% methanol in dichloromethane) to give the title compound (170 mg, yield 61%) as a solid. MS (ESI): 638 m/z [M+H]⁺, retention time: 1.41 minutes, purity: 90% (254 nm) (LC-MS method 2).

Ethyl 3-(3-(2-(2-(2-fluoro-5-((6-fluoro-4-(hydroxymethyl)-1H-indol-5-yl)oxy)phenyl)-1H-imidazol-5-yl)-7-(2H-tetrazol-5-yl)heptan-2-yl)phenyl)propanoate

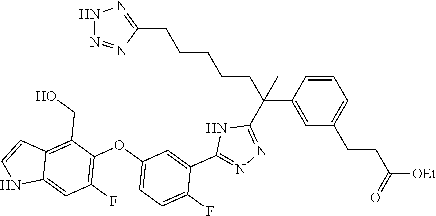 [SMILES: CCOC(=O)CCc1cccc(C(C)(CCCCCc2nn[nH]n2)c2nnc(-c3cc(Oc4c(F)cc5[nH]ccc5c4CO)ccc3F)[nH]2)c1]

Step C: To a stirred solution of Step B product (275 mg, 0.4 mmol) in ethanol (10 mL) was added 50 μL of concentrate sulfuric acid. The reaction was stirred at 70° C. for 4 hours. LC-MS monitoring indicated formation of desired product, with an unknown impurity. Potassium carbonate (596 mg) was added to the reaction system and stirring continued at 50° C. overnight. The reaction solution was diluted with ethyl acetate (50 mL), washed with brine, dried over sodium sulfate, and concentrated. The residue was purified by automated flash chromatography (4 g silica gel column, eluting with 0-10% methanol in dichloromethane) to give the title compound (105 mg, 38%) as a solid. MS (ESI): 684 m/z [M+H]⁺, retention time: 1.41 minutes, purity: 91% (254 nm) (LC-MS method 12).

Compounds 29A and 29B: Regio isomers ! and 2 of 3-[3-(24,30-Difluoro-6-methyl-26-oxa-3,13,14,15,21,32,33-heptazahexacyclo-[25.3.1.12,5.112,15.017,25.018,22]tritriaconta-1(31),2,4,12(32),13,17,19,22,24,27,29-undecaen-6-yl)phenyl]propanoic acid and 3-[3-(25,31-Difluoro-6-methyl-27-oxa-3,13,14,15,16,22,33-heptazahexacyclo-[26.3.1.12,5.012,16.018,26.019,23]tritriaconta-1(32),2,4,12,14,18,20,23,25,28,30-undecaen-6-yl)phenyl]propanoic acid (Definitive Regio-Isomer Chemistry Undetermined)

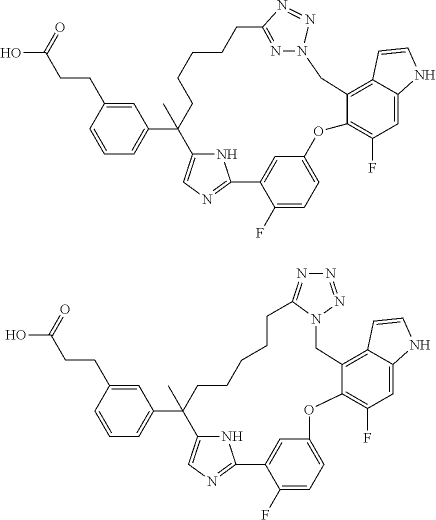 [SMILES: CC1(c2cccc(CCC(=O)O)c2)CCCCCc2nnn(n2)Cc2c(c(F)cc3[nH]ccc23)Oc2ccc(F)c(c2)-c2ncc1[nH]2 ; CC1(c2cccc(CCC(=O)O)c2)CCCCCc2nnnn2Cc2c(c(F)cc3[nH]ccc23)Oc2ccc(F)c(c2)-c2ncc1[nH]2]

Step D: In glove box, to a stirred solution of Step C product (105 mg, 0.15 mmol) in tetrahydrofuran (15 mL) was added triphenyl phosphine (81 mg, 0.31 mmol) and di-isopropyl azodicarboxylate (62 mg). The reaction was stirred at room temperature overnight, then diluted in ethyl acetate (50 mL). The mixture was washed with brine, dried over sodium sulfate, and concentrated. The residue was purified by automated flash chromatography (20 g silica gel column, eluting with 0-50% ethyl acetate in petroleum ether). The first eluent, the regio-isomer 1 (10 mg), combined with additional 22 mg from another run of the same sequence (total 32 mg), was further hydrolyzed, and purified by prep-HPLC to Compound 29A (5.8 mg, 19%); The second eluent, the regio-isomer 2 (7 mg), was combined with an additional 16 mg from another run of the same reaction sequence (total 23 mg), was further hydrolyzed, and purified by prep-HPLC to Compound 29B (9.9 mg, 45%). Both hydrolyses followed the Step E condition of Example 2. The absolute regio-chemistry structures of Compound 29A and Compound 29B was not determined.

Compound 29A: MS (ESI): 638 m/z [M+H]⁺, retention time: 1.72 minutes, purity: >99% (214 nm) (LC-MS method 2). ¹H NMR (400 MHz, CD₃OD) δ 7.32-7.28 (m, 2H), 7.21-7.19 (m, 2H), 7.14-7.06 (m, 3H), 7.04-6.98 (m, 3H), 6.55 (d, J=3.2 Hz, 1H), 6.13-6.04 (m, 2H), 2.85-2.81 (m, 2H), 2.50-2.42 (m, 4H), 2.13-1.97 (m, 2H), 1.63-1.57 (m, 2H), 1.49 (s, 3H), 1.47-1.36 (m, 2H), 1.19-0.93 (m, 2H) ppm.

Compound 29B: MS (ESI): 638 m/z [M+H]⁺, retention time: 1.66 minutes, purity: >99% (214 nm) (LC-MS method 2). ¹H NMR (400 MHz, CD₃OD) δ 7.38-7.34 (m, 2H), 7.22-7.10 (m, 4H), 7.00 (d, J=7.7 Hz, 2H), 6.94-6.91 (m, 2H), 6.02-5.89 (m, 3H), 2.84 (t, J=7.8 Hz, 2H), 2.65-2.45 (m, 4H), 2.17-2.13 (m, 1H), 1.82-1.76 (m, 1H), 1.51 (s, 3H), 1.49-1.42 (m, 2H), 1.35-0.94 (m, 4H) ppm.

Example 30. Compound 30A and Compound 30B 3-[3-(26,32-Difluoro-28-oxa-3,13,14,15,23,34,35-heptazahexacyclo[27.3.1.12,5.112,15.019,27.020,24]pentatriaconta-1(33),2,4,12(34),13,19,21,24,26,29,31-undecaen-6-yl)phenyl]propanoic acid and 3-[3-(27,33-difluoro-29-oxa-3,13,14,15,16,24,35-heptazahexacyclo[28.3.1.12,5.012,16.020,28.021,25]-pentatriaconta-1(34),2,4,12,14,20,22,25,27,30,32-undecaen-6-yl)phenyl]propanoic acid (Regio-Chemistry was not determined)

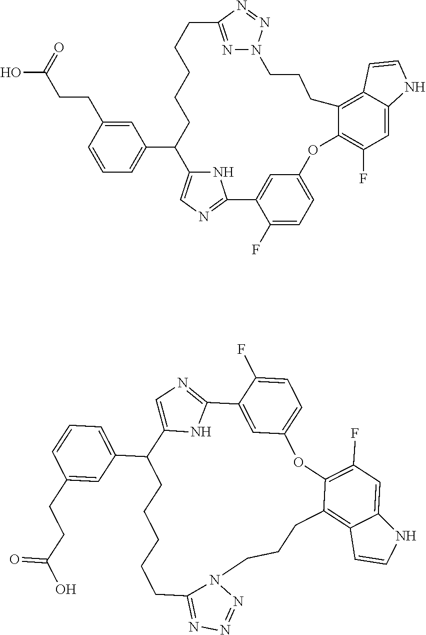 [SMILES: O=C(O)CCc1cccc(C2CCCCCc3nnn(n3)CCCc3c(c(F)cc4[nH]ccc34)Oc3ccc(F)c(c3)-c3ncc2[nH]3)c1 ; O=C(O)CCc1cccc(C2CCCCCc3nnnn3CCCc3c(c(F)cc4[nH]ccc34)Oc3ccc(F)c(c3)-c3ncc2[nH]3)c1]

Exchanging 2-fluoro-5-((6-fluoro-4-(hydroxymethyl)-1H-indol-5-yl)oxy)-benzimidamide (Intermediate 6-6) with 2-fluoro-5-((6-fluoro-4-(3-hydroxypropyl)-1H-indol-5-yl)oxy)-benzimidamide (Intermediate 6-3, 0.965 g, 2.8 mmol) and ethyl 3-(3-(1-bromo-8-cyano-3-methyl-2-oxooctan-3-yl)phenyl)propanoate (Intermediate 2-16) with ethyl 3-(3-(1-bromo-8-cyano-2-oxooctan-3-yl)phenyl)propanoate (Intermediate 2-2, 0.95 g, 2.33 mmol), the reaction procedure sequence (Steps A to D) described for Example 29A and 29B was used to prepare the two regio-isomers of title compounds. The two regio-isomers were separated at corresponding D as esters. The first eluent, Regio-isomer 1 (13 mg) was hydrolyzed to Compound 30A (10.6 mg, 85%); The second eluent, Regio-isomer 2 (6 mg) was hydrolyzed to Compound 30B (4.6 mg, 80%). The definitive regio-isomer chemistry was not determined.

Compound 30A: MS (ESI): 652 m/z [M+H]⁺, retention time: 1.43 minutes, purity: 97% (214 nm) (LC-MS method 12). ¹H NMR (400 MHz, CD₃OD) δ 7.42 (dd, J=6.1, 3.1 Hz, 1H), 7.21 (d, J=3.2 Hz, 1H), 7.13-6.90 (m, 6H), 6.83 (s, 1H), 6.61-6.54 (m, 1H), 6.50 (t, J=4.7 Hz, 1H), 4.49 (t, J=6.9 Hz, 2H), 3.88-3.83 (m, 1H), 2.87-2.72 (m, 4H), 2.67-2.58 (m, 2H), 2.45 (t, J=7.7 Hz, 2H), 2.31-2.16 (m, 2H), 2.10 (d, J=8.3 Hz, 1H), 1.78-1.74 (m, 1H), 1.71-1.66 (m, 2H), 1.33-1.27 (m, 4H) ppm.

Compound 30B: MS (ESI): 652 m/z [M+H]⁺, retention time: 1.38 minutes, purity: 94% (214 nm) (LC-MS method 12). ¹H NMR (400 MHz, CD₃OD) δ 7.52 (dd, J=5.8, 3.1 Hz, 1H), 7.16 (d, J=3.2 Hz, 1H), 7.13-6.92 (m, 6H), 6.86 (dd, J=8.1, 4.4 Hz, 1H), 6.62 (s, 1H), 6.15 (d, J=2.6 Hz, 1H), 4.14 (t, J=7.4 Hz, 2H), 3.86-3.80 (m, 1H), 2.96-2.87 (m, 2H), 2.79 (t, J=7.6 Hz, 2H), 2.61-2.40 (m, 4H), 2.27-1.85 (m, 4H), 1.83-1.53 (m, 6H) ppm.

Example 31. Compound 31. 3-[2-Fluoro-3-(11,11,24,30-tetrafluoro-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

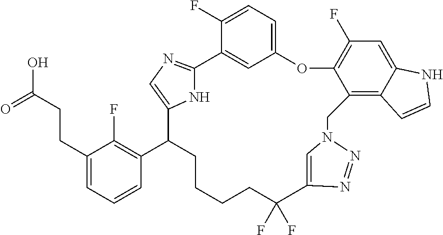 [SMILES: O=C(O)CCc1cccc(C2CCCCC(F)(F)c3cn(nn3)Cc3c(c(F)cc4[nH]ccc34)Oc3ccc(F)c(c3)-c3ncc2[nH]3)c1F]

Exchanging 1-bromo-3-(3-iodophenyl)-3-methylnon-8-yn-2-one (Intermediate 2-51) with 1-bromo-3-(3-bromo-2-fluorophenyl)-8,8-difluorodec-9-yn-2-one (Intermediate 2-17, 650 mg, 1.4 mmol), the reaction procedure sequence (Steps A to E) described for Example 2 was used to prepare the title compound (29.4 mg) as a white solid. MS (ESI): 677 m/z [M+H]⁺, retention time: 1.42 minutes, purity: >99% (254 nm) (LC-MS method 5). 1H NMR (400 MHz, CD₃OD) δ 7.96 (s, 1H), 7.46 (d, J=2.4 Hz, 1H), 7.37 (d, J=10.7 Hz, 1H), 7.32-7.23 (m, 2H), 7.19-6.90 (m, 5H), 6.85 (s, 1H), 6.09-5.91 (m, 2H), 4.18 (d, J=9.1 Hz, 1H), 2.90 (t, J=7.8 Hz, 2H), 2.54 (t, J=7.6 Hz, 2H), 2.19-1.95 (m, 3H), 1.87-1.78 (m, 1H), 1.45-1.06 (m, 4H) ppm.

Example 32. Compound 32. 3-[3-(24,30-Difluoro-6,11,11-trimethyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

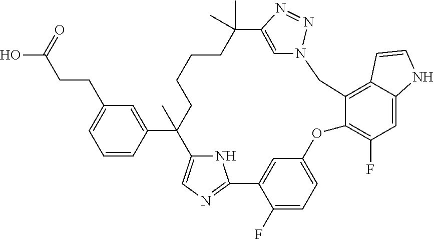 [SMILES: CC1(C)CCCCC(C)(c2cccc(CCC(=O)O)c2)c2cnc([nH]2)-c2cc(ccc2F)Oc2c(F)cc3[nH]ccc3c2Cn2cc1nn2]

Exchanging 1-bromo-3-(3-iodophenyl)-3-methylnon-8-yn-2-one (Intermediate 2-51) with 1-bromo-3-(3-iodophenyl)-3,8,8-trimethyldec-9-yn-2-one (Intermediate 2-18, 875 mg, 1.84 mmol), the reaction procedure sequence (Steps A to E) described for Example 2 was used to prepare the title compound (106 mg) as a white solid. MS (ESI): 665 m/z [M+H]⁺, retention time: 1.76 minutes, purity: >99% (214 nm) (LC-MS method 2). ¹H NMR (400 MHz, CD₃OD) δ 7.39-7.26 (m, 5H), 7.14-7.06 (m, 4H), 6.99 (t, J=6.9 Hz, 2H), 6.64 (d, J=2.8 Hz, 1H), 5.95 (d, J=14.4 Hz, 1H), 5.77 (d, J=14.3 Hz, 1H), 2.83 (t, J=7.8 Hz, 2H), 2.51 (t, J=7.8 Hz, 2H), 2.02 (t, J=7.1 Hz, 2H), 1.46-1.40 (m, 5H), 1.26-1.00 (m, 8H), 0.83-0.56 (m, 2H) ppm.

Example 33. Compound 33. 3-[3-(24,30-difluoro-11,11-dimethyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)-2-fluoro-phenyl]propanoic acid

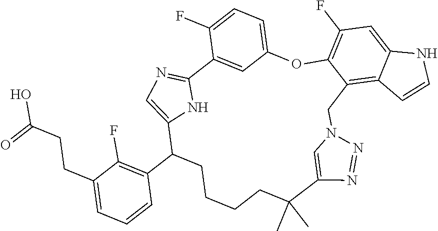 [SMILES: CC1(C)CCCCC(c2cccc(CCC(=O)O)c2F)c2cnc([nH]2)-c2cc(ccc2F)Oc2c(F)cc3[nH]ccc3c2Cn2cc1nn2]

Exchanging 1-bromo-3-(3-iodophenyl)-3-methylnon-8-yn-2-one (Intermediate 2-51) with 1-bromo-3-(3-bromo-2-fluorophenyl)-8,8-dimethyldec-9-yn-2-one (Intermediate 2-19, 587 mg, 1.31 mmol), the reaction procedure sequence (Steps A to E) described for Example 2 was used to prepare the title compound (15 mg) as a white solid. MS (ESI): 669 m/z [M+H]⁺, retention time: 1.77 minutes, purity: >99% (214 nm) (LC-MS method 2). ¹H NMR (400 MHz, CD₃OD) δ 7.42-7.27 (m, 5H), 7.17-7.04 (m, 3H), 6.96-6.90 (m, 2H), 6.74 (d, J=2.8 Hz, 1H), 5.95-5.85 (m, 2H), 4.16 (d, J=8.6 Hz, 1H), 2.54-2.50 (m, 2H), 2.91-2.88 (t, 2H), 2.04-1.72 (m, 2H), 1.56-1.23 (m, 4H), 1.11 (s, 3H), 1.06 (s, 3H), 0.97-0.83 (m, 2H) ppm.

Example 34. Compound 34. 3-[3-(24,30-Difluoro-6-methyl-26,32-dioxa-3,14,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

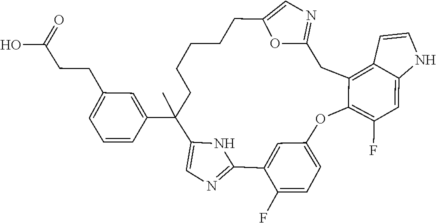 [SMILES: CC1(c2cccc(CCC(=O)O)c2)CCCCCC2=CN=C(Cc3c(c(F)cc4[nH]ccc34)Oc3ccc(F)c(c3)-c3ncc1[nH]3)O2]

Exchanging 1-azido-8-bromo-6-(3-iodophenyl)-6-methyl-7-oxooctan-2-yl acetate (Intermediate 2-12) with 1-azido-10-bromo-8-(3-iodophenyl)-8-methyl-9-oxodecan-2-yl acetate (Intermediate 2-20, 2.4 g, 4.35 mmol) and methyl (E)-3-(5-(3-carbamimidoyl-4-fluorophenoxy)-6-fluoro-1H-indol-4-yl)acrylate (Intermediate 6-5) with ethyl 2-(5-(3-carbamimidoyl-4-fluorophenoxy)-6-fluoro-1H-indol-4-yl)acetate (Intermediate 29, 1.6 g, 4.35 mmol), the reaction procedure sequence (Steps A to I) described for Example 21 was used to prepare the title compound (16.5 mg) as a white solid. MS (ESI): 637 m/z [M+H]⁺, retention time: 1.73 minutes, purity: 97% (214 nm) (LC-MS method 2). ¹H NMR (400 MHz, CD₃OD) δ 7.26-6.85 (m, 10H), 6.42 (s, 1H), 6.33 (s, 1H), 4.26-4.09 (m, 2H), 2.74 (t, J=7.6 Hz, 2H), 2.42 (t, J=7.6 Hz, 2H), 2.13-1.87 (m, 4H), 1.44-1.15 (m, 9H) ppm.

Example 35. Compound 35. 3-[3-(24,30-Difluoro-6-methyl-26-oxa-32-thia-3,14,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

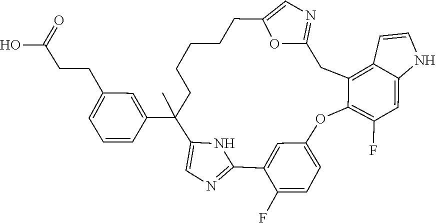 [SMILES: CC1(c2cccc(CCC(=O)O)c2)CCCCCC2=CN=C(Cc3c(c(F)cc4[nH]ccc34)Oc3ccc(F)c(c3)-c3ncc1[nH]3)O2]

Exchanging 1-azido-8-bromo-6-(3-iodophenyl)-6-methyl-7-oxooctan-2-yl acetate (Intermediate 2-12) with 1-azido-10-bromo-8-(3-iodophenyl)-8-methyl-9-oxodecan-2-yl acetate (Intermediate 2-20, 2.4 g, 4.35 mmol) and methyl (E)-3-(5-(3-carbamimidoyl-4-fluorophenoxy)-6-fluoro-1H-indol-4-yl)acrylate (Intermediate 6-5) with ethyl 2-(5-(3-carbamimidoyl-4-fluorophenoxy)-6-fluoro-1H-indol-4-yl)acetate (Intermediate 29, 1.6 g, 4.35 mmol), the reaction procedure sequence (Steps A to G) described for Example 21 was used to prepare ethyl 3-[3-(24,30-difluoro-6-methyl-12,15-dioxo-26-oxa-3,14,21,32-tetrazapentacyclo[25.3.1.12,5.017,25.018,22]-dotriaconta-1(31),2,4,17,19,22,24,27,29-nonaen-6-yl)phenyl]-propanoate (100 mg); 40 mg was taken out of the 100 mg and subjected to the reaction sequence (Step A and B) described for Example 23, to prepare the title compound (3.2 mg) as a white solid. MS (ESI): 653 m/z [M+H]⁺, retention time: 1.83 minutes, purity: 97% (214 nm) (LC-MS method 2). ¹H NMR (400 MHz, CD₃OD) δ 7.30-6.71 (m, 11H), 6.32 (d, J=2.6 Hz, 1H), 4.47-4.35 (m, 2H), 2.73 (t, J=7.7 Hz, 2H), 2.52-2.27 (m, 4H), 1.94 (s, 2H), 1.53-1.09 (m, 9H) ppm.

Example 36. Compound 36. 3-[3-(24,30-Difluoro-6-methyl-26-oxa-14-thia-3,21,32,33-tetrazahexacyclo[25.3.1.12,5.112,15.017,25.018,22]tritriaconta-1(31),2,4,12,15(32),17,19,22,24,27,29-undecaen-6-yl)phenyl]propanoic acid

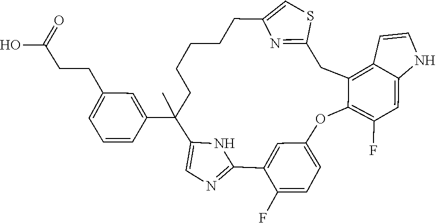 [SMILES: CC1(c2cccc(CCC(=O)O)c2)CCCCCc2csc(n2)Cc2c(c(F)cc3[nH]ccc23)Oc2ccc(F)c(c2)-c2ncc1[nH]2]

Methyl (E)-3-(3-(2-(2-(5-((4-((4-bromothiazol-2-yl)methyl)-6-fluoro-1H-indol-5-yl)oxy)-2-fluorophenyl)-1H-imidazol-5-yl)hept-6-yn-2-yl)phenyl)acrylate

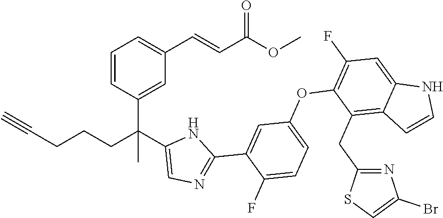 [SMILES: C#CCCCC(C)(c1cccc(/C=C/C(=O)OC)c1)c1cnc(-c2cc(Oc3c(F)cc4[nH]ccc4c3Cc3nc(Br)cs3)ccc2F)[nH]1]

Step A: To a stirred solution of 5-((4-((4-bromothiazol-2-yl)methyl)-6-fluoro-1H-indol-5-yl)oxy)-2-fluorobenzimidamide (Intermediate 30, 0.6 g, 1.3 mmol) and methyl (E)-3-(3-(1-bromo-3-methyl-2-oxooct-7-yn-3-yl)phenyl)acrylate (Intermediate 2-21, 0.45 g, 1.19 mmol) in dimethylformamide (10 mL) was added sodium bicarbonate (200 mg, 2.4 mmol). The mixture was stirred for 16 hours at 80° C., cooled to room temperature and quenched with water (50 mL). The solution was extracted with ethyl acetate (3×50 mL). The combined organic phases were washed with brine, dried over sodium sulfate, and concentrated. The residue was purified by column chromatography on silica gel, eluting with dichloromethane/methanol (95:5) to afford the title compound (0.45 g, 50%) as a solid. MS (ESI): 741, 743 m/z [M+H]⁺, retention time: 1.89 minutes, purity: 89% (254 nm) (LC-MS method 2).

Methyl (2E)-3-(3-(2-(2-(5-((4-((4-bromothiazol-2-yl)methyl)-6-fluoro-1H-indol-5-yl)oxy)-2-fluorophenyl)-1H-imidazol-5-yl)-7-(tributylstannyl)hept-6-en-2-yl)phenyl)acrylate

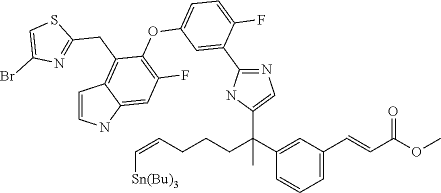

Step B: To a stirred and degassed solution of tris(dibenzylideneacetone)dipalladium(0) (6.2 mg, 0.0067 mmol) and tricyclohexylphosphine tetrafluoroborate (5 mg, 1.35e-5 mol) in dichloromethane (5 mL) was added diisopropylethylamine (8.7 mg, 0.0674 mmol). The mixture was stirred at room temperature for 10 minutes, then added Step A product (50 mg, 6.74e-5 mol), followed by a solution of tributylstannane (39 mg, 0.135 mmol) in dichloromethane (1 mL). The reaction mixture was stirred at room temperature for 30 minutes and concentrated. The residue was purified by automated flash column chromatography (4 g silica gel column, 0% to 5% methanol in dichloromethane) to afford the title compound (33 mg, 47%) as an oil. MS (ESI): no mass peak, retention time: 2.46 minutes, purity: 83% (214 nm) (LC-MS method 2).

Methyl (E)-3-[3-[(10E)-24,30-difluoro-6-methyl-26-oxa-14-thia-3,21,32,33-tetrazahexacyclo-[25.3.1.12,5.112,15.017,25.018,22]tritriaconta-1(31),2,4,10,12,15(32),17,19,22,24,27,29-dodecaen-6-yl]phenyl]prop-2-enoate

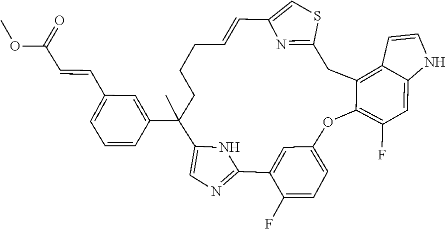 [SMILES: COC(=O)/C=C/c1cccc(C2(C)CCC/C=C/c3csc(n3)Cc3c(c(F)cc4[nH]ccc34)Oc3ccc(F)c(c3)-c3ncc2[nH]3)c1]

Step C: A seal tube was charged with the Step B product (300 mg, 0.29 mmol), Jackiephos (46 mg, 0.058 mmol), tris(dibenzylideneacetone)-dipalladium(0) (26.6 mg, 0.029 mmol) and toluene (150 mL) in a glove box. The red suspension was stirred for 16 hours at 50° C. in the glove box, then concentrated. The residue was purified by flash column chromatography (silica, 0%→5% methanol in dichloromethane) to afford the title compound (80 mg, 42%). MS (ESI): 663 m/z [M+H]⁺, retention time: 1.84 minutes, purity: >99% (254 nm) (LC-MS method 2).

Methyl 3-[3-(24,30-Difluoro-6-methyl-26,32-dioxa-3,14,21,33-tetrazahexacyclo-[25.3.1.12,5.112,15.017,25.018,22]tritriaconta-1(31),2,4,12,14,17,19,22,24,27,29-undecaen-6-yl)phenyl]propanoate

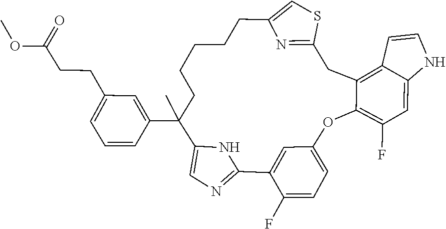 [SMILES: COC(=O)CCc1cccc(C2(C)CCCCCc3csc(n3)Cc3c(c(F)cc4[nH]ccc34)Oc3ccc(F)c(c3)-c3ncc2[nH]3)c1]

Step D: To a stirred solution of the Step C product (80 mg, 0.1 mmol) in tetrahydrofuran (10 mL) was added palladium on carbon (10%, 50% wet, 80 mg). The mixture was stirred under hydrogen atmosphere overnight, then filtered through a pad of Celite. The filtrate was concentrated. The residue was purified by prep-thin layer chromatography (dichloromethane\methanol=90\0) to give the title compound (20 mg, 24% yield) as a light-yellow solid. MS (ESI): 667 m/z [M+H]⁺, retention time: 1.82 minutes, purity: 71% (254 nm) (LC-MS method 2).

Compound 36: 3-[3-(24,30-Difluoro-6-methyl-26-oxa-14-thia-3,21,32,33-tetrazahexacyclo[25.3.1.12,5.112,15.017,25.018,22]tritriaconta-1(31),2,4,12,15(32),17,1922,24,27,29-undecaen-6-yl)henylpropanoic acid

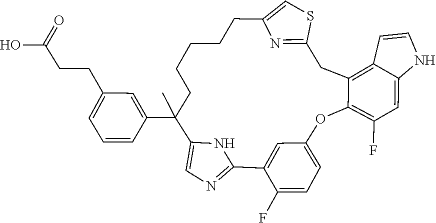 [SMILES: CC1(c2cccc(CCC(=O)O)c2)CCCCCc2csc(n2)Cc2c(c(F)cc3[nH]ccc23)Oc2ccc(F)c(c2)-c2ncc1[nH]2]

Step E: To a stirred solution of the Step D product (20 mg, 0.03 mmol) in methanol (3 mL) and water (1 mL) was added lithium hydroxide monohydrate (13 mg, 0.3 mmol). The mixture was stirred at room temperature for 3 hours, then adjusted pH to ˜4 with 1 N hydrochloric acid. The mixture was partitioned between water (15 mL) and ethyl acetate (20 mL). The separated organic layer was combined with two additional ethyl acetate extracts (2×20 mL). The combined organic phases were washed with brine, dried over sodium sulfate, and concentrated. The final residue was purified by prep-thin layer chromatography (dichloromethane\methanol=90\10) to give the title compound (10 mg, 51%). MS (ESI): 653 m/z [M+H]⁺, retention time: 1.77 minutes, purity: >99% (214 nm) (LC-MS method 2) as a white solid. ¹H NMR (400 MHz, CD₃OD) δ 8.38 (s, 1H), 7.31 (s, 1H), 7.21-6.87 (m, 9H), 6.74 (s, 1H), 6.24 (s, 1H), 4.24-4.16 (m, 2H), 2.75 (t, J=7.7 Hz, 2H), 2.40-2.44 (m, 4H), 2.15-2.25 (m, 1H), 1.84-1.76 (m, 1H), 1.48-1.40 (m, 5H), 1.10-1.30 (m, 4H) ppm.

Example 37. Compound 37. 3-[3-(24,30-Difluoro-6,9,9-trimethyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

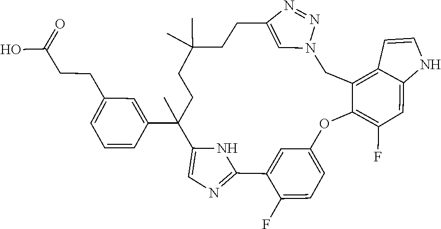 [SMILES: CC1(C)CCc2cn(nn2)Cc2c(c(F)cc3[nH]ccc23)Oc2ccc(F)c(c2)-c2ncc([nH]2)C(C)(c2cccc(CCC(=O)O)c2)CC1]

Exchanging 1-bromo-3-(3-iodophenyl)-3-methylnon-8-yn-2-one (Intermediate 2-51) with 1-bromo-3-(3-iodophenyl)-3,6,6-trimethyldec-9-yn-2-one (Intermediate 2-22, 1.67 g, 3.51 mmol), the reaction procedure sequence (Steps A to E) described for Example 3 was used to prepare the title compound (30 mg) as a white solid. MS (ESI): 665 m/z [M+H]⁺, retention time: 1.74 minutes, purity: 95% (214 nm) (LC-MS method 2). ¹H NMR (400 MHz, DMSO-d₆) δ 12.10 (s, 1H), 11.83 (s, 1H), 11.48 (s, 1H), 7.51-7.41 (m, 2H), 7.29-7.22 (m, 2H), 7.18-7.07 (m, 2H), 7.06-6.90 (m, 4H), 6.84 (dd, J=5.8, 3.2 Hz, 1H), 6.65 (s, 1H), 5.87-5.78 (m, 2H), 2.65 (t, J=7.6 Hz, 2H), 2.40 (t, J=7.7 Hz, 2H), 2.19-2.09 (m, 2H), 1.48 (s, 3H), 1.42-1.32 (m, 2H), 1.23 (m, 1H), 1.05 (s, 1H), 0.94-0.82 (m, 2H), 0.80-0.74 (m, 6H) ppm.

Example 38. Compound 38. 2-(Dimethylamino)ethyl 3-[3-(24,30-difluoro-6-methyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoate

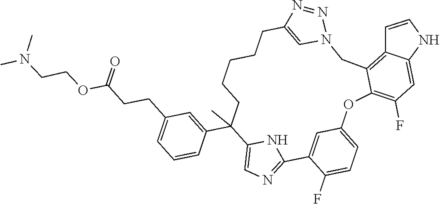 [SMILES: CN(C)CCOC(=O)CCc1cccc(C2(C)CCCCCc3cn(nn3)Cc3c(c(F)cc4[nH]ccc34)Oc3ccc(F)c(c3)-c3ncc2[nH]3)c1]

To a stirred solution of methyl 3-[3-(24,30-difluoro-6-methyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoate (Example 20, 250 mg, 0.384 mmol) in N,N-dimethylethanolamine (5 mL) was added potassium carbonate (159 mg, 3 eq). The reaction was stirred at 70° C. for 6 hours, cooled to room temperature. The mixture was diluted with water (20 mL) to form a precipitate. The precipitate was collected by filtration, and further purified by prep-thin layer chromatography to get the title compound (87 mg, 32%). MS (ESI): 708.4 m/z [M+H]⁺, retention time: 1.80 minutes, purity: >99% (214 nm) (LC-MS method 7).

Example 39. Compound 39A and Compound 39B. Enantiomers 1 and 2 of 2-morpholinoethyl 3-[3-(24,30-difluoro-6-methyl-26-oxa-3,13,14,15,21,33-hexazahexacyclo[25.3.1.12,5.112,15.017,25.018,22]-tritriaconta-1(31),2,4,12(32),13,17,19,22,24,27,29-undecaen-6-yl)phenyl]propanoate

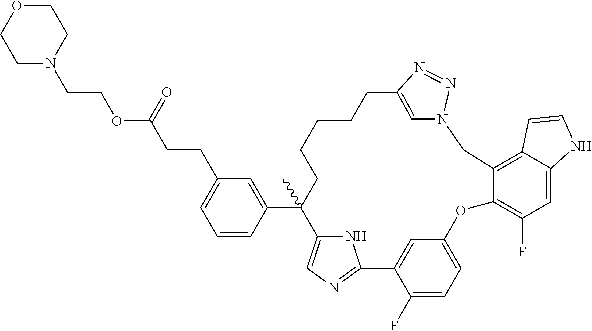 [SMILES: CC1(c2cccc(CCC(=O)OCCN3CCOCC3)c2)CCCCCc2cn(nn2)Cc2c(c(F)cc3[nH]ccc23)Oc2ccc(F)c(c2)-c2ncc1[nH]2]

To a stirred solution of methyl 3-[3-(24,30-difluoro-6-methyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoate (Example 20, 200 mg, 0.307 mmol) in N-(2-hydroxyethyl)morpholine (5 ml) was added potassium carbonate (127 mg, 3 eq). The reaction was stirred at 70° C. for 6 hours, cooled to room temperature and diluted with water (20 mL). The formed precipitate, the crude product, was collected by filtration. This crude product was purified by preparative thin layer chromatography to get the racemic title compound (126 mg, 55%). This racemic mixture was subject to chiral SFC separation under the following condition: Instrument: SFC-80 (Thar, Waters); Column: OJ 20*250 mm, 10 μm; Column temperature: 35° C.; Mobile phase: carbon dioxide/ethanol (1% 7 M methanol Ammonia as additive)=70/30; Flow rate: 80 g/minute; Back pressure: 100 bar; Detection wavelength: 214 nm; Cycle time: 4.5 minutes; Sample solution: 160 mg dissolved in 15 mL methanol. Injection volume: 0.6 mL. The first eluent, enantiomer 1 (60 mg), was designated as Compound 39A; The second eluent, enantiomer 2 (54 mg), was designated as Compound 39B.

Compound 39A: MS (ESI): 750 m/z [M+H]⁺, retention time: 1.75 minutes, purity: >99% (214 nm) (LC-MS method 7). ¹H NMR (400 MHz, CD₃OD) δ 7.37-7.27 (m, 3H), 7.23-7.08 (m, 4H), 7.04 (d, J=8.5 Hz, 3H), 6.96 (m, 1H), 6.53 (s, 1H), 5.87 (d, J=14.2 Hz, 1H), 5.75 (d, J=14.3 Hz, 1H), 4.12 (t, J=5.6 Hz, 2H), 3.77-3.53 (m, 4H), 2.81 (t, J=7.2 Hz, 2H), 2.43-2.54 (m, 4H), 2.48-2.40 (m, 4H), 1.90-2.30 (m, 4H), 1.46 (s, 3H), 1.42-1.19 (m, 4H), 1.14-1.08 (m, 1H), 0.90-0.87 (m, 1H) ppm.

Compound 39B: MS (ESI): 750 m/z [M+H]⁺, retention time: 1.75 minutes, purity: >99% (214 nm) (LC-MS method 7). ¹H NMR (400 MHz, CD₃OD) δ 7.37-7.27 (m, 3H), 7.23-7.08 (m, 4H), 7.04 (d, J=8.5 Hz, 3H), 6.96 (m, 1H), 6.53 (s, 1H), 5.87 (d, J=14.2 Hz, 1H), 5.75 (d, J=14.3 Hz, 1H), 4.12 (t, J=5.6 Hz, 2H), 3.77-3.54 (m, 4H), 2.81 (t, J=7.2 Hz, 2H), 2.43-2.54 (m, 4H), 2.48-2.40 (m, 4H), 1.90-2.30 (m, 4H), 1.46 (s, 3H), 1.42-1.20 (m, 4H), 1.14-1.09 (m, 1H), 0.90-0.87 (m, 1H) ppm.

Example 40. Compound 40. 3-[3-(24,30-Difluoro-11,11-dimethyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

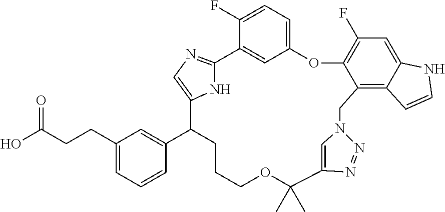 [SMILES: CC1(C)OCCCC(c2cccc(CCC(=O)O)c2)c2cnc([nH]2)-c2cc(ccc2F)Oc2c(F)cc3[nH]ccc3c2Cn2cc1nn2]

Exchanging 1-bromo-3-(3-iodophenyl)-3-methylnon-8-yn-2-one (Intermediate 2-51) with 1-bromo-3-(3-iodophenyl)-6-((2-methylbut-3-yn-2-yl)oxy)hexan-2-one (Intermediate 2-23, 0.8 g, 1.73 mmol), the reaction procedure sequence (Steps A to E) described for Example 2 was used to prepare the title compound (112 mg) as a white solid. MS (ESI): 653 m/z [M+H]⁺, retention time: 1.69 minutes, purity: >99% (214 nm) (LC-MS method 2). ¹H NMR (400 MHz, CD₃OD) δ 7.57 (s, 1H), 7.44-7.20 (m, 5H), 7.17-7.12 (m, 2H), 7.08-6.99 (m, 2H), 6.80 (s, 1H), 6.16 (d, J=2.8 Hz, 1H), 5.99-5.89 (m, 2H), 3.79 (dd, J=10.0 Hz & 4.8 Hz, 1H), 3.27-3.20 (m, 1H), 3.16-3.02 (m, 1H), 2.85 (t, J=8.0 Hz, 2H), 2.48 (t, J=8.0 Hz, 2H), 2.31-2.20 (m, 1H), 1.82-1.71 (m, 1H), 1.55-1.37 (m, 8H) ppm.

Example 41. 41. 3-[3-(24,30-Difluoro-6,10,10-trimethyl-8,26-dioxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

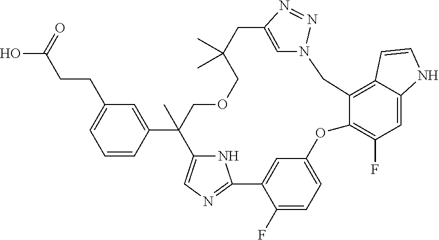 [SMILES: CC1(C)COCC(C)(c2cccc(CCC(=O)O)c2)c2cnc([nH]2)-c2cc(ccc2F)Oc2c(F)cc3[nH]ccc3c2Cn2cc(nn2)C1]

Exchanging 1-bromo-3-(3-iodophenyl)-3-methylnon-8-yn-2-one (Intermediate 2-51) with 1-bromo-4-((2,2-dimethylpent-4-yn-1-yl)oxy)-3-(3-iodophenyl)-3-methylbutan-2-one (Intermediate 33, 0.8 g, 1.73 mmol), the reaction procedure sequence (Steps A to E) described for Example 2 was used to prepare the title compound (32 mg) as a white solid. MS (ESI): 667 m/z [M+H]⁺, retention time: 1.76 minutes, purity: 98% (214 nm) (LC-MS method 2). ¹H NMR (400 MHz, CD₃OD) δ 7.43 (d, J=10.3 Hz, 1H), 7.37 (d, J=4.3 Hz, 1H), 7.29-7.22 (m, 3H), 7.17-7.11 (m, 2H), 7.08 (s, 1H), 7.05-7.01 (m, 2H), 6.89 (s, 1H), 5.89-5.80 (m, 1H), 5.75-5.65 (m, 1H), 3.76 (d, J=8.1 Hz, 1H), 3.55 (d, J=8.2 Hz, 1H), 2.85 (t, J=7.8 Hz, 2H), 2.74-2.68 (m, 1H), 2.64-2.58 (m, 1H), 2.53 (t, J=7.7 Hz, 2H), 2.39-2.34 (m, 1H), 2.27-2.21 (m, 1H), 1.61 (s, 3H), 0.85 (s, 3H), 0.70 (s, 3H) ppm

Example 42. Compound 42. 3-[3-(24,30-Difluoro-6-methyl-26-oxa-13-thia-3,21,32,33-tetrazahexacyclo[25.3.1.12,5.112,15.017,25.018,22]tritriaconta-1(31),2,4,12(32),14,17,19,22,24,27,29-undecaen-6-yl)phenyl]propanoic acid

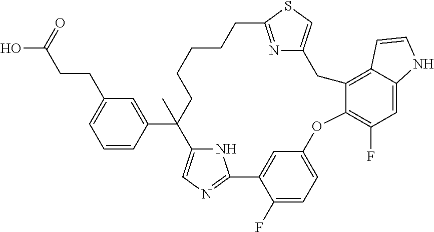 [SMILES: CC1(c2cccc(CCC(=O)O)c2)CCCCCc2nc(cs2)Cc2c(c(F)cc3[nH]ccc23)Oc2ccc(F)c(c2)-c2ncc1[nH]2]

Methyl (E)-3-(3-(2-(2-(2-fluoro-5-((6-fluoro-4-((2-iodothiazol-4-yl)methyl)-1-tosyl-1H-indol-5-yl)oxy)phenyl)-1H-imidazol-5-yl)hept-6-yn-2-yl)phenyl)acrylate

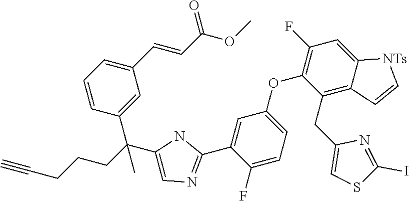 [SMILES: C#CCCCC(C)(c1cccc(/C=C/C(=O)OC)c1)c1cnc(-c2cc(Oc3c(F)cc4c(ccn4S(=O)(=O)c4ccc(C)cc4)c3Cc3csc(I)n3)ccc2F)[nH]1]

Step A: To a stirred solution of 2-fluoro-5-((6-fluoro-4-((2-iodothiazol-4-yl)methyl)-1-tosyl-1H-indol-5-yl)oxy)benzimidamide (Intermediate 30-1, 0.354 g, 0. 553 mmol) in dimethylformamide (5 mL) was added methyl (E)-3-(3-(1-bromo-3-methyl-2-oxooct-7-yn-3-yl)phenyl)acrylate (Intermediate 2-21, 0.201 g, 0. 553 mmol) and sodium bicarbonate (0.0895 g, 1.07 mmol). The reaction was stirred at 75° C. for 8 hours and quenched with water (15 mL). The precipitate was collected and purified by automated silica gel column chromatography (12 g column, eluting with petroleum ether ethyl acetate=4:1 to 1:1) to give the title compound (311 mg, 63%) as an off-white solid. MS (ESI): 943 m/z [M+H]⁺, retention time: 2.02 minutes, purity: 95% (254 nm) (LC-MS method 6).

Methyl (E)-3-[3-[24,30-difluoro-6-methyl-21-(p-tolylsulfonyl)-26-oxa-13-thia-3,21,32,33-tetrazahexacyclo[25.3.1.12,5.112,15.017,25.018,22]tritriaconta-1(31),2,4,12(32),14,17,19,22,24,27,29-undecaen-10-yn-6-yl]phenyl]prop-2-enoate

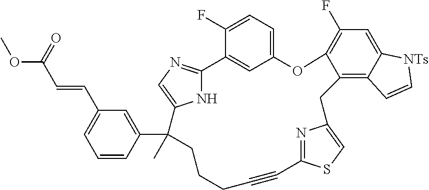 [SMILES: COC(=O)/C=C/c1cccc(C2(C)CCCC#Cc3nc(cs3)Cc3c(c(F)cc4c3ccn4S(=O)(=O)c3ccc(C)cc3)Oc3ccc(F)c(c3)-c3ncc2[nH]3)c1]

Step B: To a stirred solution of Step A product (100 mg, 0.106 mmol) in N,N-dimethylformamide (10 mL) was added bis(triphenylphosphine) palladium (II) dichloride (7.5 mg, 0.011 mmol), copper (I) iodide (4 mg, 0.022 mmol) and triethylamine (32 mg, 0.318 mmol). The reaction was stirred at 60° C. for 16 hours and quenched with water (40 mL), then the mixture was extracted with ethyl acetate (3×50 mL). The combined organic phases were dried over sodium sulfate and concentrated. The residue was purified by automated silica gel column chromatography (12 g column, eluting with petroleum ether: ethyl acetate=4:1 to 1:1) to give the title compound (13 mg, 15.0%) as a yellow solid. MS (ESI): 815 m/z [M+H]⁺, retention time: 1.99 minutes, purity: 90% (254 nm) (LC-MS method 2).

Methyl 3-[3-[24,30-difluoro-6-methyl-21-(p-tolylsulfonyl)-26-oxa-13-thia-3,21,32,33-tetrazahexacyclo[25.3.1.12,5.112,15.017,25.018,22]tritriaconta-1(31),2,4,12(32),14,17,19,22,24,27,29-undecaen-6-yl]phenyl]propanoate

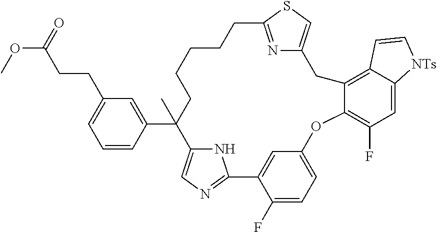 [SMILES: COC(=O)CCc1cccc(C2(C)CCCCCc3nc(cs3)Cc3c(c(F)cc4c3ccn4S(=O)(=O)c3ccc(C)cc3)Oc3ccc(F)c(c3)-c3ncc2[nH]3)c1]

Step C: To a stirred solution of Step B product (0.010 g, 0.0123 mmol) in methanol (4 mL) was added Raney Nickel (0.010 g, 0.17 mmol). The mixture was stirred under hydrogen balloon at room temperature for 12 hours, then filtered through a pad of Celite. The filtrate was concentrated to give the title compound (5 mg, 50%) as a grey solid. MS (ESI): 821 m/z [M+H]⁺, retention time: 2.10 minutes, purity: 83% (254 nm) (LC-MS method 2).

Compound 42. 3-[3-(24,30-Difluoro-6-methyl-26-oxa-13-thia-3,21,32,33-tetrazahexacyclo[25.3.1.12,5.112,15.017,25.018,22]tritriaconta-1(31),2,4,12(32),14,17,19,22,24,27,29-undecaen-6-yl)phenyl]propanoic acid

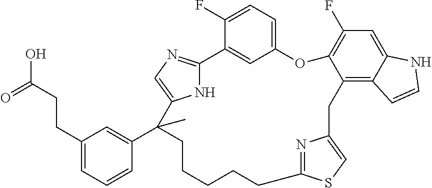 [SMILES: CC1(c2cccc(CCC(=O)O)c2)CCCCCc2nc(cs2)Cc2c(c(F)cc3[nH]ccc23)Oc2ccc(F)c(c2)-c2ncc1[nH]2]

Step D: To a stirred solution of Step C product (20 mg, 0.0244 mmol) in methanol (4 mL) was added lithium hydroxide monohydrate (10.2 mg, 0.244 mmol) at 80° C. for 3 hours, and then concentrated under reduced pressure. The residue was purified by preparative HPLC to give the title compound (2.20 mg, 14%) as white solid. MS (ESI): 653 m/z [M+H]⁺, retention time: 1.83 minutes, purity: 97% (254 nm) (LC-MS method 9). ¹H NMR (400 MHz, CD₃OD) δ 7.30-7.29 (m, 1H), 7.25-7.15 (m, 3H), 7.11-7.02 (m, 5H), 6.88-6.86 (m, 1H), 6.63 (s, 1H), 6.60-6.58 (m, 1H), 4.28-4.26 (m, 2H), 2.85 (t, J=7.8 Hz, 2H), 2.58-2.56 (m, 2H), 2.51 (t, J=7.8 Hz, 2H), 2.28-2.20 (m, 1H), 2.18-2.02 (m, 1H), 1.66-1.53 (m, 7H), 1.40-1.31 (m, 2H) ppm

Example 43. Compound 43. 3-[3-(24,30-difluoro-9,9-dimethyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)-2-fluoro-phenyl]propanoic acid

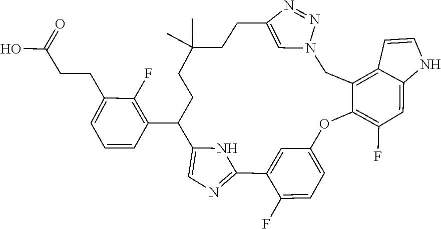 [SMILES: CC1(C)CCc2cn(nn2)Cc2c(c(F)cc3[nH]ccc23)Oc2ccc(F)c(c2)-c2ncc([nH]2)C(c2cccc(CCC(=O)O)c2F)CC1]

Exchanging 1-bromo-3-(3-iodophenyl)-3-methylnon-8-yn-2-one (Intermediate 2-51) with 1-bromo-3-(3-bromo-2-fluorophenyl)-6,6-dimethyldec-9-yn-2-one (Intermediate 2-25, 432 mg, 1.0 mmol), the reaction procedure sequence (Steps A to E) described for Example 2 was used to prepare the title compound (9.2 mg) as a white solid. MS (ESI): 669 m/z [M+H]⁺, retention time: 1.78 minutes, purity: 92% (214 nm) (LC-MS method 5). ¹H NMR (400 MHz, DMSO-d₆) δ 11.87 (s, 1H), 11.58 (s, 1H), 7.53-7.46 (m, 2H), 7.32-7.03 (m, 6H), 6.93-6.83 (m, 3H), 6.55 (s, 1H), 5.84 (s, 2H), 4.03-3.97 (m, 1H), 2.79 (t, J=7.4 Hz, 2H), 2.46 (t, J=7.6 Hz, 2H), 2.27-2.33 (m, 1H), 2.16-2.22 (m, 1H), 1.96-2.04 (m, 2H), 1.30 (s, 2H), 1.15-1.09 (m, 2H), 0.77 (s, 6H) ppm.

Example 44. Compound 44. 3-[3-(24,30-Difluoro-6-methyl-26-oxa-3,14,15,21,33-pentazahexacyclo[25.3.1.12,5.112,15.017,25.018,22]tritriaconta-1(31),2,4,12(32),13,17,19,22,24,27,29-undecaen-6-yl)phenyl]propanoic acid

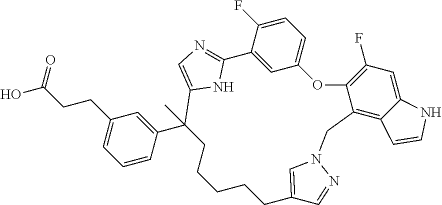 [SMILES: CC1(c2cccc(CCC(=O)O)c2)CCCCCc2cnn(c2)Cc2c(c(F)cc3[nH]ccc23)Oc2ccc(F)c(c2)-c2ncc1[nH]2]

Exchanging 5-((4-((4-bromothiazol-2-yl)methyl)-6-fluoro-1H-indol-5-yl)oxy)-2-fluorobenzimidamide (Intermediate 30) with 5-((4-((4-bromo-1H-pyrazol-1-yl)methyl)-6-fluoro-1H-indol-5-yl)oxy)-2-fluorobenzimidamide (Intermediate 6-7, 290 mg, 0.76 mmol) and methyl (E)-3-(3-(1-bromo-3-methyl-2-oxooct-7-yn-3-yl)phenyl)acrylate (Intermediate 2-20) with methyl (E)-3-(3-(1-bromo-3-methyl-2-oxooct-7-yn-3-yl)phenyl)acrylate (Intermediate 2-21, 377 mg, 0.84 mmol), the reaction procedure sequence (Steps A to E) described for Example 36 was used to prepare the title compound (13 mg) as a white solid. MS (ESI): 636 m/z [M+H]⁺, retention time: 1.46 minutes, purity: >99% (254 nm) (LC-MS method 5). ¹H NMR (400 MHz, CD₃OD) 7.27-7.35 (m, 5H), 7.16-7.20 (m, 1H), 7.12 (s, 1H), 7.01-7.06 (m, 4H), 6.93 (s, 1H), 6.52-6.53 (m, 1H), 5.47-5.59 (m, 2H), 2.84-2.87 (m, 2H), 2.52-2.56 (m, 2H), 2.02-2.131 (m, 4H), 1.54 (s, 3H), 1.27-1.30 (m, 2H), 1.18-1.22 (m, 2H), 0.90-0.92 (m, 2H) ppm.

Example 45. Compound 45A, 45B. Diastereomers 1 and 2 of 3-[3-(24,30-difluoro-9-methyl-26-oxa-3,13,14,15,21,33-hexazahexacyclo[25.3.1.12,5.112,15.017,25.018,22]-tritriaconta-1(31),2,4,12(32),13,17,19,22,24,27,29-undecaen-6-yl)-2-fluoro-phenyl]propanoic acid (relative stereochemistry unknown)

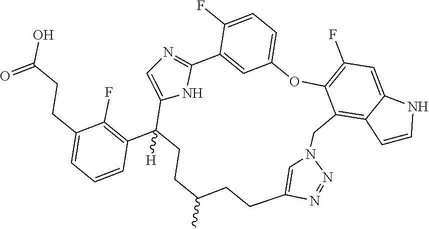 [SMILES: CC1CCc2cn(nn2)Cc2c(c(F)cc3[nH]ccc23)Oc2ccc(F)c(c2)-c2ncc([nH]2)C(c2cccc(CCC(=O)O)c2F)CC1]

Exchanging 1-bromo-3-(3-iodophenyl)-3-methylnon-8-yn-2-one (Intermediate 2-51) with 1-bromo-3-(3-bromo-2-fluorophenyl)-6-methyldec-9-yn-2-one (Intermediate 2-26, 710 mg, 1.7 mmol), the reaction procedure sequence (Steps A to E) described for Example 2 was used to prepare the title compounds. The stereo-isomeric acids, due to two chiral centers, were separated by prep-thin layer chromatography at corresponding Step E of Example 2. The first band (5.2 mg) was designated as Compound 45A; The second band (13.8 mg), was designated as Compound 45B.

Compound 45A: MS (ESI): 655 m/z [M+H]⁺, retention time: 1.71 minutes, purity: >99% (254 nm) (LC-MS method 2). ¹H NMR (400 MHz, CD₃OD) δ 7.38 (d, J=10.7 Hz, 1H), 7.33 (d, J=3.0 Hz, 2H), 7.19 (m, 3H), 7.11-7.01 (m, 2H), 6.93-6.84 (m, 2H), 6.43 (d, J=3.1 Hz, 1H), 5.84 (s, 2H), 4.13 (d, J=9.2 Hz, 1H), 2.89 (m, 2H), 2.47 (m, 2H), 2.29 (m, 2H), 2.00 (m, 2H), 1.72 (m, 1H), 1.59 (m, 1H), 1.21-1.05 (m, 3H), 0.81 (d, J=6.6 Hz, 3H) ppm.

Compound 45B: MS (ESI): 655 m/z [M+H]⁺, retention time: 1.68 minutes, purity: >99% (254 nm) (LC-MS method 2). ¹H NMR (400 MHz, CD₃OD) δ 7.33-7.25 (m, 3H), 7.07 (m, 3H), 6.97 (t, J=6.8 Hz, 1H), 6.89-6.78 (m, 3H), 6.48 (d, J=3.1 Hz, 1H), 5.81-5.70 (m, 2H), 4.08 (dd, J=10.5, 3.4 Hz, 1H), 2.79 (m, 2H), 2.43 (m, 2H), 2.21 (m, 1H), 2.13-1.99 (m, 1H), 1.93 (m, 1H), 1.68 (m, 1H), 1.48-1.22 (m, 5H), 0.76 (d, J=6.4 Hz, 3H) ppm.

Example 46. Compound 46. 3-[3-(24,30-Difluoro-6,11-dimethyl-26-oxa-3,13,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]propanoic acid

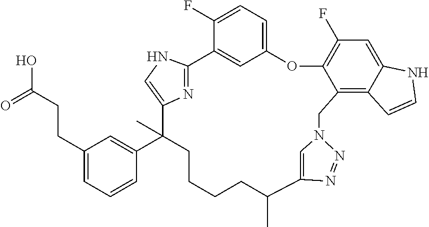 [SMILES: CC1CCCCC(C)(c2cccc(CCC(=O)O)c2)c2c[nH]c(n2)-c2cc(ccc2F)Oc2c(F)cc3[nH]ccc3c2Cn2cc1nn2]

Exchanging 1-bromo-3-(3-iodophenyl)-3-methylnon-8-yn-2-one (Intermediate 2-51) with 1-bromo-3-(3-iodophenyl)-3,8-dimethyldec-9-yn-2-one (Intermediate 2-27, 512 mg, 1.11 mmol), the reaction procedure sequence (Steps A to E) described for Example 2 was used to prepare the title compound (130.9 mg) as a mixture of inseparable diastereomers (originated from two chiral centers). MS (ESI): 651 m/z [M+H]⁺, retention time: 1.75 minutes, purity: >99% (254 nm) (LC-MS method 2).

Example 47. Compound 47. 3-[3-(25,31-Difluoro-6-methyl-27-oxa-3,14,15,16,22,34-hexazahexacyclo[26.3.1.12,5.113,16.018,26.019,23]tetratriaconta-1(32),2,4,13(33),14,18,20,23,25,28,30-undecaen-6-yl)phenyl]propanoic acid

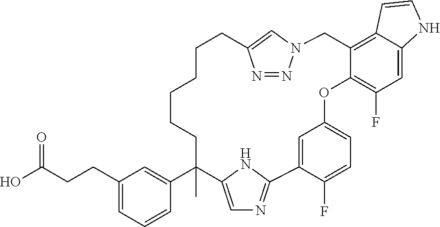 [SMILES: CC1(c2cccc(CCC(=O)O)c2)CCCCCCc2cn(nn2)Cc2c(c(F)cc3[nH]ccc23)Oc2ccc(F)c(c2)-c2ncc1[nH]2]

Exchanging 1-bromo-3-(3-iodophenyl)-3-methylnon-8-yn-2-one (Intermediate 2-51) with 1-bromo-3-(3-iodophenyl)-3-methylundec-10-yn-2-one (Intermediate 2-28, 707 mg, 1.53 mmol), the reaction procedure sequence (Steps A to E) described for Example 2 was used to prepare the title compound (51 mg) as a white solid. MS (ESI): 651 m/z [M+H]⁺, retention time: 1.85 minutes, purity: >99% (214 nm) (LC-MS method 11). ¹H NMR (400 MHz, CD₃OD) δ 7.39-7.36 (m, 2H), 7.25-7.03 (m, 8H), 6.97 (s, 1H), 6.60 (d, J=3.1 Hz, 1H), 5.90-5.70 (m, 2H), 2.88 (t, J=7.7 Hz, 2H), 2.56 (t, J=7.7 Hz, 2H), 2.43-2.39 (m, 2H), 2.27-1.92 (m, 2H), 1.54 (s, 3H), 1.43-1.28 (m, 6H), 0.90-0.87 (m, 2H) ppm.

Example 48. Compound 48. 3-[3-(24,30-Difluoro-6,11,11-trimethyl-26-oxa-3,4,13,14,15,21,33-heptazahexacyclo[25.3.1.12,5.112,15.017,25.018,22]tritriaconta-1(31),2,4,12(32),13,17,19,22,24,27,29-undecaen-6-yl)phenyl]propanoic acid

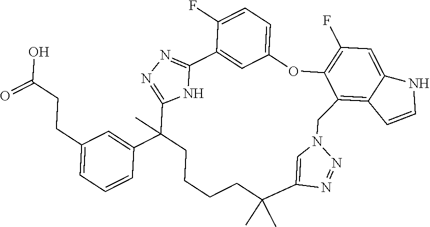 [SMILES: CC1(C)CCCCC(C)(c2cccc(CCC(=O)O)c2)c2nnc([nH]2)-c2cc(ccc2F)Oc2c(F)cc3[nH]ccc3c2Cn2cc1nn2]

(6-Fluoro-5-(4-fluoro-3-(5-(2-(3-iodophenyl)-7,7-dimethylnon-8-yn-2-yl)-4H-1,2,4-triazol-3-yl)phenoxy)-1H-indol-4-yl)methanol

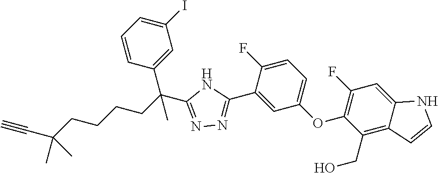 [SMILES: C#CC(C)(C)CCCCC(C)(c1cccc(I)c1)c1nnc(-c2cc(Oc3c(F)cc4[nH]ccc4c3CO)ccc2F)[nH]1]

Step A: To a solution of 2-fluoro-5-((6-fluoro-4-(hydroxymethyl)-1H-indol-5-yl)oxy)-benzimidamide (Intermediate 6-6, 1.1 g, 3.47 mmol) in methanol (20 mL) was added sodium methoxide (5.4 M in methanol, 0.95 mL, 5.21 mmol), 2-(3-iodophenyl)-2,7,7-trimethylnon-8-ynehydrazide (Intermediate 39, 2.15 g, 5.21 mmol). The mixture was stirred at 100° C. for 1 hour in a microwave. Another portion of sodium methoxide (5.4 M in methanol, 0.95 mL, 5.37 mmol) and 2-(3-iodophenyl)-2,7,7-trimethyl-non-8-ynehydrazide (Intermediate 39, 2.15 g, 5.37 mmol) was added into the reaction mixture and stirred at 100° C. for an additional 1 hour in the microwave. The mixture was diluted with ethyl acetate (40 mL), acidified to pH ˜5 using 1 M hydrochloric acid. The solution was washed with water, brine, dried over sodium sulfate, and concentrated. The residue was purified by automated flash chromatography (20 g silica gel column, eluting with 0-60% ethyl acetate in petroleum ether) to give the title compound (780 mg, 32%) as a solid. MS (ESI): 695 m/z [M+H]⁺, retention time: 2.24 minutes, purity: 90% (214 nm) (LC-MS method 2).

4-(Azidomethyl)-5-(3-(5-(7,7-dimethyl-2-phenylnon-8-yn-2-yl)-1H-1,2,4-triazol-3-yl)-4-fluorophenoxy)-6-fluoro-1-tosyl-1H-indole

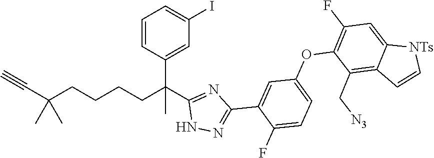 [SMILES: C#CC(C)(C)CCCCC(C)(c1cccc(I)c1)c1nc(-c2cc(Oc3c(F)cc4c(ccn4S(=O)(=O)c4ccc(C)cc4)c3CN=[N+]=[N-])ccc2F)n[nH]1]

Step B: To a stirred and cooled (0° C.) solution of Step A product (1 g, 1.44 mmol) in dichloromethane (20 mL) was added N—N-dimethylaminopyridine (17.6 mg, 0.14 mmol), triethylamine (642 uL, 4.61 mmol), and a solution of tosyl chloride (878 mg, 4.61 mmol) in dichloromethane (2 mL). The reaction was stirred at room temperature overnight and diluted with dichloromethane (20 mL). The solution was washed with water, brine, dried over sodium sulfate, and concentrated. To a stirred solution of the residue in N,N-dimethylformamide was added sodium azide (281 mg, 4.32 mmol). The solution was stirred at 65° C. overnight, cooled to room temperature, and diluted with ethyl acetate (30 mL). The mixture was washed with water, brine, dried over sodium sulfate, and concentrated. The residue was purified by automated flash chromatography (40 g silica gel column, eluting with 0-75% ethyl acetate in petroleum ether) to give the title compound (0.38 g, 30%) as a solid. MS (ESI): 874 m/z [M+H]⁺, retention time: 2.53 minutes, purity: 71% (214 nm) (LC-MS method 2).

4-(Azidomethyl)-6-fluoro-5-(4-fluoro-3-(5-(2-(3-iodophenyl)-7,7-dimethylnon-8-yn-2-yl)-4H-1,2,4-triazol-3-yl)phenoxy)-1H-indole

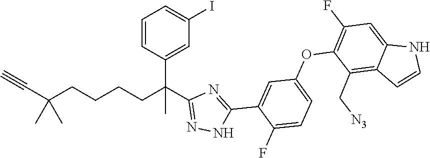 [SMILES: C#CC(C)(C)CCCCC(C)(c1cccc(I)c1)c1n[nH]c(-c2cc(Oc3c(F)cc4[nH]ccc4c3CN=[N+]=[N-])ccc2F)n1]

Step C: To a stirred solution of Step B product (0.38 g, 0.44 mmol) in tetrahydrofuran/water/methanol (12 mL/3 mL/3 mL) was added lithium hydroxide monohydrate (91 mg, 2.17 mmol). The reaction was stirred at room temperature for 2 hours. The mixture was acidified to pH ˜4 with 1 M hydrochloric acid and diluted with ethyl acetate (30 mL), washed with brine (10 mL), dried over sodium sulfate, and concentrated. The residue was purified by automated flash chromatography (20 g silica gel column, eluting with 0-75% ethyl acetate in petroleum ether) to give the title compound (0.24 g, 77%) as a solid. MS (ESI): 720 m/z [M+H]⁺, retention time: 2.42 minutes, purity: 81% (214 nm) (LC-MS method 9).

Compound 48: 3-[3-(24,30-Difluoro-6,11,11-trimethyl-26-oxa-3,4,13,14,15,21,33-heptazahexacyclo[25.3.1.12,5.112,15.017,25.018,22]tritriaconta-1(31),2,4,12(32),13,17,19,22,24,27,29-undecaen-6-yl)phenyl]propanoic acid

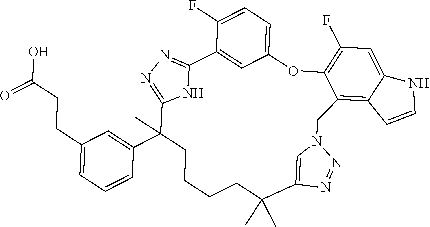 [SMILES: CC1(C)CCCCC(C)(c2cccc(CCC(=O)O)c2)c2nnc([nH]2)-c2cc(ccc2F)Oc2c(F)cc3[nH]ccc3c2Cn2cc1nn2]

Exchanging 4-(azidomethyl)-6-fluoro-5-(4-fluoro-3-(5-(2-(3-iodophenyl)oct-7-yn-2-yl)-1H-imidazol-2-yl)phenoxy)-1H-indole (Step A product of Example 2) with 4-(azidomethyl)-6-fluoro-5-(4-fluoro-3-(5-(2-(3-iodophenyl)-7,7-dimethylnon-8-yn-2-yl)-4H-1,2,4-triazol-3-yl)phenoxy)-1H-indole (Step C product of this example, 250 mg, 0.35 mmol) the reaction procedure sequence (Steps B to E) described for Example 2 was used to prepare the title compound (60 mg) as a white solid. MS (ESI): 666 m/z [M+H]⁺, retention time: 1.99 minutes, purity: 97% (214 nm) (LC-MS method 2). ¹H NMR (400 MHz, CD₃OD) δ 7.38-7.31 (m, 5H), 7.14 (t, J=7.1 Hz, 2H), 7.07-6.99 (m, 2H), 6.94 (d, J=7.9 Hz, 1H), 6.68 (s, 1H), 6.00 (d, J=14.5 Hz, 1H), 5.78 (d, J=14.1 Hz, 1H), 2.84 (t, J=7.7 Hz, 2H), 2.52 (t, J=7.7 Hz, 2H), 2.10-1.96 (m, 2H), 1.63 (s, 3H), 1.51-1.42 (m, 2H), 1.34-1.27 (m, 1H), 1.16 (s, 3H), 1.08 (s, 3H), 1.04-1.0 (m, 1H), 0.78-0.61 (m, 2H) ppm.

Example 49. Compound 49. 3-[3-(24,30-Difluoro-6,14-dimethyl-26-oxa-3,14,21,32,33-pentazahexacyclo[25.3.1.12,5.112,15.017,25.018,22]tritriaconta-1(30),2,4,12,15(32),17(25),18(22),19,23,27(31),28-undecaen-6-yl)phenyl]propanoic acid

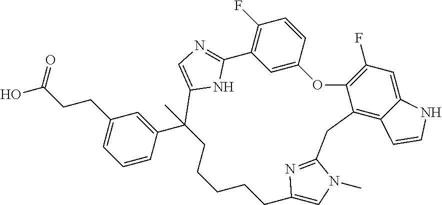 [SMILES: Cn1cc2nc1Cc1c(c(F)cc3[nH]ccc13)Oc1ccc(F)c(c1)-c1ncc([nH]1)C(C)(c1cccc(CCC(=O)O)c1)CCCCC2]

Ethyl 2-(6-fluoro-5-(4-fluoro-3-(5-(2-(3-iodophenyl)-7-(3-methyl-2-oxooxazolidin-5-yl)heptan-2-yl)-1H-imidazol-2-yl)phenoxy-1H-indol-4-yl)acetate

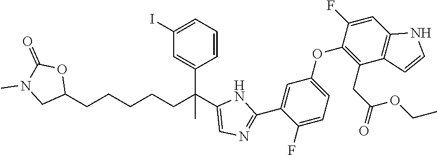 [SMILES: CCOC(=O)Cc1c(Oc2ccc(F)c(-c3ncc(C(C)(CCCCCC4CN(C)C(=O)O4)c4cccc(I)c4)[nH]3)c2)c(F)cc2[nH]ccc12]

Step A: In glove box, to a stirred solution of 5-(8-chloro-6-(3-iodophenyl)-6-methyl-7-oxooctyl)-3-methyloxazolidin-2-one (Intermediate 2-29, 639 mg, 1.34 mmol) in dry dimethylformamide (6.5 mL) was added ethyl 2-(5-(3-carbamimidoyl-4-fluorophenoxy)-6-fluoro-1H-indol-4-yl)acetate (Intermediate 29, 500 mg, 1.34 mmol) and sodium bicarbonate (225 mg, 2.68 mmol). The reaction was stirred at 80° C. overnight, cooled to room temperature and quenched with water. The solution was extracted with ethyl acetate (3×20 mL). The combined organic extracts were washed with brine, dried over sodium sulfate, and concentrated. The residue was purified by silica gel column chromatography (20 g silica gel column, eluting with 0-50% ethyl acetate in petroleum ether) to give the title compound (793 mg, 74%) as a yellow solid. MS (ESI): 797 m/z [M+H]⁺, retention time: 1.84 minutes, purity: >99% (214 nm) (LC-MS method 2).

2-(6-Fluoro-5-(4-fluoro-3-(5-(8-hydroxy-2-(3-iodophenyl)-9-(methylamino)nonan-2-yl)-1H-imidazol-2-yl)phenoxy)-1H-indol-4-yl)acetic acid

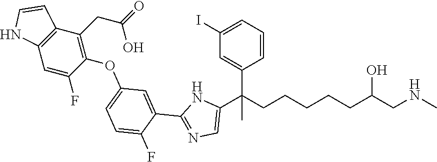 [SMILES: CNCC(O)CCCCCC(C)(c1cccc(I)c1)c1cnc(-c2cc(Oc3c(F)cc4[nH]ccc4c3CC(=O)O)ccc2F)[nH]1]

Step B: To a solution of Step A product (793 mg, 0.996 mmol) in tetrahydrofuran (3.6 mL), water (3.6 mL) and methanol (3.6 mL) was added lithium hydroxide (836 mg, 19.92 mmol), the mixture was stirred at 60° C. overnight, cooled to room temperature and adjusted to pH ˜6 using 1 M hydrochloric acid. The solution was extracted with dichloromethane (contains 5% methanol, 5×50 mL). The combined organic extracts were dried over sodium sulfate and concentrated to give the title compounds (754 mg, 90%) as a yellow solid. MS (ESI): 743 m/z [M+H]⁺, retention time: 1.78 minutes, purity: >99% (254 nm) (LC-MS method 9).

Compound 49: 3-[3-(24,30-Difluoro-6,14-dimethyl-26-oxa-3,14,21,32,33-pentazahexacyclo[25.3.1.12,5.112,15.017,25.018,22]tritriaconta-1(30),2,4,12,15(32),17(25),18(22),19,23,27(31),28-undecaen-6-yl)phenyl]propanoic acid

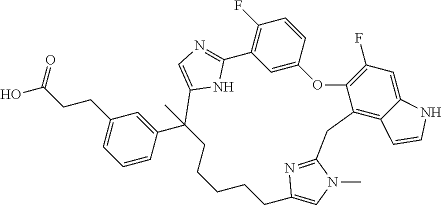 [SMILES: Cn1cc2nc1Cc1c(c(F)cc3[nH]ccc13)Oc1ccc(F)c(c1)-c1ncc([nH]1)C(C)(c1cccc(CCC(=O)O)c1)CCCCC2]

Step C: Exchanging (E)-3-(5-(3-(5-(7-amino-6-hydroxy-2-(3-iodophenyl)heptan-2-yl)-1H-imidazol-2-yl)-4-fluorophenoxy)-6-fluoro-1H-indol-4-yl)acrylic acid (Step C product of Example 21) with 2-(6-Fluoro-5-(4-fluoro-3-(5-(8-hydroxy-2-(3-iodophenyl)-9-(methylamino)-nonan-2-yl)-1H-imidazol-2-yl)phenoxy)-1H-indol-4-yl)acetic acid (Step B product of this Example, 754 mg, 1.01 mmol) the reaction procedure sequence (Steps D to G) described for Example 21, followed by the reaction sequence (Steps A to B) of Example 22 were used to prepare the title compound (5.2 mg) as a white solid. MS (ESI): 650 m/z [M+H]⁺, retention time: 1.75 minutes, purity: >99% (254 nm) (LC-MS method 13).

Example 50. Compound 50. 3-[3-(24,30-Difluoro-6,11,11-trimethyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

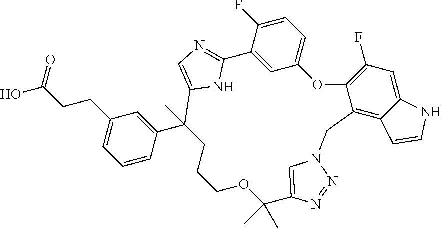 [SMILES: CC1(C)OCCCC(C)(c2cccc(CCC(=O)O)c2)c2cnc([nH]2)-c2cc(ccc2F)Oc2c(F)cc3[nH]ccc3c2Cn2cc1nn2]

Exchanging 1-bromo-3-(3-iodophenyl)-3-methylnon-8-yn-2-one (Intermediate 2-51) with 3-(3-bromophenyl)-1-chloro-3-methyl-6-((2-methylbut-3-yn-2-yl)oxy)hexan-2-one (Intermediate 2-30, 460 mg, 1.2 mmol), the reaction procedure sequence (Steps A to E) described for Example 2 was used to prepare the title compound (62 mg) as a white solid. MS (ESI): 667 m/z [M+H]⁺, retention time: 1.39 minutes, purity: >99% (214 nm) (LC-MS method 13). ¹H NMR (400 MHz, CD₃OD) δ 7.47 (s, 1H), 7.33-7.26 (m, 4H), 7.21 (d, J=4.4 Hz, 1H), 7.13-6.93 (m, 5H), 6.36 (d, J=2.8 Hz, 1H), 5.92 (d, J=14.8 Hz, 1H), 5.84 (d, J=14.8 Hz, 1H), 3.20-3.12 (m, 1H), 2.89 (q, J=7.6 Hz, 1H), 2.81 (t, J=7.6 Hz, 2H), 2.47 (t, J=7.6 Hz, 2H), 2.23-2.16 (m, 1H), 1.99-1.88 (m, 1H), 1.57-1.45 (m, 4H), 1.43 (s, 3H), 1.34 (s, 3H), 1.21-1.08 (m, 1H) ppm.

Example 51. Compound 51. 3-[3-(24,30-Difluoro-6,10,10-trimethyl-26-oxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

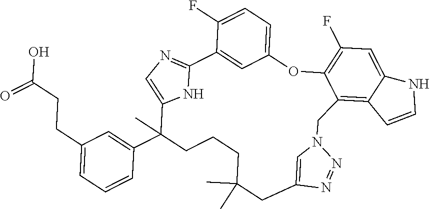 [SMILES: CC1(C)CCCC(C)(c2cccc(CCC(=O)O)c2)c2cnc([nH]2)-c2cc(ccc2F)Oc2c(F)cc3[nH]ccc3c2Cn2cc(nn2)C1]

Exchanging 1-bromo-3-(3-iodophenyl)-3-methylnon-8-yn-2-one (Intermediate 2-51) with 1-bromo-3-(3-iodophenyl)-3,7,7-trimethyldec-9-yn-2-one (Intermediate 2-31, 0.764 g, 1.61 mmol), the reaction procedure sequence (Steps A to E) described for Example 2 was used to prepare the title compound (178 mg) as a white solid. MS (ESI): 665 m/z [M+H]⁺, retention time: 2.39 minutes, purity: >99% (214 nm) (LC-MS method 11). ¹H NMR (400 MHz, CD₃OD) δ 7.39-7.25 (m, 5H), 7.16-7.08 (m, 4H), 7.00-6.96 (m, 2H), 5.98 (s, 1H), 5.93-5.70 (m, 2H), 2.84 (t, J=7.6 Hz, 2H), 2.53 (t, J=7.6 Hz, 2H), 2.38 (m, 2H), 1.89-1.84 (m, 2H), 1.55 (s, 3H), 1.32-1.24 (m, 1H), 0.89-0.82 (m, 5H), 0.76-0.70 (m, 4H) ppm.

Example 52. Compound 52. 3-[3-(24,30-Difluoro-6-methyl-26,32-dioxa-3,13,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

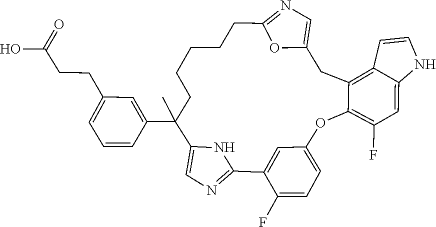 [SMILES: CC1(c2cccc(CCC(=O)O)c2)CCCCCC2=NC=C(Cc3c(c(F)cc4[nH]ccc34)Oc3ccc(F)c(c3)-c3ncc1[nH]3)O2]

7-(2-(2-fluoro-5-((6-fluoro-4-((2-oxooxazolidin-5-yl)methyl)-1H-indol-5-yl)oxy)phenyl)-1H-imidazol-5-yl)-7-(3-iodophenyl)octanenitrile

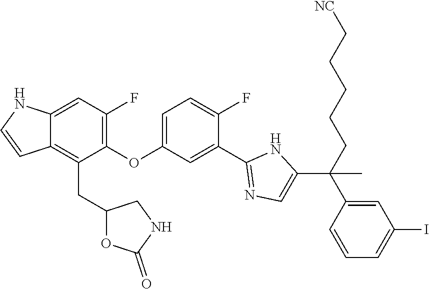 [SMILES: CC(CCCCCC#N)(c1cccc(I)c1)c1cnc(-c2cc(Oc3c(F)cc4[nH]ccc4c3CC3CNC(=O)O3)ccc2F)[nH]1]

Step A: To a stirred solution of 9-bromo-7-(3-iodophenyl)-7-methyl-8-oxononanenitrile (Intermediate 2-32, 1.459 g, 3.780 mmol) in N,N-dimethylformamide (15 mL) was added sodium bicarbonate (0.529 g, 6.3 mmol) and 2-fluoro-5-((6-fluoro-4-((2-oxooxazolidin-5-yl)methyl)-1H-indol-5-yl)oxy)benzimidamide (Intermediate 6-8, 1.408 g, 3.150 mmol). The mixture was stirred at 70° C. overnight, cooled to room temperature, and quenched with water (50 mL). The brown mixture was extracted with ethyl acetate (3×40 mL), the combined organic layers were washed with aqueous lithium chloride, brine, dried over sodium sulfate, and concentrated. The residue was purified by automated flash chromatography (80 g silica gel column, eluting with 7% methanol in dichloromethane) to give the title compound (1.93 g, 66.8%) as yellow oil. MS (ESI): 736 m/z [M+H]⁺, retention time: 1.78 minutes, purity: 80% (214 nm) (LC-MS method 14).

7-(2-(5-((4-(3-Amino-2-hydroxypropyl)-6-fluoro-1H-indol-5-yl)oxy)-2-fluorophenyl)-1H-imidazol-5-yl)-7-(3-iodophenyl)octanoic acid

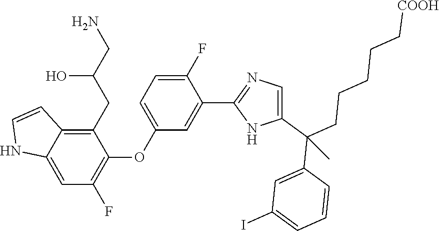 [SMILES: CC(CCCCCC(=O)O)(c1cccc(I)c1)c1cnc(-c2cc(Oc3c(F)cc4[nH]ccc4c3CC(O)CN)ccc2F)[nH]1]

Step B: To a stirred solution of Step A product (0. 1 g, 0.0136 mmol) in ethanol (1 mL)/water (0.2 mL) was added potassium hydroxide (0.00763 g, 0.136 mmol). The yellow mixture was stirred at 80° C. overnight, cooled to room temperature and diluted with water (20 mL). The aqueous mixture was washed with ethyl acetate (10 mL), acidified to pH ˜6, and extracted with dichloromethane/isopropanol (3/1) (3×20 mL). The combined organic phases were then dried over sodium sulfate and concentrated to afford the crude title compound (0.064 g, 64%) as a pale-yellow solid. MS (ESI): 729 m/z [M+H]⁺, retention time: 1.49 minutes, purity: 97% (214 nm) (LC-MS method 14).

Compound 52: 3-[3-(24,30-Difluoro-6-methyl-26,32-dioxa-3,13,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

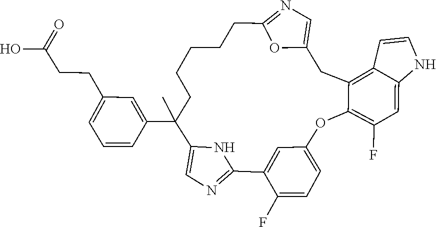 [SMILES: CC1(c2cccc(CCC(=O)O)c2)CCCCCC2=NC=C(Cc3c(c(F)cc4[nH]ccc34)Oc3ccc(F)c(c3)-c3ncc1[nH]3)O2]

Step C: Exchanging (14E)-23,29-difluoro-10-hydroxy-6-(3-iodophenyl)-6-methyl-25-oxa-3,12,20,31-tetrazapentacyclo[24.3.1.12,5.016,24.017,21]hentriaconta-1(30),2,4,14,16,18,21,23,26,28-decaen-13-one (Step C product of Example 21) with 7-(2-(5-((4-(3-Amino-2-hydroxypropyl)-6-fluoro-1H-indol-5-yl)oxy)-2-fluorophenyl)-1H-imidazol-5-yl)-7-(3-iodo-phenyl)octanoic acid (Step B product of this example, 0.064 g, 0.09 mmol), the reaction procedure sequence (Steps D to I) described for Example 21 was used to prepare the title compound (2 mg). MS (ESI): 637 m/z [M+H]⁺, retention time: 1.42 minutes, purity: 97% (214 nm) (LC-MS method 14).

Example 53. Compound 53. 3-[3-(24,30-Difluoro-6-methyl-26-oxa-3,13,15,21,33-pentazahexacyclo[25.3.1.12,5.112,15.017,25.018,22]tritriaconta-1(31),2,4,12(32),13,17,19,22,24,27,29-undecaen-6-yl)phenyl]propanoic acid

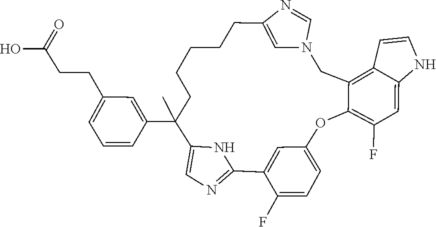 [SMILES: CC1(c2cccc(CCC(=O)O)c2)CCCCCc2cn(cn2)Cc2c(c(F)cc3[nH]ccc23)Oc2ccc(F)c(c2)-c2ncc1[nH]2]

Ethyl (E)-3-(3-(2-(2-(2-fluoro-5-((6-fluoro-4-((4-iodo-1H-imidazol-1-yl)methyl)-1H-indol-5-yl)oxy)phenyl)-1H-imidazol-5-yl)hept-6-yn-2-yl)phenyl)acrylate

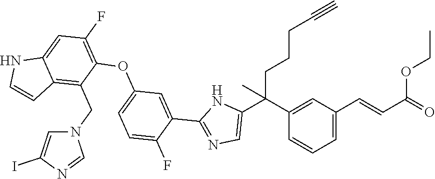 [SMILES: C#CCCCC(C)(c1cccc(/C=C/C(=O)OCC)c1)c1cnc(-c2cc(Oc3c(F)cc4[nH]ccc4c3Cn3cnc(I)c3)ccc2F)[nH]1]

Step A: To a stirred solution of 2-fluoro-5-((6-fluoro-4-((4-iodo-1H-imidazol-1-yl)methyl)-1H-indol-5-yl)oxy)benzimidamide (0.7 g, 0.92 mmol) and ethyl (E)-3-(3-(1-bromo-3-methyl-2-oxooct-7-yn-3-yl)phenyl)acrylate (0.7 g, 1.86 mmol) in dimethylformamide (25 mL) was added sodium bicarbonate (0.3 g, 3.7 mmol). The mixture was stirred for 16 hours at 80° C., cooled to room temperature, and diluted with ethyl acetate (120 mL). The solution was washed with water, brine, dried over sodium sulfate, and concentrated. The residue was purified by silica gel column (40 g silica gel column, eluting with 0-85% ethyl acetate in petroleum ether) to give the title compound (0.95 g, 58%) as solid. MS (ESI): 800 m/z [M+H]⁺, retention time: 1.93 minutes, purity: 87% (254 nm) (LC-MS method 2).

Ethyl (E)-3-[3-(24,30-difluoro-6-methyl-26-oxa-3,13,15,21,33-pentazahexacyclo-[25.3.1.12,5.112,15.017,25.018,22]tritriaconta-1(31),2,4,12(32),13,17,19,22,24,27,29-undecaen-10-yn-6-yl)phenyl]prop-2-enoate

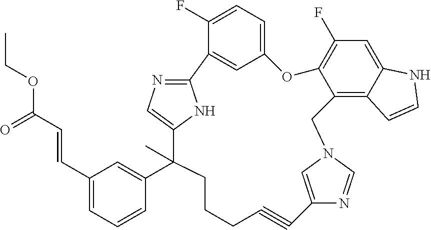 [SMILES: CCOC(=O)/C=C/c1cccc(C2(C)CCCC#Cc3cn(cn3)Cc3c(c(F)cc4[nH]ccc34)Oc3ccc(F)c(c3)-c3ncc2[nH]3)c1]

Step B: To a stirred solution of Step A product (0.7 g, 0.89 mmol) and triethylamine (0.2 mL, 1.34 mmol) in dimethylformamide (25 mL) was added tetrakis(triphenylphosphine)palladium (0) (0.2 g, 0.18 mmol), copper (I) iodide (17 mg, 0.089 mmol). The reaction mixture was heated at 90° C. for 16 hours, cooled to room temperature, and diluted with ethyl acetate (100 mL). The mixture was washed with water, brine, dried over sodium sulfate and concentrated. The residue was purified by automated flash chromatography (20 g silica gel column, eluting with 0-100% ethyl acetate in petroleum ether) to give the title compound (75 mg, 14%) as solid. MS (ESI): 658 m/z [M+H]⁺, retention time: 1.57 minutes, purity: 85% (254 nm) (LC-MS method 2).

Ethyl (E)-3-[3-(24,30-difluoro-6-methyl-26-oxa-3,13,15,21,33-pentazahexacyclo-[25.3.1.12,5.112,15.017,25.018,22]tritriaconta-1(31),2,4,12(32),13,17,19,22,24,27,29-undecaen-10-yn-6-yl)phenyl]prop-2-enoate

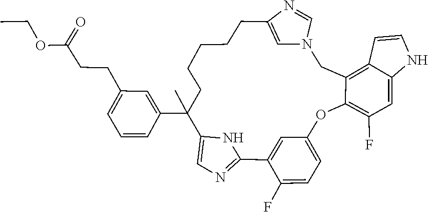 [SMILES: CCOC(=O)CCc1cccc(C2(C)CCCCCc3cn(cn3)Cc3c(c(F)cc4[nH]ccc34)Oc3ccc(F)c(c3)-c3ncc2[nH]3)c1]

Step C: To a stirred solution of Step B product (75 mg; 0.11 mmol) in methanol (10 mL) was added 10% Pd on carbon (50% wet, 60 mg). The mixture was stirred under hydrogen balloon at 35° C. for 6 hours. The mixture was filtered through a pad of Celite. The filtrate was concentrated to give the title compound (35 mg, 50%) as solid. MS (ESI): 664 m/z [M+H]⁺, retention time: 1.46 minutes, purity: 74% (254 nm) (LC-MS method 2).

Compound 53. 3-[3-(24,30-Difluoro-6-methyl-26-oxa-3,13,15,21,33-pentazahexacyclo[25.3.1.12,5.112,15.017,25.018,22]tritriaconta-1(31),2,4,12(32),13,17,19,22,24,27,29-undecaen-6-yl)phenyl]propanoic acid

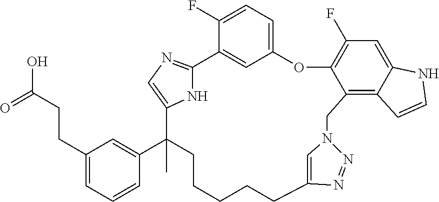 [SMILES: CC1(c2cccc(CCC(=O)O)c2)CCCCCc2cn(nn2)Cc2c(c(F)cc3[nH]ccc23)Oc2ccc(F)c(c2)-c2ncc1[nH]2]

Step D: To a stirred solution of Step C product (35 mg, 0.053 mmol) in tetrahydrofuran (3 mL) and methanol (1 mL) was added a solution of lithium hydroxide (9 mg, 0.2 mmol) in water (1 mL), the reaction was stirred at room temperature for 3 hours, then acidified to pH ˜6 with 1 M hydrochloric acid. The solution was extracted with ethyl acetate (2×20 mL). The combined organic extracts were washed with water, brine, dried over sodium sulfate, and concentrated. The residue was purified by prep-HPLC to give the title compound (16.5 mg, 49%) as white solid. MS (ESI): 636 m/z [M+H]⁺, retention time: 1.39 minutes, purity: 99% (254 nm) (LC-MS method 2). ¹HNMR (400 MHz, CD₃OD). 8.02 (s, 1H), 7.43-7.40 (m 1H), 7.33-7.29 (m, 1H), 7.27-7.20 (m, 2H), 7.15-7.08 (m, 3H), 7.03-6.97 (m, 2H), 6.91-6.89 (m, 1H), 6.74 (s, 1H), 6.70-6.65 (m, 1H), 5.45-6.63 (m, 2H), 2.86-2.80 (m, 2H), 2.53-2.47 (m, 2H), 1.96-2.14 (m, 4H), 1.37-1.47 (m, 6H), 1.16-1.31 (m, 2H), 0.81-0.92 (m, 1H) ppm.

Example 54. Compound 54. 3-[3-(24,30-Difluoro-6,13-dimethyl-26-oxa-3,13,21,32,33-pentazahexacyclo[25.3.1.12,5.112,15.017,25.018,22]tritriaconta-1(31),2,4,12(32),14,17,19,22,24,27,29-undecaen-6-yl)phenyl]propanoic acid

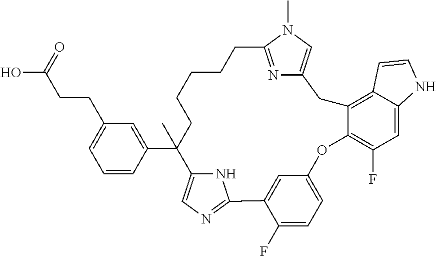 [SMILES: Cn1cc2nc1CCCCCC(C)(c1cccc(CCC(=O)O)c1)c1cnc([nH]1)-c1cc(ccc1F)Oc1c(F)cc3[nH]ccc3c1C2]

7-(2-(2-Fluoro-5-((6-fluoro-4-((3-methyl-2-oxooxazolidin-5-yl)methyl)-1H-indol-5-yl)oxy)phenyl)-1H-imidazol-5-yl)-7-(3-iodophenyl)octanenitrile

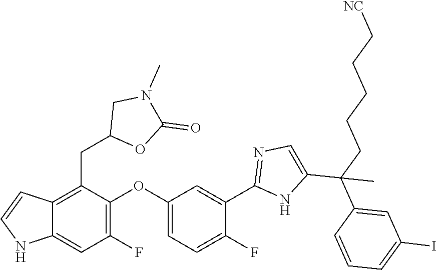 [SMILES: CN1CC(Cc2c(Oc3ccc(F)c(-c4ncc(C(C)(CCCCCC#N)c5cccc(I)c5)[nH]4)c3)c(F)cc3[nH]ccc23)OC1=O]

Step A: To a stirred solution of 2-fluoro-5-((6-fluoro-4-((3-methyl-2-oxooxazolidin-5-yl)methyl)-1H-indol-5-yl)oxy)benzimidamide (Intermediate 6-10, 0.75 g, 1.87 mmol) and 9-chloro-7-(3-iodophenyl)-7-methyl-8-oxononanenitrile (Intermediate 2-32, 0.756 g, 1.87 mmol.) in N,N-dimethylformamide (15 mL) was added sodium bicarbonate (0.32 g, 3.75 mmol). The mixture was heated to 80° C. and stirred for 16 hours, then cooled to room temperature. The mixture was diluted with ethyl acetate (120 mL), washed with water, brine, dried over sodium sulfate and concentrated. The residue was purified by automated silica gel column (40 g silica gel column, eluting with 0-100% ethyl acetate in petroleum ether) to give the title compound (0.75 g, 53%) as a solid. MS (ESI): 750 m/z [M+H]⁺, retention time: 1.88 minutes, purity: 93% (254 nm) (LC-MS method 2).

7-(2-(2-Fluoro-5-((6-fluoro-4-(2-hydroxy-3-(methylamino)propyl)-1H-indol-5-yl)oxy)phenyl)-1H-imidazol-5-yl)-7-(3-iodophenyl)octanoic acid

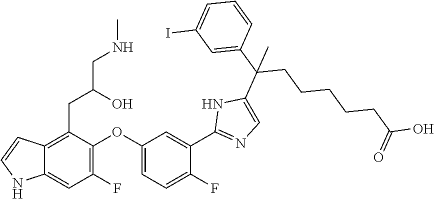 [SMILES: CNCC(O)Cc1c(Oc2ccc(F)c(-c3ncc(C(C)(CCCCCC(=O)O)c4cccc(I)c4)[nH]3)c2)c(F)cc2[nH]ccc12]

Step B: To a stirred solution of Step A product (0.72 g, 0.96 mmol) in water-ethanol (8+8 mL) was added potassium hydroxide (0.8 g, 14.4 mmol). The solution was stirred at 100° C. overnight, then cooled to room temperature and acidified with 1M hydrochloric acid to pH ˜6. The solution was extracted with ethyl acetate-isopropanol (90:10, 3×60 mL). The combined organic phases were dried over sodium sulfate and concentrated to give the title compound (0.61 g, 77.5%) as yellow solid. MS (ESI): 743 m/z [M+H]⁺, retention time: 1.61 minutes, purity: 90% (254 nm) (LC-MS method 2).

Compound 54: 3-[3-(24,30-Difluoro-6,13-dimethyl-26-oxa-3,13,21,32,33-pentazahexacyclo[25.3.1.12,5.112,15.017,25.018,22]tritriaconta-1(31),2,4,12(32),14,17,19,22,24,27,29-undecaen-6-yl)phenyl]propanoic acid

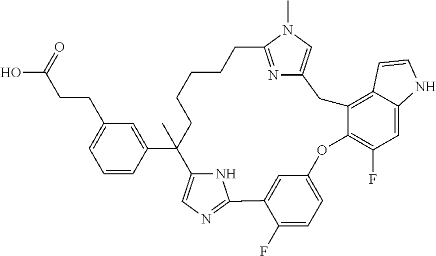 [SMILES: Cn1cc2nc1CCCCCC(C)(c1cccc(CCC(=O)O)c1)c1cnc([nH]1)-c1cc(ccc1F)Oc1c(F)cc3[nH]ccc3c1C2]

Step C: Exchanging (E)-3-(5-(3-(5-(7-Amino-6-hydroxy-2-(3-iodophenyl)heptan-2-yl)-1H-imidazol-2-yl)-4-fluorophenoxy)-6-fluoro-1H-indol-4-yl)acrylic acid (Step C product of Example 21) with 7-(2-(2-Fluoro-5-((6-fluoro-4-(2-hydroxy-3-(methylamino)propyl)-1H-indol-5-yl)oxy)phenyl)-1H-imidazol-5-yl)-7-(3-iodophenyl)octanoic acid (Step B product of this Example, 0.57 g, 0.77 mmol) the reaction procedure sequence (Steps D to G) described for Example 21, followed by the reaction sequence (Steps A to B) of Example 22 were used to prepare the title compound (21 mg) as a white solid. MS (ESI): 650 m/z [M+H]⁺, retention time: 1.46 minutes, purity: 96% (254 nm) (LC-MS method 13). ¹HNMR (400 MHz, CD₃OD). 7.32-7.28 (m, 2H), 7.18-7.11 (m, 4H), 7.01-6.95 (m, 4H), 6.88 (s, 1H), 6.60-6.56 (m, 1H), 4.34-4.19 (m, 2H), 3.42 (s, 3H), 2.84-2.80 (m, 2H), 2.42-2.24 (m, 5H), 2.06-2.00 (m, 1H), 1.70-1.66 (m, 1H), 1.54-1.40 (m, 7H), 0.81-0.77 (m, 1H) ppm.

Example 55. Compound 55. 3-[3-(24,30-Difluoro-6,11,11-trimethyl-10,26-dioxa-3,4,13,14,15,21,33-heptazahexacyclo[25.3.1.12,5.112,15.017,25.018,22]tritriaconta-1(31),2(33),4,12(32),13,17,19,22,24,27,29-undecaen-6-yl)phenyl]propanoic acid

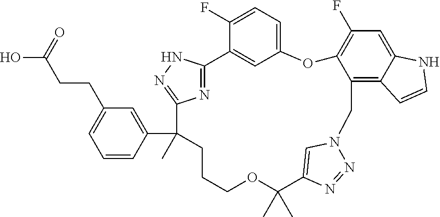 [SMILES: CC1(C)OCCCC(C)(c2cccc(CCC(=O)O)c2)c2n[nH]c(n2)-c2cc(ccc2F)Oc2c(F)cc3[nH]ccc3c2Cn2cc1nn2]

(5-(3-(3-(2-(3-Bromophenyl)-5-((2-methylbut-3-yn-2-yl)oxy)pentan-2-yl)-1H-1,2,4-triazol-5-yl)-4-fluorophenoxy)-6-fluoro-1H-indol-4-yl)methanol

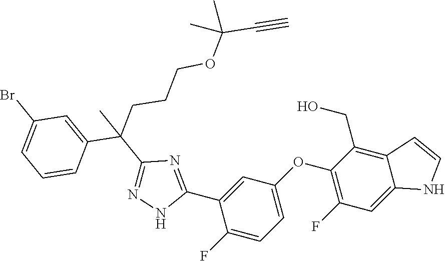 [SMILES: C#CC(C)(C)OCCCC(C)(c1cccc(Br)c1)c1n[nH]c(-c2cc(Oc3c(F)cc4[nH]ccc4c3CO)ccc2F)n1]

Step A: To a stirred solution of 2-fluoro-5-((6-fluoro-4-(hydroxymethyl)-1H-indol-5-yl)oxy)-benzimidamide (Intermediate 6-6, 850 mg, 2.68 mmol) in methanol (10 mL) was added sodium methoxide (5.4 M in methanol) (1.47 mL, 8.04 mmol) and 2-(3-bromophenyl)-2-methyl-5-((2-methylbut-3-yn-2-yl)oxy)pentanehydrazide (Intermediate 45, 1.48 g, 8.04 mmol). The reaction was stirred at 100° C. for 1 hour in a microwave. Additional sodium methoxide (5.4M in methanol) (1 mL, 5.36 mmol) was added and stirred at 100° C. for one more hour in a microwave. The mixture was diluted with ethyl acetate (40 mL) and adjusted pH to ˜5 with 1 M hydrochloric acid. The solution was washed with water, dried over sodium sulfate, and concentrated. The residue was purified by automated flash chromatography (40 g silica gel column, eluting with 0-60% ethyl acetate in petroleum ether) to give the title compound (400 mg, 25.8%) as a solid. MS (ESI): 649 m/z [M+H]⁺, retention time: 2.15 minutes, purity: 86% (214 nm) (LC-MS method 2).

4-(Azidomethyl)-5-(3-(3-(2-(3-bromophenyl)-5-((2-methylbut-3-yn-2-yl)oxy)pentan-2-yl)-1H-1,2,4-triazol-5-yl)-4-fluorophenoxy)-6-fluoro-1H-indole

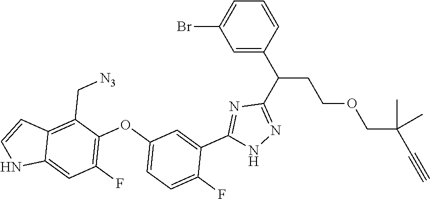 [SMILES: C#CC(C)(C)COCCC(c1cccc(Br)c1)c1n[nH]c(-c2cc(Oc3c(F)cc4[nH]ccc4c3CN=[N+]=[N-])ccc2F)n1]

Step B: To a solution of Step A product (0.42 g, 0.65 mmol) in tetrahydrofuran (10 mL) was added diphenylphosphoryl azide (0.279 mL, 1.29 mmol) and a solution of 1,8-diazabicyclo[5.4.0]undec-7-ene (0.119 g, 0.78 mmol) in tetrahydrofuran (2 mL). The reaction was stirred at 70° C. overnight, cooled to room temperature and quenched with water (30 mL). The solution was extracted with ethyl acetate (3×20 mL). The combined organic extracts were washed with brine (20 mL), dried over sodium sulfate, and concentrated. The residue was purified by automated flash chromatography (40 g silica gel column, eluting with 0-40% ethyl acetate in petroleum ether) to give the title compound (250 mg, 57%) as a solid. MS (ESI): 674 m/z [M+H]⁺, retention time: 2.31 minutes, purity: 87% (214 nm) (LC-MS method 2).

Example 55. 3-[3-(24,30-Difluoro-6,11,11-trimethyl-10,26-dioxa-3,4,13,14,15,21,33-heptazahexacyclo[25.3.1.12,5.112,15.017,25.018,22]tritriaconta-1(31),2(33),4,12(32),13,17,19,22,24,27,29-undecaen-6-yl)phenyl]propanoic acid

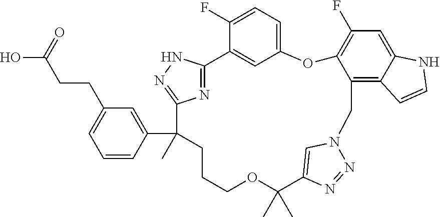 [SMILES: CC1(C)OCCCC(C)(c2cccc(CCC(=O)O)c2)c2n[nH]c(n2)-c2cc(ccc2F)Oc2c(F)cc3[nH]ccc3c2Cn2cc1nn2]

Exchanging 4-(azidomethyl)-6-fluoro-5-(4-fluoro-3-(5-(2-(3-iodophenyl)oct-7-yn-2-yl)-1H-imidazol-2-yl)phenoxy)-1H-indole (Step A product of Example 2) with 4-(Azidomethyl)-5-(3-(3-(2-(3-bromophenyl)-5-((2-methylbut-3-yn-2-yl)oxy)pentan-2-yl)-1H-1,2,4-triazol-5-yl)-4-fluorophenoxy)-6-fluoro-1H-indole (Step B product of this example, 250 mg, 0.37 mmol), the reaction procedure sequence (Steps B to E) described for Example 2 was used to prepare the title compound (69.3 mg) as a white solid. MS (ESI): 668 m/z [M+H]⁺, retention time: 1.65 minutes, purity: >99% (214 nm) (LC-MS method 11). ¹H NMR (400 MHz, CD₃OD) δ 7.51 (s, 1H), 7.47-7.31 (m, 5H), 7.16-6.94 (m, 4H), 6.39-6.25 (m, 1H), 5.97-5.91 (m, 2H), 3.31-3.20 (m, 2H), 3.07-2.91 (m, 1H), 2.82 (t, J=7.7 Hz, 2H), 2.51 (t, J=7.8 Hz, 2H), 2.31-2.24 (m, 1H), 2.00-1.89 (m, 1H), 1.74-1.54 (m, 4H), 1.45-1.38 (m, 6H) ppm.

Example 56. Compound 56A and 56B. Enantiomers 1 and 2 of 3-[3-(24,30-difluoro-6,32-dimethyl-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-6-yl)phenyl]propanoic acid

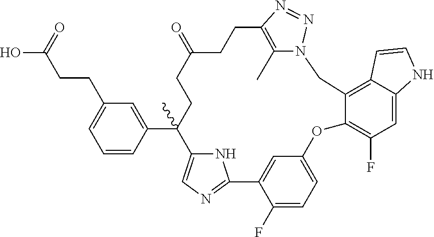 [SMILES: Cc1c2nnn1Cc1c(c(F)cc3[nH]ccc13)Oc1ccc(F)c(c1)-c1ncc([nH]1)C(C)(c1cccc(CCC(=O)O)c1)CCC(=O)CC2]

Exchanging 1-bromo-3-(3-iodophenyl)-3-methylnon-8-yn-2-one (Intermediate 2-51) with 1-bromo-3-(3-iodophenyl)-3-methylundec-9-yn-2-one (Intermediate 2-33, 1.4 g, 3 mmol), the reaction procedure sequence (Steps A to E) described for Example 2 was used to prepare the title compound (178 mg) as a white solid, with slight modification. The catalyst for triazole formation/macrocyclization was switched to chloro(1,5-cyclooctadiene) (pentamethylcyclopentadienyl) ruthenium(II) (10%, with toluene as reaction solvent). The racemic ethyl ester (270 mg), obtained from corresponding Step D of Example 2, was subject to chiral prep-HPLC separation under the following condition: Instrument: Gilson-281, Column: ig 20*250 mm, 10 μm; Mobile phase: n-hexane (0.1% diethylamine): ethanol (0.1% diethylamine)=70:30; Run time per injection: 13 minutes; Injection volume: 0.5 mL; Sample solution: 270 mg in 6.5 mL methanol. The first eluent, enantiomer 1 (35 mg, 13%), was further hydrolyzed to Compound 56A (28 mg, 80%); and the second eluent, Enantiomer 2 (50 mg, 19%), was further hydrolyzed to Compound 56B (47 mg, 94%), both followed the conditions described in Step E of Example 2.

Compound 56A: MS (ESI): 651 m/z [M+H]⁺, retention time: 1.36 minutes, purity: >99% (214 nm) (LC-MS method 2). ¹H NMR (400 MHz, CD₃OD) δ 7.82-6.67 (m, 10H), 6.45 (s, 1H), 5.91-5.76 (m, 2H), 2.87 (t, J=7.5 Hz, 2H), 2.55 (t, J=7.4 Hz, 2H), 2.34-2.00 (m, 6H), 1.71-0.86 (m, 10H) ppm.

Compound 56B: MS (ESI): 651 m/z [M+H]⁺, retention time: 1.36 minutes, purity: >99% (214 nm) (LC-MS method 2). ¹H NMR (400 MHz, CD₃OD) δ 7.82-6.67 (m, 10H), 6.45 (s, 1H), 5.91-5.76 (m, 2H), 2.87 (t, J=7.5 Hz, 2H), 2.55 (t, J=7.4 Hz, 2H), 2.34-2.00 (m, 6H), 1.71-0.86 (m, 10H) ppm.

Example 57. Compound 57. 3-[3-(11,11,24,30-Tetrafluoro-6-methyl-9,26-dioxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

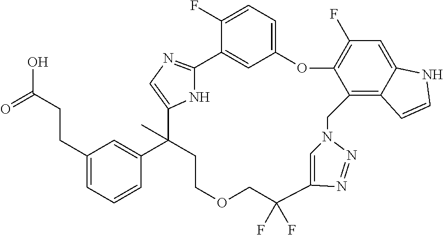 [SMILES: CC1(c2cccc(CCC(=O)O)c2)CCOCC(F)(F)c2cn(nn2)Cc2c(c(F)cc3[nH]ccc23)Oc2ccc(F)c(c2)-c2ncc1[nH]2]

Exchanging 1-bromo-3-(3-iodophenyl)-3-methylnon-8-yn-2-one (Intermediate 2-51) with 1-bromo-5-((2,2-difluorobut-3-yn-1-yl)oxy)-3-(3-iodophenyl)-3-methylpentan-2-one (Intermediate 2-34, 150 mg, 0.266 mmol), the reaction procedure sequence (Steps A to E) described for Example 2 was used to prepare the title compound (2.5 mg) as a white solid. MS (ESI): 675 m/z [M+H]⁺, retention time: 1.37 minutes, purity: >99% (254 nm) (LC-MS method 5). ¹H NMR (400 MHz, CD₃OD) δ 7.67 (s, 1H), 7.28 (d, J=3.2 Hz, 1H), 7.24-7.14 (m, 3H), 7.02-6.82 (m, 6H), 6.59 (d, J=3.1 Hz, 1H), 5.92 (d, J=14.5 Hz, 1H), 5.78 (d, J=14.5 Hz, 1H), 3.77-3.44 (m, 4H), 2.70 (t, J=7.7 Hz, 2H), 2.35 (t, J=7.8 Hz, 2H), 2.25-2.09 (m, 2H), 1.37 (s, 3H) ppm.

Example 58. Compound 58A and 58B. 3-[3-(24,30-Difluoro-6,9,9-trimethyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]propanoic acid

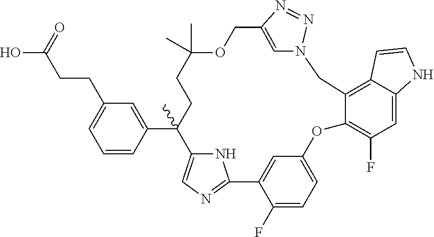 [SMILES: CC1(C)CCC(C)(c2cccc(CCC(=O)O)c2)c2cnc([nH]2)-c2cc(ccc2F)Oc2c(F)cc3[nH]ccc3c2Cn2cc(nn2)CO1]

Exchanging 1-bromo-3-(3-iodophenyl)-3-methylnon-8-yn-2-one (Intermediate 2-51) with 1-bromo-3-(3-iodophenyl)-3,6-dimethyl-6-(prop-2-yn-1-yloxy)heptan-2-one (Intermediate 2-35, 1.02 g, 2.14 mmol), the reaction procedure sequence (Steps A to E) described for Example 2 was used to prepare the title compounds. The racemic ethyl acrylates (520 mg), obtained from corresponding Step C of Example 2, was separable under chiral SFC separation conditions. The first eluent (200 mg, 46%), enantiomer 1, underwent hydrogenation and hydrolysis to afford Compound 58A (178 mg, 77% two steps); The second eluent (192 mg, 44%), enantiomer 2, also underwent hydrogenation and hydrolysis to afford Compound 58B (140 mg, 76% two steps), following conditions described in Step D and E of Example 2.

Compound 58A: MS (ESI): 667 m/z [M+H]⁺, retention time: 1.36 minutes, purity: >99% (254 nm) (LC-MS method 8). ¹H NMR (400 MHz, CD₃OD) δ 7.52 (s, 1H), 7.26 (d, J=3.2 Hz, 1H), 7.19 (d, J=10.6 Hz, 1H), 7.08-6.88 (m, 6H), 6.87-6.80 (m, 1H), 6.75-6.71 (m, 1H), 6.50-6.44 (m, 1H), 5.87 (d, J=14.4 Hz, 1H), 5.64 (d, J=14.4 Hz, 1H), 3.97 (d, J=10.9 Hz, 1H), 3.63 (t, J=9.8 Hz, 1H), 2.67 (t, J=7.7 Hz, 2H), 2.36 (t, J=7.7 Hz, 2H), 2.18-2.11 (m, 1H), 1.98 (dt, J=12.3, 6.2 Hz, 1H), 1.37 (s, 3H), 1.20-1.26 (m, 2H), 1.07 (s, 3H), 1.04 (s, 3H) ppm.

Compound 58B: MS (ESI): 667 m/z [M+H]⁺, retention time: 1.36 minutes, purity: >99% (254 nm) (LC-MS method 8). ¹H NMR (400 MHz, CD₃OD) δ 7.52 (s, 1H), 7.26 (d, J=3.2 Hz, 1H), 7.19 (d, J=10.6 Hz, 1H), 7.08-6.87 (m, 6H), 6.87-6.80 (m, 1H), 6.75-6.71 (m, 1H), 6.50-6.44 (m, 1H), 5.87 (d, J=14.4 Hz, 1H), 5.64 (d, J=14.4 Hz, 1H), 3.97 (d, J=10.9 Hz, 1H), 3.63 (t, J=9.8 Hz, 1H), 2.67 (t, J=7.7 Hz, 2H), 2.36 (t, J=7.7 Hz, 2H), 2.18-2.10 (m, 1H), 1.98 (dt, J=12.3, 6.2 Hz, 1H), 1.37 (s, 3H), 1.20-1.26 (m, 2H), 1.07 (s, 3H), 1.05 (s, 3H) ppm.

Example 59. Compound 59. Ethyl 3-[3-(24,30-difluoro-6,11,11-trimethyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]butanoate

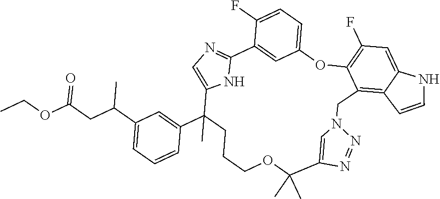 [SMILES: CCOC(=O)CC(C)c1cccc(C2(C)CCCOC(C)(C)c3cn(nn3)Cc3c(c(F)cc4[nH]ccc34)Oc3ccc(F)c(c3)-c3ncc2[nH]3)c1]

and

Example 60. Compound 60. 24,30-difluoro-6,11,11-trimethyl-6-phenyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaene

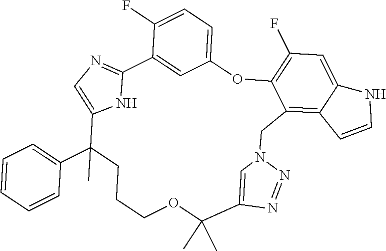 [SMILES: CC1(C)OCCCC(C)(c2ccccc2)c2cnc([nH]2)-c2cc(ccc2F)Oc2c(F)cc3[nH]ccc3c2Cn2cc1nn2]

6-(3-Bromophenyl)-24,30-difluoro-6,11,11-trimethyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaene

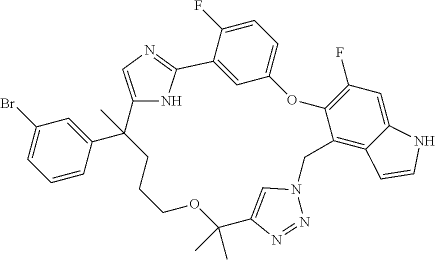 [SMILES: CC1(C)OCCCC(C)(c2cccc(Br)c2)c2cnc([nH]2)-c2cc(ccc2F)Oc2c(F)cc3[nH]ccc3c2Cn2cc1nn2]

Step A: Exchanging 1-bromo-3-(3-iodophenyl)-3-methylnon-8-yn-2-one (Intermediate 2-51) with 3-(3-bromophenyl)-1-chloro-3-methyl-6-((2-methylbut-3-yn-2-yl)oxy)hexan-2-one (Intermediate 2-30,460 mg, 1.2 mmol), the reaction procedure sequence (Steps A to B) described for Example 2 was used to prepare the title compound (320 mg) as a yellow solid. MS (ESI): 673, 675 m/z [M+H]⁺.

Compound 59.-Ethyl 3-[3-(24,30-difluoro-6,11,11-trimethyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]butanoate

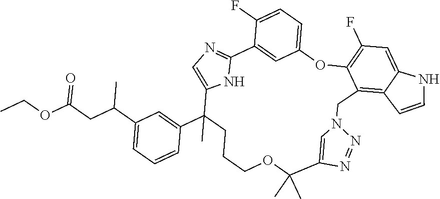 [SMILES: CCOC(=O)CC(C)c1cccc(C2(C)CCCOC(C)(C)c3cn(nn3)Cc3c(c(F)cc4[nH]ccc34)Oc3ccc(F)c(c3)-c3ncc2[nH]3)c1]

Compound 60. 24,30-Difluoro-6,11,11-trimethyl-6-phenyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaene

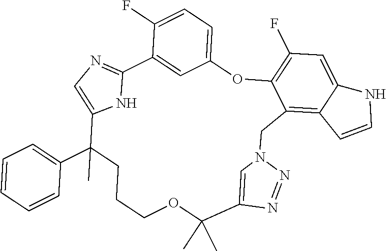 [SMILES: CC1(C)OCCCC(C)(c2ccccc2)c2cnc([nH]2)-c2cc(ccc2F)Oc2c(F)cc3[nH]ccc3c2Cn2cc1nn2]

Step B: Exchanging 23,29-difluoro-6-(3-iodophenyl)-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaene (Step B product of Example 2) with 6-(3-Bromophenyl)-24,30-difluoro-6,11,11-trimethyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaene (Step A product of this Example, 900 mg, 1.3 mmol), and ethyl acrylate with ethyl (E)-but-2-enoate (296 mg, 2.6 mmol), the reaction procedure sequence (Steps C and D) described for Example 2 was used to prepare the title compounds. Two products, 59 (154 mg, originated from Heck coupling product of corresponding Step C), and 60 (44 mg, originated from debromination product of corresponding Step C), were separated at corresponding Step D by prep-HPLC.

Compound 59: MS (ESI): 709 m/z [M+H]⁺, retention time: 1.77 minutes, purity: >99% (254 nm) (LC-MS method 29). ¹H NMR (400 MHz, CD₃OD) δ 7.51 (s, 1H), 7.35-7.25 (m, 5H), 7.13-6.95 (m, 5H), 6.35 (s, 1H), 5.95-5.82 (m, 2H), 3.98-3.88 (m, 2H), 3.26-3.02 (m, 2H), 2.95-2.88 (m, 1H), 2.48 (d, J=7.2 Hz, 2H), 2.29-2.05 (m, 2H), 1.94-1.86 (m, 1H), 1.49 (s, 3H), 1.42 (s, 3H), 1.38-1.30 (m, 4H), 1.19 (d, J=6.8 Hz, 3H), 1.09-1.03 (m, 3H).

Compound 59: MS (ESI): 595 m/z [M+H]⁺, retention time: 1.67 minutes, purity: >99% (254 nm) (LC-MS method 29). ¹H NMR (400 MHz, DMSO-d₆) δ 11.90 (s, 1H), 11.45 (s, 1H), 7.47-7.01 (m, 11H), 6.93 (d, J=2.0 Hz, 1H), 6.18 (s, 1H), 5.90-5.77 (m, 2H), 3.18-3.11 (m, 1H), 2.92-2.85 (m, 1H), 2.20-2.11 (m, 1H), 2.04-1.93 (m, 1H), 1.66-1.58 (m, 1H), 1.47 (s, 3H), 1.35 (s, 3H), 1.256 (s, 3H), 1.08-1.01 (m, 1H).

Example 61. Compound 61. Ethyl 3-[3-(24,30-difluoro-6,11,11-trimethyl-10,26-dioxa-3,13,14,15,21,33-hexazahexacyclo[25.3.1.12,5.112,15.017,25.018,22]tritriaconta-1(31),2,4,12(32),13,17,19,22,24,27,29-undecaen-6-yl)phenyl]-2-methyl-propanoate

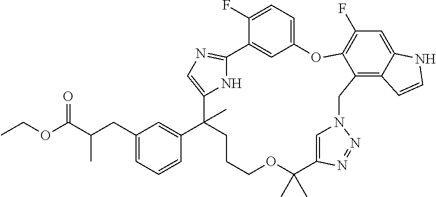

Exchanging ethyl (E)-but-2-enoate with ethyl methacrylate (296 mg, 2.6 mmol), the reaction procedure sequence (Step B) described for example 59 was used to prepare the title compound as Example 61 (108 mg). MS (ESI): 709 m/z [M+H]⁺, retention time: 1.45 minutes, purity: 98% (214 nm) (LC-MS method 30). ¹H NMR (400 MHz, CD₃OD) δ 7.50-7.25 (m, 6H), 7.13-6.85 (m, 5H), 6.35 (s, 1H), 5.95-5.82 (m, 2H), 3.98-3.90 (m, 2H), 3.18-3.16 (m, 1H), 2.96-2.74 (m, 2H), 2.64-2.58 (m, 2H), 2.25-1.87 (m, 3H), 1.48 (s, 3H), 1.42 (s, 3H), 1.38-1.30 (m, 4H), 1.13-1.01 (m, 6H).

Example 62. Compound 62A and 62B. Enantiomer 1 and 2 of 3-[3-(24,30-Difluoro-6,11,11-trimethyl-26,32-dioxa-3,14,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

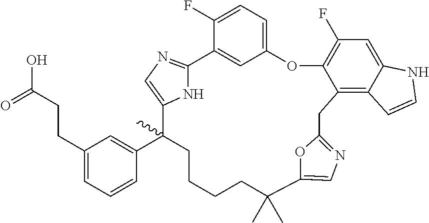 [SMILES: CC1(C)CCCCC(C)(c2cccc(CCC(=O)O)c2)c2cnc([nH]2)-c2cc(ccc2F)Oc2c(F)cc3[nH]ccc3c2CC2=NC=C1O2]

Ethyl 2-(6-fluoro-5-(4-fluoro-3-(5-(2-(3-iodophenyl)-7-methyl-7-(2-oxooxazolidin-5-yl)octan-2-yl)-1H-imidazol-2-yl)phenoxy)-1H-indol-4-yl)acetate

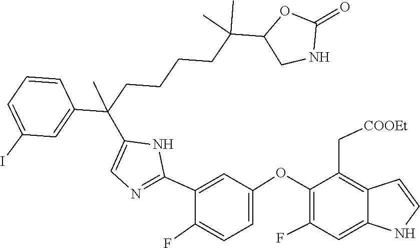 [SMILES: CCOC(=O)Cc1c(Oc2ccc(F)c(-c3ncc(C(C)(CCCCC(C)(C)C4CNC(=O)O4)c4cccc(I)c4)[nH]3)c2)c(F)cc2[nH]ccc12]

Step A: To a solution of 5-(9-bromo-7-(3-iodophenyl)-2,7-dimethyl-8-oxononan-2-yl)oxazolidin-2-one (Intermediate 2-36, 1.19 g, 3.19 mmol) in dimethylformamide (30 mL) was added ethyl 2-(5-(3-carbamimidoyl-4-fluorophenoxy)-6-fluoro-1H-indol-4-yl)acetate (Intermediate 29, 1.19 g, 3.19 mmol) and sodium bicarbonate (893 mg, 10.6 mmol). The reaction was stirred at 70° C. for 18 hours, cooled to room temperature. The mixture was partitioned between water (100 mL) and ethyl acetate (100 mL). The separated organic layer, combined with two additional ethyl acetate extracts (2×50 mL), was dried over sodium sulfate, and concentrated. The residue was purified by automated flash chromatography eluting with 100% ethyl acetate to methanol/dichloromethane=1/20 to afford the title compound (2.4 g, 84%) as a light-yellow oil. MS (ESI): 811 m/z [M+H]⁺, retention time: 1.75 minutes, purity: 80% (254 nm) (LC-MS method 6).

2-(5-(3-(5-(9-Amino-8-hydroxy-2-(3-iodophenyl)-7,7-dimethylnonan-2-yl)-1H-imidazol-2-yl)-4-fluorophenoxy)-6-fluoro-1H-indol-4-yl)acetic acid

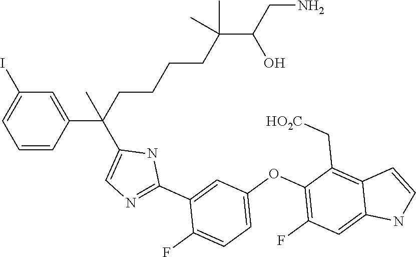

Step B: To a solution of Step A product (2.4 g, 2.96 mmol) in methanol (100 mL) and water (10 mL) was added potassium hydroxide (3.32 g, 59.3 mmol). The mixture was stirred at 80° C. for 48 hours, then concentrated to remove methanol. The residue was diluted with water (50 mL), acidified with 2 N hydrochloric acid to pH ˜6, and extracted with a solution of dichloromethane/i-PrOH=1/10 (3×100 mL). The combined organic layers were dried over sodium sulfate and concentrated to afford the title compound (2.0 g, 89%) as light red oil. MS (ESI): 757 m/z [M+H]⁺, retention time: 1.50 minutes, purity: 90% (254 nm) (LC-MS method 6).

Compounds 62A and 62B: 3-[3-(24,30-Difluoro-6,11,11-trimethyl-26,32-dioxa-3,14,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

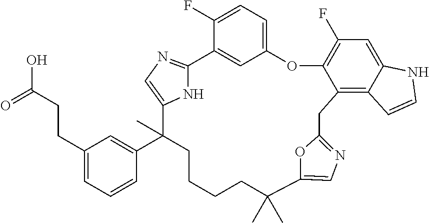 [SMILES: CC1(C)CCCCC(C)(c2cccc(CCC(=O)O)c2)c2cnc([nH]2)-c2cc(ccc2F)Oc2c(F)cc3[nH]ccc3c2CC2=NC=C1O2]

Exchanging (E)-3-(5-(3-(5-(7-Amino-6-hydroxy-2-(3-iodophenyl)heptan-2-yl)-1H-imidazol-2-yl)-4-fluorophenoxy)-6-fluoro-1H-indol-4-yl)acrylic acid (Step C product of Example 21) with 2-(5-(3-(5-(9-Amino-8-hydroxy-2-(3-iodophenyl)-7,7-dimethylnonan-2-yl)-1H-imidazol-2-yl)-4-fluorophenoxy)-6-fluoro-1H-indol-4-yl)acetic acid (Step B product of this example, 2.0 g, 2.64 mmol), the reaction procedure sequence (Steps D to I) described for Example 21 was used to prepare the title compounds. The racemic methyl ester (180 mg) from corresponding Step H of Example 21 was subjected to chiral SFC separation. The first eluent (50 mg, 28%), enantiomer 1, was further hydrolyzed to Compound 62A (38 mg, 78%, light yellow solid); The second eluent (47 mg, 26%), enantiomer 2, was further hydrolyzed to Compound 62B (38 mg, 83%, light yellow solid), following the conditions described in Step I of Example 21

Compound 62A: MS (ESI): 665 m/z [M+H]⁺, retention time: 1.71 minutes, purity: 96% (254 nm) (LC-MS method 6). ¹H NMR (400 MHz, CD₃OD) δ 7.34-7.30 (m, 2H), 7.29-7.26 (m, 2H), 7.23-7.13 (m, 3H), 7.08-7.06 (m, 2H), 7.02-7.00 (m, 1H), 6.55-6.51 (m, 1H), 6.46 (s, 1H), 4.29-4.23 (m, 2H), 2.80-2.76 (m, 2H), 2.58-2.54 (m, 2H), 2.23-2.05 (m, 2H), 1.56 (s, 3H), 1.52-1.21 (m, 4H), 1.16 (s, 3H), 1.07 (s, 3H), 0.94-0.85 (m, 2H).

Compound 62B: MS (ESI): 665 m/z [M+H]⁺, retention time: 1.71 minutes, purity: >99% (254 nm) (LC-MS method 6). ¹H NMR (400 MHz, CD₃OD) δ 7.38 (s, 1H), 7.35-7.30 (m, 1H), 7.30-7.26 (m, 2H), 7.24-7.19 (m, 2H), 7.14 (s, 1H), 7.09-7.05 (m, 2H), 7.00-6.97 (m, 1H), 6.55-6.51 (m, 1H), 6.45 (s, 1H), 4.29-4.23 (m, 2H), 2.80-2.76 (m, 2H), 2.58-2.54 (m, 2H), 2.30-2.02 (m, 2H), 1.57 (s, 3H), 1.54-1.20 (m, 4H), 1.17 (s, 3H), 1.07 (s, 3H), 0.99-0.80 (m, 2H).

Example 63. Compound 63. 3-[3-(24,30-Difluoro-6,11,11-trimethyl-26-oxa-32-thia-3,14,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

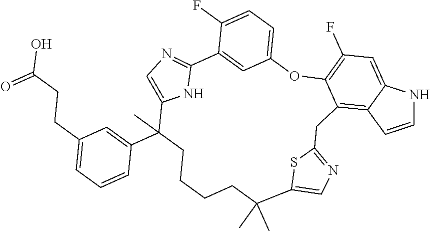 [SMILES: CC1(C)CCCCC(C)(c2cccc(CCC(=O)O)c2)c2cnc([nH]2)-c2cc(ccc2F)Oc2c(F)cc3[nH]ccc3c2CC2=NC=C1S2]

Exchanging (E)-3-(5-(3-(5-(7-Amino-6-hydroxy-2-(3-iodophenyl)heptan-2-yl)-1H-imidazol-2-yl)-4-fluorophenoxy)-6-fluoro-1H-indol-4-yl)acrylic acid (Step C product of Example 21) with 2-(5-(3-(5-(9-Amino-8-hydroxy-2-(3-iodophenyl)-7,7-dimethylnonan-2-yl)-1H-imidazol-2-yl)-4-fluorophenoxy)-6-fluoro-1H-indol-4-yl)acetic acid (Step B product of Example 62, 2.0 g, 2.64 mmol), the reaction procedure sequence (Steps D to G) described for Example 21, followed by reaction procedure sequence (Steps A to B) described for Example 23, were used to prepare the title compound (7 mg, white solid). MS (ESI): 681 m/z [M+H]⁺, retention time: 1.71 minutes, purity: >99% (254 nm) (LC-MS method 6). ¹H NMR (400 MHz, CD₃OD) δ 7.31 (d, 1H), 7.24-7.12 (m, 6H), 7.05-6.99 (m, 4H), 6.56 (d, 1H), 4.56-4.48 (m, 2H), 2.86-2.82 (m, 2H), 2.53-2.49 (m, 2H), 2.11-2.02 (m, 2H), 1.48 (s, 3H), 1.48-1.28 (m, 2H), 1.21 (s, 3H), 1.17 (s, 3H), 1.26-1.03 (m, 2H), 0.94-0.83 (m, 2H).

Example 64. Compound 64. 3-[3-(24,30-difluoro-6,9-dimethyl-14,26-dioxa-3,21,32,33-tetrazahexacyclo[25.3.1.12,5.112,15.017,25.018,22]tritriaconta-1(31),2,4,12,15(32),17,19,22,24,27,29-undecaen-6-yl)phenyl]propanoic acid

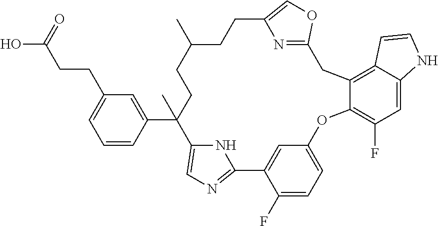 [SMILES: CC1CCc2coc(n2)Cc2c(c(F)cc3[nH]ccc23)Oc2ccc(F)c(c2)-c2ncc([nH]2)C(C)(c2cccc(CCC(=O)O)c2)CC1]

Exchanging 5-(9-bromo-7-(3-iodophenyl)-2,7-dimethyl-8-oxononan-2-yl)oxazolidin-2-one (Intermediate 2-36) with 4-(8-bromo-6-(3-iodophenyl)-3,6-dimethyl-7-oxooctyl)oxazolidin-2-one (Intermediate 2-37, 1.7 g, 0.00326 mol), the reaction procedure sequence (Steps A to C) described in the Example 62A and 62B was used to prepare the title compound (18 mg) as an inseparable mixture of 4 diastereomers, originating from two chiral centers. MS (ESI): 651 m/z [M+H]⁺, retention time: 1.60 minutes, purity: >99% (214 nm) (LC-MS method 6).

Example 65. Compound 65. 3-[3-(24,30-Difluoro-6,11,11-trimethyl-10,26-dioxa-32-thia-3,14,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

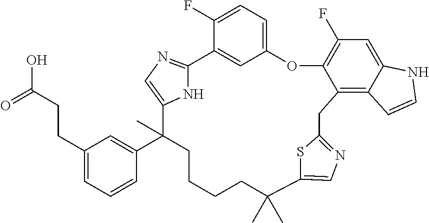 [SMILES: CC1(C)CCCCC(C)(c2cccc(CCC(=O)O)c2)c2cnc([nH]2)-c2cc(ccc2F)Oc2c(F)cc3[nH]ccc3c2CC2=NC=C1S2]

Ethyl 2-(5-(3-(5-(5-((3-acetoxy-4-(((benzyloxy)carbonyl)amino)-2-methylbutan-2-yl)oxy)-2-(3-iodophenyl)pentan-2-yl)-1H-imidazol-2-yl)-4-fluorophenoxy)-6-fluoro-1H-indol-4-yl)acetate

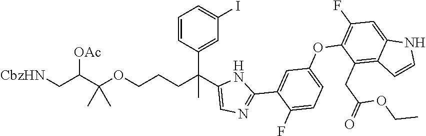 [SMILES: CCOC(=O)Cc1c(Oc2ccc(F)c(-c3ncc(C(C)(CCCOC(C)(C)C(CNC(=O)OCc4ccccc4)OC(C)=O)c4cccc(I)c4)[nH]3)c2)c(F)cc2[nH]ccc12]

Step A: to a stirred solution of ethyl 2-(5-(3-carbamimidoyl-4-fluorophenoxy)-6-fluoro-1H-indol-4-yl)acetate (Intermediate 29, 2.28 g, 6.1 mmol) and 1-(((benzyloxy)carbonyl)amino)-3-((6-bromo-4-(3-iodophenyl)-4-methyl-5-oxohexyl)oxy)-3-methylbutan-2-yl acetate (Intermediate 2-38, 4 g, 5.81 mmol) in dimethylformamide (50 mL) was added sodium bicarbonate (0.98 g, 11.7 mmol). The mixture was stirred at 75° C. overnight, cooled to room temperature and quenched with water (40 mL). The solution was extracted with ethyl acetate (3×50 mL). The combined organic extracts were washed with brine, dried over sodium sulfate, and concentrated. The residue was purified by automated flash chromatography (80 g silica gel column, eluting with 0-80% ethyl acetate in petroleum ether) to give the title compound (2.5 g, 44%) as a solid. MS (ESI): 963 m/z [M+H]⁺, retention time: 1.93 minutes, purity: 81% (214 nm) (LC-MS method 2).

2-(6-Fluoro-5-(4-fluoro-3-(5-(2-(3-iodophenyl)-5-((2-(2-oxooxazolidin-5-yl)propan-2-yl)oxy)pentan-2-yl)-1H-imidazol-2-yl)phenoxy)-1H-indol-4-yl)acetic acid

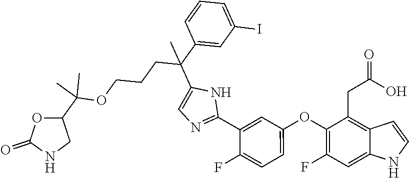 [SMILES: CC(CCCOC(C)(C)C1CNC(=O)O1)(c1cccc(I)c1)c1cnc(-c2cc(Oc3c(F)cc4[nH]ccc4c3CC(=O)O)ccc2F)[nH]1]

Step B: To a stirred solution of Step A product (2.5 g, 2.6 mmol) in tetrahydrofuran/water/methanol (20 mL/20 mL/20 mL) was added lithium hydroxide monohydrate (1.09 g, 26 mmol). The reaction was stirred at room temperature overnight. The residue was acidified with 1 M hydrochloric acid to pH ˜4 and extracted with dichloromethane/isopropanol (10:1, 5×30 mL). The combined organic extracts were dried over sodium sulfate and concentrated. The residue was purified by automated flash chromatography (20 g silica gel column, eluting with 0-10% methanol 1 in dichloromethane) to give the title compound (1.85 g, 90%) as a solid. MS (ESI): 785 m/z [M+H]⁺, retention time: 1.74 minutes, purity: 82% (214 nm) (LC-MS method 2).

2-(5-(3-(5-(5-((4-amino-3-hydroxy-2-methylbutan-2-yl)oxy)-2-(3-iodophenyl)pentan-2-yl)-1H-imidazol-2-yl)-4-fluorophenoxy)-6-fluoro-1H-indol-4-yl)acetic acid

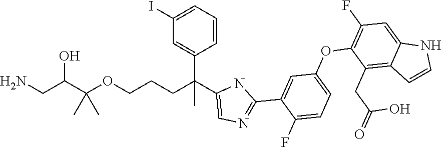 [SMILES: CC(CCCOC(C)(C)C(O)CN)(c1cccc(I)c1)c1cnc(-c2cc(Oc3c(F)cc4[nH]ccc4c3CC(=O)O)ccc2F)[nH]1]

Step C: To a stirred solution of Step B product (1.85 g, 2.36 mmol) in tetrahydrofuran/water/methanol (8 mL/8 mL/8 mL) was added lithium hydroxide monohydrate (1.98 g, 47.2 mmol). The reaction was stirred at 60° C. overnight. The mixture was acidified with 1M hydrochloric acid to pH ˜6, extracted with dichloromethane/isopropanol (10:1, 5×30 mL). The combined organic extracts were dried over sodium sulfate, and concentrated. The crude product (1.6 g, 90%) was used for next step without further purification. MS (ESI): 759 m/z [M+H]⁺, retention time: 1.67 minutes, purity: 88% (214 nm) (LC-MS method 2).

Compound 65: 3-[3-(24,30-Difluoro-6,11,11-trimethyl-10,26-dioxa-32-thia-3,14,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

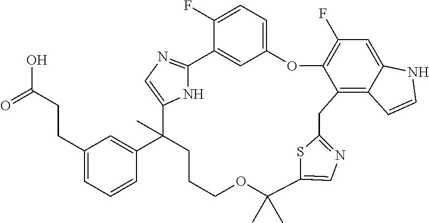 [SMILES: CC1(C)OCCCC(C)(c2cccc(CCC(=O)O)c2)c2cnc([nH]2)-c2cc(ccc2F)Oc2c(F)cc3[nH]ccc3c2CC2=NC=C1S2]

Step D: Exchanging (E)-3-(5-(3-(5-(7-Amino-6-hydroxy-2-(3-iodophenyl)heptan-2-yl)-1H-imidazol-2-yl)-4-fluorophenoxy)-6-fluoro-1H-indol-4-yl)acrylic acid (Step C product of Example 21) with 2-(5-(3-(5-(5-((4-amino-3-hydroxy-2-methylbutan-2-yl)oxy)-2-(3-iodophenyl)pentan-2-yl)-1H-imidazol-2-yl)-4-fluorophenoxy)-6-fluoro-1H-indol-4-yl)acetic acid (Step C product of this example, 1.6 g, 2.11 mmol), the reaction procedure sequence (Steps D to G) described for Example 21, followed by reaction procedure sequence (Steps A and B) described for Example 23, were used to prepare the title compound (1.7 mg) as a racemic mixture. MS (ESI): 683 m/z [M+H]⁺, retention time: 1.36 minutes, purity: 96% (214 nm) (LC-MS method 2). ¹H NMR (400 MHz, CD₃OD) δ 7.26-6.98 (m, 11H), 6.44-6.43 (m, 1H), 4.61 (d, J=15.8 Hz, 1H), 4.49 (d, J=15.8 Hz, 1H), 3.20-2.93 (m, 2H), 2.94-2.81 (m, 2H), 2.49-2.45 (m, 2H), 2.22-1.97 (m, 2H), 1.51-1.48 (m, 7H), 1.39 (s, 3H), 1.29-1.19 (m, 1H).

Example 66. Compound 66. 3-[3-(24,30-Difluoro-6,11,11-trimethyl-10,26,32-trioxa-3,14,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

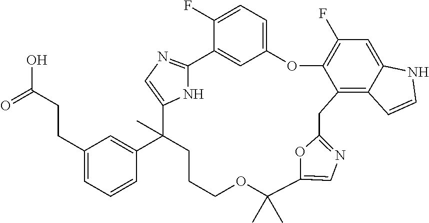 [SMILES: CC1(C)OCCCC(C)(c2cccc(CCC(=O)O)c2)c2cnc([nH]2)-c2cc(ccc2F)Oc2c(F)cc3[nH]ccc3c2CC2=NC=C1O2]

Exchanging (E)-3-(5-(3-(5-(7-amino-6-hydroxy-2-(3-iodophenyl)heptan-2-yl)-1H-imidazol-2-yl)-4-fluorophenoxy)-6-fluoro-1H-indol-4-yl)acrylic acid (Step C product of Example 21) with 2-(5-(3-(5-(5-((4-amino-3-hydroxy-2-methylbutan-2-yl)oxy)-2-(3-iodophenyl)pentan-2-yl)-1H-imidazol-2-yl)-4-fluorophenoxy)-6-fluoro-1H-indol-4-yl)acetic acid (Step C product of Example 65, 1.6 g, 2.11 mmol), the reaction procedure sequence (Steps D to I) described for Example 21 was used to prepare the title compound (7.3 mg) as a racemic mixture. MS (ESI): 667 m/z [M+H]⁺, retention time: 1.71 minutes, purity: 95% (214 nm) (LC-MS method 2). 1H NMR (400 MHz, CD₃OD) δ7.33-7.09 (m, 6H), 7.07-6.92 (m, 4H), 6.68 (s, 1H), 6.55 (d, J=2.5 Hz, 1H), 4.34-4.30 (m, 2H), 2.93-2.82 (m, 3H), 2.70-2.60 (m, 1H), 2.53 (t, J=7.8 Hz, 2H), 2.14-1.98 (m, 2H), 1.53-1.28 (m, 11H).

Example 67. Compound 67A, 67B. 3-[3-(24,30-Difluoro-6,11,11-trimethyl-26-oxa-3,13,14,15,19,21,33-heptazahexacyclo[25.3.1.12,5.112,15.017,25.018,22]tritriaconta-1(31),2,4,12(32),13,17,19,22,24,27,29-undecaen-6-yl)phenyl]propanoic acid

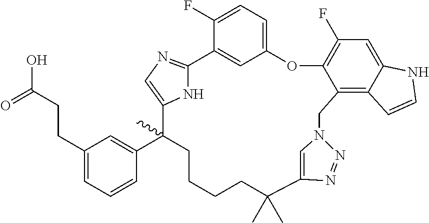 [SMILES: CC1(C)CCCCC(C)(c2cccc(CCC(=O)O)c2)c2cnc([nH]2)-c2cc(ccc2F)Oc2c(F)cc3[nH]ccc3c2Cn2cc1nn2]

and

Example 68. Compound 68. 24,30-Difluoro-6,11,11-trimethyl-6-phenyl-26-oxa-3,13,14,15,19,21,33-heptazahexacyclo[25.3.1.12,5.112,15.017,25.018,22]tritriaconta-1(31),2,4,12(32),13,17,19,22,24,27,29-undecaene

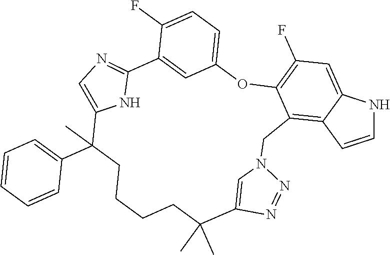 [SMILES: CC1(C)CCCCC(C)(c2ccccc2)c2cnc([nH]2)-c2cc(ccc2F)Oc2c(F)cc3[nH]ccc3c2Cn2cc1nn2]

(5-(3-(5-(2-(3-Bromophenyl)-7,7-dimethylnon-8-yn-2-yl)-1H-imidazol-2-yl)-4-fluorophenoxy)-6-fluoro-1-(tetrahydro-2H-pyran-2-yl)-1H-benzo[d]imidazol-4-yl)methanol

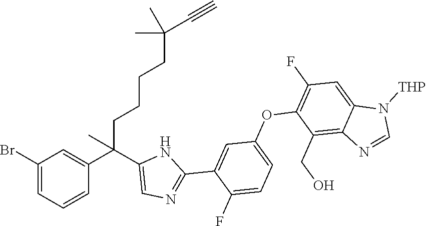

Step A: To a stirred solution of 1-bromo-3-(3-bromophenyl)-3,8,8-trimethyldec-9-yn-2-one (Intermediate 2-39, 2.93 g, 6.83 mmol) and 2-fluoro-5-((6-fluoro-4-(hydroxymethyl)-1-(tetrahydro-2H-pyran-2-yl)-1H-benzo[d]imidazol-5-yl)oxy)benzimidamide (Intermediate 6-11, 2.75 g, 6.83 mmol) in dimethylformamide (60 mL) was added sodium bicarbonate (1.15 g, 13.7 mmol). The reaction was stirred at 80° C. overnight, diluted with water (50 mL), and extracted with ethyl acetate (3×50 mL). The combined organic extracts were washed with brine, dried over sodium sulfate, and concentrated. The residue was purified by automated flash chromatography (40 g silica gel column, eluting with 0-100% ethyl acetate in petroleum ether) to give the title compound (3 g, 60%) as a solid. MS (ESI): 731 m/z [M+H]⁺, retention time: 1.94 minutes, purity: 90% (254 nm) (LC-MS method 2).

4-(Azidomethyl)-5-(3-(5-(2-(3-bromophenyl)-7,7-dimethylnon-8-yn-2-yl)-1H-imidazol-2-yl)-4-fluorophenoxy)-6-fluoro-1-(tetrahydro-2H-pyran-2-yl)-1H-benzo[d]imidazole

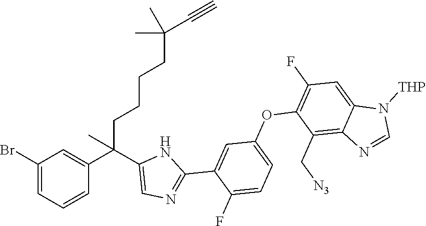

Step B: To a stirred solution of Step A product (3 g, 4.1 mmol) in tetrahydrofuran (100 mL) was added diphenylphosphoryl azide (2.26 g, 8.2 mmol) and 1,8-diazabicyclo[5.4.0]undec-7-ene (639 μL, 5 mmol). The reaction was refluxed overnight. Another portion of diphenylphosphoryl azide (2.26 g, 8.2 mmol) and 1,8-diazabicyclo[5.4.0]undec-7-ene (639 μL, 5 mmol) was added. The mixture was refluxed for additional 4 hours. The mixture was quenched with water (300 mL) and extracted with ethyl acetate (3×200 mL). The combined organic extracts were washed with brine, dried over sodium sulfate, and concentrated. The residue was purified by flash chromatography (80 g silica gel column, eluting with 0-10% methanol in dichloromethane) to give the title compound (2.8 g, 90%) as an oil. MS (ESI): 756 m/z [M+H]⁺, retention time: 2.03 minutes, purity: 82% (254 nm) (LC-MS method 2).

Mixture of Ethyl 3-[3-(24,30-difluoro-6,11,11-trimethyl-21-tetrahydropyran-2-yl-26-oxa-3,13,14,15,19,21,33-heptazahexacyclo[25.3.1.12,5.112,15.017,25.018,22]tritriaconta-1(31),2,4,12(32),13,17,19,22,24,27,29-undecaen-6-yl)phenyl]propanoate and 24,30-difluoro-6,11,11-trimethyl-6-phenyl-21-tetrahydropyran-2-yl-26-oxa-3,13,14,15,19,21,33-heptazahexacyclo[25.3.1.12,5.112,15.017,25.018,22]tritriaconta-1(31),2,4,12(32),13,17,19,22,24,27,29-undecaene

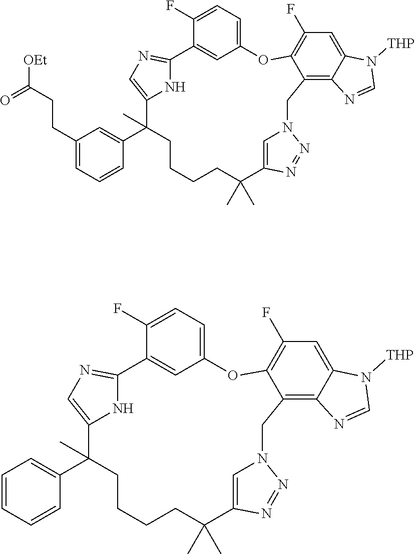

Step C: Exchanging 4-(azidomethyl)-6-fluoro-5-(4-fluoro-3-(5-(2-(3-iodophenyl)oct-7-yn-2-yl)-1H-imidazol-2-yl)phenoxy)-1H-indole (Step A product of Example 2) with 4-(azidomethyl)-5-(3-(5-(2-(3-bromophenyl)-7,7-dimethylnon-8-yn-2-yl)-1H-imidazol-2-yl)-4-fluorophenoxy)-6-fluoro-1-(tetrahydro-2H-pyran-2-yl)-1H-benzo[d]imidazole (Step B product of this example, 3 g, 3.81 mmol), the reaction procedure sequence (Steps B to D) described for Example 2 was used to prepare the title compound (504 mg) as an inseparable mixture. MS (ESI): 778 m/z [M+H]⁺, retention time: 1.85 minutes, purity: 86% (214 nm) (LC-MS method 2) (for compound with ethyl propionate ester tail).

Mixture of ethyl 3-[3-(24,30-Difluoro-6,11,11-trimethyl-26-oxa-3,13,14,15,19,21,33-heptazahexacyclo[25.3.1.12,5.112,15.017,25.018,22]tritriaconta-1(31),2,4,12(32),13,17,19,22,24,27,29-undecaen-6-yl)phenyl]propanoate and 24,30-Difluoro-6,11,11-trimethyl-6-phenyl-26-oxa-3,13,14,15,19,21,33-heptazahexacyclo[25.3.1.12,5.112,15.017,25.018,22]tritriaconta-1(31),2,4,12(32),13,17,19,22,24,27,29-undecaene

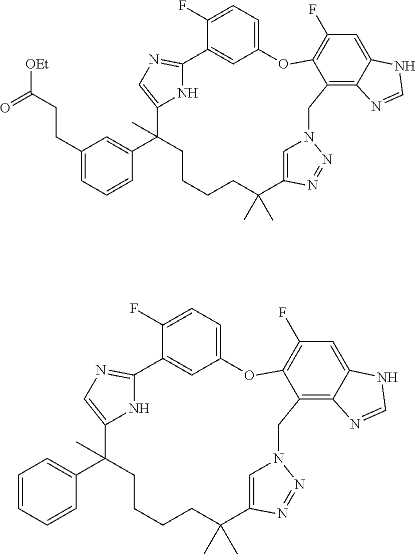 [SMILES: CCOC(=O)CCc1cccc(C2(C)CCCCC(C)(C)c3cn(nn3)Cc3c(c(F)cc4[nH]cnc34)Oc3ccc(F)c(c3)-c3ncc2[nH]3)c1 ; CC1(C)CCCCC(C)(c2ccccc2)c2cnc([nH]2)-c2cc(ccc2F)Oc2c(F)cc3[nH]cnc3c2Cn2cc1nn2]

Step D: To a stirred solution of Step C product (504 mg, 0.648 mmol) in ethanol (2 mL) was added 4 M hydrogen chloride in dioxane (10 mL). The mixture was stirred at 40° C. for four hours and concentrated. The residue was purified by automated flash chromatography (20 g silica gel column, eluting with 0-10% 7 N ammonia methanol solution in dichloromethane) to give the title compound (320 mg, 71%) as a mixture. MS (ESI): 694 m/z [M+H]⁺, retention time: 1.70 minutes, purity: 96% (214 nm) (LC-MS method 2) (for compound with ethyl propionate ester tail).

Compounds 67A, 67B: 3-[3-(24,30-Difluoro-6,11,11-trimethyl-26-oxa-3,13,14,15,19,21,33-heptazahexacyclo[25.3.1.12,5.112,15.017,25.018,22]tritriaconta-1(31),2,4,12(32),13,17,19,22,24,27,29-undecaen-6-yl)phenyl]propanoic acid

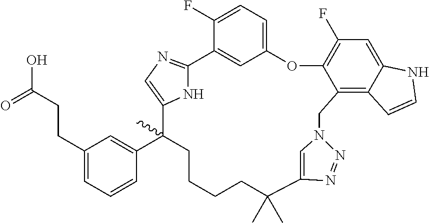 [SMILES: CC1(C)CCCCC(C)(c2cccc(CCC(=O)O)c2)c2cnc([nH]2)-c2cc(ccc2F)Oc2c(F)cc3[nH]ccc3c2Cn2cc1nn2]

Compound 68: 24,30-Difluoro-6,11,11-trimethyl-6-phenyl-26-oxa-3,13,14,15,19,21,33-heptazahexacyclo[25.3.1.12,5.112,15.017,25.018,22]tritriaconta-1(31),2,4,12(32),13,17,19,22,24,27,29-undecaene

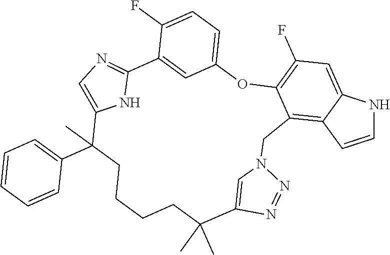 [SMILES: CC1(C)CCCCC(C)(c2ccccc2)c2cnc([nH]2)-c2cc(ccc2F)Oc2c(F)cc3[nH]ccc3c2Cn2cc1nn2]

Step E: The Step D product (650 mg, 0/937 mmol) was subject to chiral SFC separation using SFC-80 (Thar, Waters) under the following condition: Column: RRWHELK 20*250 mm, 10 μm; Column temperature: 35° C.; Mobile phase: carbon dioxide/ethanol (0.2% 7 M methanol ammonia as additive)=50/50; Flow rate: 80 g/minutes; Back pressure: 100 bar; Detection wavelength: 214 nm; Cycle time: 6.5 minutes; Sample solution: 0.65 g dissolved in 40 mL methanol; Injection volume: 2 mL. The first eluent (190 mg, 29%), Enantiomer 1, was further hydrolyzed to Compound 67A (170 mg, 94%) as a white solid; The second eluent (190 mg, 29%), Enantiomer 2, was further hydrolyzed to Compound 67B (156.6 mg, 86%, white solid). Both of these hydrolyses followed the conditions described for Step E of Example 2. The third eluent (9.1 mg, 1%), Compound 68, is the debromination product of the Heck reaction on corresponding Step C of Example 2.

Compound 67A: MS (ESI): 666 m/z [M+H]⁺, retention time: 1.37 minutes, purity: >99% (214 nm) (LC-MS method 2); Chiral HPLC purity: 96% ee; ¹H NMR (400 MHz, CD₃OD) δ 8.35 (s, 1H), 7.72 (s, 1H), 7.48 (d, J=10.1 Hz, 1H), 7.34-7.21 (m, 2H), 7.15-7.02 (m, 3H), 6.97 (d, J=7.6 Hz, 2H), 6.86 (s, 1H), 6.06 (d, J=14.1 Hz, 1H), 5.88 (d, J=14.3 Hz, 1H), 2.82 (t, J=7.6 Hz, 2H), 2.50 (t, J=7.7 Hz, 2H), 2.10-1.84 (m, 2H), 1.49-1.44 (m, 4H), 1.37-1.23 (m, 2H), 1.15 (s, 3H), 1.11 (s, 3H), 1.07-0.96 (m, 1H), 0.72 (brs, 1H), 0.51 (brs, 1H).

Compound 67B: MS (ESI): 666 m/z [M+H]⁺, retention time: 1.37 minutes, purity: >99% (214 nm) (LC-MS method 2); Chiral HPLC purity: 100% ee; ¹H NMR (400 MHz, CD₃OD) δ 8.35 (s, 1H), 7.72 (s, 1H), 7.48 (d, J=9.8 Hz, 1H), 7.36-7.24 (m, 2H), 7.12-7.06 (m, 3H), 6.99-6.94 (m, 2H), 6.85 (s, 1H), 6.06 (d, J=14.2 Hz, 1H), 5.88 (d, J=14.3 Hz, 1H), 2.82 (t, J=7.7 Hz, 2H), 2.50 (t, J=7.7 Hz, 2H), 2.12-1.85 (m, 2H), 1.49-1.44 (m, 4H), 1.39-1.23 (m, 2H), 1.15 (s, 3H), 1.11 (s, 3H), 1.01 (s, 1H), 0.73 (s, 1H), 0.50 (s, 1H).

Compound 68: MS (ESI): 594 m/z [M+H]⁺, retention time: 1.68 minutes, purity: 98% (254 nm) (LC-MS method 4); ¹H NMR (400 MHz, CD₃OD) δ 8.34 (s, 1H), 7.79 (s, 1H), 7.45 (s, 1H), 7.32-7.23 (m, 2H), 7.22-6.78 (m, 7H), 6.07 (d, J=16.3 Hz, 1H), 5.88 (d, J=15.5 Hz, 1H), 2.13-1.85 (m, 2H), 1.48-1.40 (m, 4H), 1.31 (s, 3H), 1.15 (s, 3H), 1.11 (s, 3H), 1.00-0.5 (m, 2H).

Example 69. Compound 69. 3-[3-(24-Fluoro-6,11,11-trimethyl-26-oxa-3-thia-13,14,15,21,33-pentazahexacyclo[25.3.1.12,5.112,15.017,25.018,22]tritriaconta-1(31),2(33),4,12(32),13,17,19,22,24,27,29-undecaen-6-yl)phenyl]propanoic acid

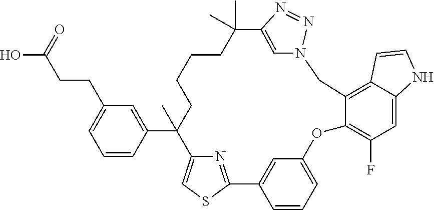 [SMILES: CC1(C)CCCCC(C)(c2cccc(CCC(=O)O)c2)c2csc(n2)-c2cccc(c2)Oc2c(F)cc3[nH]ccc3c2Cn2cc1nn2]

and

Example 70. Compound 70. 24-Fluoro-6,11,11-trimethyl-6-phenyl-26-oxa-3-thia-13,14,15,21,33-pentazahexacyclo[25.3.1.12,5.112,15.017,25.018,22]tritriaconta-1(31),2(33),4,12(32),13,17,19,22,24,27,29-undecaene

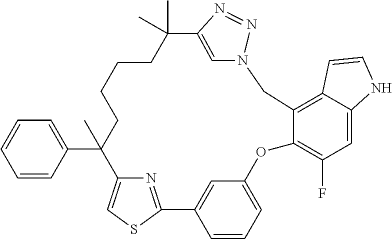 [SMILES: CC1(C)CCCCC(C)(c2ccccc2)c2csc(n2)-c2cccc(c2)Oc2c(F)cc3[nH]ccc3c2Cn2cc1nn2]

4-(2-(3-Bromophenyl)-7,7-dimethylnon-8-yn-2-yl)-2-(3-((6-fluoro-4-vinyl-1H-indol-5-yl)oxy)phenyl)thiazole

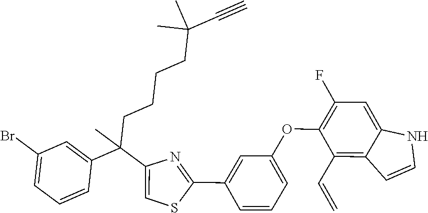 [SMILES: C#CC(C)(C)CCCCC(C)(c1cccc(Br)c1)c1csc(-c2cccc(Oc3c(F)cc4[nH]ccc4c3C=C)c2)n1]

Step A: To a stirred solution of 3-((6-fluoro-4-vinyl-1H-indol-5-yl)oxy)benzothioamide (Intermediate 56, 1.5 g, 4.80 mmol) in dimethylformamide (10 mL) was added 1-bromo-3-(3-bromophenyl)-3,8,8-trimethyldec-9-yn-2-one (Intermediate 2-39, 2.26 g, 5.28 mmol). The reaction was stirred at 80° C. overnight. The mixture was cooled to room temperature, diluted with water (50 mL), and extracted with ethyl acetate (3×50 mL). The combined organic extracts were washed with water, brine, dried over sodium sulfate, and concentrated. The residue was purified by automated flash chromatography (80 g silica gel column, eluting with 0-50% ethyl acetate in petroleum ether) to give the title compound (2 g, 59%) as a solid. MS (ESI): 641, 643 m/z [M+H]⁺, retention time: 1.96 minutes, purity: 91% (254 nm) (LC-MS method 15).

5-(3-(4-(2-(3-Bromophenyl)-7,7-dimethylnon-8-yn-2-yl)thiazol-2-yl)phenoxy)-6-fluoro-1H-indole-4-carbaldehyde

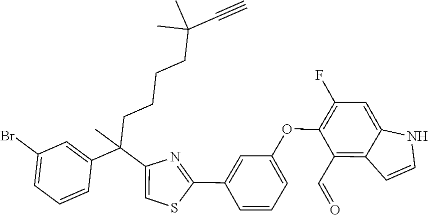 [SMILES: C#CC(C)(C)CCCCC(C)(c1cccc(Br)c1)c1csc(-c2cccc(Oc3c(F)cc4[nH]ccc4c3C=O)c2)n1]

Step B: To a stirred solution of Step A product (1.5 g, 2.34 mmol) in tetrahydrofuran (90 mL) and water (30 mL) was added 1 mg/mL osmium tetroxide aqueous solution (10 mL) and sodium periodate (1.5 g, 7.01 mmol). The reaction was stirred at room temperature overnight. The solution was diluted with water (200 mL) and extracted with ethyl acetate (3×100 mL). The combined organic phase was washed with brine, dried over sodium sulfate, and concentrated. The obtained crude title compound (1.53 g, 99%) was used for the next step without further purification. MS (ESI): 643, 645 m/z [M+H]⁺, retention time: 2.76 minutes, purity: 55% (214 nm) (LC-MS method 2).

(5-(3-(4-(2-(3-Bromophenyl)-7,7-dimethylnon-8-yn-2-yl)thiazol-2-yl)phenoxy)-6-fluoro-1H-indol-4-yl)methanol

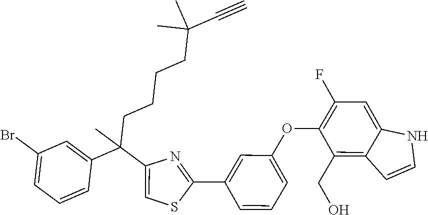 [SMILES: C#CC(C)(C)CCCCC(C)(c1cccc(Br)c1)c1csc(-c2cccc(Oc3c(F)cc4[nH]ccc4c3CO)c2)n1]

Step C: To a stirred solution of Step B product (1.53 g, 2.38 mmol) in ethanol (20 mL) was added sodium borohydride (135 mg, 3.57 mmol). The reaction mixture was stirred at room temperature for 1 hour. The solvent was removed. The residue was partitioned between water (50 mL) and ethyl acetate (50 mL). The separated organic layer, combined with two additional ethyl acetate extracts (2×50 mL), was washed with brine, dried over sodium sulfate, and concentrated. The crude product was purified by automated silica gel column (45 g column, eluting with 0-60% ethyl acetate in petroleum ether) to afford the title compound (900 mg, 55%) as a solid. MS (ESI): 645, 647 m/z [M+H]⁺, retention time: 2.54 minutes, purity: 93% (214 nm) (LC-MS method 2). 2-(3-((4-(Azidomethyl)-6-fluoro-1H-indol-5-yl)oxy)phenyl)-4-(2-(3-bromophenyl)-7,7-dimethylnon-8-yn-2-yl)thiazole

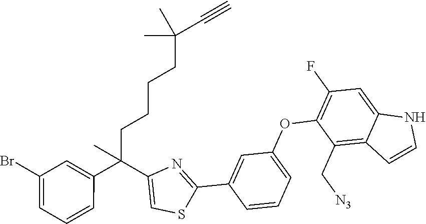 [SMILES: C#CC(C)(C)CCCCC(C)(c1cccc(Br)c1)c1csc(-c2cccc(Oc3c(F)cc4[nH]ccc4c3CN=[N+]=[N-])c2)n1]

Step D: To a stirred solution of Step C product (900 mg, 1.39 mmol) in tetrahydrofuran (20 mL) was added diphenylphosphoryl azide (767 mg, 2.79 mmol) and 1,8-Diazabicyclo[5.4.0]undec-7-ene (254 mg, 1.67 mmol). The reaction was refluxed overnight.

Another portion of diphenylphosphoryl azide (767 mg, 2.79 mmol) and 1,8-diazabicyclo[5.4.0]undec-7-ene (254 mg, 1.67 mmol) was added. The mixture was refluxed for an additional 6 hours and cooled to room temperature. The reaction was quenched with water (50 mL), extracted with ethyl acetate (3×30 mL). The combined organic extracts were washed with brine, dried over sodium sulfate, and concentrated. The residue was purified by automated flash chromatography (25 g silica gel column, eluted with 0-60% ethyl acetate in petroleum ether) to afford the title compound (440 mg, 47%). MS (ESI): 670, 672 m/z [M+H]⁺, retention time: 2.81 minutes, purity: >99% (214 nm) (LC-MS method 9).

Compound 69: 3-[3-(24-Fluoro-6,11,11-trimethyl-26-oxa-3-thia-13,14,15,21,33-pentazahexacyclo[25.3.1.12,5.112,15.017,25.018,22]tritriaconta-1(31),2(33),4,12(32),13,17,19,22,24,27,29-undecaen-6-yl)phenyl]propanoic acid

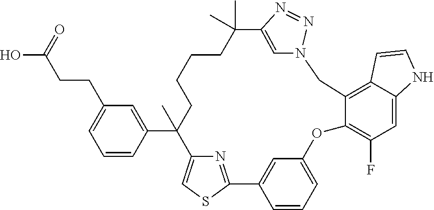 [SMILES: CC1(C)CCCCC(C)(c2cccc(CCC(=O)O)c2)c2csc(n2)-c2cccc(c2)Oc2c(F)cc3[nH]ccc3c2Cn2cc1nn2]

Compound 70. 24-Fluoro-6,11,11-trimethyl-6-phenyl-26-oxa-3-thia-13,14,15,21,33-pentazahexacyclo[25.3.1.12,5.112,15.017,25.018,22]tritriaconta-1(31),2(33),4,12(32),13,17,19,22,24,27,29-undecaene

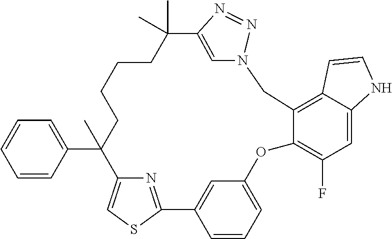 [SMILES: CC1(C)CCCCC(C)(c2ccccc2)c2csc(n2)-c2cccc(c2)Oc2c(F)cc3[nH]ccc3c2Cn2cc1nn2]

Exchanging 4-(azidomethyl)-6-fluoro-5-(4-fluoro-3-(5-(2-(3-iodophenyl)oct-7-yn-2-yl)-1H-imidazol-2-yl)phenoxy)-1H-indole (Step A product of Example 2) with 2-(3-((4-(azidomethyl)-6-fluoro-1H-indol-5-yl)oxy)phenyl)-4-(2-(3-bromophenyl)-7,7-dimethylnon-8-yn-2-yl)thiazole (Step D product of this example, 440 mg, 0.66 mmol), the reaction procedure sequence (Steps B to E) described in Example 2 was used to prepare the title compounds 69 (35.5 mg, the second eluent) and Compound 70 (14 mg, the first eluent). Compound 70 is a debromination product of the Heck reaction, the corresponding Step C of Example 2. The two compounds were separated by preparative HPLC at last step, corresponding Step E.

Compound 69: MS (ESI): 664 m/z [M+H]⁺, retention time: 2.36 minutes, purity: >99% (214 nm) (LC-MS method 2). ¹H NMR (400 MHz, CD₃OD) δ 7.54-7.45 (m, 2H), 7.41-7.35 (m, 2H), 7.34-7.30 (m, 1H), 7.27 (s, 1H), 7.26-7.23 (m, 1H), 7.22 (s, 1H), 7.14-7.09 (m, 2H), 7.02-6.98 (m, 2H), 6.69 (d, J=3.2 Hz, 1H), 5.98 (d, J=14.5 Hz, 1H), 5.81 (d, J=14.5 Hz, 1H), 2.85 (t, J=7.8 Hz, 2H), 2.53 (t, J=7.8 Hz, 2H), 2.20-2.11 (m, 1H), 1.87-1.77 (m, 1H), 1.59 (s, 3H), 1.37-1.29 (m, 3H), 1.16 (s, 3H), 1.04 (s, 3H), 0.93-0.89 (m, 1H), 0.75-0.69 (m, 1H), 0.57-0.49 (m, 1H).

Compound 70: MS (ESI): 592 m/z [M+H]⁺, retention time: 2.56 minutes, purity: >99% (214 nm) (LC-MS method 2). ¹H NMR (400 MHz, CD₃OD) δ 7.50-7.41 (m, 2H), 7.35 (d, J=3.2 Hz, 1H), 7.32-7.23 (m, 3H), 7.22 (s, 1H), 7.19-7.15 (m, 5H), 7.12-7.07 (m, 1H), 6.65 (d, J=3.2 Hz, 1H), 5.93 (d, J=14.5 Hz, 1H), 5.78 (d, J=14.5 Hz, 1H), 2.20-2.09 (m, 1H), 1.91-1.80 (m, 1H), 1.58 (s, 3H), 1.38-1.27 (m, 3H), 1.15 (s, 3H), 1.02 (s, 3H), 0.96-0.86 (m, 1H), 0.80-0.72 (m, 1H), 0.62-0.53 (m, 1H).

Example 71. Compound 70. 3-[3-(24,30-Difluoro-11,11-dimethyl-10,26-dioxa-3,5,13,14,15,21,33-heptazahexacyclo[25.3.1.12,5.112,15.017,25.018,22]tritriaconta-1(31),2(33),3,12(32),13,17,19,22,24,27,29-undecaen-6-yl)phenyl]propanoic acid

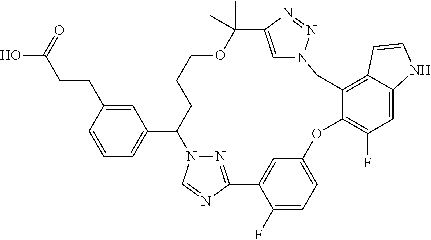 [SMILES: CC1(C)OCCCC(c2cccc(CCC(=O)O)c2)n2cnc(n2)-c2cc(ccc2F)Oc2c(F)cc3[nH]ccc3c2Cn2cc1nn2]

(5-(3-(1-(1-(3-Bromophenyl)-4-((2-methylbut-3-yn-2-yl)oxy)butyl)-1H-1,2,4-triazol-3-yl)-4-fluorophenoxy)-6-fluoro-1-tosyl-1H-indol-4-yl)methanol

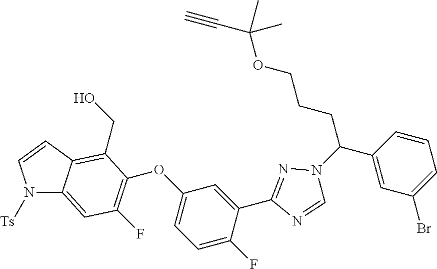 [SMILES: C#CC(C)(C)OCCCC(c1cccc(Br)c1)n1cnc(-c2cc(Oc3c(F)cc4c(ccn4S(=O)(=O)c4ccc(C)cc4)c3CO)ccc2F)n1]

Step A: To a stirred solution of (6-fluoro-5-(4-fluoro-3-(1H-1,2,4-triazol-3-yl)phenoxy)-1-tosyl-1H-indol-4-yl)methanol (Intermediate 58, 1.2 g, 2.42 mmol) in dimethylformamide (15 mL) was added 1-bromo-3-(1-bromo-4-((2-methylbut-3-yn-2-yl)oxy)butyl)benzene (Intermediate 59, 904 mg, 2.42 mmol) and cesium carbonate (1.58 g, 4.83 mmol). The reaction mixture was stirred at room temperature for 3 hours. Then 15 mL of water was added. The mixture was extracted with ethyl acetate (3×50 mL). The combined organic layers were dried over sodium sulfate, and concentrated. The residue was purified by silica gel (petroleum ether: ethyl acetate=1:1) to give the title compound (760 mg, 39%) as a yellow solid. MS (ESI): 789 m/z [M+H]⁺, retention time: 2.47 minutes, purity: 97% (214 nm) (LC-MS method 2).

(5-(3-(1-(1-(3-Bromophenyl)-4-((2-methylbut-3-yn-2-yl)oxy)butyl)-1H-1,2,4-triazol-3-yl)-4-fluorophenoxy)-6-fluoro-1H-indol-4-yl)methanol

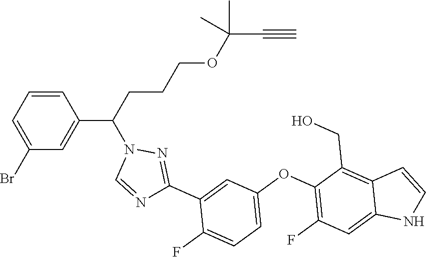 [SMILES: C#CC(C)(C)OCCCC(c1cccc(Br)c1)n1cnc(-c2cc(Oc3c(F)cc4[nH]ccc4c3CO)ccc2F)n1]

Step B: To a stirred solution of Step A product (760 mg, 0.96 mmol) in methanol (14.3 mL) was added potassium carbonate (266 mg, 1.92 mmol). The reaction mixture was heated at 60° C. for 2 hours and concentrated. The residue was purified by silica gel column (eluting with petroleum ether: ethyl acetate=1:1) to give the title compound (530 mg, 72%) as a yellow solid. MS (ESI): 635 m/z [M+H]⁺, retention time: 2.22 minutes, purity: 83% (214 nm) (LC-MS method 2). 4-(Azidomethyl)-5-(3-(1-(1-(3-bromophenyl)-4-((2-methylbut-3-yn-2-yl)oxy)butyl)-1H-1,2,4-triazol-3-yl)-4-fluorophenoxy)-6-fluoro-1H-indole

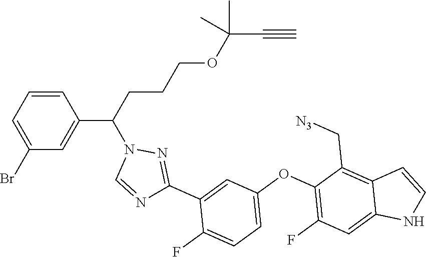 [SMILES: C#CC(C)(C)OCCCC(c1cccc(Br)c1)n1cnc(-c2cc(Oc3c(F)cc4[nH]ccc4c3CN=[N+]=[N-])ccc2F)n1]

Step C: To a stirred solution of Step B product (530 mg, 0.83 mmol) in tetrahydrofuran (26.5 mL) was added diphenyl phosphoryl azide (459 mg, 1.67 mmol) and 1,8-Diazabicyclo[5.4.0]undec-7-ene (152 mg, 1.0 mmol). The mixture was vigorously stirred at 70° C. for 18 hours and concentrated. The residue was purified by silica gel column eluting with methanol/dichloromethane=1/20 to give the title compound as a solid (410 mg, 73%). MS (ESI): 660 m/z [M+H]⁺, retention time: 2.38 minutes, purity: 98% (214 nm) (LC-MS method 2).

Compound 70: 3-[3-(24,30-Difluoro-11,11-dimethyl-10,26-dioxa-3,5,13,14,15,21,33-heptazahexacyclo[25.3.1.12,5.112,15.017,25.018,22]tritriaconta-1(31),2(33),3,12(32),13,17,19,22,24,27,29-undecaen-6-yl)phenyl]propanoic acid

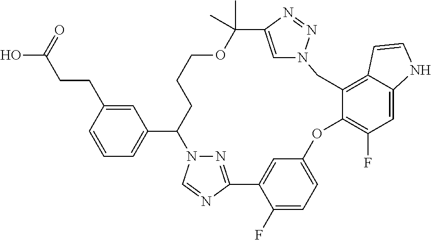 [SMILES: CC1(C)OCCCC(c2cccc(CCC(=O)O)c2)n2cnc(n2)-c2cc(ccc2F)Oc2c(F)cc3[nH]ccc3c2Cn2cc1nn2]

Step D: Exchanging 4-(azidomethyl)-6-fluoro-5-(4-fluoro-3-(5-(2-(3-iodophenyl)oct-7-yn-2-yl)-1H-imidazol-2-yl)phenoxy)-1H-indole (Step A product of Example 2) with 4-(azidomethyl)-5-(3-(1-(1-(3-bromophenyl)-4-((2-methylbut-3-yn-2-yl)oxy)butyl)-1H-1,2,4-triazol-3-yl)-4-fluorophenoxy)-6-fluoro-1H-indole (Step C product of this example, 410 mg, 0.13 mmol), the reaction procedure sequence (Steps B to E) described in Example 2 was used to prepare the title compound (18 mg).

Compound 70: MS (ESI): 654 m/z [M+H]⁺, retention time: 1.97 minutes, purity: 97% (214 nm) (LC-MS method 2). ¹H NMR (400 MHz, CD₃OD) δ 8.50 (s, 1H), 7.47-7.08 (m, 10H), 6.16 (d, J=3.2 Hz, 1H), 6.01-5.89 (m, 2H), 5.49-5.40 (m, 1H), 3.15-3.08 (m, 1H), 2.89-2.80 (m, 2H), 2.68-2.60 (m, 1H), 2.58-2.50 (m, 2H), 1.85-1.75 (m, 1H), 1.65-1.55 (m, 1H), 1.45 (s, 3H), 1.39-1.30 (m, 4H), 1.17-1.05 (m, 1H).

Example 72. Compound 72. 3-[3-(24,30-Difluoro-6,11,11-trimethyl-14,26-dioxa-3,13,21,32,33-pentazahexacyclo[25.3.1.12,5.112,15.017,25.018,22]tritriaconta-1(31),2,4,12,15(32),17,19,22,24,27,29-undecaen-6-yl)phenyl]propanoic acid

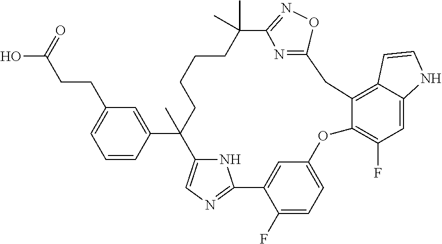 [SMILES: CC1(C)CCCCC(C)(c2cccc(CCC(=O)O)c2)c2cnc([nH]2)-c2cc(ccc2F)Oc2c(F)cc3[nH]ccc3c2Cc2nc1no2]

Ethyl 2-(5-(3-(5-(7-cyano-2-(3-iodophenyl)-7-methyloctan-2-yl)-1H-imidazol-2-yl)-4-fluorophenoxy)-6-fluoro-1H-indol-4-yl)acetate

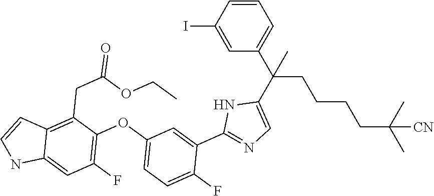 [SMILES: CCOC(=O)Cc1c(Oc2ccc(F)c(-c3ncc(C(C)(CCCCC(C)(C)C#N)c4cccc(I)c4)[nH]3)c2)c(F)cc2[nH]ccc12]

Step A: To a stirred solution of ethyl 2-(5-(3-carbamimidoyl-4-fluorophenoxy)-6-fluoro-1H-indol-4-yl)acetate (Intermediate 29, 1.5 g, 4.02 mmol) in dry dimethylformamide (28 mL) in a glove box was added 9-bromo-7-(3-iodophenyl)-2,2,7-trimethyl-8-oxononanenitrile (Intermediate 2-41, 1.798 g, 4.0 mmol) and sodium bicarbonate (675 mg, 8.04 mmol). The mixture was stirred at 80° C. overnight and cooled to room temperature, quenched with water (100 mL), and extracted with ethyl acetate (100 mL). The separated organic layer, combined with two additional ethyl acetate extracts, was washed with brine, dried over sodium sulfate, and concentrated. The residue was purified by silica gel column chromatography (40 g silica gel column, eluting with 0-50% ethyl acetate in petroleum ether) to give the title compound (2.1 g, 70%) as yellow solid. MS (ESI): 751 m/z [M+H]⁺, retention time: 1.95 minutes, purity: 93% (214 nm) (LC-MS method 2).

2-(5-(3-(5-(7-Cyano-2-(3-iodophenyl)-7-methyloctan-2-yl)-1H-imidazol-2-yl)-4-fluorophenoxy)-6-fluoro-1H-indol-4-yl)acetic acid

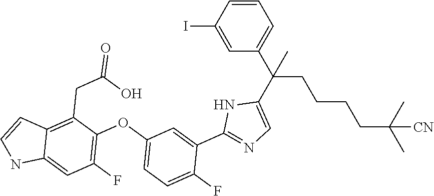 [SMILES: CC(C)(C#N)CCCCC(C)(c1cccc(I)c1)c1cnc(-c2cc(Oc3c(F)cc4[nH]ccc4c3CC(=O)O)ccc2F)[nH]1]

Step B: To a stirred solution of Step A product (2.1 g, 2.8 mmol) in tetrahydrofuran (98.1 mL) and methanol (32.7 mL) was added lithium hydroxide (32.7 mL, 1 M in water, 32.7 mmol). The mixture was stirred at room temperature overnight, then quenched with 1N hydrochloric acid until the pH was ˜5. The mixture was diluted with ethyl acetate (500 mL), washed with water and brine, dried over sodium sulfate and concentrated. The residue was purified by automated silica gel column chromatography (40 g silica gel column, eluting with 0-10% methanol in dichloromethane) to give the title compound (2.0 g, 98%) as white solid. MS (ESI): 723 m/z [M+H]⁺, retention time: 1.25 minutes, purity: 94% (254 nm) (LC-MS method 15).

(Z)-2-(5-(3-(5-(8-Amino-8-(hydroxyimino)-2-(3-iodophenyl)-7,7-dimethyloctan-2-yl)-1H-imidazol-2-yl)-4-fluorophenoxy)-6-fluoro-1H-indol-4-yl)acetic acid

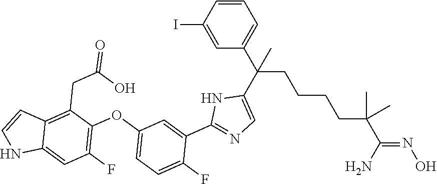 [SMILES: CC(C)(CCCCC(C)(c1cccc(I)c1)c1cnc(-c2cc(Oc3c(F)cc4[nH]ccc4c3CC(=O)O)ccc2F)[nH]1)/C(N)=N/O]

Step C: To a stirred solution of Step B product (1.1 g, 1.52 mmol) in dry ethanol (16 mL) was added hydroxylamine hydrochloride (2.11 g, 30.4 mmol) and triethylamine (4.24 mL, 30.4 mmol). The solution was heated at 80° C. in a sealed tube overnight, The mixture was diluted with ethyl acetate (200 mL), washed with water, brine, dried over sodium sulfate, and concentrated to give the crude title compound (1.15 g, 100%) as a white solid. The crude product was used for next step without further purification. MS (ESI): 756 m/z [M+H]⁺, retention time: 1.62 minutes, purity: 64% (254 nm) (LC-MS method 2).

24,30-difluoro-6-(3-iodophenyl)-6,11,11-trimethyl-14,26-dioxa-3,13,21,32,33-pentazahexacyclo[25.3.1.12,5.112,15.017,25.018,22]tritriaconta-1(30),2,4,12,15(32),17(25),18(22),19,23,27(31),28-undecaene

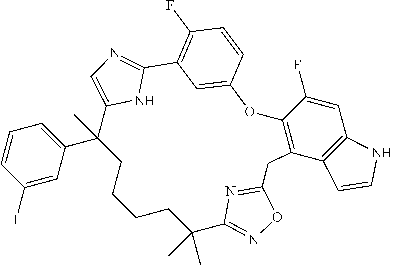 [SMILES: CC1(C)CCCCC(C)(c2cccc(I)c2)c2cnc([nH]2)-c2cc(ccc2F)Oc2c(F)cc3[nH]ccc3c2Cc2nc1no2]

Step D: To a stirred solution of Step C product (1.15 g, 1.52 mmol) in N-methyl-2-pyrrolidone (35 mL) was added 1-ethyl-3-(3-dimethylaminopropyl)carbodiimide hydrochloride (438 mg, 2.28 mmol) and hydroxy-benzotriazole (308 mg, 2.28 mmol). The solution was stirred at room temperature for 16 hours, then heated to 80° C. for 12 hours. The mixture was diluted with ethyl acetate (200 mL), washed with water, brine, dried over sodium sulfate, and concentrated. The residue was purified by silica gel column chromatography (20 g silica gel column, eluting with 0-100% ethyl acetate in petroleum ether) to give the title compound (180 mg, 16% two steps) as a yellow solid. MS (ESI): 720 m/z [M+H]⁺, retention time: 1.96 minutes, purity: 96% (254 nm) (LC-MS method 2).

Compound 72: 3-[3-(24,30-Difluoro-6,11,11-trimethyl-14,26-dioxa-3,13,21,32,33-pentazahexacyclo[25.3.1.12,5.112,15.017,25.018,22]tritriaconta-1(31),2,4,12,15(32),17,19,22,24,27,29-undecaen-6-yl)phenyl]propanoic acid

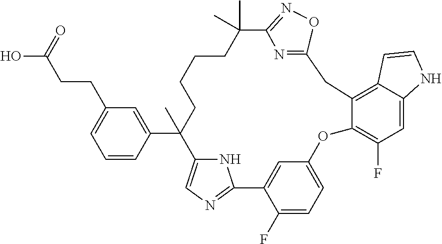 [SMILES: CC1(C)CCCCC(C)(c2cccc(CCC(=O)O)c2)c2cnc([nH]2)-c2cc(ccc2F)Oc2c(F)cc3[nH]ccc3c2Cc2nc1no2]

Exchanging 23,29-difluoro-6-(3-iodophenyl)-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaene (Step B product of Example 2) with 24,30-difluoro-6-(3-iodophenyl)-6,11,11-trimethyl-14,26-dioxa-3,13,21,32,33-pentazahexacyclo[25.3.1.12,5.112,15.017,25.018,22]tritriaconta-1(30),2,4,12,15(32),17(25),18(22),19,23,27(31),28-undecaene (Step D product of this example, 180 mg, 0.25 mmol), the reaction sequence (Steps C to E) described for Example 2 was used to prepare the title compound (33 mg, white solid). MS (ESI): 666 m/z [M+H]⁺, retention time: 1.49 minutes, purity: 98% (214 nm) (LC-MS method 16). ¹H NMR (400 MHz, CD₃OD) δ 7.32 (m, 2H), 7.22-7.09 (m, 4H), 7.10-6.98 (m, 3H), 6.95 (s, 1H), 6.55 (s, 1H), 4.43 (s, 2H), 2.85 (t, J=7.6 Hz, 2H), 2.51 (t, J=7.8 Hz, 2H), 2.25-2.02 (m, 2H), 1.71-1.68 (m, 1H), 1.54 (s, 3H), 1.35-1.29 (m, 2H), 1.24 (s, 3H), 1.18 (s, 3H), 1.06-1.00 (m, 1H), 0.95-0.88 (m, 1H), 0.81-0.76 (m, 1H).

Example 73. Compound 73A and Compound 73B. Enantiomers 1 and 2 of 2-[3-(24,30-Difluoro-6,11,11-trimethyl-26-oxa-3,13,14,15,21,33-hexazahexacyclo[25.3.1.12,5.112,15.017,25.018,22]-tritriaconta-1(31),2,4,12(32),13,17,19,22,24,27,29-undecaen-6-yl)phenyl]acetic acid

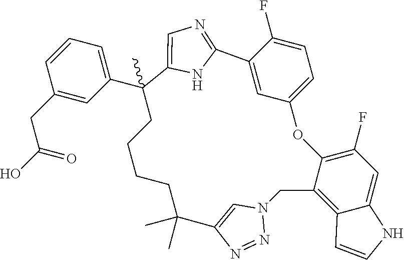 [SMILES: CC1(C)CCCCC(C)(c2cccc(CC(=O)O)c2)c2cnc([nH]2)-c2cc(ccc2F)Oc2c(F)cc3[nH]ccc3c2Cn2cc1nn2]

Exchanging 1-bromo-3-(3-iodophenyl)-3-methylnon-8-yn-2-one (Intermediate 2-51) with 1-bromo-8,8-difluoro-3-(3-iodophenyl)-3-methyldec-9-yn-2-one (Intermediate 2-42, 0.6 g, 1.42 mmol), the reaction procedure sequence (Steps A, B and E) described for Example 2 was used to prepare title compounds. The racemic methyl ester from corresponding Step B of Example 2 (0.28 g, 0.4 mmol) was subject to chiral SFC purification using SFC-80 (Thar, Waters) under the following condition: Column: RRWHELK 20*250 mm, 10 μm; Column temperature: 35° C., Mobile phase: carbon dioxide/ethanol (0.2% ammonia (7 M Methanol Ammonia) as additive)=50/50; Flow rate: 80 g/minutes; Back pressure: 100 bar; Detection wavelength: 214 nm; Cycle time: 6.5 minutes; Sample solution: 0.28 g dissolved in 40 mL methanol; Injection volume: 2 mL. The first eluent (100 mg, 36%, 100% ee), Enantiomer 1, was further hydrolyzed to Compound 73A (61.2 mg, 63%); The second eluent (120 mg, 43%, 95% ee), enantiomer 2, was further hydrolyzed to Compound 73B (108 mg, 92%), following the conditions described in step E of Example 2.

Compound 73A: MS (ESI): 651 m/z [M+H]⁺, retention time: 1.70 minutes, purity: >99% (254 nm) (LC-MS method 2). ¹H NMR (400 MHz, CD₃OD): δ 7.38-7.33 (m, 2H), 7.33-7.25 (m, 3H), 7.23-7.12 (m, 2H), 7.10 (s, 1H), 7.06 (d, J=7.6 Hz, 3H), 6.67 (d, J=3.1 Hz, 1H), 5.96 (d, J=14.4 Hz, 1H), 5.77 (d, J=14.4 Hz, 1H), 3.50 (s, 2H), 2.14-1.97 (m, 2H), 1.48 (s, 3H), 1.47-1.40 (m, 2H), 1.31-1.19 (m, 1H), 1.15 (s, 3H), 1.06 (s, 3H), 1.03-0.95 (m, 1H), 0.8-0.70 (m, 1H), 0.65-0.60 (m, 1H).

Compound 73B: MS (ESI): 651 m/z [M+H]⁺, retention time: 1.70 minutes, purity: >99% (254 nm) (LC-MS method 2). ¹H NMR (400 MHz, CD₃OD): δ 7.38-7.33 (m, 2H), 7.33-7.24 (m, 3H), 7.23-7.12 (m, 2H), 7.10 (s, 1H), 7.06 (d, J=7.6 Hz, 3H), 6.67 (d, J=3.1 Hz, 1H), 5.96 (d, J=14.4 Hz, 1H), 5.77 (d, J=14.4 Hz, 1H), 3.50 (s, 2H), 2.14-1.97 (m, 2H), 1.48 (s, 3H), 1.47-1.40 (m, 2H), 1.31-1.19 (m, 1H), 1.15 (s, 3H), 1.06 (s, 3H), 1.03-0.95 (m, 1H), 0.8-0.70 (m, 1H), 0.65-0.60 (m, 1H).

Example 74. Compound 74. 3-[3-(25,31-Difluoro-6-methyl-27,33-dioxa-3,14,22,34-tetrazaheptacyclo[26.3.1.12,5.113,16.010,12.018,26.019,23]tetratriaconta-1(32),2,4,13,15,18,20,23,25,28,30-undecaen-6-yl)phenyl]propanoic acid

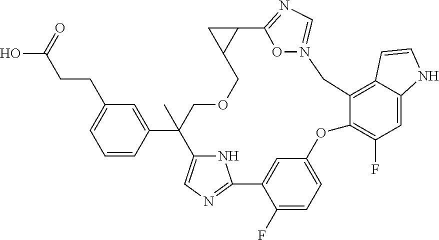

Exchanging 9-bromo-7-(3-iodophenyl)-7-methyl-8-oxononanenitrile (Intermediate 2-32) with ethyl 2-(6-bromo-4-(3-bromophenyl)-4-methyl-5-oxohexyl)cyclopropane-1-carboxylate (Intermediate 2-54, 1.8 g, 3.88 mmol), the reaction procedure sequence (Steps A to C) described for Example 52 was used to prepare the title compound (2 mg) as a diastereomeric mixture. MS (ESI): 649.1 m/z [M+H]⁺, retention time: 1.44 minutes, purity: 95% (214 nm) (LC-MS method 18).

Example 75. Compound 75. 3-[3-(24,30-Difluoro-6-methyl-13,26,32-trioxa-3,9,21,33-tetrazahexacyclo[25.3.1.12,5.17,10.017,25.018,22]tritriaconta-1(31),2,4,7,9,17,19,22,24,27,29-undecaen-6-yl)phenyl]propanoic acid

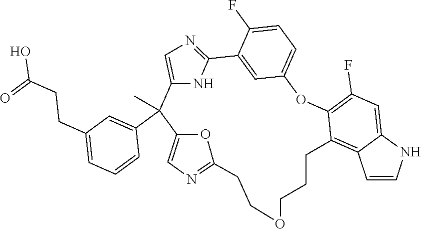 [SMILES: CC1(c2cccc(CCC(=O)O)c2)C2=CN=C(CCOCCCc3c(c(F)cc4[nH]ccc34)Oc3ccc(F)c(c3)-c3ncc1[nH]3)O2]

Tert-Butyl 3-(3-(5-(3-(5-(1-acetoxy-2-(3-bromophenyl)propan-2-yl)-1H-imidazol-2-yl)-4-fluorophenoxy)-6-fluoro-1H-indol-4-yl)propoxy)propanoate

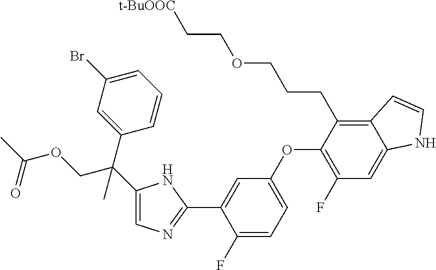

Step A: To a stirred solution of tert-butyl 3-(3-(5-(3-carbamimidoyl-4-fluorophenoxy)-6-fluoro-1H-indol-4-yl)propoxy)propanoate (Intermediate 29-1, 4.82 g, 10.2 mmol) and 4-bromo-2-(3-bromophenyl)-2-methyl-3-oxobutyl acetate (Intermediate 2-43, 3.5 g, 9.26 mmol) in dry dimethylformamide (120 mL) was added sodium bicarbonate (1.56 g, 18.5 mmol). The mixture was stirred at 90° C. for 8 hours. The mixture was cooled to room temperature, quenched with water (300 mL), and extracted with ethyl acetate (3×100 mL). The combined extracts were washed with brine, dried over sodium sulfate, and concentrated. The residue was purified by flash chromatography (120 g silica gel column, eluting with 0-60% ethyl acetate in petroleum ether) to give the title compound (4.5 g, 65%) as a solid. MS (ESI): 752, 754 m/z [M+H]⁺, retention time: 1.40 minutes, purity: 91% (254 nm) (LC-MS method 2).1H NMR (400 MHz, CD₃OD) δ 7.47 (s, 2H), 7.38-3.27 (m, 3H), 7.25-7.09 (m, 3H), 7.02 (s, 1H), 6.82 (s, 1H), 6.56 (d, J=3.1 Hz, 1H), 4.57 (s, 2H), 3.58 (t, J=6.1 Hz, 2H), 3.42 (t, J=6.3 Hz, 2H), 2.97-2.84 (m, 2H), 2.41 (t, J=6.1 Hz, 2H), 1.95 (s, 3H), 1.91-1.81 (m, 2H), 1.75 (s, 3H), 1.41 (s, 9H).

3-(3-(5-(3-(5-(2-(3-Bromophenyl)-1-hydroxypropan-2-yl)-1H-imidazol-2-yl)-4-fluorophenoxy)-6-fluoro-1H-indol-4-yl)propoxy)propanoic acid

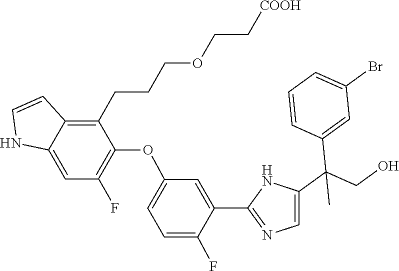 [SMILES: CC(CO)(c1cccc(Br)c1)c1cnc(-c2cc(Oc3c(F)cc4[nH]ccc4c3CCCOCCC(=O)O)ccc2F)[nH]1]

Step B: To a stirred solution of Step A product (4.5 g, 5.98 mmol) in tetrahydrofuran (270 mL) and methanol (90 mL) was added 1 M lithium hydroxide (90 mL). The mixture was stirred at room temperature over the weekend and concentrated. The aqueous residue was acidified with 1N hydrochloric acid to pH ˜4 and extracted with ethyl acetate (4×50 mL). The combined organic extracts were washed with brine, dried over sodium sulfate, and concentrated to give the title compound (3.9 g, 99%) as a solid. MS (ESI): 654, 656 m/z [M+H]⁺, retention time: 1.12 minutes, purity: 94% (254 nm) (LC-MS method 2). ¹H NMR (400 MHz, CD₃OD) δ 7.52-7.45 (m, 2H), 7.36 (d, J=7.8 Hz, 1H), 7.30-7.25 (m, 2H), 7.21 (d, J=7.8 Hz, 1H), 7.19-7.10 (m, 2H), 7.05 (s, 1H), 6.86-6.76 (m, 1H), 6.56 (d, J=3.2 Hz, 1H), 4.10-3.92 (m, 2H), 3.61 (t, J=6.2 Hz, 2H), 3.42 (t, J=6.2 Hz, 2H), 2.91 (t, J=7.6 Hz, 2H), 2.48 (t, J=6.2 Hz, 2H), 1.93-1.81 (m, 2H), 1.68 (s, 3H).

(E)-3-(3-(5-(3-(5-(2-(3-(3-Ethoxy-3-oxoprop-1-en-1-yl)phenyl)-1-hydroxypropan-2-yl)-1H-imidazol-2-yl)-4-fluorophenoxy)-6-fluoro-1H-indol-4-yl)propoxy)propanoic acid

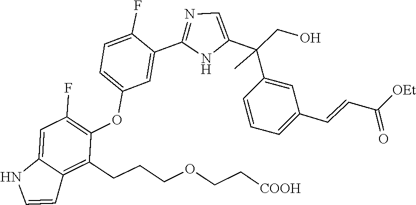 [SMILES: CCOC(=O)/C=C/c1cccc(C(C)(CO)c2cnc(-c3cc(Oc4c(F)cc5[nH]ccc5c4CCCOCCC(=O)O)ccc3F)[nH]2)c1]

Step C: To a stirred and deassed solution of Step B product (3.9 g, 5.96 mmol) in dimethylformamide (80 mL) was added palladium (II) acetate (201 mg, 0.894 mmol), Tris(o-tolyl)phosphine (544 mg, 1.79 mmol), triethylamine (4.15 mL, 29.8 mmol) and ethyl acrylate (1.94 mL, 17.9 mmol). The reaction was stirred at 125° C. overnight, cooled to room temperature. The mixture was diluted with water (100 mL) and acidified by 2 N hydrochloric acid to pH ˜3 and extracted with ethyl acetate (4×100 mL). The combined organic extracts were washed with brine, dried over sodium sulfate, and concentrated. The residue was purified by automated flash chromatography (120 g silica gel column, eluting with 0-10% methanol in dichloromethane) to give the title compound (2.62 g, 65%) as a solid. MS (ESI): 674 m/z [M+H]⁺, retention time: 1.12 minutes, purity: 91% (214 nm) (LC-MS method 15).

3-(3-(5-(3-(5-(2-(3-(3-Ethoxy-3-oxopropyl)phenyl)-1-hydroxypropan-2-yl)-1H-imidazol-2-yl)-4-fluorophenoxy)-6-fluoro-1H-indol-4-yl)propoxy)propanoic acid

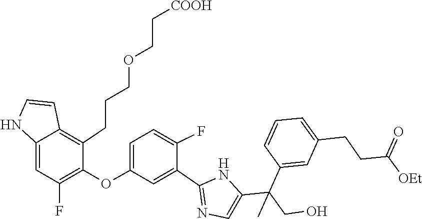 [SMILES: CCOC(=O)CCc1cccc(C(C)(CO)c2cnc(-c3cc(Oc4c(F)cc5[nH]ccc5c4CCCOCCC(=O)O)ccc3F)[nH]2)c1]

Step D: To a stirred solution of Step C product (2.62 g, 3.89 mmol) in ethanol (100 mL) was added Pd—C (10%, 1.31 g). The mixture was stirred at room temperature for 3 hours under a hydrogen balloon. The mixture was filtered through a pad of Celite, and the filter cake was rinsed with ethanol (2×50 mL). The filtrate was concentrated to give the title compound (2.38 g, 90%) as a solid. MS (ESI): 676 m/z [M+H]⁺, retention time: 1.10 minutes, purity: 91% (214 nm) (LC-MS method 15). ¹H NMR (400 MHz, CD₃OD) δ 7.49 (dd, J=6.1, 3.2 Hz, 1H), 7.28 (t, J=5.5 Hz, 1H), 7.22 (t, J=8.0 Hz, 1H), 7.18-7.10 (m, 4H), 7.07 (d, J=7.7 Hz, 1H), 7.00 (s, 1H), 6.86-6.78 (m, 1H), 6.56 (d, J=3.1 Hz, 1H), 4.14 (d, J=10.8 Hz, 1H), 4.05 (q, J=7.1 Hz, 2H), 3.91 (d, J=11.0 Hz, 1H), 3.66-3.56 (m, 2H), 3.43 (t, J=6.2 Hz, 2H), 2.90 (dt, J=11.7, 7.4 Hz, 4H), 2.58 (t, J=7.5 Hz, 2H), 2.47 (t, J=6.2 Hz, 2H), 1.92-1.81 (m, 2H), 1.68 (s, 3H), 1.18 (t, J=7.2 Hz, 3H).

Benzyl 3-(3-(5-(3-(5-(2-(3-(3-ethoxy-3-oxopropyl)phenyl)-1-hydroxypropan-2-yl)-1H-imidazol-2-yl)-4-fluorophenoxy)-6-fluoro-1H-indol-4-yl)propoxy)propanoate

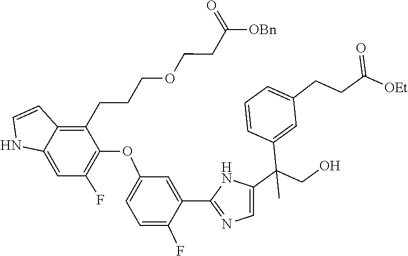 [SMILES: CCOC(=O)CCc1cccc(C(C)(CO)c2cnc(-c3cc(Oc4c(F)cc5[nH]ccc5c4CCCOCCC(=O)OCc4ccccc4)ccc3F)[nH]2)c1]

Step E: To a stirred solution of Step D product (2.18 g, 3.23 mmol) in tetrahydrofuran (80 mL) was added benzyl alcohol (1.22 g, 11.3 mmol),1-hydroxybenzotriazole (0.654 g, 4.84 mmol),1-ethyl-3-(3-dimethylaminopropyl)carbodiimide hydrochloride (0.928 g, 4.84 mmol). The mixture was stirred at room temperature for 16 hours, then diluted with ethyl acetate (50 mL). The solution was washed with saturated sodium bicarbonate, brine, dried over sodium sulfate, and concentrated. The residue was purified by flash chromatography (40 g silica gel column, eluting with 0-80% ethyl acetate in petroleum) to give benzyl 3-(3-(5-(3-(5-(2-(3-(3-ethoxy-3-oxopropyl)phenyl)-1-hydroxypropan-2-yl)-1H-imidazol-2-yl)-4-fluorophenoxy)-6-fluoro-1H-indol-4-yl)propoxy)-propanoate (1.5 g, 57%) as oil. MS (ESI): 766 m/z [M+H]⁺, retention time: 1.87 minutes, purity: 87% (214 nm) (LC-MS method 2). ¹H NMR (400 MHz, CD₃OD) δ 7.48 (s, 1H), 7.36-7.24 (m, 6H), 7.19 (d, J=7.7 Hz, 1H), 7.16-7.08 (m, 4H), 7.05 (d, J=6.7 Hz, 1H), 6.93 (s, 1H), 6.82 (s, 1H), 6.54 (d, J=3.0 Hz, 1H), 5.10 (s, 2H), 4.05 (q, J=7.1 Hz, 2H), 3.63 (t, J=6.1 Hz, 2H), 3.40 (t, J=6.3 Hz, 2H), 2.89-2.82 (m, 4H), 2.59-2.52 (m, 4H), 1.93-1.78 (m, 2H), 1.66 (s, 3H), 1.26 (t, J=7.1 Hz, 3H), 1.17 (t, J=7.1 Hz, 3H) ppm.

Benzyl 3-(3-(5-(3-(5-(2-(3-(3-ethoxy-3-oxopropyl)phenyl)-1-oxopropan-2-yl)-1H-imidazol-2-yl)-4-fluorophenoxy)-6-fluoro-1H-indol-4-yl)propoxy)propanoate

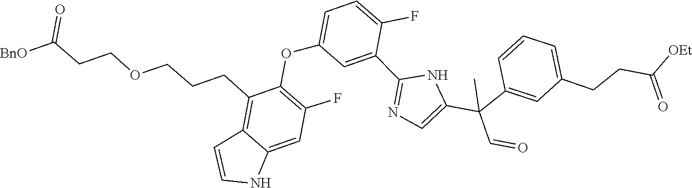 [SMILES: CCOC(=O)CCc1cccc(C(C)(C=O)c2cnc(-c3cc(Oc4c(F)cc5[nH]ccc5c4CCCOCCC(=O)OCc4ccccc4)ccc3F)[nH]2)c1]

Step F: To a solution of Step E product (1.48 g, 1.93 mmol) in dimethyl sulfoxide (50 mL) was added stabilized iodoxybenzoic acid (46%, 1.62 g, 2.67 mmol). The mixture was stirred at room temperature overnight. The mixture was quenched with water (100 mL), extracted with ethyl acetate (3×50 mL). The combined organic extracts were washed with saturated sodium bicarbonate, brine, dried over sodium sulfate, and concentrated. The residue was purified by flash chromatography (40 g silica gel column, eluting with 0-80% ethyl acetate in petroleum ether) to give the title product (1.2 g, 81%) as a solid. MS (ESI): 764 m/z [M+H]⁺, retention time: 1.45 minutes, purity: 72% (214 nm) (LC-MS method 15).

Benzyl 3-(3-(5-(3-(5-(2-(3-(3-ethoxy-3-oxopropyl)phenyl)-3-hydroxy-4-nitrobutan-2-yl)-1H-imidazol-2-yl)-4-fluorophenoxy)-6-fluoro-1H-indol-4-yl)propoxy)propanoate

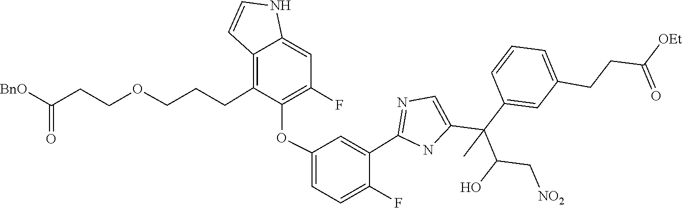

Step G: To a stirred solution of Step F product (1.2 g, 1.1 mmol) in nitromethane (10 mL) was added triethylamine (5 mL). The reaction was stirred at room temperature overnight. The mixture was concentrated to give the crude title compound (1.3 g) as an oil. MS (ESI): 825 m/z [M+H]⁺, retention time: 1.97 minutes, purity: 67% (214 nm) (LC-MS method 2).

3-(3-(5-(3-(5-(4-Amino-2-(3-(3-ethoxy-3-oxopropyl)phenyl)-3-hydroxybutan-2-yl)-1H-imidazol-2-yl)-4-fluorophenoxy)-6-fluoro-1H-indol-4-yl)propoxy)propanoic acid

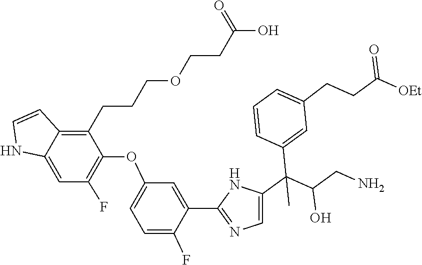 [SMILES: CCOC(=O)CCc1cccc(C(C)(c2cnc(-c3cc(Oc4c(F)cc5[nH]ccc5c4CCCOCCC(=O)O)ccc3F)[nH]2)C(O)CN)c1]

Step H: To a stirred solution of Step G product (1.3 g, 1.58 mmol) in tetrahydrofuran (15 mL) was added Ranney nickel (1.3 g). The mixture was purged with hydrogen and stirred at room temperature for 3 hours. Another portion of Ranney Ni (1.3 g) was added. The mixture was stirred at room temperature for another 4 fours under hydrogen balloon. The mixture was filtered and rinsed with methanol (10 mL×2). The filtrate was concentrated. The residue was purified by flash chromatography (20 g silica gel column, eluting with 0-100% methanol in dichloromethane) to give 0.2 g of title product and 0.6 g of benzyl ester-amine.

The benzyl ester amine was re-submitted to hydrogenation as follows:

To a stirred solution of the benzyl ester above (0.6 g, 0.755 mmol) in ethanol (10 mL) was added 10% Pd—C (0.3 g). The mixture was stirred at room temperature under hydrogen balloon for 3 hours. The mixture was filtered through a pad of Celite and rinsed the filter cake with ethanol (2×10 mL). The filtrate was concentrated to give the crude title compound (0.5 g, yield 94%) as a solid. Total of 0.7 g of title compound obtained. MS (ESI): 705 m/z [M+H]⁺, retention time: 1.06 minutes, purity: 65% (214 nm) (LC-MS method 15).

Compound 75: 3-[3-(24,30-Difluoro-6-methyl-13,26,32-trioxa-3,9,21,33-tetrazahexacyclo[25.3.1.12,5.17,10.017,25.018,22]tritriaconta-1(31),2,4,7,9,17,19,22,24,27,29-undecaen-6-yl)phenyl]propanoic acid

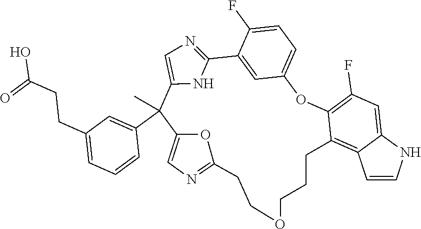 [SMILES: CC1(c2cccc(CCC(=O)O)c2)C2=CN=C(CCOCCCc3c(c(F)cc4[nH]ccc34)Oc3ccc(F)c(c3)-c3ncc1[nH]3)O2]

Exchanging (E)-3-(5-(3-(5-(7-amino-6-hydroxy-2-(3-iodophenyl)heptan-2-yl)-1H-imidazol-2-yl)-4-fluorophenoxy)-6-fluoro-1H-indol-4-yl)acrylic acid (Step C product of Example 21) with 3-(3-(5-(3-(5-(4-amino-2-(3-(3-ethoxy-3-oxopropyl)phenyl)-3-hydroxybutan-2-yl)-1H-imidazol-2-yl)-4-fluorophenoxy)-6-fluoro-1H-indol-4-yl)propoxy)propanoic acid (Step H product of this Example, 0.5 g, 0.709 mol), the reaction procedure sequence (Steps D, G, H and I) described for Example 21 was used to prepare the title compound as a racemic mixture (5.7 mg, 12%). MS (ESI): 639 m/z [M+H]⁺, retention time: 1.69 minutes, purity: 98% (214 nm) (LC-MS method 2). ¹H NMR (400 MHz, CD₃OD) δ 8.39 (s, 1H), 7.45 (s, 1H), 7.25 (d, J=3.2 Hz, 1H), 7.22 (d, J=7.8 Hz, 1H), 7.20-6.98 (m, 5H), 6.93 (s, 1H), 6.82 (s, 1H), 6.49 (d, J=3.2 Hz, 1H), 3.73-3.62 (m, 2H), 3.38 (t, J=7.3 Hz, 2H), 2.88 (t, J=7.7 Hz, 4H), 2.80 (t, J=7.2 Hz, 2H), 2.54 (t, J=7.4 Hz, 2H), 2.02 (d, J=10.7 Hz, 3H), 1.92-1.75 (m, 2H).

Example 76. Compound 76. 3-[3-(24,30-Difluoro-6-methyl-13,26-dioxa-3,9,21,32,33-pentazahexacyclo[25.3.1.12,5.17,10.017,25.018,22]tritriaconta-1(31),2,4,7,10(32),17,19,22,24,27,29-undecaen-6-yl)phenyl]propanoic acid

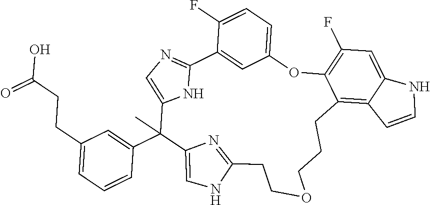 [SMILES: CC1(c2cccc(CCC(=O)O)c2)c2c[nH]c(n2)CCOCCCc2c(c(F)cc3[nH]ccc23)Oc2ccc(F)c(c2)-c2ncc1[nH]2]

Exchanging (E)-3-(5-(3-(5-(7-Amino-6-hydroxy-2-(3-iodophenyl)heptan-2-yl)-1H-imidazol-2-yl)-4-fluorophenoxy)-6-fluoro-1H-indol-4-yl)acrylic acid (Step C product of Example 21) with 3-(3-(5-(3-(5-(4-Amino-2-(3-(3-ethoxy-3-oxopropyl)phenyl)-3-hydroxybutan-2-yl)-1H-imidazol-2-yl)-4-fluorophenoxy)-6-fluoro-1H-indol-4-yl)propoxy)propanoic acid (Step H product of Example 75, 0.5 g, 0.709 mol), the reaction procedure sequence (Steps D, G) described for Example 21, followed by the reaction procedure sequence (Steps A and B) described for Example 22 was used to prepare the title compound as a racemic mixture (22.8 mg). MS (ESI): 638 m/z [M+H]⁺, retention time: 1.74 minutes, purity: 96% (214 nm) (LC-MS method 4). ¹H NMR (400 MHz, CD₃OD) δ 7.33-7.31 (m, 1H), 7.15 (d, J=3.2 Hz, 1H), 7.11 (d, J=10.7 Hz, 1H), 7.06-6.90 (m, 6H), 6.78 (s, 1H), 6.73 (d, J=7.7 Hz, 1H), 6.40 (d, J=3.2 Hz, 1H), 3.53-3.48 (m, 2H), 3.44-3.37 (m, 1H), 3.34-3.29 (m, 1H), 2.95-2.84 (m, 1H), 2.79-2.74 (m, 3H), 2.67 (t, J=7.6 Hz, 2H), 2.28 (t, J=7.6 Hz, 2H), 1.84 (s, 3H), 1.82-1.60 (m, 2H).

Example 77. Compound 77. 3-[3-(24,30-Difluoro-6,11,11-trimethyl-14,26-dioxa-3,21,32,33-tetrazahexacyclo[25.3.1.12,5.112,15.017,25.018,22]tritriaconta-1(31),2,4,12,15(32),17,19,22,24,27,29-undecaen-6-yl)phenyl]propanoic acid

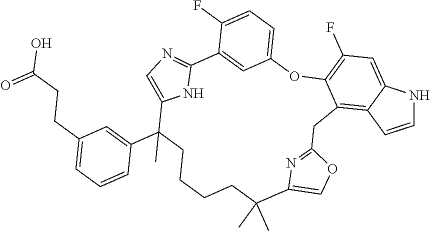 [SMILES: CC1(C)CCCCC(C)(c2cccc(CCC(=O)O)c2)c2cnc([nH]2)-c2cc(ccc2F)Oc2c(F)cc3[nH]ccc3c2Cc2nc1co2]

Exchanging 9-bromo-7-(3-iodophenyl)-7-methyl-8-oxononanenitrile (Intermediate 2-32) with 4-(9-bromo-7-(3-bromophenyl)-2,7-dimethyl-8-oxononan-2-yl)oxazolidin-2-one (Intermediate 2-44, 0.839 g, 2.25 mmol) and 2-fluoro-5-((6-fluoro-4-((2-oxooxazolidin-5-yl)methyl)-1H-indol-5-yl)oxy)benzimidamide (Intermediate 6-8) with ethyl 2-(5-(3-carbamimidoyl-4-fluorophenoxy)-6-fluoro-1H-indol-4-yl)acetate (Intermediate 29, 1.1 g, 2.25 mmol), the reaction procedure sequence (Steps A to C) described for Example 52 was used to prepare the title compound (2.2 mg) as a racemic mixture. MS (ESI): 665 m/z [M+H]⁺, retention time: 1.55 minutes, purity: >99% (214 nm) (LC-MS method 9). ¹H NMR (400 MHz, CD₃OD). ¹H NMR (400 MHz, CD₃OD) δ 7.36 (s, 1H), 7.32-7.25 (m, 3H), 7.19-7.02 (m, 6H), 6.89-6.87 (m, 1H), 6.48 (d, J=2.8 Hz, 1H), 4.27 (s, 2H), 2.88-2.84 (m, 2H), 2.51-2.48 (m, 2H), 2.23-2.12 (m, 2H), 1.76-1.56 (m, 5H), 1.44-1.06 (m, 10H) ppm.

Example 78. Compound 78A and Compound 78B. Enantiomers 1 and 2 of 3-[3-(24,30-difluoro-6,11,11-trimethyl-26-oxa-3-thia-13,14,15,21,33-pentazahexacyclo-[25.3.1.12,5.112,15.017,25.018,22]tritriaconta-1(31),2(33),4,12(32),13,17,19,22,24,27,29-undecaen-6-yl)phenyl]propanoic acid

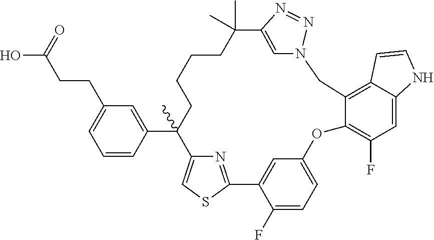 [SMILES: CC1(C)CCCCC(C)(c2cccc(CCC(=O)O)c2)c2csc(n2)-c2cc(ccc2F)Oc2c(F)cc3[nH]ccc3c2Cn2cc1nn2]

Methyl 3-(3-(2-(2-(2-fluoro-5-((6-fluoro-4-(hydroxymethyl)-1H-indol-5-yl)oxy)phenyl)thiazol-4-yl)-7,7-dimethylnon-8-yn-2-yl)phenyl)propanoate

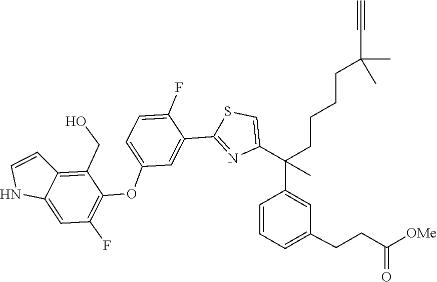 [SMILES: C#CC(C)(C)CCCCC(C)(c1cccc(CCC(=O)OC)c1)c1csc(-c2cc(Oc3c(F)cc4[nH]ccc4c3CO)ccc2F)n1]

Step A: To a stirred solution of 2-fluoro-5-((6-fluoro-4-(hydroxymethyl)-1H-indol-5-yl)oxy)-benzothioamide (Intermediate 56-1, 0.65 g, 1.9 mmol) in 4 mL of dimethylformamide was added a solution of methyl 3-(3-(1-bromo-3,8,8-trimethyl-2-oxodec-9-yn-3-yl)phenyl)propanoate (Intermediate 2-45, 0.84 g, 1.9 mmol) in 4 mL of dimethylformamide. The reaction was stirred at 80° C. overnight and cooled to room temperature. The solution was partitioned between water (20 mL) and ethyl acetate (20 ml). The separated organic layer, combined with two additional ethyl acetate extracts (2×30 mL), was washed with brine, dried over sodium sulfate and concentrated. The residue was purified by column chromatography to give the title compound (0.46 g, 35%) as a yellow oil. MS (ESI): 671 m/z [M+H]⁺, retention time: 2.43 minutes, purity: 93% (214 nm) (LC-MS method 9).

Methyl 3-(3-(2-(2-(5-((4-(azidomethyl)-6-fluoro-1H-indol-5-yl)oxy)-2-fluorophenyl)thiazol-4-yl)-7,7-dimethylnon-8-yn-2-yl)phenyl)propanoate

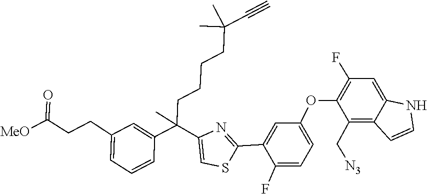 [SMILES: C#CC(C)(C)CCCCC(C)(c1cccc(CCC(=O)OC)c1)c1csc(-c2cc(Oc3c(F)cc4[nH]ccc4c3CN=[N+]=[N-])ccc2F)n1]

Step B: To a stirred solution of Step A product (0.46 g, 0.7 mmol) in 10 mL of tetrahydrofuran was added diphenyl-phosphoryl azide (0.75 g, 2.7 mmol) and 1,8-diazabicyclo[5.4.0]undec-7-ene (0.21 g, 1.4 mmol). The mixture was stirred at reflux overnight and cooled to room temperature. The solution was partitioned between water (20 mL) and ethyl acetate (30 ml). The separated organic layer, combined with two additional ethyl acetate extracts (2×30 mL), was washed with brine, dried over sodium sulfate and concentrated. The residue was purified by column chromatography to give the title compound (0.26 g, 55%) as a brown sticky oil. MS (ESI): 718 m/z [M+Na]⁺, retention time: 2.79 minutes, purity: >99% (254 nm) (LC-MS method 2).

Compounds 78A and 78B: Enantiomers 1 and 2 of 3-[3-(24,30-difluoro-6,11,11-trimethyl-26-oxa-3-thia-13,14,15,21,33-pentazahexacyclo-[25.3.1.12,5.112,15.017,25.018,22]tritriaconta-1(31),2(33),4,12(32),13,17,19,22,24,27,29-undecaen-6-yl)phenyl]propanoic acid

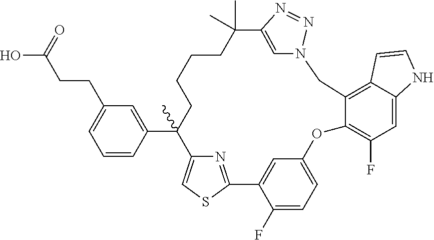 [SMILES: CC1(C)CCCCC(C)(c2cccc(CCC(=O)O)c2)c2csc(n2)-c2cc(ccc2F)Oc2c(F)cc3[nH]ccc3c2Cn2cc1nn2]

Exchanging 4-(azidomethyl)-6-fluoro-5-(4-fluoro-3-(5-(2-(3-iodophenyl)oct-7-yn-2-yl)-1H-imidazol-2-yl)phenoxy)-1H-indole (Step A product of Example 2) with methyl 3-(3-(2-(2-(5-((4-(azidomethyl)-6-fluoro-1H-indol-5-yl)oxy)-2-fluorophenyl)thiazol-4-yl)-7,7-dimethylnon-8-yn-2-yl)phenyl)propanoate (Step B product of this example, 238 mg, 0.34 mmol), the reaction procedure sequence (steps B and E) described for Example 2 was used to prepare the title compounds. The methyl ester macrocycle (183 mg), obtained from corresponding B of Example 2, was subject to chiral Prep HPLC separation. The first eluent (94 mg, 43%), enantiomer 1, was further hydrolyzed to Compound 78A (75 mg, 81%); The second eluent (92 mg, 42%), was further hydrolyzed to Compound 78B (83 mg, 92%), based on the conditions described in Step E of Example 2. (Chiral SFC analytical conditions: Injection Volume: 5 μL; Column: OJ-H 4.6*100 mm, 5 μm; Column temperature: 39.9° C.; Co-Solvent: methanol (0.2% methanol ammonia); Carbon dioxide Flow Rate: 3.2; Co-Solvent Flow rate: 0.8; Co-Solvent %: 20; Total Flow: 4; Front Pressure: 148; Back pressure: 122; Pressure Drop: 26; PDA Start Wavelength: 214 nm; PDA Stop Wavelength: 359 nm.)

Compound 78A: MS (ESI): 704 m/z [M+Na]⁺, retention time: 3.52 minutes, purity: >99% (254 nm) (LC-MS method 17). ¹H NMR (400 MHz, CD₃OD) δ 7.44-7.29 (m, 7H), 7.13-7.09 (m, 2H), 7.01-6.99 (m, 2H), 6.69 (d, J=3.2 Hz, 1H), 6.04-5.97 (m, 1H), 5.81 (J=14.4 Hz, 1H), 2.86 (t, J=8.4 Hz, 2H), 2.51 (t, J=8.8 Hz, 2H), 2.10 (t, J=13.2 Hz, 1H), 1.77-1.71 (m, 1H), 1.61 (s, 3H), 1.42-1.33 (m, 2H), 1.19-1.16 (m, 4H), 1.06 (s, 3H), 0.95-0.85 (m, 1H), 0.72-0.62 (m, 1H), 0.49-0.39 (m, 1H) ppm.

Compound 78B: MS (ESI): 704 m/z [M+Na]⁺, retention time: 3.52 minutes, purity: >99% (254 nm) (LC-MS method 17). ¹H NMR (400 MHz, CD₃OD) δ 7.42-7.29 (m, 7H), 7.14-7.09 (m, 2H), 7.01-6.98 (m, 2H), 6.69 (d, J=3.2 Hz, 1H), 6.04-5.97 (m, 1H), 5.81 (J=14.4 Hz, 1H), 2.85 (t, J=8.4 Hz, 2H), 2.54 (t, J=8.4 Hz, 2H), 2.10 (t, J=13.2 Hz, 1H), 1.78-1.72 (m, 1H), 1.61 (s, 3H), 1.40-1.28 (m, 2H), 1.19-1.16 (m, 4H), 1.06 (s, 3H), 0.92-0.85 (m, 1H), 0.73-0.67 (m, 1H), 0.51-0.39 (m, 1H) ppm.

Example 79. Compound 79. ethyl 3-[3-(10,16-difluoro-22-methyl-12-oxa-1,7,19,35,36-pentazaheptacyclo[26.6.1.113,17.118,21.03,11.04,8.029,34]heptatriaconta-3,5,8,10,13,15,17(37),18,20,28(35),29(34),30,32-tridecaen-22-yl)phenyl]propanoate

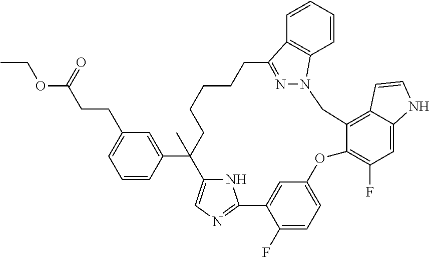 [SMILES: CCOC(=O)CCc1cccc(C2(C)CCCCCc3nn(c4ccccc34)Cc3c(c(F)cc4[nH]ccc34)Oc3ccc(F)c(c3)-c3ncc2[nH]3)c1]

Exchanging 5-((4-((4-bromothiazol-2-yl)methyl)-6-fluoro-1H-indol-5-yl)oxy)-2-fluorobenzimidamide (Intermediate 30) with 5-((4-((3-bromo-1H-indazol-1-yl)methyl)-6-fluoro-1H-indol-5-yl)oxy)-2-fluorobenzimidamide (Intermediate 68, 282 mg, 0.48 mmol) and methyl (E)-3-(3-(1-bromo-3-methyl-2-oxooct-7-yn-3-yl)phenyl)acrylate (Intermediate 2-20) with methyl (E)-3-(3-(1-chloro-3-methyl-2-oxooct-7-yn-3-yl)phenyl)acrylate (Intermediate 2-46, 201 mg, 0.53 mmol), the reaction procedure sequence (Steps A to D) described for Example 36 was used to prepare the title compound (35 mg, a white solid). MS (ESI): 714 m/z [M+H]⁺, retention time: 1.46 minutes, purity: 97% (254 nm) (LC-MS method 2). ¹H NMR (400 MHz, CDCl₃) δ 7.51-6.88 (m, 12H), 6.83-6.71 (m, 1H), 6.61 (s, 2H), 5.86-5.62 (m, 2H), 4.03 (q, J=7.2 Hz, 2H), 2.87-2.79 (m, 2H), 2.56-2.44 (m, 4H), 2.29-2.23 (m, 1H), 2.05-1.99 (m, 1H), 1.70-1.64 (m, 2H), 1.55-1.50 (m, 4H), 1.18-1.11 (m, 4H), 0.99-0.94 (m, 2H).

Example 80. Compound 80. 3-[3-(24,30-Difluoro-6,11-dimethyl-10,26,32-trioxa-3,13,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

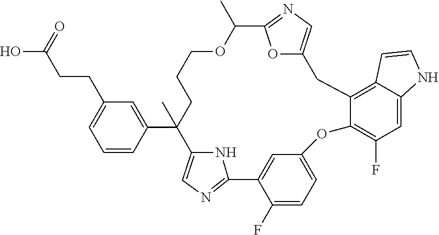 [SMILES: CC1OCCCC(C)(c2cccc(CCC(=O)O)c2)c2cnc([nH]2)-c2cc(ccc2F)Oc2c(F)cc3[nH]ccc3c2CC2=CN=C1O2]

Exchanging 9-bromo-7-(3-iodophenyl)-7-methyl-8-oxononanenitrile (Intermediate 2-32) with methyl 2-((6-bromo-4-(3-bromophenyl)-4-methyl-5-oxohexyl)oxy)propanoate (Intermediate, 33-1, 1.84 g, 3.88 mmol), the reaction procedure sequence (Steps A to E) described for Example 52 was used to prepare the title compound (3 mg) as a racemic mixture, without performing Heck coupling, hydrogenation and hydrolysis in Step E. MS (ESI): 653 m/z [M+H]⁺, retention time: 1.66 minutes, purity: 95% (214 nm) (LC-MS method 18), (mixture of 4 diastereomers, originating from two chiral centers).

Example 81. Compound 81. 3-[3-(24,30-Difluoro-6,11,11-trimethyl-10,26,32-trioxa-3,13,21,33-tetrazahexacyclo[25.3.1.12,5.112,15.017,25.018,22]tritriaconta-1(31),2,4,12,14,17,19,22,24,27,29-undecaen-6-yl)phenyl]propanoic acid

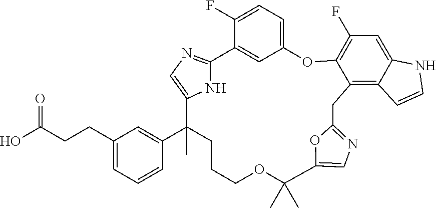 [SMILES: CC1(C)OCCCC(C)(c2cccc(CCC(=O)O)c2)c2cnc([nH]2)-c2cc(ccc2F)Oc2c(F)cc3[nH]ccc3c2CC2=NC=C1O2]

Exchanging 9-bromo-7-(3-iodophenyl)-7-methyl-8-oxononanenitrile (Intermediate 2-32) with methyl 2-((6-bromo-4-(3-bromophenyl)-4-methyl-5-oxohexyl)oxy)-2-methylpropanoate (Intermediate 33-2, 0.6 g, 0.00129 mol), the reaction procedure sequence (Steps A to C) described for Example 52 was used to prepare the title compound (2.5 mg) as a racemic mixture. MS (ESI): 667 m/z [M+H]⁺, retention time: 1.13 minutes, purity: >99% (214 nm) (LC-MS method 9). ¹H NMR (400 MHz, CD₃OD) δ 7.23-7.19 (m, 1H), 7.14-7.08 (m, 3H), 7.00-6.97 (m, 2H), 6.89-6.85 (m, 3H), 6.79-6.75 (m, 1H), 6.59-6.55 (m, 1H), 6.02 (s, 1H), 4.17-4.11 (m, 2H), 3.22-3.20 (m, 2H), 2.75-2.73 (m, 1H), 2.72 (t, J=7.6 Hz, 2H), 2.39 (t, J=7.6 Hz, 2H), 3.36-3.34 (m, 1H), 1.84-1.80 (m, 2H), 1.37 (s, 3H), 1.34 (s, 3H), 1.25 (s, 3H).

Example 82. Compound 82. 3-[3-(24,30-difluoro-6,11,11,14-tetramethyl-26-oxa-3,14,21,32,33-pentazahexacyclo[25.3.1.12,5.112,15.017,25.018,22]tritriaconta-1(31),2,4,12,15(32),17,19,22,24,27,29-undecaen-6-yl)phenyl]propanoic acid

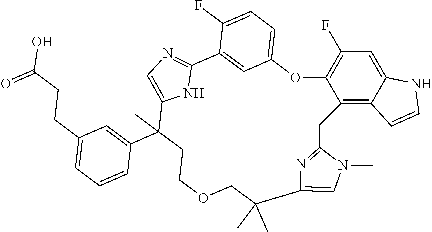 [SMILES: Cn1cc2nc1Cc1c(c(F)cc3[nH]ccc13)Oc1ccc(F)c(c1)-c1ncc([nH]1)C(C)(c1cccc(CCC(=O)O)c1)CCOCC2(C)C]

Exchanging 2-fluoro-5-((6-fluoro-4-((3-methyl-2-oxooxazolidin-5-yl)methyl)-1H-indol-5-yl)oxy)benzimidamide (Intermediate 6-10) with methyl 2-(5-(3-carbamimidoyl-4-fluorophenoxy)-6-fluoro-1H-indol-4-yl)acetate (intermediate 29, 1.49 g, 3.8 mmol) and 9-chloro-7-(3-iodophenyl)-7-methyl-8-oxononanenitrile (Intermediate 2-32) with 5-(9-bromo-7-(3-bromophenyl)-2,7-dimethyl-8-oxononan-2-yl)-3-methyloxazolidin-2-one (Intermediate 2-47, 2.00 g, 3.78 mmol), the reaction procedure sequence (Steps A to C) described for Example 54 was used to prepare the title compound (65 mg) as a white solid. MS (ESI): 678 m/z [M+H]⁺, retention time: 1.50 minutes, purity: 95% (214 nm) (LC-MS method 5). ¹H NMR (400 MHz, CD₃OD) δ 7.32 (dd, J=5.8, 3.2 Hz, 1H), 7.28-7.20 (m, 2H), 7.08 (t, J=10.0 Hz, 1H), 7.03-6.96 (m, 2H), 6.95-6.84 (m, 5H), 6.21 (d, J=3.0 Hz, 1H), 4.43 (d, J=17.5 Hz, 1H), 4.32 (d, J=17.5 Hz, 1H), 3.47 (s, 3H), 2.70 (t, J=10.8 Hz, 2H), 2.27 (t, J=10.8 Hz, 2H), 2.15-1.91 (m, 2H), 1.75-1.65 (m, 1H), 1.60-1.48 (m, 1H), 1.47-1.21 (m, 4H), 1.06-0.94 (m, 8H), 0.50-0.40 (m, 1H).

Example 83. Compound 83A and Compound 83B. 3-[3-(24,30-Difluoro-11,11-dimethyl-26,32-dioxa-5,13,21,33-tetrazahexacyclo[25.3.1.12,5.112,15.017,25.018,22]tritriaconta-1(31),2(33),3,12,14,17,19,22,24,27,29-undecaen-6-yl)phenyl]propanoic acid

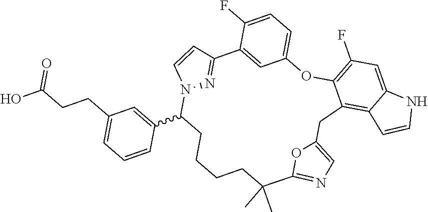

Methyl 7-(3-(5-((4-allyl-6-fluoro-1-tosyl-1H-indol-5-yl)oxy)-2-fluorophenyl)-1H-pyrazol-1-yl)-7-(3-bromophenyl)-2,2-dimethylheptanoate

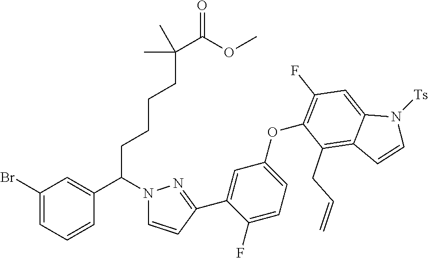 [SMILES: C=CCc1c(Oc2ccc(F)c(-c3ccn(C(CCCCC(C)(C)C(=O)OC)c4cccc(Br)c4)n3)c2)c(F)cc2c1ccn2S(=O)(=O)c1ccc(C)cc1]

Step A: To a stirred solution of methyl 7-bromo-7-(3-bromophenyl)-2,2-dimethylheptanoate (Intermediate 59-1, 1.83 g, 0.0045 mol) in dimethylformamide (30 mL) was added 4-allyl-6-fluoro-5-(4-fluoro-3-(1H-pyrazol-3-yl)phenoxy)-1-tosyl-1H-indole (Intermediate 73, 1.52 g, 0.003 mol) and cesium carbonate (2.95 g, 0.003 mol). The reaction was stirred for one hour. The mixture was partitioned between water (50 mL) and ethyl acetate (150 mL). The separated organic phase was washed with water, brine, dried over sodium sulfate, and concentrated. The residue was purified by silica gel column chromatography (petroleum ether/ethyl acetate=4:1) to give the title compound (2.0 g, 80.0%) as a yellow oil. MS (ESI): 830, 832 m/z [M+H]⁺, retention time: 2.88 minutes, purity: 81% (214 nm) (LC-MS method 6).

Methyl 7-(3-(5-((4-(3-(((benzyloxy)carbonyl)amino)-2-hydroxypropyl)-6-fluoro-1-tosyl-1H-indol-5-yl)oxy)-2-fluorophenyl)-1H-pyrazol-1-yl)-7-(3-bromophenyl)-2,2-dimethylheptanoate

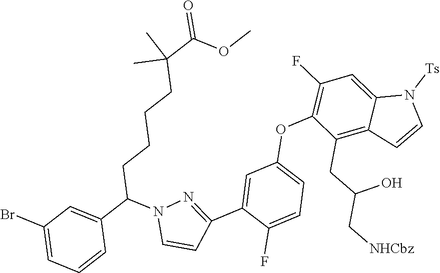 [SMILES: COC(=O)C(C)(C)CCCCC(c1cccc(Br)c1)n1ccc(-c2cc(Oc3c(F)cc4c(ccn4S(=O)(=O)c4ccc(C)cc4)c3CC(O)CNC(=O)OCc3ccccc3)ccc2F)n1]

Step B: To a stirred solution of benzyl ((4-chlorobenzoyl)oxy)carbamate (1.66 g, 0.00542 mol) in acetonitrile (60 mL) was added osmium tetroxide (0.184 g, 0.000722 mol). The mixture was stirred for 0.5 hours, treated with Step A product (3.0 g, 0.00361 mol) and water (6 mL). The reaction was stirred for 20 hours. The solution was partitioned between water (50 mL) and ethyl acetate (100 mL). The separated organic layer was washed with water and brine, dried over sodium sulfate, and concentrated. The residue was purified by silica gel column chromatography (petroleum ether/ethyl acetate=1:1) to give the title compound (2.6 g, 72%) as a yellow solid. MS (ESI): 997, 999 m/z [M+H]⁺, retention time: 2.53 minutes, purity: 50% (214 nm) (LC-MS method 6).

7-(3-(5-((4-(3-Amino-2-hydroxypropyl)-6-fluoro-1H-indol-5-yl)oxy)-2-fluorophenyl)-1H-pyrazol-1-yl)-7-(3-bromophenyl)-2,2-dimethylheptanoic acid

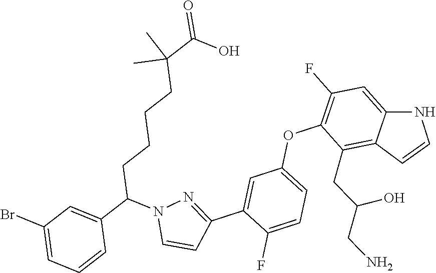 [SMILES: CC(C)(CCCCC(c1cccc(Br)c1)n1ccc(-c2cc(Oc3c(F)cc4[nH]ccc4c3CC(O)CN)ccc2F)n1)C(=O)O]

Step C: To a solution of Step B product (2.6 g, 0.00261 mol) in methanol (40 mL) and water (10 mL) was added lithium hydroxide monohydrate (1.09 g, 0.0261 mol). The reaction was stirred for 16 hours at room temperature, then at 70° C. for 48 hours and concentrated. The residue was acidified with 1 M hydrochloric acid to pH 4-5. The formed precipitate was collected by filtration to afford the title compound (1.5 g, 83% yield) as a yellow solid. MS (ESI): 695, 697 m/z [M+H]⁺, retention time: 1.94 minutes, purity: 30% (214 nm) (LC-MS method 6).

Compounds 83A and 83B: 3-[3-(24,30-Difluoro-11,11-dimethyl-26,32-dioxa-5,13,21,33-tetrazahexacyclo[25.3.1.12,5.112,15.017,25.018,22]tritriaconta-1(31),2(33),3,12,14,17,19,22,24,27,29-undecaen-6-yl)phenyl]propanoic acid

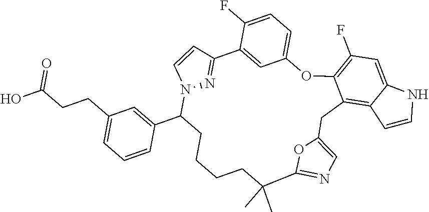

Exchanging (E)-3-(5-(3-(5-(7-Amino-6-hydroxy-2-(3-iodophenyl)heptan-2-yl)-1H-imidazol-2-yl)-4-fluorophenoxy)-6-fluoro-1H-indol-4-yl)acrylic acid (Step C product of Example 21), with 7-(3-(5-((4-(3-Amino-2-hydroxypropyl)-6-fluoro-1H-indol-5-yl)oxy)-2-fluorophenyl)-1H-pyrazol-1-yl)-7-(3-bromophenyl)-2,2-dimethylheptanoic acid (Step C product of this Example, 1.3 g, 0.00187 mol), the reaction procedure sequence (steps D to I) described for Example 21 was used to prepare the title compounds. The racemic acid (100 mg), the corresponding Step I product, was subject to chiral prep-HPLC separation under the following condition: Instrument: Gilson-281; Column: OZ 20*250 mm, 10 μm; Mobile Phase: n-Hexane (0.1% diethylamine): ethanol (0.1% diethylamine)=70:30; Run time per injection: 16 minutes; Injection: 0.8 mL; Sample solution: 100 mg in 11 mL. methanol. The first eluent, enantiomer 1, is designated as Compound 83A (26 mg, 26%, 100% ee); and the second eluent, Enantiomer 2, is designated as Compound 83B (28 mg, 28%, 100% ee).

Compound 83A: MS (ESI): 651 m/z [M+H]⁺, retention time: 2.29 minutes, purity: >99% (214 nm) (LC-MS method 6). ¹H NMR (400 MHz, CD₃OD) δ 7.57 (d, J=2.4 Hz, 1H), 7.34 (d, J=3.2 Hz, 1H), 7.31 (d, J=7.2 Hz, 1H), 7.21-7.10 (m, 5H), 7.07-7.03 (m, 2H), 6.71 (d, J=1.2 Hz, 1H), 6.62 (dd, J=4.4, 2.4 Hz, 1H), 6.12 (s, 1H), 5.16 (dd, J=12.0, 3.2 Hz, 1H), 4.29-4.10 (m, 2H), 2.89 (t, J=7.6 Hz, 2H), 2.56 (t, J=7.6 Hz, 2H), 2.33-2.29 (m, 1H), 1.87-1.83 (m, 1H), 1.49-1.45 (m, 1H), 1.39-1.26 (m, 2H), 1.22 (s, 3H), 1.14 (s, 3H), 1.02-0.98 (m, 1H), 0.92-0.85 (m, 1H), 0.62-0.59 (m, 1H).

Compound 83B: MS (ESI): 651 m/z [M+H]⁺, retention time: 2.29 minutes, purity: >99% (214 nm) (LC-MS method 6). ¹H NMR (400 MHz, CD₃OD) δ 7.57 (d, J=2.4 Hz, 1H), 7.34 (d, J=3.2 Hz, 1H), 7.31 (d, J=7.2 Hz, 1H), 7.21-7.10 (m, 5H), 7.07-7.03 (m, 2H), 6.71 (d, J=1.2 Hz, 1H), 6.62 (dd, J=4.4, 2.4 Hz, 1H), 6.12 (s, 1H), 5.16 (dd, J=12.0, 3.2 Hz, 1H), 4.29-4.10 (m, 2H), 2.89 (t, J=7.6 Hz, 2H), 2.56 (t, J=7.6 Hz, 2H), 2.33-2.29 (m, 1H), 1.87-1.83 (m, 1H), 1.49-1.45 (m, 1H), 1.39-1.26 (m, 2H), 1.22 (s, 3H), 1.14 (s, 3H), 1.02-0.98 (m, 1H), 0.92-0.85 (m, 1H), 0.62-0.59 (m, 1H).

Example 84. Compound 84. 3-[3-(24,30-Difluoro-10,10-dimethyl-26-oxa-5,12,13,21,33-pentazahexacyclo[25.3.1.12,5.112,15.017,25.018,22]tritriaconta-1(31),2(33),3,13,15(32),17,19,22,24,27,29-undecaen-6-yl)phenyl]propanoic acid

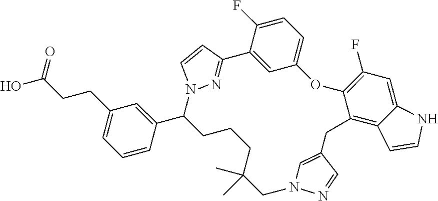 [SMILES: CC1(C)CCCC(c2cccc(CCC(=O)O)c2)n2ccc(n2)-c2cc(ccc2F)Oc2c(F)cc3[nH]ccc3c2Cc2cnn(c2)C1]

and

Example 85. Compound 84-3. 3-[3-[(8Z)-24,30-Difluoro-10,10-dimethyl-26-oxa-5,12,13,21,33-pentazahexacyclo[25.3.1.12,5.112,15.017,25.018,22]tritriaconta-1(31),2(33),3,8,13,15(32),17,19,22,24,27,29-dodecaen-6-yl]phenyl]propanoic acid

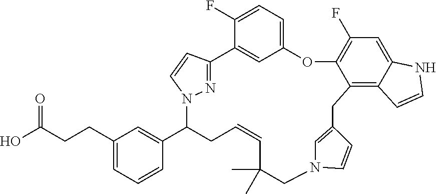 [SMILES: CC1(C)/C=C\CC(c2cccc(CCC(=O)O)c2)n2ccc(n2)-c2cc(ccc2F)Oc2c(F)cc3[nH]ccc3c2Cc2ccn(c2)C1]

and

Compound 84-H4. 3-[3-(25,31-Difluoro-11,11-dimethyl-27-oxa-5,6,13,14,22-pentazahexacyclo[26.3.1.113,16.02,6.018,26.019,23]tritriaconta-1(32),2,4,14,16(33),18,20,23,25,28,30-undecaen-7-yl)phenyl]propanoic acid

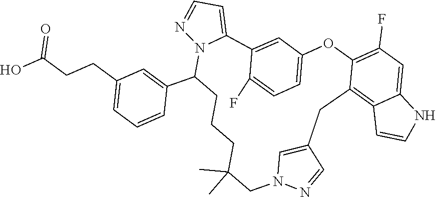 [SMILES: CC1(C)CCCC(c2cccc(CCC(=O)O)c2)n2nccc2-c2cc(ccc2F)Oc2c(F)cc3[nH]ccc3c2Cc2cnn(c2)C1]

Mixture of ethyl 3-(3-(1-(3-(5-((4-(((tert-butyldiphenylsilyl)oxy)methyl)-6-fluoro-1-tosyl-1H-indol-5-yl)oxy)-2-fluorophenyl)-1H-pyrazol-1-yl)but-3-en-1-yl)phenyl)propanoate and ethyl 3-(3-(1-(5-(5-((4-(((tert-butyldiphenylsilyl)oxy)methyl)-6-fluoro-1-tosyl-1H-indol-5-yl)oxy)-2-fluorophenyl)-1H-pyrazol-1-yl)but-3-en-1-yl)phenyl)propanoate

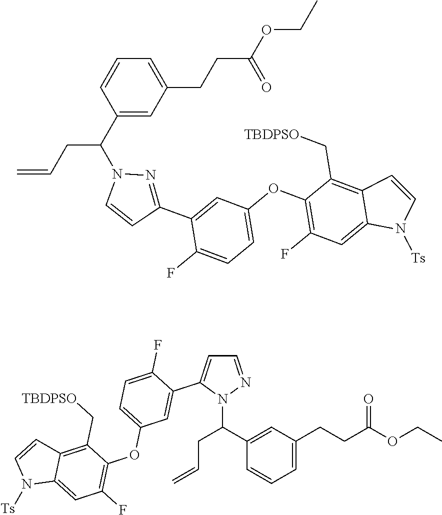

Step A: To a stirred and degassed solution of 4-(((tert-butyldiphenylsilyl)oxy)methyl)-6-fluoro-5-(4-fluoro-3-(1H-pyrazol-3-yl)phenoxy)-1-tosyl-1H-indole (Intermediate 76, 1.85 g, 2.52 mmol) in 1,4-dioxane (15 mL) was added ethyl 3-(3-(1-hydroxybut-3-en-1-yl)phenyl)propanoate (Intermediate 75, 751 mg, 3.02 mmol) and (cyanomethylene)tributylphosphorane (1.58 g, 6.55 mmol) in a microwave reaction vial. The mixture was heated to 150° C. for 30 minutes in a microwave reactor. The residue was partitioned between water (20 mL) and ethyl acetate (30 mL). The separated organic phase, combined with two additional ethyl acetate extracts (2×30 mL), was washed with brine, dried over sodium sulfate and concentrated. The residue was purified by silica gel column chromatography (eluted with petroleum ether/ethyl acetate=10/1) to give the title compound (1.9 g, 75%, inseparable mixture) as a yellow solid. MS (ESI): 964 m/z [M+H]⁺, retention time: 2.03 minutes, purity: 81% (214 nm) (LC-MS method 9).

Mixture of ethyl 3-(3-(1-(3-(2-fluoro-5-((6-fluoro-4-(hydroxymethyl)-1-tosyl-1H-indol-5-yl)oxy)phenyl)-1H-pyrazol-1-yl)but-3-en-1-yl)phenyl)propanoate and ethyl 3-(3-(1-(5-(2-fluoro-S-((6-fluoro-4-(hydroxymethyl)-1-tosyl-1H-indol-5-yl)oxy)phenyl)-1H-pyrazol-1-yl)but-3-en-1-yl)phenyl)propanoate

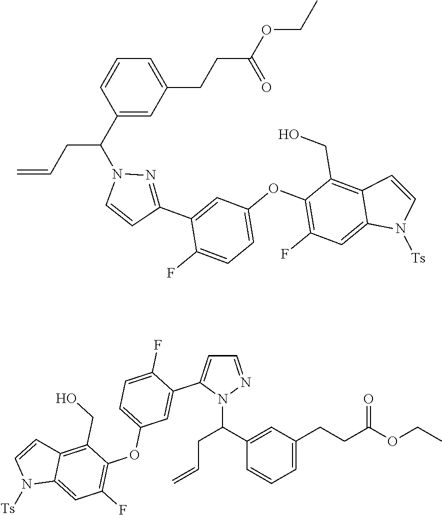 [SMILES: C=CCC(c1cccc(CCC(=O)OCC)c1)n1ccc(-c2cc(Oc3c(F)cc4c(ccn4S(=O)(=O)c4ccc(C)cc4)c3CO)ccc2F)n1 ; C=CCC(c1cccc(CCC(=O)OCC)c1)n1nccc1-c1cc(Oc2c(F)cc3c(ccn3S(=O)(=O)c3ccc(C)cc3)c2CO)ccc1F]

Step B: To a stirred solutions of Step A product (4.3 g, 4.5 mmol) in tetrahydrofuran (40 mL) was added tetrabutylammonium fluoride (1 M in tetrahydrofuran, 13.4 mL, 13.4 mmol). The resulting mixtures were stirred at room temperature for 16 hours and concentrated. The residue was acidified with 1 N hydrochloric acid to pH ˜6 and extracted with ethyl acetate (3×80 mL). The combined organic phases were dried over anhydrous sodium sulfate, filtered and concentrated. The residue was purified by silica gel column chromatography (eluting with petroleum ether/ethyl acetate=4/1) to give the title compound (2.76 g, 85%, inseparable mixture) as a yellow oil. MS (ESI): 726 m/z [M+H]⁺, retention time: 1.70 minutes, purity: 86% (214 nm) (LC-MS method 19).

Mixture of Ethyl 3-(3-(1-(3-(5-((4-(bromomethyl)-6-fluoro-1-tosyl-1H-indol-5-yl)oxy)-2-fluorophenyl)-1H-pyrazol-1-yl)but-3-en-1-yl)phenyl)propanoate and ethyl 3-(3-(1-(5-(5-((4-(bromomethyl)-6-fluoro-1-tosyl-1H-indol-5-yl)oxy)-2-fluorophenyl)-1H-pyrazol-1-yl)but-3-en-1-yl)phenyl)-propanoate

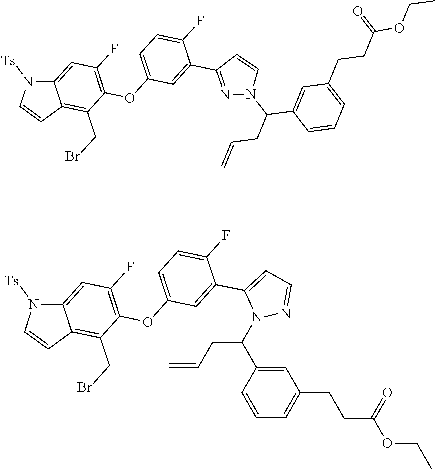 [SMILES: C=CCC(c1cccc(CCC(=O)OCC)c1)n1ccc(-c2cc(Oc3c(F)cc4c(ccn4S(=O)(=O)c4ccc(C)cc4)c3CBr)ccc2F)n1 ; C=CCC(c1cccc(CCC(=O)OCC)c1)n1nccc1-c1cc(Oc2c(F)cc3c(ccn3S(=O)(=O)c3ccc(C)cc3)c2CBr)ccc1F]

Step C: To a stirred and cooled (0° C.) solution of Step B product (2.65 g, 3.65 mmol) in dichloromethane (30 mL) was added 2,6-lutidine (1.17 g, 11 mmol), phosphorus tribromide (1.98 g, 7.3 mmol). The mixture was stirred at room temperature overnight, quenched with brine (30 mL), and acidified with 1 N hydrochloric acid to pH ˜6. The mixture was extracted with ethyl acetate (3×80 mL). The combined organic phase was dried over sodium sulfate and concentrated. The residue was purified by silica gel column chromatography (eluting with petroleum ether/ethyl acetate=4/1) to give the title compound (2.3 g, 80%, inseparable mixture) as a yellow oil. MS (ESI): 788,790 m/z [M+H]⁺, retention time: 1.70 minutes, purity: 86% (214 nm) (LC-MS method 19).

Product 1: ethyl 3-(3-(1-(3-(5-((4-((1-(2,2-dimethylbut-3-en-1-yl)-1H-pyrazol-4-yl)methyl)-6-fluoro-1-tosyl-1H-indol-5-yl)oxy)-2-fluorophenyl)-1H-pyrazol-1-yl)but-3-en-1-yl)phenyl)propanoate

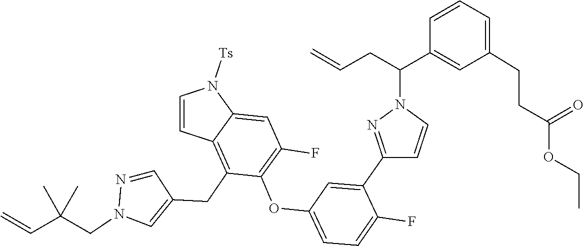 [SMILES: C=CCC(c1cccc(CCC(=O)OCC)c1)n1ccc(-c2cc(Oc3c(F)cc4c(ccn4S(=O)(=O)c4ccc(C)cc4)c3Cc3cnn(CC(C)(C)C=C)c3)ccc2F)n1]

Product 2: ethyl 3-(3-(1-(5-(5-((4-((1-(2,2-dimethylbut-3-en-1-yl)-1H-pyrazol-4-yl)methyl)-6-fluoro-1-tosyl-1H-indol-5-yl)oxy)-2-fluorophenyl)-1H-pyrazol-1-yl)but-3-en-1-yl)phenyl)propanoate

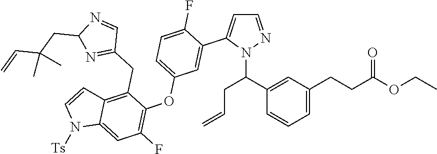 [SMILES: C=CCC(c1cccc(CCC(=O)OCC)c1)n1nccc1-c1cc(Oc2c(F)cc3c(ccn3S(=O)(=O)c3ccc(C)cc3)c2CC2=NC(CC(C)(C)C=C)N=C2)ccc1F]

Step D: In a microwave tube was placed Step C product (770 mg, 1 mmol), 1-(2,2-dimethylbut-3-enyl)-4-(4,4,5,5-tetramethyl-1,3,2-dioxaborolan-2-yl)pyrazole (270 mg, 1 mmol), and palladium tetrakis(triphenylphosphine) (113 mg, 0.1 mmol). The tube was sealed and degassed with nitrogen (2-3 times). A mixture of toluene (7.5 mL)/ethanol (3 mL) was added, followed by 2 N aqueous sodium carbonate (3 mL, 6 mmol) was added. The mixture was stirred at 80° C. for 2 hours, cooled to room temperature and partitioned between water (20 mL) and ethyl acetate (20 mL). The separated organic layer, combined with two additional ethyl acetate extracts, was dried over sodium sulfate and concentrated. The residue was purified by silica gel chromatography to give a mixture (800 mg, 95.5%), which was separated by prep-HPLC. The first peak is assigned as Product 1(250 mg, 31%) and the second Peak is assigned as Product 2 (180 mg, 22%).

Product 1: MS (ESI): 858 m/z [M+H]⁺, retention time: 1.74 minutes, purity: >99% (214 nm) (LC-MS method 19).

Product 2: MS (ESI): 858 m/z [M+H]⁺, retention time: 1.76 minutes, purity: >99% (214 nm) (LC-MS method 19).

Ethyl 3-[3-[(8Z)-24,30-difluoro-10,10-dimethyl-21-(p-tolylsulfonyl)-26-oxa-5,12,13,21,33-pentazahexacyclo[25.3.1.12,5.112,15.017,25.018,22]tritriaconta-1(31),2(33),3,8,13,15(32),17,19,22,24,27,29-dodecaen-6-yl]phenyl]propanoate

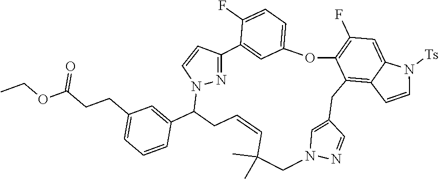 [SMILES: CCOC(=O)CCc1cccc(C2C/C=C\C(C)(C)Cn3cc(cn3)Cc3c(c(F)cc4c3ccn4S(=O)(=O)c3ccc(C)cc3)Oc3ccc(F)c(c3)-c3ccn2n3)c1]

Step E: To a solution of Product 1 of Step D product (40 mg, 0.05 mmol) in dry and degassed toluene (40 mL) was added Hoveyda-Grubb's (6 mg, 0.009 mmol). The mixture was heated to 115° C. for 60 hours under argon and concentrated. The residue was purified by silica gel column chromatography (petroleum ether ethyl acetate=11) to get the crude title compound (58 mg, crude) as a yellow oil. MS (ESI): 830 m/z [M+H]⁺, retention time: 2.50 minutes (LC-MS method 19).

Compound 84-3: 3-[3-[(8Z)-24,30-Difluoro-10,10-dimethyl-26-oxa-5,12,13,21,33-pentazahexacyclo[25.3.1.12,5.112,15.017,25.018,22]tritriaconta-1(31),2(33),3,8,13,15(32),17,19,22,24,27,29-dodecaen-6-yl]phenyl]propanoic acid

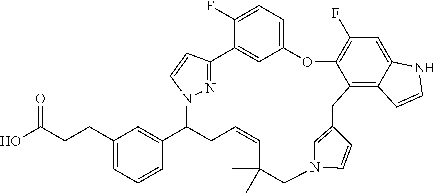 [SMILES: CC1(C)/C=C\CC(c2cccc(CCC(=O)O)c2)n2ccc(n2)-c2cc(ccc2F)Oc2c(F)cc3[nH]ccc3c2Cc2ccn(c2)C1]

Step F: To a stirred solution of Step E product (58 mg, crude) in methanol (3 mL) and water (0.5 mL) was added lithium hydroxide (84 mg, 3 mmol). The mixture was stirred at 30° C. for 30 hours and concentrated. Half of the residue was used for the next step hydrogenation, another half was purified by Prep-HPLC to give the title compound (2.0 mg) as a white solid. MS (ESI): 648.4 m/z [M+H]⁺, retention time: 2.03 minutes (LC-MS method 19).

Compound 84: 3-[3-(24,30-Difluoro-10,10-dimethyl-26-oxa-5,12,13,21,33-pentazahexacyclo[25.3.1.12,5.112,15.017,25.018,22]tritriaconta-1(31),2(33),3,13,15(32),17,19,22,24,27,29-undecaen-6-yl)phenyl]propanoic acid

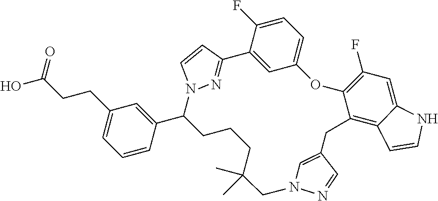 [SMILES: CC1(C)CCCC(c2cccc(CCC(=O)O)c2)n2ccc(n2)-c2cc(ccc2F)Oc2c(F)cc3[nH]ccc3c2Cc2cnn(c2)C1]

Step G: To a stirred solution of Step F product (18 mg, crude) in ethyl acetate (3 mL) was added palladium on carbon (10 mg). The mixture was stirred at 30° C. for 6 hours under hydrogen, then filtered through a pad of Celite. The residue was purified by Prep-HPLC to give the title compound (1.2 mg) as a white solid. MS (ESI): 650.3 m/z [M+H]⁺, retention time: 1.53 minutes (LC-MS method 19).

Ethyl 3-[3-[(9Z)-25,31-difluoro-11,11-dimethyl-22-(p-tolylsulfonyl)-27-oxa-5,6,13,14,22-pentazahexacyclo[26.3.1.113,16.02,6.018,26.019,23]tritriaconta-1(32),2,4,9,14,16(33),18,20,23,25,28,30-dodecaen-7-yl]phenyl]propanoate

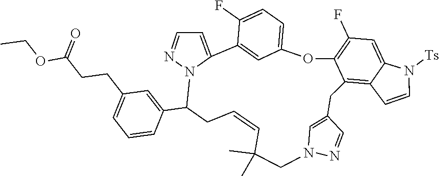 [SMILES: CCOC(=O)CCc1cccc(C2C/C=C\C(C)(C)Cn3cc(cn3)Cc3c(c(F)cc4c3ccn4S(=O)(=O)c3ccc(C)cc3)Oc3ccc(F)c(c3)-c3ccnn32)c1]

Step H: To a stirred solution of Product 2 of Step E product (300 mg, 0.35 mmol) in dry and degassed toluene (300 mL) was added Hoveyda-Grubb's catalyst (45 mg, 0.07 mmol). The mixture was heated to 115° C. for 60 hours under argon. After removal of the solvent, the residue was purified by silica gel column chromatography (petroleum ether\ethyl acetate=1\1) to get the title compound (83 mg, crude) as yellow oil. MS (ESI): 830.3 m/z [M+H]⁺, retention time: 2.67 minutes (LC-MS method 19).

3-[3-[(9Z)-25,31-Difluoro-11,11-dimethyl-27-oxa-5,6,13,14,22-pentazahexacyclo[26.3.1.113,16.02,6.018,26.019,23]tritriaconta-1(32),2,4,9,14,16(33),18,20,23,25,28,30-dodecaen-7-yl]phenyllpropanoic acid

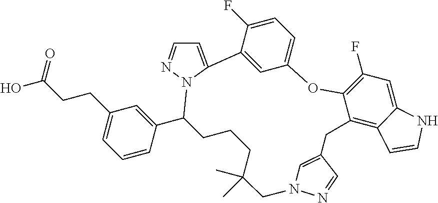 [SMILES: CC1(C)CCCC(c2cccc(CCC(=O)O)c2)n2nccc2-c2cc(ccc2F)Oc2c(F)cc3[nH]ccc3c2Cc2cnn(c2)C1]

Step I: To a solution of Step H product (83 mg, crude) in methanol (3 mL) and water (0.5 mL) was added lithium hydroxide (120 mg, 3 mmol). The mixture was stirred at 30° C. for 30 hours, and used directly for the next step without further purification. MS (ESI): 648.4 m/z [M+H]⁺, retention time: 2.24 minutes.

Compound 84-H4: 3-[3-(25,31-Difluoro-11,11-dimethyl-27-oxa-5,6,13,14,22-pentazahexacyclo[26.3.1.113,16.02,6.018,26.019,23]tritriaconta-1(32),2,4,14,16(33),18,20,23,25,28,30-undecaen-7-yl)phenyl]propanoic acid

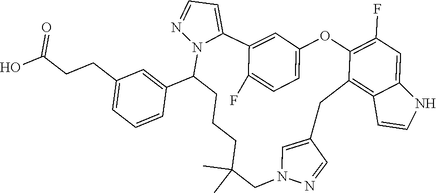 [SMILES: CC1(C)CCCC(c2cccc(CCC(=O)O)c2)n2nccc2-c2cc(ccc2F)Oc2c(F)cc3[nH]ccc3c2Cc2cnn(c2)C1]

Step J: To a solution of Step I product (83 mg, crude) in ethyl acetate (8 mL) was added palladium on carbon (50 mg). The mixture was stirred at 30° C. for 6 hours under hydrogen, then filtered through a pad of Celite. The filtrate was concentrated. The residue was purified by Prep-HPLC to give the title compound (12.1 mg, 5.3% of 3 steps) as a white solid. MS (ESI): 650.3 m/z [M+H]⁺, retention time: 1.53 minutes. ¹H NMR (400 MHz, CDCl₃): δ 8.57 (s, 1H), 7.47 (s, 1H). 7.43 (dd, J=6.0, 3.2 Hz, 1H), 7.35 (d, J=1.6 Hz, 1H), 7.21-7.16 (m, 2H), 7.14-7.04 (m, 4H), 6.97-6.95 (m, 2H), 6.71-6.68 (m, 2H), 6.24 (s, 1H), 5.04-4.98 (m, 1H), 4.27 (d, J=16.0 Hz, 1H), 3.97 (d, J=16.0 Hz, 1H), 3.65 (s, 2H), 2.91 (t, J=7.6 Hz, 2H), 2.64 (t, J=7.6 Hz, 2H), 2.17-2.06 (m, 1H), 1.68-1.59 (m, 1H), 1.18-0.84 (m, 4H), 0.79 (s, 3H), 0.74 (s, 3H) ppm.

Example 86. Compound 86. 3-[3-(24,30-Difluoro-11,11-dimethyl-26,32-dioxa-5,14,21,33-tetrazahexacyclo[25.3.1.12,5.112,15.017,25.018,22]tritriaconta-1(31),2(33),3,12,14,17,19,22,24,27,29-undecaen-6-yl)phenyl]propanoic acid

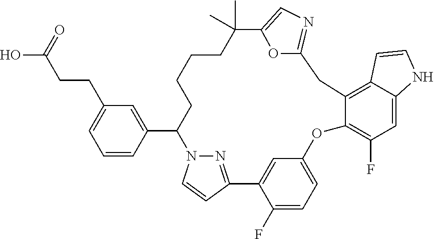 [SMILES: CC1(C)CCCCC(c2cccc(CCC(=O)O)c2)n2ccc(n2)-c2cc(ccc2F)Oc2c(F)cc3[nH]ccc3c2CC2=NC=C1O2]

Ethyl 2-(5-(3-(1-(1-(3-bromophenyl)-7-((tert-butyldimethylsilyl)oxy)-6,6-dimethylheptyl)-1H-pyrazol-3-yl)-4-fluorophenoxy)-6-fluoro-1-tosyl-1H-indol-4-yl)acetate

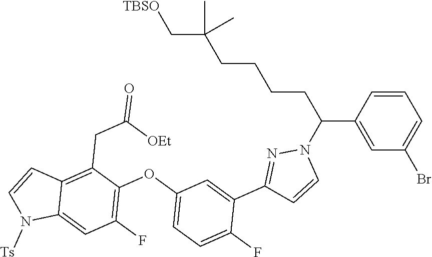 [SMILES: CCOC(=O)Cc1c(Oc2ccc(F)c(-c3ccn(C(CCCCC(C)(C)CO[Si](C)(C)C(C)(C)C)c4cccc(Br)c4)n3)c2)c(F)cc2c1ccn2S(=O)(=O)c1ccc(C)cc1]

Step A: To a stirred and cooled (0° C.) solution of ethyl 2-(6-fluoro-5-(4-fluoro-3-(1H-pyrazol-5-yl)phenoxy)-1-tosyl-1H-indol-4-yl)acetate (Intermediate 78, 2.4 g, 4.35 mmol) in dimethyl formamide (30 mL) were added ((7-bromo-7-(3-bromophenyl)-2,2-dimethylheptyl)oxy)(tert-butyl)dimethylsilane (Intermediate 59-2, 3.21 g, 6.53 mmol) and cesium carbonate (4.25 g, 13.1 mmol). The mixture was stirred at room temperature for 16 hours, quenched with 20 mL of water and extracted with ethyl acetate (50 mL×2). The combined organic phases were dried over sodium sulfate and concentrated. The residue was purified by silica gel chromatography (petroleum ether ethyl acetate=3:1) to give the title compound (2.6 g, 62.0%) as a white solid. MS (ESI): 962,964 m/z [M+H]⁺, retention time: 4.42 minutes, purity: 97% (214 nm) (LC-MS method 20).

2-(5-(3-(1-(1-(3-Bromophenyl)-7-((tert-butyldimethylsilyl)oxy)-6,6-dimethylheptyl)-1H-pyrazol-3-yl)-4-fluorophenoxy)-6-fluoro-1-tosyl-1H-indol-4-yl)acetic acid

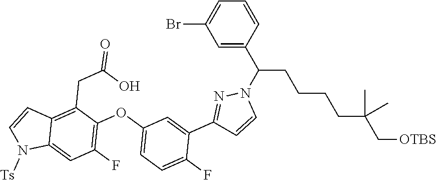 [SMILES: Cc1ccc(S(=O)(=O)n2ccc3c(CC(=O)O)c(Oc4ccc(F)c(-c5ccn(C(CCCCC(C)(C)CO[Si](C)(C)C(C)(C)C)c6cccc(Br)c6)n5)c4)c(F)cc32)cc1]

Step B: To a stirred solution of Step A product (3.4 g, 3.53 mmol) in tetrahydrofuran (20 mL), ethanol (20 mL) and water (20 mL) was added lithium hydroxide monohydrate (1.48 g, 35.3 mmol). The reaction was stirred at 40° C. for 16 hours, cooled to room temperature and quenched with 20 mL of water. The mixture was acidified with 1 N hydrochloric acid (pH to 5) and extracted with ethyl acetate (3×20 mL). The combined organic layers were dried over sodium sulfate and concentrated. The crude title compound (3.2 g, 97% yield) was obtained as a yellow oil. It was used for the next step without further purification. MS (ESI): 934, 936 m/z [M+H]⁺, retention time: 3.94 minutes, purity: 95% (214 nm) (LC-MS method 20).

(E)-2-(5-(3-(1-(7-((Tert-Butyldimethylsilyl)oxy)-1-(3-(3-ethoxy-3-oxoprop-1-en-1-yl)phenyl)-6,6-dimethylheptyl)-1H-pyrazol-3-yl)-4-fluorophenoxy)-6-fluoro-1-tosyl-1H-indol-4-yl)acetic acid

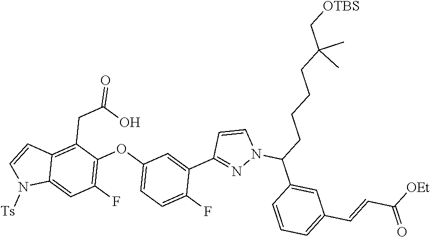 [SMILES: CCOC(=O)/C=C/c1cccc(C(CCCCC(C)(C)CO[Si](C)(C)C(C)(C)C)n2ccc(-c3cc(Oc4c(F)cc5c(ccn5S(=O)(=O)c5ccc(C)cc5)c4CC(=O)O)ccc3F)n2)c1]

Step C: To a stirred and degassed solution of Step B product (3.2 g, 3.42 mmol) in N,N-dimethylformamide (15 ml) were added ethyl acrylate (1.03 g, 10.3 mmol), tri(o-tolyl)phosphine (625 mg, 2.05 mmol), palladium (II) acetate (231 mg, 0.103 mmol) and triethylamine (1.73 g, 17.1 mmol). The reaction was stirred at 110° C. for 16 hours, cooled to room temperature, and quenched with 1 N hydrochloric acid (10 ml). The mixture was extracted with ethyl acetate (3×40 ml). The combined organic phase was dried over sodium sulfate and concentrated. The residue was purified by silica gel column chromatography (eluting with dichloromethane/methanol=40:1) to give the title compound (2.6 g, 80%) as a yellow oil. MS (ESI): 954 m/z [M+H]⁺, retention time: 3.89 minutes, purity: 90% (214 nm) (LC-MS method 10).

2-(5-(3-(1-(7-((Tert-Butyldimethylsilyl)oxy)-1-(3-(3-ethoxy-3-oxopropyl)phenyl)-6,6-dimethylheptyl)-1H-pyrazol-3-yl)-4-fluorophenoxy)-6-fluoro-1-tosyl-1H-indol-4-yl)acetic acid

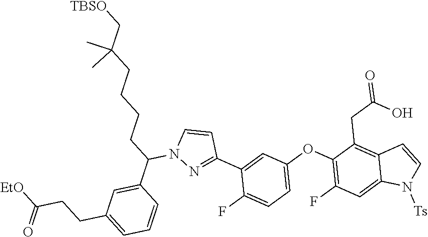 [SMILES: CCOC(=O)CCc1cccc(C(CCCCC(C)(C)CO[Si](C)(C)C(C)(C)C)n2ccc(-c3cc(Oc4c(F)cc5c(ccn5S(=O)(=O)c5ccc(C)cc5)c4CC(=O)O)ccc3F)n2)c1]

Step D: To a stirred solution of Step C product (2.6 g, 2.72 mmol) in ethyl acetate (20 mL) was added palladium on carbon (10%, 50% wet) (0.5 g). The mixture was stirred at room temperature for 5 hours under hydrogen. The mixture was filtered through a pad of Celite. The filtrate was concentrated to give the crude title compound (2.4 g, 92% yield) as yellow oil, which was used for the next step without further purification. MS (ESI): 956 m/z [M+H]⁺, retention time: 3.86 minutes, purity: 90% (214 nm) (LC-MS method 10).

Ethyl 3-(3-(1-(3-(5-((4-(2-(benzyloxy)-2-oxoethyl)-6-fluoro-1-tosyl-1H-indol-5-yl)oxy)-2-fluorophenyl)-1H-pyrazol-1-yl)-7-((tert-butyldimethylsilyl)oxy)-6,6-dimethylheptyl)phenyl)-propanoate

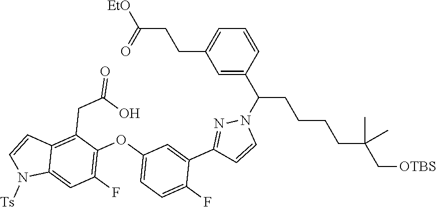 [SMILES: CCOC(=O)CCc1cccc(C(CCCCC(C)(C)CO[Si](C)(C)C(C)(C)C)n2ccc(-c3cc(Oc4c(F)cc5c(ccn5S(=O)(=O)c5ccc(C)cc5)c4CC(=O)O)ccc3F)n2)c1]

Step E: To a solution of Step D product (2.4 g, 2.51 mmol) in acetone (20 mL) were added potassium carbonate (0.52 g, 3.76 mmol) and benzyl bromide (0.515 g, 3.01 mmol). The mixture was stirred at 40° C. for one hour and concentrated. The residue was redissolved in dichloromethane (100 mL) and filtered. The filtrate was concentrated. The crude product was purified by silica gel column chromatography (eluting with petroleum ether/ethyl acetate=20:1) to give the title compound (2.3 g, 88%) as a yellow solid. MS (ESI): 1046 m/z [M+H]⁺, retention time: 4.54 minutes, purity: >99% (214 nm) (LC-MS method 2).

Ethyl 3-(3-(1-(3-(5-((4-(2-(benzyloxy)-2-oxoethyl)-6-fluoro-1-tosyl-1H-indol-5-yl)oxy)-2-fluorophenyl)-1H-pyrazol-1-yl)-7-hydroxy-6,6-dimethylheptyl)phenyl)propanoate

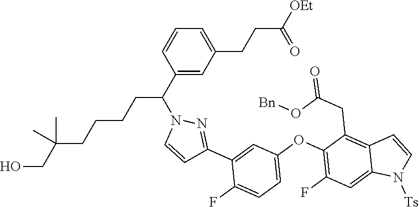 [SMILES: CCOC(=O)CCc1cccc(C(CCCCC(C)(C)CO)n2ccc(-c3cc(Oc4c(F)cc5c(ccn5S(=O)(=O)c5ccc(C)cc5)c4CC(=O)OCc4ccccc4)ccc3F)n2)c1]

Step F: To a solution of Step D product (2.2 g, 2.10 mmol) in tetrahydrofuran (20 mL) was added hydrochloric acid (3 N in water) (22 mL). The reaction was stirred at room temperature for 2 days and concentrated. The aqueous residue was neutralized to pH ˜7 using saturated sodium bicarbonate and extracted with ethyl acetate (3×50 mL). The combined organic phases were dried over sodium sulfate and concentrated. The crude title compound (1.5 g, 77%) was obtained as a yellow oil, which was used for next step without further purification. MS (ESI): 932 m/z [M+H]⁺, retention time: 2.52 minutes, purity: 95% (214 nm) (LC-MS method 15).

Ethyl 3-(3-(1-(3-(5-((4-(2-(benzyloxy)-2-oxoethyl)-6-fluoro-1-tosyl-1H-indol-5-yl)oxy)-2-fluorophenyl)-1H-pyrazol-1-yl)-6,6-dimethyl-7-oxoheptyl)phenyl)propanoate

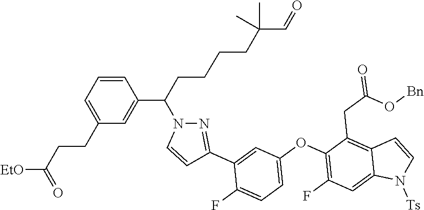 [SMILES: CCOC(=O)CCc1cccc(C(CCCCC(C)(C)C=O)n2ccc(-c3cc(Oc4c(F)cc5c(ccn5S(=O)(=O)c5ccc(C)cc5)c4CC(=O)OCc4ccccc4)ccc3F)n2)c1]

Step G: To a stirred solution of Step F product (1.20 g, 1.29 mmol) in dimethyl sulfoxide (10 mL) was added stabilized iodoxybenzoic acid (SIBX (46%), 1.08 g, 3.86 mmol). The mixture was stirred at 40° C. for one hour, cooled to room temperature and quenched with saturated sodium bicarbonate (15 mL) dropwise. The formed solid was collected by filtration and air-dried to give the title compound (1.2 g, 100%) as yellow solid, which was used for the next step without further purification. MS (ESI): 930 m/z [M+H]⁺, retention time: 2.51 minutes, purity: 51% (214 nm) (LC-MS method 15).

Ethyl 3-(3-(1-(3-(5-((4-(2-(benzyloxy)-2-oxoethyl)-6-fluoro-1-tosyl-1H-indol-5-yl)oxy)-2-fluorophenyl)-1H-pyrazol-1-yl)-7-hydroxy-6,6-dimethyl-8-nitrooctyl)phenyl)propanoate

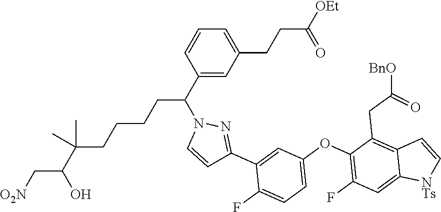 [SMILES: CCOC(=O)CCc1cccc(C(CCCCC(C)(C)C(O)C[N+](=O)[O-])n2ccc(-c3cc(Oc4c(F)cc5c(ccn5S(=O)(=O)c5ccc(C)cc5)c4CC(=O)OCc4ccccc4)ccc3F)n2)c1]

Step H: A mixture of Step G product (1.2 g, 1.29 mmol) and nitromethane (1.4 mL) and triethylamine (1.4 mL) was stirred at 40° C. for 16 hours and concentrated. The residue was purified by column chromatography (eluting with petroleum ether/ethyl acetate=2/1) to give the title compound (0.6 g, 47%) as a yellow oil. MS (ESI): 991 m/z [M+H]⁺, retention time: 2.39 minutes, purity: 99% (214 nm) (LC-MS method 15).

2-(5-(3-(1-(8-Amino-1-(3-(3-ethoxy-3-oxopropyl)phenyl)-7-hydroxy-6,6-dimethyloctyl)-1H-pyrazol-3-yl)-4-fluorophenoxy)-6-fluoro-1-tosyl-1H-indol-4-yl)acetic acid

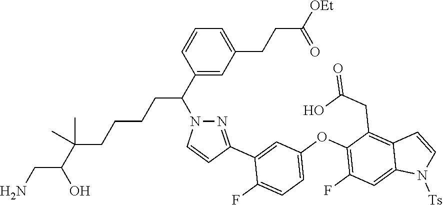 [SMILES: CCOC(=O)CCc1cccc(C(CCCCC(C)(C)C(O)CN)n2ccc(-c3cc(Oc4c(F)cc5c(ccn5S(=O)(=O)c5ccc(C)cc5)c4CC(=O)O)ccc3F)n2)c1]

Step I: To a solution of Step H product (0.60 g, 0.212 mmol) in tetrahydrofuran (10 mL) was added Raney Ni (200 mg). The mixture was stirred at room temperature under hydrogen for 3 hours. The mixture was filtered through a pad of Celite. The filtrate was concentrated. The residue was dissolved in ethyl acetate (10 mL), added Pd/C (10%, 50% wet, 200 mg). The mixture was stirred at room temperature under hydrogen for 16 hours then filtered through a pad of Celite. The filtrate was concentrated to give the crude title compound (0.18 g, 34%) as a yellow solid, which was used for the next step without further purification. MS (ESI): 871 m/z [M+H]⁺, retention time: 2.05 minutes, purity: 91% (214 nm) (LC-MS method 15).

Example 87. Compound 87. 3-[3-(24,30-Difluoro-11,11-dimethyl-26,32-dioxa-5,14,21,33-tetrazahexacyclo[25.3.1.12,5.112,15.017,25.018,22]tritriaconta-1(31),2(33),3,12,14,17,19,22,24,27,29-undecaen-6-yl)phenyl]propanoic acid

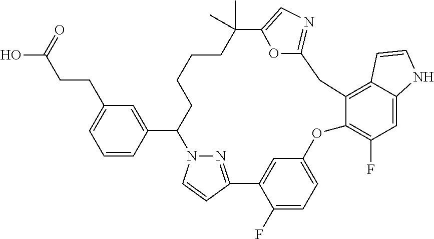 [SMILES: CC1(C)CCCCC(c2cccc(CCC(=O)O)c2)n2ccc(n2)-c2cc(ccc2F)Oc2c(F)cc3[nH]ccc3c2CC2=NC=C1O2]

Step J: Exchanging (E)-3-(5-(3-(5-(7-Amino-6-hydroxy-2-(3-iodophenyl)heptan-2-yl)-1H-imidazol-2-yl)-4-fluorophenoxy)-6-fluoro-1H-indol-4-yl)acrylic acid (Step C product of Example 21), with 2-(5-(3-(1-(8-Amino-1-(3-(3-ethoxy-3-oxopropyl)phenyl)-7-hydroxy-6,6-dimethyloctyl)-1H-pyrazol-3-yl)-4-fluorophenoxy)-6-fluoro-1-tosyl-1H-indol-4-yl)acetic acid (Step J product of this Example, 0.18 g, 0.207 mmol), the reaction procedure sequence (steps D, G, H and I) described for Example 21 was used to prepare the title compounds (43.7 mg). MS (ESI): 651 m/z [M+H]⁺, retention time: 2.19 minutes, purity: 97% (214 nm) (LC-MS method 20). ¹H NMR (400 MHz, CD₃OD) δ 7.61 (d, J=2.0 Hz, 1H), 7.46-7.42 (m, 1H), 7.34 (d, J=10.8 Hz, 1H), 7.31 (d, J=3.2 Hz, 1H), 7.25-7.20 (m, 2H), 7.14-7.07 (m, 3H), 6.94-6.89 (m, 1H), 6.66-6.64 (m, 1H), 6.54 (d, J=3.2 Hz, 1H), 6.50 (s, 1H), 5.21 (dd, J=11.6, 2.4 Hz, 1H), 4.33 (d, J=15.6 Hz, 1H), 4.26 (d, J=16.0 Hz, 1H), 2.91 (t, J=8.0 Hz, 2H), 2.59 (t, J=8.0 Hz, 2H), 2.55-2.49 (m, 1H), 1.87-1.83 (m, 1H), 1.46-1.24 (m, 4H), 1.14 (s, 3H), 1.11-1.07 (m, 1H), 0.99 (s, 3H), 0.91-0.86 (m, 1H) ppm.

Example 88. Compound 88A and Compound 88B. 3-[3-(25,31-Difluoro-12,12-dimethyl-27-oxa-5,15,16,22,34-pentazahexacyclo[26.3.1.12,5.113,16.018,26.019,23]tetratriaconta-1(32),2(34),3,13(33),14,18,20,23,25,28,30-undecaen-6-yl)phenyl]propanoic acid

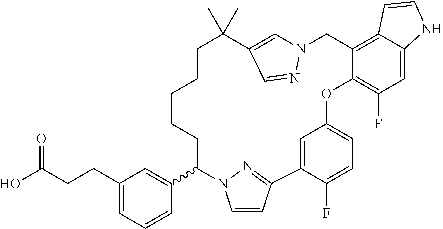 [SMILES: CC1(C)CCCCCC(c2cccc(CCC(=O)O)c2)n2ccc(n2)-c2cc(ccc2F)Oc2c(F)cc3[nH]ccc3c2Cn2cc1cn2]

Ethyl 3-(3-(1-(3-(2-fluoro-5-((6-fluoro-4-(hydroxymethyl)-1-tosyl-1H-indol-5-yl)oxy)phenyl)-1H-pyrazol-1-yl)pent-4-en-1-yl)phenyl)propanoate

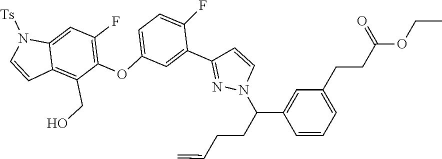 [SMILES: C=CCCC(c1cccc(CCC(=O)OCC)c1)n1ccc(-c2cc(Oc3c(F)cc4c(ccn4S(=O)(=O)c4ccc(C)cc4)c3CO)ccc2F)n1]

Step A: To a stirred solution of ethyl 3-(3-(1-bromopent-4-en-1-yl)phenyl)propanoate (Intermediate 59-3, 985 mg, 3.03 mmol) in dimethylformamide (10 mL) were added (6-fluoro-5-(4-fluoro-3-(1H-pyrazol-3-yl)phenoxy)-1-tosyl-1H-indol-4-yl)methanol (Intermediate 55-3, 1 g, 2.02 mmol) and cesium carbonate (1.32 g, 4.04 mmol). The solution was stirred at room temperature for 12 hours. The solvent was removed. The residue was re-dissolved in dichloromethane, washed with water, dried over magnesium sulfate, and concentrated. The crude product was purified by silica gel column chromatography (eluting with 20% ethyl acetate in petroleum ether) to give the title compound (900 mg, 60%). MS (ESI): 740 m/z [M+H]⁺, retention time: 1.72 minutes, purity: >99% (254 nm) (LC-MS method 18).

Ethyl 3-(3-(1-(3-(5-((4-(bromomethyl)-6-fluoro-1-tosyl-1H-indol-5-yl)oxy)-2-fluorophenyl)-1H-pyrazol-)-yl)pent-4-en-1-yl)phenyl)propanoate

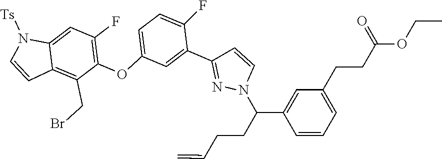 [SMILES: C=CCCC(c1cccc(CCC(=O)OCC)c1)n1ccc(-c2cc(Oc3c(F)cc4c(ccn4S(=O)(=O)c4ccc(C)cc4)c3CBr)ccc2F)n1]

Step B: To a stirred and cooled (0° C.) solution of Step A product (900 mg, 1.22 mmol) in dichloromethane (10 mL) were added phosphorus tribromide (659 mg, 2.43 mmol) and 2,6-lutidine (521 mg, 4.87 mmol). The solution was stirred at 0° C. for 1 hour, then diluted with dichloromethane (50 mL), washed with water (50 mL), dried over magnesium sulfate and concentrated. The residue was purified by silica gel column chromatography (eluting with 10% ethyl acetate in petroleum ether) to give the title compound (700 mg, 72%). MS (ESI): 802, 804 m/z [M+H]⁺, retention time: 1.88 minutes, purity: 95% (254 nm) (LC-MS method 18).

Ethyl 3-(3-(1-(3-(2-fluoro-5-((6-fluoro-4-((4-(2-methylpent-4-en-2-yl)-1H-pyrazol-1-yl)methyl)-1-tosyl-1H-indol-5-yl)oxy)phenyl)-1H-pyrazol-1-yl)pent-4-en-1-yl)phenyl)propanoate

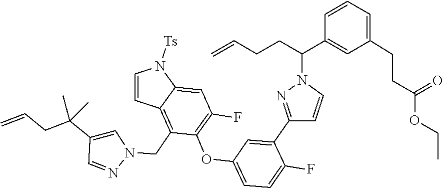 [SMILES: C=CCCC(c1cccc(CCC(=O)OCC)c1)n1ccc(-c2cc(Oc3c(F)cc4c(ccn4S(=O)(=O)c4ccc(C)cc4)c3Cn3cc(C(C)(C)CC=C)cn3)ccc2F)n1]

Step C: To a stirred solution of Step B product (700 mg, 0.872 mmol) in dimethylformamide (8 mL) were added 4-(1,1-dimethylbut-3-enyl)-1H-pyrazole (Intermediate 79, 170 mg, 1.13 mmol) in dimethylformamide (8 mL) and cesium carbonate (511 mg, 1.57 mmol). The solution was stirred at room temperature for 12 hours and concentrated. The residue was re-dissolved in dichloromethane (100 mL). The solution was washed with water (100 mL), dried over magnesium sulfate, and concentrated. The crude product was purified by silica gel column chromatography (eluting with 20% ethyl acetate in petroleum ether) to give the title compound (550 mg, 73%). MS (ESI): 872 m/z [M+H]⁺, retention time: 1.92 minutes, purity: 88% (254 nm) (LC-MS method 18).

Ethyl 3-[3-[(9E)-25,31-difluoro-12,12-dimethyl-22-(p-tolylsulfonyl)-27-oxa-5,15,16,22,34-pentazahexacyclo[26.3.1.12,5.113,16.018,26.019,23]tetratriaconta-1(32),2(34),3,9,13(33),14,18,20,23,25,28,30-dodecaen-6-yl]phenyl]propanoate

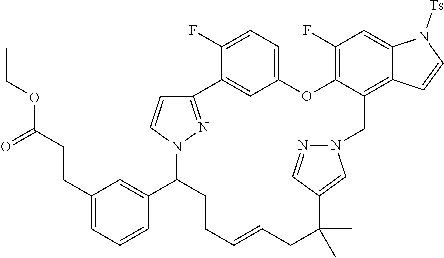 [SMILES: CCOC(=O)CCc1cccc(C2CC/C=C/CC(C)(C)c3cnn(c3)Cc3c(c(F)cc4c3ccn4S(=O)(=O)c3ccc(C)cc3)Oc3ccc(F)c(c3)-c3ccn2n3)c1]

Step D: To a solution of Step C product (550 mg, 0.631 mmol) in toluene (500 mL) was added (1,3-dimesitylimidazolidin-2-ylidene)(2-isopropoxybenzylidene)ruthenium(VI) chloride (Hoveyda-Grubb's 2^{nd} G catalyst, 79 mg, 0.126 mmol). The solution was stirred at 60° C. for 12 hours and concentrated. The residue was purified by silica gel column chromatography (eluting with 20% ethyl acetate in petroleum ether) to give the title compound (350 mg, 66%). MS (ESI): 844 m/z [M+H]⁺, retention time: 1.92 minutes, purity: 99% (254 nm) (LC-MS method 18).

Ethyl 3-[3-[(9E)-25,31-difluoro-12,12-dimethyl-27-oxa-5,15,16,22,34-pentazahexacyclo[26.3.1.12,5.113,16.018,26.019,23]tetratriaconta-1(32),2(34),3,9,13(33),14,18,20,23,25,28,30-dodecaen-6-yl]phenyl]propanoate

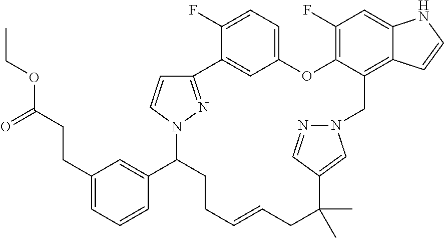 [SMILES: CCOC(=O)CCc1cccc(C2CC/C=C/CC(C)(C)c3cnn(c3)Cc3c(c(F)cc4[nH]ccc34)Oc3ccc(F)c(c3)-c3ccn2n3)c1]

Step E: A solution of Step D product (350 mg, 0.415 mmol) in tetra-n-butyl ammonium fluoride (1M in tetrahydrofuran, 6 mL) was stirred at room temperature for 4 hours. The mixture was partitioned between water (20 mL) and dichloromethane (20 mL). The separated organic layer was dried over magnesium sulfate and concentrated. The residue was purified by silica gel column chromatography (eluting with 30% ethyl acetate in petroleum ether) to give the title compound (250 mg, 89%). MS (ESI): 690 m/z [M+H]⁺, retention time: 1.71 minutes, purity: 81% (254 nm) (LC-MS method 18).

Ethyl 3-[3-(25,31-difluoro-12,12-dimethyl-27-oxa-5,15,16,22,34-pentazahexacyclo-[26.3.1.12,5.113,16.018,26.019,23]tetratriaconta-1(32),2(34),3,13(33),14,18,20,23,25,28,30-undecaen-6-yl)phenyl]propanoate

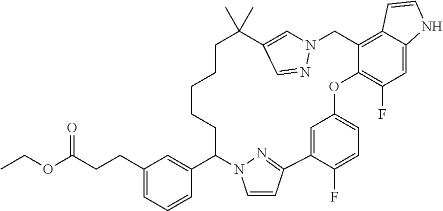 [SMILES: CCOC(=O)CCc1cccc(C2CCCCCC(C)(C)c3cnn(c3)Cc3c(c(F)cc4[nH]ccc34)Oc3ccc(F)c(c3)-c3ccn2n3)c1]

Step F: To a stirred solution of Step E product (200 mg, 0.296 mmol) in 5 mL of ethyl acetate was added palladium on carbon (10%, 50% wet, 94.5 mg). The reaction mixture was stirred at room temperature under hydrogen balloon for 24 hours, then filtered through a pad of Celite. The filtrate was concentrated. The crude product was purified by silica gel column chromatography (eluting with 25% ethyl acetate in petroleum ether) to give the title compound (120 mg, 60%). MS (ESI): 692 m/z [M+H]⁺, retention time: 2.68 minutes, purity: 72% (254 nm) (LC-MS method 18).

Enantiomers 1 and 2 of ethyl 3-[3-(25,31-difluoro-12,12-dimethyl-27-oxa-5,15,16,22,34-pentazahexacyclo-[26.3.1.12,5.113,16.018,26.019,23]tetratriaconta-1(32),2(34),3,13(33),14,18,20,23,25,28,30-undecaen-6-yl)phenyl]propanoate

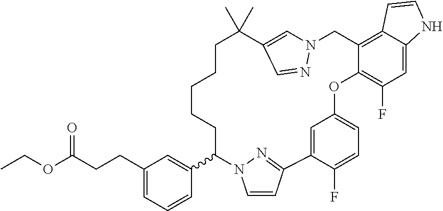 [SMILES: CCOC(=O)CCc1cccc(C2CCCCCC(C)(C)c3cnn(c3)Cc3c(c(F)cc4[nH]ccc34)Oc3ccc(F)c(c3)-c3ccn2n3)c1]

Step G: The Step F racemic product was subject to chiral prep-HPLC separation using Gilson-281 under the following condition: Column: OD 20*250 mm, 10 μm; Mobile Phase: n-Hexane (0.1% diethylamine): Ethanol (0.1% diethylamine)=95:5; Flow Rate: 40 mL/minutes; Run time per injection: 32 minutes; Injection: 0.7 mL; Sample solution: 160 mg in 15 mL methanol. The first eluent (40 mg, 33%) was named Enantiomer 1; The second eluent (40 mg, 33%) was named Enantiomer 2.

Enantiomer 1: MS (ESI): 692 m/z [M+H]⁺, retention time: 2.68 minutes, purity: 98% (254 nm) (LC-MS method 18).

Enantiomer 2: MS (ESI): 692 m/z [M+H]⁺, retention time: 2.68 minutes, purity: 97% (254 nm) (LC-MS method 18).

Compound 88A: Enantiomer 1 of 3-[3-(25,31-Difluoro-12,12-dimethyl-27-oxa-5,15,16,22,34-pentazahexacyclo[26.3.1.12,5.113,16.018,26.019,23]tetratriaconta-1(32),2(34),3,13(33),14,18,20,23,25,28,30-undecaen-6-yl)phenyl]propanoic acid

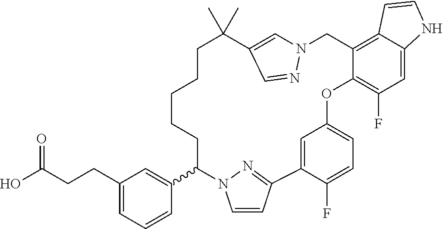 [SMILES: CC1(C)CCCCCC(c2cccc(CCC(=O)O)c2)n2ccc(n2)-c2cc(ccc2F)Oc2c(F)cc3[nH]ccc3c2Cn2cc1cn2]

Step H: To a stirred and cooled (0° C.) solution of Enantiomer 1 of Step G product (40 mg, 0.057 mmol) in 2 mL of tetrahydrofuran and 0.5 mL of water was added lithium hydroxide (10 mg, 0.24 mmol). The mixture was stirred at room temperature for 1 hour, then adjusted the pH to 5-6 with 2 N hydrochloric acid solution. The solution was diluted with ethyl acetate (50 mL). The mixture was washed with brine, dried over magnesium sulfate, and concentrated to provide the title compound (25 mg, 65%) as a white solid. MS (ESI): 664 m/z [M+H]⁺, retention time: 1.44 minutes, purity: 98% (254 nm) (LC-MS method 21). ¹H NMR (400 MHz, CD₃OD) δ 7.59 (d, J=2.4 Hz, 1H), 7.50-7.45 (m, 1H), 7.35-7.39 (m, 2H), 7.25 (s, 1H), 7.23-7.19 (m, 2H), 7.18-7.12 (m, 4H), 6.98-7.01 (m, 1H), 6.67-6.63 (m, 2H), 5.52 (s, 2H), 5.30-5.25 (m, 1H), 2.89 (t, J=7.6 Hz, 2H), 2.56 (t, J=7.6 Hz, 2H), 2.49-2.37 (m, 1H), 1.86-1.79 (m, 1H), 1.34-1.28 (m, 4H), 1.25-1.15 (m, 4H), 1.08 (s, 3H), 1.01 (s, 3H) ppm.

Compound 88B: Enantiomer 2 of 3-[3-(25,31-Difluoro-12,12-dimethyl-27-oxa-5,15,16,22,34-pentazahexacyclo[26.3.1.12,5.113,16.018,26.019,23]tetratriaconta-1(32),2(34),3,13(33),14,18,20,23,25,28,30-undecaen-6-yl)phenyl]propanoic acid

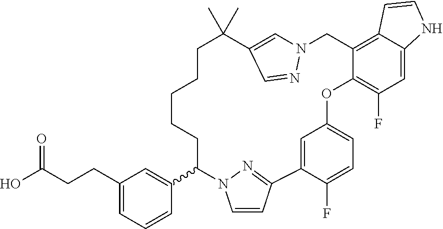 [SMILES: CC1(C)CCCCCC(c2cccc(CCC(=O)O)c2)n2ccc(n2)-c2cc(ccc2F)Oc2c(F)cc3[nH]ccc3c2Cn2cc1cn2]

Step I: The enantiomer 2 of Step G product (30 mg, 0.043 mmol) was subject to the identical reaction condition as described in Step H to afford the title compound (15 mg, 52%) as a white solid. MS (ESI): 664 m/z [M+H]⁺, retention time: 1.44 minutes, purity: >99% (254 nm) (LC-MS method 21). ¹H NMR (400 MHz, CD₃OD) δ 7.59 (d, J=2.4 Hz, 1H), 7.50-7.46 (m, 1H), 7.35-7.39 (m, 2H), 7.25 (s, 1H), 7.23-7.19 (m, 2H), 7.18-7.12 (m, 4H), 7.00-6.95 (m, 1H), 6.67-6.63 (m, 2H), 5.52 (s, 2H), 5.30-5.25 (m, 1H), 2.89 (t, J=7.6 Hz, 2H), 2.56 (t, J=7.6 Hz, 2H), 2.49-2.37 (m, 1H), 1.86-1.79 (m, 1H), 1.34-1.28 (m, 4H), 1.25-1.15 (m, 4H), 1.08 (s, 3H), 1.01 (s, 3H) ppm.

Example 89. Compound 89A and Compound 89B. Enantiomers 1 and 2 of 3-[3-(24,30-difluoro-11,11-dimethyl-26-oxa-5,14,15,21,33-pentazahexacyclo[25.3.1.12,5.112,15.017,25.018,22]-tritriaconta-1(31),2(33),3,12(32),13,17,19,22,24,27,29-undecaen-6-yl)phenyl]propanoic acid

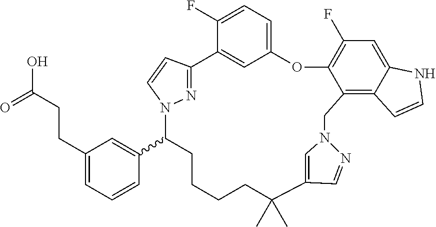 [SMILES: CC1(C)CCCCC(c2cccc(CCC(=O)O)c2)n2ccc(n2)-c2cc(ccc2F)Oc2c(F)cc3[nH]ccc3c2Cn2cc1cn2]

and

Compound 89C and Compound 89D. Enantiomers 1 and 2 of 3-[3-(25,31-difluoro-12,12-dimethyl-27-oxa-5,6,15,16,22-pentazahexacyclo-[26.3.1.113,16.02,6.018,26.019,23]tritriaconta-1(32),2,4,13(33),14,18,20,23,25,28,30-undecaen-7-yl)phenyl]propanoic acid

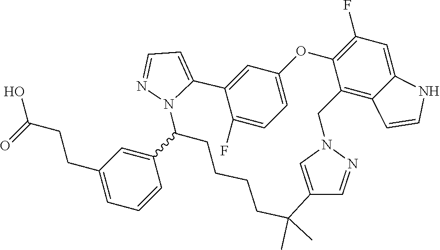 [SMILES: CC1(C)CCCCC(c2cccc(CCC(=O)O)c2)n2nccc2-c2cc(ccc2F)Oc2c(F)cc3[nH]ccc3c2Cn2cc1cn2]

Mixture of ethyl 3-(3-(1-(3-(5-((4-(((tert-butyldiphenylsilyl)oxy)methyl)-6-fluoro-1-tosyl-1H-indol-5-yl)oxy)-2-fluorophenyl)-1H-pyrazol-1-yl)but-3-en-1-yl)phenyl)propanoate and ethyl 3-(3-(1-(5-(5-((4-(((tert-butyldiphenylsilyl)oxy)methyl)-6-fluoro-1-tosyl-1H-indol-5-yl)oxy)-2-fluorophenyl)-1H-pyrazol-1-yl)but-3-en-1-yl)phenyl)propanoate

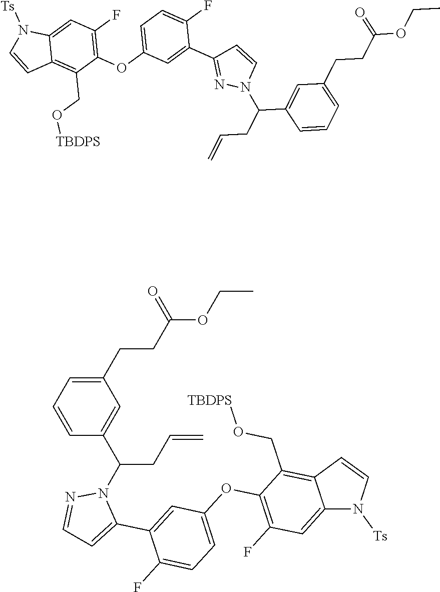

Step A: To a stirred solution of 4-(((tert-butyldiphenylsilyl)oxy)methyl)-6-fluoro-5-(4-fluoro-3-(1H-pyrazol-3-yl)phenoxy)-1-tosyl-1H-indole (Intermediate 81, 2.5 g, 3.41 mmol) and ethyl 3-(3-(1-hydroxybut-3-en-1-yl)phenyl)propanoate (Intermediate 75, 1.18 g, 4.77 mmol) in 1,4-dioxane (15 mL) was added cyanomethylenetributylphosphorane (2.06 g, 8.52 mmol). The solution was stirred under microwave at 150° C. for 40 minutes and concentrated. The residue was redissolved in dichloromethane (100 mL), washed with water, dried over magnesium sulfate and concentrated. The crude product was purified by silica gel column chromatography (eluting with 20% ethyl acetate in petroleum ether) to give the title compound (2.6 g, 80%) as an inseparable mixture. MS (ESI): 964 m/z [M+H]⁺, retention time: 1.97 minutes, purity: 95% (254 nm) (LC-MS method 18).

Mixture of ethyl 3-(3-(1-(3-(2-fluoro-5-((6-fluoro-4-(hydroxymethyl)-1-tosyl-1H-indol-5-yl)oxy)phenyl)-1H-pyrazol-1-yl)but-3-en-1-yl)phenyl)propanoate and ethyl 3-(3-(1-(5-(2-fluoro-5-((6-fluoro-4-(hydroxymethyl)-1-tosyl-1H-indol-5-yl)oxy)phenyl)-1H-pyrazol-1-yl)but-3-en-1-yl)phenyl)propanoate

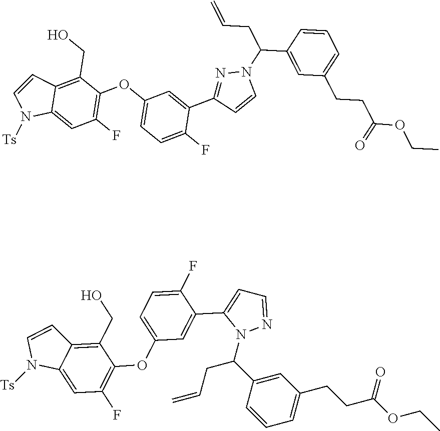 [SMILES: C=CCC(c1cccc(CCC(=O)OCC)c1)n1ccc(-c2cc(Oc3c(F)cc4c(ccn4S(=O)(=O)c4ccc(C)cc4)c3CO)ccc2F)n1 ; C=CCC(c1cccc(CCC(=O)OCC)c1)n1nccc1-c1cc(Oc2c(F)cc3c(ccn3S(=O)(=O)c3ccc(C)cc3)c2CO)ccc1F]

Step B: To a stirred solution of Step A product (2.6 g, 3.1 mmol) in tetrahydrofuran (25 mL) was added tetra-n-butyl ammonium fluoride (1.47 g, 4.67 mmol). The solution was stirred at room temperature for 4 hours and concentrated. The residue was redissolved in dichloromethane (50 mL), washed with water (50 mL), dried over magnesium sulfate and concentrated. The crude product was purified by silica gel column chromatography (eluting with 15% ethyl acetate in petroleum ether) to give the title compounds (2 g, 90%) as an inseparable mixture. MS (ESI): 726 m/z [M+H]⁺, retention time: 2.36 minutes, purity: 99% (254 nm) (LC-MS method).

Mixture of ethyl 3-(3-(1-(3-(5-((4-(bromomethyl)-6-fluoro-1-tosyl-1H-indol-5-yl)oxy)-2-fluorophenyl)-1H-pyrazol-1-yl)but-3-en-1-yl)phenyl)propanoate and ethyl 3-(3-(1-(5-(5-((4-(bromomethyl)-6-fluoro-1-tosyl-1H-indol-5-yl)oxy)-2-fluorophenyl)-1H-pyrazol-1-yl)but-3-en-1-yl)phenyl)propanoate

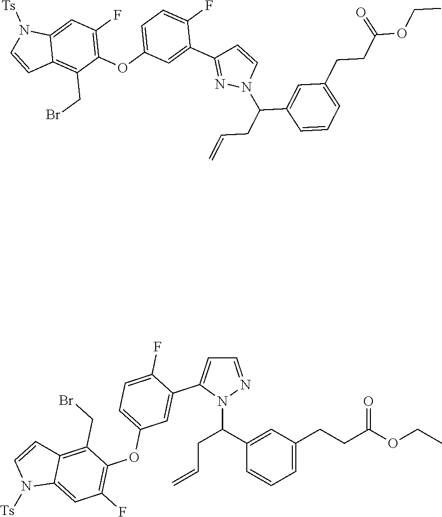 [SMILES: C=CCC(c1cccc(CCC(=O)OCC)c1)n1ccc(-c2cc(Oc3c(F)cc4c(ccn4S(=O)(=O)c4ccc(C)cc4)c3CBr)ccc2F)n1 ; C=CCC(c1cccc(CCC(=O)OCC)c1)n1nccc1-c1cc(Oc2c(F)cc3c(ccn3S(=O)(=O)c3ccc(C)cc3)c2CBr)ccc1F]

Step C: To a stirred and cooled (0° C.) solution of Step B product (2 g, 2.92 mmol) in dichloromethane (20 mL) were added 2,6-Lutidine (940 mg, 8.77 mmol) and phosphorus tribromide (1.58 mg, 5.85 mmol). The solution was stirred at room temperature for 12 hours, then diluted with dichloromethane (100 mL). The mixture was washed with water (50 mL), dried over magnesium sulfate and concentrated. The residue was purified by silica gel (eluting with 10% ethyl acetate in petroleum ether) to give the title compound (2 g, 87%) as an inseparable mixture. MS (ESI): 788, 790 m/z [M+H]⁺, retention time: 1.61 minutes, purity: 88% (254 nm) (LC-MS method).

Mixture of ethyl 3-(3-(1-(3-(2-fluoro-5-((6-fluoro-4-((4-(2-methylpent-4-en-2-yl)-1H-pyrazol-1-yl)methyl)-1-tosyl-1H-indol-5-yl)oxy)phenyl)-1H-pyrazol-1-yl)but-3-en-1-yl)phenyl)propanoate and ethyl 3-(3-(1-(5-(2-fluoro-5-((6-fluoro-4-((4-(2-methylpent-4-en-2-yl)-1H-pyrazol-1-yl)methyl)-1-tosyl-1H-indol-5-yl)oxy)phenyl)-1H-pyrazol-1-yl)but-3-en-1-yl)phenyl)propanoate

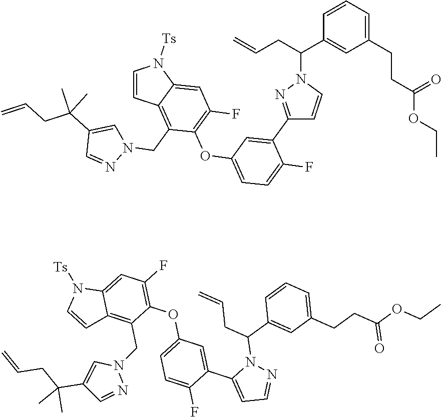 [SMILES: C=CCC(c1cccc(CCC(=O)OCC)c1)n1ccc(-c2cc(Oc3c(F)cc4c(ccn4S(=O)(=O)c4ccc(C)cc4)c3Cn3cc(C(C)(C)CC=C)cn3)ccc2F)n1 ; C=CCC(c1cccc(CCC(=O)OCC)c1)n1nccc1-c1cc(Oc2c(F)cc3c(ccn3S(=O)(=O)c3ccc(C)cc3)c2Cn2cc(C(C)(C)CC=C)cn2)ccc1F]

Step D: To a stirred solution of Step C product (2 g, 2.54 mmol) in dimethylformamide (20 mL) was added cesium carbonate (1.65 g, 5.07 mmol). The solution was stirred at room temperature for 12 hours, quenched with water (100 mL), and extracted with ethyl acetate (3×50 mL), the combined organic phases were washed with brine, dried over magnesium sulfate, and concentrated. The residue was purified by silica gel column (eluting with 20% ethyl acetate in petroleum ether) to give the title compound. (1.5 g, 70%). MS (ESI): 858 m/z [M+H]⁺, retention time: 1.69 minutes, purity: 95% (254 nm) (LC-MS method 25).

Compounds 89A, 89B. Enantiomers 1 and 2 of 3-[3-(24,30-difluoro-11,11-dimethyl-26-oxa-5,14,15,21,33-pentazahexacyclo[25.3.1.12,5.112,15.017,25.018,22]-tritriaconta-1(31),2(33),3,12(32),13,17,19,22,24,27,29-undecaen-6-yl)phenyl]propanoic acid

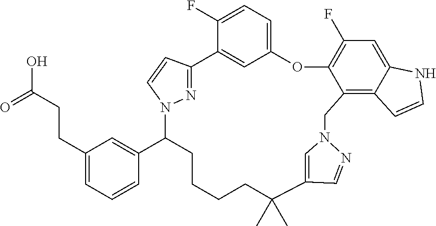 [SMILES: CC1(C)CCCCC(c2cccc(CCC(=O)O)c2)n2ccc(n2)-c2cc(ccc2F)Oc2c(F)cc3[nH]ccc3c2Cn2cc1cn2]

Compound 89C and 89D. Enantiomers 1 and 2 of 3-[3-(25,31-difluoro-12,12-dimethyl-27-oxa-5,6,15,16,22-pentazahexacyclo-[26.3.1.113,16.02,6.018,26.019,23]tritriaconta-1(32),2,4,13(33),14,18,20,23,25,28,30-undecaen-7-yl)phenyl]propanoic acid

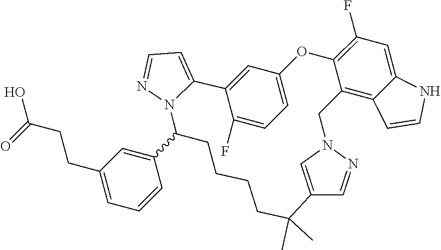 [SMILES: CC1(C)CCCCC(c2cccc(CCC(=O)O)c2)n2nccc2-c2cc(ccc2F)Oc2c(F)cc3[nH]ccc3c2Cn2cc1cn2]

Step E: Exchanging ethyl 3-(3-(1-(3-(2-fluoro-5-((6-fluoro-4-((4-(2-methylpent-4-en-2-yl)-1H-pyrazol-1-yl)methyl)-1-tosyl-1H-indol-5-yl)oxy)phenyl)-1H-pyrazol-1-yl)pent-4-en-1-yl)phenyl)propanoate (Step C product of Example 88A and 88B) with Step D product of this Example (2 g, 2.54 mmol), the reaction procedure sequence (Steps C to I) described for Example 88A and 88B) was used to prepare the title compounds. Two regio-isomers were separated using prep-HPLC, after hydrogenation, at the corresponding Step F of Example 88A and 88B). The first peak is designated to be regio-isomer 1 (130 mg); The second peak is regio-isomer 2 (120 mg).

The regio-isomer 1 (130 mg) was subject to chiral SFC separation using SFC-80 (Thar, Waters) under the following conditions: Column: WHELK 20*250 mm, 10 μm; Column temperature: 35° C.; Mobile phase: carbon dioxide/isopropyl alcohol (0.1% diethylamine as additive)=55/45; Flow rate: 80 g/minute; Back pressure: 100 bar; Detection wavelength: 214 nm; Cycle time: 5.5 minutes; Sample solution: 130 mg dissolved in 20 mL methanol; Injection volume: 1.0 mL. The first eluent (30 mg, 23%), Enantiomer 1, was further hydrolyzed to Compound 89A (20 mg, 70%); The second eluent (30 mg, 23%), Enantiomer 2, was further hydrolyzed to Compound 89B (20 mg, 70%), following the identical conditions as described in corresponding Step H of Example 88A and 88B).

The regio-isomer 2 (120 mg) was subject to chiral HPLC separation using instrument Gilson-281 under the following condition: Column: OD 20*250 mm, 10 μm; Mobile phase: methanol (0.1% formic acid): dichloromethane (0.1% formic acid)=90:10; Flow Rate: 50 mL/minute; Run time per injection: 17 minutes; Injection: 0.8 mL; Sample solution: 120 mg in 20 mL methanol. The first eluent (40 mg, 33%), Enantiomer 1, was further hydrolyzed to Compound 89C (30 mg, 78%; The second eluent (40 mg, 33%), Enantiomer 2, was further hydrolyzed to Compound 89D (30 mg, 78%).

Compound 89A: MS (ESI): 650 m/z [M+H]⁺, retention time: 2.29 minutes, purity: 98% (214 nm) (LC-MS method 22). ¹H NMR (400 MHz, CD₃OD) δ 7.65 (d, J=2.0 Hz, 1H), 7.39 (d, J=10.8 Hz, 1H), 7.34 (d, J=3.2 Hz, 1H), 7.32-7.29 (m, 1H), 7.25-7.04 (m, 8H), 6.69-6.66 (m, 1H), 6.58 (d, J=3.2 Hz, 1H), 5.65-5.52 (m, 2H), 5.20-5.18 (m, 1H), 2.88 (t, J=8.0 Hz, 2H), 2.53 (t, J=8.0 Hz, 2H), 2.33-2.29 (m, 1H), 1.87-1.79 (m, 1H), 1.31-1.27 (m, 4H), 1.07 (s, 3H), 1.01-0.96 (m, 1H), 0.94 (s, 3H), 0.76-0.71 (m, 1H) ppm.

Compound 89B: MS (ESI): 650 m/z [M+H]⁺, retention time: 2.30 minutes, purity: 98% (214 nm) (LC-MS method 22). ¹H NMR (400 MHz, CD₃OD) δ 7.65 (d, J=2.0 Hz, 1H), 7.39 (d, J=10.8 Hz, 1H), 7.34 (d, J=3.2 Hz, 1H), 7.33-7.29 (m, 1H), 7.25-7.04 (m, 8H), 6.69-6.66 (m, 1H), 6.58 (d, J=3.2 Hz, 1H), 5.66-5.51 (m, 2H), 5.20-5.18 (m, 1H), 2.88 (t, J=8.0 Hz, 2H), 2.53 (t, J=8.0 Hz, 2H), 2.33-2.29 (m, 1H), 1.87-1.79 (m, 1H), 1.31-1.27 (m, 4H), 1.07 (s, 3H), 1.01-0.96 (m, 1H), 0.94 (s, 3H), 0.76-0.71 (m, 1H) ppm.

Compound 89C: MS (ESI): 650 m/z [M+H]⁺, retention time: 2.25 minutes, purity: 99% (214 nm) (LC-MS method 22).1H NMR (400 MHz, CD₃OD) δ 7.61 (d, J=2.0 Hz, 1H), 7.46 (d, J=3.2 Hz, 1H), 7.26-7.15 (m, 5H), 7.07 (s, 1H), 6.98 (d, J=7.8 Hz, 1H), 6.91 (d, J=3.2 Hz, 1H), 6.55-6.45 (m, 2H), 6.39 (t, J=1.2 Hz, 1H), 6.29 (d, J=7.4 Hz, 1H), 5.73 (d, J=14.0 Hz, 1H), 5.63 (d, J=14.0 Hz, 1H), 4.78-4.74 (m, 1H), 2.81 (t, J=8.4 Hz, 2H), 2.46 (t, J=8.4 Hz, 2H), 2.18-2.09 (m, 1H), 1.84-1.78 (m, 1H), 1.37-1.34 (m, 2H), 1.23-1.14 (m, 1H), 1.12 (s, 3H), 1.00 (s, 3H), 0.95-0.86 (m, 1H), 0.68-0.66 (m, 1H), 0.47-0.45 (m, 1H) ppm.

Compound 89D: MS (ESI): 650 m/z [M+H]⁺, retention time: 2.25 minutes, purity: >99% (214 nm) (LC-MS method 22). ¹H NMR (400 MHz, CD₃OD) δ 7.61 (d, J=2.0 Hz, 1H), 7.46 (d, J=3.2 Hz, 1H), 7.26-7.15 (m, 5H), 7.07 (s, 1H), 6.98 (d, J=7.8 Hz, 1H), 6.91 (d, J=3.2 Hz, 1H), 6.56-6.45 (m, 2H), 6.39 (t, J=1.2 Hz, 1H), 6.29 (d, J=7.4 Hz, 1H), 5.73 (d, J=14.0 Hz, 1H), 5.63 (d, J=14.0 Hz, 1H), 4.78-4.73 (m, 1H), 2.81 (t, J=8.4 Hz, 2H), 2.46 (t, J=8.4 Hz, 2H), 2.18-2.09 (m, 1H), 1.84-1.78 (m, 1H), 1.37-1.34 (m, 2H), 1.23-1.14 (m, 1H), 1.12 (s, 3H), 1.00 (s, 3H), 0.95-0.86 (m, 1H), 0.68-0.66 (m, 1H), 0.47-0.45 (m, 1H) ppm.

Example 90. Compound 90A and Compound 90B. 3-[3-(24,30-Difluoro-10,10-dimethyl-9,26-dioxa-5,12,13,21,33-pentazahexacyclo[25.3.1.12,5.112,15.017,25.018,22]tritriaconta-1(31),2(33),3,13,15(32),17,19,22,24,27,29-undecaen-6-yl)phenyl]propanoic acid

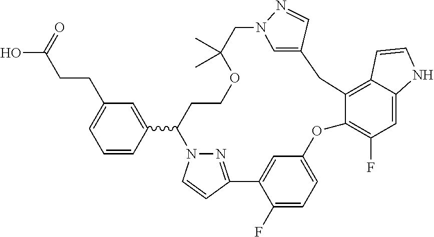 [SMILES: CC1(C)Cn2cc(cn2)Cc2c(c(F)cc3[nH]ccc23)Oc2ccc(F)c(c2)-c2ccn(n2)C(c2cccc(CCC(=O)O)c2)CCO1]

and

Example 91. Compound 91. 3-[3-(25,31-Difluoro-11,11-dimethyl-10,27-dioxa-5,6,13,14,22-pentazahexacyclo[26.3.1.113,16.02,6.018,26.019,23]tritriaconta-1(32),2,4,14,16(33),18,20,23,25,28,30-undecaen-7-yl)phenyl]propanoic acid

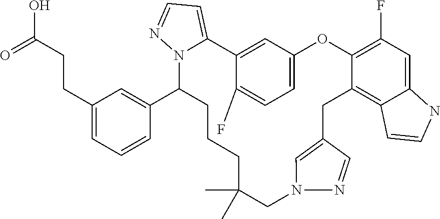 [SMILES: CC1(C)CCCC(c2cccc(CCC(=O)O)c2)n2nccc2-c2cc(ccc2F)Oc2c(F)cc3[nH]ccc3c2Cc2cnn(c2)C1]

Mixture of 5-(3-(1-(1-(3-bromophenyl)-3-((2-methylbut-3-en-2-yl)oxy)propyl)-1H-pyrazol-3-yl)-4-fluorophenoxy)-1-((4-bromophenyl)sulfonyl)-6-fluoro-4-((1-(tetrahydro-2H-pyran-2-yl)-1H-pyrazol-4-yl)methyl)-1H-indole and 5-(3-(1-(1-(3-bromophenyl)-3-((2-methylbut-3-en-2-yl)oxy)propyl)-1H-pyrazol-5-yl)-4-fluorophenoxy)-1-((4-bromophenyl)sulfonyl)-6-fluoro-4-((1-(tetrahydro-2H-pyran-2-yl)-1H-pyrazol-4-yl)methyl)-1H-indole

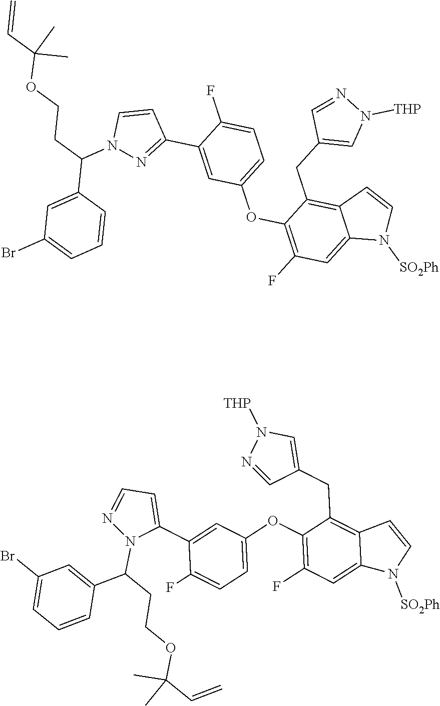

Step A: To a stirred solution of 6-fluoro-5-(4-fluoro-3-(1H-pyrazol-3-yl)phenoxy)-1-(phenylsulfonyl)-4-((1-(tetrahydro-2H-pyran-2-yl)-1H-pyrazol-4-yl)methyl)-1H-indole (Intermediate 73-2, 500 mg, 0.81 mmol) in 1,4-dioxane (12 mL) were added 1-(3-bromophenyl)-3-((2-methylbut-3-en-2-yl)oxy)propan-1-ol (Intermediate 77-1, 0.413 g, 1.38 mmol) and cyanomethylenetributylphosphorane (0.98 g, 4.06 mmol). The mixture was heated at 145° C. for 50 minutes in a microwave reactor. Three additional reactions with the same scale were run in parallel. The reaction mixtures were combined and concentrated. The residue was purified by automated silica gel column chromatography (80 g silica gel column, eluting with 0-60% ethyl acetate in petroleum ether) to give the title compound (2.08 g, 71%) as an inseparable mixture. MS (ESI): 896, 898 m/z [M+H]⁺, retention time: 1.71 minutes, purity: 98% (214 nm) (LC-MS method 21).

Mixture of 3-(3-(3-bromophenyl)-3-(3-(2-fluoro-5-((6-fluoro-1-(phenylsulfonyl)-4-((1-(tetrahydro-2H-pyran-2-yl)-1H-pyrazol-4-yl)methyl)-1H-indol-5-yl)oxy)phenyl)-1H-pyrazol-1-yl)propoxy)-3-methylbutane-1,2-diol and 3-(3-(3-bromophenyl)-3-(5-(2-fluoro-5-((6-fluoro-1-(phenylsulfonyl)-4-((1-(tetrahydro-2H-pyran-2-yl)-1H-pyrazol-4-yl)methyl)-1H-indol-5-yl)oxy)phenyl)-1H-pyrazol-1-yl)propoxy)-3-methylbutane-1,2-diol

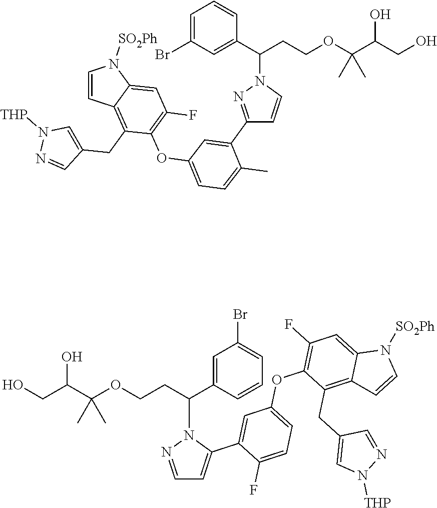

Step B: To a stirred solution of Step A product (2.08 g, 2.32 mmol) in acetone (114 mL) and water (25 mL) was added N-methylmorpholine N-oxide (4.8 M, 0.773 mL, 3.71 mmol) and osmium tetroxide (2 mg/mL, 11.4 mL). The mixture was stirred at room temperature for 16 hours, quenched with saturated sodium sulfite (50 mL), and evaporated to remove acetone. The aqueous residue was extracted with ethyl acetate (100 mL). The separated organic layer, combined with two additional ethyl acetate extracts (2×50 mL), was washed with brine, dried over sodium sulfate, and concentrated. The residue was purified by silica gel column chromatography (40 g silica gel column, eluting with 0-10% methanol in dichloromethane) to give the title compound (1.65 g, 76.4%) as an inseparable mixture. MS (ESI): 930, 932 m/z [M+H]⁺, retention time: 1.49 minutes, purity: 64% (214 nm) (LC-MS method 21).

Mixture of 2-(3-(3-bromophenyl)-3-(3-(2-fluoro-5-((6-fluoro-1-(phenylsulfonyl)-4-((1-(tetrahydro-2H-pyran-2-yl)-1H-pyrazol-4-yl)methyl)-1H-indol-5-yl)oxy)phenyl)-1H-pyrazol-1-yl)propoxy)-2-methylpropanal and 2-(3-(3-bromophenyl)-3-(5-(2-fluoro-5-((6-fluoro-1-(phenylsulfonyl)-4-((1-(tetrahydro-2H-pyran-2-yl)-1H-pyrazol-4-yl)methyl)-1H-indol-5-yl)oxy)phenyl)-1H-pyrazol-1-yl)propoxy)-2-methylpropanal

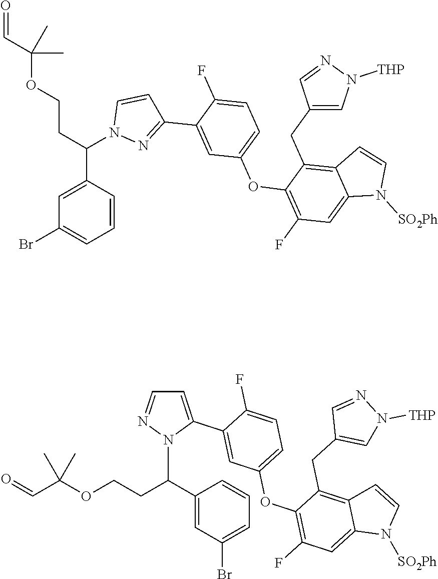

Step C: To a stirred solution of Step B product (1.65 g, 1.77 mmol) in acetone (60 mL) and water (20 mL) was added sodium periodate (1.14 g, 5.32 mmol). The reaction was stirred at room temperature for 16 hours and diluted with ethyl acetate (200 mL). The solution was washed with water, brine, dried over sodium sulfate, and concentrated to give the crude title compound (1.59 g, 100%, crude) as an inseparable mixture. MS (ESI): 898, 900 m/z [M+H]⁺, retention time: 1.63 minutes, purity: 56% (254 nm) (LC-MS method 21).

Mixture of 2-(3-(3-bromophenyl)-3-(3-(2-fluoro-5-((6-fluoro-1-(phenylsulfonyl)-4-((1-(tetrahydro-2H-pyran-2-yl)-1H-pyrazol-4-yl)methyl)-1H-indol-5-yl)oxy)phenyl)-1H-pyrazol-1-yl)propoxy)-2-methylpropan-1-ol and 2-(3-(3-bromophenyl)-3-(5-(2-fluoro-5-((6-fluoro-1-(phenylsulfonyl)-4-((1-(tetrahydro-2H-pyran-2-yl)-1H-pyrazol-4-yl)methyl)-1H-indol-5-yl)oxy)phenyl)-1H-pyrazol-1-yl)propoxy)-2-methylpropan-1-ol

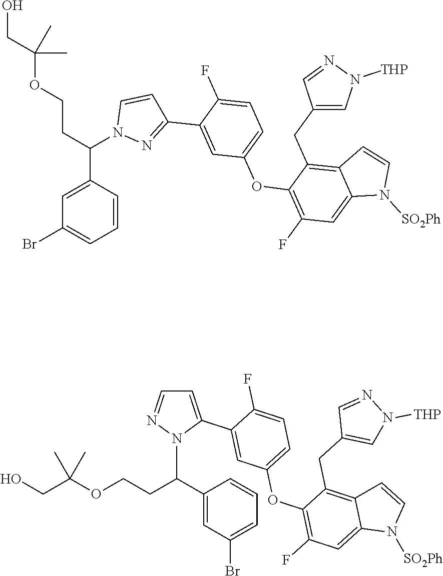

Step D: To a stirred solution of Step C product (1.59 g, 1.77 mmol) in methanol (30 mL) was added sodium borohydride (80.3 mg, 2.12 mmol). The reaction mixture was stirred at room temperature for one hour, then diluted with ethyl acetate. The solution was washed with water, dried over anhydrous magnesium sulfate, and concentrated to the crude title compound (1.59 g, 100%) as an inseparable solid. MS (ESI): 900, 902 m/z [M+H]⁺, retention time: 1.56 minutes, purity: 65% (214 nm) (LC-MS method 21).

Mixture of 2-(3-(3-(5-((4-((1H-pyrazol-4-yl)methyl)-6-fluoro-1-(phenylsulfonyl)-1H-indol-5-yl)oxy)-2-fluorophenyl)-1H-pyrazol-1-yl)-3-(3-bromophenyl)propoxy)-2-methylpropan-1-ol and 2-(3-(5-(5-((4-((1H-pyrazol-4-yl)methyl)-6-fluoro-1-(phenylsulfonyl)-1H-indol-5-yl)oxy)-2-Fluorophenyl)-1H-pyrazol-1-yl)-3-(3-bromophenyl)propoxy)-2-methylpropan-1-ol

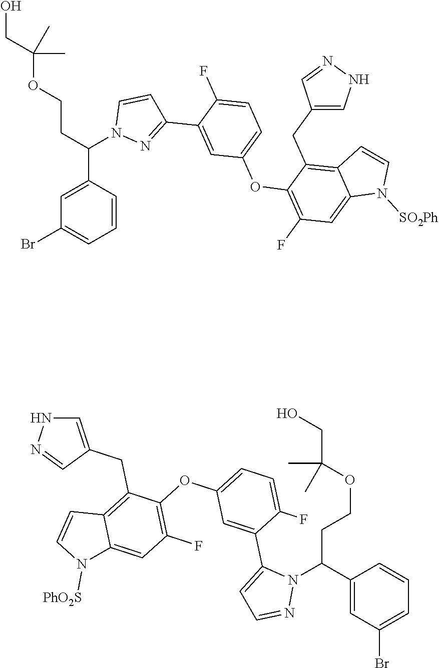

Step E: To a stirred solution of Step D product (1.59 g, 1.77 mmol) in methanol (40 mL) was added p-toluene-sulfonic acid (2.22 g, 8.83 mmol). The reaction mixture was stirred at 60° C. for 3 hours and concentrated. The residue was dissolved in ethyl acetate (50 mL), washed with water, brine, dried over anhydrous sodium sulfate, and concentrated. The crude product was purified by automated flash chromatography (40 g silica gel column, eluting with 0-100% ethyl acetate in petroleum ether) to give the title compound (1.01 g, 70%) as an inseparable solid. MS (ESI): 816, 818 m/z [M+H]⁺, retention time: 1.44 minutes, purity: 72% (214 nm) (LC-MS method 21).

Mixture of 21-(benzenesulfonyl)-6-(3-bromophenyl)-24,30-difluoro-10,10-dimethyl-9,26-dioxa-5,12,13,21,33-pentazahexacyclo[25.3.1.12,5.112,15.017,25.018,22]tritriaconta-1(31),2(33),3,13,15(32),17,19,22,24,27,29-undecaene and 22-(benzenesulfonyl)-7-(3-bromophenyl)-25,31-difluoro-11,11-dimethyl-10,27-dioxa-5,6,13,14,22-pentazahexacyclo[26.3.1.113,16.02,6.018,26.019,23]tritriaconta-1(32),2,4,14,16(33),18,20,23,25,28,30-undecaene

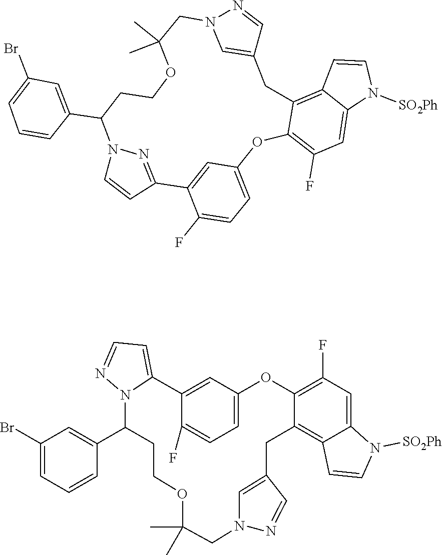

Step F: To a stirred solution of Step E product (0.3 g, 0.37 mmol) in 1,4-dioxane (15 mL) was added cyanomethylenetributylphosphorane (CMBP, 0.443 g, 1.84 mmol). The mixture was heated at 145° C. for 50 minutes in a microwave reactor. Two more same scale reactions were run in parallel. The reaction mixtures were combined and concentrated. The residue was purified by automated silica gel column chromatography (40 g silica gel column, eluting with 0-80% ethyl acetate in petroleum ether) to give the title compound (550 mg, 55%) as an inseparable solid. MS (ESI): 798, 800 m/z [M+H]⁺, retention time: 1.66 minutes, purity: 40% (214 nm) (LC-MS method 21).

Mixture of 6-(3-bromophenyl)-24,30-difluoro-10,10-dimethyl-9,26-dioxa-5,12,13,21,33-pentazahexacyclo[25.3.1.12,5.112,15.017,25.018,22]tritriaconta-1(31),2(33),3,13,15(32),17,19,22,24,27,29-undecaene and 7-(3-bromophenyl)-25,31-difluoro-11,11-dimethyl-10,27-dioxa-5,6,13,14,22-pentazahexacyclo[26.3.1.113,16.02,6.018,26.019,231-tritriaconta-1(32),2,4,14,16(33),18,20,23,25,28,30-undecaene

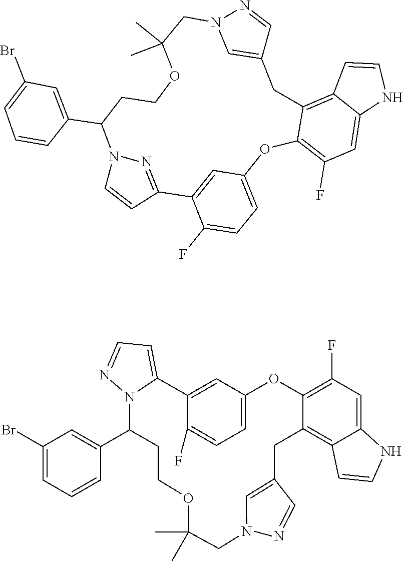 [SMILES: CC1(C)Cn2cc(cn2)Cc2c(c(F)cc3[nH]ccc23)Oc2ccc(F)c(c2)-c2ccn(n2)C(c2cccc(Br)c2)CCO1 ; CC1(C)Cn2cc(cn2)Cc2c(c(F)cc3[nH]ccc23)Oc2ccc(F)c(c2)-c2ccnn2C(c2cccc(Br)c2)CCO1]

Step G: A mixture of Step F product (550 mg, 0.69 mmol) and tetrabutylammonium fluoride (1 M in tetrahydrofuran) (30 mL) was stirred at 40° C. for 3 hours. The reaction mixture was diluted with ethyl acetate (100 mL), washed with water, brine, dried over sodium sulfate, and concentrated. The residue was purified by automated silica gel column chromatography (40 g silica gel column, eluting with 0-80% ethyl acetate in petroleum ether) to give the title compounds (0.38 g, 84%) as an inseparable solid. MS (ESI): 658, 660 m/z [M+H]⁺, retention time: 2.01 minutes, purity: 47% (214 nm) (LC-MS method 23).

Compound 90A, 90B. 3-[3-(24,30-Difluoro-10,10-dimethyl-9,26-dioxa-5,12,13,21,33-pentazahexacyclo[25.3.1.12,5.112,15.017,25.018,22]tritriaconta-1(31),2(33),3,13,15(32),17,19,22,24,27,29-undecaen-6-yl)phenyl]propanoic acid

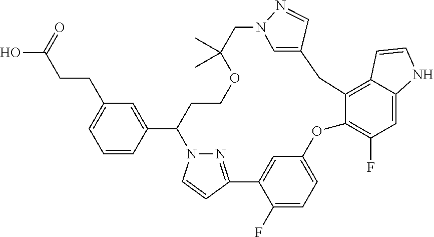 [SMILES: CC1(C)Cn2cc(cn2)Cc2c(c(F)cc3[nH]ccc23)Oc2ccc(F)c(c2)-c2ccn(n2)C(c2cccc(CCC(=O)O)c2)CCO1]

Compound 91. 3-[3-(25,31-Difluoro-11,11-dimethyl-10,27-dioxa-5,6,13,14,22-pentazahexacyclo[26.3.1.113,16.02,6.018,26.019,23]tritriaconta-1(32),2,4,14,16(33),18,20,23,25,28,30-undecaen-7-yl)phenyl]propanoic acid

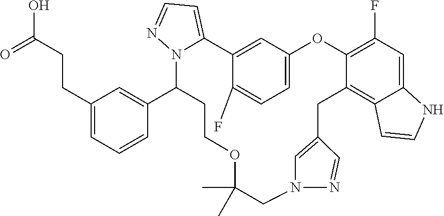 [SMILES: CC1(C)Cn2cc(cn2)Cc2c(c(F)cc3[nH]ccc23)Oc2ccc(F)c(c2)-c2ccnn2C(c2cccc(CCC(=O)O)c2)CCO1]

Step H: Exchanging 23,29-difluoro-6-(3-iodophenyl)-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaene (Step B product of Example 2) with Step F product of this example (0.28 g, 0.43 mmol), the reaction procedure sequence (Steps C, D and E) described for Example 2 was used to prepare the title compounds. The two regio-isomeric acids were separated using prep-HPLC at the corresponding Step E of Example 2. The first eluent is regio-isomer 1 (102 mg), and was determined by NOESY as Compound 91. The second eluent, regio-isomer 2 (106 mg), was subject to chiral SFC separation using SFC-80 (Thar, Waters) under the following condition: Flow rate: 80 g/min; Back pressure: 100 bar; Detection wavelength: 214 nm; Cycle time:4.5 minutes; Sample solution:106 mg dissolved in 15 mL methanol; Injection volume:4 mL The first eluent, Enantiomer 1, is designated as Compound 90A (44.1 mg, 42%); The second eluent, Enantiomer 2, was designated as Compound 90B (43.2 mg, 41%). Compound 91: MS (ESI): 652 m/z [M+H]⁺, retention time: 1.86 minutes, purity: 99% (214 nm) (LC-MS method 23). ¹H NMR (400 MHz, CD₃OD) δ 7.65 (d, J=1.6 Hz, 1H), 7.29 (d, J=3.2 Hz, 1H), 7.10-6.90 (m, 7H), 6.83-6.75 (m, 1H), 6.70-6.60 (m, 2H), 6.58 (d, J=3.2 Hz, 1H), 6.46-6.44 (m, 1H), 5.40-5.37 (m, 1H), 4.15-4.05 (m, 2H), 3.92-3.74 (m, 2H), 3.19-3.16 (m, 1H), 2.86-2.81 (m, 3H), 2.66-2.47 (m, 3H), 2.23-2.12 (m, 1H), 0.95 (s, 3H), 0.60 (s, 3H) ppm.

Compound 90A: MS (ESI): 652 m/z [M+H]⁺, retention time: 1.11 minutes, purity: >99% (214 nm) (LC-MS method 22). Chiral-HPLC: ee %: 100%. ¹H NMR (400 MHz, CD₃OD) δ 7.53 (d, J=2.0 Hz, 1H), 7.43-7.41 (m, 1H), 7.29 (s, 1H), 7.20 (d, J=10.8 Hz, 1H), 7.16-7.12 (m, 2H), 7.10-6.95 (m, 6H), 6.58-6.56 (m, 1H), 6.17 (d, J=3.2 Hz, 1H), 5.30 (dd, J=12.0, 2.0 Hz, 1H), 4.14-4.00 (m, 2H), 3.79-3.65 (m, 2H), 3.35-3.22 (m, 1H), 3.20-3.18 (m, 1H), 2.82 (t, J=8.0 Hz, 2H), 2.51-2.46 (m, 3H), 1.96-1.93 (m, 1H), 0.99 (s, 3H), 0.76 (s, 3H) ppm.

Compound 90B: MS (ESI): 652 m/z [M+H]⁺, retention time: 1.12 minutes, purity: 98% (214 nm) (LC-MS method 22). Chiral-HPLC: ee %: 97%; H NMR (400 MHz, CD₃OD) δ 7.53 (d, J=2.4 Hz, 1H), 7.43-7.41 (m, 1H), 7.29 (s, 1H), 7.21 (d, J=10.8 Hz, 1H), 7.16-7.12 (m, 2H), 7.10-6.95 (m, 6H), 6.58-6.56 (m, 1H), 6.17 (d, J=3.2 Hz, 1H), 5.32-5.27 (m, 1H), 4.14-4.00 (m, 2H), 3.80-3.66 (m, 2H), 3.25-3.20 (m, 1H), 3.11-3.07 (m, 1H), 2.82 (t, J=8.0 Hz, 2H), 2.49-2.46 (m, 3H), 1.96-1.93 (m, 1H), 1.00 (s, 3H), 0.77 (s, 3H) ppm.

Example 92. Compound 92A and Compound 92B. Enantiomers 1 and 2 of 3-[3-(24,30-difluoro-3,6,11,11-tetramethyl-26-oxa-3,4,14,15,21,33-hexazahexacyclo-[25.3.1.12,5.112,15.017,25.018,22]tritriaconta-1(31),2(33),4,12(32),13,17,19,22,24,27,29-undecaen-6-yl)phenyl]propanoic acid

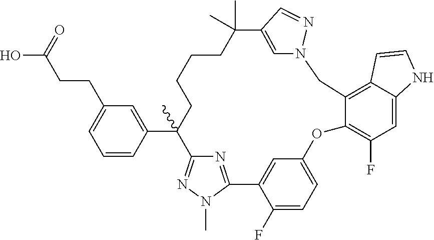 [SMILES: Cn1nc2nc1-c1cc(ccc1F)Oc1c(F)cc3[nH]ccc3c1Cn1cc(cn1)C(C)(C)CCCCC2(C)c1cccc(CCC(=O)O)c1]

5-(3-(3-(2-(3-Bromophenyl)pent-4-en-2-yl)-1-methyl-1H-1,2,4-triazol-5-yl)-4-fluorophenoxy)-6-fluoro-4-((4-(2-methylpent-4-en-2-yl)-1H-pyrazol-1-yl)methyl)-1-(phenylsulfonyl)-1H-indole

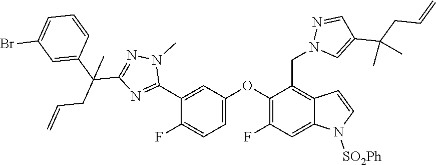

Step A: To a stirred solution of methyl 2-fluoro-5-((6-fluoro-4-((4-(2-methylpent-4-en-2-yl)-1H-pyrazol-1-yl)methyl)-1-(phenylsulfonyl)-1H-indol-5-yl)oxy)benzimidothioate (Intermediate 82, 1.6 g, 2.58 mmol) and 2-(3-bromophenyl)-N′,2-dimethylpent-4-enehydrazide (Intermediate 83, 766 mg, 2.58 mmol) in pyridine (20 mL) was added magnesium sulfate (1.6 g). The mixture was heated to 80° C. and stirred overnight, then cooled to room temperature and quenched with water (100 mL). The mixture was extracted with ethyl acetate (3×50 mL). The combined organic phases were washed with brine (20 mL), dried over sodium sulfate, and concentrated. The residue was purified by automated flash chromatography (80 g silica gel column, eluting with 0-100% ethyl acetate in petroleum ether) to afford the title compound (1.2 g, 55%) as solid. MS (ESI): 851, 853 m/z [M+H]⁺, retention time: 1.89 minutes, purity: 71% (214 nm) (LC-MS Method 24).

(8Z)-21-(Benzenesulfonyl)-6-(3-bromophenyl)-24,30-difluoro-3,6,11,11-tetramethyl-26-oxa-3,4,14,15,21,33-hexazahexacyclo[25.3.1.12,5.112,15.017,25.018,22]tritriaconta-1(31),2(33),4,8,12(32),13,17,19,22,24,27,29-dodecaene

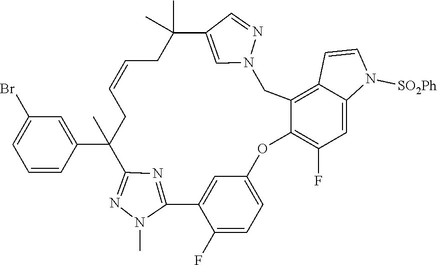

Step B: To a stirred solution of Step A product (1.24 g, 1.46 mmol) in toluene was added Grubb's catalyst 2nd generation (247 mg, 0.3 mmol). The mixture was heated to 100° C. and stirred for 16 hours and concentrated. The residue was purified by automated flash chromatography (80 g silica gel column, eluting with 0-70% ethyl acetate in petroleum ether) to afford the title compound (740 mg, 62%) as solid. MS (ESI): 823, 825 m/z [M+H]⁺, retention time: 1.82 minutes, purity: 80% (214 nm) (LC-MS Method 24).

(8Z)-6-(3-Bromophenyl)-24,30-difluoro-3,6,11,11-tetramethyl-26-oxa-3,4,14,15,21,33-hexazahexacyclo[25.3.1.12,5.112,15.017,25.018,22]tritriaconta-1(31),2(33),4,8,12(32),13,17,19,22,24,27,29-dodecaene

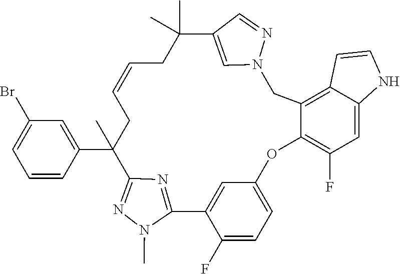 [SMILES: Cn1nc2nc1-c1cc(ccc1F)Oc1c(F)cc3[nH]ccc3c1Cn1cc(cn1)C(C)(C)C/C=C\CC2(C)c1cccc(Br)c1]

Step C: To a stirred solution of Step B product (740 mg, 0.9 mmol) in methanol (10 mL) was added sodium hydroxide (108 mg, 2.7 mmol). The mixture was stirred at room temperature for 16 hours, diluted with water, adjusted pH to ˜6 with 1 N hydrochloric acid. The mixture was extracted with ethyl acetate (3×50 mL). The combined organic phases were washed with brine (20 mL), dried over sodium sulfate, and concentrated. The crude material was purified by flash chromatography (40 g silica gel column, eluting with 0-70% ethyl acetate in petroleum ether) to afford the title compound (500 mg, 82%) as solid. MS (ESI): 683, 685 m/z [M+H]⁺, retention time: 1.68 minutes, purity: 81% (214 nm) (LC-MS Method 24).

Ethyl (E)-3-[3-[(8Z)-24,30-difluoro-3,6,11,11-tetramethyl-26-oxa-3,4,14,15,21,33-hexazahexacyclo[25.3.1.12,5.112,15.017,25.018,22]tritriaconta-1(31),2(33),4,8,12(32),13,17,19,22,24,27,29-dodecaen-6-yl]phenyl]prop-2-enoate

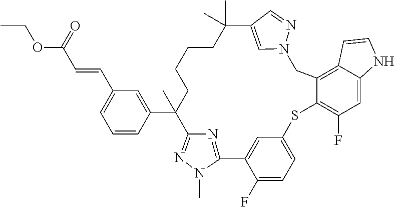 [SMILES: CCOC(=O)/C=C/c1cccc(C2(C)CCCCC(C)(C)c3cnn(c3)Cc3c(c(F)cc4[nH]ccc34)Sc3ccc(F)c(c3)-c3nc2nn3C)c1]

Step D: To a stirred and degassed solution of Step C product (0.5 g, 0.73 mmol) in N-methyl-pyrrolidone (10 mL) was added ethyl acrylate (219 mg, 2.19 mmol), palladium (II) acetate (24.6 mg, 0.1 mmol), tri-o-tolyl-phosphine (77 mg, 0.22 mmol) and triethylamine (0.37 g, 3.65 mmol). The mixture was stirred at 130° C. for 2 hours in a microwave reactor. The mixture was diluted with ethyl acetate (100 mL). The solution was washed with water, brine, dried over sodium sulfate, and concentrated. The crude material was purified by automated flash chromatography (40 g silica gel column, eluting with 0-10% dichloromethane in methanol) to afford the title compound (400 mg, 78%) as a light-yellow solid. MS (ESI): 703 m/z [M+H]⁺, retention time: 1.42 minutes, purity: 90% (214 nm) (LC-MS Method 2).

Ethyl 3-[3-(24,30-difluoro-3,6,11,11-tetramethyl-26-oxa-3,4,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]propanoate

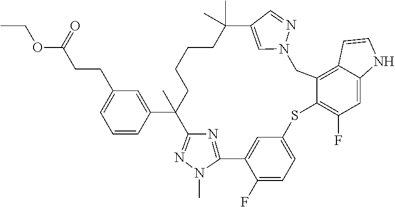 [SMILES: CCOC(=O)CCc1cccc(C2(C)CCCCC(C)(C)c3cnn(c3)Cc3c(c(F)cc4[nH]ccc34)Sc3ccc(F)c(c3)-c3nc2nn3C)c1]

Step E: To a stirred solution of Step D product (400 mg, 0.57 mmol) in ethyl acetate (10 mL) was added palladium on carbon (10%, 50% wet, 400 mg). The mixture was stirred at 50° C. under hydrogen atmosphere overnight, then filtered through a pad of Celite. The filtrate was concentrated to give the crude title compound (330 mg, 82%) as a light-yellow solid. MS (ESI): 707 m/z [M+H]⁺, retention time: 1.55 minutes, purity: 86% (214 nm) (LC-MS Method 2).

Enantiomers 1 and 2 of ethyl 3-[3-(24,30-difluoro-3,6,11,11-tetramethyl-26-oxa-3,4,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]propanoate

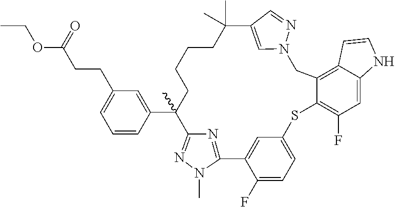 [SMILES: CCOC(=O)CCc1cccc(C2(C)CCCCC(C)(C)c3cnn(c3)Cc3c(c(F)cc4[nH]ccc34)Sc3ccc(F)c(c3)-c3nc2nn3C)c1]

Step F: The racemic Step E product (330 mg) was subject to chiral SFC separation using SFC-80 (Thar, Waters) under the following condition: Column: OD 20*250 mm, 10 μm; Column temperature: 35° C.; Mobile phase: carbon dioxide/methanol (0.2% methanol ammonia)=70/30; Flow rate: 60 g/minute; Back pressure: 100 bar; Detection wavelength: 214 nm; Cycle time:5.3 minutes; Sample solution: 0.33 g dissolved in 25 mL methanol; Injection volume:1.5 mL. The first eluent is designated as Enantiomer 1 (130 mg, 39%) and the second eluent is designated as Enantiomer 2 (110 mg, 33%).

Compound 92A. Enantiomer 1 of 3-[3-(24,30-difluoro-3,6,11,11-tetramethyl-26-oxa-3,4,14,15,21,33-hexazahexacyclo-[25.3.1.12,5.112,15.017,25.018,22]tritriaconta-1(31),2(33),4,12(32),13,17,19,22,24,27,29-undecaen-6-yl)phenyl]propanoic acid

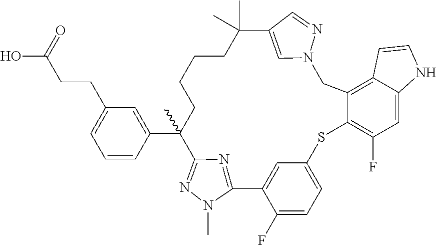 [SMILES: Cn1nc2nc1-c1cc(ccc1F)Sc1c(F)cc3[nH]ccc3c1Cn1cc(cn1)C(C)(C)CCCCC2(C)c1cccc(CCC(=O)O)c1]

Step G: To a solution of Enantiomer 1 of Step F product (130 mg, 0.18 mmol) was added lithium hydroxide solution (0.25 M lithium hydroxide in water/tetrahydrofuran/methanol=1/1/3, 10 mL). The mixture was stirred at room temperature for 3 hours, diluted with water (20 mL), acidified to pH ˜5 with 1 N hydrochloric acid. The mixture was extracted with ethyl acetate (3×20 mL). The combined organic phases were washed with water, brine, dried over sodium sulfate and concentrated. The residue was purified by prep-TLC (dichloromethane\methanol=90†10) to give the title compound (60 mg, 48%). MS (ESI): 679 m/z [M+H]⁺, retention time: 1.77 minutes, purity: 98% (214 nm) (LC-MS Method 8). ¹H NMR (400 MHz, CD₃OD) δ 7.41-7.28 (m, 4H), 7.27 (s, 1H), 7.20 (s, 1H), 7.18-7.12 (m, 1H), 7.08 (s, 1H), 7.04-6.99 (m, 2H), 6.63 (dd, J=5.6, 3.2 Hz, 1H), 6.49-6.47 (m, 1H), 5.59 (d, J=14.4 Hz, 1H), 5.54 (d, J=14.4 Hz, 1H), 3.80 (d, J=2.8 Hz, 3H), 2.85 (t, J=7.6 Hz, 2H), 2.53 (t, J=7.6 Hz, 2H), 2.26-2.18 (m, 1H), 2.08-1.98 (m, 1H), 1.63 (s, 3H), 1.49-1.41 (m, 2H), 1.38-1.26 (m, 2H), 1.10 (s, 3H), 1.06 (s, 3H), 0.95-0.90 (m, 1H), 0.85-0.77 (m, 1H) ppm.

Compound 92B. Enantiomer 2 of 3-[3-(24,30-difluoro-3,6,11,11-tetramethyl-26-oxa-3,4,14,15,21,33-hexazahexacyclo-[25.3.1.12,5.112,15.017,25.018,22]tritriaconta-1(31),2(33),4,12(32),13,17,19,22,24,27,29-undecaen-6-yl)phenyl]propanoic acid

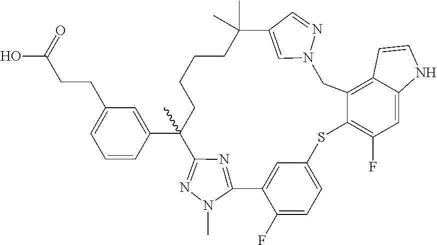 [SMILES: Cn1nc2nc1-c1cc(ccc1F)Sc1c(F)cc3[nH]ccc3c1Cn1cc(cn1)C(C)(C)CCCCC2(C)c1cccc(CCC(=O)O)c1]

Step H: Enantiomer 2 of Step F product (110 mg, 0.16 mmol) was subjected the identical conditions as described in Step G to afford the title compound (60 mg, 54%). MS (ESI): 679 m/z [M+H]⁺, retention time: 1.77 minutes, purity: 99% (214 nm) (LC-MS Method 8). ¹H NMR (400 MHz, CD₃OD) δ 7.41-7.25 (m, 4H), 7.27 (s, 1H), 7.20 (s, 1H), 7.18-7.12 (m, 1H), 7.08 (s, 1H), 7.04-6.99 (m, 2H), 6.63 (dd, J=5.6, 3.2 Hz, 1H), 6.49-6.47 (m, 1H), 5.59 (d, J=14.4 Hz, 1H), 5.54 (d, J=14.4 Hz, 1H), 3.80 (d, J=2.8 Hz, 3H), 2.85 (t, J=7.6 Hz, 2H), 2.53 (t, J=7.6 Hz, 2H), 2.26-2.18 (m, 1H), 2.08-1.98 (m, 1H), 1.63 (s, 3H), 1.49-1.41 (m, 2H), 1.38-1.26 (m, 2H), 1.10 (s, 3H), 1.06 (s, 3H), 0.95-0.90 (m, 1H), 0.85-0.77 (m, 1H) ppm.

Example 93. Compound 93A and Compound 93B. Enantiomers 1 and 2 of 3-[3-(24,30-difluoro-3,6,32-trimethyl-10,26-dioxa-3,4,13,14,15,21,33-heptazahexacyclo-[25.3.1.12,5.112,15.017,25.018,22]tritriaconta-1(31),2(33),4,12(32),13,17,19,22,24,27,29-undecaen-6-yl)phenyl]propanoic acid

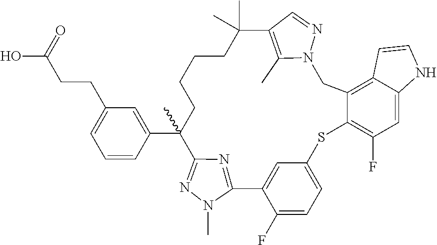 [SMILES: Cc1c2cnn1Cc1c(c(F)cc3[nH]ccc13)Sc1ccc(F)c(c1)-c1nc(nn1C)C(C)(c1cccc(CCC(=O)O)c1)CCCCC2(C)C]

S-(3-(3-(2-(3-Bromophenyl)-5-(but-2-yn-1-yloxy)pentan-2-yl)-1-methyl-1H-1,2,4-triazol-5-yl)-4-fluorophenoxy)-6-fluoro-4-vinyl-1H-indole

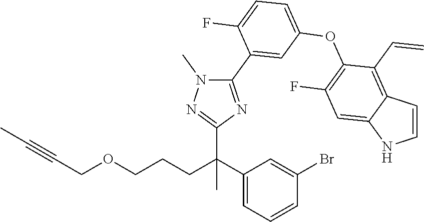 [SMILES: C=Cc1c(Oc2ccc(F)c(-c3nc(C(C)(CCCOCC#CC)c4cccc(Br)c4)nn3C)c2)c(F)cc2[nH]ccc12]

Step A: To a stirred solution of 2-(3-bromophenyl)-5-(but-2-yn-1-yloxy)-N′,2-dimethylpentanehydrazide (Intermediate 83-1, 2 g, 5.45 mmol) in pyridine (30 mL) was added methyl 2-fluoro-5-((6-fluoro-4-vinyl-1H-indol-5-yl)oxy)benzimidothioate hydroiodide (Intermediate 82-1, 2.8 g, 5.99 mmol). The mixture was stirred at 80° C. overnight, cooled to room temperature and quenched with water (100 mL). The solution was extracted with ethyl acetate (2×80 mL). The combined organic phases were washed with hydrochloric acid (1M), brine, dried over sodium sulfate, and concentrated. The residue was purified by automated silica gel column chromatography (80 g silica gel column, eluting with 0-50% ethyl acetate in petroleum ether) to give the title compound (1.8 g, 51%) as a solid. MS (ESI): 645, 647 m/z [M+H]⁺, retention time: 2.19 minutes, purity: 97% (214 nm) (LC-MS Method 5).

1-(5-(3-(3-(2-(3-Bromophenyl)-5-(but-2-yn-1-yloxy)pentan-2-yl)-1-methyl-1H-1,2,4-triazol-5-yl)-4-fluorophenoxy)-6-fluoro-1H-indol-4-yl)ethane-1,2-diol

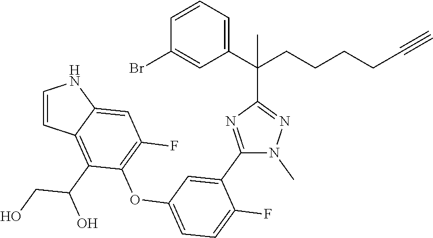 [SMILES: C#CCCCCC(C)(c1cccc(Br)c1)c1nc(-c2cc(Oc3c(F)cc4[nH]ccc4c3C(O)CO)ccc2F)n(C)n1]

Step B: To a stirred solution of Step A product (1.8 g, 2.79 mmol) in acetone (45 mL) was added 4-methylmorpholine n-oxide (0.93 mL, 4.46 mmol) and osmium tetroxide (28.4 mg, 0.112 mmol). The mixture was stirred at room temperature overnight, quenched with water (200 mL), and extracted with ethyl acetate (2×150 mL). The combined organic phases were washed with brine, dried over sodium sulfate, and concentrated to the crude title compound (1.4 g, 74%) as a solid. MS (ESI): 679, 681 m/z [M+H]⁺, retention time: 1.89 minutes, purity: 80% (214 nm) (LC-MS Method 5).

S-(3-(3-(2-(3-Bromophenyl)-5-(but-2-yn-1-yloxy)pentan-2-yl)-1-methyl-1H-1,2,4-triazol-5-yl)-4-fluorophenoxy)-6-fluoro-1H-indole-4-carbaldehyde

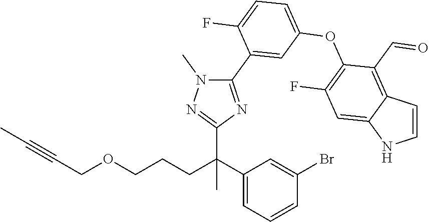 [SMILES: CC#CCOCCCC(C)(c1cccc(Br)c1)c1nc(-c2cc(Oc3c(F)cc4[nH]ccc4c3C=O)ccc2F)n(C)n1]

Step C: To a stirred solution of Step B product (1.4 g, 2.06 mmol) in acetone (15 mL) and water (5 mL) was added sodium periodate (1.32 g, 6.18 mmol). The mixture was stirred at room temperature overnight. The solution was quenched with water (80 mL) and extracted with ethyl acetate (2×60 mL). The combined organic phases were washed with brine, dried over sodium sulfate, and concentrated. The residue was purified by silica gel column chromatography (40 g silica gel column, eluting with 0-60% ethyl acetate in petroleum ether) to give the title compound (530 mg, 40%) as a solid. MS (ESI): 647, 649 m/z [M+H]⁺, retention time: 2.08 minutes, purity: 93% (214 nm) (LC-MS Method 5).

(5-(3-(3-(2-(3-Bromophenyl)-5-(but-2-yn-1-yloxy)pentan-2-yl)-1-methyl-1H-1,2,4-triazol-5-yl)-4-fluorophenoxy)-6-fluoro-1H-indol-4-yl)methanol

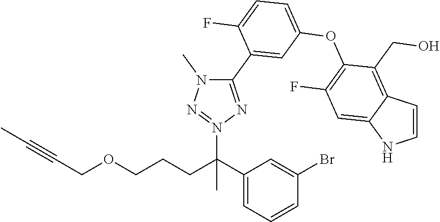

Step D: To a stirred solution of Step C product (530 mg, 0.819 mmol) in tetrahydrofuran (10 mL) was added sodium borohydride (47 mg, 1.23 mmol). The mixture was stirred at room temperature for four hours, quenched with water (40 mL), and extracted with ethyl acetate (2×30 mL). The combined organic phases were washed with brine, dried over sodium sulfate, and concentrated to give the crude title compound (530 mg, 99%) as a solid, which was used for next step without further purification. MS (ESI): 649, 651 m/z [M+H]⁺, retention time: 1.97 minutes, purity: 90% (214 nm) (LC-MS Method 5).

4-(Azidomethyl)-5-(3-(3-(2-(3-bromophenyl)-5-(but-2-yn-1-yloxy)pentan-2-yl)-1-methyl-1H-1,2,4-triazol-5-yl)-4-fluorophenoxy)-6-fluoro-1H-indole

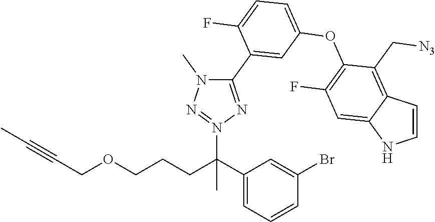

Step E: To a stirred solution of Step D product (530 mg, 0.816 mmol) in tetrahydrofuran (10 mL) was added 1,8-diazabicyclo[5.4.0]undec-7-ene (248 mg, 1.63 mmol) and diphenylphosphoryl azide (674 mg, 2.45 mmol), then the mixture was stirred at 70° C. overnight and concentrated. The residue was purified by automated silica gel column chromatography (40 g silica gel column, eluting with 0-60% ethyl acetate in petroleum ether) to give the title compound (350 mg, 64%) as a solid. MS (ESI): 674, 676 m/z [M+H]⁺, retention time: 2.15 minutes, purity: 92% (214 nm) (LC-MS Method 5).

6-(3-Bromophenyl)-24,30-difluoro-3,6,32-trimethyl-10,26-dioxa-3,4,13,14,15,21,33-heptazahexacyclo[25.3.1.12,5.112,15.017,25.018,22]tritriaconta-1(31),2(33),4,12(32),13,17,19,22,24,27,29-undecaene

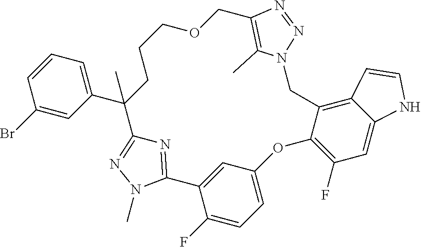 [SMILES: Cc1c2nnn1Cc1c(c(F)cc3[nH]ccc13)Oc1ccc(F)c(c1)-c1nc(nn1C)C(C)(c1cccc(Br)c1)CCCOC2]

Step F: To a stirred solution of Step E product (600 mg, 0.889 mmol) in toluene (180 mL) was added chloro(1,5-cyclooctadiene)(pentamethylcyclopentadienyl)ruthenium (67.6 mg, 0.178 mmol). The mixture was stirred at 60° C. overnight. The solvent was removed. The residue was purified by automated silica gel column chromatography (40 g silica gel column, eluting with 0-80% ethyl acetate in petroleum ether) to give the title compound (410 mg, 68%) as a solid. MS (ESI): 674,676 m/z [M+H]⁺, retention time: 1.94 minutes, purity: 95% (214 nm) (LC-MS Method 5).

Compounds 93A, 93B. Enantiomers 1 and 2 of 3-[3-(24,30-difluoro-3,6,32-trimethyl-10,26-dioxa-3,4,13,14,15,21,33-heptazahexacyclo-[25.3.1.12,5.112,15.017,25.018,22]tritriaconta-1(31),2(33),4,12(32),13,17,19,22,24,27,29-undecaen-6-yl)phenyl]propanoic acid

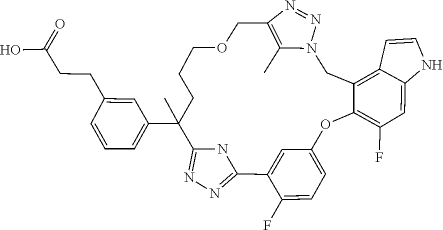 [SMILES: Cc1c2nnn1Cc1c(c(F)cc3[nH]ccc13)Oc1ccc(F)c(c1)-c1nnc([nH]1)C(C)(c1cccc(CCC(=O)O)c1)CCCOC2]

Step G: Exchanging 23,29-difluoro-6-(3-iodophenyl)-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaene (Step B product of Example 2) with 6-(3-bromophenyl)-24,30-difluoro-3,6,32-trimethyl-10,26-dioxa-3,4,13,14,15,21,33-heptazahexacyclo[25.3.1.12,5.112,15.017,25.018,22]tritriaconta-1(31),2(33),4,12(32),13,17,19,22,24,27,29-undecaene (Step F product of this Example, 410 mg, 0.61 mmol), the reaction procedure sequence (Steps C to E) described for Example 2 was used to prepare the title compounds. The racemic ethyl ester (380 mg, 0.546 mmol), obtained after hydrogenation of corresponding Step D of Example 2, was subject to chiral SFC separation using SFC-80 (Thar, Waters) under the following condition: Column: AS 20*250 mm, 10 μm; Column temperature: 35° C.; Mobile phase: carbon dioxide/methanol (0.2% methanol ammonia)=65/35; Flow rate: 80 g/minute; Back pressure: 100 bar; Detection wavelength: 214 nm; Cycle time: 8.0 minutes; Sample solution: 380 mg dissolved in 28 mL methanol. The first eluent, Enantiomer 1 (130 mg, 34%), was further hydrolyzed to Compound 93A (110 mg, 88%); The second, Enantiomer 2 (130 mg, 34%), was further hydrolyzed to Compound 93B (110 mg, 88%), according to the conditions described in Step E of Example 2.

Compound 93A: MS (ESI): 668 m/z [M+H]⁺, retention time: 1.70 minutes, purity: 98% (214 nm) (LC-MS Method 5). ¹H NMR (400 MHz, CD₃OD) δ 7.47 (d, J=3.2 Hz, 1H), 7.34 (d, J=10.4 Hz, 1H), 7.24-7.18 (m, 2H), 7.14-7.07 (m, 3H), 7.00-6.96 (m, 1H), 6.89 (d, J=2.8 Hz, 1H), 6.31-6.28 (m, 1H), 6.04 (d, J=15.2 Hz, 1H), 5.77 (d, J=15.2 Hz, 1H), 4.52 (d, J=11.2 Hz, 1H), 4.16 (d, J=11.2 Hz, 1H), 3.79 (d, J=3.0 Hz, 3H), 3.52-3.46 (m, 2H), 2.80-2.74 (m, 1H), 2.64 (t, J=7.2 Hz, 2H), 2.40 (t, J=7.2 Hz, 2H), 2.31 (s, 3H), 1.81-1.76 (m, 1H), 1.71 (s, 3H), 1.55-1.44 (m, 2H) ppm.

Compound 93B: MS (ESI): 668 m/z [M+H]⁺, retention time: 1.70 minutes, purity: 99% (214 nm) (LC-MS Method 5). ¹H NMR (400 MHz, CD₃OD) δ 7.47 (d, J=3.2 Hz, 1H), 7.34 (d, J=10.4 Hz, 1H), 7.24-7.18 (m, 2H), 7.14-7.07 (m, 3H), 7.00-6.96 (m, 1H), 6.89 (d, J=2.8 Hz, 1H), 6.31-6.28 (m, 1H), 6.04 (d, J=15.2 Hz, 1H), 5.77 (d, J=15.2 Hz, 1H), 4.52 (d, J=11.2 Hz, 1H), 4.16 (d, J=11.2 Hz, 1H), 3.79 (d, J=3.0 Hz, 3H), 3.52-3.46 (m, 2H), 2.80-2.74 (m, 1H), 2.64 (t, J=7.2 Hz, 2H), 2.40 (t, J=7.2 Hz, 2H), 2.31 (s, 3H), 1.81-1.76 (m, 1H), 1.71 (s, 3H), 1.55-1.44 (m, 2H) ppm.

Example 94. Compound 94A and Compound 94B. 3-[3-(24,30-Difluoro-3,6,9,9-tetramethyl-26-oxa-3,4,12,21,32,33-hexazahexacyclo[25.3.1.12,5.112,15.017,25.018,22]-tritriaconta-1(31),2(33),4,13,15(32),17,19,22,24,27,29-undecaen-6-yl)phenyl]propanoic acid

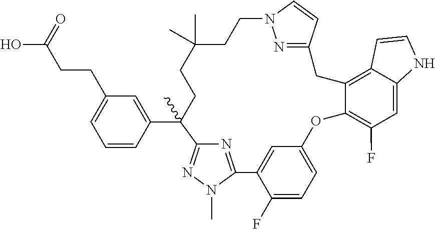 [SMILES: Cn1nc2nc1-c1cc(ccc1F)Oc1c(F)cc3[nH]ccc3c1Cc1ccn(n1)CCC(C)(C)CCC2(C)c1cccc(CCC(=O)O)c1]

6-(3-Bromophenyl)-6-(5-(2-fluoro-5-((6-fluoro-1-(phenylsulfonyl)-4-((1-(tetrahydro-2H-pyran-2-yl)-1H-pyrazol-3-yl)methyl)-1H-indol-5-yl)oxy)phenyl)-1-methyl-1H-1,2,4-triazol-3-yl)-3,3-dimethylheptan-1-ol

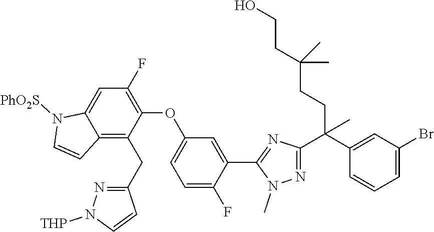

Step A: To a stirred solution of methyl 2-fluoro-5-((6-fluoro-1-(phenylsulfonyl)-4-((1-(tetrahydro-2H-pyran-2-yl)-1H-pyrazol-3-yl)methyl)-1H-indol-5-yl)oxy)benzimidothioate (Intermediate 82-2, 6.4 g, 10 mmol) in pyridine (40 mL) was added 2-(3-bromophenyl)-7-hydroxy-N′,2,5,5-tetramethylheptanehydrazide (Intermediate 83-2, 3.7 g, 10 mmol). The mixture was heated to 80° C. and stirred for 16 hours, cooled to room temperature, and quenched with water (100 mL). The solution was extracted with ethyl acetate (3×100 mL). The combined organic phases were washed with water, brine, dried over sodium sulfate, and concentrated. The residue was purified by flash chromatography to afford the title compound (5.0 g, 54%). MS (ESI): 927,929 m/z [M+H]⁺, retention time: 2.52 minutes, purity: 90% (214 nm) (LC-MS Method 25).

6-(5-(5-((4-((1H-Pyrazol-3-yl)methyl)-6-fluoro-1-(phenylsulfonyl)-1H-indol-5-yl)oxy)-2-fluorophenyl)-1-methyl-1H-1,2,4-triazol-3-yl)-6-3-bromophenyl)-3,3-dimethylheptan-1-ol

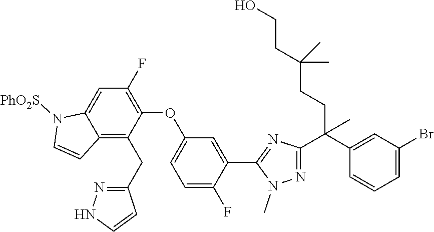

Step B: To a stirred solution of Step A product (4.9 g, 5.28 mmol) in methanol (40 mL) was added p-toluene-sulfonic acid (2.65 g, 10.6 mmol). The resulting mixture was heated to reflux and stirred for 24 hours and concentrated. The residue was partitioned between water (100 mL) and ethyl acetate (100 mL). The separated organic layer, combined with two additional ethyl acetate extracts (2×50 mL), were washed with saturated sodium bicarbonate, dried over sodium sulfate, and concentrated. The residue was purified by flash chromatography to afford the title compound (3.5 g, 78%). MS (ESI): 843, 845 m/z [M+H]⁺, retention time: 2.32 minutes, purity: 90% (254 nm) (LC-MS Method 25).

21-(Benzenesulfonyl)-6-(3-bromophenyl)-24,30-difluoro-3,6,9,9-tetramethyl-26-oxa-3,4,12,21,32,33-hexazahexacyclo[25.3.1.12,5.112,15.017,25.018,22]tritriaconta-1(31),2(33),4,13,15(32),17,19,22,24,27,29-undecaene

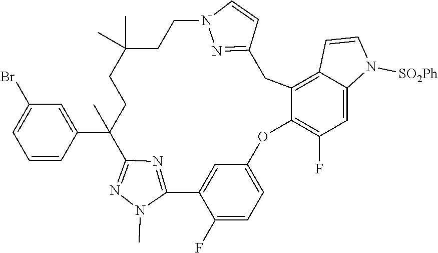

Step C: To a stirred solution of Step B product (250 mg, 0.29 mmol) in 1,4-dioxane (10 mL) was added (tributylphosphoranylidene)acetonitrile (0.89 mmol, 273 mg) in a microwave vial. The mixture was heated to 150° C. for 50 minutes in a microwave reactor. The solvent was removed. The residue was purified by flash chromatography to afford the title compound (100 mg, 40%). MS (ESI): 825, 827 m/z [M+H]⁺, retention time: 1.81 minutes, purity: 90% (254 nm) (LC-MS Method 25).

6-(3-Bromophenyl)-24,30-difluoro-3,6,9,9-tetramethyl-26-oxa-3,4,12,21,32,33-hexazahexacyclo-[25.3.1.12,5.112,15.017,25.018,22]tritriaconta-1(31),2(33),4,13,15(32),17,19,22,24,27,29-undecaene

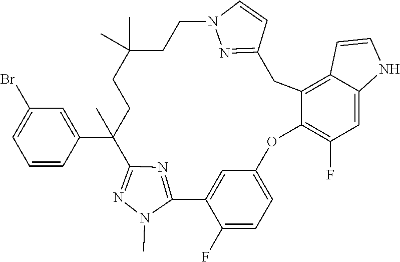 [SMILES: Cn1nc2nc1-c1cc(ccc1F)Oc1c(F)cc3[nH]ccc3c1Cc1ccn(n1)CCC(C)(C)CCC2(C)c1cccc(Br)c1]

Step D: To a stirred solution of Step C product (0.57 mmol, 560 mg) was added 0.2 M lithium hydroxide (in methanol:water:tetrahydrofuran=1:1:3, 13.6 mL). The mixture was stirred at room temperature for 6 hours, then acidified with 8 mL of 1 N hydrochloric acid and extracted with ethyl acetate (2×30 mL). The combined organic phases were washed with brine, dried over sodium sulfate, and concentrated. The residue was purified by flash chromatography to afford the title compound (430 mg, 95%). MS (ESI): 685, 687 m/z [M+H]⁺, retention time: 2.51 minutes, purity: >99% (254 nm) (LC-MS Method 25).

Compound 94A and 94B. Enantiomers 1 and 2 of 3-[3-(24,30-Difluoro-3,6,9,9-tetramethyl-26-oxa-3,4,12,21,32,33-hexazahexacyclo-[25.3.1.12,5.112,15.017,25.018,22]-tritriaconta-1(31),2(33),4,13,15(32),17,19,22,24,27,29-undecaen-6-yl)phenyl]propanoic acid

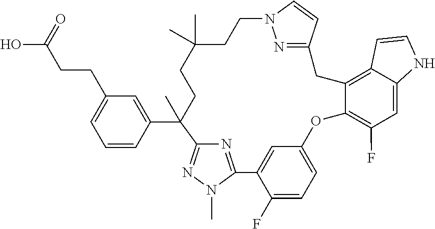 [SMILES: Cn1nc2nc1-c1cc(ccc1F)Oc1c(F)cc3[nH]ccc3c1Cc1ccn(n1)CCC(C)(C)CCC2(C)c1cccc(CCC(=O)O)c1]

Exchanging 23,29-difluoro-6-(3-iodophenyl)-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaene (Step B product of Example 2) with 6-(3-bromophenyl)-24,30-difluoro-3,6,9,9-tetramethyl-26-oxa-3,4,12,21,32,33-hexazahexacyclo[25.3.1.12,5.112,15.017,25.018,22]tritriaconta-1(31),2(33),4,13,15(32),17,19,22,24,27,29-undecaene (Step D product of this example, 400 mg, 0.58 mmol), the reaction procedure sequence (Steps C, D and E) described for Example 2 was used to prepare the title compounds. The racemic acid (270 mg), obtained from corresponding Step E of Example 2, was subject to chiral SFC separation using SFC-80 (Thar, Waters) under the following condition: Column: SSWHELK 20*250 mm, 10 μm; Column temperature: 35° C.; Mobile phase: carbon dioxide/methanol (0.2% methanol ammonia)=30/70; Flow rate: 80 g/minute; Back pressure: 100 bar; Detection wavelength: 214 nm; Cycle time: 10.4 minutes; Sample solution: 0.27 g dissolved in 40 mL methanol; Injection volume: 4.5 mL. The first eluent, Enantiomer 1, is designated as Compound 94A (64.5 mg, 24%); The second eluent, Enantiomer 2, is designated as Compound 94B (30.1 mg, 11%).

Compound 94A: MS (ESI): 679 m/z [M+H]⁺, retention time: 2.24 minutes (LC-MS Method 5). ¹H NMR (400 MHz, DMSO-d₆) δ 11.24 (s, 1H), 7.40-6.97 (m, 10H), 6.52 (s, 1H), 5.91 (d, J=2.0 Hz, 1H), 4.10 (s, 2H), 3.82-3.60 (m, 5H), 2.74 (t, J=7.6 Hz, 2H), 2.46 (t, J=7.6 Hz, 2H), 2.22 (t, J=11.9 Hz, 1H), 1.82-1.53 (m, 6H), 1.23-1.19 (m, 2H), 0.86 (s, 3H), 0.83 (s, 3H) ppm.

Compound 94B: MS (ESI): 679 m/z [M+H]⁺, retention time: 2.24 minutes (LC-MS Method 5). ¹H NMR (400 MHz, DMSO-d₆) δ 11.24 (s, 1H), 7.40-6.97 (m, 10H), 6.52 (s, 1H), 5.91 (d, J=2.0 Hz, 1H), 4.10 (s, 2H), 3.82-3.61 (m, 5H), 2.74 (t, J=7.6 Hz, 2H), 2.46 (t, J=7.6 Hz, 2H), 2.22 (t, J=11.9 Hz, 1H), 1.82-1.53 (m, 6H), 1.23-1.18 (m, 2H), 0.86 (s, 3H), 0.83 (s, 3H) ppm.

Example 95. Compound 95. 3-[3-(11,11,24,30-tetrafluoro-10-hydroxy-3,6,9,9-tetramethyl-26-oxa-3,4,13,14,15,21,33-heptazahexacyclo[25.3.1.12,5.112,15.017,25.018,22]tritriaconta-1(31),2(33),4,12(32),13,17,19,22,24,27,29-undecaen-6-yl)phenyl]propanoic acid

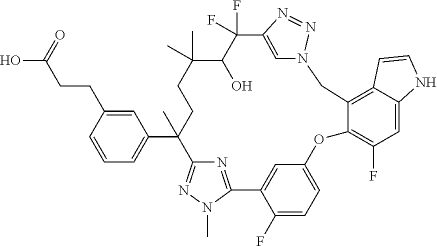 [SMILES: Cn1nc2nc1-c1cc(ccc1F)Oc1c(F)cc3[nH]ccc3c1Cn1cc(nn1)C(F)(F)C(O)C(C)(C)CCC2(C)c1cccc(CCC(=O)O)c1]

Exchanging 2-(3-bromophenyl)-5-(but-2-yn-1-yloxy)-N′,2-dimethylpentanehydrazide (Intermediate 83-1) with 2-(3-bromophenyl)-7,7-difluoro-6-hydroxy-N′,2,5,5-tetramethylnona-8-ynehydrazide (Intermediate 97H-2, 2 g, 4.64 mmol), the reaction procedure sequence (Steps A to G) described for Example 93A, 93B was used to prepare the title compound as a cis/trans diastereomeric mixture (15 mg). MS (ESI): 732 m/z [M+H]⁺, retention time: 2.04+2.11 minutes (cis/trans isomers); purity: 49%+47% (214, cis/trans isomers) (LC-MS Method 5).

Example 96. Compound 96A and 96B. Enantiomers 1 and 2 of 3-[3-(24,30-difluoro-3,6,11,11-tetramethyl-26-oxa-3,4,13,14,15,21,33-heptazahexacyclo-[25.3.1.12,5.112,15.017,25.018,22]tritriaconta-1(31),2(33),4,12(32),13,17,19,22,24,27,29-undecaen-6-yl)phenyl]-2-methyl-propanoic acid

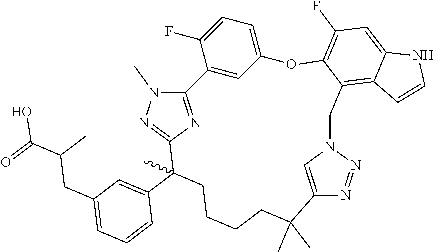 [SMILES: CC(Cc1cccc(C2(C)CCCCC(C)(C)c3cn(nn3)Cc3c(c(F)cc4[nH]ccc34)Oc3ccc(F)c(c3)-c3nc2nn3C)c1)C(=O)O]

4-(Azidomethyl)-5-(3-(3-(2-(3-bromophenyl)-7,7-dimethylnon-8-yn-2-yl)-1-methyl-1H-1,2,4-triazol-5-yl)-4-fluorophenoxy)-6-fluoro-1H-indole

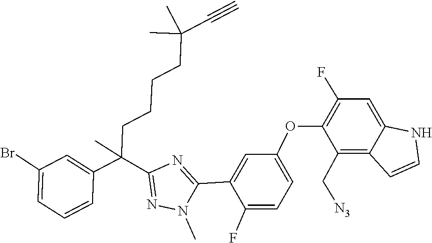 [SMILES: C#CC(C)(C)CCCCC(C)(c1cccc(Br)c1)c1nc(-c2cc(Oc3c(F)cc4[nH]ccc4c3CN=[N+]=[N-])ccc2F)n(C)n1]

Step A: Exchanging 2-(3-bromophenyl)-5-(but-2-yn-1-yloxy)-N′,2-dimethylpentanehydrazide (Intermediate 83-1) with 2-(3-bromophenyl)-N′,2,7,7-tetramethylnon-8-ynehydrazide (Intermediate 83-3, 5.3 g, 14 mmol), the reaction procedure sequence (Steps A to E) described for Example 93A, 93B was used to prepare the title compound (1.2 g). MS (ESI): 686, 688 m/z [M+H]⁺, retention time: 2.29 minutes (LC-MS Method 2).

Compounds 96A, 96B. Enantiomers 1 and 2 of 3-[3-(24,30-difluoro-3,6,11,11-tetramethyl-26-oxa-3,4,13,14,15,21,33-heptazahexacyclo-[25.3.1.12,5.112,15.017,25.018,22]tritriaconta-1(31),2(33),4,12(32),13,17,19,22,24,27,29-undecaen-6-yl)phenyl]-2-methyl-propanoic acid

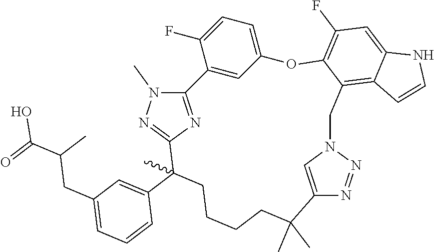 [SMILES: CC(Cc1cccc(C2(C)CCCCC(C)(C)c3cn(nn3)Cc3c(c(F)cc4[nH]ccc34)Oc3ccc(F)c(c3)-c3nc2nn3C)c1)C(=O)O]

Step B: Exchanging 4-(azidomethyl)-6-fluoro-5-(4-fluoro-3-(5-(2-(3-iodophenyl)oct-7-yn-2-yl)-1H-imidazol-2-yl)phenoxy)-1H-indole (Step A product of Example 2) with 4-(azidomethyl)-5-(3-(3-(2-(3-bromophenyl)-7,7-dimethylnon-8-yn-2-yl)-1-methyl-1H-1,2,4-triazol-5-yl)-4-fluorophenoxy)-6-fluoro-1H-indole (Step A product of this example, 1.5 g, 2.27 mmol) in Step B, and, the reaction procedure sequence (Steps B to E) described for Example 2 was used to prepare the title compounds, with modification on corresponding Step C. The Heck reaction was replaced by a Suzuki coupling under the following condition: the mixture of corresponding Step B product of Example 2 (580 mg, 0.845 mmol), ethyl (E)-2-methyl-3-(4,4,5,5-tetramethyl-1,3,2-dioxaborolan-2-yl)acrylate (304 mg, 1.27 mmol), cesium carbonate (550 mg, 1.69 mmol), Pd(dppf)Cl₂ (69 mg, 0.085 mmol) in 1.4-dioxane-water (4:1, 10 mL) was heated to 100° C. over 3 hours to give the methyl acrylate intermediate, which, after normal workup, was carried on for corresponding Step D and E of Example 2.

The racemic ethyl ester (500 mg), obtained from corresponding Step D of Example 2, was subject to SFC chiral separation using instrument: SFC-80 (Thar, Waters) under the following condition: Column: WHELK 20*250 mm, 10 μm; Column temperature: 35° C.; Mobile phase: carbon dioxide/isopropanol (0.2% methanol ammonia)=50/50; Flow rate: 80 g/minute; Back pressure: 100 bar; Detection wavelength: 214 nm; Cycle time: 3.5 minutes; Sample solution: 500 mg dissolved in 30 mL methanol; Injection volume: 0.6 mL. The first eluent, Enantiomer 1 (200 mg, 40%), was further hydrolyzed to Compound 96A (173 mg, 90%); The second eluent, Enantiomer 2 (200 mg, 40%), was further hydrolyzed to Compound 96B (164 mg, 85%). Compound 96A: MS (ESI): 694 m/z [M+H]⁺, retention time: 1.90 minutes, purity: 99% (214 nm) (LC-MS Method 2). ¹H NMR (400 MHz, CD₃OD) δ 7.49-7.43 (m, 2H), 7.41-7.33 (m, 2H), 7.30 (dd, J=10.8, 2.0 Hz, 1H), 7.13 (t, J=7.6 Hz, 1H), 7.06-6.92 (m, 3H), 6.69-6.63 (m, 1H), 6.55 (t, J=3.2 Hz, 1H), 5.99 (d, J=14.8, 1H), 5.78 (d, J=14.8, 1H), 3.85 (d, J=2.8 Hz, 3H), 2.98-2.89 (m, 1H), 2.65-2.53 (m, 2H), 2.19-2.10 (m, 1H), 2.04-1.96 (m, 1H), 1.62 (s, 3H), 1.59-1.25 (m, 3H), 1.20 (s, 3H), 1.14 (s, 3H), 1.09-1.03 (m, 3H), 1.00-0.90 (m, 2H), 0.76-0.66 (m, 1H) ppm.

Compound 96B: MS (ESI): 694 m/z [M+H]⁺, retention time: 1.90 minutes, purity: 99% (214 nm) (LC-MS Method 2). ¹H NMR (400 MHz, CD₃OD) δ 7.49-7.43 (m, 2H), 7.41-7.33 (m, 2H), 7.30 (dd, J=10.8, 2.0 Hz, 1H), 7.13 (t, J=7.6 Hz, 1H), 7.06-6.92 (m, 3H), 6.69-6.62 (m, 1H), 6.55 (t, J=3.2 Hz, 1H), 5.99 (d, J=14.8, 1H), 5.78 (d, J=14.8, 1H), 3.85 (d, J=2.8 Hz, 3H), 2.98-2.89 (m, 1H), 2.65-2.53 (m, 2H), 2.19-2.10 (m, 1H), 2.04-1.96 (m, 1H), 1.62 (s, 3H), 1.59-1.25 (m, 3H), 1.20 (s, 3H), 1.14 (s, 3H), 1.09-1.03 (m, 3H), 1.00-0.90 (m, 2H), 0.76-0.66 (m, 1H) ppm.

Example 97. Compound 97A and Compound 97B. Diastereomers 1 and 2 of 3-[3-(24,30-difluoro-3,6,11,11-tetramethyl-10,26-dioxa-3,4,13,14,15,21,33-heptazahexacyclo[25.3.1.12,5.112,15.017,25.018,22]tritriaconta-1(31),2(33),4,12(32),13,17,19,22,24,27,29-undecaen-6-yl)phenyl]-2-methyl-propanoic acid

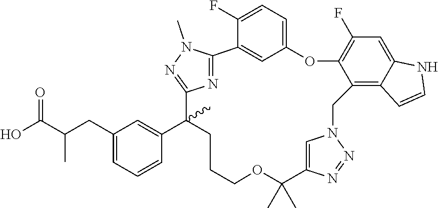 [SMILES: CC(Cc1cccc(C2(C)CCCOC(C)(C)c3cn(nn3)Cc3c(c(F)cc4[nH]ccc34)Oc3ccc(F)c(c3)-c3nc2nn3C)c1)C(=O)O]

(5-(3-(3-(2-(3-Bromophenyl)-5-((2-methylbut-3-yn-2-yl)oxy)pentan-2-yl)-1-methyl-1H-1,2,4-triazol-5-yl)-4-fluorophenoxy)-6-fluoro-1-(phenylsulfonyl)-1H-indol-4-yl)methyl acetate

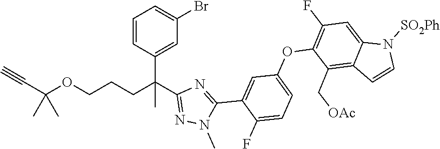

Step A: To a stirred solution of (6-fluoro-5-(4-fluoro-3-(imino(methylthio)methyl)phenoxy)-1-(phenylsulfonyl)-1H-indol-4-yl)methyl acetate hydroiodide (Intermediate 82-3, 5.29 g, 10 mmol) in pyridine (50 mL) was added 2-(3-bromophenyl)-N′,2-dimethyl-5-((2-methylbut-3-yn-2-yl)oxy)pentanehydrazide (Intermediate 83-4, 3.8 g, 10 mmol). The mixture was stirred at 75° C. for 16 hours, cooled to room temperature, and partitioned between water (150 mL) and ethyl acetate (100 mL). The separated organic phase, combined with additional ethyl acetate extracts (100 mL), was washed with hydrochloric acid (1 M), brine, dried over sodium sulfate, and concentrated to afford the crude title compound (8.0 g, crude) as a solid. MS (ESI): 845, 847 m/z [M+H]⁺, retention time: 2.49 minutes, purity: 90% (214 nm) (LC-MS Method 5)

(5-(3-(3-(2-(3-Bromophenyl)-5-((2-methylbut-3-yn-2-yl)oxy)pentan-2-yl)-1-methyl-1H-1,2,4-triazol-5-yl)-4-fluorophenoxy)-6-fluoro-1H-indol-4-yl)methanol

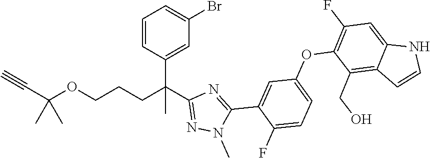 [SMILES: C#CC(C)(C)OCCCC(C)(c1cccc(Br)c1)c1nc(-c2cc(Oc3c(F)cc4[nH]ccc4c3CO)ccc2F)n(C)n1]

Step B: To a stirred solution of Step A product (8.0 g, crude) was added 0.2 M lithium hydroxide (189 mL, in methanol: water tetrahydrofuran=1:1:3). The mixture was stirred at 40° C. for 16 hours, cooled to room temperature, acidified with 40 mL of 1 N hydrochloric acid. The mixture was extracted with ethyl acetate (2×50 mL). The combined organic phases were washed with brine, dried over sodium sulfate, and concentrated. The residue was purified by silica gel chromatography to give the title compound (3.8 g, 60% 2 step). MS (ESI): 663, 665 m/z [M+H]⁺, retention time: 2.24 minutes, purity: >99% (214 nm) (LC-MS Method 5).

4-(Azidomethyl)-5-(3-(3-(2-(3-bromophenyl)-5-((2-methylbut-3-yn-2-yl)oxy)pentan-2-yl)-1-methyl-1H-1,2,4-triazol-5-yl)-4-fluorophenoxy)-6-fluoro-1H-indole

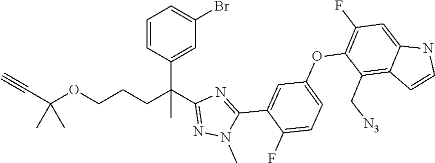 [SMILES: C#CC(C)(C)OCCCC(C)(c1cccc(Br)c1)c1nc(-c2cc(Oc3c(F)cc4[nH]ccc4c3CN=[N+]=[N-])ccc2F)n(C)n1]

Step C: To a stirred solution of Step B product (2.0 g, 3.0 mmol) in tetrahydrofuran (40 mL) was added 1,8-diazabicyclo[5.4.0]undec-7-ene (6 mmol, 918 mg) and diphenylphosphoryl azide (2.49 g, 9 mmol). The mixture was heated to reflux for 16 hours, cooled to room temperature, and quenched with saturated sodium bicarbonate (10 mL). The mixture was extracted with ethyl acetate (3×50 mL). The combined organic phases were washed with brine (10 mL), dried over sodium sulfate, and concentrated. The residue was purified by flash chromatography to afford the title compound (1.6 g, 78%) as a solid. MS (ESI): 688, 690 m/z [M+H]⁺, retention time: 2.40 minutes, purity: 60% (214 nm) (LC-MS Method 5).

Compounds 97A and 97B. Diastereomers 1 and 2 of 3-[3-(24,30-difluoro-3,6,11,11-tetramethyl-10,26-dioxa-3,4,13,14,15,21,33-heptazahexacyclo-[25.3.1.12,5.112,15.017,25.018,22]tritriaconta-1(31),2(33),4,12(32),13,17,19,22,24,27,29-undecaen-6-yl)phenyl]-2-methyl-propanoic acid

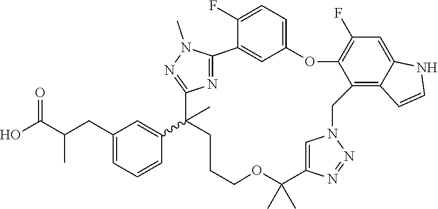 [SMILES: CC(Cc1cccc(C2(C)CCCOC(C)(C)c3cn(nn3)Cc3c(c(F)cc4[nH]ccc34)Oc3ccc(F)c(c3)-c3nc2nn3C)c1)C(=O)O]

Step D: Exchanging 4-(azidomethyl)-5-(3-(3-(2-(3-bromophenyl)-7,7-dimethylnon-8-yn-2-yl)-1-methyl-1H-1,2,4-triazol-5-yl)-4-fluorophenoxy)-6-fluoro-1H-indole (Step A product of Example % A, % B) with 4-(azidomethyl)-5-(3-(3-(2-(3-bromophenyl)-5-((2-methylbut-3-yn-2-yl)oxy)pentan-2-yl)-1-methyl-1H-1,2,4-triazol-5-yl)-4-fluorophenoxy)-6-fluoro-1H-indole (Step C product of this Example, 1.6 g, 2.32 mmol), the reaction procedure sequence (Step B) was used to prepare the title compounds. The racemic acid (700 mg), obtained at the last step, was subject to chiral SFC separation using SFC-80 (Thar, Waters) under the following condition: Column: AD 20*250 mm, 10 μm; Column temperature: 35° C.; Mobile phase: carbon dioxide/isopropanol (0.2% methanol ammonia)=70/30; Flow rate: 80 g/minute; Back pressure: 100 bar; Detection wavelength: 214 nm; Cycle time: 6.0 minutes; Sample solution: 800 mg dissolved in 25 mL of methanol; Injection volume: 1.0 mL. The first eluent, Diastereomer 1, was designated as Compound 97A (198.2 mg, 25%); The second eluent, Diastereomer 2, was designated as Compound 97B (256.4 mg, 32%).

Compound 97A: MS (ESI): 6% m/z [M+H]⁺, retention time: 2.05 minutes, purity: 95% (214 nm) (LC-MS Method 5). ¹H NMR (400 MHz, CD₃OD) δ 7.53-7.48 (m, 2H), 7.33 (t, J=9.2 Hz, 1H), 7.25-7.18 (m, 2H), 7.07 (t, J=7.6 Hz, 1H), 7.00-6.92 (m, 3H), 6.75-6.71 (m, 1H), 6.16-6.14 (m, 1H), 5.92 (d, J=14.8 Hz, 1H), 5.75 (d, J=14.8 Hz, 1H), 3.80 (d, J=1.6 Hz, 3H), 3.15-3.11 (m, 1H), 2.92-2.86 (m, 2H), 2.61-2.50 (m, 2H), 2.29-2.18 (m, 1H), 1.99-1.85 (m, 1H), 1.61 (s, 3H), 1.57-1.49 (m, 1H), 1.46 (s, 3H), 1.38 (s, 3H), 1.28-1.22 (m, 1H), 1.03-0.% (m, 3H) ppm.

Compound 97B: MS (ESI): 6% m/z [M+H]⁺, retention time: 2.05 minutes, purity: 95% (214 nm) (LC-MS Method 5). ¹H NMR (400 MHz, CD₃OD) δ 7.53-7.48 (m, 2H), 7.33 (t, J=9.2 Hz, 1H), 7.24-7.18 (m, 2H), 7.07 (t, J=7.6 Hz, 1H), 7.00-6.94 (m, 3H), 6.75-6.71 (m, 1H), 6.16-6.14 (m, 1H), 5.92 (d, J=14.8 Hz, 1H), 5.75 (d, J=14.8 Hz, 1H), 3.80 (d, J=1.6 Hz, 3H), 3.15-3.11 (m, 1H), 2.92-2.86 (m, 2H), 2.61-2.50 (m, 2H), 2.29-2.18 (m, 1H), 1.99-1.85 (m, 1H), 1.61 (s, 3H), 1.57-1.49 (m, 1H), 1.46 (s, 3H), 1.38 (s, 3H), 1.28-1.22 (m, 1H), 1.03-0.% (m, 3H) ppm.

Example 98. Compound 98A and Compound 98B. Diastereomers 1 and 2 of Regio-isomer 1, 3-[3-(24,30-difluoro-3,6,9,9-tetramethyl-11,26-dioxa-3,4,15,21,32,33-hexazahexacyclo-[25.3.1.12,5.112,15.017,25.018,22]tritriaconta-1(31),2(33),4,12(32),13,17,19,22,24,27,29-undecaen-6-yl)phenyl]-2-methyl-propanoic acid

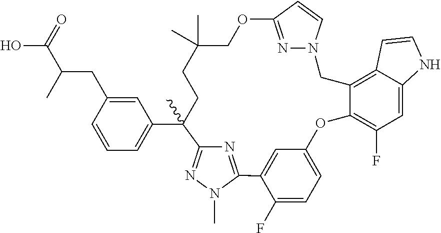 [SMILES: CC(Cc1cccc(C2(C)CCC(C)(C)COc3ccn(n3)Cc3c(c(F)cc4[nH]ccc34)Oc3ccc(F)c(c3)-c3nc2nn3C)c1)C(=O)O]

and

Example 99. Compound 99A and Compound 99B. Diastereomer 3 and 4 of Regio-isomer 2, 3-[3-(25,31-difluoro-3,6,9,9-tetramethyl-11,27-dioxa-3,4,15,16,22,33-hexazahexacyclo-[26.3.1.12,5.012,16.018,26.019,23]tritriaconta-1(32),2(33),4,12,14,18,20,23,25,28,30-undecaen-6-yl)phenyl]-2-methyl-propanoic acid

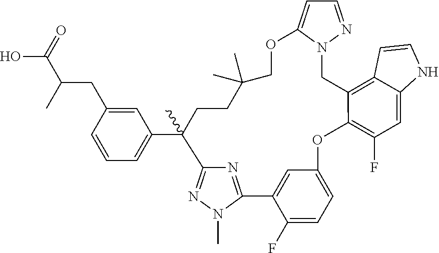 [SMILES: CC(Cc1cccc(C2(C)CCC(C)(C)COc3ccnn3Cc3c(c(F)cc4[nH]ccc34)Oc3ccc(F)c(c3)-c3nc2nn3C)c1)C(=O)O]

Ethyl 3-(3-(6-((1H-pyrazol-3-yl)oxy)-2-(5-(5-((4-(acetoxymethyl)-6-fluoro-1-(phenylsulfonyl)-1H-indol-5-yl)oxy)-2-fluorophenyl)-1-methyl-1H-1,2,4-triazol-3-yl)-5,5-dimethylhexan-2-yl)phenyl)-2-methylpropanoate

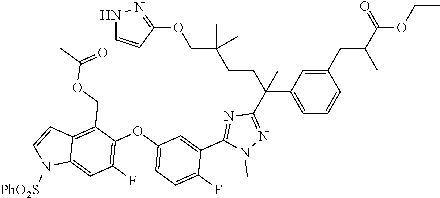

Step A: To a mixture of ethyl 3-(3-(6-((1H-pyrazol-3-yl)oxy)-2,5,5-trimethyl-1-(2-methylhydrazineyl)-1-oxohexan-2-yl)phenyl)-2-methylpropanoate (Intermediate 83-5, 1.7 g, 3.71 mmol) in pyridine (20 mL) was added (6-fluoro-5-(4-fluoro-3-(imino(methylthio)methyl)-phenoxy)-1-(phenylsulfonyl)-1H-indol-4-yl)methyl acetate hydroiodide (Intermediate 82-3, 2.44 g, 3.71 mmol). The mixture was stirred at 80° C. for 5 hours, cooled to room temperature, and quenched with water (80 mL). The solution was extracted with ethyl acetate (2×60 mL). The combined organic phases were washed with hydrochloric acid solution (1 M), brine, dried over sodium sulfate, and concentrated. The residue was purified by silica gel column chromatography (40 g silica gel column, eluting with 0-60% ethyl acetate in petroleum ether) to give the title compound (1.8 g, 53%) as a solid. MS (ESI): 923 m/z [M+H]⁺, retention time: 2.10 minutes, purity: 85% (214 nm) (LC-MS Method 2).

Ethyl 3-(3-(6-((1H-pyrazol-3-yl)oxy)-2-(5-(2-fluoro-5-((6-fluoro-4-(hydroxymethyl)-1-(phenylsulfonyl)-1H-indol-5-yl)oxy)phenyl)-1-methyl-1H-1,2,4-triazol-3-yl)-5,5-dimethylhexan-2-yl)phenyl)-2-methylpropanoate

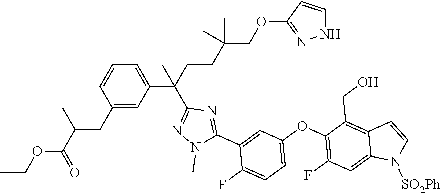

Step B: To a stirred solution of Step A product (1.8 g, 1.95 mmol) in ethanol (20 mL) was added potassium carbonate (0.54 g, 3.9 mmol). The mixture was stirred at room temperature for two hours, quenched with water (80 mL), and extracted with ethyl acetate (2×60 mL). The combined organic phases were washed with brine, dried over sodium sulfate, and concentrated to give the crude title compound (1.6 g, 93%) as a solid, which was used for next step without further purification. MS (ESI): 881 m/z [M+H]⁺, retention time: 2.04 minutes, purity: 85% (214 nm) (LC-MS Method 2).

Regio Isomer 1 of ethyl 3-[3-[21-(benzenesulfonyl)-24,30-difluoro-3,6,9,9-tetramethyl-11,26-dioxa-3,4,15,21,32,33-hexazahexacyclo[25.3.1.12,5.112,15.017,25.018,22]-tritriaconta-1(31),2(33),4,12(32),13,17,19,22,24,27,29-undecaen-6-yl]phenyl]-2-methyl-propanoate

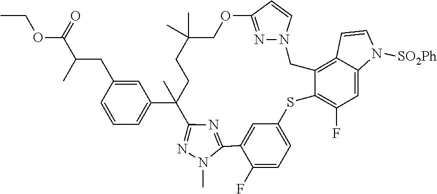

Regio isomer 2 of ethyl 3-[3-[22-(benzenesulfonyl)-25,31-difluoro-3,6,9,9-tetramethyl-11,27-dioxa-3,4,15,16,22,33-hexazahexacyclo[26.3.1.12,5.012,16.018,26.019,23]tritriaconta-1(32),2(33),4,12,14,18,20,23,25,28,30-undecaen-6-yl]phenyl]-2-methyl-propanoate

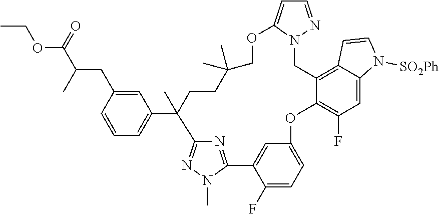

Step C: To a stirred solution of Step B product (1.2 g, 1.36 mmol) in toluene (100 mL) was added cyanomethylenetributylphosphorane (1.64 g, 6.81 mmol). The mixture was stirred at 150° C. for 1 hour in a microwave reactor. The solvent was removed. The residue was purified by silica gel column chromatography (25 g silica gel column, eluting with 0-50% ethyl acetate in petroleum ether). The first peak is regioisomer 1 (250 mg, 21%) and the second peak is regioisomer 2, (250 mg, 21%).

Regioisomer 1: MS (ESI): 863 m/z [M+H]⁺, retention time: 2.26 minutes, purity: 95% (214 nm) (LC-MS Method 2).

Regioisomer 2: MS (ESI): 863 m/z [M+H]⁺, retention time: 2.23 minutes, purity: 95% (214 nm) (LC-MS Method 2).

Diastereomers 1 and 2 of Regio-isomer 1, ethyl 3-[3-[21-(benzenesulfonyl)-24,30-difluoro-3,6,9,9-tetramethyl-11,26-dioxa-3,4,15,21,32,33-hexazahexacyclo[25.3.1.12,5.112,15.017,25.018,22]-tritriaconta-1(31),2(33),4,12(32),13,17,19,22,24,27,29-undecaen-6-yl]phenyl]-2-methyl-propanoate

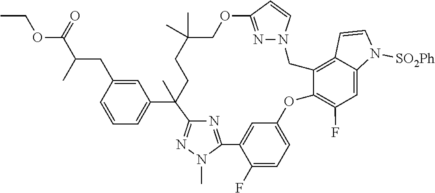

and Diastereomer 3 and 4 of Regio-isomer 2, ethyl 3-[3-[22-(benzenesulfonyl)-25,31-difluoro-3,6,9,9-tetramethyl-11,27-dioxa-3,4,15,16,22,33-hexazahexacyclo[26.3.1.12,5.012,16.018,26.019,23]tritriaconta-1(32),2(33),4,12,14,18,20,23,25,28,30-undecaen-6-yl]phenyl]-2-methyl-propanoate

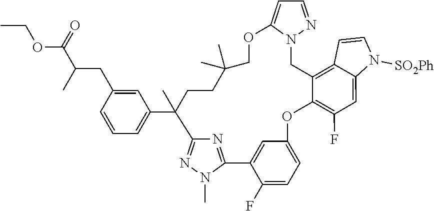

Step D: The racemic regioisomer 1 of Step C product (290 mg, 0.336 mmol) was subjected to chiral SFC separating using SFC-150 (Waters) under the following condition: Column: (S,S)-Whelk-O1 20×250 mm, 10 μm (Regis); Column temperature: 35° C.; Mobile phase: carbon dioxide/(methanol: acetonitrile=1:1 (0.2% methanol ammonia))=40/60; Flow rate: 120 g/minute; Back pressure: 100 bar; Detection wavelength: 214 nm; Cycle time: 2.3 minutes; Injection volume: 1.9 mL (20 injections). The first eluent was designated as Diastereomer 1 (125 mg, 43%); The second eluent was designated as Diastereomer 2 (125 mg, 43%).

The racemic Regioisomer 2 of Step C product (290 mg, 0.336 mmol) was subjected to chiral prep-HPLC separation using Gilson-281 under the following condition: Column: (R,R)-Whelk-O1 20×250 mm, 10 μm; Column temperature: 35° C.; Mobile phase: n-hexane (0.1% diethylamine): Ethanol (0.1% diethylamine)=40:60; Flow rate: 50 mL/minute; Detection wavelength: 214 nm; Cycle time: 22 minutes; Injection volume: 0.5 mL (20 injections). The first eluent was designated as Diastereomer 3 (100 mg, 35%); The second eluent was designated as Diastereomer 4 (100 mg, 35%).

Diastereomer 1 of Regioisomer 1: MS (ESI): 863 m/z [M+H]⁺, retention time: 2.25 minutes, purity: 96% (214 nm) (LC-MS Method 2).

Diastereomer 2 of Regioisomer 1: MS (ESI): 863 m/z [M+H]⁺, retention time: 2.25 minutes, purity: 99% (214 nm) (LC-MS Method 2).

Diastereomer 3 of Regioisomer 2: MS (ESI): 863 m/z [M+H]⁺, retention time: 2.20 minutes, purity: 96% (214 nm) (LC-MS Method 2).

Diastereomer 4 of Regioisomer 2: MS (ESI): 863 m/z [M+H]⁺, retention time: 2.20 minutes, purity: 99% (214 nm) (LC-MS Method 2).

Compounds 98A, 98B, Diastereomers 1 and 2 of Regio-isomer 1, 3-[3-(24,30-difluoro-3,6,9,9-tetramethyl-11,26-dioxa-3,4,15,21,32,33-hexazahexacyclo-125.3.1.12,5.112,15.017,25.018,22]tritriaconta-1(31),2(33),4,12(32),13,17,19,22,24,27,29-undecaen-6-yl)phenyl]-2-methyl-propanoic acid

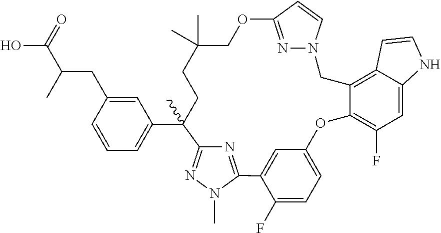 [SMILES: CC(Cc1cccc(C2(C)CCC(C)(C)COc3ccn(n3)Cc3c(c(F)cc4[nH]ccc34)Oc3ccc(F)c(c3)-c3nc2nn3C)c1)C(=O)O]

Compound 99A & 99B. Diastereomer 3 and 4 of Regio-isomer 2, 3-[3-(25,31-difluoro-3,6,9,9-tetramethyl-11,27-dioxa-3,4,15,16,22,33-hexazahexacyclo-[26.3.1.12,5.012,16.018,26.019,23]tritriaconta-1(32),2(33),4,12,14,18,20,23,25,28,30-undecaen-6-yl)phenyl]-2-methyl-propanoic acid

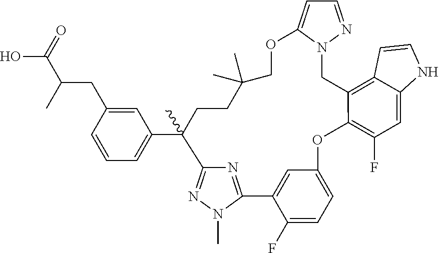 [SMILES: CC(Cc1cccc(C2(C)CCC(C)(C)COc3ccnn3Cc3c(c(F)cc4[nH]ccc34)Oc3ccc(F)c(c3)-c3nc2nn3C)c1)C(=O)O]

Step E: To a stirred solution of Diastereomer 1 of Regioisomer 1 of Step D product (125 mg, 0.145 mmol) in tetrahydrofuran (1.5 mL) and methanol (0.5 mL) was added lithium hydroxide monohydrate (18.2 mg, 0.435 mmol). The mixture was stirred at room temperature for 5 hours, diluted with water, and acidified to pH ˜5 with 1 N hydrochloric acid. The mixture was extracted with ethyl acetate (2×20 mL). The combined organic phases were washed with water, brine, dried over sodium sulfate, and concentrated to give Compound 98A (95 mg, 94%) as a white solid.

An identical procedure from above was used to prepare Compound 98B (98.6 mg, 98%) from Diastereomer 2 of Regioisomer 1 (125 mg, 0.145 mmol), Compound 99A (73 mg, 91%) from Diastereomer 3 of Regioisomer 2 (100 mg, 0.116 mmol), and Compound 99B (75 mg, 93%) from Diastereomer 4 of Regioisomer 2 (100 mg, 0.116 mmol).

Compound 98A: MS (ESI): 695 m/z [M+H]⁺, retention time: 1.96 minutes, purity: 99% (214 nm) (LC-MS Method 2). ¹H NMR (400 MHz, CD₃OD) δ 7.39-7.33 (m, 2H), 7.28-7.19 (m, 3H), 7.18-7.13 (m, 1H), 7.09-7.04 (m, 1H), 7.04-6.97 (m, 2H), 6.67 (d, J=3.2 Hz, 1H), 6.23-6.19 (m, 1H), 5.48-5.35 (m, 2H), 5.33 (d, J=1.6 Hz, 1H), 3.73 (dd, J=9.2, 3.2 Hz, 1H), 3.66 (s, 3H), 3.25 (d, J=9.2 Hz, 1H), 2.98-2.91 (m, 1H), 2.63-2.54 (m, 2H), 2.19-2.03 (m, 2H), 1.58 (s, 3H), 1.27-1.18 (m, 2H), 1.08-1.05 (m, 3H), 0.95 (s, 3H), 0.89 (s, 3H) ppm.

Compound 98B: MS (ESI): 695 m/z [M+H]⁺, retention time: 1.96 minutes, purity: 99% (214 nm) (LC-MS Method 2). ¹H NMR (400 MHz, CD₃OD) δ 7.39-7.33 (m, 2H), 7.28-7.19 (m, 3H), 7.18-7.13 (m, 1H), 7.09-7.04 (m, 1H), 7.04-6.97 (m, 2H), 6.67 (d, J=3.2 Hz, 1H), 6.23-6.19 (m, 1H), 5.48-5.35 (m, 2H), 5.34 (d, J=1.6 Hz, 1H), 3.73 (dd, J=9.2, 3.2 Hz, 1H), 3.66 (s, 3H), 3.25 (d, J=9.2 Hz, 1H), 2.98-2.91 (m, 1H), 2.63-2.54 (m, 2H), 2.19-2.03 (m, 2H), 1.58 (s, 3H), 1.27-1.18 (m, 2H), 1.08-1.05 (m, 3H), 0.95 (s, 3H), 0.89 (s, 3H) ppm.

Compound 99A: MS (ESI): 695 m/z [M+H]⁺, retention time: 1.88 minutes, purity: 97% (214 nm) (LC-MS Method 2). ¹H NMR (400 MHz, CD₃OD) δ 7.53-7.48 (m, 1H), 7.38 (t, J=9.2 Hz, 1H), 7.24-7.12 (m, 4H), 7.05-6.93 (m, 3H), 6.60-6.54 (m, 1H), 5.68-5.56 (m, 3H), 5.55-5.49 (m, 1H), 3.83 (d, J=2.4 Hz, 3H), 3.76-3.64 (m, 2H), 2.96-2.89 (m, 1H), 2.65-2.56 (m, 2H), 2.20-2.13 (m, 1H), 1.83-1.75 (m, 1H), 1.64 (s, 3H), 1.41-1.34 (m, 2H), 1.33-1.25 (m, 6H), 1.10-1.06 (m, 3H) ppm.

Compound 99B: MS (ESI): 695 m/z [M+H]⁺, retention time: 1.88 minutes, purity: 96% (214 nm) (LC-MS Method 2). ¹H NMR (400 MHz, CD₃OD) δ 7.53-7.48 (m, 1H), 7.38 (t, J=9.2 Hz, 1H), 7.24-7.12 (m, 4H), 7.05-6.93 (m, 3H), 6.60-6.54 (m, 1H), 5.68-5.56 (m, 3H), 5.55-5.49 (m, 1H), 3.83 (d, J=2.4 Hz, 3H), 3.76-3.64 (m, 2H), 2.96-2.89 (m, 1H), 2.65-2.56 (m, 2H), 2.20-2.13 (m, 1H), 1.83-1.75 (m, 1H), 1.64 (s, 3H), 1.41-1.34 (m, 2H), 1.33-1.25 (m, 6H), 1.10-1.06 (m, 3H) ppm.

Example 100. Compound 100A and Compound 100B. 3-[3-[(5S)-24-Fluoro-11,11-dimethyl-10,26-dioxa-5,13,14,15,21,33-hexazahexacyclo[25.3.1.12,5.112,15.017,25.018,22]tritriaconta-1(31),2(33),3,12(32),13,17,19,22,24,27,29-undecaen-6-yl]phenyl]-2-methyl-propanoic acid

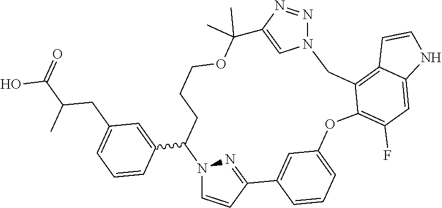

(5-(3-(1-(1-(3-Bromophenyl)-4-((2-methylbut-3-yn-2-yl)oxy)butyl)-1H-pyrazol-3-yl)phenoxy)-6-fluoro-1-tosyl-1H-indol-4-yl)methanol

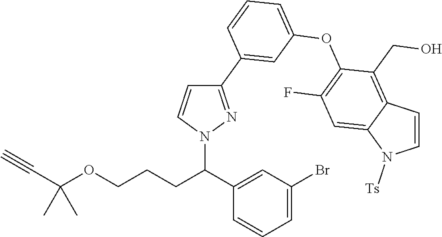 [SMILES: C#CC(C)(C)OCCCC(c1cccc(Br)c1)n1ccc(-c2cccc(Oc3c(F)cc4c(ccn4S(=O)(=O)c4ccc(C)cc4)c3CO)c2)n1]

Step A: To stirred solution of (5-(3-(1H-pyrazol-5-yl)phenoxy)-6-fluoro-1-tosyl-1H-indol-4-yl)methanol (Intermediate 55-4, 2.3 g, 4.8 mmol) were added 1-bromo-3-(1-bromo-4-((2-methylbut-3-yn-2-yl)oxy)butyl)benzene (Intermediate 59, 4.8 mmol, 1.8 g), cesium carbonate (9.6 mmol, 3.14 g) in N,N-dimethylformamide (50 mL) and the mixture was stirred at room temperature for 74 hours, quenched with water (100 mL) and the aqueous phase was extracted with ethyl acetate (3×50 mL). The combined organic phases were washed with saturated lithium chloride, brine, dried over sodium sulfate, and concentrated. The crude material was purified by flash chromatography to afford the title compound (1.6 g, 45%). MS (ESI): 770,772 m/z [M+H]⁺, retention time: 2.42 minutes, purity: 96% (214 nm) (LC-MS Method 5).

(5-(3-(1-(1-(3-Bromophenyl)-4-((2-methylbut-3-yn-2-yl)oxy)butyl)-1H-pyrazol-3-yl)phenoxy)-6-fluoro-1H-indol-4-yl)methanol

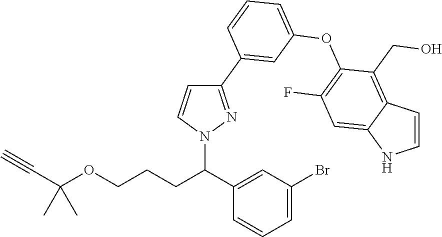 [SMILES: C#CC(C)(C)OCCCC(c1cccc(Br)c1)n1ccc(-c2cccc(Oc3c(F)cc4[nH]ccc4c3CO)c2)n1]

Step B: A mixture of Step A product (1.6 g, 2.08 mmol) and 0.2 M lithium hydroxide (in methanol: water: tetrahydrofuran=1:1:3, 41.5 mL) was stirred at room temperature for 5 hours, then acidified with 20 mL of 1 N hydrochloric acid. The mixture was extracted with ethyl acetate (2×50 mL). The combined organic phases were washed with brine, dried over sodium sulfate and concentrate. The residue was purified by chromatography to afford the title compound (1.3 g, 95%). MS (ESI): 616, 618 m/z [M+H]⁺, retention time: 2.25 minutes, purity: 90% (254 nm) (LC-MS Method 5).

Example 100A and 100B. 3-[3-[(5S)-24-Fluoro-11,11-dimethyl-10,26-dioxa-5,13,14,15,21,33-hexazahexacyclo[25.3.1.12,5.112,15.017,25.018,22]tritriaconta-1(31),2(33),3,12(32),13,17,19,22,24,27,29-undecaen-6-yl]phenyl]-2-methyl-propanoic acid

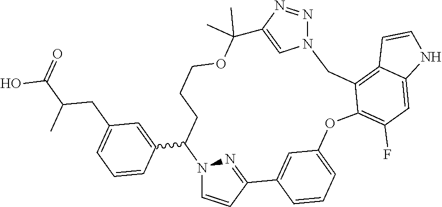

Step C: Exchanging (5-(3-(3-(2-(3-Bromophenyl)-5-((2-methylbut-3-yn-2-yl)oxy)pentan-2-yl)-1-methyl-1H-1,2,4-triazol-5-yl)-4-fluorophenoxy)-6-fluoro-1H-indol-4-yl)methanol (Step B product of Example 97A and 97B) with (5-(3-(1-(1-(3-bromophenyl)-4-((2-methylbut-3-yn-2-yl)oxy)butyl)-1H-pyrazol-3-yl)phenoxy)-6-fluoro-1H-indol-4-yl)methanol (Step B product of this example, 1.3 g, 2.11 mmol), the reaction procedure sequence (Steps C and D) described for Example 97A and 97B was used to prepare the title compounds. The racemic acid (250 mg) obtained at the last step was subject to Chiral SFC separation using SFC-80 (Thar, Waters) under the following condition: Column: Chiralpak AD 20×250 mm, 10 μm; Column temperature: 35° C.; Mobile phase: carbon dioxide/isopropanol (0.2% methanol ammonia)=60/40; Flow rate: 80 g/minute; Back pressure: 100 bar; Detection wavelength: 214 nm; Cycle time: 6 minutes; Sample solution: 250 mg dissolved in 20 mL methanol; Injection volume: 1.0 mL. The first eluent is designated as 100A (37.5 mg, 15%) and the second eluent is designated as Compound 100B (49.1 mg, 20%).

Compound 100A: MS (ESI): 649 m/z [M+H]⁺, retention time: 2.10 minutes, purity: 95% (214 nm) (LC-MS Method 5). ¹H NMR (400 MHz, CD₃OD) δ 7.54 (d, J=2.0 Hz, 1H), 7.46-7.34 (m, 4H), 7.33-7.27 (m, 2H), 7.23 (d, J=7.8 Hz, 1H), 7.19-7.01 (m, 4H), 6.59 (d, J=2.0 Hz, 1H), 6.21 (d, J=3.2 Hz, 1H), 5.97-5.85 (m, 2H), 5.19-5.16 (m, 1H), 3.25-3.18 (m, 1H), 3.05-2.90 (m, 2H), 2.71-2.45 (m, 3H), 1.84-1.74 (m, 1H), 1.54-1.44 (m, 1H), 1.41 (s, 3H), 1.33 (s, 3H), 1.27-1.19 (m, 1H), 1.03 (d, J=6.4 Hz, 3H) ppm.

Compound 100B: MS (ESI): 649 m/z [M+H]⁺, retention time: 2.10 minutes, purity: 97% (214 nm) (LC-MS Method 5). ¹H NMR (400 MHz, CD₃OD) δ 7.54 (d, J=2.0 Hz, 1H), 7.46-7.34 (m, 4H), 7.33-7.27 (m, 2H), 7.23 (d, J=7.8 Hz, 1H), 7.19-7.01 (m, 4H), 6.59 (d, J=2.0 Hz, 1H), 6.21 (d, J=3.2 Hz, 1H), 5.97-5.85 (m, 2H), 5.19-5.16 (m, 1H), 3.25-3.18 (m, 1H), 3.05-2.90 (m, 2H), 2.71-2.45 (m, 3H), 1.84-1.74 (m, 1H), 1.54-1.44 (m, 1H), 1.41 (s, 3H), 1.33 (s, 3H), 1.27-1.19 (m, 1H), 1.03 (d, J=6.4 Hz, 3H) ppm.

Example 101. Compound 101A and Compound 101B. Enantiomers 1 and 2 of 3-[3-(24-fluoro-11,11-dimethyl-10,26-dioxa-5,13,14,15,21,33-hexazahexacyclo[25.3.1.12,5.112,15.017,25.018,22]-tritriaconta-1(31),2(33),3,12(32),13,17,19,22,24,27,29-undecaen-6-yl)phenyl]propanoic acid

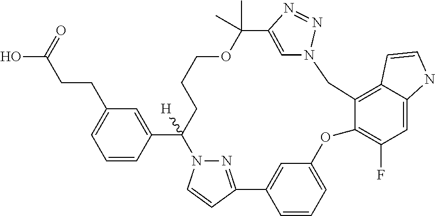 [SMILES: CC1(C)OCCCC(c2cccc(CCC(=O)O)c2)n2ccc(n2)-c2cccc(c2)Oc2c(F)cc3[nH]ccc3c2Cn2cc1nn2]

and

Example 102. Compound 102. 24-Fluoro-11,11-dimethyl-6-phenyl-10,26-dioxa-5,13,14,15,21,33-hexazahexacyclo[25.3.1.12,5.112,15.017,25.018,22]tritriaconta-1(31),2(33),3,12(32),13,17,19,22,24,27,29-undecaene

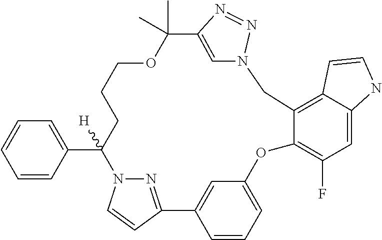 [SMILES: CC1(C)OCCCC(c2ccccc2)n2ccc(n2)-c2cccc(c2)Oc2c(F)cc3[nH]ccc3c2Cn2cc1nn2]

4-(Azidomethyl)-5-(3-(1-(1-(3-bromophenyl)-4-((2-methylbut-3-yn-2-yl)oxy)butyl)-1H-pyrazol-3-yl)phenoxy)-6-fluoro-1H-indole

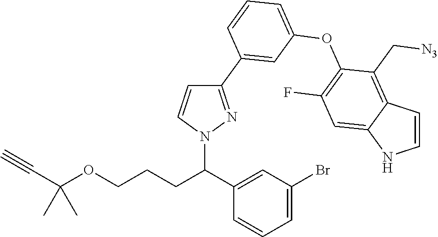 [SMILES: C#CC(C)(C)OCCCC(c1cccc(Br)c1)n1ccc(-c2cccc(Oc3c(F)cc4[nH]ccc4c3CN=[N+]=[N-])c2)n1]

Step A: To a stirred solution of (5-(3-(1-(1-(3-bromophenyl)-4-((2-methylbut-3-yn-2-yl)oxy)butyl)-1H-pyrazol-3-yl)phenoxy)-6-fluoro-1H-indol-4-yl)methanol (Step B product of Example 100A and 100B, 770 mg, 1.25 mmol) in 30 mL of tetrahydrofuran was added diphenylphosphoryl azide (1.03 g, 3.75 mmol) and DBU (0.285 g, 1.87 mmol). The reaction was refluxed for 40 hours, cooled to room temperature, and quenched with saturated ammonium chloride (50 mL). The solution was extracted with ethyl acetate (3×30 mL). The combined organic phases were washed with water, brine, dried over sodium sulfate and concentrated. The residue was purified by silica gel chromatography (eluting with hexane:ethyl acetate=30:60) to afford the title compound (500 mg, 63%). MS (ESI): 641, 643 m/z [M+H]⁺, retention time: 2.38 minutes, purity: 97% (214 nm) (LC-MS Method 5).

Example 101A, 101B. Enantiomers 1 and 2 of 3-[3-(24-fluoro-11,11-dimethyl-10,26-dioxa-5,13,14,15,21,33-hexazahexacyclo[25.3.1.12,5.112,15.017,25.018,22]-tritriaconta-1(31),2(33),3,12(32),13,17,19,22,24,27,29-undecaen-6-yl)phenyl]propanoic acid

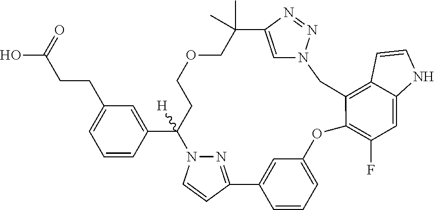 [SMILES: CC1(C)COCCC(c2cccc(CCC(=O)O)c2)n2ccc(n2)-c2cccc(c2)Oc2c(F)cc3[nH]ccc3c2Cn2cc1nn2]

and

Example 102. 24-Fluoro-11,11-dimethyl-6-phenyl-10,26-dioxa-5,13,14,15,21,33-hexazahexacyclo[25.3.1.12,5.112,15.017,25.018,22]tritriaconta-1(31),2(33),3,12(32),13,17,19,22,24,27,29-undecaene

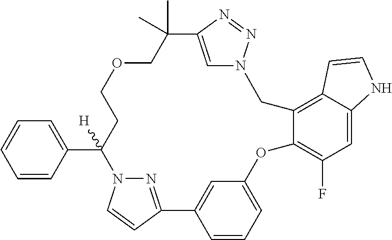 [SMILES: CC1(C)COCCC(c2ccccc2)n2ccc(n2)-c2cccc(c2)Oc2c(F)cc3[nH]ccc3c2Cn2cc1nn2]

Step B: Exchanging 4-(azidomethyl)-6-fluoro-5-(4-fluoro-3-(5-(2-(3-iodophenyl)oct-7-yn-2-yl)-1H-imidazol-2-yl)phenoxy)-1H-indole (Step A product of Example 2) with 4-(azidomethyl)-5-(3-(1-(1-(3-bromophenyl)-4-((2-methylbut-3-yn-2-yl)oxy)butyl)-1H-pyrazol-3-yl)phenoxy)-6-fluoro-1H-indole (Step A product of this example, 500 mg, 0.78 mmol), the reaction procedure sequence (Steps B to E) described for Example 2 was used to prepare the title compounds. The debromination product, Compound 102 (11 mg), originated from corresponding Step C, the Heck reaction, was separated at corresponding Step E, after hydrolysis. Also separated from corresponding Step E was the racemic Heck coupling product (150 mg). This racemic acid was subjected to Chiral SFC separation under the following conditions: Instrument: SFC-80 (Thar, Waters); Column: RRWHELK 20*250 mm, 10 μm; Column temperature: 35° C., Mobile phase: carbon dioxide/ethanol (0.5% methanol ammonia as additive)=50/50, Flow rate: 80 g/minute; Back pressure: 100 bar; Detection wavelength: 214 nm; Cycle time: 4.5 minutes, Sample solution:0.15 g dissolved in 15 mL methanol; Injection volume: 1 mL. The first eluent is Compound 101A (56 mg, 37%), the second eluent is Compound 101B (51 mg, 34%).

Compound 101A: MS (ESI): 635 m/z [M+H]⁺, retention time: 2.14 minutes, purity: 95% (254 nm) (LC-MS Method 2). 1H NMR (400 MHz, CD₃OD) δ 7.43 (s, 1H), 7.33-7.22 (m, 4H), 7.17 (d, J=10.7 Hz, 2H), 7.13-6.90 (m, 5H), 6.46 (s, 1H), 6.08 (s, 1H), 5.81 (d, J=15.0 Hz, 1H), 5.77 (d, J=15.0 Hz, 1H), 5.04 (d, J=11.2 Hz, 1H), 3.08 (m, 1H), 2.89 (m, 1H), 2.72 (t, J=7.4 Hz, 2H), 2.49-2.35 (m, 3H), 1.65 (m, 1H), 1.38 (m, 1H), 1.29 (s, 3H), 1.20 (s, 3H), 1.10 (m, 1H).

Compound 101B: MS (ESI): 635 m/z [M+H]⁺, retention time: 2.14 minutes, purity: 95% (254 nm) (LC-MS Method 2). 1H NMR (400 MHz, CD₃OD) δ 7.43 (s, 1H), 7.33-7.22 (m, 4H), 7.17 (d, J=10.7 Hz, 2H), 7.13-6.90 (m, 5H), 6.46 (s, 1H), 6.08 (s, 1H), 5.81 (d, J=15.0 Hz, 1H), 5.77 (d, J=15.0 Hz, 1H), 5.04 (d, J=11.2 Hz, 1H), 3.08 (m, 1H), 2.89 (m, 1H), 2.72 (t, J=7.4 Hz, 2H), 2.49-2.35 (m, 3H), 1.65 (m, 1H), 1.38 (m, 1H), 1.29 (s, 3H), 1.20 (s, 3H), 1.10 (m, 1H).

Compound 102: MS (ESI): 563 m/z [M+H]⁺, retention time: 2.16 minutes, purity: 95% (254 nm) (LC-MS Method 2). 1H NMR (400 MHz, CD₃OD) δ 7.58 (s, 1H), 7.42 (dd, J=17.5, 9.5 Hz, 5H), 7.32-7.20 (m, 7H), 6.60 (s, 1H), 6.24 (s, 1H), 5.92 (t, J=7.7 Hz, 2H), 5.23 (d, J=7.8 Hz, 1H), 3.19 (m, 1H), 3.01 (m, 1H), 2.55 (m, 1H), 1.83 (m, 1H), 1.53 (m, 1H), 1.41 (s, 3H), 1.33 (s, 3H), 1.26 (m, 1H).

Example 103. Compound 103A and Compound 103B. Diastereomer 1 and 2 of 3-[3-(10,16-difluoro-19,22-dimethyl-12-oxa-7,19,20,29,32-pentazahexacyclo[25.2.2.113,17.118,21.03,11.04,8]tritriaconta-1(29),3,5,8,10,13,15,17(33),18(32),20,27,30-dodecaen-22-yl)phenyl]-2-methyl-propanoic acid

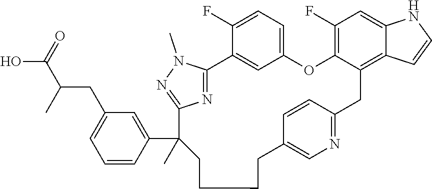 [SMILES: CC(Cc1cccc(C2(C)CCCCc3ccc(nc3)Cc3c(c(F)cc4[nH]ccc34)Oc3ccc(F)c(c3)-c3nc2nn3C)c1)C(=O)O]

Exchanging methyl 2-fluoro-5-((6-fluoro-4-((4-(2-methylpent-4-en-2-yl)-1H-pyrazol-1-yl)-methyl)-1-(phenylsulfonyl)-1H-indol-5-yl)oxy)benzimidothioate hydroiodide (Intermediate 82) with methyl 5-((4-((5-allylpyridin-2-yl)methyl)-6-fluoro-1H-indol-5-yl)oxy)-2-fluorobenzimidothioate hydroiodide (intermediate 82-4, 1 g, 2.22 mmol), the reaction procedure sequence (Steps A, B, D, E, F, G and H) described for Example 92A and 92B was used to prepare the title compounds, with modification on Step D. Instead of Heck reaction, a Suzuki coupling of corresponding Step B macrocyclization product (350 mg, 0.536 mmol) with ethyl (E)-2-methyl-3-(4,4,5,5-tetramethyl-1,3,2-dioxaborolan-2-yl)acrylate (193 mg, 0.805 mmol) (in the presence of 1,1′-bis(diphenylphosphino)ferrocene-palladium(ii)dichloride dichloromethane complex (43.8 mg, 0.0536 mmol) and cesium carbonate (350 mg, 1.07 mmol) in 1,4-dioxane (20 mL) and water (4 mL), and heated to 100° C. for 16 hours with normal workup procedure) was applied to obtain the methyl acrylate intermediate.

The corresponding Step F chiral SFC separation conditions are modified as follow: Instrument: SFC-150 (Thar, Waters); Column: R′R WHELK 20*250 mm, 10 μm; Column temperature: 35° C.; Mobile phase: carbon dioxide/methanol (0.2% methanol ammonia)=50/50; Flow rate: 120 g/minute; Back pressure: 100 bar; Detection wavelength: 214 nm; Cycle time: 4 minutes; Sample solution: 0.21 g dissolved in 40 mL methanol and dichloromethane; Injection volume:1.9 mL. The first eluent, Diastereomer 1 (85 mg, 41%), was further hydrolyzed to Compound 103A (44.8 mg, 55%); The second eluent, Diastereomer 2 (63 mg, 30%), was further hydrolyzed to Compound 103B (29.3 mg, 49%).

Compound 103A: MS (ESI): 662 m/z [M+H]⁺, retention time: 1.55 minutes, purity: >99% (214 nm) (LC-MS Method 2). ¹H NMR (400 MHz, CD₃OD) δ 8.07 (d, J=1.6 Hz, 1H), 7.48-7.44 (m, 1H), 7.34-7.29 (m, 3H), 7.24 (d, J=10.8 Hz, 1H), 7.19 (t, J=8.0 Hz, 2H), 7.04-6.97 (m, 4H), 6.58 (d, J=3.2 Hz, 1H), 6.24-6.22 (m, 1H), 4.57 (d, J=16.0 Hz, 1H), 4.36 (d, J=15.6 Hz, 1H), 3.59 (s, 3H), 2.96-2.92 (m, 1H), 2.78-2.73 (m, 1H), 2.65-2.55 (m, 2H), 2.49-2.42 (m, 1H), 2.26-2.10 (m, 1H), 1.99-1.90 (m, 1H), 1.78-1.74 (m, 1H), 1.63 (s, 3H), 1.49-1.43 (m, 1H), 1.26-1.24 (m, 1H), 1.08 (d, J=6.4 Hz, 3H), 0.68-0.64 (m, 1H) ppm.

Compound 103B: MS (ESI): 662 m/z [M+H]⁺, retention time: 1.55 minutes, purity: >99% (214 nm) (LC-MS Method 2). ¹H NMR (400 MHz, CD₃OD) δ 8.07 (d, J=1.6 Hz, 1H), 7.48-7.44 (m, 1H), 7.34-7.29 (m, 3H), 7.24 (d, J=10.8 Hz, 1H), 7.19 (t, J=8.0 Hz, 2H), 7.04-6.97 (m, 4H), 6.58 (d, J=3.2 Hz, 1H), 6.24-6.22 (m, 1H), 4.57 (d, J=16.0 Hz, 1H), 4.36 (d, J=15.6 Hz, 1H), 3.59 (s, 3H), 2.96-2.92 (m, 1H), 2.78-2.73 (m, 1H), 2.65-2.55 (m, 2H), 2.49-2.42 (m, 1H), 2.26-2.10 (m, 1H), 1.99-1.90 (m, 1H), 1.78-1.74 (m, 1H), 1.63 (s, 3H), 1.49-1.43 (m, 1H), 1.26-1.24 (m, 1H), 1.08 (d, J=6.4 Hz, 3H), 0.68-0.64 (m, 1H) ppm.

Example 104. Compound 104A and Compound 104B. Diastereomers 1 and 2 of 3-[3-(10,16-Difluoro-19,22-dimethyl-12-oxa-7,19,20,30,33-pentazahexacyclo-[26.2.2.113,17.118,21.03,11.04,8]tetratriaconta-1(30),3,5,8,10,13,15,17(34),18(33),20,28,31-dodecaen-22-yl)phenyl]-2-methyl-propanoic acid

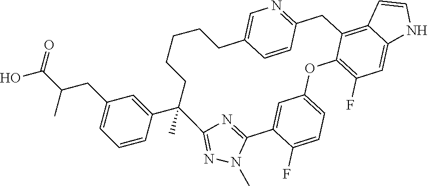 [SMILES: CC(Cc1cccc([C@@]2(C)CCCCCc3ccc(nc3)Cc3c(c(F)cc4[nH]ccc34)Oc3ccc(F)c(c3)-c3nc2nn3C)c1)C(=O)O]

Exchanging 2-(3-Bromophenyl)-N′,2-dimethylpent-4-enehydrazide (intermediate 83) with 2-(3-bromophenyl)-N′,2-dimethylhex-5-enehydrazide (Intermediate 83-6, 1.04 g, 3.34 mmol), the reaction procedure sequence (Steps A to C) described for Example 103A and 103B was used to prepare the title compounds. The chiral SFC separation condition for racemic ethyl ester was modified as follow: Instrument: SFC-80 (Thar, Waters); Column: OJ-H 20*250 mm, 10 μm; Column temperature: 35° C.; Mobile phase: carbon dioxide/methanol (0.2% methanol ammonia)=85/15; Flow rate: 70 g/minute; Back pressure: 100 bar; Detection wavelength: 214 nm; Cycle time: 12.5 minutes; Sample solution: 270 mg dissolved in 35 mL methanol; Injection volume: 1.2 mL. The first eluent (85 mg, 34%) was further hydrolyzed to Compound 104A (66.4 mg, 78%); The second eluent (88 mg, 35%), was further hydrolyzed to Compound 104B (43.1 mg, 53%).

Compound 104A: MS (ESI): 676 m/z [M+H]⁺, retention time: 1.63 minutes, purity: 98% (214 nm) (LC-MS Method 2). ¹H NMR (400 MHz, CD₃OD) δ 8.02-7.99 (m, 1H), 7.46-7.43 (m, 1H), 7.30 (d, J=3.2 Hz, 1H), 7.24-7.13 (m, 5H), 7.07-7.05 (m, 2H), 7.01 (d, J=7.6 Hz, 1H), 6.85-6.80 (m, 1H), 6.56 (d, J=2.8 Hz, 1H), 4.45 (d, J=15.6 Hz, 1H), 4.36 (d, J=15.6 Hz, 1H), 3.80 (d, J=1.6 Hz, 3H), 2.96-2.94 (m, 1H), 2.58-2.52 (m, 4H), 2.27-2.24 (m, 1H), 2.06-2.02 (m, 1H), 1.66-1.64 (m, 5H), 1.48-1.45 (m, 1H), 1.3-1.27 (m, 1H), 1.09-1.04 (m, 5H) ppm.

Compound 104B: MS (ESI): 676 m/z [M+H]⁺, retention time: 1.63 minutes, purity: >99% (214 nm) (LC-MS Method 2). ¹H NMR (400 MHz, CD₃OD) δ 8.02-7.99 (m, 1H), 7.46-7.43 (m, 1H), 7.30 (d, J=3.2 Hz, 1H), 7.24-7.13 (m, 5H), 7.07-7.05 (m, 2H), 7.01 (d, J=7.6 Hz, 1H), 6.85-6.80 (m, 1H), 6.56 (d, J=2.8 Hz, 1H), 4.46 (d, J=15.6 Hz, 1H), 4.36 (d, J=15.6 Hz, 1H), 3.80 (s, 3H), 2.96-2.94 (m, 1H), 2.58-2.52 (m, 4H), 2.27-2.24 (m, 1H), 2.06-2.02 (m, 1H), 1.66-1.64 (m, 5H), 1.48-1.45 (m, 1H), 1.3-1.27 (m, 1H), 1.09-1.04 (m, 5H) ppm.

Example 105. Compound 105A and Compound 105B. Diastereomers 1 and 2 of 3-[3-(24,30-difluoro-3,6,9,9-tetramethyl-11,26-dioxa-3,4,13,14,15,21,33-heptazahexacyclo-[25.3.1.12,5.112,15.017,25.018,22]tritriaconta-1(31),2(33),4,12(32),13,17,19,22,24,27,29-undecaen-6-yl)phenyl]-2-methyl-propanoic acid

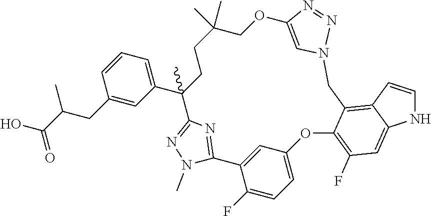 [SMILES: CC(Cc1cccc(C2(C)CCC(C)(C)COc3cn(nn3)Cc3c(c(F)cc4[nH]ccc34)Oc3ccc(F)c(c3)-c3nc2nn3C)c1)C(=O)O]

Ethyl 3-(3-(2-(5-(5-((4-((4-(benzyloxy)-1H-1,2,3-triazol-1-yl)methyl)-6-fluoro-1-(phenylsulfonyl)-1H-indol-5-yl)oxy)-2-fluorophenyl)-1-methyl-1H-1,2,4-triazol-3-yl)-6-hydroxy-5,5-dimethylhexan-2-yl)phenyl)-2-methylpropanoate

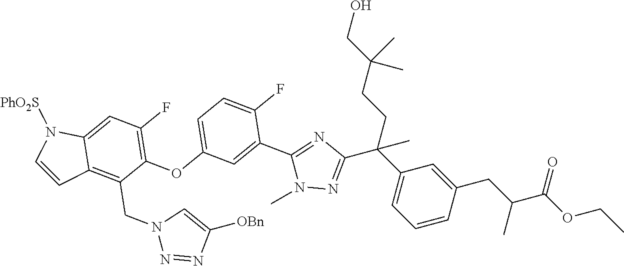

Step A: To a stirred solution of ethyl 3-(3-(6-hydroxy-2,5,5-trimethyl-1-(2-methylhydrazineyl)-1-oxohexan-2-yl)phenyl)-2-methylpropanoate (Intermediate 95, 2.2 g, 5.6 mmol) in pyridine (40 mL) was added methyl 5-((4-((4-(benzyloxy)-1H-1,2,3-triazol-1-yl)methyl)-6-fluoro-1-(phenylsulfonyl)-1H-indol-5-yl)oxy)-2-fluorobenzimidothioate hydroiodide (Intermediate 82-5, 4.34 g, 5.6 mmol). The mixture was stirred at 80° C. for 5 hours, cooled to room temperature and quenched with water (120 mL). The solution was extracted with ethyl acetate (2×80 mL). The combined organic phases were washed with hydrochloric acid (1 M), brine, dried over sodium sulfate, and concentrated. The residue was purified by silica gel column chromatography (40 g silica gel column, eluting with 0-70% ethyl acetate in petroleum ether) to give the title compound (2 g, 37%) as a solid. MS (ESI): 972 m/z [M+H]⁺, retention time: 2.15 minutes, purity: 90% (214 nm) (LC-MS Method 2).

Ethyl 3-(3-(2-(5-(2-fluoro-5-((6-fluoro-4-((4-hydroxy-1H-1,2,3-triazol-1-yl)methyl)-1-(phenylsulfonyl)-1H-indol-5-yl)oxy)phenyl)-1-methyl-1H-1,2,4-triazol-3-yl)-6-hydroxy-5,5-dimethylhexan-2-yl)phenyl)-2-methylpropanoate

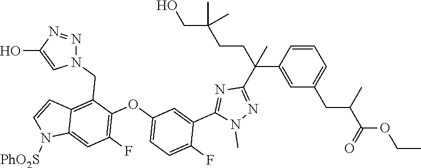

Step B: To a stirred solution of Step A product (1 g, 1.03 mmol) in tetrahydrofuran (20 mL) was added palladium on active carbon (10%, 50% wet, 0.2 g). The mixture was stirred at room temperature for 10 hours under hydrogen, then filtered through a pad of Celite. The filtrate was concentrated to give the title compound (0.85 g, yield: 94%) as a solid. MS (ESI): 882 m/z [M+H]⁺, retention time: 1.99 minutes, purity: 80% (214 nm) (LC-MS Method 2).

Ethyl 3-[3-[21-(benzenesulfonyl)-24,30-difluoro-3,6,9,9-tetramethyl-11,26-dioxa-3,4,13,14,15,21,33-heptazahexacyclo[25.3.1.12,5.112,15.017,25.018,22]tritriaconta-1(31),2(33),4,12(32),13,17,19,22,24,27,29-undecaen-6-yl]phenyl]-2-methyl-propanoate

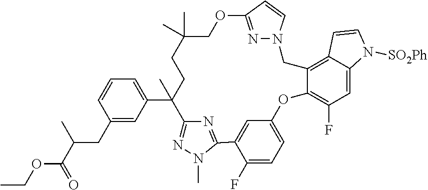

Step C: To a stirred solution of Step B product (800 mg, 0.907 mmol) in toluene (80 mL) was added cyanomethylenetributylphosphorane (1090 mg, 4.54 mmol). The mixture was stirred at 150° C. for one hour and concentrated. The residue was purified by automated silica gel column chromatography (40 g silica gel column, eluting with 0-50% ethyl acetate in petroleum ether) to give the title compound (390 mg, 50%) as a solid. MS (ESI): 864 m/z [M+H]⁺, retention time: 2.17 minutes, purity: 85% (214 nm) (LC-MS Method 2).

3-[3-(24,30-Difluoro-3,6,9,9-tetramethyl-11,26-dioxa-3,4,13,14,15,21,33-heptazahexacyclo-[25.3.1.12,5.112,15.017,25.018,22]tritriaconta-1(31),2(33),4,12(32),13,17,19,22,24,27,29-undecaen-6-yl)phenyl]-2-methyl-propanoic acid

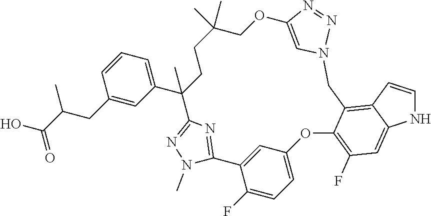 [SMILES: CC(Cc1cccc(C2(C)CCC(C)(C)COc3cn(nn3)Cc3c(c(F)cc4[nH]ccc34)Oc3ccc(F)c(c3)-c3nc2nn3C)c1)C(=O)O]

Step D: To a stirred solution of Step C product (390 mg, 0.451 mmol) in tetrahydrofuran (3 mL), methanol (1 mL) and water (1 mL) was added lithium hydroxide monohydrate (56.8 mg, 1.35 mmol). The mixture was stirred at room temperature for 5 hours, then acidified with 1 N hydrochloric acid to pH ˜5. The mixture was extracted with ethyl acetate (2×20 mL). The combined organic phases were washed with water, brine, dried over sodium sulfate, and concentrated. The residue was purified by silica gel column chromatography (40 g silica gel column, eluting with 0-10% methanol in dichloromethane) to give the title compound (260 mg, yield: 83%) as a solid. MS (ESI): 696 m/z [M+H]⁺, retention time: 1.86 minutes, purity: 93% (214 nm) (LC-MS Method 2).

Methyl 3-[3-(24,30-difluoro-3,6,9,9-tetramethyl-11,26-dioxa-3,4,13,14,15,21,33-heptazahexacyclo-[25.3.1.12,5.112,15.017,25.018,22]tritriaconta-1(31),2(33),4,12(32),13,17,19,22,24,27,29-undecaen-6-yl)phenyl]-2-methyl-propanoate

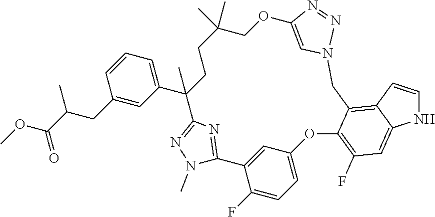 [SMILES: COC(=O)C(C)Cc1cccc(C2(C)CCC(C)(C)COc3cn(nn3)Cc3c(c(F)cc4[nH]ccc34)Oc3ccc(F)c(c3)-c3nc2nn3C)c1]

Step E: To a stirred solution of Step D product (260 mg, 0.374 mmol) in methanol (10 mL) was added concentrate sulfuric acid (0.1 mL). The mixture was stirred at 60° C. for four hours, cooled to room temperature and quenched with water (30 mL). The mixture was extracted with ethyl acetate (2×20 mL). The organic phases were washed with saturated sodium bicarbonate, brine, dried over sodium sulfate, and concentrated. The residue was purified by silica gel column chromatography (25 g silica gel column, eluting with 0-60% ethyl acetate in petroleum ether) to give the title compound (250 mg, 94%) as a solid. MS (ESI): 710 m/z [M+H]⁺, retention time: 2.00 minutes, purity: 91% (214 nm) (LC-MS Method 2).

Diastereomers 1 and 2 of Methyl 3-[3-(24,30-difluoro-3,6,9,9-tetramethyl-11,26-dioxa-3,4,13,14,15,21,33-heptazahexacyclo-[25.3.1.12,5.112,15.017,25.018,22]tritriaconta-1(31),2(33),4,12(32),13,17,19,22,24,27,29-undecaen-6-yl)phenyl]-2-methyl-propanoate

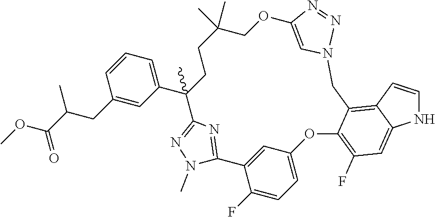 [SMILES: COC(=O)C(C)Cc1cccc(C2(C)CCC(C)(C)COc3cn(nn3)Cc3c(c(F)cc4[nH]ccc34)Oc3ccc(F)c(c3)-c3nc2nn3C)c1]

Step F: The racemic Step E product (250 mg) was subject to chiral SFC separation using SFC-80 (Thar, Waters) under following condition: Column: IC 20*250 mm, 10 μm; Column temperature: 35° C.; Mobile phase: carbon dioxide/isopropanol (0.2% methanol ammonia)=60/40; Flow rate: 80 g/minute; Back pressure: 100 bar; Detection wavelength: 214 nm; Cycle time: 5.5 minutes; Sample solution:250 mg dissolved in 25 mL of methanol; Injection volume: 1.0 mL. The first eluent is designated as Diastereomer 1 (100 mg, 40%); The second eluent is designated as Diastereomer 2 (100 mg, 40%).

Diastereomer 1: MS (ESI): 710 m/z [M+H]⁺, retention time: 2.43 minutes, purity: 96% (214 nm) (LC-MS Method).

Diastereomer 1: MS (ESI): 710 m/z [M+H]⁺, retention time: 2.43 minutes, purity: 98% (214 nm) (LC-MS Method).

Compound 105A. Diastereomers 1 of 3-[3-(24,30-difluoro-3,6,9,9-tetramethyl-11,26-dioxa-3,4,13,14,15,21,33-heptazahexacyclo-125.3.1.12,5.112,15.017,25.018,221-tritriaconta-1(31),2(33),4,12(32),13,17,19,22,24,27,29-undecaen-6-yl)phenyl]-2-methyl-propanoic acid

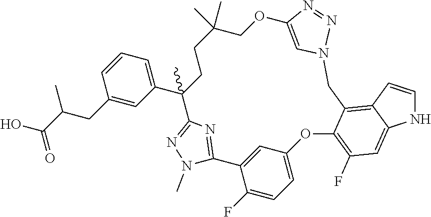 [SMILES: CC(Cc1cccc(C2(C)CCC(C)(C)COc3cn(nn3)Cc3c(c(F)cc4[nH]ccc34)Oc3ccc(F)c(c3)-c3nc2nn3C)c1)C(=O)O]

Step G: To a stirred solution of Diastereomer 1 of Step F product (100 mg, 0.141 mmol) in tetrahydrofuran (1.5 mL), methanol (0.5 mL) and water (0.5 mL) was added lithium hydroxide monohydrate (17.7 mg, 0.423 mmol). The mixture was stirred at room temperature for 5 hours, then diluted with water (20 mL) and acidified with 1 N hydrochloric acid to the pH ˜5. The mixture was extracted with ethyl acetate (2×20 mL). The combined organic phases were washed with water, brine, dried over sodium sulfate, and concentrated. The residue was purified by silica gel column chromatography (40 g silica gel column, eluting with 0-10% methanol in dichloromethane) to give the title compound (91.5 mg, 93%) as a white solid. MS (ESI): 696 m/z [M+H]⁺, retention time: 2.12 minutes, purity: 96% (214 nm) (LC-MS Method). ¹H NMR (400 MHz, CD₃OD) δ 7.51-7.42 (m, 2H), 7.38-7.29 (m, 2H), 7.12-7.07 (m, 1H), 7.05-6.88 (m, 4H), 6.78-6.76 (m, 1H), 6.01-5.89 (m, 2H), 5.75 (d, J=14.0 Hz, 1H), 3.82-3.80 (m, 3H), 3.56-3.52 (m, 1H), 3.45-3.40 (m, 1H), 2.93-2.86 (m, 1H), 2.62-2.50 (m, 2H), 2.10-1.90 (m, 2H), 1.61 (d, J=1.6 Hz, 3H), 1.35-1.25 (m, 2H), 1.03-1.00 (m, 3H), 0.97 (s, 3H), 0.91 (s, 3H) ppm.

Compound 105B. Diastereomers 2 of 3-[3-(24,30-difluoro-3,6,9,9-tetramethyl-11,26-dioxa-3,4,13,14,15,21,33-heptazahexacyclo-[25.3.1.12,5.112,15.017,25.018,22]-tritriaconta-1(31),2(33),4,12(32),13,17,19,22,24,27,29-undecaen-6-yl)phenyl]-2-methyl-propanoic acid

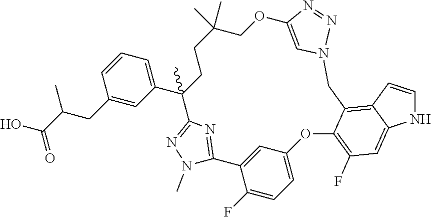 [SMILES: CC(Cc1cccc(C2(C)CCC(C)(C)COc3cn(nn3)Cc3c(c(F)cc4[nH]ccc34)Oc3ccc(F)c(c3)-c3nc2nn3C)c1)C(=O)O]

Step H: The Diastereomer 2 of Step F product (100 mg, 0.141 mmol) was subject to identical conditions as described in Step G to afford the title compound (86.5 mg, 88%) as a white solid. MS (ESI): 696 m/z [M+H]⁺, retention time: 2.12 minutes, purity: 98% (214 nm) (LC-MS Method).

¹H NMR (400 MHz, CD₃OD) δ 7.50-7.42 (m, 2H), 7.38-7.29 (m, 2H), 7.12-7.07 (m, 1H), 7.04-6.88 (m, 4H), 6.78-6.76 (m, 1H), 6.01-5.88 (m, 2H), 5.75 (d, J=14.0 Hz, 1H), 3.82-3.80 (m, 3H), 3.55-3.52 (m, 1H), 3.45-3.40 (m, 1H), 2.93-2.86 (m, 1H), 2.62-2.50 (m, 2H), 2.10-1.90 (m, 2H), 1.61 (d, J=1.6 Hz, 3H), 1.35-1.25 (m, 2H), 1.03-1.0 (m, 3H), 0.97 (s, 3H), 0.91 (s, 3H) ppm.

Example 106. Compound 106. 3-[3-(24-fluoro-3,6,11,11-tetramethyl-10,26-dioxa-3,13,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]-2-methyl-propanoic acid

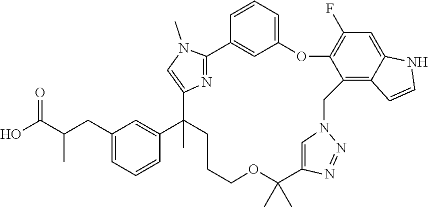 [SMILES: CC(Cc1cccc(C2(C)CCCOC(C)(C)c3cn(nn3)Cc3c(c(F)cc4[nH]ccc34)Oc3cccc(c3)-c3nc2cn3C)c1)C(=O)O]

5-(3-(4-(2-(3-Bromophenyl)-5-((2-methylbut-3-yn-2-yl)oxy)pentan-2-yl)-1-methyl-1H-imidazol-2-yl)phenoxy)-6-fluoro-4-vinyl-1H-indole

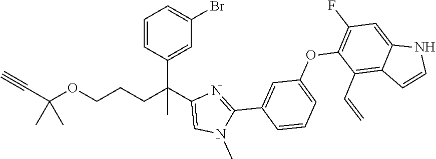 [SMILES: C#CC(C)(C)OCCCC(C)(c1cccc(Br)c1)c1cn(C)c(-c2cccc(Oc3c(F)cc4[nH]ccc4c3C=C)c2)n1]

Step A: To a stirred solution of 1-bromo-3-(3-bromophenyl)-3-methyl-6-((2-methylbut-3-yn-2-yl)oxy)hexan-2-one (Intermediate 2-48, 575 mg, 1.86 mmol) in N,N-dimethylformamide (20 mL) was added sodium bicarbonate (312 mg, 3.72 mmol) and 3-((6-fluoro-4-vinyl-1H-indol-5-yl)oxy)-N-methylbenzimidamide (Intermediate 96, 800 mg, 1.86 mmol). The mixture was heated to 75° C. and stirred for 16 hours, cooled to room temperature and quenched with water (100 mL). The mixture was extracted with ethyl acetate (2×25 mL). The combined organic phases were washed with brine (2×20 mL), dried over sodium sulfate, and concentrated. The crude material was purified by flash chromatography to afford the title compound (500 mg, 30%). MS (ESI): 640, 642 m/z [M+H]⁺, retention time: 1.87 minutes, purity: 90% (254 nm) (LC-MS Method 27).

4-(Azidomethyl)-5-(3-(4-(2-(3-bromophenyl)-5-((2-methylbut-3-yn-2-yl)oxy)pentan-2-yl)-1-methyl-1H-imidazol-2-yl)phenoxy)-6-fluoro-1H-indole

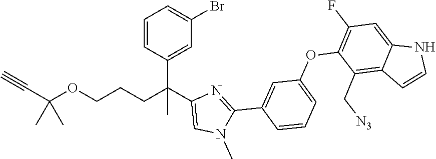 [SMILES: C#CC(C)(C)OCCCC(C)(c1cccc(Br)c1)c1cn(C)c(-c2cccc(Oc3c(F)cc4[nH]ccc4c3CN=[N+]=[N-])c2)n1]

Step B: Exchanging 5-(3-(3-(2-(3-Bromophenyl)-5-(but-2-yn-1-yloxy)pentan-2-yl)-1-methyl-1H-1,2,4-triazol-5-yl)-4-fluorophenoxy)-6-fluoro-4-vinyl-1H-indole (Step A product of Example 93A, 93B) with 5-(3-(4-(2-(3-bromophenyl)-5-((2-methylbut-3-yn-2-yl)oxy)pentan-2-yl)-1-methyl-1H-imidazol-2-yl)phenoxy)-6-fluoro-4-vinyl-1H-indole (Step A product of this example, 1.0 g, 1.56 mmol), the reaction procedure sequence (Steps B. C, D and E) described for Example 93A, 93B for making the corresponding intermediate was used to prepare the title compound (220 mg). MS (ESI): 669, 671 m/z [M+H]⁺, retention time: 1.82 minutes, purity: 60% (254 nm) (LC-MS Method 27).

6-(3-Bromophenyl)-24-fluoro-3,6,11,11-tetramethyl-10,26-dioxa-3,13,14,15,21,33-hexazahexacyclo[25.3.1.12,5.112,15.017,25.018,22]tritriaconta-1(31),2(33),4,12(32),13,17,19,22,24,27,29-undecaene

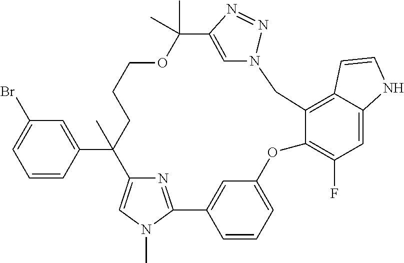 [SMILES: Cn1cc2nc1-c1cccc(c1)Oc1c(F)cc3[nH]ccc3c1Cn1cc(nn1)C(C)(C)OCCCC2(C)c1cccc(Br)c1]

Step C: To a stirred solution of Step B product (220 mg, 0.32 mmol) in dichloromethane (100 mL) was added tris((1-benzyl-1H-1,2,3-triazol-4-yl)methyl)amine (TABA, 20 mg, 0.033 mmol) and tetrakis(acetonitrile)copper(I) tetrafluoroborate (10 mg, 0.033 mmol). The mixture was heated to 45° C. and stirred for 70 hours and concentrated. The residue was purified by flash chromatography to afford the title compound (140 mg, 60%). MS (ESI): 669, 671 m/z [M+H]⁺, retention time: 1.69 minutes, purity: 90% (254 nm) (LC-MS Method 27).

Ethyl (E)-3-[3-(24-fluoro-3,6,11,11-tetramethyl-10,26-dioxa-3,13,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]-2-methyl-prop-2-enoate

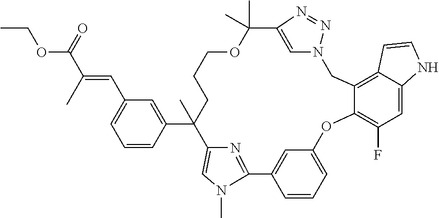 [SMILES: CCOC(=O)/C(C)=C/c1cccc(C2(C)CCCOC(C)(C)c3cn(nn3)Cc3c(c(F)cc4[nH]ccc34)Oc3cccc(c3)-c3nc2cn3C)c1]

Step D: To a stirred and degassed solution of Step C product (130 mg, 0.19 mmol) in 1,4-dioxane (20 mL) and water (5 mL) was added [1,1′-bis(diphenylphosphino)ferrocene]dichloropalladium (II) (Pd(dppf)Cl₂, 14.2 mg, 0.019 mmol), ethyl (E)-2-methyl-3-(4,4,5,5-tetramethyl-1,3,2-dioxaborolan-2-yl)prop-2-enoate (93.2 mg, 0.38 mmol) and cesium carbonate (127 mg, 0.38 mmol). The mixture was stirred at 100° C. for 16 hours, cooled to room temperature, and quenched with 50 mL of water. The solution was extracted with ethyl acetate (3×20 mL). The combined organic phases were washed with water (20 mL), dried over sodium sulfate, and concentrated. The crude material was purified by flash chromatography to afford the title compound (100 mg, 75%). MS (ESI): 703 m/z [M+H]⁺, retention time: 1.74 minutes, purity: 90% (254 nm) (LC-MS Method 27).

Ethyl 3-[3-(24-fluoro-3,6,11,11-tetramethyl-10,26-dioxa-3,13,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]-2-methyl-propanoate

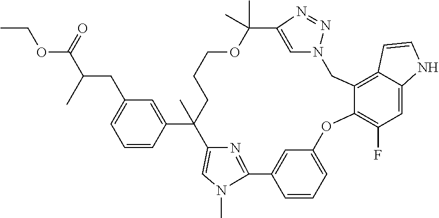 [SMILES: CCOC(=O)C(C)Cc1cccc(C2(C)CCCOC(C)(C)c3cn(nn3)Cc3c(c(F)cc4[nH]ccc34)Oc3cccc(c3)-c3nc2cn3C)c1]

Step E: To a stirred solution of Step D product (100 mg, 0.14 mmol) in ethanol (10 mL) was added palladium on active carbon (10%, 50% wet, 30 mg). The mixture was stirred at 45° C. for 16 hours, then filtered through a pad of Celite. The filtrate was concentrated to give the crude title compound (90 mg, 90%). MS (ESI): 705 m/z [M+H]⁺, retention time: 1.73 minutes, purity: 90% (254 nm) (LC-MS Method 27).

Compound 106.3-[3-(24-fluoro-3,6,11,11-tetramethyl-10,26-dioxa-3,13,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]-2-methyl-propanoic acid

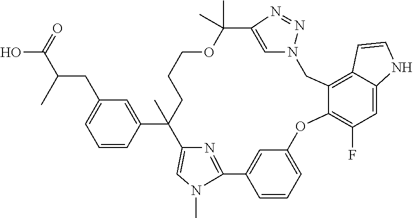 [SMILES: CC(Cc1cccc(C2(C)CCCOC(C)(C)c3cn(nn3)Cc3c(c(F)cc4[nH]ccc34)Oc3cccc(c3)-c3nc2cn3C)c1)C(=O)O]

Step F: A mixture of Step E product (0.13 mmol, 90 mg) and lithium hydroxide (0.2 M in methanol: water: tetrahydrofuran=1:1:3, 2.5 mL) was stirred at 45° C. to for two hours, then cooled to room temperature and acidified with 1 N hydrochloric acid (1 M in water, 5 mL). The solution was extracted with ethyl acetate (2×15 mL), the combined organic phase was washed with brine, dried over sodium sulfate, and concentrated. The residue was purified by chromatography to afford the title compound (15 mg, 18%) as a white solid. MS (ESI): 677 m/z [M+H]⁺, retention time: 1.46 minutes, purity: 60% (214 nm) (LC-MS Method 27). ¹H NMR (400 MHz, CD₃OD) δ67.56-7.45 (m, 2H), 7.42-7.36 (m, 1H), 7.34-7.23 (m, 3H), 7.16-6.95 (m, 5H), 6.71 (s, 1H), 6.28 (s, 1H), 5.94 (d, J=15.2 Hz, 1H), 5.83 (d, J=14.8 Hz, 1H), 3.68 (s, 3H), 3.10-3.06 (s, 1H), 2.91-2.84 (m, 1H), 2.70-2.55 (s, 1H), 2.20-2.09 (m, 1H), 2.03-1.91 (m, 1H), 1.56-1.42 (m, 7H), 1.41-1.25 (m, 7H), 1.20-1.05 (m, 2H) ppm.

Example 107. Compound 107 and Compound 108. Regio-isomers 1 of 2-methyl-3-[3-(9,9,24,30-tetrafluoro-3,6-dimethyl-11,26-dioxa-3,4,15,21,32,33-hexazahexacyclo-[25.3.1.12,5.112,15.017,25.018,22]tritriaconta-1(31),2(33),4,12(32),13,17,19,22,24,27,29-undecaen-6-yl)phenyl]propanoic acid

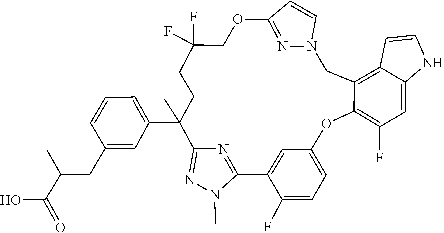 [SMILES: CC(Cc1cccc(C2(C)CCC(F)(F)COc3ccn(n3)Cc3c(c(F)cc4[nH]ccc34)Oc3ccc(F)c(c3)-c3nc2nn3C)c1)C(=O)O]

and

Example 108. Compound 108. Regio-isomer 2 of 2-methyl-3-[3-(9,9,25,31-tetrafluoro-3,6-dimethyl-11,27-dioxa-3,4,15,16,22,33-hexazahexacyclo[26.3.1.12,5.012,16.018,26.019,23]tritriaconta-1(32),2(33),4,12,14,18,20,23,25,28,30-undecaen-6-yl)phenyl]propanoic acid

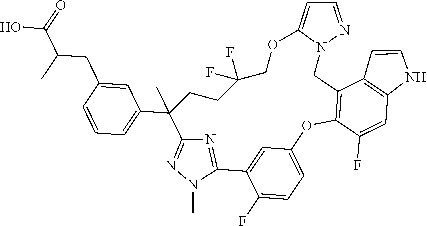 [SMILES: CC(Cc1cccc(C2(C)CCC(F)(F)COc3ccnn3Cc3c(c(F)cc4[nH]ccc34)Oc3ccc(F)c(c3)-c3nc2nn3C)c1)C(=O)O]

Exchanging ethyl 3-(3-(6-((1H-pyrazol-3-yl)oxy)-2,5,5-trimethyl-1-(2-methylhydrazineyl)-1-oxohexan-2-yl)phenyl)-2-methylpropanoate (Intermediate 83-5) with ethyl 3-(3-(6-((1H-pyrazol-3-yl)oxy)-5,5-difluoro-2-methyl-1-(2-methylhydrazineyl)-1-oxohexan-2-yl)phenyl)-2-methyl-propanoate (Intermediate 97, 320 mg, 0.686 mmol), the reaction procedure sequence (Steps A, B, C and E) described for Example 99A & 99B, 99A & 99B was used to prepare the title compounds. The two regio-isomers formed in corresponding Step C were separated by automated silica gel chromatography. The first eluent, Regioisomer 1 (96 mg), was further deprotected and hydrolyzed to Compound 107 (58 mg, 75%); The second eluent, Regioisomer 2 (46 mg), was further deprotected and hydrolyzed to Compound 108 (25 mg, 67%), as described in Step E of Example 99A & 99B, 99A & 99B.

Compound 107: MS (ESI): 703 m/z [M+H]⁺, retention time: 2.14 minutes, purity: >99% (214 nm) (LC-MS Method 5). ¹H NMR (400 MHz, CD₃OD) δ 7.40-7.30 (m, 3H), 7.28-7.23 (m, 2H), 7.19-7.15 (m, 1H), 7.05-7.01 (m, 3H), 6.64 (d, J=2.8 Hz, 1H), 6.33-6.30 (m, 1H), 5.49-5.37 (m, 3H), 4.18-4.07 (m, 1H), 3.91-3.83 (m, 1H), 3.71 (s, 3H), 2.98-2.92 (m, 1H), 2.65-2.55 (m, 2H), 2.32-2.17 (m, 2H), 2.10-2.00 (m, 1H), 1.83-1.72 (m, 1H), 1.63 (s, 3H), 1.08-1.05 (m, 3H) ppm.

Compound 108: MS (ESI): 703 m/z [M+H]⁺, retention time: 2.08 minutes, purity: >99% (254 nm) (LC-MS Method 5). ¹H NMR (400 MHz, CD₃OD) δ 7.55-7.51 (m, 1H), 7.43-7.34 (m, 2H), 7.21-7.12 (m, 3H), 7.01 (d, J=7.8 Hz, 1H), 6.97-6.89 (m, 2H), 6.73 (dd, J=5.6, 3.2 Hz, 1H), 5.86 (d, J=2.0 Hz, 1H), 5.59 (s, 2H), 5.15 (d, J=3.2 Hz, 1H), 4.20-4.13 (m, 2H), 3.85 (d, J=2.4 Hz, 3H), 2.94-2.87 (m, 1H), 2.59-2.52 (m, 3H), 2.42-2.35 (m, 1H), 2.10-2.02 (m, 1H), 1.83-1.75 (m, 1H), 1.71 (s, 3H), 1.06-1.02 (m, 3H) ppm.

Example 109. Compound 109A, 109B. 3-[3-(24,30-difluoro-11,11-dimethyl-9,26-dioxa-5,13,14,15,21,33-hexazahexacyclo[25.3.1.12,5.112,15.017,25.018,22]tritriaconta-1(31),2(33),3,12(32),13,17,19,22,24,27,29-undecaen-6-yl)phenyl]-2-methyl-propanoic acid

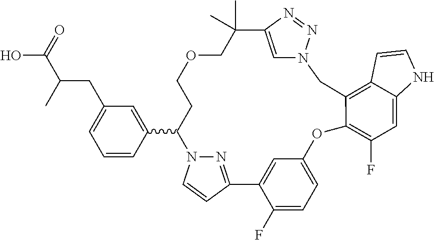 [SMILES: CC(Cc1cccc(C2CCOCC(C)(C)c3cn(nn3)Cc3c(c(F)cc4[nH]ccc34)Oc3ccc(F)c(c3)-c3ccn2n3)c1)C(=O)O]

and

Example 110. Compound 110. 24,30-difluoro-11,11-dimethyl-6-phenyl-9,26-dioxa-5,13,14,15,21,33-hexazahexacyclo[25.3.1.12,5.112,15.017,25.018,22]tritriaconta-1(31),2(33),3,12(32),13,17,19,22,24,27,29-undecaene

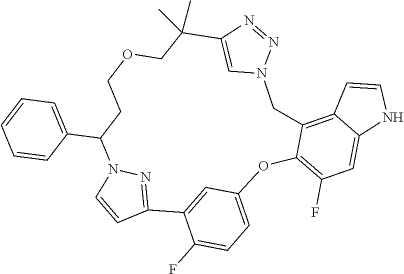 [SMILES: CC1(C)COCCC(c2ccccc2)n2ccc(n2)-c2cc(ccc2F)Oc2c(F)cc3[nH]ccc3c2Cn2cc1nn2]

(5-(3-(1-(1-(3-Bromophenyl)-3-((2,2-dimethylbut-3-yn-1-yl)oxy)propyl)-1H-pyrazol-3-yl)-4-fluorophenoxy)-6-fluoro-1-tosyl-1H-indol-4-yl)methanol

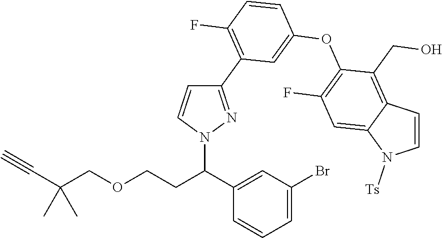 [SMILES: C#CC(C)(C)COCCC(c1cccc(Br)c1)n1ccc(-c2cc(Oc3c(F)cc4c(ccn4S(=O)(=O)c4ccc(C)cc4)c3CO)ccc2F)n1]

Step A: To a stirred and degassed solution of (6-fluoro-5-(4-fluoro-3-(1H-pyrazol-3-yl)phenoxy)-1-tosyl-1H-indol-4-yl)methanol (Intermediate 55-3, 1 g, 2.02 mmol) and 1-bromo-3-(1-bromo-3-((2,2-dimethylbut-3-yn-1-yl)oxy)propyl)benzene (Intermediate 98, 755 mg, 2.02 mmol) in N,N-dimethylformamide (30 mL) was added cesium carbonate (1.31 g, 4.04 mmol). The mixture was stirred at room temperature overnight, then quenched with water (150 mL), and extracted with ethyl acetate (3×50 mL). The combined organic extracts were washed with brine (3×50 m), dried over sodium sulfate, and concentrated. The obtained crude title compound (1.94 g, crude) was used in the next step without further purification. MS (ESI): 788, 790 m/z [M+H]⁺, retention time: 2.26 minutes, purity: 67% (254 nm) (LC-MS Method 2).

(5-(3-(1-(1-(3-Bromophenyl)-3-((2,2-dimethylbut-3-yn-1-yl)oxy)propyl)-1H-pyrazol-3-yl)-4-fluorophenoxy)-6-fluoro-1H-indol-4-yl)methanol

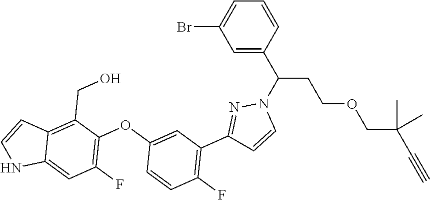 [SMILES: C#CC(C)(C)COCCC(c1cccc(Br)c1)n1ccc(-c2cc(Oc3c(F)cc4[nH]ccc4c3CO)ccc2F)n1]

Step B: To a stirred solution of crude Step A product (1.94 g, crude) in tetrahydrofuran (30 mL) and methanol (10 mL) was added lithium hydroxide (1M in tetrahydrofuran, 10 mL). The mixture was stirred at room temperature for two hours, then diluted with water (50 mL), and extracted with ethyl acetate (3×30 mL). The combined organic extracts were washed with brine, dried over sodium sulfate, and concentrated. The residue was purified by automated flash chromatography (40 g silica gel column, eluting with 0-60% ethyl acetate in petroleum ether) to give the title compound (905 mg, 70% two steps) as a solid. MS (ESI): 634, 636 m/z [M+H]⁺, retention time: 1.53 minutes, purity: 80% (254 nm) (LC-MS Method 24).

4-(Azidomethyl)-5-(3-(1-(1-(3-bromophenyl)-3-((2,2-dimethylbut-3-yn-1-yl)oxy)propyl)-1H-pyrazol-3-yl)-4-fluorophenoxy)-6-fluoro-1H-indole

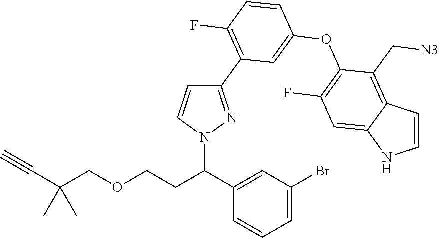 [SMILES: C#CC(C)(C)COCCC(c1cccc(Br)c1)n1ccc(-c2cc(Oc3c(F)cc4[nH]ccc4c3CN=[N+]=[N-])ccc2F)n1]

Step C: To a stirred and degassed solution of Step B product (905 mg, 1.43 mmol) in tetrahydrofuran (30 mL) was added diphenylphosphoryl azide (1.18 g, 4.29 mmol) and 1,8-diazabicyclo[5.4.0]undec-7-ene (435 mg, 2.86 mmol). The mixture was refluxed for 2 days, then diluted with water (50 mL), and extracted with ethyl acetate (3×30 mL). The combined organic extracts were washed with brine (3×30 mL), dried over sodium sulfate, and concentrated. The residue was purified by flash chromatography (40 g silica gel column, eluting with 0-60% ethyl acetate in petroleum ether) to give the title compound (655 mg, 70%) as solid. MS (ESI): 659, 661 m/z [M+H]⁺, retention time: 2.24 minutes, purity: 80% (214 nm) (LC-MS Method 24).

Compounds 109A, 109B: Diastereomers 1 and 2 of 3-[3-(24,30-difluoro-11,11-dimethyl-9,26-dioxa-5,13,14,15,21,33-hexazahexacyclo-[25.3.1.12,5.112,15.017,25.018,22]tritriaconta-1(31),2(33),3,12(32),13,17,19,22,24,27,29-undecaen-6-yl)phenyl]-2-methyl-propanoic acid

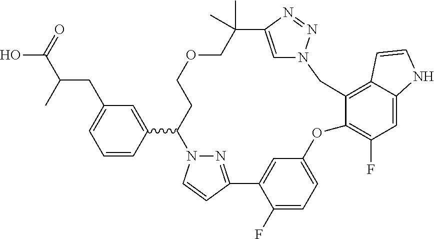 [SMILES: CC(Cc1cccc(C2CCOCC(C)(C)c3cn(nn3)Cc3c(c(F)cc4[nH]ccc34)Oc3ccc(F)c(c3)-c3ccn2n3)c1)C(=O)O]

and

Compound 110: 24,30-difluoro-11,11-dimethyl-6-phenyl-9,26-dioxa-5,13,14,15,21,33-hexazahexacyclo[25.3.1.12,5.112,15.017,25.018,22]tritriaconta-1(31),2(33),3,12(32),13,17,19,22,24,27,29-undecaene

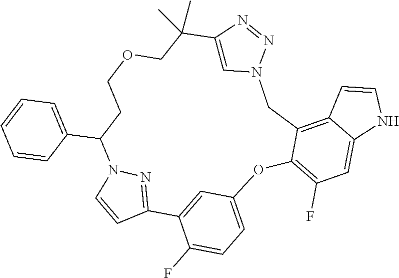 [SMILES: CC1(C)COCCC(c2ccccc2)n2ccc(n2)-c2cc(ccc2F)Oc2c(F)cc3[nH]ccc3c2Cn2cc1nn2]

Step D: Exchanging 4-(azidomethyl)-5-(3-(4-(2-(3-bromophenyl)-5-((2-methylbut-3-yn-2-yl)oxy)pentan-2-yl)-1-methyl-1H-imidazol-2-yl)phenoxy)-6-fluoro-1H-indole (Step B product of Example 106) with 4-(azidomethyl)-5-(3-(1-(1-(3-bromophenyl)-3-((2,2-dimethylbut-3-yn-1-yl)oxy)propyl)-1H-pyrazol-3-yl)-4-fluorophenoxy)-6-fluoro-1H-indole (Step C product of this Example, 655 mg, 1 mmol), the reaction procedure sequence (Steps C to E) described for Example 106 was used to prepare the title compounds. The racemic esters, along with inseparable debromination product (250 mg) from the Suzuki coupling, was subjected to chiral SFC separation using SFC-80 (Thar, Waters) under the following: Column: IG 20*250 mm, 10 μm; Column temperature: 35° C.; Mobile phase: carbon dioxide/isopropanol (0.2% methanol ammonia)=65/35; Flow rate: 80 g/minute; Back pressure: 100 bar; Detection wavelength: 214 nm; Cycle time: 4.1 minutes; Sample solution: 450 mg dissolved in 30 mL of methanol; Injection volume: 1.9 mL. The first eluent is the mixture of enantiomer 1 and de-brominated product (90 mg). This mixture was separated after hydrolysis, following conditions described in corresponding Step E, to Compound 109A (55.4 mg) and Compound 110 (4.1 mg). The second eluent, Enantiomer 2 (90 mg), was further hydrolyzed to Compound 109B (70.6 mg).

Compound 109A: MS (ESI): 667 m/z [M+H]⁺, retention time: 1.68 minutes, purity: 99% (214 nm) (LC-MS Method 8). ¹H NMR (400 MHz, CD₃OD) δ 7.56 (d, J=2.4 Hz, 1H), 7.39-7.37 (m, 1H), 7.31-7.29 (m, 3H), 7.19-7.15 (m, 3H), 7.08-7.06 (m, 2H), 7.03-6.98 (m, 1H), 6.65-6.63 (m, 1H), 6.28 (t, J=2.8 Hz, 1H), 5.91-5.84 (m, 2H), 5.36-5.30 (m, 1H), 3.30-3.20 (m, 3H), 3.20-3.13 (m, 1H), 2.97-2.90 (m, 1H), 2.67-2.48 (m, 3H), 2.12-2.01 (m, 1H), 1.16 (s, 3H), 1.05 (d, J=6.4 Hz, 3H), 1.02 (s, 3H) ppm.

Compound 110: MS (ESI): 581 m/z [M+H]⁺, retention time: 2.11 minutes, purity: 97% (214 nm) (LC-MS Method 8). ¹H NMR (400 MHz, CD₃OD) δ 7.58 (d, J=2.4 Hz, 1H), 7.38-7.06 (m, 11H), 6.65 (dd, J=4.4, 2.4 Hz, 1H), 6.30 (dd, J=3.2, 0.8 Hz, 1H), 5.92-5.85 (m, 2H), 5.38 (dd, J=12.0, 2.8 Hz, 1H), 3.30-3.23 (m, 3H), 3.17 (d, J=9.2 Hz, 1H), 2.58-2.49 (m, 1H), 2.12-2.04 (m, 1H), 1.16 (s, 3H), 1.01 (s, 3H) ppm.

Compound 109B: MS (ESI): 667 m/z [M+H]⁺, retention time: 1.68 minutes, purity: 99% (214 nm) (LC-MS Method 8). ¹H NMR (400 MHz, CD₃OD) δ 7.56 (d, J=2.0 Hz, 1H), 7.38 (dd, J=10.8, 6.8 Hz, 1H), 7.31-7.29 (m, 3H), 7.19-7.15 (m, 3H), 7.07 (d, J=5.6 Hz, 2H), 7.03-6.98 (m, 1H), 6.65-6.62 (m, 1H), 6.28 (t, J=2.8 Hz, 1H), 5.91-5.82 (m, 2H), 5.36-5.31 (m, 1H), 3.29-3.10 (m, 4H), 2.97-2.90 (m, 1H), 2.66-2.48 (m, 3H), 2.12-2.01 (m, 1H), 1.16 (s, 3H), 1.05 (d, J=6.4 Hz, 3H), 1.02 (s, 3H) ppm.

Example 111. Compound 111A and Compound 111B. Diastereomers 1 and 2 of 3-[2-fluoro-3-(24-fluoro-11,11-dimethyl-9,26-dioxa-5,13,14,15,21,33-hexazahexacyclo-[25.3.1.12,5.112,15.017,25.018,22]tritriaconta-1(31),2(33),3,12(32),13,17,19,22,24,27,29-undecaen-6-yl)phenyl]-2-methyl-propanoic acid

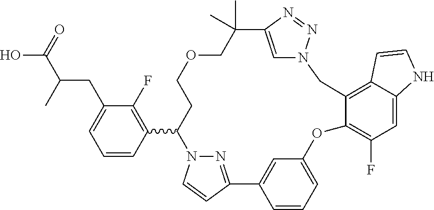 [SMILES: CC(Cc1cccc(C2CCOCC(C)(C)c3cn(nn3)Cc3c(c(F)cc4[nH]ccc34)Oc3cccc(c3)-c3ccn2n3)c1F)C(=O)O]

and

Example 112. Compound 112A and Compound 112B. Enantiomers 1 and 2 of 24-fluoro-6-(2-fluorophenyl)-11,11-dimethyl-9,26-dioxa-5,13,14,15,21,33-hexazahexacyclo-[25.3.1.12,5.112,15.017,25.018,22]tritriaconta-1(31),2(33),3,12(32),13,17,19,22,24,27,29-undecaene

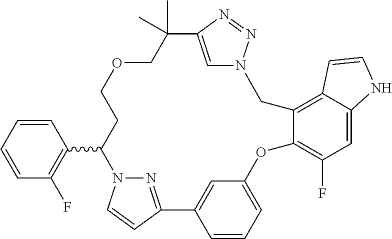 [SMILES: CC1(C)COCCC(c2ccccc2F)n2ccc(n2)-c2cccc(c2)Oc2c(F)cc3[nH]ccc3c2Cn2cc1nn2]

Exchanging 1-bromo-3-(1-bromo-3-((2,2-dimethylbut-3-yn-1-yl)oxy)propyl)benzene (Intermediate 98) with 1-bromo-3-(1-bromo-3-((2,2-dimethylbut-3-yn-1-yl)oxy)propyl)-2-fluorobenzene (Intermediate 98-1, 1.04 g, 2.66 mmol), the reaction procedure sequence (Steps A to D) described for Example 109A, 109B was used to prepare the title compounds. The mixture of racemic ethyl ester and de-brominated product was subject to chiral SFC separation using SFC-80 (Thar, Waters) under the following condition: Column: OZ 20*250 mm, 10 μm; Column temperature: 35° C.; Mobile phase: carbon dioxide/methanol (0.2% methanol ammonia)=45/55; Flow rate: 80 g/minute; Back pressure: 100 bar; Detection wavelength: 214 nm; Cycle time: 4.2 minutes; Sample solution: 250 mg dissolved in 30 mL of methanol. Injection volume: 1.0 mL. The first eluent is Enantiomer 1 of de-brominated product, designated as Compound 112A (7.3 mg); The second eluent (70 mg) is a mixture of Enantiomer 2 of debrominated product and Diastereomer 1 of the ethyl ester, which were separated after hydrolysis, to give Compound 112B (3.0 mg) and Compound 111A (20.9 mg); The third eluent (60 mg), Diastereomer 2 of the ethyl ester, was further hydrolyzed to Compound 111B (49.4 mg, 86%), following the conditions described in Step C of Example 109A, 109B.

Compound 112A: MS (ESI): 581 m/z [M+H]⁺, retention time: 2.00 minutes, purity: 98% (214 nm) (LC-MS Method 2). ¹H NMR (400 MHz, CD₃OD) δ 7.63 (d, J=2.4 Hz, 1H), 7.45-7.36 (m, 3H), 7.31 (d, J=3.2 Hz, 1H), 7.29-7.21 (m, 4H), 7.15-7.05 (m, 3H), 6.63 (d, J=2.4 Hz, 1H), 6.33 (dd, J=3.2, 0.8 Hz, 1H), 5.92-5.82 (m, 2H), 5.71 (dd, J=11.6, 2.4 Hz, 1H), 3.42-3.35 (m, 2H), 3.28 (d, J=9.2 Hz, 1H), 3.14 (d, J=9.2 Hz, 1H), 2.70-2.61 (m, 1H), 2.10-2.02 (m, 1H), 1.19 (s, 3H), 1.00 (s, 3H) ppm.

Compound 112B: MS (ESI): 581 m/z [M+H]⁺, retention time: 2.00 minutes, purity: >99% (214 nm) (LC-MS Method 2). ¹H NMR (400 MHz, CD₃OD) δ 7.63 (d, J=2.0 Hz, 1H), 7.45-3.37 (m, 3H), 7.31 (d, J=3.2 Hz, 1H), 7.29-7.21 (m, 4H), 7.15-7.05 (m, 3H), 6.63 (d, J=2.4 Hz, 1H), 6.33 (dd, J=3.2, 0.8 Hz, 1H), 5.92-5.82 (m, 2H), 5.71 (dd, J=12.0, 2.4 Hz, 1H), 3.42-3.36 (m, 2H), 3.28 (d, J=9.2 Hz, 1H), 3.14 (d, J=9.2 Hz, 1H), 2.71-2.61 (m, 1H), 2.09-2.02 (m, 1H), 1.19 (s, 3H), 1.00 (s, 3H) ppm.

Compound 111A: MS (ESI): 667 m/z [M+H]⁺, retention time: 1.92 minutes, purity: >99% (214 nm) (LC-MS Method 2). ¹H NMR (400 MHz, CD₃OD) δ 7.63 (d, J=2.4 Hz, 1H), 7.45-7.36 (m, 3H), 7.31 (d, J=3.2 Hz, 1H), 7.25-7.21 (m, 3H), 7.17-7.13 (m, 1H), 7.10-6.94 (m, 2H), 6.63 (d, J=2.4 Hz, 1H), 6.34-6.32 (m, 1H), 5.92-5.82 (m, 2H), 5.71 (d, J=11.6 Hz, 1H), 3.41-3.37 (m, 2H), 3.28 (d, J=9.2 Hz, 1H), 3.14 (d, J=9.2 Hz, 1H), 3.03-2.95 (m, 1H), 2.76-2.58 (m, 3H), 2.10-2.01 (m, 1H), 1.19 (s, 3H), 1.14-1.12 (m, 3H), 1.01 (s, 3H) ppm. Chiral HPLC: 100% (Method condition: Chiral SFC: Column: OX-H; Mobile: carbon dioxide (75%)/35% methanol (0.2% methanol ammonia as additive); Flow rate: 4 mL/minute; Column Temperature: 40° C.; Back Pressure: 120 bar)

Compound 111B: MS (ESI): 667 m/z [M+H]⁺, retention time: 1.92 minutes, purity: >99% (214 nm) (LC-MS Method 2). ¹H NMR (400 MHz, CD₃OD) δ 7.63 (d, J=2.4 Hz, 1H), 7.45-7.36 (m, 3H), 7.31 (d, J=3.2 Hz, 1H), 7.26-7.22 (m, 3H), 7.17-7.14 (m, 1H), 7.02-6.96 (m, 2H), 6.63 (d, J=2.4 Hz, 1H), 6.34-6.32 (m, 1H), 5.92-5.82 (m, 2H), 5.71 (d, J=9.2 Hz, 1H), 3.41-3.37 (m, 2H), 3.28 (d, J=8.8 Hz, 1H), 3.14 (d, J=8.8 Hz, 1H), 3.02-2.96 (m, 1H), 2.78-2.60 (m, 3H), 2.10-2.03 (m, 1H), 1.19 (s, 3H), 1.14-1.12 (m, 3H), 1.01 (s, 3H) ppm. Chiral HPLC: 100%

Example 113. Compound 113A and Compound 113B. Diastereomers 1 and 2 of 2-methyl-3-[3-(11,11,24,30-tetrafluoro-6-methyl-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-6-yl)phenyl]propanoic acid

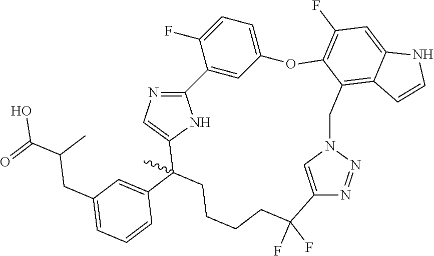 [SMILES: CC(Cc1cccc(C2(C)CCCCC(F)(F)c3cn(nn3)Cc3c(c(F)cc4[nH]ccc34)Oc3ccc(F)c(c3)-c3ncc2[nH]3)c1)C(=O)O]

4-(Azidomethyl)-5-(3-(5-(2-(3-bromophenyl)-7,7-difluoronon-8-yn-2-yl)-1H-imidazol-2-yl)-4-fluorophenoxy)-6-fluoro-1H-indole

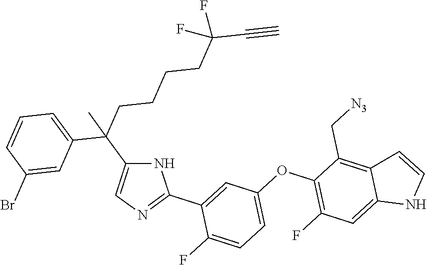 [SMILES: C#CC(F)(F)CCCCC(C)(c1cccc(Br)c1)c1cnc(-c2cc(Oc3c(F)cc4[nH]ccc4c3CN=[N+]=[N-])ccc2F)[nH]1]

Step A: To a stirred solution of 5-((4-(azidomethyl)-6-fluoro-1H-indol-5-yl)oxy)-2-fluorobenzimidamide (Intermediate 6-1, 1.48 g, 4.35 mmol) in N,N-dimethylformamide (40 mL) was added 1-bromo-3-(3-bromophenyl)-8,8-difluoro-3-methyldec-9-yn-2-one (Intermediate 2-49, 1.9 g, 4.35 mmol) and sodium bicarbonate (732 mg, 8.71 mmol). The reaction mixture was heated at 80° C. overnight, cooled to room temperature, and diluted with ethyl acetate (100 mL). The mixture was washed with water, brine, dried over sodium sulfate, filtered and concentrated. The residue was purified by automated flash chromatography (40 g silica gel column, eluting with 0-40% ethyl acetate in petroleum ether) to give the title compound (1.5 g, 51%) as a yellow solid. MS (ESI): 679, 681 m/z [M+H]⁺, retention time: 1.90 minutes, purity: 85% (254 nm) (LC-MS Method 5).

Compounds 113A and 113B: Diastereomers 1 and 2 of 2-methyl-3-[3-(11,11,24,30-tetrafluoro-6-methyl-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-6-yl)phenyl]propanoic acid

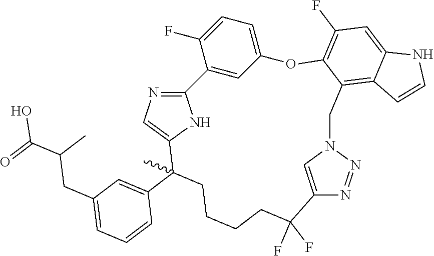 [SMILES: CC(Cc1cccc(C2(C)CCCCC(F)(F)c3cn(nn3)Cc3c(c(F)cc4[nH]ccc34)Oc3ccc(F)c(c3)-c3ncc2[nH]3)c1)C(=O)O]

Exchanging 4-(azidomethyl)-5-(3-(4-(2-(3-bromophenyl)-5-((2-methylbut-3-yn-2-yl)oxy)pentan-2-yl)-1-methyl-1H-imidazol-2-yl)phenoxy)-6-fluoro-1H-indole (Step B product of Example 106) with 4-(azidomethyl)-5-(3-(5-(2-(3-bromophenyl)-7,7-difluoronon-8-yn-2-yl)-1H-imidazol-2-yl)-4-fluorophenoxy)-6-fluoro-1H-indole (Step A product of this example, 1.5 g, 2.2 mmol), The reaction procedure sequence (Steps C to F) described for Example 106 was used to prepare the title compounds. The racemic ethyl ester (350 mg), obtained from corresponding Step E, was subject to chiral SFC separation using SFC-80 (Thar, Waters) under the following conditions: Column: OJ 20*250 mm, 10 μm; Column temperature: 35° C.; Mobile phase: carbon dioxide/ethanol (0.5% methanol ammonia)=70/30; Flow rate: 80 g/minute; Back pressure: 100 bar; Detection wavelength: 214 nm; Cycle time: 4.8 minutes; Sample solution: 350 mg dissolved in 20 ml methanol; Injection volume: 0.6 mL. The first eluent, Diastereomer 1 (120 mg, 34%), was further hydrolyzed to Compound 113A (80 mg, 70%); The second eluent, Diastereomer 2, (140 mg, 40%), was further hydrolyzed to Compound 113B (74 mg, 55%).

Compound 113A: MS (ESI): 687 m/z [M+H]⁺, retention time: 1.56 minutes, purity: >99% (254 nm) (LC-MS Method 5). ¹H NMR (400 MHz, CD₃OD) δ 7.84 (s, 1H), 7.40 (d, J=3.2 Hz, 1H), 7.33 (dd, J=10.4, 0.8 Hz, 1H), 7.27-7.24 (m, 2H), 7.12 (t, J=7.6 Hz, 1H), 7.05-6.96 (m, 5H), 6.70 (d, J=3.2 HZ, 1H), 6.01 (d, J=14.4 Hz, 1H), 5.88 (d, 14.4 Hz, 1H), 2.97-2.89 (m, 1H), 2.63-2.53 (m, 2H), 2.11-1.99 (m, 3H), 1.93-1.79 (m, 1H), 1.52-1.43 (m, 4H), 1.37-1.28 (m, 1H), 1.08-1.06 (m, 3H), 1.02-0.89 (m, 2H) ppm.

Compound 113B: MS (ESI): 687 m/z [M+H]⁺, retention time: 1.56 minutes, purity: >99% (254 nm) (LC-MS Method 5). ¹H NMR (400 MHz, CD₃OD) δ 7.84 (s, 1H), 7.40 (d, J=3.2 Hz, 1H), 7.33 (dd, J=10.4, 0.8 Hz, 1H), 7.27-7.24 (m, 2H), 7.12 (t, J=7.6 Hz, 1H), 7.05-6.96 (m, 5H), 6.70 (d, J=3.2 Hz, 1H), 6.02 (d, J=14.4 Hz, 1H), 5.88 (d, J=14.4 Hz, 1H), 2.97-2.90 (m, 1H), 2.63-2.53 (m, 2H), 2.10-1.99 (m, 3H), 1.93-1.79 (m, 1H), 1.52-1.43 (m, 4H), 1.37-1.28 (m, 1H), 1.008-1.06 (m, 3H), 1.02-0.89 (m, 2H) ppm.

Example 114. Compound 114A and Compound 114B. Diastereomers 1 and 2 of 3-[3-(24,30-difluoro-6,11,11-trimethyl-26-oxa-3,13,14,15,21,33-hexazahexacyclo-[25.3.1.12,5.112,15.017,25.018,22]tritriaconta-1(31),2,4,12(32),13,17,19,22,24,27,29-undecaen-6-yl)phenyl]-2-methyl-propanoic acid

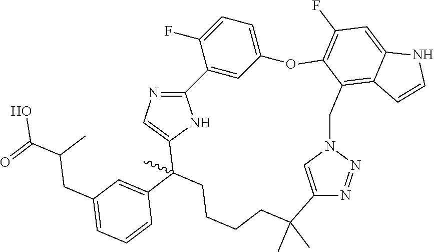 [SMILES: CC(Cc1cccc(C2(C)CCCCC(C)(C)c3cn(nn3)Cc3c(c(F)cc4[nH]ccc34)Oc3ccc(F)c(c3)-c3ncc2[nH]3)c1)C(=O)O]

Exchanging 1-bromo-3-(3-bromophenyl)-8,8-difluoro-3-methyldec-9-yn-2-one (Intermediate 2-49) with 1-bromo-3-(3-bromophenyl)-3,8,8-trimethyldec-9-yn-2-one (Intermediate 2-39, 2 g, 4.72 mmol), the reaction procedure sequence (Steps A and B) described for Example 113A and 113B was used to prepare the title compounds. The racemic ethyl ester (700 mg), obtained from corresponding Step B, was subject to chiral SFC separation using SFC-80 (Thar, Waters) under the following condition: Column: OJ 20*250 mm, 10 μm; Column temperature: 35° C.; Mobile phase: carbon dioxide/methanol (0.5% methanol ammonia)=75/25; Flow rate: 80 g/minute; Back pressure: 100 bar; Detection wavelength: 214 nm; Cycle time: 5.6 minutes; Sample solution: 700 mg dissolved in 25 ml methanol; Injection volume: 1.0 mL. The first eluent of the ethyl ester was designated as Diastereomer 1 (300 mg, 43%). The second eluent was designated as Diastereomer 2 (290 mg, 41%). 60 mg of the first eluent was further hydrolyzed to Compound 114A (35 mg, 61%); 60 mg of the second eluent was further hydrolyzed to Compound 114B (35.5 mg, 61%).

Compound 114A: MS (ESI): 679 m/z [M+H]⁺, retention time: 1.54 minutes, purity: 97% (214 nm) (LC-MS Method 26). ¹H NMR (400 MHz, CD₃OD) δ 7.33 (d, J=3.2 Hz, 1H), 7.31 (s, 1H), 7.27-7.24 (m, 3H), 7.09-7.01 (m, 4H), 6.97-6.91 (m, 2H), 6.60 (d, J=3.2 Hz, 1H), 5.92 (d, J=14.4 Hz, 1H), 5.74 (d, J=14.4 Hz, 1H), 2.94-2.82 (m, 1H), 2.61-2.46 (m, 2H), 1.99 (t, J=7.6 Hz, 2H), 1.48-1.36 (m, 5H), 1.25-1.19 (m, 1H), 1.11 (s, 3H), 1.05-0.89 (m, 7H), 0.81-0.59 (m, 2H) ppm. Chiral purity: 100% (Chiral SFC Column: OJ-H; Mobile: 15% methanol (012% methanol ammonia); Flow Rate: 4 mL/minute; Temperature: 40° C.; Back Pressure: 120 bar).

Compound 114B: MS (ESI): 679 m/z [M+H]⁺, retention time: 1.54 minutes, purity: 99% (214 nm) (LC-MS Method 26). ¹H NMR (400 MHz, CD₃OD) δ 7.32 (d, J=3.2 Hz, 1H), 7.31 (s, 1H), 7.27-7.22 (m, 3H), 7.10-7.01 (m, 4H), 6.97-6.90 (m, 2H), 6.59 (d, J=2.8 Hz, 1H), 5.92 (d, J=14.4 Hz, 1H), 5.74 (d, J=14.4 Hz, 1H), 2.92-2.87 (m, 1H), 2.59-2.51 (m, 2H), 1.99 (t, J=7.6 Hz, 2H), 1.50-1.35 (m, 5H), 1.24-1.2 (m, 1H), 1.11 (s, 3H), 1.05-0.94 (m, 7H), 0.82-0.59 (m, 2H) ppm. Chiral purity: 100%.

Example 115. Compound 115A and Compound 115B. Diastereomers 1 and 2 of 3-[3-(24,30-difluoro-11,11-dimethyl-10,26-dioxa-5,13,14,15,21,33-hexazahexacyclo[25.3.1.12,5.112,15.017,25.018,22]tritriaconta-1(31),2(33),3,12(32),13,17,19,22,24,27,29-undecaen-6-yl)phenyl]-2-methyl-propanoic acid

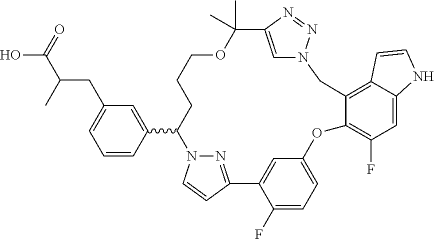 [SMILES: CC(Cc1cccc(C2CCCOC(C)(C)c3cn(nn3)Cc3c(c(F)cc4[nH]ccc34)Oc3ccc(F)c(c3)-c3ccn2n3)c1)C(=O)O]

Exchanging (5-(3-(1H-pyrazol-5-yl)phenoxy)-6-fluoro-1-tosyl-1H-indol-4-yl)methanol (Intermediate 55-4) with (6-fluoro-5-(4-fluoro-3-(1H-pyrazol-5-yl)phenoxy)-1-tosyl-1H-indol-4-yl)methanol (Intermediate 55-3, 3.5 g, 9.4 mmol), the reaction procedure sequence (Steps A to C) described for Example 100A and 100B was used to prepare the title compounds. The racemic ethyl ester (720 mg) was subject to chiral SFC separation using SFC-80 (Thar, Waters) under the following condition: Column: OJ 20*250 mm, 10 μm; Column temperature: 35° C.; Mobile phase: carbon dioxide/ethanol (0.5% methanol ammonia)=65/35; Flow rate: 80 g/minute; Back pressure: 100 bar; Detection wavelength: 214 nm; Cycle time: 5.7 minutes; Sample solution: 720 mg dissolved in 25 ml of methanol; Injection volume: 1.0 mL. The first eluent (275 mg, 38%), Diastereomer 1, was further hydrolyzed to Compound 115A (241 mg, 91%); The second eluent (290 mg, 40%), Diastereomer 2, was further hydrolyzed to Compound 115B (255 mg, 91%).

Compound 115A: MS (ESI): 667 m/z [M+H]⁺, retention time: 1.94 minutes, purity: 99% (214 nm) (LC-MS Method 26). ¹H NMR (400 MHz, DMSO-d₆) δ 12.17 (brs, 1H), 11.50 (brs, 1H), 7.79 (d, J=1.6 Hz, 1H), 7.50 (t, J=8 Hz, 1H), 7.40-7.38 (m, 1H), 7.34 (t, J=6.8 Hz, 1H), 7.29 (d, J=1.6 Hz, 1H), 7.26-7.21 (m, 2H), 7.17 (t, J=6 Hz, 1H), 7.07-7.05 (m, 2H), 6.99 (t, J=6 Hz, 1H), 6.58-6.56 (m, 1H), 6.10 (s, 1H), 5.94-5.90 (m, 1H), 5.80-5.77 (m, 1H), 5.32-5.29 (m, 1H), 3.19-3.18 (m, 1H), 3.01-2.98 (m, 1H), 2.86-2.79 (m, 1H), 2.57-2.53 (m, 2H), 2.49-2.40 (m, 1H), 1.71-1.68 (m, 1H), 1.41-1.37 (m, 1H), 1.34 (s, 3H), 1.30 (s, 3H), 1.06-1.03 (m, 1H), 0.96-0.90 (m, 3H) ppm.

Compound 115B: MS (ESI): 667 m/z [M+H]⁺, retention time: 1.94 minutes, purity: 97% (214 nm) (LC-MS Method 26). ¹H NMR (400 MHz, DMSO-d₆) δ 12.16 (brs, 1H), 11.52 (brs, 1H), 7.79 (d, J=2 Hz, 1H), 7.50 (t, J=8.8 Hz, 1H), 7.40-7.38 (m, 1H), 7.34 (t, J=7.2 Hz, 1H), 7.28 (d, J=1.2 Hz, 1H), 7.25-7.21 (m, 2H), 7.17 (t, J=5.6 Hz, 1H), 7.07-7.05 (m, 2H), 6.99 (t, J=6 Hz, 1H), 6.58-6.56 (m, 1H), 6.10 (s, 1H), 5.94-5.90 (m, 1H), 5.80-5.77 (m, 1H), 5.32-5.29 (m, 1H), 3.19-3.16 (m, 1H), 3.02-2.97 (m, 1H), 2.86-2.81 (m, 1H), 2.57-2.52 (m, 2H), 2.44-2.40 (m, 1H), 1.71-1.68 (m, 1H), 1.41-1.37 (m, 1H), 1.33 (s, 3H), 1.26 (s, 3H), 1.06-1.01 (m, 1H), 0.96-0.90 (m, 3H) ppm.

Example 116. Compound 116A and Compound 116B. Diastereomer 1 and 2 of 2-methyl-3-[3-(10,16,27,27-tetrafluoro-19,22-dimethyl-12-oxa-7,19,20,30,33-pentazahexacyclo-[26.2.2.113,17.118,21.03,11.04,8]tetratriaconta-1(30),3,5,8,10,13,15,17(34),18(33),20,28,31-dodecaen-22-yl)phenyl]propanoic acid

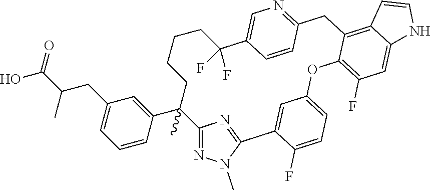 [SMILES: CC(Cc1cccc(C2(C)CCCCC(F)(F)c3ccc(nc3)Cc3c(c(F)cc4[nH]ccc34)Oc3ccc(F)c(c3)-c3nc2nn3C)c1)C(=O)O]

Exchanging methyl 2-fluoro-5-((6-fluoro-4-((4-(2-methylpent-4-en-2-yl)-1H-pyrazol-1-yl)methyl)-1-(phenylsulfonyl)-1H-indol-5-yl)oxy)benzimidothioate hydroiodide (Intermediate 82) with methyl 5-((4-((5-(1,1-difluorobut-3-en-1-yl)pyridin-2-yl)methyl)-6-fluoro-1H-indol-5-yl)oxy)-2-fluorobenzimidothioate hydroiodide (Intermediate 82-6, 2.8 g, 4.46 mmol)), the reaction procedure sequence (Steps A to H) described in Example 92A and 92B was used to prepare the title compound, with modifications on Step D. The Heck coupling in Step D was replaced by Step D conditions (Suzuki coupling) of Example 106. The racemic ethyl ester (330 mg), obtained in corresponding Step E, was subject to chiral SFC separation using SFC-80 (Thar, Waters) under the following conditions: Column: Whelk 20*250 mm, 10 μm; Column temperature: 35° C.; Mobile phase: carbon dioxide/methanol (0.2% methanol ammonia)=45/55; Flow rate: 80 g/minute; Back pressure: 100 bar; Detection wavelength: 214 nm; Cycle time: 4.3 minutes; Sample solution: 330 mg dissolved in 25 ml of methanol; Injection volume: 0.6 mL The first eluent (130 mg, 39%), Diastereomer 1, was further hydrolyzed to Compound 116A (110 mg, 88%); The second eluent (130 mg, 39%), Diastereomer 2, was further hydrolyzed to Compound 116B (113 mg, 90%).

Compound 116A: MS (ESI): 712 m/z [M+H]⁺, retention time: 1.95 minutes, purity: 95% (214 nm) (LC-MS Method 28). ¹H NMR (400 MHz, CD₃OD) δ 8.30 (s, 1H), 7.70 (d, J=8.0 Hz, 1H), 7.37-7.10 (m, 6H), 7.07-6.94 (m, 3H), 6.70-6.64 (m, 1H), 6.58 (d, J=3.2 Hz, 1H), 4.54-4.42 (m, 2H), 3.77 (s, 3H), 2.95-2.88 (m, 1H), 2.64-2.52 (m, 2H), 2.22-2.01 (m, 4H), 1.63 (s, 3H), 1.55-1.47 (m, 1H), 1.33-1.21 (m, 2H), 1.09-0.98 (m, 4H) ppm.

Compound 116B: MS (ESI): 712 m/z [M+H]⁺, retention time: 1.95 minutes, purity: 98% (214 nm) (LC-MS Method 28). ¹H NMR (400 MHz, CD₃OD) δ 8.31 (s, 1H), 7.71 (dd, J=8.4, 2 Hz, 1H), 7.36-7.10 (m, 6H), 7.07-6.95 (m, 3H), 6.70-6.64 (m, 1H), 6.58 (d, J=2.8 Hz, 1H), 4.54-4.42 (m, 2H), 3.77 (s, 3H), 2.95-2.88 (m, 1H), 2.64-2.52 (m, 2H), 2.22-2.01 (m, 4H), 1.63 (s, 3H), 1.55-1.47 (m, 1H), 1.33-1.21 (m, 2H), 1.08-0.98 (m, 4H) ppm.

Example 117. Compound 117A and 117B. Diastereomers 1 and 2 of (2S)-2-methyl-3-[3-(9,9,24,30-tetrafluoro-3,6-dimethyl-11,26-dioxa-3,4,15,21,32,33-hexazahexacyclo-[25.3.1.12,5.112,15.017,25.018,22]tritriaconta-1(31),2(33),4,12(32),13,17,19,22,24,27,29-undecaen-6-yl)phenyl]propanoic acid

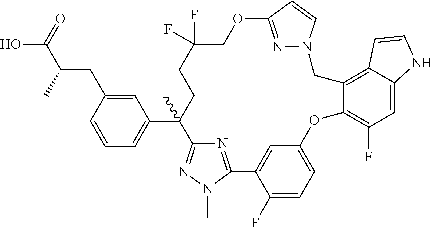 [SMILES: C[C@@H](Cc1cccc(C2(C)CCC(F)(F)COc3ccn(n3)Cc3c(c(F)cc4[nH]ccc34)Oc3ccc(F)c(c3)-c3nc2nn3C)c1)C(=O)O]

Exchanging ethyl 3-(3-(6-((1H-pyrazol-3-yl)oxy)-2,5,5-trimethyl-1-(2-methylhydrazineyl)-1-oxohexan-2-yl)phenyl)-2-methylpropanoate (Intermediate 83-5) with methyl (2S)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (Intermediate 101, 1.5 g, 3.3 mmol), the reaction procedure sequence (Steps A to E) described for Example 99A & 99B, 99A & 99B was used to prepare the title compounds. The phenylsulfonyl protected methyl ester (350 mg), obtained from corresponding Step D, was subject to chiral SFC separation under the following condition: Instrument: SFC-150; Column: R′R WHELK 20*250 mm, 10 μm; Column temperature: 35° C.; Mobile phase: carbon dioxide/methanol (0.2% methanol ammonia)=40/60; Flow rate: 120 g/minute; Back pressure: 100 bar; Detection wavelength: 214 nm; Cycle time: 10 minutes; Sample solution: 350 mg dissolved in 40 mL of methanol and dichloromethane; Injection volume: 4 mL. The first eluent (120 mg, 37%), Diastereomer 1, was further deprotected and hydrolyzed to Compound 117A (81.7 mg, 83%); The second eluent (114 mg, 35%), Diastereomer 2, was further deprotected and hydrolyzed to Compound 117B (88.5 mg, 94%), as described in Step E of Example 99A & 99B, 99A & 99B.

Compound 117A: MS (ESI): 703 m/z [M+H]⁺, retention time: 1.90 minutes, purity: >99% (214 nm) (LC-MS Method 28). ¹H NMR (400 MHz, CD₃OD) δ 7.39-7.31 (m, 2H), 7.28 (d, J=2.0 Hz, 1H), 7.25-7.20 (m, 2H), 7.15 (t, J=7.6 Hz, 1H), 7.03-6.98 (m, 3H), 6.61 (d, J=2.8 Hz, 1H), 6.30 (dd, J=6.4, 3.2 Hz, 1H), 5.47-3.34 (m, 3H), 4.14-4.06 (m, 1H), 3.89-3.83 (m, 1H), 3.71 (d, J=1.2 Hz, 3H), 2.97-2.90 (m, 1H), 2.63-2.54 (m, 2H), 2.30-2.14 (m, 2H), 2.09-1.96 (m, 1H), 1.80-1.66 (m, 1H), 1.61 (s, 3H), 1.04 (d, J=6.4, Hz, 3H) ppm.

Compound 117B: MS (ESI): 703 m/z [M+H]⁺, retention time: 1.90 minutes, purity: >99% (214 nm) (LC-MS Method 28). ¹H NMR (400 MHz, CD₃OD) δ 7.38-7.31 (m, 2H), 7.28 (d, J=2.4 Hz, 1H), 7.25-7.22 (m, 2H), 7.15 (t, J=7.6 Hz, 1H), 7.03-6.99 (m, 3H), 6.61 (d, J=3.2 Hz, 1H), 6.30 (dd, J=5.6, 3.2 Hz, 1H), 5.47-3.34 (m, 3H), 4.13-4.05 (m, 1H), 3.90-3.81 (m, 1H), 3.71 (d, J=1.6 Hz, 3H), 2.95-2.88 (m, 1H), 2.60-2.53 (m, 2H), 2.29-2.15 (m, 2H), 2.09-1.97 (m, 1H), 1.81-1.66 (m, 1H), 1.60 (s, 3H), 1.03 (d, J=6.8, Hz, 3H) ppm.

Example 118. Compound 118A and Compound 118B. Regio isomers 1 of (2S)-2-methyl-3 [3-(9,9,24,30-tetrafluoro-6-methyl-11,26-dioxa-3,15,21,32,33-pentazahexacyclo-[25.3.1.12,5.112,15.017,25.018,22]tritriaconta-1(31),2(33),4,12(32),13,17,19,22,24,27,29-undecaen-6-yl)phenyl]propanoic acid

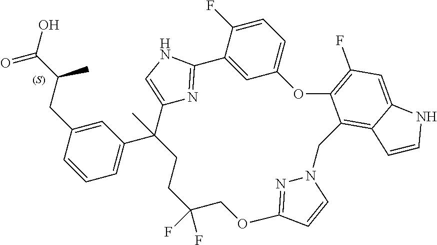

and

Regio Isomer 2 of (2S)-2-methyl-3-[3-(9,9,25,31-tetrafluoro-6-methyl-11,27-dioxa-3,15,16,22,33-pentazahexacyclo[26.3.1.12,5.012,16.018,26.019,23]-tritriaconta-1(32),2(33),4,12,14,18,20,23,25,28,30-undecaen-6-yl)phenyl]propanoic acid

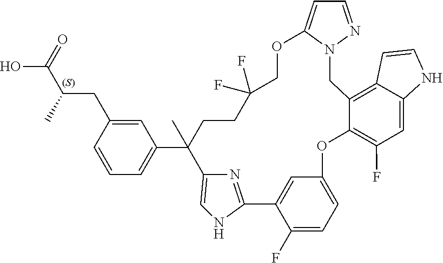

Methyl (2S)-3-(3-(6-((1-acetyl-1H-pyrazol-3-yl)oxy)-5,5-difluoro-2-(2-(2-fluoro-5-((6-fluoro-4-(hydroxymethyl)-1-(phenylsulfonyl)-1H-indol-5-yl)oxy)phenyl)-1H-imidazol-4-yl)hexan-2-yl)phenyl)-2-methylpropanoate

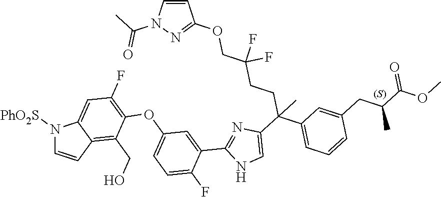

Step A: To a solution of 2-fluoro-5-((6-fluoro-4-(hydroxymethyl)-1-(phenylsulfonyl)-1H-indol-5-yl)oxy)benzimidamide (Intermediate 6-14, 757 mg, 1.66 mmol) in N,N-dimethylformamide (10 mL) was added methyl (2S)-3-(3-(7-((1-acetyl-1H-pyrazol-3-yl)oxy)-1-bromo-6,6-difluoro-3-methyl-2-oxoheptane-3-yl)phenyl)-2-methylpropanoate (Intermediate 2-50, 750 mg, 1.38 mmol) and sodium bicarbonate (232 mg, 2.76 mmol). The reaction was stirred at 75° C. for 16 hours, cooled to room temperature and diluted with ethyl acetate (100 mL). The solution was washed with brine, dried over sodium sulfate, and concentrated. The residue was purified by flash chromatography (24 g silica gel column, eluting with 0-50% ethyl acetate in petroleum ether) to give the title compound (1.2 g, 80%) as a solid. MS (ESI): 902 m/z [M+H]⁺, retention time: 1.84 minutes, purity: 55% (254 nm) (LC-MS Method 5).

Compounds 118A and 118B: Regio isomers 1 of (2S)-2-methyl-3-[3-(9,9,24,30-tetrafluoro-6-methyl-11,26-dioxa-3,15,21,32,33-pentazahexacyclo-[25.3.1.12,5.112,15.017,25.018,22]tritriaconta-1(31),2(33),4,12(32),13,17,19,22,24,27,29-undecaen-6-yl)phenyl]propanoic acid

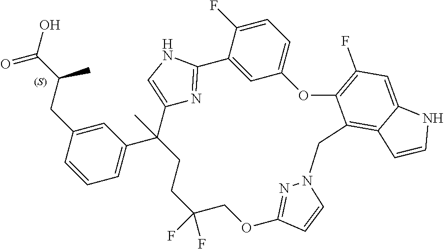

and Regio-isomer 2 of (2S)-2-methyl-3-[3-(9,9,25,31-tetrafluoro-6-methyl-11,27-dioxa-3,15,16,22,33-pentazahexacyclo[26.3.1.12,5.012,16.018,26.019,23]-tritriaconta-1(32),2(33),4,12,14,18,20,23,25,28,30-undecaen-6-yl)phenyl]propanoic acid

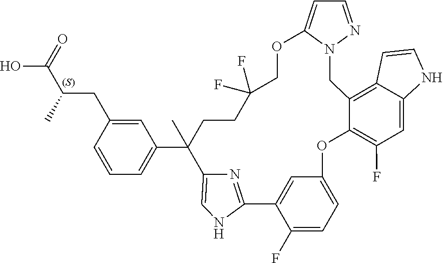

Step B: Exchanging ethyl 3-(3-(6-((1H-pyrazol-3-yl)oxy)-2-(5-(5-((4-(acetoxymethyl)-6-fluoro-1-(phenylsulfonyl)-1H-indol-5-yl)oxy)-2-fluorophenyl)-1-methyl-1H-1,2,4-triazol-3-yl)-5,5-dimethylhexan-2-yl)phenyl)-2-methylpropanoate (Step A product of Example 99A & 99B, 99A & 99B) with Methyl (2S)-3-(3-(6-((1-acetyl-1H-pyrazol-3-yl)oxy)-5,5-difluoro-2-(2-(2-fluoro-5-((6-fluoro-4-(hydroxymethyl)-1-(phenylsulfonyl)-1H-indol-5-yl)oxy)phenyl)-1H-imidazol-4-yl)hexan-2-yl)phenyl)-2-methylpropanoate (Step A product of this example, 1.2 g, 1.33 mmol), the reaction procedure sequence (Steps B, C and E) described for Example 99A & 99B, 99A & 99B was used to prepare the title compounds. The formed two regioisomers were not separable at corresponding Step C, and instead, they were separated by Prep-HPLC at corresponding Step E, after deprotection and ester hydrolysis. The first eluent, Regioisomer A, was designated as Compound 118A (31.4 mg); The second eluent, Regioisomer B, was designated as Compound 118B (16.9 mg).

Compound 118A: MS (ESI): 688 m/z [M+H]⁺, retention time: 1.56 minutes, purity: >99% (254 nm) (LC-MS Method 5). ¹H NMR (400 MHz, CD₃OD) δ 7.33 (d, J=3.2 Hz, 1H), 7.25-7.18 (m, 5H), 7.15-7.11 (m, 1H), 6.99-6.97 (m, 3H), 6.68 (d, J=3.2 Hz, 1H), 6.60-6.59 (m, 1H), 5.45-5.33 (m, 2H), 5.18 (d, J=2.4 Hz, 1H), 4.26-4.16 (m, 1H), 3.83-3.76 (m, 1H), 2.92-2.85 (m, 1H), 2.60-2.52 (m, 2H), 2.31-2.25 (m, 1H), 2.17 (t, J=12.4 Hz, 1H), 2.02-1.92 (m, 1H), 1.72-1.64 (m, 1H), 1.51 (s, 3H), 1.05-1.01 (m, 3H) ppm.

Compound 118B: MS (ESI): 688 m/z [M+H]⁺, retention time: 1.62 minutes, purity: >99% (254 nm) (LC-MS Method 5). ¹H NMR (400 MHz, CD₃OD) δ 7.33 (d, J=2.4 Hz, 1H), 7.30-7.28 (m, 1H), 7.27-7.24 (m, 2H), 7.16 (d, J=10.4 Hz, 1H), 7.11-7.07 (m, 2H), 7.02 (d, J=6.0 Hz, 1H), 6.98-6.94 (m, 3H), 5.82 (d, J=2.0 Hz, 1H), 5.60-5.50 (m, 2H), 5.03 (d, J=3.2 Hz, 1H), 4.08-4.02 (m, 2H), 2.91-2.84 (m, 1H), 2.57-2.53 (m, 2H), 2.36-2.27 (m, 2H), 1.99-1.91 (m, 2H), 1.56 (s, 3H), 1.03-0.99 (m, 3H) ppm.

Example 119. Compound 119. (2S)-2-Methyl-3-[3-(9,9,24,30-tetrafluoro-3,6-dimethyl-26-oxo-11-oxa-3,4,15,21,32,33-hexazahexacyclo[25.3.1.12,5.112,15.017,25.018,22]tritriaconta-1(31),2(33),4,12(32),13,17,19,22,24,27,29-undecaen-6-yl)phenyl]propanoic acid

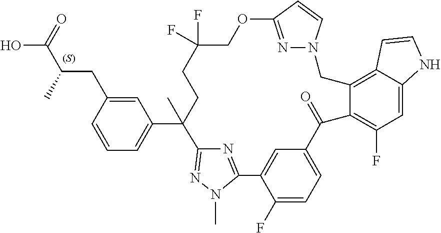

Methyl (2S)-3-(3-(6-((1H-pyrazol-3-yl)oxy)-5,5-difluoro-2-(5-(2-fluoro-5-((6-fluoro-1-(triisopropylsilyl)-4-vinyl-1H-indol-5-yl)((tetrahydro-2H-pyran-2-yl)oxy)methyl)phenyl)-1-methyl-1H-1,2,4-triazol-3-yl)hexan-2-yl)phenyl)-2-methylpropanoate

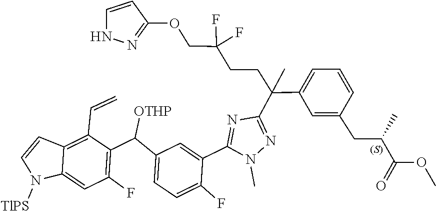

Step A: To a stirred solution of methyl 2-fluoro-5-((6-fluoro-1-(triisopropylsilyl)-4-vinyl-1H-indol-5-yl)((tetrahydro-2H-pyran-2-yl)oxy)methyl)benzimidothioate hydroiodide (Intermediate 82-7, 0.466 g, 1.03 mmol) in pyridine (20 mL) was added methyl (2S)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (Intermediate 101, 1.5 g, 2.06 mmol) and magnesium sulphate (1.5 g). The reaction mixture was stirred at 80° C. for 3 hours, cooled to room temperature, and diluted with ethyl acetate (60 mL). The solution was washed with brine, dried over sodium sulfate, and concentrated. The residue was purified by automated flash chromatography (40 g silica gel column, eluting with 0-70% ethyl acetate in petroleum ether) to give the title compound (0.38 g, 38%) as a solid. MS (ESI): 985.5 m/z [M+H]⁺, retention time: 2.73 and 2.80 minutes (two separable diastereomers), purity: 20% and 44% (two separable diastereomers) (254 nm) (LC-MS Method 26).

Methyl (2S)-3-(3-(6-((1H-pyrazol-3-yl)oxy)-5,5-difluoro-2-(5-(2-fluoro-5-((6-fluoro-4-formyl-1-(triisopropylsilyl)-1H-indol-5-yl)((tetrahydro-2H-pyran-2-yl)oxy)methyl)phenyl)-1-methyl-1H-1,2,4-triazol-3-yl)hexan-2-yl)phenyl)-2-methylpropanoate

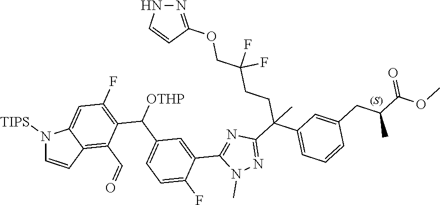

Step B: To a stirred solution of Step A product (370 mg, 0.38 mmol) in tetrahydrofuran/water (40 mL) was added sodium metaperiodate (247 mg, 1.15 mmol) and osmium(VIII) oxide (2 mg/mL in water, 10 mL), the mixture was stirred at room temperature overnight, quenched with saturated sodium dithionite (50 mL). The solution was diluted with ethyl acetate (100 mL). The separated organic layer was washed with brine, dried over sodium sulphate, and concentrated. The residue was purified by silica gel column (20 g silica gel column, eluting with 0-80% ethyl acetate in petroleum ether) to give the title compound (190 mg, 49%) as solid. MS (ESI): 987.5 m/z [M+H]⁺, retention time: 2.51 minutes, purity: 24% and 65% (two separable diastereomers) (214 nm) (LC-MS Method 26).

Methyl (2S)-3-(3-(6-((1H-pyrazol-3-yl)oxy)-5,5-difluoro-2-(5-(2-fluoro-5-((6-fluoro-4-(hydroxymethyl)-1-(triisopropylsilyl)-1H-indol-5-yl)((tetrahydro-2H-pyran-2-yl)oxy)methyl)phenyl)-1-methyl-1H-1,2,4-triazol-3-yl)hexan-2-yl)phenyl)-2-methylpropanoate

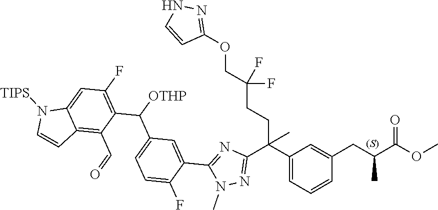

Step C: To a stirred Step B product (190 mg, 0.2 mmol) in methanol (20 mL) was added sodium borohydride (16 mg, 0.4 mmol). The mixture was stirred at room temperature for 2 hours. The mixture was quenched with water and extracted with ethyl acetate (100 mL). The separated organic layer was washed with brine, dried over sodium sulphate, and concentrated. The residue was purified by automated flash chromatography (40 g silica gel column, eluting with 0-50% ethyl acetate in petroleum) to give the title compound (90 mg, 50%) as solid. MS (ESI): 989.4 m/z [M+H]⁺, retention time: 2.46 minutes, purity: 89%) (214 nm) (LC-MS Method 26).

Methyl (2S)-2-methyl-3-[3-(9,9,24,30-tetrafluoro-3,6-dimethyl-26-tetrahydropyran-2-yloxy-11-oxa-3,4,15,21,32,33-hexazahexacyclo[25.3.1.12,5.112,15.017,25.018,22]tritriaconta-1(31),2(33),4,12(32),13,17,19,22,24,27,29-undecaen-6-yl)phenyl]propanoate

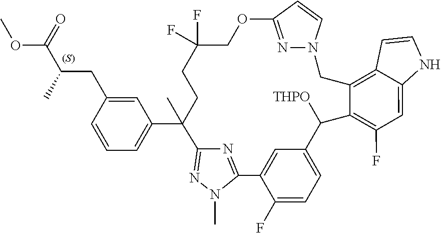

Step D: To a solution of Step C product (90 mg, 0.09 mmol) in toluene (10 mL) was added cynomethylenetributyl phosphorane (CAS:157141-27-01, 110 mg, 0.45 mmol). The reaction mixture was stirred at 150° C. for 45 minutes in a microwave reactor, then concentrated. The residue was purified by automated flash chromatography (20 g silica gel column, eluting with 0-30% ethyl acetate in petroleum ether) to give the title compound (20 mg, 27%) as a solid. MS (ESI): 815.3 m/z [M+H]⁺, retention time: 2.17 and 2.20 minutes (two separable diastereomers), purity: 59+27% (two separable diastereomers) (214 nm) (LC-MS Method 26).

Methyl (2S)-2-methyl-3-[3-(9,9,24,30-tetrafluoro-26-hydroxy-3,6-dimethyl-11-oxa-3,4,15,21,32,33-hexazahexacyclo[25.3.1.12,5.112,15.017,25.018,22]tritriaconta-1(31),2(33),4,12(32),13,17,19,22,24,27,29-undecaen-6-yl)phenyl]propanoate

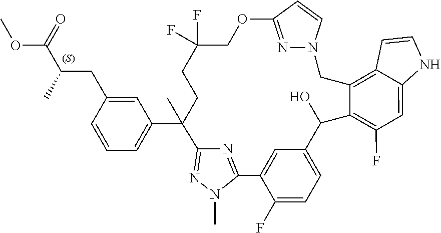

Step E: To a stirred solution of Step D product (20 mg, 0.025 mmol) in methanol (10 mL) was added pyridinium 4-methylbenzenesulfonate (27 mg, 0.11 mmol). The reaction was stirred at 60° C. for 1 hour. The mixture was diluted with ethyl acetate (40 mL). The solution was washed with brine, dried over magnesium sulfate, and concentrated to give the title compound (17.9 mg, 100%) as a solid. MS (ESI): 731.4 m/z [M+H]⁺, retention time: 1.99 and 2.00 minutes (two separable diastereomers), purity: 41+39% (two separable diastereomers) (214 nm) (LC-MS Method 26).

Methyl (2S)-2-methyl-3-[3-(9,9,24,30-tetrafluoro-3,6-dimethyl-26-oxo-11-oxa-3,4,15,21,32,33-hexazahexacyclo[25.3.1.12,5.112,15.017,25.018,22]tritriaconta-1(31),2(33),4,12(32),13,17,19,22,24,27,29-undecaen-6-yl)phenyl]propanoate

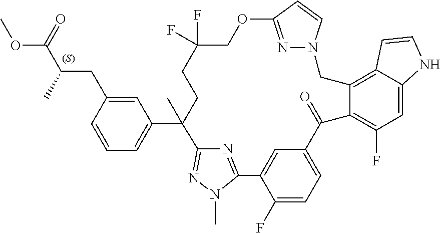

Step F: To a stirred solution of Step E product (17.9 mg, 0.025 mmol) in dimethyl sulfoxide (4 mL) was added 2-iodoxybenzoic acid (20 mg, 0.075 mmol). The mixture was stirred at room temperature for two hours, then diluted with ethyl acetate (40 mL). The solution was washed with saturated sodium bicarbonate (20 mL), dried over anhydrous sodium sulfate, and concentrated. The residue was purified by automated flash chromatography (12 g silica gel column, eluting with 0-60% ethyl acetate in petroleum ether) to give the title compound (10 mg, 56%) as a solid. MS (ESI): 729.3 m/z [M+H]⁺.

Example 119. (2S)-2-Methyl-3-[3-(9,9,24,30-tetrafluoro-3,6-dimethyl-26-oxo-11-oxa-3,4,15,21,32,33-hexazahexacyclo[25.3.1.12,5.112,15.017,25.018,22]tritriaconta-1(31),2(33),4,12(32),13,17,19,22,24,27,29-undecaen-6-yl)phenyl]propanoic acid

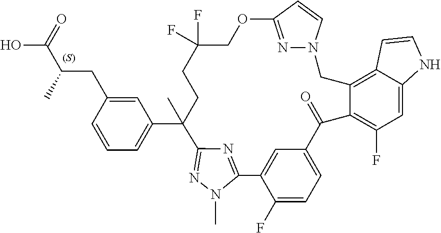

Step G: To a stirred solution of Step F product (10 mg, 0.014 mmol) in tetrahydrofuran/water (6 mL/2 mL) was added lithium hydroxide (2.8 mg, 0.068 mmol). The reaction was stirred at room temperature overnight, then acidified with 1N hydrochloric acid to pH ˜4 and diluted with ethyl acetate (20 mL). The solution was washed with brine, dried over sodium sulphate, and concentrated. The crude product was purified by prep-HPLC to give the title compound (3.4 mg, 35%) as a white solid. MS (ESI): 715 m/z [M+H]⁺, retention time: 1.86 minutes, purity: >99% (214 nm) (LC-MS Method 26). ¹H NMR (400 MHz, CD₃OD) δ 8.20-8.13 (m, 1H), 7.45 (t, J=8.8 Hz, 1H), 7.39-7.33 (m, 2H), 7.20-7.15 (m, 3H), 7.06-6.98 (m, 3H), 6.71 (d, J=3.6 Hz, 1H), 5.44 (s, 2H), 5.31 (d, J=2.4 Hz, 1H), 4.16-4.05 (m, 1H), 3.86-3.78 (m, 1H), 3.74 (s, 3H), 3.00-2.90 (m, 1H), 2.64-2.53 (m, 2H), 2.34-2.27 (m, 1H), 2.22-2.13 (m, 1H), 2.09-1.96 (m, 1H), 1.78-1.68 (m, 1H), 1.64 (s, 3H), 1.05 (d, J=6.4 Hz, 3H) ppm.

Example 120. Compound 120A and Compound 120B. Diastereomers 1 and 2 of (2R)-2-methyl-3-[3-(9,9,24,30-tetrafluoro-3,6-dimethyl-11,26-dioxa-3,4,15,21,32,33-hexazahexacyclo-[25.3.1.12,5.112,15.017,25.018,22]tritriaconta-1(31),2(33),4,12(32),13,17,19,22,24,27,29-undecaen-6-yl)phenyl]propanoic acid

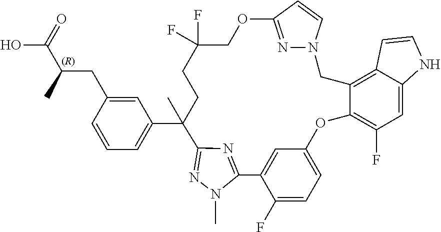

Exchanging ethyl 3-(3-(6-((1H-pyrazol-3-yl)oxy)-2,5,5-trimethyl-1-(2-methylhydrazineyl)-1-oxohexan-2-yl)phenyl)-2-methylpropanoate (Intermediate 83-5) with methyl (2R)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (Intermediate 97-1, 1 g, 2.2 mmol), the reaction procedure sequence (Steps A to E) described for Example 99A & 99B, 99A & 99B was used to prepare the title compounds, with slight modification. Only one major regioisomer was obtained, another minor one was lost. The chiral separation (corresponding Step D) was performed after deprotection and hydrolysis (corresponding Step E). The chiral separation conditions for the racemic acid (276 mg), obtained after deprotection and hydrolysis, was as follows: Instrument: SFC-80 (Thar, Waters); Column: WHELK 20*250 mm, 10 μm; Column temperature: 35° C.; Mobile phase: carbon dioxide/methanol (0.2% methanol ammonia)=45/55; Flow rate: 80 g/minute; Back pressure: 100 bar; Detection wavelength: 214 nm; Cycle time: 7.5 minutes; Sample solution: 276 mg dissolved in 20 mL of methanol; Injection volume: 1.9 mL. The first eluent (103.9 mg, 38%), Diastereomer 1, was designated as Compound 120A; The second eluent (118 mg, 43%), Diastereomer 2, was designated as Compound 120B.

Compound 120A: MS (ESI): 703 m/z [M+H]⁺, retention time: 2.14 minutes, purity: >99% (254 nm) (LC-MS Method 5). ¹H NMR (400 MHz, CD₃OD) δ 7.36-7.33 (m, 1H), 7.31 (d, J=3.2 Hz, 1H), 7.28 (d, J=2.4 Hz, 1H), 7.25-7.20 (m, 2H), 7.15 (t, J=8.4 Hz, 1H), 7.03-6.98 (m, 3H), 6.61 (d, J=2.8 Hz, 1H), 6.29 (dd, J=6.4, 3.2 Hz, 1H), 5.42 (q, J=24.4, 14.0 Hz, 2H), 5.36 (d, J=2.4 Hz, 1H), 4.15-4.06 (m, 1H), 3.85 (q, J=20.4, 10.4 Hz, 1H), 3.68 (d, J=1.6 Hz, 3H), 2.96-2.90 (m, 1H), 2.62-2.53 (m, 2H), 2.30-2.14 (m, 2H), 2.05-1.98 (m, 1H), 1.81-1.66 (m, 1H), 1.61 (s, 3H), 1.04 (d, J=6.8, Hz, 3H) ppm.

Compound 120B: MS (ESI): 857 m/z [M+H]⁺, retention time: 2.14 minutes, purity: >99% (254 nm) (LC-MS Method 5). ¹H NMR (400 MHz, CD₃OD) δ 7.37-7.33 (m, 1H), 7.31 (d, J=3.2 Hz, 1H), 7.28 (d, J=2.4 Hz, 1H), 7.25-7.22 (m, 2H), 7.15 (t, J=8.0 Hz, 1H), 7.03-6.98 (m, 3H), 6.61 (d, J=2.8 Hz, 1H), 6.29 (dd, J=6.4, 3.2 Hz, 1H), 5.47-5.35 (m, 3H), 4.15-4.05 (m, 1H), 3.89-3.81 (m, 1H), 3.68 (d, J=1.6 Hz, 3H), 2.94-2.89 (m, 1H), 2.60-2.53 (m, 2H), 2.28-2.15 (m, 2H), 2.08-1.98 (m, 1H), 1.82-1.67 (m, 1H), 1.60 (s, 3H), 1.03 (d, J=6.8, Hz, 3H) ppm.

Example 121. Compound 121B, Compound 121C, and Compound 121A. Diastereomers 1 and 2 of major Regio-isomer 1 of (2R)-2-methyl-3-[3-[(6R)-9,9,24,30-tetrafluoro-3,6-dimethyl-26-oxo-11-oxa-3,4,15,21,32,33-hexazahexacyclo-[25.3.1.12,5.112,15.017,25.018,22]tritriaconta-1(31),2(33),4,12(32),13,17,19,22,24,27,29-undecaen-6-yl]phenyl]propanoic acid

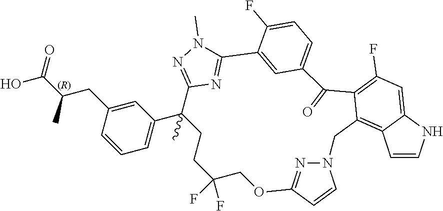

and

Example 121A. Minor Regio-isomer 2, (2R)-2-methyl-3-[3-(9,9,25,31-tetrafluoro-3,6-dimethyl-27-oxo-11-oxa-3,4,15,16,22,33-hexazahexacyclo[26.3.1.12,5.012,16.018,26.019,23]-tritriaconta-1(32),2(33),4,12,14,18,20,23,25,28,30-undecaen-6-yl)phenyl]propanoic acid

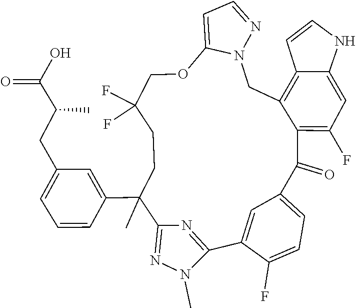 [SMILES: C[C@H](Cc1cccc(C2(C)CCC(F)(F)COc3ccnn3Cc3c(c(F)cc4[nH]ccc34)C(=O)c3ccc(F)c(c3)-c3nc2nn3C)c1)C(=O)O]

Exchanging methyl (2S)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (Intermediate 101) with methyl (2R)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (intermediate 97-1, 0.937 g, 2.06 mmol), the reaction procedure sequence (Steps A to G) described for Example 119 was used to prepare the title compounds, with modifications. The TIPS protecting group stayed on after macrocyclization (Step C of Example 119), so one additional deprotection step (following conditions described in Intermediate 12A) after macrocyclization was necessary. Two regioisomers were separated and collected at corresponding Step F, the oxidation of alcohol to ketone. The minor regioisomer, Regioisomer 2 (30 mg), was further hydrolyzed to Compound 121A (14 mg, 49%), following conditions described in corresponding Step G.

The major regioisomer, Regioisomer 1 (150 mg), was subject to chiral SFC separation using the following conditions: Instrument: SFC-150 (Waters); Column: R′R WHELK 20*250 mm, 10 μm; Column temperature: 35° C.; Mobile phase: carbon dioxide/(Methanol/acetonitrile (1:1)(0.2% methanol ammonia))=50/50; Flow rate: 100 g/minute; Back pressure: 100 bar; Detection wavelength: 214 nm; Cycle time: 3 minutes; Sample solution: 120 mg dissolved in 20 mL methanol; Injection volume: 2 mL. The first eluent, Diastereomer 1 (56 mg, 37%), was further hydrolyzed to Compound 121B (41 mg, 74%). The second eluent, Diastereomer 2 (60 mg, 40%), was hydrolyzed to Compound 121C (38 mg, 65%), following the conditions described in Step G of Example 119.

Compound 121A: MS (ESI): 715 m/z [M+H]⁺, retention time: 1.59 minutes, purity: >99% (214 nm) (LC-MS Method 8). ¹H NMR (400 MHz, CD₃OD) δ 8.36-8.31 (m, 1H), 7.60-7.52 (m, 2H), 7.20 (d, J=3.6 Hz, 1H), 7.15-7.09 (m, 3H), 6.70-6.88 (m, 3H), 5.69-5.67 (m, 2H), 5.50-5.39 (m, 2H), 4.13-4.07 (m, 2H), 3.84 (d, J=2.4 Hz, 3H), 2.93-2.83 (m, 1H), 2.61-2.50 (m, 2H), 2.41-2.26 (m, 2H), 2.05-1.97 (m, 1H), 1.76-1.61 (m, 4H), 1.04-1.00 (m, 3H) ppm.

Compound 121B: MS (ESI): 715 m/z [M+H]⁺, retention time: 1.95 minutes, purity: >99% (214 nm) (LC-MS Method 26). ¹H NMR (400 MHz, CD₃OD) δ 8.19-8.14 (m, 1H), 7.43 (t, J=11.2 Hz, 1H), 7.37-7.34 (m, 2H), 7.19-7.15 (m, 3H), 7.06-7.01 (m, 3H), 6.70 (d, J=3.2 Hz, 1H), 5.43 (s, 2H), 5.31 (d, J=2.4 Hz, 1H), 4.15-4.05 (m, 1H), 3.87-3.78 (m, 1H), 3.74 (s, 3H), 2.96-2.92 (m, 1H), 2.66-2.53 (m, 2H), 2.34-1.99 (m, 3H), 1.77-1.62 (m, 4H), 1.05 (d, J=6.4 Hz, 3H) ppm. Chiral-HPLC:99.7% (Chiral HPLC condition: Column: (R,R)Whelk-O1, 4.6×100 mm, 3.5 μm; Mobile phase: carbon dioxide/methanol (0.2% (7M ammonia in methanol))=55:45; Run time: 6 minutes; Flow rate: 3 mL/minute; Back pressure: 2000 psi.)

Compound 121C: MS (ESI): 715 m/z [M+H]⁺, retention time: 1.95 minutes, purity: >99% (214 nm) (LC-MS Method 26). ¹H NMR (400 MHz, CD₃OD) δ 8.19-8.14 (m, 1H), 7.44 (t, J=9.2 Hz, 1H), 7.38-7.35 (m, 2H), 7.19-7.14 (m, 3H), 7.05-7.00 (m, 3H), 6.70 (d, J=2.8 Hz, 1H), 5.43 (s, 2H), 5.31 (d, J=2.0 Hz, 1H), 4.16-4.06 (m, 1H), 3.87-3.78 (m, 1H), 3.74 (s, 3H), 2.98-2.92 (m, 1H), 2.62-2.53 (m, 2H), 2.34-1.98 (m, 3H), 1.77-1.68 (m, 1H), 1.64 (s, 3H), 1.05 (d, J=6.8 Hz, 3H) ppm. Chiral-HPLC: 100% (Chiral HPLC condition: Column: (R,R)Whelk-O1, 4.6×100 mm, 3.5 μm; Mobile phase: carbon dioxide/methanol (0.2% (7M ammonia in methanol))=55:45; Run time: 6 minutes; Flow rate: 3 mL/minute; Back pressure: 2000 psi. Retention time: 1.37 minutes).

Example 122. Compound 122. 1-[[3-(9,9,24,30-tetrafluoro-3,6-dimethyl-26-oxo-11-oxa-3,4,15,21,32,33-hexazahexacyclo[25.3.1.12,5.112,15.017,25.018,22]tritriaconta-1(31),2(33),4,12(32),13,17,19,22,24,27,29-undecaen-6-yl)phenyl]methyl]-cyclopropanecarboxylic acid

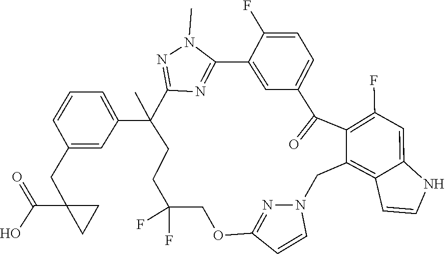 [SMILES: Cn1nc2nc1-c1cc(ccc1F)C(=O)c1c(F)cc3[nH]ccc3c1Cn1ccc(n1)OCC(F)(F)CCC2(C)c1cccc(CC2(C(=O)O)CC2)c1]

and

Example 123. Compound 122-22. Methyl 1-[[3-(9,9,24,30-tetrafluoro-3,6-dimethyl-26-oxo-11-oxa-3,4,15,21,32,33-hexazahexacyclo[25.3.1.12,5.112,15.017,25.018,22]tritriaconta-1(31),2(33),4,12(32),13,17,19,22,24,27,29-undecaen-6-yl)phenyl]methyl]-cyclopropanecarboxylate

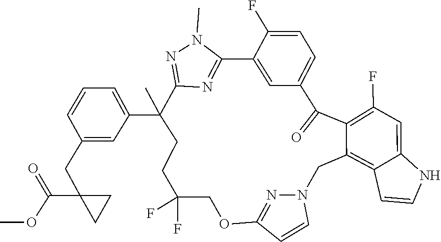 [SMILES: COC(=O)C1(Cc2cccc(C3(C)CCC(F)(F)COc4ccn(n4)Cc4c(c(F)cc5[nH]ccc45)C(=O)c4ccc(F)c(c4)-c4nc3nn4C)c2)CC1]

Exchanging methyl (2S)-3-(3-(6-((1H-pyrazol-3-yl)oxy)-5,5-difluoro-2-methyl-1-(2-methyl-hydrazineyl)-1-oxohexan-2-yl)phenyl)-2-methylpropanoate (Intermediate 101) with methyl 1-(3-(6-((1H-pyrazol-3-yl)oxy)-5,5-difluoro-2-methyl-1-(2-methylhydrazineyl)-1-oxohexan-2-yl)benzyl)cyclopropane-1-carboxylate (intermediate 97-2, 1.3 g, 2.78 mmol), the reaction procedure sequence (Steps A to G) described for Example 119 was used to prepare the title compounds, with modifications. The TIPS protecting group stayed on after macrocyclization (Step C of Example 119), so one additional TBAF deprotection step (following conditions described in Intermediate 12A) after THP deprotection (Step E). Only one regio-isomer was separated and collected at corresponding Step F, the oxidation of alcohol to ketone (22 mg, designated as Compound 122-22). The ketone was further hydrolyzed to Compound 122 (3.7 mg, 17%), following conditions described in corresponding Step E.

Compound 122: MS (ESI): 727 m/z [M+H]⁺, retention time: 1.69 minutes, purity: 97% (214 nm) (LC-MS Method 8). ¹H NMR (400 MHz, CD₃OD) δ 8.19-8.14 (m, 1H), 7.45 (t, J=9.2 Hz, 1H), 7.38-7.35 (m, 2H), 7.18-7.14 (m, 3H), 7.08-7.04 (m, 3H), 6.71 (d, J=3.2 Hz, 1H), 5.44 (s, 2H), 5.30 (d, J=2.4 Hz, 1H), 4.15-4.05 (m, 1H), 3.87-3.74 (m, 4H), 2.95-2.85 (m, 2H), 2.33-2.27 (m, 1H), 2.18-1.96 (m, 2H), 1.70-1.63 (m, 4H), 1.16-0.10 (m, 2H), 0.73-0.65 (m, 2H) ppm.

Compound 122-22: MS (ESI): 741 m/z [M+H]⁺, retention time: 2.09 minutes, purity: >99% (214 nm) (LC-MS Method 26). ¹H NMR (400 MHz, CD₃OD) δ 8.20-8.15 (m, 1H), 7.48-7.43 (m, 1H), 7.39-7.36 (m, 2H), 7.18-7.14 (m, 3H), 7.09-7.02 (m, 3H), 6.71 (d, J=2.8 Hz, 1H), 5.44 (s, 2H), 5.29 (d, J=2.4 Hz, 1H), 4.15-4.04 (m, 1H), 3.89-3.81 m, 1H), 3.76 (d, J=1.6 Hz, 3H), 3.51 (s, 3H), 2.89 (s, 2H), 2.31-2.25 (m, 1H), 2.18-2.00 (m, 2H), 1.76-1.69 (m, 1H), 1.64 (s, 3H), 1.16-0.10 (m, 2H), 0.83-0.71 (m, 2H) ppm.

Example 124. Compound 124A and Compound 124B. (2R)-2-methyl-3-[3-(9,9,24,30-tetrafluoro-3,6-dimethyl-26-oxo-11-oxa-26λ4-thia-3,4,15,21,32,33-hexazahexacyclo[25.3.1.12,5.112,15.017,25.018,22]tritriaconta-1(31),2(33),4,12(32),13,17,19,22,24,27,29-undecaen-6-yl)phenyl]propanoic acid

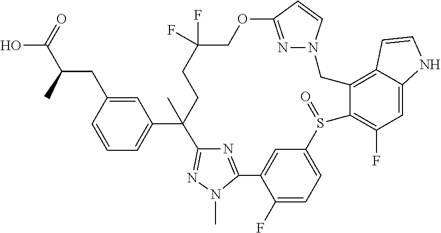 [SMILES: C[C@H](Cc1cccc(C2(C)CCC(F)(F)COc3ccn(n3)Cc3c(c(F)cc4[nH]ccc34)S(=O)c3ccc(F)c(c3)-c3nc2nn3C)c1)C(=O)O]

Methyl (2R)-3-[3-[21-(benzenesulfonyl)-9,9,24,30-tetrafluoro-3,6-dimethyl-)1-oxa-26-thia-3,4,15,21,32,33-hexazahexacyclo[25.3.1.12,5.112,15.017,25.018,22]tritriaconta-1(31),2(33),4,12(32),13,17,19,22,24,27,29-undecaen-6-yl]phenyl]-2-methylpropanoate

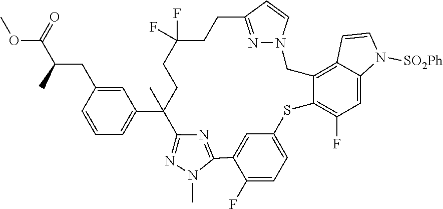

Step A: Exchanging ethyl 3-(3-(6-((1H-pyrazol-3-yl)oxy)-2,5,5-trimethyl-1-(2-methylhydrazineyl)-1-oxohexan-2-yl)phenyl)-2-methylpropanoate (Intermediate 83-5) with methyl (2R)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (Intermediate 97-1, 870 mg, 1.9 mmol), and (6-fluoro-5-(4-fluoro-3-(imino(methylthio)methyl)-phenoxy)-1-(phenylsulfonyl)-1H-indol-4-yl)methyl acetate hydroiodide (Intermediate 82-3) with (6-fluoro-5-((4-fluoro-3-(imino(methylthio)methyl)phenyl)thio)-1-(phenylsulfonyl)-1H-indol-4-yl)methyl acetate (intermediate 82-8, 870 mg, 1.9 mmol), the reaction procedure sequence (Steps A to C) described for Example 99A & 99B, 99A & 99B was used to prepare the title compound (190 mg). Only the major regio-isomer was obtained. MS (ESI): 873 m/z [M+H]⁺, retention time: 1.11 minutes, purity: 98% (214 nm) (LC-MS Method 24).

Methyl (2R)-2-methyl-3-[3-(9,9,24,30-tetrafluoro-3,6-dimethyl-26-oxo-11-oxa-26A4-thia-3,4,15,21,32,33-hexazahexacyclo[25.3.1.12,5.112,15.017,25.018,22]tritriaconta-1(31),2(33),4,12(32),13,17,19,22,24,27,29-undecaen-6-yl)phenyl]propanoate

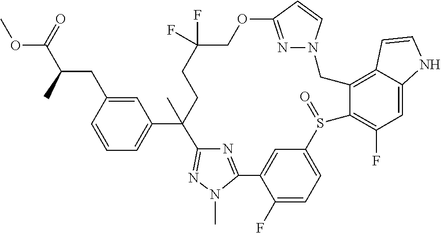 [SMILES: COC(=O)[C@H](C)Cc1cccc(C2(C)CCC(F)(F)COc3ccn(n3)Cc3c(c(F)cc4[nH]ccc34)S(=O)c3ccc(F)c(c3)-c3nc2nn3C)c1]

Step B: To a stirred and cooled (0° C.) solution of Step A product (190 mg, 0.22 mmol) in methanol (10 mL) was added a solution of ammonium molybdate (0.25 g, 0.22 mmol) in hydrogen peroxide (30% in water, 1.2 mL). The mixture was stirred at 0° C. for one hour, then quenched with water (30 mL), and extracted with ethyl acetate (3×30 mL). The combined organic extracts were washed with brine, dried over sodium sulfate, and concentrated. The residue was dissolved in ethanol (10 ml) and potassium carbonate (60 mg, 0.44 mmol). The mixture was stirred at 0° C. for one hour, then diluted with water (40 mL), and extracted with ethyl acetate (3×40 mL). The combined organic extracts were washed with brine, dried over sodium sulfate, and concentrated. The residue was purified by flash chromatography (12 g silica gel column, eluting with 0-90% ethyl acetate in petroleum ether) to give the title compound (100 mg, 65%) as a solid. MS (ESI): 749 m/z [M+H]⁺, retention time: 2.05 minutes, purity: 99% (214 nm) (LC-MS Method 2).

Compounds 124A and 124B: Diastereomers 1 and 2 of (2R)-2-methyl-3-[3-(9,9,24,30-tetrafluoro-3,6-dimethyl-26-oxo-11-oxa-26A4-thia-3,4,15,21,32,33-hexazahexacyclo[25.3.1.12,5.112,15.017,25.018,22]tritriaconta-1(31),2(33),4,12(32),13,17,19,22,24,27,29-undecaen-6-yl)phenyl]propanoic acid

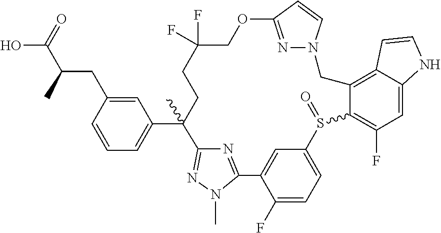 [SMILES: C[C@H](Cc1cccc(C2(C)CCC(F)(F)COc3ccn(n3)Cc3c(c(F)cc4[nH]ccc34)S(=O)c3ccc(F)c(c3)-c3nc2nn3C)c1)C(=O)O]

Step C: The racemic Step B product (100 mg) was subjected to preparative HPLC separation (not chiral). The first eluent, Diastereomer 1 (40 mg, 40%), was further hydrolyzed to Compound 124A (37 mg, 92%) and the second eluent, Diastereomer 2 (38 mg, 38%), was further hydrolyzed to Compound 124B (24 mg, 65%), according to the conditions described for Step E of Example 99A & 99B, 99A & 99B.

Compound 124A: MS (ESI): 735 m/z [M+H]⁺, retention time: 1.75 minutes, purity: >99% (214 nm) (LC-MS Method 26). ¹H NMR (400 MHz, DMSO-d₆) δ 12.1 (s, 1H), 11.66 (s, 1H), 8.28 (s, 1H), 8.07 (s, 1H), 7.78 (t, J=9.6 Hz, 1H), 7.58 (d, J=6.0 Hz, 1H), 7.48 (d, J=2.4 Hz, 1H), 7.25 (d, J=11.3 Hz, 1H), 7.20-7.13 (m, 1H), 7.04 (d, J=13.2 Hz, 1H), 6.99 (d, J=7.2 Hz, 2H), 6.79 (s, 1H), 6.03 (d, J=14.4 Hz, 1H), 5.88 (s, 1H), 5.76 (d, J=14.4 Hz, 1H), 4.21 (m, 1H), 4.10 (m, 1H), 3.60 (s, 3H), 2.89-2.84 (m, 1H), 2.40-2.34 (m, 1H), 2.12-1.96 (m, 3H), 1.68-1.56 (m, 4H), 1.26-1.22 (m, 1H), 0.99-0.94 (m, 3H) ppm.

Compound 124B: MS (ESI): 735 m/z [M+H]⁺, retention time: 1.78 minutes, purity: >99% (214 nm) (LC-MS Method 26). ¹H NMR (400 MHz, DMSO-d₆) δ 12.1 (s, 1H), 11.66 (s, 1H), 8.28 (s, 1H), 8.07 (s, 1H), 7.78 (t, J=9.6 Hz, 1H), 7.58 (d, J=6.0 Hz, 1H), 7.48 (d, J=2.4 Hz, 1H), 7.25 (d, J=11.3 Hz, 1H), 7.20-7.13 (m, 1H), 7.04 (d, J=13.2 Hz, 1H), 6.99 (d, J=7.2 Hz, 2H), 6.79 (s, 1H), 6.03 (d, J=14.4 Hz, 1H), 5.88 (s, 1H), 5.76 (d, J=14.4 Hz, 1H), 4.21 (m, 1H), 4.10 (m, 1H), 3.60 (s, 3H), 2.87 (dd, J=14.1, 8.0 Hz, 1H), 2.37 (t, J=12.8 Hz, 1H), 2.12-1.96 (m, 3H), 1.70-1.56 (m, 4H), 1.27-1.22 (m, 1H), 0.99-0.93 (m, 3H) ppm.

Example 125. Compound 125A, Compound 125B, Compound 125C, and Compound 125C. Diastereomers 1, 2, 3 and 4 of methyl (2S)-2-methyl-3-[3-(9,9,24,30-tetrafluoro-3,6-dimethyl-26-oxo-11-oxa-26λ4-thia-3,4,15,21,32,33-hexazahexacyclo[25.3.1.12,5.112,15.017,25.018,22]-tritriaconta-1(31),2(33),4,12(32),13,17,19,22,24,27,29-undecaen-6-yl)phenyl]propanoate

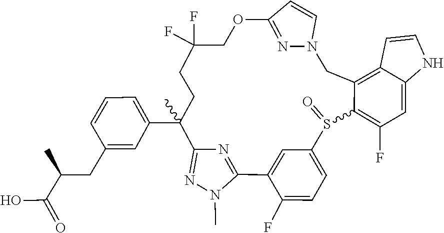 [SMILES: C[C@@H](Cc1cccc(C2(C)CCC(F)(F)COc3ccn(n3)Cc3c(c(F)cc4[nH]ccc34)S(=O)c3ccc(F)c(c3)-c3nc2nn3C)c1)C(=O)O]

Exchanging methyl (2R)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (Intermediate 97-1) with methyl (2S)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (Intermediate 101, 870 mg, 1.9 mmol), the reaction procedure sequence (Steps A to C) described for Example 124A and 124B was used to prepare the title compounds. The 4 diastereomeric methyl ester mixture (210 mg), obtained from corresponding Step B, was first subject to prep-HPLC separation. The first peak is designated as cis/trans Diastereomer A (70 mg); The second peak is designated as cis/trans Diastereomer B (130 mg) (originated from chiral sulfoxide and quaternary chiral carbon center). Both diastereomers were then treated with chiral SFC separation using SFC-80 (Thar, Waters) under the following condition: Column: AD 20*250 mm, 10 μm; Column temperature: 35° C.; Mobile phase: carbon dioxide/isopropanol (0.2% methanol ammonia))=65/35; Flow rate: 80 g/minute; Back pressure: 100 bar; Detection wavelength: 214 nm; Cycle time: 3.6 minutes; Sample solution: 70 mg dissolved in 25 mL methanol for Diastereomer A (130 mg in 25 mL of methanol for Diastereomer B). The first eluent from Diastereomer A, Diastereomer 1 (24 mg, 34%) was further hydrolyzed to Compound 125A (21.8 mg, 91%); The second eluent from Diastereomer A, Diastereomer 2 (34 mg, 99%), was further hydrolyzed to Compound 125B (16 mg, 44%). The first eluent from Diastereomer B, Diastereomer 3 (36 mg, 28%), was hydrolyzed to Compound 125C (19.7 mg, 55%); The second eluent from Diastereomer B, Diastereomer 4 (52 mg), was hydrolyzed to Compound 125D (21.9 mg, 41%).

Compound 125A: MS (ESI): 735 m/z [M+H]⁺, retention time: 1.75 minutes, purity: >99% (214 nm) (LC-MS Method 26). ¹H NMR (400 MHz, DMSO-d₆) δ 12.01 (bs, 1H), 11.66 (s, 1H), 8.29 (s, 1H), 8.10 (s, 1H), 7.78 (t, J=9.6 Hz, 1H), 7.49 (s, 1H), 7.41-7.39 (m, 1H), 7.20 (d, J=11.2 Hz, 1H), 7.10-6.85 (m, 5H), 6.06 (d, J=14.4 Hz, 1H), 5.83 (s, 1H), 5.76 (d, J=14.4 Hz, 1H), 4.27-3.99 (m, 2H), 3.71 (d, J=1.5 Hz, 3H), 2.82-2.75 (m, 1H), 2.27-2.20 (m, 1H), 2.06-1.95 (m, 3H), 1.66-1.50 (m, 4H), 1.24-1.20 (m, 1H), 0.92 (d, J=6.0, 3H) ppm.

Compound 125B: MS (ESI): 735 m/z [M+H]⁺, retention time: 1.78 minutes, purity: >99% (214 nm) (LC-MS Method 26). ¹H NMR (400 MHz, DMSO-d6) δ 12.06 (s, 1H), 11.66 (s, 1H), 8.28 (s, 1H), 8.10 (s, 1H), 7.78 (t, J=9.6 Hz, 1H), 7.48 (d, J=6.0 Hz, 1H), 7.41-7.38 (m, 1H), 7.20 (d, J=11.2 Hz, 1H), 7.09 (t, J=7.7 Hz, 1H), 7.02-6.86 (m, 4H), 6.03 (d, J=14.4 Hz, 1H), 5.83 (s, 1H), 5.76 (d, J=14.4 Hz, 1H), 4.18-4.02 (m, 2H), 3.71 (d, J=1.5 Hz, 3H), 2.82-2.76 (m, 1H), 2.26-2.75 (m, 1H), 2.08-1.96 (m, 3H), 1.60-1.55 (m, 4H), 1.24-1.22 (m, 1H), 0.92 (d, J=6.0 Hz, 3H) ppm.

Compound 125C: MS (ESI): 735 m/z [M+H]⁺, retention time: 1.75 minutes, purity: >99% (214 nm) (LC-MS Method 26). ¹H NMR (400 MHz, DMSO-d₆) δ 11.71 (s, 1H), 8.28 (bs, 1H), 8.08 (s, 1H), 7.78 (t, J=9.6 Hz, 1H), 7.59 (d, J=6.0 Hz, 1H), 7.49 (d, J=2.4 Hz, 1H), 7.26 (d, J=11.2 Hz, 1H), 7.20-7.13 (m, 1H), 7.05-6.96 (m, 3H), 6.78 (s, 1H), 6.03 (d, J=14.4 Hz, 1H), 5.89 (s, 1H), 5.76 (d, J=14.4 Hz, 1H), 4.27-4.06 (m, 2H), 3.61 (s, 3H), 2.89-2.82 (m, 1H), 2.41-2.33 (m, 1H), 2.12-1.96 (m, 3H), 1.66-1.57 (m, 4H), 1.24-1.22 (m, 1H), 0.96-0.93 (m, 3H) ppm.

Compound 125D: MS (ESI): 735 m/z [M+H]⁺, retention time: 1.78 minutes, purity: >99% (214 nm) (LC-MS Method 26). ¹H NMR (400 MHz, DMSO-d6) δ 11.71 (s, 1H), 8.28 (s, 1H), 8.08 (s, 1H), 7.78 (t, J=9.6 Hz, 1H), 7.59 (d, J=6.0 Hz, 1H), 7.49 (d, J=2.4 Hz, 1H), 7.26 (d, J=11.2 Hz, 1H), 7.20-7.13 (m, 1H), 7.07-6.98 (m, 3H), 6.78 (s, 1H), 6.03 (d, J=14.4 Hz, 1H), 5.89 (s, 1H), 5.76 (d, J=14.4 Hz, 1H), 4.25-4.05 (m, 2H), 3.61 (s, 3H), 2.92-2.82 (m, 1H), 2.41-2.33 (m, 1H), 2.12-1.96 (m, 3H), 1.66-1.58 (m, 4H), 1.25-1.19 (m, 1H), 0.97-0.94 (m, 3H) ppm.

Example 126. Compound 126A and Compound 126B: Enantiomers 1 and 2 of 3-[3-(10,16-difluoro-19,22-dimethyl-12-oxa-7,19,20,28,30,32-hexazahexacyclo[25.2.2.113,17.118,21.03,11.04,8]tritriaconta-1(30),3,5,8,10,13,15,17(33),18(32),20,27(31),28-dodecaen-22-yl)phenyl]propanoic acid

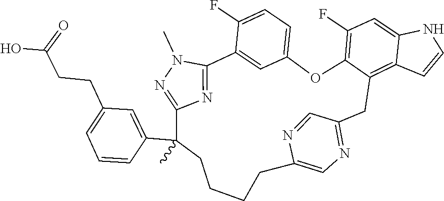 [SMILES: Cn1nc2nc1-c1cc(ccc1F)Oc1c(F)cc3[nH]ccc3c1Cc1cnc(cn1)CCCCC2(C)c1cccc(CCC(=O)O)c1]

4-((5-Allylpyrazin-2-yl)methyl)-5-(3-(3-(2-(3-bromophenyl)hex-5-en-2-yl)-1-methyl-1H-1,2,4-triazol-5-yl)-4-fluorophenoxy)-6-fluoro-1H-indole

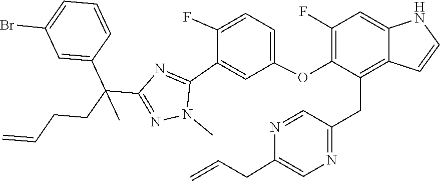 [SMILES: C=CCCC(C)(c1cccc(Br)c1)c1nc(-c2cc(Oc3c(F)cc4[nH]ccc4c3Cc3cnc(CC=C)cn3)ccc2F)n(C)n1]

Step A: To a stirred solution of methyl 5-((4-((5-allylpyrazin-2-yl)methyl)-6-fluoro-1H-indol-5-yl)oxy)-2-fluorobenzimidothioate (Intermediate 82-9, 1.6 g, 2.8 mmol) and 2-(3-bromo phenyl)-N′,2-dimethyl-hex-5-enehydrazide (Intermediate 83-6, 760 mg, 2.44 mmol) in pyridine (20 mL) was added magnesium sulfate (1.6 g, 13.3 mmol). The mixture was stirred at 60° C. for 16 hours, then concentrated. The residue was purified by silica gel column chromatography to afford the title compound (890 mg, 1.28 mmol, 47%). MS (ESI): 695, 697 m/z [M+H]⁺, retention time: 2.15 minutes, purity: 74% (254 nm) (LC-MS Method 28).

(E)-5-(3-(3-(2-(3-Bromophenyl)hex-5-en-2-yl)-1-methyl-1H-1,2,4-triazol-5-yl)-4-fluorophenoxy)-6-fluoro-4-((5-(prop-1-en-1-yl)pyrazin-2-yl)methyl)-1H-indole

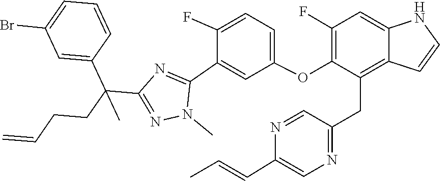 [SMILES: C=CCCC(C)(c1cccc(Br)c1)c1nc(-c2cc(Oc3c(F)cc4[nH]ccc4c3Cc3cnc(/C=C/C)cn3)ccc2F)n(C)n1]

Step B: To a solution of 4-((5-allylpyrazin-2-yl)methyl)-5-(3-(3-(2-(3-bromophenyl)hex-5-en-2-yl)-1-methyl-1H-1,2,4-triazol-5-yl)-4-fluorophenoxy)-6-fluoro-1H-indole (Step A product, 890 mg, 1.28 mmol) in tetrahydrofuran (10 mL) and water (10 mL) was added sodium hydroxide (640 mg, 16.0 mmol). The mixture was stirred at 100° C. for 16 hours, cooled to room temperature and diluted with ethyl acetate (20 mL). The solution was washed with 1N hydrochloride, brine, dried over sodium sulfate, filtered, and concentrated. The residue was purified by flash chromatography to give the title compound (750 mg, 0.99 mmol, 78%) as a yellow oil. MS (ESI): 695, 697 m/z [M+H]⁺, retention time: 2.15 minutes, purity: 74% (254 nm) (LC-MS Method 28).

(25E)-22-(3-Bromophenyl)-10,16-difluoro-19,22-dimethyl-12-oxa-7,19,20,28,30,32-hexazahexacyclo[25.2.2.113,17.118,21.03,11.04,8]tritriaconta-1(30),3,5,8,10,13,15,17(33),18(32),20,25,27(31),28-tridecaene

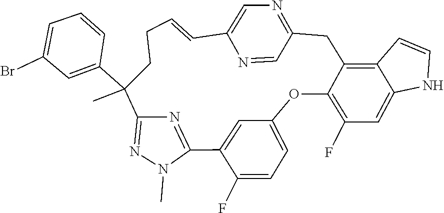 [SMILES: Cn1nc2nc1-c1cc(ccc1F)Oc1c(F)cc3[nH]ccc3c1Cc1cnc(cn1)/C=C/CCC2(C)c1cccc(Br)c1]

Step C: To a stirred solution of (E)-5-(3-(3-(2-(3-bromophenyl)hex-5-en-2-yl)-1-methyl-1H-1,2,4-triazol-5-yl)-4-fluorophenoxy)-6-fluoro-4-((5-(prop-1-en-1-yl)pyrazin-2-yl)methyl)-1H-indole (Step B product, 750 mg, 1.08 mmol) in toluene (800 mL) was added dichloro[1,3-bis(2,4,6-trimethylphenyl)-2-imidazolidinylidene](benzylidene)bis(3-bromopyridine)ruthenium(II) (286 mg, 0.323 mmol). The mixture was stirred at 65° C. for 16 hours and concentrated. The residue was purified by flash chromatography to afford the title compound (500 mg, 0.704 mmol, 65%) as a semi-solid. MS (ESI): 653, 655 m/z [M+H]⁺, retention time: 1.99 minutes, purity: 92% (214 nm) (LC-MS Method 28).

Ethyl (E)-3-[3-[(25E)-10,16-difluoro-19,22-dimethyl-12-oxa-7,19,20,28,30,32-hexazahexacyclo[25.2.2.113,17.118,21.03,11.04,8]tritriaconta-1(30),3,5,8,10,13,15,17(33),18(32),20,25,27(31),28-tridecaen-22-yl]phenyl]prop-2-enoate

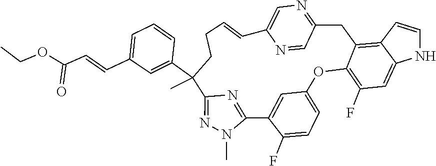 [SMILES: CCOC(=O)/C=C/c1cccc(C2(C)CC/C=C/c3cnc(cn3)Cc3c(c(F)cc4[nH]ccc34)Oc3ccc(F)c(c3)-c3nc2nn3C)c1]

Step D: To a stirred solution of (25E)-22-(3-bromophenyl)-10,16-difluoro-19,22-dimethyl-12-oxa-7,19,20,28,30,32-hexazahexacyclo[25.2.2.113,17.118,21.03,11.04,8]tritriaconta-1(30),3,5,8,10,13,15,17(33),18(32),20,25,27(31),28-tridecaene (Step C product, 385 mg, 0.577 mmol), ethyl (Z)-3-(4,4,5,5-tetramethyl-1,3,2-dioxaborolan-2-yl)prop-2-enoate (261 mg, 1.15 mmol) in toluene (10 mL), ethanol (2 mL) and water (1 mL) was added [1,1′-bis(diphenylphosphino)ferrocene]dichloropalladium(II) (42 mg, 0.0577 mmol) and potassium carbonate (239 mg, 1.73 mmol). The mixture was stirred at 90° C. for 16 hours, cooled to room temperature and diluted with ethyl acetate (10 ml). The solution was washed with water, brine, dried over sodium sulfate, and concentrated. The residue was purified by flash column chromatography eluting with 60% ethyl acetate in hexanes to afford the title compound (300 mg, 0.39 mmol, 68%). LC-MS: MS (ESI): 673 m/z [M+H]⁺, retention time: 1.76 minutes, purity: 88% (214 nm) (LC-MS Method 28).

Ethyl 3-[3-(10,16-difluoro-19,22-dimethyl-12-oxa-7,19,20,28,30,32-hexazahexacyclo[25.2.2.113,17.118,21.03,11.04,8]tritriaconta-1(30),3,5,8,10,13,15,17(33),18(32),20,27(31),28-dodecaen-22-yl)phenyl]propanoate

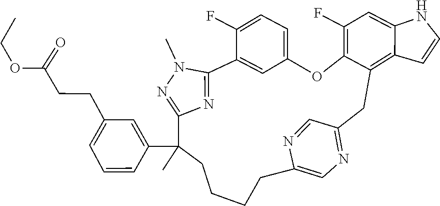 [SMILES: CCOC(=O)CCc1cccc(C2(C)CCCCc3cnc(cn3)Cc3c(c(F)cc4[nH]ccc34)Oc3ccc(F)c(c3)-c3nc2nn3C)c1]

Step E: To a stirred solution of ethyl (E)-3-[3-[(25E)-10,16-difluoro-19,22-dimethyl-12-oxa-7,19,20,28,30,32-hexazahexacyclo[25.2.2.113,17.118,21.03,11.04,8]tritriaconta-1(30),3,5,8,10,13,15,17(33),18(32),20,25,27(31),28-tridecaen-22-yl]phenyl]prop-2-enoate (Step D product, 300 mg, 0.446 mmol) in methanol (8 mL) was added 10% palladium on carbon (wetted with ca. 55% water, 100 mg). The reaction mixture was stirred under hydrogen for 24 hours at room temperature and filtered through a pad of Celite. The filtrate was concentrated to give the title compound (230 mg, 0.272 mmol, 58%) as a yellow liquid. LC-MS: MS (ESI): 677 m/z [M+H]⁺, retention time: 2.11 minutes, purity: 76% (214 nm) (LC-MS Method 28).

Compounds 126A and 126B: Enantiomers 1 and 2 of 3-[3-(10,16-difluoro-19,22-dimethyl-12-oxa-7,19,20,28,30,32-hexazahexacyclo[25.2.2.113,17.118,21.03,11.04,8]tritriaconta-1(30),3,5,8,10,13,15,17(33),18(32),20,27(31),28-dodecaen-22-yl)phenyl]propanoic acid

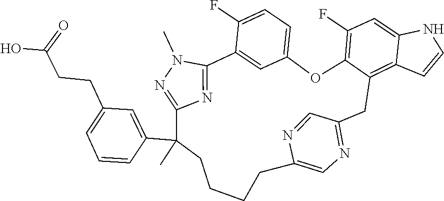 [SMILES: Cn1nc2nc1-c1cc(ccc1F)Oc1c(F)cc3[nH]ccc3c1Cc1cnc(cn1)CCCCC2(C)c1cccc(CCC(=O)O)c1]

Step F: To a solution of ethyl 3-[3-(10,16-difluoro-19,22-dimethyl-12-oxa-7,19,20,28,30,32-hexazahexacyclo[25.2.2.113,17.118,21.03,11.04,8]tritriaconta-1(30),3,5,8,10,13,15,17(33),18(32),20,27(31),28-dodecaen-22-yl)phenyl]propanoate (Step E product, 230 mg, 0.272 mmol) in methanol (1.5 mL), tetrahydrofuran (1.5 mL) and 1N lithium hydroxide in water (2 mL, 2 mmol), the mixture was stirred at room temperature for 5 hours, then acidified with 1.0 M hydrochloric acid to pH ˜5, and extracted with ethyl acetate (3×10 mL). The combined organic phases were washed with brine, dried over anhydrous sodium sulfate, filtered, and concentrated to get the crude title compound (210 mg, 0.2 mmol, 90%). LC-Mass: MS (ESI): 649 m/z [M+H]⁺, retention time: 1.77 minutes, purity: 73% (254 nm) (LC-MS Method 26).

This racemic mixture was subject to chiral SFC separation under the following condition: Instrument: SFC-150 (Waters) (Thar, Waters); Column: AD 20*250 mm, 10 μm; Column temperature: 35° C.; Mobile phase: carbon dioxide/isopropanol (0.5% 7 M methanol Ammonia as additive)=50/50; Flow rate: 100 g/minute; Back pressure: 100 bar; Detection wavelength: 214 nm; Cycle time: 5 minutes; Sample solution: 200 mg dissolved in 30 mL methanol. Injection volume: 2 mL. The first eluent, enantiomer 1 (80 mg, 78% chemical purity), was designated as Compound 126A The second eluent, enantiomer 2 (85 mg, chemical purity: 80%), was designated as Compound 126B (85 mg LCMS: 80%). Further purification by prep-HPLC of those two peaks afford chemical and enantiomerical pure Compound 126A (7.8 mg, 0.012 mmol) and Compound 126B (15.7 mg, 0.024 mmol) as white solids.

Compound 126A: LC-MS: MS (ESI): 649 m/z [M+H]⁺, retention time: 2.04 minutes, purity: >99% (214 nm) (LC-MS Method 22). ¹H NMR (400 MHz, CD₃OD) δ 8.31 (d, J=1.2 Hz, 1H), 8.17 (d, J=1.2 Hz, 1H), 7.51-7.47 (m, 1H), 7.35-7.31 (m, 2H), 7.23 (d, J=10.8 Hz, 1H), 7.17 (t, J=7.6 Hz, 1H), 7.05-7.01 (m, 3H), 6.62 (d, J=2.8 Hz, 1H), 6.25-6.22 (m, 1H), 4.57 (d, J=15.2 Hz, 1H), 4.42 (d, J=15.2 Hz, 1H), 3.60 (s, 3H), 2.94-2.87 (m, 1H), 2.85 (t, J=7.6 Hz, 2H), 2.69-2.62 (m, 1H), 2.52 (t, J=7.6 Hz, 2H), 2.31-2.24 (m, 1H), 1.97-1.90 (m, 1H), 1.89-1.80 (m, 1H), 1.63 (s, 3H), 1.62-1.53 (m, 1H), 1.34-1.29 (m, 1H), 0.73-0.68 (m, 1H) ppm.

Compound 126B: LC-MS: MS (ESI): 649 m/z [M+H]⁺, retention time: 2.04 minutes, purity: >99% (214 nm) (LC-MS Method 22). ¹H NMR (400 MHz, CD₃OD) δ 8.33 (d, J=1.2 Hz, 1H), 8.15 (d, J=1.2 Hz, 1H), 7.48-7.44 (m, 1H), 7.33-7.29 (m, 2H), 7.20 (d, J=10.8 Hz, 1H), 7.14 (t, J=7.6 Hz, 1H), 7.03-6.98 (m, 3H), 6.60 (d, J=2.8 Hz, 1H), 6.23-6.20 (m, 1H), 4.50 (dd, J=57.6, 15.2 Hz, 2H), 3.58 (s, 3H), 2.91-2.86 (m, 1H), 2.82 (t, J=7.6 Hz, 2H), 2.66-2.59 (m, 1H), 2.49 (t, J=7.6 Hz, 2H), 2.30-2.20 (m, 1H), 1.94-1.87 (m, 1H), 1.89-1.80 (m, 1H), 1.60 (s, 3H), 1.62-1.53 (m, 1H), 1.31-1.27 (m, 1H), 0.69-0.66 (m, 1H) ppm.

Example 127. Compound 127A, Compound 127B, Compound 127C, and Compound 127D. Diastereomers 1, 2,3 and 4 of 3-[3-(24,30-difluoro-3,6,11,11-tetramethyl-26-oxo-26λ4-thia-3,4,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]propanoic acid

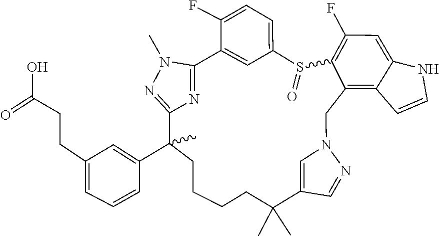 [SMILES: Cn1nc2nc1-c1cc(ccc1F)S(=O)c1c(F)cc3[nH]ccc3c1Cn1cc(cn1)C(C)(C)CCCCC2(C)c1cccc(CCC(=O)O)c1]

(8Z)-21-(benzenesulfonyl)-6-(3-bromophenyl)-24,30-difluoro-3,6,11,11-tetramethyl-26-thia-3,4,14,15,21,33-hexazahexacyclo[25.3.1.12,5.112,15.017,25.018,22]tritriaconta-1(31),2(33),4,8,12(32),13,17,19,22,24,27,29-dodecaene

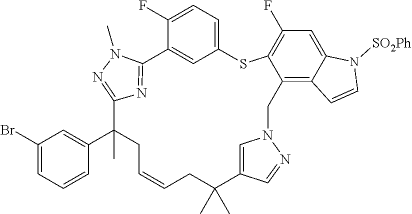

Step A: Exchanging methyl 5-((4-((5-allylpyrazin-2-yl)methyl)-6-fluoro-1H-indol-5-yl)oxy)-2-fluorobenzimidothioate (Intermediate 82-9) with methyl 2-fluoro-5-((6-fluoro-4-((4-(2-methylpent-4-en-2-yl)-1H-pyrazol-1-yl)methyl)-1-(phenylsulfonyl)-1H-indol-5-yl)thio)benzimidothioate hydroiodide (Intermediate 82-10, 2.45 g, 3.2 mmol) and 2-(3-bromo phenyl)-N′,2-dimethyl-hex-5-enehydrazide (Intermediate 83-6) with 2-(3-bromophenyl)-N′,2-dimethylpent-4-enehydrazide (Intermediate 83, 952 mg, 3.2 mmol), the reaction procedure sequence (Steps A and C) described in Example 126A and 126B was followed to afford the title compound (460 mg, 79%) as a solid. LC-MS: MS (ESI): 839, 841 m/z [M+H]⁺, retention time: 2.46 minutes, purity: 98% (254 nm) (LC-MS Method 5).

(8Z)-6-(3-bromophenyl)-24,30-difluoro-3,6,11,11-tetramethyl-26-thia-3,4,14,15,21,33-hexazahexacyclo[25.3.1.12,5.112,15.017,25.018,22]tritriaconta-1(31),2(33),4,8,12(32),13,17,19,22,24,27,29-dodecaene

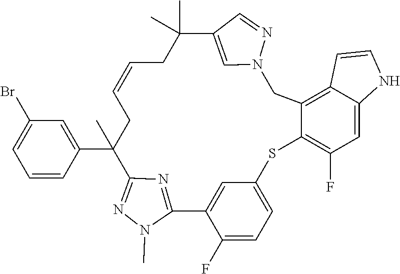 [SMILES: Cn1nc2nc1-c1cc(ccc1F)Sc1c(F)cc3[nH]ccc3c1Cn1cc(cn1)C(C)(C)C/C=C\CC2(C)c1cccc(Br)c1]

Step B: To a solution of Step A product (460 mg, 0.55 mmol) in methanol (10 mL) was added potassium carbonate (151.4 mg, 1.1 mmol). The mixture was stirred at room temperature for 3 hours, then diluted with ethyl acetate (50 mL). The solution was washed with water, brine, dried over magnesium sulfate, filtered, and concentrated. The residue was purified by flash chromatography (12 g silica gel column, eluting with 0-50% ethyl acetate in petroleum) to give the title compound (320 mg, 83.5%) as a solid. LC-MS: MS (ESI): 699, 671 m/z [M+H]⁺, retention time: 2.27 minutes, purity: 90% (254 nm) (LC-MS Method 5).

Ethyl 3-[3-(24,30-difluoro-3,6,11,11-tetramethyl-26-thia-3,4,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]propanoate

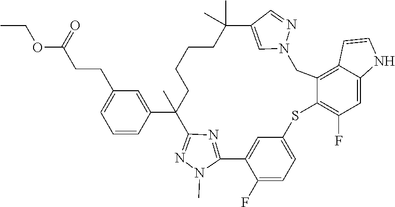 [SMILES: CCOC(=O)CCc1cccc(C2(C)CCCCC(C)(C)c3cnn(c3)Cc3c(c(F)cc4[nH]ccc34)Sc3ccc(F)c(c3)-c3nc2nn3C)c1]

Step C: Exchanging (25E)-22-(3-bromophenyl)-10,16-difluoro-19,22-dimethyl-12-oxa-7,19,20,28,30,32-hexazahexacyclo[25.2.2.113,17.118,21.03,11.04,8]tritriaconta-1(30),3,5,8,10,13,15,17(33),18(32),20,25,27(31),28-tridecaene (Step C product of Example 126A and 126B) with (8Z)-6-(3-bromophenyl)-24,30-difluoro-3,6,11,11-tetramethyl-26-thia-3,4,14,15,21,33-hexazahexacyclo[25.3.1.12,5.112,15.017,25.018,22]tritriaconta-1(31),2(33),4,8,12(32),13,17,19,22,24,27,29-dodecaene (Step B product of this Example, 220 mg, 0.314 mmol), the reaction procedure sequence (Steps D and E) described in Example 126A and 126B was followed to prepare the title compound (52 mg, 23% two steps) as a white solid. LC-MS: MS (ESI): 723 m/z [M+H]⁺, retention time: 2.54 minutes, purity: 94% (254 nm) (LC-MS Method 5).

Ethyl 3-[3-[24,30-difluoro-3,6,11,11-tetramethyl-21-(p-tolylsulfonyl)-26-thia-3,4,14,15,21,33-hexazahexacyclo[25.3.1.12,5.112,15.017,25.018,22]tritriaconta-1(31),2(33),4,12(32),13,17,19,22,24,27,29-undecaen-6-yl]phenyl]propanoate

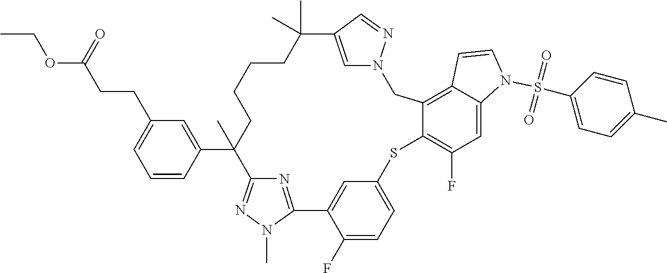 [SMILES: CCOC(=O)CCc1cccc(C2(C)CCCCC(C)(C)c3cnn(c3)Cc3c(c(F)cc4c3ccn4S(=O)(=O)c3ccc(C)cc3)Sc3ccc(F)c(c3)-c3nc2nn3C)c1]

Step D: To a stirred solution of Step C product (160 mg, 0.22 mmol) in acetonitrile (20 mL) was added 1-tosyl-1H-imidazole (98 mg, 0.443 mmol) and 1,8-diazabicyclo[5,4,0]undec-7-ene (0.066 mL, 0.443 mmol) at room temperature. The mixture was stirred at room temperature for 16 hours, diluted with dichloromethane (100 mL). The solution was washed with water, brine, dried over magnesium sulfate, filtered, and concentrated. The residue was purified by silica gel column chromatography (12 g silica gel column, eluting with 0-50% ethyl acetate in petroleum ether) to give the title compound (169 mg, 87%) as a solid. LC-MS: MS (ESI): 877 m/z [M+H]⁺, retention time: 2.50 minutes, purity: 84% (254 nm) (LC-MS Method 5).

Diastereomer A (a Mixture of Two Diastereomers) and Diastereomer B (a Mixture of Two Diastereomers) of Ethyl 3-[3-[24,30-difluoro-3,6,11,11-tetramethyl-26-oxo-21-(p-tolylsulfonyl)-26A4-thia-3,4,14,15,21,33-hexazahexacyclo[25.3.1.12,5.112,15.017,25.018,22]tritriaconta-1(31),2(33),4,12(32),13,17,19,22,24,27,29-undecaen-6-yl]phenyl]propanoate

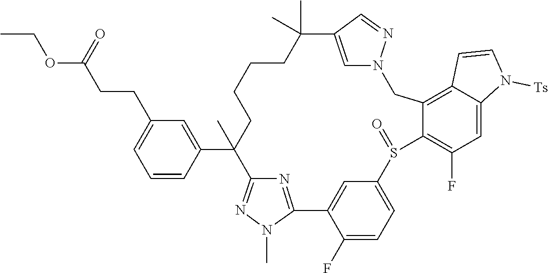 [SMILES: CCOC(=O)CCc1cccc(C2(C)CCCCC(C)(C)c3cnn(c3)Cc3c(c(F)cc4c3ccn4S(=O)(=O)c3ccc(C)cc3)S(=O)c3ccc(F)c(c3)-c3nc2nn3C)c1]

Step E: To a stirred solution of Step D product (169 mg, 0.19 mmol) in acetonitrile (40 mL) was added (R,R)-diethyl tartrate (0.33 mL, 1.93 mmol), titanium tetraisopropanolate (0.29 mL, 0.963 mmol) and tert-butyl hydroperoxide (0.093 mL, 0.963 mmol) at room temperature. The mixture was stirred at 40° C. for 16 hours, then diluted with ethyl acetate (100 mL). The solution was washed with water, brine, dried over magnesium sulfate, filtered, and concentrated. The residue was purified by automated silica gel column chromatography (12 g silica gel column, eluting with 0-100% ethyl acetate in petroleum ether) to give the title compound (125 mg, 73% diastereomers of four) as a solid.

This diastereomeric mixture was subject to chiral SFC separation under the following condition: Instrument: SFC-150 (Waters) (Thar, Waters); Column: AD 20*250 mm, 10 μm; Column temperature: 35° C.; Mobile phase: carbon dioxide/isopropanol (0.2% 7 M methanol Ammonia as additive)=60/40; Flow rate: 100 g/minute; Back pressure: 100 bar; Detection wavelength: 214 nm; Cycle time: 6.79 minutes; Sample solution: 125 mg dissolved in 15 mL methanol. Injection volume: 1.5 mL. The first eluent (39 mg, 31%, mixture of two diastereomers) was designated as Diastereomer A. The second eluent (40 mg, 32%, mixture of two diastereomers) was designated as Diastereomer B.

Diastereomer A: LC-MS: MS (ESI): 893 m/z [M+H]⁺, retention time: 2.22 minutes, purity: 94% (254 nm) (LC-MS Method 5).

Diastereomer B: LC-MS: MS (ESI): 893 m/z [M+H]⁺, retention time: 2.22 minutes, purity: >99% (254 nm) (LC-MS Method 5).

Compounds 127A and 127B: Diastereomers 1 and 2 of 3-[3-(24,30-difluoro-3,6,11,11-tetramethyl-26-oxo-26A4-thia-3,4,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]propanoic acid

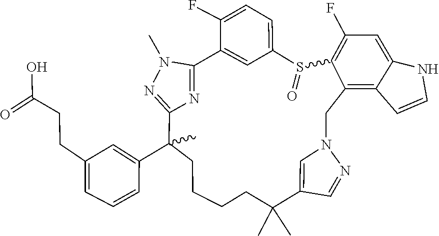 [SMILES: Cn1nc2nc1-c1cc(ccc1F)S(=O)c1c(F)cc3[nH]ccc3c1Cn1cc(cn1)C(C)(C)CCCCC2(C)c1cccc(CCC(=O)O)c1]

Step F: To a stirred solution of Diastereomer A of Step E of this example (39 mg, 0.0437 mmol) in tetrahydrofuran (1 mL) was added lithium hydroxide monohydrate (0.218 mL, 1M in water). The reaction mixture was stirred at room temperature for 48 hours and then acidified with 1M hydrochloric acid to pH ˜4. The solution was extracted with ethyl acetate (2×15 mL). The combined organic extracts were washed with brine, dried over sodium sulfate, filtered and concentrated. The residue was purified by prep-HPLC to give the first eluent (7.4 mg, 24%), designated as Compound 127A, Diastereomer 1, and second eluent (6.2 mg, 20.0%), designated as Compound 127B, Diastereomer 2, both as white solid.

Compound 127A, Diastereomer 1: LC-MS: MS (ESI): 711 m/z [M+H]⁺, retention time: 1.82 minutes, purity: >99% (254 nm) (LC-MS Method 26). ¹H NMR (400 MHz, CD₃OD) δ 8.37-8.30 (m, 1H), 7.63 (t, J=9.2 Hz, 1H), 7.40 (d, J=3.2 Hz, 1H), 7.34 (s, 1H), 7.23-7.21 (m, 2H), 7.15 (t, J=7.6 Hz, 1H), 7.09 (s, 1H), 7.05-7.00 (m, 3H), 6.59 (brs, 1H), 6.25-6.21 (m, 1H), 5.89-5.84 (m, 1H), 3.65 (s, 3H), 2.84 (t, J=7.6 Hz, 2H), 2.52 (t, J=7.6 Hz, 2H), 2.27-2.22 (m, 1H), 2.08-2.03 (m, 1H), 1.58 (s, 3H), 1.39-1.27 (m, 4H), 1.12 (s, 3H), 1.05 (s, 3H), 1.02-0.98 (m, 1H), 0.91-0.87 (m, 1H) ppm.

Compound 127B, Diastereomer 2: LC-MS: MS (ESI): 711 m/z [M+H]⁺, retention time: 1.87 minutes, purity: 99% (254 nm) (LC-MS Method 26). ¹H NMR (400 MHz, CD₃OD) δ 8.41-8.35 (m, 1H), 7.66-7.60 (m, 3H), 7.45 (d, J=3.2 Hz, 1H), 7.16-7.11 (m, 2H), 7.07-7.01 (m, 2H), 6.96-6.87 (m, 3H), 6.22 (d, J=14.8 Hz, 1H), 5.84 (d, J=14.8 Hz, 1H), 3.83 (d, J=1.6 Hz, 3H), 2.77 (t, J=7.6 Hz, 2H), 2.45 (t, J=7.6 Hz, 2H), 2.01-1.96 (m, 2H), 1.60 (s, 3H), 1.51-1.32 (m, 4H), 1.13 (s, 3H), 1.10 (s, 3H), 0.82-0.72 (m, 1H), 0.68-0.60 (m, 1H) ppm.

Compounds 127C and 127D, Diastereomers 3 and 4 of 3-[3-(24,30-difluoro-3,6,11,11-tetramethyl-26-oxo-26A4-thia-3,4,14,15,21,33-hexazahexacyclo[25.3.1.12,5.112,15.017,25.018,22]tritriaconta-1(31),2(33),4,12(32),13,17,19,22,24,27,29-undecaen-6-yl)phenyl]propanoic acid

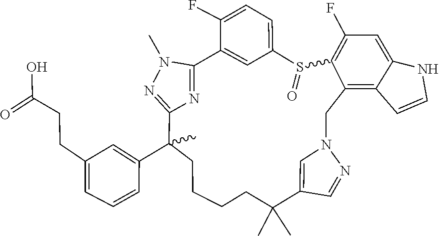 [SMILES: Cn1nc2nc1-c1cc(ccc1F)S(=O)c1c(F)cc3[nH]ccc3c1Cn1cc(cn1)C(C)(C)CCCCC2(C)c1cccc(CCC(=O)O)c1]

Step G: Utilizing the same procedure as described in Step F, Diastereomer B of Step D product was hydrolyzed and further purified by prep-HPLC to afford the first eluent (8.0 mg, 25%), designated as Compound 127C, Diastereomer 3; and the second eluent (9.3 mg, 29%), designated as Compound 127D, Diastereomer 4, both as white solid.

Compound 127C: LC-MS: MS (ESI): 711 m/z [M+H]⁺, retention time: 1.82 minutes, purity: >99% (254 nm) (LC-MS Method 26). ¹H NMR (400 MHz, CD₃OD) δ 8.37-8.31 (m, 1H), 7.62 (t, J=9.2 Hz, 1H), 7.40 (d, J=3.2 Hz, 1H), 7.34 (s, 1H), 7.25-7.21 (m, 2H), 7.15 (t, J=7.6 Hz, 1H), 7.09 (s, 1H), 7.05-7.00 (m, 3H), 6.59 (br s, 1H), 6.22 (d, J=14.8 Hz, 1H), 5.86 (d, J=14.8 Hz, 1H), 3.65 (s, 3H), 2.84 (t, J=8.0 Hz, 2H), 2.52 (t, J=8.0 Hz, 2H), 2.29-2.21 (m, 1H), 2.08-2.01 (m, 1H), 1.58 (s, 3H), 1.42-1.25 (m, 4H), 1.12 (s, 3H), 1.05 (s, 3H), 1.02-0.98 (m, 1H), 0.92-0.85 (m, 1H) ppm.

Compound 127D, Diastereomer 4: LC-MS: MS (ESI): 711 m/z [M+H]⁺, retention time: 1.87 minutes, purity: 99% (254 nm) (LC-MS Method 26). ¹H NMR (400 MHz, CD₃OD) δ 8.41-8.35 (m, 1H), 7.66-7.61 (m, 3H), 7.45 (d, J=3.6 Hz, 1H), 7.15-7.11 (m, 2H), 7.07-7.01 (m, 2H), 6.96-6.87 (m, 3H), 6.22 (d, J=14.8 Hz, 1H), 5.84 (d, J=14.8 Hz, 1H), 3.83 (d, J=1.6 Hz, 3H), 2.77 (t, J=7.6 Hz, 2H), 2.45 (t, J=7.6 Hz, 2H), 1.98 (t, J=6.8 Hz, 2H), 1.60 (s, 3H), 1.50-1.35 (m, 4H), 1.11 (d, J=12.8 Hz, 6H), 0.79-0.74 (m, 1H), 0.68-0.59 (m, 1H) ppm.

Example 128. Compound 128. 24,30-Difluoro-3,6,11,11-tetramethyl-6-phenyl-26λ4-thia-3,4,14,15,21,33-hexazahexacyclo[25.3.1.12,5.112,15.017,25.018,22]tritriaconta-1(31),2(33),4,12(32),13,17,19,22,24,27,29-undecaene 26-oxide

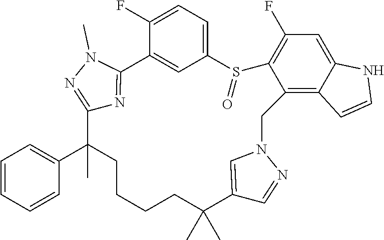 [SMILES: Cn1nc2nc1-c1cc(ccc1F)S(=O)c1c(F)cc3[nH]ccc3c1Cn1cc(cn1)C(C)(C)CCCCC2(C)c1ccccc1]

24,30-difluoro-3,6,11,11-tetramethyl-6-phenyl-26-thia-3,4,14,15,21,33-hexazahexacyclo[25.3.1.12,5.112,15.017,25.018,22]tritriaconta-1(31),2(33),4,12(32),13,17,19,22,24,27,29-undecaene

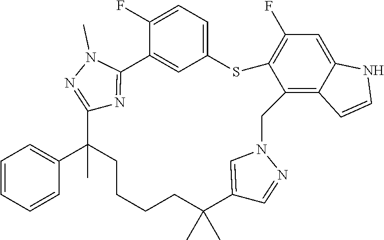 [SMILES: Cn1nc2nc1-c1cc(ccc1F)Sc1c(F)cc3[nH]ccc3c1Cn1cc(cn1)C(C)(C)CCCCC2(C)c1ccccc1]

Step A: To a stirred solution of (8Z)-6-(3-bromophenyl)-24,30-difluoro-3,6,11,11-tetramethyl-26-thia-3,4,14,15,21,33-hexazahexacyclo[25.3.1.12,5.112,15.017,25.018,22]tritriaconta-1(31),2(33),4,8,12(32),13,17,19,22,24,27,29-dodecaene (140 mg, 0.20 mmol) (Step B product of Examples 127A, 127B, 127C, 127D) in ethanol (10 mL) was added palladium on carbon (50 mg, 50% wet, 10%). The reaction mixture was stirred under hydrogen for 2 hours at 50° C., then filtered through a pad of Celite and washed with ethanol (30 mL). The filtrate was concentrated. To the stirred solution of the residue in ethanol (10 mL) was added platinum dioxide (48.7 mg, 0.21 mmol). The reaction was stirred under hydrogen for another 6 hours at 50° C. for complete hydrogenation. The mixture was filtered through a pad of Celite and washed with ethanol (30 mL). The filtrate was concentrated. The residue was purified by prep-HPLC to give the title compound (61 mg, 46%) as a white solid. LC-MS: MS (ESI): 623 m/z [M+H]⁺, retention time: 2.24 minutes, purity: 95% (254 nm) (LC-MS Method 5).

24,30-Difluoro-3,6,11,11-tetramethyl-6-phenyl-21-(p-tolylsulfonyl)-26-thia-3,4,14,15,21,33-hexazahexacyclo[25.3.1.12,5.112,15.017,25.018,22]tritriaconta-1(31),2(33),4,12(32),13,17,19,22,24,27,29-undecaene

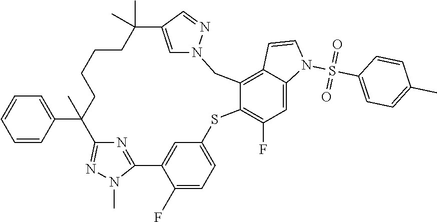 [SMILES: Cc1ccc(S(=O)(=O)n2ccc3c4c(c(F)cc32)Sc2ccc(F)c(c2)-c2nc(nn2C)C(C)(c2ccccc2)CCCCC(C)(C)c2cnn(c2)C4)cc1]

Step B: To a stirred solution of Step A product (61 mg, 0.098 mmol) in acetonitrile (5 mL) was added 1-tosyl-1H-imidazole (43.5 mg, 0.196 mmol) and 1,8-diazabicyclo[5,4,0]undec-7-ene (29.8 mg, 0.196 mmol) at room temperature. The reaction mixture was stirred at room temperature for 16 hours, then diluted with dichloromethane (30 mL). The solution was washed with water, dried over magnesium sulfate, filtered and concentrated. The residue was purified by silica gel column chromatography (12 g silica gel column, eluting with 0-50% ethyl acetate in petroleum ether) to give the title compound (65 mg, 85%) as an oil. LC-MS: MS (ESI): 777 m/z [M+H]⁺, retention time: 2.53 minutes, purity: 84% (254 nm) (LC-MS Method 5).

24,30-Difluoro-3,6,11,11-tetramethyl-6-phenyl-21-(p-tolylsulfonyl)-26A4-thia-3,4,14,15,21,33-hexazahexacyclo[25.3.1.12,5.112,15.017,25.018,22]tritriaconta-1(31),2(33),4,12(32),13,17,19,22,24,27,29-undecaene 26-oxide

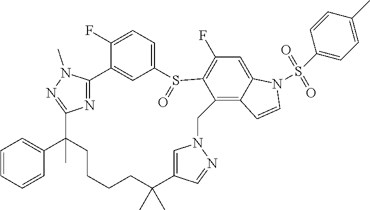 [SMILES: Cc1ccc(S(=O)(=O)n2ccc3c4c(c(F)cc32)S(=O)c2ccc(F)c(c2)-c2nc(nn2C)C(C)(c2ccccc2)CCCCC(C)(C)c2cnn(c2)C4)cc1]

Step C: To a stirred solution of Step B product (65 mg, 0.084 mmol) in acetonitrile (5 mL) was added (R,R)-diethyl tartrate (0.14 mL, 0.837 mmol), titanium tetraisopropanolate (5.00 eq, 0.12 mL, 0.418 mmol) and tert-butyl hydroperoxide (0.041 mL, 0.42 mmol) at room temperature. The reaction mixture was stirring at 40° C. for 16 hours. The reaction mixture was diluted with ethyl acetate, washed with water, dried over magnesium sulfate, filtered, and concentrated. The residue was purified by silica gel column chromatography (12 g silica gel column, eluting with 0-100% ethyl acetate in petroleum ether) to give the title compound (35 mg, 53%) as a white solid. LC-MS: MS (ESI): 793 m/z [M+H]⁺, retention time: 2.27 minutes, purity: 85% (254 nm) (LC-MS Method 5).

Compound 128. 24,30-Difluoro-3,6,11,11-tetramethyl-6-phenyl-26A4-thia-3,4,14,15,21,33-hexazahexacyclo[25.3.1.12,5.112,15.017,25.018,22]tritriaconta-1(31),2(33),4,12(32),13,17,19,22,24,27,29-undecaene 26-oxide

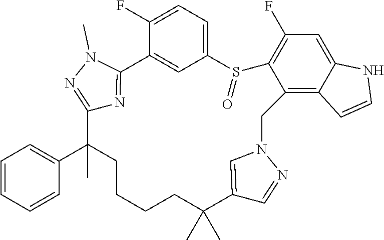 [SMILES: Cn1nc2nc1-c1cc(ccc1F)S(=O)c1c(F)cc3[nH]ccc3c1Cn1cc(cn1)C(C)(C)CCCCC2(C)c1ccccc1]

Step D: To a stirred solution of Step C product (34 mg, 0.043 mmol) in methanol (5 mL) was added potassium carbonate (11.8 mg, 0.086 mmol). The reaction was stirred at room temperature for 2 hours, then diluted with ethyl acetate (30 mL). The solution was washed with brine, dried over sodium sulfate, filtered, and concentrated. The residue was purified by prep-HPLC to give the title compound (10.3 mg, 38%, mixture of four diastereomers) as a white solid.

LC-MS: MS (ESI): 639 m/z [M+H]⁺, retention time: 2.03 minutes, purity: 100% (254 nm) (LC-MS Method 5). ¹H NMR (400 MHz, CD₃OD) δ 11.28 (s, 1H), 8.40-8.32 (m, 1H), 7.67-7.62 (m, 2H), 7.46-7.40 (m, 1H), 7.37-7.32 (m, 0.5H), 7.24-7.20 (m, 3H), 7.15-7.01 (m, 5H), 6.58 (brs, 0.5H), 6.28-6.20 (m, 1H), 5.89-5.80 (m, 1H), 3.84 (d, J=1.2 Hz, 1.5H), 3.66 (d, J=0.8 Hz, 1.5H), 2.30-0.23 (m, 0.5H), 2.09-2.03 (m, 0.5H), 1.99 (d, J=4.0 Hz, 1H), 1.61-1.59 (m, 3H), 1.50-1.29 (m, 4H), 1.13-1.12 (m, 3H), 1.05 (s, 3H), 1.02-0.98 (m, 0.5H), 0.93-0.85 (m, 0.5H), 0.81-0.71 (m, 0.5H), 0.69-0.61 (m, 0.5H) ppm.

Example 129. Compound 129A and Compound 129B: Diastereomers 1 and 2 of 3-[3-(24,30-difluoro-11,11-dimethyl-26-oxo-9-oxa-26λ4-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl)phenyl]propanoic acid

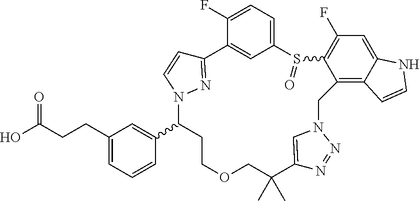 [SMILES: CC1(C)COCCC(c2cccc(CCC(=O)O)c2)n2ccc(n2)-c2cc(ccc2F)S(=O)c2c(F)cc3[nH]ccc3c2Cn2cc1nn2]

(5-((3-(1-(1-(3-Bromophenyl)-3-((2,2-dimethylbut-3-yn-1-yl)oxy)propyl)-1H-pyrazol-3-yl)-4-fluorophenyl)thio)-6-fluoro-1-tosyl-1H-indol-4-yl)methanol

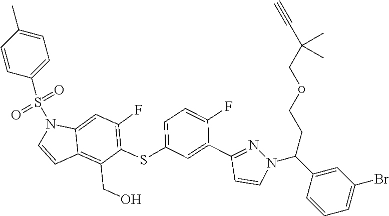 [SMILES: C#CC(C)(C)COCCC(c1cccc(Br)c1)n1ccc(-c2cc(Sc3c(F)cc4c(ccn4S(=O)(=O)c4ccc(C)cc4)c3CO)ccc2F)n1]

Step A: To a stirred solution of (6-fluoro-5-((4-fluoro-3-(1H-pyrazol-3-yl)phenyl)thio)-1-tosyl-1H-indol-4-yl)methanol (Intermediate 55-5, 720 mg, 1.41 mmol) and 1-bromo-3-(1-bromo-3-((2,2-dimethylbut-3-yn-1-yl)oxy)propyl)benzene (Intermediate 98, 632 mg, 1.69 mmol) in N,N-dimethylformamide (10 mL) was added cesium carbonate (3.00 eq, 1376 mg, 4.22 mmol). The mixture was stirred at room temperature for 8 hours, quenched with water, and extracted with ethyl acetate (2×30 mL). The combined organic phase was washed with brine, dried over sodium sulfate, filtered, and concentrated. The residue was purified by flash chromatography to afford the title compound (270 mg, 0.34 mmol, 24% yield). LC-MS: MS (ESI): 804 m/z [M+H]⁺, retention time: 2.70 minutes, purity: 92% (254 nm) (LC-MS Method 28).

4-(Azidomethyl)-5-((3-(1-(1-(3-bromophenyl)-3-((2,2-dimethylbut-3-yn-1-yl)oxy)propyl)-1H-pyrazol-3-yl)-4-fluorophenyl)thio)-6-fluoro-1-tosyl-1H-indole

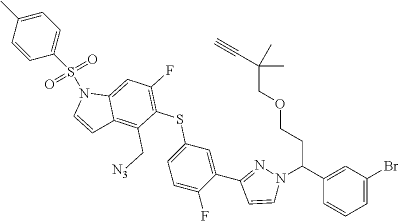 [SMILES: C#CC(C)(C)COCCC(c1cccc(Br)c1)n1ccc(-c2cc(Sc3c(F)cc4c(ccn4S(=O)(=O)c4ccc(C)cc4)c3CN=[N+]=[N-])ccc2F)n1]

Step B: To a stirred solution of Step A product (270 mg, 0.336 mmol) in tetrahydrofuran (5 mL) was added 1,8-diazabicyclo[5.4.0]undec-7-ene (102 mg, 0.67 mmol) and phosphorazidic acid, diphenylester (185 mg, 0.671 mmol). The mixture was stirred at 50° C. for 4 hours, quenched with saturated ammonium chloride (5 mL), and extracted with ethyl acetate (3×4 mL). The combined organic phase was washed with water, brine, dried over sodium sulfate, filtered, and concentrated. The residue was purified by flash chromatography to give the title compound (180 mg, 0.22 mmol, 65%). LC-MS: MS (ESI): 831, 829 m/z [M+H]⁺, retention time: 2.50 minutes, purity: 92% (254 nm) (LC-MS Method 17).

6-(3-Bromophenyl)-24,30-difluoro-11,11-dimethyl-21-(p-tolylsulfonyl)-9-oxa-26-thia-5,13,14,15,21,33-hexazahexacyclo[25.3.1.12,5.112,15.017,25.018,22]tritriaconta-1(31),2(33),3,12(32),13,17,19,22,24,27,29-undecaene

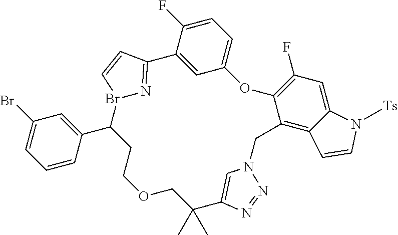

Step C: To a stirred solution of Step B product (180 mg, 0.217 mmol) in dichloromethane (5 mL) was added tetrakis(acetonitrile)copper(I) tetrafluoroborate (0.100 eq, 6.8 mg, 0.0217 mmol) and tris[(1-benzyl-1H-1,2,3-triazol-4-yl)methyl]amine (0.100 eq, 12 mg, 0.0217 mmol). The mixture was stirred at 45° C. for 6 hours and then concentrated. The residue was purified by flash chromatography to afford the title compound (110 mg, 0.13 mmol, 61%) as a yellow oil. LC-MS: MS (ESI): 831, 829 m/z [M+H]⁺, retention time: 2.33 minutes, purity: 83% (254 nm) (LC-MS Method 17).

Ethyl (E)-3-[3-[24,30-difluoro-11,11-dimethyl-21-(p-tolylsulfonyl)-9-oxa-26-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl]phenyl]prop-2-enoate

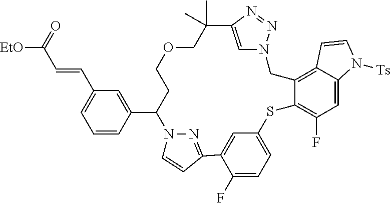 [SMILES: CCOC(=O)/C=C/c1cccc(C2CCOCC(C)(C)c3cn(nn3)Cc3c(c(F)cc4c3ccn4S(=O)(=O)c3ccc(C)cc3)Sc3ccc(F)c(c3)-c3ccn2n3)c1]

Step D. To a stirred solution of Step C product (110 mg, 0.133 mmol) and ethyl (Z)-3-(4,4,5,5-tetramethyl-1,3,2-dioxaborolan-2-yl)prop-2-enoate (60 mg, 0.265 mmol) in toluene (10 mL), ethanol (2 mL) and water (1 mL) were added [1,1′-Bis(diphenylphosphino) ferrocene]dichloropalladium (II) (9.7 mg, 0.0133 mmol) and potassium carbonate (55 mg, 0.398 mmol). The mixture was stirred at 90° C. for 16 hours, cooled to room temperature, and diluted with ethyl acetate (20 mL). The solution was washed with water, brine, dried over magnesium sulfate, and concentrated. The residue was purified by flash column chromatography (eluting with 60% ethyl acetate in hexane) to afford the title compound (90 mg, 0.10 mmol, 74%) as a yellow solid. LC-MS: MS (ESI): 849 m/z [M+H]⁺, retention time: 2.28 minutes, purity: 92% (254 nm) (LC-MS Method 28).

Ethyl 3-[3-[24,30-difluoro-11,11-dimethyl-21-(p-tolylsulfonyl)-9-oxa-26-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl]phenyl]propanoate

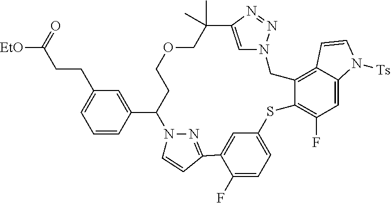 [SMILES: CCOC(=O)CCc1cccc(C2CCOCC(C)(C)c3cn(nn3)Cc3c(c(F)cc4c3ccn4S(=O)(=O)c3ccc(C)cc3)Sc3ccc(F)c(c3)-c3ccn2n3)c1]

Step E: To a stirred solution of Step D product (90 mg, 0.106 mmol) in ethanol (4 mL) was added 10% palladium on carbon (wetted with ca. 55% Water, 30 mg). The reaction mixture was stirred under hydrogen for 6 hours at room temperature, then filtered through a pad of Celite and washed with ethanol. The filtrate was concentrated to give the title compound (80 mg, 0.094 mmol, 88%) as yellow oil. LC-MS: MS (ESI): 851 m/z [M+H]⁺, retention time: 2.68 minutes, purity: 68% (254 nm) (LC-MS Method 28).

Diastereomers 1 and 2 of Ethyl 3-[3-[24,30-difluoro-11,11-dimethyl-26-oxo-21-(p-tolylsulfonyl)-9-oxa-26A4-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl]phenyl]propanoate

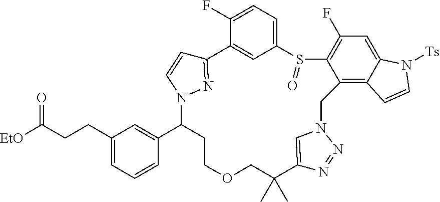 [SMILES: CCOC(=O)CCc1cccc(C2CCOCC(C)(C)c3cn(nn3)Cc3c(c(F)cc4c3ccn4S(=O)(=O)c3ccc(C)cc3)S(=O)c3ccc(F)c(c3)-c3ccn2n3)c1]

Step F: To a stirred solution of Step E product (80 mg, 0.094 mmol) in acetonitrile (5 mL) was added titanium(IV) tetraisopropoxide (134 mg, 0.470 mmol), (R,R)-hydrobenzoin (10.0 eq, 201 mg, 0.940 mmol) and tert-butyl hydroperoxide (93 mg, 1.03 mmol). The mixture was stirred at 60° C. for 6 hours, then diluted with water and extracted with ethyl acetate (3×30 mL). The combined organic phase was washed with brine, dried over sodium sulfate, filtered and concentrated to afford the crude title compound (150 mg, 0.17 mmol, Purity: 35%) as yellow oil. LC-MS: MS (ESI): 867 m/z [M+H]⁺, retention time: 2.07 minutes, purity: 35% (254 nm) (LC-MS Method 26).

The above crude product (150 mg, 0.17 mmol, Purity: 35%) was purified by prep-HPLC. The first eluent was designated as Diastereomer 1 (13 mg, 8.7%, a mixture of two enantiomers); The second eluent was designated as Diastereomer 2 (12 mg, 8%, a mixture of two enantiomers).

Both diastereomers are white solids.

Diastereomer 1: LC-MS: MS (ESI): 867 m/z [M+H]⁺, retention time: 2.05 minutes, purity: >99% (214 nm) (LC-MS Method 26).

Diastereomer 2: LC-MS: MS (ESI): 867 m/z [M+H]⁺, retention time: 2.05 minutes, purity: >99% (214 nm) (LC-MS Method 26).

Compound 129A: Diastereomers 1 of 3-[3-(24,30-difluoro-11,11-dimethyl-26-oxo-9-oxa-26A4-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl)phenyl]propanoic acid

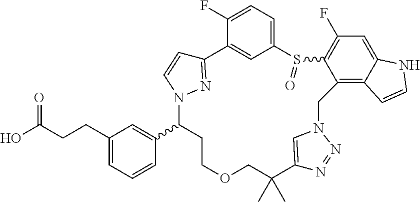 [SMILES: CC1(C)COCCC(c2cccc(CCC(=O)O)c2)n2ccc(n2)-c2cc(ccc2F)S(=O)c2c(F)cc3[nH]ccc3c2Cn2cc1nn2]

Step G: To a stirred solution of Step F product (13 mg, 0.015 mmol) in tetrahydrofuran (2 mL) and water (2 mL) was added lithium hydroxide monohydrate (44 mg, 1.05 mmol). The reaction was stirred at 30° C. for 8 hours, then diluted with water (2 mL), acidified with 1N hydrochloric acid to pH ˜4, and extracted with ethyl acetate (3×4 mL). The combined organic layer was washed with brine, dried over sodium sulfate, filtered, and concentrated. The residue was purified by prep-HPLC to give the title compound (3.4 mg, 0.005 mmol, 16%, mixture of 2 enantiomers) as a white solid.

Compound 129A: LC-MS: MS (ESI): 685 m/z [M+H]⁺, retention time: 1.91 minutes, purity: >99% (214 nm) (LC-MS Method 26). ¹H NMR (400 MHz, CD₃OD) δ 7.92 (s, 1H), 7.72 (s, 1H), 7.54 (d, J=2.1 Hz, 2H), 7.49-7.40 (m, 3H), 7.21-7.01 (m, 2H), 7.03-6.87 (m, 2H), 6.81 (s, 1H), 6.59-6.52 (m, 2H), 6.28-6.24 (m, 1H), 5.29-5.25 (m, 1H), 3.39-.35 (m, 1H), 3.13-2.99 (m, 1H), 2.83 (t, J=7.8 Hz, 2H), 2.50 (t, J=7.8 Hz, 2H), 2.36-2.31 (m, 1H), 1.93-1.90 (s, 1H), 1.38-1.25 (m, 2H), 1.21 (s, 3H), 1.02 (s, 3H).

Compound 129B: Diastereomers 2 of 3-[3-(24,30-difluoro-11,11-dimethyl-26-oxo-9-oxa-26A4-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl)phenyl]propanoic acid

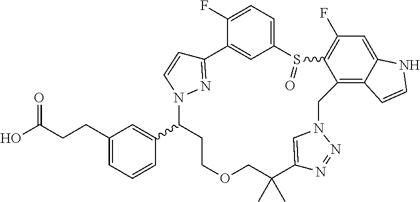 [SMILES: CC1(C)COCCC(c2cccc(CCC(=O)O)c2)n2ccc(n2)-c2cc(ccc2F)S(=O)c2c(F)cc3[nH]ccc3c2Cn2cc1nn2]

Step H. Utilizing the same procedures described in Step G, The diastereomer 2 of Step F product was deprotected, hydrolyzed, and purified by prep-HPLC to give the title compound (3.4 mg, 0.0047 mmol, 15%, mixture of two enantiomers) as a white solid.

Compound 129B: LC-MS: MS (ESI): 685 m/z [M+H]⁺, retention time: 1.93 minutes, purity: >99% (214 nm) (LC-MS Method 26). ¹H NMR (400 MHz, CD₃OD) δ 7.94 (s, 1H), 7.72-7.60 (m, 2H), 7.49-7.27 (m, 4H), 7.18 (t, J=7.6 Hz, 1H), 7.11 (d, J=7.6 Hz, 1H), 7.04 (s, 1H), 6.99 (d, J=7.6 Hz, 1H), 6.65-6.62 (m, 2H), 6.35-6.20 (m, 2H), 5.40-5.30 (m, 1H), 3.50-3.40 (m, 1H), 3.28-3.20 (m, 1H), 3.17-3.05 (m, 1H), 2.85 (t, J=7.8 Hz, 2H), 2.49 (t, J=7.8 Hz, 2H), 2.42-2.30 (m, 1H), 1.35-1.25 (m, 3H), 1.20-1.10 (m, 5H) ppm.

Example 130. Compound 130A and Compound 130B. Diastereomers 1 and 2 of 3-[3-(24,30-difluoro-11,11-dimethyl-26-oxo-26λ4-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl)phenyl]propanoic acid

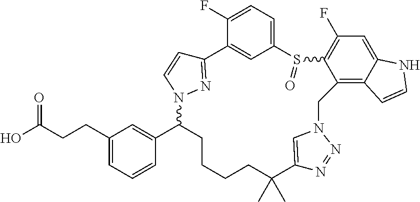 [SMILES: CC1(C)CCCCC(c2cccc(CCC(=O)O)c2)n2ccc(n2)-c2cc(ccc2F)S(=O)c2c(F)cc3[nH]ccc3c2Cn2cc1nn2]

Exchanging 1-bromo-3-(1-bromo-3-((2,2-dimethylbut-3-yn-1-yl)oxy)propyl)benzene (Intermediate 98) with 1-bromo-3-(1-bromo-6,6-dimethyloct-7-yn-1-yl)benzene (Intermediate 112, 764 mg, 1.4 mmol), the reaction procedure sequence (steps A to G) described for Example 129A and 129B was used to prepare the title compounds. The first eluent from the Prep-HPLC separation was designated as Compound 130A (a mixture of two enantiomers, 2.2 mg, 0.003 mmol); The second eluent from the Prep-HPLC separation was designated as Compound 130B (1.5 mg, 0.0022 mmol, mixture of two enantiomers); Both are white solids.

Compound 130A: LC-MS: MS (ESI): 683 m/z [M+H]⁺, retention time: 1.82 minutes, purity: >99% (214 nm) (LC-MS Method 26). ¹H NMR (400 MHz, CD₃OD) δ 8.03-8.01 (m, 1H), 7.79 (s, 1H), 7.66-7.59 (m, 2H), 7.52-7.46 (m, 2H), 7.38 (d, J=11.2 Hz, 1H), 7.13 (t, J=7.5 Hz, 1H), 7.06 (d, J=7.6 Hz, 1H), 7.00 (s, 1H), 6.93-6.90 (m, 2H), 6.66-6.57 (m, 2H), 6.22 (d, J=14.8 Hz, 1H), 5.13 (dd, J=10.8, 4.4 Hz, 1H), 2.89-2.80 (m, 2H), 2.56-2.48 (m, 2H), 2.06-1.98 (m, 1H), 1.76-1.68 (m, 1H), 1.58-1.45 (m, 2H), 1.35-1.29 (m, 1H), 1.20 (s, 3H), 1.15 (s, 3H), 1.10-1.00 (m, 1H), 0.95-0.85 (m, 1H), 0.50-0.40 (m, 1H) ppm.

Compound 130B: LC-MS: MS (ESI): 683 m/z [M+H]⁺, retention time: 1.82 minutes, purity: >99% (214 nm) (LC-MS Method 26). ¹H NMR (400 MHz, CD₃OD) δ 8.07-7.99 (m, 1H), 7.85-7.75 (m, 1H), 7.70-7.60 (m, 2H), 7.52-7.44 (m, 2H), 7.30 (d, J=10.8 Hz, 1H), 7.22-7.18 (m, 1H), 7.15-7.08 (m, 2H), 7.02 (d, J=7.2 Hz, 1H), 6.99-6.85 (m, 1H), 6.72-6.68 (m, 1H), 6.55-6.45 (m, 1H), 6.30-6.20 (dm, 1H), 5.26-5.16 (m, 1H), 2.90-2.80 (m, 2H), 2.57-2.47 (m, 2H), 2.40-2.30 (m, 1H), 2.10-2.00 (m, 1H), 1.85-1.75 (m, 1H), 1.50-1.24 (m, 3H), 1.22 (s, 3H), 1.16 (s, 3H), 1.10-1.00 (m, 2H) ppm.

Example 131. Compound 131A. (2S)-2-methyl-3-[3-(9,9,24-trifluoro-3,6-dimethyl-11,26-dioxa-3,4,15,21,30,32,33-heptazahexacyclo[25.3.1.12,5.112,15.017,25.018,22]tritriaconta-1(31),2(33),4,12(32),13,17,19,22,24,27,29-undecaen-6-yl)phenyl]propanoic acid

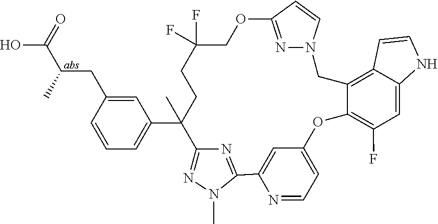 [SMILES: C[C@@H](Cc1cccc(C2(C)CCC(F)(F)COc3ccn(n3)Cc3c(c(F)cc4[nH]ccc34)Oc3ccnc(c3)-c3nc2nn3C)c1)C(=O)O]

and

Example 131B. (2S)-2-methyl-3-[3-(9,9,25-trifluoro-3,6-dimethyl-11,27-dioxa-3,4,15,16,22,31,33-heptazahexacyclo[26.3.1.12,5.012,16.018,26.019,23]tritriaconta-1(32),2(33),4,12,14,18,20,23,25,28,30-undecaen-6-yl)phenyl]propanoic acid

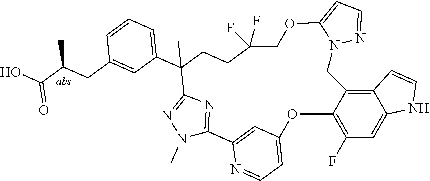 [SMILES: C[C@@H](Cc1cccc(C2(C)CCC(F)(F)COc3ccnn3Cc3c(c(F)cc4[nH]ccc34)Oc3ccnc(c3)-c3nc2nn3C)c1)C(=O)O]

Exchanging ethyl 3-(3-(6-((1H-pyrazol-3-yl)oxy)-2,5,5-trimethyl-1-(2-methylhydrazineyl)-1-oxohexan-2-yl)phenyl)-2-methylpropanoate (Intermediate 83-5) with methyl (2S)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (Intermediate 101, 200 mg, 0.442 mmol); and (6-fluoro-5-(4-fluoro-3-(imino(methylthio)methyl)-phenoxy)-1-(phenylsulfonyl)-1H-indol-4-yl)methyl acetate hydroiodide (Intermediate 82-3) with methyl 4-((6-fluoro-4-(hydroxymethyl)-1-tosyl-1H-indol-5-yl)oxy)pyridine-2-carbimidothioate (Intermediate 113, 271 mg, 0.442 mmol), the reaction procedure sequence (Steps A, C and D) described for Example 99A & 99B, 99A & 99B was used to prepare the title compounds (190 mg). The two regio-isomers were separated by silica gel column (eluting with 0-50% ethyl acetate in petroleum ether) after corresponding Step C of Example 99A & 99B, 99A & 99B. The first eluent, after hydrolysis and deprotection (corresponding Step D), was designated as Compound 131A (14 mg, 0.016 mmol, 10%). The second eluent, after hydrolysis and deprotection, was designated as Compound 131B (5.4 mg, 0.0075 mmol). Both products are pale yellow oil.

Compound 131A: LC-MS: MS (ESI): 686 m/z [M+H]⁺, retention time: 1.93 minutes, purity: 99% (214 nm) (LC-MS Method 26). ¹H NMR (500 MHz, CD₃OD) δ 8.50 (d, J=5.5 Hz, 1H), 7.36 (d, J=3.2 Hz, 1H), 7.27-7.24 (m, 1H), 7.19-7.17 (m, 1H), 7.14-7.07 (m, 2H), 7.02-6.92 (m, 3H), 6.80 (s, 1H), 6.74 (d, J=3.1 Hz, 1H), 5.44-5.36 (m, 2H), 5.04-5.00 (m, 1H), 4.28-4.18 (m, 4H), 3.80-3.72 (m, 1H), 2.92-2.84 (m, 1H), 2.62-2.50 (m, 2H), 2.30-2.20 (m, 1H), 2.18-2.05 (m, 1H), 2.05-1.90 (m, 1H), 1.61 (s, 3H), 1.35-1.25 (m, 1H), 1.03-1.01 (m, 3H) ppm.

Compound 131B: LC-MS: MS (ESI): 686 m/z [M+H]⁺, retention time: 1.96 minutes, purity: 92% (214 nm) (LC-MS Method 26). ¹H NMR (400 MHz, CD₃OD) δ 8.62 (d, J=5.4 Hz, 1H), 7.37-7.29 (m, 2H), 7.26-7.22 (m, 2H), 7.17-7.10 (m, 2H), 7.00-6.85 (m, 3H), 5.75 (s, 1H), 5.56-5.44 (m, 2H), 5.28 (s, 1H), 4.29 (s, 3H), 4.10-4.00 (m, 2H), 2.93-2.82 (m, 1H), 2.57-2.50 (m, 2H), 2.25-2.20 (m, 1H), 2.08-1.95 (m, 1H), 1.69 (s, 3H), 1.34-1.21 (m, 2H), 1.06-0.95 (m, 3H) ppm.

Example 132. Compound 132A. (2R)-2-methyl-3-[3-(9,9,24-trifluoro-3,6-dimethyl-11,26-dioxa-3,4,15,21,30,32,33-heptazahexacyclo[25.3.1.12,5.112,15.017,25.018,22]tritriaconta-1(31),2(33),4,12(32),13,17,19,22,24,27,29-undecaen-6-yl)phenyl]propanoic acid

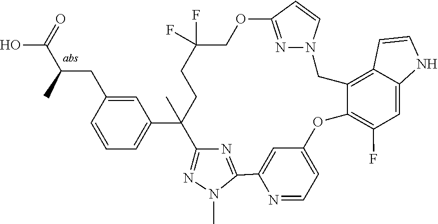 [SMILES: C[C@H](Cc1cccc(C2(C)CCC(F)(F)COc3ccn(n3)Cc3c(c(F)cc4[nH]ccc34)Oc3ccnc(c3)-c3nc2nn3C)c1)C(=O)O]

and

Example 132B. (2R)-2-methyl-3-[3-(9,9,25-trifluoro-3,6-dimethyl-11,27-dioxa-3,4,15,16,22,31,33-heptazahexacyclo[26.3.1.12,5.012,16.018,26.019,23]tritriaconta-1(32),2(33),4,12,14,18,20,23,25,28,30-undecaen-6-yl)phenyl]propanoic acid

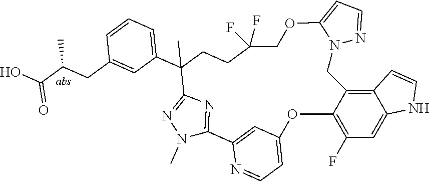 [SMILES: C[C@H](Cc1cccc(C2(C)CCC(F)(F)COc3ccnn3Cc3c(c(F)cc4[nH]ccc34)Oc3ccnc(c3)-c3nc2nn3C)c1)C(=O)O]

Exchanging ethyl 3-(3-(6-((1H-pyrazol-3-yl)oxy)-2,5,5-trimethyl-1-(2-methylhydrazineyl)-1-oxohexan-2-yl)phenyl)-2-methylpropanoate (Intermediate 83-5) with methyl (2R)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (Intermediate 101-1, 110 mg, 0.24 mmol); and (6-fluoro-5-(4-fluoro-3-(imino(methylthio)methyl)-phenoxy)-1-(phenylsulfonyl)-1H-indol-4-yl)methyl acetate hydroiodide (Intermediate 82-3) with methyl 4-((6-fluoro-4-(hydroxymethyl)-1-tosyl-1H-indol-5-yl)oxy)pyridine-2-carbimidothioate (Intermediate 113, 118 mg, 0.24 mmol), the reaction procedure sequence (Steps A, C and D) described for Example 99A & 99B, 99A & 99B was used to prepare the title compounds. The two regio-isomers were separated by silica gel column (eluting with 0-50% ethyl acetate in petroleum ether) after corresponding Step C of Example 99A & 99B, 99A & 99B. The first eluent, after hydrolysis and deprotection (corresponding Step D), was designated as Compound 132A (2.8 mg, 0.00389 mmol). The second eluent, after hydrolysis and deprotection (corresponding Step D), was designated as Compound 132B (5.4 mg, 0.0075 mmol). Both products are pale yellow oil.

Compound 132A: LC-MS: MS (ESI): 686 m/z [M+H]⁺, retention time: 1.92 minutes, purity: >99% (214 nm) (LC-MS Method 26). ¹H NMR (500 MHz, CD₃OD) δ 8.50 (d, J=5.5 Hz, 1H), 7.36 (d, J=3.0 Hz, 1H), 7.27-7.24 (m, 1H), 7.19-7.18 (m, 1H), 7.14-7.07 (m, 2H), 7.02-6.95 (m, 3H), 6.80 (s, 1H), 6.74 (d, J=3.0 Hz, 1H), 5.44-5.36 (m, 2H), 5.04-5.00 (m, 1H), 4.28 (d, J=4.0 Hz, 3H), 4.27-4.37 (m, 1H), 3.81-3.75 (m, 1H), 2.92-2.84 (m, 1H), 2.62-2.50 (m, 2H), 2.30-2.22 (m, 1H), 2.18-2.08 (m, 1H), 2.02-1.96 (m, 1H), 1.61 (s, 3H), 1.35-1.27 (m, 1H), 1.03-1.01 (m, 3H) ppm.

Compound 132B: LC-MS: MS (ESI): 686 m/z [M+H]⁺, retention time: 1.97 minutes, purity: 99% (214 nm) (LC-MS Method 26). ¹H NMR (500 MHz, CD₃OD) δ 8.62 (d, J=5.5 Hz, 1H), 7.36-7.32 (m, 2H), 7.25-7.22 (m, 2H), 7.18-7.09 (m, 2H), 6.98-6.86 (m, 3H), 5.76-5.74 (m, 1H), 5.54-5.45 (m, 2H), 5.04-5.00 (m, 1H), 4.28 (s, 3H), 4.12-3.98 (m, 2H), 2.92-2.84 (m, 1H), 2.62-2.50 (m, 2H), 2.26-2.17 (m, 1H), 2.03-1.90 (m, 1H), 1.80-1.70 (m, 1H), 1.69 (s, 3H), 1.37-1.28 (m, 1H), 1.02-0.98 (m, 3H) ppm.

Example 133. Compound 133. (2S)-2-methyl-3-[3-(9,9,24-trifluoro-3,6-dimethyl-26-oxo-11-oxa-26λ4-thia-3,4,15,21,30,32,33-heptazahexacyclo[25.3.1.12,5.112,15.017,25.018,22]tritriaconta-1(31),2(33),4,12(32),13,17,19,22,24,27,29-undecaen-6-yl)phenyl]propanoic acid

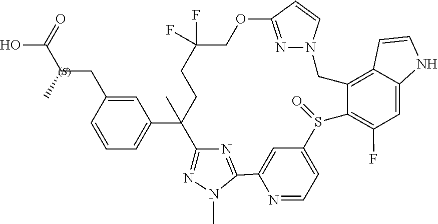

Exchanging ethyl 3-(3-(6-((1H-pyrazol-3-yl)oxy)-2,5,5-trimethyl-1-(2-methylhydrazineyl)-1-oxohexan-2-yl)phenyl)-2-methylpropanoate (Intermediate 83-5) with methyl (2S)-3-(3-(6-((1H-pyrazol-3-yl)oxy)-5,5-difluoro-2-methyl-1-(2-methylhydrazineyl)-1-oxohexan-2-yl)phenyl)-2-methylpropanoate (Intermediate 101, 400 mg, 0.884 mmol); and (6-fluoro-5-(4-fluoro-3-(imino(methylthio)methyl)-phenoxy)-1-(phenylsulfonyl)-1H-indol-4-yl)methyl acetate hydroiodide (Intermediate 82-3) with methyl 4-((6-fluoro-4-(hydroxymethyl)-1-tosyl-1H-indol-5-yl)thio)pyridine-2-carbimidothioate (Intermediate 113-1, 557 mg, 0.884 mmol), the reaction procedure sequence (Steps A, C) described for Example 99A & 99B, 99A & 99B, followed by Step B of Example 124A and 124B, then deprotected and hydrolyzed, as described in Step C of Example 99A & 99B, 99A & 99B was used to prepare the title compound 133 (0.40 mg, 0.0006 mmol). The other regio-isomer was lost after silica gel column chromatography.

Compound 133: LC-MS: MS (ESI): 718 m/z [M+H]⁺, retention time: 1.77 minutes, purity: 97% (214 nm) (LC-MS Method 26).

Example 134. Compound 134A and Compound 134B. Diastereomer 1 and 2 of (2S)-3-[3-(24-Fluoro-11,11-dimethyl-26-oxo-26λ4-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl)phenyl]-2-methyl-propanoic acid

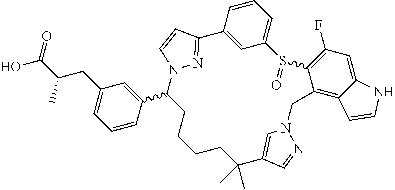 [SMILES: C[C@@H](Cc1cccc(C2CCCCC(C)(C)c3cnn(c3)Cc3c(c(F)cc4[nH]ccc34)S(=O)c3cccc(c3)-c3ccn2n3)c1)C(=O)O]

6-(3-Bromophenyl)-24-fluoro-11,11-dimethyl-21-(p-tolylsulfonyl)-26-thia-5,13,14,15,21,33-hexazahexacyclo[25.3.1.12,5.112,15.017,25.018,22]tritriaconta-1(31),2(33),3,12(32),13,17,19,22,24,27,29-undecaene

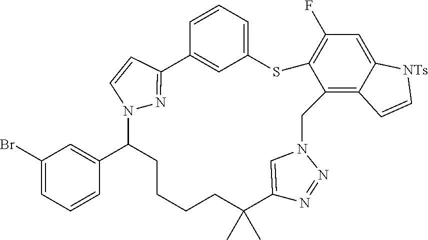 [SMILES: Cc1ccc(S(=O)(=O)n2ccc3c4c(c(F)cc32)Sc2cccc(c2)-c2ccn(n2)C(c2cccc(Br)c2)CCCCC(C)(C)c2cn(nn2)C4)cc1]

Step A: Exchanging (6-fluoro-5-((4-fluoro-3-(1H-pyrazol-3-yl)phenyl)thio)-1-tosyl-1H-indol-4-yl)methanol (Intermediate 55-5) with (5-((3-(1H-pyrazol-3-yl)phenyl)thio)-6-fluoro-1-tosyl-1H-indol-4-yl)methanol (Intermediate 116, 1300 mg, 1.16 mmol) and 1-bromo-3-(1-bromo-3-((2,2-dimethylbut-3-yn-1-yl)oxy)propyl)benzene (Intermediate 98) with 1-bromo-3-(1-bromo-6,6-dimethyloct-7-yn-1-yl)benzene (Intermediate 112, 561 mg, 1.51 mmol), the reaction procedure sequence (Steps A to C) described for Example 129A and 129B was used to prepare the title compound (230 mg, 0.261 mmol) as a yellow solid. LC-MS: MS (ESI): 809, 811 m/z [M+H]⁺, retention time: 2.58 minutes, purity: 92% (214 nm) (LC-MS Method 2). 24-Fluoro-6-(3-iodophenyl)-11,11-dimethyl-21-(p-tolylsulfonyl)-26-thia-5,13,14,15,21,33-hexazahexacyclo[25.3.1.12,5.112,15.017,25.018,22]tritriaconta-1(31),2(33),3,12(32),13,17,19,22,24,27,29-undecaene

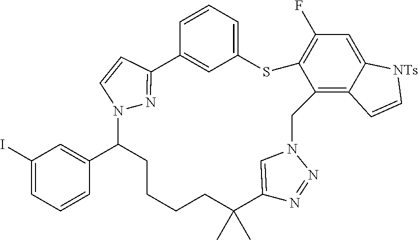 [SMILES: Cc1ccc(S(=O)(=O)n2ccc3c4c(c(F)cc32)Sc2cccc(c2)-c2ccn(n2)C(c2cccc(I)c2)CCCCC(C)(C)c2cn(nn2)C4)cc1]

Step B: To a stirred solution of Step A product (200 mg, 0.247 mmol) in 1,4-dioxane (15 mL) were added N,N′-dimethyl-cyclohexane-1,2-diamine (3.5 mg, 0.025 mmol), copper(I) iodide (2.4 mg, 0.012 mmol), and sodium iodide (74 mg, 0.49 mmol). The mixture was stirred at 110° C. for 16 hours, cooled to room temperature, and filtered. The filter cake was washed with 1, 4-dioxane (10 mL). The filtrate was concentrated. The residue was purified by automated flash chromatography (20 g silica gel column, eluting with 15-40% ethyl acetate in petroleum ether) to afford the title compound (210 mg, 0.23 mmol, 91%) as a yellow solid. LC-MS: MS (ESI): 857 m/z [M+H]⁺, retention time: 2.58 minutes, purity: 93% (214 nm) (LC-MS Method 28).

Methyl (2S)-3-[3-[124-fluoro-11,11-dimethyl-21-(p-tolylsulfonyl)-26-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl]phenyl]-2-methyl-propanoate

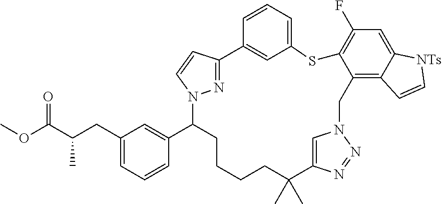 [SMILES: COC(=O)[C@@H](C)Cc1cccc(C2CCCCC(C)(C)c3cn(nn3)Cc3c(c(F)cc4c3ccn4S(=O)(=O)c3ccc(C)cc3)Sc3cccc(c3)-c3ccn2n3)c1]

Step C: To a stirred suspension of Zinc (78 mg, 1.19 mmol) in tetrahydrofuran (10 mL) was added iodine (5.0 mg, 0.0198 mmol). The mixture was stirred at room temperature for 40 minutes, then treated with methyl (2R)-3-iodo-2-methyl-propanoate (68 mg, 0.298 mmol). The reaction mixture was stirred at room temperature for 40 minutes. To this solution was added bis(tri-tert-butylphosphine)palladium(0) (10 mg, 0.0198 mmol) and Step B product (170 mg, 0.198 mmol). The solution was stirred at room temperature for 16 hours, diluted with ethyl acetate (30 mL), washed with brine, dried over sodium sulfate, filtered, and concentrated. The residue was purified by prep-TLC (silica gel, developed by petroleum ether/ethyl acetate=2/1, Rf=0.4) to give the title compound (100 mg, 0.112 mmol, 56%) as a colorless oil. LC-MS: MS (ESI): 831 m/z [M+H]⁺, retention time: 2.46 minutes, purity: 93% (214 nm) (LC-MS Method 2).

Compound 134. (2S)-3-[3-(24-Fluoro-11,11-dimethyl-26-oxo-26A4-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl)phenyl]-2-methyl-propanoic acid

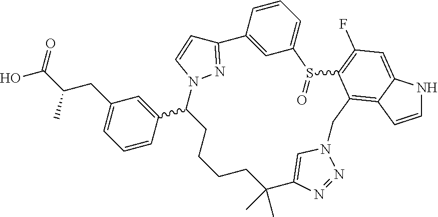 [SMILES: C[C@@H](Cc1cccc(C2CCCCC(C)(C)c3cn(nn3)Cc3c(c(F)cc4[nH]ccc34)S(=O)c3cccc(c3)-c3ccn2n3)c1)C(=O)O]

Step D: Exchanging Ethyl 3-[3-[24,30-difluoro-11,11-dimethyl-21-(p-tolylsulfonyl)-9-oxa-26-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl]phenyl]propanoate (Step C product of Example 129A and 129B with Methyl (2S)-3-[3-[24-fluoro-11,11-dimethyl-21-(p-tolylsulfonyl)-26-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl]phenyl]-2-methyl-propanoate (Step C product of this Example, 90 mg, 0.101 mmol), the reaction procedure sequence (Steps F and G) described for Example 129A and 129B was used to prepare the title compounds. The two diastereomers were separated by pre-HPLC at the corresponding Step F of Examples 129A and 129B. The First eluent, after hydrolysis and deprotection, as described in corresponding Step G of Example 129A and 129B, was designated as Compound 134A (8.7 mg, 0.0128 mmol); The second eluent, after hydrolysis and deprotection, was designated as Compound 134B (4.6 mg, 0.0068 mmol). Both were obtained as white solid.

Compound 134A: LC-MS: MS (ESI): 679 m/z [M+H]⁺, retention time: 1.90 minutes, purity: >99% (214 nm) (LC-MS Method 26). ¹H NMR (500 MHz, CDCl₃) δ 9.83 (bs, 1H), 8.02-7.93 (m, 1H), 7.70-7.60 (m, 3H), 7.05-7.00 (m, 2H), 6.96-6.94 (m, 2H), 6.90-6.83 (m, 3H), 6.71-6.55 (m, 1H), 6.50-6.42 (m, 2H), 6.36-6.20 (m, 1H), 4.98 (d, J=9.0 Hz, 1H), 2.86-2.71 (m, 2H), 2.59-2.57 (m, 2H), 2.47-2.31 (m, 2H), 2.24-2.12 (m, 1H), 1.92-1.80 (m, 1H), 1.60-1.51 (m, 1H), 1.48-1.36 (m, 1H), 1.20 (s, 3H), 1.05-0.97 (m, 7H), 0.68-0.64 (m, 1H) ppm.

Compound 134B: LC-MS: MS (ESI): 679 m/z [M+H]⁺, retention time: 1.90 minutes, purity: >99% (214 nm) (LC-MS Method 26). ¹H NMR (500 MHz, CDCl₃) δ 9.82-9.70 (m, 1H), 8.03 (bs, 1H), 7.69-7.60 (m, 2H), 7.60-7.40 (m, 1H), 7.22 (d, J=10.5 Hz, 1H), 7.10-7.04 (m, 1H), 7.01-6.93 (m, 1H), 6.91-6.85 (m, 1H), 6.64-6.53 (m, 3H), 6.48-6.28 (m, 3H), 5.75 (bs, 1H), 5.33-5.26 (m, 1H), 2.55-2.51 (m, 2H), 2.28-2.23 (m, 2H), 2.17-2.12 (m, 2H), 2.08-2.00 (m, 2H), 1.67-1.62 (m, 2H), 1.44-1.40 (m, 1H), 1.40-1.20 (m, 7H), 0.88 (s, 3H).

Example 135. Compound 135A and Compound 135B. Diastereomers 1 and 2 of (2R)-3-[3-(24,30-Difluoro-11,11-dimethyl-26-oxo-9-oxa-26λ4-thia-5,13,14,15,21,33-hexazahexacyclo[25.3.1.12,5.112,15.017,25.018,22]tritriaconta-1(31),2(33),3,12(32),13,17,19,22,24,27,29-undecaen-6-yl)phenyl]-2-methyl-propanoic acid

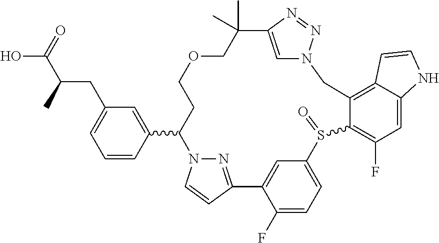 [SMILES: C[C@H](Cc1cccc(C2CCOCC(C)(C)c3cn(nn3)Cc3c(c(F)cc4[nH]ccc34)S(=O)c3ccc(F)c(c3)-c3ccn2n3)c1)C(=O)O]

Exchanging (5-((3-(1H-pyrazol-3-yl)phenyl)thio)-6-fluoro-1-tosyl-1H-indol-4-yl)methanol (Intermediate 116) with (6-fluoro-5-((4-fluoro-3-(1H-pyrazol-3-yl)phenyl)thio)-1-tosyl-1H-indol-4-yl)methanol (Intermediate 55-5, 650 mg, 1.27 mmol), and 1-bromo-3-(1-bromo-6,6-dimethyloct-7-yn-1-yl)benzene (Intermediate 112) with 1-bromo-3-(1-bromo-3-((2,2-dimethylbut-3-yn-1-yl)oxy)propyl)benzene (Intermediate 98, 570 mg, 1.52 mmol), the reaction procedure sequence described for Example 134A and 134B (Steps A to D) was used to prepare the title compounds. The first eluent from prep_HPLC separation of sulfoxide formation step, after deprotection and hydrolysis, was designated as Compound 135A (11 mg, white solid, mixture of two diastereomers); The second eluent was designated as Compound 135B (18 mg, white solid, mixture of two diastereomers).

Compound 135A: LC-MS: MS (ESI): 699 m/z [M+H]⁺, retention time: 1.76 minutes, purity: >99% (214 nm) (LC-MS Method 26). ¹H NMR (500 MHz, CD₃OD) δ 7.90 (s, 1H), 7.70 (s, 1H), 7.65-7.55 (m, 1H), 7.50-7.35 (m, 3H), 7.20-7.15 (m, 1H), 7.15-7.05 (m, 1H), 7.02 (s, 1H), 6.95-6.90 (d, J=7.4 Hz, 1H), 6.75 (s, 1H), 6.60-6.50 (m, 2H), 6.30-6.25 (m, 1H), 5.35-5.28 (m 1H), 3.40-3.3 (m, 1H), 3.15-3.12 (m, 1H), 2.95-2.90 (m, 1H), 2.68-2.53 (m, 2H), 2.34 (s, 1H), 1.92 (bs, 1H), 1.40-1.30 (m, 1H), 1.20 (s, 3H), 1.15 (d, J=4.4 Hz, 1H), 1.10-0.90 (m, 7H) ppm.

Compound 135B: LC-MS: MS (ESI): 699 m/z [M+H]⁺, retention time: 1.78 minutes, purity: >99% (214 nm) (LC-MS Method 26). ¹H NMR (500 MHz, CD₃OD) δ 7.97 (s, 1H), 7.8-7.70 (m, 1H), 7.63 (s, 2H), 7.47-7.30 (m, 4H), 7.21 (t, J=7.6 Hz, 1H), 7.10 (d, J=7.7 Hz, 1H), 7.05 (s, 1H), 7.02 (d, J=6.9 Hz, 1H), 6.64 (s, 1H), 6.39-6.28 (m, 2H), 5.42 (d, J=10 Hz, 1H), 3.50-3.42 (m, 1H), 3.30-3.20 (m, 1H), 3.16-3.10 (m, 1H), 3.00-2.92 (m, 1H), 2.68-2.56 (m, 2H), 2.45-2.35 (m 1H), 2.20-2.10 (m, 1H), 1.35-1.20 (m, 1H), 1.20-0.92 (m, 9H) ppm.

Example 136. Compound 136. (2S)-3-[3-(24-Fluoro-11,11-dimethyl-26-oxa-5,13,14,15,21,30,33-heptazahexacyclo[25.3.1.12,5.112,15.017,25.018,22]tritriaconta-1(31),2(33),3,12(32),13,17,19,22,24,27,29-undecaen-6-yl)phenyl]-2-methyl-propanoic acid

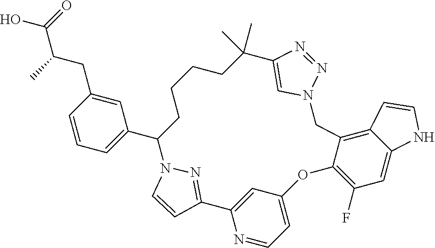 [SMILES: C[C@@H](Cc1cccc(C2CCCCC(C)(C)c3cn(nn3)Cc3c(c(F)cc4[nH]ccc34)Oc3ccnc(c3)-c3ccn2n3)c1)C(=O)O]

Step A: Exchanging (6-fluoro-5-((4-fluoro-3-(1H-pyrazol-3-yl)phenyl)thio)-1-tosyl-1H-indol-4-yl)methanol (Intermediate 55-5) with ((5-((2-(1H-pyrazol-3-yl)pyridin-4-yl)oxy)-6-fluoro-1-tosyl-1H-indol-4-yl)methanol (Intermediate 116-1, 1.70 g, 3.55 mmol)) the reaction procedure sequence (Steps A to C) described for Example 129A and 129B, followed by Step C of Example 134A and 134B, then Step G of Example 129A and 129B, was used to prepare the title compound (6.9 mg, 0.0107 mmol, mixture of two diastereomers) as a yellow solid. LC-MS: MS (ESI): 648 m/z [M+H]⁺, retention time: 1.61 minutes, purity: >99% (214 nm) (LC-MS Method 8). ¹H NMR (400 MHz, CD₃OD) δ 8.43 (d, J=6.0 Hz, 1H), 7.63 (s, 1H), 7.45-7.40 (m, 2H), 7.31 (s, 1H), 7.26-7.23 (m, 1H), 7.16 (t, J=7.2 Hz, 1H), 7.10 (s, 1H), 7.07-7.03 (m, 2H), 6.90-6.83 (m, 2H), 5.95-5.85 (m, 2H), 5.21 (d, J=11.6 Hz, 1H), 3.23-2.19 (m, 1H), 2.96-2.91 (m, 1H), 2.65-2.54 (m, 2H), 2.27-2.18 (m, 1H), 1.95-1.85 (m, 1H), 1.49-1.42 (m, 1H), 1.37-1.28 (m, 3H), 1.09 (s, 3H), 1.05 (d, J=6.0 Hz, 3H), 1.00 (s, 3H), 0.89-0.85 (m, 1H), 0.67-0.60 (m, 1H) ppm.

Example 137. Compound 137. (2R)-3-[3-(24-fluoro-11,11-dimethyl-26-oxa-5,13,14,15,21,30,33-heptazahexacyclo[25.3.1.12,5.112,15.017,25.018,22]tritriaconta-1(31),2(33),3,12(32),13,17,19,22,24,27,29-undecaen-6-yl)phenyl]-2-methyl-propanoic acid

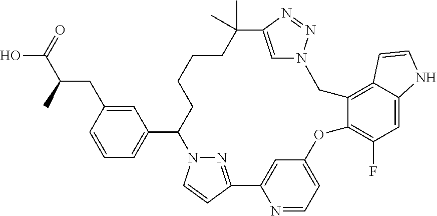 [SMILES: C[C@H](Cc1cccc(C2CCCCC(C)(C)c3cn(nn3)Cc3c(c(F)cc4[nH]ccc34)Oc3ccnc(c3)-c3ccn2n3)c1)C(=O)O]

Step A: Exchanging (6-fluoro-5-((4-fluoro-3-(1H-pyrazol-3-yl)phenyl)thio)-1-tosyl-1H-indol-4-yl)methanol (Intermediate 55-5) with ((5-((2-(1H-pyrazol-3-yl)pyridin-4-yl)oxy)-6-fluoro-1-tosyl-1H-indol-4-yl)methanol (Intermediate 116-1, 1.70 g, 3.55 mmol)) the reaction procedure sequence (Steps A to C) described for Example 129A and 129B, followed by Step C of Example 134A and 134B (Exchanging methyl (2R)-3-iodo-2-methyl-propanoate to methyl (2S)-3-iodo-2-methyl-propanoate (39 mg, 0.17 mmol) in this step), then Step G of Example 129A and 129B, was used to prepare the title compound (18 mg, 0.0273 mmol, mixture of two diastereomers) as a white solid. LC-MS: MS (ESI): 648 m/z [M+H]⁺, retention time: 1.61 minutes, purity: >99% (214 nm) (LC-MS Method 8). ¹H NMR (400 MHz, CD₃OD) δ 8.44 (d, J=6.0 Hz, 1H), 7.62 (s, 1H), 7.45-7.41 (m, 2H), 7.31 (s, 1H), 7.25-7.24 (m, 1H), 7.16 (t, J=7.6 Hz, 1H), 7.10 (s, 1H), 7.06-7.03 (m, 3H), 6.88-6.86 (m, 2H), 5.95-5.85 (m, 2H), 5.22 (d, J=10.8 Hz, 1H), 2.96-2.90 (m, 1H), 2.64-2.56 (m, 2H), 2.24-2.22 (m, 1H), 1.94-1.86 (m, 1H), 1.50-1.42 (m, 1H), 1.40-1.35 (m, 2H), 1.20-1.00 (m, 10H), 0.97-0.86 (m, 1H), 0.64-0.61 (m, 1H).

Example 138. Compound 138. (2S)-3-[3-(24-fluoro-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl)phenyl]-2-methyl-propanoic acid

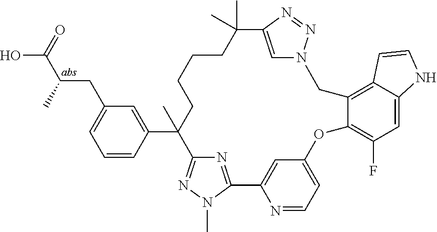 [SMILES: C[C@@H](Cc1cccc(C2(C)CCCCC(C)(C)c3cn(nn3)Cc3c(c(F)cc4[nH]ccc34)Oc3ccnc(c3)-c3nc2nn3C)c1)C(=O)O]

(6R)-24-fluoro-6-(3-iodophenyl)-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaene

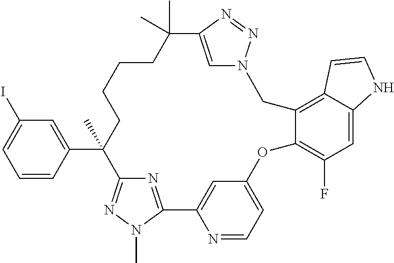 [SMILES: Cn1nc2nc1-c1cc(ccn1)Oc1c(F)cc3[nH]ccc3c1Cn1cc(nn1)C(C)(C)CCCC[C@]2(C)c1cccc(I)c1]

Step A: Exchanging (6-fluoro-5-(4-fluoro-3-(imino(methylthio)methyl)phenoxy)-1-(phenylsulfonyl)-1H-indol-4-yl)methyl acetate hydroiodide (Intermediate 82-3) with methyl 4-((6-fluoro-4-(hydroxymethyl)-1H-indol-5-yl)oxy)pyridine-2-carbimidothioate (Intermediate 117, 8.5 g, 25.8 mmol), and 2-(3-bromophenyl)-N′,2-dimethyl-5-((2-methylbut-3-yn-2-yl)oxy)pentanehydrazide (Intermediate 83-4) with (R)-2-(3-iodophenyl)-N′,2,7,7-tetramethylnon-8-ynehydrazide (Intermediate 83-8, 11.0 g, 25.8 mmol), the reaction procedure sequence (Steps A, B, and C described for Example 97A and 97B, followed by Step B described for Example 704) was followed to prepare the title compound (4.1 g, 5.7 mmol). LC-MS: MS (ESI): 717 m/z [M+H]⁺.

Methyl (2S)-3-[3-[(6R)-24-fluoro-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]-2-methyl-propanoate

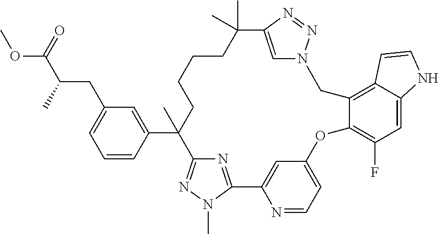 [SMILES: COC(=O)[C@@H](C)Cc1cccc(C2(C)CCCCC(C)(C)c3cn(nn3)Cc3c(c(F)cc4[nH]ccc34)Oc3ccnc(c3)-c3nc2nn3C)c1]

Step B: To a stirred suspension of zinc (928 mg, 14.3 mmol) in N,N-dimethylformamide (20 mL) was added iodine (61 mg, 0.24 mmol). The mixture was stirred at room temperature for 40 min, then treated with methyl (2R)-3-iodo-2-methyl-propanoate (1.35 g, 5.95 mmol). The mixture was stirred at room temperature for 40 minutes. To this mixture was added 2-dicyclohexylphosphino-2′,6′-dimethoxybiphenyl (98 mg, 0.24 mmol), tris(dibenzylideneacetone)dipalladium (108 mg, 0.12 mmol), and Step A product (1.7 g, 2.38 mmol). The reaction mixture was stirred at room temperature overnight, diluted with water (30 mL) and ethyl acetate (30 mL). The solid was removed by filtration. The filtrate extracted with ethyl acetate (3×20 mL). The combined organic layers were washed with brine (20 mL), dried over sodium sulfate, filtered, and concentrated. The residue was purified by flash chromatography (40 g silica gel column, eluting with 0-30% ethyl acetate in petroleum ether) to give the title compound (1.2 g, 1.7 mmol, 71%) as light-yellow oil. LC-MS: MS (ESI): 691 m/z [M+H]⁺, retention time: 2.19 minutes, purity: 94% (214 nm) (LC-MS Method 26).

Compound 138: (2S)-3-[3-[(6R)-24-fluoro-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]-2-methyl-propanoic acid

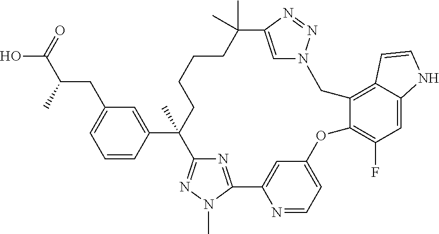 [SMILES: C[C@@H](Cc1cccc([C@@]2(C)CCCCC(C)(C)c3cn(nn3)Cc3c(c(F)cc4[nH]ccc34)Oc3ccnc(c3)-c3nc2nn3C)c1)C(=O)O]

Step C: To a stirred suspension of Step B product (1.2 g, 1.7 mmol) in tetrahydrofuran (9 mL), methanol (3 mL) and water (3 mL) was added lithium hydroxide (163 mg, 6.8 mmol). The reaction mixture was stirred at room temperature overnight, diluted with water (20 mL) and acidified with 1 N hydrochloric acid. The solution was extracted with ethyl acetate (3×20 mL). The combined organic layers were washed with brine, dried over sodium sulfate, filtered, and concentrated. The residue was purified by reverse phase Prep-HPLC to give the title compound (614 mg, 0.90 mmol, 53%) as a white solid. LC-MS: MS (ESI): 677 m/z [M+H]⁺, retention time: 1.96 minutes, purity: >99% (214 nm) (LC-MS Method 26). ¹H NMR (400 MHz, CD₃OD) δ 8.63 (d, J=5.6 Hz 1H), 7.45 (s, 1H), 7.41 (d, J=3.2 Hz, 1H), 7.37-7.29 (m, 2H), 7.14 (d, J=2.4 Hz, 1H), 7.09 (t, J=7.6 Hz, 1H), 6.99 (s, 1H), 6.97-6.91 (m, 2H), 6.76 (d, J=2.4 Hz, 1H), 5.99 (d, J=14.6 Hz, 1H), 5.76 (d, J=14.2 Hz, 1H), 4.35 (s, 3H), 2.94-2.86 (m, 1H), 2.62-2.49 (m, 2H), 2.02-1.98 (m, 2H), 1.55 (s, 3H), 1.53-1.36 (m, 2H), 1.26-1.15 (m, 1H), 1.12 (s, 3H), 1.10-0.99 (m, 7H), 0.63-0.57 (m, 2H) ppm.

Example 139. Compound 139. (2R)-3-[3-[(6R)-24-fluoro-3,6,11,11-tetramethyl-26-oxo-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoic acid

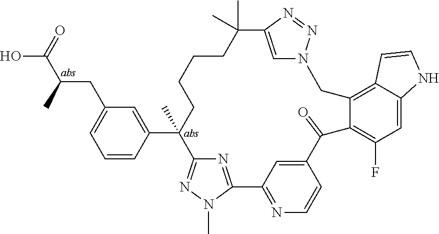 [SMILES: C[C@H](Cc1cccc([C@@]2(C)CCCCC(C)(C)c3cn(nn3)Cc3c(c(F)cc4[nH]ccc34)C(=O)c3ccnc(c3)-c3nc2nn3C)c1)C(=O)O]

Methyl (2R)-3-[3-[(6R)-24-fluoro-3,6,11,11-tetramethyl-21-(p-tolylsulfonyl)-26-tetrahydropyran-2-yloxy-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoate

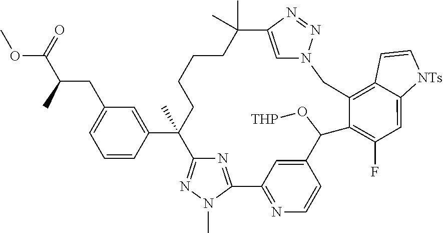

Step A: Exchanging (6-fluoro-5-(4-fluoro-3-(imino(methylthio)methyl)phenoxy)-1-(phenylsulfonyl)-1H-indol-4-yl)methyl acetate hydroiodide (Intermediate 82-3) with methyl 4-((6-fluoro-4-(hydroxymethyl)-1-tosyl-1H-indol-5-yl)((tetrahydro-2H-pyran-2-yl)oxy)methyl)pyridine-2-carbimidothioate (Intermediate 117-1, 0.19 g, 0.326 mmol), and 2-(3-bromophenyl)-N′,2-dimethyl-5-((2-methylbut-3-yn-2-yl)oxy)pentanehydrazide (Intermediate 83-4) with (R)-2-(3-iodophenyl)-N′,2,7,7-tetramethylnon-8-ynehydrazide (Intermediate 83-8. 11.0 g, 25.8 mmol), the reaction procedure sequence (Steps A, C described for Example 97A and 97B, followed by Step B of Example 704, in this order) was used to prepare the title compound as a mixture of 4 diastereomers (79 mg). LC-MS: MS (ESI): 965 m/z [M+Na]⁺, retention time: 2.64+2.69+2.73+2.82 minutes, purity: 30+22+30+18% (214 nm) (LC-MS Method 8).

Methyl (2R)-3-[3-[(6R)-24-fluoro-3,6,11,11-tetramethyl-26-tetrahydropyran-2-yloxy-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoate

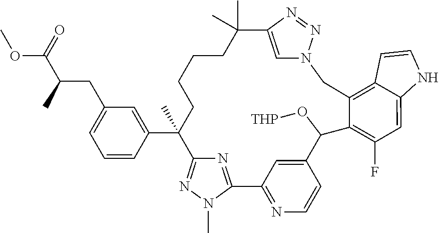

Step B: To a stirred solution of Step A product (42 mg, 0.044 mmol) in tetrahydrofuran (5 mL) was added tetra-n-butylammonium fluoride (1M in tetrahydrofuran) (0.089 mL, 0.089 mmol). The reaction mixture was stirred at room temperature for 1 hour and diluted with ethyl acetate (20 mL). The solution was washed with water, dried over sodium sulfate, filtered, and concentrated to give the title compound (35 mg, 99%) as an inseparable diastereomer mixture. LC-MS: MS (ESI): 789 m/z [M+H]⁺, retention time: 2.28 minutes, purity: 71% (214 nm) (LC-MS Method 8).

Methyl (2R)-3-[3-[(6R)-24-fluoro-26-hydroxy-3,6,11,11-tetramethyl-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoate

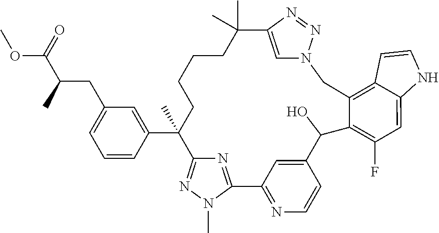 [SMILES: COC(=O)[C@H](C)Cc1cccc([C@@]2(C)CCCCC(C)(C)c3cn(nn3)Cc3c(c(F)cc4[nH]ccc34)C(O)c3ccnc(c3)-c3nc2nn3C)c1]

Step C: To a stirred solution of Step B product (35 mg, 0.044 mmol) in methanol (5 mL) was added pyridinium p-toluenesulfonate (45 mg, 0.18 mmol). The reaction mixture was stirred at 60° C. for 2 hours, cooled to room temperature and diluted with ethyl acetate (20 mL). The solution was washed with water, dried over sodium sulfate, filtered, and concentrated. The residue was purified by automated flash chromatography (4 g silica gel column, eluting with 0-100% ethyl acetate in petroleum ether) to give the title compound (26 mg, 83%) as a solid. LC-MS: MS (ESI): 705 m/z [M+H]⁺, retention time: 2.06 minutes, purity: 57% (214 nm) (LC-MS Method 8).

Methyl (2R)-3-[3-[(6R)-24-fluoro-3,6,11,11-tetramethyl-26-oxo-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoate

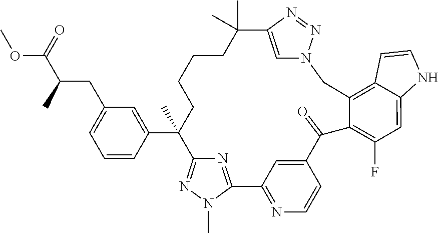 [SMILES: COC(=O)[C@H](C)Cc1cccc([C@@]2(C)CCCCC(C)(C)c3cn(nn3)Cc3c(c(F)cc4[nH]ccc34)C(=O)c3ccnc(c3)-c3nc2nn3C)c1]

Step D: To a stirred solution of Step C product (26 mg, 0.0369 mmol) in dimethylsulfoxide (3 mL) was added 1-hydroxy-1,2-benziodoxol-3(1h)-one 1-oxide (41 mg, 0.15 mmol). The mixture was stirred at room temperature for 2 hours, then diluted with ethyl acetate (20 mL). The solution was washed with saturated sodium bicarbonate, brine, dried over sodium sulfate, filtered, and concentrated. The residue was purified by automated flash chromatography (4 g silica gel column, eluting with 0-70% ethyl acetate in petroleum ether) to give the title compound (13 mg, 50%) as a solid. LC-MS: MS (ESI): 703 m/z [M+H]⁺, retention time: 1.39 minutes, purity: 43% (254 nm) (LC-MS Method 35).

Compound 139. (2R)-3-[3-[(6R)-24-Fluoro-3,6,11,11-tetramethyl-26-oxo-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-ylJ]henyl]-2-methyl-propanoic acid

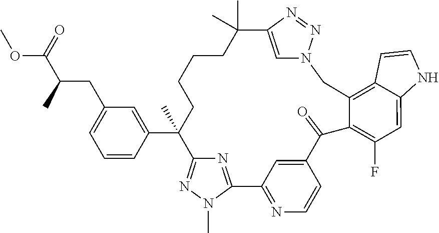 [SMILES: COC(=O)[C@H](C)Cc1cccc([C@@]2(C)CCCCC(C)(C)c3cn(nn3)Cc3c(c(F)cc4[nH]ccc34)C(=O)c3ccnc(c3)-c3nc2nn3C)c1]

Step E: To a stirred solution of Step D product (13 mg, 0.018 mmol) in tetrahydrofuran (1 mL) and water (1 mL) was added lithium hydroxide monohydrate (3.9 mg, 0.0925 mmol). The reaction mixture was stirred at room temperature overnight, acidified with 1N hydrochloric acid to pH ˜4. The mixture was diluted with ethyl acetate (20 mL), washed with brine, dried over sodium sulphate, filtered, and concentrated. The residue was purified by reverse phase prep-HPLC to give the title compound (2.6 mg, 20%) as a solid. LC-MS: MS (ESI): 689 m/z [M+H]⁺, retention time: 1.94 minutes, purity: >99% (214 nm) (LC-MS Method 26). ¹H NMR (500 MHz, CD₃OD) δ 8.94 (d, J=5.0 Hz, 1H), 7.94 (d, J=4.5 Hz, 1H), 7.83 (s, 1H), 7.54 (s, 1H), 7.49 (d, J=3.5 Hz, 1H), 7.19 (d, J=11.0 Hz, 1H), 7.09 (t, J=8.0 Hz, 1H), 7.02 (s, 1H), 6.99 (d, J=3.0 Hz, 1H), 6.95 (d, J=8.0 Hz, 2H), 6.26 (d, J=14.5 Hz, 1H), 5.84 (d, J=14.5 Hz, 1H), 4.35 (s, 3H), 2.94-2.85 (m, 1H), 2.62-2.50 (m, 2H), 2.04-1.97 (m, 1H), 1.95-1.85 (m, 1H), 1.62-1.45 (m, 4H), 1.44-1.24 (m, 3H), 1.13 (s, 3H), 1.07 (s, 3H), 1.03 (d, J=6.5 Hz, 3H), 0.63-0.42 (m, 2H) ppm.

Example 140. Compound 140. 3-[3-[(6R)-24-Fluoro-3,6,11,11-tetramethyl-26-oxo-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]propanoic acid

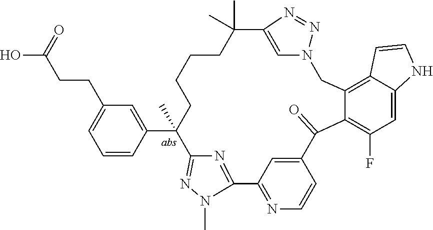 [SMILES: Cn1nc2nc1-c1cc(ccn1)C(=O)c1c(F)cc3[nH]ccc3c1Cn1cc(nn1)C(C)(C)CCCC[C@]2(C)c1cccc(CCC(=O)O)c1]

(6R)-24-Fluoro-6-(3-iodophenyl)-3,6,11,11-tetramethyl-26-tetrahydropyran-2-yloxy-3,4,13,14,15,21,30,33-octazahexacyclo[25.3.1.12,5.112,15.017,25.018,22]tritriaconta-1(30),2(33),4,12(32),13,17,19,22,24,27(31),28-undecaene

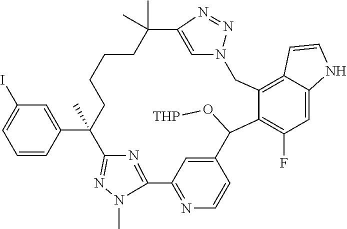

Step A: To a stirred solution of (6R)-24-fluoro-6-(3-iodophenyl)-3,6,11,11-tetramethyl-21-(p-tolylsulfonyl)-26-tetrahydropyran-2-yloxy-3,4,13,14,15,21,30,33-octazahexacyclo[25.3.1.12,5.112,15.017,25.018,22]tritriaconta-1(30),2(33),4,12(32),13,17,19,22,24,27(31),28-undecaene (Step A product of Example 139, 150 mg, 0.15 mmol) in tetrahydrofuran (1 mL) was added tetra-n-butylammonium fluoride (1M on tetrahydrofuran) (1.5 mL, 1.55 mmol). The reaction mixture was stirred at room temperature for 1 hour and diluted with ethyl acetate (20 mL). The mixture was washed with water, dried over sodium sulfate, filtered, and concentrated. The residue was purified by automated flash chromatography (10 g silica gel column, eluting with 0-70% ethyl acetate in petroleum ether) to afford the title compound (110 mg, 87%) as a mixture of four diastereomers. LC-MS: MS (ESI): 815 m/z [M+H]⁺, retention time: 2.32+2.34+2.39 minutes, purity: 21+46+22% (254 nm) (LC-MS Method 8).

Ethyl (E)-3-[3-[(6R)-24-fluoro-3,6,11,11-tetramethyl-26-tetrahydropyran-2-yloxy-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]prop-2-enoate

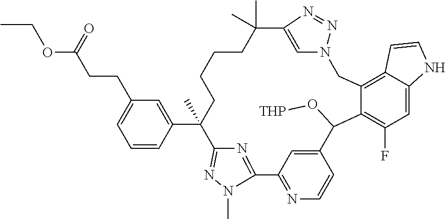

Step B: To a stirred solution of Step A product (110 mg, 0.14 mmol) in 1,4-dioxane/water (10:1) (10 mL) was added ethyl (E)-3-(4,4,5,5-tetramethyl-1,3,2-dioxaborolan-2-yl)acrylate (61 mg, 0.27 mmol), potassium carbonate (0.056 g, 0.41 mmol) and [1,1′-bis(diphenylphosphino)ferrocene]dichloropalladium(II) (0.012 g, 0.014 mmol). The mixture was purged with nitrogen and stirred at 90° C. for 4 hours, cooled to room temperature, and diluted with ethyl acetate (20 mL). The solution was washed with brine, dried over sodium sulfate, filtered, and concentrated. The residue was purified by automated flash chromatography (10 g silica gel column, eluting with 0-100% ethyl acetate in petroleum ether) to give the title compound (90 mg, 84%) as an inseparable diastereomers. LC-MS: MS (ESI): 787 m/z [M+H]⁺, retention time: 2.28+2.31 minutes, purity: 43+38% (254 nm) (LC-MS Method 8).

Ethyl 3-[3-[(6R)-24-fluoro-3,6,11,11-tetramethyl-26-tetrahydropyran-2-yloxy-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]propanoate

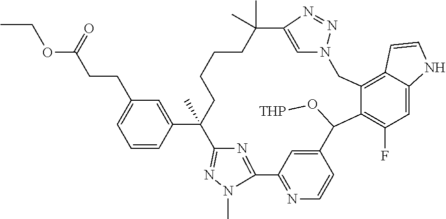

Step C: To a stirred solution of Step B product (90 mg, 0.11 mmol) in tetrahydrofuran (10 mL) was added 10% palladium on carbon (wetted with ca. 55% Water, 10%, 45 mg). The reaction mixture was stirred under hydrogen at 50° C. for 2 hours, then filtered through a pad of Celite. The filter cake was washed with ethanol (3×10 mL). The filtrate was concentrated to give the title compound (90 mg, 99%) as a solid of four diastereomer mixture. LC-MS: MS (ESI): 789 m/z [M+H]⁺, retention time: 1.48+1.49 minutes, purity: 27+39% (254 nm) (LC-MS Method 35).

Compound 140. 3-[3-[(6R)-24-Fluoro-3,6,11,11-tetramethyl-26-oxo-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]propanoic acid

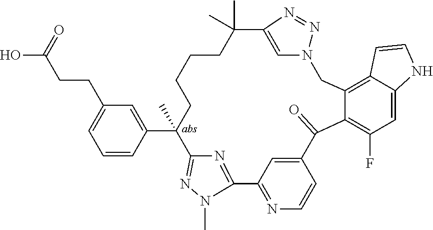 [SMILES: Cn1nc2nc1-c1cc(ccn1)C(=O)c1c(F)cc3[nH]ccc3c1Cn1cc(nn1)C(C)(C)CCCC[C@]2(C)c1cccc(CCC(=O)O)c1]

Step D: Exchanging Methyl (2R)-3-[3-[(6R)-24-fluoro-3,6,11,11-tetramethyl-26-tetrahydropyran-2-yloxy-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoate (Step B product of Example 139) with Step C product (90 mg, 0.11 mmol), the reaction procedure sequence (Steps C, E and D, in this order) described for Example 139 was used to prepare the title compound (23 mg, 40%) as a white solid. LC-MS: MS (ESI): 675 m/z [M+H]⁺, retention time: 1.43 minutes, purity: >99% (214 nm) (LC-MS Method 35). ¹H NMR (500 MHz, CD₃OD) δ 8.94 (d, J=5.0 Hz, 1H), 7.94 (d, J=5.0 Hz, 1H), 7.82 (s, 1H), 7.54 (s, 1H), 7.48 (d, J=2.5 Hz, 1H), 7.17 (d, J=10.5 Hz, 1H), 7.09 (t, J=7.5 Hz, 1H), 7.05 (s, 1H), 7.00-6.90 (m, 3H), 6.25 (d, J=14.5 Hz, 1H), 5.84 (d, J=14.5 Hz, 1H), 4.36 (s, 3H), 2.81 (t, J=6.4 Hz, 2H), 2.49 (t, J=6.4 Hz, 2H), 2.06-1.86 (m, 2H), 1.62-1.42 (m, 4H), 1.40-1.20 (m, 2H), 1.13-1.02 (m, 7H), 0.60-0.41 (m, 2H) ppm.

Example 141. Compound 141. (2S)-3-[3-[(6R)-24-fluoro-3,6,11,11-tetramethyl-26-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]-2-methyl-propanoic acid

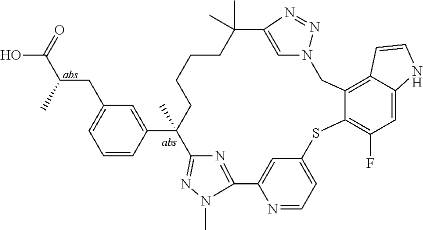 [SMILES: C[C@@H](Cc1cccc([C@@]2(C)CCCCC(C)(C)c3cn(nn3)Cc3c(c(F)cc4[nH]ccc34)Sc3ccnc(c3)-c3nc2nn3C)c1)C(=O)O]

24-Fluoro-6-(3-iodophenyl)-3,6,11,11-tetramethyl-21-(p-tolylsulfonyl)-26-thia-3,4,13,14,15,21,30,33-octazahexacyclo[25.3.1.12,5.112,15.017,25.018,22]tritriaconta-1(31),2(33),4,12(32),13,17,19,22,24,27,29-undecaene

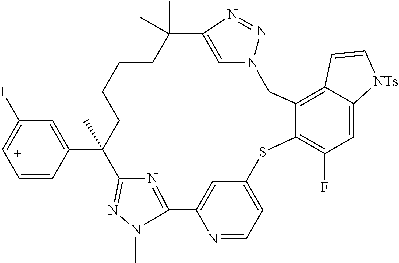 [SMILES: Cc1ccc(S(=O)(=O)n2ccc3c4c(c(F)cc32)Sc2ccnc(c2)-c2nc(nn2C)[C@@](C)(C2=CC(I)=[C+]C=C2)CCCCC(C)(C)c2cn(nn2)C4)cc1]

Step A. Exchanging (6-fluoro-5-(4-fluoro-3-(imino(methylthio)methyl)phenoxy)-1-(phenylsulfonyl)-1H-indol-4-yl)methyl acetate hydroiodide (Intermediate 82-3) with methyl 4-((6-fluoro-4-(hydroxymethyl)-1-tosyl-1H-indol-5-yl)thio)pyridine-2-carbimidothioate (Intermediate 113-1, 1.93 g, 3.85 mmol), and 2-(3-bromophenyl)-N′,2-dimethyl-5-((2-methylbut-3-yn-2-yl)oxy)pentanehydrazide (Intermediate 83-4) with (R)-2-(3-iodophenyl)-N′,2,7,7-tetramethylnon-8-ynehydrazide (Intermediate 83-8, 2.50 g, 5.86 mmol), the reaction procedure sequence (Steps A and C described for Example 97A and 97B, followed by Step B described for Example 2) was followed to prepare the title compound (500 mg, 0.564 mmol) as a white solid. LC-MS: MS (ESI): 887 m/z [M+H]⁺, retention time: 2.64 minutes, purity: 81% (214 nm) (LC-MS Method 2).

Compound 141. (2S)-3-[3-[(6R)-24-fluoro-3,6,11,11-tetramethyl-26-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]-2-meth 1-propanoic acid

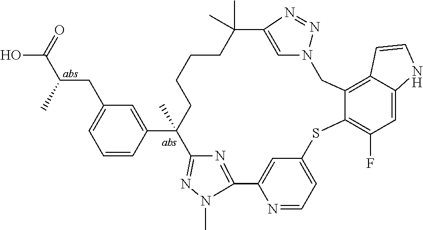 [SMILES: C[C@@H](Cc1cccc([C@@]2(C)CCCCC(C)(C)c3cn(nn3)Cc3c(c(F)cc4[nH]ccc34)Sc3ccnc(c3)-c3nc2nn3C)c1)C(=O)O]

Step B: Exchanging (6R)-24-fluoro-6-(3-iodophenyl)-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaene (Step A product of Example 138) with 24-Fluoro-6-(3-iodophenyl)-3,6,11,11-tetramethyl-21-(p-tolylsulfonyl)-26-thia-3,4,13,14,15,21,30,33-octazahexacyclo[25.3.1.12,5.112,15.017,25.018,22]tritriaconta-1(31),2(33),4,12(32),13,17,19,22,24,27,29-undecaene (Step A product of this Example, 250 mg, 0.282 mmol), the reaction procedure sequence (Steps B and C) described for Example 138 was followed to prepare the title compound (5.8 mg, 0.00837 mmol) as a white solid. LC-MS: MS (ESI): 693 m/z [M+H]⁺, retention time: 1.99 minutes, purity: >99% (214 nm) (LC-MS Method 26). ¹H NMR (500 MHz, CD₃OD) δ 8.50 (d, J=5.5 Hz, 1H), 7.50 (dd, J=5.5, 1.5 Hz, 1H), 7.41-7.34 (m, 2H), 7.34-7.29 (m, 2H), 7.11 (t, J=7.5 Hz, 1H), 7.00-6.94 (m, 3H), 6.58 (d, J=3.0 Hz, 1H), 6.38 (d, J=15.0 Hz, 1H), 6.00 (d, J=15.0 Hz, 1H), 4.27 (s, 3H), 2.96-2.91 (m, 1H), 2.64-2.53 (m, 2H), 1.95-1.90 (m, 1H), 1.82-1.77 (m, 1H), 1.56 (s, 3H), 1.40-1.24 (m, 5H), 1.13 (s, 3H), 1.05 (d, J=6.5 Hz, 3H), 0.98 (s, 3H), 0.88-0.81 (m, 1H), 0.80-0.70 (m, 1H), 0.50-0.40 (m, 1H) ppm.

Example 142. Compound 142. (2S)-3-[3-[(6R)-24-fluoro-26-hydroxy-3,6,11,11-tetramethyl-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoic acid

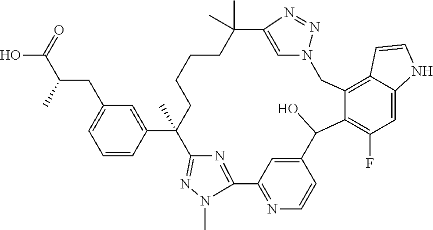 [SMILES: C[C@@H](Cc1cccc([C@@]2(C)CCCCC(C)(C)c3cn(nn3)Cc3c(c(F)cc4[nH]ccc34)C(O)c3ccnc(c3)-c3nc2nn3C)c1)C(=O)O]

Methyl (2S)-3-[3-[(6R)-24-fluoro-3,6,11,11-tetramethyl-21-(p-tolylsulfonyl)-26-tetrahydropyran-2-yloxy-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]-2-methyl-propanoate

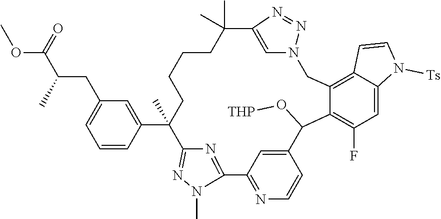

Step A: A suspension of iodine (12 mg, 0.045 mmol) and zinc (178 mg, 2.72 mmol) in tetrahydrofuran (20 mL) was stirred at room temperature for 40 minutes under Argon, then treated with methyl (2R)-3-iodo-2-methyl-propanoate (154 mg, 0.675 mmol), and stirred at room temperature for 40 minutes. To this mixture was added Bis(tri-tert-butylphosphine)palladium(0) (23 mg, 0.045 mmol) and (6R)-24-fluoro-6-(3-iodophenyl)-3,6,11,11-tetramethyl-21-(p-tolylsulfonyl)-26-tetrahydropyran-2-yloxy-3,4,13,14,15,21,30,33-octazahexacyclo[25.3.1.12,5.112,15.017,25.018,22]tritriaconta-1(30),2(33),4,12(32),13,17,19,22,24,27(31),28-undecaene (Step A product of Example 139, 440 mg, 0.45 mmol). The reaction mixture was stirred at 60° C. for 5 hours, cooled to room temperature, quenched with water (100 mL), and extracted with ethyl acetate (3×50 mL). The combined organic layers were washed with brine (30 mL), dried over sodium sulfate, filtered, and concentrated. The residue was purified by automated flash chromatography (10 g silica gel column, eluting with 0-80% ethyl acetate in petroleum ether for 60 min) to give the title compound (310 mg, 72%) as a yellow solid. LC-MS: MS (ESI): 943 m/z [M+H]⁺, retention time: 2.41+2.44+2.48 minutes, purity: 27%, 51%, and 21%, respectively (214 nm, mixture of 4 diastereomers) (LC-MS Method 8).

Methyl (2S)-3-[3-[(6R)-24-fluoro-26-hydroxy-3,6,11,11-tetramethyl-21-(p-tolylsulfonyl)-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoate

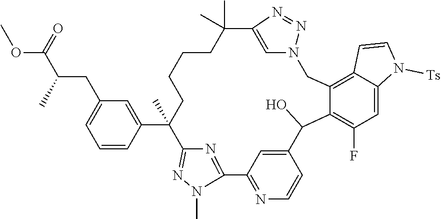 [SMILES: COC(=O)[C@@H](C)Cc1cccc([C@@]2(C)CCCCC(C)(C)c3cn(nn3)Cc3c(c(F)cc4c3ccn4S(=O)(=O)c3ccc(C)cc3)C(O)c3ccnc(c3)-c3nc2nn3C)c1]

Step B: To a stirred solution of Step A product (310 mg, 0.329 mmol) in methanol (5 mL) was added pyridinium p-toluenesulfonate (330 mg, 1.31 mmol). The reaction mixture was stirred at 60° C. for 5 hours, diluted with ethyl acetate (50 mL), washed with water, brine, dried over sodium sulfate, filtered, and concentrated. The residue was purified by automated flash chromatography (10 g silica gel column, eluting with 0-100% ethyl acetate in petroleum ether) to give the title compound (250 mg, 88%) as a solid. LC-MS: MS (ESI): 859 m/z [M+H]⁺, retention time: 2.22+2.26 minutes, purity: 43+57% (214 nm, mixture of 2 diastereomers) (LC-MS Method 8).

Compound 142. (2S)-3-[3-[(6R)-24-fluoro-26-hydroxy-3,6,11,11-tetramethyl-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoic acid (Two Diastereomeric Mixture)

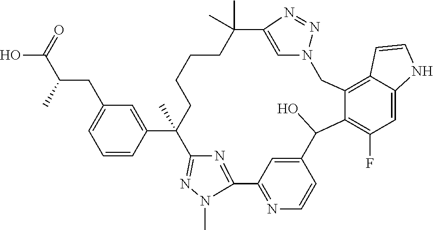 [SMILES: C[C@@H](Cc1cccc([C@@]2(C)CCCCC(C)(C)c3cn(nn3)Cc3c(c(F)cc4[nH]ccc34)C(O)c3ccnc(c3)-c3nc2nn3C)c1)C(=O)O]

Step C: To a stirred solution of Step B product (230 mg, 0.27 mmol) in tetrahydrofuran (1 mL) and water (1 mL) was added lithium hydroxide monohydrate (56 mg, 1.34 mmol). The reaction was stirred at room temperature overnight, then acidified with 1N hydrochloric acid to pH ˜4, and diluted with ethyl acetate (50 mL). The mixture was washed with brine, dried over sodium sulfate, filtered, and concentrated to give a crude product (158 mg, 85%) as a solid. A portion of this crude product (36 mg) was subject to prep-HPLC purification to give the title compound (20 mg, 55%) as a white solid. LC-MS: MS (ESI): 691 m/z [M+H]⁺, retention time: 1.30 minutes, purity: 99% (214 nm, mixture of 2 diastereomers) (LC-MS Method 35).

Example 143. Compound 143. (2S)-3-[3-[(6R)-24-fluoro-3,6,11,11-tetramethyl-26-oxo-3,4,13,14,15,21,30,33-octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoic acid

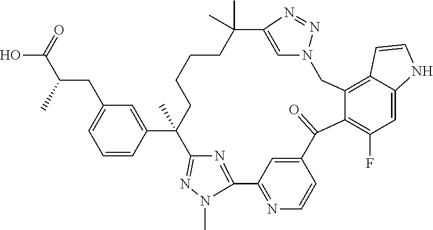 [SMILES: C[C@@H](Cc1cccc([C@@]2(C)CCCCC(C)(C)c3cn(nn3)Cc3c(c(F)cc4[nH]ccc34)C(=O)c3ccnc(c3)-c3nc2nn3C)c1)C(=O)O]

To a stirred solution of crude (2S)-3-[3-[(6R)-24-fluoro-26-hydroxy-3,6,11,11-tetramethyl-3,4,13,14,15,21,30,33-Octazahexacyclo[25.3.1.12,5.112,15.017,25.018,22]tritriaconta-1(30),2(33),4,12(32),13,17,19,22,24,27(31),28-undecaen-6-yl]phenyl]-2-methyl-propanoic acid (Example 142, 120 mg, 0.174 mmol) was added 1-hydroxy-1,2-benziodoxol-3(1h)-one 1-oxide (150 mg, 0.521 mmol) in dimethylsulfoxide (5 mL). The reaction was stirred at room temperature for 3 hours, then diluted with ethyl acetate (30 mL). The mixture was washed with saturated sodium bicarbonate, brine, dried over sodium sulfate, filtered, and concentrated. The residue was purified by prep-HPLC to give the title compound (69 mg, 57%) as a white solid. LC-MS: MS (ESI): 689 m/z [M+H]⁺, retention time: 1.43 minutes, purity: >99% (214 nm) (LC-MS Method 35). ¹H NMR (500 MHz, CD₃OD) δ 8.94 (d, J=5.0 Hz, 1H), 7.94 (d, J=5.0 Hz, 1H), 7.82 (s, 1H), 7.54 (s, 1H), 7.48 (d, J=2.5 Hz, 1H), 7.17 (d, J=10.5 Hz, 1H), 7.09 (t, J=7.5 Hz, 1H), 7.02-6.92 (m, 4H), 6.25 (d, J=14.5 Hz, 1H), 5.84 (d, J=14.5 Hz, 1H), 4.36 (s, 3H), 2.92-2.88 (m, 1H), 2.63-2.52 (m, 2H), 2.03-1.86 (m, 2H), 1.64-1.52 (m, 4H), 1.40-1.20 (m, 2H), 1.13 (s, 3H), 1.09-1.05 (m, 4H), 1.03 (d, J=6.5 Hz, 3H), 0.60-0.41 (m, 2H) ppm.

Example 144. Compound 144. 3-[3-(23,24-difluoro-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl)phenyl]propanoic acid

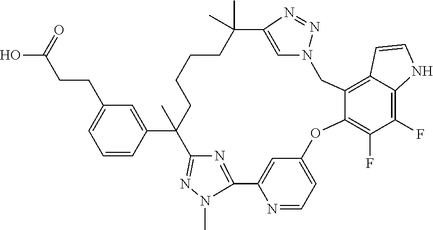 [SMILES: Cn1nc2nc1-c1cc(ccn1)Oc1c(F)c(F)c3[nH]ccc3c1Cn1cc(nn1)C(C)(C)CCCCC2(C)c1cccc(CCC(=O)O)c1]

23,24-Difluoro-6-(3-iodophenyl)-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaene

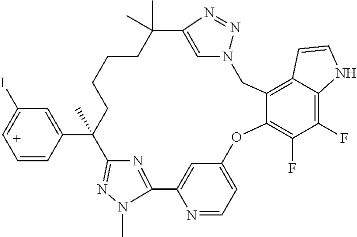 [SMILES: Cn1nc2nc1-c1cc(ccn1)Oc1c(F)c(F)c3[nH]ccc3c1Cn1cc(nn1)C(C)(C)CCCC[C@]2(C)C1=CC(I)=[C+]C=C1]

Step A: Exchanging 2-(3-bromophenyl)-5-(but-2-yn-1-yloxy)-N′,2-dimethylpentanehydrazide (Intermediate 83-1) with (R)-2-(3-iodophenyl)-N′,2,7,7-tetramethylnon-8-ynehydrazide (Intermediate 83-8, 1.74 g, 4.08 mmol), and 2-fluoro-5-((6-fluoro-4-vinyl-1H-indol-5-yl)oxy)benzimidothioate hydroiodide (Intermediate 82-1) with methyl 4-((6,7-difluoro-4-(hydroxymethyl)-1H-indol-5-yl)oxy)pyridine-2-carbimidothioate (Intermediate 82-12, 1.50 g, 4.29 mmol), the reaction procedure sequence (Steps A and E as described in Example 93A, 93B, followed by Steps B as described in Example 2, in this order) was used to prepare the title compound (0.72 g, 0.980 mmol) as a white solid. LC-MS: MS (ESI): 735 m/z [M+H]⁺, retention time: 2.19 minutes, purity: 82% (214 nm) (LC-MS Method 2).

Compound 144. 3-[3-(23,24-difluoro-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl)phenyl]propanoic acid

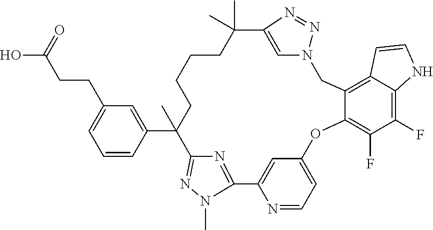 [SMILES: Cn1nc2nc1-c1cc(ccn1)Oc1c(F)c(F)c3[nH]ccc3c1Cn1cc(nn1)C(C)(C)CCCCC2(C)c1cccc(CCC(=O)O)c1]

Step B: Exchanging 23,29-difluoro-6-(3-iodophenyl)-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaene (Step B product of Example 2) with 23,24-Difluoro-6-(3-iodophenyl)-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaene (Step A product of this Example, 120 mg, 0.163 mmol), the reaction procedure sequence (Steps C, D and E) described for Example 2 was followed to prepare the title compound (24 mg, 0.0353 mmol) as a white solid. LC-MS: MS (ESI): 681 m/z [M+H]⁺, retention time: 2.04 minutes, purity: >99% (214 nm) (LC-MS Method 36). ¹H NMR (400 MHz, CD₃OD) δ 8.66 (d, J=5.6 Hz, 1H), 7.47 (d, J=3.2 Hz, 1H), 7.41-7.32 (m, 2H), 7.14 (d, J=2.4 Hz, 1H), 7.10 (t, J=7.6 Hz, 1H), 7.03 (s, 1H), 6.98 (d, J=7.6 Hz, 1H), 6.92 (d, J=7.6 Hz, 1H), 6.83 (t, J=3.2 Hz, 1H), 5.95 (d, J=14.4 Hz, 1H), 5.72 (d, J=14.4 Hz, 1H), 4.36 (s, 3H), 2.80 (t, J=7.6 Hz, 2H), 2.49 (t, J=7.6 Hz, 2H), 2.05-1.98 (m, 2H), 1.55 (s, 3H), 1.49-1.42 (m, 2H), 1.25-1.16 (m, 2H), 1.11 (s, 3H), 1.04 (s, 3H), 0.62-0.51 (m, 2H) ppm.

Example 145. Compound 145. (2R)-3-[3-(23,24-difluoro-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl)phenyl]-2-methyl-propanoic acid

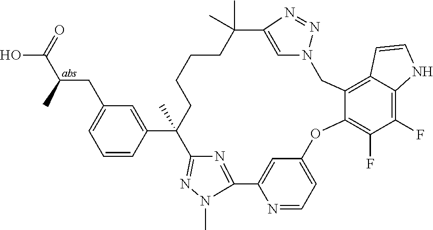 [SMILES: C[C@H](Cc1cccc([C@@]2(C)CCCCC(C)(C)c3cn(nn3)Cc3c(c(F)c(F)c4[nH]ccc34)Oc3ccnc(c3)-c3nc2nn3C)c1)C(=O)O]

Exchanging (6R)-24-fluoro-6-(3-iodophenyl)-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaene (Step A product of Example 138) with 23,24-Difluoro-6-(3-iodophenyl)-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaene (Step A product of Example 144, 150 mg, 0.204 mmol), and methyl (2R)-3-iodo-2-methyl-propanoate with methyl (2S)-3-iodo-2-methyl-propanoate (140 mg, 0.613 mmol), the reaction procedure sequence (Steps B and C) described for Example 138 was followed to prepare the title compound (12 mg, 0.017 mmol) as a white solid. LC-MS: MS (ESI): 695 m/z [M+H]⁺, retention time: 2.08 minutes, purity: >99% (214 nm) (LC-MS Method 36). ¹H NMR (400 MHz, CD₃OD) δ 8.66 (d, J=5.6 Hz, 1H), 7.46 (d, J=3.2 Hz, 1H), 7.42-7.33 (m, 2H), 7.16 (d, J=2.4 Hz, 1H), 7.10 (t, J=7.6 Hz, 1H), 6.99-6.92 (m, 3H), 6.83 (t, J=3.2 Hz, 1H), 5.94 (d, J=14.4 Hz, 1H), 5.72 (d, J=14.4 Hz, 1H), 4.36 (s, 3H), 2.93-2.85 (m, 1H), 2.60-2.52 (m, 2H), 2.07-1.97 (m, 2H), 1.54 (s, 3H), 1.49-1.39 (m, 2H), 1.24-1.16 (m, 1H), 1.11 (s, 3H), 1.05-1.03 (m, 6H), 0.91-0.84 (m, 1H), 0.63-0.52 (m, 2H) ppm.

Example 146. Compound 146. (2S)-3-[3-[(6R)-23,24-difluoro-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]-2-methyl-propanoic acid

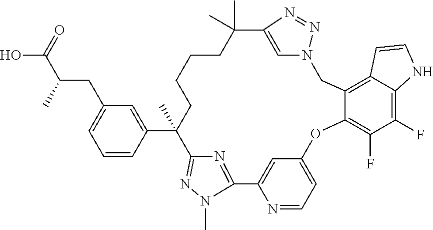 [SMILES: C[C@@H](Cc1cccc([C@@]2(C)CCCCC(C)(C)c3cn(nn3)Cc3c(c(F)c(F)c4[nH]ccc34)Oc3ccnc(c3)-c3nc2nn3C)c1)C(=O)O]

Exchanging (6R)-24-fluoro-6-(3-iodophenyl)-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaene (Step A product of Example 138) with 23,24-Difluoro-6-(3-iodophenyl)-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaene (Step A product of Example 144, 150 mg, 0.204 mmol), the reaction procedure sequence (Steps B and C) described for Example 138 was followed to prepare the title compound (33 mg, 0.0475 mmol) as a white solid. LC-MS: MS (ESI): 695 m/z [M+H]⁺, retention time: 1.61 minutes, purity: >99% (214 nm) (LC-MS Method 26). ¹H NMR (400 MHz, CD₃OD) δ 8.66 (d, J=5.6 Hz, 1H), 7.46 (d, J=3.2 Hz, 1H), 7.42-7.33 (m, 2H), 7.16 (d, J=2.4 Hz, 1H), 7.10 (t, J=7.6 Hz, 1H), 6.99-6.92 (m, 3H), 6.83 (t, J=3.2 Hz, 1H), 5.94 (d, J=14.4 Hz, 1H), 5.72 (d, J=14.4 Hz, 1H), 4.36 (s, 3H), 2.93-2.85 (m, 1H), 2.58-2.52 (m, 2H), 2.07-1.96 (m, 2H), 1.54 (s, 3H), 1.49-1.39 (m, 2H), 1.24-1.16 (m, 1H), 1.11 (s, 3H), 1.05-1.03 (m, 6H), 0.91-0.84 (m, 1H), 0.63-0.52 (m, 2H) ppm.

Example 147. Compound 147. (2R)-3-[3-[(6R)-24-fluoro-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]-2-methyl-propanoic acid

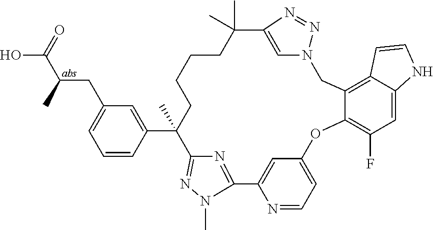 [SMILES: C[C@H](Cc1cccc([C@@]2(C)CCCCC(C)(C)c3cn(nn3)Cc3c(c(F)cc4[nH]ccc34)Oc3ccnc(c3)-c3nc2nn3C)c1)C(=O)O]

and

Example 148. Compound 148. 24-Fluoro-3,6,11,11-tetramethyl-6-phenyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaene

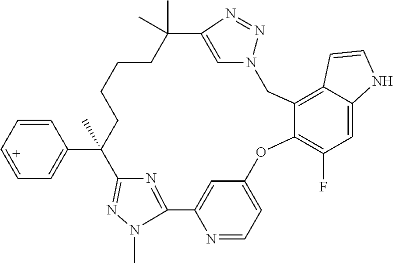 [SMILES: Cn1nc2nc1-c1cc(ccn1)Oc1c(F)cc3[nH]ccc3c1Cn1cc(nn1)C(C)(C)CCCC[C@]2(C)C1=CC=[C+]C=C1]

Exchanging methyl (2R)-3-iodo-2-methyl-propanoate with methyl (2S)-3-iodo-2-methyl-propanoate (1.50 eq, 0.98 g, 4.31 mmol), the reaction procedure sequence (Step B and C) described for Example 138 was followed to prepare Compound 147 (927 mg, 1.37 mmol) as a white solid. LC-MS: MS (ESI): 677 m/z [M+H]⁺, retention time: 2.16 minutes, purity: >99% (214 nm) (LC-MS Method 37). ¹H NMR (500 MHz, CD₃OD) δ 8.62 (d, J=6.0 Hz, 1H), 7.41-7.39 (m, 2H), 7.35-7.31 (m, 2H), 7.15 (s, 1H), 7.09 (t, J=8.0 Hz, 1H), 7.00 (s, 1H), 6.95-6.92 (m, 2H), 6.75 (d, J=2.5 Hz, 1H), 5.98 (d, J=14.5 Hz, 1H), 5.75 (d, J=14.5 Hz, 1H), 4.34 (s, 3H), 2.91-2.88 (m, 1H), 2.60-2.53 (m, 2H), 2.05-1.93 (m, 2H), 1.55 (s, 3H), 1.50-1.38 (m, 2H), 1.25-1.18 (m, 1H), 1.12 (s, 3H), 1.05-1.03 (m, 7H), 0.64-0.56 (m, 2H) ppm.

Along with this major product, a by-product Compound 148 (55 mg, 0.0923 mmol, originated from de-iodination product in corresponding Step B of Example 138) was also separated at this last step. LC-MS: MS (ESI): 591 m/z [M+H]⁺, retention time: 1.94 minutes, purity: >99% (214 nm) (LC-MS Method 38). ¹H NMR (500 MHz, CD₃OD) δ 8.62 (d, J=6.0 Hz, 1H), 7.39 (s, 2H), 7.34-7.32 (m, 1H), 7.28 (d, J=10.5 Hz, 1H), 7.18-7.06 (m, 6H), 6.75 (d, J=3.0 Hz, 1H), 5.98 (d, J=14.5 Hz, 1H), 5.74 (d, J=14.5 Hz, 1H), 4.34 (s, 3H), 2.05-1.95 (m, 2H), 1.55 (s, 3H), 1.50-1.38 (m, 2H), 1.25-1.18 (m, 1H), 1.12 (s, 3H), 1.06-1.00 (m, 4H), 0.66-0.54 (m, 2H) ppm.

Example 149. Compound 149. 3-[3-[(6R)-24-fluoro-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]-2,2-dimethyl-propanoic acid

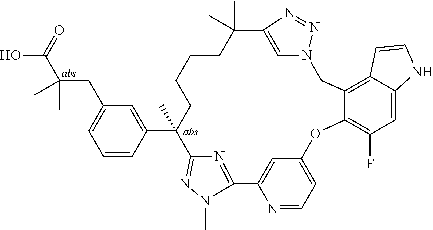 [SMILES: Cn1nc2nc1-c1cc(ccn1)Oc1c(F)cc3[nH]ccc3c1Cn1cc(nn1)C(C)(C)CCCC[C@]2(C)c1cccc(CC(C)(C)C(=O)O)c1]

Exchanging methyl (2R)-3-iodo-2-methyl-propanoate with methyl 3-iodo-2,2-dimethyl-propanoate (1.5 eq, 66 mg, 0.272 mmol), the reaction procedure sequence (Step B and C) described for Example 138 was followed to prepare the title compound (34 mg, 0.0498 mmol) as a white solid. LC-MS: MS (ESI): 691 m/z [M+H]⁺, retention time: 1.99 minutes, purity: >99% (214 nm) (LC-MS Method 28). ¹H NMR (400 MHz, CD₃OD) δ 8.64 (d, J=5.6 Hz, 1H), 7.48 (s, 1H), 7.41 (d, J=3.2 Hz, 1H), 7.37-7.31 (m, 2H), 7.14 (s, 1H), 7.08 (t, J=7.6 Hz, 1H), 6.97-6.91 (m, 3H), 6.76 (d, J=3.2 Hz, 1H), 5.99 (d, J=14.4 Hz, 1H), 5.77 (d, J=14.4 Hz, 1H), 4.35 (s, 3H), 2.80-2.60 (m, 2H), 2.03-1.95 (m, 2H), 1.55 (s, 3H), 1.48-1.42 (m, 2H), 1.25-1.19 (m, 2H), 1.13 (s, 3H), 1.07-1.03 (m, 9H), 0.64-0.55 (m, 2H) ppm.

Example 150. Compound 150. 3-[3-[(6R)-24-fluoro-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]propanoic acid

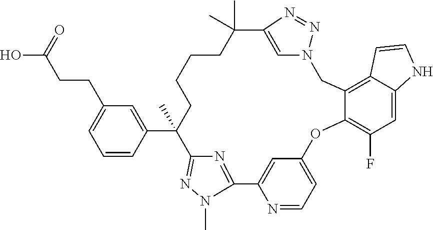 [SMILES: Cn1nc2nc1-c1cc(ccn1)Oc1c(F)cc3[nH]ccc3c1Cn1cc(nn1)C(C)(C)CCCC[C@]2(C)c1cccc(CCC(=O)O)c1]

Exchanging 23,29-difluoro-6-(3-iodophenyl)-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaene (Step A product of Example 2) with (6R)-24-Fluoro-6-(3-iodophenyl)-3,6,11,11-tetramethyl-26-oxa-3,4,13,14,15,21,30,33-octazahexacyclo[25.3.1.12,5.112,15.017,25.018,22]tritriaconta-1(31),2(33),4,12(32),13,17,19,22,24,27,29-undecaene (Step A product of Example 138, 130 mg, 0.181 mmol), the reaction procedure sequence (Steps C, D and E) described for Example 704 was followed to prepare the title compound (64 mg, 0.0958 mmol) as a white solid. LC-MS: MS (ESI): 663 m/z [M+H]⁺, retention time: 1.91 minutes, purity: 95% (214 nm) (LC-MS Method 34). ¹H NMR (400 MHz, CD₃OD) δ 8.62 (d, J=5.6 Hz, 1H), 7.41-7.39 (m, 2H), 7.35-7.31 (m, 2H), 7.14-7.08 (m, 2H), 7.03 (s, 1H), 6.98-6.92 (m, 2H), 6.75 (d, J=3.2 Hz, 1H), 5.98 (d, J=14.4 Hz, 1H), 5.74 (d, J=14.4 Hz, 1H), 4.34 (s, 3H), 2.80 (t, J=7.6 Hz, 2H), 2.49 (t, J=7.6 Hz, 2H), 2.03-1.95 (m, 2H), 1.55 (s, 3H), 1.47-1.40 (m, 2H), 1.23-1.19 (m, 2H), 1.12 (s, 3H), 1.05 (s, 3H), 0.64-0.56 (m, 2H) ppm.

Example 151. Compound 151. 3-[3-[(6R)-24-fluoro-3,6,11,11-tetramethyl-26-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]propanoic acid

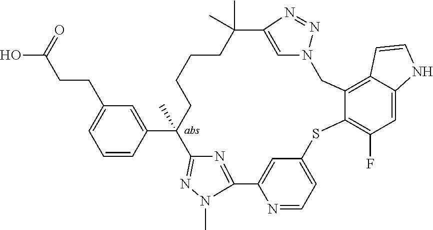 [SMILES: Cn1nc2nc1-c1cc(ccn1)Sc1c(F)cc3[nH]ccc3c1Cn1cc(nn1)C(C)(C)CCCC[C@]2(C)c1cccc(CCC(=O)O)c1]

Ethyl 3-[3-[(6R)-24-Fluoro-3,6,11,11-tetramethyl-26-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]propanoate

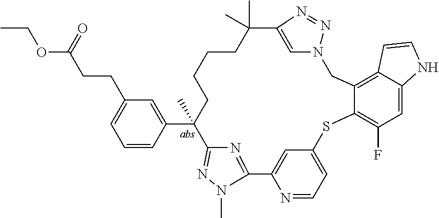 [SMILES: CCOC(=O)CCc1cccc([C@@]2(C)CCCCC(C)(C)c3cn(nn3)Cc3c(c(F)cc4[nH]ccc34)Sc3ccnc(c3)-c3nc2nn3C)c1]

Step A: Exchanging 23,29-difluoro-6-(3-iodophenyl)-6-methyl-25-oxa-3,12,13,14,20,32-hexazahexacyclo-[24.3.1.12,5.111,14.016,24.017,21]dotriaconta-1(30),2,4,11(31),12,16,18,21,23,26,28-undecaene (Step B product of Example 704) with 24-Fluoro-6-(3-iodophenyl)-3,6,11,11-tetramethyl-21-(p-tolylsulfonyl)-26-thia-3,4,13,14,15,21,30,33-octazahexacyclo[25.3.1.12,5.112,15.017,25.018,22]tritriaconta-1(31),2(33),4,12(32),13,17,19,22,24,27,29-undecaene (Step A product of Example 141, 270 mg, 0.369 mmol), the reaction procedure sequence (Steps C, D) described for Example 141 was followed to prepare the title compound (110 mg, 0.156 mmol) as a white solid. LC-MS: MS (ESI): 707 m/z [M+H]⁺, retention time: 2.16 minutes, purity: 99% (214 nm) (LC-MS Method 26).

Compound 151. 3-[3-[(6R)-24-fluoro-3,6,11,11-tetramethyl-26-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]propanoic acid

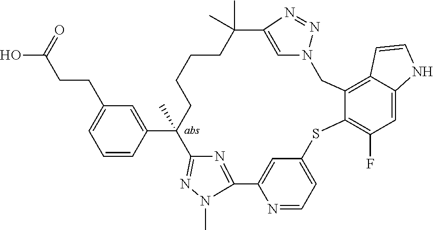 [SMILES: Cn1nc2nc1-c1cc(ccn1)Sc1c(F)cc3[nH]ccc3c1Cn1cc(nn1)C(C)(C)CCCC[C@]2(C)c1cccc(CCC(=O)O)c1]

Step B: To a stirred solution of Step A product (35 mg, 0.0495 mmol) in tetrahydrofuran (1.5 mL) and methanol (0.5 mL) was added lithium hydroxide monohydrate (11 mg, 0.25 mmol) in water (0.5 mL). The reaction was stirred at room temperature for 16 hours, diluted with water (20 mL), acidified with 1 N hydrochloric acid to pH ˜6, and extracted with ethyl acetate (3×20 mL). The combined organic layers were washed with brine, dried over sodium sulfate, filtered, and concentrated. The residue was purified by automated flash chromatography (20 g silica gel column, eluting with 0-65% ethyl acetate in petroleum ether) to give the title compound (20 mg, 0.0301 mmol) as a solid. LC-MS: MS (ESI): 679 m/z [M+H]⁺, retention time: 1.97 minutes, purity: 98% (214 nm) (LC-MS Method 26). ¹H NMR (400 MHz, CD₃OD) δ 8.49 (d, J=5.2 Hz, 1H), 7.49 (dd, J=5.2 Hz, 2.0 Hz, 1H), 7.40-7.37 (m, 2H), 7.32 (s, 1H), 7.27 (s, 1H), 7.12 (t, J=8.0 Hz, 1H), 7.03 (s, 1H), 7.00-6.92 (m, 2H), 6.56 (d, J=3.2 Hz, 1H), 6.38 (d, J=14.4 Hz, 1H), 5.98 (d, J=14.4 Hz, 1H), 4.26 (s, 3H), 2.83 (t, J=7.6 Hz, 2H), 2.52 (t, J=7.6 Hz, 2H), 1.97-1.91 (m, 1H), 1.79-1.72 (m, 1H), 1.56 (s, 3H), 1.33-1.29 (m, 3H), 1.13 (s, 3H), 0.97 (s, 3H), 0.88-0.83 (m, 1H), 0.78-0.70 (m, 1H), 0.51-0.43 (m, 1H) ppm.

Example 152. Compound 152. 3-[3-[(6R)-24-fluoro-3,6,11,11-tetramethyl-26-oxo-26λ4-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]propanoic acid

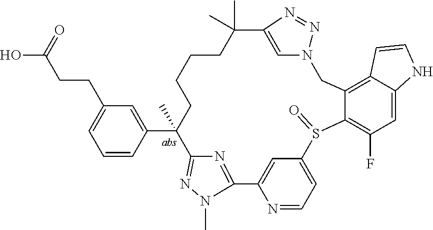 [SMILES: Cn1nc2nc1-c1cc(ccn1)S(=O)c1c(F)cc3[nH]ccc3c1Cn1cc(nn1)C(C)(C)CCCC[C@]2(C)c1cccc(CCC(=O)O)c1]

Ethyl 3-[3-[(6R)-24-fluoro-3,6,11,11-tetramethyl-21-(p-tolylsulfonyl)-26-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]propanoate

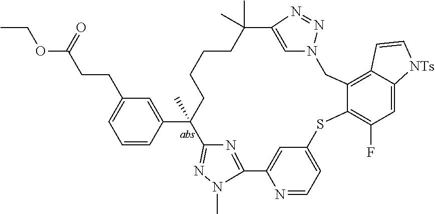 [SMILES: CCOC(=O)CCc1cccc([C@@]2(C)CCCCC(C)(C)c3cn(nn3)Cc3c(c(F)cc4c3ccn4S(=O)(=O)c3ccc(C)cc3)Sc3ccnc(c3)-c3nc2nn3C)c1]

Step A: To a stirred solution of ethyl 3-[3-[(6R)-24-fluoro-3,6,11,11-tetramethyl-26-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]propanoate (Step A product of Example 151, 70 mg, 0.0990 mmol) in acetonitrile (3 mL) were added 1-tosyl-1H-imidazole (88 mg, 0.40 mmol) and 1,8-diazabicyclo[5.4.0]undec-7-ene (61 mg, 0.40 mmol). The reaction was stirred at 30° C. for 3 days, quenched with water (10 mL), and extracted with ethyl acetate (3×20 mL). The combined organic layer was washed with water, brine, dried over sodium sulfate, filtered, and concentrated. The residue was purified by automated flash chromatography (20 g silica gel column, eluting with 0-80% ethyl acetate in petroleum ether) to give the title compound (72 mg, 0.0836 mmol, 84.00%) as a solid. LC-MS: MS (ESI): 861 m/z [M+H]⁺, retention time: 2.34 minutes, purity: 92% (214 nm) (LC-MS Method 26).

Ethyl 3-[3-[(6R)-24-fluoro-3,6,11,11-tetramethyl-26-oxo-21-(p-tolylsulfonyl)-26A4-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]propanoate

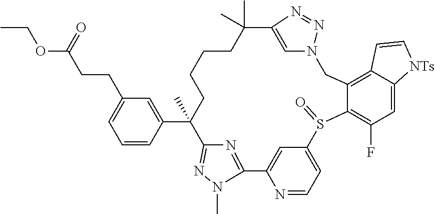 [SMILES: CCOC(=O)CCc1cccc([C@@]2(C)CCCCC(C)(C)c3cn(nn3)Cc3c(c(F)cc4c3ccn4S(=O)(=O)c3ccc(C)cc3)S(=O)c3ccnc(c3)-c3nc2nn3C)c1]

Step B: To a stirred solution of Step A product (72 mg, 0.0836 mmol) in acetonitrile (2 mL) was added (S,S)-hydrobenzoin (0.090 g, 0.418 mmol), titanium(iv) isopropoxide (0.074 mL, 0.251 mmol) and tert-butyl hydroperoxide (0.023 g, 0.251 mmol). The reaction was stirred at 30° C. for 16 hours, poured into 20 mL of water and extracted with ethyl acetate (3×15 mL). The combined organic phases were washed with brine, dried over sodium sulfate, filtered, and concentrated. The residue was purified by automated flash chromatography (20 g silica gel column, eluting with 0-90% ethyl acetate in petroleum ether) to give the title compound (55 mg, 0.0627 mmol, 75%) as a solid. LC-MS: MS (ESI): 877 m/z [M+H]⁺, retention time: 2.17 minutes, purity: 97% (214 nm) (LC-MS Method 26).

Ethyl 3-[3-[(6R)-24-fluoro-3,6,11,11-tetramethyl-26-oxo-26A4-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]propanoate

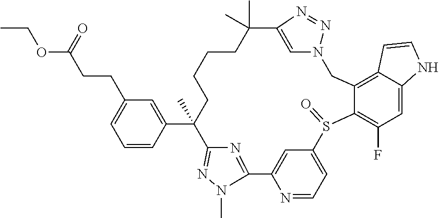 [SMILES: CCOC(=O)CCc1cccc([C@@]2(C)CCCCC(C)(C)c3cn(nn3)Cc3c(c(F)cc4[nH]ccc34)S(=O)c3ccnc(c3)-c3nc2nn3C)c1]

Step C: To a stirred solution of Step B product (55 mg, 0.0627 mmol) in tetrahydrofuran (2 mL) was added tetrabutylammonium fluoride (0.31 ml, 1.0 M in tetrahydrofuran, 0.31 mmol). The reaction was stirred at room temperature overnight, poured into 15 mL of water, and extracted with ethyl acetate (3×15 mL). The combined organic phases were washed with brine, dried over sodium sulfate, filtered, and concentrated. The residue was purified by flash chromatography (20 g silica gel column, eluting with 0-90% ethyl acetate in petroleum ether) to give the title compound (35 mg, 0.048 mmol, 77%) as a solid. LC-MS: MS (ESI): 723 m/z [M+H]⁺, retention time: 1.98 minutes, purity: 61% (214 nm) (LC-MS Method 26).

Compound 152. 3-[3-[(6R)-24-fluoro-3,6,11,11-tetramethyl-26-oxo-26A4-thia-3,4,13,14,15,21,30,33-octazahexacyclo[25.3.1.12,5.112,15.017,25.018,22]tritriaconta-1(31),2(33),4,12(32),13,17,19,22,24,27,29-undecaen-6-yl]phenyl]propanoic acid

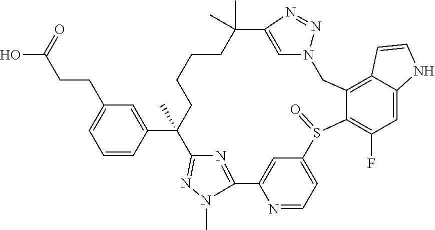 [SMILES: Cn1nc2nc1-c1cc(ccn1)S(=O)c1c(F)cc3[nH]ccc3c1Cn1cc(nn1)C(C)(C)CCCC[C@]2(C)c1cccc(CCC(=O)O)c1]

Step D: To a stirred solution of Step C product (35 mg, 0.0484 mmol) in tetrahydrofuran (1.5 mL) and methanol (0.5 mL) was added lithium hydroxide monohydrate (10 mg, 0.292 mmol) in water (0.5 mL). The reaction was stirred at room temperature for 16 hours, diluted with water (20 mL), acidified with 1 N hydrochloric acid to pH ˜6, and extracted with ethyl acetate (3×20 mL). The combined organic layers were washed with brine, dried over sodium sulfate, filtered, and concentrated. The residue was purified by prep-HPLC to give the title compound (5.0 mg, 0.00720 mmol, 15%) as a solid. LC-MS: MS (ESI): 695 m/z [M+H]⁺, retention time: 1.67 minutes, purity: 86% (214 nm) (LC-MS Method 33). ¹H NMR (500 MHz, CD₃OD) δ 9.00 (d, J=4.0 Hz, 1H), 8.19 (s, 1H), 7.97 (s, 1H), 7.50 (s, 2H), 7.25 (d, J=11.0 Hz, 1H), 7.09 (s, 1H), 7.03 (d, J=7.5 Hz, 1H), 6.93-6.91 (m, 2H), 6.83 (d, J=7.5 Hz, 1H), 6.55 (d, J=14.5 Hz, 1H), 5.98 (d, J=14.5 Hz, 1H), 4.22 (s, 3H), 2.75 (t, J=8.0 Hz, 2H), 2.44 (t, J=8.0 Hz, 2H), 1.93-1.87 (m, 1H), 1.80-1.74 (m, 1H), 1.58 (s, 3H), 1.33-1.29 (m, 4H), 1.23 (s, 3H), 1.18 (s, 3H), 0.72-0.65 (m, 1H), 0.52-0.44 (m, 1H) ppm.

### Biological Assays

Example 153. Aggregation Analysis Using Differential Static Light Scattering (DSLS)

Purified recombinant NBD1 was produced using previously described methods (A. Schmidt, J. L. Mendoza, P. J. Thomas (2011) Biochemical and Biophysical Approaches to Probe CFTR Structure (365-376) M. D. Amaral, K. Kunzelmann (eds.), Cystic Fibrosis, Methods in Molecular Biology 741, Springer Science+Business Media). The effect of test compounds on thermal stability of NBD1 was evaluated by differential static light scattering (DSLS) using the Harbinger Stargazer-384 instrument (Epiphyte Three, Toronto, Canada). Test compounds were dissolved and diluted to desired concentrations in 100% DMSO. The compounds or DMSO controls (100 nL) were stamped into wells of a 385-well low volume optical plate (Corning Inc., Corning, NY) using the Echo 555 acoustic liquid handler (Labcyte Inc., San Jose, CA).

NBD1 protein was diluted to 0.2 mg/ml in S200 buffer (50 mM Tris-HCl, 150 mM NaCl, 5 mM MgCl₂, 2 mM ATP, 2 mM DTT, pH7.6) containing 1% glycerol. 10 μL of protein solution was aliquoted into the 384-well plate harboring the test compounds and 10 uL mineral oil was overlaid onto the protein solution, using the epMotion robotic liquid handler (Eppendorf North America, Hauppauge, NY). After placing into the Stargazer instrument, the plate was heated at 1° C. per minute to 70° C. Images were captured from 25° C. to 70° C. every 0.5° C. At the end of the experiment run, instrument software integrated image files and analyzed data automatically. A linear regression curve was generated for each well, representing the increase in light scattering over time. A temperature of aggregation (T_{agg}) was calculated based on the inflection point of the curve. To better compare data across experiments the average T_{agg} for DMSO control wells was calculated and subtracted from values for wells containing compounds to obtain a “ΔT_{agg}” value. These ΔT_{agg} values reflect stabilizing efficacy of the compounds. As set forth in Table 2 below, an ΔT_{agg} value less than or equal to 5° C. is marked “A”; a value above 5° C. and less than or equal to 10° C. is marked “B”; a value greater than 10° C. and less than or equal to 25° C. is marked “C”.

Example 154. TECC24 AUC Fold Over Benchmark VX-809 @10 μM

The effects of a test agent on CFTR-mediated transepithelial chloride transport is measured using TECC24 recording analysis. Test agents are solubilized in DMSO. Solubilized test agents are mixed with incubation medium containing DMEM/F12, Ultroser G (0.5%; Crescent Chemical, catalog #67042), Hyclone Fetal Clone II (2%; GE Healthcare, catalog #SH30066.02), bovine brain extract (0.25%; Lonza, catalog #CC-4098), insulin (2.5 μg/mL), IL-13 (10 ng/mL), hydrocortisone (20 nM), transferrin (2.5 μg/mL), triiodothyronine (500 nM), ethanolamine (250 nM), epinephrine (1.5 μM), phosphoethanolamine (250 nM), and retinoic acid (10 nM). Primary human bronchial epithelial cells from a ΔF508 homozygous CF donor (CF-HBE cells; from University of North Carolina Cystic Fibrosis Tissue Procurement Center), grown on Transwell HTS 24-well cell culture inserts (Costar, catalog #3378), are exposed to test agents or controls dissolved in incubation medium. The CF-HBE cells are cultured at 36.5° C. for 48 hours before TECC24 recordings are performed in the presence or absence of test agent, a positive control or vehicle (DMSO).

Following incubation, the transwell cell culture inserts containing the test agent or control-treated CF-HBE cells are loaded onto a TECC24 apparatus (TECC v7 or MTECC v2; EP Design) to record the transepithelial voltage (VT) and resistance (TEER) using 4 AgCl electrodes per well configured in current-clamp mode. The apical and basolateral bath solutions both contain (in mM) 140 NaCl, 5 KCl, 2 CaCl₂), 1 MgCl₂, 10 Hepes, and 10 glucose (adjusted to pH 7.4 with NaOH). To inhibit basal Na+ absorption, the ENaC inhibitor benzamil (10 μM) is added to the bath. Then, the adenylate cyclase activator, forskolin (10 μM), is added to the bath to activate CFTR. The forskolin-stimulated Cl− transport is halted by addition of CFTR inhibitor-172 (20 μM) to the bath at the end of the experiment to confirm specificity. VT and TEER recordings are digitally acquired at routine intervals using TECC or MTECC software (EP Design). VT and TEER are transformed into equivalent transepithelial Cl− current (IEQ), and the Area Under the Curve (AUC) of the IEQ time course between forskolin and CFTR inhibitor-172 addition is generated using Excel (Microsoft). Efficacy is expressed as the ratio of the test agent AUC divided by vehicle AUC. EC50s based on AUC are generated using the non-linear regression log(agonist) vs. response function in Prism software (Graphpad) with HillSlope fixed=1.

If a test agent increases the AUC of the forskolin-stimulated IEQ relative to VX-809 in CF-HBE cells, and this increase is inhibited by CFTR inhibitor-172, then the test agent is considered a CFTR corrector. TECC assay efficacy is expressed as the ratio of the test agent AUC divided by VX-809 AUC. As set forth in Table 2 below, a TECC value less than 3 is marked “A”; a value between 3 and 6 is marked “B”; a value between 6 and 20 is marked “C”.

TABLE 2

| Compound No | TECC | DSLS |
|---|---|---|
| 1 | | C |
| 2 | | C |
| 3 | | C |
| 4 | | A |
| 5 | | C |
| 6 | | B |
| 7 | A | C |
| 8A | | |
| 8B | C | C |
| 9 | | C |
| 10 | C | C |
| 11 | | C |
| 12 | | C |
| 13 | | B |
| 14 | B | C |
| 15 | A | C |
| 16 | | C |
| 17 | | B |
| 18 | | C |
| 19A | | C |
| 19B | C | C |
| 20A | C | A |
| 20B | C | B |
| 21 | B | C |
| 22 | B | C |
| 23 | | C |
| 24 | B | C |
| 24-2 | C | C |
| 26 | B | C |
| 27A | C | C |
| 27B | C | C |
| 27C | C | C |
| 28 | C | C |
| 29A | C | C |
| 29B | C | C |
| 30A | | C |
| 30B | | C |
| 31 | C | C |
| 32 | B | C |
| 33 | A | C |
| 34 | C | C |
| 35 | B | C |
| 36 | B | B |
| 37 | B | C |
| 38 | C | C |
| 39A | C | B |
| 39B | B | A |
| 40 | C | C |
| 41 | C | C |
| 42 | B | C |
| 43 | B | C |
| 44 | C | C |
| 45A | C | C |
| 45B | B | C |
| 46 | B | C |
| 47 | B | C |
| 48 | C | C |
| 49 | C | C |
| 50 | C | C |
| 51 | B | C |
| 52 | C | C |
| 53 | C | C |
| 54 | C | C |
| 55 | C | C |
| 56A | C | B |
| 56B | C | C |
| 57 | C | C |
| 58A | B | C |
| 58B | A | A |
| 59 | C | B |
| 60 | B | C |
| 61 | C | B |
| 62A | | A |
| 62B | A | C |
| 63 | A | C |
| 64 | A | C |
| 65 | C | C |
| 66 | B | C |
| 67A | A | C |
| 67B | A | A |
| 68 | A | B |
| 69 | A | C |
| 70 | A | A |
| 71 | C | C |
| 72 | B | C |
| 73A | B | C |
| 73B | A | A |
| 74 | C | C |
| 75 | A | B |
| 76 | A | A |
| 77 | | C |
| 78A | | A |
| 78B | | C |
| 79 | | A |
| 80 | C | C |
| 81 | | C |
| 82 | A | C |
| 83A | A | B |
| 83B | A | B |
| 84 | A | A |
| 85 | A | A |
| 86 | B | B |
| 87 | B | B |
| 88A | A | A |
| 88B | A | A |
| 89A | A | B |
| 89B | C | C |
| 89C | A | C |
| 89D | A | C |
| 90A | A | B |
| 90B | A | B |
| 91 | A | C |
| 92A | A | B |
| 92B | B | C |
| 93A | A | A |
| 93B | C | C |
| 94A | C | C |
| 94B | A | A |
| 95 | C | B |
| 96A | C | C |
| 96B | C | A |
| 97A | | |
| 97B | C | C |
| 98A | A | B |
| 98B | A | C |
| 99A | A | A |
| 99B | A | A |
| 100A | C | C |
| 100B | C | C |
| 101A | C | C |
| 101B | B | C |
| 102 | B | B |
| 103A | A | A |
| 103B | C | C |
| 104A | B | C |
| 104B | B | A |
| 105A | C | C |
| 105B | A | A |
| 106 | C | C |
| 107 | C | C |
| 108 | A | A |
| 109A | B | C |
| 109B | C | C |
| 110 | A | B |
| 111A | C | C |
| 111B | C | C |
| 112A | A | B |
| 112B | A | B |
| 113A | C | C |
| 113B | C | A |
| 114A | A | C |
| 114B | A | A |
| 115A | B | C |
| 115B | B | B |
| 116A | C | C |
| 116B | A | A |
| 117A | A | A |
| 117B | B | C |
| 118A | B | C |
| 118B | C | B |
| 119 | C | C |
| 120A | | C |
| 120B | | A |
| 121A | A | A |
| 121B | A | A |
| 121C | B | C |
| 122 | A | B |
| 123 | A | A |
| 124A | A | A |
| 124B | A | B |
| 125A | A | A |
| 125B | A | C |
| 125C | A | A |
| 125D | A | A |
| 126A | C | A |
| 126B | C | C |
| 127A | A | B |
| 127B | A | A |
| 127C | A | A |
| 127D | C | C |
| 128 | A | A |
| 129A | A | B |
| 129B | A | B |
| 130A | C | C |
| 130B | A | B |
| 131A | C | C |
| 131B | A | A |
| 132A | C | C |
| 132B | A | A |
| 133 | C | A |
| 134A | C | C |
| 134B | C | B |
| 135A | C | B |
| 135B | A | B |
| 136 | C | C |
| 137 | C | C |
| 135A | C | B |
| 135B | A | B |
| 136 | C | C |
| 137 | C | C |
| 138 | C | C |
| 139 | C | C |
| 140 | C | C |
| 141 | C | C |
| 142 | B | C |
| 143 | C | C |
| 144 | C | C |
| 145 | C | C |
| 146 | C | C |
| 147 | C | C |
| 148 | B | B |
| 149 | C | C |
| 150 | C | C |
| 151 | C | C |
| 152 | A | C |

EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

We claim:

1. A compound of Formula I: 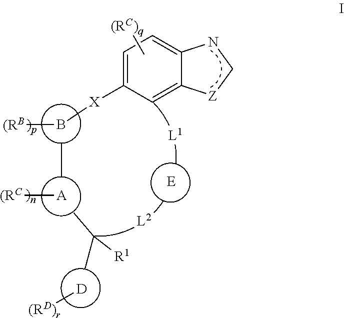
or a pharmaceutically acceptable salt thereof,
wherein
L¹ is an optionally substituted C₁₋₆ alkylene chain wherein 1-3 of the methylene units is optionally and independently replaced by —O—, —N(R²)—, —C(O)—, —S—, —S(O)—, an optionally substituted 3-6 membered carbocyclyl, 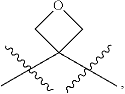 [SMILES: *C1(*)COC1]
optionally substituted C₂ alkenylene, or optionally substituted 5-6-membered heteroaryl;
L² is an optionally substituted C₁₋₆ alkylene chain wherein 1-3 of the methylene units is optionally and independently replaced by —C(CD₃)₂-, —O—, —N(R²)—, —C(O)—, —S—, —S(O)—, an optionally substituted 3-6 membered carbocyclyl, optionally substituted C₂ alkenylene, or optionally substituted 5-6-membered heteroaryl;
Ring A is a optionally substituted 5-membered heteroaryl comprising 1-4 heteroatoms selected from the group consisting of N, O or S;
Ring B is optionally substituted phenyl or optionally substituted 6-membered heteroaryl;
Ring D is optionally substituted phenyl or optionally substituted 5-6-membered heteroaryl;
Ring E is a optionally substituted 5-6-membered heteroaryl comprising 1-4 heteroatoms selected from the group consisting of N, O or S;
X is selected from the group consisting of —O—, —S—, —CH₂—, —C(OH)H—, —SO—, —CO—, —SO₂—, —CFH—, —CF₂—, and —N(R²)—;
each R^{A} is independently selected from the group consisting of halogen, cyano, optionally substituted C₁-C₆ aliphatic, optionally substituted C₁-C₆ alkoxy, and —CD₃;
each R^{B} is independently selected from the group consisting of halogen, cyano, —C(O)N(R²)₂, C(O)OR², —OR², —N(R²)₂, optionally substituted C₁-C₆ aliphatic and optionally substituted C₁-C₆ alkoxy;
each R^{C} is independently selected from the group consisting of hydrogen, halogen, cyano, optionally substituted C₁-C₆ aliphatic or optionally substituted C₁-C₆ alkoxy;
each R^{D} is independently selected from the group consisting of halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S, wherein each R^{D} is optionally substituted with 1-6 instances of R^{d};
wherein two instances of R^{D} may be taken together to form an optionally substituted 5-7 membered carbocyclic ring, optionally substituted 5-6-membered heteroaryl, and optionally substituted 3-6-membered heterocyclyl comprising 1-3 heteroatoms selected from the group consisting of N, O or S;
each R^{d} is independently selected from the group consisting of hydrogen, —OH, —CD₃, —C(O)N(R²)₂, C(O)OR², —OR², —N(R²)₂, —S(O)₂R² optionally substituted C₁-C₆ aliphatic, optionally substituted 5-6-membered heteroaryl, and optionally substituted 3-6-membered heterocyclyl comprising 1-3 heteroatoms selected from the group consisting of N, O or S;
R¹ is selected from the group consisting of hydrogen, cyano, —OR², —(CH₂)₀₋₃N(R²)₂, optionally substituted C₁-C₃ aliphatic, 3-6-membered heterocyclyl comprising 1-3 heteroatoms selected from the group consisting of N, O or S, and —CD₃;
each R² is independently selected from hydrogen, optionally substituted C₁-C₆ aliphatic, —OH, C₁-C₆ alkoxy, —S(O)₂(optionally substituted C₁-C₆ aliphatic);
Z is —CH═, —N═ or —NH—;
n is 0, 1,2 or 3;
p is 0, 1, 2, 3 or 4;
q is 1 or 2; and
r is 0, 1, 2, 3, 4 or 5.

2. The compound of claim 1, wherein Ring D is optionally substituted phenyl or optionally substituted pyridine.

3. The compound of claim 2, wherein Ring D is

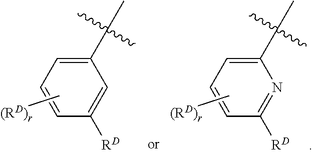

4. The compound of any of the previous claims, wherein Ring D is

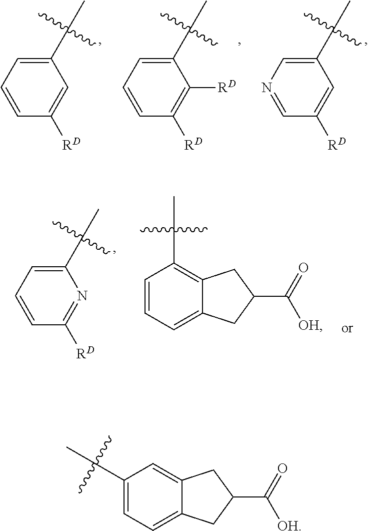

5. The compound of any of the previous claims, wherein Ring D is

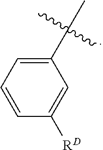

6. The compound of any of the previous claims, wherein Ring B is optionally substituted phenyl, optionally substituted pyridine, or optionally substituted pyridone.

7. The compound of any of the previous claims, wherein Ring B is 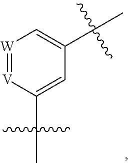
wherein
W is —CH═, —C(R^{B})═ or —N═; and
V is —CH═, —C(R^{B})═ or —N═.

8. The compound of any of the previous claims, wherein Ring B is

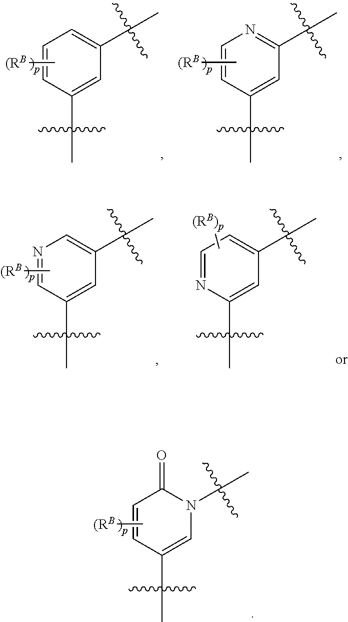

9. The compound of any of the previous claims, wherein Ring B is

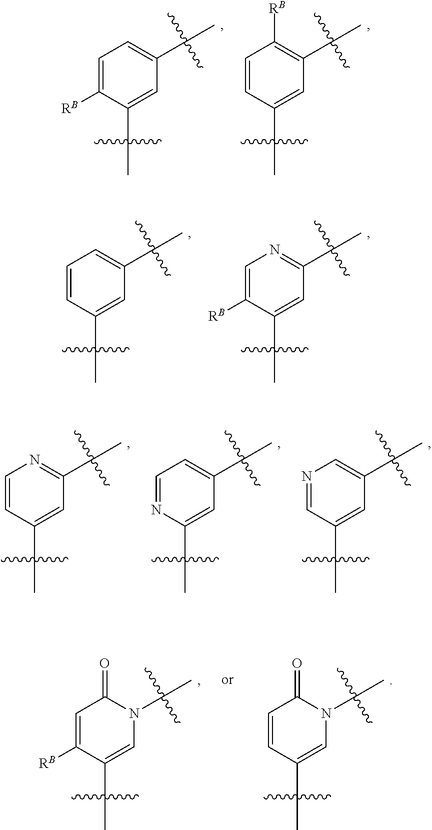

10. The compound of any of the previous claims, wherein Ring B is

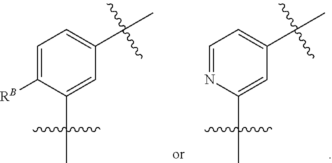

11. The compound of any of the previous claims, wherein R^{B} is halogen or optionally substituted C₁-C₃ alkyl.

12. The compound of any of the previous claims, wherein R^{B} is halogen.

13. The compound of any of the previous claims, wherein R^{B} is fluoro.

14. The compound of any of the previous claims, wherein the compound is of formula (I-a), (I-b), or (I-d): 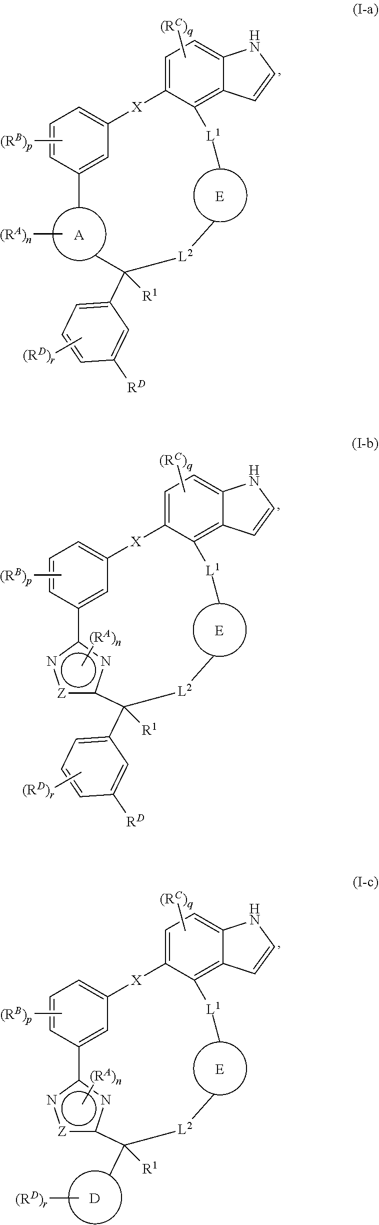
or a pharmaceutically acceptable salt thereof
wherein
Z is C or N.

15. The compound of any of the previous claims wherein the compound is of formula (I-a1), (I-a2), (I-a3), (I-a4), or (I-a5): 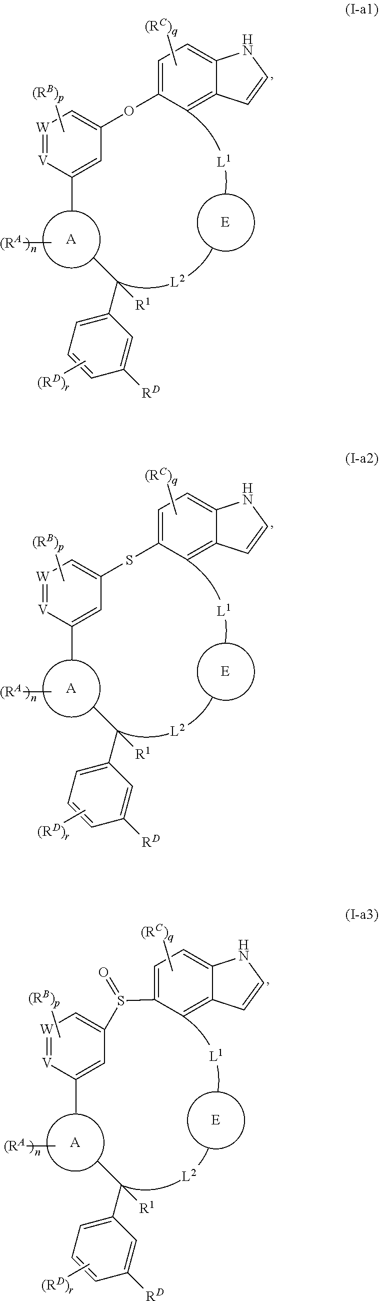 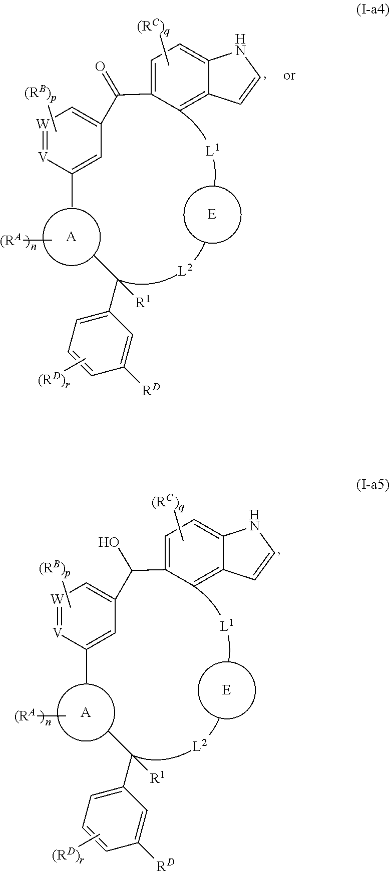
or a pharmaceutically acceptable salt thereof.

16. The compound of any of the previous claims wherein the compound is of formula (I-d1), (I-d2), (I-d3), (I-d4), or (I-d5) 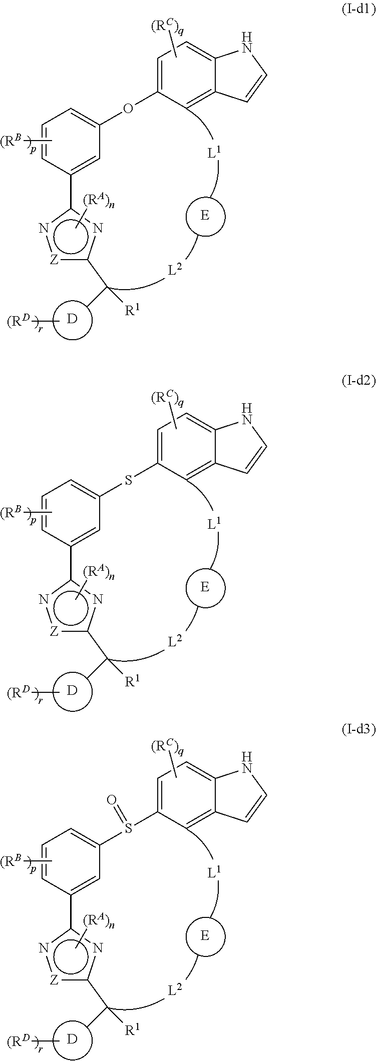 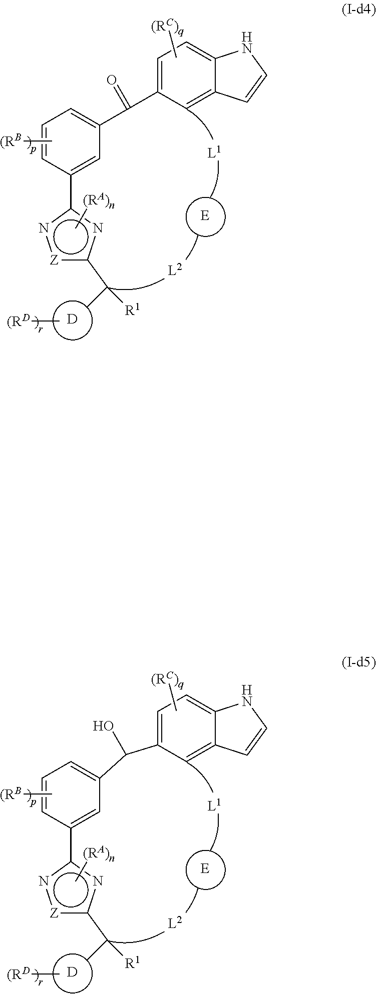
or a pharmaceutically acceptable salt thereof.

17. The compound of any of the previous claims, wherein Ring C is selected from optionally substituted indole, optionally substituted indazole, optionally substituted benzimidazole, optionally substituted 6-azaindole, and optionally substituted 7-azaindole.

18. The compound of any of the previously claims, wherein Ring C is optionally substituted indole.

19. The compound of any the previous claims, wherein at least one instance of R^{D} is 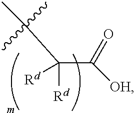
wherein
each R^{d} is independently hydrogen, optionally substituted methyl, —OH, —OMe, or —CD₃;
wherein, two instances R^{d} may, with the atoms on which they are attached, form a cyclopropyl ring; and
m is 0, 1, 2, or 3.

20. The compound of claim 16, wherein each R^{d} is independently hydrogen, methyl, —CF₃, —CF₂H, or —CFH₂.

21. The compound of claim 17, wherein each R^{d} is independently selected from hydrogen and methyl.

22. The compound of any of the previous claims, wherein at least one instance of R^{D} is selected from the group consisting of

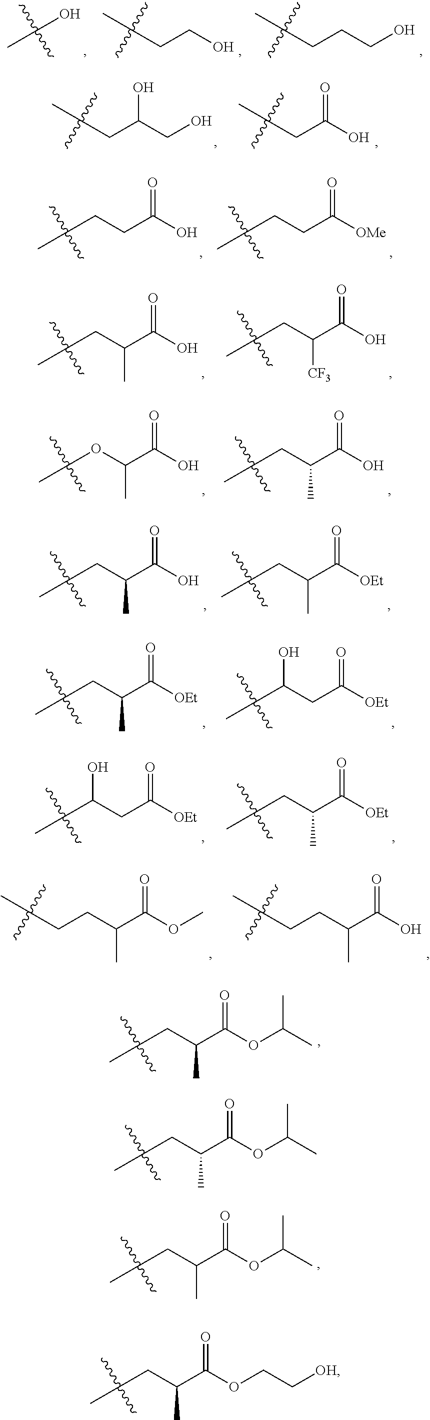

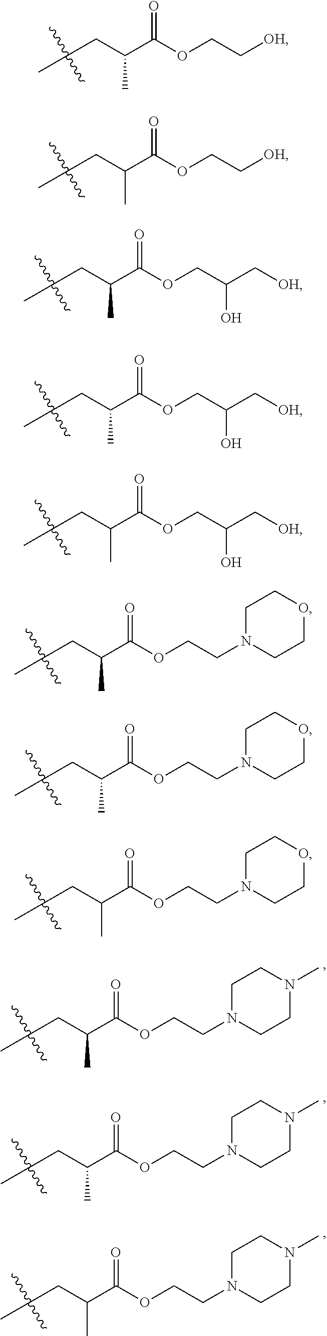

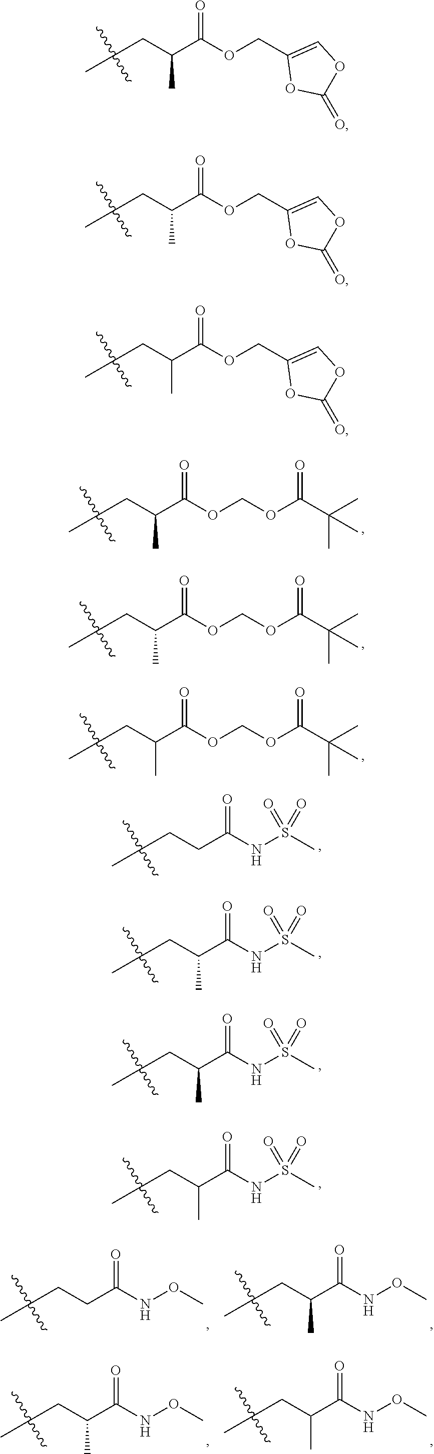

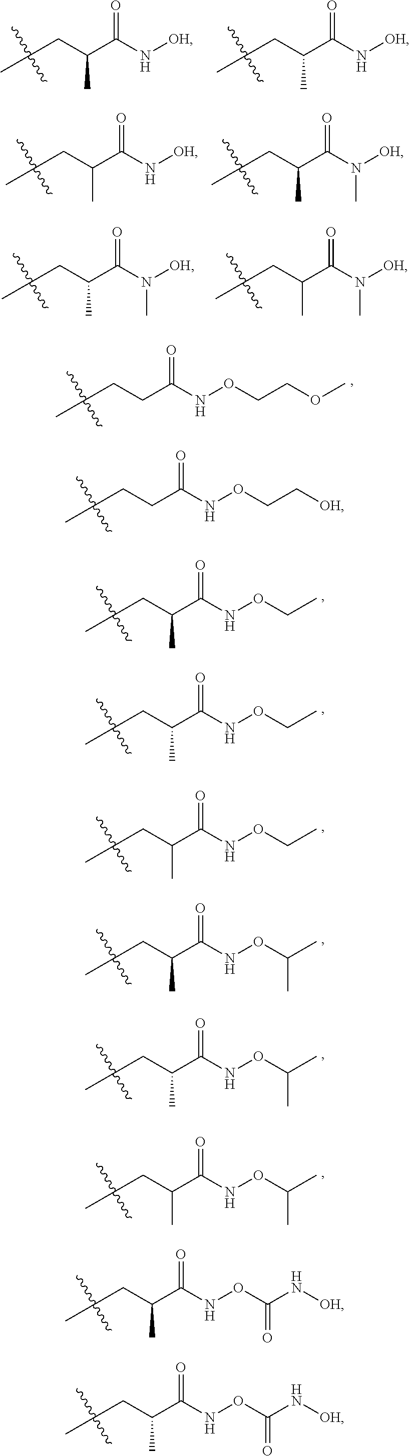

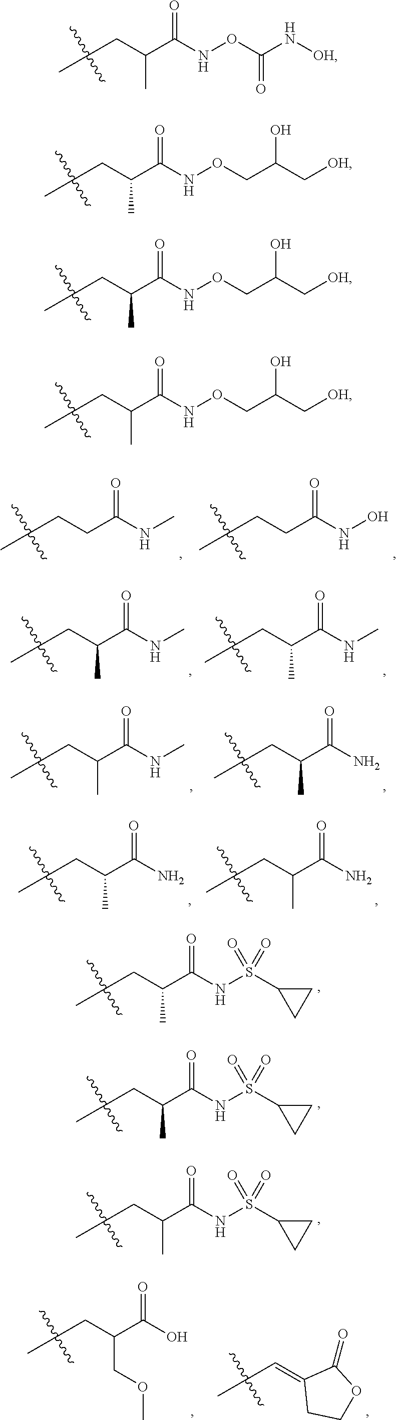

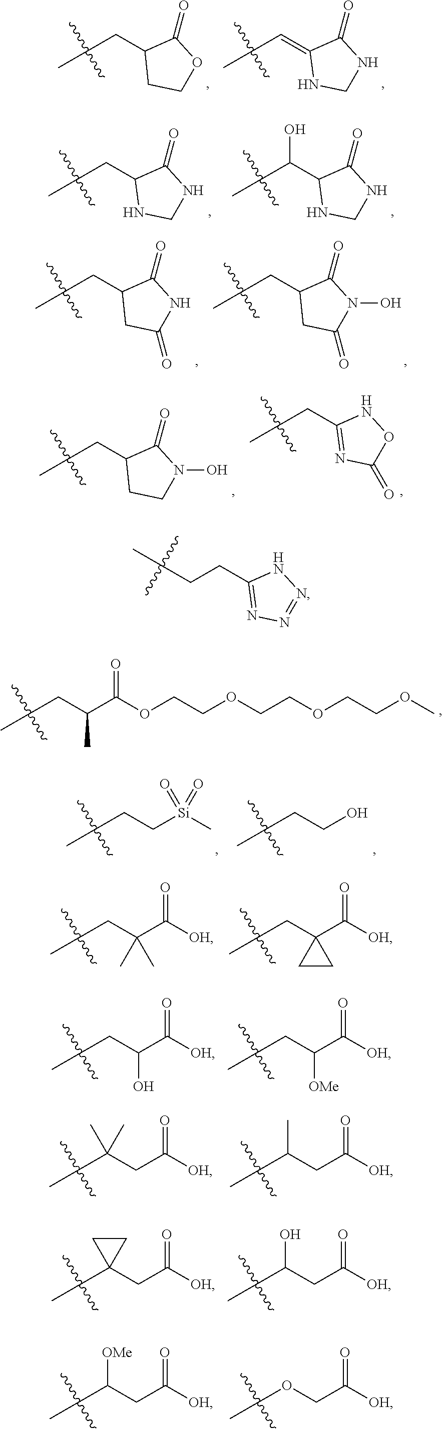

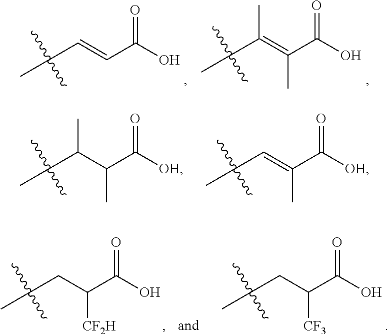

23. The compound of claim 19, wherein at least one instance of R^{D} is selected from the group consisting of

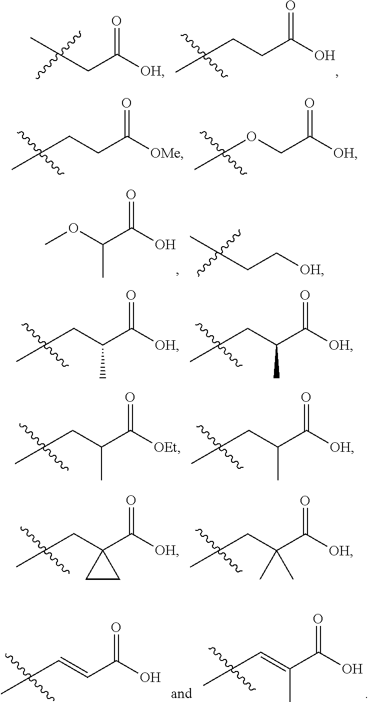

24. The compound of claim 20, wherein R^{D} is

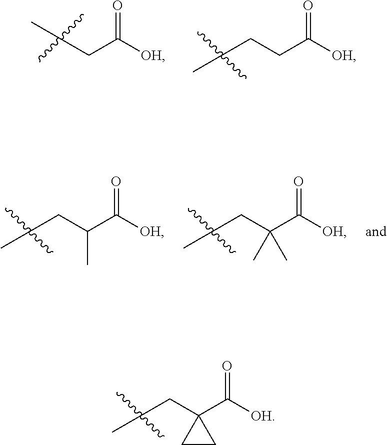

25. The compound of claim 21, wherein R^{D} is

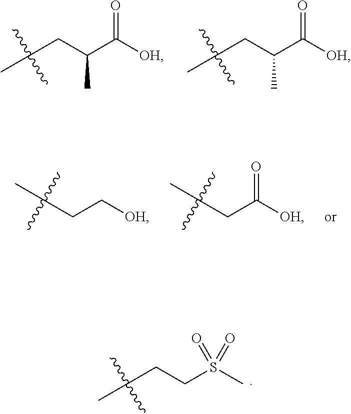

26. The compound of claim 21, wherein R^{D} is

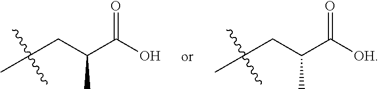

27. The compound of any of the previous claims wherein, ring A is selected from the group consisting of imidazole, pyrazole, tetrazole, oxazole, thiazole, and 1, 2, 4 triazole.

28. The compound of any of the previous claims, wherein each R^{A} is independently selected from hydrogen, methyl and —CD₃.

29. The compound of any of the previous claims, wherein the compound is of formula (I-e): 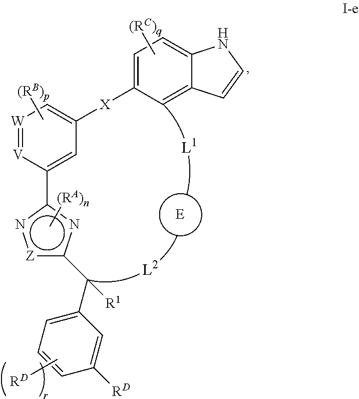
or a pharmaceutically acceptable salt thereof.

30. The compound of any of the previous claims, wherein L¹ is an optionally substituted C₁₋₆ alkylene chain and L² is an optionally substituted C₁₋₆ alkylene chain, wherein one of the methylene units of L² is optionally replaced with —O—.

31. The compound of any of the previous claims, wherein L¹ is a C₁₋₆ alkylene chain substituted with 1-3 instances of methyl, and L² is C₁₋₆ alkylene chain, wherein one of the methylene units of L² is optionally replaced with —O— and wherein L² is optionally substituted with 1-3 instances of methyl.

32. The compound of any of the previous claims, wherein L¹ is an unsubstituted C₂ alkylene chain.

33. The compound of any of the previous claims, wherein L² is an C₅ alkylene chain, wherein one of the methylene units of L² is optionally replaced with —O— and wherein L² is optionally substituted with 1-3 instances of methyl.

34. The compound of any of the previous claims, wherein L² is a C₅ alkylene chain, wherein L² is optionally substituted with 1-3 instances of methyl.

35. The compound of any of the previous claims, wherein L² is a C₅ alkylene chain, wherein L² is optionally substituted with 1-3 instances of methyl.

36. The compound of any of the previous claims, wherein the compound is of formula (I-f) or (I-f′): 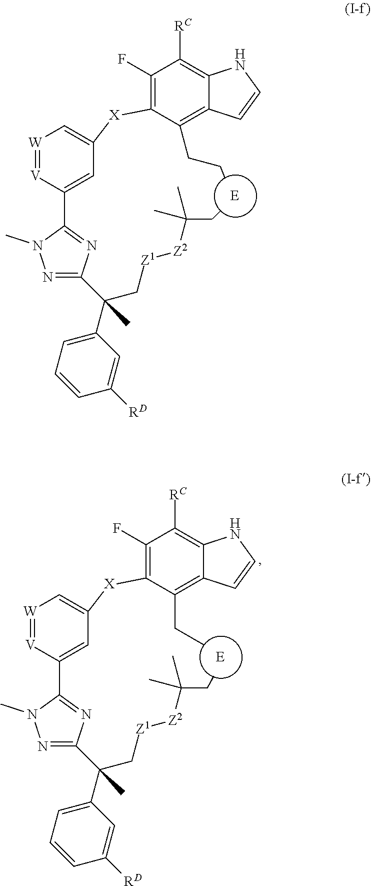
or a pharmaceutically acceptable salt thereof
wherein Z¹ is —CH₂— or —O—; and
Z² is —CH₂— or —O—.

37. The compound of any of the previous claims, wherein the compound is of formula (I-g) or (I-h): 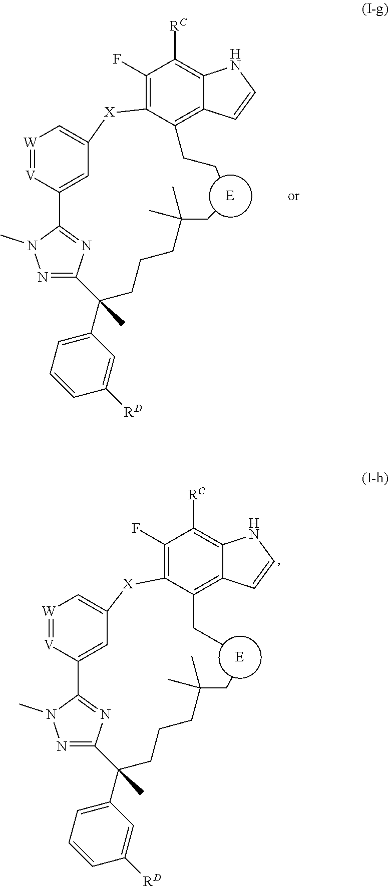
or a pharmaceutically acceptable salt thereof.

38. The compound of any of the previous claims, wherein the compound is of formula (I-g1), (I-g2), (I-h1), or (I-h2) 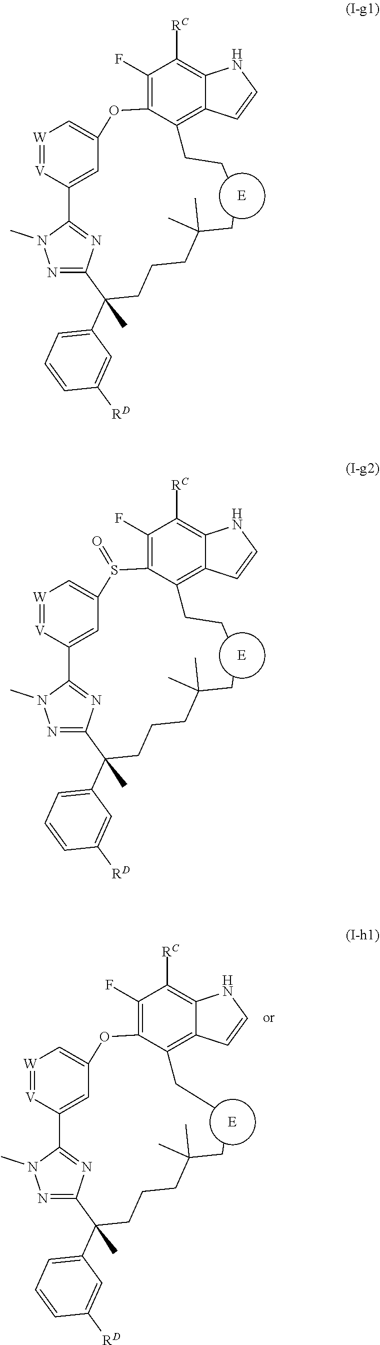 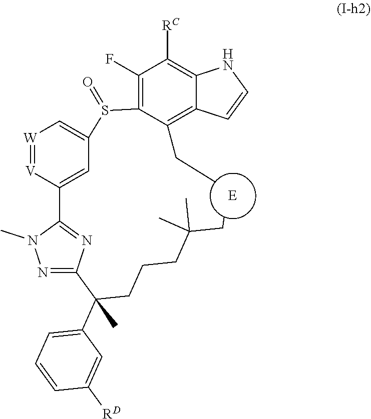
or a pharmaceutically acceptable salt thereof.

39. The compound of any of the previous claims, wherein the compound is of formula (I-i) or (I-j): 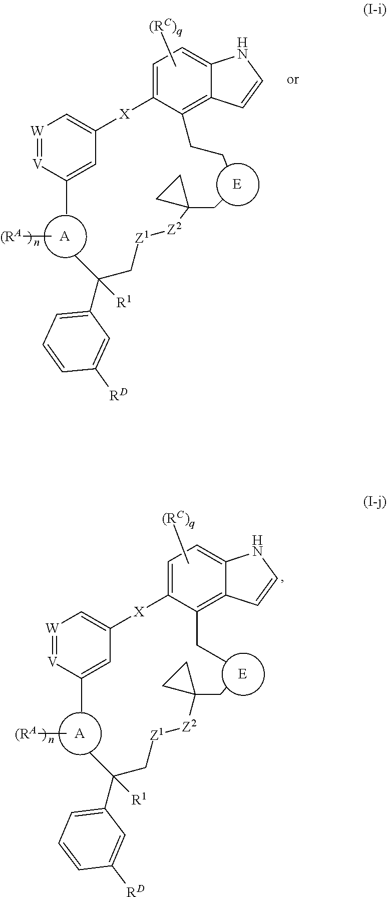
or a pharmaceutically acceptable salt thereof.

40. The compound of any of the previous claims, wherein the compound is of formula (I-k) or (I-l): 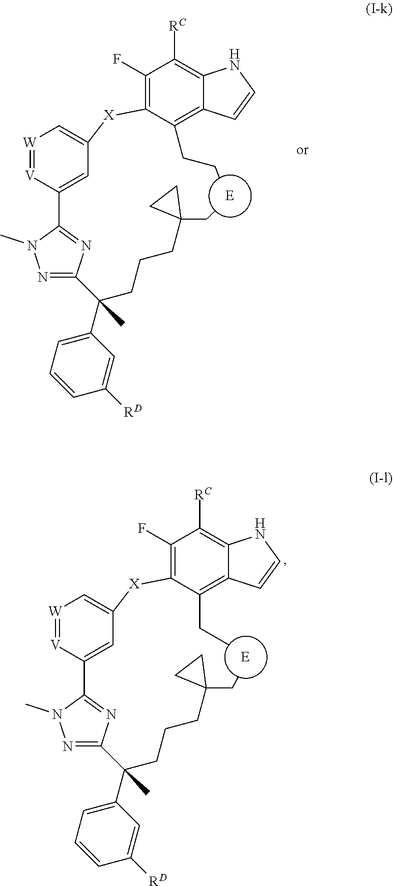
or a pharmaceutically acceptable salt thereof.

41. The compound of any of the previous claims, wherein Z¹ is —O— and Z² is —CH₂—.

42. The compound of any of the previous claims, wherein Z¹ is —CH₂— and Z² is —O—.

43. The compound of any of the previous claims, wherein Z¹ is —CH₂— and Z² is —CH₂—.

44. The compound of any of the previous claims, wherein R¹ is optionally substituted C₁-C₃ aliphatic.

45. The compound of any of the previous claims, wherein R¹ is optionally substituted methyl or —CD₃.

46. The compound of any of the previous claims, wherein each R^{d} is independently hydrogen or optionally substituted C₁-C₃ aliphatic.

47. The compound of any of the previous claims, wherein at least one R^{d} is optionally substituted C₁-C₃ aliphatic.

48. The compound of any of the previous claims, wherein at least one R^{d} is methyl.

49. The compound of any of the previous claims, wherein each R^{A} is independently hydrogen or optionally substituted C₁-C₃ aliphatic.

50. The compound of any of the previous claims, wherein each R^{A} is independently hydrogen, methyl or —CD₃.

51. The compound of any of the previous claims, wherein R^{C} is hydrogen or halogen.

52. The compound of any of the previous claims, wherein R^{C} is hydrogen or fluoro.

53. The compound of any of the previous claims, wherein R^{C} is hydrogen.

54. The compound of any of the previous claims, wherein R^{D} is halogen.

55. The compound of any of the previous claims, wherein R^{D} is fluoro.

56. The compound of any of the previous claims, wherein R^{C} is fluoro.

57. The compound of any of the previous claims, wherein Ring E is selected from the group consisting of furan, pyrrole, thiophene, pyrazole, oxazole, thiazole, imidazole, triazole, tetrazole, oxadiazole, pyridine, pyrazine and thiadiazole.

58. The compound of any of the previous claims, wherein Ring E is selected from the group consisting of oxazole, pyrazole, and triazole.

59. A compound selected from a compound of Table 1 or a pharmaceutically acceptable salt thereof.

60. A pharmaceutical composition comprising a compound of any of the previous claims and a pharmaceutically acceptable excipient.

61. A method of treating a CFTR-mediated disease or disorder comprising administering a patient in need there of a compound any of claims 1-60 or a pharmaceutical composition of claim 60.

62. The method of claim 61, wherein the disease or condition is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear plasy, Pick's disease, several polyglutamine neurological disorders, Huntington's, spinocerebullar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

63. The method of claim 61 or 62, wherein the disease or condition is selected from cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

64. The method of any one of claims 61-63, wherein the disease or condition is cystic fibrosis.

65. A method of treating cystic fibrosis in a subject, comprising administering to the subject a therapeutically effective amount of a compound of any one of claims 1-59, or the pharmaceutical composition of claim 60.

66. The method of claim 65, wherein the subject is human.
